(19) United States
(12) Patent Application Publication
(10) Pub. No.: US 2023/0077362 A1
(43) Pub. Date: Mar. 16, 2023

(54) MOLECULES HAVING CERTAIN PESTICIDAL UTILITIES, AND INTERMEDIATES, COMPOSITIONS, AND PROCESSES RELATED THERETO

(71) Applicant: CORTEVA AGRISCIENCE LLC, INDIANAPOLIS, IN (US)

(72) Inventors: NATALIE C. GIAMPIETRO, CARMEL, IN (US); DAVID A. DEMETER, FISHERS, IN (US); ABDALLAH BACHIR DIAGNE, INDIANAPOLIS, IN (US); KENNETH VIRGEL N. ESGUERRA, ZIONSVILLE, IN (US); RONALD J. HEEMSTRA, FISHERS, IN (US); RYAN AARON SCHULDT, AVON, IN (US); THOMAS J. BARTON, INDIANAPOLIS, IN (US); LINDSEY G. HORTY, INDIANAPOLIS, IN (US); THOMAS C. SPARKS, GREENFIELD, IN (US); GERALD B. WATSON, ZIONSVILLE, IN (US)

(73) Assignee: CORTEVA AGRISCIENCE LLC, INDIANAPOLIS, IN (US)

(21) Appl. No.: 17/626,865

(22) PCT Filed: Jul. 16, 2020
(86) PCT No.: PCT/US20/42237

§ 371 Date: Jan. 13, 2022

Related U.S. Application Data
(60) Provisional application No. 62/875,079, filed on Jul. 17, 2019.

(51) Int. Cl. A01N 43/78; C07D 417/12; C07D 419/12; A01N 43/82; C07D 419/14; A01P 7/02; A01P 7/04

(52) U.S. Cl. CPC A01N 43/78; C07D 417/12; C07D 419/12; A01N 43/82; C07D 419/14; A01P 7/02; A01P 7/04

(57) ABSTRACT

This disclosure relates to compounds having pesticidal utility against pests in phyla Nematoda, Arthropoda, and/or Mollusca, processes to produce such compounds and intermediates used in such processes, compositions containing such compounds, and processes of using such compounds against such pests. These compounds/molecules may be used, for example, as nematicides, acaricides, insecticides, miticides, and/or molluscicides. This document discloses compounds having the following formula (“Formula One” and/or “Formula One-A”).

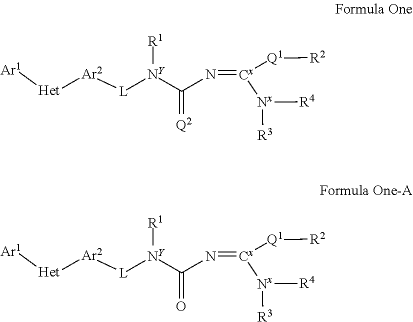

### CROSS REFERENCES TO RELATED APPLICATIONS

This application claims the benefit of U.S. Provisional Patent Application Ser. No. 62/875,079, filed Jul. 17, 2019.

### FIELD OF THE DISCLOSURE

This disclosure relates to the field of molecules having pesticidal utility against pests in Phyla Nematoda, Arthropoda, and Mollusca, processes to produce such molecules and intermediates used in such processes, compositions containing such molecules, and processes of using such molecules against such pests. These molecules may be used, for example, as nematicides, acaricides, insecticides, miticides, and molluscicides.

### BACKGROUND OF THE DISCLOSURE

“Many of the most dangerous human diseases are transmitted by insect vectors” (Rivero, A. et al., Insect Control of Vector-Borne Diseases: When is Insect Resistance a Problem? Public Library of Science Pathogens, 6(8) (2010)). Historically, vector-borne diseases, such as, malaria, dengue, yellow fever, plague, and louse-borne typhus, among others, were responsible for more human disease and death from the 1600's through the early 1900's than all other causes combined (Gubler D., Resurgent Vector-Borne Diseases as a Global Health Problem, Emerging Infectious Diseases, Vol. 4, No. 3, July-September (1998)). Currently, vector-borne diseases are responsible for about 17% of the global parasitic and infectious diseases. It has been estimated that about 250 million people around the world have malaria and about 800,000 deaths occur each year—85% of those deaths are children under the age of five. A further 250,000 to 500,000 cases of dengue hemorrhagic fever occur each year (Matthews, G., Integrated Vector Management: controlling vectors of malaria and other insect vector borne diseases (2011)). Vector control plays a critical role in the prevention and control of infectious diseases. However, insecticide resistance, including resistance to multiple insecticides, has arisen in all insect species that are major vectors of human diseases (Rivero, A. et al.).

Each year insects, plant pathogens, and weeds destroy more than 40% of all potential food production. This loss occurs despite the application of pesticides and the use of a wide array of non-chemical controls, such as crop rotations and biological controls. If just some of this food could be saved, it could be used to feed the more than three billion people in the world who are malnourished (Pimental, D., Pest Control in World Agriculture, Agricultural Sciences—Vol. II (2009)).

Plant parasitic nematodes are among the most widespread pests, and are frequently one of the most insidious and costly. It has been estimated that losses attributable to nematodes are from about 9% in developed countries to about 15% in undeveloped countries. However, in the United States of America, a survey of 35 States on various crops indicated nematode-derived losses of up to 25% (Nicol, J. et al., Current Nematode Threats to World Agriculture, Genomic and Molecular Genetics of Plant-Nematode Interactions (Eds. Jones, J. et al.), Chapter 2, (2011)).

It is noted that gastropods (slugs and snails) are pests of less economic importance than insects or nematodes, but in certain areas, gastropods may reduce yields substantially, severely affecting the quality of harvested products, as well as transmitting human, animal, and plant diseases. While only a few dozen species of gastropods are serious regional pests, a handful of species are important pests on a world-wide scale. In particular, gastropods affect a wide variety of agricultural and horticultural crops, such as arable, pastoral, and fiber crops; vegetables; bush and tree fruits; herbs; and ornamentals (Speiser, B., Molluscicides, Encyclopedia of Pest Management (2002)).

Termites cause damage to all kinds of private and public structures, as well as to agricultural and forestry resources. In 2003, it was estimated that termites cause over US$20 billion in damage world-wide each year (Su, N.Y., Overview of the global distribution and control of the Formosan subterranean termite, Sociobiology 2003, 41, 177-192).

Therefore, for many reasons, including the above reasons, a need exists for new pesticides.

### Definitions

The examples given in the definitions are generally non-exhaustive and must not be construed as limiting the molecules disclosed in this document. It is understood that a substituent should comply with chemical bonding rules and steric compatibility constraints in relation to the particular molecule to which it is attached.

“Alkenyl” means an acyclic, unsaturated (at least one carbon-carbon double bond), branched or unbranched, substituent consisting of carbon and hydrogen, for example, vinyl, allyl, butenyl, pentenyl, and hexenyl.

“Alkenyloxy” means an alkenyl further consisting of a carbon-oxygen single bond, for example, allyloxy, butenyloxy, pentenyloxy, hexenyloxy.

“Alkoxy” means an alkyl further consisting of a carbon-oxygen single bond, for example, methoxy, ethoxy, propoxy, isopropoxy, butoxy, isobutoxy, and tert-butoxy.

“Alkyl” means an acyclic, saturated, branched or unbranched, substituent consisting of carbon and hydrogen, for example, methyl, ethyl, propyl, isopropyl, butyl, and tert-butyl.

“Alkynyl” means an acyclic, unsaturated (at least one carbon-carbon triple bond), branched or unbranched, substituent consisting of carbon and hydrogen, for example, ethynyl, propargyl, butynyl, and pentynyl.

“Alkynyloxy” means an alkynyl further consisting of a carbon-oxygen single bond, for example, pentynyloxy, hexynyloxy, heptynyloxy, and octynyloxy.

“Aryl” means a cyclic, aromatic substituent consisting of hydrogen and carbon, for example, phenyl, naphthyl, and biphenyl.

“Cycloalkenyl” means a monocyclic or polycyclic, unsaturated (at least one carbon-carbon double bond) substituent consisting of carbon and hydrogen, for example, cyclobutenyl, cyclopentenyl, cyclohexenyl, norbornenyl, bicyclo[2.2.2]octenyl, tetrahydronaphthyl, hexahydronaphthyl, and octahydronaphthyl.

“Cycloalkenyloxy” means a cycloalkenyl further consisting of a carbon-oxygen single bond, for example, cyclobutenyloxy, cyclopentenyloxy, norbornenyloxy, and bicyclo[2.2.2]octenyloxy.

“Cycloalkyl” means a monocyclic or polycyclic, saturated substituent consisting of carbon and hydrogen, for example, cyclopropyl, cyclobutyl, cyclopentyl, norbornyl, bicyclo[2.2.2]octyl, and decahydronaphthyl.

“Cycloalkoxy” means a cycloalkyl further consisting of a carbon-oxygen single bond, for example, cyclopropyloxy, cyclobutyloxy, cyclopentyloxy, norbornyloxy, and bicyclo[2.2.2]octyloxy.

“Halo” means fluoro, chloro, bromo, and iodo.

“Haloalkoxy” means an alkoxy further consisting of, from one to the maximum possible number of identical or different, halos, for example, fluoromethoxy, trifluoromethoxy, 2,2-difluoropropoxy, chloromethoxy, trichloromethoxy, 1,1,2,2-tetrafluoroethoxy, and pentafluoroethoxy.

“Haloalkyl” means an alkyl further consisting of, from one to the maximum possible number of, identical or different, halos, for example, fluoromethyl, trifluoromethyl, 2,2-difluoropropyl, chloromethyl, trichloromethyl, and 1,1,2,2-tetrafluoroethyl.

“Heterocyclyl” means a cyclic substituent that may be fully saturated, partially unsaturated, or fully unsaturated, where the cyclic structure contains at least one carbon and at least one heteroatom, where said heteroatom is nitrogen, sulfur, or oxygen. Examples of aromatic heterocyclyls include, but are not limited to, benzofuranyl, benzoisothiazolyl, benzoisoxazolyl, benzoxazolyl, benzothienyl, benzothiazolyl, cinnolinyl, furanyl, indazolyl, indolyl, imidazolyl, isoindolyl, isoquinolinyl, isothiazolyl, isoxazolyl, oxadiazolyl, oxazolinyl, oxazolyl, phthalazinyl, pyrazinyl, pyrazolinyl, pyrazolyl, pyridazinyl, pyridyl, pyrimidinyl, pyrrolyl, quinazolinyl, quinolinyl, quinoxalinyl, tetrazolyl, thiazolinyl, thiazolyl, thienyl, triazinyl, and triazolyl. Examples of fully saturated heterocyclyls include, but are not limited to, piperazinyl, piperidinyl, morpholinyl, pyrrolidinyl, tetrahydrofuranyl, and tetrahydropyranyl. Examples of partially unsaturated heterocyclyls include, but are not limited to, 1,2,3,4-tetrahydro-quinolinyl, 4,5-dihydro-oxazolyl, 4,5-dihydro-1H-pyrazolyl, 4,5-dihydro-isoxazolyl, and 2,3-dihydro-[1,3,4]-oxadiazolyl.

### DETAILED DESCRIPTION OF THE DISCLOSURE

Provided are compounds/molecules having a structure of “Formula One” or “Formula One-A”:

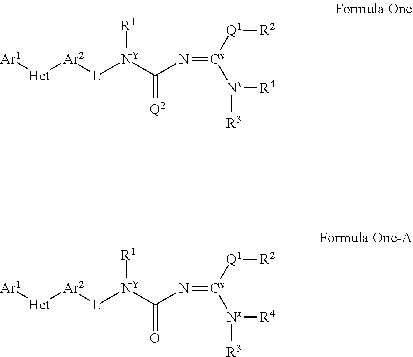

wherein:
(A) Ar¹ is selected from

(1) furanyl, phenyl, pyridazinyl, pyridyl, pyridinonyl, pyrimidinyl, thienyl, or

(2) substituted furanyl, substituted phenyl, substituted pyridazinyl, substituted pyridyl, substituted pyridinonyl, substituted pyrimidinyl, or substituted thienyl,
wherein said substituted furanyl, substituted phenyl, substituted pyridazinyl, substituted pyridyl, substituted pyridinonyl, substituted pyrimidinyl, and substituted thienyl have one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)OC₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
   (B) Het is a 5- or 6-membered, saturated or unsaturated, heterocyclic ring, containing one or more heteroatoms independently selected from nitrogen, sulfur, or oxygen, where said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O) C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, or S(═O)₂NR^{x}R^{y},

wherein each alkyl, haloalkyl, cycloalkyl, halocycloalkyl, alkoxy, haloalkoxy, alkenyl, cycloalkenyl, haloalkenyl, alkynyl, phenyl, and phenoxy substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)C(═O)NR^{x}R^{y}, (C₁-C₈ alkyl)NR^{x}R^{y}, C(═O)C(═O)(C₁-C₈ alkyl), C(═O)O(C₁-C₈ alkyl), C(═O)C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, or S(═O)₂NR^{x}R^{y};

(C) Ar² is selected from

(1) furanyl, phenyl, pyridazinyl, pyridyl, pyrimidinyl, thienyl, or

(2) substituted furanyl, substituted phenyl, substituted pyridazinyl, substituted pyridyl, substituted pyrimidinyl, or substituted thienyl,
wherein said substituted furanyl, substituted phenyl, substituted pyridazinyl, substituted pyridyl, substituted pyrimidinyl, and substituted thienyl, have one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
   (D) R¹ is selected from H, CN, OH, SH, NO₂, C(═O)H, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1),

wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);

(E) R² is selected from (J), H, C₁-C₈ alkyl, C₃-C₈ cycloalkyl, C₂-C₈ alkenyl, C₂-C₈ alkynyl, C(═O)(C₁-C₈ alkyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), C₁-C₈ alkyl)S(═O)₂(C₁-C₈ alkyl), (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈ alkyl)-C(═O)N(R^{x})(C₁-C₈ alkyl)-(Het-1), (C₁-C₈ alkyl)-C(═O)(Het-1), (C₁-C₈ alkyl)-C(═O)N(R^{x})(C₁-C₈ alkyl)N(R^{y})C(═O)OH, (C₁-C₈ alkyl)-C(═O)N(R^{x})(C₁-C₈ alkyl)N(R^{x})(R^{y}), (C₁-C₈ alkyl)-C(═O)N(R^{x})(C₁-C₈ alkyl)N(R^{y})C(═O)O—(C₁-C₈ alkyl), (C₁-C₈ alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(Het-1), (C₁-C₈ alkyl)S(═O)(Het-1), (C₁-C₈ alkyl)S(═O)₂(Het-1), or (C₁-C₈ alkyl)-O-(Het-1),

wherein each alkyl, cycloalkyl, phenyl, and (Het-1) are optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)OH, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);

(F) R³ is selected from substituted C₃-C₈ cycloalkyl, substituted phenyl, substituted (C₁-C₈ alkyl)phenyl, substituted (C₁-C₈ alkyl)-O-phenyl, substituted (C₂-C₈ alkenyl)-O-phenyl, substituted (Het-1), substituted (C₁-C₈ alkyl)-(Het-1), or substituted (C₁-C₈ alkyl)-O-(Het-1),

wherein said substituted C₃-C₈ cycloalkyl, substituted phenyl, substituted (C₁-C₈ alkyl)phenyl, substituted (C₁-C₈ alkyl)-O-phenyl, substituted (C₂-C₈ alkenyl)-O-phenyl, substituted (Het-1), substituted (C₁-C₈ alkyl)-(Het-1), or substituted (C₁-C₈ alkyl)-O-(Het-1) must have at least one substituent selected from NR^{x}C(═O)(C₁-C₈ alkyl), OH, SH, S(C₁-C₈ alkyl), S(C₃-C₈ cycloalkyl), S(C₁-C₈ haloalkyl), S(C₃-C₈ halocycloalkyl), C₁-C₈ haloalkyl, (C₁-C₈ alkyl)O(C₁-C₈ alkyl), (C₁-C₈ alkyl)O(C₁-C₈ alkyl)(C₃-C₈ cycloalkyl), unsubstituted (C₁-C₈ alkyl)O(C₁-C₈ alkyl)phenyl, (C₁-C₈ alkyl)O(C₁-C₈ alkyl)phenyl substituted with one of F, Cl, (C₁-C₈ haloalkyl), and (C₁-C₈ haloalkoxy), (C₁-C₈ alkyl)O(C₁-C₈ alkyl)benzothiazolyl, (C₁-C₈ alkyl)O(C₁-C₈ alkyl)benzoxazolyl, (C₁-C₈ alkyl)O(C₁-C₈ alkyl)thiophenyl, (C₁-C₈ alkyl)O(C₁-C₈ alkyl)thienopyrazolyl, (C₁-C₈ alkyl)O(C₁-C₈ haloalkyl), (C₁-C₈ haloalkyl)O(C₁-C₈ alkyl), (C₁-C₈ haloalkyl)O(C₁-C₈ haloalkyl), (C₁-C₈ alkyl)S(C₁-C₈ alkyl), (C₁-C₈ alkyl)S(C₁-C₈ haloalkyl), (C₁-C₈ alkyl)S(═O)(C₁-C₈ alkyl), (C₁-C₈ alkyl)S(═O)(C₁-C₈ haloalkyl), (C₁-C₈ alkyl)S(═O)₂(C₁-C₈ alkyl), (C₁-C₈ alkyl)S(═O)₂(C₁-C₈ haloalkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), unsubstituted (C₁-C₈ alkyl)phenyl, (C₁-C₈ alkyl)phenyl substituted with one or more of F and (C₁-C₈)haloalkyl, (C₁-C₈ alkyl)pyridyl, or (C₁-C₈ alkyl)-O-phenyl; and

said substituted C₃-C₈ cycloalkyl, substituted phenyl, substituted (C₁-C₈ alkyl)phenyl, substituted (C₁-C₈ alkyl)-O-phenyl, substituted (C₂-C₈ alkenyl)-O-phenyl, substituted (Het-1), substituted (C₁-C₈ alkyl)-(Het-1), or substituted (C₁-C₈ alkyl)-O-(Het-1) may be optionally substituted with one or more additional substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ haloalkyl)O(C₁-C₈ alkyl), (C₁-C₈ haloalkyl)O(C₁-C₈ haloalkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);

(G) R⁴ is selected from (J), H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈ alkyl)-C(═O)N(R^{x})(C₁-C₈ alkyl)-(Het-1), (C₁-C₈ alkyl)-C(═O)(Het-1), (C₁-C₈ alkyl)-C(═O)N(R^{x})(C₁-C₈ alkyl)N(R^{y})C(═O)OH, (C₁-C₈ alkyl)-C(═O)N(R^{x})(C₁-C₈ alkyl)N(R^{x})(R^{y}), (C₁-C₈ alkyl)-C(═O)N(R^{x})(C₁-C₈ alkyl)N(R^{y})C(═O)O—(C₁-C₈ alkyl), (C₁-C₈ alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(═O)(Het-1), (C₁-C₈ alkyl)S(═O)₂(Het-1), or (C₁-C₈ alkyl)-O-(Het-1),

wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1);

(H) each of Q¹ and Q² is independently selected from O or S;
(I) R^{x} and R^{y} are independently selected from H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-C(═O)(Het-1), (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-S-(Het-1), (C₁-C₈ alkyl)S(═O)(Het-1), (C₁-C₈ alkyl)S(═O)₂(Het-1), or (C₁-C₈ alkyl)-O-(Het-1),

wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1),

or R^{x} and R^{y} together can optionally form a 5- to 7-membered saturated or unsaturated cyclic group which may contain one or more heteroatoms selected from nitrogen, sulfur, and oxygen, and where said cyclic group may be substituted with H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, and (Het-1);

(J) R² and R⁴ may be a 1- to 4-membered saturated or unsaturated, hydrocarbyl link, which may contain one or more heteroatoms selected from nitrogen, sulfur, and oxygen, and together with C^{x}(Q′)(N^{x}) forms a 4- to 7-membered cyclic structure, wherein said hydrocarbyl link may optionally be substituted with one or more substituents independently selected from R⁵, R⁶, and R⁷, wherein each R⁵, R⁶, and R⁷ is selected from H, F, Cl, Br, I, CN, OH, SH, NO₂, oxo, thioxo, NR^{x}R^{y}, C₁-C₈ alkyl, C₁-C₈ alkyl substituted with at least one OH, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, or (Het-1);
(K) (Het-1) is a 5- or 6-membered, saturated or unsaturated, heterocyclic ring, containing one or more heteroatoms independently selected from nitrogen, sulfur or oxygen, wherein said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy,

wherein each alkyl, cycloalkyl, alkoxy, alkenyl, alkynyl, phenyl, and phenoxy may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), OSO₂(C₁-C₈ alkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy; and

(L) L is a linker selected from

(1) a bond,

(2) a saturated or unsaturated, substituted or unsubstituted, linear (C₁-C₄)hydrocarbyl linker, or

(3) a saturated or unsaturated, substituted or unsubstituted, cyclic (C₃-C₈)hydrocarbyl group linker,

wherein each of said linkers connects Ar² to N^{Y} and

wherein said substituted linear (C₁-C₄)hydrocarbyl linker and substituted cyclic (C₃-C₈)hydrocarbyl linker has one or more substituents independently selected from R⁸, R⁹, R¹⁰, R¹¹, and R¹², wherein each R⁸, R⁹, R¹⁰, R¹¹, and R¹², is selected from H, F, Cl, Br, I, CN, OH, SH, NO₂, oxo, thioxo, NR^{x}R^{y}, C₁-C₈ alkyl, C₁-C₈ haloalkyl, C₁-C₈ alkoxy, C₁-C₈ haloalkoxy, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₃-C₈ cycloalkenyl, C₃-C₈ halocycloalkenyl, S(C₁-C₈ alkyl), S(C₃-C₈ cycloalkyl), S(C₁-C₈ haloalkyl), S(C₃-C₈ halocycloalkyl), phenyl, or phenoxy.

In one embodiment, Ar¹ and Ar² are not ortho to each other (but may be meta or para, such as, for a five-membered ring they are 1,3 and for a 6-membered ring they are either 1,3 or 1,4).

In another embodiment Ar¹ is a substituted phenyl. This embodiment may be used in combination with the other embodiments of Het, Ar², R¹, R², R³, R⁴, Q¹, R² and R⁴ hydrocarbyl links, and/or L.

In another embodiment Ar¹ is a substituted phenyl that has one or more substituents selected from C₁-C₆ haloalkyl and C₁-C₆ haloalkoxy. This embodiment may be used in combination with the other embodiments of Het, Ar², R¹, R², R³, R⁴, Q¹, R² and R⁴ hydrocarbyl links, and/or L.

In another embodiment Ar¹ is a substituted phenyl that has one or more substituents selected from CN, SF₅, CH₃, CF₃, SCF₃, OCF₃, OCH₂CF₃, and OC₂F₅.

This embodiment may be used in combination with the other embodiments of Het, Ar², R¹, R², R³, R⁴, Q¹, R² and R⁴ hydrocarbyl links, and/or L.

In another embodiment Ar¹ is a substituted phenyl that has one or more substituents selected from CF₃, OCF₃, and OC₂F₅. This embodiment may be used in combination with the other embodiments of Het, Ar², R¹, R², R³, R⁴, Q¹, R² and R⁴ hydrocarbyl links, and/or L.

In another embodiment Ar¹ is a substituted pyridyl. This embodiment may be used in combination with the other embodiments of Het, Ar², R¹, R², R³, R⁴, Q¹, R² and R⁴ hydrocarbyl links, and/or L.

In another embodiment Ar¹ is a substituted pyridyl that has one or more substituents selected from C₁-C₆ haloalkyl and C₁-C₆ haloalkoxy. This embodiment may be used in combination with the other embodiments of Het, Ar², R¹, R², R³, R⁴, Q¹, R² and R⁴ hydrocarbyl links, and/or L.

In another embodiment Ar¹ is a substituted pyridinonyl. This embodiment may be used in combination with the other embodiments of Het, Ar², R¹, R², R³, R⁴, Q¹, R² and R⁴ hydrocarbyl links, and/or L.

In another embodiment Ar¹ is a substituted pyridinonyl that has one or more substituents selected from oxo, C₁-C₆ haloalkyl and C₁-C₆ haloalkoxy. This embodiment may be used in combination with the other embodiments of Het, Ar², R¹, R², R³, R⁴, Q¹, R² and R⁴ hydrocarbyl links, and/or L.

In another embodiment Het is selected from benzofuranyl, benzoisothiazolyl, benzoisoxazolyl, benzoxazolyl, benzothienyl, benzothiazolyl, cinnolinyl, furanyl, indazolyl, indolyl, imidazolyl, isoindolyl, isoquinolinyl, isothiazolyl, isoxazolyl, oxadiazolyl, oxazolinyl, oxazolyl, phthalazinyl, pyrazinyl, pyrazolinyl, pyrazolyl, pyridazinyl, pyridyl, pyrimidinyl, pyrrolyl, quinazolinyl, quinolinyl, quinoxalinyl, tetrazolyl, thiazolinyl, thiazolyl, thienyl, triazinyl, triazolyl, piperazinyl, piperidinyl, morpholinyl, pyrrolidinyl, tetrahydrofuranyl, tetrahydropyranyl, 1,2,3,4-tetrahydro-quinolinyl, 4,5-dihydro-oxazolyl, 4,5-dihydro-1H-pyrazolyl, 4,5-dihydro-isoxazolyl, and 2,3-dihydro-[1,3,4]-oxadiazolyl.

In another embodiment Het is selected from one of Het-A through Het-M. This embodiment may be used in combination with the other embodiments of Ar¹, Ar², R¹, R², R³, R⁴, Q¹, R² and R⁴ hydrocarbyl links, and/or L.

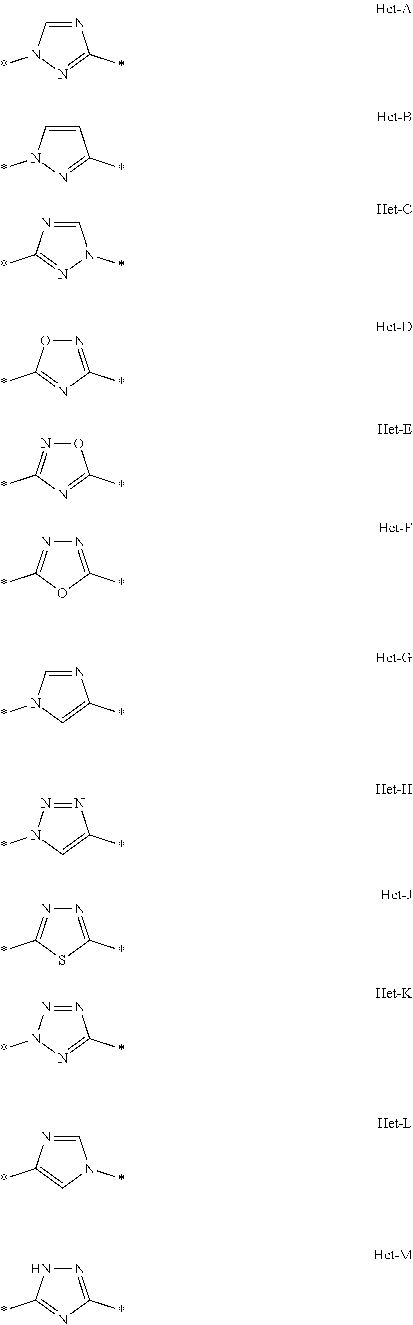

In another embodiment Het is triazolyl. This embodiment may be used in combination with the other embodiments of Ar¹, Ar², R¹, R², R³, R⁴, Q¹, R² and R⁴ hydrocarbyl links, and/or L.

In another embodiment Het is 1,2,4-triazolyl. This embodiment may be used in combination with the other embodiments of Ar¹, Ar², R¹, R², R³, R⁴, Q¹, R² and R⁴ hydrocarbyl links, and/or L.

In another embodiment Het is 1,2,3-triazolyl. This embodiment may be used in combination with the other embodiments of Ar¹, Ar², R¹, R², R³, R⁴, Q¹, R² and R⁴ hydrocarbyl links, and/or L.

In another embodiment Het is tetrazolyl. This embodiment may be used in combination with the other embodiments of Ar¹, Ar², R¹, R², R³, R⁴, Q¹, R² and R⁴ hydrocarbyl links, and/or L.

In another embodiment Het is 1,2,3,4-tetrazolyl. This embodiment may be used in combination with the other embodiments of Ar¹, Ar², R¹, R², R³, R⁴, Q¹, R² and R⁴ hydrocarbyl links, and/or L.

In another embodiment Het is oxadiazolyl. This embodiment may be used in combination with the other embodiments of Ar¹, Ar², R¹, R², R³, R⁴, Q¹, R² and R⁴ hydrocarbyl links, and/or L.

In another embodiment Het is 1,3,4-oxadiazolyl. This embodiment may be used in combination with the other embodiments of Ar¹, Ar², R¹, R², R³, R⁴, Q¹, R² and R⁴ hydrocarbyl links, and/or L.

In another embodiment Het is 1,2,4-oxadiazolyl. This embodiment may be used in combination with the other embodiments of Ar¹, Ar², R¹, R², R³, R⁴, Q¹, R² and R⁴ hydrocarbyl links, and/or L.

In another embodiment Het is thiadiazolyl. This embodiment may be used in combination with the other embodiments of Ar¹, Ar², R¹, R², R³, R⁴, Q¹, R² and R⁴ hydrocarbyl links, and/or L.

In another embodiment Het is 1,3,4-thiadiazolyl. This embodiment may be used in combination with the other embodiments of Ar¹, Ar², R¹, R², R³, R⁴, Q¹, R² and R⁴ hydrocarbyl links, and/or L.

In another embodiment Het is pyrazolyl. This embodiment may be used in combination with the other embodiments of Ar¹, Ar², R¹, R², R³, R⁴, Q¹, R² and R⁴ hydrocarbyl links, and/or L.

In another embodiment Het is imidazolyl. This embodiment may be used in combination with the other embodiments of Ar¹, Ar², R¹, R², R³, R⁴, Q¹, R² and R⁴ hydrocarbyl links, and/or L.

In another embodiment Ar² is phenyl. This embodiment may be used in combination with the other embodiments of Ar¹, Het, R¹, R², R³, R⁴, Q¹, R² and R⁴ hydrocarbyl links, and/or L.

In another embodiment Ar² is a substituted phenyl. This embodiment may be used in combination with the other embodiments of Ar¹, Het, R¹, R², R³, R⁴, Q¹, R² and R⁴ hydrocarbyl links, and/or L.

In another embodiment Ar² is a substituted phenyl that has one or more substituents selected from C₁-C₆ alkyl. This embodiment may be used in combination with the other embodiments of Ar¹, Het, R¹, R², R³, R⁴, Q¹, R² and R⁴ hydrocarbyl links, and/or L.

In another embodiment Ar² is a substituted phenyl that has one or more substituents wherein said substituent is H, F, Cl, Br, I, CN, OH, OCH₃, OCH₂OCH₃, CHF₂, CF₃, CH₂CH₃, CH(CH₃)₂, and CH₃. This embodiment may be used in combination with the other embodiments of Ar¹, Het, R¹, R², R³, R⁴, Q¹, R² and R⁴ hydrocarbyl links, and/or L.

In another embodiment Ar² is a substituted pyridyl. This embodiment may be used in combination with the other embodiments of Ar¹, Het, R¹, R², R³, R⁴, Q¹, R² and R⁴ hydrocarbyl links, and/or L.

In another embodiment Ar² is a substituted pyridyl that has one or more substituents selected from C₁-C₆ alkyl. This embodiment may be used in combination with the other embodiments of Ar¹, Het, R¹, R², R³, R⁴, Q¹, R² and R⁴ hydrocarbyl links, and/or L.

In another embodiment Ar² is a substituted phenyl that has one or more substituents wherein said substituent is CH₃. This embodiment may be used in combination with the other embodiments of Ar¹, Het, R¹, R², R³, R⁴, Q¹, R² and R⁴ hydrocarbyl links, and/or L.

In another embodiment R¹ is H. This embodiment may be used in combination with the other embodiments of Ar¹, Het, Ar², R², R³, R⁴, Q¹, R² and R⁴ hydrocarbyl links, and/or L.

In another embodiment R² is (J), H, C₁-C₆ alkyl, C₁-C₆ alkyl-O—C(═O)C₁-C₆ alkyl, C₁-C₆ alkyl-O—C(═O)N(R^{x}R^{y}), or (C₁-C₆ alkyl)S-(Het-1). This embodiment may be used in combination with the other embodiments of Ar¹, Het, Ar², R¹, R³, R⁴, Q¹, R² and R⁴ hydrocarbyl links, and/or L.

In another embodiment R² is (J), H, CH₃, C₁-C₆ alkyl, CH₂OC(═O)CH(CH₃)₂, CH₂OC(═O)N(H)(C(═O)OCH₂Ph), or CH₂S(3,4,5-trimethoxy-2-tetrahydropyran). This embodiment may be used in combination with the other embodiments of Ar¹, Het, Ar², R¹, R³, R⁴, Q¹, R² and R⁴ hydrocarbyl links, and/or L.

In another embodiment R³ is substituted phenyl. This embodiment may be used in combination with the other embodiments of Ar¹, Het, Ar², R¹, R², R⁴, Q¹, R² and R⁴ hydrocarbyl links, and/or L.

In another embodiment R³ is substituted phenyl wherein said substituted phenyl has one or more substituents selected from H, F, Cl, CN, OH, NO₂, NH₂, NHR^{x}, NR^{x}R^{y}, C₁-C₆ alkyl, C₃-C₆ cycloalkyl, C₁-C₆ alkoxy, C₁-C₆ haloalkoxy, S(C₁-C₆ alkyl), S(C₁-C₆ haloalkyl), C₁-C₆ haloalkyl, (C₁-C₆ alkyl)O(C₁-C₆ haloalkyl), (C₁-C₆ alkyl)O(C₁-C₆ alkyl), (C₁-C₆ alkyl)S(C₁-C₆ haloalkyl), (C₁-C₆ alkyl)S(═O)(C₁-C₆ haloalkyl), C₁-C₆ alkyl)S(═O)₂(C₁-C₆ haloalkyl), (C₁-C₆ alkyl)S(C₁-C₆ alkyl), (C₁-C₆ alkyl)S(═O)(C₁-C₆ alkyl), (C₁-C₆ alkyl)S(═O)₂(C₁-C₆ alkyl), C(═O)(C₁-C₆ alkyl), C(═O)O(C₁-C₆ alkyl), (Het-1), and phenyl. This embodiment may be used in combination with the other embodiments of Ar¹, Het, Ar², R¹, R², R⁴, Q¹, R² and R⁴ hydrocarbyl links, and/or L.

In another embodiment R³ is substituted phenyl wherein said substituted phenyl has one or more substituents selected from H, F, Cl, CN, NO₂, NH₂, NHCH₃, NHCH₂CH₃, NHCH₂CH₂CH₃, N(CH₃)₂, N(CH₂CH₃)₂, N(CH₂CH₂CH₃)₂, N(CH₃)C(═O)CH₃, N(CH₂CH₃)C(═O)CH₃, CH₃, CH₂CH₃, CH₂CH₂CH₃, CH₂CH₂CH₂CH₃, CF₃, CHF₂, CH₂CF₃, CF₂CH₃, CH(CH₃)₂, CH₂CH(CH₃)₂, CH(CH₃)(C₂H₅), OH, OCH₃, OCH₂CH₃, OCH₂CH₂CH₃, OCH₂CF₃, OCH₂CH₂CF₃, OCH₂CH₂CH₂CF₃, CH(CH₃)O(CH₃), CH₂OCH₃, CH₂OCH₂CH₃, CH₂OCH₂CF₃, OCH(CH₃)(CH₂CH₃), CH(CH₃)O(CH₂CH₃), CH(CF₃)O(CH₃), CH(CH₃)S(CH₃), C(═O)CH₃, C(═O)OCH₃, piperidinyl, and phenyl. This embodiment may be used in combination with the other embodiments of Ar¹, Het, Ar², R¹, R², R⁴, Q¹, R² and R⁴ hydrocarbyl links, and/or L.

In another embodiment R³ is substituted phenyl wherein said substituted phenyl has more than one substituent and at least one pair of said substituents are not ortho to each other. This embodiment may be used in combination with the other embodiments of Ar¹, Het, Ar², R¹, R², R⁴, Q¹, R² and R⁴ hydrocarbyl links, and/or L.

In another embodiment R³ is C₁-C₆ alkylphenyl. This embodiment may be used in combination with the other embodiments of Ar¹, Het, Ar², R¹, R², R⁴, Q¹, R² and R⁴ hydrocarbyl links, and/or L.

In another embodiment R³ is (Het-1). This embodiment may be used in combination with the other embodiments of Ar¹, Het, Ar², R¹, R², R⁴, Q¹, R² and R⁴ hydrocarbyl links, and/or L.

In another embodiment R⁴ is H. This embodiment may be used in combination with the other embodiments of Ar¹, Het, Ar², R¹, R², R³, Q¹, R² and R⁴ hydrocarbyl links, and/or L.

In another embodiment Q¹ is O. This embodiment may be used in combination with the other embodiments of Ar¹, Het, Ar², R¹, R², R³, R⁴, R² and R⁴ hydrocarbyl links, and/or L.

In another embodiment Q¹ is S. This embodiment may be used in combination with the other embodiments of Ar¹, Het, Ar², R¹, R², R³, R⁴, R² and R⁴ hydrocarbyl links, and/or L.

In another embodiment R² and R⁴ is a hydrocarbyl link wherein said hydrocarbyl link is substituted with oxo or C₁-C₆ alkyl. This embodiment may be used in combination with the other embodiments of Ar¹, Het, Ar², R¹, R², R³, R⁴, Q¹, and/or L.

In another embodiment R² and R⁴ is a hydrocarbyl link wherein said hydrocarbyl link is CH₂C(═O), C(C(OH)(CH₃)₂)C(═O), C(cyclopropyl)C(═O), C(CH₃)₂C(═O), CFHC(═O), CBrHC(═O), CH(CH₃)C(═O), CH₂CH₂, CH₂C(OH)(CH₃), CH₂CH₂CH₂, CH₂CH₂C(═O), CH₂CH(CH₃)CH₂, N(CH₃)C(═O), N(CH₂CH₃)C(═O), CH═C(CH₃), or CH₂CH(CH₃). This embodiment may be used in combination with the other embodiments of Ar¹, Het, Ar², R¹, R², R³, R⁴, Q¹, and/or L.

In another embodiment L is a bond, CH₂, CH₂CH₂, CH₂CH(CH₃), CH₂C(CH₃)₂, CH₂CH(CH₂CH₃), CH═CH, CH(CH₃)CH₂, C(CH₃)₂CH₂, CHBrCH₂, CH₂C(cyclopropyl), CH(CH₂CH₃)CH₂, C(CH₃)═CH, CH₂CH₂CH₂, CH(CH₃)CH(CH₃), CH₂CH₂CH₂CH₂, C≡CCH₂CH₂, cyclopropyl, or cyclohexyl. This embodiment may be used in combination with the other embodiments of Ar¹, Het, Ar², R¹, R², R³, R⁴, Q¹, and/or R² and R⁴ hydrocarbyl links.

In one embodiment, also provided are compound disclosed herein, wherein:

(A) Ar¹ is a substituted phenyl, a substituted pyridazinyl, a substituted pyridyl, a substituted pyridinonyl, or a substituted pyrimidinyl, wherein said substituted phenyl, substituted pyridyl, and substituted pyrimidinyl have one or more substituents independently selected from H, CN, SF₅, oxo, C₁-C₆ alkyl, C₁-C₆ alkoxy, S(C₁-C₆ haloalkyl), C₁-C₆ haloalkyl and C₁-C₆ haloalkoxy;
(B) Het is selected from one of Het-A through Het-M

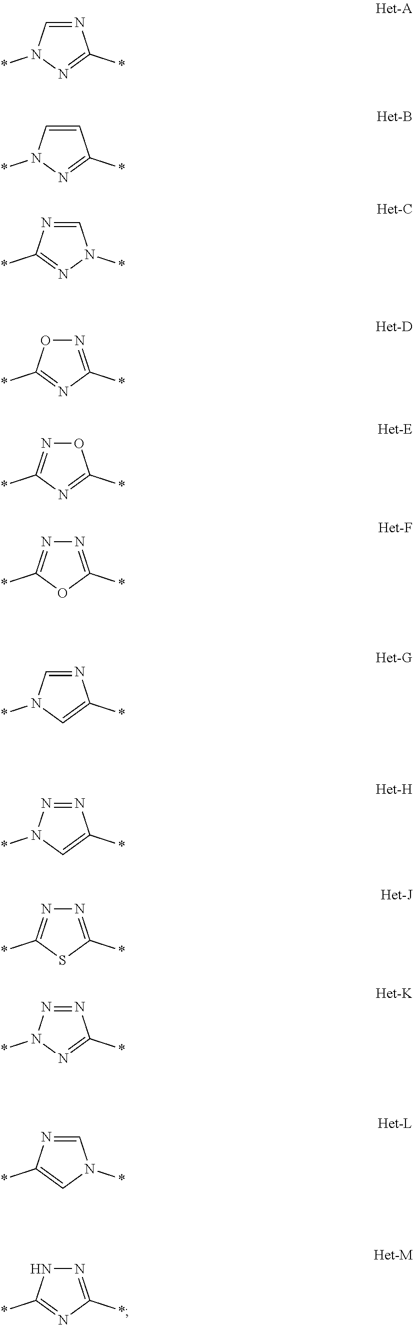

(C) Ar² is phenyl, pyridyl, pyrimidinyl, substituted phenyl, substituted pyridyl, or substituted pyrimidinyl, wherein said substituted phenyl, substituted pyridyl, and substituted pyrimidinyl have one or more substituents independently selected from H, F, Cl, Br, I, CN, NO₂, NR^{x}R^{y}, OH, C₁-C₆ alkoxy, (C₁-C₆ alkyl)O(C₁-C₆ alkyl), C₁-C₆ haloalkyl, and C₁-C₆ alkyl;
(D) R¹ is H, C₁-C₆ alkyl, C₃-C₆ cycloalkyl, or C₂-C₆ alkenyl, wherein said alkyl, cycloalkyl, or alkenyl is optionally substituted with a C₁-C₆ alkoxy, C₁-C₆ haloalkoxy, C₃-C₆ cycloalkyl, or C₃-C₆ halocycloalkyl;
(E) R² is (J), H, C₁-C₆ alkyl, or C₁-C₆ haloalkyl;
(F) R³ is a substituted phenyl, wherein said substituted phenyl has a substituent selected from N(CH₃)C(═O)CH₃, N(CH₂CH₃)C(═O)CH₃, OH, SH, S(C₁-C₆ alkyl), S(C₁-C₆ haloalkyl), C₁-C₆ haloalkyl, (C₁-C₆ alkyl)S(C₁-C₆ alkyl), (C₁-C₆ alkyl)S(C₁-C₆ haloalkyl), (C₁-C₆ alkyl)O(C₁-C₆ haloalkyl), (C₁-C₈ haloalkyl)O(C₁-C₈ alkyl), or (C₁-C₆ alkyl)O(C₁-C₆ alkyl); and

said substituted phenyl is optionally substituted with one or more substituents independently selected from H, F, Cl, Br, I, CN, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ cycloalkyl, C₁-C₆ halocycloalkyl, C₁-C₆ alkoxy, C₁-C₆ haloalkoxy, phenyl, C(═O)(C₁-C₆ alkyl), C(═O)O(C₁-C₆ alkyl), (C₁-C₈ alkyl)S(C₁-C₈ alkyl), or (C₁-C₈ alkyl)O(C₁-C₈ alkyl);

(G) R⁴ is selected from (J), H, C₁-C₆ alkyl, or C₁-C₆ haloalkyl;
(H) Q¹ is S and Q² is O;
(I) R^{x} and R^{y} are independently selected from H, C(═O)(C₁-C₆ alkyl), C₁-C₆ alkyl, C₁-C₆ haloalkyl, C₃-C₆ cycloalkyl, C₃-C₆ halocycloalkyl, C₂-C₆ alkenyl, C₃-C₆ cycloalkenyl, C₂-C₆ haloalkenyl, C₂-C₆ alkynyl, and phenyl;
(J) R² and R⁴ may be a 1- to 4-membered saturated or unsaturated, hydrocarbyl link, which may contain one or more heteroatoms selected from nitrogen, sulfur, and oxygen, and together with C^{x}(Q¹)(N^{x}) forms a cyclic structure, wherein said hydrocarbyl link may optionally be substituted with one or more substituents independently selected from R⁵, R⁶, and R⁷, wherein each R⁵, R⁶, and R⁷ is selected from H, F, Cl, Br, I, CN, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ alkoxy, C₁-C₆ haloalkoxy, C₃-C₆ cycloalkyl, C₃-C₆ halocycloalkyl, phenyl, and oxo; and
(L) L is a linker selected from

(1) a bond,

(2) a saturated or unsaturated, substituted or unsubstituted, linear (C₁-C₄)hydrocarbyl linker, or

(3) a saturated or unsaturated, substituted or unsubstituted, cyclic (C₃-C₈)hydrocarbyl group linker,

wherein each of said linkers connects Ar² to N^{Y} and

wherein said substituted linear (C₁-C₄)hydrocarbyl linker and substituted cyclic (C₃-C₈)hydrocarbyl linker has one or more substituents independently selected from R⁸, R⁹, R¹⁰, R¹¹, and R¹², wherein each R⁸, R⁹, R¹⁰, R¹¹, and R¹², is selected from H, F, Cl, Br, I, CN, oxo, thioxo, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ alkoxy, C₁-C₆ haloalkoxy, C₂-C₆ alkenyl, C₂-C₆ haloalkenyl, C₂-C₆ alkynyl, C₃-C₆ cycloalkyl, C₃-C₆ halocycloalkyl, C₃-C₆ cycloalkenyl, C₃-C₆ halocycloalkenyl, or phenyl.

In another embodiment, the compound provided herein has a construction of the following formula (“Formula Two”):

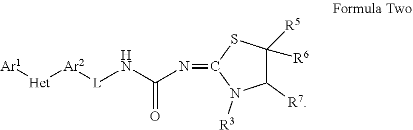

In another embodiment, the compound provided herein has a structure selected from compounds listed in Table 1 and Table 1A, 1B, and 1C.

In another embodiment, the compound provided herein has the structure of Formula Two and selected from compounds listed in Table 1 and the group consisting of H1, H2, H3, H4, H6, H7, H8, H10, H11, H12, H13, H14, H15, H16, H17, H20, and H21.

In another embodiment, the compound provided herein has the structure of Formulae Three, Four and/or Five and selected from compounds listed in Tables 1A, 1B, and/or 1C and the group consisting of J30, J34, J39, J42, J49, J50, J60, J62, J66, J67, J72, J74, J81, J87, J88, J90, J94, J97, J117, J119, J120, J123, J124, J139, J141, J165, J167, J168, J173, J174, J178, J179, J180, J181, J182, J185, J186, J187, J188, J189, J190, J194, J195, J197, J199, J200, J203, J204, J205, J206, J214, J215, J220, J221, J222, J226, J228, J229, J230, J232, J233, J234, J236, J237, J240, J241, J244, J245, J249, J250, J256, J262, J265, J267, J271, J274, J276, J280, J281, J286, J289, J291, J297, J298, J299, J300, J303, J304, J306, J308, J309, J310, and J311. In another embodiment, the compound provided is selected from the group consisting of J30, J34, J39, J42, J49, J50, J60, J62, J66, J67, J72, J74, J81, J87, J88, J90, J94, J97, J117, J119, J120, J123, J124, J139, J141, J165, J167, J168, J173, J174, J178, J179, J180, J181, J182, J185, J186, J187, J188, J189, J190, J194, J195, J197, J199, J200, J203, J204, J205, J206, J214, J215, J220, J221, J222, J226, J228, J229, J230, J232, J233, J234, J236, J237, J240, J241, J244, J245, J249, J250, J256, J262, J265, J267, J271, J274, J276, J280, J281, J286, J289, J291, J297, J298, J299, J300, J303, J304, J306, J308, J309, J310, J311, J312, J314, J315, A16, J318, J319, J324, J325, J326, J327, J328, J344, J350, J362, J363, J364, J365, J366, J368, J510, J539, J570, J574, J645, J646, J647, J648, J649, J650, J652, J653, J654, J655, J656, J657, J659, J663, J684, J686, J716, J719, J742, J745, J747, J751, J778, J779, 780, J781, J782, J783, J784, J785, J786, J787, J788, J789, J790, J791, J792, J793, J794, J795, J796, J797, J798, and J799.

In another embodiment, the compound provided herein has the structure selected from compounds listed in Table 3.

TABLE 3

| | |
|---|---|
| H1 | 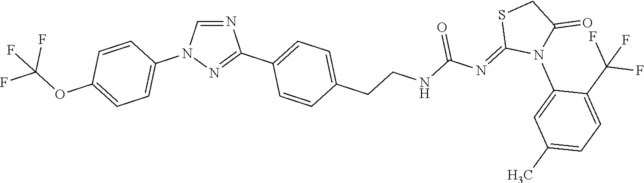 [SMILES: Cc1ccc(C(F)(F)F)c(N2C(=O)CS/C2=N\C(=O)NCCc2ccc(-c3ncn(-c4ccc(OC(F)(F)F)cc4)n3)cc2)c1] |
| H2 | 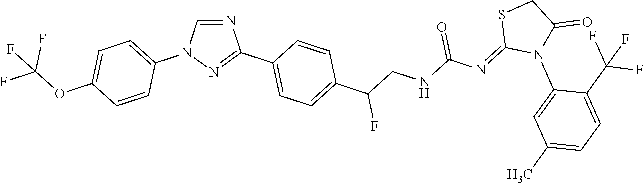 [SMILES: Cc1ccc(C(F)(F)F)c(N2C(=O)CS/C2=N\C(=O)NCC(F)c2ccc(-c3ncn(-c4ccc(OC(F)(F)F)cc4)n3)cc2)c1] |
| H3 | 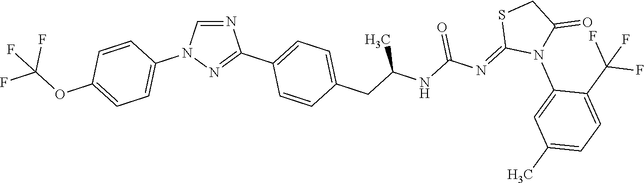 [SMILES: Cc1ccc(C(F)(F)F)c(N2C(=O)CS/C2=N\C(=O)N[C@H](C)Cc2ccc(-c3ncn(-c4ccc(OC(F)(F)F)cc4)n3)cc2)c1] |
| H4 | 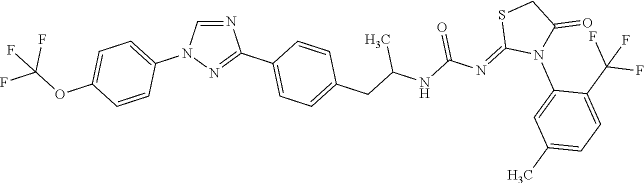 [SMILES: Cc1ccc(C(F)(F)F)c(N2C(=O)CS/C2=N\C(=O)NC(C)Cc2ccc(-c3ncn(-c4ccc(OC(F)(F)F)cc4)n3)cc2)c1] |
| H6 | 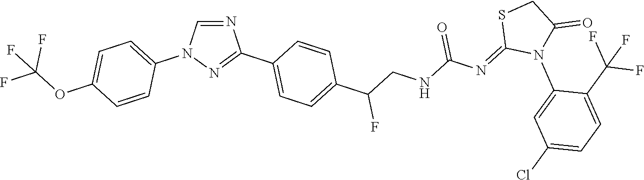 [SMILES: O=C(/N=C1\SCC(=O)N1c1cc(Cl)ccc1C(F)(F)F)NCC(F)c1ccc(-c2ncn(-c3ccc(OC(F)(F)F)cc3)n2)cc1] |
| H7 | 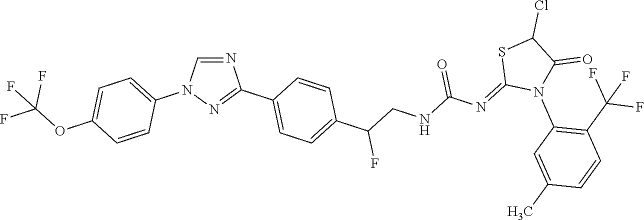 [SMILES: Cc1ccc(C(F)(F)F)c(N2C(=O)C(Cl)S/C2=N\C(=O)NCC(F)c2ccc(-c3ncn(-c4ccc(OC(F)(F)F)cc4)n3)cc2)c1] |
| H8 | 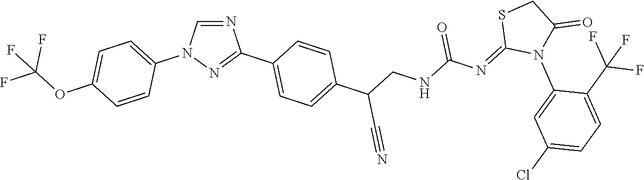 [SMILES: N#CC(CNC(=O)/N=C1\SCC(=O)N1c1cc(Cl)ccc1C(F)(F)F)c1ccc(-c2ncn(-c3ccc(OC(F)(F)F)cc3)n2)cc1] |
| H10 | 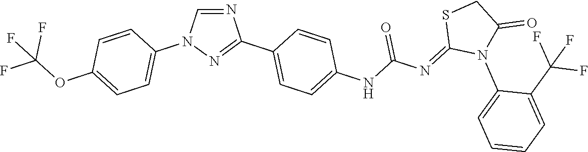 [SMILES: O=C(/N=C1\SCC(=O)N1c1ccccc1C(F)(F)F)Nc1ccc(-c2ncn(-c3ccc(OC(F)(F)F)cc3)n2)cc1] |
| H11 | 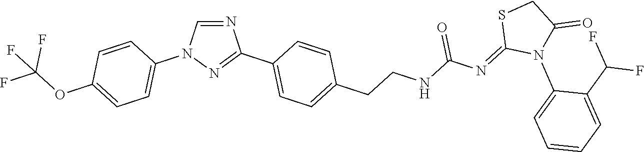 [SMILES: O=C(/N=C1\SCC(=O)N1c1ccccc1C(F)F)NCCc1ccc(-c2ncn(-c3ccc(OC(F)(F)F)cc3)n2)cc1] |
| H12 | 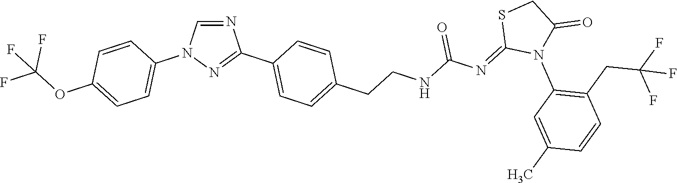 [SMILES: Cc1ccc(CC(F)(F)F)c(N2C(=O)CS/C2=N\C(=O)NCCc2ccc(-c3ncn(-c4ccc(OC(F)(F)F)cc4)n3)cc2)c1] |
| H13 | 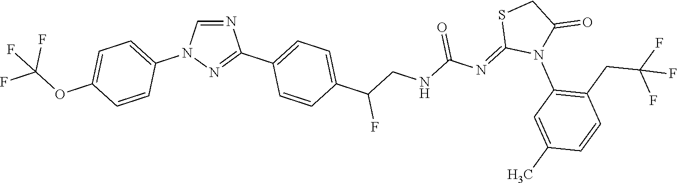 [SMILES: Cc1ccc(CC(F)(F)F)c(N2C(=O)CS/C2=N\C(=O)NCC(F)c2ccc(-c3ncn(-c4ccc(OC(F)(F)F)cc4)n3)cc2)c1] |
| H14 | 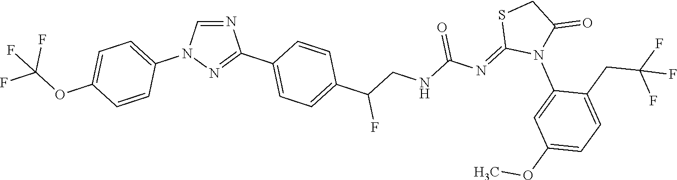 [SMILES: COc1ccc(CC(F)(F)F)c(N2C(=O)CS/C2=N\C(=O)NCC(F)c2ccc(-c3ncn(-c4ccc(OC(F)(F)F)cc4)n3)cc2)c1] |
| H15 | 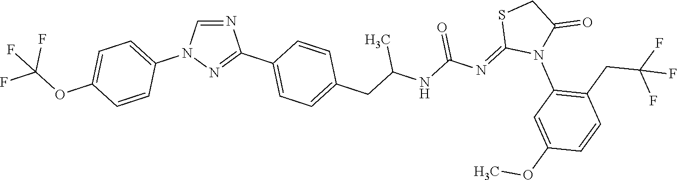 [SMILES: COc1ccc(CC(F)(F)F)c(N2C(=O)CS/C2=N\C(=O)NC(C)Cc2ccc(-c3ncn(-c4ccc(OC(F)(F)F)cc4)n3)cc2)c1] |
| H16 | 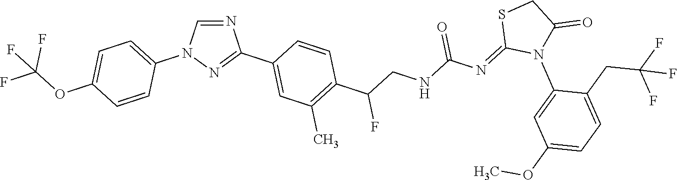 [SMILES: COc1ccc(CC(F)(F)F)c(N2C(=O)CS/C2=N\C(=O)NCC(F)c2ccc(-c3ncn(-c4ccc(OC(F)(F)F)cc4)n3)cc2C)c1] |
| H17 | 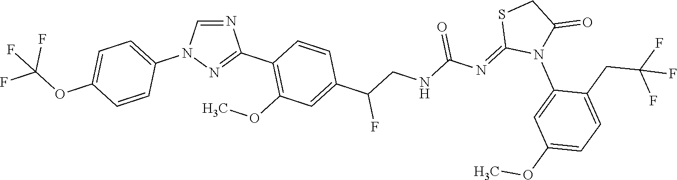 [SMILES: COc1ccc(CC(F)(F)F)c(N2C(=O)CS/C2=N\C(=O)NCC(F)c2ccc(-c3ncn(-c4ccc(OC(F)(F)F)cc4)n3)c(OC)c2)c1] |
| H20 | 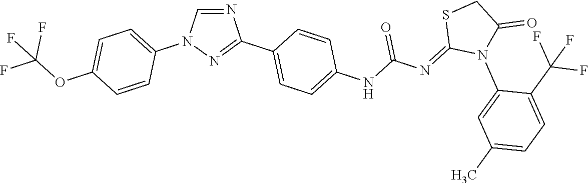 [SMILES: Cc1ccc(C(F)(F)F)c(N2C(=O)CS/C2=N\C(=O)Nc2ccc(-c3ncn(-c4ccc(OC(F)(F)F)cc4)n3)cc2)c1] |
| H21 | 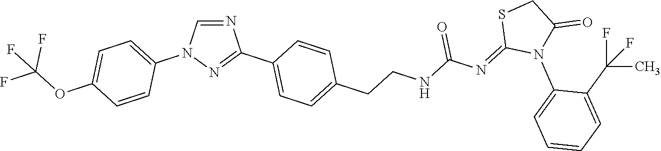 [SMILES: CC(F)(F)c1ccccc1N1C(=O)CS/C1=N\C(=O)NCCc1ccc(-c2ncn(-c3ccc(OC(F)(F)F)cc3)n2)cc1] |
| J1 | 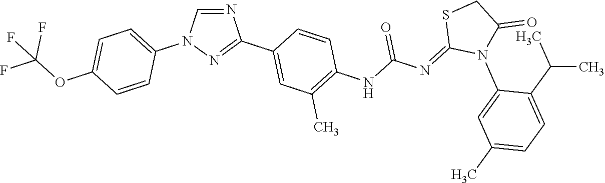 [SMILES: Cc1ccc(C(C)C)c(N2C(=O)CS/C2=N\C(=O)Nc2ccc(-c3ncn(-c4ccc(OC(F)(F)F)cc4)n3)cc2C)c1] |
| J2 | 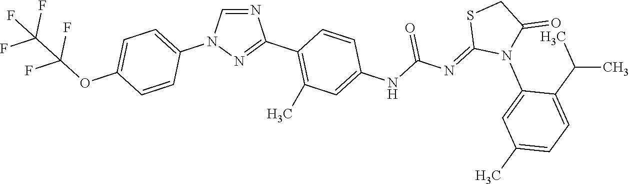 [SMILES: Cc1ccc(C(C)C)c(N2C(=O)CS/C2=N\C(=O)Nc2ccc(-c3ncn(-c4ccc(OC(F)(F)C(F)(F)F)cc4)n3)c(C)c2)c1] |
| J4 | 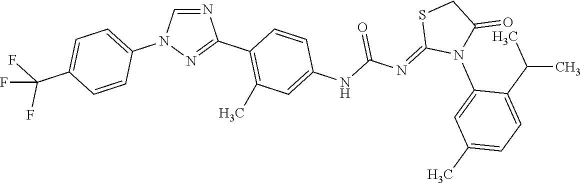 [SMILES: Cc1ccc(C(C)C)c(N2C(=O)CS/C2=N\C(=O)Nc2ccc(-c3ncn(-c4ccc(C(F)(F)F)cc4)n3)c(C)c2)c1] |
| J7 | 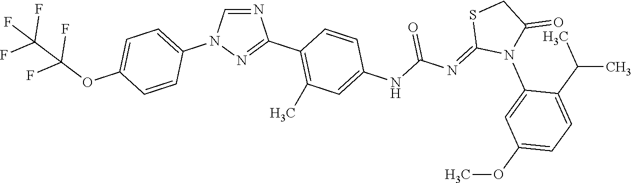 [SMILES: COc1ccc(C(C)C)c(N2C(=O)CS/C2=N\C(=O)Nc2ccc(-c3ncn(-c4ccc(OC(F)(F)C(F)(F)F)cc4)n3)c(C)c2)c1] |
| J10 | 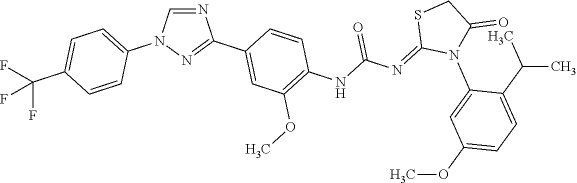 [SMILES: COc1ccc(C(C)C)c(N2C(=O)CS/C2=N\C(=O)Nc2ccc(-c3ncn(-c4ccc(C(F)(F)F)cc4)n3)cc2OC)c1] |
| J12 | 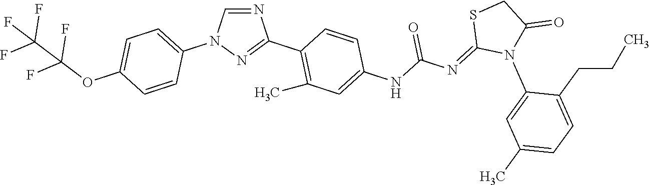 [SMILES: CCCc1ccc(C)cc1N1C(=O)CS/C1=N\C(=O)Nc1ccc(-c2ncn(-c3ccc(OC(F)(F)C(F)(F)F)cc3)n2)c(C)c1] |
| J13 | 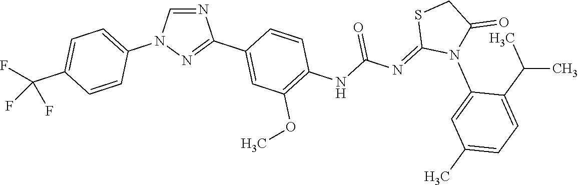 [SMILES: COc1cc(-c2ncn(-c3ccc(C(F)(F)F)cc3)n2)ccc1NC(=O)/N=C1\SCC(=O)N1c1cc(C)ccc1C(C)C] |
| J15 | 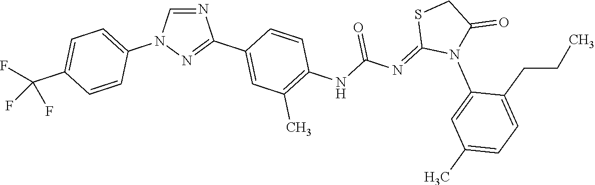 [SMILES: CCCc1ccc(C)cc1N1C(=O)CS/C1=N\C(=O)Nc1ccc(-c2ncn(-c3ccc(C(F)(F)F)cc3)n2)cc1C] |
| J19 | 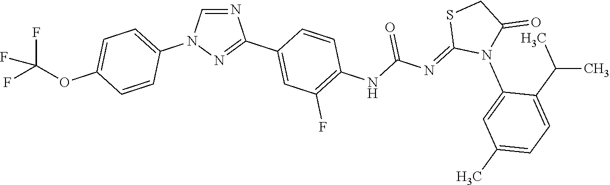 [SMILES: Cc1ccc(C(C)C)c(N2C(=O)CS/C2=N\C(=O)Nc2ccc(-c3ncn(-c4ccc(OC(F)(F)F)cc4)n3)cc2F)c1] |
| J21 | 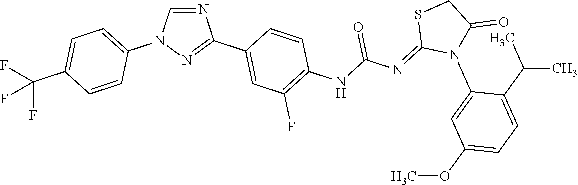 [SMILES: COc1ccc(C(C)C)c(N2C(=O)CS/C2=N\C(=O)Nc2ccc(-c3ncn(-c4ccc(C(F)(F)F)cc4)n3)cc2F)c1] |
| J24 | 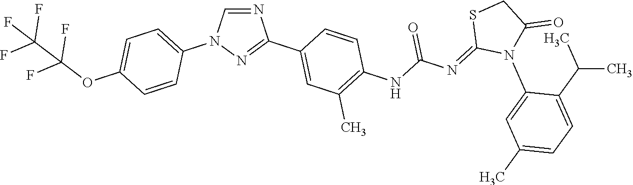 [SMILES: Cc1ccc(C(C)C)c(N2C(=O)CS/C2=N\C(=O)Nc2ccc(-c3ncn(-c4ccc(OC(F)(F)C(F)(F)F)cc4)n3)cc2C)c1] |
| J26 | 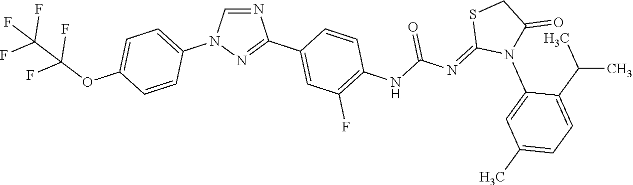 [SMILES: Cc1ccc(C(C)C)c(N2C(=O)CS/C2=N\C(=O)Nc2ccc(-c3ncn(-c4ccc(OC(F)(F)C(F)(F)F)cc4)n3)cc2F)c1] |
| J30 | 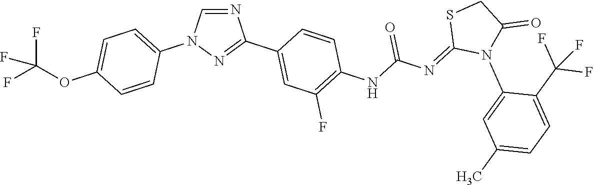 [SMILES: Cc1ccc(C(F)(F)F)c(N2C(=O)CS/C2=N\C(=O)Nc2ccc(-c3ncn(-c4ccc(OC(F)(F)F)cc4)n3)cc2F)c1] |
| J32 | 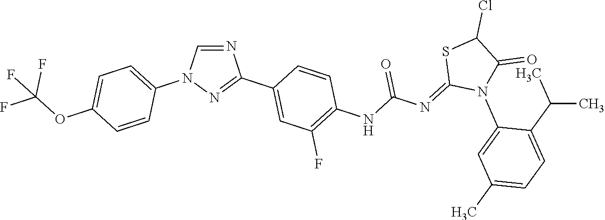 [SMILES: Cc1ccc(C(C)C)c(N2C(=O)C(Cl)S/C2=N\C(=O)Nc2ccc(-c3ncn(-c4ccc(OC(F)(F)F)cc4)n3)cc2F)c1] |
| J35 | 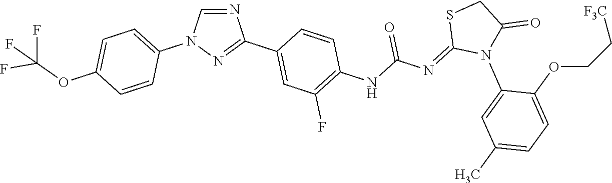 [SMILES: Cc1ccc(OCCC(F)(F)F)c(N2C(=O)CS/C2=N\C(=O)Nc2ccc(-c3ncn(-c4ccc(OC(F)(F)F)cc4)n3)cc2F)c1] |
| J38 | 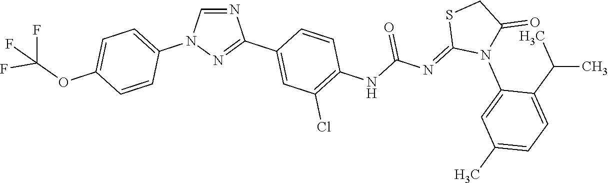 [SMILES: Cc1ccc(C(C)C)c(N2C(=O)CS/C2=N\C(=O)Nc2ccc(-c3ncn(-c4ccc(OC(F)(F)F)cc4)n3)cc2Cl)c1] |
| J39 | 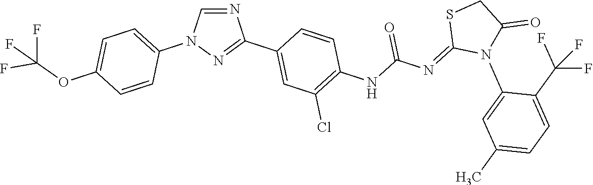 [SMILES: Cc1ccc(C(F)(F)F)c(N2C(=O)CS/C2=N\C(=O)Nc2ccc(-c3ncn(-c4ccc(OC(F)(F)F)cc4)n3)cc2Cl)c1] |
| J42 | 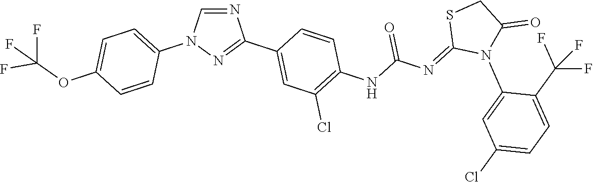 [SMILES: O=C(/N=C1\SCC(=O)N1c1cc(Cl)ccc1C(F)(F)F)Nc1ccc(-c2ncn(-c3ccc(OC(F)(F)F)cc3)n2)cc1Cl] |
| J49 | 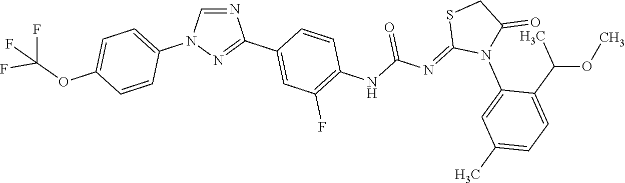 [SMILES: COC(C)c1ccc(C)cc1N1C(=O)CS/C1=N\C(=O)Nc1ccc(-c2ncn(-c3ccc(OC(F)(F)F)cc3)n2)cc1F] |
| J53 | 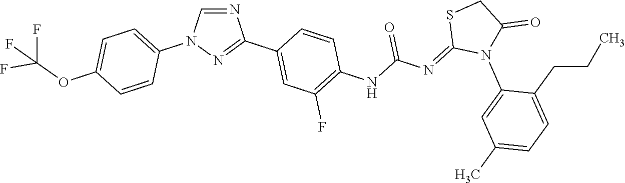 [SMILES: CCCc1ccc(C)cc1N1C(=O)CS/C1=N\C(=O)Nc1ccc(-c2ncn(-c3ccc(OC(F)(F)F)cc3)n2)cc1F] |
| J54 | 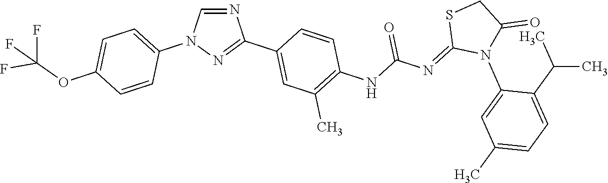 [SMILES: Cc1ccc(C(C)C)c(N2C(=O)CS/C2=N\C(=O)Nc2ccc(-c3ncn(-c4ccc(OC(F)(F)F)cc4)n3)cc2C)c1] |
| J57 | 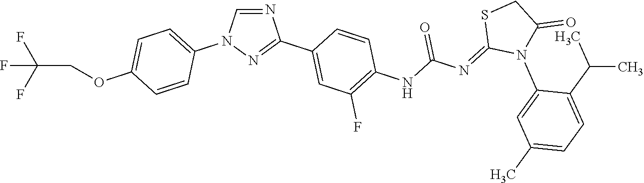 [SMILES: Cc1ccc(C(C)C)c(N2C(=O)CS/C2=N\C(=O)Nc2ccc(-c3ncn(-c4ccc(OCC(F)(F)F)cc4)n3)cc2F)c1] |
| J62 | 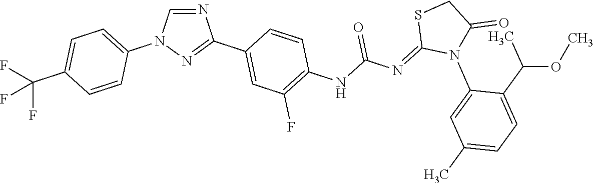 [SMILES: COC(C)c1ccc(C)cc1N1C(=O)CS/C1=N\C(=O)Nc1ccc(-c2ncn(-c3ccc(C(F)(F)F)cc3)n2)cc1F] |
| J94 | 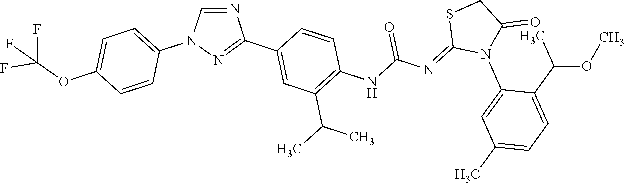 [SMILES: COC(C)c1ccc(C)cc1N1C(=O)CS/C1=N\C(=O)Nc1ccc(-c2ncn(-c3ccc(OC(F)(F)F)cc3)n2)cc1C(C)C] |
| J96 | 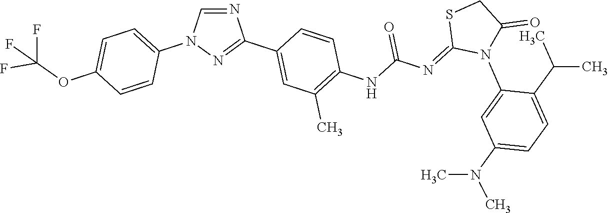 [SMILES: Cc1cc(-c2ncn(-c3ccc(OC(F)(F)F)cc3)n2)ccc1NC(=O)/N=C1\SCC(=O)N1c1cc(N(C)C)ccc1C(C)C] |
| J109 | 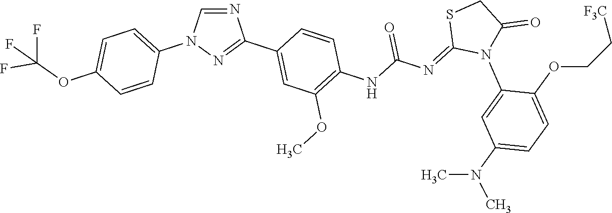 [SMILES: COc1cc(-c2ncn(-c3ccc(OC(F)(F)F)cc3)n2)ccc1NC(=O)/N=C1\SCC(=O)N1c1cc(N(C)C)ccc1OCCC(F)(F)F] |
| J110 | 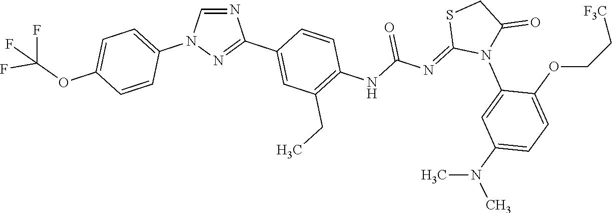 [SMILES: CCc1cc(-c2ncn(-c3ccc(OC(F)(F)F)cc3)n2)ccc1NC(=O)/N=C1\SCC(=O)N1c1cc(N(C)C)ccc1OCCC(F)(F)F] |
| J111 | 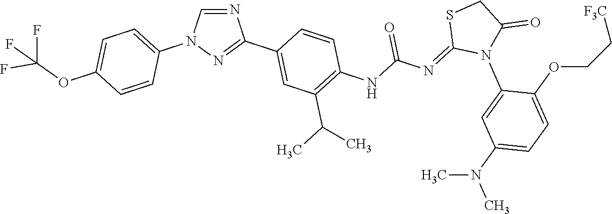 [SMILES: CC(C)c1cc(-c2ncn(-c3ccc(OC(F)(F)F)cc3)n2)ccc1NC(=O)/N=C1\SCC(=O)N1c1cc(N(C)C)ccc1OCCC(F)(F)F] |
| J119 | 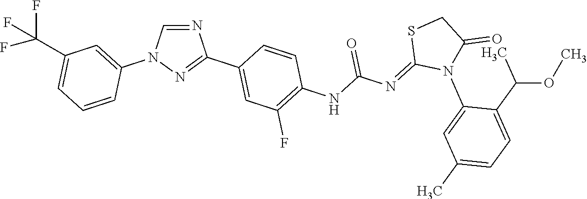 [SMILES: COC(C)c1ccc(C)cc1N1C(=O)CS/C1=N\C(=O)Nc1ccc(-c2ncn(-c3cccc(C(F)(F)F)c3)n2)cc1F] |
| J123 | 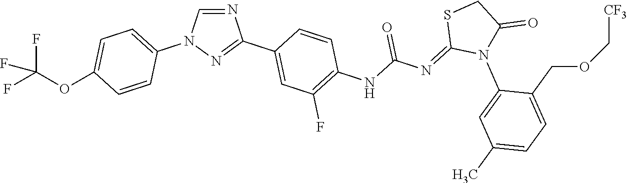 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2F)c1] |
| J124 | 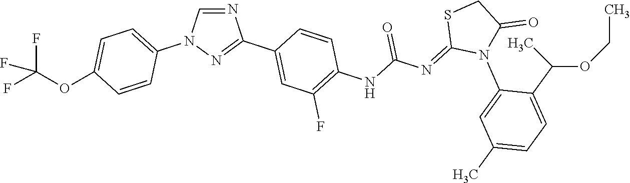 [SMILES: CCOC(C)c1ccc(C)cc1N1C(=O)CS/C1=N\C(=O)Nc1ccc(-c2ncn(-c3ccc(OC(F)(F)F)cc3)n2)cc1F] |
| J139 | 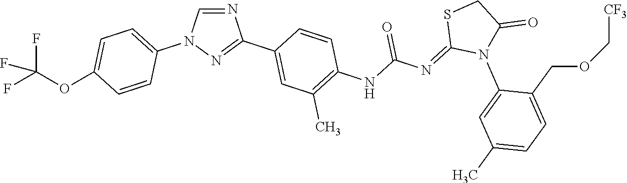 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2C)c1] |
| J163 | 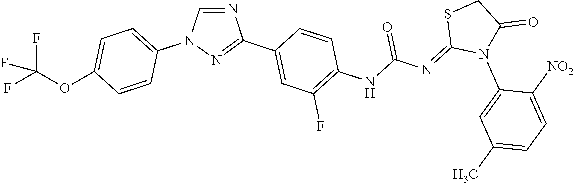 [SMILES: Cc1ccc([N+](=O)[O-])c(N2C(=O)CS/C2=N\C(=O)Nc2ccc(-c3ncn(-c4ccc(OC(F)(F)F)cc4)n3)cc2F)c1] |
| J181 | 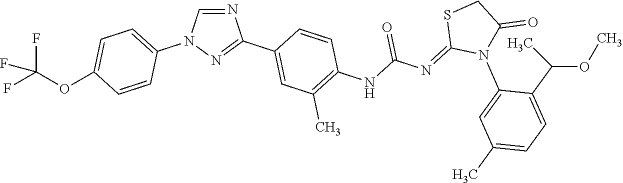 [SMILES: COC(C)c1ccc(C)cc1N1C(=O)CS/C1=N\C(=O)Nc1ccc(-c2ncn(-c3ccc(OC(F)(F)F)cc3)n2)cc1C] |
| J186 | 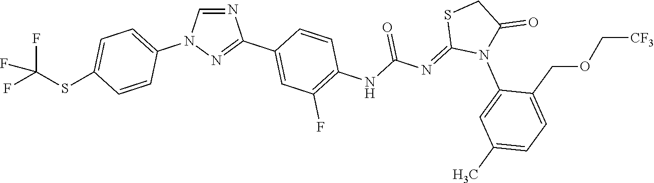 [SMILES: Cc1ccc(COCC(F)(F)F)c(N2C(=O)CS/C2=N\C(=O)Nc2ccc(-c3ncn(-c4ccc(SC(F)(F)F)cc4)n3)cc2F)c1] |
| J187 | 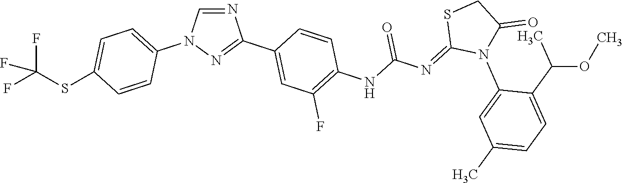 [SMILES: COC(C)c1ccc(C)cc1N1C(=O)CS/C1=N\C(=O)Nc1ccc(-c2ncn(-c3ccc(SC(F)(F)F)cc3)n2)cc1F] |
| J194 | 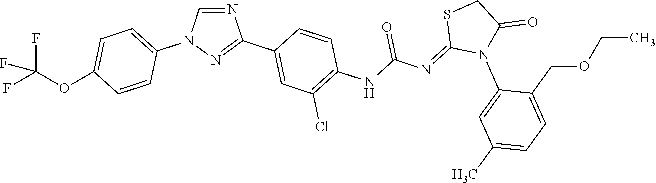 [SMILES: CCOCc1ccc(C)cc1N1C(=O)CS/C1=N\C(=O)Nc1ccc(-c2ncn(-c3ccc(OC(F)(F)F)cc3)n2)cc1Cl] |
| J214 | 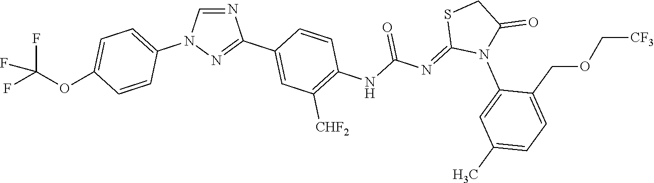 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2C(F)F)c1] |
| J215 | 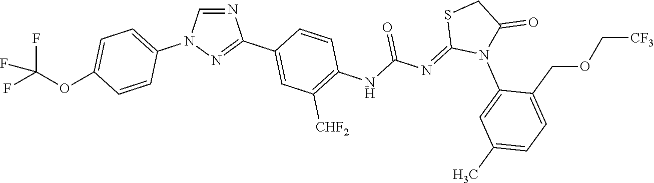 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2C(F)F)c1] |
| J217 | 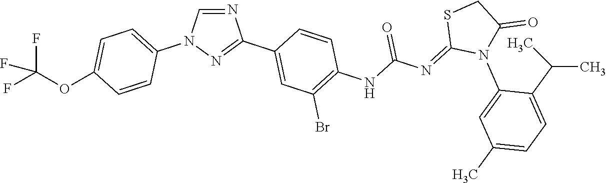 [SMILES: Cc1ccc(C(C)C)c(N2C(=O)CS/C2=N\C(=O)Nc2ccc(-c3ncn(-c4ccc(OC(F)(F)F)cc4)n3)cc2Br)c1] |
| J221 | 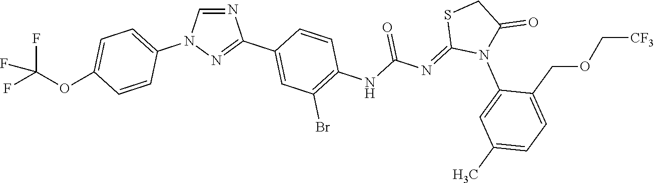 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2Br)c1] |
| J240 | 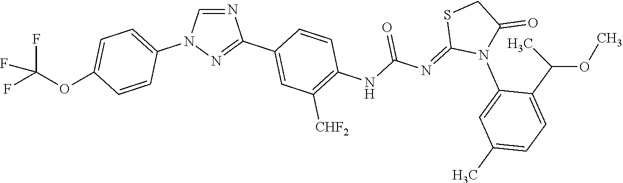 [SMILES: COC(C)c1ccc(C)cc1N1C(=O)CS/C1=N\C(=O)Nc1ccc(-c2ncn(-c3ccc(OC(F)(F)F)cc3)n2)cc1C(F)F] |
| J251 | 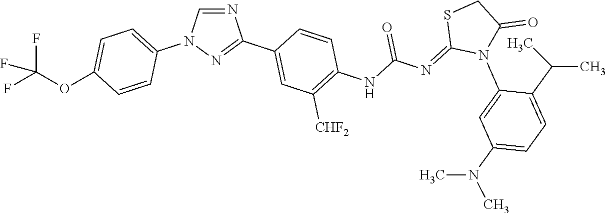 [SMILES: CC(C)c1ccc(N(C)C)cc1N1C(=O)CS/C1=N\C(=O)Nc1ccc(-c2ncn(-c3ccc(OC(F)(F)F)cc3)n2)cc1C(F)F] |
| J257 | 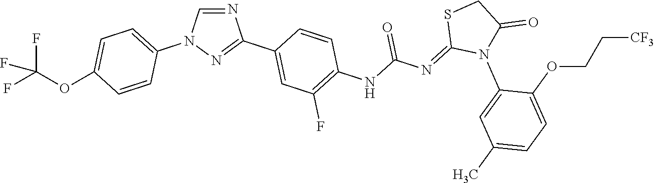 [SMILES: Cc1ccc(OCCC(F)(F)F)c(N2C(=O)CS/C2=N\C(=O)Nc2ccc(-c3ncn(-c4ccc(OC(F)(F)F)cc4)n3)cc2F)c1] |
| J259 | 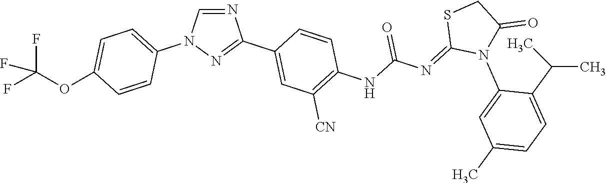 [SMILES: Cc1ccc(C(C)C)c(N2C(=O)CS/C2=N\C(=O)Nc2ccc(-c3ncn(-c4ccc(OC(F)(F)F)cc4)n3)cc2C#N)c1] |
| J264 | 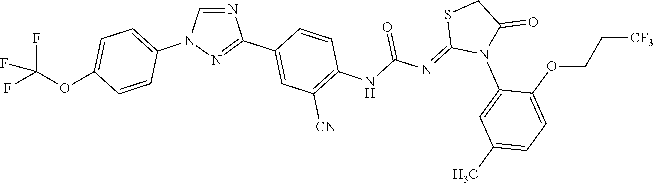 [SMILES: Cc1ccc(OCCC(F)(F)F)c(N2C(=O)CS/C2=N\C(=O)Nc2ccc(-c3ncn(-c4ccc(OC(F)(F)F)cc4)n3)cc2C#N)c1] |
| J266 | 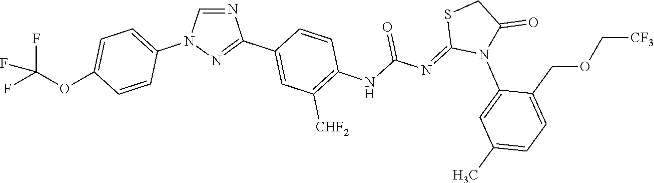 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2C(F)F)c1] |
| J287 | 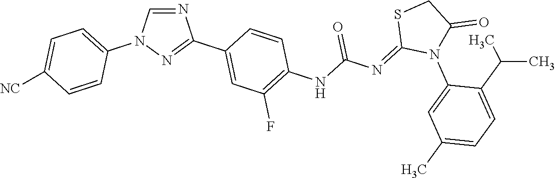 [SMILES: Cc1ccc(C(C)C)c(N2C(=O)CS/C2=N\C(=O)Nc2ccc(-c3ncn(-c4ccc(C#N)cc4)n3)cc2F)c1] |
| J307 | 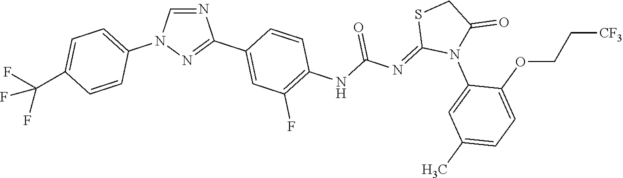 [SMILES: Cc1ccc(OCCC(F)(F)F)c(N2C(=O)CS/C2=N\C(=O)Nc2ccc(-c3ncn(-c4ccc(C(F)(F)F)cc4)n3)cc2F)c1] |
| J309 | 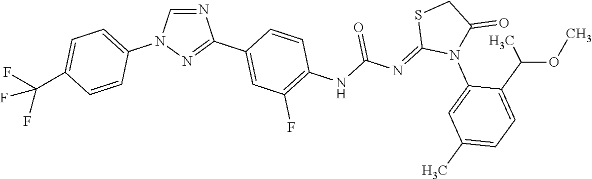 [SMILES: COC(C)c1ccc(C)cc1N1C(=O)CS/C1=N\C(=O)Nc1ccc(-c2ncn(-c3ccc(C(F)(F)F)cc3)n2)cc1F] |
| J312 | 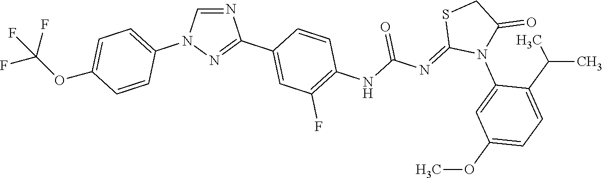 [SMILES: COc1ccc(C(C)C)c(N2C(=O)CS/C2=N\C(=O)Nc2ccc(-c3ncn(-c4ccc(OC(F)(F)F)cc4)n3)cc2F)c1] |
| J313 | 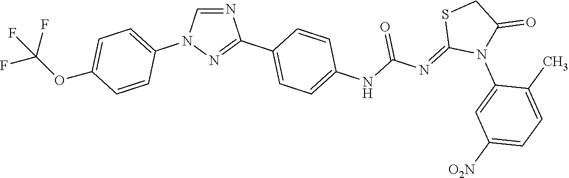 [SMILES: Cc1ccc([N+](=O)[O-])cc1N1C(=O)CS/C1=N\C(=O)Nc1ccc(-c2ncn(-c3ccc(OC(F)(F)F)cc3)n2)cc1] |
| J314 | 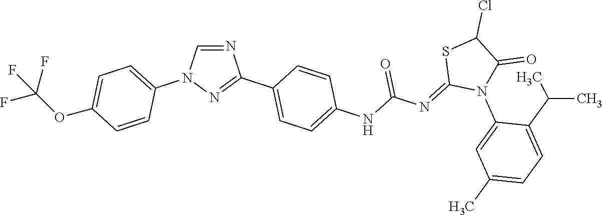 [SMILES: Cc1ccc(C(C)C)c(N2C(=O)C(Cl)S/C2=N\C(=O)Nc2ccc(-c3ncn(-c4ccc(OC(F)(F)F)cc4)n3)cc2)c1] |
| J315 | 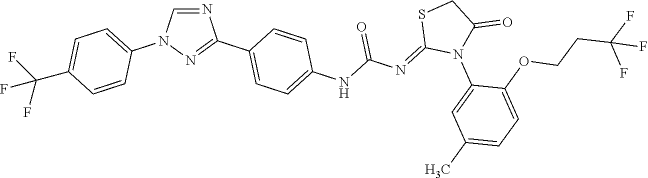 [SMILES: Cc1ccc(OCCC(F)(F)F)c(N2C(=O)CS/C2=N\C(=O)Nc2ccc(-c3ncn(-c4ccc(C(F)(F)F)cc4)n3)cc2)c1] |
| J316 | 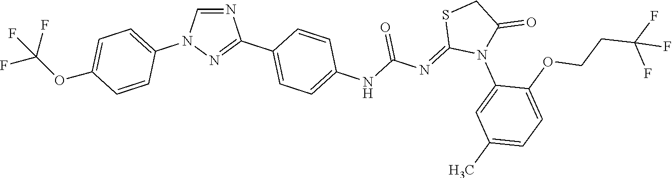 [SMILES: Cc1ccc(OCCC(F)(F)F)c(N2C(=O)CS/C2=N\C(=O)Nc2ccc(-c3ncn(-c4ccc(OC(F)(F)F)cc4)n3)cc2)c1] |
| J319 | 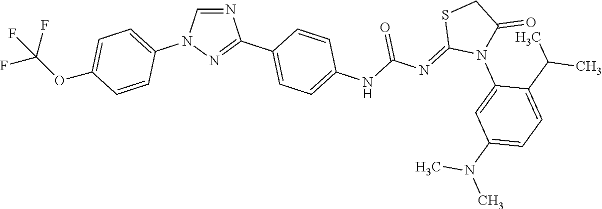 [SMILES: CC(C)c1ccc(N(C)C)cc1N1C(=O)CS/C1=N\C(=O)Nc1ccc(-c2ncn(-c3ccc(OC(F)(F)F)cc3)n2)cc1] |
| J320 | 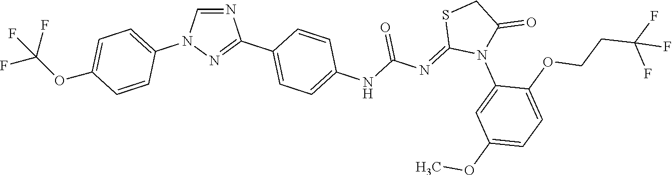 [SMILES: COc1ccc(OCCC(F)(F)F)c(N2C(=O)CS/C2=N\C(=O)Nc2ccc(-c3ncn(-c4ccc(OC(F)(F)F)cc4)n3)cc2)c1] |
| J321 | 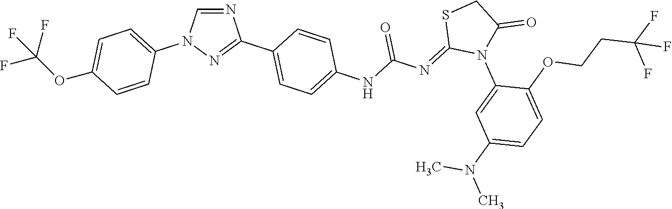 [SMILES: CN(C)c1ccc(OCCC(F)(F)F)c(N2C(=O)CS/C2=N\C(=O)Nc2ccc(-c3ncn(-c4ccc(OC(F)(F)F)cc4)n3)cc2)c1] |
| J322 | 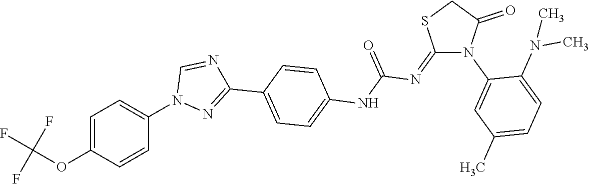 [SMILES: Cc1ccc(N(C)C)c(N2C(=O)CS/C2=N\C(=O)Nc2ccc(-c3ncn(-c4ccc(OC(F)(F)F)cc4)n3)cc2)c1] |
| J323 | 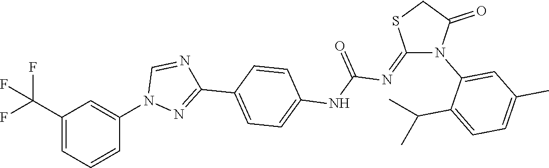 [SMILES: Cc1ccc(C(C)C)c(N2C(=O)CS/C2=N\C(=O)Nc2ccc(-c3ncn(-c4cccc(C(F)(F)F)c4)n3)cc2)c1] |
| J327 | 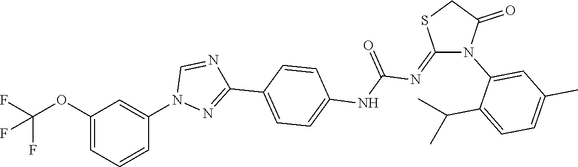 [SMILES: Cc1ccc(C(C)C)c(N2C(=O)CS/C2=N\C(=O)Nc2ccc(-c3ncn(-c4cccc(OC(F)(F)F)c4)n3)cc2)c1] |
| J331 | 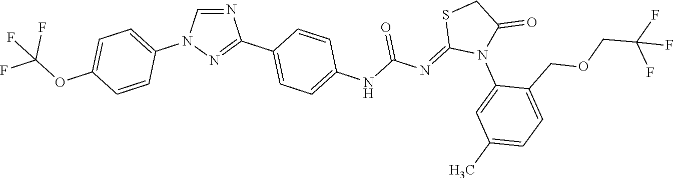 [SMILES: Cc1ccc(COCC(F)(F)F)c(N2C(=O)CS/C2=N\C(=O)Nc2ccc(-c3ncn(-c4ccc(OC(F)(F)F)cc4)n3)cc2)c1] |
| J332 | 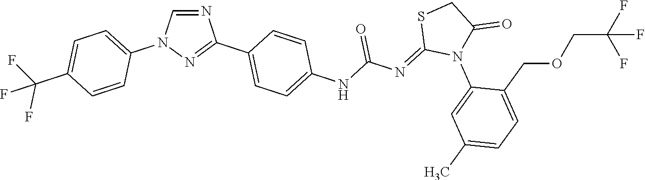 [SMILES: Cc1ccc(COCC(F)(F)F)c(N2C(=O)CS/C2=N\C(=O)Nc2ccc(-c3ncn(-c4ccc(C(F)(F)F)cc4)n3)cc2)c1] |
| J333 | 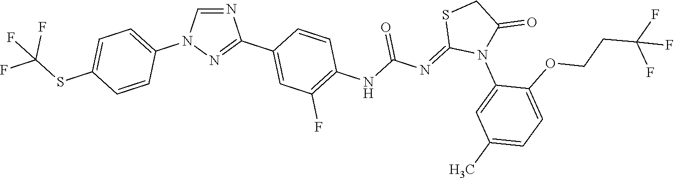 [SMILES: Cc1ccc(OCCC(F)(F)F)c(N2C(=O)CS/C2=N\C(=O)Nc2ccc(-c3ncn(-c4ccc(SC(F)(F)F)cc4)n3)cc2F)c1] |
| J334 | 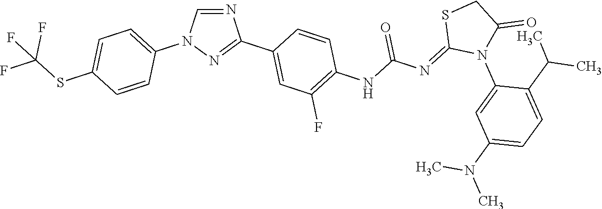 [SMILES: CC(C)c1ccc(N(C)C)cc1N1C(=O)CS/C1=N\C(=O)Nc1ccc(-c2ncn(-c3ccc(SC(F)(F)F)cc3)n2)cc1F] |
| J336 | 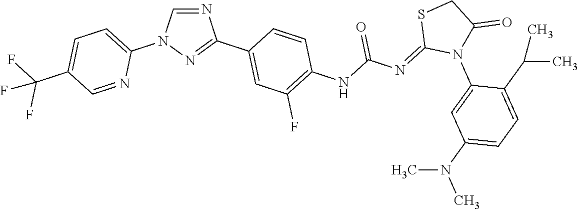 [SMILES: CC(C)c1ccc(N(C)C)cc1N1C(=O)CS/C1=N\C(=O)Nc1ccc(-c2ncn(-c3ccc(C(F)(F)F)cn3)n2)cc1F] |
| J337 | 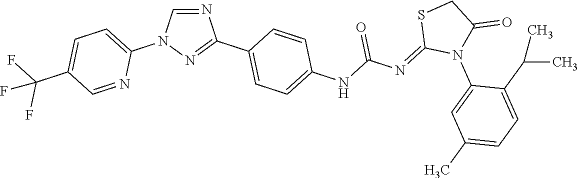 [SMILES: Cc1ccc(C(C)C)c(N2C(=O)CS/C2=N\C(=O)Nc2ccc(-c3ncn(-c4ccc(C(F)(F)F)cn4)n3)cc2)c1] |
| J338 | 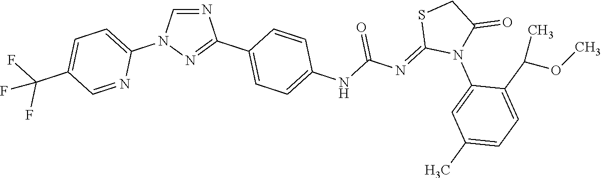 [SMILES: COC(C)c1ccc(C)cc1N1C(=O)CS/C1=N\C(=O)Nc1ccc(-c2ncn(-c3ccc(C(F)(F)F)cn3)n2)cc1] |
| J343 | 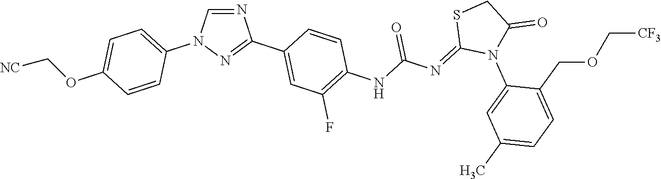 [SMILES: Cc1ccc(COCC(F)(F)F)c(N2C(=O)CS/C2=N\C(=O)Nc2ccc(-c3ncn(-c4ccc(OCC#N)cc4)n3)cc2F)c1] |
| J348 | 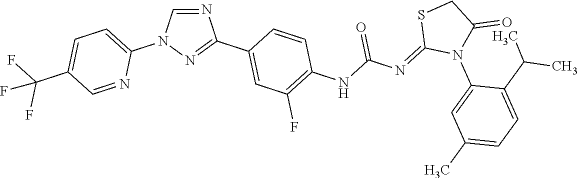 [SMILES: Cc1ccc(C(C)C)c(N2C(=O)CS/C2=N\C(=O)Nc2ccc(-c3ncn(-c4ccc(C(F)(F)F)cn4)n3)cc2F)c1] |
| J355 | 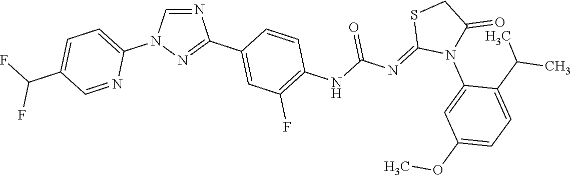 [SMILES: COc1ccc(C(C)C)c(N2C(=O)CS/C2=N\C(=O)Nc2ccc(-c3ncn(-c4ccc(C(F)F)cn4)n3)cc2F)c1] |
| J363 | 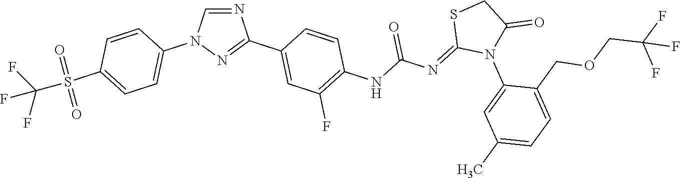 [SMILES: Cc1ccc(COCC(F)(F)F)c(N2C(=O)CS/C2=N\C(=O)Nc2ccc(-c3ncn(-c4ccc(S(=O)(=O)C(F)(F)F)cc4)n3)cc2F)c1] |
| J364 | 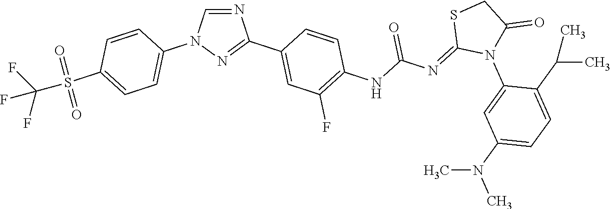 [SMILES: CC(C)c1ccc(N(C)C)cc1N1C(=O)CS/C1=N\C(=O)Nc1ccc(-c2ncn(-c3ccc(S(=O)(=O)C(F)(F)F)cc3)n2)cc1F] |
| J365 | 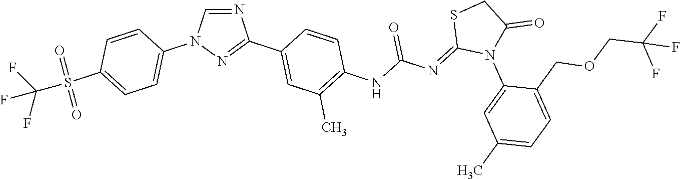 [SMILES: Cc1ccc(COCC(F)(F)F)c(N2C(=O)CS/C2=N\C(=O)Nc2ccc(-c3ncn(-c4ccc(S(=O)(=O)C(F)(F)F)cc4)n3)cc2C)c1] |
| J366 | 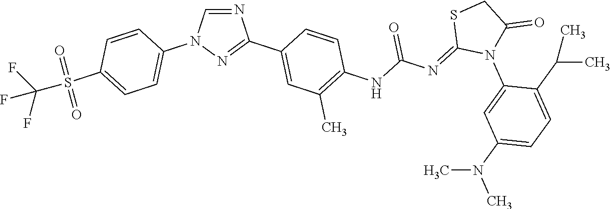 [SMILES: Cc1cc(-c2ncn(-c3ccc(S(=O)(=O)C(F)(F)F)cc3)n2)ccc1NC(=O)/N=C1\SCC(=O)N1c1cc(N(C)C)ccc1C(C)C] |
| J367 | 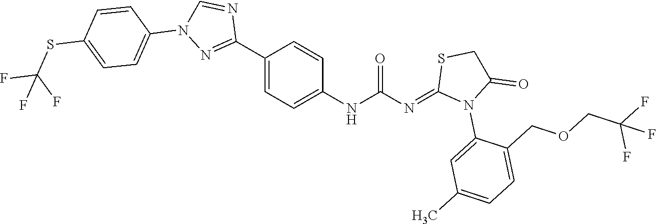 [SMILES: Cc1ccc(COCC(F)(F)F)c(N2C(=O)CS/C2=N\C(=O)Nc2ccc(-c3ncn(-c4ccc(SC(F)(F)F)cc4)n3)cc2)c1] |
| J368 | 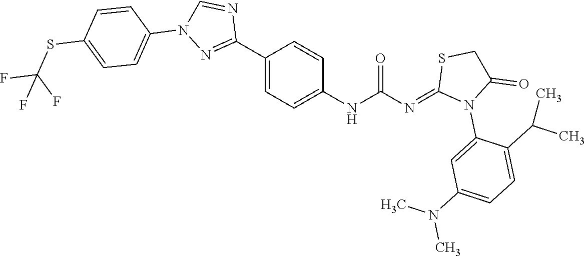 [SMILES: CC(C)c1ccc(N(C)C)cc1N1C(=O)CS/C1=N\C(=O)Nc1ccc(-c2ncn(-c3ccc(SC(F)(F)F)cc3)n2)cc1] |
| J373 | 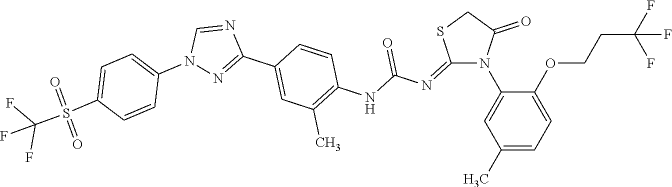 [SMILES: Cc1ccc(OCCC(F)(F)F)c(N2C(=O)CS/C2=N\C(=O)Nc2ccc(-c3ncn(-c4ccc(S(=O)(=O)C(F)(F)F)cc4)n3)cc2C)c1] |
| J379 | 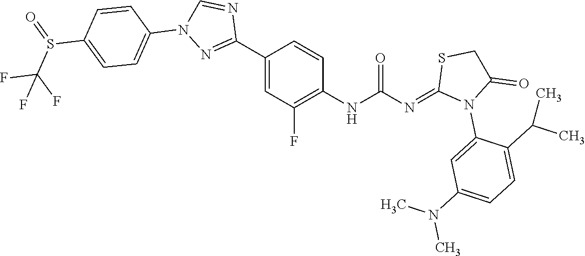 [SMILES: CC(C)c1ccc(N(C)C)cc1N1C(=O)CS/C1=N\C(=O)Nc1ccc(-c2ncn(-c3ccc(S(=O)C(F)(F)F)cc3)n2)cc1F] |
| J385 | 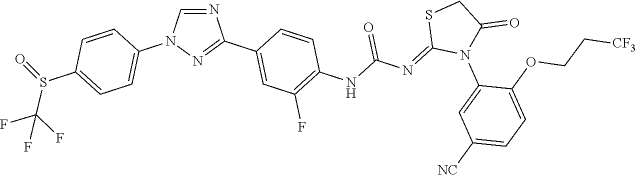 [SMILES: N#Cc1ccc(OCCC(F)(F)F)c(N2C(=O)CS/C2=N\C(=O)Nc2ccc(-c3ncn(-c4ccc(S(=O)C(F)(F)F)cc4)n3)cc2F)c1] |
| J391 | 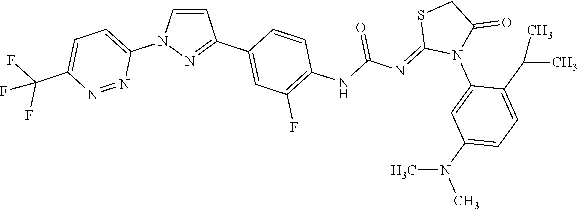 [SMILES: CC(C)c1ccc(N(C)C)cc1N1C(=O)CS/C1=N\C(=O)Nc1ccc(-c2ccn(-c3ccc(C(F)(F)F)nn3)n2)cc1F] |
| J401 | 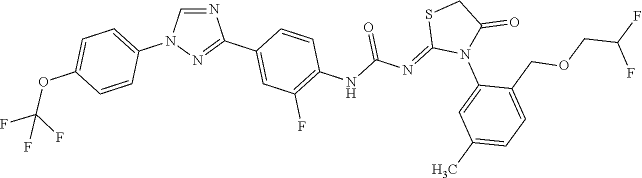 [SMILES: Cc1ccc(COCC(F)F)c(N2C(=O)CS/C2=N\C(=O)Nc2ccc(-c3ncn(-c4ccc(OC(F)(F)F)cc4)n3)cc2F)c1] |
| J405 | 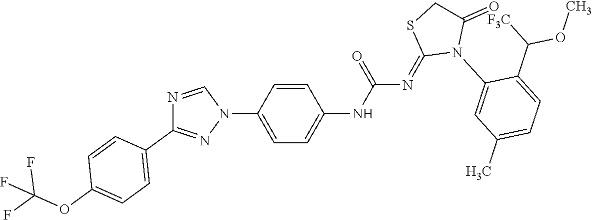 [SMILES: COC(c1ccc(C)cc1N1C(=O)CS/C1=N\C(=O)Nc1ccc(-n2cnc(-c3ccc(OC(F)(F)F)cc3)n2)cc1)C(F)(F)F] |
| J409 | 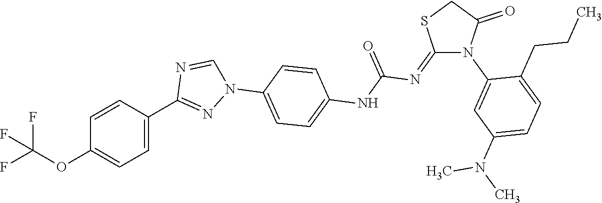 [SMILES: CCCc1ccc(N(C)C)cc1N1C(=O)CS/C1=N\C(=O)Nc1ccc(-n2cnc(-c3ccc(OC(F)(F)F)cc3)n2)cc1] |
| J421 | 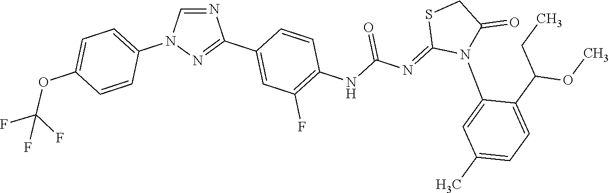 [SMILES: CCC(OC)c1ccc(C)cc1N1C(=O)CS/C1=N\C(=O)Nc1ccc(-c2ncn(-c3ccc(OC(F)(F)F)cc3)n2)cc1F] |
| J426 | 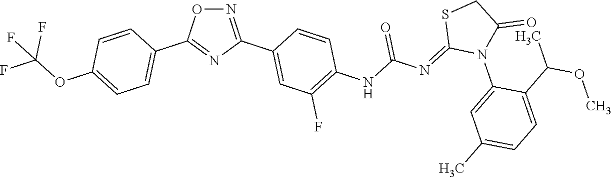 [SMILES: COC(C)c1ccc(C)cc1N1C(=O)CS/C1=N\C(=O)Nc1ccc(-c2noc(-c3ccc(OC(F)(F)F)cc3)n2)cc1F] |
| J435 | 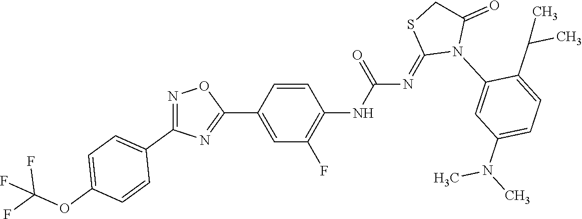 [SMILES: CC(C)c1ccc(N(C)C)cc1N1C(=O)CS/C1=N\C(=O)Nc1ccc(-c2nc(-c3ccc(OC(F)(F)F)cc3)no2)cc1F] |
| J446 | 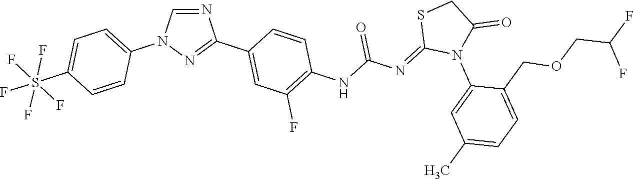 [SMILES: Cc1ccc(COCC(F)F)c(N2C(=O)CS/C2=N\C(=O)Nc2ccc(-c3ncn(-c4ccc(S(F)(F)(F)(F)F)cc4)n3)cc2F)c1] |
| J447 | 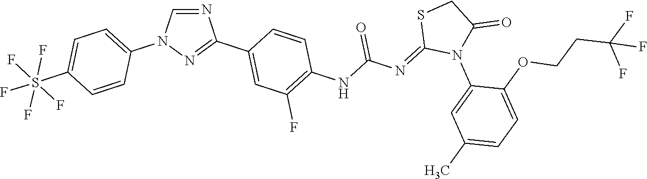 [SMILES: Cc1ccc(OCCC(F)(F)F)c(N2C(=O)CS/C2=N\C(=O)Nc2ccc(-c3ncn(-c4ccc(S(F)(F)(F)(F)F)cc4)n3)cc2F)c1] |
| J457 | 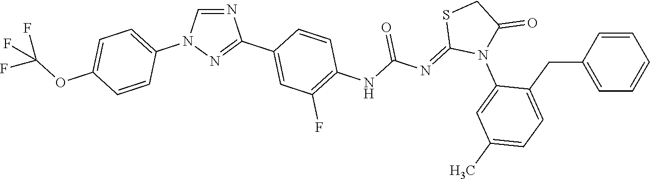 [SMILES: Cc1ccc(Cc2ccccc2)c(N2C(=O)CS/C2=N\C(=O)Nc2ccc(-c3ncn(-c4ccc(OC(F)(F)F)cc4)n3)cc2F)c1] |
| J465 | 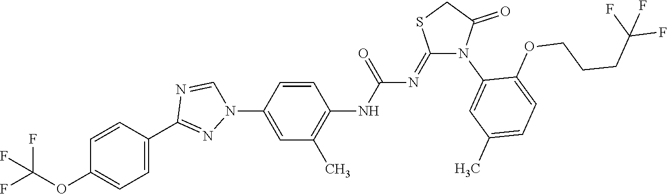 [SMILES: Cc1ccc(OCCCC(F)(F)F)c(N2C(=O)CS/C2=N\C(=O)Nc2ccc(-n3cnc(-c4ccc(OC(F)(F)F)cc4)n3)cc2C)c1] |
| J473 | 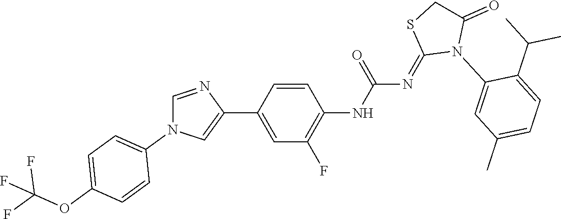 [SMILES: Cc1ccc(C(C)C)c(N2C(=O)CS/C2=N\C(=O)Nc2ccc(-c3cn(-c4ccc(OC(F)(F)F)cc4)cn3)cc2F)c1] |
| J474 | 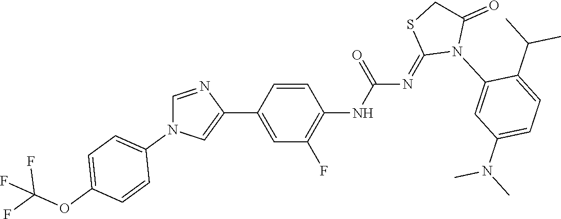 [SMILES: CC(C)c1ccc(N(C)C)cc1N1C(=O)CS/C1=N\C(=O)Nc1ccc(-c2cn(-c3ccc(OC(F)(F)F)cc3)cn2)cc1F] |
| J483 | 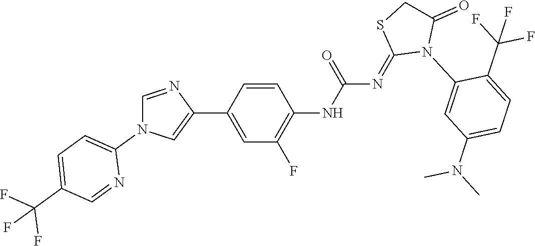 [SMILES: CN(C)c1ccc(C(F)(F)F)c(N2C(=O)CS/C2=N\C(=O)Nc2ccc(-c3cn(-c4ccc(C(F)(F)F)cn4)cn3)cc2F)c1] |
| J493 | 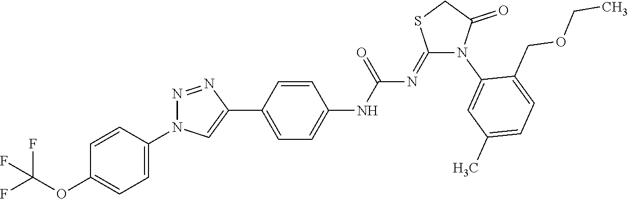 [SMILES: CCOCc1ccc(C)cc1N1C(=O)CS/C1=N\C(=O)Nc1ccc(-c2cn(-c3ccc(OC(F)(F)F)cc3)nn2)cc1] |
| J499 | 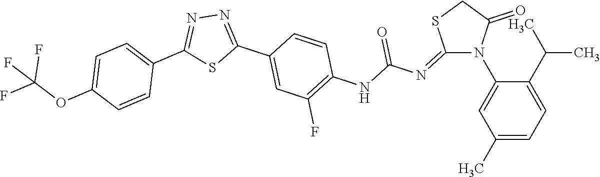 [SMILES: Cc1ccc(C(C)C)c(N2C(=O)CS/C2=N\C(=O)Nc2ccc(-c3nnc(-c4ccc(OC(F)(F)F)cc4)s3)cc2F)c1] |
| J507 | 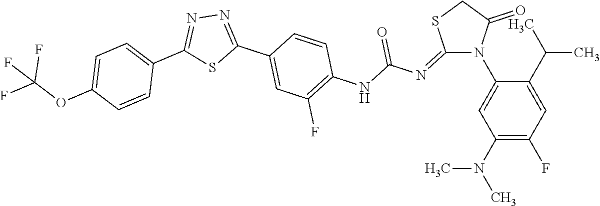 [SMILES: CC(C)c1cc(F)c(N(C)C)cc1N1C(=O)CS/C1=N\C(=O)Nc1ccc(-c2nnc(-c3ccc(OC(F)(F)F)cc3)s2)cc1F] |
| J510 | 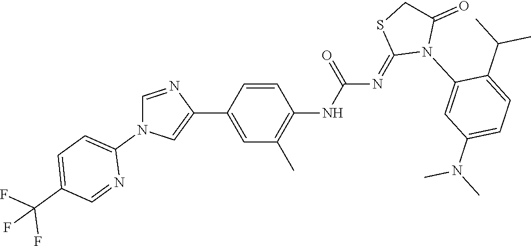 [SMILES: Cc1cc(-c2cn(-c3ccc(C(F)(F)F)cn3)cn2)ccc1NC(=O)/N=C1\SCC(=O)N1c1cc(N(C)C)ccc1C(C)C] |
| J514 | 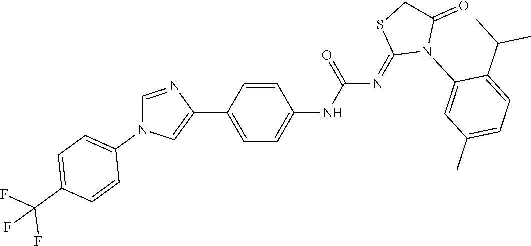 [SMILES: Cc1ccc(C(C)C)c(N2C(=O)CS/C2=N\C(=O)Nc2ccc(-c3cn(-c4ccc(C(F)(F)F)cc4)cn3)cc2)c1] |
| J515 | 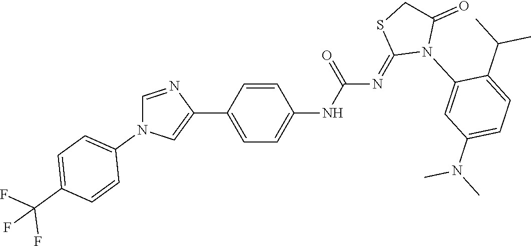 [SMILES: CC(C)c1ccc(N(C)C)cc1N1C(=O)CS/C1=N\C(=O)Nc1ccc(-c2cn(-c3ccc(C(F)(F)F)cc3)cn2)cc1] |
| J527 | 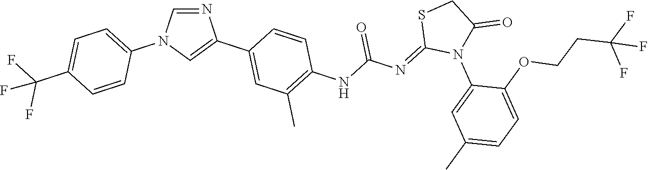 [SMILES: Cc1ccc(OCCC(F)(F)F)c(N2C(=O)CS/C2=N\C(=O)Nc2ccc(-c3cn(-c4ccc(C(F)(F)F)cc4)cn3)cc2C)c1] |
| J529 | 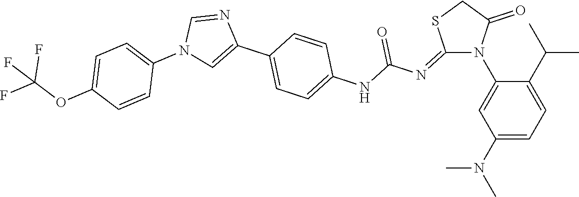 [SMILES: CC(C)c1ccc(N(C)C)cc1N1C(=O)CS/C1=N\C(=O)Nc1ccc(-c2cn(-c3ccc(OC(F)(F)F)cc3)cn2)cc1] |
| J541 | 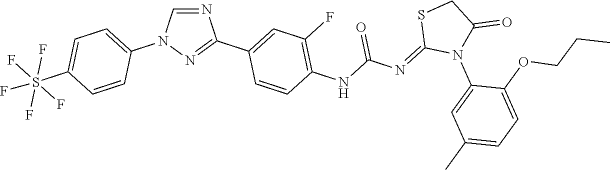 [SMILES: CCCOc1ccc(C)cc1N1C(=O)CS/C1=N\C(=O)Nc1ccc(-c2ncn(-c3ccc(S(F)(F)(F)(F)F)cc3)n2)cc1F] |
| J552 | 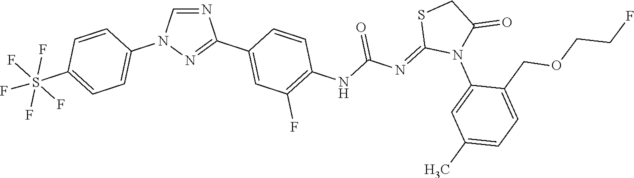 [SMILES: Cc1ccc(COCCF)c(N2C(=O)CS/C2=N\C(=O)Nc2ccc(-c3ncn(-c4ccc(S(F)(F)(F)(F)F)cc4)n3)cc2F)c1] |
| J555 | 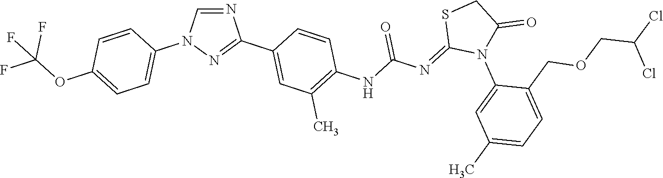 [SMILES: Cc1ccc(COCC(Cl)Cl)c(N2C(=O)CS/C2=N\C(=O)Nc2ccc(-c3ncn(-c4ccc(OC(F)(F)F)cc4)n3)cc2C)c1] |
| J563 | 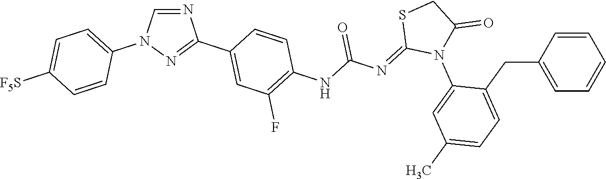 |
| J568 | 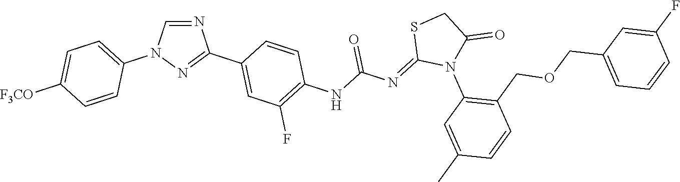 [SMILES: Cc1ccc(COCc2cccc(F)c2)c(N2C(=O)CS/C2=N\C(=O)Nc2ccc(-c3ncn(-c4ccc(OC(F)(F)F)cc4)n3)cc2F)c1] |
| J571 | 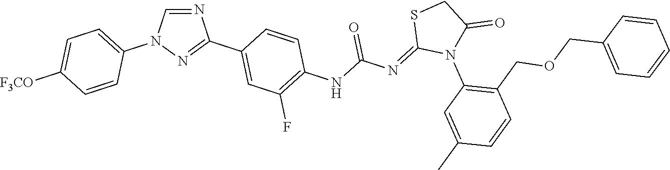 [SMILES: Cc1ccc(COCc2ccccc2)c(N2C(=O)CS/C2=N\C(=O)Nc2ccc(-c3ncn(-c4ccc(OC(F)(F)F)cc4)n3)cc2F)c1] |
| J582 | 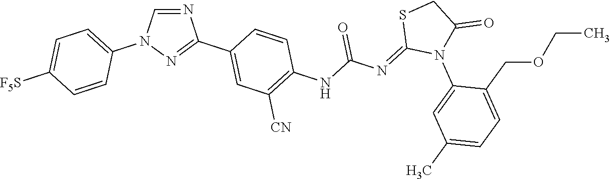 |
| J586 | 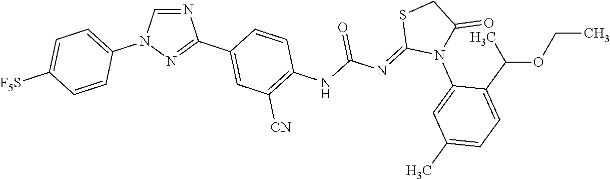 |
| J599 | 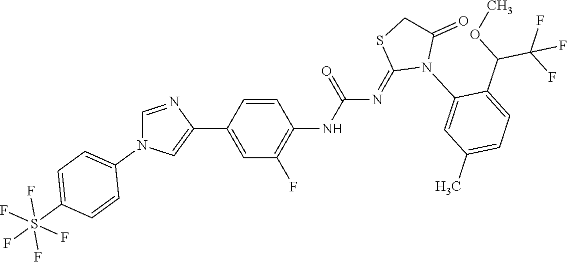 [SMILES: COC(c1ccc(C)cc1N1C(=O)CS/C1=N\C(=O)Nc1ccc(-c2cn(-c3ccc(S(F)(F)(F)(F)F)cc3)cn2)cc1F)C(F)(F)F] |
| J603 | 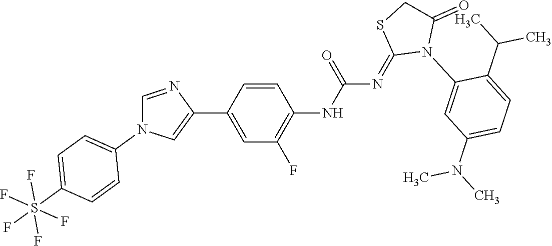 [SMILES: CC(C)c1ccc(N(C)C)cc1N1C(=O)CS/C1=N\C(=O)Nc1ccc(-c2cn(-c3ccc(S(F)(F)(F)(F)F)cc3)cn2)cc1F] |
| J664 | 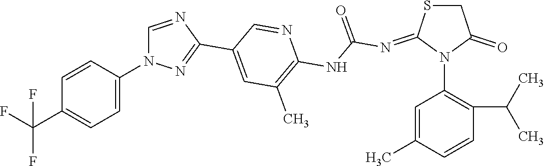 [SMILES: Cc1ccc(C(C)C)c(N2C(=O)CS/C2=N\C(=O)Nc2ncc(-c3ncn(-c4ccc(C(F)(F)F)cc4)n3)cc2C)c1] |
| J735 | 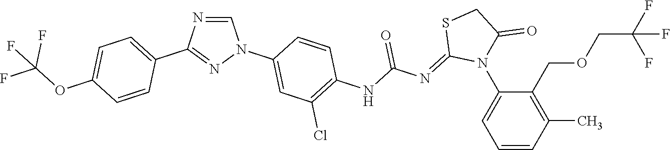 [SMILES: Cc1cccc(N2C(=O)CS/C2=N\C(=O)Nc2ccc(-n3cnc(-c4ccc(OC(F)(F)F)cc4)n3)cc2Cl)c1COCC(F)(F)F] |
| J737 | 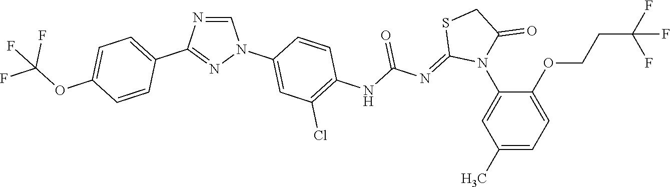 [SMILES: Cc1ccc(OCCC(F)(F)F)c(N2C(=O)CS/C2=N\C(=O)Nc2ccc(-n3cnc(-c4ccc(OC(F)(F)F)cc4)n3)cc2Cl)c1] |
| J751 | 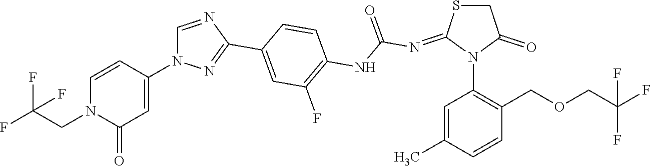 [SMILES: Cc1ccc(COCC(F)(F)F)c(N2C(=O)CS/C2=N\C(=O)Nc2ccc(-c3ncn(-c4ccn(CC(F)(F)F)c(=O)c4)n3)cc2F)c1] |
| J772 | 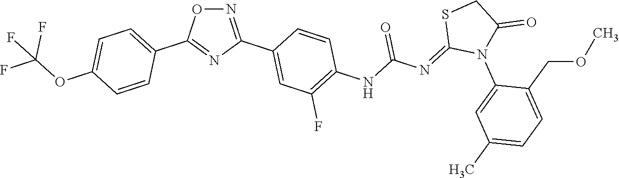 [SMILES: COCc1ccc(C)cc1N1C(=O)CS/C1=N\C(=O)Nc1ccc(-c2noc(-c3ccc(OC(F)(F)F)cc3)n2)cc1F] |
| J783 | 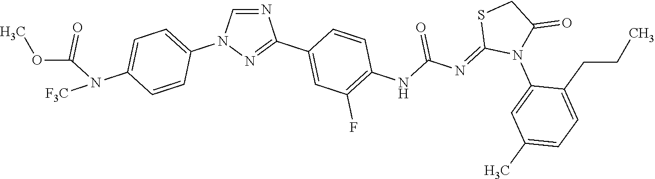 [SMILES: CCCc1ccc(C)cc1N1C(=O)CS/C1=N\C(=O)Nc1ccc(-c2ncn(-c3ccc(N(C(=O)OC)C(F)(F)F)cc3)n2)cc1F] |
| J790 | 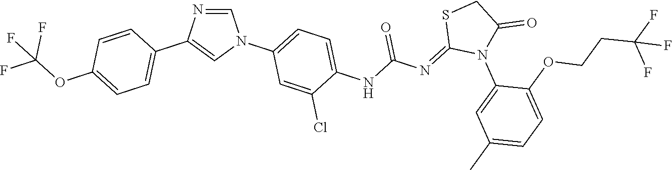 [SMILES: Cc1ccc(OCCC(F)(F)F)c(N2C(=O)CS/C2=N\C(=O)Nc2ccc(-n3cnc(-c4ccc(OC(F)(F)F)cc4)c3)cc2Cl)c1] |
| J793 | 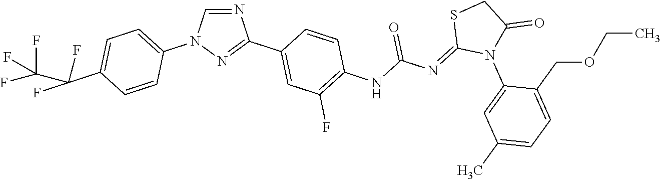 [SMILES: CCOCc1ccc(C)cc1N1C(=O)CS/C1=N\C(=O)Nc1ccc(-c2ncn(-c3ccc(C(F)(F)C(F)(F)F)cc3)n2)cc1F] |
| J794 | 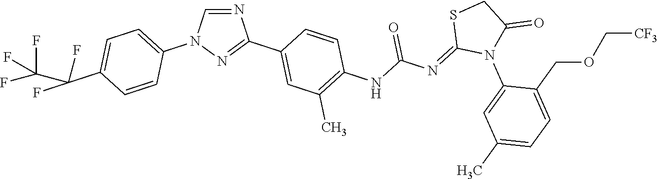 [SMILES: Cc1ccc(COCC(F)(F)F)c(N2C(=O)CS/C2=N\C(=O)Nc2ccc(-c3ncn(-c4ccc(C(F)(F)C(F)(F)F)cc4)n3)cc2C)c1] |

In one embodiment, also provided is a process comprising applying the compound provided herein to a locus to control a pest, in an amount sufficient to control such pest.

In a further embodiment, wherein said pest is beet armyworm (BAW), cabbage looper (CL), or yellow fever mosquito (YFM).

In another embodiment, the compound provided herein has at least one ²H or at least one ¹⁴C.

In another embodiment, also provided is a composition comprising the compound provided herein and at least one other compound having insecticidal, herbicidal, acaricidal, nematicidal, or fungicidal activity.

In another embodiment, also provided is a composition comprising the compound provided herein and a seed.

In another embodiment, also provided is a process comprising applying the compound provided herein to a genetically modified plant, or genetically-modified seed, which has been genetically modified to express one or more specialized traits.

In another embodiment, also provided is a process comprising: orally administering; or topically applying; the compound provided herein, to a non-human animal, to control endoparasites and/or ectoparasites.

Many of the molecules of Formula One may be depicted in two or more tautomeric forms such as when R¹, R², or R⁴, is H (see for example, “Scheme TAU” below). For the sake of simplifying the schemes, all molecules have been depicted as existing as a single tautomer. Any and all alternative tautomers are included within the scope of this Formula One, and no inference should be made as to whether the molecule exists as the tautomeric form in which it is drawn.

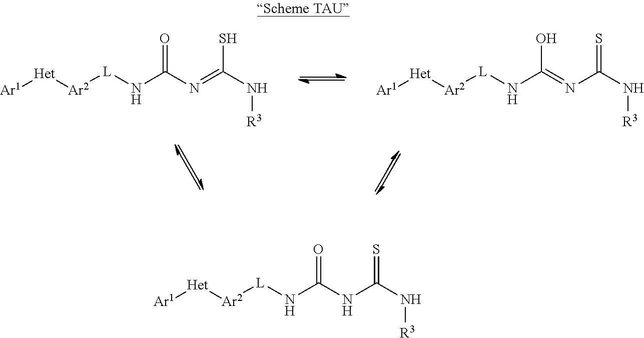

The molecules of Formula One will generally have a molecular mass of about 400 Daltons to about 1200 Daltons. However, it is generally preferred if the molecular mass is from about 300 Daltons to about 1000 Daltons, and it is even more generally preferred if the molecular mass is from about 400 Daltons to about 750 Daltons.

### Preparation of Thiobiurets

Thiobiurets disclosed herein are prepared from the corresponding isocyanate, Ar¹-Het-Ar²-L-NCO (1-2). Usually, these isocyanates are not isolated, but are instead generated in situ from a suitable precursor and used directly in the preparation of a thiobiuret. One such suitable precursor is an amine (1-1) which can be converted into an isocyanate by using one of several common reagents such as phosgene, diphosgene, triphosgene, or carbonyldiimidazole (Scheme 1, step a), in a mixed solvent system such as dichloromethane and water or diethyl ether and water, in the presence of a base such as sodium bicarbonate or triethylamine, at temperatures from about −10° C. to about 50° C.

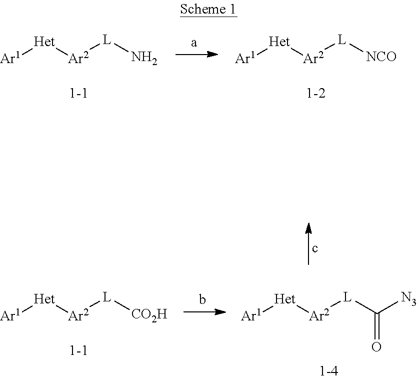

Alternatively, the isocyanates may be generated via a Curtius rearrangement of an acyl azide, Ar¹-Het-Ar²-L-C(═O)N₃ (1-4), which is, in turn, prepared from the corresponding carboxylic acid precursor, Ar¹—Het-Ar²-L-CO₂H (1-3). Formation of an acyl azide (Scheme 1, step b) occurs either by treatment of the acid with ethyl chloroformate and sodium azide in the presence of an amine base such as triethylamine, or with diphenylphosphoryl azide in the presence of an amine base such as triethylamine. The acyl azide is then made to undergo a Curtius rearrangement (which may need to be thermally induced), leading to the corresponding isocyanate (1-2). Depending on the nature of the particular acyl azide, this rearrangement may occur spontaneously at ambient temperature, or it may require heating from about 40° C. to about 100° C. in a suitable solvent, such as toluene, or acetonitrile, or an ethereal solvent such as dioxane or tetrahydrofuran. Azides of arylacetic acids are known, though frequently, due to their reactivity, they are not isolated as pure solids. Accordingly, the acyl azide intermediate is not always fully characterized, but may simply be heated directly without characterization, to generate the isocyanate.

An isocyanate, Ar¹-Het-Ar²-L-NCO (1-2), can be treated directly with an N-aryl thiourea (2-1) in the presence of about 0.1 to about 2 equivalents of an inorganic base such as cesium carbonate or sodium hydride, resulting in the formation of a thiobiuret (2-2, Scheme 2). The reaction can be performed at temperatures from about 0° C. to about 100° C., preferably from about 20° C. to about 80° C., in an aprotic solvent or solvent mixture chosen from acetonitrile, acetone, toluene, tetrahydrofuran, 1,2-dichloroethane, dichloromethane, or mixtures thereof, but use of acetonitrile is preferred.

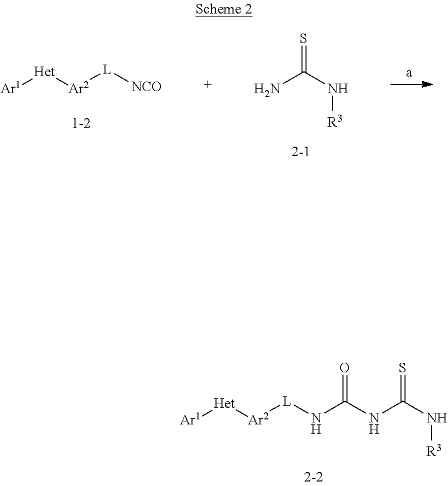

Thiobiurets (2-2) generated in situ can be converted directly without purification into a variety of cyclized analogs (Scheme 3), or they can be isolated from the reaction medium prior to cyclization. Cyclization can be achieved by treatment with an α-halo ester such as methyl bromoacetate to form 2-imino 1,3-thiazolin-4-ones (3-1, step a) unsubstituted or mono- or di-substituted with R¹; vicinal dihalides such as 1-bromo-2-chloroethane or 1,2-dichloroethane, to form 2-imino-1,3-thiazolines (3-2, step b) unsubstituted or mono-substituted with R⁵ or R⁶; α-halo ketones such as chloroacetone to form 2-imino-1,3-thiazoles (3-3, step c) unsubstituted with R⁵ or R⁶; or 1,3-dihalopropanes such as 1-bromo-3-chloro-propane to form 2-imino-1,3-thiazinanes (3-4, step d) unsubstituted or mono-substituted with R⁵ or R⁶ or unsubstituted or mono- or di-substituted with R⁷. With step a, use of sodium acetate in a protic solvent such as ethanol or methanol, at temperatures ranging from about 20° C. to about 70° C. is preferred. With step b, use of an inorganic base such as potassium carbonate in a solvent such as acetonitrile or (preferably) 2-butanone, at a temperature between about 0° C. and about 80° C., is preferred.

An alternative method for preparing analogs having the general structure 3-1′ (Scheme 3) is described in Scheme 3a. Intermediate 4-imino-3-arylthiazolidinone-2-one (3-1a, step a) is reacted directly with an isocyanate (1-2), in the presence of about 0.1 to about 2 equivalents of an inorganic base such as cesium carbonate or sodium hydride to form cyclized thiobiuret (3-1′). The reaction can be performed at temperatures from about 0° C. to about 100° C., preferably from about 20° C. to about 80° C., in an aprotic solvent or solvent mixture chosen from acetonitrile, acetone, toluene, tetrahydrofuran, 1,2-dichloroethane, dichloromethane, or mixtures thereof, but use of acetonitrile is preferred.

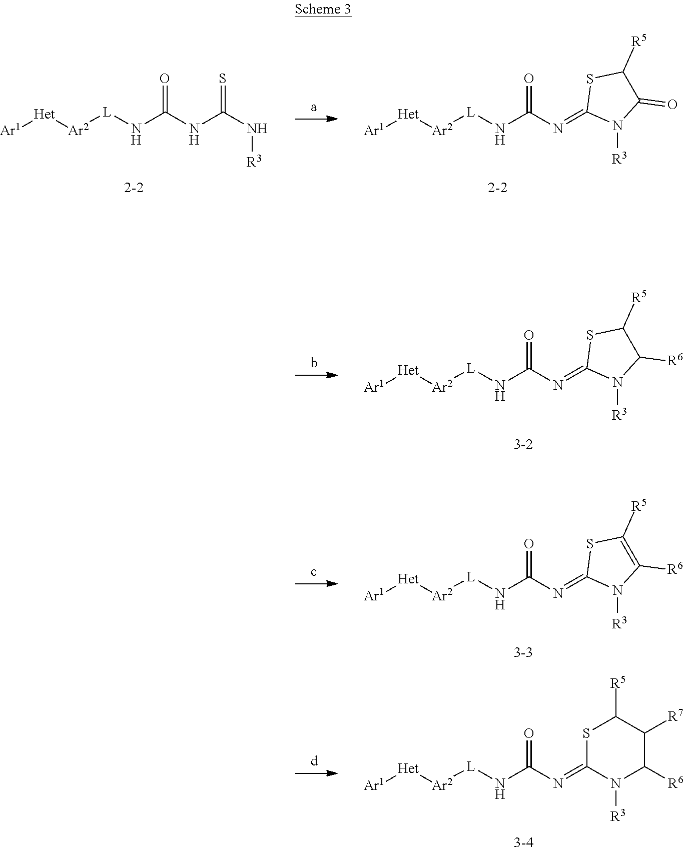

Alternatively, the 4-imino-3-arylthiazolidinone-2-one (3-1a) may be reacted with 4-nitrophenyl chloroformate (step b), forming a 4-nitrophenyl carbamate intermediate (3-2a). This reaction is conducted with equimolar quantities of the imine and the chloroformate, in a polar aprotic solvent such as tetrahydrofuran or dioxane, and in the presence of from about 0.1 to about 2 equivalents of an inorganic base such as cesium carbonate or potassium carbonate, preferably at room temperature. The intermediate (3-2a) may be isolated by filtration from inorganic salts and evaporation of solvent, or it can be used directly in step c. In step c, treatment of 3-2a with a primary or secondary alkyl amine Ar₁-Het-Ar₂-L-NHR¹, wherein R¹ is H or alkyl, respectively, may generate cyclized thiobiuret (3-1′). Step c may also be conducted in the presence of an inorganic base such as cesium carbonate or potassium carbonate, from about 0.1 to about 2 equivalents, preferably about 1 to about 1.2 equivalents; it is also most conveniently undertaken at room temperature, although it may be undertaken at temperatures from about 0° C. to about 100° C.

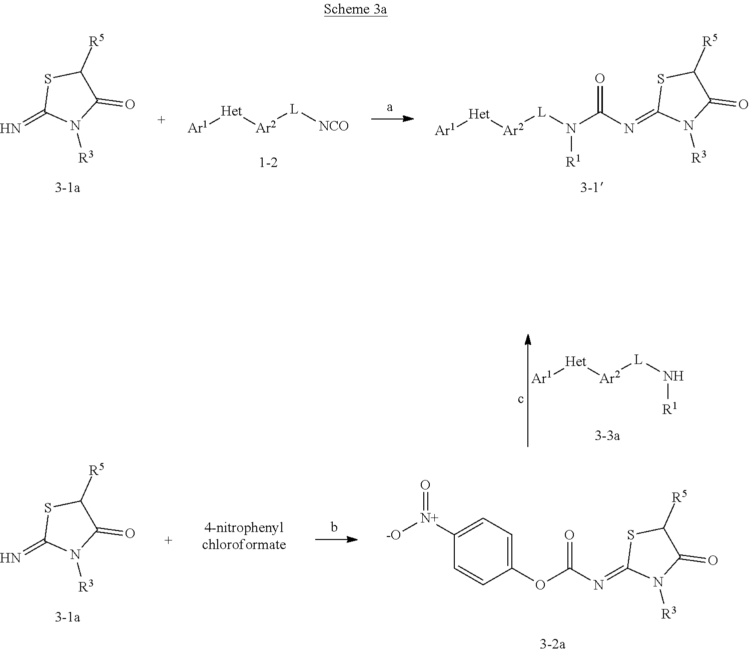

Alternatively, treatment of a primary or secondary alkyl amine 3-3a Ar₁-Het-Ar₂-L-NHR¹, wherein R¹ is H or alkyl, respectively, with an activating agent such bis(2,5-dioxopyrrolidin-1-yl) carbonate in the presence of an organic base such as pyridine, preferably from about 1 to about 1.2 equivalents, triphosgene in the presence of an organic base such as N,N-diisopropylethylamine, or 4-nitrophenyl carbonochloridate in an aprotic solvent such as dichloromethane may generate in situ an activated amine (not shown), which is reacted with 4-imino-3-arylthiazolidinone-2-one (3-1a) with or without an organic base such as N,N-diisopropylethylamine to afford the cyclized thiobiuret (3-1′) as in step a, Scheme 3a′.

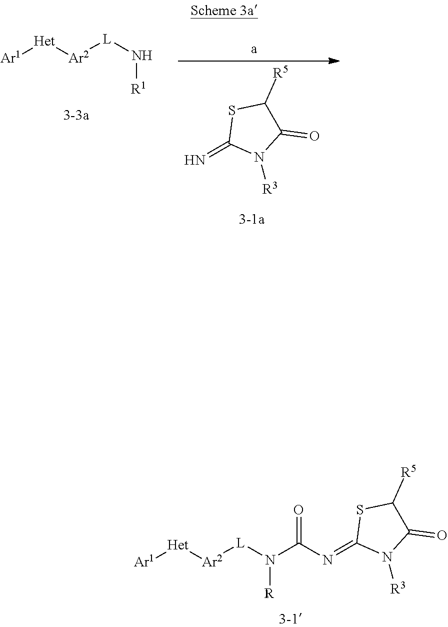

Thiobiurets (2-2) can also be converted into novel S-alkylated analogs as described in Scheme 3b. For example, reaction of a thiobiuret 2-2 with an alkyl iodide (step a), in a protic solvent such as ethanol, and in the presence of a base such as sodium acetate, at temperatures from about 0° C. to about 60° C., results in formation of an S—R² substituted product (3-1b). A variation of the reaction conditions described in Scheme 3, step c, employs careful control of reaction conditions to ensure that the temperature does not exceed 20° C. Under these conditions, 4-hydroxy-2-iminothiazolidines (3-2b, step b) may be isolated.

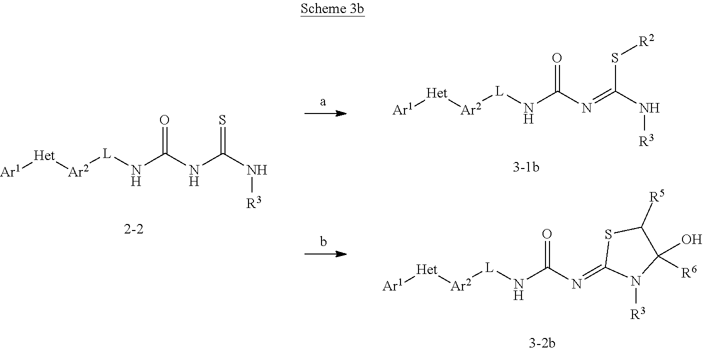

Analogs of Formula One wherein R² and R⁴ are cyclized to form a 2-(R⁵)-4-(R³)-5-imino-1,2,4-thiadiazolidin-3-one (3-4c) may be constructed as described in Scheme 3c. Following the work described by Kaugers, et al (J. Org. Chem. 1992, 57, 1671), an N-arylamino 1,2,3,4-thiatriazole (3-1c), prepared in one step from the corresponding N³-aryl thiosemicarbazone by oxidation with sodium nitrite, is treated with an alkyl isocyanate to form 3-2c. Treatment of 3-2c with a base such as sodium methoxide in methanol at room temperature (step b) results in cleavage of the urea bond and formation of a 2-(R⁵)-4-(R³)-5-imino-1,2,4-thiadiazolidin-3-one (3-3c). This imine may then be treated with an isocyanate under conditions equivalent to those described in Scheme 3a, step a, to form 3-4c.

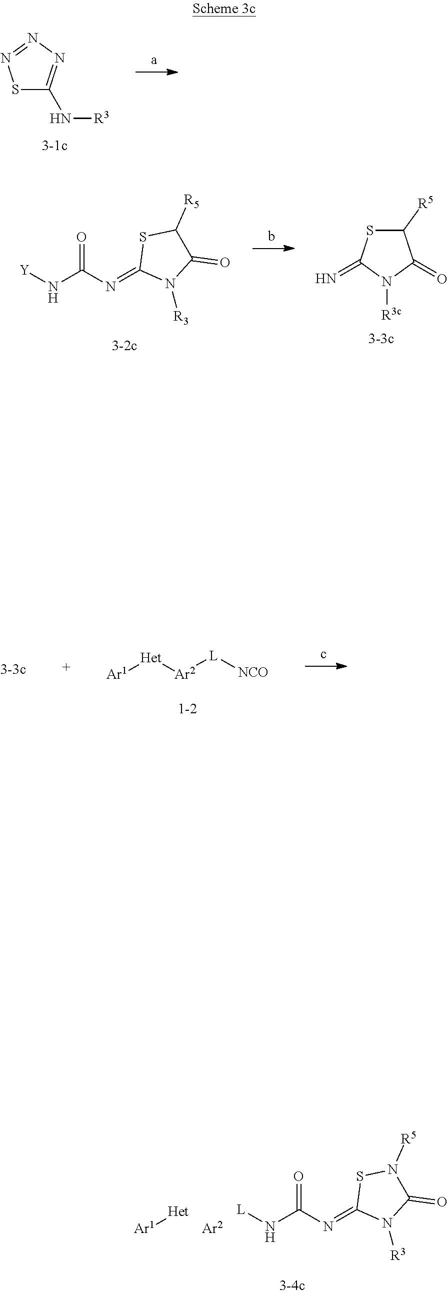

### Preparation of Triaryl-Intermediates

Molecules of Formula One can be prepared by making a triaryl intermediate, Ar¹-Het-Ar², and then linking it to an appropriate intermediate to form a desired compound. A wide variety of triaryl intermediates can be used to prepare molecules of Formula One, provided that such triaryl intermediates contain a suitable functional group on Ar² to which the rest of the desired functional group can be attached. Suitable functional groups include an amino, amino via nitro, isocyanate, carboxyl, or a halogen (preferably bromo or iodo). These triaryl intermediates can be prepared by methods previously described in the chemical literature, including Crouse, et al., WO2009102736 (the entire disclosure of which is hereby incorporated by reference).

The triaryl aldehydes used as precursors in preparation of the molecules of Formula One can be prepared according to procedures described in Crouse, et al., US 2012/0202688 A1. Some of the procedures described above require use of halo-aryl intermediates, Ar¹-Het-Ph-Br, which are novel intermediates. These may be prepared as described in Scheme 4. 3-(4-Bromophenyl)-1,2,4-triazole (4-2, step a) is prepared in two steps from 4-bromobenzamide (4-1) under conditions described previously (Crouse, et al., WO2009102736). This triazole can then be coupled to an aryl halide (R=C₁-C₆ haloalkoxy) such as 4-trifluoromethoxyphenyl bromobenzene, in the presence of cesium carbonate or potassium phosphate, in a polar aprotic solvent such as dimethylformamide. This reaction is catalyzed by a copper salt such as copper(I) iodide and a chelator such as 8-hydroxyquinoline, both present in about 0.05 to about 0.25 equivalents, at a temperature ranging between about 80° C. and about 140° C., to form the 1-aryl-3-(4-bromophenyl) triazole (4-4, step b).

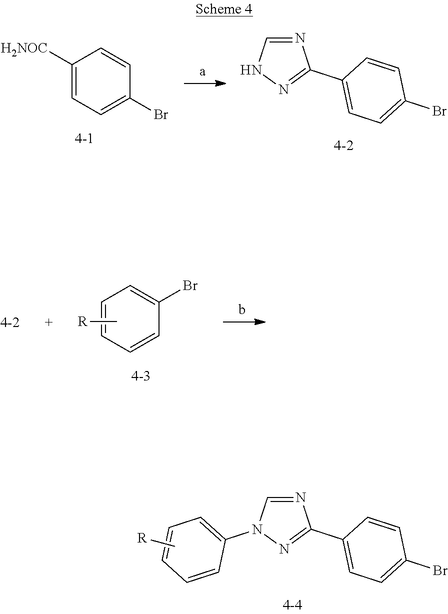

Other triaryl intermediates, Ar¹-Het-Ar², wherein Het is defined as one of Het-C through Het-L can be prepared be as in Schemes 4A through 4H

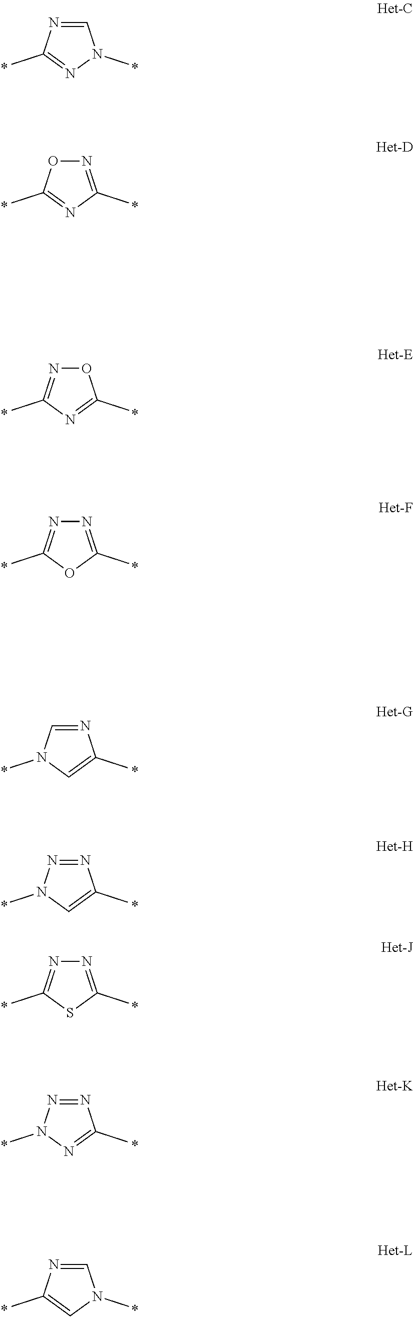

Triaryl intermediates, Ar¹-Het-Ar², wherein Het is Het-D (1,2,4-oxadiazole) can be prepared according to Scheme 4A in a four-step process. The first two steps (a and b) are adapted from Baykov, S. et al. Tetrahedron Lett. 2016, 57, 2898-2900, wherein a benzonitrile, such as 3-fluoro-4-nitrobenzonitrile, is transformed into the hydroxybenimidamide, 4a-1, by treatment with hydroxylamine in ethanol under thermal conditions. Reaction of 4a-1 with a benzoyl chloride in the presence of a base such as N,N-diisopropylethylamine and in a solvent such as dichloromethane provides the benzoyloxybenzimidamide 4a-2. The triaryl nitro intermediate (not shown, step c) can be achieved by treatment of 4a-2 with tetrabutylammonium hydroxide (TBAH) in tetrahydrofuran (THF) (adapted from Otaka, H.; Ikeda, J.; Tanaka, D.; Tobe, M. Tetrahedron Lett. 2014, 55, 979-981). The nitro group can be reduced using standard reduction conditions involving iron powder, ammonium chloride in an ethanol-water solvent mixture at 80° C. to provide triaryl intermediate 4a-3, as in step d.

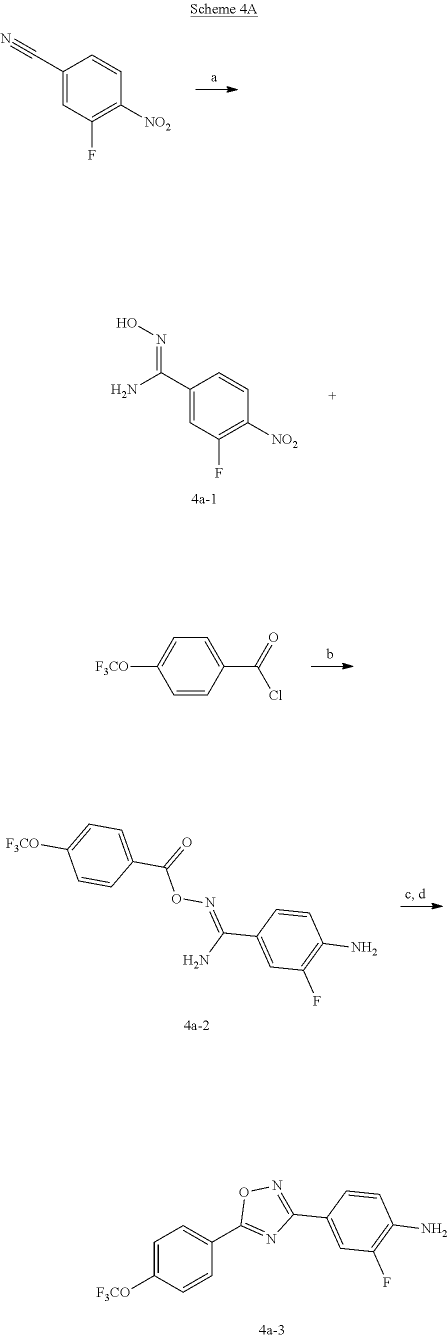 [SMILES: N#Cc1ccc([N+](=O)[O-])c(F)c1 ; N/C(=N\O)c1ccc([N+](=O)[O-])c(F)c1 ; O=C(Cl)c1ccc(OC(F)(F)F)cc1 ; N/C(=N\OC(=O)c1ccc(OC(F)(F)F)cc1)c1ccc(N)c(F)c1 ; Nc1ccc(-c2noc(-c3ccc(OC(F)(F)F)cc3)n2)cc1F]

Triaryl intermediates, Ar¹-Het-Ar², wherein Het is Het-E (reversed 1,2,4-oxadiazole) can be prepared according to Scheme 4B in a three-step process. In step a, a benzonitrile, such as 4-(trifluoromethoxy)benzonitrile, is transformed into the hydroxybenimidamide, 4b-1, by treatment with hydroxylamine in ethanol under thermal conditions. Reaction of 4b-1 with a benzoic acid in the presence of an activating agent such as 2,4,6-tripropyl-1,3,5,2,4,6-trioxatriphosphorinane-2,4,6-trioxide (T3P), in the presence of a base such as pyridine in a solvent such as EtOAc at elevated temperature (60° C.) provides triaryl nitro intermediate 4b-2. This T3P-mediated cyclization is adapted from Augustine, J. K. et al. Tetrahedron 2009, 65, 9989-9996. The nitro group on 4b-2 can be reduced using standard reduction conditions involving iron powder and ammonium chloride in an ethanol-water solvent mixture at 80° C. to provide triaryl intermediate 4b-3, as in step c.

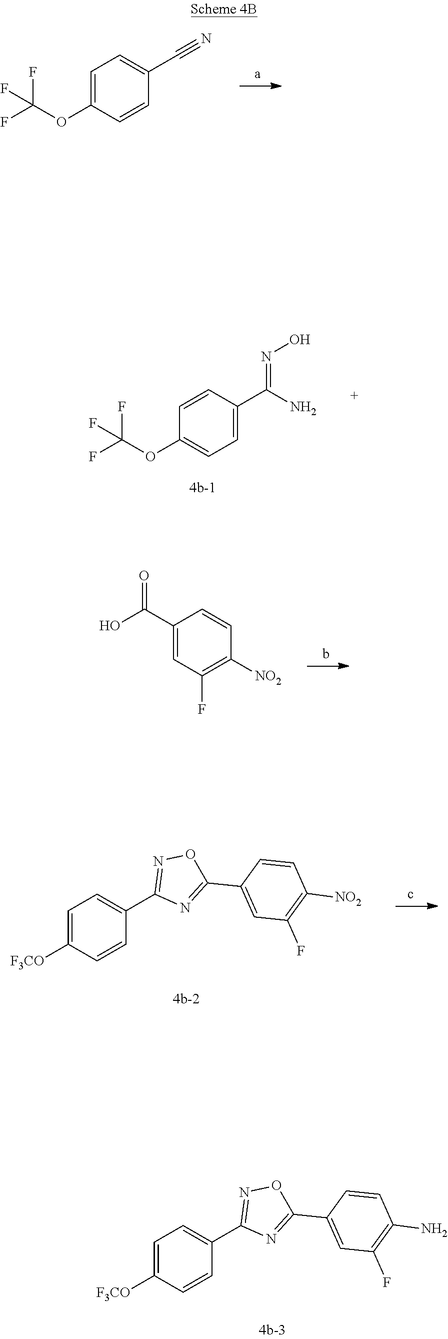 [SMILES: N#Cc1ccc(OC(F)(F)F)cc1 ; N/C(=N\O)c1ccc(OC(F)(F)F)cc1 ; O=C(O)c1ccc([N+](=O)[O-])c(F)c1 ; O=[N+]([O-])c1ccc(-c2nc(-c3ccc(OC(F)(F)F)cc3)no2)cc1F ; Nc1ccc(-c2nc(-c3ccc(OC(F)(F)F)cc3)no2)cc1F]

Triaryl intermediates, Ar¹-Het-Ar², wherein Het is Het-F (1,3,4-oxadiazole) can be prepared according to Scheme 4C in a two-step process. In step a, a benzohydrazide, such as 4-(trifluoromethoxy)benzohydrazide, is transformed into the triaryl nitro intermediate 4c-1, by treatment with an activating agent such as T3P, in the presence of a base such as pyridine and in a solvent such as EtOAc at elevated temperature (60° C.). This T3P-mediated cyclization is adapted from Augustine, J. K. et al. Tetrahedron 2009, 65, 9989-9996. The nitro group on 4c-1 can be reduced using standard reduction conditions involving iron powder and ammonium chloride in an ethanol-water solvent mixture at 80° C. to provide triaryl intermediate 4b-3, as in step b.

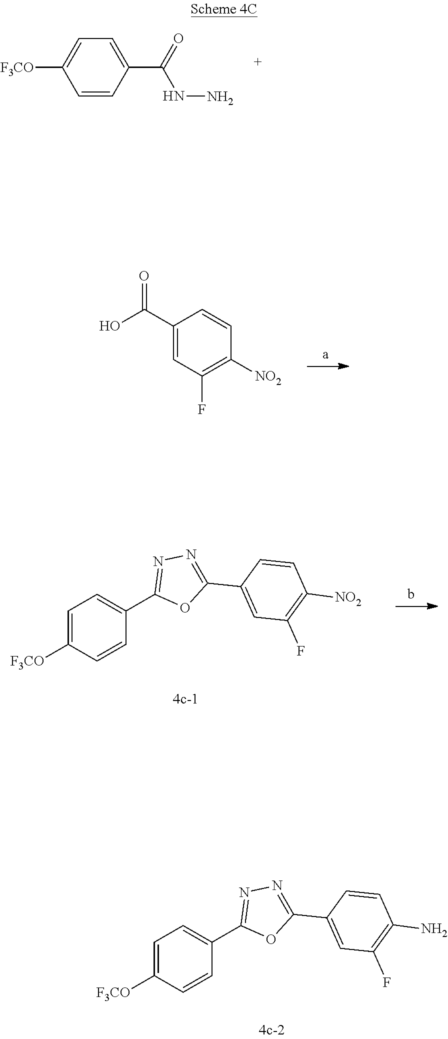 [SMILES: NNC(=O)c1ccc(OC(F)(F)F)cc1 ; O=C(O)c1ccc([N+](=O)[O-])c(F)c1 ; O=[N+]([O-])c1ccc(-c2nnc(-c3ccc(OC(F)(F)F)cc3)o2)cc1F ; Nc1ccc(-c2nnc(-c3ccc(OC(F)(F)F)cc3)o2)cc1F]

Triaryl intermediates, Ar¹-Het-Ar², wherein Het is Het-J (1,3,4-thiadiazole) can be prepared according to Scheme 4D in a three-step process. In step a, a benzohydrazide, such as 4-(trifluoromethoxy)benzohydrazide, is transformed into the benzoyl benzohydrazide 4d-1 by treatment with an activating agent such as T3P, in the presence of a base such as pyridine and in a solvent such as EtOAc at room temperature. Reaction of 4d-1 with Lawesson's reagent and T3P in the presence of a base such as pyridine and in a solvent such as EtOAc at elevated temperature (60° C.) provides the triaryl nitro intermediate (not shown, step b). This T3P-mediated cyclization is adapted from Augustine, J. K. et al. Tetrahedron 2009, 65, 9989-9996. The nitro group can be reduced using standard reduction conditions involving iron powder and ammonium chloride in an ethanol-water solvent mixture at 80° C. to provide triaryl intermediate 4d-2, as in step c.

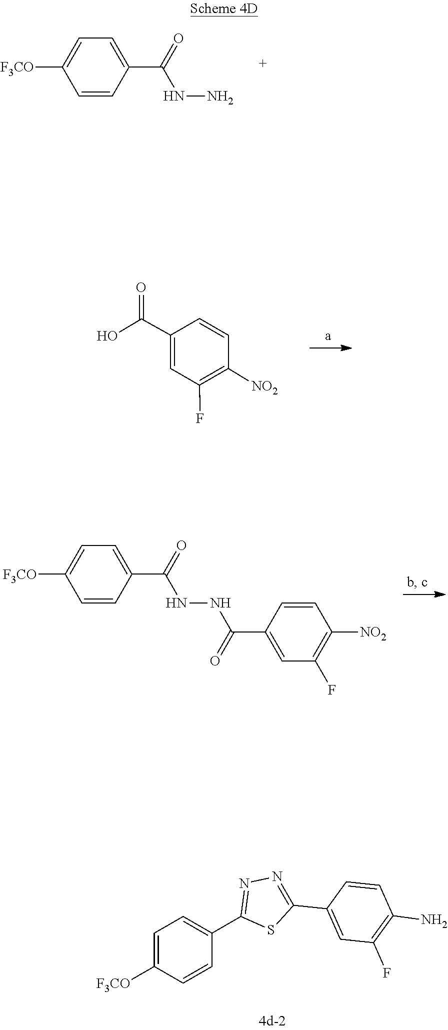 [SMILES: NNC(=O)c1ccc(OC(F)(F)F)cc1 ; O=C(O)c1ccc([N+](=O)[O-])c(F)c1 ; O=C(NNC(=O)c1ccc([N+](=O)[O-])c(F)c1)c1ccc(OC(F)(F)F)cc1 ; Nc1ccc(-c2nnc(-c3ccc(OC(F)(F)F)cc3)s2)cc1F]

Triaryl intermediates, Ar¹-Het-Ar², wherein Het is Het-H (1,2,3-triazole) can be prepared according to Scheme 4E in a two-step process. In step a, a mixture of an aniline, an acetophenone, and 4-methylbenzenesulfonohydrazide is treated with molecular iodine in a solvent such as DMSO at elevated temperature (100° C.) to afford the corresponding Ar¹-Het-Ar² nitro intermediate 4e-1. This procedure is adapted from Chen, Z.; Yan, Q.; Liu, Z.; Zhang, Y. Chem Eur. J. 2014, 20, 17635-17639. The nitro group on 4e-1 can be reduced using standard reduction conditions involving iron powder and ammonium chloride in an ethanol-water solvent mixture at 80° C. to provide triaryl intermediate 4e-2, as in step b.

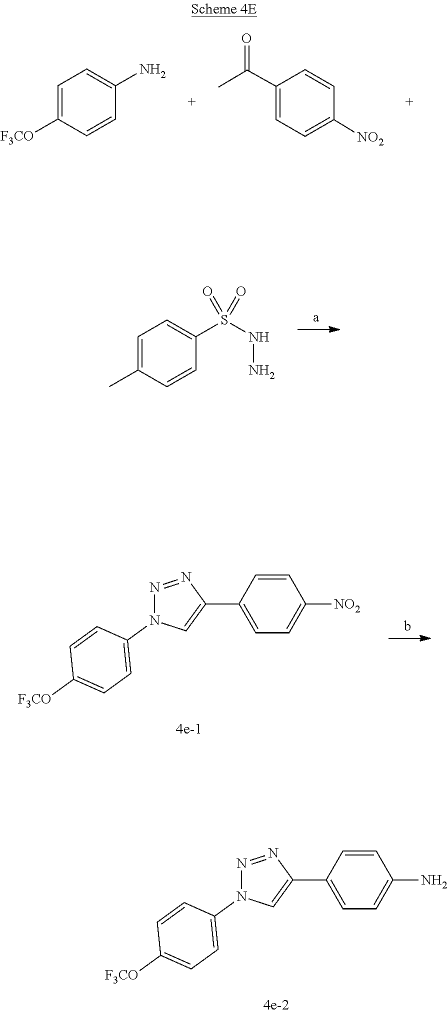 [SMILES: Nc1ccc(OC(F)(F)F)cc1 ; CC(=O)c1ccc([N+](=O)[O-])cc1 ; Cc1ccc(S(=O)(=O)NN)cc1 ; O=[N+]([O-])c1ccc(-c2cn(-c3ccc(OC(F)(F)F)cc3)nn2)cc1 ; Nc1ccc(-c2cn(-c3ccc(OC(F)(F)F)cc3)nn2)cc1]

Triaryl intermediates, Ar¹-Het-Ar², wherein Het is Het-K (1,3,4-oxadiazole) can be prepared according to Scheme 4F in a two-step process. In step a, nitrophenyltetrazole, such as 5-(4-(nitrophenyl)-2H-tetrazole, can be reacted with a boronic acid, such as (4-(trifluoromethoxy)phenyl)boronic acid, in the presence of copper(II) acetate, in the presence of a base such as pyridine, and in a solvent such as dichloromethane at room temperature to afford the corresponding Ar¹-Het-Ar² nitro intermediate 4f-1. The reaction is adapted from Li, Y.; Gao, L.-X.; Han, F.-S. Chem. Commun. 2012, 48, 2719-2721. The nitro group on 4f-1 can be reduced using standard reduction conditions involving iron powder and ammonium chloride in an ethanol-water solvent mixture at 80° C. to provide triaryl intermediate 4f-2, as in step b.

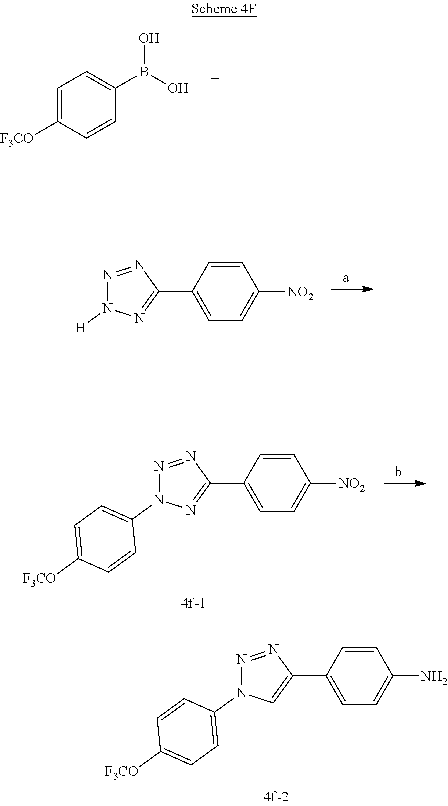 [SMILES: OB(O)c1ccc(OC(F)(F)F)cc1 ; O=[N+]([O-])c1ccc(-c2nn[nH]n2)cc1 ; O=[N+]([O-])c1ccc(-c2nnn(-c3ccc(OC(F)(F)F)cc3)n2)cc1 ; Nc1ccc(-c2cn(-c3ccc(OC(F)(F)F)cc3)nn2)cc1]

Triaryl precursor molecules, such as Ar¹-Het-X¹ or X¹—Het-Ar² wherein X¹ is Br and Het is Het-G or Het-L (imidazole) or Het-C (triazole), can be prepared via methods described in Bouchet et al., Tetrahedron 1979, 35, 1331-1338 and shown in Scheme 4G. Displacement of an aryl halide containing activating groups such as NO₂, SF₅ or SO₂CF₃ with an imidazole or triazole in the presence of a base such as potassium carbonate in a solvent such as N,N-dimethylformamide provides precursor molecules 4g-1, 4g-2, 4g-3, and 4g-4, which can be used in other reactions to afford triaryl intermediates, Ar¹-Het-Ar², wherein Het is Het-G or Het-L (imidazole) or Het-C (triazole).

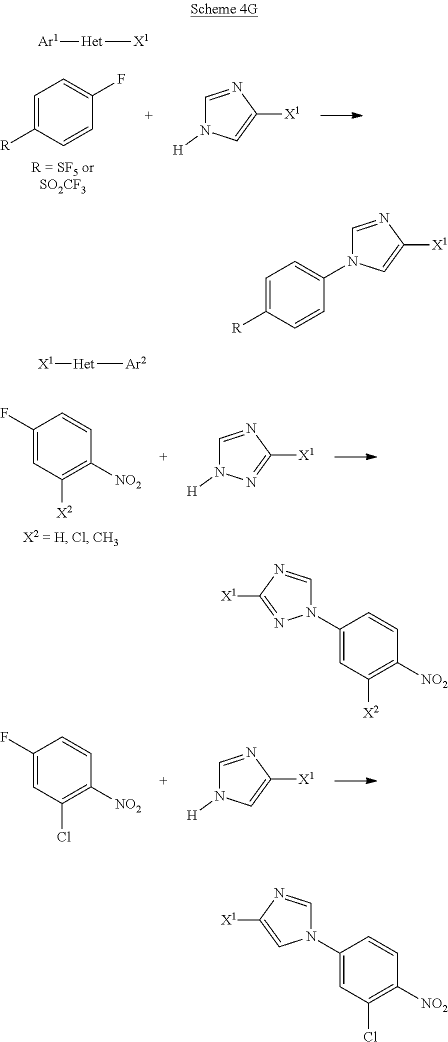

Triaryl precursor molecules, such as Ar¹—Het-X¹ or X¹—Het-Ar² wherein X¹ is Br and Het is Het-C (triazole), can be prepared via methods adapted Xia, N.; Taillefer, M. Angew. Chem. Int. Ed. 2009, 48, 337-339 and shown in Scheme 4H. Aryl halides such as 4h-1 or 4h-2 can be coupled with a bromotriazole in the presence of copper(II) acetylacetonate and acetylacetone, in the presence of a base such as cesium carbonate, and in a solvent such as N,N-dimethylformamide at elevated temperature (90-100° C.) to provide precursor molecules 4h-3 and 4h-4, which can be used in other reactions to afford triaryl intermediates, Ar¹-Het-Ar², wherein Het is Het-C (triazole).

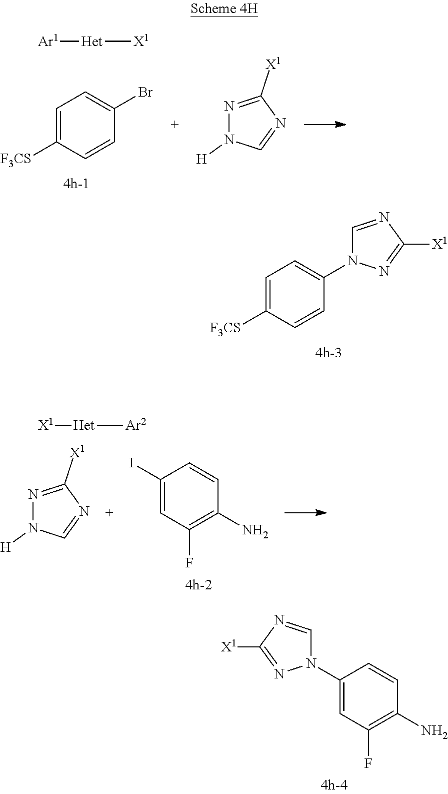

### Preparation of 1-Atom Linked Intermediates

Molecules of Formula One wherein L is a one-carbon linker, can be prepared from acid or amine intermediates described in Scheme 5 and Scheme 6, respectively. Acid precursors Ar¹-Het-Ar²-L-CO₂H, unsubstituted or mono- or di-substituted with R⁸; can be prepared as shown in the Scheme 5. Boronic esters (5-2, step a) can be prepared using Miyaura conditions from halophenyl esters (5-1). Coupling of the boronate esters with a bromo-heterocycle (5-3, step b) can be accomplished using a palladium catalyst and phosphine ligand, in the presence of a base, such as sodium bicarbonate, potassium phosphate, or cesium fluoride, in a suitable solvent system, such as dioxane/water, at temperatures from about 50° C. to about 120° C. to form triaryl ester intermediates (5-4, step c). Among palladium catalysts, tetrakis(triphenylphosphine) palladium(0) is preferred, although other well-known palladium catalysts may be used. Saponification of the ester may be achieved by using a strong base such as sodium hydroxide or lithium hydroxide in methanol or ethanol with or without tetrahydrofuran/water to furnish the desired carboxylic acid (5-5, step c).

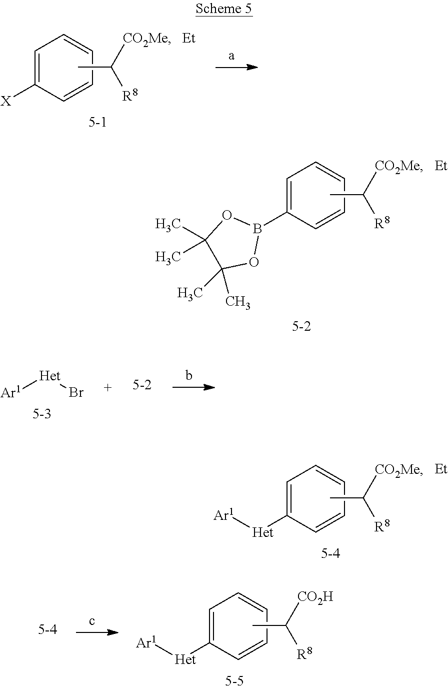

Amine precursors Ar¹-Het-Ar²-L-NH₂, unsubstituted or mono- or di-substituted with R⁸, can be prepared as shown in the Scheme 6. Halobenzyl amines (6-1) may be protected using benzyl chloroformate in the presence of a base such as triethylamine in an aprotic solvent such as dichloromethane at about −10° C. to about 10° C. to provide N-carboxybenzyl (Cbz) protected benzyl amines (6-2, step a).

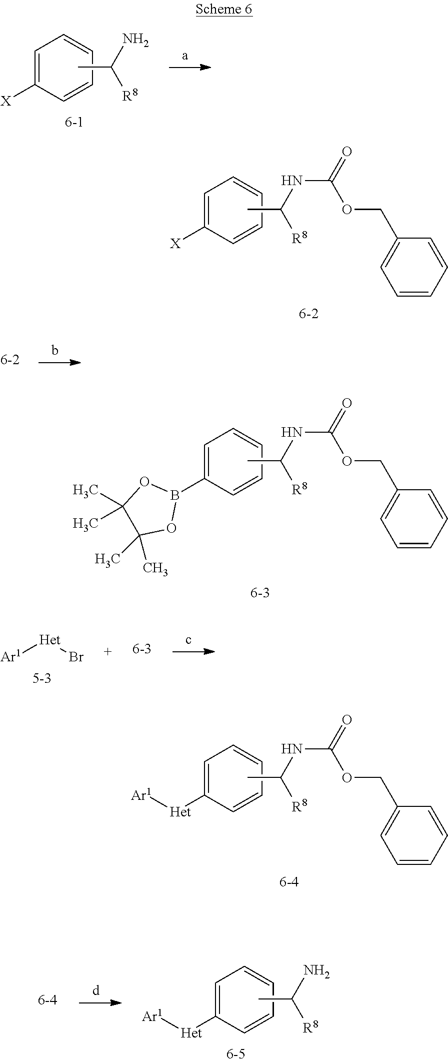

Alternatively, other N-protecting groups such as tert-butoxycarbonyl (BOC) or 9-fluorenylmethylcarbonyl (Fmoc) may be employed in step a using similar conditions described above for Cbz. The Cbz protected boronic ester 6-3 can be prepared using Miyaura conditions (step b). Coupling of the boronate esters with a bromo-heterocycle (5-3) can be accomplished using a palladium catalyst and phosphine ligand, in the presence of a base, such as sodium bicarbonate, potassium phosphate, or cesium fluoride, in a suitable solvent system, such as dioxane/water, at temperatures from about 50° C. to about 120° C. to form N-protected aminoalkylphenyl intermediates (6-4, step c). Removal of the Cbz group can be accomplished under acidic conditions with a strong acid such as hydrogen bromide, followed by free basing with a base such as sodium bicarbonate or sodium hydroxide, to furnish the free amine precursors Ar¹-Het-Ar²-L-NH₂ (6-5, step d). Similar methods could be applied to compounds wherein L is greater than 1-carbon.

### Preparation of Ethyl Linked Intermediates

Preparation of compounds wherein L is a two-atom group is described in Schemes 7 to Schemes 9. Condensation of the aldehyde (7-1, R⁹═H) (described in US 2012/0202688 A1) with reagents such as ethyl diethylphosphonoacetate or a Wittig reagent such as ethyl 2-(triphenylphosphoranylidene)propanoate) or α-substituted acetates such as ethyl 2-fluoroacetate or ethyl 2-cyanoacetate in the presence of a suitable base such as sodium hydride or n-butyl lithium in aprotic solvents such as tetrahydrofuran or diethyl ether at temperatures from about −78° C. to about 20° C. can be used to prepare acrylic esters (7-2, step a) unsubstituted or mono-substituted with R⁹ and R¹⁰. Saponification of the resultant ester may be achieved by using a strong base such as sodium hydroxide in methanol or ethanol with or without tetrahydrofuran/water to furnish the vinyl carboxylic acid (7-3, step b). In some cases the partial condensation of aldehyde (7-1, R⁹═H) may result in the isolation of the alcohol intermediate (7-4, step c) especially when R¹⁰ is electron withdrawing. Substitution of this alcohol with nucleophilic reagents such as Deoxo-Fluor® (step d) followed by saponification as described above (step e) can generate highly substituted ethyl carboxylic acids (7-5) additionally substituted with R¹¹, wherein R¹¹ is defined as R⁸ above. When the saturated linkage is preferred, the acrylate ester (7-2) can be converted to the corresponding cyclopropane (7-6, step f) unsubstituted or mono- or di-substituted with R¹²; with sulfur ylides such as those formed in situ from trimethyl sulfonium iodide in the presence of an inorganic base such as sodium hydride in a polar aprotic solvent such as dimethyl sulfoxide or tetrahydrofuran. Likewise the acrylate ester (7-2) can be reduced to the parent alkane (7-8, step h) using hydrogen gas and a palladium catalyst. Both the cyclopropane and the alkane can be hydrolyzed under basic conditions described above to generate the free carboxylic acids 7-7 (step g) and 7-9 (step i), respectively.

In a similar manner, condensation of the ketone (7-1, R⁹=alkyl) (described in WO 2011017504 A1) with either ethyl diethylphosphonoacetate or a Wittig reagent such as ethyl 2-(triphenylphosphoranylidene)propanoate or α-substituted alkyl esters such as ethyl 2-fluoroacetate or ethyl 2-cyanoacetate under similar conditions described above may generate the α-alkyl acrylate esters 7-2 or alcohols 7-4. Subsequent treatment of 7-2 or 7-4 as described above for R⁹═H may lead to either the corresponding unsaturated (7-3) or saturated (7-5, 7-7, 7-9) carboxylic acids.

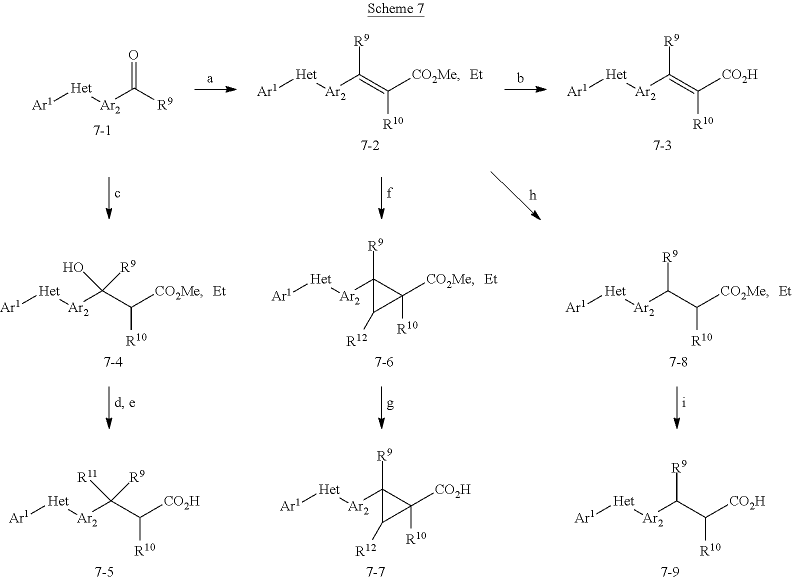

Alternatively, compounds wherein L is a 2-carbon linker may also be prepared as shown in Scheme 8. Using conditions first described by Molander et al. Org. Lett. 2007, 9, pp 203-206, coupling of a bromide Ar¹-Het-Ar²—Br (8-1, step a), with potassium (2-((tert-butoxycarbonyl)amino)ethyl)trifluoroborate in the presence of a palladium catalyst such as palladium(II) acetate, and a base such as cesium carbonate, at temperatures from about 80° C. to about 120° C., results in the formation of the corresponding 2-(tert-butoxycarbonyl)amino)ethyl derivative 8-2. Further treatment of this material with from about 1 to about 5 equivalents of an acid such as trifluoroacetic acid or hydrogen chloride, in an aprotic solvent such as dichloromethane or dioxane at temperatures from about 0° C. to about 50° C., results in the cleavage of the tert-butoxycarbonyl group and formation of the trifluoroacetic acid salt of the amine Ar¹-Het-Ar²-L-NH₂ (8-3, step b).

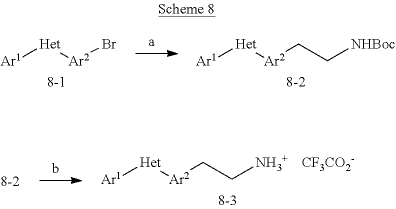

Aminoalkyl precursors Ar¹-Het-Ar²-L-NH₂, wherein L is 2-carbon atoms, mono- or di-substituted with R⁹, wherein R⁹ is defined as above; and unsubstituted or mono-substituted with R¹⁰, wherein R¹⁰ is defined as above, can be prepared as shown in Scheme 9. Halophenyl carbinols 9-1, wherein X can be selected from C₁, Br, or I, unsubstituted at R⁹ and R¹⁰ are available commercially. Carbinols 9-1 that are mono- or di-substituted at R⁹ can be prepared from the corresponding halophenyl acetate (9-I, step a) in similar fashion to that described by Shin et al. Bioorg. Med. Chem. Lett. 2008, 18, pp 4424-4427 followed by reduction with a metal hydride such as lithium aluminum hydride in an ethereal solvent such as tetrahydrofuran or diethyl ether at temperatures at or below about 0° C. Both 9-1 and 9-II may be further mono-substituted (step b or step c) with R¹⁰ via reduction to the corresponding aldehyde with a metal hydride such as diisobutylaluminum hydride and further treatment with a Grignard reagent in a similar fashion to that described by Brimble et al. Org. Lett. 2012, 14, pp 5820-5823. Carbinols 9-1 can be treated with phthalimide under Mitsunobu conditions to generate N-phthalimido intermediates 9-2 (step d). The halide can be converted into a boronic ester under Miyaura conditions to form boronate esters (9-3, step e). Coupling of the boronate esters with a bromo-heterocycle can be accomplished using a palladium catalyst, such tetrakis(triphenylphosphine) palladium(0), in the presence of a base, such as sodium bicarbonate, in a suitable solvent system, such as dioxane/water, at temperatures from about 50° C. to about 120° C. to provide N-phthalimido intermediates 9-4 (step f). Deprotection using hydrazine and methanol or other suitable solvent can furnish the amine 9-5 (step g).

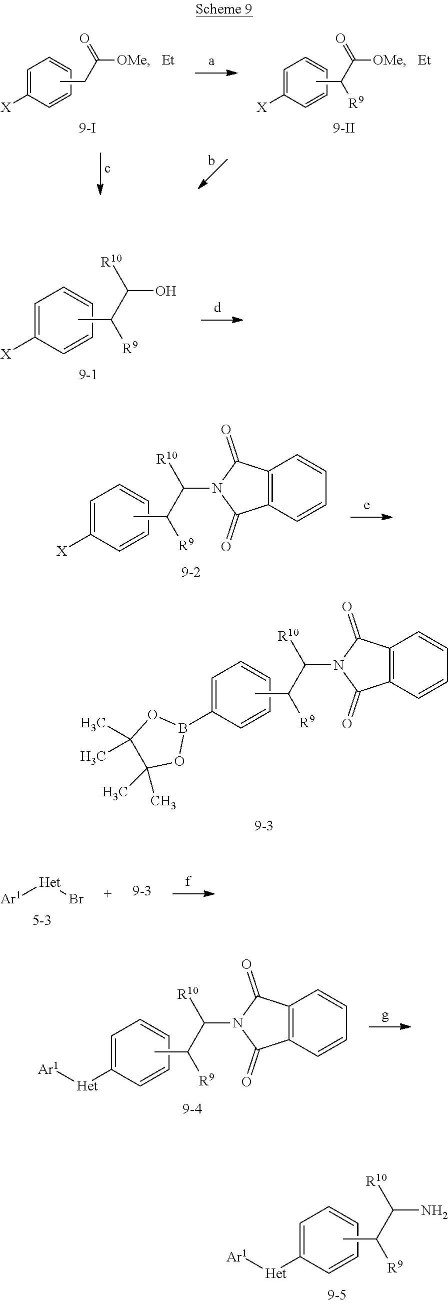

Alternatively, compounds wherein L is a 2-atom linker may also be prepared as shown in Scheme 9a. Olefination of aldehyde (7-1, R⁹═H, step a) may be achieved with methylenetriphenylphosphorane which can be prepared from methyl triphenylphosphonium iodide in the presence of a base such as sodium hydride or 1,8-diazabicycloundec-7-ene in an aprotic solvent such as tetrahydrofuran or dichloromethane at temperatures of about −78° C. to about 40° C. Further treatment of this material (9-2a) with a hydroborating reagent such as 9-borabicyclo(3.3.1)nonane in an aprotic solvent such as tetrahydrofuran followed by oxidation with an oxidant such as hydrogen peroxide can generate ethyl alcohol 9-3a (step b). Carbinols 9-3a can be treated with phthalimide under Mitsunobu conditions to generate N-phthalimido intermediates 9-5a (step c), wherein R¹⁰═H. Deprotection using hydrazine and methanol or other suitable solvent can furnish the amine 9-6a (step f). Additionally, 9-3a may be further mono-substituted (step d) with R¹⁰, wherein R¹⁰ is defined as above, via oxidation to the corresponding aldehyde under Swern conditions followed by addition of a Grignard reagent such as described above (Scheme 9). Carbinols 9-4a can be further treated with phthalimide under Mitsunobu conditions to generate N-phthalimido intermediates 9-5a (step e). Deprotection using hydrazine and methanol or other suitable solvent can furnish the amine 9-6a (step f).

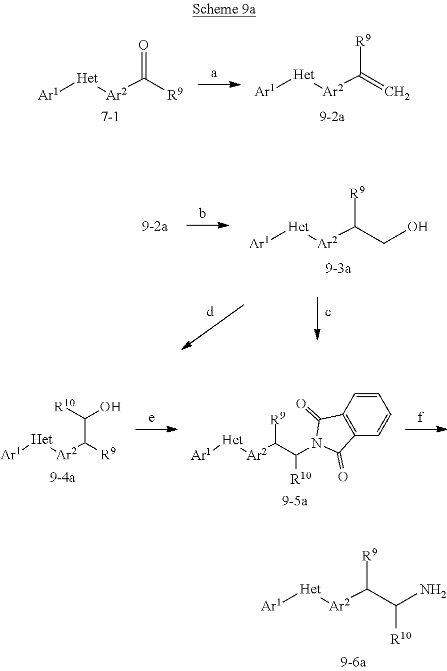

Scheme 9b outlines an alternative route for constructing analogs wherein the linker L is a two-atom linker. Copper-catalyzed arylation of 2,4-pentane-2,4-dione with 8-1 (J. Am. Chem. Soc. 2010, 132, 8273) may provide the substituted acetone intermediate 9-1b (step a). Reductive amination (step b), using any of a variety of conditions familiar to those skilled in the art, may generate amine 9-2b, which may be converted into the target molecules using conditions described previously in Scheme 2. When a linker contains a chiral center, such as with intermediate 9-2b, these intermediates may be separated into their pure isomeric forms either by means of a chiral column, or by fractional crystallization of the salt prepared from a chiral acid such as (+) and (−) tartaric acid.

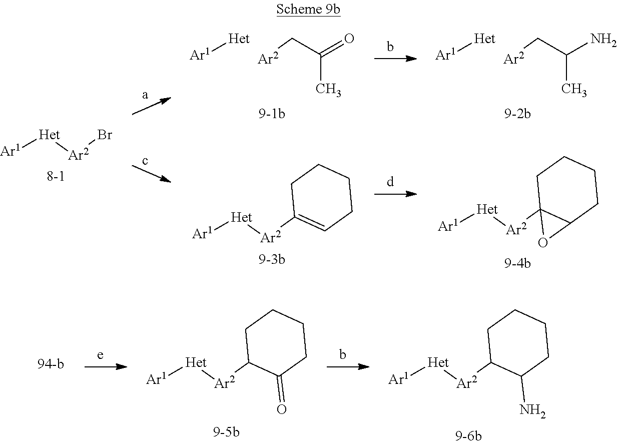

Construction of analogs wherein the ethyl linking group is part of a 6-membered ring is may also be accomplished starting from bromide 8-1. Coupling of 8-1 with 2-cyclohex-1-enyl-4,4,5,5-tetramethyl-1,3,2-dioxaborolane (Scheme 9b, step c) under standard Suzuki coupling conditions can lead to alkene 9-3b. Epoxidation with standard reagents, such as meta-chloroperoxybenzoic acid (step d), followed by acid-catalyzed rearrangement using indium trichloride (J. Org. Chem. 1998, 63, 8212) may generate ketone 9-5b. Reductive amination and conversion into the target molecules can be accomplished using conditions described above.

### Preparation of Propyl Linked Intermediates

Preparation of compounds wherein L is a three-atom group is described in Schemes 10 and 11. Aminoalkyl precursors Ar¹-Het-Ar²-L-NH₂, wherein L is 3-carbon atoms, mono- or di-substituted with R⁹, wherein R⁹ is defined as above; and unsubstituted or mono-substituted with R¹⁰, wherein R¹⁰ is defined as above; can be prepared as shown in Scheme 10. Halophenyl carbinol 10-1, wherein X is Br and R⁹ and R¹⁰ are H, is available commercially. Carbinols 10-1 that are mono- or di-substituted at R⁹ can be prepared from the corresponding halophenyl acetate (10-I, step a) in similar fashion to that described by Shin et al. Bioorg. Med. Chem. Lett. 2008, 18, pp 4424-4427, followed by reduction with a metal hydride such as lithium aluminum hydride in an ethereal solvent such as tetrahydrofuran at temperatures at or below about 0° C. Both 10-1 and 10-11 may be further mono-substituted (step b or step c) with R¹⁰ via reduction to the corresponding aldehyde with a metal hydride such as diisobutylaluminum hydride and further treatment with a Grignard reagent such as methylmagnesium bromide in a similar fashion to that described by Brimble et al. Org. Lett. 2012, 14, pp 5820-5823; Carbinols 10-1 can be treated with phthalimide under Mitsunobu conditions to generate N-phthalimido intermediates 10-2 (step d).

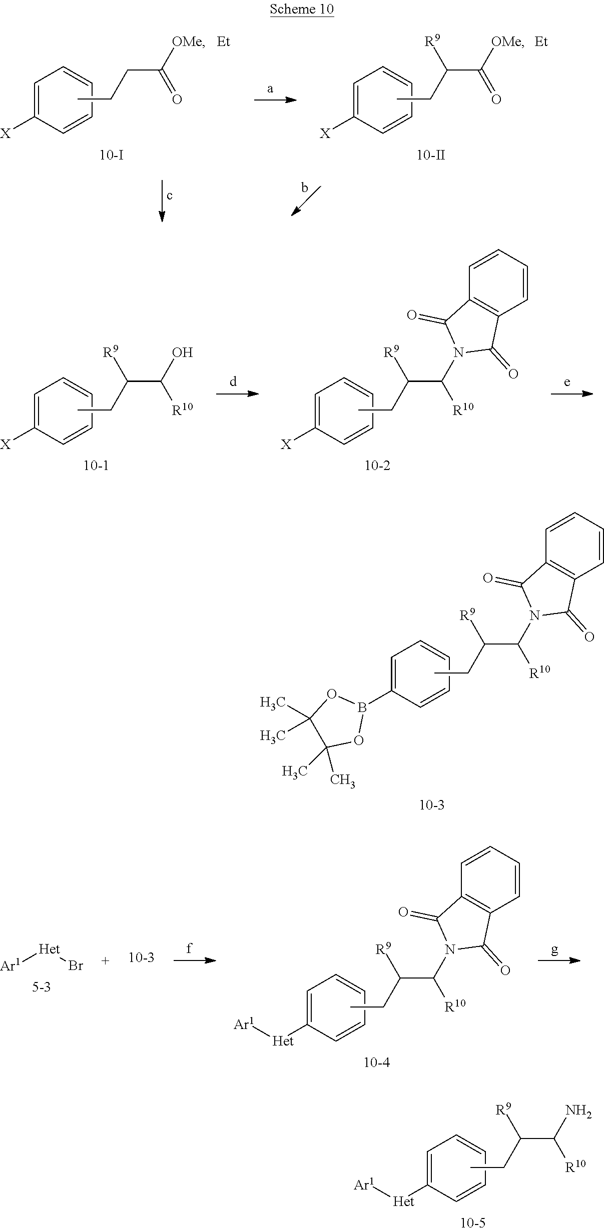

The halide can be converted into a boronic ester under Miyaura conditions (10-3, step e). Coupling of the boronate esters with a bromo-heterocycle can be accomplished using a palladium catalyst, such tetrakis(triphenylphosphine) palladium(0), in the presence of a base, such as sodium bicarbonate, in a suitable solvent system, such as dioxane/water, at temperatures from about 50° C. to about 120° C. to provide N-phthalimido intermediates 10-4 (step f). Deprotection using hydrazine and methanol or other suitable solvent can furnish the amine 10-5 (step g).

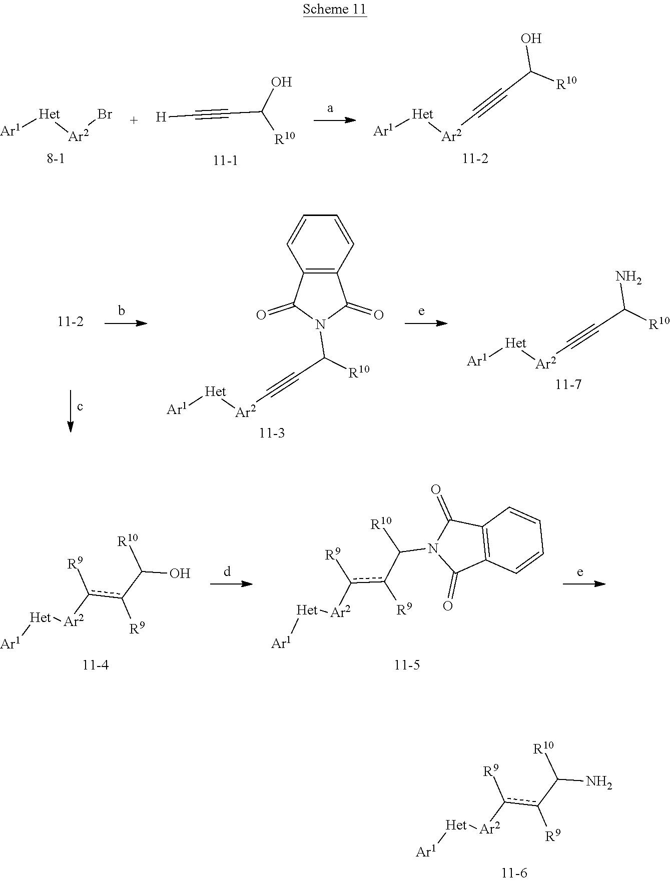

Alternatively, compounds wherein L is a 3-atom linker may also be prepared as shown in Scheme 11. Bromide Ar¹-Het-Ar²—Br (8-1) can be coupled with an appropriate alkynyl alcohol (11-1, step a) unsubstituted or mono-substituted with R¹⁰, wherein R¹⁰ is defined as above; in the presence of a palladium catalyst such as bistriphenylphosphine dichloropalladium(II), copper(I) iodide, and a base such as triethylamine, at temperatures from about 50° C. to about 120° C., to generate the corresponding alkynyl alcohol derivatives 11-2. The resultant carbinols 11-2 can be treated with phthalimide under Mitsunobu conditions to generate N-phthalimido intermediates 11-3 (step b) which can be converted to amine (11-7, step e) using hydrazine and methanol or other suitable solvent. Carbinols 11-2 can be reduced using a transition metal catalyst, such as palladium under an atmosphere of hydrogen to provide alkenyl or fully saturated alkyl carbinols 11-4 unsubstituted at R¹⁰. Additionally, carbinols 11-2 can be treated with a metal hydride such as lithium aluminum hydride to provide the (E)-alkenyl carbinol 11-4. Likewise, carbinol 11-2 may be protected with a protecting group such as tert-butyl diphenyl silane, and treated with a hydrometallation reagent such as Schwartz' reagent followed by an electrophile quench, with, for example, elemental iodine or N-bromosuccinimide (NBS). Alternatively, the carbinol 11-2 may be treated with a transmetallation reagent such as pinacol diboron for further use in transition metal-catalyzed coupling reactions, such as Suzuki or Negishi, to prepare carbinols 11-4 mono- or di-substituted with R⁹, wherein R⁹ is defined as above (step c). Following deprotection, the resultant carbinols 11-4 can be treated with phthalimide under Mitsunobu conditions to generate N-phthalimido intermediates 11-5 (step d) which can be converted to an amine (11-6, step e) using hydrazine and methanol or other suitable solvent.

### Preparation of Butyl Linked Intermediates

Compounds wherein L is a 4-atom linker may be prepared as shown in Scheme 12. Bromide Ar¹-Het-Ar²—Br (8-1) can be coupled with an appropriate alkynyl alcohol (12-1, step a) unsubstituted or mono-substituted with R¹⁰, wherein R¹⁰ is defined as above; mono- or di-substituted with R⁹, wherein R⁹ is defined as above; in the presence of a palladium catalyst such as bistriphenylphosphine dichloropalladium, copper(I) iodide, and a base such as triethylamine, at temperatures from about 50° C. to about 120° C., to generate the corresponding alkynyl alcohol derivatives 12-2. The resultant carbinols 12-2 can be treated with phthalimide under Mitsunobu conditions (step b) to generate N-phthalimido intermediates 12-3 which can be converted to an amine (12-7, step e) using hydrazine and methanol or other suitable solvents. Carbinols 12-2 can be reduced using a transition metal catalyst, such as palladium under an atmosphere of hydrogen to provide alkenyl or fully saturated alkyl carbinols 12-4 (step c) unsubstituted at R¹³. Additionally, carbinols 12-2 can be treated with a metal hydride such as lithium aluminum hydride to provide the (E)-alkenyl carbinols 12-4 (step c). Likewise, carbinol 12-2 may be protected with a protecting group such as tert-butyl diphenyl silane, and treated with a hydrometallation reagent such as Schwartz' reagent followed by an electrophile quench, with, for example, elemental iodine or NBS. Alternatively the carbinol 12-2 may be treated with a transmetallation reagent such as pinacol diboron for further use in transition metal-catalyzed coupling reactions, such as Suzuki or Negishi, to prepare carbinols 12-4 mono- or di-substituted with R¹³, wherein R¹³ is defined as R⁸ above (step c).

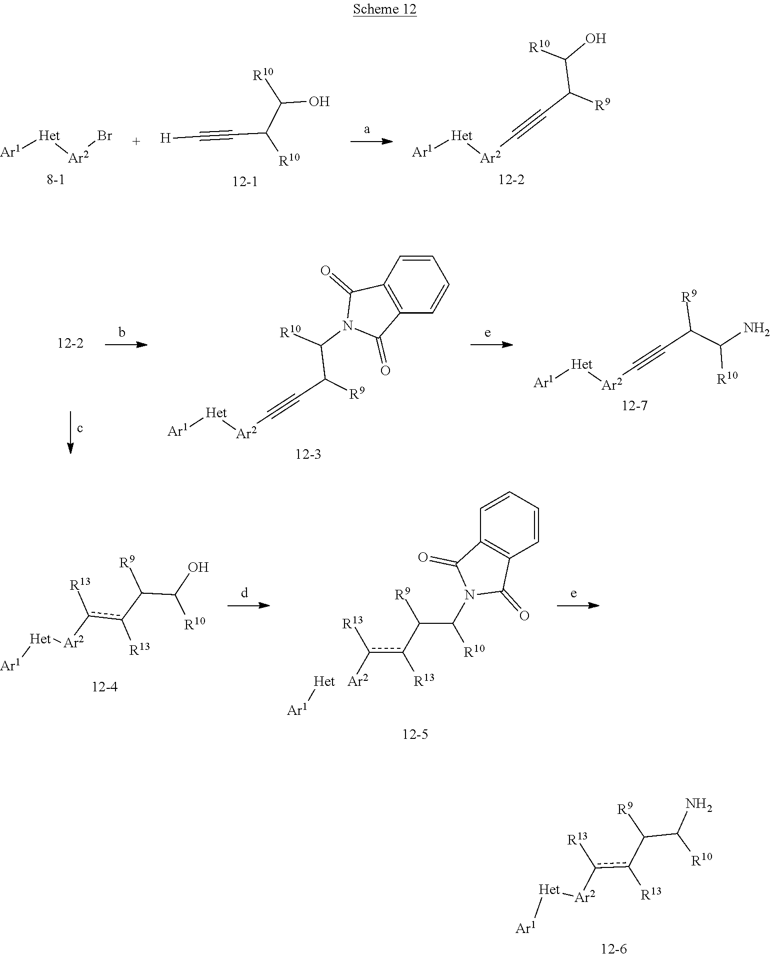

Following deprotection, the resultant carbinols 12-4 can be treated with phthalimide under Mitsunobu conditions (step d) to generate N-phthalimido intermediates 12-5 which can be converted to an amine (12-6, step e) using hydrazine and methanol or other suitable solvent.

### Preparation of Substituted Thiobiurets

2-Imino 1,3-thiazolin-4-ones (3-1) may be further functionalized using a variety of conditions. When treated with Selectfluor® and 9-fluorenone in anhydrous acetonitrile (J. Am. Chem. Soc. 2013, 135, 17494), molecules having the formula 3-1 may be converted into the mono-fluoro analogs (13-1).

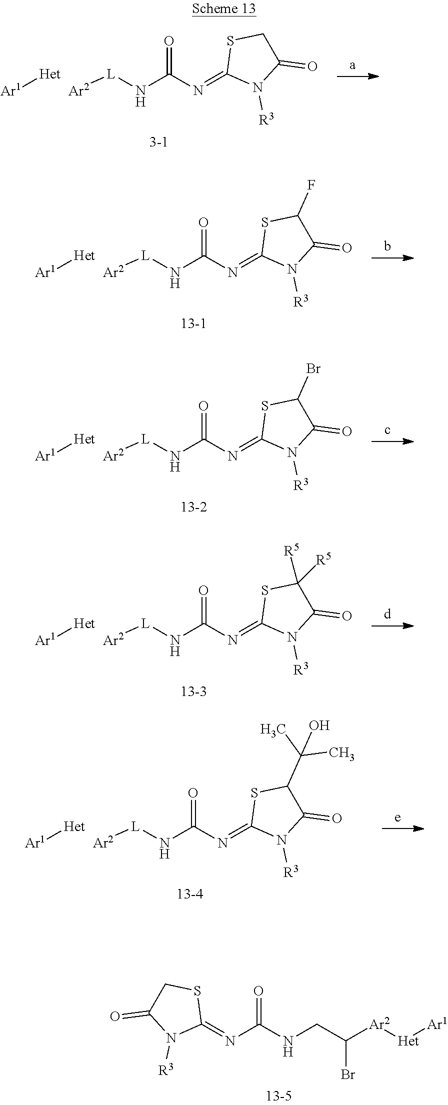

Treatment with molecular bromine in a aprotic solvent such as dichloromethane at from about 0° C. to about 30° C. (step b) may result in mono-bromination on the thiazolinone ring (13-2). Alkylation (step c), using at least 2 equivalents of an alkylating agent R⁵-1 and a strong base such as sodium hydride or lithium diisopropylamide in a polar aprotic solvent such as dimethylformamide or tetrahydrofuran may lead to a di-alkylated product (13-3). Treatment with a ketone or an aldehyde and an inorganic base such as potassium carbonate or cesium carbonate may result in the formation of a carbinol (13-4). For analogs of compounds of the formula 3-1 wherein L is a —CH₂CH₂— group, free-radical bromination using N-bromosuccinimide and a free radical initiator such as azobis (isobutyronitrile) in carbon tetrachloride at about 30° C. to about 77° C. may lead to the mono-brominated product (13-5) wherein the bromine is incorporated into the ethyl linker.

The compounds of Formula One disclosed herein may be synthesized using methodology familiar to one skilled in the art, as well methods given in Fischer at al., United States Patent Application Publication 20140274688 A1 and Baum, et al., WO2016033025 A1, using known or commercially available starting materials.

### EXAMPLES

These examples are for illustration purposes and are not to be construed as limiting the disclosure to only the embodiments disclosed in these examples.

Starting materials, reagents, and solvents that were obtained from commercial sources were used without further purification. Anhydrous solvents were purchased as Sure/Sea™ from Aldrich and were used as received. Melting points were obtained on a Thomas Hoover Unimelt capillary melting point apparatus or an OptiMelt Automated Melting Point System from Stanford Research Systems and are uncorrected. Examples using “room temperature (or rt)” were conducted in climate controlled laboratories with temperatures ranging from about 20° C. to about 24° C. Molecules are given their known names, named according to naming programs within ISIS Draw, ChemDraw or ACD Name Pro. If such programs are unable to name a molecule, the molecule is named using conventional naming rules. ¹H NMR spectral data are in ppm (δ) and were recorded at 300, 400 or 600 MHz, and ¹³C NMR spectral data are in ppm (δ) and were recorded at 75, 100 or 150 MHz, unless otherwise stated.

### Example 1: Preparation of 2-(4-(1-(4-(trifluoromethoxy)phenyl)-1H-1,2,4-triazol-3-yl)phenyl)acetonitrile (C1)

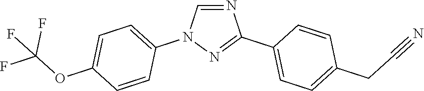 [SMILES: N#CCc1ccc(-c2ncn(-c3ccc(OC(F)(F)F)cc3)n2)cc1]

To a reaction flack were added (4-(cyanomethyl)phenyl)boronic acid (2.55 g, 15.8 mmol), 3-bromo-1-(4-(trifluoromethoxy)phenyl)-1H-1,2,4-triazole (prepared as in Fischer at al., United States Patent Application Publication 20140274688 A1; 3.9 g, 12.66 mmol) and sodium carbonate (2.68 g, 25.3 mmol), followed by tetrakis(triphenylphosphine)palladium(0) (Pd(PPh₃)₄; 0.731 g, 0.633 mmol). The flask was evacuated and back-filled with nitrogen (3×) before dioxane (45 mL) and water (14.9 mL) were added. The reaction mixture was then heated to 95° C. for 9 hours (h). The reaction mixture was cooled to room temperature (rt) and was diluted with ethyl acetate (EtOAc) and water. The organic layer was separated and washed with brine, dried over Na₂SO₄, filtered and concentrated to a yellow semi-solid. The unpurified material was adhered to Celite® with EtOAc. Purification by silica gel column chromatography eluting with a 0-50% EtOAc-hexanes gradient over 20 minutes (min) provided the title compound as an off-white solid (3.92 g, 88%): ¹H NMR (400 MHz, CDCl₃) δ 8.58 (s, 1H), 8.28-8.16 (m, 2H), 7.90-7.74 (m, 2H), 7.50-7.43 (m, 2H), 7.43-7.35 (m, 2H), 3.83 (s, 2H); ¹⁹F NMR (376 MHz, CDCl₃) δ −58.02; ESIMS m/z 345 ([M+H]⁺).

### Example 2: Preparation of methyl 3-cyano-3-(4-(1-(4-(trifluoromethoxy)phenyl)-1H-1,2,4-triazol-3-yl)phenyl)propanoate (C₂)

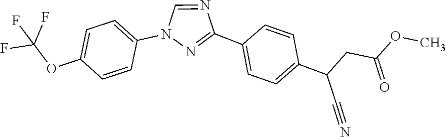 [SMILES: COC(=O)CC(C#N)c1ccc(-c2ncn(-c3ccc(OC(F)(F)F)cc3)n2)cc1]

2-(4-(1-(4-(Trifluoromethoxy)phenyl)-1H-1,2,4-triazol-3-yl)phenyl)acetonitrile (C1) (250 mg, 0.726 mmol) was stirred in N,N-dimethylformamide (DMF) at 0° C. Sodium hydride (NaH, 60% in mineral oil; 31.9 mg, 0.799 mmol) was added portionwise, and the reaction mixture was allowed to stir for 15 min. Methyl 2-bromoacetate (0.087 mL, 0.919 mmol) was added, and the reaction mixture was allowed to stir and warm to rt overnight. The reaction was poured onto ice. 1 Normal (N) hydrochloric acid (HCl) was added, and the aqueous phase was extracted with diethyl ether (Et₂O). The ether layer was adsorbed onto silica gel. Purification via flash chromatography (silica, 0-40% EtOAc/Hexanes) yielded the title compound as a white solid (70 mg, 23%): ¹H NMR (500 MHz, CDCl₃) δ 8.58 (s, 1H), 8.24-8.20 (m, 2H), 7.83-7.78 (m, 2H), 7.52-7.47 (m, 2H), 7.42-7.37 (m, 2H), 4.37 (dd, J=8.1, 6.7 Hz, 1H), 3.74 (s, 3H), 3.08 (dd, J=16.6, 8.1 Hz, 1H), 2.90 (dd, J=16.6, 6.8 Hz, 1H); ¹⁹F NMR (471 MHz, CDCl₃) δ −58.02; ESIMS m/z 417 ([M+H]⁺).

### Example 3: Preparation of 3-cyano-3-(4-(1-(4-(trifluoromethoxy)phenyl)-1H-1,2,4-triazol-3-yl)phenyl)propanoic acid (C3)

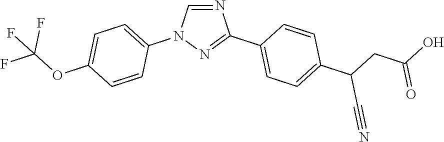 [SMILES: N#CC(CC(=O)O)c1ccc(-c2ncn(-c3ccc(OC(F)(F)F)cc3)n2)cc1]

To methyl 3-cyano-3-(4-(1-(4-(trifluoromethoxy)phenyl)-1H-1,2,4-triazol-3-yl)phenyl)propanoate (C2; 500 mg, 1.201 mmol) suspended in methanol (5 mL) was added 2 N aqueous sodium hydroxide (NaOH; 0.721 mL, 1.44 mmol). The clear reaction mixture was stirred overnight under nitrogen at rt. The reaction mixture was made acidic with a few drops of 2 N HCl. The solvents were removed in vacuo, and the residue was dried under high vacuum. The title compound was isolated as a white solid (480 mg, 97%): ¹H NMR (400 MHz, DMSO-d₆) δ 12.80 (s, 1H), 9.43 (s, 1H), 8.17-8.03 (m, 4H), 7.62 (dd, J=8.8, 7.1 Hz, 4H), 4.56 (dd, J=8.8, 5.9 Hz, 1H), 3.06 (dd, J=16.8, 8.7 Hz, 1H), 2.92 (dd, J=16.9, 6.0 Hz, 1H); ¹⁹F NMR (376 MHz, DMSO-d₆) δ −56.96; ESIMS m/z 403 ([M+H]⁺).

### Example 4: Preparation of 3-cyano-3-(4-(1-(4-(trifluoromethoxy)phenyl)-1H-1,2,4-triazol-3-yl)phenyl)propanoyl azide (C4)

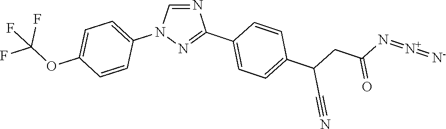 [SMILES: N#CC(CC(=O)N=[N+]=[N-])c1ccc(-c2ncn(-c3ccc(OC(F)(F)F)cc3)n2)cc1]

To a stirred mixture of 3-cyano-3-(4-(1-(4-(trifluoromethoxy)phenyl)-1H-1,2,4-triazol-3-yl)phenyl)propanoic acid (C3; 68 mg, 0.169 mmol) in 1,2-dichloroethane (DCE; 3 mL) were added sequentially oxalyl chloride (0.148 mL, 1.69 mmol) and DMF (1 drop). There was immediate gas evolution, the slurry became light yellow in color, and the mixture went into solution within a few minutes. After stirring for 1 h, the solvent was concentrated under a stream of nitrogen to give an orange solid/foam. The residue was taken up in tetrahydrofuran (THF; 2 mL) and the solution was added to a stirred solution of sodium azide (54.9 mg, 0.845 mmol) in THF-water (2:1; 3 mL) at rt. After ˜30 min at room temperature, the reaction mixture was diluted with water. The mixture was extracted with EtOAc (3×). The combined organic extracts were dried over sodium sulfate (Na₂SO₄). The solvent was evaporated under a stream of nitrogen overnight. The title compound was isolated as a thick orange oil which was used without further purification (68 mg, 94%); ESIMS m/z 428 ([M+H]⁺).

### Example 5: Preparation of (Z)-1-(3-(5-chloro-2-(trifluoromethyl)phenyl)-4-oxothiazolidin-2-ylidene)-3-(2-cyano-2-(4-(1-(4-(trifluoromethoxy)phenyl)-1H-1,2,4-triazol-3-yl)phenyl)ethyl)urea (H8)

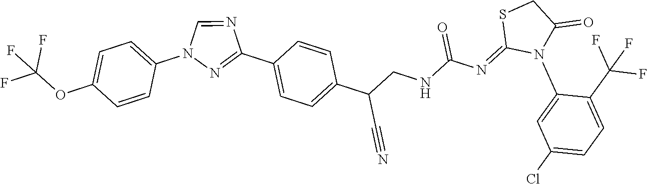 [SMILES: N#CC(CNC(=O)/N=C1\SCC(=O)N1c1cc(Cl)ccc1C(F)(F)F)c1ccc(-c2ncn(-c3ccc(OC(F)(F)F)cc3)n2)cc1]

To 3-cyano-3-(4-(1-(4-(trifluoromethoxy)phenyl)-1H-1,2,4-triazol-3-yl)phenyl)propanoyl azide (C4; 50 mg, 0.117 mmol) and 3-(5-chloro-2-(trifluoromethyl)phenyl)-2-iminothiazolidin-4-one (C5; 41.4 mg, 0.14 mmol) in a vial were diluted with acetonitrile (3 mL) and heated to 75° C. for 30 min. The reaction mixture was cooled to rt and loaded onto silica. Purification via silica gel flash column chromatography (EtOAc-hexanes:dichloromethane (DCM) (1:1)) afforded the title compound as an off-white foam (24 mg, 28%).

### Example 6: Preparation of 2,2,2-trifluoro-1-(4-methoxy-2-nitrophenyl)ethyl methanesulfonate (C6)

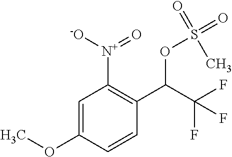

Step 1—Preparation of 2, 2, 2-trifluoro-1-(4-methoxy-2-nitrophenyl)ethan-1-ol (C7): To 4-methoxy-2-nitrobenzaldehyde (10 g, 55.2 mmol) in THF (100 mL) were added trimethyl(trifluoromethyl)silane (13 mL, 82.8 mmol) and cesium fluoride (CsF; 1.67 g, 11.0 mmol) and the reaction mixture was stirred at 0° C. under nitrogen for 3 h. After 3 h, concentrated hydrochloric acid (10 mL, 155 mmol) was added, and the reaction mixture was stirred at rt for 30 min. The reaction mixture was diluted with water (100 mL) and was extracted with DCM (2×100 mL). The organic layer was dried over anhydrous Na₂SO₄, filtered and concentrated under reduced pressure to afford the unpurified title compound as yellow oil which was used in the next step without any purification (16 g): ¹H NMR (400 MHz, CDCl₃) δ 7.83 (d, J=8.8 Hz, 1H), 7.51 (d, J=2.8 Hz, 1H), 7.24-7.21 (m, 1H), 6.05-6.03 (m, 1H), 3.90 (s, 3H), 3.76-3.72 (m, 1H).

Step 2—Preparation of 2,2,2-trifluoro-1-(4-methoxy-2-nitrophenyl)ethyl methanesulfonate (C₆): To a solution of 2, 2, 2-trifluoro-1-(4-methoxy-2-nitrophenyl)ethan-1-ol (C7; 16 g, 63.7 mmol) in DCM (160 mL) was added diisopropylethylamine (DIPEA; 22 mL, 128 mmol), and the reaction mixture was cooled to 0° C. Methanesulfonyl chloride (6.1 mL, 79.7 mmol) was added dropwise at 0° C., and the reaction mixture was stirred at rt for 1 h. The reaction mixture was diluted with water (100 mL) and was extracted with DCM (2×50 mL). The organic layer was dried over anhydrous Na₂SO₄, filtered and concentrated under reduced pressure. The residue was purified by silica gel column chromatography (100-200 mesh) eluting with 5-20% EtOAc in petroleum ether. The title compound was isolated as a red oil (8 g, 61% over 2 steps): ¹H NMR (300 MHz, CDCl₃) δ 7.78 (d, J=8.7 Hz, 1H), 7.64 (d, J=2.7 Hz, 1H), 7.29-7.26 (m, 1H), 6.97-6.95 (m, 1H), 3.92 (s, 3H), 3.12 (s, 3H).

### Example 7: Preparation of 5-methoxy-2-(2,2,2-trifluoroethyl)aniline (C₈)

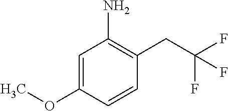

To a solution of 2,2,2-trifluoro-1-(4-methoxy-2-nitrophenyl)ethyl methanesulfonate (C6; 12 g, 36.5 mmol) in methanol (100 mL) was added Pd(OH)₂ (1.3 g) and the reaction mixture was stirred under hydrogen (60 psi) in a parr-shaker for 16 h. The reaction mixture was cooled to rt, filtered through a pad of diatomaceous earth, washed with EtOH and the filtrate was concentrated under reduced pressure. The crude product was purified by column chromatography (silica gel 100-200 mesh) eluting with 10-15% EtOAc in petroleum ether to afford 7 g (94%) of aniline intermediate as an off-white solid; ¹H NMR (300 MHz, CDCl₃) δ 7.08-7.06 (m, 1H), 6.51-6.40 (m, 2H), 6.00-5.20 (br s, 2H), 3.70 (s, 3H), 3.50-3.47 (m, 2H); ESIMS m/z 206.15 [(M+H)*].

The following compounds were prepared in accordance to the procedure in Examples 6 and 7.

### 5-Methyl-2-(2,2,2-trifluoroethyl)aniline (C9)

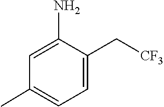

5-Methyl-2-(2,2,2-trifluoroethyl)aniline was prepared as above from 4-methyl-2-nitrobenzaldehyde.

All anilines were further converted to thiazolidinone intermediates via previously reported conditions (U.S. Pat. No. 9,029,560 B2).

### Example 8: Preparation of 3-(2-(difluoromethyl)phenyl)-2-iminothiazolidin-4-one (C10)

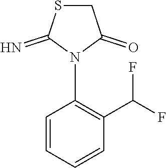 [SMILES: N=C1SCC(=O)N1c1ccccc1C(F)F]

The title compound was prepared using previously reported conditions (U.S. Pat. No. 9,029,560 B2) and was isolated as a brown solid (826 mg, 49%): ¹H NMR (500 MHz, DMSO-d₆) δ 9.33 (s, 1H), 7.77-7.56 (m, 3H), 7.35 (d, J=7.9 Hz, 1H), 6.85 (t, J=54.4 Hz, 1H), 4.28-4.10 (m, 2H); HRMS-ESI (m/z) [M+H]⁺ calcd for C₁₀H₈F₂N₂OS, 242.0325; found 242.0325.

The following compound was prepared in accordance to the procedure in Example 8.

### 3-(2-(1,1-Difluoroethyl)phenyl)-2-iminothiazolidin-4-one (C11)

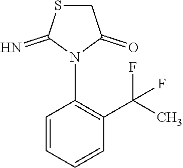 [SMILES: CC(F)(F)c1ccccc1N1C(=N)SCC1=O]

The title compound was isolated as a brown oil (110 mg, 32%): ¹H (500 MHz, CDCl₃) δ 7.65 (d, J=7.4 Hz, 1H), 7.61-7.51 (m, 2H), 7.23-7.14 (m, 1H), 4.18-3.96 (m, 2H), 1.90 (t, J=18.7 Hz, 3H); HRMS-ESI (m/z) [M+H]⁺ calcd for C₁₁H₁₀F₂N₂OS, 256.0482; found 256.0479.

### Example 9: Preparation of 2-chloro-N-(2-(difluoromethyl)phenyl)acetamide (C12)

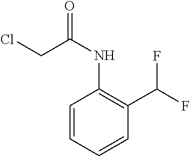 [SMILES: O=C(CCl)Nc1ccccc1C(F)F]

The title compound was prepared using previously reported conditions (U.S. Pat. No. 9,029,560 B2) and was isolated as an oil (5.01 g, 86%): ¹H NMR (400 MHz, CDCl₃) δ 8.83 (s, 1H), 8.13 (d, J=8.2 Hz, 1H), 7.57-7.49 (m, 1H), 7.44 (d, J=8.2 Hz, 1H), 7.31-7.22 (m, 2H), 6.69 (t, J=54.8 Hz, 1H), 4.23 (s, 2H); EIMS m/z 219.

The following compound was prepared in accordance to the procedure in Example 9.

### 2-Chloro-N-(2-(1,1-difluoroethyl)phenyl)acetamide (C13)

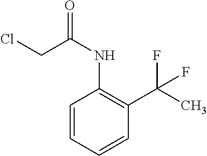

The title compound was isolated as an oil which was carried forward with no further manipulation (283 mg, 44%): EIMS m/z 233.

### Example 10: Preparation of 2-(difluoromethyl)aniline (C14)

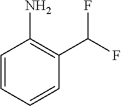

To a solution of 1-(difluoromethyl)-2-nitrobenzene (C15; 6.34 g, 36.6 mmol) and nickel(II) chloride (4.75 g, 36.6 mmol) in dry methanol (183 mL) was added sodium borohydride (2.77 g, 73.2 mmol) in a dry 1 L round-bottomed flask under a nitrogen atmosphere. The reaction mixture was stirred at 0° C. for 3 h. The solution was filtered and concentrated under vacuum. The resulting residue was dissolved in DCM and washed with water. The organic layer was dried over Na₂SO₄ and concentrated under vacuum. The title compound was isolated as a brown oil (3.23 g, 52%): ¹H NMR (400 MHz, CDCl₃) δ 7.29-7.19 (m, 2H), 6.76 (m, 2H), 6.74-6.42 (m, 1H), 4.07 (s, 2H); EIMS m/z 143.

### Example 11: Preparation of 1-(difluoromethyl)-2-nitrobenzene (C15)

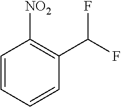 [SMILES: O=[N+]([O-])c1ccccc1C(F)F]

To a solution of 2-nitrobenzaldehyde (1 g, 6.62 mmol) in dry DCM (33.1 mL) was added deoxofluor (4.88 mL, 13.2 mmol) in a dry 100 mL round-bottomed flask under a nitrogen atmosphere. The reaction mixture was stirred at 0° C. for 18 h. The reaction mixture was slowly poured into a solution of saturated aqueous sodium bicarbonate and stirred. The layers were separated, and the aqueous layer was extracted twice with DCM. The combined organic extracts were dried over Na₂SO₄ and concentrated under vacuum. Purification by flash chromatography (silica gel) eluting with a 0-10% acetone in hexanes gradient afforded the title compound as a yellow oil (1.1 g, 86%): ¹H NMR (400 MHz, CDCl₃) δ 8.16 (dd, J=8.2, 1.2 Hz, 1H), 7.91 (dd, J=7.9, 1.4 Hz, 1H), 7.79 (td, J=7.8, 1.4 Hz, 1H), 7.73-7.59 (m, 1H), 7.57-7.23 (m, 1H); EIMS m/z 173.

### Example 12: Preparation of 2-(1,1-difluoroethyl)aniline (C16)

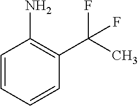

To a solution of sodium acetate (1.36 g, 16.6 mmol) and hydroxylamine hydrochloride (0.768 g, 11.1 mmol) in dry methanol (55.2 mL) was added 2-(1,1-difluoroethyl)-N-(diphenylmethylene)aniline (C17; 1.78 g, 5.52 mmol) in a dry 200 mL round-bottomed flask under a nitrogen atmosphere. The reaction mixture was stirred at 23° C. for 18 h. The reaction mixture was concentrated under vacuum and diluted with DCM. The solution was washed with water. The organic layer was dried over Na₂SO₄ and concentrated under vacuum. Purification by flash chromatography (silica gel, twice) eluting with a 0-20% EtOAc in hexanes gradient and then a 0-20% acetone in hexanes gradient afforded the title compound as a yellow oil (265 mg, 15%): EIMS m/z 157.

### Example 13: Preparation of 2-(1,1-difluoroethyl)-N-(diphenylmethylene)aniline (C17)

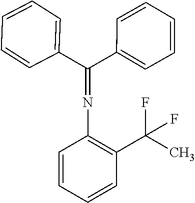 [SMILES: CC(F)(F)c1ccccc1N=C(c1ccccc1)c1ccccc1]

1-Bromo-2-(1,1-difluoroethyl)benzene (C18; 1.62 g, 7.33 mmol) was added to a solution of sodium tert-butoxide (1.06 g, 11.0 mmol), tris(dibenzylideneacetone)dipalladium(0) (Pd₂(dba)₃; 0.336 g, 0.366 mmol), (oxybis(2,1-phenylene))bis(diphenylphosphine) (0.395 g, 0.733 mmol) and diphenylmethanimine (1.60 mL, 9.53 mmol) in dry toluene (14.7 mL) in a dry, 20 mL microwave vial under a nitrogen atmosphere. The reaction mixture was stirred at 100° C. for 3 h in a Biotage® microwave reactor. The reaction mixture was diluted with DCM and washed with water. The organic layer was dried over Na₂SO₄ and concentrated under vacuum. Purification by flash chromatography (silica gel) eluting with a 0-20% EtOAc in hexanes gradient afforded the title compound as an orange oil (1.78 g, 68%): 1H NMR (500 MHz, CDCl₃) δ 7.78 (d, J=7.0 Hz, 1H), 7.53-7.44 (m, 2H), 7.41 (t, J=7.4 Hz, 2H), 7.28-7.23 (m, 3H), 7.22-7.17 (m, 2H), 7.04 (t, J=7.6 Hz, 1H), 6.95 (t, J=7.6 Hz, 1H), 6.33-6.27 (m, 1H), 2.14 (t, J=18.8 Hz, 3H); HRMS-ESI (m/z) [M+H]⁺ calcd for C₂₁H₁₇F₂N, 321.1329; found 321.1333.

### Example 14: Preparation of 1-bromo-2-(1,1-difluoroethyl)benzene (C18)

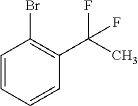

To a solution of 1-(2-bromophenyl)ethanone (1.36 mL, 10.1 mmol) and 4-tert-butyl-2,6-dimethylphenylsulfur trifluoride (5.03 g, 20.1 mmol) in dry DCM (20.1 mL) was added pyridine hydrofluoride (1.23 mL, 13.7 mmol) in a dry 50 mL Fisher Brand polypropylene tube. The reaction mixture was stirred at 23° C. for 18 h. The reaction mixture was added slowly to a solution of saturated aqueous sodium bicarbonate at 0° C. The mixture was extracted with DCM. The organic layer was dried with Na₂SO₄ and concentrated under vacuum. Purification by Kugelrohr distillation provided the title compound as a colorless oil (2.72 g, 100%): ¹H NMR (500 MHz, CDCl₃) δ 7.65-7.57 (m, 2H), 7.35 (t, J=7.6 Hz, 1H), 7.25 (td, J=7.6 Hz, 1H), 2.04 (t, J=18.4 Hz, 3H); ¹⁹F NMR (471 MHz, CDCl₃) δ −87.32.

### Example 15: Preparation of methyl 2-methyl-4-(1-(4-(trifluoromethyl)phenyl)-1H-1,2,4-triazol-3-yl)benzoate (C19)

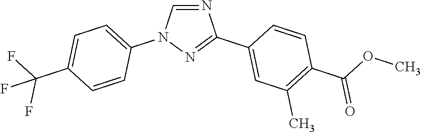 [SMILES: COC(=O)c1ccc(-c2ncn(-c3ccc(C(F)(F)F)cc3)n2)cc1C]

To three separate microwave vials were added 3-bromo-1-(4-(trifluoromethyl)phenyl)-1H-1,2,4-triazole (prepared as in Fischer at al., United States Patent Application Publication 20140274688 A1; 1 g, 3.25 mmol), methyl 2-methyl-4-(4,4,5,5-tetramethyl-1,3,2-dioxaborolan-2-yl)benzoate (0.896 g, 3.25 mmol), Pd(PPh₃)₄ (0.375 g, 0.33 mmol), and sodium bicarbonate (0.818 g, 9.74 mmol). The reagents were dissolved in dioxane (12 mL) and water (3 mL). The vials were heated in a Biotage® microwave reactor at 140° C. for 30 min. The reactions were combined and diluted with EtOAc and washed with water. The organic layers were combined, dried, filtered, and concentrated. Purification using 0-10% EtOAc/(1:1 hexanes:DCM) as eluent provided the title compound as a white solid (1.48 g, 36%): ¹H NMR (400 MHz, CDCl₃) δ 8.68 (s, 1H), 8.15-8.05 (m, 2H), 8.03 (d, J=8.1 Hz, 1H), 7.93 (dd, J=9.0, 0.9 Hz, 2H), 7.81 (dd, J=8.9, 0.9 Hz, 2H), 3.93 (s, 3H), 2.70 (s, 3H); ¹⁹F NMR (376 MHz, CDCl₃) δ −62.52; ESIMS m/z 362 ([M+H]⁺).

The following compounds were prepared in accordance to the procedure in Example 15.

### Methyl 2-methyl-4-(1-(4-(perfluoroethoxy)phenyl)-1H-1,2,4-triazol-3-yl)benzoate (C20)

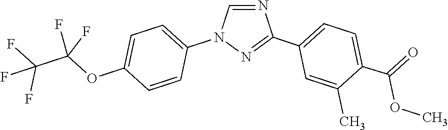 [SMILES: COC(=O)c1ccc(-c2ncn(-c3ccc(OC(F)(F)C(F)(F)F)cc3)n2)cc1C]

The title compound was prepared and was isolated as a white solid (527 mg, 43%): ¹H NMR (400 MHz, CDCl₃) δ 8.60 (s, 1H), 8.17-7.98 (m, 3H), 7.82 (d, J=9.0 Hz, 2H), 7.41 (d, J=8.8 Hz, 2H), 3.92 (s, 3H), 2.70 (s, 3H); ¹⁹F NMR (376 MHz, CDCl₃) δ −85.90, −87.86.

### Methyl 2-methoxy-4-(1-(4-(perfluoroethoxy)phenyl)-1H-1,2,4-triazol-3-yl)benzoate (C21)

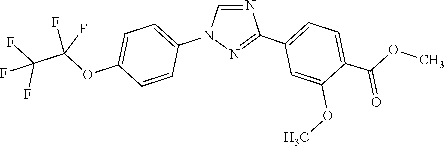 [SMILES: COC(=O)c1ccc(-c2ncn(-c3ccc(OC(F)(F)C(F)(F)F)cc3)n2)cc1OC]

The title compound was prepared and was isolated as a white solid (517 mg, 44%): ¹H NMR (400 MHz, CDCl₃) δ 8.60 (s, 1H), 7.92 (d, J=8.4 Hz, 1H), 7.86-7.78 (m, 4H), 7.41 (d, J=8.8 Hz, 2H), 4.04 (s, 3H), 3.92 (s, 3H); ¹⁹F NMR (376 MHz, CDCl₃) δ −85.89, −87.86.

### Methyl 3-methyl-4-(1-(4-(perfluoroethoxy)phenyl)-1H-1,2,4-triazol-3-yl)benzoate (C22)

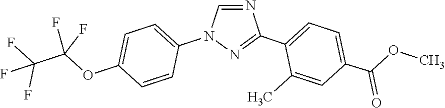 [SMILES: COC(=O)c1ccc(-c2ncn(-c3ccc(OC(F)(F)C(F)(F)F)cc3)n2)c(C)c1]

The title compound was prepared and was isolated as a white solid (537 mg, 44%): ¹H NMR (400 MHz, CDCl₃) δ 8.63 (s, 1H), 8.17 (d, J=8.1 Hz, 1H), 8.01 (dd, J=1.3, 0.6 Hz, 1H), 7.96 (ddd, J=8.1, 1.8, 0.7 Hz, 1H), 7.82 (d, J=9.0 Hz, 2H), 7.46-7.35 (m, 2H), 3.95 (s, 3H), 2.77 (s, 3H); ¹⁹F NMR (376 MHz, CDCl₃) δ −85.89, 15-87.86.

### Methyl 3-methyl-4-(1-(4-(trifluoromethoxy)phenyl)-1H-1,2,4-triazol-3-yl)benzoate (C23)

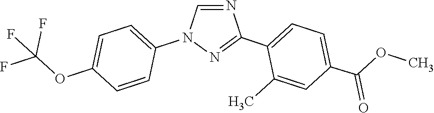 [SMILES: COC(=O)c1ccc(-c2ncn(-c3ccc(OC(F)(F)F)cc3)n2)c(C)c1]

The title compound was prepared and was isolated as a white solid (1.45 g, 47%): ¹H NMR (400 MHz, CDCl₃) δ 8.63 (s, 1H), 8.02-7.93 (m, 2H), 7.81 (d, J=9.0 Hz, 2H), 7.73-7.63 (m, 2H), 7.40 (dd, J=9.0, 1.0 Hz, 1H), 3.95 (s, 3H), 2.77 (s, 3H); ESIMS m/z ([M]⁺) 377.

### Methyl 2-methoxy-4-(1-(4-(trifluoromethoxy)phenyl)-1H-1,2,4-triazol-3-yl)benzoate (C24)

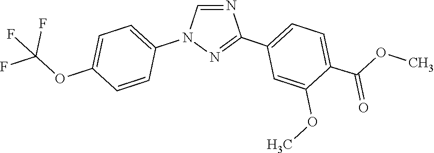 [SMILES: COC(=O)c1ccc(-c2ncn(-c3ccc(OC(F)(F)F)cc3)n2)cc1OC]

The title compound was prepared and was isolated as a white solid (1.67 g, 100%): ¹H NMR (400 MHz, CDCl₃) δ 8.59 (s, 1H), 7.84-7.79 (m, 2H), 7.73-7.62 (m, 2H), 7.43-7.38 (m, 2H), 7.35-7.30 (m, 1H), 4.03 (s, 3H), 3.92 (s, 3H); ESIMS m/z ([M]⁺) 393.

### Methyl 2-fluoro-4-(1-(4-(trifluoromethyl)phenyl)-1H-1,2,4-triazol-3-yl)benzoate (C25)

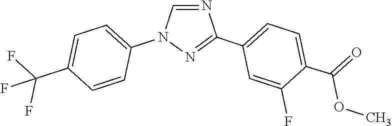 [SMILES: COC(=O)c1ccc(-c2ncn(-c3ccc(C(F)(F)F)cc3)n2)cc1F]

The title compound was prepared and was isolated as a white solid (553 mg, 79%): ¹H NMR (400 MHz, CDCl₃) δ 8.69 (s, 1H), 8.08-8.04 (m, 2H), 8.03-7.97 (m, 1H), 7.92 (dd, J=9.1, 0.8 Hz, 2H), 7.82 (dd, J=8.9, 0.8 Hz, 2H), 3.97 (s, 3H); ESIMS m/z ([M]⁺) 365.

### Methyl 2-methoxy-4-(1-(4-(trifluoromethyl)phenyl)-1H-1,2,4-triazol-3-yl)benzoate (C26)

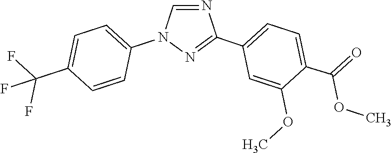 [SMILES: COC(=O)c1ccc(-c2ncn(-c3ccc(C(F)(F)F)cc3)n2)cc1OC]

The title compound was prepared and was isolated as a white solid (1.40 g, 84%): ¹H NMR (400 MHz, CDCl₃) δ 8.68 (s, 1H), 8.00-7.89 (m, 2H), 7.89-7.76 (m, 4H), 7.72-7.61 (m, 1H), 4.04 (s, 3H), 3.93 (s, 3H); ESIMS m/z ([M]⁺) 377.

### Methyl 3-methyl-4-(1-(4-(trifluoromethyl)phenyl)-1H-1,2,4-triazol-3-yl)benzoate (C27)

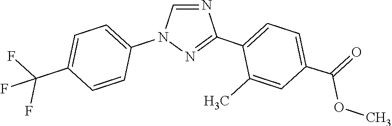 [SMILES: COC(=O)c1ccc(-c2ncn(-c3ccc(C(F)(F)F)cc3)n2)c(C)c1]

The title compound was prepared and was isolated as a white solid (449 mg, 37%): ¹H NMR (400 MHz, CDCl₃) δ 8.71 (s, 1H), 8.19 (d, J=8.1 Hz, 1H), 8.01 (dd, J=1.2, 0.6 Hz, 1H), 7.97 (ddd, J=8.1, 1.8, 0.7 Hz, 1H), 7.95-7.88 (m, 2H), 7.84-7.78 (m, 2H), 3.95 (s, 3H), 2.78 (s, 3H); ¹⁹F NMR (376 MHz, CD₃OD) δ −64.03.

### Example 16: Preparation of 2-methyl-4-(1-(4-(trifluoromethyl)phenyl)-1H-1,2,4-triazol-3-yl)benzoic acid (C28)

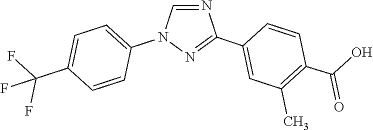 [SMILES: Cc1cc(-c2ncn(-c3ccc(C(F)(F)F)cc3)n2)ccc1C(=O)O]

A solution of methyl 2-methyl-4-(1-(4-(trifluoromethyl)phenyl)-1H-1,2,4-triazol-3-yl)benzoate (C19; 1.48 g, 3.93 mmol) in THF (40 mL) was added to a 250 mL round-bottomed flask. Methanol (40 mL) and 2 N sodium hydroxide (20 mL, 40 mmol) were added sequentially. The reaction mixture was stirred at room temperature for 4 h. The solution was made acidic with 1 N HCl and was diluted in EtOAc and washed with water. The organic layer was dried, filtered, and concentrated. The title compound was isolated as a white solid (1.71 g, 96%): ¹H NMR (400 MHz, CD₃OD) δ 9.27 (s, 1H), 8.14 (dd, J=8.7, 1.0 Hz, 2H), 8.11-8.05 (m, 1H), 8.03 (dd, J=2.2, 1.1 Hz, 2H), 7.92-7.85 (m, 2H), 2.67 (s, 3H); ¹⁹F NMR (376 MHz, CD₃OD) δ −64.01; ESIMS m/z 348 ([M+H]⁺).

The following compounds were prepared in accordance to the procedure in Example 16.

### 2-Methyl-4-(1-(4-(perfluoroethoxy)phenyl)-1H-1,2,4-triazol-3-yl)benzoic acid (C29)

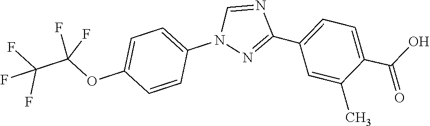 [SMILES: Cc1cc(-c2ncn(-c3ccc(OC(F)(F)C(F)(F)F)cc3)n2)ccc1C(=O)O]

The title compound was prepared and was isolated as a white solid (466 mg, 84%): ¹H NMR (400 MHz, CD₃OD) δ 9.18 (s, 1H), 8.14-7.96 (m, 5H), 7.51 (d, J=8.8 Hz, 2H), 2.67 (s, 3H), 1.35 (s, 1H); ¹⁹F NMR (376 MHz, CD₃OD) δ −87.67, −89.08.

### 2-Methoxy-4-(1-(4-(perfluoroethoxy)phenyl)-1H-1,2,4-triazol-3-yl)benzoic acid (C30)

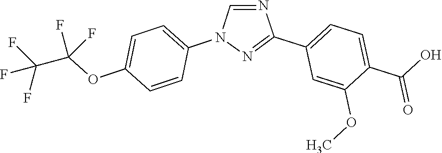 [SMILES: COc1cc(-c2ncn(-c3ccc(OC(F)(F)C(F)(F)F)cc3)n2)ccc1C(=O)O]

The title compound was prepared and was isolated as a white solid (634 mg, 77%): ¹H NMR (400 MHz, CD₃OD) δ 9.20 (s, 1H), 8.05 (d, J=9.0 Hz, 2H), 7.95-7.86 (m, 2H), 7.81 (dd, J=8.0, 1.4 Hz, 1H), 7.51 (d, J=8.9 Hz, 2H), 4.01 (s, 3H); ¹⁹F NMR (376 MHz, CD₃OD) δ −87.67, −89.08.

### 3-Methyl-4-(1-(4-(perfluoroethoxy)phenyl)-1H-1,2,4-triazol-3-yl)benzoic acid (C31)

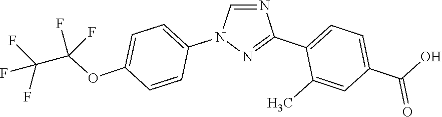 [SMILES: Cc1cc(C(=O)O)ccc1-c1ncn(-c2ccc(OC(F)(F)C(F)(F)F)cc2)n1]

The title compound was prepared and was isolated as a white solid (949 mg, 21%): ¹H NMR (400 MHz, CD₃OD) δ 9.21 (s, 1H), 8.09 (d, J=8.1 Hz, 1H), 8.05 (d, J=9.0 Hz, 2H), 8.01 (dt, J=1.5, 0.7 Hz, 1H), 7.95 (ddd, J=8.1, 1.8, 0.7 Hz, 1H), 7.52 (d, J=8.9 Hz, 2H), 2.75 (s, 3H); ¹⁹F NMR (376 MHz, CD₃OD) δ −87.68, −89.09.

### 3-Methyl-4-(1-(4-(trifluoromethoxy)phenyl)-1H-1,2,4-triazol-3-yl)benzoic acid (C32)

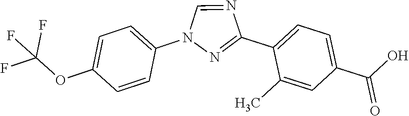 [SMILES: Cc1cc(C(=O)O)ccc1-c1ncn(-c2ccc(OC(F)(F)F)cc2)n1]

The title compound was prepared and was isolated as a white solid (394 mg, 45%): ¹H NMR (400 MHz, CD₃OD) δ 9.19 (s, 1H), 8.08 (d, J=8.1 Hz, 1H), 8.05-7.99 (m, 2H), 7.68-7.61 (m, 2H), 7.58-7.48 (m, 2H), 2.74 (s, 3H), 2.15 (s, 1H); ¹⁹F NMR (376 MHz, CDCl₃) δ −58.04.

### 2-Methoxy-4-(1-(4-(trifluoromethoxy)phenyl)-1H-1,2,4-triazol-3-yl)benzoic acid (C33)

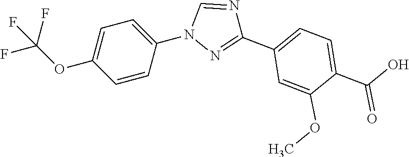 [SMILES: COc1cc(-c2ncn(-c3ccc(OC(F)(F)F)cc3)n2)ccc1C(=O)O]

The title compound was prepared and was isolated as a white solid (719 mg, 86%): ¹H NMR (400 MHz, CDCl₃) δ 8.61 (s, 1H), 8.30 (d, J=8.2 Hz, 1H), 7.82 (d, J=9.0 Hz, 1H), 7.71-7.63 (m, 2H), 7.58-7.52 (m, 1H), 7.51-7.44 (m, 2H), 4.21 (s, 3H), 1.57 (s, 1H); ESIMS m/z ([M]⁺) 379.

### 2-Fluoro-4-(1-(4-(trifluoromethyl)phenyl)-1H-1,2,4-triazol-3-yl)benzoic acid (C34)

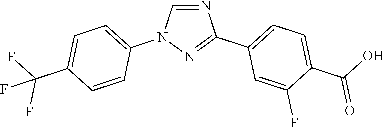 [SMILES: O=C(O)c1ccc(-c2ncn(-c3ccc(C(F)(F)F)cc3)n2)cc1F]

The title compound was prepared and was isolated as a white solid (459 mg, 86%): ¹H NMR (400 MHz, CD₃OD) δ 9.30 (s, 1H), 8.16 (d, J=8.4 Hz, 2H), 8.06 (d, J=3.5 Hz, 2H), 7.96 (d, J=11.7 Hz, 1H), 7.90 (d, J=8.5 Hz, 2H), 2.15 (s, 1H); ¹⁹F NMR (376 MHz, CD₃OD) δ −64.05, −111.19.

### 3-Methyl-4-(1-(4-(trifluoromethyl)phenyl)-1H-1,2,4-triazol-3-yl)benzoic acid (C35)

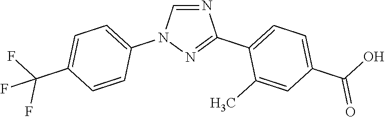 [SMILES: Cc1cc(C(=O)O)ccc1-c1ncn(-c2ccc(C(F)(F)F)cc2)n1]

The title compound was prepared and was isolated as a white solid (430 mg, 100%): ¹H NMR (400 MHz, CD₃OD) δ 9.31 (s, 1H), 8.18-8.09 (m, 3H), 8.01 (dd, J=1.2, 0.6 Hz, 1H), 7.95 (ddd, J=8.0, 1.8, 0.7 Hz, 1H), 7.92-7.88 (m, 2H), 2.76 (s, 3H); ¹⁹F NMR (376 MHz, CD₃OD) δ −64.02.

### Example 17: Preparation of 2-methyl-4-(1-(4-(trifluoromethyl)phenyl)-1H-1,2,4-triazol-3-yl)benzoyl azide (C36)

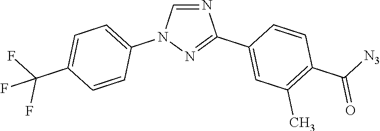 [SMILES: Cc1cc(-c2ncn(-c3ccc(C(F)(F)F)cc3)n2)ccc1C(=O)N=[N+]=[N-]]

2-Methyl-4-(1-(4-(trifluoromethyl)phenyl)-1H-1,2,4-triazol-3-yl)benzoic acid (C28; 1.7 g, 4.68 mmol) was partially dissolved in isopropyl alcohol (50 mL) in a 100 mL round-bottomed flask. Triethylamine (1 mL, 7.02 mmol) and diphenylphosphoryl azide (1.4 mL, 6.55 mmol) were added sequentially. The reaction mixture was stirred at room temperature for 20 h and poured into ice water. The white precipitate was collected by vacuum filtration. The precipitate was washed with isopropyl alcohol and hexanes and was dried. The title compound was isolated as a white solid (596 mg, 30%): ¹H NMR (400 MHz, CDCl₃) δ 8.69 (s, 1H), 8.17-8.13 (m, 1H), 8.10-8.02 (m, 2H), 7.96-7.90 (m, 2H), 7.82 (d, J=8.7 Hz, 2H), 2.75 (s, 3H); ¹⁹F NMR (376 MHz, CDCl₃) δ −62.53; ESIMS m/z 345 ([M+H]⁺) (isocyanate).

The following compounds were prepared in accordance to the procedure in Example 17.

### 2-Fluoro-4-(1-(4-(trifluoromethyl)phenyl)-1H-1,2,4-triazol-3-yl)benzoyl azide (C37)

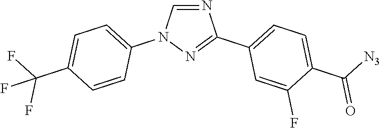 [SMILES: [N-]=[N+]=NC(=O)c1ccc(-c2ncn(-c3ccc(C(F)(F)F)cc3)n2)cc1F]

The title compound was prepared and was isolated as a white solid (299 mg, 60%): ¹H NMR (400 MHz, CDCl₃) δ 8.70 (s, 1H), 8.10-7.98 (m, 3H), 7.92 (d, J=8.9 Hz, 2H), 7.87-7.78 (m, 2H); ¹⁹F NMR (376 MHz, CDCl₃) δ−62.58, −107.88.

### 2-Methyl-4-(1-(4-(perfluoroethoxy)phenyl)-1H-1,2,4-triazol-3-yl)benzoyl azide (C38)

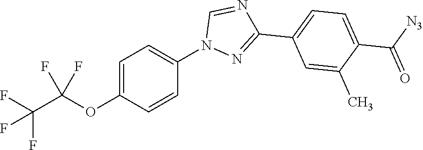 [SMILES: Cc1cc(-c2ncn(-c3ccc(OC(F)(F)C(F)(F)F)cc3)n2)ccc1C(=O)N=[N+]=[N-]]

The title compound was prepared and was isolated as a beige solid (208 mg, 40%): ¹H NMR (400 MHz, CDCl₃) δ 8.61 (s, 1H), 8.14-8.11 (m, 1H), 8.08-8.04 (m, 2H), 7.82 (d, J=9.0 Hz, 2H), 7.41 (d, J=8.9 Hz, 2H), 2.74 (s, 3H); ¹⁹F NMR (376 MHz, CDCl₃) δ−85.89, −87.86.

### 2-Methoxy-4-(1-(4-(perfluoroethoxy)phenyl)-1H-1,2,4-triazol-3-yl)benzoyl azide (C39)

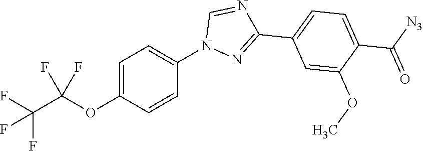 [SMILES: COc1cc(-c2ncn(-c3ccc(OC(F)(F)C(F)(F)F)cc3)n2)ccc1C(=O)N=[N+]=[N-]]

The title compound was prepared from methyl 2-methoxy-4-(1-(4-(perfluoroethoxy)phenyl)-1H-1,2,4-triazol-3-yl)benzoate (C21) over two steps via the acid and was isolated as a white solid (162 mg, 18%): ¹H NMR (400 MHz, CDCl₃) δ 8.61 (s, 1H), 7.96 (d, J=8.7 Hz, 1H), 7.86-7.78 (m, 4H), 7.42 (d, J=8.8 Hz, 2H), 4.06 (s, 3H); ¹⁹F NMR (376 MHz, CDCl₃) δ −85.88, −87.86.

### 3-Methyl-4-(1-(4-(perfluoroethoxy)phenyl)-1H-1,2,4-triazol-3-yl)benzoyl azide (C40)

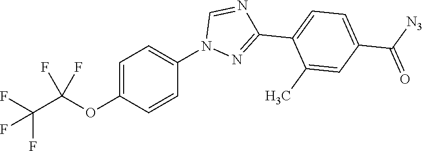 [SMILES: Cc1cc(C(=O)N=[N+]=[N-])ccc1-c1ncn(-c2ccc(OC(F)(F)C(F)(F)F)cc2)n1]

The title compound was prepared and was isolated as a white solid (547 mg, 54%): ¹H NMR (400 MHz, CDCl₃) δ 8.64 (s, 1H), 8.21 (d, J=8.1 Hz, 1H), 7.99 (dd, J=1.3, 0.6 Hz, 1H), 7.95 (ddd, J=8.2, 1.9, 0.7 Hz, 1H), 7.82 (d, J=9.0 Hz, 2H), 7.46-7.35 (m, 2H), 2.78 (s, 3H); ¹⁹F NMR (376 MHz, CDCl₃) δ−85.89, −87.87.

### 3-Methyl-4-(1-(4-(trifluoromethoxy)phenyl)-1H-1,2,4-triazol-3-yl)benzoyl azide (C41)

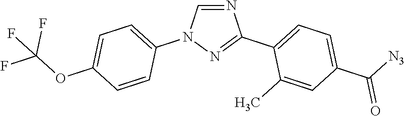 [SMILES: Cc1cc(C(=O)N=[N+]=[N-])ccc1-c1ncn(-c2ccc(OC(F)(F)F)cc2)n1]

The title compound was prepared and was isolated as a white solid (312 mg, 72%): ¹H NMR (400 MHz, CDCl₃) δ 8.63 (s, 1H), 8.20 (d, J=8.2 Hz, 1H), 8.06-7.89 (m, 2H), 7.81 (d, J=9.0 Hz, 2H), 7.41 (dd, J=9.1, 0.9 Hz, 2H), 2.78 (s, 3H); ESIMS m/z ([M-27]⁺) 361.

### 2-Methoxy-4-(1-(4-(trifluoromethoxy)phenyl)-1H-1,2,4-triazol-3-yl)benzoyl azide (C₄₂)

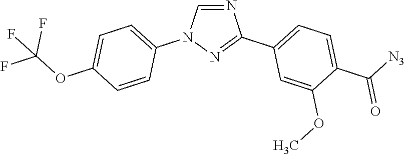 [SMILES: COc1cc(-c2ncn(-c3ccc(OC(F)(F)F)cc3)n2)ccc1C(=O)N=[N+]=[N-]]

The title compound was prepared and was isolated as a white solid (331 mg, 40%): ¹H NMR (400 MHz, CDCl₃) δ 8.60 (s, 1H), 7.96 (d, J=8.7 Hz, 1H), 7.88-7.75 (m, 4H), 7.41 (dd, J=9.1, 0.9 Hz, 2H), 4.06 (s, 3H); ESIMS m/z ([M-27]⁺) 377.

### 2-Methoxy-4-(1-(4-(trifluoromethyl)phenyl)-1H-1,2,4-triazol-3-yl)benzoyl azide (C43)

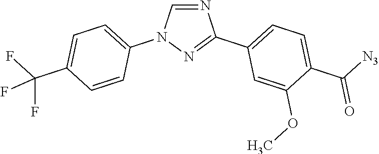 [SMILES: COc1cc(-c2ncn(-c3ccc(C(F)(F)F)cc3)n2)ccc1C(=O)N=[N+]=[N-]]

The title compound was prepared and was isolated as a beige solid (793 mg, 33%): ¹H NMR (400 MHz, CDCl₃) δ 8.69 (s, 1H), 7.95 (dd, J=15.9, 8.5 Hz, 3H), 7.87-7.80 (m, 4H), 4.06 (s, 3H); ¹⁹F NMR (376 MHz, CDCl₃) δ−62.56.

### 3-Methyl-4-(1-(4-(trifluoromethyl)phenyl)-1H-1,2,4-triazol-3-yl)benzoyl azide (C44)

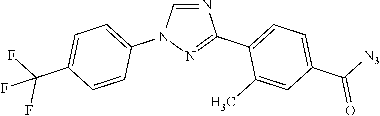 [SMILES: Cc1cc(C(=O)N=[N+]=[N-])ccc1-c1ncn(-c2ccc(C(F)(F)F)cc2)n1]

The title compound was prepared and was isolated as a white solid (387 mg, 81%): ¹H NMR (400 MHz, CDCl₃) δ 8.72 (s, 1H), 8.22 (d, J=8.1 Hz, 1H), 8.03-7.89 (m, 4H), 7.82 (dd, J=9.0, 0.9 Hz, 2H), 2.79 (s, 3H); ¹⁹F NMR (376 MHz, CDCl₃) δ−62.55.

### Example 18: Preparation of 3-bromo-1-(3-(trifluoromethyl)phenyl)-1H-1,2,4-triazole (C45)

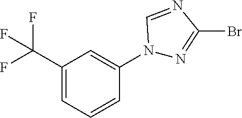 [SMILES: FC(F)(F)c1cccc(-n2cnc(Br)n2)c1]

Copper(I) iodide (0.84 g, 4.41 mmol) and cesium carbonate (29 g, 88.2 mmol) were added sequentially to a solution of 3-bromo-1H-1,2,4-triazole (13.05 g, 88.2 mmol) and 3-trifluoromethylphenyl iodide (12 g, 44.1 mmol) in dimethyl sulfoxide (DMSO; 120 mL). The reaction mixture was stirred at 100° C. for 36 h. The reaction mixture was cooled, diluted with EtOAc (300 mL), and filtered through a pad of diatomaceous earth, which was washed with EtOAc. The filtrate was washed with ice water (2×200 mL) and brine (100 mL). The organic layer was dried over anhydrous Na₂SO₄, filtered, and concentrated under reduced pressure. Purification by column chromatography (silica gel 100-200 mesh; 15-25% EtOAc-petroleum ether) provided the title compound as a pale yellow liquid (9 g, 80%): ¹H NMR (400 MHz, CDCl₃) δ 8.52 (s, 1H), 7.96 (s, 1H), 7.87-7.85 (m, 1H), 7.72-7.67 (m, 2H).

The following compound was prepared in accordance to the procedure in Example 18.

### 3-Bromo-1-(3-(trifluoromethoxy)phenyl)-1H-1,2,4-triazole (C46)

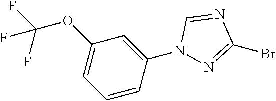 [SMILES: FC(F)(F)Oc1cccc(-n2cnc(Br)n2)c1]

The title compound was prepared from 3-trifluoromethoxyphenyl iodide and was isolated as a brown liquid (9.5 g, 74%): ¹H NMR (400 MHz, CDCl₃) δ 8.47 (s, 1H), 7.65-7.35 (m, 3H), 7.30-7.28 (m, 1H).

### 3-Bromo-1-(p-tolyl)-1H-1,2,4-triazole (C47)

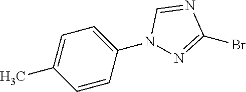

The title compound was prepared from p-tolyl iodide and was isolated as a pale yellow solid (1.5 g, 27%): ESIMS m/z 238 ([M+H]⁺).

### 3-Bromo-1-(4-methoxyphenyl)-1H-1,2,4-triazole (C48)

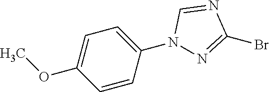

The title compound was prepared from 4-methoxyphenyl iodide and was isolated as a pale yellow solid (1 g, 21%): ¹H NMR (400 MHz, CDCl₃) δ 8.31 (s, 1H), 7.54-7.51 (m, 2H), 7.02-6.99 (m, 2H), 3.86 (s, 3H).

### Example 19. Preparation of 2-fluoro-4-(1-(3-(trifluoromethyl)phenyl)-1H-1,2,4-triazol-3-yl)aniline (C49)

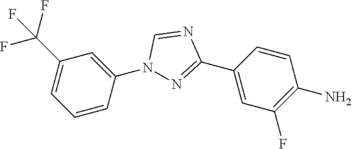 [SMILES: Nc1ccc(-c2ncn(-c3cccc(C(F)(F)F)c3)n2)cc1F]

To an argon-degassed solution of 3-bromo-1-(3-(trifluoromethyl)phenyl)-1H-1,2,4-triazole (C46; 1.62 g, 5.6 mmol) in dioxane-water (3:1 ratio; 10 mL) were added sequentially 2-fluoro-4-(4,4,5,5-tetramethyl-1,3,2-dioxaborolan-2-yl)aniline (1.5 g, 6.2 mmol) and potassium carbonate (1.16 g, 8.4 mmol), Pd(PPh₃)₄ (0.65 g, 0.56 mmol). The reaction mixture was stirred at 110° C. for 16 h. The reaction mixture was cooled, diluted with EtOAc (20 mL) and was washed with water. The aqueous layer was extracted with EtOAc (2×10 mL). The organic layers were washed with brine, dried over anhydrous Na₂SO₄, filtered, and concentrated under reduced pressure. Purification of the reaction product by column chromatography (silica gel 100-200 mesh; 30-50% EtOAc-petroleum ether) provided the title compound as a pale yellow solid (1.8 g, 61%): mp 125-137° C.; ¹H NMR (400 MHz, DMSO-d₆) δ 9.43 (s, 1H), 8.28 (s, 1H), 8.24 (d, J=8.0 Hz, 1H), 7.84-7.75 (m, 2H), 7.67 (s, 1H), 7.64 (d, J=5.6 Hz, 1H), 6.88-6.83 (m, 1H), 5.59 (br s, 2H); ¹³C NMR (101 MHz, DMSO-d₆) δ 161.97, 151.34, 149.01, 143.58, 138.19, 137.33, 131.17, 130.71, 130.15, 123.82, 122.84, 117.80, 115.92, 115.46, 112.71; ¹⁹F NMR (376 MHz, DMSO-d₆) δ −61.23, −135.10; ESIMS m/z 323 ([M+H]⁺).

The following compounds were prepared in accordance to the procedure in Example 19.

### 2-Methyl-4-(1-(3-(trifluoromethyl)phenyl)-1H-1,2,4-triazol-3-yl)aniline (C50)

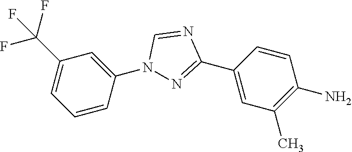 [SMILES: Cc1cc(-c2ncn(-c3cccc(C(F)(F)F)c3)n2)ccc1N]

The title compound was prepared and was isolated as a yellow solid (1.5 g, 53%): mp 135-140° C.; ¹H NMR (400 MHz, DMSO-d₆) δ 9.40 (s, 1H), 8.28 (s, 1H), 8.25 (d, J=8.0 Hz, 1H), 7.83-7.81 (m, 4H), 6.70 (d, J=8.4 Hz, 1H), 5.27 (br s, 2H) 2.14 (s, 3H); ¹³C NMR (101 MHz, DMSO-d₆) δ 163.02, 148.38, 143.25, 137.42, 131.07, 130.41, 128.13, 125.07, 123.55, 122.62, 122.36, 120.78, 117.63, 115.27, 113.62, 17.44; ¹⁹F NMR (376 MHz, DMSO-d₆) −61.20; ESIMS m/z 319 ([M+H]⁺).

### 2-Fluoro-4-(1-(3-(trifluoromethoxy)phenyl)-1H-1,2,4-triazol-3-yl)aniline (C51)

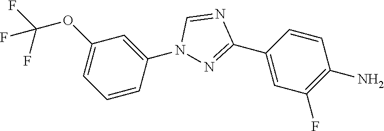 [SMILES: Nc1ccc(-c2ncn(-c3cccc(OC(F)(F)F)c3)n2)cc1F]

The title compound was prepared and was isolated as a pale yellow solid (1.7 g, 55%): mp 142-144° C.; ¹H NMR (300 MHz, DMSO-d₆) δ 9.37 (s, 1H), 7.99 (s, 1H), 7.96 (s, 1H), 7.73-7.62 (m, 3H), 7.41 (d, J=8.4 Hz, 1H), 6.86 (t, J=8.8 Hz, 1H), 5.59 (br s, 2H); ¹³C NMR (75 MHz, DMSO-d₆) δ 161.95, 151.76, 148.99, 148.62, 143.53, 138.30, 138.09, 131.73, 122.94, 119.48, 117.77, 115.92, 112.83, 112.56, 111.85; ¹⁹F NMR (282 MHz, DMSO-d₆) δ−56.81; ESIMS m/z 339 ([M+H]⁺).

### 2-Methyl-4-(1-(3-(trifluoromethoxy)phenyl)-1H-1,2,4-triazol-3-yl)aniline (C52)

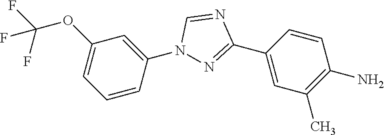 [SMILES: Cc1cc(-c2ncn(-c3cccc(OC(F)(F)F)c3)n2)ccc1N]

The title compound was prepared and was isolated as a white solid (1 g, 31%): mp 141-146° C.; ¹H NMR (400 MHz, DMSO-d₆) δ 9.34 (s, 1H), 7.99-7.95 (m, 2H), 7.72-7.66 (m, 3H), 7.42-7.38 (m, 1H), 6.69 (d, J=8.0 Hz, 1H), 5.27 (s, 2H), 2.14 (s, 3H); ¹⁹F NMR (376 MHz, DMSO-d₆) δ−56.78; ESIMS m/z 335 ([M+H]⁺).

### 2-Fluoro-4-(1-(p-tolyl)-1H-1,2,4-triazol-3-yl)aniline (C₅₃)

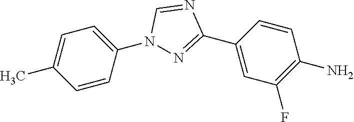 [SMILES: Cc1ccc(-n2cnc(-c3ccc(N)c(F)c3)n2)cc1]

The title compound was prepared and was isolated as a pale yellow solid (2.2 g, 77%): mp 159-163° C.; ¹H NMR (400 MHz, DMSO-d₆) δ 9.18 (s, 1H), 7.75 (d, J=8.4 Hz, 2H), 7.61 (d, J=1.2 Hz, 1H), 7.60 (dd, J=1.6, 8.4 Hz, 1H), 7.33 (d, J=8.0 Hz, 2H), 6.82 (t, J=8.8 Hz, 1H), 5.51 (br s, 2H), 2.33 (s, 3H); ¹³C NMR (101 MHz, DMSO-d₆) δ 161.52, 151.40, 149.04, 142.71, 137.87, 136.95, 134.59, 130.08, 122.71, 118.36, 115.96, 112.61, 20.53; ¹⁹F NMR (376 MHz, DMSO-d₆) δ −135.12; ESIMS m/z 269 ([M+H]⁺).

### 2-Fluoro-4-(1-(4-methoxyphenyl)-1H-1,2,4-triazol-3-yl)aniline (C54)

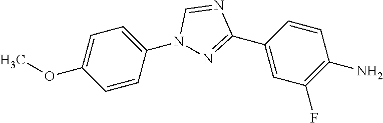 [SMILES: COc1ccc(-n2cnc(-c3ccc(N)c(F)c3)n2)cc1]

The title compound was prepared and was isolated as a pale yellow solid (1.0 g, 63%): mp 119-124° C.; ¹H NMR (400 MHz, DMSO-d₆) δ 9.12 (s, 1H), 7.80 (d, J=9.2 Hz, 2H), 7.68-7.57 (m, 2H), 7.11 (d, J=9.2 Hz, 2H), 6.87-6.80 (m, 1H), 5.50 (s, 2H), 3.83 (s, 3H); ¹³C NMR (101 MHz, DMSO-d₆) δ 161.38, 158.42, 149.01, 142.54, 137.72, 130.28, 122.57, 120.75, 118.45, 115.90, 114.70, 112.42, 55.46; ¹⁹F NMR (376 MHz, DMSO-d₆) δ−135.17; ESIMS m/z 285 ([M+H]⁺).

### Example 20: Preparation of 2-fluoro-4-(1-(4-(trifluoromethoxy)phenyl)-1H-1,2,4-triazol-3-yl)aniline (C55)

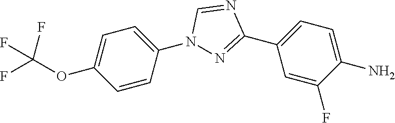 [SMILES: Nc1ccc(-c2ncn(-c3ccc(OC(F)(F)F)cc3)n2)cc1F]

To two separate 10-20 mL microwave vials were added 3-bromo-1-(4-(trifluoromethoxy)phenyl)-1H-1,2,4-triazole (prepared as in Fischer at al., United States Patent Application Publication 20140274688 A1; 0.5 g, 1.623 mmol), 2-fluoro-4-(4,4,5,5-tetramethyl-1,3,2-dioxaborolan-2-yl)aniline (0.385 g, 1.623 mmol), Pd(PPh₃)₄ (0.188 g, 0.162 mmol), and sodium bicarbonate (0.409 g, 4.87 mmol). The solids were dissolved in dioxane (6.49 mL) and water (1.623 mL). The vials were placed in a Biotage® microwave reactor and were heated at 140° C. for 30 min each. The solutions were cooled to room temperature, diluted with EtOAc, and washed with water. The organic layers were dried, filtered, and concentrated. Purification by flash chromatography (silica; EtOAc-hexanes) yielded the title compound as a gummy yellow semi-solid (718 mg, 62%): ¹H NMR (400 MHz, CDCl₃) δ 8.51 (s, 1H), 7.88-7.71 (m, 4H), 7.41-7.33 (m, 2H), 6.85 (dd, J=9.0, 8.2 Hz, 1H), 3.93 (s, 2H); ¹⁹F NMR (376 MHz, CDCl₃) δ−58.04, −135.37; ESIMS m/z 339 ([M+H]⁺).

The following compounds were prepared in accordance to the procedure in Example 20.

### 2-Methyl-4-(1-(4-(trifluoromethoxy)phenyl)-1H-1,2,4-triazol-3-yl)aniline (C56)

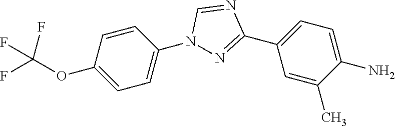 [SMILES: Cc1cc(-c2ncn(-c3ccc(OC(F)(F)F)cc3)n2)ccc1N]

The title compound was prepared and was isolated as a yellow solid (700 mg, 63%): ¹H NMR (400 MHz, CDCl₃) δ 8.50 (d, J=1.3 Hz, 1H), 7.97-7.83 (m, 2H), 7.83-7.70 (m, 2H), 7.36 (d, J=8.6 Hz, 2H), 6.75 (d, J=8.2 Hz, 1H), 3.81 (s, 2H), 2.24 (s, 3H); ¹⁹F NMR (376 MHz, CDCl₃) δ−58.04; ESIMS m/z 335 ([M+H]⁺).

### 2-Chloro-4-(1-(4-(trifluoromethoxy)phenyl)-1H-1,2,4-triazol-3-yl)aniline (C57)

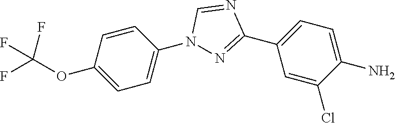 [SMILES: Nc1ccc(-c2ncn(-c3ccc(OC(F)(F)F)cc3)n2)cc1Cl]

The title compound was prepared and was isolated as a yellow solid (859 mg, 73%): ¹H NMR (400 MHz, CDCl₃) δ 8.51 (s, 1H), 8.11 (d, J=1.9 Hz, 1H), 7.89 (dd, J=8.3, 1.9 Hz, 1H), 7.84-7.69 (m, 2H), 7.46-7.32 (m, 2H), 6.84 (d, J=8.4 Hz, 1H), 4.26 (s, 2H); ¹⁹F NMR (376 MHz, CDCl₃) δ −58.03; ESIMS m/z 355 ([M+H]⁺).

### 2-Ethyl-4-(1-(4-(trifluoromethoxy)phenyl)-1H-1,2,4-triazol-3-yl)aniline (C58)

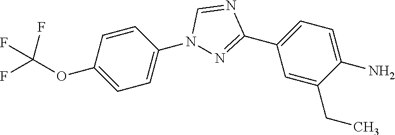 [SMILES: CCc1cc(-c2ncn(-c3ccc(OC(F)(F)F)cc3)n2)ccc1N]

The title compound was prepared and was isolated as an off-white solid (500 mg, 43%): ¹H NMR (400 MHz, CDCl₃) δ 8.51 (s, 1H), 7.97-7.83 (m, 2H), 7.83-7.73 (m, 2H), 7.37 (dq, J=8.0, 1.0 Hz, 2H), 6.76 (d, J=8.2 Hz, 1H), 3.84 (s, 2H), 2.59 (q, J=7.5 Hz, 2H), 1.33 (t, J=7.5 Hz, 3H); ¹⁹F NMR (376 MHz, CDCl₃) δ −58.04; ESIMS m/z 349 ([M+H]⁺).

### 2-Methoxy-4-(1-(4-(trifluoromethoxy)phenyl)-1H-1,2,4-triazol-3-yl)aniline (C59)

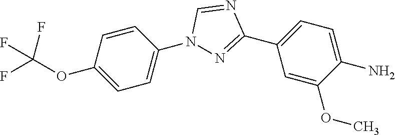 [SMILES: COc1cc(-c2ncn(-c3ccc(OC(F)(F)F)cc3)n2)ccc1N]

The title compound was prepared and was isolated as an off-white solid (678 mg, 58%): ¹H NMR (400 MHz, CDCl₃) δ 8.51 (s, 1H), 7.86-7.74 (m, 2H), 7.67 (dd, J=8.0, 1.8 Hz, 1H), 7.62 (d, J=1.8 Hz, 1H), 7.45-7.31 (m, 2H), 6.78 (d, J=8.1 Hz, 1H), 4.01 (s, 2H), 3.97 (s, 3H); ¹⁹F NMR (376 MHz, CDCl₃) δ −58.04; ESIMS m/z 351 ([M+H]⁺).

### 2-Isopropyl-4-(1-(4-(trifluoromethoxy)phenyl)-1H-1,2,4-triazol-3-yl)aniline (C60)

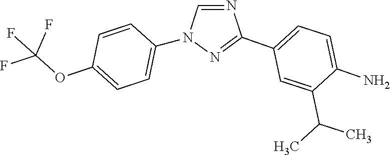 [SMILES: CC(C)c1cc(-c2ncn(-c3ccc(OC(F)(F)F)cc3)n2)ccc1N]

The title compound was prepared and was isolated as a yellow solid (500 mg, 42%): ¹H NMR (400 MHz, CDCl₃) δ 8.53 (d, J=15.8 Hz, 1H), 7.98 (d, J=2.0 Hz, 1H), 7.92-7.67 (m, 3H), 7.47-7.31 (m, 2H), 6.76 (d, J=8.2 Hz, 1H), 3.87 (s, 2H), 2.94 (hept, J=6.9 Hz, 1H), 1.35 (d, J=6.8 Hz, 6H); ¹⁹F NMR (376 MHz, CDCl₃) δ −58.04; ESIMS m/z 363 ([M+H]⁺).

### 2-Fluoro-4-(1-(4-(trifluoromethyl)phenyl)-1H-1,2,4-triazol-3-yl)aniline (C61)

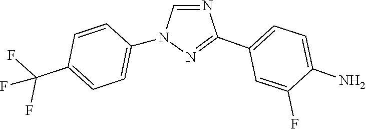 [SMILES: Nc1ccc(-c2ncn(-c3ccc(C(F)(F)F)cc3)n2)cc1F]

The title compound was prepared and was isolated as a pale yellow solid (659 mg, 59%): ¹H NMR (400 MHz, CDCl₃) δ 8.60 (s, 1H), 7.96-7.71 (m, 6H), 6.85 (dd, J=9.0, 8.2 Hz, 1H), 3.94 (s, 2H); ¹⁹F NMR (376 MHz, CDCl₃) δ −62.48, −135.33; HRMS-ESI (m/z) [M+H]⁺ calcd for C₁₅H₁₀F₄N₄, 323.0914; found, 323.0927.

### 2-Methyl-4-(1-(4-(trifluoromethyl)phenyl)-1H-1,2,4-triazol-3-yl)aniline (C62)

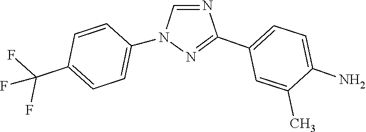 [SMILES: Cc1cc(-c2ncn(-c3ccc(C(F)(F)F)cc3)n2)ccc1N]

The title compound was prepared and was isolated as a white solid (328 mg, 59%): ¹H NMR (400 MHz, CDCl₃) δ 8.60 (s, 1H), 7.95-7.84 (m, 4H), 7.83-7.72 (m, 2H), 6.76 (d, J=8.1 Hz, 1H), 3.82 (s, 2H), 2.25 (s, 3H); ¹⁹F NMR (376 MHz, CDCl₃) δ−62.21, −62.40, −62.44, −62.49, −62.93; ESIMS m/z 319 ([M+H]⁺).

### 2-Chloro-4-(1-(4-(trifluoromethyl)phenyl)-1H-1,2,4-triazol-3-yl)aniline (C63)

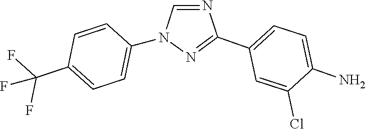 [SMILES: Nc1ccc(-c2ncn(-c3ccc(C(F)(F)F)cc3)n2)cc1Cl]

The title compound was prepared and was isolated as an off-white solid (1.00 g, 41%): ¹H NMR (400 MHz, CDCl₃) δ 8.60 (s, 1H), 8.13 (d, J=2.0 Hz, 1H), 7.94-7.84 (m, 3H), 7.82-7.74 (m, 2H), 6.85 (d, J=8.4 Hz, 1H), 4.27 (s, 2H); ESIMS m/z 339 ([M+H]⁺).

### 2-Fluoro-4-(1-(4-(2,2,2-trifluoroethoxy)phenyl)-1H-1,2,4-triazol-3-yl)aniline (C64)

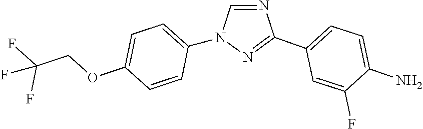 [SMILES: Nc1ccc(-c2ncn(-c3ccc(OCC(F)(F)F)cc3)n2)cc1F]

The title compound was prepared and was isolated as a yellow foam (244 mg, 63%): ¹H NMR (400 MHz, CDCl₃) δ 8.44 (s, 1H), 7.88-7.74 (m, 2H), 7.73-7.60 (m, 2H), 7.14-7.01 (m, 2H), 6.84 (t, J=8.6 Hz, 1H), 4.41 (q, J=8.0 Hz, 2H), 3.91 (s, 2H); ¹⁹F NMR (376 MHz, CDCl₃) δ−73.85, −135.43; ESIMS m/z 353 ([M+H]⁺).

### 2-Fluoro-4-(1-(4-(perfluoroethoxy)phenyl)-1H-1,2,4-triazol-3-yl)aniline (C65)

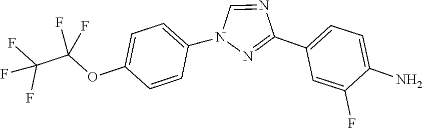 [SMILES: Nc1ccc(-c2ncn(-c3ccc(OC(F)(F)C(F)(F)F)cc3)n2)cc1F]

The title compound was prepared and was isolated as a yellow semi-solid (500 mg, 45%): ¹H NMR (400 MHz, CDCl₃) δ 8.52 (s, 1H), 7.87-7.73 (m, 4H), 7.47-7.32 (m, 2H), 6.85 (dd, J=9.0, 8.1 Hz, 1H), 3.93 (s, 2H); ¹⁹F NMR (376 MHz, CDCl₃) δ−85.91, −87.85, −135.37; ESIMS m/z 389 ([M+H]⁺).

### 2-Fluoro-4-(1-(4-((trifluoromethyl)thio)phenyl)-1H-1,2,4-triazol-3-yl)aniline (C66)

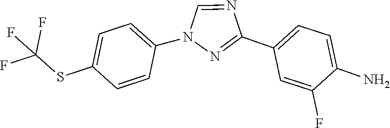 [SMILES: Nc1ccc(-c2ncn(-c3ccc(SC(F)(F)F)cc3)n2)cc1F]

The title compound was prepared and was isolated as an off-white fluffy powder (584 mg, 70%): ¹H NMR (400 MHz, CDCl₃) δ 8.59 (s, 1H), 7.88-7.75 (m, 6H), 6.85 (dd, J=9.0, 8.1 Hz, 1H), 3.94 (s, 2H); ¹⁹F NMR (376 MHz, CDCl₃) δ−42.69 (d, J=3.4 Hz), −42.69, −135.31; ESIMS m/z 355 ([M+H]⁺).

### 2-Methyl-4-(1-(4-((trifluoromethyl)thio)phenyl)-1H-1,2,4-triazol-3-yl)aniline (C67)

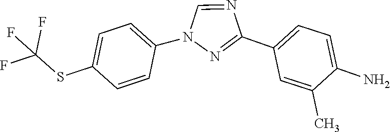 [SMILES: Cc1cc(-c2ncn(-c3ccc(SC(F)(F)F)cc3)n2)ccc1N]

The title compound was prepared and was isolated as a white solid (444 mg, 40%); ¹H NMR (400 MHz, CDCl₃) δ 8.58 (s, 1H), 8.50 (s, 1H), 7.83-7.81 (m, 3H), 7.77-7.72 (m, 2H), 6.76 (d, J=8.2 Hz, 1H), 3.83 (s, 2H), 2.25 (s, 3H); ¹⁹F NMR (376 MHz, CDCl₃) δ −42.47; ESIMS m/z 351 ([M+H]⁺).

### 2-Chloro-4-(1-(4-((trifluoromethyl)thio)phenyl)-1H-1,2,4-triazol-3-yl)aniline (C68)

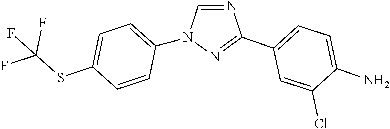 [SMILES: Nc1ccc(-c2ncn(-c3ccc(SC(F)(F)F)cc3)n2)cc1Cl]

The title compound was prepared and was isolated as a white solid (500 mg, 42%): ¹H NMR (400 MHz, CDCl₃) δ 8.59 (s, 1H), 8.12 (d, J=1.9 Hz, 1H), 7.90 (dd, J=8.3, 1.9 Hz, 1H), 7.86-7.74 (m, 4H), 6.85 (d, J=8.4 Hz, 1H), 4.27 (s, 2H); ¹⁹F NMR (376 MHz, CDCl₃) δ−42.68; ESIMS m/z 371 ([M+H]⁺).

### 2-Fluoro-4-(1-(5-(trifluoromethoxy)pyridin-2-yl)-1H-1,2,4-triazol-3-yl)aniline (C69)

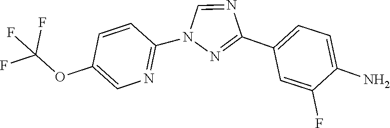 [SMILES: Nc1ccc(-c2ncn(-c3ccc(OC(F)(F)F)cn3)n2)cc1F]

The title compound was prepared and was isolated as a white solid (367 mg, 33%): ¹H NMR (400 MHz, CDCl₃) δ 9.10 (s, 1H), 8.48-8.30 (m, 1H), 8.03 (dd, J=8.9, 0.7 Hz, 1H), 7.91-7.69 (m, 3H), 6.86 (t, J=8.5 Hz, 1H), 3.94 (s, 2H); ¹⁹F NMR (376 MHz, CDCl₃) δ−58.40, −135.35; HRMS-ESI (m/z) [M+H]⁺ calcd for C₁₄H₉F₄N₅O, 340.0816; found, 340.0823.

### 2-Methyl-4-(1-(5-(trifluoromethoxy)pyridin-2-yl)-1H-1,2,4-triazol-3-yl)aniline (C70)

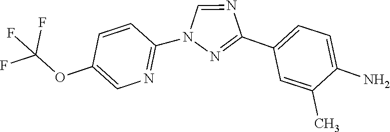 [SMILES: Cc1cc(-c2ncn(-c3ccc(OC(F)(F)F)cn3)n2)ccc1N]

The title compound was prepared and was isolated as an off-white solid (587 mg, 51%): ¹H NMR (400 MHz, CDCl₃) δ 9.10 (s, 1H), 8.38 (d, J=2.7 Hz, 1H), 8.04 (d, J=8.9 Hz, 1H), 7.99-7.81 (m, 1H), 7.76 (ddd, J=8.9, 2.8, 1.1 Hz, 1H), 7.36 (dt, J=4.7, 2.3 Hz, 1H), 6.76 (d, J=8.2 Hz, 1H), 3.83 (s, 2H), 2.25 (s, 3H); ¹⁹F NMR (376 MHz, CDCl₃) δ −58.40; HRMS-ESI (m/z) [M+H]⁺ calcd for C₁₅H₁₂F₃N₅O, 336.1067; found, 336.1074.

### 2-Chloro-4-(1-(5-(trifluoromethoxy)pyridin-2-yl)-1H-1,2,4-triazol-3-yl)aniline (C71)

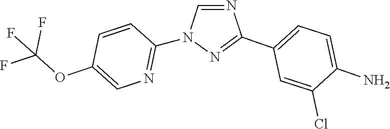 [SMILES: Nc1ccc(-c2ncn(-c3ccc(OC(F)(F)F)cn3)n2)cc1Cl]

The title compound was prepared and was isolated as an off-white solid (600 mg, 50%): ¹H NMR (400 MHz, CDCl₃) δ 9.11 (s, 1H), 8.39 (d, J=2.7 Hz, 1H), 8.13 (d, J=1.9 Hz, 1H), 8.04 (dd, J=8.9, 0.6 Hz, 1H), 7.91 (dd, J=8.3, 1.9 Hz, 1H), 7.85-7.73 (m, 1H), 6.85 (d, J=8.3 Hz, 1H), 4.29 (s, 2H); ¹⁹F NMR (376 MHz, CDCl₃) δ -58.39; ESIMS m/z 356 ([M+H]⁺).

### 2-Fluoro-4-(1-(5-(trifluoromethyl)pyridin-2-yl)-1H-1,2,4-triazol-3-yl)aniline (C72)

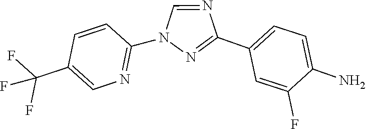 [SMILES: Nc1ccc(-c2ncn(-c3ccc(C(F)(F)F)cn3)n2)cc1F]

The title compound was prepared and was isolated as a white foam (900 mg, 78%): ¹H NMR (400 MHz, CDCl₃) δ 9.19 (s, 1H), 8.72 (dt, J=2.0, 1.0 Hz, 1H), 8.22-8.01 (m, 2H), 7.94-7.70 (m, 2H), 6.93-6.78 (m, 1H), 3.96 (s, 2H); ¹⁹F NMR (376 MHz, CDCl₃) δ−62.08, −135.32; ESIMS m/z 324 ([M+H]⁺).

### 2-Methyl-4-(1-(5-(trifluoromethyl)pyridin-2-yl)-1H-1,2,4-triazol-3-yl)aniline (C73)

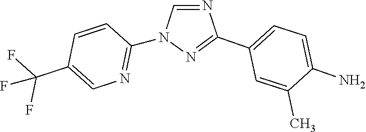 [SMILES: Cc1cc(-c2ncn(-c3ccc(C(F)(F)F)cn3)n2)ccc1N]

The title compound was prepared and was isolated as a yellow foam (925 mg, 81%): ¹H NMR (400 MHz, CDCl₃) δ 9.18 (s, 1H), 8.71 (dq, J=2.0, 1.1 Hz, 1H), 8.20-8.02 (m, 2H), 8.02-7.80 (m, 2H), 6.75 (d, J=8.2 Hz, 1H), 3.84 (s, 2H), 2.25 (s, 3H); ¹⁹F NMR (376 MHz, CDCl₃) δ −62.04; ESIMS m/z 320 ([M+H]⁺).

### 2-Chloro-4-(1-(5-(trifluoromethyl)pyridin-2-yl)-1H-1,2,4-triazol-3-yl)aniline (C74)

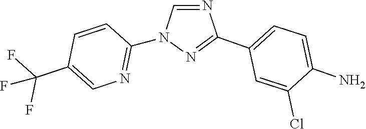 [SMILES: Nc1ccc(-c2ncn(-c3ccc(C(F)(F)F)cn3)n2)cc1Cl]

The title compound was prepared and was isolated as an off-white solid (240 mg, 39%): ¹H NMR (400 MHz, CDCl₃) δ 9.18 (s, 1H), 8.72 (dt, J=2.0, 0.9 Hz, 1H), 8.19-8.03 (m, 3H), 7.92 (dd, J=8.3, 1.9 Hz, 1H), 6.85 (d, J=8.3 Hz, 1H), 4.29 (s, 2H); ¹⁹F NMR (376 MHz, CDCl₃) δ−62.07; ESIMS m/z 340 ([M+H]⁺).

### 2-Fluoro-4-(1-(5-(trifluoromethyl)pyridin-2-yl)-1H-pyrazol-3-yl)aniline (C75)

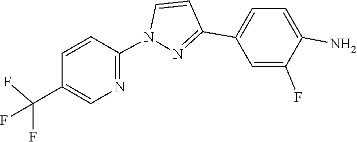 [SMILES: Nc1ccc(-c2ccn(-c3ccc(C(F)(F)F)cn3)n2)cc1F]

The title compound was prepared and was isolated as an off-white solid (976 mg, 87%): ¹H NMR (400 MHz, CDCl₃) δ 8.71-8.62 (m, 1H), 8.58 (d, J=2.8 Hz, 1H), 8.17 (d, J=8.7 Hz, 1H), 8.02 (dd, J=8.7, 2.4 Hz, 1H), 7.60 (dd, J=12.2, 1.9 Hz, 1H), 7.49 (dd, J=8.4, 1.9 Hz, 1H), 6.83 (dd, J=9.1, 8.2 Hz, 1H), 6.70 (d, J=2.7 Hz, 1H), 3.86 (s, 2H); ¹⁹F NMR (376 MHz, CDCl₃) δ−61.91, −135.22; ESIMS m/z 323 ([M+H]⁺).

### 2-Methyl-4-(1-(5-(trifluoromethyl)pyridin-2-yl)-1H-pyrazol-yl)aniline (C76)

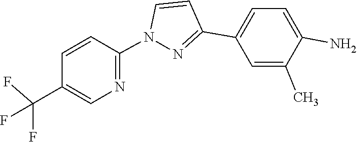 [SMILES: Cc1cc(-c2ccn(-c3ccc(C(F)(F)F)cn3)n2)ccc1N]

The title compound was prepared and was isolated as an off-white solid (937 mg, 84%): ¹H NMR (400 MHz, CDCl₃) δ 8.65 (dq, J=2.6, 0.9 Hz, 1H), 8.56 (d, J=2.7 Hz, 1H), 8.18 (dt, J=8.6, 0.8 Hz, 1H), 8.07-7.90 (m, 1H), 7.65 (dd, J=2.0, 0.9 Hz, 1H), 7.62-7.52 (m, 1H), 6.79-6.67 (m, 2H), 3.75 (s, 2H), 2.25 (s, 3H); ¹⁹F NMR (376 MHz, CDCl₃) δ −61.87; ESIMS m/z 317 ([M−H]⁺).

### Example 21: Preparation of 2-bromo-4-(1-(4-(trifluoromethoxy)phenyl)-1H-1,2,4-triazol-3-yl)aniline (C77)

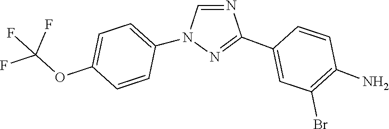 [SMILES: Nc1ccc(-c2ncn(-c3ccc(OC(F)(F)F)cc3)n2)cc1Br]

4-(1-(4-(Trifluoromethoxy)phenyl)-1H-1,2,4-triazol-3-yl)aniline (prepared as in PCT International Application Publication WO 2009102736 A1; 1.5 g, 4.68 mmol) was dissolved in acetonitrile (25 mL) and N-bromosuccinimide (0.917 g, 5.15 mmol) was added. The mixture was stirred for 2 min and was poured into water. The reaction mixture was extracted with EtOAc. The combined organic extracts were washed with brine, dried over Na₂SO₄, and concentrated. The resulting residue was loaded onto diatomaceous earth. Purification by silica gel chromatography eluting with 0-25% acetone-hexanes provided the title compound as a yellow solid (1.45 g, 77%): ¹H NMR (400 MHz, CDCl₃) δ 8.51 (s, 1H), 8.28 (d, J=1.9 Hz, 1H), 7.94 (dd, J=8.4, 1.9 Hz, 1H), 7.81-7.75 (m, 2H), 7.41-7.35 (m, 2H), 6.84 (d, J=8.3 Hz, 1H), 4.30 (s, 2H); ESIMS m/z 401 ([M+H]⁺).

### Example 22: Preparation of 4-(1-(4-(trifluoromethoxy)phenyl)-1H-1,2,4-triazol-3-yl)-2-(trifluoromethyl)aniline (C78)

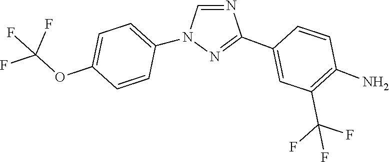 [SMILES: Nc1ccc(-c2ncn(-c3ccc(OC(F)(F)F)cc3)n2)cc1C(F)(F)F]

3-Bromo-1-(4-(trifluoromethoxy)phenyl)-1H-1,2,4-triazole (prepared as in Fischer at al., United States Patent Application Publication 20140274688 A1; 2.82 g, 7.77 mmol) was combined with 4-(4,4,5,5-tetramethyl-1,3,2-dioxaborolan-2-yl)-2-(trifluoromethyl)aniline (2.9 g, 10.10 mmol), K₃PO₄ (4.12 g, 19.43 mmol), and (2-dicyclohexylphosphino-2′,4′,6′-triisopropyl-1,1′-biphenyl)[2-(2′-amino-1,1′-biphenyl)]palladium(II) methanesulfonate (XPhos-G3-Palladacycle or XPhos-Pd-G3 precatalyst; 0.132 g, 0.155 mmol) in 10:1 dioxane-water (21.4 mL and 2.14 mL). The mixture was heated to 100° C. for 4 h. The mixture was cooled and filtered through diatomaceous earth. The resulting mixture was diluted with water and extracted with diethyl ether. The organic extracts were combined and concentrated. Purification by column chromatography eluting with 0-30% acetone-hexanes afforded the title compound as an off-white solid (2.11 g, 70%): ¹H NMR (300 MHz, CDCl₃) δ 8.53 (s, 1H), 8.29 (d, J=1.9 Hz, 1H), 8.11 (dd, J=8.2, 2.0 Hz, 1H), 7.83-7.77 (m, 2H), 7.41-7.36 (m, 2H), 6.83 (d, J=8.5 Hz, 1H), 4.38 (s, 2H); ESIMS m/z 389 ([M+H]⁺).

The following compounds were prepared in accordance to the procedure in Example 22.

### 2,6-Dimethyl-4-(1-(4-(trifluoromethoxy)phenyl)-1H-1,2,4-triazol-3-yl)aniline (C79)

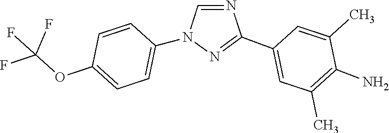 [SMILES: Cc1cc(-c2ncn(-c3ccc(OC(F)(F)F)cc3)n2)cc(C)c1N]

The title compound was prepared and was isolated as an off-white powder (246 mg, 22%): ¹H NMR (400 MHz, CDCl₃) δ 8.50 (s, 1H), 7.82-7.76 (m, 4H), 7.37 (d, J=8.6 Hz, 2H), 3.79 (s, 2H), 2.26 (s, 6H); ESIMS m/z 350 ([M+H]⁺).

### 2,6-Difluoro-4-(1-(4-(trifluoromethoxy)phenyl)-1H-1,2,4-triazol-3-yl)aniline (C80)

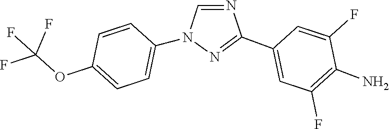 [SMILES: Nc1c(F)cc(-c2ncn(-c3ccc(OC(F)(F)F)cc3)n2)cc1F]

The title compound was prepared and was isolated as an off-white powder (350 mg, 50%): ¹H NMR (400 MHz, CDCl₃) δ 8.52 (s, 1H), 7.82-7.75 (m, 2H), 7.68 (dd, J=7.3, 2.1 Hz, 2H), 7.38 (dt, J=7.9, 1.0 Hz, 2H), 3.93 (s, 2H); ESIMS m/z 357 ([M+H]⁺).

### 3-(3-(Methoxymethoxy)-4-nitrophenyl)-1-(4-(trifluoromethoxy)phenyl)-1H-1,2,4-triazole (C81)

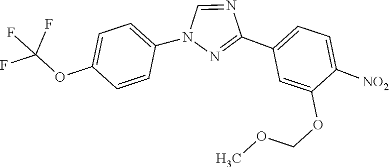 [SMILES: COCOc1cc(-c2ncn(-c3ccc(OC(F)(F)F)cc3)n2)ccc1[N+](=O)[O-]]

The title compound was prepared and was isolated as an off-white powder (1.34 g, 93%): ¹H NMR (300 MHz, CDCl₃) δ 8.60 (s, 1H), 8.14 (t, J=1.0 Hz, 1H), 7.94 (t, J=0.9 Hz, 2H), 7.86-7.77 (m, 2H), 7.41 (d, J=8.5 Hz, 2H), 5.43 (s, 2H), 3.58 (s, 3H); ESIMS m/z 411 ([M+H]⁺).

### 2-Amino-5-(1-(5-(trifluoromethoxy)pyridin-2-yl)-1H-1,2,4-triazol-3-yl)benzonitrile (C82)

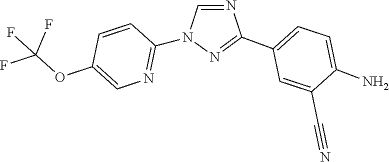 [SMILES: N#Cc1cc(-c2ncn(-c3ccc(OC(F)(F)F)cn3)n2)ccc1N]

The title compound was prepared and was isolated as an off-white powder (693 mg, 62%): ¹H NMR (300 MHz, CDCl₃) δ 9.12 (s, 1H), 8.40 (d, J=2.7 Hz, 1H), 8.28 (d, J=2.0 Hz, 1H), 8.17 (dd, J=8.6, 2.0 Hz, 1H), 8.03 (dd, J=8.9, 0.6 Hz, 1H), 7.85-7.73 (m, 1H), 6.85 (d, J=8.6 Hz, 1H), 4.63 (s, 2H); ESIMS m/z 347 ([M+H]⁺).

### 4-(1-(5-(Trifluoromethoxy)pyridin-2-yl)-1H-1,2,4-triazol-3-yl)-2-(trifluoromethyl)aniline (C83)

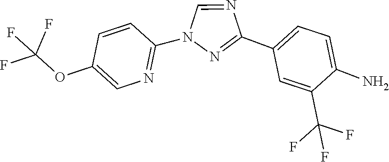 [SMILES: Nc1ccc(-c2ncn(-c3ccc(OC(F)(F)F)cn3)n2)cc1C(F)(F)F]

The title compound was prepared and was isolated as an off-white powder (672 mg, 53%): ¹H NMR (300 MHz, CDCl₃) δ 9.12 (s, 1H), 8.40 (d, J=2.8 Hz, 1H), 8.31 (d, J=1.9 Hz, 1H), 8.13 (dd, J=8.4, 2.0 Hz, 1H), 8.05 (dd, J=8.9, 0.6 Hz, 1H), 7.82-7.75 (m, 1H), 6.84 (d, J=8.5 Hz, 1H), 4.40 (s, 2H); ESIMS m/z 390 ([M+H]⁺).

### 2-(Trifluoromethyl)-4-(1-(5-(trifluoromethyl)pyridin-2-yl)-1H-1,2,4-triazol-3-yl)aniline (C84)

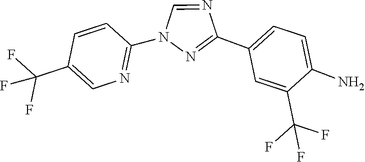 [SMILES: Nc1ccc(-c2ncn(-c3ccc(C(F)(F)F)cn3)n2)cc1C(F)(F)F]

The title compound was prepared and was isolated as an off-white solid (643 mg, 72%): ¹H NMR (400 MHz, CDCl₃) δ 9.20 (s, 1H), 8.73 (dt, J=2.0, 1.0 Hz, 1H), 8.32 (d, J=2.0 Hz, 1H), 8.17-8.08 (m, 3H), 6.84 (d, J=8.5 Hz, 1H), 4.41 (s, 2H); ESIMS m/z 375 ([M+H]⁺).

### 4-(3-(4-Amino-3-fluorophenyl)-1H-1,2,4-triazol-1-yl)benzonitrile (C85)

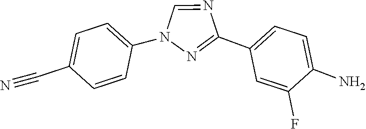 [SMILES: N#Cc1ccc(-n2cnc(-c3ccc(N)c(F)c3)n2)cc1]

The title compound was prepared and was isolated as an off-white powder (532 mg, 47%): ¹H NMR (400 MHz, CDCl₃) δ 8.62 (s, 1H), 7.93-7.88 (m, 2H), 7.85-7.77 (m, 4H), 6.89-6.83 (m, 1H), 3.96 (s, 2H); ESIMS m/z 280 ([M+H]⁺).

### Example 23: Preparation of 2-amino-5-(1-(4-(trifluoromethoxy)phenyl)-1H-1,2,4-triazol-3-yl)benzonitrile (C86)

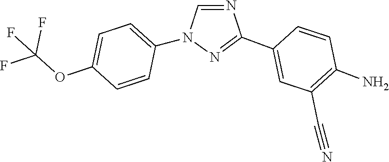 [SMILES: N#Cc1cc(-c2ncn(-c3ccc(OC(F)(F)F)cc3)n2)ccc1N]

2-Bromo-4-(1-(4-(trifluoromethoxy)phenyl)-1H-1,2,4-triazol-3-yl)aniline (C77; 0.510 g, 1.278 mmol) was dissolved in 1-methyl-2-pyrrolidinone (NMP; 3.65 mL) and copper cyanide (0.172 g, 1.92 mmol) was added. The mixture was heated to 155° C. for 24 h. The mixture was diluted with water and extracted with EtOAc. The organic extracts were washed with brine, then dried and concentrated. Purification on silica gel eluting with 0-30% acetone-hexanes afforded the title compound as an adduct with NMP and as a white solid (400 mg, 91%): ¹H NMR (400 MHz, CDCl₃) δ 8.53 (s, 1H), 8.26 (d, J=2.0 Hz, 1H), 8.15 (dd, J=8.6, 2.0 Hz, 1H), 7.81-7.75 (m, 2H), 7.39 (dq, J=8.0, 1.1 Hz, 2H), 6.84 (d, J=8.7 Hz, 1H), 4.63 (s, 2H); ESIMS m/z 346 ([M+H]⁺).

### Example 24: Preparation of 2-(methoxymethoxy)-4-(1-(4-(trifluoromethoxy)phenyl)-1H-1,2,4-triazol-3-yl)aniline (C87)

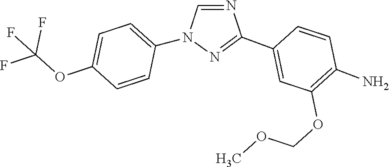 [SMILES: COCOc1cc(-c2ncn(-c3ccc(OC(F)(F)F)cc3)n2)ccc1N]

3-(3-(Methoxymethoxy)-4-nitrophenyl)-1-(4-(trifluoromethoxy)phenyl)-1H-1,2,4-triazole (C81; 1.34 g, 3.27 mmol) was dissolved in ethanol (8 mL) and EtOAc (8 mL), and palladium on carbon (5% w/w) (348 mg, 0.163 mmol) was added. The heterogeneous mixture was placed under an atmosphere of hydrogen and stirred at ambient temperature for 10 h. The reaction mixture was filtered and concentrated onto diatomaceous earth. Purification by silica gel chromatography eluting with 0-30% acetone-hexanes furnished the title compound as an off-white powder (980 mg, 79%): ¹H NMR (400 MHz, CDCl₃) δ 8.51 (s, 1H), 7.83 (d, J=1.8 Hz, 1H), 7.81-7.77 (m, 2H), 7.71 (dd, J=8.1, 1.8 Hz, 1H), 7.39-7.35 (m, 2H), 6.81 (d, J=8.1 Hz, 1H), 5.32 (s, 2H), 4.05 (s, 2H), 3.54 (s, 3H); ESIMS m/z 381 ([M+H]⁺).

### Example 25: Preparation of 2-methyl-4-(4,4,5,5-tetramethyl-1,3,2-dioxaborolan-2-yl)aniline (C88)

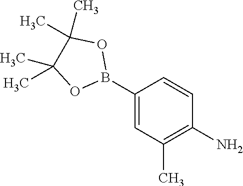 [SMILES: Cc1cc(B2OC(C)(C)C(C)(C)O2)ccc1N]

To a 250 mL round-bottomed flask were added 4-bromo-2-methylaniline (5.0 g, 26.9 mmol), 4,4,4′,4′,5,5,5′,5′-octamethyl-2,2′-bi(1,3,2-dioxaborolane) (8.96 g, 35.3 mmol), potassium acetate (5.27 g, 53.7 mmol), and dioxane (90 mL). The reaction mixture was degassed and backfilled with nitrogen (3×). [1,1′-Bis(diphenylphosphino)ferrocene]dichloropalladium(II) (0.98 g, 1.34 mmol) was added, and the reaction mixture was stirred at 90° C. for 3 h. The reaction mixture was cooled, concentrated, diluted with EtOAc and water. The mixture was extracted with EtOAc (3×). The organic extracts were washed with brine, dried over anhydrous Na₂SO₄, filtered, and concentrated under reduced pressure. Purification by column chromatography afforded the title compound as a light yellow semi-solid (4.12 g, 63%): 1H NMR (400 MHz, CDCl₃) δ 7.55-7.47 (m, 2H), 6.65 (d, J=7.7 Hz, 1H), 3.78 (s, 2H), 2.16 (s, 3H), 1.32 (s, 12H); ¹³C NMR (101 MHz, CDCl₃) δ 147.62, 137.28, 134.17, 132.85, 129.58, 121.12, 116.34, 114.02, 110.09, 83.50, 83.26, 25.04, 24.85, 21.04, 17.19, 16.99, 14.21.

### Example 26: Preparation of 2,6-difluoro-4-(4,4,5,5-tetramethyl-1,3,2-dioxaborolan-2-yl)aniline (C89)

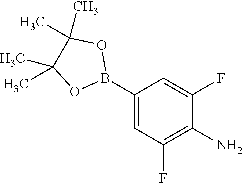 [SMILES: CC1(C)OB(c2cc(F)c(N)c(F)c2)OC1(C)C]

Bis(pinacolato)diboron (1.34 g, 5.29 mmol), 4-bromo-2,6-difluoroaniline (1 g, 4.81 mmol), potassium acetate (1.415 g, 14.42 mmol), and [1,1′-bis(diphenylphosphino)ferrocene]dichloropalladium(II) (0.088 g, 0.120 mmol) were combined in DMSO (9.62 mL) and heated to 90° C. overnight. The mixture was then poured into water and extracted with ether. The organic extracts were washed with brine, then dried, and concentrated to afford a yellow oil. Purification by silica gel chromatography eluting with 0-30% EtOAc-hexanes yielded the title compound as a clear oil that solidified upon standing (1.1 g, 90%): ¹H NMR (400 MHz, CDCl₃) δ 7.24 (dd, J=6.8, 2.2 Hz, 2H), 3.93 (s, 2H), 1.32 (s, 12H); ESIMS m/z 256 ([M+H]⁺).

The following compound was prepared in accordance to the procedure in Example 26.

### 2-(3-(Methoxymethoxy)-4-nitrophenyl)-4,4,5,5-tetramethyl-1,3,2-dioxaborolane (C90)

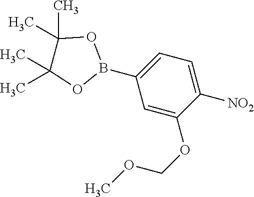 [SMILES: COCOc1cc(B2OC(C)(C)C(C)(C)O2)ccc1[N+](=O)[O-]]

The title compound was prepared and was isolated as an off-white solid (2.9 g, 72%): ¹H NMR (400 MHz, CDCl₃) δ 7.74 (d, J=8.0 Hz, 1H), 7.66 (d, J=1.1 Hz, 1H), 7.50 (dd, J=8.0, 1.1 Hz, 1H), 5.33 (s, 2H), 3.54 (s, 3H), 1.35 (s, 12H); EIMS m/z 309.

### Example 27: Preparation of 4-bromo-2-(methoxymethoxy)-1-nitrobenzene (C91)

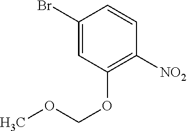

5-Bromo-2-nitrophenol (3 g, 13.8 mmol) was dissolved in DMF (55.0 mL) at room temperature, and added in portions was sodium hydride (60% in mineral oil; 0.660 g, 16.5 mmol). The bright yellow solution was stirred for 60 min, and chloromethyl methyl ether (1.10 mL, 14.5 mmol) was added. The mixture was stirred for 16 h, poured into water, and extracted with ether. The combined organic extracts were washed with brine, dried over Na₂SO₄, and concentrated. Purification on silica gel eluting with 0-25% acetone-hexanes afforded the desired compound as a yellow crystalline solid (3.4 g, 94%): EIMS m/z 263.

### Example 28: Preparation of 4-(3-bromo-1H-1,2,4-triazol-1-yl)benzonitrile (C92)

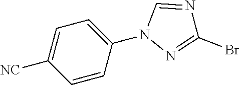 [SMILES: N#Cc1ccc(-n2cnc(Br)n2)cc1]

4-Fluorobenzonitrile (1.5 g, 12.4 mmol) and 3-bromo-1H-1,2,4-triazole (1.833 g, 12.4 mmol) were combined in DMF (37.5 mL), and potassium carbonate (1.712 g, 12.38 mmol) was added in portions. The mixture was then heated to 95° C. overnight. The mixture was cooled to room temperature, and water was added. A white precipitate resulted. The solid was rinsed with water, then diethyl ether, and then placed under vacuum at 55° C. for 4 h. The title compound was used without further purification (2.9 g, 94%): ¹H NMR (500 MHz, CDCl₃) δ 8.54 (s, 1H), 7.88-7.79 (m, 4H); EIMS m/z 250.

### Example 29: Preparation of 3-bromo-1-(4-(pentafluoro-λ⁶-sulfaneyl)phenyl)-1H-1,2,4-triazole (C93)

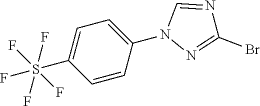 [SMILES: FS(F)(F)(F)(F)c1ccc(-n2cnc(Br)n2)cc1]

To a 250 mL round-bottomed flask were added 3-bromo-1H-1,2,4-triazole (3.92 g, 26.5 mmol), cesium carbonate (8.63 g, 26.5 mmol) and DMSO (58.9 mL). (4-Bromophenyl)pentafluoro-λ⁶-sulfane (5 g, 17.7 mmol) and copper(I) iodide (0.673 g, 3.53 mmol) were added sequentially. The reaction mixture was pumped and purged with nitrogen gas and heated to an internal temperature of 130° C. overnight. The reaction mixture was allowed to cool to room temperature and was diluted with EtOAc and a small amount of water. The mixture was extracted with EtOAc (3×). The organic extracts were combined, washed with brine, dried over Na₂SO₄, filtered, and concentrated under reduced pressure. Purification by column chromatography afforded the title compound as a white solid (1.54 g, 24%): ¹H NMR (400 MHz, CDCl₃) δ 8.52 (s, 1H), 7.98-7.87 (m, 2H), 7.79 (d, J=8.8 Hz, 2H).

The following compounds were prepared in accordance to the procedure in Example 29.

### 3-Bromo-1-(4-(trifluoromethoxy)phenyl)-1H-pyrazole (C94)

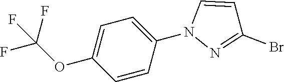 [SMILES: FC(F)(F)Oc1ccc(-n2ccc(Br)n2)cc1]

The title compound was prepared and was isolated as a yellow oil (1.08 g, 51%): ¹H NMR (300 MHz, CDCl₃) δ 7.79 (dd, J=2.5, 0.9 Hz, 1H), 7.75-7.61 (m, 2H), 7.31 (ddt, J=7.9, 2.0, 1.0 Hz, 2H), 6.50 (dd, J=2.5, 0.8 Hz, 1H); HRMS-ESI (m/z) [M+H]⁺ calcd for C₁₀H₆BrF₃N₂O, 305.9616; found, 306.9685.

### 2-(3-Bromo-1H-1,2,4-triazol-1-yl)-5-(trifluoromethyl)pyridine (C95)

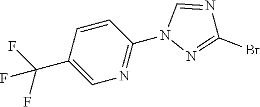 [SMILES: FC(F)(F)c1ccc(-n2cnc(Br)n2)nc1]

The title compound was prepared and was isolated as a white solid (2.8 g, 41%): ¹H NMR (300 MHz, CDCl₃) δ 9.11 (s, 1H), 8.74 (dt, J=2.5, 0.9 Hz, 1H), 8.15 (ddd, J=8.6, 2.4, 0.7 Hz, 1H), 8.01 (dt, J=8.6, 0.8 Hz, 1H); ¹⁹F NMR (376 MHz, CDCl₃) δ −61.95-−62.48 (m), −62.72 (d, J=22.2 Hz).

### 2-(3-Bromo-1H-pyrazol-1-yl)-5-(trifluoromethyl)pyridine (C96)

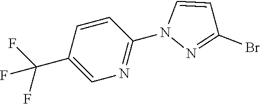 [SMILES: FC(F)(F)c1ccc(-n2ccc(Br)n2)nc1]

The title compound was prepared and was isolated as a white solid (3.4 g, 68%): ¹H NMR (400 MHz, CDCl₃) δ 8.67 (dt, J=2.2, 1.0 Hz, 1H), 8.50 (d, J=2.7 Hz, 1H), 8.11-7.99 (m, 2H), 6.53 (d, J=2.7 Hz, 1H); ¹⁹F NMR (376 MHz, CDCl₃) δ −62.04.

### 2-(3-Bromo-1H-pyrazol-1-yl)-5-(trifluoromethoxy)pyridine (C97)

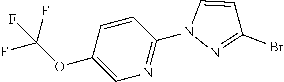 [SMILES: FC(F)(F)Oc1ccc(-n2ccc(Br)n2)nc1]

The title compound was prepared and was isolated as a white solid (4 g, 60%): ¹H NMR (400 MHz, CDCl₃) δ 8.42 (d, J=2.6 Hz, 1H), 8.33 (d, J=2.7 Hz, 1H), 8.01 (dd, J=9.0, 0.7 Hz, 1H), 7.75-7.62 (m, 1H), 6.50 (d, J=2.6 Hz, 1H); ¹⁹F NMR (376 MHz, CDCl₃) δ −58.44.

### Example 30: Preparation of (Z)-1-(3-(2-ethyl-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J16)

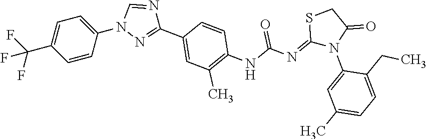 [SMILES: CCc1ccc(C)cc1N1C(=O)CS/C1=N\C(=O)Nc1ccc(-c2ncn(-c3ccc(C(F)(F)F)cc3)n2)cc1C]

2-Methyl-4-(1-(4-(trifluoromethyl)phenyl)-1H-1,2,4-triazol-3-yl)benzoyl azide (C21; 149 mg, 0.38 mmol) in acetonitrile (1.9 mL) was heated at 80° C. for 2 h. The reaction mixture was cooled, and 1-(2-ethyl-5-methylphenyl)thiourea (prepared as in U.S. Patent Application Publication 20140274688 A1; 76 mg, 0.39 mmol) and cesium carbonate (280 mg, 0.86 mmol) were added. The reaction mixture was stirred at room temperature for 3 d. Ethanol (1.9 mL), sodium acetate (63 mg, 0.77 mmol) and methyl 2-bromoacetate (0.05 mL, 0.50 mmol) were added, and the reaction mixture was heated at 60° C. for 4 h. The reaction mixture was cooled to room temperature, diluted with EtOAc and washed with water. The organic layers were dried over Na₂SO₄, filtered and concentrated. Purification of the residue by flash chromatography (eluting with EtOAc-1:1 hexanes-DCM), followed by reverse-phase flash chromatography (0-100% acetonitrile-water) provided the title compound as an off-white solid (38 mg, 17%).

The following compounds were prepared in accordance to the procedure in Example 30.

### (Z)-1-(3-(2-Isopropyl-5-methoxyphenyl)-4-oxothiazolidin-2-ylidene)-3-(2-methyl-4-(1-(4-(perfluoroethoxy)phenyl)-1H-1,2,4-triazol-3-yl)phenyl)urea (J2)

The title compound was prepared and was isolated as an orange solid (25 mg, 70%).

### (Z)-1-(3-(2-Isopropyl-5-methylphenyl)-4-oxothiazolidin-2-ylidene)-3-(2-methoxy-4-(1-(4-(trifluoromethoxy)phenyl)-1H-1,2,4-triazol-3-yl)phenyl)urea (J3)

The title compound was prepared and was isolated as a dark brown oil (40 mg, 32%).

### (Z)-1-(3-(2-Isopropyl-5-methylphenyl)-4-oxothiazolidin-2-ylidene)-3-(3-methyl-4-(1-(4-(trifluoromethyl)phenyl)-1H-1,2,4-triazol-3-yl)phenyl)urea (J4)

The title compound was prepared and was isolated as a dark brown solid (150 mg, 55%).

### (Z)-1-(3-(2-Isopropyl-5-methoxyphenyl)-4-oxothiazolidin-2-ylidene)-3-(3-methyl-4-(1-(4-(trifluoromethyl)phenyl)-1H-1,2,4-triazol-3-yl)phenyl)urea (J5)

The title compound was prepared and was isolated as a yellow solid (80 mg, 38%).

### (Z)-1-(3-(2-Isopropyl-5-methoxyphenyl)-4-oxothiazolidin-2-ylidene)-3-(2-methoxy-4-(1-(4-(trifluoromethoxy)phenyl)-1H-1,2,4-triazol-3-yl)phenyl)urea (J6)

The title compound was prepared and was isolated as an orange solid (16 mg, 13%).

### (Z)-1-(3-(2-Isopropyl-5-methoxyphenyl)-4-oxothiazolidin-2-ylidene)-3-(3-methyl-4-(1-(4-(perfluoroethoxy)phenyl)-1H-1,2,4-triazol-3-yl)phenyl)urea (J7)

The title compound was prepared and was isolated as a yellow solid (23 mg, 21%).

### (Z)-1-(3-(2-Isopropyl-5-methoxyphenyl)-4-oxothiazolidin-2-ylidene)-3-(2-methoxy-4-(1-(4-(trifluoromethyl)phenyl)-1H-1,2,4-triazol-3-yl)phenyl)urea (J10)

The title compound was prepared and was isolated as a brown solid (19 mg, 11%).

### (Z)-1-(3-(5-Methyl-2-propylphenyl)-4-oxothiazolidin-2-ylidene)-3-(3-methyl-4-(1-(4-(perfluoroethoxy)phenyl)-1H-1,2,4-triazol-3-yl)phenyl)urea (J12)

The title compound was prepared and was isolated as an orange solid (70 mg, 31%).

### (Z)-1-(3-(2-Isopropyl-5-methylphenyl)-4-oxothiazolidin-2-ylidene)-3-(2-methoxy-4-(1-(4-(trifluoromethyl)phenyl)-1H-1,2,4-triazol-3-yl)phenyl)urea (J13)

The title compound was prepared and was isolated as a tan solid (18 mg, 11%).

### (Z)-1-(3-(2-Ethyl-5-methylphenyl)-4-oxothiazolidin-2-ylidene)-3-(3-methyl-4-(1-(4-(perfluoroethoxy)phenyl)-1H-1,2,4-triazol-3-yl)phenyl)urea (J14)

The title compound was prepared and was isolated as a red oil (47 mg, 22%).

### (Z)-1-(3-(5-Methyl-2-propylphenyl)-4-oxothiazolidin-2-ylidene)-3-(2-methyl-4-(1-(4-(trifluoromethyl)phenyl)-1H-1,2,4-triazol-3-yl)phenyl)urea (J15)

The title compound was prepared and was isolated as a tan solid (39 mg, 13%).

### (Z)-1-(3-(2-Isopropyl-5-methoxyphenyl)-4-oxothiazolidin-2-ylidene)-3-(2-methyl-4-(1-(4-(trifluoromethyl)phenyl)-1H-1,2,4-triazol-3-yl)phenyl)urea (J18)

The title compound was prepared and was isolated as a tan solid (34 mg, 15%).

### (Z)-1-(2-Fluoro-4-(1-(4-(trifluoromethyl)phenyl)-1H-1,2,4-triazol-3-yl)phenyl)-3-(3-(5-methyl-2-propylphenyl)-4-oxothiazolidin-2-ylidene)urea (J20)

The title compound was prepared and was isolated as an orange solid (60 mg, 26%).

### (Z)-1-(2-Fluoro-4-(1-(4-(trifluoromethyl)phenyl)-1H-1,2,4-triazol-3-yl)phenyl)-3-(3-(2-isopropyl-5-methoxyphenyl)-4-oxothiazolidin-2-ylidene)urea (J21)

The title compound was prepared and was isolated as a tan solid (33 mg, 15%).

### (Z)-1-(3-(2-Isopropyl-5-methoxyphenyl)-4-oxothiazolidin-2-ylidene)-3-(3-methyl-4-(1-(4-(trifluoromethoxy)phenyl)-1H-1,2,4-triazol-3-yl)phenyl)urea (J22)

The title compound was prepared and was isolated as a yellow solid (36 mg, 15%).

### (Z)-1-(3-(2-Isopropyl-5-methylphenyl)-4-oxothiazolidin-2-ylidene)-3-(3-methyl-4-(1-(4-(trifluoromethoxy)phenyl)-1H-1,2,4-triazol-3-yl)phenyl)urea (J23)

The title compound was prepared and was isolated as a yellow solid (31 mg, 13%).

### (Z)-1-(3-(2-Isopropyl-5-methylphenyl)-4-oxothiazolidin-2-ylidene)-3-(2-methyl-4-(1-(4-(perfluoroethoxy)phenyl)-1H-1,2,4-triazol-3-yl)phenyl)urea (J24)

The title compound was prepared and was isolated as a tan solid (68 mg, 32%).

### (Z)-1-(3-(2-Isopropyl-5-methoxyphenyl)-4-oxothiazolidin-2-ylidene)-3-(2-methyl-4-(1-(4-(perfluoroethoxy)phenyl)-1H-1,2,4-triazol-3-yl)phenyl)urea (J25)

The title compound was prepared and was isolated as a pink solid (86 mg, 39%).

### (Z)-1-(3-(2-Isopropyl-5-methylphenyl)-4-oxothiazolidin-2-ylidene)-3-(2-methoxy-4-(1-(4-(perfluoroethoxy)phenyl)-1H-1,2,4-triazol-3-yl)phenyl)urea (J28)

The title compound was prepared and was isolated as an orange solid (28 mg, 22%).

### (Z)-1-(3-(2-Isopropyl-5-methoxyphenyl)-4-oxothiazolidin-2-ylidene)-3-(2-methoxy-4-(1-(4-(perfluoroethoxy)phenyl)-1H-1,2,4-triazol-3-yl)phenyl)urea (J29)

The title compound was prepared and was isolated as an orange solid (29 mg, 21%).

### Example 31: Preparation of (Z)-1-(3-(2-isopropyl-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J1)

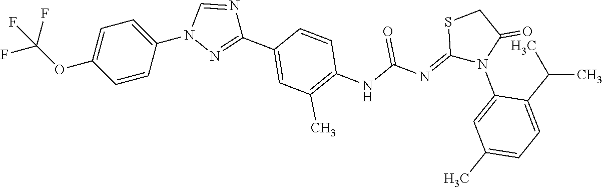 [SMILES: Cc1ccc(C(C)C)c(N2C(=O)CS/C2=N\C(=O)Nc2ccc(-c3ncn(-c4ccc(OC(F)(F)F)cc4)n3)cc2C)c1]

To 2-methyl-4-(1-(4-(trifluoromethoxy)phenyl)-1H-1,2,4-triazol-3-yl)aniline (C56; 100 mg, 0.299 mmol) and bis(2,5-dioxopyrrolidin-1-yl) carbonate (92 mg, 0.359 mmol) in DCM (1.021 mL) was added pyridine (0.029 mL, 0.359 mmol) and the reaction mixture was stirred at room temperature for 30 min. 2-Imino-3-(2-isopropyl-5-methylphenyl)thiazolidin-4-one (prepared as in U.S. Patent Application Publication 20140274688 A1; 74.3 mg, 0.299 mmol), sodium carbonate (201 mg, 2.393 mmol) and water (1.021 mL) were added sequentially, and the reaction mixture was allowed to continue stirring at room temperature overnight. The reaction mixture was filtered through a phase separator, rinsing with DCM. The filtrate was concentrated onto silica. Purification via flash chromatography (silica; EtOAc-hexanes) yielded the title compound as an orange solid (128 mg, 69%).

The following compounds were prepared in accordance to the procedure in Example 31.

### (Z)-1-(3-(2-Isopropyl-5-methoxyphenyl)-4-oxothiazolidin-2-ylidene)-3-(2-methyl-4-(1-(4-(trifluoromethyl)phenyl)-1H-1,2,4-triazol-3-yl)phenyl)urea (J8)

The title compound was prepared from 2-imino-3-(2-isopropyl-5-methoxyphenyl)thiazolidin-4-one (C98) and was isolated as a light yellow solid (32 mg, 32%).

### (Z)-1-(3-(2-Isopropyl-5-methylphenyl)-4-oxothiazolidin-2-ylidene)-3-(2-methyl-4-(1-(4-(trifluoromethyl)phenyl)-1H-1,2,4-triazol-3-yl)phenyl)urea (J9)

The title compound was prepared from 2-imino-3-(2-isopropyl-5-methylphenyl)thiazolidin-4-one (prepared as in U.S. Patent Application Publication 20140274688 A1) and was isolated as a light yellow film (11 mg, 11%).

### (Z)-1-(2-Fluoro-4-(1-(4-(trifluoromethyl)phenyl)-1H-1,2,4-triazol-3-yl)phenyl)-3-(3-(2-isopropyl-5-methoxyphenyl)-4-oxothiazolidin-2-ylidene)urea (J11)

The title compound was prepared from 2-imino-3-(2-isopropyl-5-methoxyphenyl)thiazolidin-4-one (C98) and was isolated as a pale orange solid (72 mg, 74%).

### (Z)-1-(2-Fluoro-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J19)

The title compound was prepared from 2-imino-3-(2-isopropyl-5-methylphenyl)thiazolidin-4-one (prepared as in U.S. Patent Application Publication 20140274688 A1) and was isolated as a pale yellow solid (132 mg, 46%).

### (Z)-1-(2-Fluoro-4-(1-(4-(perfluoroethoxy)phenyl)-1H-1,2,4-triazol-3-yl)phenyl)-3-(3-(2-isopropyl-5-methylphenyl)-4-oxothiazolidin-2-ylidene)urea (J26)

The title compound was prepared from 2-imino-3-(2-isopropyl-5-methylphenyl)thiazolidin-4-one (prepared as in U.S. Patent Application Publication 20140274688 A1) and was isolated as a white solid (32 mg, 18%).

### (Z)-1-(2-Fluoro-4-(1-(4-(perfluoroethoxy)phenyl)-1H-1,2,4-triazol-3-yl)phenyl)-3-(3-(2-isopropyl-5-methoxyphenyl)-4-oxothiazolidin-2-ylidene)urea (J27)

The title compound was prepared from 2-imino-3-(2-isopropyl-5-methoxyphenyl)thiazolidin-4-one (C98) and was isolated as a pale purple solid (64 mg, 35%).

### (Z)-1-(2-Fluoro-4-(1-(4-(trifluoromethoxy)phenyl)-1H-1,2,4-triazol-3-yl)phenyl)-3-(3-(5-methyl-2-(trifluoromethyl)phenyl)-4-oxothiazolidin-2-ylidene)urea (J30)

The title compound was prepared from 2-imino-3-(5-methyl-2-(trifluoromethyl)phenyl)thiazolidin-4-one (prepared as in PCT International Application Publication WO 2017040742 A1) and was isolated as an orange foam (150 mg, 50%).

### (Z)-1-(2-Fluoro-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J31)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as a yellow solid (181 mg, 58%).

### (Z)-1-(2-Fluoro-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J35)

The title compound was prepared from 2-imino-3-(5-methyl-2-(3,3,3-trifluoropropoxy)phenyl)thiazolidin-4-one (C99) and was isolated as an orange foam (250 mg, 78%).

### (Z)-1-(2-Chloro-4-(1-(4-(trifluoromethoxy)phenyl)-1H-1,2,4-triazol-3-yl)phenyl)-3-(3-(2-isopropyl-5-methoxyphenyl)-4-oxothiazolidin-2-ylidene)urea (J37)

The title compound was prepared from 2-imino-3-(2-isopropyl-5-methoxyphenyl)thiazolidin-4-one (C98) and was isolated as an off-white solid (136 mg, 47%).

### (Z)-1-(2-Chloro-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J38)

The title compound was prepared from 2-imino-3-(2-isopropyl-5-methylphenyl)thiazolidin-4-one (prepared as in U.S. Patent Application Publication 20140274688 A1) and was isolated as an off-white solid (214 mg, 77%).

### (Z)-1-(2-Chloro-4-(1-(4-(trifluoromethoxy)phenyl)-1H-1,2,4-triazol-3-yl)phenyl)-3-(3-(5-methyl-2-(trifluoromethyl)phenyl)-4-oxothiazolidin-2-ylidene)urea (J39)

The title compound was prepared from 2-imino-3-(5-methyl-2-(trifluoromethyl)phenyl)thiazolidin-4-one (prepared as in PCT International Application Publication WO 2017040742 A1) and was isolated as an off-white solid (174 mg, 62%).

### (Z)-1-(2-Chloro-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J40)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white solid (151 mg, 51%).

### (Z)-1-(2-Fluoro-4-(1-(4-(trifluoromethoxy)phenyl)-1H-1,2,4-triazol-3-yl)phenyl)-3-(3-(5-methyl-2-propylphenyl)-4-oxothiazolidin-2-ylidene)urea (J41)

The title compound was prepared from 2-imino-3-(5-methyl-2-propylphenyl)thiazolidin-4-one (prepared as in PCT International Application Publication WO 2016033025 A1) and was isolated as an orange foam (58 mg, 20%).

### (Z)-1-(3-(5-Chloro-2-(trifluoromethyl)phenyl)-4-oxothiazolidin-2-ylidene)-3-(2-chloro-4-(1-(4-(trifluoromethoxy)phenyl)-1H-1,2,4-triazol-3-yl)phenyl)urea (J42)

The title compound was prepared from 3-(5-chloro-2-(trifluoromethyl)phenyl)-2-iminothiazolidin-4-one (C5) and was isolated as an off-white solid (116 mg, 40%).

### (Z)-1-(2-Fluoro-4-(1-(4-(trifluoromethoxy)phenyl)-1H-1,2,4-triazol-3-yl)phenyl)-3-(3-(5-methoxy-2-propylphenyl)-4-oxothiazolidin-2-ylidene)urea (J43)

The title compound was prepared from 2-imino-3-(5-methoxy-2-propylphenyl)thiazolidin-4-one (C113) and was isolated as an off-white solid (135 mg, 69%).

### (Z)-1-(3-(2-Butyl-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J44)

The title compound was prepared from 3-(2-butyl-5-methylphenyl)-2-iminothiazolidin-4-one (C114) and was isolated as an orange foam (40 mg, 22%).

### (Z)-1-(3-(2-Butyl-5-methoxyphenyl)-4-oxothiazolidin-2-ylidene)-3-(2-fluoro-4-(1-(4-(trifluoromethoxy)phenyl)-1H-1,2,4-triazol-3-yl)phenyl)urea (J45)

The title compound was prepared from 3-(2-butyl-5-methoxyphenyl)-2-iminothiazolidin-4-one (C116) and was isolated as an orange foam (80 mg, 42%).

### (Z)-1-(3-(5-Chloro-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J46)

The title compound was prepared from 3-(5-chloro-2-isopropylphenyl)-2-iminothiazolidin-4-one (prepared as in PCT International Application Publication WO 2016033025 A1) and was isolated as a pale yellow solid (112 mg, 56%).

### (Z)-1-(2-Fluoro-4-(1-(4-(trifluoromethoxy)phenyl)-1H-1,2,4-triazol-3-yl)phenyl)-3-(3-(2-isobutyl-5-methylphenyl)-4-oxothiazolidin-2-ylidene)urea (J47)

The title compound was prepared from 2-imino-3-(2-isobutyl-5-methylphenyl) thiazolidin-4-one (C122) and was isolated as a pale yellow solid (101 mg, 51%).

### (Z)-1-(3-(2-(sec-Butyl)-5-methoxyphenyl)-4-oxothiazolidin-2-ylidene)-3-(2-fluoro-4-(1-(4-(trifluoromethoxy)phenyl)-1H-1,2,4-triazol-3-yl)phenyl)urea (J48)

The title compound was prepared from 3-(2-(sec-butyl)-5-methoxyphenyl)-2-iminothiazolidin-4-one (C123) and was isolated as a pale yellow solid (120 mg, 63%).

### (Z)-1-(2-Fluoro-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J49)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow solid (70 mg, 35%).

### (Z)-1-(3-(2,5-Bis(trifluoromethyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J50)

The title compound was prepared from 3-(2,5-bis(trifluoromethyl)phenyl)-2-iminothiazolidin-4-one (C137) and was isolated as a white solid (40 mg, 18%).

### (Z)-1-(3-(2-(sec-Butoxy)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J51)

The title compound was prepared from 3-(2-(sec-butoxy)-5-methylphenyl)-2-iminothiazolidin-4-one (C146) and was isolated as a light yellow film (42 mg, 21%).

### (Z)-1-(2-Fluoro-4-(1-(4-(perfluoroethoxy)phenyl)-1H-1,2,4-triazol-3-yl)phenyl)-3-(3-(5-methyl-2-propylphenyl)-4-oxothiazolidin-2-ylidene)urea (J52)

The title compound was prepared from 2-imino-3-(5-methyl-2-propylphenyl)thiazolidin-4-one (prepared as in PCT International Application Publication WO 2016033025 A1) and was isolated as a white solid (20 mg, 11%).

### (Z)-1-(3-(5-Methyl-2-propylphenyl)-4-oxothiazolidin-2-ylidene)-3-(2-methyl-4-(1-(4-(trifluoromethoxy)phenyl)-1H-1,2,4-triazol-3-yl)phenyl)urea (J53)

The title compound was prepared from 2-imino-3-(5-methyl-2-propylphenyl)thiazolidin-4-one (prepared as in PCT International Application Publication WO 2016033025 A1) and was isolated as an orange solid (64 mg, 34%).

### (Z)-1-(3-(2-Isopropyl-5-methoxyphenyl)-4-oxothiazolidin-2-ylidene)-3-(2-methyl-4-(1-(4-(trifluoromethoxy)phenyl)-1H-1,2,4-triazol-3-yl)phenyl)urea (J54)

The title compound was prepared from 2-imino-3-(2-isopropyl-5-methoxyphenyl)thiazolidin-4-one (C98) and was isolated as an orange solid (135 mg, 71%).

### (Z)-1-(3-(5-Methyl-2-(2,2,2-trifluoroethoxy)phenyl)-4-oxothiazolidin-2-ylidene)-3-(2-methyl-4-(1-(4-(trifluoromethoxy)phenyl)-1H-1,2,4-triazol-3-yl)phenyl)urea (J55)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white solid (144 mg, 71%).

### (Z)-1-(3-(5-Methyl-2-(3,3,3-trifluoropropoxy)phenyl)-4-oxothiazolidin-2-ylidene)-3-(2-methyl-4-(1-(4-(trifluoromethoxy)phenyl)-1H-1,2,4-triazol-3-yl)phenyl)urea (J56)

The title compound was prepared from 2-imino-3-(5-methyl-2-(3,3,3-trifluoropropoxy)phenyl)thiazolidin-4-one (C99) and was isolated as an orange solid (164 mg, 79%).

### (Z)-1-(2-Fluoro-4-(1-(4-(2,2,2-trifluoroethoxy)phenyl)-1H-1,2,4-triazol-3-yl)phenyl)-3-(3-(2-isopropyl-5-methylphenyl)-4-oxothiazolidin-2-ylidene)urea (J57)

The title compound was prepared from 2-imino-3-(2-isopropyl-5-methylphenyl)-thiazolidin-4-one (prepared as in U.S. Patent Application Publication 20140274688 A1) and was isolated as an orange solid (68 mg, 73%).

### (Z)-1-(2-Fluoro-4-(1-(4-(2,2,2-trifluoroethoxy)phenyl)-1H-1,2,4-triazol-3-yl)phenyl)-3-(3-(2-isopropyl-5-methoxyphenyl)-4-oxothiazolidin-2-ylidene)urea (J58)

The title compound was prepared from 2-imino-3-(2-isopropyl-5-methoxyphenyl)thiazolidin-4-one (C98) and was isolated as an orange solid (46 mg, 49%).

### (Z)-1-(3-(2-Ethoxy-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J59)

The title compound was prepared from 3-(2-ethoxy-5-methylphenyl)-2-iminothiazolidin-4-one (prepared as in PCT International Application Publication WO 2017040194 A1) and was isolated as a yellow solid (70 mg, 76%).

### (Z)-1-(3-(2-(1-Methoxyethyl)-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J60)

The title compound was prepared from 2-imino-3-(2-(1-methoxyethyl)-5-methylphenyl)thiazolidin-4-one (prepared as in PCT International Application Publication WO 2017040194 A1) and was isolated as a yellow solid (37 mg, 39%).

### (Z)-1-(2-Fluoro-4-(1-(4-(trifluoromethyl)phenyl)-1H-1,2,4-triazol-3-yl)phenyl)-3-(3-(2-isopropyl-5-methylphenyl)-4-oxothiazolidin-2-ylidene)urea (J61)

The title compound was prepared from 2-imino-3-(2-isopropyl-5-methylphenyl)-thiazolidin-4-one (prepared as in U.S. Patent Application Publication 20140274688 A1) and was isolated as a pale orange solid (69 mg, 71%).

### (Z)-1-(2-Fluoro-4-(1-(4-(trifluoromethyl)phenyl)-1H-1,2,4-triazol-3-yl)phenyl)-3-(3-(2-(1-methoxyethyl)-5-methylphenyl)-4-oxothiazolidin-2-ylidene)urea (J62)

The title compound was prepared from 2-imino-3-(2-(1-methoxyethyl)-5-methylphenyl)thiazolidin-4-one (prepared as in PCT International Application Publication WO 2017040194 A1) and was isolated as an orange solid (58 mg, 60%).

### (Z)-1-(2-Fluoro-4-(1-(4-(trifluoromethyl)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J63)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white solid (81 mg, 78%).

### (Z)-1-(2-Fluoro-4-(1-(4-(trifluoromethyl)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J64)

The title compound was prepared from 2-imino-3-(5-methyl-2-(3,3,3-trifluoropropoxy)phenyl)thiazolidin-4-one (C99) and was isolated as an off-white solid (80 mg, 76%).

### (Z)-1-(3-(2-(sec-Butyl)-5-methylphenyl)-4-oxothiazolidin-2-ylidene)-3-(2-fluoro-4-(1-(4-(trifluoromethyl)phenyl)-1H-1,2,4-triazol-3-yl)phenyl)urea (J65)

The title compound was prepared from 3-(2-(sec-butyl)-5-methylphenyl)-2-iminothiazolidin-4-one (C115) and was isolated as an off-white solid (87 mg, 87%).

### (Z)-1-(2-Fluoro-4-(1-(4-(trifluoromethoxy)phenyl)-1H-1,2,4-triazol-3-yl)phenyl)-3-(3-(2-(methoxymethyl)-5-methylphenyl)-4-oxothiazolidin-2-ylidene)urea (J66)

The title compound was prepared from 2-imino-3-(2-(methoxymethyl)-5-methylphenyl)thiazolidin-4-one (prepared as in PCT International Application Publication WO 2017040742 A1) and was isolated as a white solid (37 mg, 20%).

### (Z)-1-(3-(2-(Methoxymethyl)-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J67)

The title compound was prepared from 2-imino-3-(2-(methoxymethyl)-5-methylphenyl)thiazolidin-4-one (prepared as in PCT International Application Publication WO 2017040742 A1) and was isolated as a white solid (130 mg, 70%).

### (Z)-1-(3-(5-(Dimethylamino)-2-isopropylphenyl)-4-oxothiazolidin-2-ylidene)-3-(2-methyl-4-(1-(4-(trifluoromethoxy)phenyl)-1H-1,2,4-triazol-3-yl)phenyl)urea (J68)

The title compound was prepared from 3-(5-(dimethylamino)-2-isopropylphenyl)-2-iminothiazolidin-4-one (C126) and was isolated as an orange solid (133 mg, 68%).

### (Z)-1-(2-Ethyl-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J70)

The title compound was prepared from 2-imino-3-(2-isopropyl-5-methylphenyl)thiazolidin-4-one (prepared as in U.S. Patent Application Publication 20140274688 A1) and was isolated as a yellow foam (37 mg, 41%).

### (Z)-1-(2-Ethyl-4-(1-(4-(trifluoromethoxy)phenyl)-1H-1,2,4-triazol-3-yl)phenyl)-3-(3-(2-isopropyl-5-methoxyphenyl)-4-oxothiazolidin-2-ylidene)urea (J71)

The title compound was prepared from 2-imino-3-(2-isopropyl-5-methoxyphenyl)thiazolidin-4-one (C98) and was isolated as a light yellow foam (46 mg, 48%).

### (Z)-1-(2-Ethyl-4-(1-(4-(trifluoromethoxy)phenyl)-1H-1,2,4-triazol-3-yl)phenyl)-3-(3-(2-(methoxymethyl)-5-methylphenyl)-4-oxothiazolidin-2-ylidene)urea (J72)

The title compound was prepared from 2-imino-3-(2-(methoxymethyl)-5-methylphenyl)thiazolidin-4-one (prepared as in PCT International Application Publication WO 2017040742 A1) and was isolated as a yellow foam (47 mg, 50%).

### (Z)-1-(2-Ethyl-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J73)

The title compound was prepared from 2-imino-3-(5-methyl-2-(3,3,3-trifluoropropoxy)phenyl)thiazolidin-4-one (C99) and was isolated as a yellow foam (45 mg, 43%).

### (Z)-1-(2-Ethyl-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J74)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow foam (31 mg, 32%).

### (Z)-1-(2-Ethyl-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J75)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as a yellow foam (62 mg, 61%).

### (Z)-1-(3-(5-Cyano-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J76)

The title compound was prepared from 3-(2-imino-4-oxothiazolidin-3-yl)-4-isopropylbenzonitrile (C108) and was isolated as a tan solid (59 mg, 28%).

### (Z)-1-(3-(5-Acetyl-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J77)

The title compound was prepared from 3-(5-acetyl-2-isopropylphenyl)-2-iminothiazolidin-4-one (C109) and was isolated as a white solid (31 mg, 32%).

### (Z)-1-(3-(5-(Dimethylamino)-2-isopropylphenyl)-4-oxothiazolidin-2-ylidene)-3-(2-ethyl-4-(1-(4-(trifluoromethoxy)phenyl)-1H-1,2,4-triazol-3-yl)phenyl)urea (J83)

The title compound was prepared from 3-(5-(dimethylamino)-2-isopropylphenyl)-2-iminothiazolidin-4-one (C126) and was isolated as a yellow foam (41 mg, 43%).

### (Z)-1-(3-(3-(Dimethylamino)phenyl)-4-oxothiazolidin-2-ylidene)-3-(2-fluoro-4-(1-(4-(trifluoromethoxy)phenyl)-1H-1,2,4-triazol-3-yl)phenyl)urea (J84)

The title compound was prepared from 3-(3-(dimethylamino)phenyl)-2-iminothiazolidin-4-one (C100) and was isolated as an off-white solid (34 mg, 38%).

### (Z)-1-(2-Fluoro-4-(1-(4-(trifluoromethoxy)phenyl)-1H-1,2,4-triazol-3-yl)phenyl)-3-(3-(2-isopropyl-5-(methylamino)phenyl)-4-oxothiazolidin-2-ylidene)urea (J85)

The title compound was prepared from 2-imino-3-(2-isopropyl-5-(methylamino)phenyl)thiazolidin-4-one hydrochloride (C150) and was isolated as an off-white solid (37 mg, 20%).

### (Z)-1-(2-Fluoro-4-(1-(4-(trifluoromethoxy)phenyl)-1H-1,2,4-triazol-3-yl)phenyl)-3-(3-(2-isopropylphenyl)-4-oxothiazolidin-2-ylidene)urea (J86)

The title compound was prepared from 2-imino-3-(2-isopropylphenyl)thiazolidin-4-one (prepared as in PCT International Application Publication WO 2016033025 A1) and was isolated as a white solid (27 mg, 30%).

### (Z)-1-(2-Fluoro-4-(1-(4-(trifluoromethoxy)phenyl)-1H-1,2,4-triazol-3-yl)phenyl)-3-(3-(2-(1-methoxyethyl)phenyl)-4-oxothiazolidin-2-ylidene)urea (J87)

The title compound was prepared from 2-imino-3-(2-(1-methoxyethyl)phenyl)thiazolidin-4-one (C124) and was isolated as a white foam (31 mg, 32%).

### (Z)-1-(2-Fluoro-4-(1-(4-(trifluoromethoxy)phenyl)-1H-1,2,4-triazol-3-yl)phenyl)-3-(3-(2-(1-(methylthio)ethyl)phenyl)-4-oxothiazolidin-2-ylidene)urea (J88)

The title compound was prepared from 2-imino-3-(2-(1-(methylthio)ethyl)phenyl)thiazolidin-4-one (C149) and was isolated as a light yellow solid (16 mg, 17%).

### (Z)-1-(3-(5-(Dimethylamino)-2-isopropylphenyl)-4-oxothiazolidin-2-ylidene)-3-(2-methoxy-4-(1-(4-(trifluoromethoxy)phenyl)-1H-1,2,4-triazol-3-yl)phenyl)urea (J89)

The title compound was prepared from 3-(5-(dimethylamino)-2-isopropylphenyl)-2-iminothiazolidin-4-one (C126) and was isolated as an off-white solid (62 mg, 65%).

### (Z)-1-(2-Methoxy-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J90)

The title compound was prepared from 2-imino-3-(2-(1-methoxyethyl)-5-methylphenyl)thiazolidin-4-one (prepared as in PCT International Application Publication WO 2017040194 A1) and was isolated as an off-white solid (56 mg, 60%).

### (Z)-1-(2-Methoxy-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J91)

The title compound was prepared from 2-imino-3-(5-methyl-2-(3,3,3-trifluoropropoxy)phenyl)thiazolidin-4-one (C99) and was isolated as an off-white foam (46 mg, 46%).

### (Z)-1-(2-Fluoro-4-(1-(4-(trifluoromethoxy)phenyl)-1H-1,2,4-triazol-3-yl)phenyl)-3-(3-(5-methoxy-2-(3,3,3-trifluoropropoxy)phenyl)-4-oxothiazolidin-2-ylidene)urea (J92)

The title compound was prepared from 2-imino-3-(5-methoxy-2-(3,3,3-trifluoropropoxy)phenyl)thiazolidin-4-one (C110) and was isolated as an off-white solid (68 mg, 65%).

### (Z)-1-(2-Isopropyl-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J93)

The title compound was prepared from 2-imino-3-(5-methyl-2-(3,3,3-trifluoropropoxy)phenyl)thiazolidin-4-one (C99) and was isolated as an off-white foam (147 mg, 74%).

### (Z)-1-(2-Isopropyl-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J94)

The title compound was prepared from 2-imino-3-(2-(1-methoxyethyl)-5-methylphenyl)thiazolidin-4-one (prepared as in PCT International Application Publication WO 2017040194 A1) and was isolated as a white fluffy solid (76 mg, 41%).

### (Z)-1-(2-Isopropyl-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J99)

The title compound was prepared from 2-imino-3-(2-isopropyl-5-methylphenyl)thiazolidin-4-one (prepared as in U.S. Patent Application Publication 20140274688 A1) and was isolated as an off-white solid (113 mg, 61%).

### (Z)-1-(2-Isopropyl-4-(1-(4-(trifluoromethoxy)phenyl)-1H-1,2,4-triazol-3-yl)phenyl)-3-(3-(2-isopropyl-5-methoxyphenyl)-4-oxothiazolidin-2-ylidene)urea (J100)

The title compound was prepared from 2-imino-3-(2-isopropyl-5-methoxyphenyl)thiazolidin-4-one (C98) and was isolated as a pale orange solid (125 mg, 66%).

### (Z)-1-(3-(5-(Dimethylamino)-2-isopropylphenyl)-4-oxothiazolidin-2-ylidene)-3-(2-isopropyl-4-(1-(4-(trifluoromethoxy)phenyl)-1H-1,2,4-triazol-3-yl)phenyl)urea (J101)

The title compound was prepared from 3-(5-(dimethylamino)-2-isopropylphenyl)-2-iminothiazolidin-4-one (C126) and was isolated as an off-white solid (127 mg, 66%).

### (Z)-1-(3-(5-(Dimethylamino)-2-(3,3,3-trifluoropropoxy)phenyl)-4-oxothiazolidin-2-ylidene)-3-(2-methyl-4-(1-(4-(trifluoromethoxy)phenyl)-1H-1,2,4-triazol-3-yl)phenyl)urea (J102)

The title compound was prepared from 3-(5-(dimethylamino)-2-(3,3,3-trifluoropropoxy)phenyl)-2-iminothiazolidin-4-one (C101) and was isolated as an off-white foam (89 mg, 82%).

### (Z)-1-(3-(5-Cyano-2-(3,3,3-trifluoropropoxy)phenyl)-4-oxothiazolidin-2-ylidene)-3-(2-fluoro-4-(1-(4-(trifluoromethoxy)phenyl)-1H-1,2,4-triazol-3-yl)phenyl)urea (J103)

The title compound was prepared from 3-(2-imino-4-oxothiazolidin-3-yl)-4-(3,3,3-trifluoropropoxy)benzonitrile (C111) and was isolated as a light yellow/orange solid (48 mg, 46%).

### Methyl (Z)-3-(2-(((2-fluoro-4-(1-(4-(trifluoromethoxy)phenyl)-1H-1,2,4-triazol-3-yl)phenyl)carbamoyl)imino)-4-oxothiazolidin-3-yl)-4-isopropylbenzoate (J104)

The title compound was prepared from methyl 3-(2-imino-4-oxothiazolidin-3-yl)-4-isopropylbenzoate (C112) and was isolated as a white solid (25 mg, 26%).

### (Z)-1-(3-(5-(Dimethylamino)-2-(3,3,3-trifluoropropoxy)phenyl)-4-oxothiazolidin-2-ylidene)-3-(2-fluoro-4-(1-(4-(trifluoromethoxy)phenyl)-1H-1,2,4-triazol-3-yl)phenyl)urea (J105)

The title compound was prepared from 3-(5-(dimethylamino)-2-(3,3,3-trifluoropropoxy)phenyl)-2-iminothiazolidin-4-one (C101) and was isolated as a white solid (67 mg, 61%).

### (Z)-1-(3-(5-(Dimethylamino)-2-(3,3,3-trifluoropropoxy)phenyl)-4-oxothiazolidin-2-ylidene)-3-(2-methoxy-4-(1-(4-(trifluoromethoxy)phenyl)-1H-1,2,4-triazol-3-yl)phenyl)urea (J109)

The title compound was prepared from 3-(5-(dimethylamino)-2-(3,3,3-trifluoropropoxy)phenyl)-2-iminothiazolidin-4-one (C101) and was isolated as a yellow solid (84 mg, 80%).

### (Z)-1-(3-(5-(Dimethylamino)-2-(3,3,3-trifluoropropoxy)phenyl)-4-oxothiazolidin-2-ylidene)-3-(2-ethyl-4-(1-(4-(trifluoromethoxy)phenyl)-1H-1,2,4-triazol-3-yl)phenyl)urea (J110)

The title compound was prepared from 3-(5-(dimethylamino)-2-(3,3,3-trifluoropropoxy)phenyl)-2-iminothiazolidin-4-one (C101) and was isolated as an off-white solid (47 mg, 45%).

### (Z)-1-(3-(5-(Dimethylamino)-2-(3,3,3-trifluoropropoxy)phenyl)-4-oxothiazolidin-2-ylidene)-3-(2-isopropyl-4-(1-(4-(trifluoromethoxy)phenyl)-1H-1,2,4-triazol-3-yl)phenyl)urea (J111)

The title compound was prepared from 3-(5-(dimethylamino)-2-(3,3,3-trifluoropropoxy)phenyl)-2-iminothiazolidin-4-one (C101) and was isolated as a yellow solid (82 mg, 79%).

### (Z)-1-(3-(4-(Dimethylamino)-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J112)

The title compound was prepared from 3-(4-(dimethylamino)-2-isopropylphenyl)-2-iminothiazolidin-4-one (C102) and was isolated as an off-white solid (62 mg, 64%).

### (Z)-1-(3-(4-(Dimethylamino)-2-isopropylphenyl)-4-oxothiazolidin-2-ylidene)-3-(2-methyl-4-(1-(4-(trifluoromethoxy)phenyl)-1H-1,2,4-triazol-3-yl)phenyl)urea (J113)

The title compound was prepared from 3-(4-(dimethylamino)-2-isopropylphenyl)-2-iminothiazolidin-4-one (C102) and was isolated as a light yellow foam (82 mg, 82%).

### (Z)-1-(2-Fluoro-4-(1-(3-(trifluoromethyl)phenyl)-1H-1,2,4-triazol-3-yl)phenyl)-3-(3-(2-(1-methoxyethyl)-5-methylphenyl)-4-oxothiazolidin-2-ylidene)urea (J119)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (75 mg, 47%).

### (Z)-1-(2-Fluoro-4-(1-(3-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J120)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (74 mg, 47%).

### (Z)-1-(2-Fluoro-4-(1-(3-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J121)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white solid (44 mg, 26%).

### (Z)-1-(2-Fluoro-4-(1-(3-(trifluoromethyl)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J122)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white solid (84 mg, 49%).

### (Z)-1-(2-Fluoro-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J123)

The title compound was prepared from 2-imino-3-(5-methyl-2-((2,2,2-trifluoroethoxy)methyl)phenyl)thiazolidin-4-one (C117) and was isolated as an off-white foam (173 mg, 84%).

### (Z)-1-(3-(2-(1-Ethoxyethyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J124)

The title compound was prepared from 3-(2-(1-ethoxyethyl)-5-methylphenyl)-2-iminothiazolidin-4-one (C118) and was isolated as an off-white solid (148 mg, 76%).

### (Z)-1-(2-Fluoro-4-(1-(4-(trifluoromethoxy)phenyl)-1H-1,2,4-triazol-3-yl)phenyl)-3-(3-(5-methyl-2-propoxyphenyl)-4-oxothiazolidin-2-ylidene)urea (J125)

The title compound was prepared from 2-imino-3-(5-methyl-2-propoxyphenyl)thiazolidin-4-one (C119) and was isolated as an off-white foam (166 mg, 88%).

### (Z)-1-(3-(2-Isopropyl-5-methylphenyl)-4-oxothiazolidin-2-ylidene)-3-(2-methyl-4-(1-(3-(trifluoromethyl)phenyl)-1H-1,2,4-triazol-3-yl)phenyl)urea (J131)

The title compound was prepared from 2-imino-3-(2-isopropyl-5-methylphenyl)thiazolidin-4-one (prepared as in U.S. Patent Application Publication 20140274688 A1) and was isolated as an off-white solid (62 mg, 41%).

### (Z)-1-(3-(2-Isopropyl-5-methoxyphenyl)-4-oxothiazolidin-2-ylidene)-3-(2-methyl-4-(1-(3-(trifluoromethyl)phenyl)-1H-1,2,4-triazol-3-yl)phenyl)urea (J132)

The title compound was prepared from 2-imino-3-(2-isopropyl-5-methoxyphenyl)thiazolidin-4-one (C98) and was isolated as an off-white solid (76 mg, 49%).

### (Z)-1-(3-(5-(Dimethylamino)-2-isopropylphenyl)-4-oxothiazolidin-2-ylidene)-3-(2-methyl-4-(1-(3-(trifluoromethyl)phenyl)-1H-1,2,4-triazol-3-yl)phenyl)urea (J133)

The title compound was prepared from 3-(5-(dimethylamino)-2-isopropylphenyl)-2-iminothiazolidin-4-one (C126) and was isolated as an off-white solid (69 mg, 43%).

### (Z)-1-(3-(5-Methyl-2-(3,3,3-trifluoropropoxy)phenyl)-4-oxothiazolidin-2-ylidene)-3-(2-methyl-4-(1-(3-(trifluoromethyl)phenyl)-1H-1,2,4-triazol-3-yl)phenyl)urea (J134)

The title compound was prepared from 2-imino-3-(5-methyl-2-(3,3,3-trifluoropropoxy)phenyl)thiazolidin-4-one (C99) and was isolated as an off-white solid (84 mg, 50%).

### (Z)-1-(3-(2-Isopropyl-5-methylphenyl)-4-oxothiazolidin-2-ylidene)-3-(2-methyl-4-(1-(3-(trifluoromethoxy)phenyl)-1H-1,2,4-triazol-3-yl)phenyl)urea (J135)

The title compound was prepared from 2-imino-3-(2-isopropyl-5-methylphenyl)-thiazolidin-4-one (prepared as in U.S. Patent Application Publication 20140274688 A1) and was isolated as an off-white solid (62 mg, 42%).

### (Z)-1-(3-(2-Isopropyl-5-methoxyphenyl)-4-oxothiazolidin-2-ylidene)-3-(2-methyl-4-(1-(3-(trifluoromethoxy)phenyl)-1H-1,2,4-triazol-3-yl)phenyl)urea (J136)

The title compound was prepared from 2-imino-3-(2-isopropyl-5-methoxyphenyl)thiazolidin-4-one (C98) and was isolated as an off-white solid (120 mg, 79%).

### (Z)-1-(3-(5-(Dimethylamino)-2-isopropylphenyl)-4-oxothiazolidin-2-ylidene)-3-(2-methyl-4-(1-(3-(trifluoromethoxy)phenyl)-1H-1,2,4-triazol-3-yl)phenyl)urea (J137)

The title compound was prepared from 3-(5-(dimethylamino)-2-isopropylphenyl)-2-iminothiazolidin-4-one (C126) and was isolated as an off-white solid (125 mg, 80%).

### (Z)-1-(3-(5-Methyl-2-(3,3,3-trifluoropropoxy)phenyl)-4-oxothiazolidin-2-ylidene)-3-(2-methyl-4-(1-(3-(trifluoromethoxy)phenyl)-1H-1,2,4-triazol-3-yl)phenyl)urea (J138)

The title compound was prepared from 2-imino-3-(5-methyl-2-(3,3,3-trifluoropropoxy)phenyl)thiazolidin-4-one (C99) and was isolated as an off-white solid (73 mg, 44%).

### (Z)-1-(3-(5-Methyl-2-((2,2,2-trifluoroethoxy)methyl)phenyl)-4-oxothiazolidin-2-ylidene)-3-(2-methyl-4-(1-(4-(trifluoromethoxy)phenyl)-1H-1,2,4-triazol-3-yl)phenyl)urea (J139)

The title compound was prepared from 2-imino-3-(5-methyl-2-((2,2,2-trifluoroethoxy)methyl)phenyl)thiazolidin-4-one (C117) and was isolated as an off-white solid (42 mg, 41%).

### (Z)-1-(2-Fluoro-4-(1-(3-(trifluoromethyl)phenyl)-1H-1,2,4-triazol-3-yl)phenyl)-3-(3-(2-isopropyl-5-methylphenyl)-4-oxothiazolidin-2-ylidene)urea (J140)

The title compound was prepared from 2-imino-3-(2-isopropyl-5-methylphenyl)-thiazolidin-4-one (prepared as in U.S. Patent Application Publication 20140274688 A1) and was isolated as an off-white solid (56 mg, 37%).

### (Z)-1-(3-(2-(1-Ethoxyethyl)-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J141)

The title compound was prepared from 3-(2-(1-ethoxyethyl)-5-methylphenyl)-2-iminothiazolidin-4-one (C118) and was isolated as an off-white solid (67 mg, 69%).

### (Z)-1-(2-Fluoro-4-(1-(3-(trifluoromethyl)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J142)

The title compound was prepared from 2-imino-3-(5-methyl-2-(3,3,3-trifluoropropoxy)phenyl)thiazolidin-4-one (C99) and was isolated as an off-white solid (60 mg, 36%).

### (Z)-1-(2-Fluoro-4-(1-(3-(trifluoromethoxy)phenyl)-1H-1,2,4-triazol-3-yl)phenyl)-3-(3-(2-isopropyl-5-methylphenyl)-4-oxothiazolidin-2-ylidene)urea (J143)

The title compound was prepared from 2-imino-3-(2-isopropyl-5-methylphenyl)-thiazolidin-4-one (prepared as in U.S. Patent Application Publication 20140274688 A1) and was isolated as a tan solid (94 mg, 63%).

### (Z)-1-(2-Fluoro-4-(1-(4-methoxyphenyl)-1H-1,2,4-triazol-3-yl)phenyl)-3-(3-(2-isopropyl-5-methylphenyl)-4-oxothiazolidin-2-ylidene)urea (J144)

The title compound was prepared from 2-imino-3-(2-isopropyl-5-methylphenyl)-thiazolidin-4-one (prepared as in U.S. Patent Application Publication 20140274688 A1) and was isolated as a tan solid (54 mg, 34%).

### (Z)-1-(2-Fluoro-4-(1-(4-methoxyphenyl)-1H-1,2,4-triazol-3-yl)phenyl)-3-(3-(2-isopropyl-5-methoxyphenyl)-4-oxothiazolidin-2-ylidene)urea (J145)

The title compound was prepared from 2-imino-3-(2-isopropyl-5-methoxyphenyl)thiazolidin-4-one (C98) and was isolated as an off-white solid (84 mg, 51%).

### (Z)-1-(3-(5-(Dimethylamino)-2-isopropylphenyl)-4-oxothiazolidin-2-ylidene)-3-(2-fluoro-4-(1-(4-methoxyphenyl)-1H-1,2,4-triazol-3-yl)phenyl)urea (J146)

The title compound was prepared from 3-(5-(dimethylamino)-2-isopropylphenyl)-2-iminothiazolidin-4-one (C126) and was isolated as an off-white solid (102 mg, 61%).

### (Z)-1-(2-Fluoro-4-(1-(4-methoxyphenyl)-1H-1,2,4-triazol-3-yl)phenyl)-3-(3-(5-methyl-2-(3,3,3-trifluoropropoxy)phenyl)-4-oxothiazolidin-2-ylidene)urea (J147)

The title compound was prepared from 2-imino-3-(5-methyl-2-(3,3,3-trifluoropropoxy)phenyl)thiazolidin-4-one (C99) and was isolated as a tan solid (77 mg, 43%).

### (Z)-1-(2-Fluoro-4-(1-(p-tolyl)-1H-1,2,4-triazol-3-yl)phenyl)-3-(3-(2-isopropyl-5-methylphenyl)-4-oxothiazolidin-2-ylidene)urea (J148)

The title compound was prepared from 2-imino-3-(2-isopropyl-5-methylphenyl)-thiazolidin-4-one (prepared as in U.S. Patent Application Publication 20140274688 A1) and was isolated as an off-white solid (73 mg, 44%).

### (Z)-1-(2-Fluoro-4-(1-(p-tolyl)-1H-1,2,4-triazol-3-yl)phenyl)-3-(3-(2-isopropyl-5-methoxyphenyl)-4-oxothiazolidin-2-ylidene)urea (J149)

The title compound was prepared from 2-imino-3-(2-isopropyl-5-methoxyphenyl)thiazolidin-4-one (C98) and was isolated as an off-white solid (74 mg, 44%).

### (Z)-1-(3-(5-(Dimethylamino)-2-isopropylphenyl)-4-oxothiazolidin-2-ylidene)-3-(2-fluoro-4-(1-(p-tolyl)-1H-1,2,4-triazol-3-yl)phenyl)urea (J150)

The title compound was prepared from 3-(5-(dimethylamino)-2-isopropylphenyl)-2-iminothiazolidin-4-one (C126) and was isolated as an off-white solid (51 mg, 29%).

### (Z)-1-(2-Fluoro-4-(1-(p-tolyl)-1H-1,2,4-triazol-3-yl)phenyl)-3-(3-(5-methyl-2-(3,3,3-trifluoropropoxy)phenyl)-4-oxothiazolidin-2-ylidene)urea (J151)

The title compound was prepared from 2-imino-3-(5-methyl-2-(3,3,3-trifluoropropoxy)phenyl)thiazolidin-4-one (C99) and was isolated as a tan solid (85 mg, 46%).

### (Z)-1-(2-Fluoro-4-(1-(3-(trifluoromethyl)phenyl)-1H-1,2,4-triazol-3-yl)phenyl)-3-(3-(2-isopropyl-5-methoxyphenyl)-4-oxothiazolidin-2-ylidene)urea (J152)

The title compound was prepared from 2-imino-3-(2-isopropyl-5-methoxyphenyl)-thiazolidin-4-one (C98) and was isolated as an off-white solid (84 mg, 54%).

### (Z)-1-(3-(5-(Dimethylamino)-2-isopropylphenyl)-4-oxothiazolidin-2-ylidene)-3-(2-fluoro-4-(1-(3-(trifluoromethyl)phenyl)-1H-1,2,4-triazol-3-yl)phenyl)urea (J153)

The title compound was prepared from 3-(5-(dimethylamino)-2-isopropylphenyl)-2-iminothiazolidin-4-one (C126) and was isolated as an off-white solid (95 mg, 60%).

### (Z)-1-(2-Fluoro-4-(1-(3-(trifluoromethoxy)phenyl)-1H-1,2,4-triazol-3-yl)phenyl)-3-(3-(2-isopropyl-5-methoxyphenyl)-4-oxothiazolidin-2-ylidene)urea (J154)

The title compound was prepared from 2-imino-3-(2-isopropyl-5-methoxyphenyl)-thiazolidin-4-one (C98) and was isolated as an off-white solid (74 mg, 49%).

### (Z)-1-(3-(5-(Dimethylamino)-2-isopropylphenyl)-4-oxothiazolidin-2-ylidene)-3-(2-fluoro-4-(1-(3-(trifluoromethoxy)phenyl)-1H-1,2,4-triazol-3-yl)phenyl)urea (J155)

The title compound was prepared from 3-(5-(dimethylamino)-2-isopropylphenyl)-2-iminothiazolidin-4-one (C126) and was isolated as an off-white solid (100 mg, 64%).

### (Z)-1-(2-Fluoro-4-(1-(3-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J156)

The title compound was prepared from 2-imino-3-(5-methyl-2-(3,3,3-trifluoropropoxy)phenyl)thiazolidin-4-one (C99) and was isolated as a white solid (40 mg, 24%).

### (Z)-1-(3-(5-(Dimethylamino)-4-fluoro-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J162)

The title compound was prepared from 3-(5-(dimethylamino)-4-fluoro-2-isopropylphenyl)-2-iminothiazolidin-4-one (C103) and was isolated as an off-white solid (140 mg, 70%).

### (Z)-1-(3-(2,5-Diisopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J164)

The title compound was prepared from 3-(2,5-diisopropylphenyl)-2-iminothiazolidin-4-one (C104) and was isolated as a white solid (68 mg, 68%).

### (Z)-1-(2-Chloro-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J166)

The title compound was prepared from 3-(5-(dimethylamino)-2-isopropylphenyl)-2-iminothiazolidin-4-one (C126) and was isolated as an off-white solid (26 mg, 27%).

### (Z)-1-(3-(5-Methyl-2-((2,2,2-trifluoroethoxy)methyl)phenyl)-4-oxothiazolidin-2-ylidene)-3-(2-methyl-4-(1-(4-(trifluoromethyl)phenyl)-1H-1,2,4-triazol-3-yl)phenyl)urea (J167)

The title compound was prepared from 2-imino-3-(5-methyl-2-((2,2,2-trifluoroethoxy)methyl)phenyl)thiazolidin-4-one (C117) and was isolated as an off-white solid (73 mg, 69%).

### (Z)-1-(2-Fluoro-4-(1-(4-(trifluoromethyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J168)

The title compound was prepared from 2-imino-3-(5-methyl-2-((2,2,2-trifluoroethoxy)methyl)phenyl)thiazolidin-4-one (C117) and was isolated as a white foam (65 mg, 62%).

### (Z)-1-(3-(5-Ethoxy-2-propylphenyl)-4-oxothiazolidin-2-ylidene)-3-(2-fluoro-4-(1-(4-(trifluoromethoxy)phenyl)-1H-1,2,4-triazol-3-yl)phenyl)urea (J169)

The title compound was prepared from 3-(5-ethoxy-2-propylphenyl)-2-iminothiazolidin-4-one (C125) and was isolated as a light yellow-orange solid (81 mg, 81%).

### (Z)-1-(3-(2-(Ethoxymethyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J180)

The title compound was prepared from 3-(2-(ethoxymethyl)-5-methylphenyl)-2-iminothiazolidin-4-one (C120) and was isolated as a yellow foam (54 mg, 57%).

### (Z)-1-(2-Fluoro-4-(1-(4-(trifluoromethoxy)phenyl)-1H-1,2,4-triazol-3-yl)phenyl)-3-(3-(5-methyl-2-(2,2,2-trifluoro-1-methoxyethyl)phenyl)-4-oxothiazolidin-2-ylidene)urea (J181)

The title compound was prepared from 2-imino-3-(5-methyl-2-(2,2,2-trifluoro-1-methoxyethyl)phenyl)thiazolidin-4-one (C121) and was isolated as an off-white solid (101 mg, 98%).

### (Z)-1-(3-(5-(Dimethylamino)-2-(trifluoromethyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J182)

The title compound was prepared from 3-(5-(dimethylamino)-2-(trifluoromethyl)phenyl)-2-iminothiazolidin-4-one (C127) and was isolated as an off-white solid (76 mg, 75%).

### (Z)-1-(3-(5-(Dimethylamino)-4-fluoro-2-isopropylphenyl)-4-oxothiazolidin-2-ylidene)-3-(2-fluoro-4-(1-(4-(trifluoromethyl)phenyl)-1H-1,2,4-triazol-3-yl)phenyl)urea (J183)

The title compound was prepared from 3-(5-(dimethylamino)-4-fluoro-2-isopropylphenyl)-2-iminothiazolidin-4-one (C103) and was isolated as an off-white solid (58 mg, 57%).

### (Z)-1-(3-(5-(Dimethylamino)-2-(3,3,3-trifluoropropoxy)phenyl)-4-oxothiazolidin-2-ylidene)-3-(2-fluoro-4-(1-(4-(trifluoromethyl)phenyl)-1H-1,2,4-triazol-3-yl)phenyl)urea (J184)

The title compound was prepared from 3-(5-(dimethylamino)-2-(3,3,3-trifluoropropoxy)phenyl)-2-iminothiazolidin-4-one (C101) and was isolated as an off-white solid (69 mg, 63%).

### (Z)-1-(2-Fluoro-4-(1-(4-(trifluoromethoxy)phenyl)-1H-1,2,4-triazol-3-yl)phenyl)-3-(3-(5-methoxy-2-(trifluoromethyl)phenyl)-4-oxothiazolidin-2-ylidene)urea (J185)

The title compound was prepared from 2-imino-3-(5-methoxy-2-(trifluoromethyl)phenyl)thiazolidin-4-one (C140) and was isolated as a white oily solid (37 mg, 31%).

### (Z)-1-(2-Fluoro-4-(1-(4-((trifluoromethyl)thio)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J186)

The title compound was prepared from 2-imino-3-(5-methyl-2-((2,2,2-trifluoroethoxy)methyl)phenyl)thiazolidin-4-one (C117) and was isolated as a yellow foam (88 mg, 85%).

### (Z)-1-(2-Fluoro-4-(1-(4-((trifluoromethyl)thio)phenyl)-1H-1,2,4-triazol-3-yl)phenyl)-3-(3-(2-(1-methoxyethyl)-5-methylphenyl)-4-oxothiazolidin-2-ylidene)urea (J187)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white foam (71 mg, 74%).

### (Z)-1-(3-(5-Ethoxy-2-(trifluoromethyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J190)

The title compound was prepared from 3-(5-ethoxy-2-(trifluoromethyl)phenyl)-2-iminothiazolidin-4-one (C141) and was isolated as a tan solid (56 mg, 45%).

### (Z)-1-(3-(5-Ethoxy-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J191)

The title compound was prepared from 3-(5-ethoxy-2-isopropylphenyl)-2-iminothiazolidin-4-one (C147) and was isolated as a tan solid (76 mg, 60%).

### (Z)-1-(3-(5-(Dimethylamino)-2-ethoxyphenyl)-4-oxothiazolidin-2-ylidene)-3-(2-fluoro-4-(1-(4-(trifluoromethoxy)phenyl)-1H-1,2,4-triazol-3-yl)phenyl)urea (J192)

The title compound was prepared from 3-(5-ethoxy-2-isopropylphenyl)-2-iminothiazolidin-4-one (C147) and was isolated as a light orange solid (57 mg, 59%).

### (Z)-1-(3-(5-(Dimethylamino)-2-(2,2,2-trifluoroethoxy)phenyl)-4-oxothiazolidin-2-ylidene)-3-(2-fluoro-4-(1-(4-(trifluoromethoxy)phenyl)-1H-1,2,4-triazol-3-yl)phenyl)urea (J193)

The title compound was prepared from 3-(5-(dimethylamino)-2-(2,2,2-trifluoroethoxy)phenyl)-2-iminothiazolidin-4-one (C129) and was isolated as a light orange solid (60 mg, 57%).

### (Z)-1-(2-Chloro-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J194)

The title compound was prepared from 2-imino-3-(5-methyl-2-((2,2,2-trifluoroethoxy)methyl)phenyl)thiazolidin-4-one (C117) and was isolated as a white foam (27 mg, 27%).

### (Z)-1-(3-(2-(Ethoxymethyl)-5-methylphenyl)-4-oxothiazolidin-2-ylidene)-3-(2-fluoro-4-(1-(4-((trifluoromethyl)thio)phenyl)-1H-1,2,4-triazol-3-yl)phenyl)urea (J195)

The title compound was prepared from 3-(2-(ethoxymethyl)-5-methylphenyl)-2-iminothiazolidin-4-one (C120) and was isolated as a yellow foam (40 mg, 43%).

### (Z)-1-(3-(5-(Dimethylamino)-2-propylphenyl)-4-oxothiazolidin-2-ylidene)-3-(2-fluoro-4-(1-(4-(trifluoromethoxy)phenyl)-1H-1,2,4-triazol-3-yl)phenyl)urea (J196)

The title compound was prepared from 3-(5-(dimethylamino)-2-propylphenyl)-2-iminothiazolidin-4-one (C128) and was isolated as a light orange solid (65 mg, 67%).

### (Z)-1-(2-Fluoro-4-(1-(4-(trifluoromethoxy)phenyl)-1H-1,2,4-triazol-3-yl)phenyl)-3-(3-(5-hydroxy-2-isopropylphenyl)-4-oxothiazolidin-2-ylidene)urea (J197)

The title compound was prepared from 3-(5-hydroxy-2-isopropylphenyl)-2-iminothiazolidin-4-one (C105) and was isolated as a light yellow-orange solid (6 mg, 6%).

### (Z)-1-(2-Fluoro-4-(1-(4-(trifluoromethoxy)phenyl)-1H-1,2,4-triazol-3-yl)phenyl)-3-(4-oxo-3-(2-(piperidin-1-yl)phenyl)thiazolidin-2-ylidene)urea (J198)

The title compound was prepared from 2-imino-3-(2-(piperidin-1-yl)phenyl)thiazolidin-4-one (C106) and was isolated as a light yellow-orange solid (34 mg, 35%).

### (Z)-1-(2-Fluoro-4-(1-(4-(trifluoromethoxy)phenyl)-1H-1,2,4-triazol-3-yl)phenyl)-3-(3-(5-methoxy-2-(2,2,2-trifluoroethoxy)phenyl)-4-oxothiazolidin-2-ylidene)urea (J201)

The title compound was prepared from 2-imino-3-(5-methoxy-2-(2,2,2-trifluoroethoxy)phenyl)thiazolidin-4-one (C142) and was isolated as an off-white solid (30 mg, 25%).

### (Z)-1-(3-(4-Fluoro-2-isopropyl-5-methoxyphenyl)-4-oxothiazolidin-2-ylidene)-3-(2-fluoro-4-(1-(4-(trifluoromethoxy)phenyl)-1H-1,2,4-triazol-3-yl)phenyl)urea (J202)

The title compound was prepared from 3-(4-fluoro-2-isopropyl-5-methoxyphenyl)-2-iminothiazolidin-4-one (C143) and was isolated as a white solid (30 mg, 25%).

### (Z)-1-(2-Fluoro-4-(1-(4-(trifluoromethoxy)phenyl)-1H-1,2,4-triazol-3-yl)phenyl)-3-(3-(2-(1-methoxyethyl)-4-methylphenyl)-4-oxothiazolidin-2-ylidene)urea (J203)

The title compound was prepared from 2-imino-3-(2-(1-methoxyethyl)-4-methylphenyl)thiazolidin-4-one (C144) and was isolated as a tan oily foam (35 mg, 29%).

### (Z)-1-(3-(5-Methyl-2-((2,2,2-trifluoroethoxy)methyl)phenyl)-4-oxothiazolidin-2-ylidene)-3-(2-methyl-4-(1-(4-((trifluoromethyl)thio)phenyl)-1H-1,2,4-triazol-3-yl)phenyl)urea (J204)

The title compound was prepared from 2-imino-3-(5-methyl-2-((2,2,2-trifluoroethoxy)methyl)phenyl)thiazolidin-4-one (C117) and was isolated as an off-white solid (56 mg, 55%).

### (Z)-1-(3-(2-(Ethoxymethyl)-5-methylphenyl)-4-oxothiazolidin-2-ylidene)-3-(2-methyl-4-(1-(4-((trifluoromethyl)thio)phenyl)-1H-1,2,4-triazol-3-yl)phenyl)urea (J205)

The title compound was prepared from 3-(2-(ethoxymethyl)-5-methylphenyl)-2-iminothiazolidin-4-one (C120) and was isolated as a yellow foam (60 mg, 64%).

### (Z)-1-(3-(2-(Ethoxymethyl)-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J206)

The title compound was prepared from 3-(2-(ethoxymethyl)-5-methylphenyl)-2-iminothiazolidin-4-one (C120) and was isolated as an orange foam (124 mg, 65%).

### (Z)-1-(3-(2-Ethyl-5-methoxyphenyl)-4-oxothiazolidin-2-ylidene)-3-(2-fluoro-4-(1-(4-(trifluoromethoxy)phenyl)-1H-1,2,4-triazol-3-yl)phenyl)urea (J216)

The title compound was prepared from 3-(2-ethyl-5-methoxyphenyl)-2-iminothiazolidin-4-one (C148) and was isolated as an off-white solid (27 mg, 29%).

### (Z)-1-(2-Fluoro-4-(1-(4-((trifluoromethyl)thio)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J223)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as a white solid (45 mg, 46%).

### (Z)-1-(3-(2-Ethoxy-5-methylphenyl)-4-oxothiazolidin-2-ylidene)-3-(2-fluoro-4-(1-(4-((trifluoromethyl)thio)phenyl)-1H-1,2,4-triazol-3-yl)phenyl)urea (J224)

The title compound was prepared from 3-(2-ethoxy-5-methylphenyl)-2-iminothiazolidin-4-one prepared as in PCT International Application Publication WO 2017040194 A1) and was isolated as a white solid (51 mg, 54%).

### (Z)-1-(2-Fluoro-4-(1-(4-((trifluoromethyl)thio)phenyl)-1H-1,2,4-triazol-3-yl)phenyl)-3-(3-(5-methyl-2-(4,4,4-trifluorobutoxy)phenyl)-4-oxothiazolidin-2-ylidene)urea (J225)

The title compound was prepared from 2-imino-3-(5-methyl-2-(4,4,4-trifluorobutoxy)phenyl)thiazolidin-4-one (C107) and was isolated as an off-white solid (52 mg, 51%).

### (Z)-1-(2-Chloro-4-(1-(4-((trifluoromethyl)thio)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J226)

The title compound was prepared from 2-imino-3-(5-methyl-2-((2,2,2-trifluoroethoxy)methyl)phenyl)thiazolidin-4-one (C117) and was isolated as a white solid (40 mg, 41%).

### (Z)-1-(2-Fluoro-4-(1-(4-((trifluoromethyl)thio)phenyl)-1H-1,2,4-triazol-3-yl)phenyl)-3-(3-(5-methyl-2-(2,2,2-trifluoro-1-methoxyethyl)phenyl)-4-oxothiazolidin-2-ylidene)urea (J229)

The title compound was prepared from 2-imino-3-(5-methyl-2-(2,2,2-trifluoro-1-methoxyethyl)phenyl)thiazolidin-4-one (C121) and was isolated as an off-white solid (46 mg, 44%).

### (Z)-1-(3-(2-(1-Ethoxyethyl)-5-methylphenyl)-4-oxothiazolidin-2-ylidene)-3-(2-fluoro-4-(1-(4-((trifluoromethyl)thio)phenyl)-1H-1,2,4-triazol-3-yl)phenyl)urea (J230)

The title compound was prepared from 3-(2-(1-ethoxyethyl)-5-methylphenyl)-2-iminothiazolidin-4-one (C118) and was isolated as an off white solid (70 mg, 72%).

### (Z)-1-(2-Chloro-4-(1-(4-((trifluoromethyl)thio)phenyl)-1H-1,2,4-triazol-3-yl)phenyl)-3-(3-(2-isopropyl-5-methylphenyl)-4-oxothiazolidin-2-ylidene)urea (J231)

The title compound was prepared from 2-imino-3-(2-isopropyl-5-methylphenyl)thiazolidin-4-one (prepared as in U.S. Patent Application Publication 20140274688 A1) and was isolated as a yellow solid (48 mg, 52%).

### (Z)-1-(2-Chloro-4-(1-(4-((trifluoromethyl)thio)phenyl)-1H-1,2,4-triazol-3-yl)phenyl)-3-(3-(2-(1-methoxyethyl)-5-methylphenyl)-4-oxothiazolidin-2-ylidene)urea (J232)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow solid (64 mg, 68%).

### (Z)-1-(3-(2-(1-Methoxyethyl)-5-methylphenyl)-4-oxothiazolidin-2-ylidene)-3-(2-methyl-4-(1-(4-((trifluoromethyl)thio)phenyl)-1H-1,2,4-triazol-3-yl)phenyl)urea (J233)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (58 mg, 60%).

### (Z)-1-(2-Chloro-4-(1-(4-(trifluoromethyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J234)

The title compound was prepared from 2-imino-3-(5-methyl-2-((2,2,2-trifluoroethoxy)methyl)phenyl)thiazolidin-4-one (C117) and was isolated as a white solid (36 mg, 35%).

### (Z)-1-(2-Chloro-4-(1-(4-(trifluoromethyl)phenyl)-1H-1,2,4-triazol-3-yl)phenyl)-3-(3-(5-(dimethylamino)-2-propylphenyl)-4-oxothiazolidin-2-ylidene)urea (J235)

The title compound was prepared from 3-(5-(dimethylamino)-2-propylphenyl)-2-iminothiazolidin-4-one (C128) and was isolated as a white solid (32 mg, 33%).

### (Z)-1-(2-Fluoro-4-(1-(5-(trifluoromethoxy)pyridin-2-yl)-1H-1,2,4-triazol-3-yl)phenyl)-3-(3-(2-(1-methoxyethyl)-5-methylphenyl)-4-oxothiazolidin-2-ylidene)urea (J236)

The title compound was prepared from 2-imino-3-(2-(1-methoxyethyl)-5-methylphenyl)-thiazolidin-4-one (prepared as in PCT International Application Publication WO 2017040194 A1) and was isolated as a white solid (47 mg, 50%).

### (Z)-1-(2-Fluoro-4-(1-(5-(trifluoromethoxy)pyridin-2-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J237)

The title compound was prepared from 2-imino-3-(5-methyl-2-((2,2,2-trifluoroethoxy)methyl)phenyl)thiazolidin-4-one (C117) and was isolated as an off-white solid (87 mg, 85%).

### (Z)-1-(3-(5-(Dimethylamino)-2-isopropylphenyl)-4-oxothiazolidin-2-ylidene)-3-(2-fluoro-4-(1-(5-(trifluoromethoxy)pyridin-2-yl)-1H-1,2,4-triazol-3-yl)phenyl)urea (J238)

The title compound was prepared from 3-(5-(dimethylamino)-2-isopropylphenyl)-2-iminothiazolidin-4-one (C126) and was isolated as an off-white solid (79 mg, 82%).

### (Z)-1-(2-Fluoro-4-(1-(5-(trifluoromethoxy)pyridin-2-yl)-1H-1,2,4-triazol-3-yl)phenyl)-3-(3-(2-isopropyl-5-methoxyphenyl)-4-oxothiazolidin-2-ylidene)urea (J239)

The title compound was prepared from 2-imino-3-(2-isopropyl-5-methoxyphenyl)thiazolidin-4-one (C98) and was isolated as an off-white solid (65 mg, 69%).

### (Z)-1-(3-(2-(1-Methoxyethyl)-5-methylphenyl)-4-oxothiazolidin-2-ylidene)-3-(2-methyl-4-(1-(5-(trifluoromethoxy)pyridin-2-yl)-1H-1,2,4-triazol-3-yl)phenyl)urea (J240)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (52 mg, 55%).

### (Z)-1-(3-(5-Methyl-2-((2,2,2-trifluoroethoxy)methyl)phenyl)-4-oxothiazolidin-2-ylidene)-3-(2-methyl-4-(1-(5-(trifluoromethoxy)pyridin-2-yl)-1H-1,2,4-triazol-3-yl)phenyl)urea (J241)

The title compound was prepared from 2-imino-3-(5-methyl-2-((2,2,2-trifluoroethoxy)methyl)phenyl)thiazolidin-4-one (C117) and was isolated as an off-white solid (78 mg, 75%).

### (Z)-1-(2-Chloro-4-(1-(5-(trifluoromethoxy)pyridin-2-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J249)

The title compound was prepared from 2-imino-3-(5-methyl-2-((2,2,2-trifluoroethoxy)methyl)phenyl)thiazolidin-4-one (C117) and was isolated as an off white solid (41 mg, 41%).

### (Z)-1-(2-Chloro-4-(1-(5-(trifluoromethoxy)pyridin-2-yl)-1H-1,2,4-triazol-3-yl)phenyl)-3-(3-(2-(1-methoxyethyl)-5-methylphenyl)-4-oxothiazolidin-2-ylidene)urea (J250)

The title compound was prepared from 2-imino-3-(2-(1-methoxyethyl)-5-methylphenyl)thiazolidin-4-one (prepared as in PCT International Application Publication WO 2017040194 A1) and was isolated as an off-white solid (30 mg, 32%).

### (Z)-1-(3-(5-(Dimethylamino)-2-isopropylphenyl)-4-oxothiazolidin-2-ylidene)-3-(2-methyl-4-(1-(5-(trifluoromethoxy)pyridin-2-yl)-1H-1,2,4-triazol-3-yl)phenyl)urea (J251)

The title compound was prepared from 3-(5-(dimethylamino)-2-isopropylphenyl)-2-iminothiazolidin-4-one (C126) and was isolated as an off-white solid (55 mg, 57%).

### (Z)-1-(3-(2-Isopropyl-5-methoxyphenyl)-4-oxothiazolidin-2-ylidene)-3-(2-methyl-4-(1-(5-(trifluoromethoxy)pyridin-2-yl)-1H-1,2,4-triazol-3-yl)phenyl)urea (J252)

The title compound was prepared from 2-imino-3-(2-isopropyl-5-methoxyphenyl)thiazolidin-4-one (C98) and was isolated as an off-white solid (59 mg, 62%).

### (Z)-1-(2-Chloro-4-(1-(5-(trifluoromethoxy)pyridin-2-yl)-1H-1,2,4-triazol-3-yl)phenyl)-3-(3-(5-(dimethylamino)-2-isopropylphenyl)-4-oxothiazolidin-2-ylidene)urea (J253)

The title compound was prepared from 3-(5-(dimethylamino)-2-isopropylphenyl)-2-iminothiazolidin-4-one (C126) and was isolated as an off-white solid (51 mg, 54%).

### (Z)-1-(3-(5-(Dimethylamino)-2-propylphenyl)-4-oxothiazolidin-2-ylidene)-3-(2-methyl-4-(1-(5-(trifluoromethoxy)pyridin-2-yl)-1H-1,2,4-triazol-3-yl)phenyl)urea (J254)

The title compound was prepared from 3-(5-(dimethylamino)-2-propylphenyl)-2-iminothiazolidin-4-one (C128) and was isolated as an off-white solid (60 mg, 62%).

### (Z)-1-(3-(5-Methyl-2-(3,3,3-trifluoropropoxy)phenyl)-4-oxothiazolidin-2-ylidene)-3-(2-methyl-4-(1-(5-(trifluoromethoxy)pyridin-2-yl)-1H-1,2,4-triazol-3-yl)phenyl)urea (J255)

The title compound was prepared from 2-imino-3-(5-methyl-2-(3,3,3-trifluoropropoxy)phenyl)thiazolidin-4-one (C99) and was isolated as an off-white solid (82 mg, 79%).

### (Z)-1-(2-Fluoro-4-(1-(4-(trifluoromethoxy)phenyl)-1H-1,2,4-triazol-3-yl)phenyl)-3-(3-(5-methoxy-2-(1-methoxyethyl)phenyl)-4-oxothiazolidin-2-ylidene)urea (J256)

The title compound was prepared from 2-imino-3-(5-methoxy-2-(1-methoxyethyl)phenyl)thiazolidin-4-one (C145) and was isolated as a tan solid (30 mg, 31%).

### (Z)-1-(2-Fluoro-4-(1-(5-(trifluoromethoxy)pyridin-2-yl)-1H-1,2,4-triazol-3-yl)phenyl)-3-(3-(5-methyl-2-(3,3,3-trifluoropropoxy)phenyl)-4-oxothiazolidin-2-ylidene)urea (J257)

The title compound was prepared from 2-imino-3-(5-methyl-2-(3,3,3-trifluoropropoxy)phenyl)thiazolidin-4-one (C99) and was isolated as an off-white solid (79 mg, 77%).

### (Z)-1-(3-(5-(Dimethylamino)-2-propylphenyl)-4-oxothiazolidin-2-ylidene)-3-(2-fluoro-4-(1-(5-(trifluoromethoxy)pyridin-2-yl)-1H-1,2,4-triazol-3-yl)phenyl)urea (J258)

The title compound was prepared from 3-(5-(dimethylamino)-2-propylphenyl)-2-iminothiazolidin-4-one (C128) and was isolated as an off-white solid (80 mg, 83%).

### (Z)-1-(3-(5-(Dimethylamino)-4-fluoro-2-isopropylphenyl)-4-oxothiazolidin-2-ylidene)-3-(2-fluoro-4-(1-(5-(trifluoromethoxy)pyridin-2-yl)-1H-1,2,4-triazol-3-yl)phenyl)urea (J261)

The title compound was prepared from 3-(5-(dimethylamino)-4-fluoro-2-isopropylphenyl)-2-iminothiazolidin-4-one (C103) and was isolated as an off-white solid (70 mg, 71%).

### (Z)-1-(3-(5-(Dimethylamino)-2-(trifluoromethyl)phenyl)-4-oxothiazolidin-2-ylidene)-3-(2-fluoro-4-(1-(5-(trifluoromethoxy)pyridin-2-yl)-1H-1,2,4-triazol-3-yl)phenyl)urea (J262)

The title compound was prepared from 3-(5-(dimethylamino)-2-(trifluoromethyl)phenyl)-2-iminothiazolidin-4-one (C127) and was isolated as an off-white solid (63 mg, 63%).

### (Z)-1-(3-(5-(Dimethylamino)-2-(2,2,2-trifluoroethoxy)phenyl)-4-oxothiazolidin-2-ylidene)-3-(2-fluoro-4-(1-(5-(trifluoromethoxy)pyridin-2-yl)-1H-1,2,4-triazol-3-yl)phenyl)urea (J263)

The title compound was prepared from 3-(5-(dimethylamino)-2-(2,2,2-trifluoroethoxy)phenyl)-2-iminothiazolidin-4-one (C129) and was isolated as an off-white solid (56 mg, 53%).

### (Z)-1-(2-Fluoro-4-(1-(5-(trifluoromethoxy)pyridin-2-yl)-1H-1,2,4-triazol-3-yl)phenyl)-3-(3-(2-isopropyl-5-methylphenyl)-4-oxothiazolidin-2-ylidene)urea (J270)

The title compound was prepared from 2-imino-3-(2-isopropyl-5-methylphenyl)thiazolidin-4-one (prepared as in U.S. Patent Application Publication 20140274688 A1) and was isolated as an off-white solid (49 mg, 53%).

### (Z)-1-(2-Fluoro-4-(1-(5-(trifluoromethoxy)pyridin-2-yl)-1H-1,2,4-triazol-3-yl)phenyl)-3-(3-(5-methyl-2-(trifluoromethyl)phenyl)-4-oxothiazolidin-2-ylidene)urea (J271)

The title compound was prepared from 2-imino-3-(5-methyl-2-(trifluoromethyl)phenyl)thiazolidin-4-one (prepared as in PCT International Application Publication WO 2017040742 A1) and was isolated as an off-white solid (54 mg, 56%).

### (Z)-1-(2-Fluoro-4-(1-(5-(trifluoromethoxy)pyridin-2-yl)-1H-1,2,4-triazol-3-yl)phenyl)-3-(3-(5-methyl-2-propylphenyl)-4-oxothiazolidin-2-ylidene)urea (J272)

The title compound was prepared from 2-imino-3-(5-methyl-2-propylphenyl)thiazolidin-4-one (prepared as in PCT International Application Publication WO 2016033025 A1) and was isolated as an off-white solid (33 mg, 36%).

### (Z)-1-(2-Fluoro-4-(1-(5-(trifluoromethyl)pyridin-2-yl)-1H-1,2,4-triazol-3-yl)phenyl)-3-(3-(2-isopropyl-5-methylphenyl)-4-oxothiazolidin-2-ylidene)urea (J279)

The title compound was prepared from 2-imino-3-(2-isopropyl-5-methylphenyl)thiazolidin-4-one (prepared as in U.S. Patent Application Publication 20140274688 A1) and was isolated as an off-white solid (47 mg, 50%).

### (Z)-1-(2-Fluoro-4-(1-(5-(trifluoromethyl)pyridin-2-yl)-1H-1,2,4-triazol-3-yl)phenyl)-3-(3-(2-(1-methoxyethyl)-5-methylphenyl)-4-oxothiazolidin-2-ylidene)urea (J280)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (54 mg, 56%).

### (Z)-1-(2-Fluoro-4-(1-(5-(trifluoromethyl)pyridin-2-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J281)

The title compound was prepared from 2-imino-3-(5-methyl-2-((2,2,2-trifluoroethoxy)methyl)phenyl)thiazolidin-4-one (C117) and was isolated as an off-white solid (67 mg, 64%).

### (Z)-1-(3-(5-(Dimethylamino)-2-propylphenyl)-4-oxothiazolidin-2-ylidene)-3-(2-fluoro-4-(1-(5-(trifluoromethyl)pyridin-2-yl)-1H-1,2,4-triazol-3-yl)phenyl)urea (J282)

The title compound was prepared from 3-(5-(dimethylamino)-2-propylphenyl)-2-iminothiazolidin-4-one (C128) and was isolated as an off-white solid (66 mg, 67%).

### (Z)-1-(2-fluoro-4-(1-(5-(trifluoromethyl)pyridin-2-yl)-1H-1,2,4-triazol-3-yl)phenyl)-3-(3-(2-isopropyl-5-methoxyphenyl)-4-oxothiazolidin-2-ylidene)urea (J283)

The title compound was prepared from 2-imino-3-(2-isopropyl-5-methoxyphenyl)thiazolidin-4-one (C98) and was isolated as an off-white solid (70 mg, 72%).

### (Z)-1-(3-(2-Isopropyl-5-methylphenyl)-4-oxothiazolidin-2-ylidene)-3-(2-methyl-4-(1-(5-(trifluoromethyl)pyridin-2-yl)-1H-1,2,4-triazol-3-yl)phenyl)urea (J284)

The title compound was prepared from 2-imino-3-(2-isopropyl-5-methylphenyl)thiazolidin-4-one (prepared as in U.S. Patent Application Publication 20140274688 A1) and was isolated as an off-white solid (84 mg, 86%).

### (Z)-1-(3-(5-(Dimethylamino)-2-isopropylphenyl)-4-oxothiazolidin-2-ylidene)-3-(2-methyl-4-(1-(5-(trifluoromethyl)pyridin-2-yl)-1H-1,2,4-triazol-3-yl)phenyl)urea (J285)

The title compound was prepared from 3-(5-(dimethylamino)-2-isopropylphenyl)-2-iminothiazolidin-4-one (C126) and was isolated as a white solid (70 mg, 70%).

### (Z)-1-(3-(2-(1-Methoxyethyl)-5-methylphenyl)-4-oxothiazolidin-2-ylidene)-3-(2-methyl-4-(1-(5-(trifluoromethyl)pyridin-2-yl)-1H-1,2,4-triazol-3-yl)phenyl)urea (J286)

The title compound was prepared from 2-imino-3-(2-(1-methoxyethyl)-5-methylphenyl)-thiazolidin-4-one (prepared as in PCT International Application Publication WO 2017040194 A1) and was isolated as a white solid (66 mg, 68%).

### (Z)-1-(3-(2-Isopropyl-5-methoxyphenyl)-4-oxothiazolidin-2-ylidene)-3-(2-methyl-4-(1-(5-(trifluoromethyl)pyridin-2-yl)-1H-1,2,4-triazol-3-yl)phenyl)urea (J292)

The title compound was prepared from 2-imino-3-(2-isopropyl-5-methoxyphenyl)thiazolidin-4-one (C98) and was isolated as an off-white solid (66 mg, 68%).

### (Z)-1-(3-(5-Methyl-2-propylphenyl)-4-oxothiazolidin-2-ylidene)-3-(2-methyl-4-(1-(5-(trifluoromethyl)pyridin-2-yl)-1H-1,2,4-triazol-3-yl)phenyl)urea (J293)

The title compound was prepared from 2-imino-3-(5-methyl-2-propylphenyl)thiazolidin-4-one (prepared as in PCT International Application Publication WO 2016033025 A1) and was isolated as a white solid (47 mg, 50%).

### (Z)-1-(3-(5-(Dimethylamino)-2-propylphenyl)-4-oxothiazolidin-2-ylidene)-3-(2-methyl-4-(1-(5-(trifluoromethyl)pyridin-2-yl)-1H-1,2,4-triazol-3-yl)phenyl)urea (J294)

The title compound was prepared from 3-(5-(dimethylamino)-2-propylphenyl)-2-iminothiazolidin-4-one (C128) and was isolated as an off-white solid (67 mg, 67%).

### (Z)-1-(2-Chloro-4-(1-(5-(trifluoromethyl)pyridin-2-yl)-1H-1,2,4-triazol-3-yl)phenyl)-3-(3-(2-isopropyl-5-methylphenyl)-4-oxothiazolidin-2-ylidene)urea (J295)

The title compound was prepared from 2-imino-3-(2-isopropyl-5-methylphenyl)thiazolidin-4-one (prepared as in U.S. Patent Application Publication 20140274688 A1) and was isolated as an off-white solid (22 mg, 23%).

### (Z)-1-(2-Chloro-4-(1-(5-(trifluoromethyl)pyridin-2-yl)-1H-1,2,4-triazol-3-yl)phenyl)-3-(3-(5-(dimethylamino)-2-isopropylphenyl)-4-oxothiazolidin-2-ylidene)urea (J296)

The title compound was prepared from 3-(5-(dimethylamino)-2-isopropylphenyl)-2-iminothiazolidin-4-one (C126) and was isolated as an off-white solid (32 mg, 32%).

### (Z)-1-(3-(5-Methyl-2-((2,2,2-trifluoroethoxy)methyl)phenyl)-4-oxothiazolidin-2-ylidene)-3-(2-methyl-4-(1-(5-(trifluoromethyl)pyridin-2-yl)-1H-1,2,4-triazol-3-yl)phenyl)urea (J297)

The title compound was prepared from 2-imino-3-(5-methyl-2-((2,2,2-trifluoroethoxy)methyl)phenyl)thiazolidin-4-one (C117) and was isolated as an off-white solid (37 mg, 35%).

### (Z)-1-(2-Fluoro-4-(1-(5-(trifluoromethyl)pyridin-2-yl)-1H-1,2,4-triazol-3-yl)phenyl)-3-(3-(2-(methoxymethyl)-5-methylphenyl)-4-oxothiazolidin-2-ylidene)urea (J298)

The title compound was prepared from 2-imino-3-(2-(methoxymethyl)-5-methylphenyl)thiazolidin-4-one (prepared as in PCT International Application Publication WO 2017040742 A1) and was isolated as a yellow solid (29 mg, 31%).

### (Z)-1-(3-(2-(Ethoxymethyl)-5-methylphenyl)-4-oxothiazolidin-2-ylidene)-3-(2-fluoro-4-(1-(5-(trifluoromethyl)pyridin-2-yl)-1H-1,2,4-triazol-3-yl)phenyl)urea (J299)

The title compound was prepared from 3-(2-(ethoxymethyl)-5-methylphenyl)-2-iminothiazolidin-4-one (C120) and was isolated as a white solid (58 mg, 60%).

### (Z)-1-(2-Fluoro-4-(1-(5-(trifluoromethyl)pyridin-2-yl)-1H-1,2,4-triazol-3-yl)phenyl)-3-(3-(2-(1-methoxyethyl)-4-methylphenyl)-4-oxothiazolidin-2-ylidene)urea (J300)

The title compound was prepared from 2-imino-3-(2-(1-methoxyethyl)-4-methylphenyl)thiazolidin-4-one (C144) and was isolated as a white solid (56 mg, 58%).

### (Z)-1-(2-Chloro-4-(1-(5-(trifluoromethyl)pyridin-2-yl)-1H-1,2,4-triazol-3-yl)phenyl)-3-(3-(2-(1-methoxyethyl)-5-methylphenyl)-4-oxothiazolidin-2-ylidene)urea (J306)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (42 mg, 44%).

### (Z)-1-(2-Fluoro-4-(1-(5-(trifluoromethyl)pyridin-2-yl)-1H-1,2,4-triazol-3-yl)phenyl)-3-(3-(5-methyl-2-(3,3,3-trifluoropropoxy)phenyl)-4-oxothiazolidin-2-ylidene)urea (J307)

The title compound was prepared from 2-imino-3-(5-methyl-2-(3,3,3-trifluoropropoxy)phenyl)thiazolidin-4-one (C99) and was isolated as an off-white solid (61 mg, 58%).

### (Z)-1-(2-Fluoro-4-(1-(5-(trifluoromethyl)pyridin-2-yl)-1H-pyrazol-3-yl)phenyl)-3-(3-(5-methyl-2-((2,2,2-trifluoroethoxy)methyl)phenyl)-4-oxothiazolidin-2-ylidene)urea (J308)

The title compound was prepared from 2-imino-3-(5-methyl-2-((2,2,2-trifluoroethoxy)methyl)phenyl)thiazolidin-4-one (C117) and was isolated as a white solid (66 mg, 63%).

### (Z)-1-(2-Fluoro-4-(1-(5-(trifluoromethyl)pyridin-2-yl)-1H-pyrazol-3-yl)phenyl)-3-(3-(2-(1-methoxyethyl)-5-methylphenyl)-4-oxothiazolidin-2-ylidene)urea (J309)

The title compound was prepared from 2-imino-3-(2-(1-methoxyethyl)-5-methylphenyl)-thiazolidin-4-one (prepared as in PCT International Application Publication WO 2017040194 A1) and was isolated as a white solid (42 mg, 43%).

### (Z)-1-(3-(5-Methyl-2-((2,2,2-trifluoroethoxy)methyl)phenyl)-4-oxothiazolidin-2-ylidene)-3-(2-methyl-4-(1-(5-(trifluoromethyl)pyridin-2-yl)-1H-pyrazol-3-yl)phenyl)urea (J310)

The title compound was prepared from 2-imino-3-(5-methyl-2-((2,2,2-trifluoroethoxy)methyl)phenyl)thiazolidin-4-one (C117) and was isolated as a white solid (75 mg, 71%).

### (Z)-1-(3-(2-(1-Methoxyethyl)-5-methylphenyl)-4-oxothiazolidin-2-ylidene)-3-(2-methyl-4-(1-(5-(trifluoromethyl)pyridin-2-yl)-1H-pyrazol-3-yl)phenyl)urea (J311)

The title compound was prepared from 2-imino-3-(2-(1-methoxyethyl)-5-methylphenyl)-thiazolidin-4-one (prepared as in PCT International Application Publication WO 2017040194 A1) and was isolated as a white solid (78 mg, 81%).

### Example 32: Preparation of (Z)-1-(3-(5-(diethylamino)-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J69)

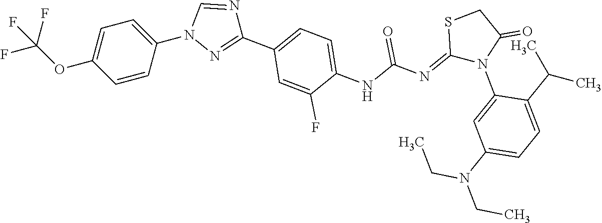 [SMILES: CCN(CC)c1ccc(C(C)C)c(N2C(=O)CS/C2=N\C(=O)Nc2ccc(-c3ncn(-c4ccc(OC(F)(F)F)cc4)n3)cc2F)c1]

2-Fluoro-4-(1-(4-(trifluoromethoxy)phenyl)-1H-1,2,4-triazol-3-yl)aniline (C55; 0.075 g, 0.222 mmol) was suspended in DCM (1.00 mL), and N,N-diisopropylethylamine (0.116 mL, 0.665 mmol) was added. Once the solution was homogeneous, triphosgene (0.026 g, 0.089 mmol) was slowly added in portions. After consumption of the starting material, 3-(5-(diethylamino)-2-isopropylphenyl)-2-iminothiazolidin-4-one (C130; 0.068 g, 0.222 mmol) was added. The mixture was stirred for 1 h and was concentrated onto diatomaceous earth. Purification via silica gel chromatography eluting with 0-40% acetone-hexanes provided the title compound as an off-white foam (77 mg, 52%).

The following compound was prepared in accordance to the procedure in Example 32.

### (Z)-1-(2,6-Difluoro-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J78)

The title compound was prepared from 2-imino-3-(2-isopropyl-5-methylphenyl)thiazolidin-4-one (prepared as in U.S. Patent Application Publication 20140274688 A1) and was isolated a yellow powder (44 mg, 50%).

### (Z)-1-(2,6-Difluoro-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J79)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as a yellow powder (58 mg, 60%).

### (Z)-1-(2,6-Difluoro-4-(1-(4-(trifluoromethoxy)phenyl)-1H-1,2,4-triazol-3-yl)phenyl)-3-(3-(5-methyl-2-(4,4,4-trifluorobutoxy)phenyl)-4-oxothiazolidin-2-ylidene)urea (J80)

The title compound was prepared from 2-imino-3-(5-methyl-2-(4,4,4-trifluorobutoxy)phenyl)thiazolidin-4-one (C107) and was isolated as a yellow powder (51 mg, 51%).

### (Z)-1-(2,6-Difluoro-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J81)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow powder (49 mg, 54%).

### (Z)-1-(2,6-Difluoro-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J82)

The title compound was prepared from 3-(5-(dimethylamino)-2-isopropylphenyl)-2-iminothiazolidin-4-one (C126) and was isolated as a yellow powder (53 mg, 57%).

### (Z)-1-(2,6-Dimethyl-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J95)

The title compound was prepared from 2-imino-3-(2-isopropyl-5-methylphenyl)thiazolidin-4-one (prepared as in U.S. Patent Application Publication 20140274688 A1) and was isolated as a white powder (51 mg, 71%).

### (Z)-1-(2,6-Dimethyl-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J96)

The title compound was prepared from 3-(5-(dimethylamino)-2-isopropylphenyl)-2-iminothiazolidin-4-one (C126) and was isolated as an off-white powder (57 mg, 76%).

### (Z)-1-(2,6-Dimethyl-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J97)

The title compound was prepared from 2-imino-3-(2-(1-methoxyethyl)-5-methylphenyl)-thiazolidin-4-one (prepared as in PCT International Application Publication WO 2017040194 A1) and was isolated as a white powder (44 mg, 80%).

### (Z)-1-(2,6-Dimethyl-4-(1-(4-(trifluoromethoxy)phenyl)-1H-1,2,4-triazol-3-yl)phenyl)-3-(3-(2-isopropyl-5-methoxyphenyl)-4-oxothiazolidin-2-ylidene)urea (J98)

The title compound was prepared from 2-imino-3-(2-isopropyl-5-methoxyphenyl)thiazolidin-4-one (C98) and was isolated as a white powder (47 mg, 85%).

### (Z)-1-(3-(5-(Ethylamino)-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J106)

The title compound was prepared from 3-(5-(ethylamino)-2-isopropylphenyl)-2-iminothiazolidin-4-one hydrochloride (C151) and was isolated as a white solid (46 mg, 49%).

### (Z)-1-(2-Fluoro-4-(1-(4-(trifluoromethoxy)phenyl)-1H-1,2,4-triazol-3-yl)phenyl)-3-(3-(2-isopropyl-5-(propylamino)phenyl)-4-oxothiazolidin-2-ylidene)urea (J107)

The title compound was prepared from 2-imino-3-(2-isopropyl-5-(propylamino)phenyl)thiazolidin-4-one hydrochloride (C152) and was isolated as a white solid (62 mg, 64%).

### (Z)-1-(3-(5-(Dipropylamino)-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J108)

The title compound was prepared from 3-(5-(dipropylamino)-2-isopropylphenyl)-2-iminothiazolidin-4-one (C131) and was isolated as a yellow powder (57 mg, 55%).

### (Z)-1-(3-(2-Isopropyl-5-methylphenyl)-4-oxothiazolidin-2-ylidene)-3-(4-(1-(4-(trifluoromethoxy)phenyl)-1H-1,2,4-triazol-3-yl)-2-(trifluoromethyl)phenyl)urea (J114)

The title compound was prepared from 2-imino-3-(2-isopropyl-5-methylphenyl)thiazolidin-4-one (prepared as in U.S. Patent Application Publication 20140274688 A1) and was isolated as a yellow powder (39 mg, 33%).

### (Z)-1-(3-(5-Methyl-2-(2,2,2-trifluoroethoxy)phenyl)-4-oxothiazolidin-2-ylidene)-3-(4-(1-(4-(trifluoromethoxy)phenyl)-1H-1,2,4-triazol-3-yl)-2-(trifluoromethyl)phenyl)urea (J115)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as a yellow powder (61 mg, 47%).

### (Z)-1-(3-(5-(Dimethylamino)-2-isopropylphenyl)-4-oxothiazolidin-2-ylidene)-3-(4-(1-(4-(trifluoromethoxy)phenyl)-1H-1,2,4-triazol-3-yl)-2-(trifluoromethyl)phenyl)urea (J116)

The title compound was prepared from 3-(5-(dimethylamino)-2-isopropylphenyl)-2-iminothiazolidin-4-one (C126) and was isolated as a yellow powder (43 mg, 35%).

### (Z)-1-(3-(2-(1-Methoxyethyl)-5-methylphenyl)-4-oxothiazolidin-2-ylidene)-3-(4-(1-(4-(trifluoromethoxy)phenyl)-1H-1,2,4-triazol-3-yl)-2-(trifluoromethyl)phenyl)urea (J117)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow powder (40 mg, 33%).

### (Z)-1-(3-(5-(Dimethylamino)-2-(3,3,3-trifluoropropoxy)phenyl)-4-oxothiazolidin-2-ylidene)-3-(4-(1-(4-(trifluoromethoxy)phenyl)-1H-1,2,4-triazol-3-yl)-2-(trifluoromethyl)phenyl)urea (J118)

The title compound was prepared from 3-(5-(dimethylamino)-2-(3,3,3-trifluoropropoxy)phenyl)-2-iminothiazolidin-4-one (C101) and was isolated as a yellow powder (85 mg, 62%).

### (Z)-1-(3-(2-Isopropyl-5-(methylamino)phenyl)-4-oxothiazolidin-2-ylidene)-3-(2-methyl-4-(1-(4-(trifluoromethoxy)phenyl)-1H-1,2,4-triazol-3-yl)phenyl)urea (J126)

The title compound was prepared from 2-imino-3-(2-isopropyl-5-(methylamino)phenyl)thiazolidin-4-one hydrochloride (C150) and was isolated as an off-white solid (80 mg, 61%).

### (Z)-1-(3-(5-(Ethylamino)-2-isopropylphenyl)-4-oxothiazolidin-2-ylidene)-3-(2-methyl-4-(1-(4-(trifluoromethoxy)phenyl)-1H-1,2,4-triazol-3-yl)phenyl)urea (J127)

The title compound was prepared from 3-(5-(ethylamino)-2-isopropylphenyl)-2-iminothiazolidin-4-one hydrochloride (C151) and was isolated as an off-white solid (68 mg, 51%).

### (Z)-1-(3-(2-Isopropyl-5-(propylamino)phenyl)-4-oxothiazolidin-2-ylidene)-3-(2-methyl-4-(1-(4-(trifluoromethoxy)phenyl)-1H-1,2,4-triazol-3-yl)phenyl)urea (J128)

The title compound was prepared from 2-imino-3-(2-isopropyl-5-(propylamino)phenyl)thiazolidin-4-one hydrochloride (C152) and was isolated as an off-white solid (95 mg, 70%).

### (Z)-1-(3-(5-(Diethylamino)-2-isopropylphenyl)-4-oxothiazolidin-2-ylidene)-3-(2-methyl-4-(1-(4-(trifluoromethoxy)phenyl)-1H-1,2,4-triazol-3-yl)phenyl)urea (J129)

The title compound was prepared from 3-(5-(diethylamino)-2-isopropylphenyl)-2-iminothiazolidin-4-one (C130) and was isolated as an off-white solid (109 mg, 78%).

### (Z)-1-(3-(5-(Dipropylamino)-2-isopropylphenyl)-4-oxothiazolidin-2-ylidene)-3-(2-methyl-4-(1-(4-(trifluoromethoxy)phenyl)-1H-1,2,4-triazol-3-yl)phenyl)urea (J130)

The title compound was prepared from 3-(5-(dipropylamino)-2-isopropylphenyl)-2-iminothiazolidin-4-one (C131) and was isolated as an off-white solid (111 mg, 76%).

### (Z)-1-(2-Ethyl-4-(1-(4-(trifluoromethoxy)phenyl)-1H-1,2,4-triazol-3-yl)phenyl)-3-(3-(2-isopropyl-5-(methylamino)phenyl)-4-oxothiazolidin-2-ylidene)urea (J157)

The title compound was prepared from 2-imino-3-(2-isopropyl-5-(methylamino)phenyl)thiazolidin-4-one hydrochloride (C150) and was isolated as an off-white solid (61 mg, 48%).

### (Z)-1-(2-Ethyl-4-(1-(4-(trifluoromethoxy)phenyl)-1H-1,2,4-triazol-3-yl)phenyl)-3-(3-(5-(ethylamino)-2-isopropylphenyl)-4-oxothiazolidin-2-ylidene)urea (J158)

The title compound was prepared from 3-(5-(ethylamino)-2-isopropylphenyl)-2-iminothiazolidin-4-one hydrochloride (C151) and was isolated as an off-white solid (63 mg, 48%).

### (Z)-1-(2-Ethyl-4-(1-(4-(trifluoromethoxy)phenyl)-1H-1,2,4-triazol-3-yl)phenyl)-3-(3-(2-isopropyl-5-(propylamino)phenyl)-4-oxothiazolidin-2-ylidene)urea (J159)

The title compound was prepared from 2-imino-3-(2-isopropyl-5-(propylamino)phenyl)thiazolidin-4-one hydrochloride (C152) and was isolated as an off-white solid (79 mg, 59%).

### (Z)-1-(3-(5-(Diethylamino)-2-isopropylphenyl)-4-oxothiazolidin-2-ylidene)-3-(2-ethyl-4-(1-(4-(trifluoromethoxy)phenyl)-1H-1,2,4-triazol-3-yl)phenyl)urea (J160)

The title compound was prepared from 3-(5-(diethylamino)-2-isopropylphenyl)-2-iminothiazolidin-4-one (C130) and was isolated as an off-white solid (75 mg, 55%).

### (Z)-1-(3-(5-(Dipropylamino)-2-isopropylphenyl)-4-oxothiazolidin-2-ylidene)-3-(2-ethyl-4-(1-(4-(trifluoromethoxy)phenyl)-1H-1,2,4-triazol-3-yl)phenyl)urea (J161)

The title compound was prepared from 3-(5-(dipropylamino)-2-isopropylphenyl)-2-iminothiazolidin-4-one (C131) and was isolated as an off-white solid (75 mg, 53%).

### (Z)-1-(2-Fluoro-4-(1-(4-(trifluoromethoxy)phenyl)-1H-1,2,4-triazol-3-yl)phenyl)-3-(3-(5-methyl-2-nitrophenyl)-4-oxothiazolidin-2-ylidene)urea (J163)

The title compound was prepared from 2-imino-3-(5-methyl-2-nitrophenyl)thiazolidin-4-one (C136) and was isolated as a yellow solid (400 mg, 73%).

### (Z)-1-(3-(5-Methyl-2-((2,2,2-trifluoroethoxy)methyl)phenyl)-4-oxothiazolidin-2-ylidene)-3-(4-(1-(4-(trifluoromethoxy)phenyl)-1H-1,2,4-triazol-3-yl)-2-(trifluoromethyl)phenyl)urea (J165)

The title compound was prepared from 2-imino-3-(5-methyl-2-((2,2,2-trifluoroethoxy)methyl)phenyl)thiazolidin-4-one (C117) and was isolated as a yellow solid (50 mg, 27%).

### (Z)-1-(3-(2-Isopropyl-5-methylphenyl)-4-oxothiazolidin-2-ylidene)-3-(2-(methoxymethoxy)-4-(1-(4-(trifluoromethoxy)phenyl)-1H-1,2,4-triazol-3-yl)phenyl)urea (J170)

The title compound was prepared from 2-imino-3-(2-isopropyl-5-methylphenyl)thiazolidin-4-one (prepared as in U.S. Patent Application Publication 20140274688 A1) and was isolated as an off-white powder (192 mg, 74%).

### (Z)-1-(2-(Methoxymethoxy)-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J171)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white solid (178 mg, 64%).

### (Z)-1-(3-(5-(Dimethylamino)-2-isopropylphenyl)-4-oxothiazolidin-2-ylidene)-3-(2-(methoxymethoxy)-4-(1-(4-(trifluoromethoxy)phenyl)-1H-1,2,4-triazol-3-yl)phenyl)urea (J172)

The title compound was prepared from 3-(5-(dimethylamino)-2-isopropylphenyl)-2-iminothiazolidin-4-one (C126) and was isolated as an off-white solid (240 mg, 89%).

### (Z)-1-(2-(Methoxymethoxy)-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J173)

The title compound was prepared from 2-imino-3-(5-methyl-2-((2,2,2-trifluoroethoxy)methyl)phenyl)thiazolidin-4-one (C117) and was isolated as an off-white solid (182 mg, 64%).

### (Z)-1-(3-(2-(1-Ethoxyethyl)-5-methylphenyl)-4-oxothiazolidin-2-ylidene)-3-(2-(methoxymethoxy)-4-(1-(4-(trifluoromethoxy)phenyl)-1H-1,2,4-triazol-3-yl)phenyl)urea (J174)

The title compound was prepared from 3-(2-(1-ethoxyethyl)-5-methylphenyl)-2-iminothiazolidin-4-one (C118) and was isolated as an off-white solid (233 mg, 86%).

### (Z)-N-(3-(2-(((2-Fluoro-4-(1-(4-(trifluoromethoxy)phenyl)-1H-1,2,4-triazol-3-yl)phenyl)carbamoyl)imino)-4-oxothiazolidin-3-yl)-4-isopropylphenyl)-N-methylacetamide (J188)

The title compound was prepared from N-(3-(2-imino-4-oxothiazolidin-3-yl)-4-isopropylphenyl)-N-methylacetamide (C138) and was isolated as an off-white semi-solid (118 mg, 75%).

### (Z)-N-Ethyl-N-(3-(2-(((2-fluoro-4-(1-(4-(trifluoromethoxy)phenyl)-1H-1,2,4-triazol-3-yl)phenyl)carbamoyl)imino)-4-oxothiazolidin-3-yl)-4-isopropylphenyl)acetamide (J189)

The title compound was prepared from N-ethyl-N-(3-(2-imino-4-oxothiazolidin-3-yl)-4-isopropylphenyl)acetamide (C139) and was isolated as an off-white solid (135 mg, 83%).

### (Z)-N-(4-Isopropyl-3-(2-(((2-methyl-4-(1-(4-(trifluoromethoxy)phenyl)-1H-1,2,4-triazol-3-yl)phenyl)carbamoyl)imino)-4-oxothiazolidin-3-yl)phenyl)-N-methylacetamide (J199)

The title compound was prepared from N-(3-(2-imino-4-oxothiazolidin-3-yl)-4-isopropylphenyl)-N-methylacetamide (C138) and was isolated as a white powder (35 mg, 35%).

### (Z)-N-Ethyl-N-(4-isopropyl-3-(2-(((2-methyl-4-(1-(4-(trifluoromethoxy)phenyl)-1H-1,2,4-triazol-3-yl)phenyl)carbamoyl)imino)-4-oxothiazolidin-3-yl)phenyl)acetamide (J200)

The title compound was prepared from N-ethyl-N-(3-(2-imino-4-oxothiazolidin-3-yl)-4-isopropylphenyl)acetamide (C139) and was isolated as a white powder (44 mg, 43%).

### (Z)-1-(2-(Difluoromethyl)-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J211)

The title compound was prepared from 2-imino-3-(2-isopropyl-5-methylphenyl)thiazolidin-4-one (prepared as in U.S. Patent Application Publication 20140274688 A1) and was isolated as an off-white foam (58 mg, 37%).

### (Z)-1-(2-(Difluoromethyl)-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J212)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as an off-white foam (49 mg, 29%).

### (Z)-1-(2-(Difluoromethyl)-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J213)

The title compound was prepared from 3-(5-(dimethylamino)-2-isopropylphenyl)-2-iminothiazolidin-4-one (C126) and was isolated as an off-white foam (52 mg, 32%).

### (Z)-1-(2-(Difluoromethyl)-4-(1-(4-(trifluoromethoxy)phenyl)-1H-1,2,4-triazol-3-yl)phenyl)-3-(3-(2-(ethoxymethyl)-5-methylphenyl)-4-oxothiazolidin-2-ylidene)urea (J214)

The title compound was prepared from 3-(2-(ethoxymethyl)-5-methylphenyl)-2-iminothiazolidin-4-one (C120) and was isolated as an off-white foam (50 mg, 31%).

### (Z)-1-(2-(Difluoromethyl)-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J215)

The title compound was prepared from 2-imino-3-(5-methyl-2-((2,2,2-trifluoroethoxy)methyl)phenyl)thiazolidin-4-one (C117) and was isolated as an off-white foam (54 mg, 31%).

### (Z)-1-(2-Bromo-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J217)

The title compound was prepared from 2-imino-3-(2-isopropyl-5-methylphenyl)thiazolidin-4-one (prepared as in U.S. Patent Application Publication 20140274688 A1) and was isolated as a yellow foamy powder (97 mg, 77%).

### (Z)-1-(2-Bromo-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J218)

The title compound was prepared from 2-imino-3-(5-methyl-2-(2,2,2-trifluoroethoxy)phenyl)thiazolidin-4-one (prepared as in PCT International Application Publication WO 2017040742 A1) and was isolated as a yellow foam (70 mg, 51%).

### (Z)-1-(2-Bromo-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J219)

The title compound was prepared from 3-(5-(dimethylamino)-2-isopropylphenyl)-2-iminothiazolidin-4-one (C126) and was isolated as a yellow foam (94 mg, 71%).

### (Z)-1-(2-Bromo-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J220)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow foam (56 mg, 43%).

### (Z)-1-(2-Bromo-4-(1-(4-(trifluoromethoxy)phenyl)-1H-1,2,4-triazol-3-yl)phenyl)-3-(3-(2-(ethoxymethyl)-5-methylphenyl)-4-oxothiazolidin-2-ylidene)urea (J221)

The title compound was prepared from 3-(2-(ethoxymethyl)-5-methylphenyl)-2-iminothiazolidin-4-one (C120) and was isolated as a yellow foam (93 mg, 72%).

### (Z)-1-(2-Bromo-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J222)

The title compound was prepared from 2-imino-3-(5-methyl-2-((2,2,2-trifluoroethoxy)methyl)phenyl)thiazolidin-4-one (C117) and was isolated as a yellow foam (107 mg, 77%).

### (Z)-1-(4-(1-(4-Cyanophenyl)-1H-1,2,4-triazol-3-yl)-2-fluorophenyl)-3-(3-(2-isopropyl-5-methylphenyl)-4-oxothiazolidin-2-ylidene)urea (J287)

The title compound was prepared from 2-imino-3-(2-isopropyl-5-methylphenyl)thiazolidin-4-one (prepared as in U.S. Patent Application Publication 20140274688 A1) and was isolated as a yellow powder (64 mg, 43%).

### (Z)-1-(4-(1-(4-Cyanophenyl)-1H-1,2,4-triazol-3-yl)-2-fluorophenyl)-3-(3-(5-methyl-2-(3,3,3-trifluoropropoxy)phenyl)-4-oxothiazolidin-2-ylidene)urea (J288)

The title compound was prepared from 2-imino-3-(5-methyl-2-(3,3,3-trifluoropropoxy)phenyl)thiazolidin-4-one (C99) and was isolated as a yellow powder (114 mg, 68%).

### (Z)-1-(4-(1-(4-Cyanophenyl)-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J289)

The title compound was prepared from 2-imino-3-(5-methyl-2-((2,2,2-trifluoroethoxy)methyl)phenyl)thiazolidin-4-one (C117) and was isolated as a yellow powder (36 mg, 22%).

### (Z)-1-(4-(1-(4-Cyanophenyl)-1H-1,2,4-triazol-3-yl)-2-fluorophenyl)-3-(3-(5-(dimethylamino)-2-isopropylphenyl)-4-oxothiazolidin-2-ylidene)urea (J290)

The title compound was prepared from 3-(5-(dimethylamino)-2-isopropylphenyl)-2-iminothiazolidin-4-one (C126) and was isolated as a yellow powder (116 mg, 74%).

### (Z)-1-(4-(1-(4-Cyanophenyl)-1H-1,2,4-triazol-3-yl)-2-fluorophenyl)-3-(3-(2-(1-methoxyethyl)-5-methylphenyl)-4-oxothiazolidin-2-ylidene)urea (J291)

The title compound was prepared from 2-imino-3-(2-(1-methoxyethyl)-5-methylphenyl)-thiazolidin-4-one (prepared as in PCT International Application Publication WO 2017040194 A1) and was isolated as a yellow powder (52 mg, 34%).

### Example 33: Preparation of (Z)-1-(2-hydroxy-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J175)

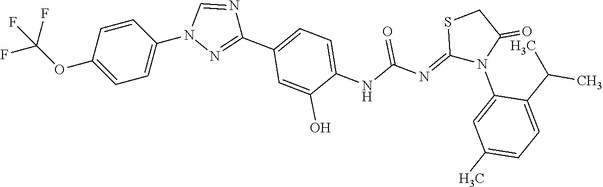 [SMILES: Cc1ccc(C(C)C)c(N2C(=O)CS/C2=N\C(=O)Nc2ccc(-c3ncn(-c4ccc(OC(F)(F)F)cc4)n3)cc2O)c1]

(Z)-1-(3-(2-Isopropyl-5-methylphenyl)-4-oxothiazolidin-2-ylidene)-3-(2-(methoxymethoxy)-4-(1-(4-(trifluoromethoxy)phenyl)-1H-1,2,4-triazol-3-yl)phenyl)urea (J170; 0.112 g, 0.171 mmol) was dissolved in THF (0.855 mL), and 10% aq HCl (0.520 mL, 1.711 mmol) was added. The mixture was heated to 65° C. overnight, and solid began to precipitate. The mixture was concentrated and directly loaded onto diatomaceous earth. Purification by silica gel chromatography eluting with 0-40% acetone-hexanes afforded the title compound as a yellow powder (0.028 g, 27%).

The following compounds were prepared in accordance to the procedure in Example 33.

### (Z)-1-(2-Hydroxy-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J176)

The title compound was prepared from (Z)-1-(2-(methoxymethoxy)-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)ure (J171) and was isolated as an off-white powder (29 mg, 29%).

### (Z)-1-(3-(5-(Dimethylamino)-2-isopropylphenyl)-4-oxothiazolidin-2-ylidene)-3-(2-hydroxy-4-(1-(4-(trifluoromethoxy)phenyl)-1H-1,2,4-triazol-3-yl)phenyl)urea (J177)

The title compound was prepared from (Z)-1-(3-(5-(dimethylamino)-2-isopropylphenyl)-4-oxothiazolidin-2-ylidene)-3-(2-(methoxymethoxy)-4-(1-(4-(trifluoromethoxy)phenyl)-1H-1,2,4-triazol-3-yl)phenyl)urea (J172) and was isolated as an off-white powder (7 mg, 4.4%).

### (Z)-1-(2-Hydroxy-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J178)

The title compound was prepared from (Z)-1-(2-(methoxymethoxy)-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J173) and was isolated as an off-white powder (22 mg, 21%).

### (Z)-1-(3-(2-(1-Ethoxyethyl)-5-methylphenyl)-4-oxothiazolidin-2-ylidene)-3-(2-hydroxy-4-(1-(4-(trifluoromethoxy)phenyl)-1H-1,2,4-triazol-3-yl)phenyl)urea (J179)

The title compound was prepared from (Z)-1-(3-(2-(1-ethoxyethyl)-5-methylphenyl)-4-oxothiazolidin-2-ylidene)-3-(2-(methoxymethoxy)-4-(1-(4-(trifluoromethoxy)phenyl)-1H-1,2,4-triazol-3-yl)phenyl)urea (J174) and was isolated as an off-white powder (43 mg, 28%).

### Example 34: Preparation of (Z)-1-(5-chloro-3-(2-isopropyl-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J32)

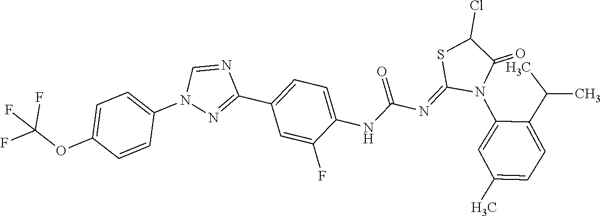 [SMILES: Cc1ccc(C(C)C)c(N2C(=O)C(Cl)S/C2=N\C(=O)Nc2ccc(-c3ncn(-c4ccc(OC(F)(F)F)cc4)n3)cc2F)c1]

To a vial containing (Z)-1-(2-fluoro-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J19; 102 mg, 0.167 mmol) and 1-chloropyrrolidine-2,5-dione (26.7 mg, 0.200 mmol) was added DMF (1.70 mL), and the reaction mixture was stirred at 40° C. Upon completion, the reaction mixture was washed with a mixture of methyl tert-butyl ether (MTBE; ˜15 mL) and water (˜10 mL). The layers were separated and the aqueous layer was extracted with DCM (10 mL). The combined organic layers were dried over Na₂SO₄, filtered, and concentrated, and then were loaded onto a silica cartridge. Purification by flash chromatography (silica gel, 0-100% EtOAc-(1:1 hexanes-DCM)) yielded the title compound as an off-white foam (22 mg, 19%).

The following compounds were prepared in accordance to the procedure in Example 34.

### (Z)-1-(5-Chloro-3-(5-methyl-2-(2,2,2-trifluoroethoxy)phenyl)-4-oxothiazolidin-2-ylidene)-3-(2-fluoro-4-(1-(4-(trifluoromethoxy)phenyl)-1H-1,2,4-triazol-3-yl)phenyl)urea (J33)

The title compound was prepared from (Z)-1-(2-fluoro-4-(1-(4-(trifluoromethoxy)phenyl)-1H-1,2,4-triazol-3-yl)phenyl)-3-(3-(5-methyl-2-(2,2,2-trifluoroethoxy)phenyl)-4-oxothiazolidin-2-ylidene)urea (J31) and was isolated as an off-white foam (20 mg, 14%).

### (Z)-1-(5-Chloro-3-(5-methyl-2-(trifluoromethyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J34)

The title compound was prepared from (Z)-1-(2-fluoro-4-(1-(4-(trifluoromethoxy)phenyl)-1H-1,2,4-triazol-3-yl)phenyl)-3-(3-(5-methyl-2-(trifluoromethyl)phenyl)-4-oxothiazolidin-2-ylidene)urea (J30) and was isolated as an off-white foam (24 mg, 19%).

### (Z)-1-(5-Chloro-3-(5-methyl-2-(3,3,3-trifluoropropoxy)phenyl)-4-oxothiazolidin-2-ylidene)-3-(2-fluoro-4-(1-(4-(trifluoromethoxy)phenyl)-1H-1,2,4-triazol-3-yl)phenyl)urea (J36)

The title compound was prepared from (Z)-1-(2-fluoro-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J35) and was isolated as a yellow foam (29 mg, 15%).

### Example 35: Preparation of (Z)-1-(2-cyano-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J227)

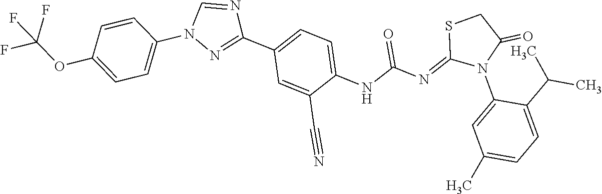 [SMILES: Cc1ccc(C(C)C)c(N2C(=O)CS/C2=N\C(=O)Nc2ccc(-c3ncn(-c4ccc(OC(F)(F)F)cc4)n3)cc2C#N)c1]

THF (1 mL) was used to solublize 2-amino-5-(1-(4-(trifluoromethoxy)phenyl)-1H-1,2,4-triazol-3-yl)benzonitrile (C86; 0.045 g, 0.130 mmol) and 4-nitrophenyl carbonochloridate (0.026 g, 0.130 mmol). After 30 min, the mixture was a white, opaque slurry. 2-Imino-3-(2-isopropyl-5-methylphenyl)thiazolidin-4-one (prepared as in U.S. Patent Application Publication 20140274688 A1; 0.036 g, 0.145 mmol) and N,N-diisopropylethylamine (0.68 mL, 0.391 mmol) were added. The mixture became homogenous and was allowed to stir for 1 h. The mixture was concentrated onto diatomaceous earth. Purification by silica gel chromatography eluting with 0-40% acetone-hexanes provided the title compound as a pink solid (39 mg, 44%).

The following compounds were prepared in accordance to the procedure in Example 35.

### (Z)-1-(2-Cyano-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J228)

The title compound was prepared from 2-imino-3-(2-(1-methoxyethyl)-5-methylphenyl)-thiazolidin-4-one (prepared as in PCT International Application Publication WO 2017040194 A1) and was isolated as an orange foam (27 mg, 33%).

### (Z)-1-(2-Cyano-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J242)

The title compound was prepared from 3-(5-(dimethylamino)-2-isopropylphenyl)-2-iminothiazolidin-4-one (C126) and was isolated as an off-white solid (48 mg, 34%).

### (Z)-1-(2-Cyano-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J243)

The title compound was prepared from 2-imino-3-(5-methyl-2-(3,3,3-trifluoropropoxy)phenyl)thiazolidin-4-one (C99) and was isolated as an off-white powder (72 mg, 48%).

### (Z)-1-(2-Cyano-4-(1-(4-(trifluoromethoxy)phenyl)-1H-1,2,4-triazol-3-yl)phenyl)-3-(3-(5-methyl-2-(trifluoromethyl)phenyl)-4-oxothiazolidin-2-ylidene)urea (J244)

The title compound was prepared from 2-imino-3-(5-methyl-2-(trifluoromethyl)phenyl)thiazolidin-4-one (prepared as in PCT International Application Publication WO 2017040742 A1) and was isolated as an off-white solid (68 mg, 49%).

### (Z)-1-(2-Cyano-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J245)

The title compound was prepared from 2-imino-3-(5-methyl-2-((2,2,2-trifluoroethoxy)methyl)phenyl)thiazolidin-4-one (C117) and was isolated as an off-white solid (71 mg, 47%).

### (Z)-1-(2-Cyano-4-(1-(4-(trifluoromethoxy)phenyl)-1H-1,2,4-triazol-3-yl)phenyl)-3-(3-(2-isopropyl-5-methoxyphenyl)-4-oxothiazolidin-2-ylidene)urea (J246)

The title compound was prepared from 2-imino-3-(2-isopropyl-5-methoxyphenyl)thiazolidin-4-one (C98) and was isolated as an off-white solid (70 mg, 51%).

### (Z)-1-(2-Cyano-4-(1-(4-(trifluoromethoxy)phenyl)-1H-1,2,4-triazol-3-yl)phenyl)-3-(3-(5-(dimethylamino)-2-propylphenyl)-4-oxothiazolidin-2-ylidene)urea (J247)

The title compound was prepared from 3-(5-(dimethylamino)-2-propylphenyl)-2-iminothiazolidin-4-one (C128) and was isolated as an off-white solid (65 mg, 46%).

### (Z)-1-(2-Cyano-4-(1-(4-(trifluoromethoxy)phenyl)-1H-1,2,4-triazol-3-yl)phenyl)-3-(3-(5-methyl-2-propylphenyl)-4-oxothiazolidin-2-ylidene)urea (J248)

The title compound was prepared from 2-imino-3-(5-methyl-2-propylphenyl)thiazolidin-4-one (prepared as in PCT International Application Publication WO 2016033025 A1) and was isolated as an off-white powder (58 mg, 43%).

### (Z)-1-(2-Cyano-4-(1-(5-(trifluoromethoxy)pyridin-2-yl)-1H-1,2,4-triazol-3-yl)phenyl)-3-(3-(2-isopropyl-5-methylphenyl)-4-oxothiazolidin-2-ylidene)urea (J259)

The title compound was prepared from 2-imino-3-(2-isopropyl-5-methylphenyl)thiazolidin-4-one (prepared as in U.S. Patent Application Publication 20140274688 A1) and was isolated as a yellow semi-solid (70 mg, 78%).

### (Z)-1-(3-(2-Isopropyl-5-methylphenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J260)

The title compound was prepared from 2-imino-3-(2-isopropyl-5-methylphenyl)thiazolidin-4-one (prepared as in U.S. Patent Application Publication 20140274688 A1) and was isolated as a yellow powder (70 mg, 82%).

### (Z)-1-(2-Cyano-4-(1-(5-(trifluoromethoxy)pyridin-2-yl)-1H-1,2,4-triazol-3-yl)phenyl)-3-(3-(5-methyl-2-(3,3,3-trifluoropropoxy)phenyl)-4-oxothiazolidin-2-ylidene)urea (J264)

The title compound was prepared from 2-imino-3-(5-methyl-2-(3,3,3-trifluoropropoxy)phenyl)thiazolidin-4-one (C99) and was isolated as an off-white powder (72 mg, 48%).

### (Z)-1-(2-Cyano-4-(1-(5-(trifluoromethoxy)pyridin-2-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J265)

The title compound was prepared from 2-imino-3-(5-methyl-2-((2,2,2-trifluoroethoxy)methyl)phenyl)thiazolidin-4-one (C117) and was isolated as an off-white powder (74 mg, 50%).

### (Z)-1-(2-Cyano-4-(1-(5-(trifluoromethoxy)pyridin-2-yl)-1H-1,2,4-triazol-3-yl)phenyl)-3-(3-(5-(dimethylamino)-2-isopropylphenyl)-4-oxothiazolidin-2-ylidene)urea (J266)

The title compound was prepared from 3-(5-(dimethylamino)-2-isopropylphenyl)-2-iminothiazolidin-4-one (C126) and was isolated as an off-white powder (59 mg, 42%).

### (Z)-1-(2-Cyano-4-(1-(5-(trifluoromethoxy)pyridin-2-yl)-1H-1,2,4-triazol-3-yl)phenyl)-3-(3-(2-(1-methoxyethyl)-5-methylphenyl)-4-oxothiazolidin-2-ylidene)urea (J267)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white powder (83 mg, 60%).

### (Z)-1-(2-Cyano-4-(1-(5-(trifluoromethoxy)pyridin-2-yl)-1H-1,2,4-triazol-3-yl)phenyl)-3-(3-(2-isopropyl-5-methoxyphenyl)-4-oxothiazolidin-2-ylidene)urea (J268)

The title compound was prepared from 2-imino-3-(2-isopropyl-5-methoxyphenyl)thiazolidin-4-one (C98) and was isolated as an off-white powder (64 mg, 46%).

### (Z)-1-(2-Cyano-4-(1-(5-(trifluoromethoxy)pyridin-2-yl)-1H-1,2,4-triazol-3-yl)phenyl)-3-(3-(5-(dimethylamino)-2-propylphenyl)-4-oxothiazolidin-2-ylidene)urea (J269)

The title compound was prepared from 3-(5-(dimethylamino)-2-propylphenyl)-2-iminothiazolidin-4-one (C128) and was isolated as an off-white powder (67 mg, 48%).

### (Z)-1-(3-(5-Methyl-2-(3,3,3-trifluoropropoxy)phenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J273)

The title compound was prepared from 2-imino-3-(5-methyl-2-(3,3,3-trifluoropropoxy)phenyl)thiazolidin-4-one (C99) and was isolated as an off-white solid (75 mg, 53%).

### (Z)-1-(3-(5-Methyl-2-((2,2,2-trifluoroethoxy)methyl)phenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J274)

The title compound was prepared from 2-imino-3-(5-methyl-2-((2,2,2-trifluoroethoxy)methyl)phenyl)thiazolidin-4-one (C117) and was isolated as an off-white powder (85 mg, 60%).

### (Z)-1-(3-(5-(Dimethylamino)-2-isopropylphenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J275)

The title compound was prepared from 3-(5-(dimethylamino)-2-isopropylphenyl)-2-iminothiazolidin-4-one (C126) and was isolated as an off-white solid (82 mg, 61%).

### (Z)-1-(3-(2-(1-Methoxyethyl)-5-methylphenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J276)

The title compound was prepared from 2-imino-3-(2-(1-methoxyethyl)-5-methylphenyl)-thiazolidin-4-one (prepared as in PCT International Application Publication WO 2017040194 A1) and was isolated as an off-white solid (88 mg, 67%).

### (Z)-1-(3-(2-Isopropyl-5-methoxyphenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J277)

The title compound was prepared from 2-imino-3-(2-isopropyl-5-methoxyphenyl)thiazolidin-4-one (C98) and was isolated as an off-white solid (89 mg, 68%).

### (Z)-1-(3-(5-(Dimethylamino)-2-propylphenyl)-4-oxothiazolidin-2-ylidene)-3-(4-(1-(5-(trifluoromethoxy)pyridin-2-yl)-1H-1,2,4-triazol-3-yl)-2-(trifluoromethyl)phenyl)urea (J278)

The title compound was prepared from 3-(5-(dimethylamino)-2-propylphenyl)-2-iminothiazolidin-4-one (C128) and was isolated as a sticky yellow glass (69 mg, 51.7%).

### (Z)-1-(3-(2-Isopropyl-5-methylphenyl)-4-oxothiazolidin-2-ylidene)-3-(2-(trifluoromethyl)-4-(1-(5-(trifluoromethyl)pyridin-2-yl)-1H-1,2,4-triazol-3-yl)phenyl)urea (J301)

The title compound was prepared from 2-imino-3-(2-isopropyl-5-methylphenyl)thiazolidin-4-one (prepared as in U.S. Patent Application Publication 20140274688 A1) and was isolated as a white film (15 mg, 12%).

### (Z)-1-(3-(5-(Dimethylamino)-2-isopropylphenyl)-4-oxothiazolidin-2-ylidene)-3-(2-(trifluoromethyl)-4-(1-(5-(trifluoromethyl)pyridin-2-yl)-1H-1,2,4-triazol-3-yl)phenyl)urea (J302)

The title compound was prepared from 3-(5-(dimethylamino)-2-isopropylphenyl)-2-iminothiazolidin-4-one (C126) and was isolated as a white waxy powder (54 mg, 40%).

### (Z)-1-(3-(2-(1-Methoxyethyl)-5-methylphenyl)-4-oxothiazolidin-2-ylidene)-3-(2-(trifluoromethyl)-4-(1-(5-(trifluoromethyl)pyridin-2-yl)-1H-1,2,4-triazol-3-yl)phenyl)urea (J303)

The title compound was prepared from 2-imino-3-(2-(1-methoxyethyl)-5-methylphenyl)-thiazolidin-4-one (prepared as in PCT International Application Publication WO 2017040194 A1) and was isolated as a white waxy powder (71 mg, 53%).

### (Z)-1-(3-(5-Methyl-2-((2,2,2-trifluoroethoxy)methyl)phenyl)-4-oxothiazolidin-2-ylidene)-3-(2-(trifluoromethyl)-4-(1-(5-(trifluoromethyl)pyridin-2-yl)-1H-1,2,4-triazol-3-yl)phenyl)urea (J304)

The title compound was prepared from 2-imino-3-(5-methyl-2-((2,2,2-trifluoroethoxy)methyl)phenyl)thiazolidin-4-one (C117) and was isolated as a white waxy powder (63 mg, 44%).

### (Z)-1-(3-(5-Methyl-2-(3,3,3-trifluoropropoxy)phenyl)-4-oxothiazolidin-2-ylidene)-3-(2-(trifluoromethyl)-4-(1-(5-(trifluoromethyl)pyridin-2-yl)-1H-1,2,4-triazol-3-yl)phenyl)urea (J305)

The title compound was prepared from 2-imino-3-(5-methyl-2-(3,3,3-trifluoropropoxy)phenyl)thiazolidin-4-one (C99) and was isolated as a white waxy solid (51 mg, 35%).

### Example 36: Preparation of 2-imino-3-(2-isopropyl-5-methoxyphenyl)thiazolidin-4-one (C98)

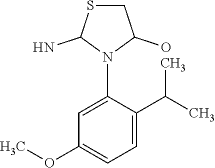 [SMILES: COc1ccc(C(C)C)c(N2C(O)CSC2N)c1]

To a 250 mL round-bottomed flask were added 2-chloro-N-(2-isopropyl-5-methoxyphenyl)acetamide (C155; 4.01 g, 16.6 mmol), acetone (83 mL), and potassium thiocyanate (1.61 g, 16.6 mmol). The reaction mixture was stirred at reflux overnight. The reaction mixture was cooled to room temperature. Cesium carbonate (5.95 g, 18.3 mmol) was added, and the reaction mixture was stirred at room temperature until consumption of starting material. The reaction mixture was filtered over a pad of diatomaceous earth and concentrated under reduced pressure. Purification by column chromatography provided the title compound as a light brown solid (2.6 g, 58%): ¹H NMR (400 MHz, CDCl₃) δ 7.87 (s, 1H), 7.37 (s, 1H), 7.03 (s, 1H), 6.60 (s, 1H), 4.19-3.98 (m, 2H), 3.79 (s, 3H), 2.65 (s, 1H), 1.17 (dd, J=6.9, 4.1 Hz, 6H); ESIMS m/z 265 ([M+H]⁺).

The following compounds were prepared in accordance to the procedure in Example 36.

### 2-Imino-3-(5-methyl-2-(3,3,3-trifluoropropoxy)phenyl)thiazolidin-4-one (C99)

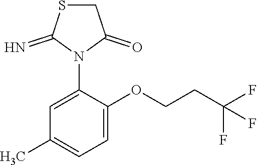 [SMILES: Cc1ccc(OCCC(F)(F)F)c(N2C(=N)SCC2=O)c1]

The title compound was prepared from 2-chloro-N-(5-methyl-2-(3,3,3-trifluoropropoxy)phenyl)acetamide (C156) and was isolated as a dark red thick oil (1.1 g, 51%): ¹H NMR (300 MHz, CDCl₃) δ 7.75 (s, 1H), 7.23 (d, J=8.3 Hz, 1H), 7.10-6.85 (m, 2H), 4.20 (t, J=6.3 Hz, 2H), 4.04 (s, 2H), 2.64-2.44 (m, 2H), 2.33 (s, 3H); ESIMS m/z 319 ([M+H]⁺).

### 3-(3-(Dimethylamino)phenyl)-2-iminothiazolidin-4-one (C100)

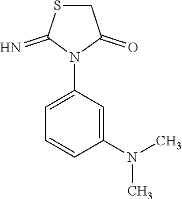 [SMILES: CN(C)c1cccc(N2C(=N)SCC2=O)c1]

The title compound was prepared from 2-chloro-N-(3-(dimethylamino)phenyl)acetamide (C163) and was isolated as short, white crystals (2.61 g, 43%): ¹H NMR (400 MHz, CDCl₃) δ 7.83 (s, 1H), 7.17 (t, J=8.1 Hz, 1H), 6.99 (t, J=2.2 Hz, 1H), 6.72 (dd, J=8.0, 2.0 Hz, 1H), 6.53 (dd, J=8.4, 2.5 Hz, 1H), 3.84 (s, 2H), 2.94 (s, 6H); ESIMS m/z 236 ([M+H]⁺).

### 3-(5-(Dimethylamino)-2-(3,3,3-trifluoropropoxy)phenyl)-2-iminothiazolidin-4-one (C101)

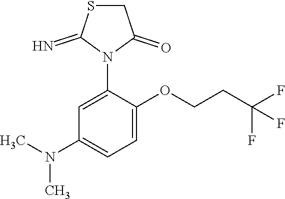 [SMILES: CN(C)c1ccc(OCCC(F)(F)F)c(N2C(=N)SCC2=O)c1]

The title compound was prepared from 2-chloro-N-(5-(dimethylamino)-2-(3,3,3-trifluoropropoxy)phenyl)acetamide (C164) and was isolated as a dark red oil (1.07 g, 41%): ¹H NMR (300 MHz, CDCl₃) δ 6.98 (d, J=9.1 Hz, 1H), 6.79 (dd, J=9.2, 3.0 Hz, 1H), 6.52 (s, 1H), 4.19-4.12 (m, 3H), 4.04 (s, 2H), 2.90 (s, 6H), 2.61-2.39 (m, 2H); ESIMS m/z 348 ([M+H]⁺).

### 3-(4-(Dimethylamino)-2-isopropylphenyl)-2-iminothiazolidin-4-one (C102)

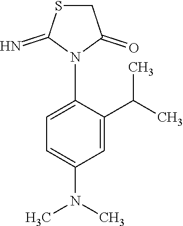 [SMILES: CC(C)c1cc(N(C)C)ccc1N1C(=N)SCC1=O]

The title compound was prepared from 2-chloro-N-(4-(dimethylamino)-2-isopropylphenyl)acetamide (C165) and was isolated as a light orange oil (498 mg, 35%): ¹H NMR (400 MHz, CDCl₃) δ 7.81 (s, 0.5H), 7.04 (s, 0.5H), 6.90 (d, J=8.7 Hz, 1H), 6.78-6.56 (m, 2H), 4.04 (s, 2H), 3.00 (s, 6H), 2.63 (s, 1H), 1.19 (d, J=6.9 Hz, 6H); ESIMS m/z 278 ([M+H]⁺).

### 3-(5-(Dimethylamino)-4-fluoro-2-isopropylphenyl)-2-iminothiazolidin-4-one (C103)

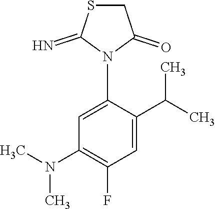 [SMILES: CC(C)c1cc(F)c(N(C)C)cc1N1C(=N)SCC1=O]

The title compound was prepared from 2-chloro-N-(5-(dimethylamino)-4-fluoro-2-isopropylphenyl)acetamide (C171) and was isolated as a dark orange oil (1.85 g, 74%): ¹H NMR (300 MHz, CDCl₃) δ 7.86 (s, 1H), 7.06 (d, J=15.1 Hz, 1H), 6.55 (d, J=9.0 Hz, 1H), 4.10 (d, J=4.5 Hz, 2H), 2.83 (s, 6H), 2.60 (s, 1H), 1.15 (dd, J=7.0, 4.1 Hz, 6H); ESIMS m/z 296 ([M+H]⁺).

### 3-(2,5-Diisopropylphenyl)-2-iminothiazolidin-4-one (C104)

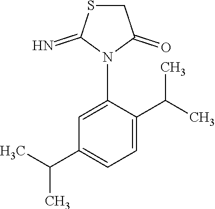 [SMILES: CC(C)c1ccc(C(C)C)c(N2C(=N)SCC2=O)c1]

The title compound was prepared from 2-chloro-N-(2,5-diisopropylphenyl)acetamide (C186) and was isolated as a white solid (2.9 g, 67%): ¹H NMR (300 MHz, CDCl₃) δ 7.47-7.28 (m, 2H), 7.01-6.84 (m, 1H), 4.11 (d, J=0.9 Hz, 1H), 4.06 (d, J=1.2 Hz, 1H), 2.91 (p, J=7.1 Hz, 1H), 2.66 (dq, J=16.4, 6.9 Hz, 1H), 1.25 (d, J=6.9 Hz, 6H), 1.22-1.13 (m, 6H); ESIMS m/z 277 ([M+H]⁺).

### 3-(5-Hydroxy-2-isopropylphenyl)-2-iminothiazolidin-4-one (C105)

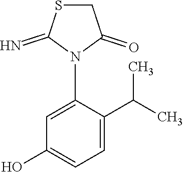 [SMILES: CC(C)c1ccc(O)cc1N1C(=N)SCC1=O]

The title compound was prepared from 2-chloro-N-(5-hydroxy-2-isopropylphenyl)acetamide (C187) and was isolated as a white solid (160 mg, 21%): ¹H NMR (400 MHz, CDCl₃) δ 7.77 (s, 1H), 6.76 (s, 2H), 6.40 (s, 2H), 4.11 (s, 2H), 2.59 (p, J=6.8 Hz, 1H), 1.16 (t, J=7.2 Hz, 6H); ESIMS m/z 251 ([M+H]⁺).

### 2-Imino-3-(2-(piperidin-1-yl)phenyl)thiazolidin-4-one (C106)

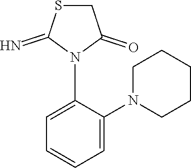 [SMILES: N=C1SCC(=O)N1c1ccccc1N1CCCCC1]

The title compound was prepared from 2-chloro-N-(5-methyl-2-(piperidin-1-yl)phenyl)acetamide (C188) and was isolated as a white foam (450 mg, 14%): ¹H NMR (400 MHz, CDCl₃) δ 7.76 (s, 1H), 7.46-7.37 (m, 1H), 7.26-7.20 (m, 1H), 7.20-7.12 (m, 2H), 4.05 (s, 2H), 2.80 (d, J=6.4 Hz, 4H), 1.77-1.47 (m, 6H); ESIMS m/z 276 ([M+H]⁺).

### 2-Imino-3-(5-methyl-2-(4,4,4-trifluorobutoxy)phenyl)thiazolidin-4-one (C107)

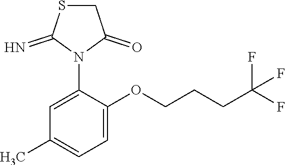 [SMILES: Cc1ccc(OCCCC(F)(F)F)c(N2C(=N)SCC2=O)c1]

The title compound was prepared from 2-chloro-N-(5-methyl-2-(4,4,4-trifluorobutoxy)phenyl)acetamide (C177) and was isolated as a red solid (1.81 g, 64%): ¹H NMR (400 MHz, CDCl₃) δ 7.78 (s, 1H), 7.20 (d, J=8.5 Hz, 1H), 7.14-6.81 (m, 2H), 4.09-3.94 (m, 4H), 2.32 (s, 3H), 2.21 (dtdd, J=13.6, 10.9, 7.4, 4.9 Hz, 2H), 1.98 (m, 2H); ¹⁹F NMR (376 MHz, CDCl₃) δ−66.14 (d, J=38.1 Hz), −66.39 (d, J=30.3 Hz); ESIMS m/z 334 ([M−H]⁺).

### 3-(2-Imino-4-oxothiazolidin-3-yl)-4-isopropylbenzonitrile (C108)

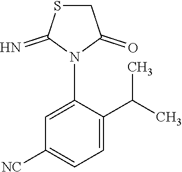 [SMILES: CC(C)c1ccc(C#N)cc1N1C(=N)SCC1=O]

The title compound was prepared from 2-chloro-N-(5-cyano-2-isopropylphenyl)acetamide (C154) and was isolated as a red solid (2.5 g, 57%): mp 174-178° C.; ¹H NMR (400 MHz, DMSO-d₆) δ 9.34 (s, 1H), 7.90 (dd, J=1.6, 8.4 Hz, 1H), 7.74 (s, 1H), 7.68 (d, J=8.4 Hz, 1H), 4.32-4.28 (m, 1H), 4.16-4.12 (m, 1H), 2.81-2.77 (m, 1H), 1.14-1.09 (m, 6H); ESIMS m/z 260 ([M+H]⁺).

### 3-(5-Acetyl-2-isopropylphenyl)-2-iminothiazolidin-4-one (C109)

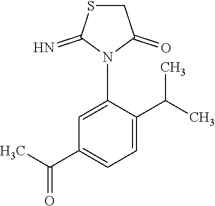 [SMILES: CC(=O)c1ccc(C(C)C)c(N2C(=N)SCC2=O)c1]

The title compound was prepared from N-(5-acetyl-2-isopropylphenyl)-2-chloroacetamide (C181) and was isolated as a pale red solid (1.38 g, 50%): mp 114-118° C.; ¹H NMR (400 MHz, DMSO-d₆) δ 9.27 (s, 1H), 8.00 (d, J=8.0 Hz, 1H), 7.72 (s, 1H), 7.61 (d, J=8.4 Hz, 1H), 4.30-4.15 (m, 2H), 2.81-2.74 (m, 1H), 2.56 (s, 3H), 1.15-1.09 (m, 6H); ESIMS m/z 277 ([M+H]⁺).

### 2-Imino-3-(5-methoxy-2-(3,3,3-trifluoropropoxy)phenyl)thiazolidin-4-one (C110)

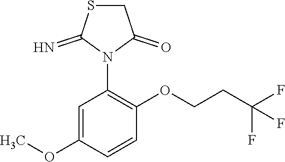 [SMILES: COc1ccc(OCCC(F)(F)F)c(N2C(=N)SCC2=O)c1]

The title compound was prepared from 2-chloro-N-(5-methoxy-2-(3,3,3-trifluoropropoxy)phenyl)acetamide (C182) and was isolated as a pale red solid (2.4 g, 44%): mp 89-93° C.; ¹H NMR (400 MHz, DMSO-d₆) δ 9.13 (s, 1H), 7.16 (d, J=9.2 Hz, 1H), 6.98 (dd, J=2.8, 9.2 Hz, 1H), 6.80 (d, J=2.8 Hz, 1H), 4.16-4.01 (m, 4H), 3.72 (s, 3H), 2.69-2.58 (m, 2H); ESIMS m/z 335 ([M+H]⁺).

### 3-(2-Imino-4-oxothiazolidin-3-yl)-4-(3,3,3-trifluoropropoxy)benzonitrile (C111)

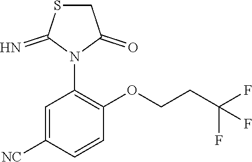 [SMILES: N#Cc1ccc(OCCC(F)(F)F)c(N2C(=N)SCC2=O)c1]

The title compound was prepared from 2-chloro-N-(5-cyano-2-(3,3,3-trifluoropropoxy)phenyl)acetamide (C184) and was isolated as a pale red solid (1.2 g, 69%): ¹H NMR (400 MHz, DMSO-d₆) δ 9.25 (s, 1H), 7.95 (dd, J=2.0, 8.8 Hz, 1H), 7.79 (d, J=2.0 Hz, 1H), 7.41 (d, J=8.8 Hz, 1H), 4.34-4.27 (m, 2H), 4.17-4.04 (m, 2H), 2.77-2.66 (m, 2H); ESIMS m/z 330 ([M+H]⁺).

### Methyl 3-(2-imino-4-oxothiazolidin-3-yl)-4-isopropylbenzoate (C112)

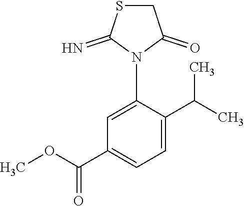 [SMILES: COC(=O)c1ccc(C(C)C)c(N2C(=N)SCC2=O)c1]

The title compound was prepared from methyl 3-(2-chloroacetamido)-4-isopropylbenzoate (C185) and was isolated as a pale yellow solid (1.2 g, 30%): mp 159-164° C.; ¹H NMR (400 MHz, DMSO-d₆) δ 9.27 (s, 1H), 7.99 (dd, J=1.6, 8.4 Hz, 1H), 7.73 (d, J=1.2 Hz, 1H), 7.62 (d, J=8.4 Hz, 1H), 4.28-4.14 (m, 2H), 3.85 (s, 3H), 2.85-2.76 (m, 1H), 1.14-1.10 (m, 6H); ESIMS m/z 293 ([M+H]⁺).

### 3-(5-Chloro-2-(trifluoromethyl)phenyl)-2-iminothiazolidin-4-one (C5)

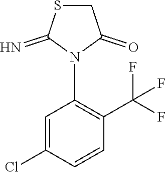 [SMILES: N=C1SCC(=O)N1c1cc(Cl)ccc1C(F)(F)F]

The title compound was prepared from 2-chloro-N-(5-chloro-2-(trifluoromethyl)phenyl)acetamide (C159) and was isolated as a yellow solid (4.2 g, 72%): mp 156-157° C.; ¹H NMR (500 MHz, CDCl₃) δ 7.94 (s, 1H), 7.75 (d, J=8.4 Hz, 1H), 7.62-7.55 (m, 1H), 7.35 (d, J=1.9 Hz, 1H), 4.19-4.03 (m, 2H); ¹⁹F NMR (471 MHz, CDCl₃) δ −61.49; ESIMS m/z 295 ([M+H]⁺).

### 2-Imino-3-(5-methoxy-2-propylphenyl)thiazolidin-4-one (C113)

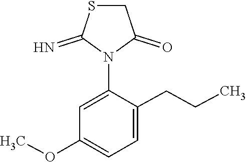 [SMILES: CCCc1ccc(OC)cc1N1C(=N)SCC1=O]

The title compound was prepared from 2-chloro-N-(5-methoxy-2-propylphenyl)acetamide (C160) and was isolated as a pale yellow solid (3.5 g, 45%): mp 68-72° C.; ¹H NMR (400 MHz, DMSO-d₆) δ 9.21 (s, 1H), 7.24 (d, J=7.8 Hz, 1H), 6.95 (dd, J=1.6, 8.0 Hz, 1H), 6.71 (d, J=2.0 Hz, 1H), 4.26-4.10 (m, 2H), 3.73 (s, 3H), 2.26 (t, J=7.6 Hz, 2H), 1.47-1.41 (m, 2H), 0.83 (t, J=7.2 Hz, 3H); ESIMS m/z 265 ([M+H]⁺).

### 3-(2-Butyl-5-methylphenyl)-2-iminothiazolidin-4-one (C114)

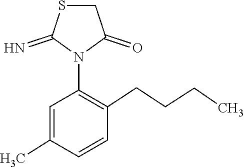 [SMILES: CCCCc1ccc(C)cc1N1C(=N)SCC1=O]

The title compound was prepared from N-(2-butyl-5-methylphenyl)-2-chloroacetamide (C161) and was isolated as an orange gummy liquid (0.6 g, 38%): ¹H NMR (400 MHz, DMSO-d₆) δ 9.22 (s, 1H), 7.23-7.16 (m, 2H), 6.91 (s, 1H), 4.24-4.12 (m, 2H), 2.32-2.28 (m, 5H), 1.44-1.37 (m, 2H), 1.29-1.21 (m, 2H), 0.84 (t, J=7.2 Hz, 3H); ¹³C NMR (101 MHz, DMSO-d₆) δ 171.73, 157.82, 137.30, 135.87, 133.89, 129.66, 129.54, 129.42, 33.67, 31.60, 29.80, 21.91, 20.25, 13.67; ESIMS m/z 263 ([M+H]⁺).

### 3-(2-(sec-Butyl)-5-methylphenyl)-2-iminothiazolidin-4-one (C115)

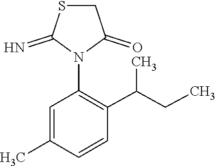 [SMILES: CCC(C)c1ccc(C)cc1N1C(=N)SCC1=O]

The title compound was prepared from N-(2-(sec-butyl)-5-methylphenyl)-2-chloroacetamide (C196) and was isolated as a brown gummy liquid (0.35 g, 12%): ¹H NMR (400 MHz, DMSO-d₆) δ 9.19 (d, J=4.4 Hz, 1H), 7.27-7.20 (m, 2H), 6.88 (s, 1H), 4.27-4.12 (m, 2H), 2.38-2.28 (m, 4H), 1.48-1.43 (m, 2H), 1.04 (dd, J=6.8, 11.2 Hz, 3H), 0.71-0.65 (m, 3H); ESIMS m/z 263 ([M+H]⁺).

### 3-(2-Butyl-5-methoxyphenyl)-2-iminothiazolidin-4-one (C116)

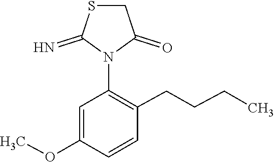 [SMILES: CCCCc1ccc(OC)cc1N1C(=N)SCC1=O]

The title compound was prepared from N-(2-butyl-5-methoxyphenyl)-2-chloroacetamide (C162) and was isolated as a red semi-solid (1.2 g, 24%): ¹H NMR (400 MHz, DMSO-d₆) δ 9.21 (br s, 1H), 7.23 (d, J=8.4 Hz, 1H), 6.95 (d, J=7.6 Hz, 1H), 6.71 (s, 1H), 4.23-4.11 (m, 2H), 3.72 (s, 3H), 2.28 (t, J=7.2 Hz, 2H), 1.42-1.36 (m, 2H), 1.28-1.21 (m, 2H), 0.88-0.81 (s, 3H); ¹³C NMR (75 MHz, CDCl₃) δ 170.75, 160.54, 158.58, 132.74, 132.51, 131.02, 116.29, 113.76, 55.35, 33.77, 32.06, 29.89, 22.42, 13.75; ESIMS m/z 279 ([M+H]⁺).

### 2-Imino-3-(5-methyl-2-((2,2,2-trifluoroethoxy)methyl)phenyl)thiazolidin-4-one (C117)

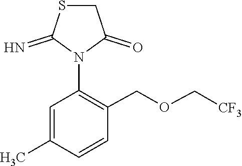 [SMILES: Cc1ccc(COCC(F)(F)F)c(N2C(=N)SCC2=O)c1]

The title compound was prepared from 2-chloro-N-(5-methyl-2-((2,2,2-trifluoroethoxy)methyl)phenyl)acetamide (C166) and was isolated as a pale yellow solid (2.7 g, 62%): mp 70-75° C.; ¹H NMR (400 MHz, DMSO-d₆) δ 9.23 (s, 1H), 7.40 (d, J=8.0 Hz, 1H), 7.29 (d, J=8.0 Hz, 1H), 7.03 (s, 1H), 4.49-4.42 (m, 2H), 4.14 (s, 2H), 3.98-3.90 (m, 2H), 2.33 (s, 3H); ¹⁹F NMR (376 MHz, DMSO-d₆) δ−72.79; ESIMS m/z 319 ([M+H]⁺).

### 3-(2-(1-Ethoxyethyl)-5-methylphenyl)-2-iminothiazolidin-4-one (C118)

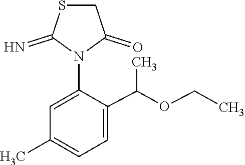 [SMILES: CCOC(C)c1ccc(C)cc1N1C(=N)SCC1=O]

The title compound was prepared from 2-chloro-N-(2-(1-ethoxyethyl)-5-methylphenyl)acetamide (C169) and was isolated as a pale brown solid (2.3 g, 54%), as a mixture of isomers: mp 135-139° C.; ¹H NMR (400 MHz, DMSO-d₆) δ 9.27-9.23 (m, 1H), 7.39 (d, J=8.0 Hz, 1H), 7.28 (d, J=7.6 Hz, 1H), 6.93 (s, 1H), 4.28-4.20 (m, 2H), 4.17-4.12 (m, 1H), 3.21-3.09 (m, 2H), 2.31 (s, 3H), 1.23-1.17 (m, 3H), 1.00 (t, J=6.8 Hz, 3H); ESIMS m/z 279 ([M+H]⁺).

### 2-Imino-3-(5-methyl-2-propoxyphenyl)thiazolidin-4-one (C119)

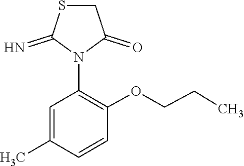 [SMILES: CCCOc1ccc(C)cc1N1C(=N)SCC1=O]

The title compound was prepared from 2-chloro-N-(5-methyl-2-propoxyphenyl)acetamide (C170) and was isolated as a yellow solid (1.7 g, 40%): mp 78-82° C.; ¹H NMR (400 MHz, DMSO-d₆) δ 9.11 (s, 1H), 7.17 (dd, J=1.6, 8.4 Hz, 1H), 7.02 (d, J=8.8 Hz, 1H), 6.94 (d, J=2.0 Hz, 1H), 4.18-4.07 (m, 2H), 3.87 (t, J=8.0 Hz, 2H), 2.25 (s, 3H), 1.65-1.56 (m, 2H), 0.88 (t, J=7.6 Hz, 3H); ESIMS m/z 265 ([M+H]⁺).

### 3-(2-(Ethoxymethyl)-5-methylphenyl)-2-iminothiazolidin-4-one (C120)

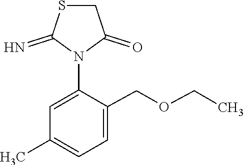 [SMILES: CCOCc1ccc(C)cc1N1C(=N)SCC1=O]

The title compound was prepared from 2-chloro-N-(2-(ethoxymethyl)-5-methylphenyl)acetamide (C172) and was isolated as a cream solid (1.5 g, 35%): mp 58-61° C.; ¹H NMR (400 MHz, DMSO-d₆) δ 9.22 (s, 1H), 7.36 (d, J=7.6 Hz, 1H), 7.24 (d, J=7.6 Hz, 1H), 6.98 (s, 1H), 4.29-4.20 (m, 2H), 4.15 (s, 2H), 3.36-3.31 (m, 2H), 2.31 (s, 3H), 1.08 (t, J=6.8 Hz, 3H); ESIMS m/z 265 ([M+H]⁺).

### 2-Imino-3-(5-methyl-2-(2,2,2-trifluoro-1-methoxyethyl)phenyl)thiazolidin-4-one (C121)

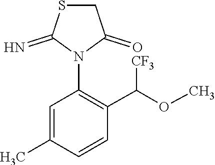 [SMILES: COC(c1ccc(C)cc1N1C(=N)SCC1=O)C(F)(F)F]

The title compound was prepared from 2-chloro-N-(5-methyl-2-(2,2,2-trifluoro-1-methoxyethyl)phenyl)acetamide (C173) and was isolated as a cream solid (2.9 g, 52%): mp 129-132° C.; ¹H NMR (400 MHz, DMSO-d₆) δ 9.37 (s, 1H), 7.48 (d, J=8.0 Hz, 1H), 7.38 (d, J=8.4 Hz, 1H), 7.11 (s, 1H), 4.68 (q, J=6.4 Hz, 1H), 4.25-4.11 (m, 2H), 3.25 (s, 3H), 2.36 (s, 3H); ¹⁹F NMR (376 MHz, DMSO-d₆) δ−75.26; ESIMS m/z 319 ([M+H]⁺).

### 2-Imino-3-(2-isobutyl-5-methylphenyl) thiazolidin-4-one (C122)

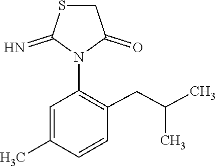 [SMILES: Cc1ccc(CC(C)C)c(N2C(=N)SCC2=O)c1]

The title compound was prepared from 2-chloro-N-(2-isobutyl-5-methylphenyl)acetamide (C179) and was isolated as a pale yellow semi-solid (1.4 g, 63%): ¹H NMR (400 MHz, DMSO-d₆) δ 9.20 (s, 1H), 7.18 (s, 2H), 6.91 (s, 1H), 4.25-4.11 (m, 2H), 2.29 (s, 3H), 2.20 (d, J=7.6 Hz, 2H), 1.76-1.69 (m, 1H), 0.83-0.77 (m, 6H); ESIMS m/z 263 ([M+H]⁺).

### 3-(2-(sec-Butyl)-5-methoxyphenyl)-2-iminothiazolidin-4-one (C123)

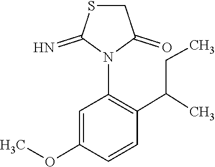 [SMILES: CCC(C)c1ccc(OC)cc1N1C(=N)SCC1=O]

The title compound was prepared from N-(2-(sec-butyl)-5-methoxyphenyl)-2-chloroacetamide (C157) and was isolated as an off-white solid (0.5 g, 15%): mp 115-117° C.; ¹H NMR (400 MHz, DMSO-d₆) δ 9.20 (d, J=5.2 Hz, 1H), 7.27 (d, J=8.4 Hz, 1H), 6.99 (dd, J=2.8, 8.8 Hz, 1H), 6.68 (d, J=2.0 Hz, 1H), 4.29-4.09 (m, 2H), 3.73 (s, 3H), 2.38-2.31 (m, 1H), 1.58-1.39 (m, 2H), 1.05 (dd, J=6.8, 10.8 Hz, 3H), 0.75-0.63 (m, 3H); ESIMS m/z 279 ([M+H]⁺).

### 2-Imino-3-(2-(1-methoxyethyl)phenyl)thiazolidin-4-one (C124)

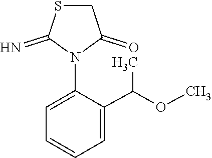 [SMILES: COC(C)c1ccccc1N1C(=N)SCC1=O]

The title compound was prepared from 2-chloro-N-(2-(1-methoxyethyl)phenyl)acetamide (C183) and was isolated as an orange oil (400 mg, 6.7%): ¹H NMR (300 MHz, CDCl₃) δ 7.70-7.58 (m, 1H), 7.55 (ddt, J=7.7, 6.1, 1.6 Hz, 1H), 7.49-7.38 (m, 1H), 7.10 (ddd, J=11.5, 7.9, 1.2 Hz, 1H), 5.51 (s, 1H), 4.24-4.15 (m, 1H), 4.04-3.93 (m, 1H), 3.79-3.65 (m, 1H), 3.24-3.02 (m, 3H), 1.45-1.31 (m, 3H).

### 3-(5-Ethoxy-2-propylphenyl)-2-iminothiazolidin-4-one (C125)

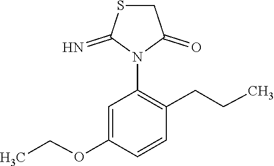 [SMILES: CCCc1ccc(OCC)cc1N1C(=N)SCC1=O]

The title compound was prepared from 2-chloro-N-(5-ethoxy-2-propylphenyl)acetamide (C200) and was isolated as a red oil (423 mg, 32%): ¹H NMR (400 MHz, CDCl₃) δ 7.85 (s, 1H), 7.29 (s, 1H), 6.96 (d, J=8.7 Hz, 1H), 6.62 (d, J=2.7 Hz, 1H), 4.08 (d, J=2.0 Hz, 2H), 4.00 (q, J=7.0 Hz, 2H), 2.33 (t, J=7.8 Hz, 2H), 1.63-1.48 (m, 2H), 1.39 (t, J=7.0 Hz, 3H), 0.91 (t, J=7.3 Hz, 3H); HRMS-ESI (m/z) [M+H]⁺ calcd for C₁₄H₁₈N₂O₂S, 279.1162; found, 279.1164.

### Example 37: Preparation of 3-(5-(dimethylamino)-2-isopropylphenyl)-2-iminothiazolidin-4-one (C126)

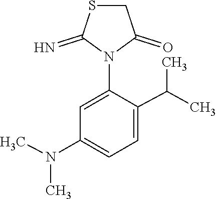 [SMILES: CC(C)c1ccc(N(C)C)cc1N1C(=N)SCC1=O]

To a solution of 2-chloro-N-(5-(dimethylamino)-2-isopropylphenyl)acetamide (C158; 0.8 g, 3.14 mmol) in acetone (12.5 mL) was added potassium thiocyanate (0.38 g, 3.93 mmol), and the reaction mixture was stirred at reflux for 8 h. The reaction mixture was cooled to room temperature. Potassium carbonate (0.022 g, 0.16 mmol) was added, and the reaction mixture was stirred at room temperature for 1 h. The reaction mixture was filtered through a pad of diatomaceous earth, and the filtrate was concentrated under reduced pressure. Purification by column chromatography eluting with 0-40% EtOAc-hexanes afforded the title compound as a sticky, brown solid (0.30 g, 34%): ¹H NMR (400 MHz, DMSO-d₆) δ 9.16 (s, 1H), 7.21 (d, J=8.8 Hz, 1H), 6.80 (dd, J=2.4, 8.8 Hz, 1H), 6.39 (d, J=3.2 Hz, 1H), 4.25-4.10 (m, 2H), 2.85 (s, 6H), 2.58-2.53 (m, 1H), 1.07-1.02 (m, 6H); ESIMS m/z 278 ([M+H]⁺).

The following compounds were prepared in accordance to the procedure in Example 37.

### 3-(5-(Dimethylamino)-2-(trifluoromethyl)phenyl)-2-iminothiazolidin-4-one (C127)

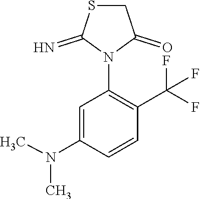 [SMILES: CN(C)c1ccc(C(F)(F)F)c(N2C(=N)SCC2=O)c1]

The title compound was prepared from 2-chloro-N-(5-(dimethylamino)-2-(trifluoromethyl)phenyl)acetamide (C174) and was isolated as a pale brown solid (3.1 g, 57%): mp 175-177° C.; ¹H NMR (400 MHz, DMSO-d₆) δ 9.25 (s, 1H), 7.53 (d, J=9.2 Hz, 1H), 6.82 (dd, J=2.0, 8.8 Hz, 1H), 6.65 (d, J=2.4 Hz, 1H), 4.20-4.09 (m, 2H), 2.98 (s, 6H); ¹⁹F NMR (376 MHz, DMSO-d₆) δ−58.15; ESIMS m/z 304 ([M+H]⁺).

### 3-(5-(Dimethylamino)-2-propylphenyl)-2-iminothiazolidin-4-one (C128)

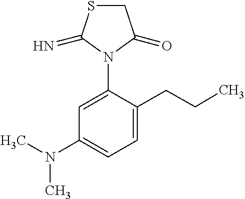 [SMILES: CCCc1ccc(N(C)C)cc1N1C(=N)SCC1=O]

The title compound was prepared from 2-chloro-N-(5-(dimethylamino)-2-propylphenyl)acetamide (C175) and was isolated as a brown gummy liquid (3.4 g, 62%): ¹H NMR (300 MHz, DMSO-d₆) δ 9.22 (s, 1H), 7.12 (d, J=8.7 Hz, 1H), 6.74 (dd, J=2.7, 8.4 Hz, 1H), 6.42 (d, J=2.7 Hz, 1H), 4.24-4.08 (m, 2H), 2.85 (s, 6H), 2.22 (t, J=7.5 Hz, 2H), 1.46-1.36 (m, 2H), 0.82 (t, J=6.9 Hz, 3H); ¹³C NMR (75 MHz, DMSO-d₆) δ 171.68, 157.83, 149.45, 134.63, 129.77, 127.31, 113.14, 113.03, 40.34, 33.64, 31.91, 22.79, 13.97; ESIMS m/z 278 ([M+H]⁺).

### 3-(5-(Dimethylamino)-2-(2,2,2-trifluoroethoxy)phenyl)-2-iminothiazolidin-4-one (C129)

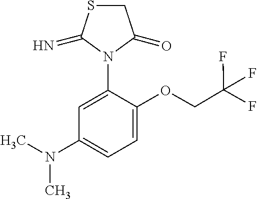 [SMILES: CN(C)c1ccc(OCC(F)(F)F)c(N2C(=N)SCC2=O)c1]

The title compound was prepared from 2-chloro-N-(5-(dimethylamino)-2-(2,2,2-trifluoroethoxy)phenyl)acetamide (C176) and was isolated as a pale orange gummy liquid (2.1 g, 46%): ¹H NMR (400 MHz, DMSO-d₆) δ 9.29 (s, 1H), 7.16 (d, J=8.8 Hz, 1H), 6.79 (d, J=7.6 Hz, 1H), 6.58 (d, J=2.4 Hz, 1H), 4.51 (q, J=8.8 Hz, 2H), 4.17-4.05 (m, 2H), 2.84 (s, 6H); ¹³C NMR (101 MHz, DMSO-d₆) δ 171.21, 157.44, 146.95, 144.29, 125.12, 122.35, 117.06, 114.30, 113.85, 66.82, 54.90, 33.56; ¹⁹F NMR (376 MHz, DMSO-d₆) δ−73.11; ESIMS m/z 334 ([M+H]⁺).

### 3-(5-(Diethylamino)-2-isopropylphenyl)-2-iminothiazolidin-4-one (C130)

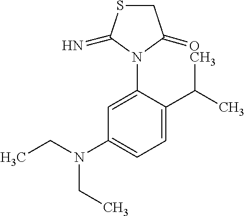 [SMILES: CCN(CC)c1ccc(C(C)C)c(N2C(=N)SCC2=O)c1]

The title compound was prepared from 2-chloro-N-(5-(diethylamino)-2-isopropylphenyl)acetamide (C167) and was isolated as a red gummy liquid (2.4 g, 59%): ¹H NMR (400 MHz, DMSO-d₆) δ 9.14 (s, 1H), 7.17 (d, J=8.8 Hz, 1H), 6.70 (dd, J=2.8, 8.8 Hz, 1H), 6.29 (d, J=2.8 Hz, 1H), 4.24-4.10 (m, 2H), 3.28 (q, J=6.8 Hz, 4H), 2.57-2.52 (m, 1H), 1.10-1.02 (m, 12H); ESIMS m/z 306 ([M+H]⁺).

### 3-(5-(Dipropylamino)-2-isopropylphenyl)-2-iminothiazolidin-4-one (C131)

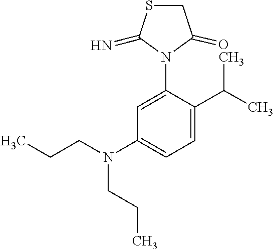 [SMILES: CCCN(CCC)c1ccc(C(C)C)c(N2C(=N)SCC2=O)c1]

The title compound was prepared from 2-chloro-N-(5-(dipropylamino)-2-isopropylphenyl)acetamide (C168) and was isolated as a pale red liquid (1.2 g, 28%): ¹H NMR (400 MHz, DMSO-d₆) δ 9.13 (s, 1H), 7.16 (d, J=8.8 Hz, 1H), 6.67 (dd, J=2.8, 8.8 Hz, 1H), 6.27 (d, J=2.4 Hz, 1H), 4.22-4.10 (m, 2H), 3.18 (t, J=7.2 Hz, 4H), 2.57-2.51 (m, 1H), 1.55-1.45 (m, 4H), 1.09-1.01 (m, 6H), 0.87 (t, J=7.2 Hz, 6H); ESIMS m/z 334 ([M+H]⁺).

### tert-Butyl (3-(2-imino-4-oxothiazolidin-3-yl)-4-isopropylphenyl)(methyl)-carbamate (C132)

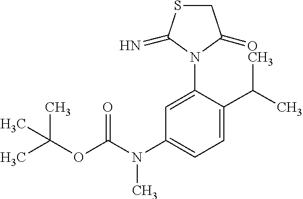 [SMILES: CC(C)c1ccc(N(C)C(=O)OC(C)(C)C)cc1N1C(=N)SCC1=O]

The title compound was prepared from tert-butyl (3-(2-chloroacetamido)-4-isopropylphenyl)(methyl)carbamate (C189) and was isolated as an off-white solid (4.0 g, 74%): ¹H NMR (400 MHz, DMSO-d₆) δ 9.24 (s, 1H), 7.40-7.32 (m, 2H), 7.05 (d, J=1.2 Hz, 1H), 4.28-4.12 (m, 2H), 3.18 (s, 3H), 2.69-2.64 (m, 1H), 1.41 (s, 9H), 1.11-1.07 (m, 6H); ESIMS m/z 364 ([M+H]⁺).

### tert-Butyl ethyl(3-(2-imino-4-oxothiazolidin-3-yl)-4-isopropylphenyl)carbamate (C133)

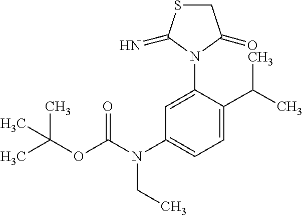 [SMILES: CCN(C(=O)OC(C)(C)C)c1ccc(C(C)C)c(N2C(=N)SCC2=O)c1]

The title compound was prepared from tert-butyl (3-(2-chloroacetamido)-4-isopropylphenyl)(ethyl)carbamate (C190) and was isolated as an off-white solid (4.5 g, 70%): ¹H NMR (400 MHz, DMSO-d₆) δ 9.21 (s, 1H), 7.39 (d, J=8.8 Hz, 1H), 7.28 (dd, J=1.6, 8.8 Hz, 1H), 6.99 (d, J=2.0 Hz, 1H), 4.27-4.12 (m, 2H), 3.58 (q, J=6.8 Hz, 2H), 2.70-2.65 (m, 1H), 1.39 (s, 9H), 1.12-1.05 (m, 9H); ESIMS m/z 378 ([M+H]⁺).

### tert-Butyl (3-(2-imino-4-oxothiazolidin-3-yl)-4-isopropylphenyl)(propyl)-carbamate (C134)

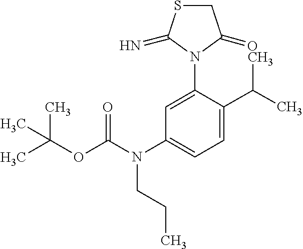 [SMILES: CCCN(C(=O)OC(C)(C)C)c1ccc(C(C)C)c(N2C(=N)SCC2=O)c1]

The title compound was prepared from tert-butyl (3-(2-chloroacetamido)-4-isopropylphenyl)(propyl)carbamate (C191) and was isolated as an off-white solid (6.0 g, 56%): ¹H NMR (400 MHz, DMSO-d₆) δ 9.22 (s, 1H), 7.39 (d, J=8.4 Hz, 1H), 7.28 (dd, J=2.0, 8.4 Hz, 1H), 6.99 (d, J=2.0 Hz, 1H), 4.26-4.12 (m, 2H), 3.52 (t, J=7.2 Hz, 2H), 2.72-2.62 (m, 1H), 1.49-1.42 (m, 2H), 1.39 (s, 9H), 1.12-1.07 (m, 6H), 0.81 (t, J=7.2 Hz, 3H); ESIMS m/z 392 ([M+H]⁺).

### 2-Imino-3-(5-methyl-2-nitrophenyl)thiazolidin-4-one (C136)

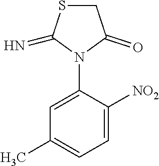 [SMILES: Cc1ccc([N+](=O)[O-])c(N2C(=N)SCC2=O)c1]

The title compound was prepared from 2-chloro-N-(5-methyl-2-nitrophenyl)acetamide (C178) and was isolated as an orange solid (4.72 g, 61%): ¹H NMR (300 MHz, CDCl₃) δ 8.15 (d, J=8.4 Hz, 1H), 7.83 (s, 1H), 7.42 (dd, J=8.5, 1.6 Hz, 1H), 7.23 (d, J=1.9 Hz, 1H), 4.14 (q, J=16.9 Hz, 2H), 2.50 (s, 3H); ESIMS m/z 252 ([M+H]⁺).

### 3-(2,5-Bis(trifluoromethyl)phenyl)-2-iminothiazolidin-4-one (C137)

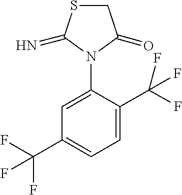 [SMILES: N=C1SCC(=O)N1c1cc(C(F)(F)F)ccc1C(F)(F)F]

The title compound was prepared from N-(2,5-bis(trifluoromethyl)phenyl)-2-chloroacetamide (C180) and was isolated as an off-white solid (3.76 g, 54%): mp 138-142° C.; ¹H NMR (300 MHz, DMSO-d₆) δ 9.42 (s, 1H), 8.17-8.09 (m, 3H), 4.31-4.10 (m, 2H); ¹⁹F NMR (376 MHz, DMSO-d₆) δ−60.79, −61.77; ESIMS m/z 329 ([M+H]⁺).

### N-(3-(2-Imino-4-oxothiazolidin-3-yl)-4-isopropylphenyl)-N-methylacetamide (C138)

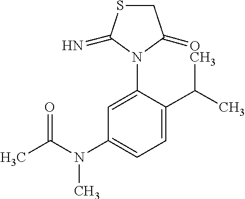 [SMILES: CC(=O)N(C)c1ccc(C(C)C)c(N2C(=N)SCC2=O)c1]

The title compound was prepared from 2-chloro-N-(2-isopropyl-5-(N-methylacetamido)phenyl)acetamide (C193) and was isolated as an off-white solid (1.56 g, 76%): ¹H NMR (400 MHz, CDCl₃) δ 7.87 (s, 1H), 7.53-7.47 (m, 1H), 7.28 (s, 1H), 6.95 (s, 1H), 4.11 (d, J=3.9 Hz, 2H), 3.27 (s, 3H), 2.76 (s, 1H), 1.93 (s, 3H), 1.22 (t, J=6.9 Hz, 6H); ESIMS m/z 306 ([M+H]⁺).

### N-Ethyl-N-(3-(2-imino-4-oxothiazolidin-3-yl)-4-isopropylphenyl)acetamide (C139)

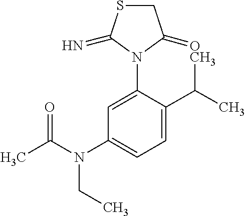 [SMILES: CCN(C(C)=O)c1ccc(C(C)C)c(N2C(=N)SCC2=O)c1]

The title compound was prepared from 2-chloro-N-(5-(N-ethylacetamido)-2-isopropylphenyl)acetamide (C194) and was isolated as an off-white solid (1.41 g, 76%): ¹H NMR (400 MHz, CDCl₃) δ 7.86 (s, 1H), 7.50 (d, J=8.4 Hz, 1H), 7.26 (s, 1H), 6.92 (d, J=2.3 Hz, 1H), 4.18-4.05 (m, 2H), 3.73 (q, J=7.1 Hz, 2H), 2.77 (s, 1H), 1.87 (s, 3H), 1.22 (t, J=6.5 Hz, 6H), 1.13 (t, J=7.1 Hz, 3H); ESIMS m/z 320 ([M+H]⁺).

### Example 38. Preparation of 2-imino-3-(5-methoxy-2-(trifluoromethyl)phenyl)thiazolidin-4-one (C140)

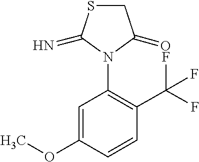 [SMILES: COc1ccc(C(F)(F)F)c(N2C(=N)SCC2=O)c1]

To a solution of 2-chloro-N-(5-methoxy-2-(trifluoromethyl)phenyl)acetamide (C201; 1.01 g, 3.77 mmol) in acetone (8 mL) was added solid potassium thiocyanate (0.733 g, 7.55 mmol). The reaction mixture was heated to reflux for 2.5 h. Cesium carbonate (0.246 g, 0.76 mmol) was added, and the reaction mixture was stirred at room temperature. The reaction mixture was filtered through diatomaceous earth, and the cake was washed with acetone. The solvent was concentrated and the residue was loaded onto diatomaceous earth. Purification by flash chromatography eluting with a gradient of EtOAc-hexanes provided the title compound as a pink solid (644 mg, 56%): mp 128-131° C.; ¹H NMR (400 MHz, CDCl₃) δ 7.90 (s, 1H), 7.72 (d, J=8.8 Hz, 1H), 7.12-6.99 (m, 1H), 6.81 (d, J=2.5 Hz, 1H), 4.24-3.99 (m, 2H), 3.87 (s, 3H); ¹⁹F NMR (376 MHz, CDCl₃) δ −60.59; EIMS m/z 290; HRMS-ESI (m/z) [M+H]⁺ calcd for C₁₁H₉F₃N₂O₂S, 291.0410; found, 291.0410.

The following compounds were prepared in accordance to the procedure in Example 38.

### 3-(5-Ethoxy-2-(trifluoromethyl)phenyl)-2-iminothiazolidin-4-one (C141)

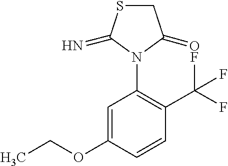 [SMILES: CCOc1ccc(C(F)(F)F)c(N2C(=N)SCC2=O)c1]

The title compound was prepared from 2-chloro-N-(5-ethoxy-2-(trifluoromethyl)phenyl)acetamide (C202) and was isolated as an orange solid (623 mg, 65%): mp 73-78° C.; ¹H NMR (400 MHz, CDCl₃) δ 7.90 (s, 1H), 7.71 (d, J=8.9 Hz, 1H), 7.05 (d, J=8.9 Hz, 1H), 6.79 (d, J=2.4 Hz, 1H), 4.21-3.85 (m, 4H), 1.43 (t, J=7.0 Hz, 3H); ¹⁹F NMR (376 MHz, CDCl₃) δ −60.57; HRMS-ESI (m/z) [M+H]+ calcd for C₁₂H₁₁F₃N₂O₂S, 305.0566; found, 305.0562.

### 2-Imino-3-(5-methoxy-2-(2,2,2-trifluoroethoxy)phenyl)thiazolidin-4-one (C142)

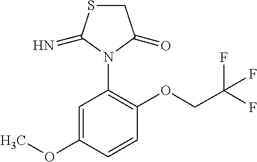 [SMILES: COc1ccc(OCC(F)(F)F)c(N2C(=N)SCC2=O)c1]

The title compound was prepared from 2-chloro-N-(5-methoxy-2-(2,2,2-trifluoroethoxy)phenyl)acetamide (C203) and was isolated as an orange oil (576 mg, 59%): ¹H NMR (400 MHz, CDCl₃) δ 7.83 (s, 1H), 7.08-6.93 (m, 2H), 6.80 (s, 1H), 4.30 (qd, J=8.1, 2.2 Hz, 2H), 4.12-3.93 (m, 2H), 3.79 (s, 3H); ¹⁹F NMR (376 MHz, CDCl₃) δ −74.39; HRMS-ESI (m/z) [M+H]⁺ calcd for C₁₂H₁₁F₃N₂O₃S, 321.0515; found, 321.0523.

### 3-(4-Fluoro-2-isopropyl-5-methoxyphenyl)-2-iminothiazolidin-4-one (C143)

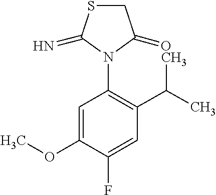 [SMILES: COc1cc(N2C(=N)SCC2=O)c(C(C)C)cc1F]

The title compound was prepared from 2-chloro-N-(4-fluoro-2-isopropyl-5-methoxyphenyl)acetamide (C204) and was isolated as a tan solid (474 mg, 61%): mp 149-151° C.; ¹H NMR (400 MHz, CDCl₃) δ 7.88 (s, 1H), 7.13 (d, J=12.4 Hz, 1H), 6.67 (d, J=7.9 Hz, 1H), 4.12 (d, J=2.8 Hz, 2H), 3.86 (d, J=3.4 Hz, 3H), 2.70-2.51 (m, 1H), 1.17 (t, J=7.1 Hz, 6H); ¹⁹F NMR (376 MHz, CDCl₃) δ −132.04; HRMS-ESI (m/z) [M+H]⁺ calcd for C₁₃H₁₅FN₂O₂S, 283.0911; found, 283.0913.

### 2-Imino-3-(2-(1-methoxyethyl)-4-methylphenyl)thiazolidin-4-one (C144)

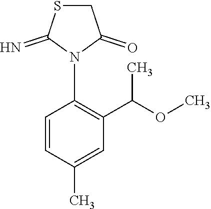 [SMILES: COC(C)c1cc(C)ccc1N1C(=N)SCC1=O]

The title compound was prepared from 2-chloro-N-(2-(1-methoxyethyl)-4-methylphenyl)acetamide (C205) and was isolated as a yellow waxy solid (381 mg, 57%): ¹H NMR (400 MHz, CDCl₃) δ 7.87 (d, J=14.1 Hz, 1H), 7.41 (s, 1H), 7.23 (d, J=8.2 Hz, 1H), 7.00 (d, J=8.0 Hz, 1H), 4.23-3.94 (m, 3H), 3.14 (d, J=1.5 Hz, 3H), 2.43 (d, J=14.0 Hz, 3H), 1.38 (dd, J=9.9, 6.4 Hz, 3H); HRMS-ESI (m/z) [M+H]+ calcd for C₁₃H₁₆N₂O₂S, 265.1005; found, 265.1004.

### 2-Imino-3-(5-methoxy-2-(1-methoxyethyl)phenyl)thiazolidin-4-one (C145)

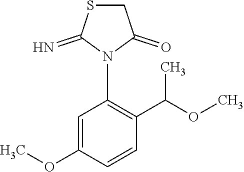

The title compound was prepared from 2-chloro-N-(5-methoxy-2-(1-methoxyethyl)phenyl)acetamide (C206) and was isolated as a yellow oil (41 mg, 51%): ¹H NMR (400 MHz, CDCl₃) δ 7.90 (d, J=15.0 Hz, 1H), 7.51 (d, J=6.4 Hz, 1H), 7.07 (s, 1H), 6.63 (s, 1H), 4.12 (p, J=6.4, 5.7 Hz, 3H), 3.81 (s, 3H), 3.11 (d, J=2.3 Hz, 3H), 1.49-1.31 (m, 3H); EIMS m/z 282.

### 3-(2-(sec-Butoxy)-5-methylphenyl)-2-iminothiazolidin-4-one (C146)

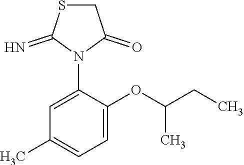 [SMILES: CCC(C)Oc1ccc(C)cc1N1C(=N)SCC1=O]

The title compound was prepared from N-(2-(sec-butoxy)-5-methylphenyl)-2-chloroacetamide (C195) and was isolated as a yellow oil (528 mg, 75%): ¹H NMR (400 MHz, CDCl₃) δ 8.42 (s, 1H), 8.19-8.11 (m, 1H), 6.88 (ddd, J=8.4, 2.2, 0.8 Hz, 1H), 6.79 (d, J=8.3 Hz, 1H), 4.36 (h, J=6.1 Hz, 1H), 3.90 (s, 2H), 2.30 (d, J=0.7 Hz, 3H), 1.80 (ddd, J=13.7, 7.6, 6.2 Hz, 1H), 1.74-1.61 (m, 1H), 1.33 (d, J=6.1 Hz, 3H), 1.00 (t, J=7.5 Hz, 3H); ¹³C NMR (126 MHz, CDCl₃) δ 161.96, 144.31, 130.52, 127.15, 125.11, 120.47, 112.53, 110.74, 38.02, 29.15, 20.94, 19.34, 9.84.

### 3-(5-Ethoxy-2-isopropylphenyl)-2-iminothiazolidin-4-one (C147)

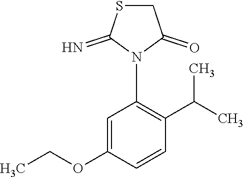 [SMILES: CCOc1ccc(C(C)C)c(N2C(=N)SCC2=O)c1]

The title compound was prepared from 2-chloro-N-(5-ethoxy-2-isopropylphenyl)acetamide (C198) and was isolated as a red oil (350 mg, 77%): ¹H NMR (400 MHz, CDCl₃) δ 7.86 (s, 1H), 7.34 (d, J=10.4 Hz, 1H), 7.00 (s, 1H), 6.59 (s, 1H), 4.14-3.94 (m, 4H), 2.63 (s, 1H), 1.39 (t, J=6.9 Hz, 3H), 1.16 (d, J=6.9 Hz, 6H); HRMS-ESI (m/z) [M+H]⁺ calcd for C₁₄H₁₈N₂O₂S, 279.1162; found, 279.1165.

### 3-(2-Ethyl-5-methoxyphenyl)-2-iminothiazolidin-4-one (C148)

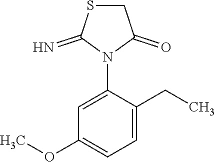 [SMILES: CCc1ccc(OC)cc1N1C(=N)SCC1=O]

The title compound was prepared from 2-chloro-N-(2-ethyl-5-methoxyphenyl)acetamide (C199) and was isolated as a yellow oil (87 mg, 31%): ¹H NMR (400 MHz, CDCl₃) δ 7.86 (s, 1H), 7.31 (s, 1H), 7.00 (s, 1H), 6.64 (s, 1H), 4.12 (m, 2H), 3.79 (s, 3H), 2.41 (s, 2H), 1.16 (t, J=7.6 Hz, 3H); ¹³C NMR (151 MHz, DMSO-d₆) δ 171.03, 157.16, 157.11, 134.03, 132.95, 128.83, 114.26, 113.96, 54.71, 33.14, 22.18, 13.78; EIMS m/z 250.

### Example 39: Preparation of 2-imino-3-(2-(1-(methylthio)ethyl)phenyl)thiazolidin-4-one (C149)

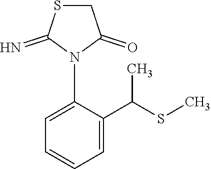 [SMILES: CSC(C)c1ccccc1N1C(=N)SCC1=O]

To a magnetically stirred solution of 2-chloro-N-(2-(1-(methylthio)ethyl)phenyl)acetamide (C197; 3.906 g, 16.02 mmol) in dry acetonitrile (32 mL) was added potassium thiocyanate (3.89 g, 40.1 mmol) in a dry 20 mL vial under a nitrogen atmosphere. The reaction mixture was stirred at 60° C. for 4 h, then cooled to 23° C. Cesium carbonate (0.26 g, 0.80 mmol) was added, and the reaction mixture was allowed to stir for 3 h. The reaction mixture was filtered and loaded onto diatomaceous earth. Purification by flash column chromatography eluting with a hexanes-EtOAc gradient provided the title compound as a tan solid (2.03 g, 43%): mp 101-104° C.; ¹H NMR (400 MHz, CDCl₃) δ 7.79 (ddd, J=18.3, 7.9, 1.5 Hz, 1H), 7.52 (tt, J=7.8, 1.5 Hz, 1H), 7.44-7.33 (m, 1H), 7.10 (dt, J=7.8, 1.5 Hz, 1H), 4.16-4.04 (m, 2H), 3.78 (dq, J=18.8, 7.0 Hz, 1H), 1.82 (d, J=7.3 Hz, 3H), 1.51 (d, J=6.8 Hz, 3H); ¹³C NMR (101 MHz, CDCl₃) δ 171.11, 170.90, 130.65, 130.55, 128.76, 128.70, 128.67, 128.54, 128.40, 128.33, 128.27, 40.32, 39.71, 34.20, 33.92, 22.57, 22.17, 14.83, 14.23.

### Example 40: Preparation of 2-imino-3-(2-isopropyl-5-(methylamino)phenyl)thiazolidin-4-one hydrochloride (C150)

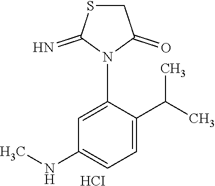

To a 0° C. solution of tert-butyl (3-(2-imino-4-oxothiazolidin-3-yl)-4-isopropylphenyl) (methyl)carbamate (C132; 4.5 g, 11.01 mmol) in 1,4-dioxane (60 mL) was added 4 M HCl in 1,4-dioxane (60 mL), and the reaction mixture was stirred at room temperature for 6 h. The reaction mixture was concentrated under reduced pressure. The title compound was isolated as a cream solid (3.2 g; 92%): ¹H NMR (400 MHz, DMSO-d₆) δ 15.06-10.81 (m, 3H), 7.33 (d, J=8.4 Hz, 1H), 6.90 (d, J=6.8 Hz, 1H), 6.59 (s, 1H), 4.62-4.57 (m, 1H), 4.35-4.30 (m, 1H), 2.69 (s, 3H), 2.55-2.53 (m, 1H), 1.10-1.04 (m, 6H); ESIMS m/z 264 ([M+H]⁺).

The following compounds were prepared in accordance to the procedure in Example 40.

### 3-(5-(Ethylamino)-2-isopropylphenyl)-2-iminothiazolidin-4-one hydrochloride (C151)

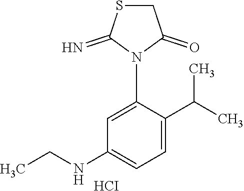

The title compound was prepared from tert-butyl ethyl(3-(2-imino-4-oxothiazolidin-3-yl)-4-isopropylphenyl)carbamate (C133) and was isolated as an off-white solid (3.1 g, 83%): ¹H NMR (400 MHz, DMSO-d₆) δ 12.48-10.71 (m, 3H), 7.45 (d, J=7.8 Hz, 1H), 7.25-7.06 (m, 1H), 6.98-6.75 (m, 1H), 4.63-4.57 (m, 1H), 4.34-4.30 (m, 1H), 3.10 (q, J=6.8 Hz, 2H), 2.63-2.53 (m, 1H), 1.20 (t, J=7.2 Hz, 3H), 1.12-1.05 (m, 6H); ESIMS m/z 278 ([M+H]⁺).

### 2-Imino-3-(2-isopropyl-5-(propylamino)phenyl)thiazolidin-4-one hydrochloride (C152)

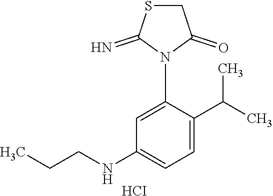

The title compound was prepared from tert-butyl (3-(2-imino-4-oxothiazolidin-3-yl)-4-isopropylphenyl)(propyl)carbamate (C134) and was isolated as an off-white solid (3.2 g, 85%): ¹H NMR (400 MHz, DMSO-d₆) δ 12.46-10.20 (m, 3H), 7.39 (d, J=8.4 Hz, 1H), 7.08 (s, 1H), 6.78-6.76 (m, 1H), 4.62-4.57 (m, 1H), 4.34-4.29 (m, 1H), 3.00 (t, J=7.2 Hz, 2H), 2.59-2.53 (m, 1H), 1.65-1.55 (m, 2H), 1.10 (d, J=6.8 Hz, 3H), 1.05 (d, J=6.8 Hz, 3H), 0.93 (t, J=7.3 Hz, 3H); ESIMS m/z 292 ([M+H]⁺).

### Example 41: Preparation of 2-chloro-N-(5-cyano-2-isopropylphenyl)acetamide (C154)

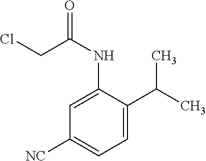 [SMILES: CC(C)c1ccc(C#N)cc1NC(=O)CCl]

To a solution of 3-amino-4-isopropylbenzonitrile (C208; 6.0 g, 37.2 mmol) in EtOAc (300 mL) was added sodium bicarbonate (7.9 g, 93.8 mmol). Chloroacetyl chloride (5.0 mL, 44.68 mmol) was added drop-wise at 0° C., and the reaction mixture was stirred at room temperature for 3 h. The reaction mixture was quenched with water (150 mL) and was extracted with EtOAc (2×100 mL). The organic layer was dried over anhydrous Na₂SO₄, filtered and concentrated under reduced pressure to afford the title compound as a brown solid (4.5 g, 50%): mp 98-101° C.; ¹H NMR (400 MHz, DMSO-d₆) δ 9.93 (s, 1H), 7.77 (d, J=1.6 Hz, 1H), 7.69 (dd, J=1.6, 8.4 Hz, 1H), 7.56 (d, J=8.4 Hz, 1H), 4.34 (s, 2H), 3.28-3.17 (m, 1H), 1.16 (d, J=6.8 Hz, 6H); ESIMS m/z 237 ([M+H]⁺).

The following compounds were prepared in accordance to the procedure in Example 41.

### 2-Chloro-N-(2-isopropyl-5-methoxyphenyl)acetamide (C155)

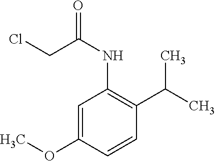 [SMILES: COc1ccc(C(C)C)c(NC(=O)CCl)c1]

The title compound was prepared from 2-isopropyl-5-methoxyaniline (C237) and was isolated as an off-white solid (4.01 g, 90%): ¹H NMR (400 MHz, CDCl₃) δ 8.35 (s, 1H), 7.51 (d, J=2.7 Hz, 1H), 7.20 (d, J=8.6 Hz, 1H), 6.77 (dd, J=8.7, 2.7 Hz, 1H), 4.25 (s, 2H), 3.80 (s, 3H), 2.96 (hept, J=6.8 Hz, 1H), 1.26 (d, J=6.8 Hz, 6H); ESIMS m/z 242 ([M+H]⁺).

### 2-Chloro-N-(5-methyl-2-(3,3,3-trifluoropropoxy)phenyl)acetamide (C156)

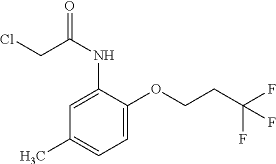 [SMILES: Cc1ccc(OCCC(F)(F)F)c(NC(=O)CCl)c1]

The title compound was prepared from 5-methyl-2-(3,3,3-trifluoropropoxy)aniline (C238) and was isolated as an off-white solid (1.91 g, 90%): ¹H NMR (300 MHz, CDCl₃) δ 8.89 (s, 1H), 8.23-8.15 (m, 1H), 6.89 (ddd, J=8.3, 2.1, 0.8 Hz, 1H), 6.78 (d, J=8.3 Hz, 1H), 4.26 (t, J=6.1 Hz, 2H), 4.19 (s, 2H), 2.66 (qt, J=10.4, 6.1 Hz, 2H), 2.32 (d, J=0.7 Hz, 3H); ESIMS m/z 296 ([M+H]⁺).

### N-(2-(sec-Butyl)-5-methoxyphenyl)-2-chloroacetamide (C157)

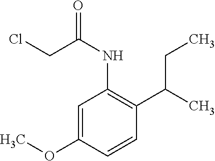 [SMILES: CCC(C)c1ccc(OC)cc1NC(=O)CCl]

The title compound was prepared from 2-(sec-butyl)-5-methoxyaniline (C209) and was isolated as a brown oil that was used without further manipulation (2.0 g): ¹H NMR (400 MHz, CDCl₃) δ 8.36 (s, 1H), 7.46 (d, J=2.8 Hz, 1H), 7.15 (d, J=8.8 Hz, 1H), 6.77 (dd, J=3.2, 5.6 Hz, 1H), 4.26 (s, 2H), 4.13 (s, 1H), 3.79 (s, 3H), 2.72-2.65 (m, 1H), 1.65-1.55 (m, 1H), 1.25 (dd, J=2.4, 6.8 Hz, 3H), 0.87 (t, J=7.2 Hz, 3H); ESIMS m/z 256 ([M+H]⁺).

### 2-Chloro-N-(5-(dimethylamino)-2-isopropylphenyl)acetamide (C158)

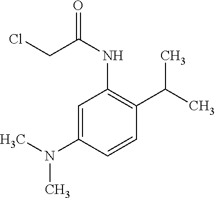 [SMILES: CC(C)c1ccc(N(C)C)cc1NC(=O)CCl]

The title compound was prepared from 4-isopropyl-N,N-dimethylbenzene-1,3-diamine (C207) and was isolated as an off-white solid which was used in the next step without purification: ¹H NMR (300 MHz, DMSO-d₆) δ 9.48 (s, 1H), 7.11 (d, J=6.0 Hz, 1H), 6.65-6.59 (m, 2H), 4.26 (s, 2H), 3.01-2.96 (m, 1H), 2.83 (s, 6H), 1.09 (d, J=6.0 Hz, 6H); ESIMS m/z 255 ([M+H]⁺).

### 2-Chloro-N-(5-chloro-2-(trifluoromethyl)phenyl)acetamide (C159)

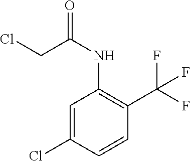 [SMILES: O=C(CCl)Nc1cc(Cl)ccc1C(F)(F)F]

The title compound was prepared from 5-chloro-2-(trifluoromethyl)aniline and was isolated as a white solid (5.3 g, 85%): mp 94-96° C.; ¹H NMR (400 MHz, CDCl₃) δ 8.79 (s, 1H), 8.40-8.35 (m, 1H), 7.58 (d, J=8.5 Hz, 1H), 7.26 (ddt, J=8.5, 1.8, 0.9 Hz, 1H), 4.23 (s, 2H); ¹⁹F NMR (376 MHz, CDCl₃) δ −60.59; ESIMS m/z 272 ([M+H]⁺).

### 2-Chloro-N-(5-methoxy-2-propylphenyl)acetamide (C160)

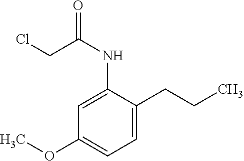 [SMILES: CCCc1ccc(OC)cc1NC(=O)CCl]

The title compound was prepared from 5-methoxy-2-propylaniline (C210) and was isolated as an off-white solid (7.0 g, 82%): ¹H NMR (300 MHz, CDCl₃) δ 8.34 (br s, 1H), 7.65 (d, J=2.4 Hz, 1H), 7.09 (d, J=8.7 Hz, 1H), 6.70 (dd, J=2.7, 8.4 Hz, 1H), 4.23 (s, 2H), 3.78 (s, 3H), 2.53 (t, J=7.5 Hz, 2H), 1.65-1.57 (m, 2H), 0.98 (t, J=7.2 Hz, 3H); ESIMS m/z 242 ([M+H]⁺).

### N-(2-Butyl-5-methylphenyl)-2-chloroacetamide (C161)

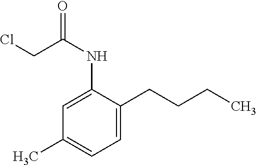 [SMILES: CCCCc1ccc(C)cc1NC(=O)CCl]

The title compound was prepared from 2-butyl-5-methylaniline (C234) and was isolated as an off-white solid that was used without further manipulation (1.5 g): ESIMS m/z 240 ([M+H]⁺).

### N-(2-Butyl-5-methoxyphenyl)-2-chloroacetamide (C162)

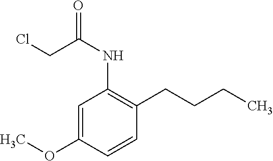 [SMILES: CCCCc1ccc(OC)cc1NC(=O)CCl]

The title compound was prepared from 2-butyl-5-methoxyaniline (C233) and was isolated as an off-white solid that was used without further manipulation (4.6 g): ¹H NMR (300 MHz, CDCl₃) δ 8.34 (br s, 1H), 7.64 (d, J=2.1 Hz, 1H), 7.08 (d, J=8.4 Hz, 1H), 6.70 (dd, J=2.7, 8.7 Hz, 1H), 4.24 (s, 2H), 3.80 (s, 3H), 2.55 (t, J=7.8 Hz, 2H), 1.61-1.51 (m, 2H), 1.45-1.38 (m, 2H), 0.95 (t, J=7.2 Hz, 3H); ESIMS m/z 256 ([M+H]⁺).

### 2-Chloro-N-(3-(dimethylamino)phenyl)acetamide (C163)

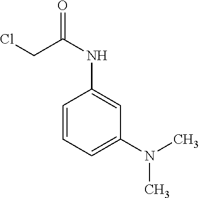

The title compound was prepared from N,N-dimethylbenzene-1,3-diamine and was isolated as a flaky solid (5.5 g, 100%): ¹H NMR (400 MHz, CDCl₃) δ 8.16 (s, 1H), 7.19 (t, J=8.1 Hz, 1H), 7.02 (t, J=2.3 Hz, 1H), 6.80 (ddd, J=7.9, 2.0, 0.8 Hz, 1H), 6.54 (ddd, J=8.4, 2.6, 0.8 Hz, 1H), 4.17 (s, 2H), 2.96 (s, 6H); ESIMS m/z 213 ([M+H]⁺).

### 2-Chloro-N-(5-(dimethylamino)-2-(3,3,3-trifluoropropoxy)phenyl)acetamide (C164)

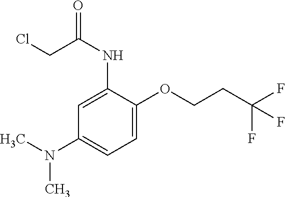 [SMILES: CN(C)c1ccc(OCCC(F)(F)F)c(NC(=O)CCl)c1]

The title compound was prepared from N,N-dimethyl-4-(3,3,3-trifluoropropoxy)benzene-1,3-diamine (C241) and was isolated as a light brown oil (2.15 g, 98%): ¹H NMR (400 MHz, CDCl₃) δ 8.89 (s, 1H), 7.95 (d, J=3.0 Hz, 1H), 6.81 (d, J=9.0 Hz, 1H), 6.46 (dd, J=8.9, 3.0 Hz, 1H), 4.22 (t, J=6.1 Hz, 2H), 4.18 (s, 2H), 2.91 (s, 6H), 2.72-2.54 (m, 2H); ¹⁹F NMR (376 MHz, CDCl₃) δ −64.43; ESIMS m/z 325 ([M−H]⁺).

### 2-Chloro-N-(4-(dimethylamino)-2-isopropylphenyl)acetamide (C165)

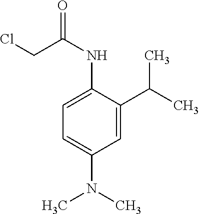 [SMILES: CC(C)c1cc(N(C)C)ccc1NC(=O)CCl]

The title compound was prepared from 3-isopropyl-N,N-dimethylbenzene-1,4-diamine (C239) and was isolated as an off-white solid (1.33 g, 88%): ¹H NMR (400 MHz, CDCl₃) δ 8.02 (s, 1H), 7.39 (d, J=8.7 Hz, 1H), 6.68-6.57 (m, 2H), 4.23 (s, 2H), 3.06-2.97 (m, 1H), 2.95 (s, 6H), 1.26 (d, J=6.9 Hz, 6H); ESIMS m/z 255 ([M+H]⁺).

### 2-Chloro-N-(5-methyl-2-((2,2,2-trifluoroethoxy)methyl)phenyl)acetamide (C166)

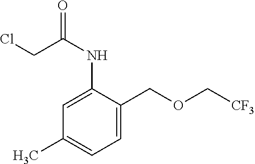 [SMILES: Cc1ccc(COCC(F)(F)F)c(NC(=O)CCl)c1]

The title compound was prepared from 5-methyl-2-((2,2,2-trifluoroethoxy)methyl)aniline (C211) and was isolated as an off-white solid that was used without further manipulation (4.0 g): ESIMS m/z 296 ([M+H]⁺).

### 2-Chloro-N-(5-(diethylamino)-2-isopropylphenyl)acetamide (C167)

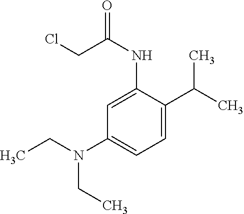 [SMILES: CCN(CC)c1ccc(C(C)C)c(NC(=O)CCl)c1]

The title compound was prepared from N,N-diethyl-4-isopropylbenzene-1,3-diamine (C226) and was isolated as a pale brown solid (4.0 g, 50%): ¹H NMR (400 MHz, DMSO-d₆) δ 9.42 (s, 1H), 7.07 (d, J=9.2 Hz, 1H), 6.56-6.54 (m, 2H), 4.27 (s, 2H), 3.27 (q, J=6.8 Hz, 4H), 2.99-2.95 (m, 1H), 1.12-0.89 (m, 12H); ESIMS m/z 283 ([M+H]⁺).

### 2-Chloro-N-(5-(dipropylamino)-2-isopropylphenyl)acetamide (C168)

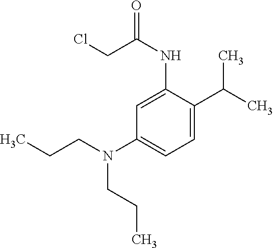 [SMILES: CCCN(CCC)c1ccc(C(C)C)c(NC(=O)CCl)c1]

The title compound was prepared from 4-isopropyl-N,N-dipropylbenzene-1,3-diamine (C227) and was isolated as an off-white solid (2.5 g, 49%): ¹H NMR (400 MHz, DMSO-d₆) δ 9.42 (s, 1H), 7.06 (d, J=8.4 Hz, 1H), 6.53-6.51 (m, 2H), 4.27 (s, 2H), 3.16 (t, J=7.8 Hz, 4H), 2.98-2.94 (m, 1H), 1.54-1.47 (m, 4H), 1.08 (d, J=6.8 Hz, 6H), 0.87 (t, J=7.2 Hz, 6H); ESIMS m/z 311 ([M+H]⁺).

### 2-Chloro-N-(2-(1-ethoxyethyl)-5-methylphenyl)acetamide (C169)

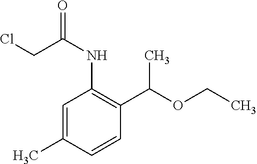 [SMILES: CCOC(C)c1ccc(C)cc1NC(=O)CCl]

The title compound was prepared from 2-(1-ethoxyethyl)-5-methylaniline (C212) and was isolated as an off-white solid that was used without further manipulation (4.5 g): ESIMS m/z 256 ([M+H]⁺).

### 2-Chloro-N-(5-methyl-2-propoxyphenyl)acetamide (C170)

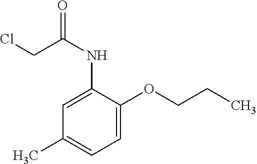 [SMILES: CCCOc1ccc(C)cc1NC(=O)CCl]

The title compound was prepared from 5-methyl-2-propoxyaniline (C213) and was isolated as a pale brown solid that was used without further manipulation (4.0 g): ESIMS m/z 242 ([M+H]⁺).

### 2-Chloro-N-(5-(dimethylamino)-4-fluoro-2-isopropylphenyl)acetamide (C171)

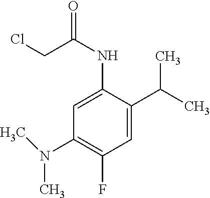 [SMILES: CC(C)c1cc(F)c(N(C)C)cc1NC(=O)CCl]

The title compound was prepared from 6-fluoro-4-isopropyl-N,N-dimethylbenzene-1,3-diamine (C242) and was isolated as a light brown solid (2.18 g, 81%): ¹H NMR (300 MHz, CDCl₃) δ 8.17 (s, 1H), 7.30 (s, 1H), 6.94 (d, J=14.2 Hz, 1H), 4.24 (s, 2H), 2.94 (pd, J=6.9, 1.4 Hz, 1H), 2.83 (d, J=0.8 Hz, 6H), 1.22 (d, J=6.8 Hz, 6H); ESIMS m/z 273 ([M+H]⁺).

### 2-Chloro-N-(2-(ethoxymethyl)-5-methylphenyl)acetamide (C172)

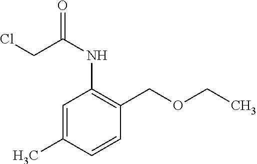 [SMILES: CCOCc1ccc(C)cc1NC(=O)CCl]

The title compound was prepared from 2-(ethoxymethyl)-5-methylaniline (C214) and was isolated as a pale brown solid that was used without further manipulation (4.0 g): ESIMS m/z 242 ([M+H]⁺).

### 2-Chloro-N-(5-methyl-2-(2,2,2-trifluoro-1-methoxyethyl)phenyl)acetamide (C173)

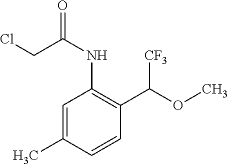 [SMILES: COC(c1ccc(C)cc1NC(=O)CCl)C(F)(F)F]

The title compound was prepared from 5-methyl-2-(2,2,2-trifluoro-1-methoxyethyl)aniline (C236) and was isolated as a brown solid that was used without further manipulation (5.0 g): ¹H NMR (400 MHz, CDCl₃) δ 9.66 (s, 1H), 8.13 (s, 1H), 7.13 (d, J=8.0 Hz, 1H), 7.00 (d, J=8.0 Hz, 1H), 4.17 (s, 2H), 3.54 (s, 3H), 2.38 (s, 3H); ESIMS m/z 296 ([M+H]⁺).

### 2-Chloro-N-(5-(dimethylamino)-2-(trifluoromethyl)phenyl)acetamide (C174)

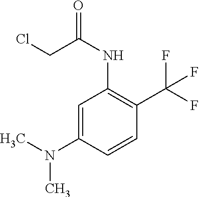 [SMILES: CN(C)c1ccc(C(F)(F)F)c(NC(=O)CCl)c1]

The title compound was prepared from N,N-dimethyl-4-(trifluoromethyl)benzene-1,3-diamine (C223) and was isolated as an off-white solid that was used without further manipulation (5.0 g): ¹H NMR (300 MHz, DMSO-d₆) δ 9.69 (s, 1H), 7.45 (d, J=8.7 Hz, 1H), 6.71-6.66 (m, 2H), 4.28 (s, 2H), 2.96 (s, 6H); ESIMS m/z 281 ([M+H]⁺).

### 2-Chloro-N-(5-(dimethylamino)-2-propylphenyl)acetamide (C175)

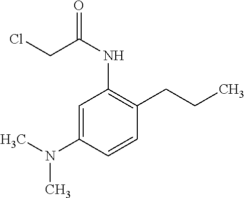 [SMILES: CCCc1ccc(N(C)C)cc1NC(=O)CCl]

The title compound was prepared from N,N-dimethyl-4-propylbenzene-1,3-diamine (C224) and was isolated as a brown solid that was used without further manipulation (5.0 g): ¹H NMR (300 MHz, DMSO-d₆) δ 9.48 (s, 1H), 7.01 (d, J=6.0 Hz, 1H), 6.71 (d, J=3.0 Hz, 1H), 6.56 (dd, J=3.0, 9.0 Hz, 1H), 4.28 (s, 2H), 2.83 (s, 6H), 2.40 (t, J=9.0 Hz, 2H), 1.49-1.41 (m, 2H), 0.85 (t, J=6.0 Hz, 3H); ESIMS m/z 255 ([M+H]⁺).

### 2-Chloro-N-(5-(dimethylamino)-2-(2,2,2-trifluoroethoxy)phenyl)acetamide (C176)

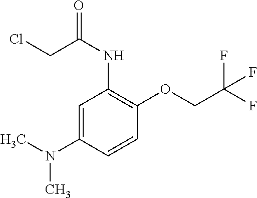 [SMILES: CN(C)c1ccc(OCC(F)(F)F)c(NC(=O)CCl)c1]

The title compound was prepared from N,N-dimethyl-4-(2,2,2-trifluoroethoxy)benzene-1,3-diamine (C225) and was isolated as a brown solid that was used without further manipulation (4.5 g): ¹H NMR (400 MHz, CDCl₃) δ 8.90 (s, 1H), 7.89 (d, J=2.8 Hz, 1H), 6.82 (d, J=8.8 Hz, 1H), 6.43 (dd, J=3.2, 8.8 Hz, 1H), 4.34 (q, J=8.0 Hz, 2H), 4.18 (s, 2H), 2.92 (s, 6H); ESIMS m/z 311 ([M+H]⁺).

### 2-Chloro-N-(5-methyl-2-(4,4,4-trifluorobutoxy)phenyl)acetamide (C177)

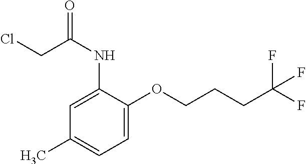 [SMILES: Cc1ccc(OCCCC(F)(F)F)c(NC(=O)CCl)c1]

The title compound was prepared from 5-methyl-2-(4,4,4-trifluorobutoxy)aniline (C240) and was isolated as a flaky solid (4.2 g, 93%): ¹H NMR (400 MHz, CDCl₃) δ 8.96 (s, 1H), 8.17 (d, J=2.0 Hz, 1H), 6.88 (ddd, J=8.3, 2.1, 0.8 Hz, 1H), 6.77 (d, J=8.3 Hz, 1H), 4.20 (s, 2H), 4.08 (t, J=5.9 Hz, 2H), 2.44-2.26 (m, 5H), 2.17-2.06 (m, 2H); ¹⁹F NMR (376 MHz, CDCl₃) δ −66.37; ESIMS m/z 310 ([M−H]⁺).

### 2-Chloro-N-(5-methyl-2-nitrophenyl)acetamide (C178)

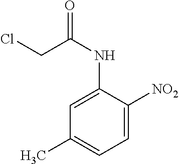

The title compound was prepared from 5-methyl-2-nitroaniline and was isolated as an orange solid (6.8 g, 77%): ¹H NMR (400 MHz, CDCl₃) δ 11.43 (s, 1H), 8.70-8.52 (m, 1H), 8.16 (d, J=8.6 Hz, 1H), 7.05 (ddd, J=8.6, 2.0, 0.9 Hz, 1H), 4.24 (s, 2H), 2.47 (s, 3H); ESIMS m/z 229 ([M+H]⁺). 2-Chloro-N-(2-isobutyl-5-methylphenyl)acetamide (C179)

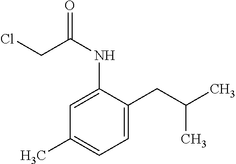 [SMILES: Cc1ccc(CC(C)C)c(NC(=O)CCl)c1]

The title compound was prepared from 2-isobutyl-5-methylaniline (C235) and was isolated as an off-white solid (2.1 g, 72%): ESIMS m/z 240 ([M+H]⁺).
N-(2,5-Bis(trifluoromethyl)phenyl)-2-chloroacetamide (C180)

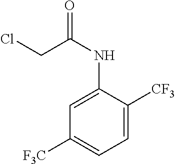 [SMILES: O=C(CCl)Nc1cc(C(F)(F)F)ccc1C(F)(F)F]

The title compound was prepared from 2,5-bis(trifluoromethyl)aniline and was isolated as an off-white solid (6.5 g, 70%): ¹H NMR (300 MHz, CDCl₃) δ 8.88 (br s, 1H), 8.65 (s, 1H), 8.79 (d, J=8.4 Hz, 1H), 7.56-7.53 (m, 1H), 4.26 (s, 2H); ESIMS m/z 304 ([M−H]⁻).

### N-(5-Acetyl-2-isopropylphenyl)-2-chloroacetamide (C181)

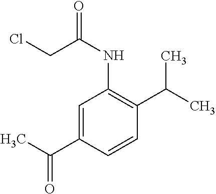 [SMILES: CC(=O)c1ccc(C(C)C)c(NC(=O)CCl)c1]

The title compound was prepared from 1-(3-amino-4-isopropylphenyl)ethan-1-one (C218) and was isolated as an off-white solid (2.5 g, 65%): mp 96-100° C.; ¹H NMR (400 MHz, DMSO-d₆) δ 9.87 (s, 1H), 7.84-7.82 (m, 2H), 7.50 (d, J=8.0 Hz, 1H), 4.33 (s, 2H), 3.22-3.15 (m, 1H), 2.51 (s, 3H), 1.16 (d, J=6.8 Hz, 6H); ESIMS m/z 252 ([M−H]⁻).

### 2-Chloro-N-(5-methoxy-2-(3,3,3-trifluoropropoxy)phenyl)acetamide (C182)

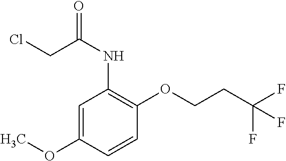 [SMILES: COc1ccc(OCCC(F)(F)F)c(NC(=O)CCl)c1]

The title compound was prepared from 5-methoxy-2-(3,3,3-trifluoropropoxy)aniline (C217) and was isolated as a pale pink solid (4.0 g, 67%): mp 82-84° C.; ¹H NMR (400 MHz, DMSO-d₆) δ 9.23 (s, 1H), 7.69 (d, J=2.4 Hz, 1H), 7.07 (d, J=8.8 Hz, 1H), 6.68 (dd, J=2.8, 8.8 Hz, 1H), 4.38 (s, 2H), 4.21 (t, J=6.0 Hz, 2H), 3.70 (s, 3H), 2.86-2.75 (m, 2H); ¹⁹F NMR (376 MHz, DMSO-d₆) δ −63.04; ESIMS m/z 312 ([M+H]⁺).

### 2-Chloro-N-(2-(1-methoxyethyl)phenyl)acetamide (C183)

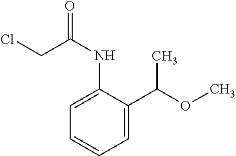

The title compound was prepared from 2-(1-methoxyethyl)aniline (C255) and was isolated as a tan oil (4.71 g, 92%): ¹H NMR (400 MHz, CDCl₃) δ 10.26 (s, 1H), 8.30 (d, J=8.1 Hz, 1H), 7.36-7.28 (m, 1H), 7.16-7.06 (m, 2H), 4.48 (q, J=6.7 Hz, 1H), 4.22 (dd, J=5.2, 0.8 Hz, 2H), 3.34 (s, 3H), 1.53 (d, J=6.7 Hz, 3H); EIMS m/z 228.

### 2-Chloro-N-(5-cyano-2-(3,3,3-trifluoropropoxy)phenyl)acetamide (C184)

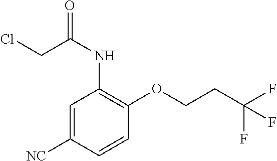 [SMILES: N#Cc1ccc(OCCC(F)(F)F)c(NC(=O)CCl)c1]

The title compound was prepared from 3-amino-4-(3,3,3-trifluoropropoxy)benzonitrile (C215) and was isolated as an off-white solid (1.9 g, 89%): mp 90-94° C.; ¹H NMR (400 MHz, DMSO-d₆) δ 9.49 (s, 1H), 8.36 (d, J=2.0 Hz, 1H), 7.65 (dd, J=2.0, 8.4 Hz, 1H), 7.35 (d, J=8.8 Hz, 1H), 4.46-4.35 (m, 4H), 2.97-2.84 (m, 2H); ¹⁹F NMR (376 MHz, DMSO-d₆) δ−63.16; ESIMS m/z 307 ([M+H]⁺).

### Methyl 3-(2-chloroacetamido)-4-isopropylbenzoate (C185)

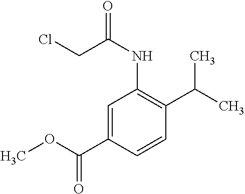 [SMILES: COC(=O)c1ccc(C(C)C)c(NC(=O)CCl)c1]

The title compound was prepared from methyl 3-amino-4-isopropylbenzoate (C216) and was isolated as a pale pink solid (4.0 g, 87%): mp 98-101° C.; ¹H NMR (400 MHz, DMSO-d₆) δ 9.86 (s, 1H), 7.89 (d, J=1.2 Hz, 1H), 7.81 (dd, J=1.2, 8.4 Hz, 1H), 7.50 (d, J=8.4 Hz, 1H), 4.33 (s, 2H), 3.84 (s, 3H), 3.24-3.18 (m, 1H), 1.16 (d, J=6.8 Hz, 6H); ESIMS m/z 268 ([M−H]⁻]).

### 2-Chloro-N-(2,5-diisopropylphenyl)acetamide (C186)

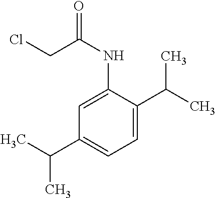 [SMILES: CC(C)c1ccc(C(C)C)c(NC(=O)CCl)c1]

The title compound was prepared from 2,5-diisopropylaniline (C243) and was isolated as a light yellow solid (3.9 g, 85%): ¹H NMR (300 MHz, CDCl₃) δ 8.28 (s, 1H), 7.64 (d, J=1.9 Hz, 1H), 7.23 (d, J=8.1 Hz, 1H), 7.09 (dd, J=8.1, 1.9 Hz, 1H), 4.25 (s, 2H), 2.95 (dp, J=29.8, 6.9 Hz, 2H), 1.26 (dd, J=6.9, 5.8 Hz, 12H); ESIMS m/z 254 ([M+H]⁺).

### 2-Chloro-N-(5-hydroxy-2-isopropylphenyl)acetamide (C187)

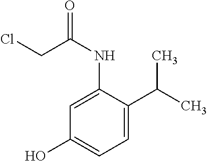

The title compound was prepared from 3-amino-4-isopropylphenol (C244) and was isolated as white crystals (762 mg, 70%): ¹H NMR (400 MHz, CDCl₃) δ 8.49 (s, 1H), 7.71 (d, J=2.7 Hz, 1H), 7.16 (d, J=8.5 Hz, 1H), 6.76 (s, 1H), 6.71 (dd, J=8.5, 2.7 Hz, 1H), 4.28 (s, 2H), 2.95 (hept, J=6.8 Hz, 1H), 1.27 (d, J=6.8 Hz, 6H); ¹³C NMR (101 MHz, CDCl₃) δ 164.42, 154.66, 133.71, 130.10, 126.71, 113.28, 109.36, 43.21, 27.61, 22.96; ESIMS m/z 228 ([M−H]⁺).

### 2-Chloro-N-(2-(piperidin-1-yl)phenyl)acetamide (C188)

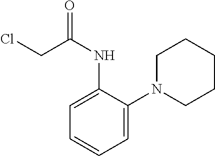 [SMILES: O=C(CCl)Nc1ccccc1N1CCCCC1]

The title compound was prepared from 2-(piperidin-1-yl)aniline and was isolated as a black solid (2.76 g, 75%): ¹H NMR (400 MHz, CDCl₃) δ 9.90 (s, 1H), 8.37 (dd, J=7.9, 1.8 Hz, 1H), 7.22-7.02 (m, 3H), 4.23 (s, 2H), 2.81 (t, J=5.3 Hz, 4H), 1.77 (p, J=5.7 Hz, 4H), 1.60 (s, 2H); ESIMS m/z 253 ([M+H]⁺).

### tert-Butyl (3-(2-chloroacetamido)-4-isopropylphenyl)(methyl)carbamate (C189)

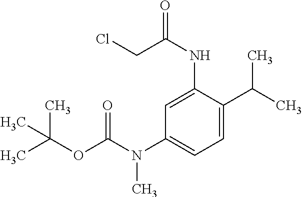 [SMILES: CC(C)c1ccc(N(C)C(=O)OC(C)(C)C)cc1NC(=O)CCl]

The title compound was prepared from tert-butyl (3-amino-4-isopropylphenyl)(methyl)carbamate (C219) and was isolated as an off-white solid (5.0 g, 67%): ¹H NMR (400 MHz, DMSO-d₆) δ 9.70 (s, 1H), 7.28 (d, J=8.8 Hz, 1H), 7.20 (d, J=2.4 Hz, 1H), 7.14 (dd, J=2.0, 8.4 Hz, 1H), 4.29 (s, 2H), 3.15 (s, 3H), 3.13-3.08 (m, 1H), 1.40 (s, 9H), 1.14 (d, J=6.8 Hz, 6H); ESIMS m/z 339 ([M−H]⁻).

### tert-Butyl (3-(2-chloroacetamido)-4-isopropylphenyl)(ethyl)carbamate (C190)

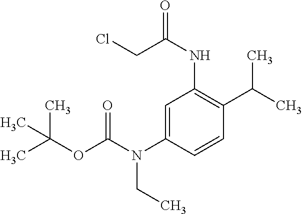 [SMILES: CCN(C(=O)OC(C)(C)C)c1ccc(C(C)C)c(NC(=O)CCl)c1]

The title compound was prepared from tert-butyl (3-amino-4-isopropylphenyl)(ethyl)carbamate (C220) and was isolated as a pale brown solid (6.0 g, 78%): mp 120-122° C.; ¹H NMR (400 MHz, DMSO-d₆) δ 9.69 (s, 1H), 7.29 (d, J=8.0 Hz, 1H), 7.16 (d, J=2.0 Hz, 1H), 7.08 (d, J=8.4 Hz, 1H), 4.30 (s, 2H), 3.56 (d, J=6.8 Hz, 2H), 3.13-3.09 (m, 1H), 1.39 (s, 9H), 1.14 (d, J=6.8 Hz, 6H), 1.05 (t, J=6.8 Hz, 3H); ESIMS m/z 355 ([M+H]⁺).

### tert-Butyl (3-(2-chloroacetamido)-4-isopropylphenyl)(propyl)carbamate (C191)

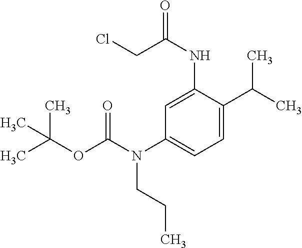 [SMILES: CCCN(C(=O)OC(C)(C)C)c1ccc(C(C)C)c(NC(=O)CCl)c1]

The title compound was prepared from tert-butyl (3-amino-4-isopropylphenyl)(propyl)carbamate (C221) and was isolated as an off-white solid (11.0 g, 87%): ¹H NMR (400 MHz, DMSO-d₆) δ 9.72 (s, 1H), 7.29 (d, J=8.8 Hz, 1H), 7.17 (d, J=2.0 Hz, 1H), 7.08 (dd, J=2.0, 8.4 Hz, 1H), 4.30 (s, 2H), 3.50 (t, J=7.2 Hz, 2H), 3.13-3.09 (m, 1H), 1.47-1.41 (m, 2H), 1.38 (s, 9H), 1.14 (d, J=6.8 Hz, 6H), 0.82 (t, J=7.2 Hz, 3H); ESIMS m/z 369 ([M+H]⁺).

### 2-Chloro-N-(2-isopropyl-5-(N-methylacetamido)phenyl)acetamide (C193)

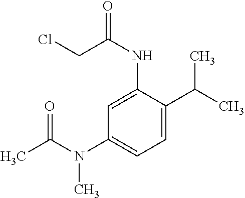 [SMILES: CC(=O)N(C)c1ccc(C(C)C)c(NC(=O)CCl)c1]

The title compound was prepared from N-(3-amino-4-isopropylphenyl)-N-methylacetamide (C228) and was isolated as a white powder (1.91 g, 95%): ¹H NMR (400 MHz, CDCl₃) δ 8.45 (s, 1H), 7.83 (d, J=2.2 Hz, 1H), 7.33 (d, J=8.3 Hz, 1H), 7.01 (dd, J=8.2, 2.3 Hz, 1H), 4.26 (s, 2H), 3.25 (s, 3H), 3.04 (p, J=6.9 Hz, 1H), 1.91 (s, 3H), 1.31 (d, J=6.8 Hz, 6H); ESIMS m/z 283 ([M+H]⁺).

### 2-Chloro-N-(5-(N-ethylacetamido)-2-isopropylphenyl)acetamide (C194)

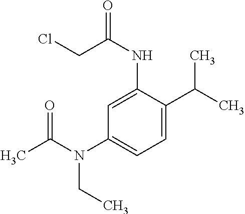 [SMILES: CCN(C(C)=O)c1ccc(C(C)C)c(NC(=O)CCl)c1]

The title compound was prepared from N-(3-amino-4-isopropylphenyl)-N-ethylacetamide (C229) and was isolated as a white powder (1.72 g, 96%): ¹H NMR (400 MHz, CDCl₃) δ 8.45 (s, 1H), 7.80 (d, J=2.2 Hz, 1H), 7.33 (d, J=8.2 Hz, 1H), 6.99 (dd, J=8.2, 2.3 Hz, 1H), 4.26 (s, 2H), 3.74 (q, J=7.1 Hz, 2H), 3.04 (p, J=6.8 Hz, 1H), 1.86 (s, 3H), 1.32 (d, J=6.8 Hz, 6H), 1.12 (t, J=7.1 Hz, 3H); ESIMS m/z 297 ([M+H]⁺).

### N-(2-(sec-Butoxy)-5-methylphenyl)-2-chloroacetamide (C195)

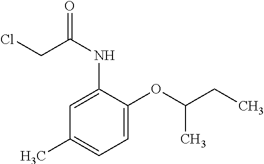 [SMILES: CCC(C)Oc1ccc(C)cc1NC(=O)CCl]

The title compound was prepared from 2-(sec-butoxy)-5-methylaniline (C230) and was isolated as a red-colored semi-solid (4.5 g, 68%): ¹H NMR (300 MHz, CDCl₃) δ 9.10 (br s, 1H), 8.18 (s, 1H), 6.87-6.78 (m, 2H), 4.37-4.30 (m, 1H), 4.16 (s, 2H), 2.31 (s, 3H), 1.83-1.63 (m, 2H), 1.57-1.23 (m, 3H), 1.00 (t, J=7.2 Hz, 3H); ESIMS m/z 256 ([M+H]⁺).

### N-(2-(sec-Butyl)-5-methylphenyl)-2-chloroacetamide (C196)

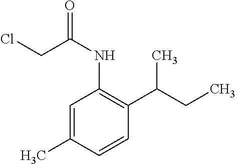 [SMILES: CCC(C)c1ccc(C)cc1NC(=O)CCl]

The title compound was prepared from 2-(sec-butyl)-5-methylaniline (C231) and was isolated as a brown oil that was used without further manipulation (2.8 g); ESIMS m/z 240 ([M+H]⁺).

### 2-Chloro-N-(2-(1-(methylthio)ethyl)phenyl)acetamide (C197)

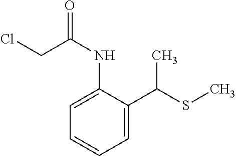

The title compound was prepared from 2-(1-(methylthio)ethyl)aniline (C254) and was isolated as a red wax (3.91 g, 77%): ¹H NMR (400 MHz, CDCl₃) δ 9.46 (s, 1H), 7.87 (dd, J=8.1, 1.3 Hz, 1H), 7.37-7.29 (m, 2H), 7.17 (td, J=7.6, 1.3 Hz, 1H), 4.25 (d, J=2.6 Hz, 2H), 3.98 (q, J=7.1 Hz, 1H), 1.98 (s, 3H), 1.69 (d, J=7.0 Hz, 3H); ¹³C NMR (126 MHz, CDCl₃) δ 164.36, 134.62, 132.81, 127.99, 127.77, 125.52, 124.47, 43.10, 41.58, 19.14, 13.59.

### 2-Chloro-N-(5-ethoxy-2-isopropylphenyl)acetamide (C198)

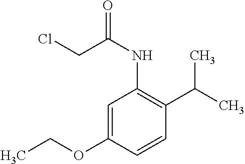 [SMILES: CCOc1ccc(C(C)C)c(NC(=O)CCl)c1]

The title compound was prepared from 5-ethoxy-2-isopropylaniline (C245) and was isolated as a dark teal solid (398 mg, 84%): ¹H NMR (400 MHz, CDCl₃) δ 8.35 (s, 1H), 7.49 (d, J=2.7 Hz, 1H), 7.19 (d, J=8.6 Hz, 1H), 6.76 (dd, J=8.7, 2.7 Hz, 1H), 4.25 (s, 2H), 4.03 (q, J=7.0 Hz, 2H), 2.96 (p, J=6.8 Hz, 1H), 1.40 (t, J=7.0 Hz, 3H), 1.26 (d, J=6.8 Hz, 6H); ¹³C NMR (126 MHz, CDCl₃) δ 163.90, 157.36, 133.93, 131.26, 126.37, 112.83, 108.96, 63.59, 43.25, 27.60, 23.01, 14.80; HRMS-ESI (m/z) [M+H]⁺ calcd for C₁₃H₁₈ClNO₂, 256.1099; found, 256.1094.

### 2-Chloro-N-(2-ethyl-5-methoxyphenyl)acetamide (C199)

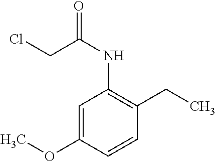

The title compound was prepared from 2-ethyl-5-methoxyaniline (C246) and was isolated as a brown oil that was carried on without further manipulation (242 mg, 99%).

### 2-Chloro-N-(5-ethoxy-2-propylphenyl)acetamide (C200)

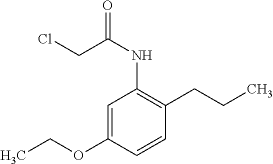 [SMILES: CCCc1ccc(OCC)cc1NC(=O)CCl]

The title compound was prepared from 5-ethoxy-2-propylaniline (C232) and was isolated as a brown solid (1.14 g, 84%): ¹H NMR (300 MHz, CDCl₃) δ 8.35 (s, 1H), 7.62 (d, J=2.7 Hz, 1H), 7.08 (d, J=8.4 Hz, 1H), 6.69 (dd, J=8.5, 2.7 Hz, 1H), 4.25 (s, 2H), 4.03 (q, J=7.0 Hz, 2H), 2.64-2.35 (m, 2H), 1.61 (h, J=7.4 Hz, 2H), 1.40 (t, J=7.0 Hz, 3H), 0.98 (t, J=7.3 Hz, 3H); ¹³C NMR (126 MHz, CDCl₃) δ 163.79, 157.73, 134.89, 130.41, 124.55, 112.22, 108.16, 63.61, 43.26, 32.82, 23.27, 14.80, 13.88; EIMS m/z 255.

### Example 42: Preparation of 2-chloro-N-(5-methoxy-2-(trifluoromethyl)phenyl)acetamide (C201)

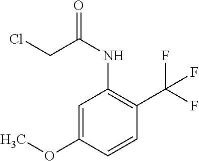 [SMILES: COc1ccc(C(F)(F)F)c(NC(=O)CCl)c1]

To 5-methoxy-2-(trifluoromethyl)aniline (C247; 805 mg, 4.21 mmol) in EtOAc (8.4 mL) in an ice bath were added sodium bicarbonate (708 mg, 8.42 mmol) and 2-chloroacetyl chloride (0.4 mL, 5.05 mmol). The reaction mixture was stirred at room temperature for 90 min, diluted with EtOAc and water, and filtered through a universal phase separator. The organic layer was concentrated under nitrogen. The title compound was isolated as an orange wax (1.02 g, 86%): ¹H NMR (400 MHz, CDCl₃) δ 8.78 (s, 1H), 7.89 (d, J=2.5 Hz, 1H), 7.54 (d, J=8.8 Hz, 1H), 6.84-6.72 (m, 1H), 4.23 (s, 2H), 3.86 (s, 3H); ¹⁹F NMR (376 MHz, CDCl₃) δ −59.33; EIMS m/z 267.

The following compounds were prepared in accordance to the procedure in Example 42.

### 2-Chloro-N-(5-ethoxy-2-(trifluoromethyl)phenyl)acetamide (C202)

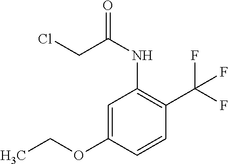 [SMILES: CCOc1ccc(C(F)(F)F)c(NC(=O)CCl)c1]

The title compound was prepared from 5-ethoxy-2-(trifluoromethyl)aniline (C248) and was isolated as a tan solid (855 mg, 83%): ¹H NMR (400 MHz, CDCl₃) δ 8.76 (s, 1H), 7.86 (d, J=2.4 Hz, 1H), 7.53 (d, J=8.8 Hz, 1H), 6.80-6.71 (m, 1H), 4.22 (s, 2H), 4.09 (q, J=7.0 Hz, 2H), 1.43 (t, J=7.0 Hz, 3H); ¹⁹F NMR (376 MHz, CDCl₃) δ −59.29; HRMS-ESI (m/z) [M+H]⁺ calcd for C₁₁H₁₁ClF₃NO₂, 282.0503; found, 282.0501.

### 2-Chloro-N-(5-methoxy-2-(2,2,2-trifluoroethoxy)phenyl)acetamide (C203)

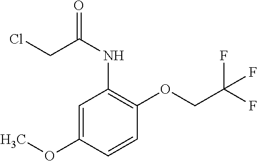 [SMILES: COc1ccc(OCC(F)(F)F)c(NC(=O)CCl)c1]

The title compound was prepared from 5-methoxy-2-(2,2,2-trifluoroethoxy)aniline (C249) and was isolated as an off-white solid (876 mg, 90%): mp 62.5-63.5° C.; ¹H NMR (400 MHz, CDCl₃) δ 8.97 (s, 1H), 8.07 (d, J=3.0 Hz, 1H), 6.84 (d, J=9.0 Hz, 1H), 6.63 (dd, J=9.0, 3.0 Hz, 1H), 4.38 (q, J=8.0 Hz, 2H), 4.20 (s, 2H), 3.80 (s, 3H); ¹⁹F NMR (376 MHz, CDCl₃) δ−74.24; HRMS-ESI (m/z) [M+H]⁺ calcd for C₁₁H₁₁ClF₃NO₃, 298.0452; found, 298.0450.

### 2-Chloro-N-(4-fluoro-2-isopropyl-5-methoxyphenyl)acetamide (C204)

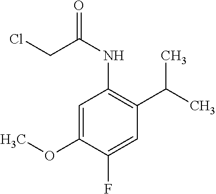 [SMILES: COc1cc(NC(=O)CCl)c(C(C)C)cc1F]

The title compound was prepared from 4-fluoro-2-isopropyl-5-methoxyaniline (C250) and was isolated as a dark green solid (680 mg, 75%): mp 93-96° C.; ¹H NMR (400 MHz, CDCl₃) δ 8.25 (s, 1H), 7.50 (d, J=8.3 Hz, 1H), 7.01 (d, J=12.6 Hz, 1H), 4.25 (s, 2H), 3.88 (s, 3H), 2.97 (m, 1H), 1.24 (d, J=6.8 Hz, 6H); ¹⁹F NMR (376 MHz, CDCl₃) δ −136.86; HRMS-ESI (m/z) [M+H]⁺ calcd for C₁₂H₁₅ClFNO₂, 260.0848; found, 260.0843.

### 2-Chloro-N-(2-(1-methoxyethyl)-4-methylphenyl)acetamide (C205)

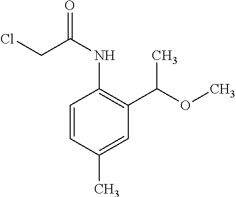 [SMILES: COC(C)c1cc(C)ccc1NC(=O)CCl]

The title compound was prepared from 2-(1-methoxyethyl)-4-methylaniline (C251) and was isolated as a brown oil (554 mg, 90%): ¹H NMR (400 MHz, CDCl₃) δ 10.12 (s, 1H), 8.17 (d, J=8.3 Hz, 1H), 7.12 (dd, J=8.4, 2.0 Hz, 1H), 6.92 (d, J=2.0 Hz, 1H), 4.42 (q, J=6.7 Hz, 1H), 4.20 (d, J=5.6 Hz, 2H), 3.33 (s, 3H), 2.31 (s, 3H), 1.51 (d, J=6.7 Hz, 3H); ¹³C NMR (126 MHz, CDCl₃) δ 170.35, 164.35, 134.24, 133.01, 130.37, 128.97, 121.69, 81.20, 56.40, 43.06, 40.51, 20.94; HRMS-ESI (m/z) [M+Na]⁺ calcd for C₁₂H₁₆ClNO₂, 264.0762; found, 264.0760.

### 2-Chloro-N-(5-methoxy-2-(1-methoxyethyl)phenyl)acetamide (C206)

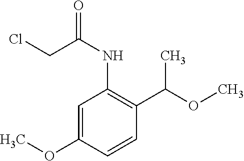 [SMILES: COc1ccc(C(C)OC)c(NC(=O)CCl)c1]

The title compound was prepared from 5-methoxy-2-(1-methoxyethyl)aniline (C252) and was isolated as a yellow oil (51 mg, 58%): ¹H NMR (400 MHz, CDCl₃) δ 10.24 (s, 1H), 8.02 (d, J=2.6 Hz, 1H), 7.01 (d, J=8.4 Hz, 1H), 6.63 (dd, J=8.4, 2.6 Hz, 1H), 4.43 (q, J=6.7 Hz, 1H), 4.19 (d, J=3.2 Hz, 2H), 3.82 (s, 3H), 3.32 (s, 3H), 1.51 (d, J=6.7 Hz, 3H); EIMS m/z 257.

### Example 43: Preparation of 4-isopropyl-N,N-dimethylbenzene-1,3-diamine (C207)

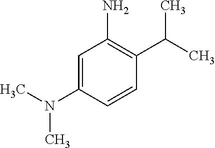

N,N-Dimethyl-3-nitro-4-(prop-1-en-2-yl)aniline (C266; 1.0 g, 4.85 mmol) was dissolved in ethanol (9.70 mL) and EtOAc (9.70 mL) and 10% palladium on carbon (0.52 g, 0.24 mmol) was added. The flask was evacuated and refilled with an atmosphere of hydrogen. The reaction mixture was stirred at room temperature overnight and was filtered through a pad of diatomaceous earth. The filtrate was concentrated under reduced pressure. Purification by silica gel column chromatography eluting with 0-40% EtOAc-hexanes afforded the title compound as a pale yellow non-viscous oil (610 mg, 71%): ¹H NMR (400 MHz, CDCl₃) δ 7.01 (d, J=8.5 Hz, 1H), 6.25 (dd, J=8.5, 2.6 Hz, 1H), 6.11 (d, J=2.6 Hz, 1H), 3.60 (s, 2H), 2.88 (s, 6H), 2.82 (q, J=6.8 Hz, 1H), 1.23 (d, J=6.8 Hz, 6H); ¹³C NMR (126 MHz, CDCl₃) δ 149.83, 143.92, 126.03, 121.91, 104.39, 100.64, 40.81, 27.02, 22.63; ESIMS m/z 178 ([M+H]⁺)

The following compounds were prepared in accordance to the procedure in Example 43.

### 3-Amino-4-isopropylbenzonitrile (C208)

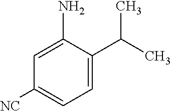

The title compound was prepared from 3-nitro-4-(prop-1-en-2-yl)benzonitrile (C263) and was isolated as a brown solid (6.5 g, 75%): mp 46-50° C.; ¹H NMR (400 MHz, DMSO-d₆) δ 7.15 (d, J=8.4 Hz, 1H), 6.98-6.84 (m, 2H), 5.41 (s, 2H), 3.02-2.96 (m, 1H), 1.13 (d, J=6.4 Hz, 6H); ESIMS m/z 161 ([M+H]⁺).

### 2-(sec-Butyl)-5-methoxyaniline (C209)

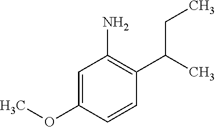

The title compound was prepared from (E)-1-(but-2-en-2-yl)-4-methoxy-2-nitrobenzene (C262) using 40 psi of hydrogen and was isolated as a brown liquid that was used without further manipulation (3 g).

### 5-Methoxy-2-propylaniline (C210)

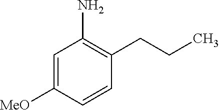

The title compound was prepared from (E)-4-methoxy-2-nitro-1-(prop-1-en-1-yl)benzene and was isolated as a brown oil (5.2 g, 90%): ¹H NMR (300 MHz, CDCl₃) δ 6.93 (d, J=8.4 Hz, 1H), 6.31-6.24 (m, 2H), 3.74 (s, 3H), 3.60 (br s, 2H), 2.40 (t, J=7.8 Hz, 2H), 1.64-1.54 (m, 2H), 0.98 (t, J=7.5 Hz, 3H); ESIMS m/z 166 ([M+H]⁺).

### 5-Methyl-2-((2,2,2-trifluoroethoxy)methyl)aniline (C211)

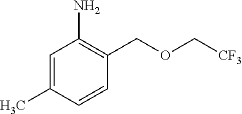

The title compound was prepared from 4-methyl-2-nitro-1-((2,2,2-trifluoroethoxy)methyl)benzene (C307) and was isolated as a brown liquid that was used without further manipulation (3.0 g): ESIMS m/z 220 ([M+H]⁺).

### 2-(1-Ethoxyethyl)-5-methylaniline (C212)

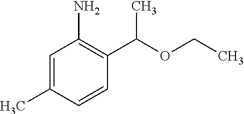

The title compound was prepared from 1-(1-ethoxyethyl)-4-methyl-2-nitrobenzene (C296) and was isolated as a brown liquid that was used without further manipulation (2.8 g): ESIMS m/z 179 ([M]⁺).

### 5-Methyl-2-propoxyaniline (C213)

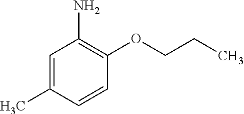

The title compound was prepared from 4-methyl-2-nitro-1-propoxybenzene (C304) and was isolated as a yellow liquid that was used without further manipulation (3.4 g): ESIMS m/z 166 ([M+H]⁺).

### 2-(Ethoxymethyl)-5-methylaniline (C214)

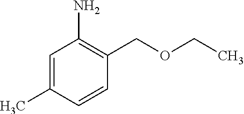

The title compound was prepared from 1-(ethoxymethyl)-4-methyl-2-nitrobenzene (C306) and was isolated as a brown liquid that was used without further manipulation (3.5 g): ESIMS m/z 166 ([M+H]⁺).

### 3-Amino-4-(3,3,3-trifluoropropoxy)benzonitrile (C215)

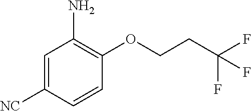 [SMILES: N#Cc1ccc(OCCC(F)(F)F)c(N)c1]

The title compound was prepared from 3-nitro-4-(3,3,3-trifluoropropoxy)benzonitrile (C303) and was isolated as a pale yellow solid (1.6 g, 72%): ¹H NMR (400 MHz, DMSO-d₆) δ 7.02-6.96 (m, 2H), 6.93 (d, J=0.8 Hz, 1H), 5.17 (s, 2H), 4.26 (t, J=6.0 Hz, 2H), 2.91-2.79 (m, 2H); ESIMS m/z 231 ([M+H]⁺).

### Methyl 3-amino-4-isopropylbenzoate (C216)

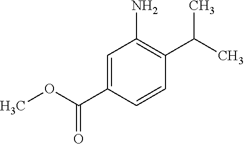

The title compound was prepared from methyl 3-nitro-4-(prop-1-en-2-yl)benzoate (C264) and was isolated as a brown liquid (3.3 g, 91%): ESIMS m/z 194 ([M+H]⁺).

### 5-Methoxy-2-(3,3,3-trifluoropropoxy)aniline (C217)

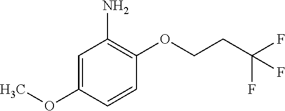 [SMILES: COc1ccc(OCCC(F)(F)F)c(N)c1]

The title compound was prepared from 4-methoxy-2-nitro-1-(3,3,3-trifluoropropoxy)benzene (C302) and was isolated as a dark brown liquid (4.5 g, 84%): ¹H NMR (400 MHz, DMSO-d₆) δ 6.73 (d, J=8.8 Hz, 1H), 6.25 (d, J=2.8 Hz, 1H), 6.05 (dd, J=2.8, 8.4 Hz, 1H), 4.74 (s, 2H), 4.06 (t, J=6.0 Hz, 2H), 3.62 (s, 3H), 2.82-2.70 (m, 2H); ESIMS m/z 236 ([M+H]⁺).

### 1-(3-Amino-4-isopropylphenyl)ethan-1-one (C218)

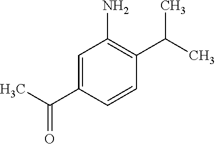

The title compound was prepared from 1-(3-nitro-4-(prop-1-en-2-yl)phenyl)ethan-1-one (C265) and was isolated as a dark brown liquid (2.7 g, 30%): ¹H NMR (400 MHz, DMSO-d₆) δ 7.20 (d, J=2.0, Hz, 1H), 7.17-7.10 (m, 2H), 5.12 (s, 2H), 3.03-2.97 (m, 1H), 2.46 (s, 3H), 1.15 (d, J=6.8 Hz, 6H); ESIMS m/z 178.24 ([M+H]⁺).

### tert-Butyl (3-amino-4-isopropylphenyl)(methyl)carbamate (C219)

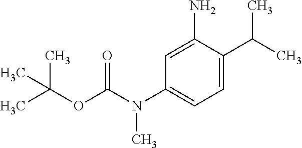 [SMILES: CC(C)c1ccc(N(C)C(=O)OC(C)(C)C)cc1N]

The title compound was prepared from tert-butyl methyl(3-nitro-4-(prop-1-en-2-yl)phenyl)carbamate (C269) and was isolated as an off-white solid (6.0 g, 83%): ¹H NMR (400 MHz, DMSO-d₆) δ 6.92 (d, J=8.3 Hz, 1H), 6.50 (d, J=2.0 Hz, 1H), 6.39 (dd, J=2.4, 8.4 Hz, 1H), 4.88 (s, 2H), 3.08 (s, 3H), 2.93-2.89 (m, 1H), 1.38 (s, 9H), 1.12 (d, J=6.8 Hz, 6H); ESIMS m/z 265 ([M+H]⁺).

### tert-Butyl (3-amino-4-isopropylphenyl)(ethyl)carbamate (C220)

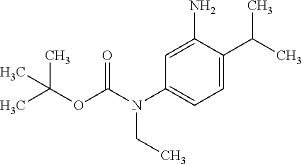 [SMILES: CCN(C(=O)OC(C)(C)C)c1ccc(C(C)C)c(N)c1]

The title compound was prepared from tert-butyl ethyl(3-nitro-4-(prop-1-en-2-yl)phenyl)carbamate (C270) and was isolated as an off-white solid (9.0 g, 90%): ¹H NMR (400 MHz, DMSO-d₆) δ 6.93 (d, J=8.4 Hz, 1H), 6.46 (d, J=2.0 Hz, 1H), 6.34 (dd, J=2.0, 8.4 Hz, 1H), 4.89 (s, 2H), 3.48 (q, J=6.8 Hz, 2H), 2.95-2.85 (m, 1H), 1.37 (s, 9H), 1.13 (d, J=6.8 Hz, 6H), 1.04 (t, J=6.8 Hz, 3H); ESIMS m/z 279 ([M+H]⁺).

### tert-Butyl (3-amino-4-isopropylphenyl)(propyl)carbamate (C221)

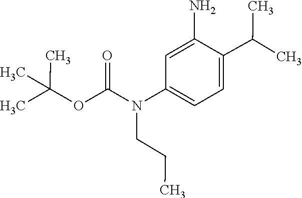 [SMILES: CCCN(C(=O)OC(C)(C)C)c1ccc(C(C)C)c(N)c1]

The title compound was prepared from tert-butyl (3-nitro-4-(prop-1-en-2-yl)phenyl)(propyl)carbamate (C271) and was isolated as an off-white solid (10.0 g, 90%): ¹H NMR (400 MHz, DMSO-d₆) δ 6.93 (d, J=8.0 Hz, 1H), 6.46 (d, J=2.4 Hz, 1H), 6.35 (dd, J=2.0, 7.6 Hz, 1H), 4.89 (s, 2H), 3.42 (t, J=7.2 Hz, 2H), 2.93-2.89 (m, 1H), 1.47-1.40 (m, 2H), 1.37 (s, 9H), 1.13 (d, J=6.8 Hz, 6H), 0.82 (t, J=7.6 Hz, 3H); ESIMS m/z 293 ([M+H]⁺).

### N,N-Dimethyl-4-(trifluoromethyl)benzene-1,3-diamine (C223)

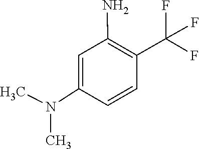

The title compound was prepared from N,N-dimethyl-3-nitro-4-(trifluoromethyl)aniline (C293) and was isolated as a brown liquid (3.0 g, 96%): ¹H NMR (300 MHz, DMSO-d₆) δ 7.08 (d, J=9.0 Hz, 1H), 6.07-6.02 (m, 2H), 5.16 (s, 2H), 2.96 (s, 6H); ESIMS m/z 205 ([M+H]⁺).

### N,N-Dimethyl-4-propylbenzene-1,3-diamine (C224)

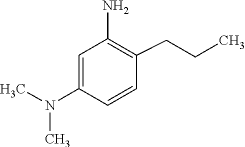

The title compound was prepared from (E)-N,N-dimethyl-3-nitro-4-(prop-1-en-1-yl)aniline (C273) and was isolated as a brown liquid (3.1 g, 98%): ¹H NMR (300 MHz, DMSO-d₆) δ 6.68 (d, J=6.0 Hz, 1H), 6.03 (d, J=2.1 Hz, 1H), 5.93 (dd, J=3.0, 9.0 Hz, 1H), 4.56 (s, 2H), 2.77 (s, 6H), 2.28 (t, J=8.7 Hz, 2H), 1.52-1.42 (m, 2H), 0.88 (t, J=6.0 Hz, 3H); ESIMS m/z 179 ([M+H]⁺).

### N,N-Dimethyl-4-(2,2,2-trifluoroethoxy)benzene-1,3-diamine (C225)

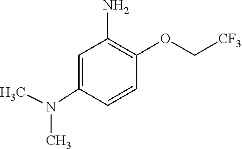 [SMILES: CN(C)c1ccc(OCC(F)(F)F)c(N)c1]

The title compound was prepared from N,N-dimethyl-3-nitro-4-(2,2,2-trifluoroethoxy)aniline (C308) and was isolated as an off-white solid (3 g, 95%): ¹H NMR (400 MHz, CDCl₃) δ 6.74 (d, J=8.8 Hz, 1H), 6.18 (d, J=2.8 Hz, 1H), 6.10 (dd, J=3.2, 8.8 Hz, 1H), 4.27 (q, J=8.4 Hz, 2H), 3.77 (br s, 2H), 2.92 (s, 6H); ESIMS m/z 235 ([M+H]⁺).

### N,N-Diethyl-4-isopropylbenzene-1,3-diamine (C226)

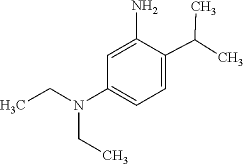

The title compound was prepared from N,N-diethyl-3-nitro-4-(prop-1-en-2-yl)aniline (C267) and was isolated as a brown liquid (6 g, 81%): ¹H NMR (400 MHz, DMSO-d₆) δ 6.75 (d, J=8.4, Hz, 1H), 5.99 (d, J=2.9 Hz, 1H), 5.92 (dd, J=2.8, 8.4 Hz, 1H), 4.53 (br s, 2H), 3.25-3.17 (m, 4H), 2.89-2.76 (m, 1H), 1.09-1.02 (m, 12H); ESIMS m/z 207 ([M+H]⁺).

### 4-Isopropyl-N,N-dipropylbenzene-1,3-diamine (C227)

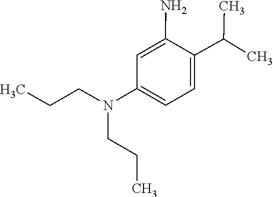 [SMILES: CCCN(CCC)c1ccc(C(C)C)c(N)c1]

The title compound was prepared from 3-nitro-4-(prop-1-en-2-yl)-N,N-dipropylaniline (C268) and was isolated as a brown liquid (4.0 g, 80%): ¹H NMR (400 MHz, DMSO-d₆) δ 6.74 (d, J=8.4 Hz, 1H), 5.96 (d, J=2.4 Hz, 1H), 5.88 (dd, J=2.8, 8.8 Hz, 1H), 4.53 (br s, 2H), 3.10 (t, J=7.6 Hz, 4H), 2.83-2.79 (m, 1H), 1.52-1.45 (m, 4H), 1.08 (d, J=6.8 Hz, 6H), 0.86 (t, J=7.6 Hz, 6H); ESIMS m/z 235 ([M+H]⁺).

### N-(3-Amino-4-isopropylphenyl)-N-methylacetamide (C228)

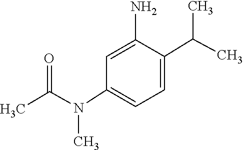

The title compound was prepared from N-methyl-N-(3-nitro-4-(prop-1-en-2-yl)phenyl)acetamide (C257) and was isolated as an off-white solid (1.5 g, 93%): ¹H NMR (400 MHz, CDCl₃) δ 7.13 (d, J=8.1 Hz, 1H), 6.56 (dd, J=8.1, 2.2 Hz, 1H), 6.47 (d, J=2.2 Hz, 1H), 3.21 (s, 3H), 2.88 (p, J=6.8 Hz, 1H), 1.89 (s, 3H), 1.27 (d, J=6.9 Hz, 6H); EIMS m/z 206.

### N-(3-Amino-4-isopropylphenyl)-N-ethylacetamide (C229)

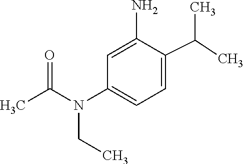 [SMILES: CCN(C(C)=O)c1ccc(C(C)C)c(N)c1]

The title compound was prepared from N-ethyl-N-(3-nitro-4-(prop-1-en-2-yl)phenyl)acetamide (C258) and was isolated as an off-white solid (1.3 g, 92%): ¹H NMR (400 MHz, CDCl₃) δ 7.13 (d, J=8.1 Hz, 1H), 6.53 (dd, J=8.1, 2.2 Hz, 1H), 6.45 (d, J=2.2 Hz, 1H), 3.77-3.65 (m, 2H), 2.88 (p, J=6.8 Hz, 1H), 1.85 (s, 3H), 1.27 (d, J=6.9 Hz, 6H), 1.11 (t, J=7.2 Hz, 3H); EIMS m/z 220.

### 2-(sec-Butoxy)-5-methylaniline (C230)

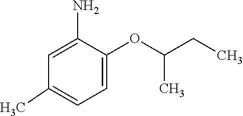

The title compound was prepared from 1-(sec-butoxy)-4-methyl-2-nitrobenzene (C305) using 60 psi of hydrogen and EtOAc as solvent and was isolated as a red-colored oil (4.3 g, 93%): ¹H NMR (300 MHz, CDCl₃) δ 6.67 (d, J=8.1 Hz, 1H), 6.55 (s, 1H), 6.49 (d, J=8.1 Hz, 1H), 4.26-4.20 (m, 1H), 3.72 (br s, 2H), 2.21 (s, 3H), 1.78-1.57 (m, 2H), 1.29-0.95 (m, 3H), 0.98 (t, J=6.6 Hz, 3H); ESIMS m/z 180 ([M+H]⁺).

### 2-(sec-Butyl)-5-methylaniline (C231)

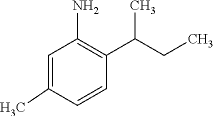

The title compound was prepared from (E)-1-(but-2-en-2-yl)-4-methyl-2-nitrobenzene (C261) and was isolated as a brown liquid that was used without purification (2.8 g).

### 5-Ethoxy-2-propylaniline (C232)

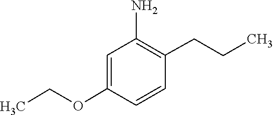

The title compound was prepared from 1-allyl-4-ethoxy-2-nitrobenzene (C276) and was isolated as a brown liquid (859 mg, 78%): ¹H NMR (400 MHz, CDCl₃) δ 6.92 (d, J=8.2 Hz, 1H), 6.33-6.21 (m, 2H), 3.97 (q, J=6.9 Hz, 2H), 3.61 (s, 2H), 2.47-2.35 (m, 2H), 1.69-1.57 (m, 2H), 1.38 (td, J=7.0, 1.6 Hz, 3H), 0.98 (t, J=7.3 Hz, 3H); ¹³C NMR (126 MHz, CDCl₃) δ 158.17, 145.02, 130.20, 119.26, 104.46, 102.01, 63.23, 32.70, 22.20, 14.94, 14.12; EIMS m/z 179.

### 5-Methyl-2-(2,2,2-trifluoro-1-methoxyethyl)aniline (C236)

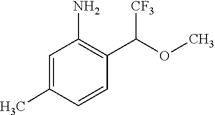

The title compound was prepared from 4-methyl-2-nitro-1-(2,2,2-trifluoro-1-methoxyethyl)benzene (C291) and was isolated as a brown solid (5 g, 95%): ¹H NMR (400 MHz, CDCl₃) δ 7.01 (d, J=8.0 Hz, 1H), 6.59 (d, J=7.6 Hz, 1H), 6.53 (s, 1H), 4.58 (q, J=7.2 Hz, 1H), 3.56 (br s, 2H), 3.46 (s, 3H), 2.26 (s, 3H); ESIMS m/z 220 ([M+H]⁺).

### Example 44: Preparation of 2-butyl-5-methoxyaniline (C233)

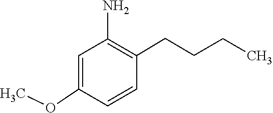

To a stirred solution of 2-bromo-5-methoxyaniline (C253; 5 g, 24.8 mmol) in toluene-water (10:1; 33 mL) were added n-butylboronic acid (6.3 g, 61.9 mmol) and potassium carbonate (8.6 g, 61.9 mmol), and the reaction mixture was degassed with argon for 10 min. Palladium acetate (Pd(OAc)₂; 0.27 g, 1.23 mmol) and tricyclohexanephosphine (0.69 g, 2.47 mmol) were added, the reaction mixture was degassed with argon for 10 min, and the reaction mixture was stirred at 100° C. for 24 h. The reaction mixture was cooled to room temperature, poured into water, and extracted with EtOAc (2×50 mL). The organic layer was washed with brine (50 mL), and the organic layer was dried over anhydrous Na₂SO₄, filtered, and concentrated under reduced pressure. Purification by column chromatography (silica gel 100-200 mesh) eluting with 10-30% EtOAc-petroleum ether afforded the title compound as a greenish-yellow oil (3 g, 68%): ESIMS m/z 180 ([M+H]⁺).

The following compounds were prepared in accordance to the procedure in Example 44.

### 2-Butyl-5-methylaniline (C234)

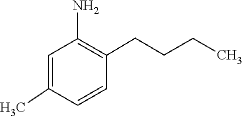

The title compound was prepared from 2-bromo-5-methylaniline and was isolated as a brown liquid that was used without further manipulation (1.3 g): ESIMS m/z 164 ([M+H]⁺).

### 2-Isobutyl-5-methylaniline (C235)

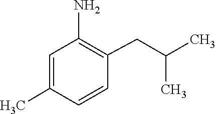

The title compound was prepared from 2-bromo-5-methylaniline and was isolated as a light brown liquid (2.0 g, 66%):¹H NMR (400 MHz, CDCl₃) δ 6.87 (d, J=7.2 Hz, 1H), 6.62-6.48 (m, 2H), 3.52 (s, 2H), 2.33 (d, J=7.2 Hz, 2H), 2.24 (s, 3H), 1.93-1.86 (m, 1H), 0.93 (d, J=6.8 Hz, 6H); ESIMS m/z 164 ([M+H]⁺).

### Example 45: Preparation of 2-isopropyl-5-methoxyaniline (C237)

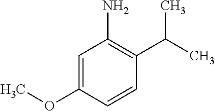

To a 250 mL round-bottomed flask were added 4-methoxy-2-nitro-1-(prop-1-en-2-yl)benzene (C281; 3.72 g, 19.2 mmol), EtOAc (77 mL), and 10% palladium on carbon (2.05 g, 1.92 mmol). The suspension was degassed and backfilled with nitrogen (5×). The suspension was degassed and backfilled with hydrogen. The reaction mixture was stirred at room temperature for 2 h. The suspension was filtered over a pad of diatomaceous earth (which was washed with EtOAc). The filtrate was concentrated under reduced pressure. The title compound was isolated as a clear oil (3.76 g, 91%): ¹H NMR (400 MHz, CDCl₃) δ 7.04 (d, J=8.5 Hz, 1H), 6.35 (dd, J=8.5, 2.6 Hz, 1H), 6.25 (d, J=2.6 Hz, 1H), 3.75 (s, 3H), 3.62 (d, J=38.4 Hz, 2H), 2.83 (hept, J=6.8 Hz, 1H), 1.24 (d, J=6.8 Hz, 6H); ¹³C NMR (101 MHz, CDCl₃) δ 171.10, 158.48, 144.39, 126.22, 125.38, 104.04, 101.65, 60.37, 55.11, 27.18, 22.51, 21.02, 14.20; EIMS m/z 165.

The following compounds were prepared in accordance to the procedure in Example 45.

### 5-Methyl-2-(3,3,3-trifluoropropoxy)aniline (C238)

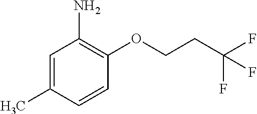

The title compound was prepared from 4-methyl-2-nitro-1-(3,3,3-trifluoropropoxy)benzene (C278) and was isolated as a light yellow oil (1.57 g, 90%): ¹H NMR (400 MHz, CDCl₃) δ 6.67 (d, J=8.1 Hz, 1H), 6.56 (d, J=2.0 Hz, 1H), 6.51 (dd, J=8.1, 2.0 Hz, 1H), 4.20 (t, J=6.3 Hz, 2H), 3.86-3.56 (m, 2H), 2.62 (qt, J=10.5, 6.3 Hz, 2H), 2.22 (s, 3H); ¹⁹F NMR (376 MHz, CDCl₃) δ −64.05-−65.14 (m); EIMS m/z 219.

### 3-Isopropyl-N,N-dimethylbenzene-1,4-diamine (C239)

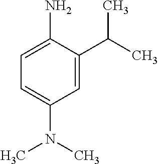

The title compound was prepared from N,N-dimethyl-4-nitro-3-(prop-1-en-2-yl)aniline (C280) and was isolated as a colorless oil (1.06 g, 89%): ¹H NMR (400 MHz, CDCl₃) δ 6.69 (d, J=2.8 Hz, 1H), 6.64 (d, J=8.5 Hz, 1H), 6.56 (dd, J=8.5, 2.8 Hz, 1H), 3.32 (s, 2H), 2.94 (p, J=6.8 Hz, 1H), 2.83 (s, 6H), 1.32-1.18 (m, 6H); EIMS m/z 178.

### 5-Methyl-2-(4,4,4-trifluorobutoxy)aniline (C240)

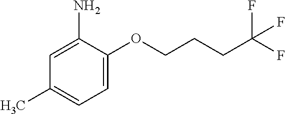 [SMILES: Cc1ccc(OCCCC(F)(F)F)c(N)c1]

The title compound was prepared from 4-methyl-2-nitro-1-(4,4,4-trifluorobutoxy)benzene (C279) and was isolated as a light pink/white solid (3.22 g, 91%): ¹H NMR (400 MHz, CDCl₃) δ 6.65 (d, J=8.1 Hz, 1H), 6.56 (d, J=2.0 Hz, 1H), 6.50 (ddd, J=8.1, 2.1, 0.8 Hz, 1H), 4.02 (t, J=6.0 Hz, 2H), 3.71 (s, 2H), 2.40-2.24 (m, 2H), 2.22 (s, 3H), 2.13-1.99 (m, 2H); ¹⁹F NMR (376 MHz, CDCl₃) δ −66.33; EIMS m/z 233.

### N,N-Dimethyl-4-(3,3,3-trifluoropropoxy)benzene-1,3-diamine (C241)

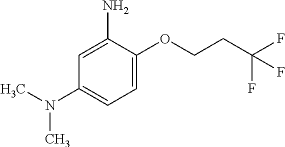 [SMILES: CN(C)c1ccc(OCCC(F)(F)F)c(N)c1]

The title compound was prepared from N,N-dimethyl-3-nitro-4-(3,3,3-trifluoropropoxy)aniline (C277) and was isolated as an off-white solid (1.59 g, 89%): ¹H NMR (400 MHz, CDCl₃) δ 6.72 (d, J=8.7 Hz, 1H), 6.22 (d, J=2.9 Hz, 1H), 6.13 (dd, J=8.8, 2.9 Hz, 1H), 4.16 (t, J=6.4 Hz, 2H), 3.87-3.60 (m, 2H), 2.85 (s, 6H), 2.60 (qt, J=10.6, 6.4 Hz, 2H); ¹⁹F NMR (376 MHz, CDCl₃) δ −64.75; EIMS m/z 248.

### 6-Fluoro-4-isopropyl-N,N-dimethylbenzene-1,3-diamine (C242)

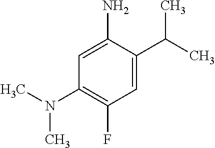

The title compound was prepared from 2-fluoro-N,N-dimethyl-5-nitro-4-(prop-1-en-2-yl)aniline (C284) and was isolated as a colorless oil (1.84 g, 91%): ¹H NMR (300 MHz, CDCl₃) δ 6.80 (d, J=14.3 Hz, 1H), 6.24 (d, J=8.1 Hz, 1H), 3.48 (s, 2H), 2.89-2.79 (m, 1H), 2.78 (d, J=0.7 Hz, 6H), 1.21 (d, J=6.8 Hz, 6H); EIMS m/z 196.

### 2,5-Diisopropylaniline (C243)

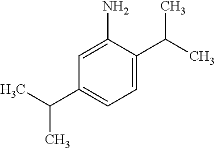

The title compound was prepared from 2-nitro-1,4-di(prop-1-en-2-yl)benzene (C282) and was isolated as a colorless oil (4.7 g, 95%): ¹H NMR (300 MHz, CDCl₃) δ 7.06 (d, J=7.9 Hz, 1H), 6.66 (dd, J=7.8, 1.9 Hz, 1H), 6.56 (d, J=1.9 Hz, 1H), 3.62 (s, 2H), 2.83 (dhept, J=23.6, 6.9 Hz, 2H), 1.27-1.20 (m, 12H); EIMS m/z 177.

### 3-Amino-4-isopropylphenol (C244)

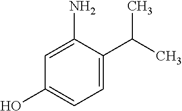

The title compound was prepared from 3-nitro-4-(prop-1-en-2-yl)phenol (C283) and was isolated as a white solid (711 mg, 34%): ¹H NMR (400 MHz, CDCl₃) δ 6.97 (d, J=8.3 Hz, 1H), 6.25 (dd, J=8.3, 2.6 Hz, 1H), 6.18 (d, J=2.6 Hz, 1H), 4.62 (s, 1H), 3.66 (s, 2H), 2.82 (hept, J=6.8 Hz, 1H), 1.22 (d, J=6.8 Hz, 6H); ¹³C NMR (101 MHz, CDCl₃) δ 154.25, 144.50, 126.46, 125.44, 105.78, 102.67, 27.16, 22.51; EIMS m/z 151.

### 5-Ethoxy-2-isopropylaniline (C245)

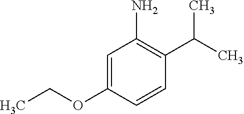

The title compound was prepared from 4-ethoxy-2-nitro-1-(prop-1-en-2-yl)benzene (C275) and was isolated as an orange liquid (322 mg, 93%): ¹H NMR (500 MHz, CDCl₃) δ 7.02 (d, J=8.5 Hz, 1H), 6.34 (dd, J=8.5, 2.5 Hz, 1H), 6.25 (d, J=2.6 Hz, 1H), 3.97 (q, J=7.0 Hz, 2H), 3.51 (s, 2H), 2.83 (hept, J=6.8 Hz, 1H), 1.37 (t, J=7.0 Hz, 3H), 1.23 (d, J=6.8 Hz, 6H); ¹³C NMR (126 MHz, CDCl₃) δ 157.80, 144.33, 126.17, 125.25, 104.69, 102.19, 63.20, 27.16, 22.52, 14.95; EIMS m/z 179.

### 2-Ethyl-5-methoxyaniline (C246)

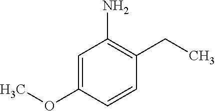

The title compound was prepared from 4-methoxy-1-(1-methoxyethyl)-2-nitrobenzene (C297) and was isolated as an impure oil that was carried on without further manipulation (161 mg).

### Example 46: Preparation of 5-methoxy-2-(trifluoromethyl)aniline (C247)

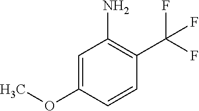

A mixture of 4-methoxy-2-nitro-1-(trifluoromethyl)benzene (C285; 0.97 g, 4.4 mmol) and 10% palladium on carbon (0.467 g, 0.44 mmol) in EtOAc (15 mL) was placed on a Parr shaker under hydrogen for 15 min. The reaction was filtered through diatomaceous earth and concentrated to provide a light yellow liquid. The liquid was loaded onto a diatomaceous earth cartridge with DCM. Purification by flash chromatography (0-50% EtOAc-hexanes) provided the title compound (237 mg). The remaining aniline/nitroso mixture was dissolved in ethanol (15 mL) and catalytic palladium hydroxide was added under a nitrogen atmosphere. The reaction mixture was placed on Parr shaker for 5 h. The reaction mixture was filtered through diatomaceous earth, the cake was rinsed with EtOAc, and the filtrate concentrated. The title compound was isolated as a light yellow liquid (819 mg, 96%): ¹H NMR (400 MHz, CDCl₃) δ 7.34 (d, J=8.8 Hz, 1H), 6.33 (ddt, J=8.8, 2.4, 0.8 Hz, 1H), 6.27-6.17 (m, 1H), 4.19-4.09 (m, 2H), 3.79 (s, 3H); ¹⁹F NMR (376 MHz, CDCl₃) δ −61.25; EIMS m/z 191.

The following compounds were prepared in accordance to the procedure in Example 46.

### 5-Ethoxy-2-(trifluoromethyl)aniline (C248)

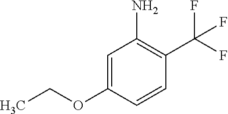

The title compound was prepared from 4-ethoxy-2-nitro-1-(trifluoromethyl)benzene (C286) with 20% palladium hydroxide on carbon in ethanol and was isolated as a brown liquid (748 mg, 93%): ¹H NMR (400 MHz, CDCl₃) δ 7.32 (d, J=8.8 Hz, 1H), 6.31 (ddd, J=8.8, 2.4, 0.8 Hz, 1H), 6.22 (d, J=2.3 Hz, 1H), 4.12 (s, 2H), 4.01 (q, J=7.0 Hz, 2H), 1.40 (t, J=7.0 Hz, 3H); ¹⁹F NMR (376 MHz, CDCl₃) δ −61.20; EIMS m/z 205.

### 5-Methoxy-2-(2,2,2-trifluoroethoxy)aniline (C249)

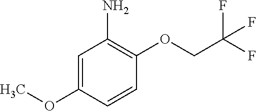

The title compound was prepared from 4-methoxy-2-nitro-1-(2,2,2-trifluoroethoxy)benzene (C287) with 10% palladium on carbon in EtOAc and was isolated as a tan wax (720 mg, 79%): ¹H NMR (400 MHz, CDCl₃) δ 6.73 (d, J=8.8 Hz, 1H), 6.33 (d, J=2.9 Hz, 1H), 6.23 (dd, J=8.8, 2.9 Hz, 1H), 4.30 (q, J=8.3 Hz, 2H), 3.85 (s, 2H), 3.74 (s, 3H); ¹⁹F NMR (376 MHz, CDCl₃) δ −74.30; EIMS m/z 221.

### 4-Fluoro-2-isopropyl-5-methoxyaniline (C250)

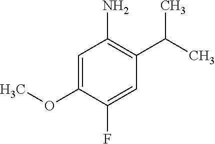

The title compound was prepared from 1-fluoro-2-methoxy-4-nitro-5-(prop-1-en-2-yl)benzene (C274) with 10% palladium on carbon in EtOAc and was isolated as a brown liquid (639 mg, 78%): ¹H NMR (400 MHz, CDCl₃) δ 6.86 (d, J=13.0 Hz, 1H), 6.31 (d, J=7.7 Hz, 1H), 3.83 (s, 3H), 2.90-2.75 (m, 1H), 1.21 (d, J=6.8 Hz, 6H); ¹⁹F NMR (376 MHz, CDCl₃) δ −147.64; EIMS m/z 183.

### 2-(1-Methoxyethyl)-4-methylaniline (C251)

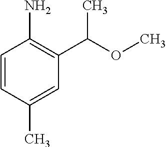

The title compound was prepared from 2-(1-methoxyethyl)-4-methyl-1-nitrobenzene (C288) with 10% palladium on carbon in EtOAc and was isolated as an orange liquid (407 mg, 90%): ¹H NMR (400 MHz, CDCl₃) δ 6.93-6.84 (m, 1H), 6.80 (d, J=2.1 Hz, 1H), 6.56 (d, J=8.0 Hz, 1H), 4.34 (q, J=6.6 Hz, 1H), 4.13 (s, 2H), 3.27 (s, 3H), 2.23 (s, 3H), 1.52 (d, J=6.7 Hz, 3H); ¹³C NMR (126 MHz, CDCl₃) δ 142.37, 129.13, 128.87, 126.94, 125.53, 116.47, 80.61, 56.11, 20.40, 19.99; EIMS m/z 165.

### Example 47: Preparation of 5-methoxy-2-(1-methoxyethyl)aniline (C252)

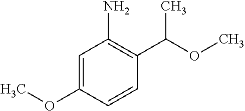

To tert-butyl (5-methoxy-2-(1-methoxyethyl)phenyl)carbamate (C256; 166 mg, 0.59 mmol) was added tetra-n-butyl ammonium fluoride (1 M in THF; 2.4 mL, 2.4 mmol), and the reaction was heated at 55° C. for 3 h. The reaction mixture was cooled and concentrated under nitrogen, and the residue was loaded onto a diatomaceous earth cartridge. Purification by flash chromatography (0-100% EtOAc-hexanes) provided the title compound as a yellow liquid (88 mg, 81%): ¹H NMR (400 MHz, CDCl₃) δ 6.89 (d, J=8.2 Hz, 1H), 6.25 (dd, J=8.3, 2.5 Hz, 1H), 6.19 (d, J=2.5 Hz, 1H), 4.39-4.22 (m, 3H), 3.75 (s, 3H), 3.25 (s, 3H), 1.52 (d, J=6.6 Hz, 3H); EIMS m/z 181.

### Example 48: Preparation of 2-bromo-5-methoxyaniline (C253)

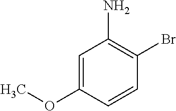

To a stirred solution of 1-bromo-4-methoxy-2-nitrobenzene (14 g, 60.3 mmol) in ethanol-water (3:1; 100 mL) were added iron powder (12.9 g, 241.2 mmol) and ammonium chloride (12.9 g, 241.2 mmol), and the reaction mixture was stirred at 90° C. for 16 h. The reaction mixture was cooled to room temperature and filtered through a pad of diatomaceous earth, which was washed with ethanol. The filtrate was concentrated under reduced pressure. Purification by column chromatography (silica gel 100-200 mesh) eluting with 10-15% EtOAc-petroleum ether afforded the title compound as greenish yellow oil (9 g, 75%): ¹H NMR (300 MHz, CDCl₃) δ 7.28 (s, 1H), 6.32 (d, J=2.7 Hz, 1H), 6.21 (dd, J=2.7, 8.7 Hz, 1H), 4.05 (br s, 2H), 3.74 (s, 3H); ESIMS m/z 202 ([M+H]⁺).

### Example 49: Preparation of 2-(1-(methylthio)ethyl)aniline (C254)

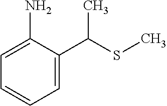

To a magnetically-stirred solution of methyl(1-(2-nitrophenyl)ethyl)sulfane (C298; 5.64 g, 28.6 mmol) and nickel(II) chloride (3.71 g, 28.6 mmol) in dry methanol (143 mL) was added sodium borohydride (4.33 g, 114 mmol, in 200 mg portions) in a dry 500 mL round-bottomed flask under a nitrogen atmosphere. The addition was performed at 0° C. over 1.5 h. The reaction mixture was stirred for an additional 15 min. The solids were removed by vacuum filtration, and the solvent was concentrated. The residue was dissolved in DCM, and the mixture was washed with saturated sodium bicarbonate. The organic layer was dried, filtered, and concentrated. Purification by flash chromatography provided the title compound as a tan oil (3.31 g, 59%): ¹H NMR (400 MHz, CDCl₃) δ 7.14-7.05 (m, 2H), 6.76 (td, J=7.5, 1.3 Hz, 1H), 6.69 (dd, J=7.8, 1.2 Hz, 1H), 4.18 (s, 2H), 3.99 (q, J=7.0 Hz, 1H), 1.93 (s, 3H), 1.68 (d, J=7.1 Hz, 3H).

The following compound was prepared in accordance to the procedure in Example 49.

### 2-(1-Methoxyethyl)aniline (C255)

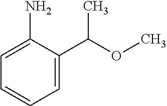

The title compound was prepared from 1-(1-methoxyethyl)-2-nitrobenzene (C290) and was isolated as a tan oil (3.25 g, 40%): ¹H NMR (400 MHz, CDCl₃) δ 7.08 (ddd, J=7.9, 7.3, 1.6 Hz, 1H), 6.99 (dd, J=7.5, 1.5 Hz, 1H), 6.69 (td, J=7.4, 1.2 Hz, 1H), 6.63 (dd, J=8.0, 1.2 Hz, 1H), 4.39 (q, J=6.7 Hz, 1H), 4.33-4.20 (m, 2H), 3.27 (s, 3H), 1.53 (d, J=6.7 Hz, 3H); ¹³C NMR (126 MHz, CDCl₃) δ 145.02, 128.62, 128.43, 125.40, 117.73, 116.28, 80.66, 56.06, 19.84.

### Example 50: Preparation of tert-butyl (5-methoxy-2-(1-methoxyethyl)phenyl)carbamate (C256)

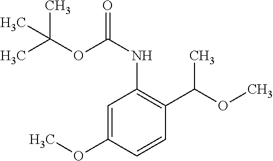 [SMILES: COc1ccc(C(C)OC)c(NC(=O)OC(C)(C)C)c1]

A mixture of 2-bromo-4-methoxy-1-(1-methoxyethyl)benzene (C289; 650 mg, 2.65 mmol) and potassium phosphate (693 mg, 3.98 mmol) in dioxane (6.6 mL) was flushed with nitrogen for 10 min in a microwave vial equipped with a stir bar. tert-Butyl carbamate (466 mg, 3.98 mmol) and chloro(2-dicyclohexylphosphino-2′,6′-diisopropoxy-1,1′-biphenyl)[2-(2′-amino-1,1′-biphenyl)]palladium(II) (RuPhos-Pd-G2, 206 mg, 0.26 mmol) were added. The reaction vial was capped and heated at 100° C. for 48 h. The reaction mixture was filtered through diatomaceous earth. The filtrate was concentrated to give a yellow oil. The oil was loaded onto a diatomaceous earth cartridge with DCM. Purification by flash chromatography (0-20% EtOAc-hexanes) provided the title compound as a clear oil (251 mg, 33%): ¹H NMR (400 MHz, CDCl₃) δ 8.20 (s, 1H), 7.74 (d, J=2.6 Hz, 1H), 6.93 (d, J=8.3 Hz, 1H), 6.51 (dd, J=8.3, 2.6 Hz, 1H), 4.36 (q, J=6.7 Hz, 1H), 3.81 (s, 3H), 3.26 (s, 3H), 1.52 (s, 9H), 1.49 (d, J=6.7 Hz, 3H); EIMS m/z 281.

### Example 51: Preparation of N-methyl-N-(3-nitro-4-(prop-1-en-2-yl)phenyl)acetamide (C257)

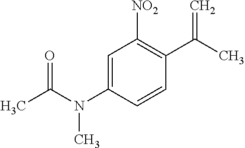 [SMILES: C=C(C)c1ccc(N(C)C(C)=O)cc1[N+](=O)[O-]]

Solid N-(3-nitro-4-(prop-1-en-2-yl)phenyl)acetamide (C259; 1.9 g, 8.63 mmol) was added to a mixture of NaH (60% suspension in mineral oil; 0.414 g, 10.4 mmol) in DMF (34.5 mL). After stirring for 30 min, iodomethane (1.08 mL, 17.3 mmol) was added, and the mixture was stirred overnight. The reaction mixture was poured into water and extracted with ether. The organic extracts were washed with brine and dried. Purification by column chromatography on silica gel eluting with 0-40% acetone-hexanes provided the title compound as an orange solid (1.79 g, 89%): ¹H NMR (400 MHz, CDCl₃) δ 7.73 (s, 1H), 7.40 (s, 2H), 5.23 (s, 1H), 4.98 (s, 1H), 3.31 (s, 3H), 2.10 (s, 3H), 1.96 (s, 3H); ESIMS m/z 235 ([M+H]⁺).

The following compound was prepared in accordance to the procedure in Example 51.

### N-Ethyl-N-(3-nitro-4-(prop-1-en-2-yl)phenyl)acetamide (C258)

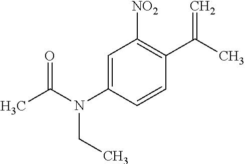 [SMILES: C=C(C)c1ccc(N(CC)C(C)=O)cc1[N+](=O)[O-]]

The title compound was prepared from solid N-(3-nitro-4-(prop-1-en-2-yl)phenyl)acetamide (C259) and was isolated as a yellow powder (1.62 g, 76%): ¹H NMR (400 MHz, CDCl₃) δ 7.69 (s, 1H), 7.44-7.35 (m, 2H), 5.23 (s, 1H), 4.99 (s, 1H), 3.78 (q, J=7.1 Hz, 2H), 2.13-2.09 (m, 3H), 1.15 (t, J=7.2 Hz, 3H), 1.90 (s, 3H); ESIMS m/z 249 ([M+H]⁺).

### Example 52: Preparation of N-(3-nitro-4-(prop-1-en-2-yl)phenyl)acetamide (C259)

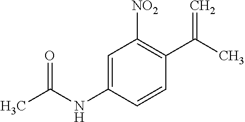 [SMILES: C=C(C)c1ccc(NC(C)=O)cc1[N+](=O)[O-]]

3-Nitro-4-(prop-1-en-2-yl)aniline (C260; 3.7 g, 20.8 mmol), pyridine (3.36 mL, 41.5 mmol), and N,N-dimethylpyridin-4-amine (0.254 g, 2.08 mmol) were dissolved in DCM (104 mL). Acetic anhydride (2.94 mL, 31.1 mmol) was added slowly. The reaction mixture was stirred at room temperature for 3 h. The solvent was removed under a stream of nitrogen to afford a red, crystalline solid. The solid was taken up in DCM and washed with water and brine. The layers were separated, and the organic extracts were concentrated. Purification by silica gel column chromatography eluting with 0-50% acetone-hexanes yielded the title compound as an off-yellow powder (3.88 g, 85%): ¹H NMR (400 MHz, CDCl₃) δ 8.03 (d, J=2.3 Hz, 1H), 7.73 (dd, J=8.4, 2.3 Hz, 1H), 7.32 (s, 1H), 7.28 (d, J=8.4 Hz, 1H), 5.16 (q, J=1.5 Hz, 1H), 4.93 (q, J=1.1 Hz, 1H), 2.22 (s, 3H), 2.06 (dd, J=1.6, 1.0 Hz, 3H).

### Example 53: Preparation of 3-nitro-4-(prop-1-en-2-yl)aniline (C260)

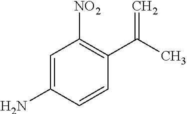

4-Bromo-3-nitroaniline (5 g, 23 mmol), 4,4,5,5-tetramethyl-2-(prop-1-en-2-yl)-1,3,2-dioxaborolane (5.20 mL, 27.6 mmol), sodium carbonate (2.93 g, 27.6 mmol), and Pd(PPh₃)₂Cl₂ (0.485 g, 0.691 mmol) were combined in a flask with dioxane (56 mL) and water (14 mL). The mixture was purged of oxygen and heated to 90° C. for 8 h. The reaction mixture was poured into water and extracted with ether. The organic extracts were dried over Na₂SO₄ and concentrated. Purification by silica gel column chromatography eluting with 0-35% acetone-hexanes afforded the title compound as a red/orange oil that solidified upon standing (3.70 g, 90%): ¹H NMR (300 MHz, CDCl₃) δ 7.12 (d, J=2.5 Hz, 1H), 7.09 (d, J=8.3 Hz, 1H), 6.81 (dd, J=8.3, 2.5 Hz, 1H), 5.10 (p, J=1.6 Hz, 1H), 4.88 (dd, J=1.7, 0.9 Hz, 1H), 3.91 (s, 2H), 2.02 (dd, J=1.5, 0.9 Hz, 3H); EIMS m/z 178.

### Example 54: Preparation of (E)-1-(but-2-en-2-yl)-4-methyl-2-nitrobenzene (C261)

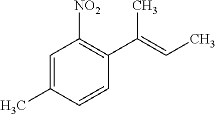

To a suspension of 1-bromo-4-methyl-2-nitrobenzene (3.5 g, 16.2 mmol) in DMF (50 mL), degassed with argon, were added (Z)-tributyl(1-methyl-1-propenyl)stannane (6.0 g, 24.4 mmol), cesium fluoride (4.92 g, 32.4 mmol), and Pd(PPh₃)₂Cl₂ (1.13 g, 1.62 mmol), and the reaction mixture was stirred at 80° C. for 16 h. The reaction mixture was cooled to room temperature, poured into water and extracted with EtOAc (2×50 mL). The organic layer was washed with brine, dried over Na₂SO₄, filtered, and concentrated under reduced pressure. Purification of the residue by column chromatography (silica gel 100-200 mesh) eluting with 10-20% EtOAc-petroleum ether afforded the title compound as a brown oil (1.7 g, 54%): ¹H NMR (400 MHz, CDCl₃) δ 7.61 (s, 1H), 7.30 (d, J=8.0 Hz, 1H), 7.15 (dd, J=7.6 Hz, 1H), 5.44 (q, J=1.2 Hz, 1H), 2.39 (s, 3H), 1.91 (s, 3H), 1.74 (d, J=6.6 Hz, 3H); ESIMS m/z 192 ([M+H]⁺).

The following compound was prepared in accordance to the procedure in Example 54.

### (E)-1-(But-2-en-2-yl)-4-methoxy-2-nitrobenzene C₂₆₂)

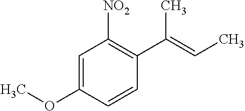

The title compound was prepared from 1-bromo-4-methoxy-2-nitrobenzene and (Z)-tributyl(1-methyl-1-propenyl)stannane and was isolated as a brown oil (1.5 g, 60%): ¹H NMR (300 MHz, CDCl₃) δ 7.32 (d, J=2.7 Hz, 1H), 7.17 (d, J=8.4 Hz, 1H), 7.05 (dd, J=3.3, 8.4 Hz, 1H), 5.42 (q, J=1.5 Hz, 1H), 3.85 (s, 3H), 1.90 (s, 3H), 1.73 (d, J=6.6 Hz, 3H); ESIMS m/z 208 ([M+H]⁺).

### Example 55: Preparation of 3-nitro-4-(prop-1-en-2-yl)benzonitrile (C263)

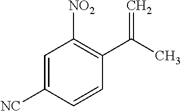

To an argon-degassed solution of 4-bromo-3-nitrobenzonitrile (15 g, 66.4 mmol) and 4,4,5,5-tetramethyl-2-(prop-1-en-2-yl)-1,3,2-dioxaborolane (16.7 g, 99.6 mmol) in 1,4-dioxane-water (4:1 ratio; 180 mL) were added sodium carbonate (9.1 g, 86.3 mmol) and bis(triphenylphosphinepalladium(II) dichloride (Pd(PPh₃)₂Cl₂; 3.7 g, 5.30 mmol), and the reaction mixture was stirred at 110° C. for 16 h. The reaction mixture was cooled to room temperature, poured into water (150 mL), and extracted with EtOAc (2×300 mL). The organic layer was dried over anhydrous Na₂SO₄, filtered, and concentrated under reduced pressure. Purification by column chromatography (silica gel 100-200 mesh) eluting with 0-10% EtOAc-petroleum ether afforded the title compound as a pale yellow solid (10.0 g, 80%): ¹H NMR (400 MHz, DMSO-d₆) δ 8.53 (s, 1H) 8.17 (d, J=8.0 Hz, 1H), 7.72 (d, J=8.0 Hz, 1H), 5.29 (s, 1H) 4.99 (s, 1H) 2.06 (s, 3H); ¹³C NMR (101 MHz, DMSO-d₆) δ 148.18, 141.64, 140.55, 136.22, 131.40, 127.87, 117.08, 116.84, 111.37, 22.44; ESIMS m/z 188 ([M]⁻).

The following compounds were prepared in accordance to the procedure in Example 55.

### Methyl 3-nitro-4-(prop-1-en-2-yl)benzoate (C264)

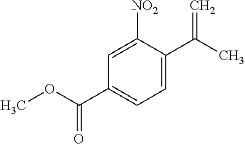 [SMILES: C=C(C)c1ccc(C(=O)OC)cc1[N+](=O)[O-]]

The title compound was prepared from methyl 4-bromo-3-nitrobenzoate and was isolated as a pale yellow liquid (4.0 g, 47%): ¹H NMR (400 MHz, DMSO-d₆) δ 8.38 (d, J=1.2 Hz, 1H), 8.20 (dd, J=1.6, 8.4 Hz, 1H), 7.65 (d, J=8.4 Hz, 1H), 5.27 (s, 1H), 4.97 (s, 1H), 3.91 (s, 3H), 2.06 (s, 3H); ESIMS m/z 222 ([M+H]⁺).

### 1-(3-Nitro-4-(prop-1-en-2-yl)phenyl)ethan-1-one (C265)

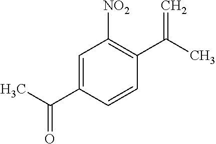

The title compound was prepared from 1-(4-bromo-3-nitrophenyl)ethan-1-one and was isolated as a dark brown liquid (10.0 g, 78%): ¹H NMR (400 MHz, DMSO-d₆) δ 8.39 (d, J=1.6 Hz, 1H), 8.21 (dd, J=1.6, 8.4 Hz, 1H), 7.65 (d, J=7.8 Hz, 1H), 5.27 (s, 1H), 4.97 (s, 1H), 2.65 (s, 3H), 2.06 (s, 3H); ESIMS m/z 206 ([M+H]⁺).

### Example 56: Preparation of N,N-dimethyl-3-nitro-4-(prop-1-en-2-yl)aniline (C266)

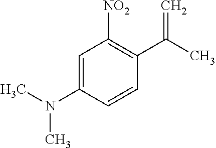

To an argon degassed solution of 4-bromo-N,N-dimethyl-3-nitroaniline (C292; 8.5 g, 34.8 mmol), 4,4,5,5-tetramethyl-2-(prop-1-en-2-yl)-1,3,2-dioxaborolane (8.8 g, 52.2 mmol) in 1,4-dioxane-water (4:1; 130 mL) were added sodium carbonate (4.65 g, 45.2 mmol) and Pd(PPh₃)₂Cl₂ (1.9 g, 2.78 mmol). The reaction mixture was degassed with argon for 5 min and was stirred at 110° C. for 16 h. The reaction mixture was cooled to room temperature, poured into water (150 mL), and extracted with EtOAc (2×300 mL). The organic layer was dried over anhydrous Na₂SO₄, filtered, and concentrated under reduced pressure. Purification by column chromatography (silica gel 100-200 mesh) eluting with 0-10% EtOAc-petroleum ether afforded the title compound as a brown liquid (6.0 g, 83%): ¹H NMR (400 MHz, DMSO-d₆) δ 7.23 (d, J=8.8 Hz, 1H), 7.05 (d, J=2.4 Hz, 1H), 6.96 (dd, J=2.8, 8.8 Hz, 1H), 5.07-5.06 (m, 1H), 4.83-4.80 (m, 1H), 2.96 (s, 6H), 1.97 (s, 3H); ESIMS m/z 207 ([M+H]⁺).

The following compounds were prepared in accordance to the procedure in Example 56.

### N,N-Diethyl-3-nitro-4-(prop-1-en-2-yl)aniline (C267)

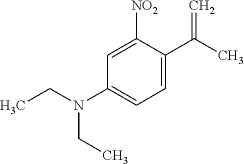 [SMILES: C=C(C)c1ccc(N(CC)CC)cc1[N+](=O)[O-]]

The title compound was prepared from 4-bromo-N,N-diethyl-3-nitroaniline (C294) and was isolated as a brown liquid (8.0 g, 90%): ¹H NMR (400 MHz, DMSO-d₆) δ 7.20 (d, J=8.4 Hz, 1H), 6.98 (d, J=3.2 Hz, 1H), 6.89 (dd, J=3.2, 8.8 Hz, 1H), 5.05 (s, 1H), 4.80 (s, 1H), 3.41-3.36 (m, 4H), 1.96 (s, 3H), 1.09 (t, J=7.2 Hz, 6H); ESIMS m/z 235 ([M+H]⁺).

### 3-Nitro-4-(prop-1-en-2-yl)-N,N-dipropylaniline (C268)

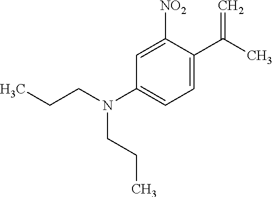 [SMILES: C=C(C)c1ccc(N(CCC)CCC)cc1[N+](=O)[O-]]

The title compound was prepared from 4-bromo-3-nitro-N,N-dipropylaniline (C295) and was isolated as a brown liquid (5.6 g, 86%): ¹H NMR (400 MHz, DMSO-d₆) δ 7.18 (d, J=8.4 Hz, 1H), 6.96 (d, J=2.8 Hz, 1H), 6.87 (dd, J=2.4, 8.4 Hz, 1H), 5.04 (s, 1H), 4.79 (s, 1H), 3.31-3.24 (m, 4H), 1.95 (s, 3H), 1.58-1.48 (m, 4H), 0.89 (t, J=7.2 Hz, 6H); ESIMS m/z 263 ([M+H]⁺).

### tert-Butyl methyl(3-nitro-4-(prop-1-en-2-yl)phenyl)carbamate (C269)

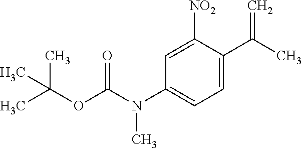 [SMILES: C=C(C)c1ccc(N(C)C(=O)OC(C)(C)C)cc1[N+](=O)[O-]]

The title compound was prepared from tert-butyl (4-bromo-3-nitrophenyl) (methyl)carbamate (C312) and was isolated as a pale yellow liquid (8.0 g, 90%): ¹H NMR (400 MHz, DMSO-d₆) δ 7.89 (d, J=2.4 Hz, 1H), 7.61 (dd, J=2.4, 8.8 Hz, 1H), 7.42 (d, J=8.4 Hz, 1H), 5.19 (s, 1H), 4.90 (s, 1H), 3.24 (s, 3H), 2.02 (s, 3H), 1.43 (s, 9H); ESIMS m/z 237 ([M-(C(CH₃)₃)+H]⁺).

### tert-Butyl ethyl(3-nitro-4-(prop-1-en-2-yl)phenyl)carbamate (C270)

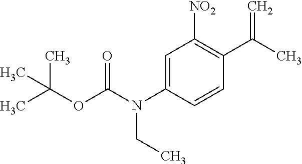 [SMILES: C=C(C)c1ccc(N(CC)C(=O)OC(C)(C)C)cc1[N+](=O)[O-]]

The title compound was prepared from tert-butyl (4-bromo-3-nitrophenyl)(ethyl)carbamate (C313) and was isolated as a pale yellow liquid (11.0 g, 61%): ¹H NMR (400 MHz, DMSO-d₆) δ 7.83 (d, J=2.4 Hz, 1H), 7.57 (d, J=2.0, 8.4 Hz, 1H), 7.43 (d, J=8.4 Hz, 1H), 5.19 (s, 1H), 4.91 (s, 1H), 3.69 (q, J=6.8 Hz, 2H), 2.03 (s, 3H), 1.42 (s, 9H), 1.09 (t, J=7.6 Hz, 3H); ESIMS m/z 251 ([M-(C(CH₃)₃)+H]⁺).

### tert-Butyl (3-nitro-4-(prop-1-en-2-yl)phenyl)(propyl)carbamate (C271)

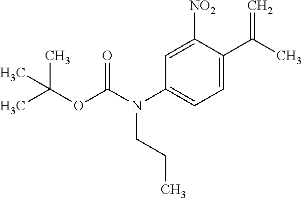 [SMILES: C=C(C)c1ccc(N(CCC)C(=O)OC(C)(C)C)cc1[N+](=O)[O-]]

The title compound was prepared from tert-butyl (4-bromo-3-nitrophenyl)(propyl)carbamate (C314) and was isolated as a pale yellow liquid (12.0 g, 89%): ¹H NMR (400 MHz, DMSO-d₆) δ 7.83 (d, J=2.4 Hz, 1H), 7.58 (dd, J=2.0, 8.4 Hz, 1H), 7.43 (d, J=8.0 Hz, 1H), 5.19 (s, 1H), 4.91 (s, 1H), 3.63 (t, J=7.2 Hz, 2H), 2.03 (s, 3H), 1.50-1.44 (m, 2H), 1.41 (s, 9H), 0.83 (t, J=7.2 Hz, 3H); ESIMS m/z 321.34 ([M+H]⁺).

### Example 57: Preparation of (E)-N,N-dimethyl-3-nitro-4-(prop-1-en-1-yl)aniline (C273)

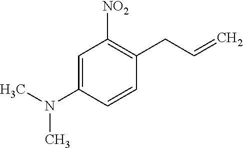

To an argon-degassed solution of 4-bromo-N,N-dimethyl-3-nitroaniline (4.5 g, 18.4 mmol) and allyltributylstannane (9.1 g, 27.55 mmol) in 1,4-dioxane (50 mL) was added Pd(PPh₃)₂Cl₂ (1.28 g, 1.83 mmol), and the reaction mixture was stirred at 120° C. for 16 h. The reaction mixture was cooled to room temperature, poured into water (30 mL), and extracted with EtOAc (2×50 mL). The organic layer was dried over anhydrous Na₂SO₄, filtered, and concentrated under reduced pressure. Purification by column chromatography (silica gel 100-200 mesh) eluting with 0-10% EtOAc-petroleum ether afforded the title compound as a brown liquid (3.5 g, 92%): ¹H NMR (300 MHz, DMSO-d₆) δ 7.23 (d, J=9.0 Hz, 1H), 7.12 (d, J=3.0 Hz, 1H), 6.99 (dd, J=3.0, 6.0 Hz, 1H), 5.96-5.82 (m, 1H), 5.02-4.92 (m, 2H), 3.45 (d, J=9.0 Hz, 2H), 2.94 (s, 6H); ESIMS m/z 207 ([M+H]⁺).

### Example 58: Preparation of 1-fluoro-2-methoxy-4-nitro-5-(prop-1-en-2-yl)benzene (C274)

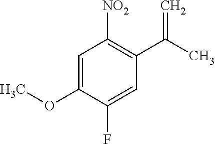

1-Chloro-5-fluoro-4-methoxy-2-nitrobenzene (837 mg, 4.07 mmol), Pd(PPh₃)₂Cl₂ (286 mg, 0.41 mmol), sodium carbonate (518 mg, 4.89 mmol), and 4,4,5,5-tetramethyl-2-(prop-1-en-2-yl)-1,3,2-dioxaborolane (0.918 mL, 4.89 mmol) in dioxane (6.5 mL) and water (1.6 mL) were added to a microwave vial. The reaction mixture was heated in a Biotage® microwave reactor at 140° C. for 30 min. Additional Pd(PPh₃)₂Cl₂ (286 mg, 0.41 mmol) and 4,4,5,5-tetramethyl-2-(prop-1-en-2-yl)-1,3,2-dioxaborolane (400 mg) were added, and the reaction mixture was heated for an additional hour in the Biotage® microwave reactor. The reaction mixture was diluted with EtOAc and washed with water. The organic layer was dried over Na₂SO₄, filtered, and concentrated. Purification by flash chromatography eluting with 0-40% EtOAc-hexanes provided the title compound as a yellow liquid (898 mg, 94%): ¹H NMR (400 MHz, CDCl₃) δ 7.56 (d, J=7.7 Hz, 1H), 7.02 (d, J=11.0 Hz, 1H), 5.17 (p, J=1.5 Hz, 1H), 4.92 (t, J=1.1 Hz, 1H), 3.96 (s, 3H), 2.05 (dd, J=1.5, 0.9 Hz, 3H); ¹⁹F NMR (376 MHz, CDCl₃) δ −125.90.

The following compounds were prepared in accordance to the procedure in Example 58.

### 4-Ethoxy-2-nitro-1-(prop-1-en-2-yl)benzene (C275

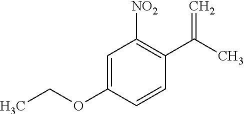

The title compound was prepared from 1-bromo-4-ethoxy-2-nitrobenzene and was isolated as a yellow liquid (384 mg, 56%): ¹H NMR (400 MHz, CDCl₃) δ 7.36 (d, J=2.6 Hz, 1H), 7.21 (d, J=8.5 Hz, 1H), 7.06 (dd, J=8.5, 2.6 Hz, 1H), 5.13 (t, J=1.5 Hz, 1H), 4.90 (dd, J=1.6, 0.9 Hz, 1H), 4.08 (q, J=7.0 Hz, 2H), 2.05 (dd, J=1.5, 0.9 Hz, 3H), 1.44 (t, J=7.0 Hz, 3H); ¹³C NMR (126 MHz, CDCl₃) δ 158.16, 148.61, 142.59, 131.35, 131.12, 119.63, 115.14, 109.19, 64.25, 23.37, 14.60; EIMS m/z 207.

### Example 59: Preparation of 1-allyl-4-ethoxy-2-nitrobenzene (C276)

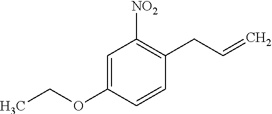

A mixture of 1-chloro-4-ethoxy-2-nitrobenzene (838 mg, 4.16 mmol), allyltributylstannane (1.65 g, 4.99 mmol), and Pd(PPh₃)₂Cl₂ (316 mg, 0.450 mmol) in 1,2-dichloroethane (8 mL) in three microwave reaction vials was reacted in a Biotage microwave reactor at 120° C. for 45 min. The reaction mixture was loaded onto a diatomaceous earth cartridge. Purification by flash chromatography eluting with 0-20% EtOAc-hexanes provided the title compound as a yellow liquid (1.14 g, 100%): ¹H NMR (500 MHz, CDCl₃) δ 7.43 (d, J=2.7 Hz, 1H), 7.24 (d, J=8.5 Hz, 1H), 7.07 (dd, J=8.6, 2.7 Hz, 1H), 5.95 (ddt, J=16.6, 10.1, 6.4 Hz, 1H), 5.17-4.95 (m, 2H), 4.07 (qd, J=7.1, 1.2 Hz, 2H), 3.61 (dt, J=6.4, 1.6 Hz, 2H), 1.44 (td, J=7.0, 3.7 Hz, 3H); ¹³C NMR (126 MHz, CDCl₃) δ 157.72, 135.56, 132.68, 132.34, 126.60, 120.24, 116.61, 109.75, 64.19, 36.35, 14.61; EIMS m/z 207.

### Example 60: Preparation of N,N-dimethyl-3-nitro-4-(3,3,3-trifluoropropoxy)aniline (C277)

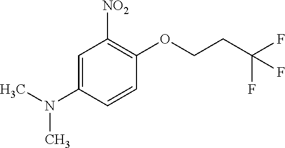 [SMILES: CN(C)c1ccc(OCCC(F)(F)F)c([N+](=O)[O-])c1]

To a 250 mL round-bottomed flask were added THF (21.2 mL), sodium hydride (0.423 mg, 12.7 mmol), and 3,3,3-trifluoropropan-1-ol (0.746 mL, 8.46 mmol). The reaction mixture was stirred at room temperature for 20 min. A solution of 4-fluoro-N,N-dimethyl-3-nitroaniline (C316; 1.641 g, 8.46 mmol) in THF was added slowly. The reaction mixture was stirred for 1 h. The reaction mixture was concentrated under reduced pressure. The remaining slurry was diluted with EtOAc and quenched with water. The mixture was extracted with EtOAc (3×). The organic extracts were combined, washed with brine, dried over Na₂SO₄, filtered, and concentrated under reduced pressure. Purification of the residue by column chromatography afforded the title compound as a dark orange solid (1.95 g, 82%): ¹H NMR (400 MHz, CDCl₃) δ 7.12 (d, J=3.2 Hz, 1H), 7.01 (d, J=9.1 Hz, 1H), 6.87 (dd, J=9.2, 3.2 Hz, 1H), 4.24 (t, J=6.8 Hz, 2H), 2.96 (s, 6H), 2.66 (qt, J=10.5, 6.8 Hz, 2H); ¹⁹F NMR (376 MHz, CDCl₃) δ−64.74, −64.58-−64.87 (m); EIMS m/z 278.

The following compound was prepared in accordance to the procedure in Example 60.

### 4-Methyl-2-nitro-1-(3,3,3-trifluoropropoxy)benzene (C278)

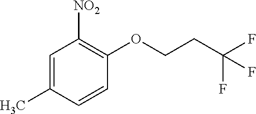 [SMILES: Cc1ccc(OCCC(F)(F)F)c([N+](=O)[O-])c1]

The title compound was prepared from 1-fluoro-4-methyl-2-nitrobenzene and was isolated as a yellow oil (1.88 g, 22%): ¹H NMR (400 MHz, CDCl₃) δ 7.65 (d, J=2.2 Hz, 1H), 7.34 (dd, J=8.5, 2.2 Hz, 1H), 6.97 (d, J=8.5 Hz, 1H), 4.29 (t, J=6.7 Hz, 2H), 2.70 (qt, J=10.4, 6.8 Hz, 2H), 2.36 (s, 3H); ¹⁹F NMR (376 MHz, CDCl₃) δ −64.60, −64.84; EIMS m/z 249.

### 4-Methyl-2-nitro-1-(4,4,4-trifluorobutoxy)benzene (C279)

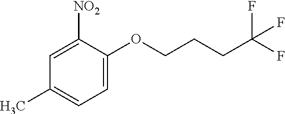 [SMILES: Cc1ccc(OCCCC(F)(F)F)c([N+](=O)[O-])c1]

The title compound was prepared from 1-fluoro-4-methyl-2-nitrobenzene and was isolated as a yellow oil (3.96 g, 55%): ¹H NMR (400 MHz, CDCl₃) δ 7.66 (dd, J=2.2, 0.8 Hz, 1H), 7.32 (ddd, J=8.5, 2.2, 0.8 Hz, 1H), 6.95 (d, J=8.5 Hz, 1H), 4.13 (t, J=5.9 Hz, 2H), 2.52-2.24 (m, 5H), 2.16-1.98 (m, 2H); ¹⁹F NMR (376 MHz, CDCl₃) δ −66.22; EIMS m/z 263.

### Example 61: Preparation of N,N-dimethyl-4-nitro-3-(prop-1-en-2-yl)aniline (C280)

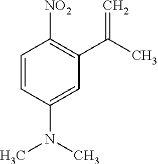

3-Bromo-N,N-dimethyl-4-nitroaniline (1.67 g, 6.81 mmol), sodium carbonate (0.867 g, 8.18 mmol), Pd(PPh₃)₂Cl₂ (0.167 g, 0.238 mmol) were combined in a flask. Dioxane (27.3 mL), water (6.81 mL), and 4,4,5,5-tetramethyl-2-(prop-1-en-2-yl)-1,3,2-dioxaborolane (1.54 mL, 8.18 mmol) were added and the mixture was heated under a nitrogen atmosphere to 80° C. for 10 h. The reaction mixture was concentrated under reduced pressure. The remaining slurry was diluted with EtOAc and water. The mixture was extracted with EtOAc. The organic extracts were combined, washed with brine, dried over Na₂SO₄, filtered, and concentrated under reduced pressure. Purification of the residue by column chromatography afforded the title compound as a bright yellow solid (1.30 g, 91%): ¹H NMR (400 MHz, CDCl₃) δ 8.07 (d, J=9.3 Hz, 1H), 6.54 (dd, J=9.4, 2.9 Hz, 1H), 6.38 (d, J=2.9 Hz, 1H), 5.10 (p, J=1.6 Hz, 1H), 4.90 (dq, J=1.7, 0.8 Hz, 1H), 3.10 (s, 6H), 2.07 (dd, J=1.5, 0.8 Hz, 3H); EIMS m/z 206.

The following compounds were prepared in accordance to the procedure in Example 61.

### 4-Methoxy-2-nitro-1-(prop-1-en-2-yl)benzene (C281)

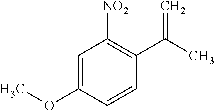

The title compound was prepared from 1-bromo-4-methoxy-2-nitrobenzene and was isolated as a yellow oil (3.72 g, 42%): ¹H NMR (400 MHz, CDCl₃) δ 7.37 (d, J=2.7 Hz, 1H), 7.23 (d, J=8.5 Hz, 1H), 7.08 (dd, J=8.5, 2.6 Hz, 1H), 5.14 (p, J=1.5 Hz, 1H), 4.90 (dt, J=1.8, 0.9 Hz, 1H), 3.86 (s, 3H), 2.05 (dd, J=1.5, 0.9 Hz, 3H); ¹³C NMR (101 MHz, CDCl₃) δ 158.82, 142.52, 131.39, 131.33, 119.19, 115.20, 108.68, 55.84, 23.35; EIMS m/z 193.

### 2-Nitro-1,4-di(prop-1-en-2-yl)benzene (C282)

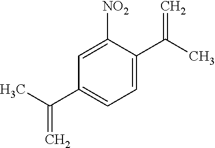

The title compound was prepared from 1,4-dibromo-2-nitrobenzene and was isolated as a yellow oil (3.7 g, 81%): ¹H NMR (300 MHz, CDCl₃) δ 7.92 (d, J=1.9 Hz, 1H), 7.63 (dd, J=8.0, 1.9 Hz, 1H), 7.30-7.27 (m, 1H), 5.47 (q, J=0.9 Hz, 1H), 5.22 (p, J=1.4 Hz, 1H), 5.18 (h, J=1.3 Hz, 1H), 4.95 (p, J=0.9 Hz, 1H), 2.17 (dd, J=1.6, 0.8 Hz, 3H), 2.08 (dd, J=1.6, 0.9 Hz, 3H); EIMS m/z 203.

### 3-Nitro-4-(prop-1-en-2-yl)phenol (C283)

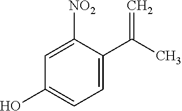

The title compound was prepared from 4-bromo-3-nitrophenol and was isolated as a dark red oil (2.35 g, 28%): ¹H NMR (400 MHz, CDCl₃) δ 7.35 (d, J=2.6 Hz, 1H), 7.19 (d, J=8.4 Hz, 1H), 7.03 (dd, J=8.4, 2.6 Hz, 1H), 5.83 (s, 1H), 5.13 (p, J=1.5 Hz, 1H), 4.90 (dq, J=1.8, 1.0 Hz, 1H), 2.05 (dd, J=1.5, 0.9 Hz, 3H); ESIMS m/z 178 ([M−H]⁺).

### 2-Fluoro-N,N-dimethyl-5-nitro-4-(prop-1-en-2-yl)aniline (C284)

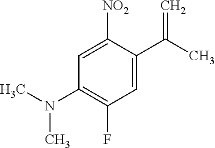 [SMILES: C=C(C)c1cc(F)c(N(C)C)cc1[N+](=O)[O-]]

The title compound was prepared from 4-bromo-2-fluoro-N,N-dimethyl-5-nitroaniline (C311) and was isolated as a bright yellow solid (2.2 g, 80%): ¹H NMR (300 MHz, CDCl₃) δ 7.38 (d, J=8.2 Hz, 1H), 6.93 (d, J=13.2 Hz, 1H), 5.14 (p, J=1.5 Hz, 1H), 4.91 (dt, J=1.4, 0.9 Hz, 1H), 2.92 (d, J=1.1 Hz, 6H), 2.04 (dd, J=1.5, 0.9 Hz, 3H); EIMS m/z 224.

### Example 62: Preparation of 4-methoxy-2-nitro-1-(trifluoromethyl)benzene (C285)

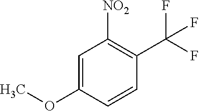

A solution of 1-iodo-4-methoxy-2-nitrobenzene (1.096 g, 3.93 mmol), copper(I) iodide (0.299 g, 1.57 mmol), and methyl 2,2-difluoro-2-(fluorosulfonyl)acetate (2.64 g, 13.8 mmol) in DMF (13 mL) was degassed with nitrogen and was heated at 70° C. overnight. The reaction mixture was cooled, diluted with EtOAc, and poured into a separatory funnel containing saturated aqueous sodium bicarbonate. The organic layer was separated, dried over Na₂SO₄, filtered, and concentrated to provide a golden yellow liquid, which was loaded onto a diatomaceous earth cartridge. Purification by flash chromatography eluting with 0-50% EtOAc-hexanes provided the title compound as a yellow wax (0.80 g, 90%): ¹H NMR (400 MHz, CDCl₃) δ 7.76-7.69 (m, 1H), 7.37 (d, J=2.6 Hz, 1H), 7.17 (ddq, J=8.8, 2.4, 0.9 Hz, 1H), 3.93 (s, 3H); ¹⁹F NMR (376 MHz, CDCl₃) δ−59.00; EIMS m/z 221.

The following compounds were prepared in accordance to the procedure in Example 62.

### 4-Ethoxy-2-nitro-1-(trifluoromethyl)benzene (C286)

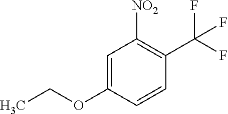 [SMILES: CCOc1ccc(C(F)(F)F)c([N+](=O)[O-])c1]

The title compound was prepared from 4-ethoxy-1-iodo-2-nitrobenzene and was isolated as a yellow liquid (898 mg, 88%): ¹H NMR (500 MHz, CDCl₃) δ 7.71 (d, J=8.8 Hz, 1H), 7.34 (d, J=2.5 Hz, 1H), 7.17-7.10 (m, 1H), 4.14 (q, J=7.0 Hz, 2H), 1.47 (t, J=7.0 Hz, 3H); ¹⁹F NMR (471 MHz, CDCl₃) δ −58.99; EIMS m/z 235.

### Example 63: Preparation of 4-methoxy-2-nitro-1-(2,2,2-trifluoroethoxy)benzene (C287)

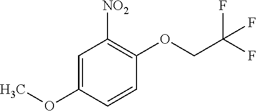 [SMILES: COc1ccc(OCC(F)(F)F)c([N+](=O)[O-])c1]

To 4-methoxy-2-nitrophenol (828 mg, 4.90 mmol) in DMSO (10 mL) was added cesium carbonate (2.07 g, 6.36 mmol). The solution was placed in an ice bath for 5 min. 2,2,2-Trifluoroethyl trifluoromethanesulfonate (2.84 g, 12.2 mmol) was added, and the reaction mixture was stirred at room temperature overnight. The reaction mixture was diluted with EtOAc and washed with water (twice) and brine. The organic layer was dried over Na₂SO₄, filtered, and concentrated. The title compound was isolated as brown waxy needles (1.02 g, 81%): ¹H NMR (400 MHz, CDCl₃) δ 7.41 (t, J=1.7 Hz, 1H), 7.12 (d, J=1.7 Hz, 2H), 4.43 (q, J=8.2 Hz, 2H), 3.85 (s, 3H); ¹⁹F NMR (376 MHz, CDCl₃) δ −74.02.

### Example 64: Preparation of 2-(1-methoxyethyl)-4-methyl-1-nitrobenzene (C288)

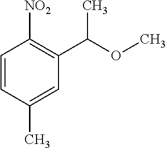

To a vial containing 1-(5-methyl-2-nitrophenyl)ethan-1-01 (C299; 423 mg, 2.34 mmol) and dry THF (4.7 mL) at 0° C. was added NaH (60% suspension in mineral oil; 121 mg, 3.03 mmol). The mixture was stirred at 0° C. for 30 min, and iodomethane (0.19 mL, 3.03 mmol) was added. The reaction mixture was allowed to warm to room temperature for 2 h. The reaction mixture was quenched with saturated aqueous ammonium chloride and EtOAc was added. The biphasic mixture was filtered through a universal phase separator and concentrated. The title compound was isolated as a brown liquid (517 mg, 100%): ¹H NMR (400 MHz, CDCl₃) δ 7.87 (d, J=8.3 Hz, 1H), 7.57-7.50 (m, 1H), 7.23-7.14 (m, 1H), 4.93 (q, J=6.3 Hz, 1H), 3.22 (s, 3H), 2.46 (s, 3H), 1.51 (d, J=6.3 Hz, 3H); ¹³C NMR (126 MHz, CDCl₃) δ 146.27, 144.93, 139.89, 128.57, 127.74, 124.58, 74.99, 56.97, 23.41, 21.68; EIMS m/z 195.

The following compound was prepared in accordance to the procedure in Example 64.

### 2-Bromo-4-methoxy-1-(1-methoxyethyl)benzene (C289)

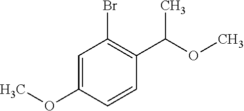

The title compound was prepared from 1-(2-bromo-4-methoxyphenyl)ethan-1-ol (C309) and was isolated as a yellow oil (1.03 g, 98%): ¹H NMR (400 MHz, CDCl₃) δ 7.36 (d, J=8.7 Hz, 1H), 7.07 (d, J=2.6 Hz, 1H), 6.91 (dd, J=8.7, 2.6 Hz, 1H), 4.66 (q, J=6.4 Hz, 1H), 3.80 (s, 3H), 3.23 (s, 3H), 1.38 (d, J=6.4 Hz, 3H); ¹³C NMR (151 MHz, CDCl₃) δ 159.13, 134.52, 127.50, 122.91, 117.55, 114.23, 77.72, 56.56, 55.54, 22.69; EIMS m/z 244, 246.

### 1-(1-Methoxyethyl)-2-nitrobenzene (C290)

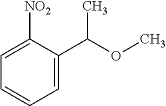

The title compound was prepared from 1-(2-nitrophenyl)ethanol and was isolated as a brown oil (8.19 g, 92%): ¹H NMR (500 MHz, CDCl₃) δ 7.91 (dd, J=8.1, 1.4 Hz, 1H), 7.75 (dd, J=8.0, 1.5 Hz, 1H), 7.66 (td, J=7.6, 1.4 Hz, 1H), 7.42 (ddd, J=8.4, 7.1, 1.6 Hz, 1H), 4.89 (q, J=6.3 Hz, 1H), 3.21 (s, 3H), 1.52 (d, J=6.4 Hz, 3H); ¹³C NMR (126 MHz, CDCl₃) δ 139.61, 133.62, 133.58, 128.01, 127.54, 124.22, 74.97, 56.93, 23.42.

### Example 65: Preparation of 4-methyl-2-nitro-1-(2,2,2-trifluoro-1-methoxyethyl)benzene (C291)

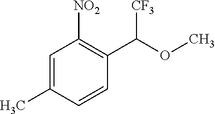

To a suspension of NaH (60% suspension in mineral oil; 0.7 g, 30.6 mmol) in THF (60 mL) was added 2,2,2-trifluoro-1-(4-methyl-2-nitrophenyl)ethan-1-ol (C301; 6 g, 25.5 mmol) at 0° C., and the reaction mixture was stirred at the same temperature for 30 min. Iodomethane (4.3 g, 30.6 mmol) was added, and the reaction mixture was stirred at room temperature for 16 h. The reaction mixture was poured into ice water (50 mL) and was extracted with EtOAc (2×100 mL). The organic layer was washed with water and brine, dried over anhydrous Na₂SO₄, filtered, and concentrated under reduced pressure. Purification by column chromatography (silica gel 100-200 mesh) eluting with 20-40% EtOAc-petroleum ether afforded the title compound as a brown liquid (5 g, 78%): ¹H NMR (400 MHz, CDCl₃) δ 7.84-7.83 (m, 1H), 7.73 (d, J=8.0 Hz, 1H), 7.52-7.50 (m, 1H), 5.64 (q, J=6.0 Hz, 1H), 3.49 (s, 3H), 2.47 (s, 3H).

### Example 66: Preparation of 4-bromo-N,N-dimethyl-3-nitroaniline (C292)

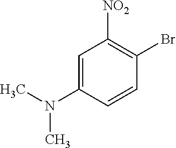

To a stirred suspension of 4-bromo-3-nitroaniline (15 g, 69.12 mmol) in dry DMF (200 mL) was added NaH (60% suspension in mineral oil; 5.3 g, 138.2 mmol) at 0° C. and the reaction mixture was stirred for 30 min. Iodomethane (20.0 g, 138.2 mmol) was added drop-wise, and the reaction mixture was stirred at room temperature for 16 h. The reaction mixture was quenched with ice-water and extracted with EtOAc (3×300 mL). The organic layer was dried over anhydrous Na₂SO₄ and concentrated under reduced pressure. Purification by column chromatography on silica gel (100-200) eluting with 10-15% EtOAc-petroleum ether afforded the title compound as an orange color solid (8.5 g, 51%): ¹H NMR (400 MHz, DMSO-d₆) δ 7.56 (d, J=8.8 Hz, 1H), 7.20 (d, J=2.8 Hz, 1H), 6.88 (dd, J=3.2, 9.2 Hz, 1H), 2.95 (s, 6H); ESIMS m/z 245 ([M+H]⁺).

The following compounds were prepared in accordance to the procedure in Example 66.

### N,N-Dimethyl-3-nitro-4-(trifluoromethyl)aniline (C293)

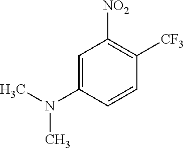 [SMILES: CN(C)c1ccc(C(F)(F)F)c([N+](=O)[O-])c1]

The title compound was prepared from 3-nitro-4-(trifluoromethyl)aniline and was isolated as a brown solid (3.5 g, 62%): ¹H NMR (400 MHz, DMSO-d₆) δ 7.66 (d, J=8.0 Hz, 1H), 7.22 (s, 1H), 6.97 (d, J=8.0 Hz, 1H), 3.04 (s, 6H); ESIMS m/z 235 ([M+H]⁺).

### 4-Bromo-N,N-diethyl-3-nitroaniline (C294)

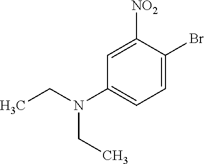

The title compound was prepared from 4-bromo-3-nitroaniline and was isolated as a brown liquid (10.0 g, 53%): ¹H NMR (400 MHz, DMSO-d₆) δ 7.51 (d, J=9.2 Hz, 1H), 7.15 (d, J=2.8 Hz, 1H), 6.84 (dd, J=2.8, 8.8 Hz, 1H), 3.40-3.32 (m, 4H), 1.08 (t, J=6.8 Hz, 6H); ESIMS m/z 273 ([M+H]⁺).

### 4-Bromo-3-nitro-N,N-dipropylaniline (C295)

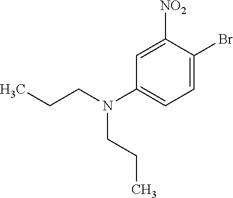 [SMILES: CCCN(CCC)c1ccc(Br)c([N+](=O)[O-])c1]

The title compound was prepared from 4-bromo-3-nitroaniline and was isolated as a brown sticky solid (7.5 g, 36%): ¹H NMR (400 MHz, DMSO-d₆) δ 7.49 (d, J=9.2 Hz, 1H), 7.14 (d, J=3.6 Hz, 1H), 6.82 (dd, J=2.8, 8.8 Hz, 1H), 3.26 (t, J=7.2 Hz, 4H), 1.56-1.46 (m, 4H), 0.88 (t, J=7.6 Hz, 6H); ESIMS m/z 301 ([M+H]⁺).

### Example 67: Preparation of 1-(1-ethoxyethyl)-4-methyl-2-nitrobenzene (C296)

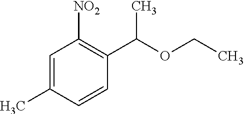

The title compound was prepared from 1-(4-methyl-2-nitrophenyl)ethan-1-ol in a manner similar to that described for the synthesis of 1-(1-methoxyethyl)-4-methyl-2-nitrobenzene in PCT International Application WO 2017040194 A1 and was isolated as a brown liquid (2.9 g, 73%): ¹H NMR (300 MHz, DMSO-d₆) δ 7.75 (s, 1H), 7.62-7.57 (m, 2H), 4.78 (q, J=6.6 Hz, 1H), 3.29-3.13 (m, 2H), 2.39 (s, 3H), 1.39 (d, J=6.6 Hz, 3H), 1.06 (t, J=7.2 Hz, 3H).

The following compound was prepared in accordance to the procedure in Example 67.

### 4-Methoxy-1-(1-methoxyethyl)-2-nitrobenzene (C297)

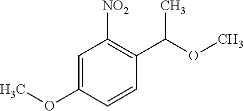

The title compound was prepared from 1-(4-methoxy-2-nitrophenyl)ethan-1-ol (C300) and was isolated as a brown liquid (558 mg, 100%): ¹H NMR (400 MHz, CDCl₃) δ 7.63 (d, J=8.7 Hz, 1H), 7.41 (d, J=2.7 Hz, 1H), 7.20 (dd, J=8.8, 2.7 Hz, 1H), 4.82 (q, J=6.3 Hz, 1H), 3.87 (s, 3H), 3.19 (s, 3H), 1.49 (d, J=6.3 Hz, 3H); ¹³C NMR (126 MHz, CDCl₃) δ 158.83, 149.16, 131.52, 128.60, 120.53, 108.42, 74.66, 56.81, 55.83, 23.45; EIMS m/z 211.

### Example 68: Preparation of methyl(1-(2-nitrophenyl)ethyl)sulfane (C298)

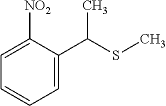

To a magnetically stirred solution of 1-(1-bromoethyl)-2-nitrobenzene (C317; 29 g, 126 mmol) in dry THF (420 mL) at 0° C. was added sodium methanethiolate (9.72 g, 139 mmol) in a dry 1 L round-bottomed flask under nitrogen atmosphere. The reaction mixture was allowed to stir while warming to room temperature overnight. Additional portions of sodium methanethiolate (5 g and 1.2 g) were added, and the reaction mixture was allowed to stir overnight. The mixture was washed with water and with DCM. The combined organic layers were dried over magnesium sulfate and concentracted under reduced pressure. The title compound was isolated as a brown oil (28 g, 96%): ¹H NMR (400 MHz, CDCl₃) δ 7.85-7.80 (m, 1H), 7.75 (dt, J=8.1, 1.0 Hz, 1H), 7.60 (dddd, J=8.0, 7.4, 1.4, 0.7 Hz, 1H), 7.36 (tdd, J=7.3, 1.4, 0.7 Hz, 1H), 4.52 (q, J=6.9 Hz, 1H), 1.91 (d, J=0.9 Hz, 3H), 1.61 (dd, J=7.0, 0.9 Hz, 3H).

### Example 69. Preparation of 1-(5-methyl-2-nitrophenyl)ethan-1-ol (C299)

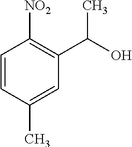

To a dry, 25-mL vial equipped with a stir bar were added 2-iodo-4-methyl-1-nitrobenzene (805 mg, 3.06 mmol) and THF (6 mL). The solution was cooled to −40° C. Phenylmagnesium chloride (1.6 mL, 3.21 mmol) was added in a drop-wise manner. The mixture was stirred for 30 min, and acetaldehyde (0.19 mL, 3.37 mmol) was added in a drop-wise manner. The reaction mixture was stirred at −40° C. for 2 h. Saturated aqueous ammonium chloride (5 mL) and EtOAc (10 mL) were added, and the biphasic mixture was filtered through a universal phase separator. The organic layer was concentrated, and the residue was loaded onto a diatomaceous earth cartridge with DCM. Purification by flash chromatography (0-50% EtOAc-hexanes) provided the title compound as a brown oil (426 mg, 75%): ¹H NMR (400 MHz, CDCl₃) δ 7.86 (d, J=8.3 Hz, 1H), 7.63 (d, J=1.9 Hz, 1H), 7.20 (ddd, J=8.3, 2.0, 0.8 Hz, 1H), 5.45 (qd, J=6.3, 3.7 Hz, 1H), 2.46 (d, J=0.8 Hz, 3H), 2.30 (d, J=3.9 Hz, 1H), 1.57 (d, J=6.3 Hz, 3H); ¹³C NMR (126 MHz, CDCl₃) δ 145.56, 144.98, 141.03, 128.67, 127.94, 124.72, 65.66, 24.13, 21.68; EIMS m/z 181.

The following compound was prepared in accordance to the procedure in Example 69.

### 1-(4-Methoxy-2-nitrophenyl)ethan-1-ol (C300)

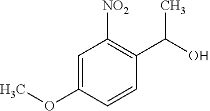

The title compound was prepared from 1-iodo-4-methoxy-2-nitrobenzene and was isolated as a brown oil (105 mg, 71%): ¹H NMR (400 MHz, CDCl₃) δ 7.72 (d, J=8.7 Hz, 1H), 7.40 (d, J=2.7 Hz, 1H), 7.19 (dd, J=8.7, 2.7 Hz, 1H), 5.35 (qd, J=6.3, 3.2 Hz, 1H), 3.87 (s, 3H), 2.27 (d, J=3.7 Hz, 1H), 1.57 (d, J=1.1 Hz, 3H); ¹³C NMR (126 MHz, CDCl₃) δ 158.92, 148.47, 132.84, 128.65, 120.24, 108.74, 65.24, 55.86, 24.02; EIMS m/z 197.

### Example 70: Preparation of 2,2,2-trifluoro-1-(4-methyl-2-nitrophenyl)ethan-1-ol (C301)

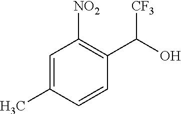 [SMILES: Cc1ccc(C(O)C(F)(F)F)c([N+](=O)[O-])c1]

To a solution of 4-methyl-2-nitrobenzaldehyde (6 g, 36.6 mmol) in THF (70 mL) were added trifluoromethyl trimethylsilane (TMSCF₃; 6.2 g, 43.9 mmol) and tetra-n-butyl ammonium fluoride (1 M in THF; 3.7 mL, 3.66 mmol). The reaction mixture was stirred at room temperature for 4 h, was quenched with concentrated HCl (5 mL), and was stirred for 10 min. Water (50 mL) was added to the mixture and was extracted with EtOAc (2×150 mL). The organic layer was washed with water and brine, dried over anhydrous Na₂SO₄, filtered and concentrated under reduced pressure. Purification by column chromatography (silica gel 100-200 mesh) eluting with 20-40% EtOAc-petroleum ether afforded the title compound as a brown liquid (6 g, 70%): ¹H NMR (400 MHz, CDCl₃) δ 7.82-7.80 (m, 2H), 7.52 (d, J=8.0 Hz, 1H), 6.11-6.07 (m, 1H), 2.98 (br s, 1H), 2.45 (s, 3H).

### Example 71: Preparation of 4-methoxy-2-nitro-1-(3,3,3-trifluoropropoxy)benzene (C302)

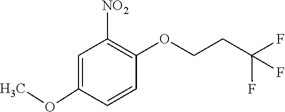 [SMILES: COc1ccc(OCCC(F)(F)F)c([N+](=O)[O-])c1]

To a solution of 4-methoxy-2-nitrophenol (7 g, 61.4 mmol) in acetonitrile (200 mL) were added cesium carbonate (39.9 g, 122.8 mmol) and 1,1,1,3-tetrafluoropropane (10.5 mL, 61.4 mmol) at room temperature, and the reaction mixture was stirred at 60° C. for 16 h. The reaction mixture was cooled to room temperature, was poured into ice water (400 mL), and was extracted with EtOAc (2×200 mL). The organic layer was dried over anhydrous Na₂SO₄, filtered, and concentrated under reduced pressure. Purification by column chromatography (silica gel 100-200 mesh) eluting with 20-30% EtOAc-petroleum ether afforded the title compound as a pale yellow solid (6 g, 46%): mp 59-62° C.; ¹H NMR (400 MHz, DMSO-d₆) δ 7.45 (d, J=2.8 Hz, 1H), 7.36 (d, J=8.8 Hz, 1H), 7.25 (dd, J=3.2, 8.8 Hz, 1H), 4.32 (t, J=6.0 Hz, 2H), 3.78 (s, 3H), 2.81-2.72 (m, 2H); ESIMS m/z 265 ([M]⁺).

The following compound was prepared in accordance to the procedure in Example 71.

### 3-Nitro-4-(3,3,3-trifluoropropoxy)benzonitrile (C303)

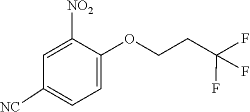 [SMILES: N#Cc1ccc(OCCC(F)(F)F)c([N+](=O)[O-])c1]

The title compound was prepared from 4-hydroxy-3-nitrobenzonitrile and was isolated as a pale yellow solid (8.0 g, 50%): mp 110-113° C.; ¹H NMR (400 MHz, DMSO-d₆) δ 8.50 (d, J=2.4 Hz, 1H), 8.16 (dd, J=2.0, 8.8 Hz, 1H), 7.62 (d, J=8.8 Hz, 1H), 4.51 (t, J=5.6 Hz, 2H), 2.89-2.80 (m, 2H); ESIMS m/z 260 ([M]⁺).

### Example 72: Preparation of 4-methyl-2-nitro-1-propoxybenzene (C304)

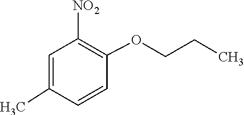

To a solution of 4-methyl-2-nitrophenol (4.0 g, 26.0 mmol) in acetonitrile (50 mL) were added sequentially potassium carbonate (7.1 g, 52.0 mmol) and 1-bromopropane (3.5 mL, 39.0 mmol) drop-wise at 0° C., and the reaction mixture was stirred at 80° C. for 16 h. The reaction mixture was cooled to room temperature, was diluted with water (150 mL), and was extracted with EtOAc (2×200 mL). The organic layer was washed with brine, dried over anhydrous Na₂SO₄, filtered and concentrated under reduced pressure. Purification by column chromatography (silica gel 100-200 mesh) eluting with 10-20% EtOAc-petroleum ether afforded the title compound as a pale yellow liquid (4.0 g, 80%): ¹H NMR (300 MHz, DMSO-d₆) δ 7.67 (s, 1H), 7.46-7.43 (m, 1H), 7.23 (d, J=6.0 Hz, 1H), 4.06 (t, J=6.0 Hz, 2H), 2.29 (s, 3H), 1.71-1.68 (m, 2H), 0.67 (t, J=6.0 Hz, 3H); ESIMS m/z 195 ([M]⁺).

The following compound was prepared in accordance to the procedure in Example 72.

### 1-(sec-Butoxy)-4-methyl-2-nitrobenzene (C305)

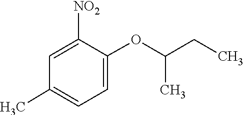

The title compound was prepared from 4-methyl-2-nitrophenol and was isolated as a yellow oil (5.3 g, 77%): ¹H NMR (300 MHz, CDCl₃) δ 7.57 (d, J=1.5 Hz, 1H), 7.29-7.26 (m, 1H), 6.95 (d, J=8.4 Hz, 1H), 4.42-4.36 (m, 1H), 2.33 (s, 3H), 1.82-1.62 (m, 2H), 1.31 (d, J=6.3 Hz, 3H), 0.98 (t, J=6.0 Hz, 3H).

### Example 73: Preparation 1-(ethoxymethyl)-4-methyl-2-nitrobenzene (C306)

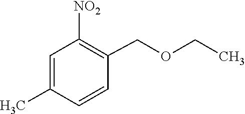

To a solution of (4-methyl-2-nitrophenyl)methanol (C310; 4.0 g, 23.95 mmol) in dry DMF (200 mL) was added NaH (60% suspension in mineral oil; 0.8 g, 35.9 mmol) portion-wise at 0° C., and the reaction mixture was stirred for 30 min. Ethyl iodide (3.8 mL, 47.9 mmol) was added drop-wise, and the reaction mixture was stirred at room temperature for 4 h. The reaction mixture was quenched with ice-water and extracted with EtOAc (2×100 mL). The organic layer was dried over anhydrous Na₂SO₄, filtered, and concentrated under reduced pressure. Purification by column chromatography (silica gel 100-200 mesh) eluting with 5-10% EtOAc-petroleum ether afforded the title compound as a pale yellow liquid (3.8 g, 82%): ¹H NMR (300 MHz, DMSO-d₆) δ 7.85 (s, 1H), 7.58-7.57 (m, 2H), 4.72 (s, 2H), 3.50 (q, J=6.0 Hz, 2H), 2.40 (s, 3H), 1.15 (t, J=6.0 Hz, 3H); ESIMS m/z 208 ([M+H]⁺).

The following compound was prepared in accordance to the procedure in Example 73.

### 4-Methyl-2-nitro-1-((2,2,2-trifluoroethoxy)methyl)benzene (C307)

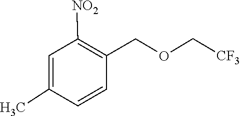 [SMILES: Cc1ccc(COCC(F)(F)F)c([N+](=O)[O-])c1]

The title compound was prepared from (4-methyl-2-nitrophenyl)methanol (C310) using 2,2,2-trifluoroethyl trifluoromethanesulfonate and was isolated as a pale yellow solid (3.5 g, 82%): ¹H NMR (300 MHz, DMSO-d₆) δ 7.91 (s, 1H), 7.78-7.60 (m, 2H), 4.97 (s, 2H), 4.18 (q, J=7.8 Hz, 2H), 2.41 (s, 3H).

### Example 74: Preparation of N,N-dimethyl-3-nitro-4-(2,2,2-trifluoroethoxy)aniline (C308)

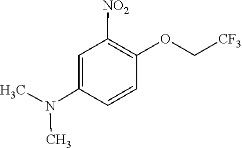 [SMILES: CN(C)c1ccc(OCC(F)(F)F)c([N+](=O)[O-])c1]

To a mixture of NaH (60% suspension in mineral oil; 0.6 g, 2.47 mmol) in THF (50 mL) was added 2,2,2-trifluoroethanol (2.5 g, 2.47 mmol) drop-wise at 0° C. and the reaction mixture was stirred at 0° C. for 30 min. 4-Fluoro-N,N-dimethyl-3-nitroaniline (C316; 3.5 g, 1.90 mmol) in THF (10 mL) was added, and the reaction mixture was stirred at room temperature for 16 h. The reaction mixture was poured into ice water (100 mL) and was extracted with EtOAc (2×150 mL). The organic layer was washed with water and brine, dried over anhydrous Na₂SO₄, filtered, and concentrated under reduced pressure. Purification by column chromatography (silica gel 100-200 mesh) eluting with 20-40% EtOAc-petroleum ether afforded the title compound as a brown solid (3.5 g, 70%): ¹H NMR (400 MHz, CDCl₃) δ 7.13 (d, J=3.2 Hz, 1H), 7.08 (d, J=8.8 Hz, 1H), 6.84 (dd, J=2.8, 8.8 Hz, 1H), 4.38 (q, J=8.4 Hz, 2H), 2.98 (s, 6H); ESIMS m/z 265 ([M+H]⁺).

### Example 75: Preparation of 1-(2-bromo-4-methoxyphenyl)ethan-1-ol (C309)

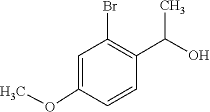

To 2-bromo-4-methoxybenzaldehyde (1.05 g, 4.88 mmol) in anhydrous THF (15 mL) in a dry ice-acetonitrile bath was added methylmagnesium bromide (3 M in diethyl ether; 1.7 mL, 5.1 mmol). After 15 min the reaction mixture was warmed to room temperature and stirred for 2 h. The reaction mixture was quenched with water, diluted with EtOAc, and filtered through a universal phase separator into a tared vial and concentrated under nitrogen. The title compound was isolated as a pale yellow oil (975 mg, 82%): ¹H NMR (400 MHz, CDCl₃) δ 7.48 (d, J=8.7 Hz, 1H), 7.07 (d, J=2.6 Hz, 1H), 6.90 (dd, J=8.7, 2.6 Hz, 1H), 5.20 (qd, J=6.3, 3.0 Hz, 1H), 3.79 (s, 3H), 1.92 (d, J=3.2 Hz, 1H), 1.47 (d, J=6.4 Hz, 3H); ¹³C NMR (126 MHz, CDCl₃) δ 159.20, 136.58, 127.22, 122.07, 117.74, 113.93, 68.75, 55.55, 23.63; EIMS m/z 230, 232.

### Example 76: Preparation of (4-methyl-2-nitrophenyl)methanol (C310)

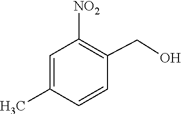

To a solution of 4-methyl-2-nitrobenzoic acid (10.0 g, 52.2 mmol) in THF was added borane-THF complex (1 M in THF; 72 mL, 71.8 mmol) at 0° C., and the reaction mixture was stirred at 70° C. for 4 h. The reaction mixture was cooled to room temperature, was quenched carefully with ice-water (200 mL), and was extracted with EtOAc (2×400 mL). The organic layer was dried over anhydrous Na₂SO₄, filtered, and concentrated under reduced pressure. Purification by column chromatography (silica gel 100-200 mesh) eluting with 10-15% EtOAc-petroleum ether afforded the title compound as a pale yellow solid (8.1 g, 89%): ¹H NMR (300 MHz, DMSO-d₆) δ 7.85 (s, 1H), 7.69 (d, J=8.4 Hz, 1H), 7.57 (d, J=6.0 Hz, 1H), 5.46 (s, 1H), 4.76 (d, J=2.7 Hz, 2H), 2.39 (s, 3H).

### Example 77: Preparation of 4-bromo-2-fluoro-N,N-dimethyl-5-nitroaniline (C311)

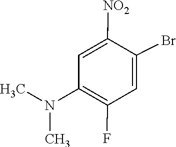

To a 250 mL round-bottomed flask were added 4-bromo-2-fluoro-5-nitroaniline (4 g, 17.0 mmol), THF (85 mL), and sodium hydride (60% dispersion in mineral oil; 1.70 g, 42.6 mmol). The reaction mixture was stirred at room temperature for 10 min, and iodomethane (2.34 mL, 37.4 mmol) was added. The reaction mixture was stirred at room temperature overnight and was concentrated under reduced pressure. The slurry was diluted with EtOAc and water. The mixture was extracted with EtOAc (3×). The organic extracts were combined, washed with brine, dried over Na₂SO₄, filtered, and concentrated under reduced pressure. Purification by column chromatography afforded the title compound as a yellow solid (2.9 g, 62%): ¹H NMR (300 MHz, CDCl₃) δ 7.40-7.28 (m, 2H), 2.94 (d, J=1.4 Hz, 6H); EIMS m/z 262, 264.

### Example 78: Preparation of tert-butyl (4-bromo-3-nitrophenyl) (methyl)carbamate (C312)

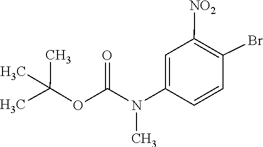 [SMILES: CN(C(=O)OC(C)(C)C)c1ccc(Br)c([N+](=O)[O-])c1]

To a solution of tert-butyl (4-bromo-3-nitrophenyl)carbamate (C315; 10 g, 31.64 mmol) in DMF (150 mL) were added cesium carbonate (20.5 g, 63.1 mmol) and iodomethane (6.8 g, 47.3 mmol) at 0° C. The reaction mixture was stirred at room temperature for 2 h. The reaction mixture was poured into ice water (400 mL) and was extracted with diethyl ether (2×400 mL). The organic layer was dried over anhydrous Na₂SO₄, filtered, and concentrated under reduced pressure. Purification by column chromatography (silica gel 100-200 mesh) eluting with 5-10% EtOAc-petroleum ether afforded the title compound as a pale yellow liquid (10 g, 95%): ¹H NMR (400 MHz, DMSO-d₆) δ 8.03 (d, J=2.8 Hz, 1H), 7.85 (d, J=8.8 Hz, 1H), 7.57 (dd, J=2.8, 8.4 Hz, 1H), 3.23 (s, 3H), 1.43 (s, 9H); ESIMS m/z 275 ([M-(C(CH₃)₃) +H]⁺).

The following compounds were prepared in accordance to the procedure in Example 78.

### tert-Butyl (4-bromo-3-nitrophenyl)(ethyl)carbamate (C313)

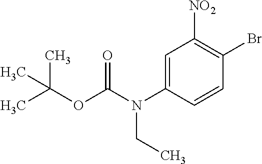 [SMILES: CCN(C(=O)OC(C)(C)C)c1ccc(Br)c([N+](=O)[O-])c1]

The title compound was prepared from tert-butyl (4-bromo-3-nitrophenyl)carbamate (C315) and was isolated as a pale yellow liquid (12 g, 73%): ¹H NMR (400 MHz, DMSO-d₆) δ 7.98 (d, J=2.4 Hz, 1H), 7.86 (d, J=8.8 Hz, 1H), 7.52 (dd, J=2.4, 8.8 Hz, 1H), 3.68 (q, J=6.8 Hz, 2H), 1.41 (s, 9H), 1.08 (t, J=7.2 Hz, 3H); ESIMS m/z 289 ([M-(C(CH₃)₃)+H]⁺).

### tert-Butyl (4-bromo-3-nitrophenyl)(propyl)carbamate (C314)

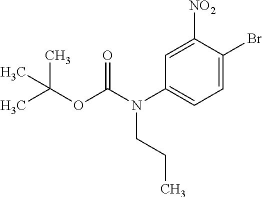 [SMILES: CCCN(C(=O)OC(C)(C)C)c1ccc(Br)c([N+](=O)[O-])c1]

The title compound was prepared from tert-butyl (4-bromo-3-nitrophenyl)carbamate (C315) and was isolated as a pale yellow liquid (15 g, 66%): ¹H NMR (400 MHz, DMSO-d₆) δ 7.99 (d, J=2.4 Hz, 1H), 7.85 (d, J=8.8 Hz, 1H), 7.53 (dd, J=2.4, 8.2 Hz, 1H), 3.62 (t, J=7.2 Hz, 2H), 1.50-1.33 (m, 11H), 0.82 (t, J=7.2 Hz, 3H); ESIMS m/z 303 ([M-(C(CH₃)₃)+H]⁺).

### Example 79: Preparation of tert-butyl (4-bromo-3-nitrophenyl)carbamate (C315)

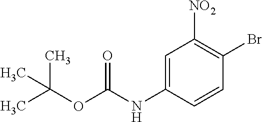 [SMILES: CC(C)(C)OC(=O)Nc1ccc(Br)c([N+](=O)[O-])c1]

To a solution of 4-bromo-3-nitroaniline (25 g, 115.2 mmol) in DCM (350 mL) were added N,N-diisopropylethylamine (51 mL, 288 mmol), N,N-dimethylpyridin-4-amine (1.4 g, 11.5 mmol), and di-tert-butyl dicarbonate (25 g, 115 mmol). The reaction mixture was stirred at room temperature for 16 h. The reaction mixture was poured into water, the organic layer was separated, and the aqueous layer was extracted with DCM (2×500 mL). The organic layer was dried over anhydrous Na₂SO₄, filtered, and concentrated under reduced pressure. The residue was triturated with n-pentane. The title compound was isolated as a pale yellow solid (26.0 g, 71%): ¹H NMR (300 MHz, CDCl₃) δ 8.04 (d, J=2.4 Hz, 1H), 7.60 (d, J=8.8 Hz, 1H), 7.60 (dd, J=2.4, 8.8 Hz, 1H), 6.68 (br s, 1H), 1.49 (s, 9H); ESIMS m/z 315 ([M−H]⁻).

### Example 80: Preparation of 4-fluoro-N,N-dimethyl-3-nitroaniline (C316)

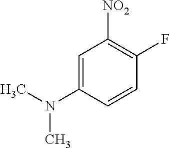

To a mixture of NaH (60% suspension in mineral oil; 1.5 g, 64.1 mmol) in DMF (150 mL) was added 4-fluoro-3-nitroaniline (5 g, 32.05 mmol) drop-wise at 0° C., and the reaction mixture was stirred at 0° C. for 30 min. Iodomethane (9.1 g, 64.1 mmol) was added drop-wise and the reaction mixture was stirred at room temperature for 16 h. The reaction mixture was poured into ice water (100 mL) and was extracted with EtOAc (2×150 mL). The organic layer was washed with water and brine, dried over anhydrous Na₂SO₄, filtered, and concentrated under reduced pressure. Purification by column chromatography (silica gel 100-200 mesh) eluting with 20-40% EtOAc-petroleum ether afforded the title compound as a brown liquid (3.5 g, 58%): ¹H NMR (400 MHz, CDCl₃) δ 7.25-7.22 (m, 1H), 7.14-7.09 (m, 1H), 6.90-6.86 (m, 1H), 2.98 (s, 6H); ESIMS m/z 185 ([M+H]⁺).

### Example 81: Preparation of 1-(1-bromoethyl)-2-nitrobenzene (C317)

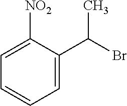

To a magnetically stirred solution of 1-ethyl-2-nitrobenzene (20 g, 132 mmol) and N-bromosuccinimide (25.9 g, 146 mmol) in dry carbon tetrachloride (441 mL) was added benzoyl peroxide (3.20 g, 13.2 mmol) in a dry 1-L round-bottomed flask under a nitrogen atmosphere. The reaction mixture was stirred at reflux for 18 h. The reaction mixture was cooled, was washed with saturated sodium bicarbonate, and concentrated. The title compound was isolated as a brown oil which was used with no additional purification (29 g, 86%): ¹H NMR (400 MHz, CDCl₃) δ 7.89 (dd, J=7.9, 1.4 Hz, 1H), 7.83 (dd, J=8.2, 1.4 Hz, 1H), 7.68-7.60 (m, 1H), 7.43 (ddd, J=8.1, 7.4, 1.4 Hz, 1H), 5.81 (q, J=6.8 Hz, 1H), 2.09 (d, J=6.8 Hz, 3H).

The following compounds were prepared in accordance to the procedure in Example 18:

### 4-(3-Bromo-1H-1,2,4-triazol-1-yl)-2-(2,2,2-trifluoroethoxy)pyridine (C318)

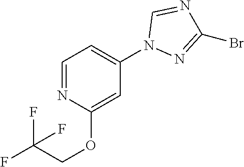 [SMILES: FC(F)(F)COc1cc(-n2cnc(Br)n2)ccn1]

The title compound was prepared and was isolated as a white solid (39 mg, 10%): ¹H NMR (400 MHz, CDCl₃) δ 8.55 (s, 1H), 8.28 (d, J=5.5 Hz, 1H), 7.32 (dd, J=5.7, 1.9 Hz, 1H), 7.19 (d, J=1.9 Hz, 1H), 4.83 (q, J=8.4 Hz, 2H); ¹⁹F NMR (376 MHz, CDCl₃) δ −73.83; EIMS m/z 322.

### 4-(3-Bromo-1H-1,2,4-triazol-1-yl)-1-(2,2,2-trifluoroethyl)pyridin-2(1H)-one (C319)

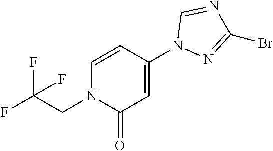 [SMILES: O=c1cc(-n2cnc(Br)n2)ccn1CC(F)(F)F]

The title compound was prepared and was isolated as a white solid (64 mg, 17%): ¹H NMR (400 MHz, CDCl₃) δ 8.50 (s, 1H), 7.46 (d, J=7.6 Hz, 1H), 6.86 (d, J=2.5 Hz, 1H), 6.75 (dd, J=7.6, 2.5 Hz, 1H), 4.65 (q, J=8.4 Hz, 2H); ¹⁹F NMR (376 MHz, CDCl₃) δ −70.58; ESIMS m/z 323 ([M+H]⁺).

### 5-(3-Bromo-1H-1,2,4-triazol-1-yl)-2-(2,2,2-trifluoroethoxy)pyridine (C320)

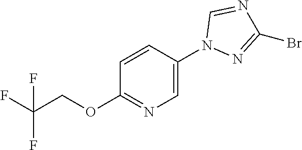 [SMILES: FC(F)(F)COc1ccc(-n2cnc(Br)n2)cn1]

The title compound was prepared and was isolated as a colorless oil (56 mg, 2%): ¹H NMR (400 MHz, CDCl₃) δ 8.44 (dd, J=2.8, 0.7 Hz, 1H), 8.37 (s, 1H), 7.94 (dd, J=8.9, 2.8 Hz, 1H), 7.03 (dd, J=8.9, 0.7 Hz, 1H), 4.82 (q, J=8.4 Hz, 2H); ¹⁹F NMR (376 MHz, CDCl₃) δ −73.76; ESIMS m/z 323 ([M+H]⁺).

### 3-Bromo-1-(4-ethoxyphenyl)-1H-1,2,4-triazole (C321)

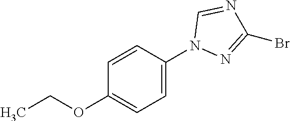

The title compound was prepared and was isolated as a white solid (592 mg, 21%): ¹H NMR (400 MHz, CDCl₃) δ 8.31 (s, 1H), 7.59-7.44 (m, 2H), 7.05-6.94 (m, 2H), 4.08 (q, J=7.0 Hz, 2H), 1.45 (t, J=7.0 Hz, 3H); ESIMS m/z 268.

The following compounds were prepared in accordance to the procedure in Example 20:

### 2-Fluoro-4-(1-(4-(pentafluoro-λ⁶-sulfaneyl)phenyl)-1H-1,2,4-triazol-3-yl)aniline (C322)

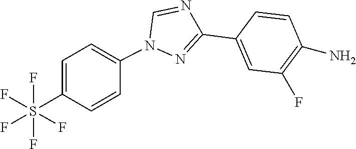 [SMILES: Nc1ccc(-c2ncn(-c3ccc(S(F)(F)(F)(F)F)cc3)n2)cc1F]

The title compound was prepared and was isolated as an off-white solid (1.3 g, 61%): ¹H NMR (400 MHz, CDCl₃) δ 8.60 (s, 1H), 7.99-7.75 (m, 6H), 6.95-6.78 (m, 1H), 3.95 (s, 2H); ¹⁹F NMR (376 MHz, CDCl₃) δ −135.26.

### 2-Methyl-4-(1-(4-(trifluoromethoxy)phenyl)-1H-1,2,4-triazol-3-yl)aniline (C323)

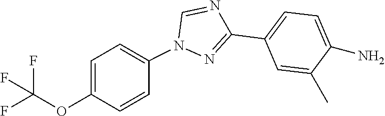 [SMILES: Cc1cc(-c2ncn(-c3ccc(OC(F)(F)F)cc3)n2)ccc1N]

The title compound was prepared and was isolated as a yellow solid (1.1 g, 48%): ¹H NMR (400 MHz, CDCl₃) δ 8.51 (s, 1H), 7.95-7.83 (m, 2H), 7.83-7.75 (m, 2H), 7.41-7.32 (m, 2H), 6.75 (d, J=8.1 Hz, 1H), 3.81 (s, 2H), 2.25 (s, 3H); ¹⁹F NMR (376 MHz, CDCl₃) δ −57.66-−58.23 (m), −58.49; ESIMS m/z 335 ([M+H]⁺).

### 2-Methyl-4-(1-(4-(pentafluoro-λ⁶-sulfaneyl)phenyl)-1H-1,2,4-triazol-3-yl)aniline (C324)

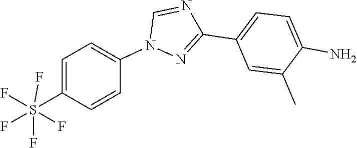 [SMILES: Cc1cc(-c2ncn(-c3ccc(S(F)(F)(F)(F)F)cc3)n2)ccc1N]

The title compound was prepared and was isolated as an off-white solid (1.29 g, 57%): ¹H NMR (400 MHz, CDCl₃) δ 8.60 (s, 1H), 7.99-7.81 (m, 6H), 6.76 (d, J=8.2 Hz, 1H), 3.84 (s, 2H), 2.25 (s, 3H); ESIMS m/z 377 ([M+H]⁺).

### 2-Fluoro-4-(1-(4-(perfluoroethoxy)phenyl)-1H-1,2,4-triazol-3-yl)aniline (C325)

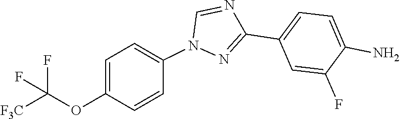 [SMILES: Nc1ccc(-c2ncn(-c3ccc(OC(F)(F)C(F)(F)F)cc3)n2)cc1F]

The title compound was prepared and was isolated as a white solid (1.3 g, 58%): ¹H NMR (400 MHz, CDCl₃) δ 8.52 (s, 1H), 7.91-7.74 (m, 4H), 7.38 (d, J=8.5 Hz, 2H), 6.85 (t, J=8.6 Hz, 1H), 3.93 (s, 2H); ¹⁹F NMR (376 MHz, CDCl₃) δ −85.88, −88.35, −135.21; ESIMS m/z 389 ([M+H]⁺).

### 2-Methyl-4-(1-(4-(perfluoroethoxy)phenyl)-1H-1,2,4-triazol-3-yl)aniline (C326)

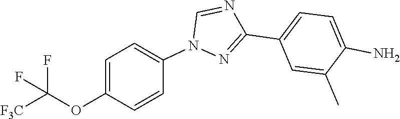 [SMILES: Cc1cc(-c2ncn(-c3ccc(OC(F)(F)C(F)(F)F)cc3)n2)ccc1N]

The title compound was prepared and was isolated as a white solid (1.25 g, 55%): ¹H NMR (400 MHz, CDCl₃) δ 8.51 (d, J=1.5 Hz, 1H), 7.95-7.73 (m, 4H), 7.37 (d, J=8.4 Hz, 2H), 6.75 (d, J=8.1 Hz, 1H), 3.81 (s, 2H), 2.25 (s, 3H); ¹⁹F NMR (376 MHz, CDCl₃) δ −85.90, −87.85; ESIMS m/z 385 ([M+H]⁺). 4-(1-(4-(Difluoromethyl)phenyl)-1H-1,2,4-triazol-3-yl)-2-methylaniline (C327)

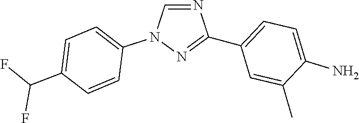 [SMILES: Cc1cc(-c2ncn(-c3ccc(C(F)F)cc3)n2)ccc1N]

The title compound was prepared and was isolated as a white solid (0.43 g, 53%): ¹H NMR (500 MHz, CDCl₃) δ 8.57 (s, 1H), 7.95-7.80 (m, 4H), 7.66 (dd, J=8.6, 1.3 Hz, 2H), 6.88-6.57 (m, 2H), 3.81 (s, 2H), 2.25 (s, 3H); ¹⁹F NMR (471 MHz, CDCl₃) δ −110.67; ESIMS m/z 301 ([M+H]⁺).

### 4-(1-(4-(Difluoromethyl)phenyl)-1H-1,2,4-triazol-3-yl)-2-fluoroaniline (C328)

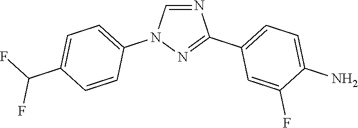 [SMILES: Nc1ccc(-c2ncn(-c3ccc(C(F)F)cc3)n2)cc1F]

The title compound was prepared and was isolated as an off-white solid (0.39 g, 47%): ¹H NMR (400 MHz, CDCl₃) δ 8.58 (s, 1H), 7.94-7.76 (m, 4H), 7.67 (d, J=8.3 Hz, 2H), 7.39 (dd, J=13.0, 5.9 Hz, 1H), 6.91-6.80 (m, 1H), 6.64 (d, J=56.3 Hz, 1H), 3.93 (s, 2H); ¹⁹F NMR (376 MHz, CDCl₃) δ −110.94, −135.35; ESIMS m/z 305 ([M+H]⁺).

### 4-(1-(3,5-Bis(trifluoromethyl)phenyl)-1H-1,2,4-triazol-3-yl)-2-fluoroaniline (C329)

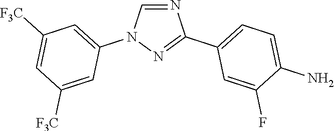 [SMILES: Nc1ccc(-c2ncn(-c3cc(C(F)(F)F)cc(C(F)(F)F)c3)n2)cc1F]

The title compound was prepared and was isolated as an off-white solid (0.45 g, 39%): ¹H NMR (400 MHz, CDCl₃) δ 8.66 (s, 1H), 8.26-8.18 (m, 2H), 7.93-7.77 (m, 3H), 6.91-6.78 (m, 1H), 3.97 (s, 2H); ¹⁹F NMR (376 MHz, CDCl₃) δ −63.00, −135.23; ESIMS m/z 391 ([M+H]⁺).

### 4-(1-(3,5-Bis(trifluoromethyl)phenyl)-1H-1,2,4-triazol-3-yl)-2-methylaniline (C330)

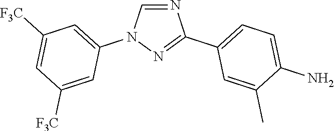 [SMILES: Cc1cc(-c2ncn(-c3cc(C(F)(F)F)cc(C(F)(F)F)c3)n2)ccc1N]

The title compound was prepared and was isolated as a light pink solid (0.72 g, 66%): ¹H NMR (400 MHz, CDCl₃) δ 8.65 (s, 1H), 8.23 (d, J=1.6 Hz, 2H), 7.96-7.82 (m, 3H), 6.76 (d, J=8.2 Hz, 1H), 3.85 (s, 2H), 2.26 (s, 3H); ¹⁹F NMR (376 MHz, CDCl₃) δ −62.98; ESIMS m/z 387 ([M+H]⁺).

### 2-Fluoro-4-(1-phenyl-1H-1,2,4-triazol-3-yl)aniline (C331)

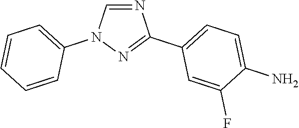 [SMILES: Nc1ccc(-c2ncn(-c3ccccc3)n2)cc1F]

The title compound was prepared and was isolated as a white crystalline solid (0.49 g, 42%): ¹H NMR (400 MHz, CDCl₃) δ 8.53 (s, 1H), 7.87-7.77 (m, 2H), 7.77-7.68 (m, 2H), 7.56-7.47 (m, 2H), 7.43-7.35 (m, 1H), 6.85 (dd, J=9.0, 8.1 Hz, 1H), 3.91 (s, 2H); ¹⁹F NMR (376 MHz, CDCl₃) δ −135.42; ESIMS m/z 254 ([M+H]⁺).

### 2-Fluoro-4-(1-(4-fluorophenyl)-1H-1,2,4-triazol-3-yl)aniline (C332)

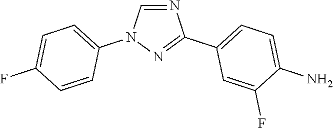 [SMILES: Nc1ccc(-c2ncn(-c3ccc(F)cc3)n2)cc1F]

The title compound was prepared and was isolated as an off-white solid (0.57 g, 48%): ¹H NMR (400 MHz, CDCl₃) δ 8.46 (s, 1H), 7.85-7.75 (m, 2H), 7.74-7.66 (m, 2H), 7.37 (dt, J=4.2, 2.4 Hz, 1H), 7.25-7.17 (m, 1H), 6.85 (dd, J=9.0, 8.1 Hz, 1H), 3.91 (s, 2H); ¹⁹F NMR (376 MHz, CDCl₃) δ −113.34, −135.38; ESIMS m/z 273 ([M+H]⁺).

### 2-Methyl-4-(1-phenyl-1H-1,2,4-triazol-3-yl)aniline (C333)

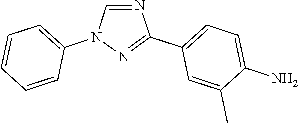 [SMILES: Cc1cc(-c2ncn(-c3ccccc3)n2)ccc1N]

The title compound was prepared and was isolated as a light brown solid (0.33 g, 56%): ¹H NMR (400 MHz, CDCl₃) δ 8.52 (s, 1H), 7.96-7.83 (m, 2H), 7.77-7.68 (m, 2H), 7.55-7.46 (m, 2H), 7.42-7.33 (m, 1H), 6.75 (d, J=8.2 Hz, 1H), 3.80 (s, 2H), 2.24 (s, 3H); ESIMS m/z 251 ([M+H]⁺).

### 4-(1-(4-Fluorophenyl)-1H-1,2,4-triazol-3-yl)-2-methylaniline (C334)

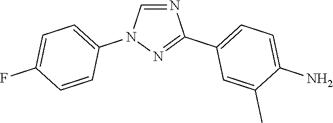 [SMILES: Cc1cc(-c2ncn(-c3ccc(F)cc3)n2)ccc1N]

The title compound was prepared and was isolated as a gray solid (0.35 g, 60%): ¹H NMR (400 MHz, CDCl₃) δ 8.45 (s, 1H), 7.95-7.82 (m, 2H), 7.74-7.64 (m, 2H), 7.24-7.16 (m, 2H), 6.75 (d, J=8.2 Hz, 1H), 3.80 (s, 2H), 2.24 (s, 3H); ¹⁹F NMR (376 MHz, CDCl₃) δ −113.64; ESIMS m/z 267 ([M+H]⁺).

### 2-Fluoro-4-(1-(4-(perfluoroethyl)phenyl)-1H-1,2,4-triazol-3-yl)aniline (C335)

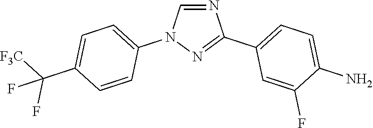 [SMILES: Nc1ccc(-c2ncn(-c3ccc(C(F)(F)C(F)(F)F)cc3)n2)cc1F]

The title compound was prepared and was isolated as a white solid (0.42 g, 73%): ¹H NMR (400 MHz, CDCl₃) δ 8.61 (s, 1H), 7.96-7.70 (m, 6H), 6.86 (dd, J=8.9, 8.2 Hz, 1H), 3.95 (s, 2H); ¹⁹F NMR (376 MHz, CDCl₃) δ −84.71, −114.86, −135.28; ESIMS m/z 373 ([M+H]⁺).

### 2-Methyl-4-(1-(4-(perfluoroethyl)phenyl)-1H-1,2,4-triazol-3-yl)aniline (C336)

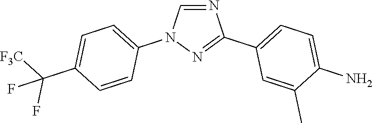 [SMILES: Cc1cc(-c2ncn(-c3ccc(C(F)(F)C(F)(F)F)cc3)n2)ccc1N]

The title compound was prepared and was isolated as a white solid (0.34 g, 60%); ¹H NMR (400 MHz, CDCl₃) δ 8.61 (s, 1H), 7.96-7.83 (m, 4H), 7.75 (d, J=8.5 Hz, 2H), 6.76 (d, J=8.1 Hz, 1H), 3.83 (s, 2H), 2.26 (s, 3H); ¹⁹F NMR (376 MHz, CDCl₃) δ −84.72, −114.84; ESIMS m/z 369 ([M+H]⁺).

### 4-(1-(5-(Trifluoromethyl)pyridin-2-yl)-1H-1,2,4-triazol-3-yl)aniline (C337)

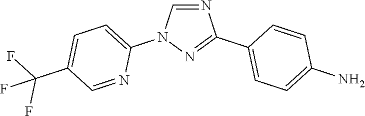 [SMILES: Nc1ccc(-c2ncn(-c3ccc(C(F)(F)F)cn3)n2)cc1]

The title compound was prepared and isolated as an off-white solid (436 mg, 80%): ¹H NMR (400 MHz, CDCl₃) δ 9.18 (s, 1H), 8.72 (dt, J=2.1, 1.0 Hz, 1H), 8.19-8.07 (m, 2H), 8.07-7.97 (m, 2H), 6.83-6.68 (m, 2H), 3.89 (s, 2H); ¹⁹F NMR (376 MHz, CDCl₃) δ −62.06; ESIMS m/z 305.

### 4-(1-(5-(Difluoromethyl)pyridin-2-yl)-1H-1,2,4-triazol-3-yl)-2-fluoroaniline (C338)

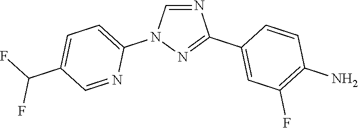 [SMILES: Nc1ccc(-c2ncn(-c3ccc(C(F)F)cn3)n2)cc1F]

The title compound was prepared and isolated as a tan solid (873 mg, 77%): ¹H NMR (400 MHz, CDCl₃) δ 9.18 (s, 1H), 8.59 (p, J=1.5 Hz, 1H), 8.12-7.99 (m, 2H), 7.90-7.78 (m, 2H), 6.97-6.81 (m, 1H), 6.70 (d, J=55.7 Hz, 1H), 3.96 (s, 2H); ¹⁹F NMR (376 MHz, CDCl₃) δ −111.92, −135.33; ESIMS m/z 305.

### 4-(1-(4-Ethoxyphenyl)-1H-1,2,4-triazol-3-yl)-2-fluoroaniline (C339)

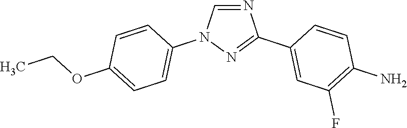 [SMILES: CCOc1ccc(-n2cnc(-c3ccc(N)c(F)c3)n2)cc1]

The title compound was prepared and isolated as an off-white solid (0.90 g, 45%): ¹H NMR (400 MHz, CDCl₃) δ 8.41 (s, 1H), 7.89-7.73 (m, 2H), 7.65-7.55 (m, 2H), 7.05-6.93 (m, 2H), 6.89-6.77 (m, 1H), 4.08 (q, J=7.0 Hz, 2H), 3.89 (s, 2H), 1.45 (t, J=7.0 Hz, 3H); ¹⁹F NMR (376 MHz, CDCl₃) δ −135.49.

The following compounds were prepared in accordance to the procedure in Example 22:

### 2-Amino-5-(1-(4-(pentafluoro-λ⁶-sulfaneyl)phenyl)-1H-1,2,4-triazol-3-yl)benzonitrile (C340)

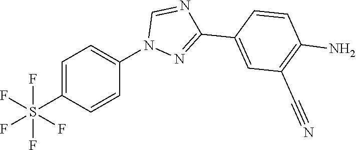 [SMILES: N#Cc1cc(-c2ncn(-c3ccc(S(F)(F)(F)(F)F)cc3)n2)ccc1N]

The title compound was prepared and was isolated as a light brown solid (729 mg, 66%): ¹H NMR (400 MHz, DMSO-d₆) δ 9.48 (s, 1H), 8.15 (d, J=1.4 Hz, 4H), 8.07-7.98 (m, 2H), 6.92 (d, J=8.7 Hz, 1H), 6.53 (s, 2H); ¹³C NMR (101 MHz, DMSO) δ 161.95, 153.14, 144.61, 139.54, 132.29, 130.94, 128.22, 119.76, 118.25, 118.06, 116.13, 93.86; ESIMS m/z 388 ([M+H]⁺).

### 4-(1-(4-(Pentafluoro-λ⁶-sulfaneyl)phenyl)-1H-1,2,4-triazol-3-yl)-2-(trifluoromethyl)aniline (C341)

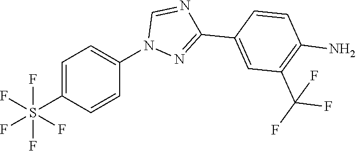 [SMILES: Nc1ccc(-c2ncn(-c3ccc(S(F)(F)(F)(F)F)cc3)n2)cc1C(F)(F)F]

The title compound was prepared and was isolated as an off-white solid (780 mg, 79%): ¹H NMR (400 MHz, CDCl₃) δ 8.61 (s, 1H), 8.29 (d, J=2.0 Hz, 1H), 8.17-8.08 (m, 1H), 7.96-7.80 (m, 4H), 6.83 (d, J=8.5 Hz, 1H), 4.40 (s, 2H); ¹⁹F NMR (376 MHz, CDCl₃) δ −62.76; ESIMS m/z 431 ([M+H]⁺).

### 2-Fluoro-4-(1-(2-(2,2,2-trifluoroethoxy)pyridin-4-yl)-1H-1,2,4-triazol-3-yl)aniline (C342)

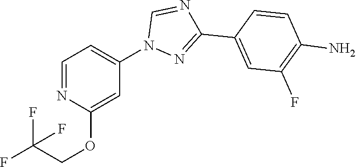 [SMILES: Nc1ccc(-c2ncn(-c3ccnc(OCC(F)(F)F)c3)n2)cc1F]

The title compound was prepared and was isolated as a white solid (24 mg, 56%): ¹H NMR (400 MHz, CDCl₃) δ 8.64 (s, 1H), 8.25 (d, J=5.7 Hz, 1H), 7.87-7.76 (m, 2H), 7.39 (dd, J=5.6, 1.9 Hz, 1H), 7.28 (d, J=1.9 Hz, 1H), 6.85 (t, J=8.6 Hz, 1H), 4.83 (q, J=8.5 Hz, 2H), 3.96 (s, 2H); ¹⁹F NMR (376 MHz, CDCl₃) δ −73.82,-135.27; ESIMS m/z 354 ([M+H]⁺).

### 4-(3-(4-Amino-3-fluorophenyl)-1H-1,2,4-triazol-1-yl)-1-(2,2,2-trifluoroethyl)pyridin-2(1H)-one (C343)

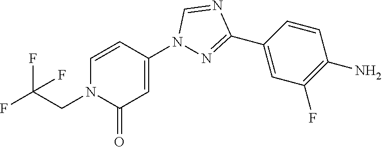 [SMILES: Nc1ccc(-c2ncn(-c3ccn(CC(F)(F)F)c(=O)c3)n2)cc1F]

The title compound was prepared and was isolated as an off-white solid (44 mg, 63%): ¹H NMR (400 MHz, DMSO-d₆) δ 9.42 (s, 1H), 7.94 (d, J=7.5 Hz, 1H), 7.68-7.60 (m, 2H), 7.07-6.98 (m, 2H), 6.85 (t, J=8.9 Hz, 1H), 5.66 (s, 2H), 4.91 (q, J=9.2 Hz, 2H); ¹⁹F NMR (376 MHz, DMSO-d₆) δ−69.42, −134.99; ESIMS m/z 354 ([M+H]⁺).

### 2-Fluoro-4-(1-(6-(2,2,2-trifluoroethoxy)pyridin-3-yl)-1H-1,2,4-triazol-3-yl)aniline (C344)

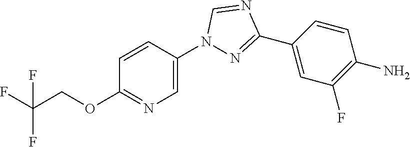 [SMILES: Nc1ccc(-c2ncn(-c3ccc(OCC(F)(F)F)nc3)n2)cc1F]

The title compound was prepared and was isolated as a light brown solid (58 mg, 95%): ¹H NMR (400 MHz, CDCl₃) δ 8.51 (dd, J=2.7, 0.7 Hz, 1H), 8.45 (s, 1H), 8.02 (dd, J=8.8, 2.8 Hz, 1H), 7.87-7.68 (m, 2H), 7.03 (dd, J=8.9, 0.7 Hz, 1H), 6.85 (dd, J=9.0, 8.1 Hz, 1H), 4.82 (q, J=8.4 Hz, 2H), 3.93 (s, 2H); ¹⁹F NMR (376 MHz, CDCl₃) δ −73.78, −135.34; ESIMS m/z 354 ([M+H]⁺).

### 3-Methyl-5-(1-(4-(trifluoromethyl)phenyl)-1H-1,2,4-triazol-3-yl)pyridin-2-amine (C345)

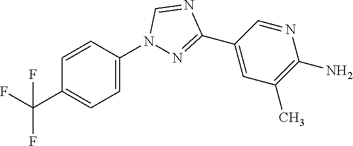 [SMILES: Cc1cc(-c2ncn(-c3ccc(C(F)(F)F)cc3)n2)cnc1N]

The title compound was prepared and was isolated as a white solid (291 mg, 53%): ¹H NMR (400 MHz, CDCl₃) δ 8.79 (d, J=2.1 Hz, 1H), 8.63 (s, 1H), 8.10-8.04 (m, 1H), 7.90 (d, J=8.4 Hz, 2H), 7.79 (d, J=8.5 Hz, 2H), 4.65 (s, 2H), 2.23 (s, 3H); ¹⁹F NMR (376 MHz, CDCl₃) δ −62.46; ESIMS m/z 320 ([M+H]⁺).

The following compounds were prepared in accordance to the procedure in Example 28:

### 1-(3,5-Bis(trifluoromethyl)phenyl)-3-bromo-1H-1,2,4-triazole (C346)

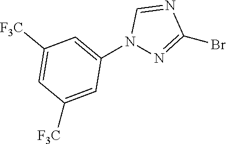 [SMILES: FC(F)(F)c1cc(-n2cnc(Br)n2)cc(C(F)(F)F)c1]

The title compound was prepared and was isolated as a white solid (3.56 g, 76%): ¹H NMR (400 MHz, CDCl₃) δ 8.59 (s, 1H), 8.16 (d, J=1.6 Hz, 2H), 7.99-7.85 (m, 1H); ¹⁹F NMR (376 MHz, CDCl₃) δ −63.05; ESIMS m/z 361 ([M+H]⁺).

### 3-Bromo-1-(4-(perfluoroethyl)phenyl)-1H-1,2,4-triazole (C347)

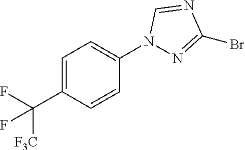 [SMILES: FC(F)(F)C(F)(F)c1ccc(-n2cnc(Br)n2)cc1]

The title compound was prepared and was isolated as a white solid (1.17 g, 24%): ¹H NMR (400 MHz, CDCl₃) δ 8.53 (s, 1H), 7.88-7.72 (m, 4H); ¹⁹F NMR (376 MHz, CDCl₃) δ −84.68, −114.97; ESIMS m/z 343 ([M+H]⁺). 3-Bromo-1-(4-(pentafluoro-λ⁶-sulfaneyl)phenyl)-1H-1,2,4-triazole (C93)

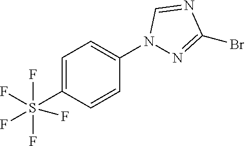 [SMILES: FS(F)(F)(F)(F)c1ccc(-n2cnc(Br)n2)cc1]

The title compound was prepared and was isolated as a white solid (2 g, 62%): ¹H NMR (400 MHz, CDCl₃) δ 8.52 (s, 1H), 7.99-7.89 (m, 2H), 7.79 (d, J=8.7 Hz, 2H); ESIMS m/z 351 ([M+H]⁺).

The following compounds were prepared in accordance to the procedure in Example 31.

### (Z)-1-(3-(5-Methyl-2-(3,3,3-trifluoropropoxy)phenyl)-4-oxothiazolidin-2-ylidene)-3-(4-(1-(4-(trifluoromethyl)phenyl)-1H-1,2,4-triazol-3-yl)phenyl)urea (J315)

The title compound was prepared and was isolated as an off-white solid (236 mg, 70%).

### (Z)-1-(3-(5-Methyl-2-(3,3,3-trifluoropropoxy)phenyl)-4-oxothiazolidin-2-ylidene)-3-(4-(1-(4-(trifluoromethoxy)phenyl)-1H-1,2,4-triazol-3-yl)phenyl)urea (J316)

The title compound was prepared and was isolated as a brown foam (206 mg, 66%).

### (Z)-1-(3-(5-(Dimethylamino)-2-isopropylphenyl)-4-oxothiazolidin-2-ylidene)-3-(2-fluoro-4-(1-(4-(trifluoromethoxy)phenyl)-1H-1,2,4-triazol-3-yl)phenyl)urea (J318)

The title compound was prepared and was isolated as an orange solid (106 mg, 55%).

### (Z)-1-(3-(5-(Dimethylamino)-2-isopropylphenyl)-4-oxothiazolidin-2-ylidene)-3-(4-(1-(4-(trifluoromethoxy)phenyl)-1H-1,2,4-triazol-3-yl)phenyl)urea (J319)

The title compound was prepared and was isolated as an orange solid (163 mg, 82%).

### (Z)-1-(3-(5-Methoxy-2-(3,3,3-trifluoropropoxy)phenyl)-4-oxothiazolidin-2-ylidene)-3-(4-(1-(4-(trifluoromethoxy)phenyl)-1H-1,2,4-triazol-3-yl)phenyl)urea (J320)

The title compound was prepared from and was isolated as an off-white solid (82 mg, 76%).

### (Z)-1-(3-(5-(Dimethylamino)-2-(3,3,3-trifluoropropoxy)phenyl)-4-oxothiazolidin-2-ylidene)-3-(4-(1-(4-(trifluoromethoxy)phenyl)-1H-1,2,4-triazol-3-yl)phenyl)urea (J321)

The title compound was prepared and was isolated as an off-white foam (95 mg, 86%).

### (Z)-1-(3-(5-Methyl-2-((2,2,2-trifluoroethoxy)methyl)phenyl)-4-oxothiazolidin-2-ylidene)-3-(4-(1-(4-(trifluoromethoxy)phenyl)-1H-1,2,4-triazol-3-yl)phenyl)urea (J331)

The title compound was prepared and was isolated as a yellow foam (85 mg, 80%).

### (Z)-1-(3-(5-Methyl-2-((2,2,2-trifluoroethoxy)methyl)phenyl)-4-oxothiazolidin-2-ylidene)-3-(4-(1-(4-(trifluoromethyl)phenyl)-1H-1,2,4-triazol-3-yl)phenyl)urea (J332)

The title compound was prepared and was isolated as a yellow foam (93 mg, 86%).

### (Z)-1-(2-Fluoro-4-(1-(4-((trifluoromethyl)thio)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J333)

The title compound was prepared and was isolated as a yellow foam (84 mg, 84%).

### (Z)-1-(3-(5-(Dimethylamino)-2-isopropylphenyl)-4-oxothiazolidin-2-ylidene)-3-(2-fluoro-4-(1-(4-((trifluoromethyl)thio)phenyl)-1H-1,2,4-triazol-3-yl)phenyl)urea (J334)

The title compound was prepared and was isolated as a white foam (56 mg, 59%).

### (Z)-1-(2-Chloro-4-(1-(4-(trifluoromethyl)phenyl)-1H-1,2,4-triazol-3-yl)phenyl)-3-(3-(2-(1-methoxyethyl)-5-methylphenyl)-4-oxothiazolidin-2-ylidene)urea (J335)

The title compound was prepared and was isolated as a white solid (23 mg, 24%).

### (Z)-1-(3-(5-(Dimethylamino)-2-isopropylphenyl)-4-oxothiazolidin-2-ylidene)-3-(2-fluoro-4-(1-(5-(trifluoromethyl)pyridin-2-yl)-1H-1,2,4-triazol-3-yl)phenyl)urea (J336)

The title compound was prepared and was isolated as an off-white solid (34 mg, 34%).

### (Z)-1-(3-(2-Isopropyl-5-methylphenyl)-4-oxothiazolidin-2-ylidene)-3-(4-(1-(5-(trifluoromethyl)pyridin-2-yl)-1H-1,2,4-triazol-3-yl)phenyl)urea (J337)

The title compound was prepared and was isolated as an off-white solid (74 mg, 74%).

### (Z)-1-(3-(2-(1-Methoxyethyl)-5-methylphenyl)-4-oxothiazolidin-2-ylidene)-3-(4-(1-(5-(trifluoromethyl)pyridin-2-yl)-1H-1,2,4-triazol-3-yl)phenyl)urea (J338)

The title compound was prepared and was isolated as an off-white solid (71 mg, 69%).

### (Z)-1-(3-(5-(Dimethylamino)-2-isopropylphenyl)-4-oxothiazolidin-2-ylidene)-3-(4-(1-(5-(trifluoromethyl)pyridin-2-yl))-1H-1,2,4-triazol-3-yl)phenyl)urea (J339)

The title compound was prepared and was isolated as an off-white solid (56 mg, 55%).

### (Z)-1-(4-(1-(5-(Difluoromethyl)pyridin-2-yl))-1H-1,2,4-triazol-3-yl)-2-fluorophenyl)-3-(3-(2-(1-methoxyethyl)-5-methylphenyl)-4-oxothiazolidin-2-ylidene)urea (J341)

The title compound was prepared and was isolated as a white solid (68 mg, 68%).

### (Z)-1-(4-(1-(5-(Difluoromethyl)pyridin-2-yl))-1H-1,2,4-triazol-3-yl)-2-fluorophenyl)-3-(3-(2-(methoxymethyl)-5-methylphenyl)-4-oxothiazolidin-2-ylidene)urea (J345)

The title compound was prepared and was isolated as an off-white solid (20 mg, 21%).

### (Z)-1-(4-(1-(5-(Difluoromethyl)pyridin-2-yl))-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J346)

The title compound was prepared and was isolated as an off-white solid (67 mg, 62%).

### (Z)-1-(4-(1-(5-(Difluoromethyl)pyridin-2-yl))-1H-1,2,4-triazol-3-yl)-2-fluorophenyl)-3-(3-(5-(dimethylamino)-2-isopropylphenyl)-4-oxothiazolidin-2-ylidene)urea (J347)

The title compound was prepared and was isolated as an off-white solid (54 mg, 53%).

### (Z)-1-(2-fluoro-4-(1-(5-(trifluoromethyl)pyridin-2-yl))-1H-pyrazol-3-yl)phenyl)-3-(3-(2-isopropyl-5-methylphenyl)-4-oxothiazolidin-2-ylidene)urea (J348)

The title compound was prepared and was isolated as an off-white solid (39 mg, 42%).

### (Z)-1-(3-(5-(Dimethylamino)-2-isopropylphenyl)-4-oxothiazolidin-2-ylidene)-3-(2-fluoro-4-(1-(5-(trifluoromethyl)pyridin-2-yl))-1H-pyrazol-3-yl)phenyl)urea (J349)

The title compound was prepared and was isolated as an off-white solid (68 mg, 69%).

### (Z)-1-(4-(1-(5-(Difluoromethyl)pyridin-2-yl))-1H-1,2,4-triazol-3-yl)-2-fluorophenyl)-3-(3-(5-(dimethylamino)-4-fluoro-2-isopropylphenyl)-4-oxothiazolidin-2-ylidene)urea (J351)

The title compound was prepared and was isolated as an off-white solid (64 mg, 61%).

### (Z)-1-(3-(5-(Dimethylamino)-4-fluoro-2-isopropylphenyl)-4-oxothiazolidin-2-ylidene)-3-(2-fluoro-4-(1-(5-(trifluoromethyl)pyridin-2-yl))-1H-1,2,4-triazol-3-yl)phenyl)urea (J352)

The title compound was prepared and was isolated as an off-white solid (63 mg, 62%).

### (Z)-1-(4-(1-(5-(Difluoromethyl)pyridin-2-yl))-1H-1,2,4-triazol-3-yl)-2-fluorophenyl)-3-(3-(5-methyl-2-(2,2,2-trifluoro-1-methoxyethyl)phenyl)-4-oxothiazolidin-2-ylidene)urea (J353)

The title compound was prepared and was isolated as a yellow solid (58 mg, 53%).

### (Z)-1-(4-(1-(5-(Difluoromethyl)pyridin-2-yl))-1H-1,2,4-triazol-3-yl)-2-fluorophenyl)-3-(3-(2-(1-ethoxyethyl)-5-methylphenyl)-4-oxothiazolidin-2-ylidene)urea (J354)

The title compound was prepared and was isolated as a yellow solid (58 mg, 57%).

### (Z)-1-(4-(1-(5-(Difluoromethyl)pyridin-2-yl))-1H-1,2,4-triazol-3-yl)-2-fluorophenyl)-3-(3-(2-isopropyl-5-methoxyphenyl)-4-oxothiazolidin-2-ylidene)urea (J355)

The title compound was prepared and was isolated as a white solid (38 mg, 38%).

### (Z)-1-(2-Fluoro-4-(1-(5-(trifluoromethyl)pyridin-2-yl))-1H-1,2,4-triazol-3-yl)phenyl)-3-(3-(5-methyl-2-(2,2,2-trifluoro-1-methoxyethyl)phenyl)-4-oxothiazolidin-2-ylidene)urea (J377)

The title compound was prepared and was isolated as an off-white solid (50 mg, 48%).

### (Z)-1-(3-(2-(1-Ethoxyethyl)-5-methylphenyl)-4-oxothiazolidin-2-ylidene)-3-(2-fluoro-4-(1-(5-(trifluoromethyl)pyridin-2-yl))-1H-1,2,4-triazol-3-yl)phenyl)urea (J378)

The title compound was prepared and was isolated as an off-white solid (52 mg, 53%).

### (Z)-1-(2-Fluoro-4-(1-(6-(trifluoromethyl)pyridazin-3-yl))-1H-pyrazol-3-yl)phenyl)-3-(3-(2-(1-methoxyethyl)-5-methylphenyl)-4-oxothiazolidin-2-ylidene)urea (J381)

The title compound was prepared and was isolated as an off-white solid (17 mg, 18%).

### (Z)-1-(2-Fluoro-4-(1-(6-(trifluoromethyl)pyridazin-3-yl))-1H-pyrazol-3-yl)phenyl)-3-(3-(5-methyl-2-((2,2,2-trifluoroethoxy)methyl)phenyl)-4-oxothiazolidin-2-ylidene)urea (J390)

The title compound was prepared and was isolated as an off-white solid (73 mg, 69%).

### (Z)-1-(3-(5-(Dimethylamino)-2-isopropylphenyl)-4-oxothiazolidin-2-ylidene)-3-(2-fluoro-4-(1-(6-(trifluoromethyl)pyridazin-3-yl))-1H-pyrazol-3-yl)phenyl)urea (J391)

The title compound was prepared and was isolated as an off-white solid (97 mg, 95%).

### (Z)-1-(3-(5-(Dimethylamino)-2-isopropylphenyl)-4-oxothiazolidin-2-ylidene)-3-(2-fluoro-4-(1-(6-(trifluoromethyl)pyridazin-3-yl))-1H-1,2,4-triazol-3-yl)phenyl)urea (J393)

The title compound was prepared and was isolated as an off-white solid (60 mg, 61%).

### (Z)-1-(2-Fluoro-4-(1-(6-(trifluoromethyl)pyridazin-3-yl))-1H-1,2,4-triazol-3-yl)phenyl)-3-(3-(5-methyl-2-((2,2,2-trifluoroethoxy)methyl)phenyl)-4-oxothiazolidin-2-ylidene)urea (J394)

The title compound was prepared and was isolated as an off-white solid (54 mg, 51%).

### (Z)-1-(3-(2-((2,2-Difluoroethoxy)methyl)-5-methylphenyl)-4-oxothiazolidin-2-ylidene)-3-(2-fluoro-4-(1-(5-(trifluoromethyl)pyridin-2-yl))-1H-1,2,4-triazol-3-yl)phenyl)urea (J400)

The title compound was prepared and was isolated as a white solid (65 mg, 63%).

### (Z)-1-(3-(2-((2,2-Difluoroethoxy)methyl)-5-methylphenyl)-4-oxothiazolidin-2-ylidene)-3-(2-fluoro-4-(1-(4-(trifluoromethoxy)phenyl))-1H-1,2,4-triazol-3-yl)phenyl)urea (J401)

The title compound was prepared and was isolated as a white solid (50 mg, 50%).

### (Z)-1-(3-(2-((2,2-Difluoroethoxy)methyl)-5-methylphenyl)-4-oxothiazolidin-2-ylidene)-3-(2-methyl-4-(1-(4-(trifluoromethoxy)phenyl))-1H-1,2,4-triazol-3-yl)phenyl)urea (J420)

The title compound was prepared and was isolated as an off-white solid (68 mg, 67%).

### (Z)-1-(2-Fluoro-4-(1-(4-(trifluoromethoxy)phenyl))-1H-1,2,4-triazol-3-yl)phenyl)-3-(3-(2-(1-methoxypropyl)-5-methylphenyl)-4-oxothiazolidin-2-ylidene)urea (J421)

The title compound was prepared and was isolated as a white foam (64 mg, 66%).

### (Z)-1-(2-Fluoro-4-(1-(4-(pentafluoro-λ⁶-sulfaneyl)phenyl))-1H-1,2,4-triazol-3-yl)phenyl)-3-(3-(5-methyl-2-((2,2,2-trifluoroethoxy)methyl)phenyl)-4-oxothiazolidin-2-ylidene)urea (J422)

The title compound was prepared and was isolated as a white foam (73 mg, 75%).

### (Z)-1-(2-Fluoro-4-(1-(4-(pentafluoro-λ⁶-sulfaneyl)phenyl))-1H-1,2,4-triazol-3-yl)phenyl)-3-(3-(2-(1-methoxyethyl)-5-methylphenyl)-4-oxothiazolidin-2-ylidene)urea (J423)

The title compound was prepared and was isolated as a white foam (51 mg, 57%).

### (Z)-1-(3-(2-((2-Chloro-2,2-difluoroethoxy)methyl)-5-methylphenyl)-4-oxothiazolidin-2-ylidene)-3-(2-methyl-4-(1-(4-(trifluoromethoxy)phenyl))-1H-1,2,4-triazol-3-yl)phenyl)urea (J436)

The title compound was prepared and was isolated as a white solid (58 mg, 55%).

### (Z)-1-(3-(2-((2-Chloro-2,2-difluoroethoxy)methyl)-5-methylphenyl)-4-oxothiazolidin-2-ylidene)-3-(2-fluoro-4-(1-(4-(trifluoromethoxy)phenyl))-1H-1,2,4-triazol-3-yl)phenyl)urea (J437)

The title compound was prepared and was isolated as a white foam (69 mg, 65%).

### (Z)-1-(2-Fluoro-4-(1-(4-(trifluoromethoxy)phenyl))-1H-1,2,4-triazol-3-yl)phenyl)-3-(3-(4-methyl-2-((2,2,2-trifluoroethoxy)methyl)phenyl)-4-oxothiazolidin-2-ylidene)urea (J438)

The title compound was prepared and was isolated as a white foam (87 mg, 85%).

### (Z)-1-(3-(2-(Ethoxymethyl)-5-methylphenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J445)

The title compound was prepared and was isolated as a yellow foam (48 mg, 52%).

### (Z)-1-(3-(2-((2,2-Difluoroethoxy)methyl)-5-methylphenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J446)

The title compound was prepared and was isolated as a white foam (70 mg, 74%).

### (Z)-1-(2-Fluoro-4-(1-(4-(pentafluoro-λ⁶-sulfaneyl)phenyl))-1H-1,2,4-triazol-3-yl)phenyl)-3-(3-(5-methyl-2-(3,3,3-trifluoropropoxy)phenyl)-4-oxothiazolidin-2-ylidene)urea (J447)

The title compound was prepared and was isolated as a white foam (47 mg, 48%).

### (Z)-1-(3-(4-Methyl-2-((2,2,2-trifluoroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J448)

The title compound was prepared and was isolated as an off-white solid (79 mg, 76%).

### (Z)-1-(3-(2-((2-Chloro-2,2-difluoroethoxy)methyl)-5-methylphenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J449)

The title compound was prepared and was isolated as a white solid (50 mg, 50%).

### (Z)-1-(3-(5-(Dimethylamino)-2-isopropylphenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J450)

The title compound was prepared and was isolated as a yellow solid (68 mg, 74%).

### (Z)-1-(2-fluoro-4-(1-(5-(trifluoromethyl)pyridin-2-yl))-1H-1,2,4-triazol-3-yl)phenyl)-3-(3-(4-methyl-2-((2,2,2-trifluoroethoxy)methyl)phenyl)-4-oxothiazolidin-2-ylidene)urea (J451)

The title compound was prepared and was isolated as a white solid (63 mg, 60%).

### (Z)-1-(2-Fluoro-4-(1-(4-(trifluoromethoxy)phenyl))-1H-1,2,4-triazol-3-yl)phenyl)-3-(3-(5-methyl-2-((2,2,2-trichloroethoxy)methyl)phenyl)-4-oxothiazolidin-2-ylidene)urea (J452)

The title compound was prepared and was isolated as a white foam (47 mg, 43%).

### (Z)-1-(2-Fluoro-4-(1-(4-(pentafluoro-λ⁶-sulfaneyl)phenyl))-1H-1,2,4-triazol-3-yl)phenyl)-3-(3-(5-methyl-2-((2,2,2-trichloroethoxy)methyl)phenyl)-4-oxothiazolidin-2-ylidene)urea (J453)

The title compound was prepared and was isolated as a white foam (39 mg, 38%).

### (Z)-1-(2-Fluoro-4-(1-(5-(trifluoromethyl)pyridin-2-yl))-1H-1,2,4-triazol-3-yl)phenyl)-3-(3-(5-methyl-2-propylphenyl)-4-oxothiazolidin-2-ylidene)urea (J454)

The title compound was prepared and was isolated as a white solid (40 mg, 42%).

### (Z)-1-(3-(4,5-Dimethyl-2-((2,2,2-trifluoroethoxy)methyl)phenyl)-4-oxothiazolidin-2-ylidene)-3-(2-fluoro-4-(1-(4-(trifluoromethoxy)phenyl))-1H-1,2,4-triazol-3-yl)phenyl)urea (J455)

The title compound was prepared and was isolated as a white foam (58 mg, 55%).

### (Z)-1-(3-(5-Methyl-2-((2,2,2-trichloroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J459)

The title compound was prepared and was isolated as an off-white solid (84 mg, 76%).

### (Z)-1-(3-(4,5-Dimethyl-2-((2,2,2-trifluoroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J460)

The title compound was prepared and was isolated as an off-white solid (85 mg, 80%).

### (Z)-1-(3-(4,5-Dimethyl-2-((2,2,2-trifluoroethoxy)methyl)phenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J497)

The title compound was prepared and was isolated as a white foam (45 mg, 45%).

### (Z)-1-(2-Fluoro-4-(1-(4-(pentafluoro-λ⁶-sulfaneyl)phenyl))-1H-1,2,4-triazol-3-yl)phenyl)-3-(3-(5-methyl-2-propylphenyl)-4-oxothiazolidin-2-ylidene)urea (J498)

The title compound was prepared and was isolated as an off-white foam (40 mg, 46%).

### (Z)-1-(3-(2-((2,2-Difluoropropoxy)methyl)-5-methylphenyl)-4-oxothiazolidin-2-ylidene)-3-(2-methyl-4-(1-(4-(trifluoromethoxy)phenyl))-1H-1,2,4-triazol-3-yl)phenyl)urea (J534)

The title compound was prepared and was isolated as a white solid (76 mg, 74%).

### (Z)-1-(3-(2-((2,2-Difluoropropoxy)methyl)-5-methylphenyl)-4-oxothiazolidin-2-ylidene)-3-(2-fluoro-4-(1-(4-(trifluoromethoxy)phenyl))-1H-1,2,4-triazol-3-yl)phenyl)urea (J535)

The title compound was prepared and was isolated as a white solid (20 mg, 20%).

### (Z)-1-(2-Fluoro-4-(1-(4-(pentafluoro-λ⁶-sulfaneyl)phenyl))-1H-1,2,4-triazol-3-yl)phenyl)-3-(3-(2-(methoxymethyl)-5-methylphenyl)-4-oxothiazolidin-2-ylidene)urea (J539)

The title compound was prepared and was isolated as a yellow solid (55 mg, 62%).

### (Z)-1-(3-(5-Methyl-2-((3,3,3-trifluoropropoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J540)

The title compound was prepared and was isolated as an off-white solid (71 mg, 67%).

### (Z)-1-(2-Fluoro-4-(1-(4-(pentafluoro-λ⁶-sulfaneyl)phenyl))-1H-1,2,4-triazol-3-yl)phenyl)-3-(3-(5-methyl-2-propoxyphenyl)-4-oxothiazolidin-2-ylidene)urea (J541)

The title compound was prepared and was isolated as an off-white solid (49 mg, 55%).

### (Z)-1-(2-Fluoro-4-(1-(4-(trifluoromethoxy)phenyl))-1H-1,2,4-triazol-3-yl)phenyl)-3-(3-(5-methyl-2-((3,3,3-trifluoropropoxy)methyl)phenyl)-4-oxothiazolidin-2-ylidene)urea (J543)

The title compound was prepared and was isolated as an off-white solid (58 mg, 55%).

### (Z)-1-(2-Fluoro-4-(1-(4-(pentafluoro-λ⁶-sulfaneyl)phenyl))-1H-1,2,4-triazol-3-yl)phenyl)-3-(3-(5-methyl-2-(propoxymethyl)phenyl)-4-oxothiazolidin-2-ylidene)urea (J544)

The title compound was prepared and was isolated as a white foam (51 mg, 56%).

### (Z)-1-(2-Fluoro-4-(1-(4-(trifluoromethoxy)phenyl))-1H-1,2,4-triazol-3-yl)phenyl)-3-(3-(5-methyl-2-(propoxymethyl)phenyl)-4-oxothiazolidin-2-ylidene)urea (J545)

The title compound was prepared and was isolated as a white foam (58 mg, 60%).

### (Z)-1-(3-(5-Methyl-2-(propoxymethyl)phenyl)-4-oxothiazolidin-2-ylidene)-3-(2-methyl-4-(1-(4-(trifluoromethoxy)phenyl))-1H-1,2,4-triazol-3-yl)phenyl)urea (J546)

The title compound was prepared and was isolated as an off-white solid (60 mg, 62%).

### (Z)-1-(2-Fluoro-4-(1-(4-(trifluoromethyl)phenyl))-1H-1,2,4-triazol-3-yl)phenyl)-3-(3-(5-methyl-2-((3,3,3-trifluoropropoxy)methyl)phenyl)-4-oxothiazolidin-2-ylidene)urea (J547)

The title compound was prepared and was isolated as a white solid. (48 mg, 45%).

### (Z)-1-(3-(2-(Ethoxymethyl)-5-methylphenyl)-4-oxothiazolidin-2-ylidene)-3-(2-methyl-4-(1-(4-(pentafluoro-λ⁶-sulfaneyl)phenyl))-1H-1,2,4-triazol-3-yl)phenyl)urea (J548)

The title compound was prepared and was isolated as a yellow solid (50 mg, 55%).

### (Z)-1-(3-(5-Methyl-2-((2,2,2-trifluoroethoxy)methyl)phenyl)-4-oxothiazolidin-2-ylidene)-3-(2-methyl-4-(1-(4-(pentafluoro-λ⁶-sulfaneyl)phenyl))-1H-1,2,4-triazol-3-yl)phenyl)urea (J549)

The title compound was prepared and was isolated as an off-white solid (60 mg, 61%).

### (Z)-1-(2-Fluoro-4-(1-(4-(trifluoromethyl)phenyl))-1H-1,2,4-triazol-3-yl)phenyl)-3-(3-(5-methyl-2-((2,2,2-trichloroethoxy)methyl)phenyl)-4-oxothiazolidin-2-ylidene)urea (J550)

The title compound was prepared and was isolated as a white solid (65 mg, 57%).

### (Z)-1-(2-Fluoro-4-(1-(4-(trifluoromethoxy)phenyl))-1H-1,2,4-triazol-3-yl)phenyl)-3-(3-(2-((2-fluoroethoxy)methyl)-5-methylphenyl)-4-oxothiazolidin-2-ylidene)urea (J551)

The title compound was prepared and was isolated as a white foam (62 mg, 64%).

### (Z)-1-(2-Fluoro-4-(1-(4-(pentafluoro-λ⁶-sulfaneyl)phenyl))-1H-1,2,4-triazol-3-yl)phenyl)-3-(3-(2-((2-fluoroethoxy)methyl)-5-methylphenyl)-4-oxothiazolidin-2-ylidene)urea (J552)

The title compound was prepared and was isolated as a yellow foam (58 mg, 63%).

### (Z)-1-(3-(2-((2,2-Dichloroethoxy)methyl)-5-methylphenyl)-4-oxothiazolidin-2-ylidene)-3-(2-fluoro-4-(1-(4-(trifluoromethoxy)phenyl))-1H-1,2,4-triazol-3-yl)phenyl)urea (J553)

The title compound was prepared and was isolated as a white solid (57 mg, 54%).

### (Z)-1-(3-(2-((2,2-Dichloroethoxy)methyl)-5-methylphenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J554)

The title compound was prepared and was isolated as a white solid (56 mg, 56%).

### (Z)-1-(3-(2-((2,2-Dichloroethoxy)methyl)-5-methylphenyl)-4-oxothiazolidin-2-ylidene)-3-(2-methyl-4-(1-(4-(trifluoromethoxy)phenyl))-1H-1,2,4-triazol-3-yl)phenyl)urea (J555)

The title compound was prepared and was isolated as a white solid (68 mg, 64%).

### (Z)-1-(3-(2-((2,2-Dichloroethoxy)methyl)-5-methylphenyl)-4-oxothiazolidin-2-ylidene)-3-(2-methyl-4-(1-(4-(pentafluoro-λ⁶-sulfaneyl)phenyl))-1H-1,2,4-triazol-3-yl)phenyl)urea (J556)

The title compound was prepared and was isolated as a white solid (51 mg, 51%).

### (Z)-1-(3-(2-((2-Fluoroethoxy)methyl)-5-methylphenyl)-4-oxothiazolidin-2-ylidene)-3-(2-methyl-4-(1-(4-(pentafluoro-λ⁶-sulfaneyl)phenyl))-1H-1,2,4-triazol-3-yl)phenyl)urea (J557)

The title compound was prepared and was isolated as a white foam (48 mg, 52%).

### (Z)-1-(3-(2-((2-Fluoroethoxy)methyl)-5-methylphenyl)-4-oxothiazolidin-2-ylidene)-3-(2-methyl-4-(1-(4-(trifluoromethoxy)phenyl))-1H-1,2,4-triazol-3-yl)phenyl)urea (J558)

The title compound was prepared and was isolated as a white foam (49 mg, 50%).

### (Z)-1-(3-(2-((2,2-Dichloroethoxy)methyl)-5-methylphenyl)-4-oxothiazolidin-2-ylidene)-3-(2-fluoro-4-(1-(4-(trifluoromethyl)phenyl))-1H-1,2,4-triazol-3-yl)phenyl)urea (J559)

The title compound was prepared and was isolated as a white solid (25 mg, 23%).

### (Z)-1-(3-(4,5-Dimethoxy-2-((2,2,2-trifluoroethoxy)methyl)phenyl)-4-oxothiazolidin-2-ylidene)-3-(2-fluoro-4-(1-(4-(trifluoromethoxy)phenyl))-1H-1,2,4-triazol-3-yl)phenyl)urea (J560)

The title compound was prepared and was isolated as an off-white solid (75 mg, 68%).

### (Z)-1-(3-(4,5-Dimethoxy-2-((2,2,2-trifluoroethoxy)methyl)phenyl)-4-oxothiazolidin-2-ylidene)-3-(2-fluoro-4-(1-(4-(pentafluoro-λ⁶-sulfaneyl)phenyl))-1H-1,2,4-triazol-3-yl)phenyl)urea (J561)

The title compound was prepared and was isolated as an off-white solid (53 mg, 51%).

### (Z)-1-(2-Fluoro-4-(1-(4-(trifluoromethoxy)phenyl))-1H-1,2,4-triazol-3-yl)phenyl)-3-(3-(5-methoxy-2-((2,2,2-trifluoroethoxy)methyl)phenyl)-4-oxothiazolidin-2-ylidene)urea (J564)

The title compound was prepared and was isolated as an off-white solid (54 mg, 52%).

### (Z)-1-(2-Fluoro-4-(1-(4-(pentafluoro-λ⁶-sulfaneyl)phenyl))-1H-1,2,4-triazol-3-yl)phenyl)-3-(3-(5-methoxy-2-((2,2,2-trifluoroethoxy)methyl)phenyl)-4-oxothiazolidin-2-ylidene)urea (J565)

The title compound was prepared and was isolated as a white foam (45 mg, 45%).

### (Z)-1-(2-Fluoro-4-(1-(4-(trifluoromethoxy)phenyl))-1H-1,2,4-triazol-3-yl)phenyl)-3-(3-(3-methyl-2-((2,2,2-trifluoroethoxy)methyl)phenyl)-4-oxothiazolidin-2-ylidene)urea (J566)

The title compound was prepared and was isolated as a white foam (43 mg, 42%).

### (Z)-1-(2-Fluoro-4-(1-(4-(pentafluoro-λ⁶-sulfaneyl)phenyl))-1H-1,2,4-triazol-3-yl)phenyl)-3-(3-(3-methyl-2-((2,2,2-trifluoroethoxy)methyl)phenyl)-4-oxothiazolidin-2-ylidene)urea (J567)

The title compound was prepared and was isolated as a white foam (43 mg, 44%).

### (Z)-1-(3-(5-ethoxy-2-((2,2,2-trifluoroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J575)

The title compound was prepared and was isolated as an off-white foam (72 mg, 68%).

### (Z)-1-(3-(3-Methyl-2-((2,2,2-trifluoroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J576)

The title compound was prepared and was isolated as an off-white foam (73 mg, 71%).

### (Z)-1-(3-(5-Methoxy-2-((2,2,2-trifluoroethoxy)methyl)phenyl)-4-oxothiazolidin-2-ylidene)-3-(2-methyl-4-(1-(4-(pentafluoro-λ⁶-sulfaneyl)phenyl))-1H-1,2,4-triazol-3-yl)phenyl)urea (J577)

The title compound was prepared and was isolated as an off-white solid (73 mg, 73%).

### (Z)-1-(3-(3-Methyl-2-((2,2,2-trifluoroethoxy)methyl)phenyl)-4-oxothiazolidin-2-ylidene)-3-(2-methyl-4-(1-(4-(pentafluoro-λ⁶-sulfaneyl)phenyl))-1H-1,2,4-triazol-3-yl)phenyl)urea (J578)

The title compound was prepared and was isolated as an off-white solid (65 mg, 67%).

### (Z)-1-(3-(3-Methyl-2-((2,2,2-trifluoroethoxy)methyl)phenyl)-4-oxothiazolidin-2-ylidene)-3-(2-methyl-4-(1-(4-(trifluoromethyl)phenyl))-1H-1,2,4-triazol-3-yl)phenyl)urea (J579)

The title compound was prepared and was isolated as an off-white foam (65 mg, 61%).

### (Z)-1-(3-(5-Methoxy-2-((2,2,2-trifluoroethoxy)methyl)phenyl)-4-oxothiazolidin-2-ylidene)-3-(2-methyl-4-(1-(4-(trifluoromethyl)phenyl))-1H-1,2,4-triazol-3-yl)phenyl)urea (J580)

The title compound was prepared and was isolated as an off-white foam (44 mg, 41%).

### (Z)-1-(2-Fluoro-4-(1-(4-(trifluoromethyl)phenyl))-1H-1,2,4-triazol-3-yl)phenyl)-3-(3-(5-methoxy-2-((2,2,2-trifluoroethoxy)methyl)phenyl)-4-oxothiazolidin-2-ylidene)urea (J581)

The title compound was prepared and was isolated as an off-white foam (44 mg, 41%).

### (Z)-1-(3-(4,5-Dimethyl-2-((2,2,2-trifluoroethoxy)methyl)phenyl)-4-oxothiazolidin-2-ylidene)-3-(4-(1-(4-ethoxyphenyl))-1H-1,2,4-triazol-3-yl)-2-fluorophenyl)urea (J620)

The title compound was prepared and was isolated as an off-white solid (61 mg, 54%).

### (Z)-1-(4-(1-(4-Ethoxyphenyl))-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J625)

The title compound was prepared and was isolated as a white solid (44 mg, 40%).

### (Z)-1-(3-(5-Ethyl-2-((2,2,2-trifluoroethoxy)methyl)phenyl)-4-oxothiazolidin-2-ylidene)-3-(2-fluoro-4-(1-(4-(trifluoromethoxy)phenyl))-1H-1,2,4-triazol-3-yl)phenyl)urea (J634)

The title compound was prepared and was isolated as a white solid (52 mg, 50%).

### (Z)-1-(3-(5-Ethyl-2-((2,2,2-trifluoroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J641)

The title compound was prepared and was isolated as a white solid (44 mg, 42%).

### (Z)-1-(3-(5-Ethyl-2-((2,2,2-trifluoroethoxy)methyl)phenyl)-4-oxothiazolidin-2-ylidene)-3-(2-fluoro-4-(1-(4-(trifluoromethyl)phenyl))-1H-1,2,4-triazol-3-yl)phenyl)urea (J642)

The title compound was prepared and was isolated as a white solid (44 mg, 41%).

### (Z)-1-(4-(1-(4-(Difluoromethoxy)phenyl))-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J643)

The title compound was prepared and was isolated as a white solid (26 mg, 25%).

### (Z)-1-(4-(1-(4-(difluoromethoxy)phenyl))-1H-1,2,4-triazol-3-yl)-2-fluorophenyl)-3-(3-(5-ethyl-2-((2,2,2-trifluoroethoxy)methyl)phenyl)-4-oxothiazolidin-2-ylidene)urea (J644)

The title compound was prepared and was isolated as a white solid (41 mg, 38%).

### (Z)-1-(2-Methyl-4-(1-(4-(trifluoromethoxy)phenyl)-1H-1,2,4-triazol-3-yl)phenyl)-3-(4-oxo-3-(2-((2,2,2-trifluoroethoxy)methyl)phenyl)thiazolidin-2-ylidene)urea (J653)

The title compound was prepared and was isolated as a white solid (46 mg, 44%).

### (Z)-1-(2-Fluoro-4-(1-(4-(trifluoromethoxy)phenyl)-1H-1,2,4-triazol-3-yl)phenyl)-3-(4-oxo-3-(2-((2,2,2-trifluoroethoxy)methyl)phenyl)thiazolidin-2-ylidene)urea (J659)

The title compound was prepared and was isolated as a white solid (49 mg, 47%).

### (Z)-1-(3-(4,5-Dimethyl-2-((2,2,2-trifluoroethoxy)methyl)phenyl)-4-oxothiazolidin-2-ylidene)-3-(2-fluoro-4-(1-(4-(trifluoromethyl)phenyl))-1H-1,2,4-triazol-3-yl)phenyl)urea (J660)

The title compound was prepared and was isolated as a white foam (59 mg, 55%).

### (Z)-1-(2-Fluoro-4-(1-(4-(trifluoromethyl)phenyl))-1H-1,2,4-triazol-3-yl)phenyl)-3-(4-oxo-3-(2-((2,2,2-trifluoroethoxy)methyl)phenyl)thiazolidin-2-ylidene)urea (J661)

The title compound was prepared and was isolated as a white foam (47 mg, 46%).

### (Z)-1-(2-Fluoro-4-(1-(4-(perfluoroethoxy)phenyl))-1H-1,2,4-triazol-3-yl)phenyl)-3-(3-(5-methyl-2-((2,2,2-trifluoroethoxy)methyl)phenyl)-4-oxothiazolidin-2-ylidene)urea (J662)

The title compound was prepared and was isolated as a white solid (46 mg, 48%).

### (Z)-1-(3-(2-(Ethoxymethyl)-5-methylphenyl)-4-oxothiazolidin-2-ylidene)-3-(2-fluoro-4-(1-(4-(perfluoroethoxy)phenyl)-1H-1,2,4-triazol-3-yl)phenyl)urea (J663)

The title compound was prepared and was isolated as a yellow solid (37 mg, 42%).

### (Z)-1-(2-Fluoro-4-(1-(4-(perfluoroethoxy)phenyl))-1H-1,2,4-triazol-3-yl)phenyl)-3-(3-(3-methyl-2-((2,2,2-trifluoroethoxy)methyl)phenyl)-4-oxothiazolidin-2-ylidene)urea (J665)

The title compound was prepared and was isolated as a white solid (47 mg, 49%).

### (Z)-1-(2-Fluoro-4-(1-(4-(trifluoromethyl)phenyl))-1H-1,2,4-triazol-3-yl)phenyl)-3-(3-(3-methyl-2-((2,2,2-trifluoroethoxy)methyl)phenyl)-4-oxothiazolidin-2-ylidene)urea (J677)

The title compound was prepared and was isolated as a white foam (42 mg, 40%).

### (Z)-1-(3-(2,3-Dimethyl-6-((2,2,2-trifluoroethoxy)methyl)phenyl)-4-oxothiazolidin-2-ylidene)-3-(2-fluoro-4-(1-(4-(trifluoromethoxy)phenyl))-1H-1,2,4-triazol-3-yl)phenyl)urea (J678)

The title compound was prepared and was isolated as a white solid (56 mg, 53%).

### (Z)-1-(3-(2,3-Dimethyl-6-((2,2,2-trifluoroethoxy)methyl)phenyl)-4-oxothiazolidin-2-ylidene)-3-(2-methyl-4-(1-(4-(trifluoromethoxy)phenyl))-1H-1,2,4-triazol-3-yl)phenyl)urea (J679)

The title compound was prepared and was isolated as an off-white foam (63 mg, 60%).

### (Z)-1-(3-(2,3-Dimethyl-6-((2,2,2-trifluoroethoxy)methyl)phenyl)-4-oxothiazolidin-2-ylidene)-3-(2-fluoro-4-(1-(4-(trifluoromethyl)phenyl))-1H-1,2,4-triazol-3-yl)phenyl)urea (J680)

The title compound was prepared from and was isolated as an off-white foam (50 mg, 46%).

### (Z)-1-(3-(5-Methyl-2-((2,2,2-trifluoroethoxy)methyl)phenyl)-4-oxothiazolidin-2-ylidene)-3-(2-methyl-4-(1-(4-(perfluoroethoxy)phenyl))-1H-1,2,4-triazol-3-yl)phenyl)urea (J681)

The title compound was prepared and was isolated as an off-white solid (61 mg, 63%).

### (Z)-1-(3-(3-Methyl-2-((2,2,2-trifluoroethoxy)methyl)phenyl)-4-oxothiazolidin-2-ylidene)-3-(2-methyl-4-(1-(4-(perfluoroethoxy)phenyl))-1H-1,2,4-triazol-3-yl)phenyl)urea (J682)

The title compound was prepared and was isolated as an off-white solid (67 mg, 69%).

### (Z)-1-(4-(1-(4-(Difluoromethyl)phenyl)-1H-1,2,4-triazol-3-yl)-2-methylphenyl)-3-(3-(5-methyl-2-((2,2,2-trifluoroethoxy)methyl)phenyl)-4-oxothiazolidin-2-ylidene)urea (J684)

The title compound was prepared and was isolated as a yellow solid (84 mg, 74%).

### (Z)-1-(4-(1-(4-(Difluoromethyl)phenyl)-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J686)

The title compound was prepared and was isolated as a white solid (76 mg, 68%).

### (Z)-1-(2-Fluoro-4-(1-(3-(trifluoromethoxy)phenyl))-1H-1,2,4-triazol-3-yl)phenyl)-3-(3-(5-methyl-2-((2,2,2-trifluoroethoxy)methyl)phenyl)-4-oxothiazolidin-2-ylidene)urea (J705)

The title compound was prepared and was isolated as an off-white solid (63 mg, 61%).

### (Z)-1-(2-Fluoro-4-(1-(3-(trifluoromethoxy)phenyl))-1H-1,2,4-triazol-3-yl)phenyl)-3-(3-(3-methyl-2-((2,2,2-trifluoroethoxy)methyl)phenyl)-4-oxothiazolidin-2-ylidene)urea (J706)

The title compound was prepared and was isolated as an off-white solid (60 mg, 58%).

### (Z)-1-(2-Fluoro-4-(1-(3-(trifluoromethyl)phenyl))-1H-1,2,4-triazol-3-yl)phenyl)-3-(3-(5-methyl-2-((2,2,2-trifluoroethoxy)methyl)phenyl)-4-oxothiazolidin-2-ylidene)urea (J710)

The title compound was prepared and was isolated as an off-white foam (63 mg, 60%).

### (Z)-1-(3-(2-(Ethoxymethyl)-5-methylphenyl)-4-oxothiazolidin-2-ylidene)-3-(2-fluoro-4-(1-(3-(trifluoromethoxy)phenyl))-1H-1,2,4-triazol-3-yl)phenyl)urea (J711)

The title compound was prepared and was isolated as a yellow foam (72 mg, 74%).

### (Z)-1-(3-(5-Methyl-2-((2,2,2-trifluoroethoxy)methyl)phenyl)-4-oxothiazolidin-2-ylidene)-3-(2-methyl-4-(1-(3-(trifluoromethoxy)phenyl))-1H-1,2,4-triazol-3-yl)phenyl)urea (J722)

The title compound was prepared and was isolated as a yellow solid (71 mg, 69%).

### (Z)-1-(3-(2-(Ethoxymethyl)-5-methylphenyl)-4-oxothiazolidin-2-ylidene)-3-(2-fluoro-4-(1-(3-(trifluoromethyl)phenyl))-1H-1,2,4-triazol-3-yl)phenyl)urea (J723)

The title compound was prepared and was isolated as a yellow solid (72 mg, 72%).

### (Z)-1-(3-(2-(Ethoxymethyl)-5-methylphenyl)-4-oxothiazolidin-2-ylidene)-3-(2-methyl-4-(1-(3-(trifluoromethoxy)phenyl))-1H-1,2,4-triazol-3-yl)phenyl)urea (J726)

The title compound was prepared and was isolated as an off-white solid (74 mg, 75%).

### (Z)-1-(3-(5-Methyl-2-((2,2,2-trifluoroethoxy)methyl)phenyl)-4-oxothiazolidin-2-ylidene)-3-(2-methyl-4-(1-(3-(trifluoromethyl)phenyl))-1H-1,2,4-triazol-3-yl)phenyl)urea (J727)

The title compound was prepared and was isolated as an off-white solid (75 mg, 71%).

### (Z)-1-(3-(4,5-Dimethyl-2-((2,2,2-trifluoroethoxy)methyl)phenyl)-4-oxothiazolidin-2-ylidene)-3-(2-fluoro-4-(1-(3-(trifluoromethoxy)phenyl))-1H-1,2,4-triazol-3-yl)phenyl)urea (J738)

The title compound was prepared and was isolated as a white solid (32 mg, 30%).

### (Z)-1-(2-Fluoro-4-(1-(p-tolyl))-1H-1,2,4-triazol-3-yl)phenyl)-3-(3-(5-methyl-2-((2,2,2-trifluoroethoxy)methyl)phenyl)-4-oxothiazolidin-2-ylidene)urea (J752)

The title compound was prepared and was isolated as a yellow solid (93 mg, 80%).

### (Z)-1-(3-(2-(Ethoxymethyl)-5-methylphenyl)-4-oxothiazolidin-2-ylidene)-3-(2-fluoro-4-(1-(p-tolyl)-1H-1,2,4-triazol-3-yl)phenyl)urea (J753)

The title compound was prepared and was isolated as a yellow solid (82 mg, 75%).

### (Z)-1-(2-Fluoro-4-(1-(4-methoxyphenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J754)

The title compound was prepared and was isolated as a yellow foam (53 mg, 47%).

### (Z)-1-(3-(2-(Ethoxymethyl)-5-methylphenyl)-4-oxothiazolidin-2-ylidene)-3-(2-fluoro-4-(1-(4-methoxyphenyl)-1H-1,2,4-triazol-3-yl)phenyl)urea (J755)

The title compound was prepared and was isolated as a yellow foam (71 mg, 69%).

### (Z)-1-(3-(4,5-Dimethyl-2-((2,2,2-trifluoroethoxy)methyl)phenyl)-4-oxothiazolidin-2-ylidene)-3-(2-fluoro-4-(1-(4-methoxyphenyl)-1H-1,2,4-triazol-3-yl)phenyl)urea (J756)

The title compound was prepared and was isolated as an off-white solid (67 mg, 58%).

### (Z)-1-(3-(2-((2,2-Difluoroethoxy)methyl)-5-methylphenyl)-4-oxothiazolidin-2-ylidene)-3-(2-fluoro-4-(1-(4-methoxyphenyl)-1H-1,2,4-triazol-3-yl)phenyl)urea (J757)

The title compound was prepared and was isolated as an off-white solid (65 mg, 59%).

### (Z)-1-(4-(1-(3,5-Bis(trifluoromethyl)phenyl)-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J760)

The title compound was prepared and was isolated as a white solid (71 mg, 74%).

### (Z)-1-(4-(1-(3,5-Bis(trifluoromethyl)phenyl))-1H-1,2,4-triazol-3-yl)-2-fluorophenyl)-3-(3-(2-(ethoxymethyl)-5-methylphenyl)-4-oxothiazolidin-2-ylidene)urea (J761)

The title compound was prepared and was isolated as a white solid (63 mg, 71%).

### (Z)-1-(4-(1-(3,5-Bis(trifluoromethyl)phenyl)-1H-1,2,4-triazol-3-yl)-2-methylphenyl)-3-(3-(5-methyl-2-((2,2,2-trifluoroethoxy)methyl)phenyl)-4-oxothiazolidin-2-ylidene)urea (J762)

The title compound was prepared and was isolated as a white solid (54 mg, 56%).

### (Z)-1-(4-(1-(3,5-Bis(trifluoromethyl)phenyl)-1H-1,2,4-triazol-3-yl)-2-methylphenyl)-3-(3-(2-(ethoxymethyl)-5-methylphenyl)-4-oxothiazolidin-2-ylidene)urea (J763)

The title compound was prepared and was isolated as a white solid (37 mg, 41%).

### (Z)-1-(2-Fluoro-4-(1-(4-(2,2,2-trifluoroethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J764)

The title compound was prepared and was isolated as a yellow solid (67 mg, 66%).

### (Z)-1-(3-(2-(Ethoxymethyl)-5-methylphenyl)-4-oxothiazolidin-2-ylidene)-3-(2-fluoro-4-(1-(4-(2,2,2-trifluoroethoxy)phenyl)-1H-1,2,4-triazol-3-yl)phenyl)urea (J766)

The title compound was prepared and was isolated as an off-white solid (68 mg, 73%).

### (Z)-1-(2-Fluoro-4-(1-phenyl-1H-1,2,4-triazol-3-yl)phenyl)-3-(3-(5-methyl-2-((2,2,2-trifluoroethoxy)methyl)phenyl)-4-oxothiazolidin-2-ylidene)urea (J769)

The title compound was prepared and was isolated as a white solid (42 mg, 35%).

### (Z)-1-(5,5-Bis(hydroxymethyl)-3-(5-methyl-2-((2,2,2-trifluoroethoxy)methyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J778)

The title compound was prepared and was isolated as a white solid (12 mg, 21%).

### (Z)-1-(2-Fluoro-4-(1-phenyl-1H-1,2,4-triazol-3-yl)phenyl)-3-(3-(5-methyl-2-((3,3,3-trifluoropropoxy)methyl)phenyl)-4-oxothiazolidin-2-ylidene)urea (J779)

The title compound was prepared and was isolated as a white foam (57 mg, 46%).

Z)-1-(3-(5-Methyl-2-((2,2,2-trifluoroethoxy)methyl)phenyl)-4-oxothiazolidin-2-ylidene)-3-(2-methyl-4-(1-phenyl-1H-1,2,4-triazol-3-yl)phenyl)urea (J780)

The title compound was prepared and was isolated as a white foam (63 mg, 50%).

### (Z)-1-(3-(5-Methyl-2-((3,3,3-trifluoropropoxy)methyl)phenyl)-4-oxothiazolidin-2-ylidene)-3-(2-methyl-4-(1-phenyl-1H-1,2,4-triazol-3-yl)phenyl)urea (J781)

The title compound was prepared and was isolated as a white foam (87 mg, 70%).

### (Z)-1-(2-Fluoro-4-(1-(4-fluorophenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J782)

The title compound was prepared and was isolated as a white foam (56 mg, 49%).

### Methyl (Z)-(4-(3-(3-fluoro-4-(3-(3-(5-methyl-2-propylphenyl)-4-oxothiazolidin-2-ylidene)ureido)phenyl)-1H-1,2,4-triazol-1-yl)phenyl)(trifluoromethyl)carbamate (J783)

The title compound was prepared and was isolated as an orange foam (12 mg, 13%).

### Methyl (Z)-(4-(3-(3-methyl-4-(3-(3-(5-methyl-2-propylphenyl)-4-oxothiazolidin-2-ylidene)ureido)phenyl))-1H-1,2,4-triazol-1-yl)phenyl)(trifluoromethyl)carbamate (J784)

The title compound was prepared and was isolated as an orange foam (32 mg, 36%).

### (Z)-1-(2-Fluoro-4-(1-(4-(perfluoroethyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J793)

The title compound was prepared and was isolated as an off-white solid (49 mg, 48%).

### (Z)-1-(3-(5-Methyl-2-((2,2,2-trifluoroethoxy)methyl)phenyl)-4-oxothiazolidin-2-ylidene)-3-(2-methyl-4-(1-(4-(perfluoroethyl)phenyl)-1H-1,2,4-triazol-3-yl)phenyl)urea (J794)

The title compound was prepared and was isolated as a yellow solid (42 mg, 41%).

### (Z)-1-(3-(2-(Ethoxymethyl)-5-methylphenyl)-4-oxothiazolidin-2-ylidene)-3-(2-fluoro-4-(1-(4-(perfluoroethyl)phenyl)-1H-1,2,4-triazol-3-yl)phenyl)urea (J795)

The title compound was prepared and was isolated as an off-white solid (35 mg, 37%).

### (Z)-1-(3-(2-(Ethoxymethyl)-5-methylphenyl)-4-oxothiazolidin-2-ylidene)-3-(2-methyl-4-(1-(4-(perfluoroethyl)phenyl)-1H-1,2,4-triazol-3-yl)phenyl)urea (J796)

The title compound was prepared and was isolated as a yellow solid (53 mg, 56%).

The following compounds were prepared in accordance to the procedure in Example 32.

### (Z)-1-(3-(5-Methyl-2-nitrophenyl)-4-oxothiazolidin-2-ylidene)-3-(4-(1-(4-(trifluoromethoxy)phenyl)-1H-1,2,4-triazol-3-yl)phenyl)urea (J313)

The title compound was prepared and was isolated as a clear colorless oil (660 mg, 71%).

### (Z)-1-(4-(1-(4-(Cyanomethoxy)phenyl)-1H-1,2,4-triazol-3-yl)-2-fluorophenyl)-3-(3-(2-(1-methoxyethyl)-5-methylphenyl)-4-oxothiazolidin-2-ylidene)urea (J340)

The title compound was prepared and was isolated as an off-white powder (63 mg, 46%).

### (Z)-1-(4-(1-(4-(Cyanomethoxy)phenyl)-1H-1,2,4-triazol-3-yl)-2-fluorophenyl)-3-(3-(5-(dimethylamino)-2-isopropylphenyl)-4-oxothiazolidin-2-ylidene)urea (J342)

The title compound was prepared and was isolated as an off-white powder (79 mg, 53%).

### (Z)-1-(4-(1-(4-(Cyanomethoxy)phenyl)-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J343)

The title compound was prepared and was isolated as an off-white powder (82 mg, 52%).

### (Z)-1-(4-(1-(4-(Cyanomethoxy)phenyl)-1H-1,2,4-triazol-3-yl)-2-fluorophenyl)-3-(3-(2-isopropyl-5-methylphenyl)-4-oxothiazolidin-2-ylidene)urea (J344)

The title compound was prepared and was isolated as an off-white powder (72 mg, 51%).

### (Z)-1-(4-(1-(4-(Cyanomethoxy)phenyl)-1H-1,2,4-triazol-3-yl)-2-methylphenyl)-3-(3-(2-isopropyl-5-methylphenyl)-4-oxothiazolidin-2-ylidene)urea (J356)

The title compound was prepared and was isolated as an orange powder (116 mg, 90%).

### (Z)-1-(4-(1-(4-(Cyanomethoxy)phenyl)-1H-1,2,4-triazol-3-yl)-2-methylphenyl)-3-(3-(5-(dimethylamino)-2-isopropylphenyl)-4-oxothiazolidin-2-ylidene)urea (J357)

The title compound was prepared and was isolated as an orange powder (105 mg, 77%).

### (Z)-1-(4-(1-(4-(Cyanomethoxy)phenyl)-1H-1,2,4-triazol-3-yl)-2-methylphenyl)-3-(3-(2-(1-methoxyethyl)-5-methylphenyl)-4-oxothiazolidin-2-ylidene)urea (J358)

The title compound was prepared and was isolated as an orange powder (112 mg, 84%).

### (Z)-1-(4-(1-(4-(Cyanomethoxy)phenyl)-1H-1,2,4-triazol-3-yl)-2-methylphenyl)-3-(3-(5-methyl-2-((2,2,2-trifluoroethoxy)methyl)phenyl)-4-oxothiazolidin-2-ylidene)urea (J359)

The title compound was prepared and was isolated as an orange powder (120 mg, 83%).

### (Z)-1-(4-(1-(4-(Cyanomethoxy)phenyl)-1H-1,2,4-triazol-3-yl)-2-methylphenyl)-3-(3-(5-methyl-2-(3,3,3-trifluoropropoxy)phenyl)-4-oxothiazolidin-2-ylidene)urea (J360)

The title compound was prepared and was isolated as an orange powder (44 mg, 30%).

### (Z)-1-(2-Fluoro-4-(1-(4-(trifluoromethoxy)phenyl)-1H-1,2,4-triazol-3-yl)phenyl)-3-(3-(5-methyl-2-(3-(trifluoromethyl)benzyl)phenyl)-4-oxothiazolidin-2-ylidene)urea (J439)

The title compound was prepared and was isolated as an off-white powder (92 mg, 71%).

### (Z)-1-(3-(5-Methyl-2-(3-(trifluoromethyl)benzyl)phenyl)-4-oxothiazolidin-2-ylidene)-3-(2-methyl-4-(1-(4-(trifluoromethoxy)phenyl)-1H-1,2,4-triazol-3-yl)phenyl)urea (J440)

The title compound was prepared and was isolated as an off-white powder (86 mg, 66%).

### (Z)-1-(3-(2-Benzyl-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J456)

The title compound was prepared and was isolated as an off-white powder (64 mg, 65%).

### (Z)-1-(3-(2-Benzyl-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J457)

The title compound was prepared and was isolated as an off-white powder (80 mg, 82%).

### (Z)-1-(3-(2-(Furan-2-yl)-5-methylphenyl)-4-oxothiazolidin-2-ylidene)-3-(2-methyl-4-(1-(4-(trifluoromethoxy)phenyl)-1H-1,2,4-triazol-3-yl)phenyl)urea (J488)

The title compound was prepared and was isolated as an orange waxy solid (66 mg, 58%).

### (Z)-1-(2-Fluoro-4-(1-(4-(trifluoromethoxy)phenyl)-1H-1,2,4-triazol-3-yl)phenyl)-3-(3-(2-(furan-2-yl)-5-methylphenyl)-4-oxothiazolidin-2-ylidene)urea (J489)

The title compound was prepared and was isolated as an orange waxy solid (63 mg, 56%).

### (Z)-1-(3-(2-Benzyl-5-methylphenyl)-4-oxothiazolidin-2-ylidene)-3-(2-fluoro-4-(1-(4-(trifluoromethyl)phenyl)-1H-1,2,4-triazol-3-yl)phenyl)urea (J562)

The title compound was prepared and was isolated as an off-white solid (59 mg, 39%).

### (Z)-1-(3-(2-Benzyl-5-methylphenyl)-4-oxothiazolidin-2-ylidene)-3-(2-fluoro-4-(1-(4-(pentafluoro-λ⁶-sulfaneyl)phenyl)-1H-1,2,4-triazol-3-yl)phenyl)urea (J563)

The title compound was prepared and was isolated as an off-white solid (46 mg, 36%).

The following compounds were prepared in accordance to the procedure in Example 34.

### (Z)-1-(5-Chloro-3-(2-isopropyl-5-methylphenyl)-4-oxothiazolidin-2-ylidene)-3-(4-(1-(4-(trifluoromethoxy)phenyl)-1H-1,2,4-triazol-3-yl)phenyl)urea (J314)

The title compound was prepared and was isolated as a yellow solid (82 mg, 46%).

### (Z)-1-(5-Chloro-3-(5-methyl-2-(3,3,3-trifluoropropoxy)phenyl)-4-oxothiazolidin-2-ylidene)-3-(4-(1-(4-(trifluoromethyl)phenyl)-1H-1,2,4-triazol-3-yl)phenyl)urea (J317)

The title compound was prepared and was isolated as an off-white foam (15 mg, 8%).

The following compounds were prepared in accordance to the procedure in Example 35.

### (Z)-1-(2-Acetyl-4-(1-(4-(trifluoromethoxy)phenyl)-1H-1,2,4-triazol-3-yl)phenyl)-3-(3-(2-isopropyl-5-methylphenyl)-4-oxothiazolidin-2-ylidene)urea (J395)

The title compound was prepared and was isolated as an off-white powder (67 mg, 64%).

### (Z)-1-(2-Acetyl-4-(1-(4-(trifluoromethoxy)phenyl)-1H-1,2,4-triazol-3-yl)phenyl)-3-(3-(5-(dimethylamino)-2-isopropylphenyl)-4-oxothiazolidin-2-ylidene)urea (J396)

The title compound was prepared and was isolated as an off-white powder (71 mg, 64%).

### (Z)-1-(2-Acetyl-4-(1-(4-(trifluoromethoxy)phenyl)-1H-1,2,4-triazol-3-yl)phenyl)-3-(3-(2-(1-methoxyethyl)-5-methylphenyl)-4-oxothiazolidin-2-ylidene)urea (J397)

The title compound was prepared and was isolated as an off-white powder (65 mg, 60%).

### (Z)-1-(2-Acetyl-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J398)

The title compound was prepared and was isolated as an off-white powder (63 mg, 54%).

### (Z)-1-(2-Acetyl-4-(1-(4-(trifluoromethoxy)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J399)

The title compound was prepared and was isolated as an off-white powder (69 mg, 59%).

### (Z)-1-(2-Acetyl-4-(1-(5-(trifluoromethyl)pyridin-2-yl)-1H-1,2,4-triazol-3-yl)phenyl)-3-(3-(2-isopropyl-5-methylphenyl)-4-oxothiazolidin-2-ylidene)urea (J415)

The title compound was prepared and was isolated as an off-white powder (43 mg, 40%).

### (Z)-1-(2-Acetyl-4-(1-(5-(trifluoromethyl)pyridin-2-yl)-1H-1,2,4-triazol-3-yl)phenyl)-3-(3-(2-(1-methoxyethyl)-5-methylphenyl)-4-oxothiazolidin-2-ylidene)urea (J416)

The title compound was prepared and was isolated as an off-white powder (35 mg, 32%).

### (Z)-1-(2-Acetyl-4-(1-(5-(trifluoromethyl)pyridin-2-yl)-1H-1,2,4-triazol-3-yl)phenyl)-3-(3-(5-(dimethylamino)-2-isopropylphenyl)-4-oxothiazolidin-2-ylidene)urea (J417)

The title compound was prepared and was isolated as an off-white powder (25 mg, 22%).

### (Z)-1-(2-Acetyl-4-(1-(5-(trifluoromethyl)pyridin-2-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J418)

The title compound was prepared and was isolated as an off-white powder (33 mg, 28%).

### (Z)-1-(2-Acetyl-4-(1-(5-(trifluoromethyl)pyridin-2-yl)-1H-1,2,4-triazol-3-yl)phenyl)-3-(3-(5-(dimethylamino)-2-propylphenyl)-4-oxothiazolidin-2-ylidene)urea (J419)

The title compound was prepared and was isolated as an off-white powder (21 mg, 19%).

### (Z)-1-(4-(1-(4-(Cyanomethyl)phenyl)-1H-1,2,4-triazol-3-yl)-2-fluorophenyl)-3-(3-(5-methyl-2-((2,2,2-trifluoroethoxy)methyl)phenyl)-4-oxothiazolidin-2-ylidene)urea (J425)

The title compound was prepared and was isolated as a brown foam (90 g, 71%).

### (Z)-1-(2-Cyano-4-(1-(4-(pentafluoro-λ⁶-sulfaneyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J569)

The title compound was prepared and was isolated as a light yellow solid (54 mg, 48%).

### (Z)-1-(2-Cyano-4-(1-(4-(pentafluoro-)⁶-sulfaneyl)phenyl)-1H-1,2,4-triazol-3-yl)phenyl)-3-(3-(2-(ethoxymethyl)-5-methylphenyl)-4-oxothiazolidin-2-ylidene)urea (J582)

The title compound was prepared and was isolated as a light brown solid (68 mg, 65%).

### (Z)-1-(2-Cyano-4-(1-(4-(pentafluoro-λ⁶-sulfaneyl)phenyl)-1H-1,2,4-triazol-3-yl)phenyl)-3-(3-(5-methyl-2-propylphenyl)-4-oxothiazolidin-2-ylidene)urea (J583)

The title compound was prepared and was isolated as a white solid (44 mg, 42%).

### (Z)-1-(2-Cyano-4-(1-(4-(pentafluoro-λ⁶-sulfaneyl)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J584)

The title compound was prepared and was isolated as a light brown solid (92 mg, 81%).

### (Z)-1-(2-Cyano-4-(1-(4-(pentafluoro-)-λ⁶-sulfaneyl)phenyl)-1H-1,2,4-triazol-3-yl)phenyl)-3-(3-(5-(dimethylamino)-2-isopropylphenyl)-4-oxothiazolidin-2-ylidene)urea (J585)

The title compound was prepared and was isolated as a light brown solid (80 mg, 75%).

### (Z)-1-(2-Cyano-4-(1-(4-(pentafluoro-λ⁶-sulfaneyl)phenyl)-1H-1,2,4-triazol-3-yl)phenyl)-3-(3-(2-(1-ethoxyethyl)-5-methylphenyl)-4-oxothiazolidin-2-ylidene)urea (J586)

The title compound was prepared and was isolated as a pink foam (62 mg, 58%).

### (Z)-1-(2-Cyano-4-(1-(4-(pentafluoro-λ⁶-sulfaneyl)phenyl)-1H-1,2,4-triazol-3-yl)phenyl)-3-(3-(4,5-dimethyl-2-((2,2,2-trifluoroethoxy)methyl)phenyl)-4-oxothiazolidin-2-ylidene)urea (J587)

The title compound was prepared and was isolated as a light brown solid (88 mg, 76%).

### (Z)-1-(2-Cyano-4-(1-(4-(pentafluoro-λ⁶-sulfaneyl)phenyl)-1H-1,2,4-triazol-3-yl)phenyl)-3-(3-(4,5-dimethoxy-2-((2,2,2-trifluoroethoxy)methyl)phenyl)-4-oxothiazolidin-2-ylidene)urea (J588)

The title compound was prepared and was isolated as a light brown solid (79 mg, 65%).

### (Z)-1-(3-(5-Methyl-2-((2,2,2-trifluoroethoxy)methyl)phenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J626)

The title compound was prepared and was isolated as a yellow foam (57 mg, 53%).

### (Z)-1-(3-(2-(Ethoxymethyl)-5-methylphenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J627)

The title compound was prepared and was isolated as a yellow foam (49 mg, 49%).

### (Z)-1-(3-(5-Methyl-2-propylphenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J628)

The title compound was prepared and was isolated as a yellow foam (49 mg, 50%).

### (Z)-1-(3-(5-Methyl-2-(3,3,3-trifluoropropoxy)phenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J629)

The title compound was prepared and was isolated as an off-white foam (60 mg, 56%).

### (Z)-1-(3-(5-(Dimethylamino)-2-isopropylphenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J630)

The title compound was prepared and was isolated as a yellow foam (63 mg, 62%).

### (Z)-1-(3-(2-(1-Ethoxyethyl)-5-methylphenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J631)

The title compound was prepared and was isolated as a yellow foam (65 mg, 63%).

### (Z)-1-(3-(4,5-Dimethyl-2-((2,2,2-trifluoroethoxy)methyl)phenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J632)

The title compound was prepared and was isolated as a white foam (64 mg, 58%).

### (Z)-1-(3-(4,5-Dimethoxy-2-((2,2,2-trifluoroethoxy)methyl)phenyl)-4-oxothiazolidin-2-ylidene)-3-(4-(1-(4-(pentafluoro-λ⁶-sulfaneyl)phenyl)-1H-1,2,4-triazol-3-yl)-2-(trifluoromethyl)phenyl)urea (J633)

The title compound was prepared and was isolated as a yellow foam (65 mg, 57%).

### (Z)-1-(2-Fluoro-4-(1-(4-(trifluoromethyl)phenyl)-1H-1,2,4-triazol-3-yl)phenyl)-3-(3-(2-(4-fluorobenzyl)-5-methylphenyl)-4-oxothiazolidin-2-ylidene)urea (J673)

The title compound was prepared and was isolated as a white solid (109 mg, 88%).

### (Z)-1-(2-Fluoro-4-(1-(4-(trifluoromethoxy)phenyl)-1H-1,2,4-triazol-3-yl)phenyl)-3-(3-(2-(4-fluorobenzyl)-5-methylphenyl)-4-oxothiazolidin-2-ylidene)urea (J674)

The title compound was prepared and was isolated as a white solid (89 mg, 74%).

### (Z)-1-(2-Fluoro-4-(1-(4-(trifluoromethyl)phenyl)-1H-1,2,4-triazol-3-yl)phenyl)-3-(3-(2-(3-fluorobenzyl)-5-methylphenyl)-4-oxothiazolidin-2-ylidene)urea (J675)

The title compound was prepared and was isolated as a white solid (114 mg, 92%).

### (Z)-1-(2-Fluoro-4-(1-(4-(trifluoromethoxy)phenyl)-1H-1,2,4-triazol-3-yl)phenyl)-3-(3-(2-(3-fluorobenzyl)-5-methylphenyl)-4-oxothiazolidin-2-ylidene)urea (J676)

The title compound was prepared and was isolated as a white solid (110 mg, 91%).

### (Z)-1-(2-Cyano-4-(1-(4-(pentafluoro-λ⁶-sulfaneyl)phenyl)-1H-1,2,4-triazol-3-yl)phenyl)-3-(3-(2-(4-fluorobenzyl)-5-methylphenyl)-4-oxothiazolidin-2-ylidene)urea (J683)

The title compound was prepared and was isolated as a light yellow solid (55 mg, 39%).

### (Z)-1-(2-Fluoro-4-(1-(4-(trifluoromethyl)phenyl)-1H-1,2,4-triazol-3-yl)phenyl)-3-(3-(2-(2-fluorobenzyl)-5-methylphenyl)-4-oxothiazolidin-2-ylidene)urea (J708)

The title compound was prepared and was isolated as a tan solid (50 mg, 53%).

### (Z)-1-(2-Fluoro-4-(1-(2-(2,2,2-trifluoroethoxy)pyridin-4-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J750)

The title compound was prepared and was isolated as a white solid (37 mg, 86%).

### (Z)-1-(2-Fluoro-4-(1-(2-oxo-1-(2,2,2-trifluoroethyl)-1,2-dihydropyridin-4-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J751)

The title compound was prepared and was isolated as a tan solid (59 mg, 86%).

### (Z)-1-(2-Fluoro-4-(1-(4-(trifluoromethyl)phenyl)-1H-1,2,4-triazol-3-yl)phenyl)-3-(3-(5-methyl-2-(pyridin-3-ylmethyl)phenyl)-4-oxothiazolidin-2-ylidene)urea (J758)

The title compound was prepared and was isolated as a white solid (58 mg, 67%).

### (Z)-1-(2-Fluoro-4-(1-(4-(trifluoromethoxy)phenyl)-1H-1,2,4-triazol-3-yl)phenyl)-3-(3-(5-methyl-2-(pyridin-3-ylmethyl)phenyl)-4-oxothiazolidin-2-ylidene)urea (J759)

The title compound was prepared and was isolated as a white solid (63 mg, 71%).

### (Z)-1-(2-Fluoro-4-(1-(6-(2,2,2-trifluoroethoxy)pyridin-3-yl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J765)

The title compound was prepared and was isolated as a light brown solid (63 mg, 71%).

The following compounds were prepared in accordance to the procedure in Example 36:

### 3-(2-(4-Fluorobenzyl)-5-methylphenyl)-2-iminothiazolidin-4-one (C348)

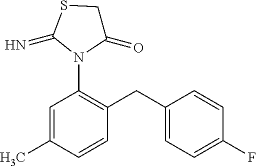 [SMILES: Cc1ccc(Cc2ccc(F)cc2)c(N2C(=N)SCC2=O)c1]

The title compound was prepared and was isolated as a colorless glass (187 mg, 85%): ¹H NMR (400 MHz, CDCl₃) δ 7.81 (s, 1H), 7.26 (t, J=1.6 Hz, 1H), 7.24-7.12 (m, 2H), 7.12-7.00 (m, 2H), 6.99-6.87 (m, 2H), 3.96-3.71 (m, 4H), 2.36 (s, 3H); ¹⁹F NMR (376 MHz, CDCl₃) δ −116.75; ESIMS m/z 315 ([M+H]⁺).

### 3-(2-(3-Fluorobenzyl)-5-methylphenyl)-2-iminothiazolidin-4-one (C349)

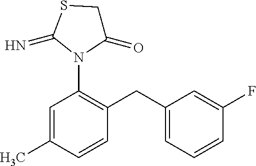 [SMILES: Cc1ccc(Cc2cccc(F)c2)c(N2C(=N)SCC2=O)c1]

The title compound was prepared and was isolated as a colorless oil (169 mg, 87%): ¹H NMR (400 MHz, CDCl₃) δ 7.82 (s, 1H), 7.26 (t, J=1.6 Hz, 1H), 7.24-7.15 (m, 2H), 7.00-6.80 (m, 4H), 3.99-3.70 (m, 4H), 2.36 (s, 3H); ¹⁹F NMR (376 MHz, CDCl₃) δ −113.33; ESIMS m/z 315 ([M+H]⁺).

### 3-(2-(2-Fluorobenzyl)-5-methylphenyl)-2-iminothiazolidin-4-one (C350)

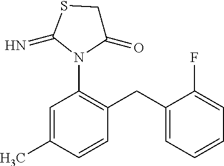 [SMILES: Cc1ccc(Cc2ccccc2F)c(N2C(=N)SCC2=O)c1]

The title compound was prepared and was isolated as a yellow oil (46 mg, 34%): ¹H NMR (400 MHz, CDCl₃) δ 7.28-7.12 (m, 3H), 7.12-6.92 (m, 4H), 4.00-3.87 (m, 2H), 3.85 (s, 2H), 2.35 (s, 3H); ¹⁹F NMR (376 MHz, CDCl₃) δ −117.74; ESIMS m/z 315 ([M+H]⁺).

### 2-Imino-3-(5-methyl-2-(pyridin-3-ylmethyl)phenyl)thiazolidin-4-one (C351)

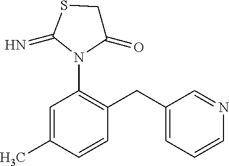 [SMILES: Cc1ccc(Cc2cccnc2)c(N2C(=N)SCC2=O)c1]

The title compound was prepared and was isolated as an amber oil (46 mg, 34%): ¹H NMR (400 MHz, CDCl₃) δ 8.46 (d, J=7.9 Hz, 2H), 7.39 (dt, J=7.9, 2.0 Hz, 1H), 7.30-7.13 (m, 4H), 7.00-6.93 (m, 1H), 3.92 (d, J=17.0 Hz, 1H), 3.87-3.75 (m, 3H), 2.36 (s, 3H); ESIMS m/z 298 ([M+H]⁺).

### 3-(4,5-Dimethyl-2-((2,2,2-trifluoroethoxy)methyl)phenyl)-2-iminothiazolidin-4-one (C352)

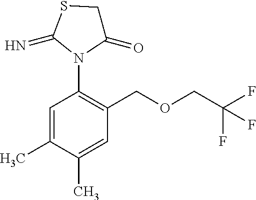 [SMILES: Cc1cc(COCC(F)(F)F)c(N2C(=N)SCC2=O)cc1C]

The title compound was prepared and was isolated as a light orange oil (979 mg, 81%): ¹H NMR (400 MHz, CDCl₃) δ 7.80 (s, 1H), 6.98 (s, 1H), 4.59 (d, J=12.9 Hz, 1H), 4.46 (d, J=12.4 Hz, 1H), 4.06 (d, J=3.6 Hz, 2H), 3.70 (qd, J=8.8, 1.7 Hz, 2H), 2.30 (s, 3H), 2.29 (s, 3H); ¹⁹F NMR (376 MHz, CDCl₃) δ −73.79.

### 3-(4,5-Dimethoxy-2-((2,2,2-trifluoroethoxy)methyl)phenyl)-2-iminothiazolidin-4-one (C353)

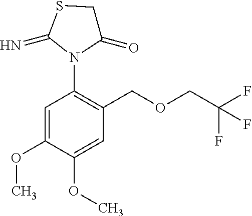 [SMILES: COc1cc(COCC(F)(F)F)c(N2C(=N)SCC2=O)cc1OC]

The title compound was prepared and was isolated as a light red solid (734 mg, 78%): ¹H NMR (400 MHz, CDCl₃) δ 7.85 (s, 1H), 7.00 (s, 1H), 6.67 (s, 1H), 4.56 (d, J=12.9 Hz, 1H), 4.46 (d, J=12.2 Hz, 1H), 4.07 (s, 2H), 3.92 (s, 3H), 3.88 (s, 3H), 3.73 (q, J=8.8 Hz, 2H); ¹⁹F NMR (376 MHz, CDCl₃) δ −73.77.

### 2-Imino-3-(5-methyl-2-propoxyphenyl)thiazolidin-4-one (C354)

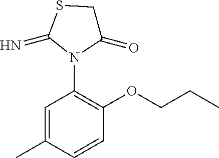 [SMILES: CCCOc1ccc(C)cc1N1C(=N)SCC1=O]

The title compound was prepared and was isolated as a yellow solid (1.7 g, 40%): mp 78-82° C.; ¹H NMR (400 MHz, DMSO-d₆) δ 9.11 (s, 1H), 7.17 (dd, J=1.6, 8.4 Hz, 1H), 7.02 (d, J=8.8 Hz, 1H), 6.94 (d, J=2.0 Hz, 1H), 4.18-4.07 (m, 2H), 3.87 (t, J=8.0 Hz, 2H), 2.25 (s, 3H), 1.65-1.56 (m, 2H), 0.88 (t, J=7.6 Hz, 3H); ESIMS m/z 265 ([M+H]⁺).

### 2-Imino-3-(2-(1-methoxypropyl)-5-methylphenyl)thiazolidin-4-one (C355)

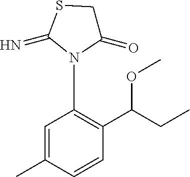 [SMILES: CCC(OC)c1ccc(C)cc1N1C(=N)SCC1=O]

The title compound was prepared and was isolated as a pale yellow solid (0.6 g, 39% over two steps from NCG-46a): mp 75-79° C.; ¹H NMR (400 MHz, DMSO-d₆) δ 9.29-9.23 (m, 1H), 7.33-7.26 (m, 2H), 6.94 (s, 1H), 4.31-4.12 (m, 2H), 3.92-3.85 (m, 1H), 3.01-2.99 (m, 3H), 2.33 (s, 3H), 1.57-1.45 (m, 2H), 0.86-0.80 (m, 3H); ESIMS m/z 279 ([M+H]⁺).

### 3-(2,3-Dimethyl-6-((2,2,2-trifluoroethoxy)methyl)phenyl)-2-iminothiazolidin-4-one (C356)

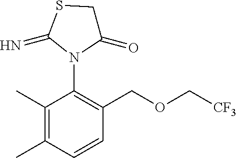 [SMILES: Cc1ccc(COCC(F)(F)F)c(N2C(=N)SCC2=O)c1C]

The title compound was prepared and was isolated as a pale brown solid (1.2 g, 52%): mp 81-85° C.; ¹H NMR (400 MHz, DMSO-d₆) δ 9.24 (s, 1H), 7.28 (d, J=7.6 Hz, 1H), 7.23 (d, J=7.6 Hz, 1H), 4.42 (q, J=12.4 Hz, 2H), 4.32-4.14 (m, 2H), 3.92 (q, J=9.2 Hz, 2H), 2.28 (s, 3H), 1.93 (s, 3H); ¹⁹F NMR (376 MHz, DMSO-d₆) δ −72.70; ESIMS m/z 333 ([M+H]⁺).

### 2-Imino-3-(5-methyl-2-((2,2,2-trichloroethoxy)methyl)phenyl)thiazolidin-4-one (C357)

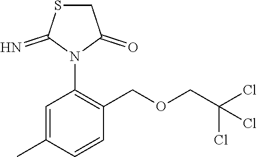 [SMILES: Cc1ccc(COCC(Cl)(Cl)Cl)c(N2C(=N)SCC2=O)c1]

The title compound was prepared and was isolated as an orange oil (799 mg, 86%): ¹H NMR (400 MHz, CDCl₃) δ 7.84 (s, 1H), 7.46 (d, J=7.8 Hz, 1H), 7.30 (d, J=8.1, 1H), 7.04 (s, 1H), 4.89-4.56 (m, 2H), 4.16-4.03 (m, 2H), 3.98 (s, 2H), 2.40 (s, 3H); ESIMS m/z 368 ([M+H]⁺).

### 3-(2-((2,2-Difluoropropoxy)methyl)-5-methylphenyl)-2-iminothiazolidin-4-one (C358)

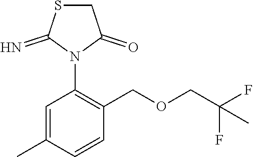 [SMILES: Cc1ccc(COCC(C)(F)F)c(N2C(=N)SCC2=O)c1]

The title compound was prepared and was isolated as an orange oil (1.24 g, 79%): ¹H NMR (400 MHz, CDCl₃) δ 7.81 (s, 1H), 7.40 (s, 1H), 7.29 (s, 1H), 7.02 (s, 1H), 4.64-4.36 (m, 2H), 4.10-3.94 (m, 2H), 3.51 (t, J=12.4 Hz, 2H), 2.39 (s, 3H), 1.60 (s, 3H); ESIMS m/z 315 ([M+H]⁺).

### 2-Imino-3-(5-methyl-2-((3,3,3-trifluoropropoxy)methyl)phenyl)thiazolidin-4-one (RS-36c)

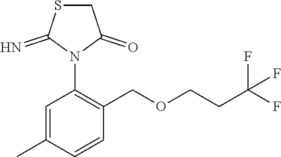 [SMILES: Cc1ccc(COCCC(F)(F)F)c(N2C(=N)SCC2=O)c1]

The title compound was prepared and was isolated as an orange oil (1 g, 87%): ¹H NMR (400 MHz, CDCl₃) δ 7.81 (s, 1H), 7.38 (d, J=7.6 Hz, 2H), 7.02 (s, 1H), 4.42 (dd, J=55.9, 12.2 Hz, 2H), 4.07 (s, 2H), 3.54 (t, J=6.6 Hz, 2H), 2.39 (m, 5H); ¹⁹F NMR (376 MHz, CDCl₃) δ −64.67; ESIMS m/z 333 ([M+H]⁺).

### 2-Imino-3-(5-methyl-2-(propoxymethyl)phenyl)thiazolidin-4-one (C359)

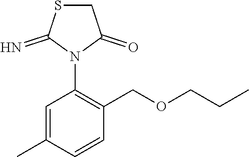 [SMILES: CCCOCc1ccc(C)cc1N1C(=N)SCC1=O]

The title compound was prepared and was isolated as an orange oil (799 mg, 87%): ¹H NMR (400 MHz, CDCl₃) δ 7.80 (s, 1H), 7.40 (s, 1H), 7.00 (s, 1H), 4.53-4.21 (m, 2H), 4.06 (s, 2H), 3.30 (t, J=6.8 Hz, 2H), 2.38 (s, 3H), 1.57 (p, J=7.2 Hz, 2H), 0.89 (t, J=7.4 Hz, 3H); ESIMS m/z 279 ([M+H]⁺).

### 3-(2-((2,2-Dichloroethoxy)methyl)-5-methylphenyl)-2-iminothiazolidin-4-one (C360)

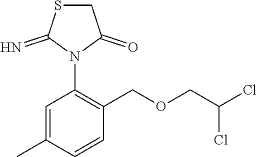 [SMILES: Cc1ccc(COCC(Cl)Cl)c(N2C(=N)SCC2=O)c1]

The title compound was prepared and was isolated as an orange oil (1.03 g, 88%): ¹H NMR (400 MHz, CDCl₃) δ 7.82 (s, 1H), 7.42 (d, J=7.8 Hz, 1H), 7.29 (d, J=7.8 Hz, 1H), 7.03 (s, 1H), 5.68 (t, J=6.0 Hz, 1H), 4.58 (s, 1H), 4.46 (d, J=12.5 Hz, 1H), 4.17-3.97 (m, 2H), 3.80-3.64 (m, 2H), 2.40 (s, 3H); ESIMS m/z 334 ([M+H]⁺).

### 3-(2-((2-Fluoroethoxy)methyl)-5-methylphenyl)-2-iminothiazolidin-4-one (C361)

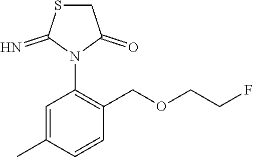 [SMILES: Cc1ccc(COCCF)c(N2C(=N)SCC2=O)c1]

The title compound was prepared and was isolated as an orange oil (553 mg, 81%): ¹H NMR (400 MHz, CDCl₃) δ 7.77 (s, 1H), 7.40 (d, J=7.8 Hz, 1H), 7.28 (d, J=6.8 Hz, 1H), 7.06-6.96 (m, 1H), 4.62-4.48 (m, 2H), 4.48-4.35 (m, 2H), 4.07 (q, J=16.8 Hz, 2H), 3.68-3.44 (m, 2H), 2.39 (s, 3H); ¹⁹F NMR (376 MHz, CDCl₃) δ 18.52; ESIMS m/z 283 ([M+H]⁺).

### 2-Imino-3-(2-((2,2,2-trifluoroethoxy)methyl)phenyl)thiazolidin-4-one (C362)

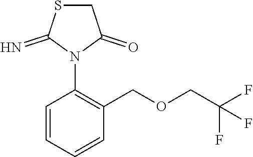 [SMILES: N=C1SCC(=O)N1c1ccccc1COCC(F)(F)F]

The title compound was prepared and was isolated as an orange oil (4.08 g, 74%): ¹H NMR (400 MHz, CDCl₃) δ 7.99-7.70 (m, 1H), 7.71-7.45 (m, 3H), 7.23 (d, J=6.5 Hz, 1H), 4.77-4.45 (m, 2H), 4.08 (s, 2H), 3.73 (q, J=8.9 Hz, 2H); ¹⁹F NMR (376 MHz, CDCl₃) δ −73.81; ESIMS m/z 305 ([M+H]⁺).

### 3-(5-Ethyl-2-((2,2,2-trifluoroethoxy)methyl)phenyl)-2-iminothiazolidin-4-one (C363)

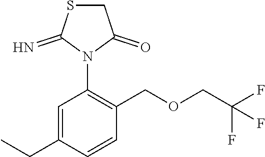 [SMILES: CCc1ccc(COCC(F)(F)F)c(N2C(=N)SCC2=O)c1]

The title compound was prepared and was isolated as a yellow oil (613 mg, 78%): ¹H NMR (400 MHz, CDCl₃) δ 7.82 (s, 1H), 7.37 (d, J=30.6 Hz, 2H), 7.05 (s, 1H), 4.76-4.41 (m, 2H), 4.12-3.95 (m, 2H), 3.71 (q, J=8.7 Hz, 2H), 2.71 (q, J=7.6 Hz, 2H), 1.32-1.22 (m, 3H); ¹⁹F NMR (376 MHz, CDCl₃) δ −73.79; ESIMS m/z 333 ([M+H]⁺).

### 2-Imino-3-(4-methyl-2-((2,2,2-trifluoroethoxy)methyl)phenyl)thiazolidin-4-one (C364)

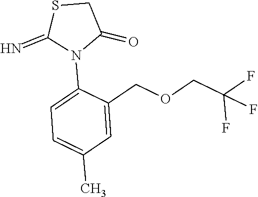 [SMILES: Cc1ccc(N2C(=N)SCC2=O)c(COCC(F)(F)F)c1]

The title compound was prepared and was isolated as an orange oil (1.9 g, 78%): ¹H NMR (400 MHz, CDCl₃) δ 7.82 (s, 1H), 7.40-7.27 (m, 2H), 7.10 (d, J=7.9 Hz, 1H), 4.71-4.42 (m, 2H), 4.06 (d, J=2.4 Hz, 2H), 3.72 (q, J=8.8 Hz, 2H), 2.41 (s, 3H); ¹⁹F NMR (376 MHz, CDCl₃) δ −73.80.

### 3-(2-((2,2-Difluoroethoxy)methyl)-5-methylphenyl)-2-iminothiazolidin-4-one (C365)

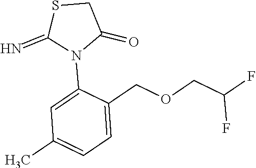 [SMILES: Cc1ccc(COCC(F)F)c(N2C(=N)SCC2=O)c1]

The title compound was prepared and was isolated as a red oil (6 g, 70%): ¹H NMR (400 MHz, CDCl₃) δ 7.82 (s, 1H), 7.42-7.28 (m, 2H), 7.03 (s, 1H), 5.81 (t, J=55.4 Hz, 1H), 4.66-4.34 (m, 2H), 4.09-3.97 (m, 2H), 3.55 (td, J=14.1, 4.0 Hz, 2H), 2.40 (s, 3H); ¹⁹F NMR (376 MHz, CDCl₃) δ −125.03.

### 3-(2-((2-Chloro-2,2-difluoroethoxy)methyl)-5-methylphenyl)-2-iminothiazolidin-4-one (C366)

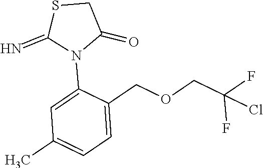 [SMILES: Cc1ccc(COCC(F)(F)Cl)c(N2C(=N)SCC2=O)c1]

The title compound was prepared and was isolated as a yellow oil (588 mg, 70%): ¹H NMR (400 MHz, CDCl₃) δ 7.82 (s, 1H), 7.41 (d, J=7.8 Hz, 1H), 7.30 (d, J=7.7 Hz, 1H), 7.03 (s, 1H), 4.65 (d, J=12.7 Hz, 1H), 4.53 (d, J=12.4 Hz, 1H), 4.07 (d, J=4.6 Hz, 2H), 3.80 (t, J=11.4 Hz, 2H), 2.40 (s, 3H); ¹⁹F NMR (376 MHz, CDCl₃) δ, −61.22.

### 2-Imino-3-(3-methyl-2-((2,2,2-trifluoroethoxy)methyl)phenyl)thiazolidin-4-one (C367)

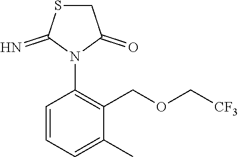 [SMILES: Cc1cccc(N2C(=N)SCC2=O)c1COCC(F)(F)F]

The title compound was prepared and was isolated as a pale brown solid (2.0 g, 57%): mp 97-100° C.; ¹H NMR (400 MHz, DMSO-d₆) δ 9.25 (s, 1H), 7.39-7.24 (m, 2H), 7.03 (dd, J=0.8, 7.6 Hz, 1H), 4.48 (q, J=12.0 Hz, 2H), 4.17-4.16 (m, 2H), 3.93-3.84 (m, 2H), 2.40 (s, 3H); ¹⁹F NMR (376 MHz, DMSO-d₆) δ−72.67; ESIMS m/z 319 ([M+H]⁺).

### 2-Imino-3-(5-methoxy-2-((2,2,2-trifluoroethoxy)methyl)phenyl)thiazolidin-4-one (C368)

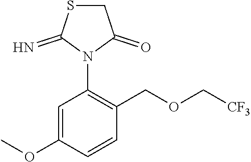 [SMILES: COc1ccc(COCC(F)(F)F)c(N2C(=N)SCC2=O)c1]

The title compound was prepared and was isolated as a pale brown solid (2.1 g, 33%): mp 70-74° C.; ¹H NMR (400 MHz, DMSO-d₆) δ 9.25 (s, 1H), 7.42 (d, J=8.4 Hz, 1H), 7.05 (dd, J=2.8, 8.4 Hz, 1H), 6.83 (d, J=2.8 Hz, 1H), 4.48-4.37 (m, 2H), 4.13 (s, 2H), 3.91 (q, J=9.2 Hz, 2H), 3.77 (s, 3H); ¹⁹F NMR (376 MHz, DMSO-d₆) δ −72.71; ESIMS m/z 335 ([M+H]⁺).

### 3-(5-Fluoro-2-((2,2,2-trifluoroethoxy)methyl)phenyl)-2-iminothiazolidin-4-one (C369)

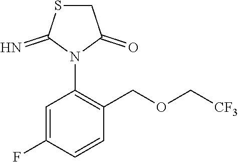 [SMILES: N=C1SCC(=O)N1c1cc(F)ccc1COCC(F)(F)F]

The title compound was prepared and was isolated as an off-white solid (2.2 g, 45%): mp 136-139° C.; ¹H NMR (400 MHz, DMSO-d₆) δ 9.32 (s, 1H), 7.58-7.51 (m, 1H), 7.39-7.31 (m, 1H), 7.23 (dd, J=2.8, 9.2 Hz, 1H), 4.58-4.37 (m, 2H), 4.21-4.05 (m, 2H), 3.98 (q, J=9.2 Hz, 2H); ¹⁹F NMR (376 MHz, DMSO-d₆) δ−72.78, −113.38; ESIMS m/z 323 ([M+H]⁺).

### 3-(2-Imino-4-oxothiazolidin-3-yl)-4-(1-methoxyethyl) benzonitrile (C370)

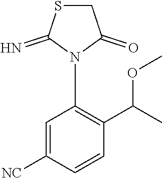 [SMILES: COC(C)c1ccc(C#N)cc1N1C(=N)SCC1=O]

The title compound was prepared and was isolated as a pale brown solid (0.61 g, 55% over 2 steps from NCG-64c): mp 176-180° C.; ¹H NMR (400 MHz, DMSO-d₆) δ 9.41-9.35 (m, 1H), 7.96 (dd, J=1.2, 8.0 Hz, 1H), 7.82-7.80 (m, 1H), 7.96 (d, J=7.6 Hz, 1H), 4.35-4.25 (m, 2H), 4.15-4.11 (m, 1H), 3.02-3.00 (d, 3H), 1.26-1.20 (m, 3H); ESIMS m/z 276 ([M+H]⁺).

### 3-(5-(Dimethylamino)-2-(methoxymethyl)phenyl)-2-iminothiazolidin-4-one (C371)

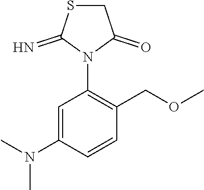

The title compound was prepared and was isolated as a pale brown solid (1.5 g, 48% over 2 steps from NCG-64b): mp 113-117° C.; ¹H NMR (400 MHz, DMSO-d₆) δ 9.15 (s, 1H), 7.24 (d, J=8.8 Hz, 1H), 6.76 (dd, J=2.4, 8.8 Hz, 1H), 6.47 (dd, J=3.2, 8.0 Hz, 1H), 4.18-4.06 (m, 4H), 3.11 (s, 3H), 2.88 (s, 6H); ESIMS m/z 280 ([M+H]⁺).

The following compounds were prepared in accordance to the procedure in Example 37:

### 3-(2-Benzyl-5-methylphenyl)-2-iminothiazolidin-4-one (C372)

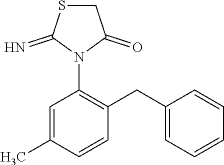 [SMILES: Cc1ccc(Cc2ccccc2)c(N2C(=N)SCC2=O)c1]

The title compound was prepared and was isolated as a tan solid (0.343 g, 71%): ESIMS m/z 297 ([M+H]⁺).

### 2-Imino-3-(5-methyl-2-(3-(trifluoromethyl)benzyl)phenyl)thiazolidin-4-one (C373)

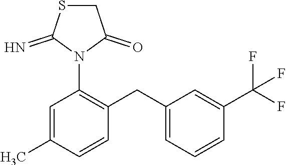 [SMILES: Cc1ccc(Cc2cccc(C(F)(F)F)c2)c(N2C(=N)SCC2=O)c1]

The title compound was prepared and was isolated as a tan solid (0.325 g, 92%): ¹H NMR (400 MHz, CDCl₃) δ 7.81 (s, 1H), 7.48-7.43 (m, 2H), 7.43-7.32 (m, 1H), 7.19 (s, 2H), 6.98-6.90 (m, 1H), 3.98-3.83 (m, 4H), 3.76-3.64 (m, 1H), 2.37 (s, 3H); ¹⁹F NMR (376 MHz, CDCl₃) δ −62.55.

### 3-(2-(Furan-2-yl)-5-methylphenyl)-2-iminothiazolidin-4-one (C374)

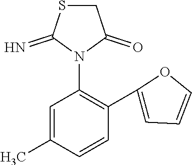 [SMILES: Cc1ccc(-c2ccco2)c(N2C(=N)SCC2=O)c1]

The title compound was prepared and was isolated as a tan solid (0.189 g, 46%): ¹H NMR (400 MHz, CDCl₃) δ 7.77 (d, J=9.7 Hz, 1H), 7.71 (d, J=8.1 Hz, 1H), 7.43 (d, J=6.9 Hz, 1H), 7.30 (d, J=8.1 Hz, 1H), 7.04 (d, J=15.3 Hz, 1H), 6.47 (dd, J=3.4, 0.8 Hz, 1H), 6.46-6.41 (m, 1H), 4.05 (d, J=19.7 Hz, 2H), 2.40 (s, 3H).

The following compounds were prepared in accordance to the procedure in Example 41:

### N-(2-Benzyl-5-methylphenyl)-2-chloroacetamide (C375)

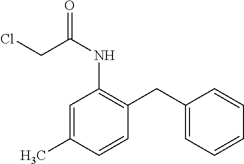 [SMILES: Cc1ccc(Cc2ccccc2)c(NC(=O)CCl)c1]

The title compound was prepared and was isolated as a yellow solid (0.447 g, 63%): ¹H NMR (400 MHz, CDCl₃) δ 7.95 (s, 1H), 7.68 (s, 1H), 7.35-7.20 (m, 3H), 7.15 (dd, J=7.7, 4.1 Hz, 3H), 7.01 (d, J=8.0 Hz, 1H), 4.06 (s, 2H), 3.98 (s, 2H), 2.36 (s, 3H).

### 2-Chloro-N-(5-methyl-2-(3-(trifluoromethyl)benzyl)phenyl)acetamide (C376)

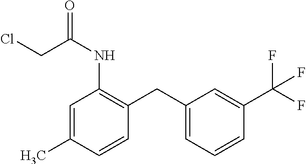 [SMILES: Cc1ccc(Cc2cccc(C(F)(F)F)c2)c(NC(=O)CCl)c1]

The title compound was prepared and was isolated as a yellow solid (0.343 g, 61%): ¹H NMR (400 MHz, CDCl₃) δ 7.90 (s, 1H), 7.62 (s, 1H), 7.49 (d, J=7.9 Hz, 1H), 7.45 (s, 1H), 7.40 (t, J=7.7 Hz, 1H), 7.30 (d, J=7.7 Hz, 1H), 7.14 (d, J=7.7 Hz), 7.04 (d, J=7.7 Hz, 1H), 4.09 (s, 2H), 4.02 (s, 2H), 2.37 (s, 3H); ¹⁹F NMR (376 MHz, CDCl₃) δ −62.62.

### 2-Chloro-N-(2-(furan-2-yl)-5-methylphenyl)acetamide (C377)

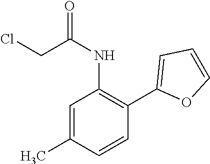 [SMILES: Cc1ccc(-c2ccco2)c(NC(=O)CCl)c1]

The title compound was prepared and was isolated as a yellow solid (0.362 g, 65%): ¹H NMR (400 MHz, CDCl₃) δ 9.65 (s, 1H), 8.17 (d, J=1.6 Hz, 1H), 7.55 (dd, J=1.9, 0.8 Hz, 1H), 7.45 (d, J=8.0 Hz, 1H), 7.01 (d, J=8.0 Hz, 1H), 6.61 (dd, J=3.4, 0.8 Hz, 1H), 6.54 (dd, J=3.4, 1.8 Hz, 1H), 4.22 (s, 2H), 2.39 (s, 3H).

### 2-Chloro-N-(2-(4-fluorobenzyl)-5-methylphenyl)acetamide (C378)

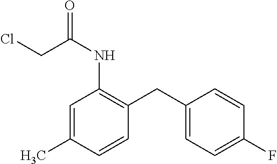 [SMILES: Cc1ccc(Cc2ccc(F)cc2)c(NC(=O)CCl)c1]

The title compound was prepared and was isolated as a white solid (204 mg, 99%): ¹H NMR (400 MHz, CDCl₃) δ 7.94 (s, 1H), 7.67 (s, 1H), 7.11 (q, J=7.2, 6.5 Hz, 3H), 7.05-6.93 (m, 3H), 4.09 (d, J=1.9 Hz, 2H), 3.94 (s, 2H), 2.37 (s, 3H); ¹⁹F NMR (376 MHz, CDCl₃) δ −116.18; ESIMS m/z 290 ([M−H]⁻).

### 2-Chloro-N-(2-(3-fluorobenzyl)-5-methylphenyl)acetamide (C379)

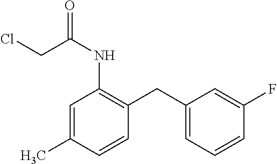 [SMILES: Cc1ccc(Cc2cccc(F)c2)c(NC(=O)CCl)c1]

The title compound was prepared and was isolated as a white solid (178 mg, 100%): ¹H NMR (400 MHz, CDCl₃) δ 7.93 (s, 1H), 7.65 (s, 1H), 7.29-7.22 (m, 1H), 7.14 (d, J=7.8 Hz, 1H), 7.03 (d, J=8.0 Hz, 1H), 6.92 (t, J=10.5 Hz, 2H), 6.83 (d, J=10.0 Hz, 1H), 4.09 (s, 2H), 3.96 (d, J=2.5 Hz, 2H), 2.37 (d, J=2.3 Hz, 3H); ¹⁹F NMR (376 MHz, CDCl₃) δ −112.71; ESIMS m/z 290 ([M−H]⁻).

### 2-Chloro-N-(2-(2-fluorobenzyl)-5-methylphenyl)acetamide (C380)

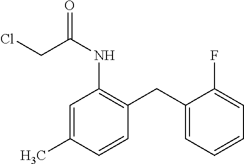 [SMILES: Cc1ccc(Cc2ccccc2F)c(NC(=O)CCl)c1]

The title compound was prepared and was isolated as a white solid (130 mg, 100%): ¹H NMR (400 MHz, CDCl₃) δ 8.06 (s, 1H), 7.63-7.59 (m, 1H), 7.25-7.17 (m, 1H), 7.13 (d, J=7.7 Hz, 1H), 7.08-6.99 (m, 4H), 4.12 (s, 2H), 3.96 (s, 2H), 2.35 (s, 3H); ¹⁹F NMR (376 MHz, CDCl₃) δ −116.98; ESIMS m/z 290 ([M−H]⁻).

### 2-Chloro-N-(5-methyl-2-(pyridin-3-ylmethyl)phenyl)acetamide (C381)

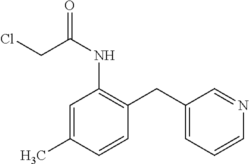 [SMILES: Cc1ccc(Cc2cccnc2)c(NC(=O)CCl)c1]

The title compound was prepared and was isolated as a tan solid (145 mg, 100%): ¹H NMR (400 MHz, CDCl₃) δ 8.50 (s, 2H), 7.96 (s, 1H), 7.61-7.56 (m, 1H), 7.43 (dd, J=7.9, 2.2 Hz, 1H), 7.23 (dd, J=7.9, 4.8 Hz, 1H), 7.12 (d, J=7.7 Hz, 1H), 7.04 (dt, J=7.8, 1.2 Hz, 1H), 4.11 (s, 2H), 3.97 (s, 2H), 2.36 (s, 3H); ESIMS m/z 275 ([M+H]⁺).

### 2-Chloro-N-(4,5-dimethyl-2-((2,2,2-trifluoroethoxy)methyl)phenyl)acetamide (C382)

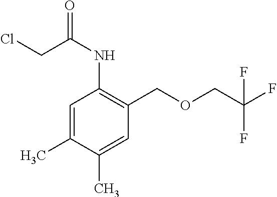 [SMILES: Cc1cc(COCC(F)(F)F)c(NC(=O)CCl)cc1C]

The title compound was prepared and was isolated as a white solid (1.075 g, 53%): ¹H NMR (400 MHz, CDCl₃) δ 9.07 (s, 1H), 7.83 (s, 1H), 6.99 (s, 1H), 4.67 (s, 2H), 4.21 (s, 2H), 3.83 (q, J=8.6 Hz, 2H), 2.28 (s, 3H), 2.23 (s, 3H); ¹⁹F NMR (376 MHz, CDCl₃) δ −73.59; ESIMS m/z 308 ([M−H]⁻).

### 2-Chloro-N-(4,5-dimethoxy-2-((2,2,2-trifluoroethoxy)methyl)phenyl)acetamide (C383)

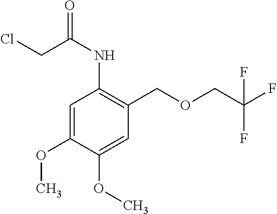 [SMILES: COc1cc(COCC(F)(F)F)c(NC(=O)CCl)cc1OC]

The title compound was prepared and was isolated as a white solid (839 mg, 95%): ¹H NMR (400 MHz, CDCl₃) δ 9.02 (s, 1H), 7.70 (s, 1H), 6.74 (s, 1H), 4.67 (s, 2H), 4.20 (s, 2H), 3.91 (s, 3H), 3.92-3.81 (m, 5H); ¹⁹F NMR (376 MHz, CDCl₃) δ −73.49; ESIMS m/z 340 ([M−H]⁻).

### 2-Chloro-N-(5-methyl-2-propoxyphenyl)acetamide (C384)

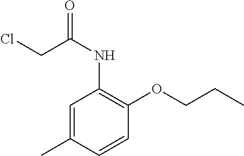 [SMILES: CCCOc1ccc(C)cc1NC(=O)CCl]

The title compound was prepared and was isolated as a pale brown solid which was used in the next step without any purification (4.0 g): ESIMS m/z 242 ([M+H]⁺).

### 2-Chloro-N-(2-(1-methoxypropyl)-5-methylphenyl)acetamide (C385)

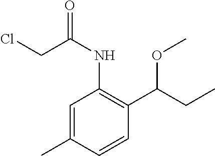 [SMILES: CCC(OC)c1ccc(C)cc1NC(=O)CCl]

The title compound was prepared and was isolated as a pale yellow semi solid which was used in the next step without any purification (1.2 g): ESIMS m/z 256 ([M+H]⁺).

### 2-Chloro-N-(2,3-dimethyl-6-((2,2,2-trifluoroethoxy)methyl)phenyl)acetamide (C386)

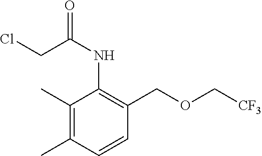 [SMILES: Cc1ccc(COCC(F)(F)F)c(NC(=O)CCl)c1C]

The title compound was prepared and was isolated as an off-white solid (2 g, 38%): ¹H NMR (400 MHz, CDCl₃) δ 8.40 (s, 1H), 7.14-7.08 (m, 2H), 4.58 (s, 2H), 4.24 (s, 2H), 3.83 (q, J=8.8 Hz, 2H), 2.32 (s, 3H), 2.14 (s, 3H).

### 2-Chloro-N-(5-methyl-2-((2,2,2-trichloroethoxy)methyl)phenyl)acetamide (C387)

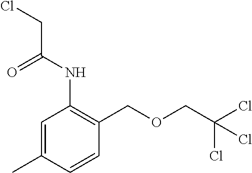 [SMILES: Cc1ccc(COCC(Cl)(Cl)Cl)c(NC(=O)CCl)c1]

The title compound was prepared and was isolated as a white solid (833 mg, 93%): ¹H NMR (400 MHz, CDCl₃) δ 9.24 (s, 1H), 7.99-7.84 (m, 1H), 7.14 (d, J=7.6 Hz, 1H), 6.98 (dd, J=8.0, 1.5 Hz, 1H), 4.83 (s, 2H), 4.20 (s, 2H), 4.05 (s, 2H), 2.39 (s, 3H); ESIMS m/z 344 ([M+H]⁺).

### 2-Chloro-N-(2-((2,2-difluoropropoxy)methyl)-5-methylphenyl)acetamide (C388)

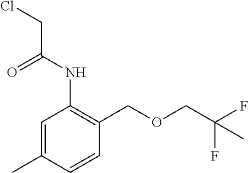 [SMILES: Cc1ccc(COCC(C)(F)F)c(NC(=O)CCl)c1]

The title compound was prepared and was isolated as a white solid (1.38 g, 78%): ¹H NMR (400 MHz, CDCl₃) δ 9.29 (s, 1H), 7.96 (d, J=1.7 Hz, 1H), 7.10 (d, J=7.7 Hz, 1H), 6.99-6.88 (m, 1H), 4.65 (s, 2H), 4.20 (s, 2H), 3.61 (t, J=12.2 Hz, 2H), 2.38 (s, 3H), 1.65 (t, J=18.8 Hz, 3H); ¹⁹F NMR (376 MHz, CDCl₃) δ −97.83; ESIMS m/z 292 ([M+H]⁺).

### 2-Chloro-N-(5-methyl-2-((3,3,3-trifluoropropoxy)methyl)phenyl)acetamide (C389)

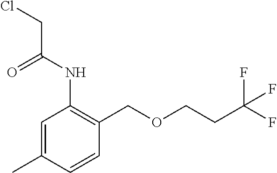 [SMILES: Cc1ccc(COCCC(F)(F)F)c(NC(=O)CCl)c1]

The title compound was prepared and was isolated as a colorless oil (1.02 g, 77%): ¹H NMR (400 MHz, CDCl₃) δ 9.37 (s, 1H), 8.01-7.92 (m, 1H), 7.10 (d, J=7.6 Hz, 1H), 6.94 (dd, J=7.5, 1.6 Hz, 1H), 4.56 (s, 2H), 4.20 (s, 2H), 3.71 (t, J=6.7 Hz, 2H), 2.46 (qt, J=10.6, 6.7 Hz, 2H), 2.37 (s, 3H); ¹⁹F NMR (376 MHz, CDCl₃) δ −64.72; ESIMS m/z 310 ([M+H]⁺).

### 2-Chloro-N-(5-methyl-2-(propoxymethyl)phenyl)acetamide (C390)

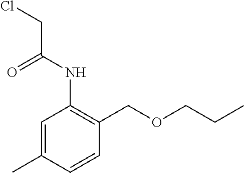 [SMILES: CCCOCc1ccc(C)cc1NC(=O)CCl]

The title compound was prepared and was isolated as a colorless oil (0.803 g, 98%): ¹H NMR (400 MHz, CDCl₃) δ 9.69 (s, 1H), 8.05-7.90 (m, 1H), 7.08 (d, J=7.6 Hz, 1H), 6.92 (dt, J=7.5, 1.3 Hz, 1H), 4.53 (s, 2H), 4.20 (s, 2H), 3.45 (t, J=6.8 Hz, 2H), 2.36 (s, 3H), 1.67 (p, J=7.2 Hz, 2H), 0.93 (t, J=7.4 Hz, 3H); ¹³C NMR (101 MHz, CDCl₃) δ 164.32, 139.23, 136.71, 129.22, 125.26, 124.49, 122.41, 72.23, 71.78, 43.09, 22.87, 21.49, 10.57.

### 2-Chloro-N-(2-((2,2-dichloroethoxy)methyl)-5-methylphenyl)acetamide (C391)

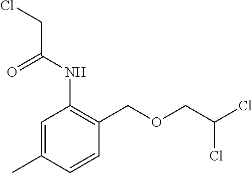 [SMILES: Cc1ccc(COCC(Cl)Cl)c(NC(=O)CCl)c1]

The title compound was prepared and was isolated as a white solid (1.04 g, 84%): ¹H NMR (400 MHz, CDCl₃) δ 9.26 (s, 1H), 7.95 (d, J=1.6 Hz, 1H), 7.11 (d, J=7.6 Hz, 1H), 6.96 (dt, J=7.7, 1.2 Hz, 1H), 5.74 (t, J=5.9 Hz, 1H), 4.68 (s, 2H), 4.20 (s, 2H), 3.86 (d, J=5.9 Hz, 2H), 2.38 (s, 3H); ESIMS m/z 311 ([M+H]⁺).

### 2-Chloro-N-(2-((2-fluoroethoxy)methyl)-5-methylphenyl)acetamide (C392)

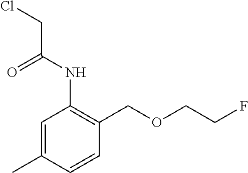 [SMILES: Cc1ccc(COCCF)c(NC(=O)CCl)c1]

The title compound was prepared and was isolated as a white solid (593 mg, 85%): ¹H NMR (400 MHz, CDCl₃) δ 9.49 (s, 1H), 7.98 (d, J=1.6 Hz, 1H), 7.10 (d, J=7.6 Hz, 1H), 6.93 (dt, J=7.4, 1.5 Hz, 1H), 4.68-4.64 (m, 1H), 4.57-4.51 (m, 1H), 4.20 (s, 2H), 3.81-3.76 (m, 1H), 3.74-3.68 (m, 1H), 2.37 (s, 3H).

### 2-Chloro-N-(2-((2,2,2-trifluoroethoxy)methyl)phenyl)acetamide (C393)

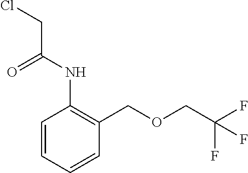 [SMILES: O=C(CCl)Nc1ccccc1COCC(F)(F)F]

The title compound was prepared and was isolated as a white solid (4.84 g, 94%): ¹H NMR (400 MHz, CDCl₃) δ 9.24 (s, 1H), 8.17-8.05 (m, 1H), 7.42 (td, J=7.8, 1.7 Hz, 1H), 7.25-7.09 (m, 2H), 4.74 (s, 2H), 4.21 (s, 2H), 3.87 (q, J=8.5 Hz, 2H); ¹⁹F NMR (376 MHz, CDCl₃) δ −73.55; ESIMS m/z 282 ([M+H]⁺).

### 2-Chloro-N-(5-ethyl-2-((2,2,2-trifluoroethoxy)methyl)phenyl)acetamide (C394)

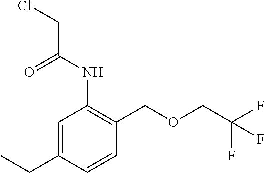 [SMILES: CCc1ccc(COCC(F)(F)F)c(NC(=O)CCl)c1]

The title compound was prepared and was isolated as a white solid (713 mg, 95%): ¹H NMR (400 MHz, CDCl₃) δ 9.18 (s, 1H), 7.97 (d, J=1.5 Hz, 1H), 7.15 (d, J=7.7 Hz, 1H), 7.00 (d, J=7.2 Hz, 1H), 4.71 (s, 2H), 4.20 (s, 2H), 3.85 (q, J=8.5 Hz, 2H), 2.68 (q, J=7.6 Hz, 2H), 1.25 (t, J=7.6 Hz, 3H); ¹⁹F NMR (376 MHz, CDCl₃) δ −73.55; ESIMS m/z 310 ([M+H]⁺).

### 2-Chloro-N-(4-methyl-2-((2,2,2-trifluoroethoxy)methyl)phenyl)acetamide (C395)

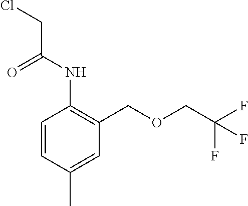 [SMILES: Cc1ccc(NC(=O)CCl)c(COCC(F)(F)F)c1]

The title compound was prepared and was isolated a white solid (2.1 g, 95%): ¹H NMR (400 MHz, CDCl₃) δ 9.11 (s, 1H), 7.95 (d, J=8.3 Hz, 1H), 7.21 (dd, J=8.2, 2.1 Hz, 1H), 7.05 (d, J=2.1 Hz, 1H), 4.69 (s, 2H), 4.19 (s, 2H), 3.85 (q, J=8.5 Hz, 2H), 2.33 (s, 3H); ¹⁹F NMR (376 MHz, CDCl₃) δ −73.58.

### 2-Chloro-N-(2-((2,2-difluoroethoxy)methyl)-5-methylphenyl)acetamide (C396)

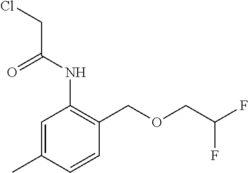 [SMILES: Cc1ccc(COCC(F)F)c(NC(=O)CCl)c1]

The title compound was prepared and was isolated as an off-white solid (10.3 g, 99%): ¹H NMR (400 MHz, CDCl₃) δ 9.29 (s, 1H), 7.96 (d, J=1.7 Hz, 1H), 7.11 (d, J=7.6 Hz, 1H), 7.01-6.88 (m, 1H), 5.91 (tt, J=55.2, 4.0 Hz, 1H), 4.65 (s, 2H), 4.20 (s, 2H), 3.70 (td, J=13.9, 4.0 Hz, 2H), 2.38 (s, 3H); ¹⁹F NMR (376 MHz, CDCl₃) δ −124.66.

### 2-Chloro-N-(2-((2-chloro-2,2-difluoroethoxy)methyl)-5-methylphenyl)acetamide (C397)

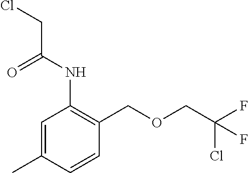 [SMILES: Cc1ccc(COCC(F)(F)Cl)c(NC(=O)CCl)c1]

The title compound was prepared and was isolated as an off-white solid (841 mg, 95%): ¹H NMR (400 MHz, CDCl₃) δ 9.17 (s, 1H), 7.99-7.85 (m, 1H), 7.12 (d, J=7.7 Hz, 1H), 6.97 (dt, J=7.6, 1.2 Hz, 1H), 4.73 (s, 2H), 4.20 (s, 2H), 3.92 (t, J=11.0 Hz, 2H), 2.38 (s, 3H); ¹⁹F NMR (376 MHz, CDCl₃) δ −61.08.

### 2-Chloro-N-(3-methyl-2-((2,2,2-trifluoroethoxy)methyl)phenyl)acetamide (C398)

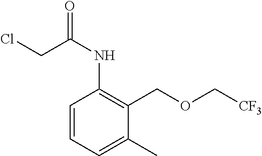 [SMILES: Cc1cccc(NC(=O)CCl)c1COCC(F)(F)F]

The title compound was prepared and was isolated as an off-white solid (4.5 g, 84%): ¹H NMR (300 MHz, DMSO-d₆) δ 9.80 (s, 1H), 7.32-7.16 (m, 2H), 7.16-7.06 (m, 1H), 4.65 (s, 2H), 4.30 (s, 2H), 4.01 (q, J=9.3 Hz, 2H), 2.36 (s, 3H).

### 2-Chloro-N-(5-methoxy-2-((2,2,2-trifluoroethoxy)methyl)phenyl)acetamide (C399)

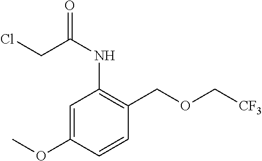 [SMILES: COc1ccc(COCC(F)(F)F)c(NC(=O)CCl)c1]

The title compound was prepared and was isolated as an off-white solid (6.5 g, 98%): ¹H NMR (400 MHz, CDCl₃) δ 9.23 (s, 1H), 7.81 (d, J=2.8 Hz, 1H), 7.13 (d, J=8.4 Hz, 1H), 6.68 (dd, J=2.8, 8.4 Hz, 1H), 4.68 (s, 2H), 4.20 (s, 2H), 3.88-3.74 (m, 5H).

### 2-Chloro-N-(5-fluoro-2-((2,2,2-trifluoroethoxy)methyl)phenyl)acetamide (C400

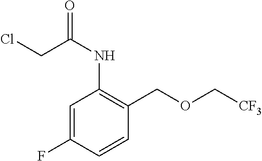 [SMILES: O=C(CCl)Nc1cc(F)ccc1COCC(F)(F)F]

The title compound was prepared and was isolated as an off-white solid (4.6 g, 87%): ¹H NMR (400 MHz, CDCl₃) δ 9.34 (s, 1H), 8.02 (dd, J=2.4, 10.8 Hz, 1H), 7.22-7.13 (m, 1H), 6.89-6.80 (m, 1H), 4.72 (s, 2H), 4.20 (s, 2H), 3.86 (q, J=8.4 Hz, 2H).

### 2-Chloro-N-(5-cyano-2-(1-methoxyethyl)phenyl)acetamide (C401)

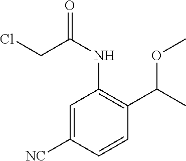 [SMILES: COC(C)c1ccc(C#N)cc1NC(=O)CCl]

The title compound was prepared and was isolated as a pale yellow semi-solid (0.9 g, used without purification); ESIMS m/z 251 ([M−H]⁻).

### 2-Chloro-N-(5-(dimethylamino)-2-(methoxymethyl)phenyl)acetamide (C402)

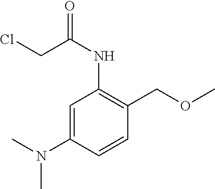 [SMILES: COCc1ccc(N(C)C)cc1NC(=O)CCl]

The title compound was prepared and was isolated as pale yellow solid (2.5 g, used without purification): ESIMS m/z 257 ([M+H]⁺).

The following compounds were prepared in accordance to the procedure in Example 46:

### 4,5-Dimethyl-2-((2,2,2-trifluoroethoxy)methyl)aniline (C403)

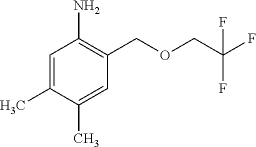 [SMILES: Cc1cc(N)c(COCC(F)(F)F)cc1C]

The title compound was prepared with palladium hydroxide on carbon in EtOAc and was isolated as a white solid (1.50 g, 96%): ¹H NMR (400 MHz, CDCl₃) δ 6.80 (s, 1H), 6.53 (s, 1H), 4.63 (s, 2H), 3.90 (s, 2H), 3.75 (q, J=8.8 Hz, 2H), 2.18 (s, 3H), 2.14 (s, 3H); ¹⁹F NMR (376 MHz, CDCl₃) δ −73.81.

### 4,5-Dimethoxy-2-((2,2,2-trifluoroethoxy)methyl)aniline (C404)

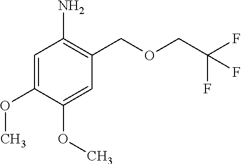 [SMILES: COc1cc(N)c(COCC(F)(F)F)cc1OC]

The title compound was prepared with palladium hydroxide on carbon in EtOAc and was isolated as a colorless oil (681 mg, 96%): ¹H NMR (400 MHz, CDCl₃) δ 6.63 (s, 1H), 6.31 (s, 1H), 4.62 (s, 2H), 3.94-3.71 (m, 10H); ¹⁹F NMR (376 MHz, CDCl₃) δ −73.72.

### 2-(1-Methoxypropyl)-5-methylaniline (C405)

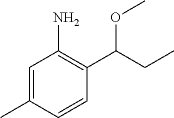

The title compound was prepared with 20% palladium hydroxide on carbon in EtOAc and was isolated as a pale brown liquid; ¹H NMR (400 MHz, CDCl₃) δ 6.83 (d, J=8.4 Hz, 1H), 6.51-6.49 (m, 1H), 6.45 (s, 1H), 4.21 (br s, 2H), 4.03 (t, J=7.2 Hz, 1H), 3.25 (s, 3H), 2.25 (s, 3H), 1.97-1.79 (m, 2H), 0.88 (t, J=7.2 Hz, 3H).

### 2-((2,2-Difluoropropoxy)methyl)-5-methylaniline (C406)

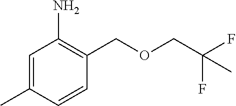

The title compound was prepared with 20% palladium hydroxide on carbon in EtOAc and was isolated as a yellow oil (1.28 g, 99%): ¹H NMR (400 MHz, CDCl₃) δ 6.98-6.85 (m, 1H), 6.53 (d, J=6.5 Hz, 2H), 4.59 (s, 2H), 3.72 (t, J=12.4 Hz, 1H), 3.54 (t, J=12.4 Hz, 2H), 2.26 (s, 3H), 1.62 (t, J=18.8 Hz, 3H); ¹⁹F NMR (376 MHz, CDCl₃) δ −97.80; ESIMS m/z 216 ([M+H]⁺).

### 5-Methyl-2-((3,3,3-trifluoropropoxy)methyl)aniline (C407)

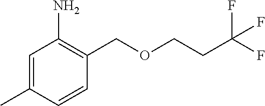 [SMILES: Cc1ccc(COCCC(F)(F)F)c(N)c1]

The title compound was prepared with 20% palladium hydroxide on carbon in EtOAc and was isolated as a white solid (970 mg, 100%): ¹H NMR (400 MHz, CDCl₃) δ 6.99-6.89 (m, 1H), 6.53 (d, J=6.2 Hz, 2H), 4.51 (s, 2H), 4.20-3.76 (m, 2H), 3.63 (t, J=6.4 Hz, 2H), 2.40 (qt, J=10.8, 6.4 Hz, 2H), 2.26 (s, 3H); ¹⁹F NMR (376 MHz, CDCl₃) δ −64.64; ESIMS m/z 234 ([M+H]⁺).

### 5-Methyl-2-(propoxymethyl)aniline (C408)

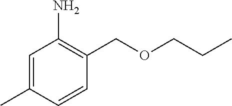

The title compound was prepared with 20% palladium hydroxide on carbon in EtOAc and was isolated as a colorless oil (637 mg, 89%): ¹H NMR (400 MHz, CDCl₃) δ 6.98-6.88 (m, 1H), 6.52 (d, J=6.8 Hz, 2H), 4.48 (s, 2H), 4.19-3.73 (m, 2H), 3.38 (t, J=6.7 Hz, 2H), 2.25 (s, 3H), 1.60 (h, J=7.2 Hz, 2H), 0.91 (t, J=7.4 Hz, 3H); ESIMS m/z 180 ([M+H]⁺).

### 2-((2,2-Dichloroethoxy)methyl)-5-methylaniline_(C409)

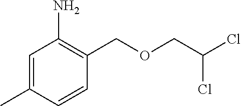

The title compound was prepared with 20% palladium hydroxide on carbon in EtOAc and was isolated as a pink solid (910 mg, 93%): ¹H NMR (400 MHz, CDCl₃) δ 6.94 (d, J=7.9 Hz, 1H), 6.55-6.50 (m, 3H), 5.72 (t, J=5.9 Hz, 1H), 4.62 (s, 3H), 4.27-3.94 (m, 2H), 3.80 (d, J=5.9 Hz, 3H); ESIMS m/z 234 ([M+H]⁺).

### 2-((2-Fluoroethoxy)methyl)-5-methylaniline (C410)

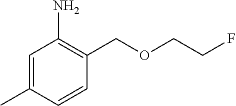

The title compound was prepared with 20% palladium hydroxide on carbon in EtOAc and was isolated as a colorless oil (482 mg, 97%): ¹H NMR (400 MHz, CDCl₃) δ 7.00-6.88 (m, 1H), 6.57-6.43 (m, 2H), 4.65-4.59 (m, 1H), 4.57 (s, 2H), 4.52-4.47 (m, 1H), 3.98 (s, br, 2H), 3.72-3.66 (m, 1H), 3.65-3.58 (m, 1H), 2.26 (s, 3H); ESIMS m/z 184 ([M+H]⁺).

### 2-((2,2,2-Trifluoroethoxy)methyl)aniline (C411)

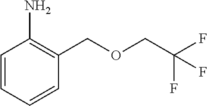 [SMILES: Nc1ccccc1COCC(F)(F)F]

The title compound was prepared with 20% palladium hydroxide on carbon in EtOAc and was isolated as a colorless oil (3.67 g, 89%): ¹H NMR (400 MHz, CDCl₃) δ 7.23-7.13 (m, 1H), 7.06 (d, J=7.4 Hz, 1H), 6.81-6.65 (m, 2H), 4.69 (s, 2H), 4.11 (d, J=6.1 Hz, 2H), 3.77 (qd, J=8.7, 1.1 Hz, 2H); ¹⁹F NMR (376 MHz, CDCl₃) δ −74.12; ESIMS m/z 206 ([M+H]⁺).

### 5-Ethyl-2-((2,2,2-trifluoroethoxy)methyl)aniline (C412)

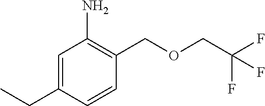 [SMILES: CCc1ccc(COCC(F)(F)F)c(N)c1]

The title compound was prepared with 20% palladium hydroxide on carbon in EtOAc and was isolated as a yellow oil (587 mg, 96%): ¹H NMR (400 MHz, CDCl₃) δ 6.97 (d, J=7.4 Hz, 1H), 6.57 (d, J=8.4 Hz, 2H), 4.66 (s, 2H), 4.06 (s, 2H), 3.76 (q, J=8.7 Hz, 2H), 2.56 (q, J=7.6 Hz, 2H), 1.21 (t, J=7.6 Hz, 3H); ¹⁹F NMR (376 MHz, CDCl₃) δ −73.86; ESIMS m/z 234 ([M+H]⁺).

### 2-((2-Chloro-2,2-difluoroethoxy)methyl)-5-methylaniline (C413)

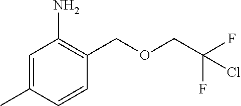

The title compound was prepared with 20% palladium hydroxide on carbon in EtOAc and was isolated as red crystals (600 mg, 80%): ¹H NMR (400 MHz, CDCl₃) δ 6.99-6.88 (m, 1H), 6.60-6.48 (m, 2H), 4.68 (s, 2H), 4.07 (s, 2H), 3.84 (t, J=11.1 Hz, 2H), 2.27 (s, 3H); ¹⁹F NMR (376 MHz, CDCl₃) δ −60.99.

### 2-((2,2-Difluoroethoxy)methyl)-5-methylaniline (C414)

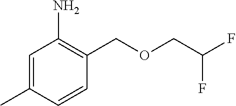

The title compound was prepared with 20% palladium hydroxide on carbon in EtOAc and was isolated as light yellow crystals (6.9 g, 98%): ¹H NMR (400 MHz, CDCl₃) δ 6.99-6.87 (m, 1H), 6.59-6.47 (m, 2H), 5.85 (tt, J=55.4, 4.0 Hz, 1H), 4.59 (s, 2H), 4.09 (d, J=7.6 Hz, 2H), 3.61 (td, J=14.1, 4.1 Hz, 2H), 2.26 (s, 3H); ¹⁹F NMR (376 MHz, CDCl₃) δ −125.1.

### 4-Methyl-2-((2,2,2-trifluoroethoxy)methyl)aniline (C415)

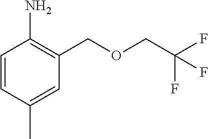

The title compound was prepared with 20% palladium hydroxide on carbon in EtOAc and was isolated as a light yellow oil (1.6 g, 94%): ¹H NMR (400 MHz, CDCl₃) δ 7.01-6.95 (m, 1H), 6.87 (d, J=2.1 Hz, 1H), 6.62 (d, J=8.0 Hz, 1H), 4.65 (s, 2H), 3.96 (s, 2H), 3.77 (q, J=8.7 Hz, 2H), 2.24 (s, 3H); ¹⁹F NMR (376 MHz, CDCl₃) δ −73.82.

### 3-Methyl-2-((2,2,2-trifluoroethoxy)methyl)aniline (C416)

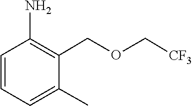

The title compound was prepared with 20% palladium hydroxide on carbon and was isolated as a pale brown liquid (4 g, 88%): ¹H NMR (300 MHz, DMSO-d₆) δ 6.98-6.84 (m, 1H), 6.51 (d, J=8.1 Hz, 1H), 6.39 (d, J=7.2 Hz, 1H), 4.98 (s, 2H), 4.60 (s, 2H), 4.03 (q, J=9.6 Hz, 2H), 2.22 (s, 3H).

### 5-Methoxy-2-((2,2,2-trifluoroethoxy)methyl)aniline (C417)

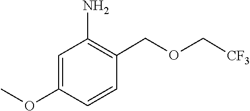 [SMILES: COc1ccc(COCC(F)(F)F)c(N)c1]

The title compound was prepared with 20% palladium hydroxide on carbon and was isolated as a pale brown liquid (5.0 g, 94%): ¹H NMR (400 MHz, CDCl₃) δ 6.97 (d, J=8.0 Hz, 1H), 6.34-6.22 (m, 2H), 4.63 (s, 2H), 4.10 (s, 2H), 3.78-3.70 (m, 5H).

### 5-Fluoro-2-((2,2,2-trifluoroethoxy)methyl)aniline (C418)

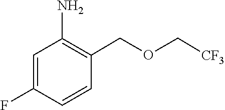 [SMILES: Nc1cc(F)ccc1COCC(F)(F)F]

The title compound was prepared with 20% palladium hydroxide on carbon and was isolated as a pale brown liquid (4.0 g, 95%): ¹H NMR (400 MHz, CDCl₃) δ 7.01-6.94 (m, 1H), 6.43-6.30 (m, 2H), 4.64 (s, 2H), 4.22 (s, 2H), 3.76 (q, J=8.8 Hz, 2H).

### 4-(Methoxymethyl)-N¹,N¹-dimethylbenzene-1,3-diamine (C419)

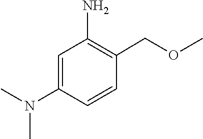

The title compound was prepared and was isolated as a brown liquid (3.4 g, 79%): ESIMS m/z 181 ([M+H]⁺).

The following compounds were prepared in accordance to the procedure in Example 48:

### 2-Benzyl-5-methylaniline (C420)

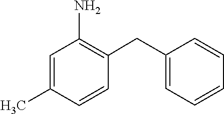

The title compound was prepared and was isolated as a pale yellow oil (0.462 g, 96%): ¹H NMR (400 MHz, CDCl₃) δ 7.32-7.24 (m, 2H), 7.22-7.16 (m, 3H), 6.94 (d, J=7.5 Hz, 1H), 6.59 (dd, J=7.4, 1.6 Hz, 1H), 6.51 (d, J=1.7 Hz, 1H), 3.87 (s, 2H), 3.47 (s, 2H), 2.26 (s, 3H); ESIMS m/z 198 ([M+H]⁺).

### 5-Methyl-2-(3-(trifluoromethyl)benzyl)aniline (C421)

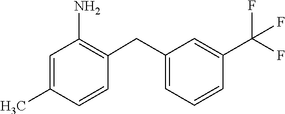 [SMILES: Cc1ccc(Cc2cccc(C(F)(F)F)c2)c(N)c1]

The title compound was prepared and was isolated as a pale yellow oil (440 mg, 82%): ¹H NMR (400 MHz, CDCl₃) δ 7.48-7.45 (m, 1H), 7.38 (dd, J=15.5, 7.7 Hz, 2H), 7.35-7.30 (m, 1H), 6.91 (d, J=7.6 Hz, 1H), 6.63-6.59 (m, 1H), 6.54 (d, J=1.7 Hz, 1H), 3.91 (s, 2H), 3.47-3.40 (m, 2H), 2.27 (s, 3H); ¹⁹F NMR (376 MHz, CDCl₃) δ −62.53; EIMS m/z 265.

### 2-(Furan-2-yl)-5-methylaniline (C421)

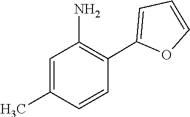

The title compound was prepared and was isolated as a pale yellow oil (0.370 g, 98%): ¹H NMR (400 MHz, CDCl₃) δ 7.47 (dd, J=1.9, 0.8 Hz, 1H), 7.35 (d, J=7.9 Hz, 1H), 6.61 (dd, J=8.1, 1.7 Hz, 1H), 6.57 (d, J=1.7 Hz, 1H), 6.51 (dd, J=3.4, 0.8 Hz, 1H), 6.49 (dd, J=3.4, 1.8 Hz, 1H), 4.25 (s, 2H), 2.28 (s, 3H); ESIMS m/z 174 ([M+H]⁺).

### 2-(4-Fluorobenzyl)-5-methylaniline (C422)

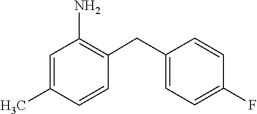 [SMILES: Cc1ccc(Cc2ccc(F)cc2)c(N)c1]

The title compound was prepared and was isolated as a red oil (164 mg, 58%): ¹H NMR (400 MHz, CDCl₃) δ 7.26 (d, J=1.1 Hz, 1H), 7.13 (dd, J=8.2, 5.4 Hz, 2H), 7.00-6.89 (m, 2H), 6.59 (d, J=7.6 Hz, 1H), 6.52 (s, 1H), 3.83 (s, 2H), 3.43 (s, 2H), 2.27 (s, 3H); ¹⁹F NMR (376 MHz, CDCl₃) δ −117.03; ESIMS m/z 216 ([M+H]⁺).

### 2-(3-Fluorobenzyl)-5-methylaniline (C423)

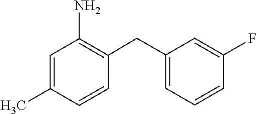 [SMILES: Cc1ccc(Cc2cccc(F)c2)c(N)c1]

The title compound was prepared and was isolated as a red oil (140 mg, 61%): ¹H NMR (400 MHz, CDCl₃) δ 7.28-7.20 (m, 1H), 6.98 (d, J=7.7 Hz, 1H), 6.96-6.83 (m, 3H), 6.60 (d, J=7.6 Hz, 1H), 6.52 (s, 1H), 3.86 (s, 2H), 3.43 (s, 2H), 2.27 (s, 3H); ¹⁹F NMR (376 MHz, CDCl₃) δ −113.18; ESIMS m/z 216 ([M+H]⁺).

### 2-(2-Fluorobenzyl)-5-methylaniline (C424)

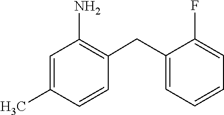 [SMILES: Cc1ccc(Cc2ccccc2F)c(N)c1]

The title compound was prepared and was isolated as a white solid (106 mg, 30%): ¹H NMR (400 MHz, CDCl₃) δ 7.19 (tdd, J=7.4, 5.3, 2.1 Hz, 1H), 7.09-6.99 (m, 3H), 6.93 (d, J=7.6 Hz, 1H), 6.58 (dd, J=7.7, 1.7 Hz, 1H), 6.52 (d, J=1.7 Hz, 1H), 3.85 (s, 2H), 3.54 (s, 2H), 2.26 (s, 3H); ¹⁹F NMR (376 MHz, CDCl₃) δ −117.65; EIMS m/z 215.

### 5-Methyl-2-(pyridin-3-ylmethyl)aniline (C425)

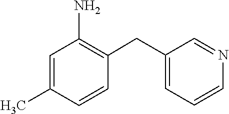

The title compound was prepared and was isolated as an orange foam (108 mg, 97%): ¹H NMR (400 MHz, CDCl₃) δ 8.52 (d, J=2.3 Hz, 1H), 8.47 (dd, J=4.9, 1.7 Hz, 1H), 7.45 (dt, J=7.9, 2.0 Hz, 1H), 7.24-7.16 (m, 1H), 6.90 (d, J=7.6 Hz, 1H), 6.59 (dd, J=7.7, 1.7 Hz, 1H), 6.53 (d, J=1.8 Hz, 1H), 3.85 (s, 2H), 3.40 (s, 2H), 2.27 (s, 3H); ¹³C NMR (101 MHz, CDCl₃) δ 149.46, 147.28, 143.71, 137.37, 135.48, 134.68, 130.11, 123.05, 120.31, 119.30, 116.31, 34.08, 20.58; EIMS m/z 198.

### 5-Methyl-2-propoxyaniline (C426)

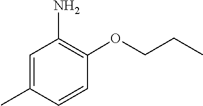

The title compound was prepared and was isolated as a yellow liquid (3.4 g, used without purification); ESIMS m/z 166 ([M+H]⁺).

### 2,3-Dimethyl-6-((2,2,2-trifluoroethoxy)methyl)aniline (C427)

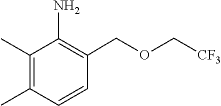 [SMILES: Cc1ccc(COCC(F)(F)F)c(N)c1C]

The title compound was prepared and was isolated as a pale yellow liquid which was used in the next step without purification (4.0 g, 87%): ESIMS m/z 234 ([M+H]⁺).

### 5-Methyl-2-((2,2,2-trichloroethoxy)methyl)aniline (C428)

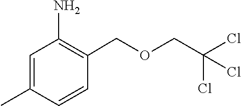

The title compound was prepared and was isolated as a white solid (685 mg, 76%): ¹H NMR (400 MHz, CDCl₃) δ 6.96 (d, J=8.0 Hz, 1H), 6.55 (d, J=6.0 Hz, 2H), 4.78 (s, 2H), 4.15 (s, 2H), 4.01 (s, 2H), 2.27 (s, 3H); ESIMS m/z 268 ([M+H]⁺).

The following compound was prepared in accordance to the procedure in Example 64.

### 1-(1-Methoxypropyl)-4-methyl-2-nitrobenzene (C429)

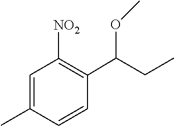

The title compound was prepared and was isolated as a yellow liquid (2.5 g, 38%): ¹H NMR (400 MHz, CDCl₃) δ 7.12 (d, J=0.8 Hz, 1H), 7.56 (d, J=8.0 Hz, 1H), 7.45-7.25 (m, 1H), 4.65-4.62 (m, 1H), 3.20 (s, 3H), 2.42 (s, 3H), 1.79-1.67 (m, 2H), 0.99 (t, J=7.2 Hz, 3H).

### 4-(Methoxymethyl)-N,N-dimethyl-3-nitroaniline (C430)

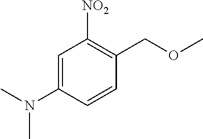

The title compound was prepared and was isolated as a yellow solid (4.5 g, 52%): ESIMS m/z 211 ([M+H]⁺).

### 4-(1-Methoxyethyl)-3-nitrobenzonitrile (C431)

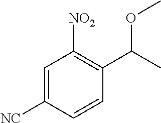

The title compound was prepared and was isolated as a pale yellow liquid (1.0 g, 33%): ESIMS m/z 207 ([M+H]⁺).

The following compound was prepared in accordance to the procedure in Example 73.

### 1,2-Dimethyl-4-nitro-5-((2,2,2-trifluoroethoxy)methyl)benzene (C432)

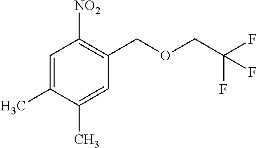 [SMILES: Cc1cc(COCC(F)(F)F)c([N+](=O)[O-])cc1C]

The title compound was prepared using 2,2,2-trifluoroethyl trifluoromethanesulfonate and was isolated as a white solid (1.75 g, 60%): ¹H NMR (400 MHz, CDCl₃) δ 7.92 (s, 1H), 7.50 (s, 1H), 5.03 (s, 2H), 3.98 (q, J=8.6 Hz, 2H), 2.37 (s, 3H), 2.34 (s, 3H); ¹⁹F NMR (376 MHz, CDCl₃) δ −74.03.

### 1,2-Dimethoxy-4-nitro-5-((2,2,2-trifluoroethoxy)methyl)benzene (C433)

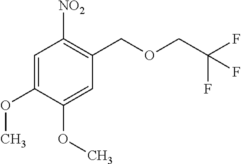 [SMILES: COc1cc(COCC(F)(F)F)c([N+](=O)[O-])cc1OC]

The title compound was prepared using 2,2,2-trifluoroethyl trifluoromethanesulfonate and was isolated as a yellow solid (781 mg, 28%): ¹H NMR (400 MHz, CDCl₃) δ 7.73 (s, 1H), 7.28 (s, 1H), 5.10 (s, 2H), 4.02 (q, J=8.6 Hz, 2H), 4.00 (s, 3H), 3.96 (s, 3H); ¹⁹F NMR (376 MHz, CDCl₃) δ −74.08.

### 2-Nitro-1-((2,2,2-trifluoroethoxy)methyl)-4-vinylbenzene (C434)

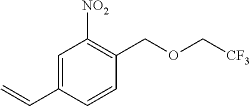 [SMILES: C=Cc1ccc(COCC(F)(F)F)c([N+](=O)[O-])c1]

The title compound was prepared and was isolated as a yellow oil (687 mg, 30%): ¹H NMR (400 MHz, CDCl₃) δ 8.13 (d, J=1.8 Hz, 1H), 7.80-7.63 (m, 2H), 6.75 (dd, J=17.6, 10.9 Hz, 1H), 5.89 (d, J=17.5 Hz, 1H), 5.45 (d, J=10.9 Hz, 1H), 5.07 (s, 2H), 4.00 (q, J=8.6 Hz, 2H); ¹⁹F NMR (376 MHz, CDCl₃) δ −74.08; ESIMS m/z 262 ([M+H]⁺).

### 1,2-Dimethyl-3-nitro-4-((2,2,2-trifluoroethoxy)methyl)benzene (C435)

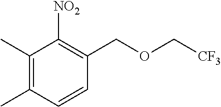 [SMILES: Cc1ccc(COCC(F)(F)F)c([N+](=O)[O-])c1C]

The title compound was prepared using 2,2,2-trifluoroethyl trifluoromethanesulfonate and was isolated as a pale yellow liquid which was used in next step without any analysis (5.2 g, 55%).

### 4-Methyl-1-nitro-2-((2,2,2-trifluoroethoxy)methyl)benzene (C436)

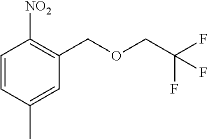 [SMILES: Cc1ccc([N+](=O)[O-])c(COCC(F)(F)F)c1]

The title compound was prepared using 2,2,2-trifluoroethyl trifluoromethanesulfonate in THF and was isolated as an off-white solid (1.9 g, 64%): ¹H NMR (400 MHz, CDCl₃) δ 8.04 (d, J=8.4 Hz, 1H), 7.58 (dd, J=2.0, 1.0 Hz, 1H), 7.25 (d, J=2.0 Hz, 1H), 5.07 (s, 2H), 4.01 (q, J=8.6 Hz, 2H), 2.48 (s, 3H); ¹⁹F NMR (376 MHz, CDCl₃) δ−74.03.

The following compounds were prepared in accordance to the procedure in Example 76:

### (4,5-Dimethyl-2-nitrophenyl)methanol (C437)

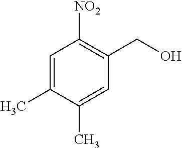

The title compound was prepared and was isolated as a brown solid (2.6 g, 88%): ¹H NMR (400 MHz, CDCl₃) δ 7.91 (s, 1H), 7.42 (s, 1H), 4.89 (s, 2H), 2.62 (s, 1H), 2.36 (s, 3H), 2.34 (s, 3H); ¹³C NMR (101 MHz, CDCl₃) δ 145.44, 144.37, 137.53, 134.11, 131.49, 126.07, 62.71, 19.92, 19.36.

### (2-Methyl-6-nitrophenyl)methanol (C438)

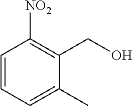

The title compound was prepared and was isolated as a pale yellow solid (9.0 g, 97%): ¹H NMR (300 MHz, DMSO-d₆) δ 7.60 (d, J=8.1 Hz, 1H), 7.48 (d, J=7.2 Hz, 1H), 7.46-7.28 (m, 1H), 5.20 (t, J=5.1 Hz, 1H), 4.62 (d, J=5.4 Hz, 2H), 2.43 (s, 3H).

### (4-Methoxy-2-nitrophenyl)methanol (C439)

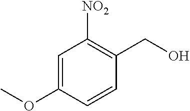

The title compound was prepared and was isolated as a pale yellow solid (9.0 g, 97%): ¹H NMR (300 MHz, DMSO-d₆) δ 7.70 (d, J=8.4 Hz, 1H), 7.54 (d, J=3.0 Hz, 1H), 7.34 (dd, J=2.4, 9.0 Hz, 1H), 5.43 (t, J=5.4 Hz, 1H), 4.72 (d, J=5.7 Hz, 2H), 3.84 (s, 3H).

### (4-Fluoro-2-nitrophenyl)methanol (C440)

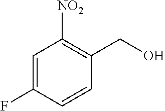 [SMILES: O=[N+]([O-])c1cc(F)ccc1CO]

The title compound was prepared and was isolated as an off-white solid (8.5 g, 92%): ¹H NMR (300 MHz, DMSO-d₆) δ 7.95 (dd, J=2.4, 8.7 Hz, 1H), 7.80-7.82 (m, 1H), 7.72-7.60 (m, 1H), 5.59 (t, J=5.4 Hz, 1H), 4.78 (d, J=5.4 Hz, 2H). (q, J=8.5 Hz, 2H); ¹⁹F NMR (376 MHz, CDCl₃) δ −70.62; EIMS m/z 255.

### Example 82: Preparation of 4-bromo-2-(2,2,2-trifluoroethoxy)pyridine (C441) and 4-bromo-1-(2,2,2-trifluoroethyl)pyridin-2(1H)-one (C442)

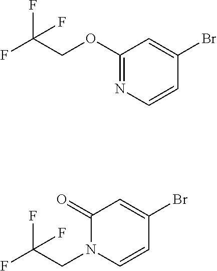 [SMILES: FC(F)(F)COc1cc(Br)ccn1 ; O=c1cc(Br)ccn1CC(F)(F)F]

To a suspension of 4-bromopyridin-2(1H)-one (3.0 g, 17.24 mmol) and potassium carbonate (3.10 g, 22.41 mmol) in dry DMF (20 mL) was added slowly a solution of 2,2,2-trifluoroethyl trifluoromethanesulfonate (4.00 g, 17.24 mmol) in dry DMF (5 mL). The reaction mixture was stirred overnight at room temperature. The reaction mixture was diluted with water and extracted with EtOAc. The organic layer was washed with 1:1 water/brine three times, passed through a phase separator, and then concentrated. Purification of the residue by silica gel column chromatography eluting with 0-60% EtOAc-hexanes gave the title compounds. 4-Bromo-2-(2,2,2-trifluoroethoxy)pyridine was isolated as a colorless oil (1.4 g, 30%): ¹H NMR (400 MHz, CDCl₃) δ 7.97 (d, J=5.5 Hz, 1H), 7.13 (dd, J=5.5, 1.6 Hz, 1H), 7.08 (d, J=1.6 Hz, 1H), 4.75 (q, J=8.5 Hz, 2H); ¹⁹F NMR (376 MHz, CDCl₃) δ −73.86; EIMS m/z 255. 4-Bromo-1-(2,2,2-trifluoroethyl)pyridin-2(1H)-one was isolated as a white solid (2.1 g, 48%): ¹H NMR (400 MHz, CDCl₃) δ 7.13 (d, J=7.4 Hz, 1H), 6.89 (d, J=2.1 Hz, 1H), 6.39 (dd, J=7.4, 2.1 Hz, 1H), 4.57 (q, J=8.5 Hz, 2H); ¹⁹F NMR (376 MHz, CDCl₃) δ −70.62; EIMS m/z 255.

The following compound was prepared in accordance to the procedure in Example 82:

### 5-Bromo-2-(2,2,2-trifluoroethoxy)pyridine (C443)

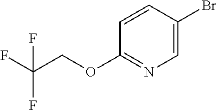 [SMILES: FC(F)(F)COc1ccc(Br)cn1]

The title compound was prepared and was isolated as a colorless oil (2.6 g, 44%): ¹H NMR (400 MHz, CDCl₃) δ 8.21-8.16 (m, 1H), 7.71 (dd, J=8.8, 2.5 Hz, 1H), 6.77 (dd, J=8.7, 0.7 Hz, 1H), 4.72 (q, J=8.5 Hz, 2H); ¹⁹F NMR (376 MHz, CDCl₃) δ−73.94.

### Example 83: Preparation of 3-bromo-1-(4-(methoxymethoxy)phenyl)-1H-1,2,4-triazole (C444)

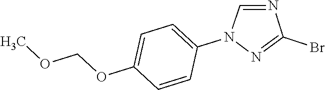 [SMILES: COCOc1ccc(-n2cnc(Br)n2)cc1]

1-lodo-4-(methoxymethoxy)benzene (3.09 g, 11.70 mmol) was combined with bis(((Z)-4-oxopent-2-en-2-yl)oxy)copper (0.613 g, 2.340 mmol) and cesium carbonate (7.63 g, 23.40 mmol) in DMF (46.8 mL) and the mixture was degassed under vacuum. Pentane-2,4-dione (0.478 mL, 4.68 mmol) and 3-bromo-1H-1,2,4-triazole (2.078 g, 14.04 mmol) were added quickly. The deep blue solution took on a green color at this point and became homogeneous. The mixture was heated to 100° C. for 15 h, was cooled to room temperature, and was poured into water. The reaction mixture was diluted with brine and acetone and filtered through diatoaceous earth. The layers were allowed to separate, and the aqueous layer was further extracted with acetone. The combined organic layers were dried over Na₂SO₄ and concentrated. Purification by silica gel chromatography eluting with 0-25% acetone-hexanes provided the title compound as a clear yellow oil (2.75 g, 83%): ¹H NMR (400 MHz, CDCl₃) δ 8.33 (s, 1H), 7.56-7.50 (m, 2H), 7.19-7.13 (m, 2H), 5.22 (s, 2H), 3.50 (s, 3H); EIMS m/z 285.

The following compound was prepared in accordance to the procedure in Example 83:

### 2-(4-(3-Bromo-1H-1,2,4-triazol-1-yl)phenyl)acetonitrile (C445)

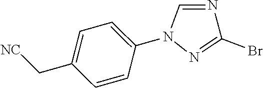 [SMILES: N#CCc1ccc(-n2cnc(Br)n2)cc1]

The title compound was prepared and was isolated as an off-white solid (0.600 g, 22%): ¹H NMR (400 MHz, Acetone-d₆) δ 9.07 (s, 1H), 7.94-7.86 (m, 2H), 7.67-7.59 (m, 2H), 4.09 (s, 2H); EIMS m/z 262.

### Example 84: Preparation of 3-bromo-1-(4-((trifluoromethyl)thio)phenyl)-1H-1,2,4-triazole (C446)

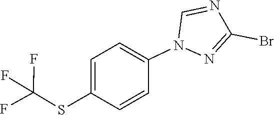 [SMILES: FC(F)(F)Sc1ccc(-n2cnc(Br)n2)cc1]

Copper(II) acetylacetonate (0.509 g, 1.945 mmol), cesium carbonate (12.7 g, 38.9 mmol), and (4-bromophenyl)(trifluoromethyl)sulfane (2.92 mL, 19.5 mmol) were combined in an oven-dried round bottom flask. Acetylacetone (0.799 mL, 7.78 mmol), DMF (40 mL) and 3-bromo-1H-1,2,4-triazole (3.45 g, 23.3 mmol) were added. The flask was evacuated under vacuum and backfilled with nitrogen thrice until bubbling subsided. The flask was placed in a pre-heated mantle at 90° C. and the reaction mixture was allowed to stir at that temperature for 18 h. LC-MS indicated incomplete conversion. The reaction mixture was allowed to cool to room temperature, and upon evacuation under vacuum, no bubbles formed, meaning the reaction remained well-deoxygenated. Additional copper(II) acetylacetonate (0.509 g) was added, and the reaction mixture was allowed to proceed at 100° C. for another 24 h. At this time, the reaction was stopped. After cooling to room temperature, the reaction mixture was diluted with water, causing a black solid to precipitate. The solid was collected by vacuum filtration and was washed with water. The collected solid was adsorbed onto diatomaceous earth. Purification by normal-phase high-performance liquid-chromatography, eluting with 0-5% methanol-DCM afforded the title compound as a white solid (2.4 g, 38%): ¹H NMR (300 MHz, CDCl₃) δ 8.50 (s, 1H), 7.82 (d, J=8.7 Hz, 2H), 7.78-7.70 (m, 2H); ¹⁹F NMR (376 MHz, CDCl₃) δ −42.47; ESIMS m/z 323 ([M−H]⁻).

The following compound was prepared in accordance to the procedure in Example 84:

### 4-(3-Bromo-1H-1,2,4-triazol-1-yl)-2-fluoroaniline (C447)

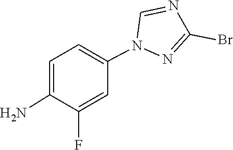 [SMILES: Nc1ccc(-n2cnc(Br)n2)cc1F]

The title compound was prepared and was isolated as a tan solid (2.6 g, 47%): ¹H NMR (500 MHz, CDCl₃) δ 8.27 (s, 1H), 7.32 (dd, J=11.2, 2.4 Hz, 1H), 7.19 (ddd, J=8.6, 2.4, 1.2 Hz, 1H), 6.85 (t, J=8.9 Hz, 1H), 3.93 (s, 2H); ¹⁹F NMR (471 MHz, CDCl₃) δ −132.13; ESIMS m/z 257 ([M+H]⁺).

### Example 85: Preparation of 3-bromo-1-phenyl-1H-1,2,4-triazole (C448)

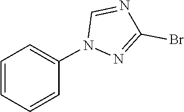 [SMILES: Brc1ncn(-c2ccccc2)n1]

To a 250 mL round bottom flask were added DCM (101 mL), phenylboronic acid (4.9 g, 40.6 mmol), 3-bromo-1H-1,2,4-triazole (3.0 g, 20.3 mmol), copper(II) acetate (5.5 g, 30.4 mmol), and pyridine (3.3 mL, 40.6 mmol). The reaction mixture was stirred at room temperature overnight. The reaction mixture was filtered over a pad of diatomaceous earth and concentrated under reduced pressure. Purification of the resulting material by column chromatography (silica gel) eluting with an EtOAc-Hexanes gradient afforded the title compound as a white solid (2.8 g, 61%): ¹H NMR (400 MHz, CDCl₃) δ 8.43 (s, 1H), 7.68-7.61 (m, 2H), 7.56-7.48 (m, 2H), 7.47-7.40 (m, 1H); ESIMS m/z 225 ([M+H]⁺).

The following compound was prepared in accordance to the procedure in Example 85:

### 3-Bromo-1-(4-fluorophenyl)-1H-1,2,4-triazole (C449)

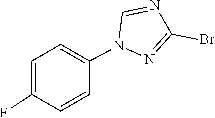 [SMILES: Fc1ccc(-n2cnc(Br)n2)cc1]

The title compound was prepared and was isolated as a white solid (3.5 g, 71%): ¹H NMR (400 MHz, CDCl₃) δ 8.37 (s, 1H), 7.67-7.58 (m, 2H), 7.25-7.16 (m, 2H); ¹⁹F NMR (376 MHz, CDCl₃) δ −111.74; ESIMS m/z 243 ([M+H]⁺).

### Example 86: Preparation of 4-bromo-1-(4-(trifluoromethoxy)phenyl)-1H-imidazole (C450)

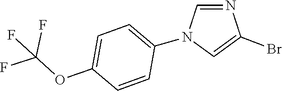 [SMILES: FC(F)(F)Oc1ccc(-n2cnc(Br)c2)cc1]

A round bottom flask was charged with 4-bromo-1H-imidazole (3.83 g, 26.0 mmol), copper(I) iodide (0.331 g, 1.736 mmol), quinolin-8-ol (0.252 g, 1.736 mmol), cesium carbonate (11.31 g, 34.7 mmol), and 1-iodo-4-(trifluoromethoxy)benzene (2.72 mL, 17.36 mmol). A 10:1 mixture of DMF (50 mL) and water (5.0 mL) were added to the reaction mixture, and the solution was heated to 130° C. overnight. After stirring 17 h, LC-MS indicated reaction completion. The reaction mixture was cooled to room temperature, was diluted with EtOAc, and was transferred to a separatory funnel with the aid of water. The layers were separated, and the aqueous layer was extracted with EtOAc. The combined organic layers were washed with saturated aqueous sodium bicarbonate and then ammonium chloride, were dried over Na₂SO₄/magnesium sulfate (MgSO₄) and were concentrated in vacuo. Purification of the residue by normal-phase high-performance liquid chromatography, eluting with 0-10% methanol-DCM afforded the title compound as an off-white solid (3.01 g, 56%): 1H NMR (400 MHz, CDCl₃) δ 7.70 (d, J=1.6 Hz, 1H), 7.41 (d, J=9.0 Hz, 2H), 7.36 (d, J=8.9 Hz, 2H), 7.24 (d, J=1.5 Hz, 1H); ¹⁹F NMR (376 MHz, CDCl₃) δ −58.05; ESIMS m/z 307 ([M+H]⁺).

The following compounds were prepared in accordance to the procedure in Example 86:

### 2-(4-Bromo-1H-imidazol-1-yl)-5-(trifluoromethyl)pyridine (C451)

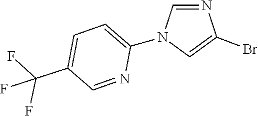 [SMILES: FC(F)(F)c1ccc(-n2cnc(Br)c2)nc1]

The title compound was prepared and was isolated as an off-white solid (1.58 g, 24%): ¹H NMR (400 MHz, CDCl₃) δ 8.76 (t, J=1.4 Hz, 1H), 8.31 (d, J=1.5 Hz, 1H), 8.13-8.05 (m, 1H), 7.67 (d, J=1.5 Hz, 1H), 7.44 (d, J=8.6 Hz, 1H); ¹⁹F NMR (376 MHz, CDCl₃) δ −62.17; ESIMS m/z 292 ([M+H]⁺).

### 4-Bromo-1-(4-(trifluoromethyl)phenyl)-1H-imidazole (C452)

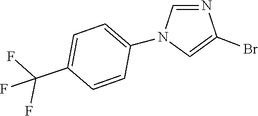 [SMILES: FC(F)(F)c1ccc(-n2cnc(Br)c2)cc1]

The title compound was prepared and was isolated as an orange solid (2.34 g, 43%): ¹H NMR (400 MHz, CDCl₃) δ 7.80-7.73 (m, 3H), 7.57-7.47 (m, 2H), 7.31 (t, J=1.3 Hz, 1H); ¹⁹F NMR (376 MHz, CDCl₃) δ −62.60; ESIMS m/z 291 ([M+H]⁺).

### 3-Bromo-1-(4-(difluoromethoxy)phenyl)-1H-1,2,4-triazole (C453)

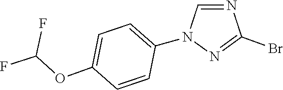 [SMILES: FC(F)Oc1ccc(-n2cnc(Br)n2)cc1]

The title compound was prepared and was isolated as a white solid (2 g, 37%): ¹H NMR (400 MHz, CDCl₃) δ 8.39 (s, 1H), 7.71-7.60 (m, 2H), 7.35-7.27 (m, 2H), 6.56 (t, J=73.0 Hz, 1H); ¹⁹F NMR (376 MHz, CDCl₃) δ −81.53; ESIMS m/z 291 ([M+H]⁺).

### 3-Bromo-1-(4-(difluoromethyl)phenyl)-1H-1,2,4-triazole (C454)

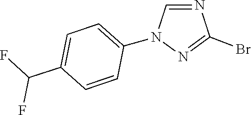 [SMILES: FC(F)c1ccc(-n2cnc(Br)n2)cc1]

The title compound was prepared and was isolated as a white solid (1.43 g, 40%): ¹H NMR (400 MHz, CDCl₃) δ 8.49 (t, J=1.4 Hz, 1H), 7.84-7.62 (m, 4H), 6.90-6.50 (m, 1H); ¹⁹F NMR (376 MHz, CDCl₃) δ −111.39; ESIMS m/z 275 ([M+H]⁺).

### 2-(3-Bromo-1H-1,2,4-triazol-1-yl)-5-(trifluoromethyl)pyridine (C455)

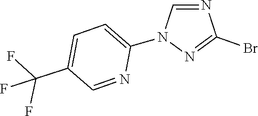 [SMILES: FC(F)(F)c1ccc(-n2cnc(Br)n2)nc1]

The title compound was prepared and was isolated as a white solid (2.8 g, 41%): ¹H NMR (400 MHz, CDCl₃) δ 9.11 (s, 1H), 8.74 (dp, J=2.5, 0.8 Hz, 1H), 8.18-8.12 (m, 1H), 8.01 (dt, J=8.5, 0.8 Hz, 1H); ¹⁹F NMR (376 MHz, CDCl₃) δ −62.21; ESIMS m/z 294 ([M+H]⁺).

### 2-(3-Bromo-1H-1,2,4-triazol-1-yl)-5-(difluoromethyl)pyridine (C456)

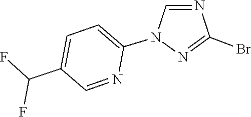 [SMILES: FC(F)c1ccc(-n2cnc(Br)n2)nc1]

The title compound was prepared and was isolated as a white solid (2.8 g, 42%): ¹H NMR (400 MHz, CDCl₃) δ 9.09 (s, 1H), 8.60 (p, J=1.1 Hz, 1H), 8.06 (ddd, J=8.5, 2.3, 1.2 Hz, 1H), 7.98 (dd, J=8.4, 0.9 Hz, 1H), 6.78 (t, J=55.6 Hz, 1H); ¹⁹F NMR (376 MHz, CDCl₃) δ −112.35; ESIMS m/z 276 ([M+H]⁺).

### Example 87: Preparation of 3-(3-bromo-1H-pyrazol-1-yl)-6-(trifluoromethyl)pyridazine (C457)

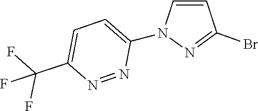 [SMILES: FC(F)(F)c1ccc(-n2ccc(Br)n2)nn1]

3-Chloro-6-(trifluoromethyl)pyridazine (1.9 g, 5.06 mmol), 3-bromo-1H-pyrazole (2.25 g, 10.4 mmol) and cesium carbonate (7.0 g, 21.4 mmol) were placed in a round-bottom flask, and N,N-dimethylacetamide (DMA; 25 mL) was added. The mixture was stirred and heated at 100° C. for 6 h. The reaction mixture was cooled to room temperature and diluted with EtOAc (100 mL). The organic layer was washed with water and brine, dried over Na₂SO₄ and concentrated under reduced pressure. Purification of the residue flash silica gel chromatography eluting with 10-70% EtOAc in hexane yielded the title compound (1.0 g, 31%): ¹H NMR (CDCl₃) δ: 8.74 (d, J=2.7 Hz, 1H), 8.33 (d, J=9.1 Hz, 1H), 7.95 (d, J=9.2 Hz, 1H), 6.62 (d, J=2.7 Hz, 1H); LC-MS m/z 308 (M+1).

### Example 88: Preparation of 3-(3-bromo-1H-1,2,4-triazol-1-yl)-6-(trifluoromethyl)pyridazine (C458)

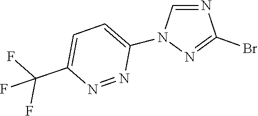 [SMILES: FC(F)(F)c1ccc(-n2cnc(Br)n2)nn1]

To 60% NaH (1.5 g, 37 mmol) in DMF (20 mL) at 0° C. was added 3-bromo-1H-triazole (6 g, 41 mmol). The reaction mixture was stirred at 0° C. for 30 minutes, at which point 3-chloro-6-(trifluoromethyl)pyridazine (2.5 g, 13.7 mmol) was added. The reaction mixture was heated at 130° C. for 1 h. The reaction mixture was cooled to room temperature and quenched with water at 0° C. The product precipitated and was collected using a sintered funnel and washed with hexanes. The title compound was isolated as a white solid (2 g, 50%): ¹H NMR (CDCl₃) δ 9.34 (s, 1H), 8.28 (d, J=9.1 Hz, 1H), 8.06 (d, J=9.1 Hz, 1H).

### Example 89: Preparation of 3-bromo-1-(4-((trifluoromethyl)sulfonyl)phenyl)-1H-1,2,4-triazole (C459)

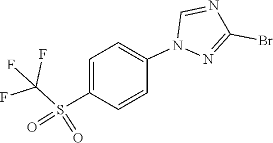 [SMILES: O=S(=O)(c1ccc(-n2cnc(Br)n2)cc1)C(F)(F)F]

3-Bromo-1-(4-((trifluoromethyl)thio)phenyl)-1H-1,2,4-triazole (C446; 3.00 g, 9.26 mmol) was dissolved in acetic acid (46.3 mL), and hydrogen peroxide (50% solution in water; 19.9 mL, 324 mmol) was added slowly via syringe at room temperature. The flask was placed on a pre-heated heating block and heated at reflux (130° C.) for 3 h. The reaction mixture was allowed to cool to room temperature and was diluted with DCM (50 mL), causing the mixture to become cloudy. The mixture was transferred to a separatory funnel with the aid of water, and the layers were separated. The aqueous layer was extracted with DCM (2×50 mL). The combined organic layers were washed with saturated aqueous sodium bisulfite and saturated aqueous sodium bicarbonate, were passed through a phase separator, and concentrated in vacuo to afford a pale yellow oil that slowly began to crystallize into white solids. This precipitating oil was taken up in a minimal amount of EtOAc, and heptanes were added, forming a white cloudy solution, from which white solids began to precipitate. This process was aided by the further addition of hexanes. The white crystals were collected by vacuum filtration, washed with hexanes, and dried under high vacuum overnight. The title compound was isolated as a white solid (2.916 g, 84%), which required no further purification: ¹H NMR (400 MHz, CDCl₃) δ 8.63 (s, 1H), 8.21 (d, J=8.8 Hz, 2H), 8.02 (d, J=8.9 Hz, 1H); ¹⁹F NMR (376 MHz, CDCl₃) δ −78.04; ESIMS m/z 356 ([M+H]⁺).

The following compound was prepared in accordance to the procedure in Example 89:

### 1-Fluoro-4-((trifluoromethyl)sulfonyl)benzene (C460)

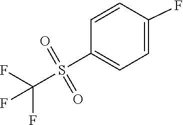 [SMILES: O=S(=O)(c1ccc(F)cc1)C(F)(F)F]

The title compound was prepared and was isolated as a pale yellow oil (620 mg, 11%): ¹H NMR (400 MHz, CDCl₃) δ 8.12-8.04 (m, 2H), 7.42-7.31 (m, 2H); ¹⁹F NMR (376 MHz, CDCl₃) δ −78.40, −97.60; ESIMS m/z 229 ([M+H]⁺).

### Example 90: Preparation of 3-bromo-1-(4-((trifluoromethyl)sulfinyl)phenyl)-1H-1,2,4-triazole (C461)

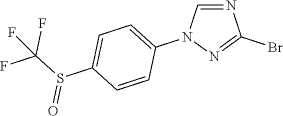 [SMILES: O=S(c1ccc(-n2cnc(Br)n2)cc1)C(F)(F)F]

3-Bromo-1-(4-((trifluoromethyl)thio)phenyl)-1H-1,2,4-triazole (C446; 1.50 g, 4.63 mmol) was dissolved in DCM (23 mL), and the reaction mixture was cooled in an ice bath. m-Chloroperoxybenzoic acid (mCPBA; 1.141 g, 5.09 mmol) was added portionwise (6×190 mg portions added every 90 seconds), taking care to maintain the internal temperature below 3° C. The reaction mixture was allowed to warm slowly to room temperature overnight. After stirring 16 h, the reaction mixture was diluted with DCM, quenched with water, and transferred to a separatory funnel. The layers were separated, and the aqueous layer was extracted with DCM, forming an emulsion. Addition of brine helped break the emulsion. The aqueous layer was extracted once with DCM. The combined organic layers were passed through a phase separator and concentrated in vacuo to afford a very sticky solid. This solid was purified by normal-phase high-performance liquid chromatography, eluting with 0-5% methanol-DCM. The title compound was isolated along with the m-chlorobenzoic acid byproduct. This impure mixture was taken up in Et₂O and transferred to a separatory funnel, where it was washed 1 N NaOH. The organic layer was passed through a phase separator and concentrated. The residue was dissolved in a minimal amount of EtOAc and triturated with heptanes to provide the title compound as a white solid (806 mg, 51%): ¹H NMR (400 MHz, CDCl₃) δ 8.58 (s, 1H), 7.96 (d, J=1.6 Hz, 4H); ¹⁹F NMR (376 MHz, CDCl₃) δ −74.06; ESIMS m/z 340 ([M+H]⁺).

### Example 91: Preparation of 4-(1-(4-((trifluoromethyl)thio)phenyl)-1H-1,2,4-triazol-3-yl)aniline (C462)

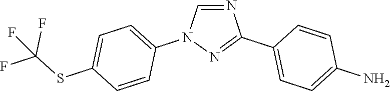 [SMILES: Nc1ccc(-c2ncn(-c3ccc(SC(F)(F)F)cc3)n2)cc1]

Tetrakis(triphenylphosphine)palladium(0) (0.357 g, 0.309 mmol), sodium bicarbonate (0.778 g, 9.26 mmol), 4-(4,4,5,5-tetramethyl-1,3,2-dioxaborolan-2-yl)aniline (0.811 g, 3.70 mmol) and 3-bromo-1-(4-((trifluoromethyl)thio)phenyl)-1H-1,2,4-triazole (C446; 1.00 g, 3.09 mmol) were combined in an oven-dried round bottom flask and dissolved in dioxane (12.3 mL) and water (3.1 mL). The reaction mixture was allowed to stir at 100° C. overnight. After stirring overnight, LC-MS indicated reaction completion. The reaction mixture was allowed to cool to room temperature, diluted with EtOAc, and transferred to a separatory funnel with the aid of water. The layers were separated, and the aqueous layer was extracted twice with EtOAc. The combined organic layers were washed with brine, passed through a phase separator, and concentrated in vacuo to afford a brown oil. Purification of the residue by normal-phase high-performance liquid chromatography eluting with 0-5% methanol-DCM provided a residue that was dissolved in the minimal amount of EtOAc. Heptanes were added, causing a solid to precipitate. The solid was collected by vacuum filtration, washed with hexanes, and dried under high vacuum. The title compound was isolated as a tan solid (397 mg, 36%): ¹H NMR (400 MHz, CDCl₃) δ 8.59 (s, 1H), 8.00 (d, J=8.5 Hz, 2H), 7.88-7.74 (m, 4H), 6.82-6.72 (m, 2H), 3.87 (s, 2H); ¹⁹F NMR (376 MHz, CDCl₃) δ−42.73; ESIMS m/z 337 ([M+H]⁺).

The following compounds were prepared in accordance to the procedure in Example 91:

### 2-Fluoro-4-(1-(4-((trifluoromethyl)sulfonyl)phenyl)-1H-1,2,4-triazol-3-yl)aniline (C463)

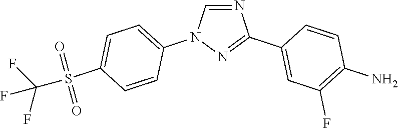 [SMILES: Nc1ccc(-c2ncn(-c3ccc(S(=O)(=O)C(F)(F)F)cc3)n2)cc1F]

The title compound was prepared and was isolated as an off-white solid (886 mg, 78%): mp 187-189° C.; ¹H NMR (400 MHz, CDCl₃) δ 8.70 (s, 1H), 8.20 (d, J=8.6 Hz, 2H), 8.09 (d, J=8.9 Hz, 2H), 7.88-7.78 (m, 2H), 6.86 (t, J=8.5 Hz, 1H), 3.98 (s, 2H); ¹⁹F NMR (376 MHz, CDCl₃) δ −78.18, −135.19; ESIMS m/z 387 ([M+H]⁺).

### 2-Methyl-4-(1-(4-((trifluoromethyl)sulfonyl)phenyl)-1H-1,2,4-triazol-3-yl)aniline (C464)

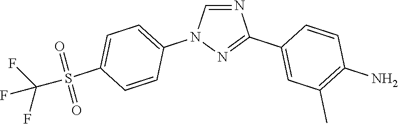 [SMILES: Cc1cc(-c2ncn(-c3ccc(S(=O)(=O)C(F)(F)F)cc3)n2)ccc1N]

The title compound was prepared and was isolated as a tan solid (645 mg, 59%): ¹H NMR (400 MHz, CDCl₃) δ 8.70 (s, 1H), 8.19 (d, J=8.7 Hz, 2H), 8.12-8.06 (m, 2H), 7.95-7.85 (m, 2H), 6.76 (d, J=8.2 Hz, 1H), 3.87 (s, 2H), 2.26 (s, 3H); ¹⁹F NMR (376 MHz, CDCl₃) δ −78.22; ESIMS m/z 383 ([M+H]⁺).

### 2-Amino-5-(1-(4-((trifluoromethyl)sulfonyl)phenyl)-1H-1,2,4-triazol-3-yl)benzonitrile (C465)

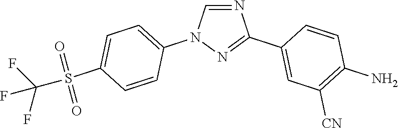 [SMILES: N#Cc1cc(-c2ncn(-c3ccc(S(=O)(=O)C(F)(F)F)cc3)n2)ccc1N]

The title compound was prepared and was isolated as a white solid (531 mg, 54%): ¹H NMR (400 MHz, CDCl₃) δ 8.72 (s, 1H), 8.28 (d, J=1.9 Hz, 1H), 8.21 (d, J=8.8 Hz, 2H), 8.17 (dd, J=8.7, 2.0 Hz, 1H), 8.09 (d, J=8.8 Hz, 2H), 6.86 (d, J=8.7 Hz, 1H), 4.66 (s, 2H); ¹⁹F NMR (376 MHz, CDCl₃) δ −78.14; ESIMS m/z 394 ([M+H]⁺).

### 2-Fluoro-4-(1-(4-((trifluoromethyl)sulfinyl)phenyl)-1H-1,2,4-triazol-3-yl)aniline (C466)

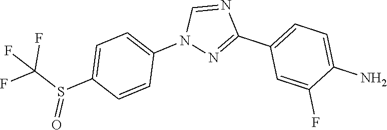 [SMILES: Nc1ccc(-c2ncn(-c3ccc(S(=O)C(F)(F)F)cc3)n2)cc1F]

The title compound was prepared and was isolated as an off-white solid (498 mg, 52%): ¹H NMR (500 MHz, CDCl₃) δ 8.65 (s, 1H), 8.03 (d, J=8.8 Hz, 2H), 7.96 (d, J=8.5 Hz, 2H), 7.87-7.78 (m, 2H), 6.86 (t, J=8.5 Hz, 1H), 3.95 (s, 2H); ¹⁹F NMR (376 MHz, CDCl₃) δ −74.26, −135.27; ESIMS m/z 371 ([M+H]⁺).

### 2-Fluoro-4-(1-(4-(pentafluoro-λ⁶-sulfaneyl)phenyl)-1H-imidazol-4-yl)aniline (C467)

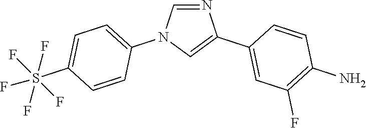 [SMILES: Nc1ccc(-c2cn(-c3ccc(S(F)(F)(F)(F)F)cc3)cn2)cc1F]

The title compound was prepared and was isolated as a pink solid (909 mg, 55%): ¹H NMR (400 MHz, CDCl₃) δ 7.93-7.90 (m, 2H), 7.90 (d, J=2.1 Hz, 1H), 7.53 (d, J=8.7 Hz, 2H), 7.49 (dd, J=12.2, 1.9 Hz, 1H), 7.45 (d, J=1.4 Hz, 1H), 7.44-7.39 (m, 1H), 6.82 (dd, J=9.2, 8.2 Hz, 1H), 3.78 (s, 2H); ¹⁹F NMR (376 MHz, CDCl₃) δ −135.13; ESIMS m/z 380 ([M+H]⁺).

### 2-Methyl-4-(1-(4-(pentafluoro-λ⁶-sulfaneyl)phenyl)-1H-imidazol-4-yl)aniline (C468)

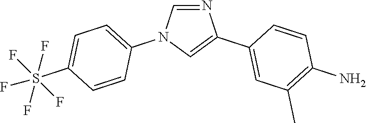 [SMILES: Cc1cc(-c2cn(-c3ccc(S(F)(F)(F)(F)F)cc3)cn2)ccc1N]

The title compound was prepared and was isolated as a grey solid (364 mg, 22%): mp 138-142° C.; ¹H NMR (400 MHz, CDCl₃) δ 7.92 (d, J=1.4 Hz, 1H), 7.90 (d, J=9.0 Hz, 2H), 7.58 (s, 1H), 7.53 (d, J=8.8 Hz, 2H), 7.53-7.46 (m, 1H), 7.45 (d, J=1.4 Hz, 1H), 6.73 (d, J=8.2 Hz, 1H), 3.68 (s, 2H), 2.23 (s, 3H); ESIMS m/z 376 ([M+H]⁺).

### 4-(3-(4-(Trifluoromethoxy)phenyl)-1H-1,2,4-triazol-1-yl)aniline (C469)

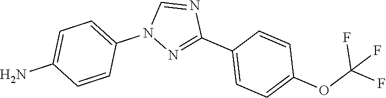 [SMILES: Nc1ccc(-n2cnc(-c3ccc(OC(F)(F)F)cc3)n2)cc1]

The title compound was prepared and was isolated as a yellow solid (522 mg, 37%): ¹H NMR (400 MHz, CDCl₃) δ 8.42 (s, 1H), 8.21 (d, J=8.8 Hz, 2H), 7.48 (d, J=8.7 Hz, 2H), 6.78 (d, J=8.7 Hz, 2H), 3.86 (s, 2H); ¹⁹F NMR (376 MHz, CDCl₃) δ −57.70; ESIMS m/z 321 ([M+H]⁺).

### 1-(3-Methyl-4-nitrophenyl)-3-(4-(trifluoromethoxy)phenyl)-1H-1,2,4-triazole (C470)

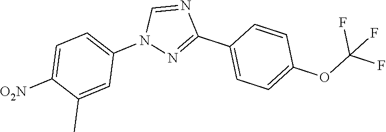 [SMILES: Cc1cc(-n2cnc(-c3ccc(OC(F)(F)F)cc3)n2)ccc1[N+](=O)[O-]]

The title compound was prepared and was isolated as a pale yellow solid (887 mg, 66%): ¹H NMR (400 MHz, CDCl₃) δ 8.68 (s, 1H), 8.27-8.23 (m, 2H), 8.21 (d, J=8.8 Hz, 1H), 7.84-7.79 (m, 1H), 7.75 (dd, J=8.9, 2.4 Hz, 1H), 7.38-7.31 (m, 2H), 2.75 (s, 3H); ¹⁹F NMR (376 MHz, CDCl₃) δ −57.70; ESIMS m/z 365 ([M+H]⁺).

### 2-Fluoro-4-(1-(4-(trifluoromethoxy)phenyl)-1H-imidazol-4-yl)aniline (C471)

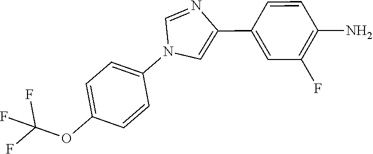 [SMILES: Nc1ccc(-c2cn(-c3ccc(OC(F)(F)F)cc3)cn2)cc1F]

The title compound was prepared and was isolated as a crimson solid (208 mg, 19%): ¹H NMR (400 MHz, CDCl₃) δ 7.83 (d, J=1.4 Hz, 1H), 7.51-7.44 (m, 3H), 7.43-7.38 (m, 2H), 7.38-7.33 (m, 2H), 6.82 (dd, J=9.2, 8.2 Hz, 1H), 3.77 (s, 2H); ¹⁹F NMR (376 MHz, CDCl₃) δ −58.04, −135.17; ESIMS m/z 338 ([M+H]⁺).

### 2-Methyl-4-(1-(4-(trifluoromethoxy)phenyl)-1H-imidazol-4-yl)aniline (C472)

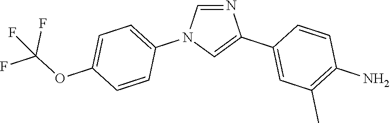 [SMILES: Cc1cc(-c2cn(-c3ccc(OC(F)(F)F)cc3)cn2)ccc1N]

The title compound was prepared and was isolated as a pink solid (922 mg, 85%): ¹H NMR (400 MHz, CDCl₃) δ 7.83 (d, J=1.5 Hz, 1H), 7.75-7.62 (m, 2H), 7.57 (s, 1H), 7.48-7.46 (m, 2H), 7.41-7.33 (m, 2H), 6.72 (d, J=8.2 Hz, 1H), 3.66 (s, 2H), 2.23 (s, 3H); ¹⁹F NMR (376 MHz, CDCl₃) δ −58.06; ESIMS m/z 334 ([M+H]⁺).

### 2-Fluoro-4-(1-(4-(trifluoromethyl)phenyl)-1H-imidazol-4-yl)aniline (C473)

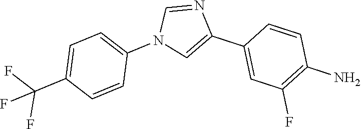 [SMILES: Nc1ccc(-c2cn(-c3ccc(C(F)(F)F)cc3)cn2)cc1F]

The title compound was prepared and was isolated as a pink solid (92 mg, 12%): ¹H NMR (400 MHz, CDCl₃) δ 7.92 (d, J=1.5 Hz, 1H), 7.78 (d, J=8.4 Hz, 2H), 7.62-7.53 (m, 2H), 7.53-7.44 (m, 2H), 7.42 (dt, J=8.3, 1.2 Hz, 1H), 6.82 (dd, J=9.1, 8.2 Hz, 1H), 3.78 (s, 2H); ¹⁹F NMR (376 MHz, CDCl₃) δ −62.45, −135.14; ESIMS m/z 322 ([M+H]⁺).

### 2-Fluoro-4-(1-(5-(trifluoromethyl)pyridin-2-yl)-1H-imidazol-4-yl)aniline (C474)

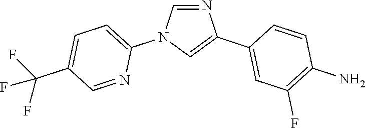 [SMILES: Nc1ccc(-c2cn(-c3ccc(C(F)(F)F)cn3)cn2)cc1F]

The title compound was prepared and was isolated as a purple solid (97 mg, 16%): ¹H NMR (400 MHz, CDCl₃) δ 8.76 (dt, J=2.1, 0.9 Hz, 1H), 8.42 (d, J=1.4 Hz, 1H), 8.07 (dd, J=8.6, 2.4 Hz, 1H), 7.82 (d, J=1.4 Hz, 1H), 7.51-7.42 (m, 3H), 6.83 (dd, J=9.2, 8.2 Hz, 1H), 3.79 (s, 2H); ¹⁹F NMR (376 MHz, CDCl₃) δ −62.07, −135.16; ESIMS m/z 323 ([M+H]⁺).

### 2-Methyl-4-(1-(5-(trifluoromethyl)pyridin-2-yl)-1H-imidazol-4-yl)aniline (C475)

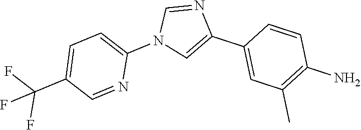 [SMILES: Cc1cc(-c2cn(-c3ccc(C(F)(F)F)cn3)cn2)ccc1N]

The title compound was prepared and was isolated as a tan solid (470 mg, 61%): ¹H NMR (400 MHz, CDCl₃) δ 8.75 (dt, J=2.5, 0.9 Hz, 1H), 8.42 (d, J=1.4 Hz, 1H), 8.08-8.02 (m, 1H), 7.80 (d, J=1.4 Hz, 1H), 7.60 (dd, J=2.0, 0.9 Hz, 1H), 7.52 (dd, J=8.1, 2.1 Hz, 1H), 7.48 (d, J=8.4 Hz, 1H), 6.73 (d, J=8.1 Hz, 1H), 3.69 (s, 2H), 2.23 (s, 3H); ¹⁹F NMR (376 MHz, CDCl₃) δ −62.04; ESIMS m/z 319 ([M+H]⁺).

### 2-Methyl-4-(1-(4-(trifluoromethyl)phenyl)-1H-imidazol-4-yl)aniline (C476)

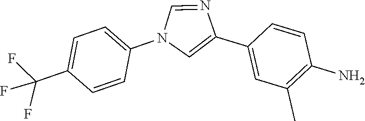 [SMILES: Cc1cc(-c2cn(-c3ccc(C(F)(F)F)cc3)cn2)ccc1N]

The title compound was prepared and was isolated as an off-yellow solid (300 mg, 27%): ¹H NMR (400 MHz, CDCl₃) δ 7.92 (d, J=1.4 Hz, 1H), 7.76 (d, J=8.6 Hz, 2H), 7.67 (ddd, J=12.0, 8.3, 1.4 Hz, 1H), 7.59-7.54 (m, 3H), 7.46 (d, J=1.5 Hz, 1H), 6.73 (d, J=8.2 Hz, 1H), 3.68 (s, 2H), 2.23 (s, 3H); ¹⁹F NMR (376 MHz, CDCl₃) δ −62.41; ESIMS m/z 318 ([M+H]⁺).

### 4-(1-(4-(Trifluoromethyl)phenyl)-1H-imidazol-4-yl)aniline (C477)

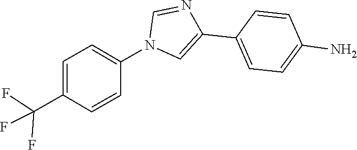 [SMILES: Nc1ccc(-c2cn(-c3ccc(C(F)(F)F)cc3)cn2)cc1]

The title compound was prepared and was isolated as an off-orange solid (164 mg, 24%): ¹H NMR (400 MHz, CDCl₃) δ 7.92 (d, J=1.5 Hz, 1H), 7.77 (d, J=8.5 Hz, 2H), 7.67-7.62 (m, 2H), 7.56 (d, J=8.3 Hz, 2H), 7.46 (d, J=1.4 Hz, 1H), 6.77-6.71 (m, 2H), 3.72 (d, J=10.6 Hz, 2H); ¹⁹F NMR (376 MHz, CDCl₃) δ −62.42; ESIMS m/z 304 ([M+H]⁺).

### 4-(1-(5-(Trifluoromethyl)pyridin-2-yl)-1H-imidazol-4-yl)aniline (C478)

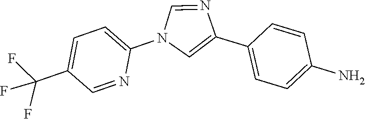 [SMILES: Nc1ccc(-c2cn(-c3ccc(C(F)(F)F)cn3)cn2)cc1]

The title compound was prepared and was isolated as a pink solid (656 mg, 62%): ¹H NMR (400 MHz, CDCl₃) δ 8.78-8.73 (m, 1H), 8.42 (d, J=1.3 Hz, 1H), 8.06 (dd, J=8.6, 2.4 Hz, 1H), 7.80 (d, J=1.4 Hz, 1H), 7.71-7.63 (m, 2H), 7.48 (d, J=8.6 Hz, 1H), 6.79-6.71 (m, 2H), 3.74 (s, 2H); ¹⁹F NMR (376 MHz, CDCl₃) δ −62.05; ESIMS m/z 305 ([M+H]⁺).

### Example 92: Preparation of 2-fluoro-4-(1-(4-((trifluoromethyl)sulfonyl)phenyl)-1H-imidazol-4-yl)aniline (C479)

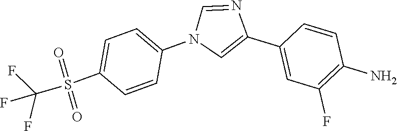 [SMILES: Nc1ccc(-c2cn(-c3ccc(S(=O)(=O)C(F)(F)F)cc3)cn2)cc1F]

4-Bromo-1-(4-((trifluoromethyl)sulfonyl)phenyl)-1H-imidazole (C484; 600 mg, 1.69 mmol), 2-fluoro-4-(4,4,5,5-tetramethyl-1,3,2-dioxaborolan-2-yl)aniline (501 mg, 2.11 mmol), Xphos-Pd-G3 precatalyst (71 mg, 0.084 mmol) and potassium phosphate tribasic (1076 mg, 5.07 mmol) were combined in a 40 mL vial and dissolved in dioxane (7.68 mL) and water (768 μL). The vial was evacuated and backfilled several times with nitrogen. The reaction mixture was heated to 90° C. and was allowed to stir overnight. After 17 h, LC-MS indicated complete conversion. The reaction mixture was allowed to cool to room temperature, diluted with EtOAc, passed through a plug of diatomaceous earth, and washed with EtOAc and water. The biphasic filtrate was transferred to a separatory funnel with the aid of EtOAc and water. The layers were separated, and the aqueous layer was extracted with EtOAc. The combined organic layers were washed with brine, dried over Na₂SO₄— magnesium sulfate, and concentrated in vacuo to afford a dark oil. Purification of the residue by normal-phase high-performance liquid chromatography, eluting with 0-5% methanol-DCM afforded the title compound as a yellow solid (478 mg, 72%): ¹H NMR (400 MHz, CDCl₃) δ 8.18 (d, J=8.6 Hz, 2H), 8.02 (d, J=1.4 Hz, 1H), 7.73 (d, J=8.7 Hz, 2H), 7.55-7.46 (m, 2H), 7.43 (d, J=8.5 Hz, 1H), 6.83 (t, J=8.6 Hz, 1H), 3.81 (s, 2H); ¹⁹F NMR (376 MHz, CDCl₃) δ −78.16, −135.00; ESIMS m/z 386 ([M+H]⁺).

The following compounds were prepared in accordance to the procedure in Example 92:

### 2-Chloro-4-(3-(4-(trifluoromethoxy)phenyl)-1H-1,2,4-triazol-1-yl)aniline (C480)

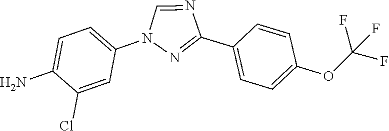 [SMILES: Nc1ccc(-n2cnc(-c3ccc(OC(F)(F)F)cc3)n2)cc1Cl]

The title compound was prepared and was isolated as a tan solid (665 mg, 56%): ¹H NMR (400 MHz, CDCl₃) δ 8.42 (s, 1H), 8.28-8.18 (m, 2H), 7.66 (d, J=2.5 Hz, 1H), 7.41 (dd, J=8.6, 2.5 Hz, 1H), 7.31 (d, J=8.3 Hz, 2H), 6.87 (d, J=8.6 Hz, 1H), 4.25 (s, 2H); ¹⁹F NMR (376 MHz, CDCl₃) δ −57.70; ESIMS m/z 355 ([M+H]⁺).

### 2-Fluoro-4-(3-(4-(trifluoromethoxy)phenyl)-1H-1,2,4-triazol-1-yl)aniline (C481)

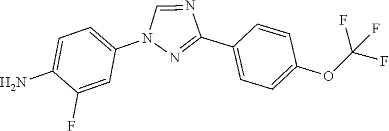 [SMILES: Nc1ccc(-n2cnc(-c3ccc(OC(F)(F)F)cc3)n2)cc1F]

The title compound was prepared and was isolated as an off-white solid (932 mg, 67%): ¹H NMR (400 MHz, CDCl₃) δ 8.43 (s, 1H), 8.25-8.17 (m, 2H), 7.43 (dd, J=11.3, 2.4 Hz, 1H), 7.30 (dddd, J=7.5, 6.5, 2.2, 1.0 Hz, 3H), 6.88 (t, J=8.9 Hz, 1H), 3.91 (s, 2H); ¹⁹F NMR (376 MHz, CDCl₃) δ −57.71, −132.27; ESIMS m/z 339 ([M+H]⁺).

### 2-Fluoro-4-(3-(4-(trifluoromethyl)phenyl)-1H-1,2,4-triazol-1-yl)aniline (C482)

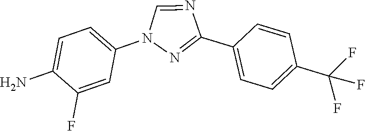 [SMILES: Nc1ccc(-n2cnc(-c3ccc(C(F)(F)F)cc3)n2)cc1F]

The title compound was prepared and was isolated as a beige solid (845 mg, 67%): 1H NMR (400 MHz, CDCl₃) δ 8.46 (s, 1H), 8.33-8.26 (m, 2H), 7.72 (d, J=8.2 Hz, 2H), 7.44 (dd, J=11.3, 2.4 Hz, 1H), 7.30 (ddd, J=8.6, 2.5, 1.2 Hz, 1H), 6.89 (t, J=8.9 Hz, 1H), 3.95-3.90 (m, 2H); ¹⁹F NMR (376 MHz, CDCl₃) δ −62.66, −132.22; ESIMS m/z 323 ([M+H]⁺).

### 2-Chloro-4-(4-(4-(trifluoromethoxy)phenyl)-1H-imidazol-1-yl)aniline (C483)

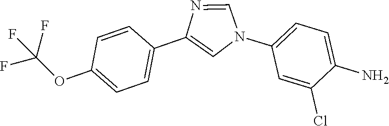 [SMILES: Nc1ccc(-n2cnc(-c3ccc(OC(F)(F)F)cc3)c2)cc1Cl]

The title compound was prepared and was isolated as a tan solid (549 mg, 42%): ¹H NMR (400 MHz, CDCl₃) δ 7.90-7.78 (m, 2H), 7.75 (d, J=1.4 Hz, 1H), 7.43 (d, J=1.4 Hz, 1H), 7.36 (d, J=2.4 Hz, 1H), 7.27-7.22 (m, 2H), 7.15 (dd, J=8.6, 2.5 Hz, 1H), 6.85 (d, J=8.5 Hz, 1H), 4.22 (s, 2H); ¹⁹F NMR (376 MHz, CDCl₃) δ −57.81; ESIMS m/z 354 ([M+H]⁺).

### Example 93: Preparation of 4-bromo-1-(4-((trifluoromethyl)sulfonyl)phenyl)-1H-imidazole (C484)

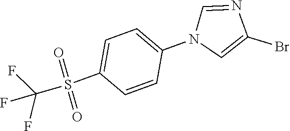 [SMILES: O=S(=O)(c1ccc(-n2cnc(Br)c2)cc1)C(F)(F)F]

1-Fluoro-4-((trifluoromethyl)sulfonyl)benzene (C460; 625 mg, 2.74 mmol) was dissolved in DMF (6.09 mL), and 4-bromo-1H-imidazole (483 mg, 3.29 mmol) was added, followed by potassium carbonate (568 mg, 4.11 mmol). The reaction was allowed to proceed at 100° C. overnight. After stirring overnight, LC-MS indicated reaction completion. The reaction mixture was allowed to cool to room temperature and water was added, causing a solid to precipitate. The solid was collected by vacuum filtration and was washed with water and then hexanes. The collected solid was dried under high vacuum. The title product was isolated as a white solid (610 mg, 62%): ¹H NMR (400 MHz, CDCl₃) δ 8.20 (d, J=8.5 Hz, 2H), 7.89 (d, J=1.6 Hz, 1H), 7.71-7.60 (m, 2H), 7.39 (d, J=1.6 Hz, 1H); ¹⁹F NMR (376 MHz, CDCl₃) δ −78.04; ESIMS m/z 355 ([M+H]⁺).

The following compounds were prepared in accordance to the procedure in Example 93:

### 4-Bromo-1-(4-(pentafluoro-λ⁶-sulfaneyl)phenyl)-1H-imidazole (C485)

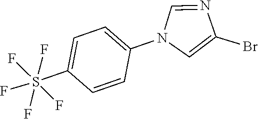 [SMILES: FS(F)(F)(F)(F)c1ccc(-n2cnc(Br)c2)cc1]

The title compound was prepared and was isolated as a white solid (3.68 g, 77%): mp 154-156° C.; ¹H NMR (400 MHz, CDCl₃) δ 7.96-7.85 (m, 2H), 7.78 (d, J=1.6 Hz, 1H), 7.48 (d, J=8.7 Hz, 2H), 7.31 (d, J=1.6 Hz, 1H); ESIMS m/z 349 ([M+H]⁺).

### 3-Bromo-1-(4-nitrophenyl)-1H-1,2,4-triazole (C486)

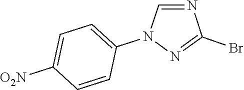 [SMILES: O=[N+]([O-])c1ccc(-n2cnc(Br)n2)cc1]

The title compound was prepared and was isolated as a pale yellow solid (4.98 g, 86%): mp 190-192° C.; ¹H NMR (400 MHz, CDCl₃) δ 8.57 (s, 1H), 8.47-8.38 (m, 2H), 7.92-7.85 (m, 2H); ESIMS m/z 268 ([M−H]⁻).

### 3-Bromo-1-(3-methyl-4-nitrophenyl)-1H-1,2,4-triazole (C487)

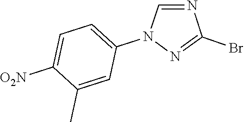 [SMILES: Cc1cc(-n2cnc(Br)n2)ccc1[N+](=O)[O-]]

The title compound was prepared and was isolated as an orange solid (5.73 g, 95%): mp 193-195° C.; ¹H NMR (400 MHz, CDCl₃) δ 8.54 (s, 1H), 8.17 (d, J=8.8 Hz, 1H), 7.72 (d, J=2.5 Hz, 1H), 7.64 (dd, J=8.8, 2.5 Hz, 1H), 2.71 (s, 3H); ESIMS m/z 283 ([M+H]⁺).

### 3-Bromo-1-(3-chloro-4-nitrophenyl)-1H-1,2,4-triazole (C₄₈₈)

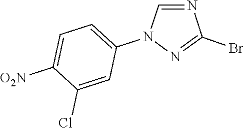 [SMILES: O=[N+]([O-])c1ccc(-n2cnc(Br)n2)cc1Cl]

The title compound was prepared and was isolated as a yellow solid (4.28 g, 47%): mp 172-178° C.; ¹H NMR (400 MHz, CDCl₃) δ 8.55 (s, 1H), 8.11 (d, J=8.9 Hz, 1H), 7.97 (d, J=2.3 Hz, 1H), 7.74 (dd, J=8.9, 2.4 Hz, 1H); ESIMS m/z 303 ([M+H]⁺).

### 4-Bromo-1-(3-chloro-4-nitrophenyl)-1H-imidazole (C489)

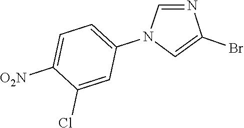 [SMILES: O=[N+]([O-])c1ccc(-n2cnc(Br)c2)cc1Cl]

The title compound was prepared and was isolated as a yellow solid (3.72 g, 89%): mp 160-164° C.; ¹H NMR (400 MHz, CDCl₃) δ 8.10 (d, J=8.8 Hz, 1H), 7.82 (d, J=1.7 Hz, 1H), 7.60 (d, J=2.4 Hz, 1H), 7.44 (dd, J=8.8, 2.4 Hz, 1H), 7.33 (d, J=1.6 Hz, 1H); ESIMS m/z 301 ([M−H]⁻).

### Example 94: Preparation of 4-(3-bromo-1H-1,2,4-triazol-1-yl)aniline (C490)

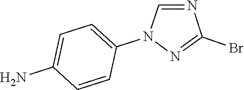 [SMILES: Nc1ccc(-n2cnc(Br)n2)cc1]

3-Bromo-1-(4-nitrophenyl)-1H-1,2,4-triazole (C486; 4.00 g, 14.9 mmol) was suspended in ethanol (59.5 mL) and water (14.87 mL), and to the resulting suspension were added ammonium chloride (3.18 g, 59.5 mmol) and iron (3.32 g, 59.5 mmol). The flask was outfitted with a reflux condenser and placed on a preheated plate set to 80° C. and allowed to stir at that temperature until LC-MS indicated complete product formation (0.5-2 h). The reaction mixture was cooled to room temperature, diluted with EtOAc, and filtered through a pad of diatomaceous earth, washing with EtOAc and water. The filtrate was transferred to a separatory funnel with the aid of water, and the layers were separated. The aqueous layer was extracted with EtOAc. The combined organic layers were washed with brine, dried over Na₂SO₄-magnesium sulfate, and concentrated in vacuo to afford the title compound as a beige solid (3.36 g, 94%), which required no further purification: ¹H NMR (400 MHz, CDCl₃) δ 8.26 (s, 1H), 7.40-7.34 (m, 2H), 6.79-6.71 (m, 2H), 3.88 (s, 2H); ESIMS m/z 239 ([M+H]⁺).

The following compounds were prepared in accordance to the procedure in Example 94:

### 2-Methyl-4-(3-(4-(trifluoromethoxy)phenyl)-1H-1,2,4-triazol-1-yl)aniline (C491)

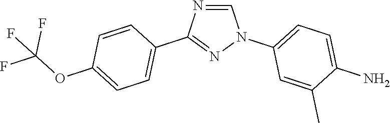 [SMILES: Cc1cc(-n2cnc(-c3ccc(OC(F)(F)F)cc3)n2)ccc1N]

The title compound was prepared and was isolated as a yellow solid (690 mg, 86%): ¹H NMR (400 MHz, CDCl₃) δ 8.41 (s, 1H), 8.26-8.18 (m, 2H), 7.43-7.38 (m, 1H), 7.34 (dd, J=8.4, 2.5 Hz, 1H), 7.33-7.26 (m, 2H), 6.76 (d, J=8.4 Hz, 1H), 3.79 (s, 2H), 2.25 (s, 3H); ¹⁹F NMR (376 MHz, CDCl₃) δ −57.71; ESIMS m/z 335.1 ([M+H]⁺).

### 4-(3-Bromo-1H-1,2,4-triazol-1-yl)-2-chloroaniline (C492)

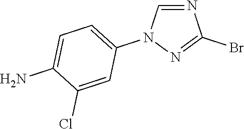 [SMILES: Nc1ccc(-n2cnc(Br)n2)cc1Cl]

The title compound was prepared and was isolated as an off-white solid (890 mg, 78%): mp 99-106° C.; ¹H NMR (400 MHz, CDCl₃) δ 8.27 (s, 1H), 7.56 (d, J=2.5 Hz, 1H), 7.31 (dd, J=8.6, 2.5 Hz, 1H), 6.84 (d, J=8.6 Hz, 1H), 4.27 (s, 2H); ESIMS m/z 273 ([M+H]⁺).

### 2-Fluoro-4-(5-(4-(trifluoromethoxy)phenyl)-1,2,4-oxadiazol-3-yl)aniline (C493)

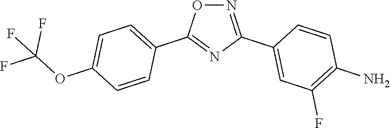 [SMILES: Nc1ccc(-c2noc(-c3ccc(OC(F)(F)F)cc3)n2)cc1F]

The title compound was prepared and was isolated as a pale yellow solid (70.4 mg, 13%): ¹H NMR (400 MHz, CDCl₃) δ 8.25 (d, J=8.9 Hz, 2H), 7.85-7.73 (m, 2H), 7.39 (d, J=8.3 Hz, 2H), 6.86 (t, J=8.7 Hz, 1H), 4.05 (s, 2H); ¹⁹F NMR (376 MHz, CDCl₃) δ −57.62, −135.03; ESIMS m/z 338 ([M−H]⁻).

### 2-Fluoro-4-(3-(4-(trifluoromethoxy)phenyl)-1,2,4-oxadiazol-5-yl)aniline (C494)

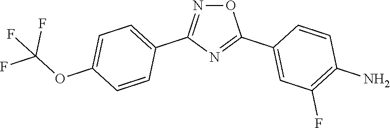 [SMILES: Nc1ccc(-c2nc(-c3ccc(OC(F)(F)F)cc3)no2)cc1F]

The title compound was prepared and was isolated as a yellow solid (218 mg, 23%): ¹H NMR (400 MHz, CDCl₃) δ 8.23-8.15 (m, 2H), 7.88-7.79 (m, 2H), 7.34 (d, J=8.3 Hz, 2H), 6.87 (t, J=8.6 Hz, 1H), 4.24 (s, 2H); ¹⁹F NMR (376 MHz, CDCl₃) δ −57.68, −134.79; ESIMS m/z 338 ([M−H]⁻).

### 2-Fluoro-4-(5-(4-(trifluoromethoxy)phenyl)-1,3,4-oxadiazol-2-yl)aniline (C495)

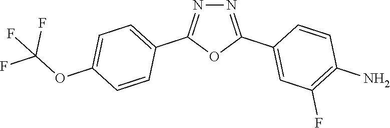 [SMILES: Nc1ccc(-c2nnc(-c3ccc(OC(F)(F)F)cc3)o2)cc1F]

The title compound was prepared and was isolated as a pale orange solid (569 mg, 61%): ¹H NMR (400 MHz, CDCl₃) δ 8.20-8.11 (m, 2H), 7.82-7.67 (m, 2H), 7.44-7.33 (m, 2H), 6.87 (t, J=8.6 Hz, 1H), 4.16 (s, 2H); ¹⁹F NMR (376 MHz, CDCl₃) δ −57.68, −134.61; ESIMS m/z 340 ([M+H]⁺).

### 2-Fluoro-4-(5-(4-(trifluoromethoxy)phenyl)-1,3,4-thiadiazol-2-yl)aniline (C496)

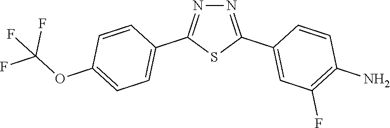 [SMILES: Nc1ccc(-c2nnc(-c3ccc(OC(F)(F)F)cc3)s2)cc1F]

The title compound was prepared and was isolated as a pale yellow solid (522 mg, 73%): ¹H NMR (400 MHz, CDCl₃) δ 8.07-7.99 (m, 2H), 7.72 (dd, J=11.7, 2.0 Hz, 1H), 7.61-7.54 (m, 1H), 7.34 (d, J=8.2 Hz, 2H), 6.84 (t, J=8.5 Hz, 1H), 4.10 (s, 2H); ¹⁹F NMR (376 MHz, CDCl₃) δ −57.71, −134.61; ESIMS m/z 356 ([M+H]⁺).

### 4-(1-(4-(Trifluoromethoxy)phenyl)-1H-1,2,3-triazol-4-yl)aniline (C497)

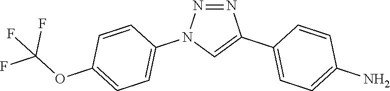 [SMILES: Nc1ccc(-c2cn(-c3ccc(OC(F)(F)F)cc3)nn2)cc1]

The title compound was prepared and was isolated as an off-white solid (709 mg, 85%): ¹H NMR (400 MHz, CDCl₃) δ 8.04 (s, 1H), 7.87-7.79 (m, 2H), 7.75-7.66 (m, 2H), 7.43-7.36 (m, 2H), 6.81-6.74 (m, 2H), 3.81 (s, 2H); ¹⁹F NMR (376 MHz, CDCl₃) δ −57.97; ESIMS m/z 321 ([M+H]⁺).

### 4-(2-(4-(Trifluoromethoxy)phenyl)-2H-tetrazol-5-yl)aniline (C498)

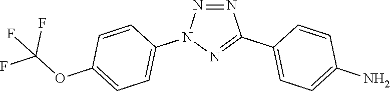 [SMILES: Nc1ccc(-c2nnn(-c3ccc(OC(F)(F)F)cc3)n2)cc1]

The title compound was prepared and was isolated as a beige solid (77.4 mg, 93%): ¹H NMR (400 MHz, CDCl₃) δ 8.28-8.19 (m, 2H), 8.11-8.00 (m, 2H), 7.46-7.38 (m, 2H), 6.83-6.74 (m, 2H), 3.96 (s, 2H); ¹⁹F NMR (376 MHz, CDCl₃) δ −57.92; ESIMS m/z 322 ([M+H]⁺).

### 4-(4-Bromo-1H-imidazol-1-yl)-2-chloroaniline (C499)

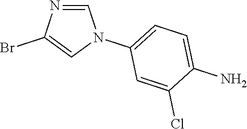 [SMILES: Nc1ccc(-n2cnc(Br)c2)cc1Cl]

The title compound was prepared and was isolated as a tan solid (2.20 g, 77%): ¹H NMR (400 MHz, CDCl₃) δ 7.57 (d, J=1.6 Hz, 1H), 7.27 (d, J=2.5 Hz, 1H), 7.13 (d, J=1.6 Hz, 1H), 7.06 (dd, J=8.5, 2.5 Hz, 1H), 6.82 (d, J=8.5 Hz, 1H), 4.23 (s, 2H); ESIMS m/z 274 ([M+H]⁺).

### Example 95: Preparation of 3-bromo-1-(4-isothiocyanatophenyl)-1H-1,2,4-triazole (C500)

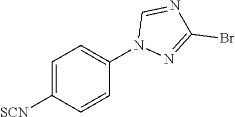

To a solution of 4-(3-bromo-1H-1,2,4-triazol-1-yl)aniline (C490; 2 g, 8.36 mmol) in benzene-THF (2:1 ratio; 30 mL), was added carbon disulfide (5 mL, 83.6 mmol) at 0° C. Triethylamine (12 mL, 83.6 mmol) was added dropwise, and the reaction mixture was allowed to warm to room temperature and stirred for 16 h. The obtained carbamate salt intermediate was isolated by filtration and dried under vacuum. The residue was dissolved in chloroform (20 mL), and ethyl chloroformate (1.29 mL, 8.36 mmol) was added slowly at 0° C. The reaction mixture was stirred at the same temperature for 30 minutes. The reaction mixture was quenched with ice water (10 mL) and was extracted with DCM (2×10 mL). The organic layer was washed with water (10 mL) and brine (10 mL), dried over anhydrous Na₂SO₄, filtered, and concentrated under reduced pressure to afford the title compound as an off-white solid (1.5 g, 64%): ¹H NMR (400 MHz, CDCl₃) δ 8.42 (s, 1H), 7.68-7.64 (m, 2H), 7.38-7.35 (m, 2H); ESIMS m/z 281 ([M+H]⁺).

### Example 96: Preparation of (4-(3-bromo-1H-1,2,4-triazol-1-yl)phenyl)(trifluoromethyl)carbamic fluoride (C501)

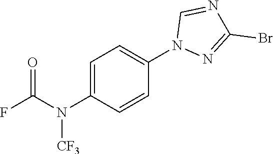 [SMILES: O=C(F)N(c1ccc(-n2cnc(Br)n2)cc1)C(F)(F)F]

To a sealed tube containing 3-bromo-1-(4-isothiocyanatophenyl)-1H-1,2,4-triazole (C500; 1 g, 3.55 mmol) in acetonitrile (30 mL) were added silver(I) fluoride (2.48 g, 19.5 mmol) and triphosgene (0.42 g, 1.42 mmol) quickly at 0° C. The reaction mixture was allowed to warm to room temperature and was stirred for 16 h. The reaction mixture was diluted with diethyl ether (20 mL) and was stirred for 10 minute. The obtained solids were filtered on a diatomaceous earth pad, and the filtrate was concentrated under reduced pressure. The resulting material was taken up in diethyl ether (20 mL) again. The mixture was washed with water (10 mL), dried over anhydrous Na₂SO₄, filtered and concentrated under reduced pressure. The title compound was isolated as a grey solid (0.75 g, 79%): mp 133-137° C.; ¹H NMR (400 MHz, CDCl₃) δ 8.50 (s, 1H), 7.82 (dd, J=2.4, 6.8 Hz, 2H), 7.52-7.51 (m, 2H); ¹⁹F NMR (376 MHz, CDCl₃) δ −4.93, −54.88; ESIMS m/z 353 ([M+H]⁺).

### Example 97: Preparation of methyl (4-(3-bromo-1H-1,2,4-triazol-1-yl)phenyl)(trifluoromethyl)carbamate (C502)

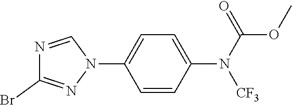 [SMILES: COC(=O)N(c1ccc(-n2cnc(Br)n2)cc1)C(F)(F)F]

To a solution of (4-(3-bromo-1H-1,2,4-triazol-1-yl)phenyl)(trifluoromethyl)carbamic fluoride (C501; 0.6 g, 1.69 mmol) in methanol (30 mL), potassium carbonate (1.1 equivalents) was added and was stirred at room temperature for 4 h. The reaction mixture was concentrated under reduced pressure. Purification of the resulting material by silica gel column chromatography (silica gel 100-200 mesh) eluting with 30-35% EtOAc in petroleum ether afforded the title compound as an off white solid (0.42 g, 68%): FT-IR 1753 cm⁻¹ (C═O stretching present); mp 88-92° C.; ¹H NMR (400 MHz, CDCl₃) δ 8.47 (s, 1H), 7.75-7.73 (m, 2H), 7.44-7.42 (m, 2H), 3.81 (s, 3H); ¹⁹F NMR (376 MHz, CDCl₃) δ −54.54; ESIMS m/z 365 ([M+H]⁺).

### Example 98: Preparation of (Z)-N′-hydroxy-4-(trifluoromethoxy)benzimidamide (C503)

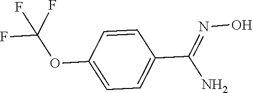 [SMILES: N/C(=N\O)c1ccc(OC(F)(F)F)cc1]

4-(Trifluoromethoxy)benzonitrile (2.34 mL, 16.0 mmol) was dissolved in ethanol (53.4 mL), and to the resulting solution was added hydroxylamine (1.47 mL, 24.1 mmol). The reaction mixture was warmed to 100° C. and stirred overnight. After stirring 17 h, LC-MS indicated reaction completion. The reaction mixture was allowed to cool to room temperature, and the ethanol was removed in vacuo, yielding a white solid. The solid was taken up in EtOAc and transferred to a separatory funnel with the aid of brine. The layers were separated, and the organic layer was passed through a phase separator and concentrated in vacuo to afford a white solid (with some oily residues), which was triturated with heptanes and collected by vacuum filtration. The title compound was isolated as a white solid (3.16 g, 89%), which required no further purification: ¹H NMR (400 MHz, CDCl₃) δ 7.69-7.64 (m, 2H), 7.28-7.22 (m, 2H), 7.11 (s, 1H), 4.84 (s, 2H); ¹⁹F NMR (376 MHz, CDCl₃) δ −57.80; ESIMS m/z 221 ([M+H]⁺).

The following compound was prepared in accordance to the procedure in Example 98:

### (Z)-3-Fluoro-N′-hydroxy-4-nitrobenzimidamide (C504)

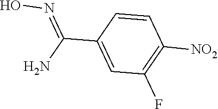 [SMILES: N/C(=N\O)c1ccc([N+](=O)[O-])c(F)c1]

The title compound was prepared and was isolated as an orange solid (4.47 g, 71%): ¹H NMR (400 MHz, CDCl₃) δ 8.11 (dd, J=8.5, 7.4 Hz, 1H), 7.68-7.49 (m, 2H), 6.72 (s, 1H), 4.85 (s, 2H); ¹⁹F NMR (376 MHz, CDCl₃) δ −116.04; ESIMS m/z 200 ([M+H]⁺).

### Example 99: Preparation of (Z)-3-fluoro-4-nitro-N′-((4-(trifluoromethoxy)benzoyl)oxy)benzimidamide (C505)

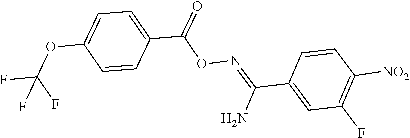 [SMILES: N/C(=N\OC(=O)c1ccc(OC(F)(F)F)cc1)c1ccc([N+](=O)[O-])c(F)c1]

(Z)-3-Fluoro-N′-hydroxy-4-nitrobenzimidamide (C504; 2.00 g, 10.0 mmol) was suspended in DCM (50.2 mL), and triethylamine (1.54 mL, 11.1 mmol) was added at room temperature The dark orange suspension became more clear, but still had solids suspended in the solvent. The reaction mixture was cooled to 0° C., and 4-(trifluoromethoxy)benzoyl chloride (1.58 mL, 10.0 mmol) was added dropwise, causing the solution to progressively become completely clear, yet did not change color. The reaction mixture was warmed to room temperature and allowed to stir overnight. After stirring for 17 h, LC-MS indicated reaction completion. The solvent was removed in vacuo, forming a dark red oil. This residue was taken up in water-heptanes, causing a tan solid to precipitate. The solid was collected by vacuum filtration and washed with hexanes. A solid began to precipitate from the filtrate. The filtrate was transferred to a separatory funnel and was extracted with EtOAc. The organic layer was passed through a phase separator and concentrated, and the residual orange oil was taken up in the minimal amount of EtOAc. Heptanes was added to precipitate the solid. The resulting solids were collected by vacuum filtration. This process was repeated one more time. The three batches of solids collected were dried under high vacuum overnight. The title compound was isolated as a dark yellow solid (3.33 g, 81%), which required no further purification: ¹H NMR (400 MHz, CDCl₃) δ 8.19-8.11 (m, 3H), 7.78 (dd, J=11.0, 1.8 Hz, 1H), 7.73 (dd, J=8.5, 1.8 Hz, 1H), 7.34 (d, J=8.1 Hz, 2H), 5.21 (s, 2H); ¹⁹F NMR (376 MHz, CDCl₃) δ −57.32, −115.35; ESIMS m/z 388 ([M+H]⁺).

### Example 100: Preparation of 3-(3-fluoro-4-nitrophenyl)-5-(4-(trifluoromethoxy)phenyl)-1,2,4-oxadiazole (C506)

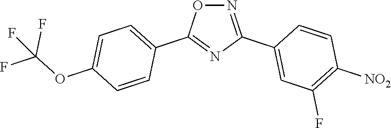 [SMILES: O=[N+]([O-])c1ccc(-c2noc(-c3ccc(OC(F)(F)F)cc3)n2)cc1F]

(Z)-3-Fluoro-4-nitro-N′-((4-(trifluoromethoxy)benzoyl)oxy)benzimidamide (C505; 1.00 g, 2.58 mmol) was dissolved in THF (20 mL), and to the resulting solution was added tetrabutylammonium hydroxide (TBAH; 0.168 mL, 0.258 mmol) dropwise. No discernible color change was observed. After stirring at room temperature for 2 h, little progress was observed by TLC. Additional TBAH (2.32 mmol) was added, and the reaction mixture was allowed to stir overnight. After stirring for 17 h, LC-MS indicated reaction completion. The reaction mixture was concentrated in vacuo to afford a dark red solid. Purification by normal-phase high-performance liquid chromatography eluting with 0-30% EtOAc-hexanes afforded the title compound as an off-white solid (624 mg, 65%): ¹H NMR (400 MHz, CDCl₃) δ 8.28 (d, J=8.4 Hz, 2H), 8.22 (t, J=8.0 Hz, 1H), 8.17-8.10 (m, 2H), 7.43 (d, J=8.4 Hz, 2H); ¹⁹F NMR (376 MHz, CDCl₃) δ −58.14, −116.28; ESIMS m/z 370 ([M+H]⁺).

### Example 101: Preparation of 5-(3-fluoro-4-nitrophenyl)-3-(4-(trifluoromethoxy)phenyl)-1,2,4-oxadiazole (C507)

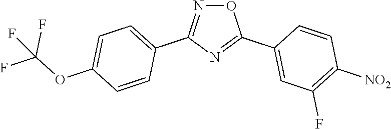 [SMILES: O=[N+]([O-])c1ccc(-c2nc(-c3ccc(OC(F)(F)F)cc3)no2)cc1F]

3-Fluoro-4-nitrobenzoic acid (1.68 g, 9.08 mmol) and (Z)-N′-hydroxy-4-(trifluoromethoxy)benzimidamide (C503; 2.00 g, 9.08 mmol) were dissolved in EtOAc (45.4 mL), and to the resulting solution was added triethylamine (3.80 mL, 27.3 mmol). The reaction mixture became darker yellow. At this time 2,4,6-tripropyl-1,3,5,2,4,6-trioxatriphosphinane-2,4,6-trioxide (13.5 mL, 22.7 mmol) was added, and the reaction mixture was allowed to stir at 80° C. overnight. After stirring for 18 h, LC-MS indicated reaction completion and clean product formation. The solvent was removed in vacuo, and the residue was stirred rapidly and diluted with water, causing a tan solid to precipitate. The solid was collected by vacuum filtration, immediately re-suspended in water and stirred vigorously for 30 minutes. The solid was collected by vacuum filtration and dried under high vacuum to afford the title compound as a tan solid (2.68 g, 79%): ¹H NMR (400 MHz, CDCl₃) δ 8.31-8.13 (m, 5H), 7.42-7.34 (m, 2H); ¹⁹F NMR (376 MHz, CDCl₃) δ −57.67, −114.83; ESIMS m/z 370 ([M+H]⁺).

### Example 102: Preparation of 3-fluoro-4-nitro-N′-(4-(trifluoromethoxy)benzoyl)benzohydrazide (C508)

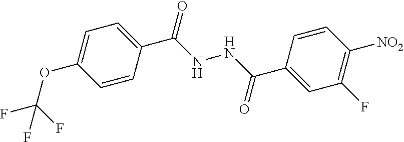 [SMILES: O=C(NNC(=O)c1ccc([N+](=O)[O-])c(F)c1)c1ccc(OC(F)(F)F)cc1]

3-Fluoro-4-nitrobenzoic acid (1.68 g, 9.08 mmol) and 4-(trifluoromethoxy)benzohydrazide (2.00 g, 9.08 mmol) were dissolved in EtOAc (45.4 mL), and to the resulting solution was added pyridine (1.62 mL, 20.0 mmol). At this time, the reaction became a cloudy beige. 2,4,6-Tripropyl-1,3,5,2,4,6-trioxatriphosphinane-2,4,6-trioxide (11.9 mL, 20.0 mmol) was added, causing the reaction mixture to revert back to clear tan. The reaction mixture was allowed to stir at room temperature overnight. After stirring for 17 h, LC-MS indicated reaction completion. The reaction mixture was concentrated in vacuo, and the residue was stirred vigorously and diluted with water, causing a white solid to precipitate. The solid was collected by vacuum filtration, was immediately re-suspended in water, and was stirred vigorously for another 30 minutes. The solid was collected by vacuum filtration and dried under high vacuum to afford the title compound as a white solid (3.33 g, 94%): ¹H NMR (400 MHz, CDCl₃) δ 9.31 (s, 1H), 9.07 (s, 1H), 8.18 (t, J=7.8 Hz, 1H), 7.93 (d, J=8.3 Hz, 2H), 7.85 (d, J=10.6 Hz, 1H), 7.78 (d, J=8.3 Hz, 1H), 7.35 (d, J=8.3 Hz, 2H); ¹⁹F NMR (376 MHz, CDCl₃) δ −58.21, −115.43; ESIMS m/z 388 ([M+H]⁺).

### Example 103: Preparation of 2-(3-fluoro-4-nitrophenyl)-5-(4-(trifluoromethoxy)phenyl)-1,3,4-oxadiazole (C509)

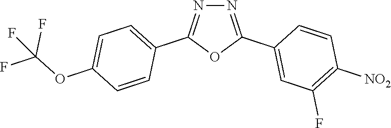 [SMILES: O=[N+]([O-])c1ccc(-c2nnc(-c3ccc(OC(F)(F)F)cc3)o2)cc1F]

3-Fluoro-4-nitro-N′-(4-(trifluoromethoxy)benzoyl)benzohydrazide (C508; 1.25 g, 3.23 mmol) was taken up in EtOAc (16 mL). Triethylamine (1.35 mL, 9.68 mmol), followed by 2,4,6-tripropyl-1,3,5,2,4,6-trioxatriphosphinane-2,4,6-trioxide (4.80 mL, 8.07 mmol) were added. The reaction mixture was allowed to stir at 80° C. overnight. After stirring for 17 h, LC-MS indicated reaction completion. The reaction mixture was allowed to cool to room temperature and was concentrated in vacuo. The residue was stirred vigorously and diluted with water, causing a tan solid to precipitate. This solid was collected by vacuum filtration, was immediately re-suspended in water, and was stirred vigorously for another 30 minutes. The solid was collected by vacuum filtration and dried under high vacuum overnight to afford the title compound as a grey solid (1.09 g, 91%): ¹H NMR (400 MHz, CDCl₃) δ 8.29-8.23 (m, 1H), 8.22 (d, J=8.8 Hz, 2H), 8.10 (s, 1H), 8.09-8.06 (m, 1H), 7.42 (d, J=8.4 Hz, 2H); ¹⁹F NMR (376 MHz, CDCl₃) δ −57.65, −114.81; ESIMS m/z 370 ([M+H]⁺).

### Example 104: Preparation of 2-(3-fluoro-4-nitrophenyl)-5-(4-(trifluoromethoxy)phenyl)-1,3,4-thiadiazole (C510)

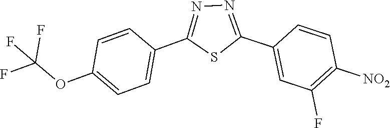 [SMILES: O=[N+]([O-])c1ccc(-c2nnc(-c3ccc(OC(F)(F)F)cc3)s2)cc1F]

To a solution of 3-fluoro-4-nitro-N′-(4-(trifluoromethoxy)benzoyl)benzohydrazide (C508; 2.00 g, 5.16 mmol) in EtOAc (51.6 mL) were added Lawesson's reagent (3.13 g, 7.75 mmol) and triethylamine (1.80 mL, 12.9 mmol). 2,4,6-Tripropyl-1,3,5,2,4,6-trioxatriphosphinane-2,4,6-trioxide (3.69 mL, 6.20 mmol) was added dropwise. The reaction mixture was warmed to 60° C. and allowed to stir overnight. After stirring 17 h, the reaction mixture was allowed to cool to room temperature and the EtOAc was removed in vacuo. The residue was taken up in water and DCM and transferred to a separatory funnel with the aid of saturated aqueous sodium bicarbonate. The layers were separated (some solids remained suspended in the organic layer), and the aqueous layer was extracted with DCM. The combined organic layers were washed with brine, but this caused an emulsion to form, which was not easily broken up by the addition of more saturated aqueous sodium bicarbonate. The biphasic layer was filtered over a pad of diatomaceous earth, which successfully removed the emulsion. The layers were separated again, and the aqueous layer was extracted with DCM. The combined organic layers were passed through a phase separator and concentrated in vacuo to afford an orange oil. The residue was purified by normal-phase high-performance liquid chromatography, eluting with 0-30% EtOAc-hexanes to afford the title compound as a pale yellow solid (718 mg, 33%): ¹H NMR (400 MHz, CDCl₃) δ 8.22 (ddd, J=8.5, 6.5, 1.6 Hz, 1H), 8.13-8.06 (m, 2H), 8.02 (dd, J=11.1, 1.8 Hz, 1H), 7.95-7.90 (m, 1H), 7.42-7.35 (m, 2H); ¹⁹F NMR (376 MHz, CDCl₃) δ −57.68, −114.99; ESIMS m/z 386 ([M+H]⁺).

### Example 105: Preparation of 4-(4-nitrophenyl)-1-(4-(trifluoromethoxy)phenyl)-1H-1,2,3-triazole (C511)

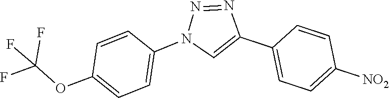 [SMILES: O=[N+]([O-])c1ccc(-c2cn(-c3ccc(OC(F)(F)F)cc3)nn2)cc1]

1-(4-Nitrophenyl)ethan-1-one (2 g, 12.1 mmol), 4-(trifluoromethoxy)aniline (1.95 mL, 14.5 mmol), and 4-methylbenzenesulfonohydrazide (3.38 g, 18.2 mmol) were dissolved in DMSO (50 mL). Molecular iodine (4.61 g, 18.2 mmol) was added, and the reaction mixture was warmed to 100° C. and allowed to stir at this temperature for 72 h. The reaction mixture was allowed to cool to room temperature (a solid formed), was diluted with a large amount of water, and was transferred to a separatory funnel with the aid of a large amount of EtOAc. The layers were separated, and the aqueous layer was extracted twice with EtOAc. The combined organic layers were washed with saturated sodium bisulfite to remove excess iodine, were passed through a phase separator, and were concentrated in vacuo to form a brown solid. This residue was purified by normal-phase high-performance liquid chromatography, eluting with 0-30% EtOAc-hexanes to afford the title compound as a yellow solid (921 mg, 22%): mp 170-173° C.; ¹H NMR (400 MHz, CDCl₃) δ 8.35 (s, 1H), 8.33 (d, J=1.6 Hz, 2H), 8.13-8.06 (m, 2H), 7.86 (d, J=9.0 Hz, 2H), 7.44 (d, J=8.5 Hz, 2H); ESIMS m/z 351 ([M+H]⁺).

### Example 106: Preparation of 5-(4-nitrophenyl)-2-(4-(trifluoromethoxy)phenyl)-2H-tetrazole (C512)

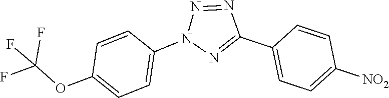 [SMILES: O=[N+]([O-])c1ccc(-c2nnn(-c3ccc(OC(F)(F)F)cc3)n2)cc1]

5-(4-Nitrophenyl)-2H-tetrazole (500 mg, 2.62 mmol) was taken up in DCM (13 mL), and (4-(trifluoromethoxy)phenyl)boronic acid (1077 mg, 5.23 mmol), copper(II) acetate (950 mg, 5.23 mmol) and pyridine (421 μl, 5.23 mmol) were added. The reaction mixture was allowed to stir under air at room temperature overnight. After stirring for 17 h, the reaction mixture was diluted with DCM and was passed through a plug of diatomaceous earth, washing with DCM. The green-blue filtrate was diluted with 5% aqueous ammonium hydroxide, forming a deep blue aqueous layer. This biphasic mixture was transferred to a separatory funnel, and the layers were separated. The aqueous layer was extracted twice with DCM. The combined organic layers were washed with brine, were passed through a phase separator, and were concentrated in vacuo to afford a brown oil, which eventually crystallized into a brown solid. The residue was purified by normal-phase high-performance liquid chromatography, eluting with 0-20% EtOAc-hexanes to afford the title compound as a white solid (93.8 mg, 10%): ¹H NMR (400 MHz, CDCl₃) δ 8.51-8.38 (m, 4H), 8.33-8.25 (m, 2H), 7.47 (dq, J=7.9, 1.0 Hz, 2H); ¹⁹F NMR (376 MHz, CDCl₃) δ −57.90; ESIMS m/z 352 ([M+H]⁺).

### Example 107: Preparation of 2-fluoro-4-(1-(6-(trifluoromethyl)pyridazin-3-yl)-1H-pyrazol-3-yl)aniline (C513)

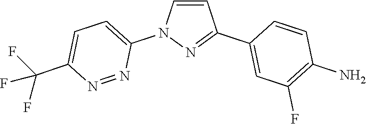 [SMILES: Nc1ccc(-c2ccn(-c3ccc(C(F)(F)F)nn3)n2)cc1F]

To a solution of compound 3-(3-bromo-1H-pyrazol-1-yl)-6-(trifluoromethyl)pyridazine (C457; 680 mg, 2.32 mmol) and 2-fluoro-4-(4,4,5,5-tetramethyl-1,3,2-dioxaborolan-2-yl)aniline (660 mg, 2.78 mmol) in DMA-water (20 mL:8 mL) was added potassium phosphate tribasic (1.7 g, 8.12 mmol). The mixture was stirred and degassed with nitrogen for 15 minutes. Tetrakis(triphenylphosphine)palladium(0) (134 mg, 0.116 mmol) was added under nitrogen. The mixture was heated at 100° C. for 2 h. The reaction mixture was cooled to room temperature and passed through a diatomaceous earth pad using EtOAc (20 mL). The organic layer was washed with water and brine, dried over Na₂SO₄, and concentrated under reduced pressure. Purification of the residue by flash silica gel chromatography eluting with 0-50% EtOAc in hexane yielded the title compound as a pale yellow solid (236 mg, 20%): ¹H NMR (DMSO-d₆) δ 8.90 (d, J=2.5 Hz, 1H), 8.48 (d, J=9.2 Hz, 1H), 8.40 (d, J=9.2 Hz, 1H), 7.66 (d, J=12.7 Hz, 1H), 7.57 (d, J=8.2 Hz, 1H), 7.14 (s, 1H), 6.86 (t, J=8.8 Hz, 1H), 5.51 (s, 2H); ESIMS m/z 324 ([M+H]⁺).

### Example 108: Preparation of 2-fluoro-4-(1-(6-(trifluoromethyl)pyridazin-3-yl)-1H-1,2,4-triazol-3-yl)aniline (C514)

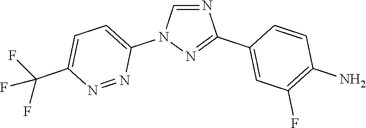 [SMILES: Nc1ccc(-c2ncn(-c3ccc(C(F)(F)F)nn3)n2)cc1F]

To a solution of compound 3-(3-bromo-1H-1,2,4-triazol-1-yl)-6-(trifluoromethyl)pyridazine (C458; 1 g, 3.4 mmol) and 2-fluoro-4-(4,4,5,5-tetramethyl-1,3,2-dioxaborolan-2-yl)aniline (1.2 g, 5.1 mmol) in acetonitrile-water (25 mL:13 mL) was added potassium fluoride (593 mg, 10.2 mmol). The mixture was stirred and degassed with nitrogen for 15 minutes. Tetrakis(triphenylphosphine)palladium(0) (786 mg, 0.68 mmol) was added under nitrogen. The mixture was heated at 80° C. overnight. The reaction mixture was cooled to room temperature and passed through a diatomaceous earth pad using EtOAc (50 mL). The organic layer was washed with water and brine, dried over Na₂SO₄, and concentrated under reduced pressure. Purification of the residue by flash silica gel chromatography eluting with 0-30% EtOAc in hexane yielded the title compound as a pale yellow solid (228 mg, 20%): ¹H NMR (DMSO-d₆): δ 9.72 (s, 1H), 8.54 (d, J=9.2 Hz, 1H), 8.46 (d, J=9.1 Hz, 1H), 7.78, 7.65 (m, 2H), 6.91 (d, J=9.2 Hz, 1H), 5.70 (s, 2H); ESIMS m/z 325 ([M+H]⁺).

### Example 109: Preparation of 1-(4-(methoxymethoxy)phenyl)-3-(3-methyl-4-nitrophenyl)-1H-1,2,4-triazole (C515)

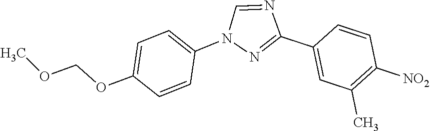 [SMILES: COCOc1ccc(-n2cnc(-c3ccc([N+](=O)[O-])c(C)c3)n2)cc1]

3-Bromo-1-(4-(methoxymethoxy)phenyl)-1H-1,2,4-triazole (C444; 1.2 g, 4.22 mmol), 4,4,5,5-tetramethyl-2-(3-methyl-4-nitrophenyl)-1,3,2-dioxaborolane (1.33 g, 5.07 mmol), potassium phosphate tribasic (2.69 g, 12.67 mmol) and XPhos-Pd-G3 precatalyst (0.072 g, 0.084 mmol) were combined in a 10:1 mixture of dioxane (12.8 mL) and water (1.28 mL). The mixture was heated to 100° C. for 8 h. The mixture was cooled and partitioned between DCM and brine. Separation and concentration of the organic solvents afforded material that was purified on silica gel eluting with 0-30% acetone-hexanes. The title compound was isolated as an orange waxy solid (1.00 g, 70%): ¹H NMR (400 MHz, CDCl₃) δ 8.52 (s, 1H), 8.20 (d, J=1.8 Hz, 1H), 8.16 (dd, J=8.5, 1.9 Hz, 1H), 8.09 (d, J=8.5 Hz, 1H), 7.69-7.62 (m, 2H), 7.22-7.17 (m, 2H), 5.24 (s, 2H), 3.52 (s, 3H), 2.70 (s, 3H); ESIMS m/z 341.0 ([M+H]⁺).

The following compound was prepared in accordance to the procedure in Example 109:

### 3-(3-Fluoro-4-nitrophenyl)-1-(4-(methoxymethoxy)phenyl)-1H-1,2,4-triazole (C516)

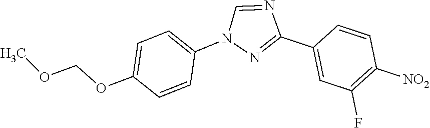 [SMILES: COCOc1ccc(-n2cnc(-c3ccc([N+](=O)[O-])c(F)c3)n2)cc1]

The title compound was prepared and was isolated as a brown solid (0.394 g, 43%): ¹H NMR (400 MHz, CDCl₃) δ 8.53 (s, 1H), 8.20-8.10 (m, 3H), 7.67-7.62 (m, 2H), 7.23-7.18 (m, 2H), 5.24 (s, 2H), 3.52 (s, 3H); ¹⁹F NMR (376 MHz, CDCl₃) δ −116.58.

### 1-(2-Amino-5-(1-(4-(trifluoromethoxy)phenyl)-1H-1,2,4-triazol-3-yl)phenyl)ethan-1-one (C517)

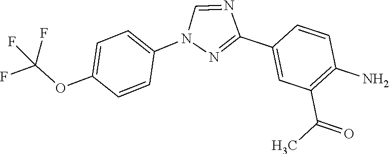 [SMILES: CC(=O)c1cc(-c2ncn(-c3ccc(OC(F)(F)F)cc3)n2)ccc1N]

The title compound was prepared and was isolated as a yellow solid (0.400 g, 34%): ¹H NMR (400 MHz, CDCl₃) δ 8.57 (d, J=2.0 Hz, 1H), 8.53 (s, 1H), 8.10 (dd, J=8.6, 2.0 Hz, 1H), 7.83-7.77 (m, 2H), 7.41-7.36 (m, 2H), 6.75 (d, J=8.6 Hz, 1H), 6.53 (s, 2H), 2.71 (s, 3H); ¹⁹F NMR (376 MHz, CDCl₃) δ −58.02.

### 1-(2-Amino-5-(1-(5-(trifluoromethyl)pyridin-2-yl)-1H-1,2,4-triazol-3-yl)phenyl)ethan-1-one (C518)

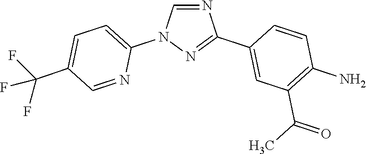 [SMILES: CC(=O)c1cc(-c2ncn(-c3ccc(C(F)(F)F)cn3)n2)ccc1N]

The title compound was prepared and was isolated as a yellow solid (0.420 g, 59%): ¹H NMR (400 MHz, CDCl₃) δ 9.22 (s, 1H), 8.74 (s, 1H), 8.60 (d, J=2.0 Hz, 1H), 8.16-8.08 (m, 3H), 6.76 (d, J=8.7 Hz, 1H), 6.57 (s, 2H), 2.72 (s, 3H); ¹⁹F NMR (376 MHz, CDCl₃) δ −62.05.

### 2-(4-(3-(4-Amino-3-fluorophenyl)-1H-1,2,4-triazol-1-yl)phenyl)acetonitrile (C519)

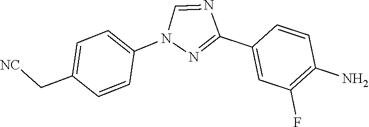 [SMILES: N#CCc1ccc(-n2cnc(-c3ccc(N)c(F)c3)n2)cc1]

The title compound was prepared using sodium carbonate and was isolated as an off-white powder (0.050 g, 8.5%): ¹H NMR (400 MHz, CDCl₃) δ 8.54 (s, 1H), 7.85-7.79 (m, 2H), 7.77 (dd, J=8.6, 2.1 Hz, 2H), 7.52-7.46 (m, 2H), 6.85 (dd, J=9.0, 8.2 Hz, 1H), 3.92 (s, 2H), 3.83 (s, 2H); ¹⁹F NMR (376 MHz, CDCl₃) δ −135.38.

### Example 110: Preparation of 2-(4-(3-(3-methyl-4-nitrophenyl)-1H-1,2,4-triazol-1-yl)phenoxy)acetonitrile (C520)

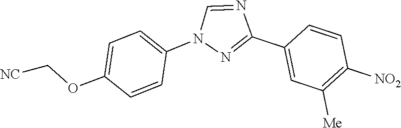 [SMILES: Cc1cc(-c2ncn(-c3ccc(OCC#N)cc3)n2)ccc1[N+](=O)[O-]]

1-(4-(Methoxymethoxy)phenyl)-3-(3-methyl-4-nitrophenyl)-1H-1,2,4-triazole (C515; 1 g, 2.94 mmol) was dissolved in THF (14.7 mL) and to the mixture was added was hydrochloric acid (10% aq; 0.893 mL, 2.94 mmol). The mixture was heated to 50° C. and stirred for 16 h. The mixture was cooled and the solvent was removed. Dichloromethane was added, and the mixture was treated with triethylamine. After washing with water, the layers were separated and the DCM was concentrated. The resulting yellow solid was used without further purification. 4-(3-(3-Methyl-4-nitrophenyl)-1H-1,2,4-triazol-1-yl)phenol (material isolated from the previous reaction; 0.973 g, 3.28 mmol) was suspended in acetone (13.1 mL). Potassium carbonate (1.362 g, 9.85 mmol) and bromoacetonitrile (0.343 mL, 4.93 mmol) were added. The mixture was heated to 65° C. and stirred for 8 h. The mixture was filtered and the solvent was removed. Purification by column chromatography eluting with 0-100% acetone-hexanes. The title compound was isolated as a tan solid (1.04 g, 94%): ¹H NMR (400 MHz, CDCl₃) δ 8.55 (s, 1H), 8.20 (s, 1H), 8.16 (dd, J=8.4, 1.9 Hz, 1H), 8.10 (d, J=8.5 Hz, 1H), 7.78-7.73 (m, 2H), 7.19-7.14 (m, 2H), 4.86 (s, 2H), 2.71 (s, 3H); ESIMS m/z 336 ([M+H]⁺).

The following compound was prepared in accordance to the procedure in Example 110:

### 2-(4-(3-(3-Fluoro-4-nitrophenyl)-1H-1,2,4-triazol-1-yl)phenoxy)acetonitrile (C521)

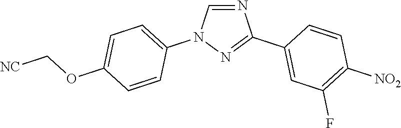 [SMILES: N#CCOc1ccc(-n2cnc(-c3ccc([N+](=O)[O-])c(F)c3)n2)cc1]

The title compound was prepared and was isolated as a tan solid (0.340 g, 84%): ¹H NMR (400 MHz, CDCl₃) δ 9.17 (s, 1H), 8.34-8.28 (m, 1H), 8.24-8.19 (m, 1H), 8.16 (dd, J=12.1, 1.6 Hz, 1H), 8.02-7.97 (m, 2H), 7.37-7.31 (m, 2H), 5.24 (s, 2H), 2.78 (s, 3H); ¹⁹F NMR (376 MHz, CDCl₃) δ −113.99.

### Example 111: Preparation of 2-(4-(3-(4-amino-3-fluorophenyl)-1H-1,2,4-triazol-1-yl)phenoxy)acetonitrile (C522)

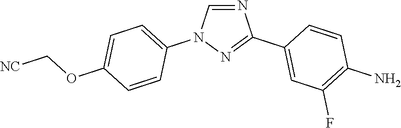 [SMILES: N#CCOc1ccc(-n2cnc(-c3ccc(N)c(F)c3)n2)cc1]

2-(4-(3-(3-Fluoro-4-nitrophenyl)-1H-1,2,4-triazol-1-yl)phenoxy)acetonitrile (C521; 0.340 g, 1.00 mmol) was dissolved in ethanol (3.34 mL) and water (1.67 mL). Iron powder (0.280 g, 5.01 mmol) and solid ammonium chloride (0.027 g, 0.501 mmol were added sequentially. The mixture was heated to 80° C. for 3 h. The mixture was filtered through diatomaceous earth and the solvents were removed. Residual water was removed by azeotrope with toluene. The resulting brown solid was used without further purification (0.280 g, 90%): ESIMS m/z 310 ([M+H]⁺).

The following compound was prepared in accordance to the procedure in Example 111:

### 2-(4-(3-(4-Amino-3-methylphenyl)-1H-1,2,4-triazol-1-yl)phenoxy)acetonitrile (C523)

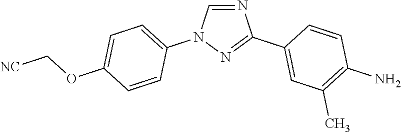 [SMILES: Cc1cc(-c2ncn(-c3ccc(OCC#N)cc3)n2)ccc1N]

The title compound was prepared and was isolated as a yellow powder (0.340 g, 75%).

### Example 112: Preparation of 4-(1-(3-(trifluoromethyl)phenyl)-1H-1,2,4-triazol-3-yl)aniline (C524)

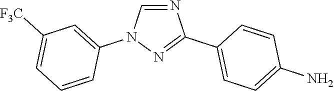 [SMILES: Nc1ccc(-c2ncn(-c3cccc(C(F)(F)F)c3)n2)cc1]

A solution of 3-bromo-1-(3-(trifluoromethyl)phenyl)-1H-1,2,4-triazole (C45; 2.5 g, 8.59 mmol) in 1,4-dioxane-water (3:1; 16 mL) was degassed with argon. 4-(4,4,5,5-Tetramethyl-1,3,2-dioxaborolan-2-yl)aniline (2.1 g, 9.45 mmol), potassium carbonate (1.8 g, 12.9 mmol), and Pd(PPh₃)₄ (1.0 g, 0.85 mmol) were added, and the reaction mixture was stirred at 110° C. for 16 h. The reaction mixture was cooled to room temperature, diluted with EtOAc (20 mL), and washed with water. The aqueous layer was extracted with EtOAc (2×10 mL). The organic layers were washed with brine, dried over anhydrous Na₂SO₄, filtered, and concentrated under reduced pressure. Purification by column chromatography (30-50% EtOAc-petroleum ether) provided the title compound as a pale brown solid (1.5 g, 58%): mp 128-130° C.; ¹H NMR (400 MHz, DMSO-d₆) δ 9.36 (s, 1H), 8.24-8.20 (m, 2H), 7.81-7.71 (m, 4H), 6.63 (d, J=8.4 Hz, 2H), 5.48 (br s, 2H); ¹³C NMR (101 MHz, DMSO-d₆) δ 163.02, 150.44, 143.35, 137.47, 131.18, 127.48, 125.14, 123.68, 122.70, 117.39, 115.33, 113.63; ¹⁹F NMR (376 MHz, DMSO-d₆) δ−61.23; ESIMS m/z 305 ([M+H]⁺).

The following compound was prepared according to the procedure described in Example 112:

### 4-(1-(3-(Trifluoromethoxy)phenyl)-1H-1,2,4-triazol-3-yl)aniline (C525)

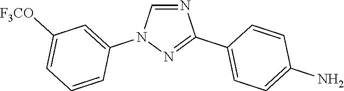 [SMILES: Nc1ccc(-c2ncn(-c3cccc(OC(F)(F)F)c3)n2)cc1]

The title compound was prepared and was isolated as an off-white solid (1.6 g, 52%): mp 119-124° C.; ¹H NMR (400 MHz, DMSO-d₆) δ 9.33 (s, 1H) 7.99-7.94 (m, 2H), 7.79 (d, J=8.8 Hz, 2H), 7.70 (t, J=8.4 Hz, 1H), 7.40 (d, J=8.4 Hz, 1H), 6.66 (d, J=9.2 Hz, 2H), 5.49 (s, 2H); ¹³C NMR (100 MHz, DMSO-d₆) δ 162.88, 150.31, 148.93, 143.18, 138.16, 131.63, 127.34, 119.18, 117.58, 117.33, 113.50, 111.60; ¹⁹F NMR (376 MHz, DMSO-d₆) δ−56.82; ESIMS m/z 321 ([M+H]⁺).

### Example 113: Preparation of methyl (4-(3-(4-amino-3-fluorophenyl)-1H-1,2,4-triazol-1-yl)phenyl)(trifluoromethyl)carbamate (C526)

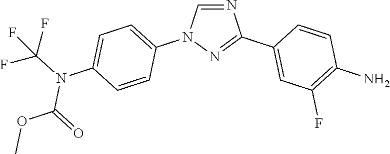 [SMILES: COC(=O)N(c1ccc(-n2cnc(-c3ccc(N)c(F)c3)n2)cc1)C(F)(F)F]

To an argon-degassed solution of methyl (4-(3-bromo-1H-1,2,4-triazol-1-yl)phenyl)(trifluoromethyl)carbamate (C502; 0.5 g, 1.36 mmol) in 1, 4-dioxane-water (2:1 ratio; 8 mL) were 2-fluoro-4-(4,4,5,5-tetramethyl-1,3,2-dioxaborolan-2-yl)aniline (0.35 g, 1.49 mmol) and sodium bicarbonate (0.34 g, 4.08 mmol). Tetrakis(triphenylphosphine)palladium(0) (0.08 g, 0.06 mmol) was added, and the reaction mixture was irradiated under microwave at 110° C. for 4 h. The reaction mixture was cooled to room temperature, poured into ice water (10 mL) and was extracted with EtOAc (3×5 mL). The organic layer was washed with water (5 mL) and brine (5 mL), dried over anhydrous Na₂SO₄, filtered, and concentrated under reduced pressure. Purification of the resultant material by column chromatography (silica gel 100-200 mesh) eluting with 60-80% EtOAc in petroleum ether afforded the title compound as an off-white solid (0.12 g, 28%): mp 159-163° C.; ¹H NMR (400 MHz, CDCl₃) δ 8.56 (s, 1H), 7.85-7.78 (m, 4H), 7.44-7.42 (m, 2H), 7.87-7.83 (m, 1H), 3.93 (br s, 2H), 3.81 (s, 3H); ¹⁹F NMR (376 MHz, CDCl₃) δ −54.64, −135.35; ESIMS m/z 396 ([M+H]⁺).

The following compound was prepared according to the procedure described in Example 113:

### Methyl (4-(3-(4-amino-3-methylphenyl)-1H-1,2,4-triazol-1-yl)phenyl)(trifluoromethyl)carbamate (C527)

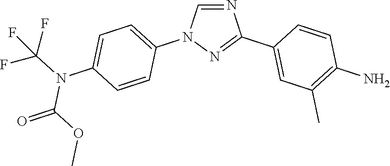 [SMILES: COC(=O)N(c1ccc(-n2cnc(-c3ccc(N)c(C)c3)n2)cc1)C(F)(F)F]

The title compound was prepared and was isolated as a pale brown solid (0.17 g, 40%): mp 178-182° C.; ¹H NMR (400 MHz, CDCl₃) δ 8.55 (s, 1H), 7.91-7.86 (m, 2H), 7.83-7.81 (m, 2H), 7.42-7.40 (m, 2H), 6.75 (d, J=8.0 Hz, 1H), 3.81 (s, 3H), 2.25 (s, 3H); ¹⁹F NMR (376 MHz, CDCl₃) δ −54.67; ESIMS m/z 392 ([M+H]⁺).

### Example 114: Preparation of (Z)-1-(3-(2-(1-methoxyethyl)-5-methylphenyl)-4-oxothiazolidin-2-ylidene)-3-(4-(1-(4-((trifluoromethyl)thio)phenyl)-1H-1,2,4-triazol-3-yl)phenyl)urea (J350)

4-(1-(4-((Trifluoromethyl)thio)phenyl)-1H-1,2,4-triazol-3-yl)aniline (C462; 50 mg, 0.149 mmol) was dissolved in DCM (0.743 mL, 0.2 M), and diisopropylethylamine (78 μL, 0.446 mmol) was added. After five minutes, bis(trichloromethyl) carbonate (17.6 mg, 0.059 mmol) was added, and the reaction mixture was allowed to stir for at room temperature for 40 minutes. 2-Imino-3-(2-(1-methoxyethyl)-5-methylphenyl)thiazolidin-4-one (prepared as in PCT International Application Publication WO 2017040194 A1; 39.3 mg, 0.149 mmol) was added. The reaction mixture was stirred at room temperature for 90 minutes, at which time LC-MS indicated reaction completion. Then, the reaction mixture was diluted with DCM (5 mL), shaken with water (5 mL) and passed through a phase separator, washing with DCM. The resulting filtrate was evaporated in vacuo, and the residue was dissolved in the minimal amount of EtOAc, to which heptanes (20 mL) was added, causing a solid to precipitate out of solution. The solid was collected by vacuum filtration, washed with hexanes, and was dried under high vacuum. The title compound was prepared and was isolated as a grey solid (60 mg, 62%).

The following molecules were prepared in accordance to the procedure in Example 114:

### (Z)-1-(2-Fluoro-4-(1-(4-((trifluoromethyl)sulfonyl)phenyl)-1H-1,2,4-triazol-3-yl)phenyl)-3-(3-(2-(1-methoxyethyl)-5-methylphenyl)-4-oxothiazolidin-2-ylidene)urea (J361)

The title compound was prepared and was isolated as a light orange solid (71 mg, 77%).

### (Z)-1-(3-(2-(1-Methoxyethyl)-5-methylphenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J362)

The title compound was prepared and was isolated as a light pink solid (66 mg, 71%).

### (Z)-1-(2-Fluoro-4-(1-(4-((trifluoromethyl)sulfonyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J363)

The title compound was prepared and was isolated as a light orange solid (63 mg, 65%).

### (Z)-1-(3-(5-(Dimethylamino)-2-isopropylphenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J364)

The title compound was prepared and was isolated as a light pink solid (66 mg, 72%).

### (Z)-1-(3-(5-Methyl-2-((2,2,2-trifluoroethoxy)methyl)phenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J365)

The title compound was prepared and was isolated as a tan solid (83 mg, 85%).

### (Z)-1-(3-(5-(Dimethylamino)-2-isopropylphenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J366)

The title compound was prepared and was isolated as an off-white solid (88 mg, 89%).

### (Z)-1-(3-(5-Methyl-2-((2,2,2-trifluoroethoxy)methyl)phenyl)-4-oxothiazolidin-2-ylidene)-3-(4-(1-(4-((trifluoromethyl)thio)phenyl)-1H-1,2,4-triazol-3-yl)phenyl)urea (J367)

The title compound was prepared and was isolated as a grey solid (97 mg, 86%).

### (Z)-1-(3-(5-(Dimethylamino)-2-isopropylphenyl)-4-oxothiazolidin-2-ylidene)-3-(4-(1-(4-((trifluoromethyl)thio)phenyl)-1H-1,2,4-triazol-3-yl)phenyl)urea (J368)

The title compound was prepared and was isolated as a light orange solid (59 mg, 58%).

### (Z)-1-(2-Fluoro-4-(1-(4-((trifluoromethyl)sulfonyl)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J369)

The title compound was prepared and was isolated as an off-white solid (67 mg, 68%).

### (Z)-1-(3-(5-(Dimethylamino)-2-propylphenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J370)

The title compound was prepared and was isolated as a dark yellow solid (79 mg, 84%).

### (Z)-1-(3-(5-Cyano-2-(3,3,3-trifluoropropoxy)phenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J372)

The title compound was prepared and was isolated as a purple solid (82 mg, 81%).

### (Z)-1-(3-(5-(Dimethylamino)-2-propylphenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J374)

The title compound was prepared and was isolated as a tan solid (64 mg, 69%).

### (Z)-1-(3-(5-Cyano-2-(3,3,3-trifluoropropoxy)phenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J376)

The title compound was prepared and was isolated as a light purple solid (97 mg, 95%).

### (Z)-1-(3-(5-(Dimethylamino)-2-isopropylphenyl)-4-oxothiazolidin-2-ylidene)-3-(2-fluoro-4-(1-(4-((trifluoromethyl)sulfinyl)phenyl)-1H-1,2,4-triazol-3-yl)phenyl)urea (J379)

The title compound was prepared and was isolated as an off-white solid (64 mg, 67%).

### (Z)-1-(2-Fluoro-4-(1-(4-((trifluoromethyl)sulfinyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J380)

The title compound was prepared and was isolated as a light pink solid (68 mg, 67%).

### (Z)-1-(2-Fluoro-4-(1-(4-((trifluoromethyl)sulfinyl)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J382)

The title compound was prepared and was isolated as a light pink solid (71 mg, 70%).

### (Z)-1-(3-(5-(Dimethylamino)-2-propylphenyl)-4-oxothiazolidin-2-ylidene)-3-(2-fluoro-4-(1-(4-((trifluoromethyl)sulfinyl)phenyl)-1H-1,2,4-triazol-3-yl)phenyl)urea (J383)

The title compound was prepared and was isolated as a light orange solid (71 mg, 74%).

### (Z)-1-(3-(5-(Dimethylamino)-4-fluoro-2-isopropylphenyl)-4-oxothiazolidin-2-ylidene)-3-(2-fluoro-4-(1-(4-((trifluoromethyl)sulfinyl)phenyl)-1H-1,2,4-triazol-3-yl)phenyl)urea (J384)

The title compound was prepared and was isolated as a beige solid (65 mg, 66%).

### (Z)-1-(3-(5-Cyano-2-(3,3,3-trifluoropropoxy)phenyl)-4-oxothiazolidin-2-ylidene)-3-(2-fluoro-4-(1-(4-((trifluoromethyl)sulfinyl)phenyl)-1H-1,2,4-triazol-3-yl)phenyl)urea (J385)

The title compound was prepared and was isolated as a light purple solid (84 mg, 81%).

### (Z)-1-(2-Fluoro-4-(1-(4-((trifluoromethyl)sulfinyl)phenyl)-1H-1,2,4-triazol-3-yl)phenyl)-3-(3-(5-methyl-2-(2,2,2-trifluoro-1-methoxyethyl)phenyl)-4-oxothiazolidin-2-ylidene)urea (J386)

The title compound was prepared and was isolated as a beige solid (72 mg, 71%).

### (Z)-1-(3-(5-Methyl-2-(3,3,3-trifluoropropoxy)phenyl)-4-oxothiazolidin-2-ylidene)-3-(4-(1-(4-((trifluoromethyl)thio)phenyl)-1H-1,2,4-triazol-3-yl)phenyl)urea (J387)

The title compound was prepared and was isolated as a beige solid (91 mg, 85%).

### (Z)-1-(3-(5-(Dimethylamino)-4-fluoro-2-isopropylphenyl)-4-oxothiazolidin-2-ylidene)-3-(4-(1-(4-((trifluoromethyl)thio)phenyl)-1H-1,2,4-triazol-3-yl)phenyl)urea (J389)

The title compound was prepared and was isolated as a light grey solid (69 mg, 67%).

### (Z)-1-(3-(5-Cyano-2-(3,3,3-trifluoropropoxy)phenyl)-4-oxothiazolidin-2-ylidene)-3-(4-(1-(4-((trifluoromethyl)thio)phenyl)-1H-1,2,4-triazol-3-yl)phenyl)urea (J392)

The title compound was prepared and was isolated as a purple solid (92 mg, 85%).

### (Z)-1-(3-(5-Methyl-2-((2,2,2-trifluoroethoxy)methyl)phenyl)-4-oxothiazolidin-2-ylidene)-3-(4-(3-(4-(trifluoromethoxy)phenyl)-1H-1,2,4-triazol-1-yl)phenyl)urea (J403)

The title compound was prepared and was isolated as a light orange solid (72 mg, 66%).

### (Z)-1-(3-(5-Methyl-2-(3,3,3-trifluoropropoxy)phenyl)-4-oxothiazolidin-2-ylidene)-3-(4-(3-(4-(trifluoromethoxy)phenyl)-1H-1,2,4-triazol-1-yl)phenyl)urea (J404)

The title compound was prepared and was isolated as an off-white solid (66 mg, 60%).

### (Z)-1-(3-(5-Methyl-2-(2,2,2-trifluoro-1-methoxyethyl)phenyl)-4-oxothiazolidin-2-ylidene)-3-(4-(3-(4-(trifluoromethoxy)phenyl)-1H-1,2,4-triazol-1-yl)phenyl)urea (J405)

The title compound was prepared and was isolated as a tan solid (73 mg, 67%).

### (Z)-1-(3-(5-Cyano-2-(3,3,3-trifluoropropoxy)phenyl)-4-oxothiazolidin-2-ylidene)-3-(4-(3-(4-(trifluoromethoxy)phenyl)-1H-1,2,4-triazol-1-yl)phenyl)urea (J406)

The title compound was prepared and was isolated as a light purple solid (43 mg, 38%).

### (Z)-1-(3-(5-Cyano-2-isopropylphenyl)-4-oxothiazolidin-2-ylidene)-3-(4-(3-(4-(trifluoromethoxy)phenyl)-1H-1,2,4-triazol-1-yl)phenyl)urea (J407)

The title compound was prepared and was isolated as a purple solid (69 mg, 69%).

### (Z)-1-(3-(5-(Dimethylamino)-2-isopropylphenyl)-4-oxothiazolidin-2-ylidene)-3-(4-(3-(4-(trifluoromethoxy)phenyl)-1H-1,2,4-triazol-1-yl)phenyl)urea (J408)

The title compound was prepared and was isolated as a light purple solid (53 mg, 51%).

### (Z)-1-(3-(5-(Dimethylamino)-2-propylphenyl)-4-oxothiazolidin-2-ylidene)-3-(4-(3-(4-(trifluoromethoxy)phenyl)-1H-1,2,4-triazol-1-yl)phenyl)urea (J409)

The title compound was prepared and was isolated as an off-white solid (40 mg, 32%).

### (Z)-1-(3-(5-(Dimethylamino)-4-fluoro-2-isopropylphenyl)-4-oxothiazolidin-2-ylidene)-3-(4-(3-(4-(trifluoromethoxy)phenyl)-1H-1,2,4-triazol-1-yl)phenyl)urea (J410)

The title compound was prepared and was isolated as an off-white solid (50 mg, 47%).

### (Z)-1-(2-Fluoro-4-(5-(4-(trifluoromethoxy)phenyl)-1,2,4-oxadiazol-3-yl)phenyl)-3-(3-(2-(1-methoxyethyl)-5-methylphenyl)-4-oxothiazolidin-2-ylidene)urea (J426)

The title compound was prepared and was isolated as a light pink solid (72 mg, 73%).

### (Z)-1-(2-Fluoro-4-(3-(4-(trifluoromethoxy)phenyl)-1,2,4-oxadiazol-5-yl)phenyl)-3-(3-(5-methyl-2-((2,2,2-trifluoroethoxy)methyl)phenyl)-4-oxothiazolidin-2-ylidene)urea (J427)

The title compound was prepared and was isolated as a tan solid (47 mg, 47%).

### (Z)-1-(2-Fluoro-4-(3-(4-(trifluoromethoxy)phenyl)-1,2,4-oxadiazol-5-yl)phenyl)-3-(3-(5-methyl-2-(2,2,2-trifluoro-1-methoxyethyl)phenyl)-4-oxothiazolidin-2-ylidene)urea (J428)

The title compound was prepared and was isolated as an orange solid (47 mg, 47%).

### (Z)-1-(2-Fluoro-4-(5-(4-(trifluoromethoxy)phenyl)-1,3,4-oxadiazol-2-yl)phenyl)-3-(3-(2-(1-methoxyethyl)-5-methylphenyl)-4-oxothiazolidin-2-ylidene)urea (J429)

The title compound was prepared and was isolated as a pink solid (42 mg, 44%).

### (Z)-1-(2-Fluoro-4-(5-(4-(trifluoromethoxy)phenyl)-1,3,4-oxadiazol-2-yl)phenyl)-3-(3-(5-methyl-2-((2,2,2-trifluoroethoxy)methyl)phenyl)-4-oxothiazolidin-2-ylidene)urea (J430)

The title compound was prepared and was isolated as a light orange solid (72 mg, 71%).

### (Z)-1-(3-(5-(Dimethylamino)-2-isopropylphenyl)-4-oxothiazolidin-2-ylidene)-3-(2-fluoro-4-(5-(4-(trifluoromethoxy)phenyl)-1,3,4-oxadiazol-2-yl)phenyl)urea (J431)

The title compound was prepared and was isolated as a pink solid (69 mg, 70%).

### (Z)-1-(3-(5-(Dimethylamino)-4-fluoro-2-isopropylphenyl)-4-oxothiazolidin-2-ylidene)-3-(2-fluoro-4-(5-(4-(trifluoromethoxy)phenyl)-1,3,4-oxadiazol-2-yl)phenyl)urea (J432)

The title compound was prepared and was isolated as a pink solid (53 mg, 54%).

### (Z)-1-(2-Fluoro-4-(5-(4-(trifluoromethoxy)phenyl)-1,3,4-oxadiazol-2-yl)phenyl)-3-(3-(5-methyl-2-(2,2,2-trifluoro-1-methoxyethyl)phenyl)-4-oxothiazolidin-2-ylidene)urea (J433)

The title compound was prepared and was isolated as a light orange solid (61 mg, 55%).

### (Z)-1-(2-Fluoro-4-(3-(4-(trifluoromethoxy)phenyl)-1,2,4-oxadiazol-5-yl)phenyl)-3-(3-(2-(1-methoxyethyl)-5-methylphenyl)-4-oxothiazolidin-2-ylidene)urea (J434)

The title compound was prepared and was isolated as a pale yellow solid (27 mg, 29%).

### (Z)-1-(3-(5-Methyl-2-(3,3,3-trifluoropropoxy)phenyl)-4-oxothiazolidin-2-ylidene)-3-(2-methyl-4-(3-(4-(trifluoromethoxy)phenyl)-1H-1,2,4-triazol-1-yl)phenyl)urea (J441)

The title compound was prepared and was isolated as an off-white solid (79 mg, 77%).

### (Z)-1-(3-(5-Methyl-2-((2,2,2-trifluoroethoxy)methyl)phenyl)-4-oxothiazolidin-2-ylidene)-3-(2-methyl-4-(3-(4-(trifluoromethoxy)phenyl)-1H-1,2,4-triazol-1-yl)phenyl)urea (J442)

The title compound was prepared and was isolated as a light orange solid (85 mg, 81%).

### (Z)-1-(3-(5-Methyl-2-(2,2,2-trifluoro-1-methoxyethyl)phenyl)-4-oxothiazolidin-2-ylidene)-3-(2-methyl-4-(3-(4-(trifluoromethoxy)phenyl)-1H-1,2,4-triazol-1-yl)phenyl)urea (J443)

The title compound was prepared and was isolated as a pink solid (90 mg, 84%).

### (Z)-1-(3-(5-(Dimethylamino)-2-(methoxymethyl)phenyl)-4-oxothiazolidin-2-ylidene)-3-(2-methyl-4-(3-(4-(trifluoromethoxy)phenyl)-1H-1,2,4-triazol-1-yl)phenyl)urea (J444)

The title compound was prepared and was isolated as a light orange solid (72 mg, 75%).

### (Z)-1-(2-Fluoro-4-(1-(5-(trifluoromethyl)pyridin-2-yl)-1H-imidazol-4-yl)phenyl)-3-(3-(2-(1-methoxyethyl)-5-methylphenyl)-4-oxothiazolidin-2-ylidene)urea (J461)

The title compound was prepared and was isolated as a dark red solid (37 mg, 44%).

### (Z)-1-(3-(5-(Dimethylamino)-2-(trifluoromethyl)phenyl)-4-oxothiazolidin-2-ylidene)-3-(2-methyl-4-(1-(4-(trifluoromethoxy)phenyl)-1H-imidazol-4-yl)phenyl)urea (J462)

The title compound was prepared and was isolated as a beige solid (11 mg, 11%).

### (Z)-1-(3-(2-(1-Methoxyethyl)-5-methylphenyl)-4-oxothiazolidin-2-ylidene)-3-(4-(1-(4-(trifluoromethoxy)phenyl)-1H-imidazol-4-yl)phenyl)urea (J463)

The title compound was prepared and was isolated as a beige solid (65 mg, 67%).

### (Z)-1-(2-Fluoro-4-(1-(4-(trifluoromethoxy)phenyl)-1H-imidazol-4-yl)phenyl)-3-(3-(2-isopropyl-5-methylphenyl)-4-oxothiazolidin-2-ylidene)urea (J473)

The title compound was prepared and was isolated as a tan solid (53 mg, 63%).

### (Z)-1-(3-(5-(Dimethylamino)-2-isopropylphenyl)-4-oxothiazolidin-2-ylidene)-3-(2-fluoro-4-(1-(4-(trifluoromethoxy)phenyl)-1H-imidazol-4-yl)phenyl)urea (J474)

The title compound was prepared and was isolated as a pink solid (69 mg, 65%).

### (Z)-1-(2-Fluoro-4-(1-(4-(trifluoromethoxy)phenyl)-1H-imidazol-4-yl)phenyl)-3-(3-(5-methyl-2-(3,3,3-trifluoropropoxy)phenyl)-4-oxothiazolidin-2-ylidene)urea (J476)

The title compound was prepared and was isolated as a pink solid (14.5 mg, 18%).

### (Z)-1-(3-(2-Isopropyl-5-methylphenyl)-4-oxothiazolidin-2-ylidene)-3-(2-methyl-4-(1-(4-(trifluoromethoxy)phenyl)-1H-imidazol-4-yl)phenyl)urea (J477)

The title compound was prepared and was isolated as an orange solid (40 mg, 43%).

### (Z)-1-(3-(5-Methyl-2-(2,2,2-trifluoro-1-methoxyethyl)phenyl)-4-oxothiazolidin-2-ylidene)-3-(2-methyl-4-(1-(4-(trifluoromethoxy)phenyl)-1H-imidazol-4-yl)phenyl)urea (J480)

The title compound was prepared and was isolated as an off-white solid (3.5 mg, 3%).

### (Z)-1-(3-(5-(Dimethylamino)-2-isopropylphenyl)-4-oxothiazolidin-2-ylidene)-3-(2-fluoro-4-(1-(5-(trifluoromethyl)pyridin-2-yl)-1H-imidazol-4-yl)phenyl)urea (J481)

The title compound was prepared and was isolated as a light orange solid (24 mg, 31%).

### (Z)-1-(2-Fluoro-4-(1-(5-(trifluoromethyl)pyridin-2-yl)-1H-imidazol-4-yl)phenyl)-3-(3-(2-isopropyl-5-methylphenyl)-4-oxothiazolidin-2-ylidene)urea (J482)

The title compound was prepared and was isolated as a beige solid (41 mg, 54%).

### (Z)-1-(3-(5-(Dimethylamino)-2-(trifluoromethyl)phenyl)-4-oxothiazolidin-2-ylidene)-3-(2-fluoro-4-(1-(5-(trifluoromethyl)pyridin-2-yl)-1H-imidazol-4-yl)phenyl)urea (J483)

The title compound was prepared and was isolated as a beige solid (53 mg, 64%).

### (Z)-1-(2-Fluoro-4-(1-(5-(trifluoromethyl)pyridin-2-yl)-1H-imidazol-4-yl)phenyl)-3-(3-(5-methyl-2-(3,3,3-trifluoropropoxy)phenyl)-4-oxothiazolidin-2-ylidene)urea (J484)

The title compound was prepared and was isolated as an off-white solid (55 mg, 66%).

### (Z)-1-(2-Fluoro-4-(1-(5-(trifluoromethyl)pyridin-2-yl)-1H-imidazol-4-yl)phenyl)-3-(3-(5-methyl-2-(2,2,2-trifluoro-1-methoxyethyl)phenyl)-4-oxothiazolidin-2-ylidene)urea (J485)

The title compound was prepared and was isolated as a tan solid (47 mg, 56%).

### (Z)-1-(3-(2-(1-Ethoxyethyl)-5-methylphenyl)-4-oxothiazolidin-2-ylidene)-3-(2-methyl-4-(1-(4-(trifluoromethoxy)phenyl)-1H-imidazol-4-yl)phenyl)urea (J486)

The title compound was prepared and was isolated as a beige solid (50 mg, 50%).

### (Z)-1-(2-Fluoro-4-(1-(5-(trifluoromethyl)pyridin-2-yl)-1H-imidazol-4-yl)phenyl)-3-(3-(5-methyl-2-((2,2,2-trifluoroethoxy)methyl)phenyl)-4-oxothiazolidin-2-ylidene)urea (J487)

The title compound was prepared and was isolated as a pink solid (15 mg, 18%).

### (Z)-1-(2-Fluoro-4-(5-(4-(trifluoromethoxy)phenyl)-1,3,4-thiadiazol-2-yl)phenyl)-3-(3-(2-isopropyl-5-methylphenyl)-4-oxothiazolidin-2-ylidene)urea (J499)

The title compound was prepared and was isolated as a dark orange solid (49 mg, 52%).

### (Z)-1-(2-Fluoro-4-(5-(4-(trifluoromethoxy)phenyl)-1,3,4-thiadiazol-2-yl)phenyl)-3-(3-(5-methyl-2-((2,2,2-trifluoroethoxy)methyl)phenyl)-4-oxothiazolidin-2-ylidene)urea (J500)

The title compound was prepared and was isolated as a dark orange solid (68 mg, 65%).

### (Z)-1-(2-Fluoro-4-(5-(4-(trifluoromethoxy)phenyl)-1,3,4-thiadiazol-2-yl)phenyl)-3-(3-(5-methyl-2-(3,3,3-trifluoropropoxy)phenyl)-4-oxothiazolidin-2-ylidene)urea (J501)

The title compound was prepared and was isolated as a tan solid (73 mg, 70%).

### (Z)-1-(2-Fluoro-4-(5-(4-(trifluoromethoxy)phenyl)-1,3,4-thiadiazol-2-yl)phenyl)-3-(3-(5-methyl-2-(4,4,4-trifluorobutoxy)phenyl)-4-oxothiazolidin-2-ylidene)urea (J502)

The title compound was prepared and was isolated as a light orange solid (60 mg, 57%).

### (Z)-1-(3-(2-(Ethoxymethyl)-5-methylphenyl)-4-oxothiazolidin-2-ylidene)-3-(2-fluoro-4-(5-(4-(trifluoromethoxy)phenyl)-1,3,4-thiadiazol-2-yl)phenyl)urea (J503)

The title compound was prepared and was isolated as an orange solid (59 mg, 62%).

### (Z)-1-(2-Fluoro-4-(5-(4-(trifluoromethoxy)phenyl)-1,3,4-thiadiazol-2-yl)phenyl)-3-(3-(2-(1-methoxyethyl)-5-methylphenyl)-4-oxothiazolidin-2-ylidene)urea (J504)

The title compound was prepared and was isolated as a light orange solid (63 mg, 65%).

### (Z)-1-(2-Fluoro-4-(5-(4-(trifluoromethoxy)phenyl)-1,3,4-thiadiazol-2-yl)phenyl)-3-(3-(5-methyl-2-(2,2,2-trifluoro-1-methoxyethyl)phenyl)-4-oxothiazolidin-2-ylidene)urea (J505)

The title compound was prepared and was isolated as a dark yellow solid (57 mg, 55%).

### (Z)-1-(3-(5-(Dimethylamino)-2-isopropylphenyl)-4-oxothiazolidin-2-ylidene)-3-(2-fluoro-4-(5-(4-(trifluoromethoxy)phenyl)-1,3,4-thiadiazol-2-yl)phenyl)urea (J506)

The title compound was prepared and was isolated as a pink solid (72 mg, 74%).

### (Z)-1-(3-(5-(Dimethylamino)-4-fluoro-2-isopropylphenyl)-4-oxothiazolidin-2-ylidene)-3-(2-fluoro-4-(5-(4-(trifluoromethoxy)phenyl)-1,3,4-thiadiazol-2-yl)phenyl)urea (J507)

The title compound was prepared and was isolated as a pink solid (57 mg, 57%).

### (Z)-1-(3-(2-(1-Methoxyethyl)-5-methylphenyl)-4-oxothiazolidin-2-ylidene)-3-(2-methyl-4-(1-(5-(trifluoromethyl)pyridin-2-yl)-1H-imidazol-4-yl)phenyl)urea (J508)

The title compound was prepared and was isolated as a beige solid (82 mg, 85%).

### (Z)-1-(3-(2-Isopropyl-5-methylphenyl)-4-oxothiazolidin-2-ylidene)-3-(2-methyl-4-(1-(5-(trifluoromethyl)pyridin-2-yl)-1H-imidazol-4-yl)phenyl)urea (J509)

The title compound was prepared and was isolated as a beige solid (74 mg, 79%).

### (Z)-1-(3-(5-(Dimethylamino)-2-isopropylphenyl)-4-oxothiazolidin-2-ylidene)-3-(2-methyl-4-(1-(5-(trifluoromethyl)pyridin-2-yl)-1H-imidazol-4-yl)phenyl)urea (J510)

The title compound was prepared and was isolated as a tan solid (82 mg, 83%).

### (Z)-1-(3-(5-Methyl-2-((2,2,2-trifluoroethoxy)methyl)phenyl)-4-oxothiazolidin-2-ylidene)-3-(2-methyl-4-(1-(5-(trifluoromethyl)pyridin-2-yl)-1H-imidazol-4-yl)phenyl)urea (J511)

The title compound was prepared and was isolated as an orange solid (91 mg, 86%).

### (Z)-1-(3-(5-Methyl-2-(3,3,3-trifluoropropoxy)phenyl)-4-oxothiazolidin-2-ylidene)-3-(2-methyl-4-(1-(5-(trifluoromethyl)pyridin-2-yl)-1H-imidazol-4-yl)phenyl)urea (J512)

The title compound was prepared and was isolated as a light orange solid (100 mg, 95%).

### (Z)-1-(3-(2-(1-Methoxyethyl)-5-methylphenyl)-4-oxothiazolidin-2-ylidene)-3-(4-(1-(4-(trifluoromethyl)phenyl)-1H-imidazol-4-yl)phenyl)urea (J513)

The title compound was prepared and was isolated as a pale yellow solid (38.6 mg, 65%).

### (Z)-1-(3-(2-Isopropyl-5-methylphenyl)-4-oxothiazolidin-2-ylidene)-3-(4-(1-(4-(trifluoromethyl)phenyl)-1H-imidazol-4-yl)phenyl)urea (J514)

The title compound was prepared and was isolated as a light orange solid (44 mg, 76%).

### (Z)-1-(3-(5-(Dimethylamino)-2-isopropylphenyl)-4-oxothiazolidin-2-ylidene)-3-(4-(1-(4-(trifluoromethyl)phenyl)-1H-imidazol-4-yl)phenyl)urea (J515)

The title compound was prepared and was isolated as an off-white solid (42 mg, 69%).

### (Z)-1-(3-(5-Methyl-2-((2,2,2-trifluoroethoxy)methyl)phenyl)-4-oxothiazolidin-2-ylidene)-3-(4-(1-(4-(trifluoromethyl)phenyl)-1H-imidazol-4-yl)phenyl)urea (J516)

The title compound was prepared and was isolated as a pink solid (39 mg, 60%).

### (Z)-1-(3-(5-Methyl-2-(3,3,3-trifluoropropoxy)phenyl)-4-oxothiazolidin-2-ylidene)-3-(4-(1-(4-(trifluoromethyl)phenyl)-1H-imidazol-4-yl)phenyl)urea (J517)

The title compound was prepared and was isolated as a beige solid (44 mg, 68%).

### (Z)-1-(3-(2-(1-Methoxyethyl)-5-methylphenyl)-4-oxothiazolidin-2-ylidene)-3-(4-(1-(5-(trifluoromethyl)pyridin-2-yl)-1H-imidazol-4-yl)phenyl)urea (J518)

The title compound was prepared and was isolated as an off-white solid (67 mg, 68%).

### (Z)-1-(3-(2-Isopropyl-5-methylphenyl)-4-oxothiazolidin-2-ylidene)-3-(4-(1-(5-(trifluoromethyl)pyridin-2-yl)-1H-imidazol-4-yl)phenyl)urea (J519)

The title compound was prepared and was isolated as a pink solid (54 mg, 56%).

### (Z)-1-(3-(5-(Dimethylamino)-2-isopropylphenyl)-4-oxothiazolidin-2-ylidene)-3-(4-(1-(5-(trifluoromethyl)pyridin-2-yl)-1H-imidazol-4-yl)phenyl)urea (J520)

The title compound was prepared and was isolated as a light orange solid (46 mg, 46%).

### (Z)-1-(3-(5-Methyl-2-((2,2,2-trifluoroethoxy)methyl)phenyl)-4-oxothiazolidin-2-ylidene)-3-(4-(1-(5-(trifluoromethyl)pyridin-2-yl)-1H-imidazol-4-yl)phenyl)urea (J521)

The title compound was prepared and was isolated as a light purple solid (54 mg, 50%).

### (Z)-1-(3-(5-Methyl-2-(3,3,3-trifluoropropoxy)phenyl)-4-oxothiazolidin-2-ylidene)-3-(4-(1-(5-(trifluoromethyl)pyridin-2-yl)-1H-imidazol-4-yl)phenyl)urea (J522)

The title compound was prepared and was isolated as an off-white solid (99 mg, 92%).

### (Z)-1-(3-(2-Isopropyl-5-methylphenyl)-4-oxothiazolidin-2-ylidene)-3-(2-methyl-4-(1-(4-(trifluoromethyl)phenyl)-1H-imidazol-4-yl)phenyl)urea (J523)

The title compound was prepared and was isolated as a light orange solid (50.6 mg, 67%).

### (Z)-1-(3-(5-(Dimethylamino)-2-isopropylphenyl)-4-oxothiazolidin-2-ylidene)-3-(2-methyl-4-(1-(4-(trifluoromethyl)phenyl)-1H-imidazol-4-yl)phenyl)urea (J524)

The title compound was prepared and was isolated as a yellow solid (48.5 mg, 61%).

### (Z)-1-(3-(2-(1-Methoxyethyl)-5-methylphenyl)-4-oxothiazolidin-2-ylidene)-3-(2-methyl-4-(1-(4-(trifluoromethyl)phenyl)-1H-imidazol-4-yl)phenyl)urea (J525)

The title compound was prepared and was isolated as a yellow solid (43 mg, 56%).

### (Z)-1-(3-(5-Methyl-2-((2,2,2-trifluoroethoxy)methyl)phenyl)-4-oxothiazolidin-2-ylidene)-3-(2-methyl-4-(1-(4-(trifluoromethyl)phenyl)-1H-imidazol-4-yl)phenyl)urea (J526)

The title compound was prepared and was isolated as a tan solid (30 mg, 36%).

### (Z)-1-(3-(5-Methyl-2-(3,3,3-trifluoropropoxy)phenyl)-4-oxothiazolidin-2-ylidene)-3-(2-methyl-4-(1-(4-(trifluoromethyl)phenyl)-1H-imidazol-4-yl)phenyl)urea (J527)

The title compound was prepared and was isolated as a yellow solid (51 mg, 61%).

### (Z)-1-(3-(5-(Dimethylamino)-2-isopropylphenyl)-4-oxothiazolidin-2-ylidene)-3-(4-(1-(4-(trifluoromethoxy)phenyl)-1H-imidazol-4-yl)phenyl)urea (J529)

The title compound was prepared and was isolated as a beige solid (56 mg, 57%).

### (Z)-1-(2-Fluoro-4-(1-(4-(trifluoromethyl)phenyl)-1H-imidazol-4-yl)phenyl)-3-(3-(5-methyl-2-(3,3,3-trifluoropropoxy)phenyl)-4-oxothiazolidin-2-ylidene)urea (J532)

The title compound was prepared and was isolated as a gray solid (12.2 mg, 15%).

### (Z)-1-(3-(5-(Dimethylamino)-2-(methoxymethyl)phenyl)-4-oxothiazolidin-2-ylidene)-3-(2-methyl-4-(1-(4-(trifluoromethoxy)phenyl)-1H-imidazol-4-yl)phenyl)urea (J533)

The title compound was prepared and was isolated as a pink solid (12 mg, 12%).

### Example 115: Preparation of (Z)-1-(3-(5-(dimethylamino)-4-fluoro-2-isopropylphenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J371)

2-Fluoro-4-(1-(4-((trifluoromethyl)sulfonyl)phenyl)-1H-1,2,4-triazol-3-yl)aniline (C463; 50 mg, 0.129 mmol) was dissolved in DCM (647 μL, 0.2 M), and diisopropylethylamine (67.8 μL, 0.388 mmol) was added. After five minutes, bis(trichloromethyl) carbonate (15.4 mg, 0.052 mmol) was added, and the reaction mixture was allowed to stir at room temperature for 40 minutes. 3-(5-(Dimethylamino)-4-fluoro-2-isopropylphenyl)-2-iminothiazolidin-4-one (C103; 38.2 mg, 0.129 mmol) was added. The reaction mixture was stirred at room temperature for 90 minutes, at which time LC-MS indicated reaction completion. The reaction mixture was diluted with DCM (5 mL), was shaken with water (5 mL), and was passed through a phase separator, washing with DCM. The resulting filtrate was evaporated in vacuo, and the residue was dissolved in the minimal amount of EtOAc and precipitated with heptanes. The resulting solids were collected by vacuum filtration, washing with hexanes. The collected solids were dissolved in DCM (10 mL) and washed twice with saturated aqueous sodium bicarbonate and twice with brine. The organic layer was passed through a phase separator and concentrated in vacuo. The title compound was isolated as an off-white solid (80 mg, 85%).

The following molecules were prepared in accordance to the procedure in Example 115:

### (Z)-1-(3-(5-Methyl-2-(3,3,3-trifluoropropoxy)phenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J373)

The title compound was prepared and was isolated as a tan solid (90 mg, 90%).

### (Z)-1-(3-(5-(Dimethylamino)-4-fluoro-2-isopropylphenyl)-4-oxothiazolidin-2-ylidene)-3-(2-methyl-4-(1-(4-((trifluoromethyl)sulfonyl)phenyl)-1H-1,2,4-triazol-3-yl)phenyl)urea (J375)

The title compound was prepared and was isolated as a bright yellow solid (84 mg, 88%).

### (Z)-1-(3-(5-(Dimethylamino)-2-propylphenyl)-4-oxothiazolidin-2-ylidene)-3-(4-(1-(4-((trifluoromethyl)thio)phenyl)-1H-1,2,4-triazol-3-yl)phenyl)urea (J388)

The title compound was prepared and was isolated as a light brown solid (89 mg, 89%).

### Example 116: Preparation of (Z)-1-(3-(5-(dimethylamino)-2-isopropylphenyl)-4-oxothiazolidin-2-ylidene)-3-(2-fluoro-4-(3-(4-(trifluoromethoxy)phenyl)-1,2,4-oxadiazol-5-yl)phenyl)urea (J435)

2-Fluoro-4-(3-(4-(trifluoromethoxy)phenyl)-1,2,4-oxadiazol-5-yl)aniline (C494; 50 mg, 0.147 mmol) was dissolved in DCM (0.737 mL, 0.2 M), and diisopropylethylamine (57.1 mg, 0.442 mmol) was added. After five minutes, bis(trichloromethyl) carbonate (17.5 mg, 0.059 mmol) was added. The reaction mixture was allowed to stir at room temperature for 40 minutes. 3-(5-(Dimethylamino)-2-isopropylphenyl)-2-iminothiazolidin-4-one (C126; 40.9 mg, 0.147 mmol) was added. The reaction mixture was allowed to stir at room temperature overnight. After 16 h, LC-MS indicated reaction completion. The reaction was diluted with DCM (5 mL), was shaken with water (5 mL), and was passed through a phase separator. The filtrate was evaporated in vacuo, and the residue was dissolved in a minimal amount of EtOAc and precipitated with heptanes. The solids were collected by vacuum filtration, washed with hexanes, and dried over high vacuum overnight. Purification of the impure residue by normal-phase high-performance liquid chromatography eluting with 0-40% acetone-hexanes yielded a tan solid (21 mg, 21%).

The following molecules were prepared in accordance to the procedure in Example 116:

### (Z)-1-(3-(5-Methyl-2-(4,4,4-trifluorobutoxy)phenyl)-4-oxothiazolidin-2-ylidene)-3-(2-methyl-4-(3-(4-(trifluoromethoxy)phenyl)-1H-1,2,4-triazol-1-yl)phenyl)urea (J465)

The title compound was prepared and was isolated as an off-white solid (36 mg, 33%).

### (Z)-1-(3-(2-Isopropyl-5-methylphenyl)-4-oxothiazolidin-2-ylidene)-3-(2-methyl-4-(3-(4-(trifluoromethoxy)phenyl)-1H-1,2,4-triazol-1-yl)phenyl)urea (J466)

The title compound was prepared and was isolated as a tan solid (25 mg, 27%).

### (Z)-1-(3-(2-(1-Methoxyethyl)-5-methylphenyl)-4-oxothiazolidin-2-ylidene)-3-(2-methyl-4-(3-(4-(trifluoromethoxy)phenyl)-1H-1,2,4-triazol-1-yl)phenyl)urea (J467)

The title compound was prepared and was isolated as a white solid (23 mg, 24%).

### (Z)-1-(3-(5-(Dimethylamino)-2-(trifluoromethyl)phenyl)-4-oxothiazolidin-2-ylidene)-3-(2-methyl-4-(3-(4-(trifluoromethoxy)phenyl)-1H-1,2,4-triazol-1-yl)phenyl)urea (J468)

The title compound was prepared and was isolated as a tan solid (22 mg, 21%).

### (Z)-1-(3-(5-(Dimethylamino)-2-isopropylphenyl)-4-oxothiazolidin-2-ylidene)-3-(2-methyl-4-(3-(4-(trifluoromethoxy)phenyl)-1H-1,2,4-triazol-1-yl)phenyl)urea (J469)

The title compound was prepared and was isolated as an off-white solid (28 mg, 28%).

### (Z)-1-(3-(5-(Dimethylamino)-4-fluoro-2-isopropylphenyl)-4-oxothiazolidin-2-ylidene)-3-(2-methyl-4-(3-(4-(trifluoromethoxy)phenyl)-1H-1,2,4-triazol-1-yl)phenyl)urea (J470)

The title compound was prepared and was isolated as a white solid (11 mg, 11%).

### (Z)-1-(3-(2-(Ethoxymethyl)-5-methylphenyl)-4-oxothiazolidin-2-ylidene)-3-(2-methyl-4-(3-(4-(trifluoromethoxy)phenyl)-1H-1,2,4-triazol-1-yl)phenyl)urea (J471)

The title compound was prepared and was isolated as a tan solid (2.1 mg, 2%).

### (Z)-1-(3-(2-(1-Ethoxyethyl)-5-methylphenyl)-4-oxothiazolidin-2-ylidene)-3-(2-methyl-4-(3-(4-(trifluoromethoxy)phenyl)-1H-1,2,4-triazol-1-yl)phenyl)urea (J472)

The title compound was prepared and was isolated as an off-white solid (20 mg, 20%).

### (Z)-1-(2-Fluoro-4-(1-(4-((trifluoromethyl)sulfinyl)phenyl)-1H-1,2,4-triazol-3-yl)phenyl)-3-(3-(2-(1-methoxyethyl)-5-methylphenyl)-4-oxothiazolidin-2-ylidene)urea (J536)

The title compound was prepared and was isolated as a white solid (25 mg, 27%).

### Example 117: Preparation of (Z)-1-(2-cyano-4-(1-(4-((trifluoromethyl)sulfonyl)phenyl)-1H-1,2,4-triazol-3-yl)phenyl)-3-(3-(5-methyl-2-(3,3,3-trifluoropropoxy)phenyl)-4-oxothiazolidin-2-ylidene)urea (J402)

2-Amino-5-(1-(4-((trifluoromethyl)sulfonyl)phenyl)-1H-1,2,4-triazol-3-yl)benzonitrile (C465; 50 mg, 0.127 mmol) was dissolved in THF (1.0 mL), and 2-imino-3-(5-methyl-2-(3,3,3-trifluoropropoxy)phenyl)thiazolidin-4-one (C99; 40.5 mg, 0.127 mmol) was added with acetonitrile (1.0 mL). Diisopropylethylamine (0.067 mL, 0.381 mmol) was added, the white precipitate began to dissolve. The reaction mixture was allowed to stir at room temperature overnight. LC-MS indicated reaction completion. The solvent was removed in vacuo. The residue was dissolved in DCM (5 mL), and the mixture shaken with water (5 mL) and passed through a phase separator, washing with DCM. The filtrate was concentrated in vacuo, and the residue was dissolved with a minimal amount of EtOAc. Heptanes were added, causing a solid to precipitate. The solid was collected by vacuum filtration, washed with hexanes, and dried under high vacuum to afford the title compound as a tan solid (48 mg, 46%), which required no further purification.

### Example 118: Preparation of (Z)-1-(2-cyano-4-(1-(4-((trifluoromethyl)sulfonyl)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J411)

THF (1 mL) was used to solubilize 2-amino-5-(1-(4-((trifluoromethyl)sulfonyl)phenyl)-1H-1,2,4-triazol-3-yl)benzonitrile (C465; 50 mg, 0.127 mmol) and 4-nitrophenyl carbonochloridate (25.6 mg, 0.127 mmol). After 20 minutes the mixture became a white, opaque slurry. The solvent was removed in vacuo, and 2-imino-3-(5-methyl-2-((2,2,2-trifluoroethoxy)methyl)phenyl)thiazolidin-4-one (C117; 40.5 mg, 0.127 mmol) was added with acetonitrile (1.0 mL). Diisopropylethylamine (0.067 mL, 0.381 mmol) was added, the white precipitate began to dissolve. The reaction mixture was allowed to stir at room temperature overnight. LC-MS indicated reaction completion. The solvent was removed in vacuo, and the residue was dissolved in DCM (5 mL) and shaken with water (5 mL). The mixture was passed through a phase separator, washing with more DCM and water. The filtrate was concentrated in vacuo. The residue was dissolved with a minimal amount of EtOAc, and heptanes was added, causing a solid to precipitate. The solid was collected by vacuum filtration and washed with hexanes. Purification of the solid by normal-phase high-performance liquid chromatography eluting with 0-40% acetone-hexanes yielded the title compound as a pale yellow solid (21 mg, 20%).

The following molecules were prepared in accordance to the procedure in Example 118:

### (Z)-1-(3-(5-Cyano-2-(3,3,3-trifluoropropoxy)phenyl)-4-oxothiazolidin-2-ylidene)-3-(2-cyano-4-(1-(4-((trifluoromethyl)sulfonyl)phenyl)-1H-1,2,4-triazol-3-yl)phenyl)urea (J412)

The title compound was prepared and was isolated as a tan solid (10 mg, 10%).

### (Z)-1-(3-(5-Cyano-2-isopropylphenyl)-4-oxothiazolidin-2-ylidene)-3-(2-cyano-4-(1-(4-((trifluoromethyl)sulfonyl)phenyl)-1H-1,2,4-triazol-3-yl)phenyl)urea (J413)

The title compound was prepared and was isolated as a beige solid (34 mg, 37%).

### (Z)-1-(2-Cyano-4-(1-(4-((trifluoromethyl)sulfonyl)phenyl)-1H-1,2,4-triazol-3-yl)phenyl)-3-(3-(5-(dimethylamino)-2-isopropylphenyl)-4-oxothiazolidin-2-ylidene)urea (J414)

The title compound was prepared and was isolated as a tan solid (438 mg, 46%).

### (Z)-1-(3-(5-Methyl-2-((2,2,2-trifluoroethoxy)methyl)phenyl)-4-oxothiazolidin-2-ylidene)-3-(4-(1-(4-(trifluoromethoxy)phenyl)-1H-1,2,3-triazol-4-yl)phenyl)urea (J490)

The title compound was prepared and was isolated as a tan solid (8.0 mg, 7%).

### (Z)-1-(3-(5-Methyl-2-(3,3,3-trifluoropropoxy)phenyl)-4-oxothiazolidin-2-ylidene)-3-(4-(1-(4-(trifluoromethoxy)phenyl)-1H-1,2,3-triazol-4-yl)phenyl)urea (J491)

The title compound was prepared and was isolated as an off-white solid (41 mg, 39%).

### (Z)-1-(3-(5-Methyl-2-(4,4,4-trifluorobutoxy)phenyl)-4-oxothiazolidin-2-ylidene)-3-(4-(1-(4-(trifluoromethoxy)phenyl)-1H-1,2,3-triazol-4-yl)phenyl)urea (J492)

The title compound was prepared and was isolated as an off-white solid (40 mg, 37%).

### (Z)-1-(3-(2-(Ethoxymethyl)-5-methylphenyl)-4-oxothiazolidin-2-ylidene)-3-(4-(1-(4-(trifluoromethoxy)phenyl)-1H-1,2,3-triazol-4-yl)phenyl)urea (J493)

The title compound was prepared and was isolated as a light orange solid (47 mg, 45%).

### (Z)-1-(3-(5-Methyl-2-(2,2,2-trifluoro-1-methoxyethyl)phenyl)-4-oxothiazolidin-2-ylidene)-3-(4-(1-(4-(trifluoromethoxy)phenyl)-1H-1,2,3-triazol-4-yl)phenyl)urea (J494)

The title compound was prepared and was isolated as a tan solid (34 mg, 29%).

### (Z)-1-(3-(5-(Dimethylamino)-2-(trifluoromethyl)phenyl)-4-oxothiazolidin-2-ylidene)-3-(4-(1-(4-(trifluoromethoxy)phenyl)-1H-1,2,3-triazol-4-yl)phenyl)urea (J495)

The title compound was prepared and was isolated as a pink solid (30.5 mg, 30%).

### (Z)-1-(3-(5-(Dimethylamino)-2-(methoxymethyl)phenyl)-4-oxothiazolidin-2-ylidene)-3-(4-(1-(4-(trifluoromethoxy)phenyl)-1H-1,2,3-triazol-4-yl)phenyl)urea (J496)

The title compound was prepared and was isolated as a light orange solid (38 mg, 39%).

### Example 119: Preparation of (Z)-1-(2-fluoro-4-(1-(4-(pentafluoro-λ⁶-sulfaneyl)phenyl)-1H-imidazol-4-yl)phenyl)-3-(3-(2-isopropyl-5-methylphenyl)-4-oxothiazolidin-2-ylidene)urea (J589)

2-Fluoro-4-(1-(4-pentafluoro-λ⁶-sulfaneyl)phenyl)-1H-imidazol-4-yl)aniline (C467; 50 mg, 0.132 mmol) was suspended in THF (1 mL), and to the resulting suspension was added 4-nitrophenyl carbonochloridate (26.6 mg, 0.132 mmol). After sitting at room temperature for 1 h, 2-imino-3-(2-isopropyl-5-methylphenyl)thiazolidin-4-one (69.1 μL, 0.395 mmol) was added. The reaction mixture was allowed to proceed at room temperature overnight. After stirring overnight, LC-MS indicated reaction completion. The solvent was removed in vacuo. Purification of the residue by normal-phase high-performance liquid chromatography eluting with 0-40% acetone-hexanes yielded the title compound as a tan solid (56 mg, 59%).

The following molecules were prepared in accordance to the procedure in Example 119:

### (Z)-1-(2-Fluoro-4-(1-(4-(pentafluoro-λ⁶-sulfaneyl)phenyl)-1H-imidazol-4-yl)phenyl)-3-(3-(5-methyl-2-propylphenyl)-4-oxothiazolidin-2-ylidene)urea (J590)

The title compound was prepared and was isolated as a tan solid (68 mg, 71%).

### (Z)-1-(2-Fluoro-4-(1-(4-(pentafluoro-λ⁶-sulfaneyl)phenyl)-1H-imidazol-4-yl)phenyl)-3-(3-(2-(1-methoxyethyl)-5-methylphenyl)-4-oxothiazolidin-2-ylidene)urea (J591)

The title compound was prepared and was isolated as a beige solid (65 mg, 67%).

### (Z)-1-(2-Fluoro-4-(1-(4-(pentafluoro-λ⁶-sulfaneyl)phenyl)-1H-imidazol-4-yl)phenyl)-3-(3-(2-(methoxymethyl)-5-methylphenyl)-4-oxothiazolidin-2-ylidene)urea (J592)

The title compound was prepared and was isolated as a light orange solid (43 mg, 47%).

### (Z)-1-(3-(2-(Ethoxymethyl)-5-methylphenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J593)

The title compound was prepared and was isolated as a tan solid (24 mg, 25%).

### (Z)-1-(3-(2-(1-Ethoxyethyl)-5-methylphenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J594)

The title compound was prepared and was isolated as a yellow solid (53 mg, 53%).

### (Z)-1-(2-Fluoro-4-(1-(4-(pentafluoro-λ⁶-sulfaneyl)phenyl)-1H-imidazol-4-yl)phenyl)-3-(3-(5-methyl-2-propoxyphenyl)-4-oxothiazolidin-2-ylidene)urea (J595)

The title compound was prepared and was isolated as a light orange solid (65 mg, 67%).

### (Z)-1-(2-Fluoro-4-(1-(4-(pentafluoro-λ⁶-sulfaneyl)phenyl)-1H-imidazol-4-yl)phenyl)-3-(3-(5-methyl-2-(2,2,2-trifluoroethoxy)phenyl)-4-oxothiazolidin-2-ylidene)urea (J596)

The title compound was prepared and was isolated as a tan solid (69 mg, 70%).

### (Z)-1-(2-Fluoro-4-(1-(4-(pentafluoro-λ⁶-sulfaneyl)phenyl)-1H-imidazol-4-yl)phenyl)-3-(3-(5-methyl-2-(3,3,3-trifluoropropoxy)phenyl)-4-oxothiazolidin-2-ylidene)urea (J597)

The title compound was prepared and was isolated as a yellow solid (57 mg, 56%).

### (Z)-1-(2-Fluoro-4-(1-(4-(pentafluoro-λ⁶-sulfaneyl)phenyl)-1H-imidazol-4-yl)phenyl)-3-(3-(5-methyl-2-((2,2,2-trifluoroethoxy)methyl)phenyl)-4-oxothiazolidin-2-ylidene)urea (J598)

The title compound was prepared and was isolated as a tan solid (65 mg, 65%).

### (Z)-1-(2-Fluoro-4-(1-(4-(pentafluoro-λ⁶-sulfaneyl)phenyl)-1H-imidazol-4-yl)phenyl)-3-(3-(5-methyl-2-(2,2,2-trifluoro-1-methoxyethyl)phenyl)-4-oxothiazolidin-2-ylidene)urea (J599)

The title compound was prepared and was isolated as a beige solid (89 mg, 84%).

### (Z)-1-(3-(5-(Dimethylamino)-2-(trifluoromethyl)phenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J600)

The title compound was prepared and was isolated as a light orange solid (77 mg, 80%).

### (Z)-1-(3-(5-(Dimethylamino)-2-(methoxymethyl)phenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J601)

The title compound was prepared and was isolated as a light orange solid (68 mg, 72%).

### (Z)-1-(3-(5-(Dimethylamino)-2-propylphenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J602)

The title compound was prepared and was isolated as a light orange solid (89 mg, 96%).

### (Z)-1-(3-(5-(Dimethylamino)-2-isopropylphenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J603)

The title compound was prepared and was isolated as a light orange solid (66 mg, 70%).

### (Z)-1-(3-(5-(Dimethylamino)-4-fluoro-2-isopropylphenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J604)

The title compound was prepared and was isolated as a light orange solid (64 mg, 63%).

### (Z)-1-(3-(5-Cyano-2-isopropylphenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J605)

The title compound was prepared and was isolated as a brown solid (50 mg, 64%).

### (Z)-1-(3-(5-Cyano-2-(1-methoxyethyl)phenyl)-4-oxothiazolidin-2-ylidene)-3-(2-fluoro-4-(1-(4-(pentafluoro-λ⁶-sulfaneyl)phenyl)-1H-imidazol-4-yl)phenyl)urea (J606)

The title compound was prepared and was isolated as a brown solid (47 mg, 56%).

### Example 120: Preparation of (Z)-1-(3-(2-isopropyl-5-methylphenyl)-4-oxothiazolidin-2-ylidene)-3-(4-(1-(3-(trifluoromethyl)phenyl)-1H-1,2,4-triazol-3-yl)phenyl)urea (J323)

To 4-(1-(3-(trifluoromethyl)phenyl)-1H-1,2,4-triazol-3-yl)aniline (C524; 80 mg, 0.26 mmol) and bis(2,5-dioxopyrrolidin-1-yl) carbonate (80 mg, 0.32 mmol) in DCM (1.25 mL) was added pyridine (0.08 mL). The reaction mixture was stirred on an orbital shaker for 90 min. 2-Imino-3-(2-isopropyl-5-methylphenyl)thiazolidin-4-one (prepared as in U.S. Patent Application Publication 20140274688 A1; 78 mg, 0.32 mmol), water (0.5 mL), and sodium bicarbonate (221 mg, 2.6 mmol) were added, and the reaction mixture was stirred overnight. The reaction mixture was diluted with water and DCM and filtered through a phase separator onto a diatomaceous earth cartridge. Purification by flash chromatography eluting with 0-100% EtOAc-[1:1 DCM-hexanes] provided the title compound as an off-white solid (63 mg, 41%).

The following compounds were prepared in accordance to the procedure in Example 120:

### (Z)-1-(3-(2-Isopropyl-5-methoxyphenyl)-4-oxothiazolidin-2-ylidene)-3-(4-(1-(3-(trifluoromethyl)phenyl)-1H-1,2,4-triazol-3-yl)phenyl)urea (J324)

The title compound was prepared and was isolated as an off-white solid (101 mg, 63%).

### (Z)-1-(3-(5-(Dimethylamino)-2-isopropylphenyl)-4-oxothiazolidin-2-ylidene)-3-(4-(1-(3-(trifluoromethyl)phenyl)-1H-1,2,4-triazol-3-yl)phenyl)urea (J325)

The title compound was prepared and was isolated as an off-white solid (96 mg, 59%).

### (Z)-1-(3-(5-Methyl-2-(3,3,3-trifluoropropoxy)phenyl)-4-oxothiazolidin-2-ylidene)-3-(4-(1-(3-(trifluoromethyl)phenyl)-1H-1,2,4-triazol-3-yl)phenyl)urea (J326)

The title compound was prepared and was isolated as an off-white solid (110 mg, 63%).

### (Z)-1-(3-(2-Isopropyl-5-methylphenyl)-4-oxothiazolidin-2-ylidene)-3-(4-(1-(3-(trifluoromethoxy)phenyl)-1H-1,2,4-triazol-3-yl)phenyl)urea (J327)

The title compound was prepared and was isolated as an off-white solid (98 mg, 65%).

### (Z)-1-(3-(2-Isopropyl-5-methoxyphenyl)-4-oxothiazolidin-2-ylidene)-3-(4-(1-(3-(trifluoromethoxy)phenyl)-1H-1,2,4-triazol-3-yl)phenyl)urea (J328)

The title compound was prepared and was isolated as an off-white solid (71 mg, 46%).

### (Z)-1-(3-(5-(Dimethylamino)-2-isopropylphenyl)-4-oxothiazolidin-2-ylidene)-3-(4-(1-(3-(trifluoromethoxy)phenyl)-1H-1,2,4-triazol-3-yl)phenyl)urea (J329)

The title compound was prepared and was isolated as an off-white solid (57 mg, 36%).

### (Z)-1-(3-(5-Methyl-2-(3,3,3-trifluoropropoxy)phenyl)-4-oxothiazolidin-2-ylidene)-3-(4-(1-(3-(trifluoromethoxy)phenyl)-1H-1,2,4-triazol-3-yl)phenyl)urea (J330)

The title compound was prepared and was isolated as an off-white solid (28 mg, 17%).

### Example 121: Preparation of (Z)-1-(3-(2-(dimethylamino)-5-methylphenyl)-4-oxothiazolidin-2-ylidene)-3-(4-(1-(4-(trifluoromethoxy)phenyl)-1H-1,2,4-triazol-3-yl)phenyl)urea (J322)

(Z)-1-(3-(2-Amino-5-methylphenyl)-4-oxothiazolidin-2-ylidene)-3-(4-(1-(4-(trifluoromethoxy)phenyl)-1H-1,2,4-triazol-3-yl)phenyl)urea (J800; 0.100 g, 0.176 mmol) was suspended in methanol (1.762 mL). Formaldehyde (37% aq.; 0.066 mL, 0.881 mmol), acetic acid (0.025 mL, 0.440 mmol), and sodium cyanoborohydride (0.017 g, 0.264 mmol) were added. The solution became clear, and a precipitate began to form again over 1 h. The solvent was removed under a stream of nitrogen, and the material was loaded as a solid onto diatomaceous earth. Purification by silica gel chromatography eluting with 0-50% acetone-hexanes provided the title compound as a yellow powder (96 mg, 91%).

### Example 122: Preparation of (Z)-1-(3-(2-isopropyl-5-methylphenyl)-4-oxothiazolidin-2-ylidene)-3-(3-methyl-5-(1-(4-(trifluoromethyl)phenyl)-1H-1,2,4-triazol-3-yl)pyridin-2-yl)urea (J664)

To a solution of 2-imino-3-(2-isopropyl-5-methylphenyl)thiazolidin-4-one (prepared as in U.S. Patent Application Publication 20140274688 A1; 60 mg, 0.242 mmol) in dry THF (2 mL) was added 4-nitrophenyl carbonochloridate (49 mg, 0.242 mmol), and the reaction mixture was stirred at room temperature for 90 min during which a thick slurry formed. To the suspension were added 3-methyl-5-(1-(4-(trifluoromethyl)phenyl)-1H-1,2,4-triazol-3-yl)pyridin-2-amine (C345; 77 mg, 0.242 mmol), followed by N,N-diisopropylethylamine (0.127 mL, 0.725 mmol). The reaction mixture became homogenous and was warmed to 60° C. for 48 h. After cooling to room temperature, the reaction mixture was concentrated under a stream of nitrogen. Purification of the residue by silica gel column chromatography eluting with 0-100% EtOAc-hexanes gave the title compound as a red foam (18 mg, 12%).

The following compound was prepared in accordance to the procedure in Example 122:

### (Z)-1-(3-(5-Methyl-2-((2,2,2-trifluoroethoxy) methyl)phenyl)-4-oxothiazolidin-2-ylidene)-3-(3-methyl-5-(1-(4-(trifluoromethyl)phenyl)-1H-1,2,4-triazol-3-yl)pyridin-2-yl)urea (J707)

The title compound was prepared and was isolated as a light pink solid (32 mg, 16%).

### Example 123: Preparation of (Z)-1-(3-(2-amino-5-methylphenyl)-4-oxothiazolidin-2-ylidene)-3-(4-(1-(4-(trifluoromethoxy)phenyl)-1H-1,2,4-triazol-3-yl)phenyl)urea (J800)

(Z)-1-(3-(5-Methyl-2-nitrophenyl)-4-oxothiazolidin-2-ylidene)-3-(4-(1-(4-(trifluoromethoxy)phenyl)-1H-1,2,4-triazol-3-yl)phenyl)urea (J313; 0.560 g, 0.937 mmol) was suspended in ethanol (4.69 mL) and EtOAc (4.69 mL), and 10% palladium on carbon (0.020 g, 9.37 μmol) was added. The flask was evacuated and backfilled with hydrogen (balloon). The mixture stirred for 2 h at 23° C., and the mixture was filtered. Purification by chromatography eluting with 0-100% acetone-hexanes afforded the title compound as a tan solid (100 mg, 18%).

### Example 124: Preparation of (Z)-1-(2-fluoro-4-(5-(4-(trifluoromethoxy)phenyl)-1,3,4-oxadiazol-2-yl)phenyl)-3-(3-(2-isopropyl-5-methylphenyl)-4-oxothiazolidin-2-ylidene)urea (J607)

2-Fluoro-4-(5-(4-(trifluoromethoxy)phenyl)-1,3,4-oxadiazol-2-yl)aniline (C495; 50 mg, 0.147 mmol) was dissolved in THF (1 mL), and 4-nitrophenyl carbonochloridate (30 mg, 0.147 mmol) was added, causing the reaction to become cloudy. The reaction mixture was allowed to stir at room temperature overnight. 2-Imino-3-(2-isopropyl-5-methylphenyl)thiazolidin-4-one (37 mg, 0.147 mmol) was added, followed by diisopropylethylamine (77 μL, 0.442 mmol). The reaction mixture was allowed to proceed for another 40 minutes at room temperature, at which time LC-MS indicated reaction completion. The solvent was removed in vacuo, and the residue was taken up in DCM (10 mL), and washed with 20% weight per weight (w/w) aqueous sodium bicarbonate (2×5 mL). The organic layer was passed through a phase separator, and the solvent was removed in vacuo. The residue was taken up in a minimal amount of EtOAc, and heptanes was added, causing a solid to precipitate. The solid was collected by vacuum filtration to yield the title compound as a beige solid (43 mg, 45%), which required no further purification.

The following molecules were prepared in accordance to the procedure in Example 124:

### (Z)-1-(2-Fluoro-4-(5-(4-(trifluoromethoxy)phenyl)-1,3,4-oxadiazol-2-yl)phenyl)-3-(3-(5-methyl-2-propylphenyl)-4-oxothiazolidin-2-ylidene)urea (J608)

The title compound was prepared and was isolated as a light orange solid (35 mg, 33%).

### (Z)-1-(2-Fluoro-4-(5-(4-(trifluoromethoxy)phenyl)-1,3,4-oxadiazol-2-yl)phenyl)-3-(3-(2-(methoxymethyl)-5-methylphenyl)-4-oxothiazolidin-2-ylidene)urea (J609)

The title compound was prepared and was isolated as a light orange solid (41 mg, 45%).

### (Z)-1-(3-(2-(Ethoxymethyl)-5-methylphenyl)-4-oxothiazolidin-2-ylidene)-3-(2-fluoro-4-(5-(4-(trifluoromethoxy)phenyl)-1,3,4-oxadiazol-2-yl)phenyl)urea (J610)

The title compound was prepared and was isolated as a light pink solid (52 mg, 54%).

### (Z)-1-(3-(5-(Dimethylamino)-2-(trifluoromethyl)phenyl)-4-oxothiazolidin-2-ylidene)-3-(2-fluoro-4-(5-(4-(trifluoromethoxy)phenyl)-1,3,4-oxadiazol-2-yl)phenyl)urea (J611)

The title compound was prepared and was isolated as a light pink solid (56 mg, 54%).

### (Z)-1-(3-(5-(Dimethylamino)-2-(methoxymethyl)phenyl)-4-oxothiazolidin-2-ylidene)-3-(2-fluoro-4-(5-(4-(trifluoromethoxy)phenyl)-1,3,4-oxadiazol-2-yl)phenyl)urea (J612)

The title compound was prepared and was isolated as a light pink solid (50 mg, 65%).

### (Z)-1-(3-(2-Isopropyl-5-methylphenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J613)

The title compound was prepared and was isolated as a light grey solid (55 mg, 63%).

### (Z)-1-(3-(2-(1-Methoxyethyl)-5-methylphenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J614)

The title compound was prepared and was isolated as a tan solid (70 mg, 78%).

### (Z)-1-(3-(2-(Ethoxymethyl)-5-methylphenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J615)

The title compound was prepared and was isolated as a light pink solid (51 mg, 56%).

### (Z)-1-(3-(5-Methyl-2-((2,2,2-trifluoroethoxy)methyl)phenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J616)

The title compound was prepared and was isolated as a pink solid (64 mg, 66%).

### (Z)-1-(3-(5-Methyl-2-(2,2,2-trifluoro-1-methoxyethyl)phenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J617)

The title compound was prepared and was isolated as a light grey solid (76 mg, 75%).

### (Z)-1-(3-(5-(Dimethylamino)-2-isopropylphenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J618)

The title compound was prepared and was isolated as a tan solid (68 mg, 71%).

### (Z)-1-(3-(5-(Dimethylamino)-4-fluoro-2-isopropylphenyl)-4-oxothiazolidin-2-ylidene)-3-(2-methyl-4-(1-(4-(pentafluoro-λ⁶-sulfaneyl)phenyl)-1H-imidazol-4-yl)phenyl)urea (J619)

The title compound was prepared and was isolated as a beige solid (71 mg, 76%).

### (Z)-1-(2-Fluoro-4-(1-(4-((trifluoromethyl)sulfonyl)phenyl)-1H-imidazol-4-yl)phenyl)-3-(3-(2-(1-methoxyethyl)-5-methylphenyl)-4-oxothiazolidin-2-ylidene)urea (J645)

The title compound was prepared and was isolated as an off-white solid (56 mg, 62%).

### (Z)-1-(3-(2-(Ethoxymethyl)-5-methylphenyl)-4-oxothiazolidin-2-ylidene)-3-(2-fluoro-4-(1-(4-((trifluoromethyl)sulfonyl)phenyl)-1H-imidazol-4-yl)phenyl)urea (J646)

The title compound was prepared and was isolated as an off-white solid (52 mg, 57%).

### (Z)-1-(2-Fluoro-4-(1-(4-((trifluoromethyl)sulfonyl)phenyl)-1H-imidazol-4-yl)phenyl)-3-(3-(5-methyl-2-((2,2,2-trifluoroethoxy)methyl)phenyl)-4-oxothiazolidin-2-ylidene)urea (J647)

The title compound was prepared and was isolated as an off-white solid (60 mg, 61%).

### (Z)-1-(3-(2-((2,2-Difluoroethoxy)methyl)-5-methylphenyl)-4-oxothiazolidin-2-ylidene)-3-(2-fluoro-4-(1-(4-((trifluoromethyl)sulfonyl)phenyl)-1H-imidazol-4-yl)phenyl)urea (J648)

The title compound was prepared and was isolated as a yellow solid (59 mg, 62%).

### (Z)-1-(2-Fluoro-4-(1-(4-((trifluoromethyl)sulfonyl)phenyl)-1H-imidazol-4-yl)phenyl)-3-(3-(5-methyl-2-(2,2,2-trifluoro-1-methoxyethyl)phenyl)-4-oxothiazolidin-2-ylidene)urea (J649)

The title compound was prepared and was isolated as a light pink solid (66 mg, 67%).

### (Z)-1-(3-(5-(Dimethylamino)-2-isopropylphenyl)-4-oxothiazolidin-2-ylidene)-3-(2-fluoro-4-(1-(4-((trifluoromethyl)sulfonyl)phenyl)-1H-imidazol-4-yl)phenyl)urea (J650)

The title compound was prepared and was isolated as a beige solid (66 mg, 72%).

### (Z)-1-(2-Fluoro-4-(1-(4-((trifluoromethyl)sulfonyl)phenyl)-1H-imidazol-4-yl)phenyl)-3-(3-(2-isopropyl-5-methylphenyl)-4-oxothiazolidin-2-ylidene)urea (J651)

The title compound was prepared and was isolated as a beige solid (51 mg, 58%).

### (Z)-1-(3-(5-Cyano-2-(1-methoxyethyl)phenyl)-4-oxothiazolidin-2-ylidene)-3-(2-fluoro-4-(1-(4-((trifluoromethyl)sulfonyl)phenyl)-1H-imidazol-4-yl)phenyl)urea (J652)

The title compound was prepared and was isolated as a pink solid (60 mg, 64%).

### (Z)-1-(2-Fluoro-4-(1-(4-((trifluoromethyl)sulfonyl)phenyl)-1H-1,2,4-triazol-3-yl)phenyl)-3-(3-(5-methyl-2-(2,2,2-trifluoro-1-methoxyethyl)phenyl)-4-oxothiazolidin-2-ylidene)urea (J666)

The title compound was prepared and was isolated as a pink solid (63.5 mg, 60%).

### (Z)-1-(2-Fluoro-4-(1-(4-((trifluoromethyl)sulfonyl)phenyl)-1H-1,2,4-triazol-3-yl)phenyl)-3-(3-(2-isopropyl-5-methylphenyl)-4-oxothiazolidin-2-ylidene)urea (J667)

The title compound was prepared and was isolated as a beige solid (40 mg, 44%).

### (Z)-1-(2-Fluoro-4-(1-(4-((trifluoromethyl)sulfonyl)phenyl)-1H-1,2,4-triazol-3-yl)phenyl)-3-(3-(2-((2-fluoroethoxy)methyl)-5-methylphenyl)-4-oxothiazolidin-2-ylidene)urea (J668)

The title compound was prepared and was isolated as a beige solid (53 mg, 58%).

### (Z)-1-(2-Fluoro-4-(1-(4-((trifluoromethyl)sulfonyl)phenyl)-1H-1,2,4-triazol-3-yl)phenyl)-3-(3-(5-methoxy-2-((2,2,2-trifluoroethoxy)methyl)phenyl)-4-oxothiazolidin-2-ylidene)urea (J669)

The title compound was prepared and was isolated as a light pink solid (62 mg, 61%).

### (Z)-1-(3-(5-Fluoro-2-((2,2,2-trifluoroethoxy)methyl)phenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J670)

The title compound was prepared and was isolated as a light pink solid (54 mg, 56%).

### (Z)-1-(3-(5-(Dimethylamino)-2-(methoxymethyl)phenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J671)

The title compound was prepared and was isolated as a beige solid (52 mg, 57%).

### (Z)-1-(3-(5-(Dimethylamino)-2-(2,2,2-trifluoroethoxy)phenyl)-4-oxothiazolidin-2-ylidene)-3-(2-fluoro-4-(1-(4-((trifluoromethyl)sulfonyl)phenyl)-1H-1,2,4-triazol-3-yl)phenyl)urea (J672)

The title compound was prepared and was isolated as a pink solid (56 mg, 55%).

### (Z)-1-(2-Fluoro-4-(3-(4-(trifluoromethoxy)phenyl)-1H-1,2,4-triazol-1-yl)phenyl)-3-(3-(2-(1-methoxyethyl)-5-methylphenyl)-4-oxothiazolidin-2-ylidene)urea (J685)

The title compound was prepared and was isolated as a light pink solid (42 mg, 42%).

### (Z)-1-(2-Fluoro-4-(3-(4-(trifluoromethoxy)phenyl)-1H-1,2,4-triazol-1-yl)phenyl)-3-(3-(5-methyl-2-(2,2,2-trifluoro-1-methoxyethyl)phenyl)-4-oxothiazolidin-2-ylidene)urea (J687)

The title compound was prepared and was isolated as a pink solid (45 mg, 43%).

### (Z)-1-(3-(5-(Dimethylamino)-2-(2,2,2-trifluoroethoxy)phenyl)-4-oxothiazolidin-2-ylidene)-3-(2-fluoro-4-(3-(4-(trifluoromethoxy)phenyl)-1H-1,2,4-triazol-1-yl)phenyl)urea (J688)

The title compound was prepared and was isolated as a pink solid (56 mg, 49%).

### (Z)-1-(2-Fluoro-4-(3-(4-(trifluoromethoxy)phenyl)-1H-1,2,4-triazol-1-yl)phenyl)-3-(3-(5-methyl-2-((2,2,2-trifluoroethoxy)methyl)phenyl)-4-oxothiazolidin-2-ylidene)urea (J689)

The title compound was prepared and was isolated as a light pink solid (43 mg, 41%).

### (Z)-1-(3-(5-Fluoro-2-((2,2,2-trifluoroethoxy)methyl)phenyl)-4-oxothiazolidin-2-ylidene)-3-(2-fluoro-4-(3-(4-(trifluoromethoxy)phenyl)-1H-1,2,4-triazol-1-yl)phenyl)urea (J690)

The title compound was prepared and was isolated as a pink solid (67 mg, 59%).

### (Z)-1-(2-Fluoro-4-(3-(4-(trifluoromethoxy)phenyl)-1H-1,2,4-triazol-1-yl)phenyl)-3-(3-(5-methoxy-2-((2,2,2-trifluoroethoxy)methyl)phenyl)-4-oxothiazolidin-2-ylidene)urea (J691)

The title compound was prepared and was isolated as a pink solid (68 mg, 62%).

### (Z)-1-(3-(2-((2,2-Difluoroethoxy)methyl)-5-methylphenyl)-4-oxothiazolidin-2-ylidene)-3-(2-fluoro-4-(3-(4-(trifluoromethoxy)phenyl)-1H-1,2,4-triazol-1-yl)phenyl)urea (J692)

The title compound was prepared and was isolated as a pink solid (37 mg, 36%).

### (Z)-1-(2-Fluoro-4-(3-(4-(trifluoromethoxy)phenyl)-1H-1,2,4-triazol-1-yl)phenyl)-3-(3-(5-methyl-2-(3,3,3-trifluoropropoxy)phenyl)-4-oxothiazolidin-2-ylidene)urea (J693)

The title compound was prepared and was isolated as a beige solid (60 mg, 57%).

### (Z)-1-(2-Fluoro-4-(3-(4-(trifluoromethyl)phenyl)-1H-1,2,4-triazol-1-yl)phenyl)-3-(3-(2-isopropyl-5-methylphenyl)-4-oxothiazolidin-2-ylidene)urea (J694)

The title compound was prepared and was isolated as a dark pink solid (31 mg, 30%).

### (Z)-1-(2-Fluoro-4-(3-(4-(trifluoromethyl)phenyl)-1H-1,2,4-triazol-1-yl)phenyl)-3-(3-(2-(1-methoxyethyl)-5-methylphenyl)-4-oxothiazolidin-2-ylidene)urea (J695)

The title compound was prepared and was isolated as a light pink solid (40 mg, 40%).

### (Z)-1-(2-Fluoro-4-(3-(4-(trifluoromethyl)phenyl)-1H-1,2,4-triazol-1-yl)phenyl)-3-(3-(5-methyl-2-(2,2,2-trifluoro-1-methoxyethyl)phenyl)-4-oxothiazolidin-2-ylidene)urea (J696)

The title compound was prepared and was isolated as a pink solid (47 mg, 43%).

### (Z)-1-(3-(5-(Dimethylamino)-2-(2,2,2-trifluoroethoxy)phenyl)-4-oxothiazolidin-2-ylidene)-3-(2-fluoro-4-(3-(4-(trifluoromethyl)phenyl)-1H-1,2,4-triazol-1-yl)phenyl)urea (J697)

The title compound was prepared and was isolated as a pink solid (63 mg, 57%).

### (Z)-1-(2-fluoro-4-(3-(4-(trifluoromethyl)phenyl)-1H-1,2,4-triazol-1-yl)phenyl)-3-(3-(5-methyl-2-((2,2,2-trifluoroethoxy)methyl)phenyl)-4-oxothiazolidin-2-ylidene)urea (J698)

The title compound was prepared and was isolated as a light pink solid (36 mg, 34%).

### (Z)-1-(3-(5-Fluoro-2-((2,2,2-trifluoroethoxy)methyl)phenyl)-4-oxothiazolidin-2-ylidene)-3-(2-fluoro-4-(3-(4-(trifluoromethyl)phenyl)-1H-1,2,4-triazol-1-yl)phenyl)urea (J699)

The title compound was prepared and was isolated as a pink solid (51 mg, 48%).

### (Z)-1-(2-Fluoro-4-(3-(4-(trifluoromethyl)phenyl)-1H-1,2,4-triazol-1-yl)phenyl)-3-(3-(5-methoxy-2-((2,2,2-trifluoroethoxy)methyl)phenyl)-4-oxothiazolidin-2-ylidene)urea (J700)

The title compound was prepared and was isolated as a pink solid (67 mg, 60%).

### (Z)-1-(3-(2-((2,2-Difluoroethoxy)methyl)-5-methylphenyl)-4-oxothiazolidin-2-ylidene)-3-(2-fluoro-4-(3-(4-(trifluoromethyl)phenyl)-1H-1,2,4-triazol-1-yl)phenyl)urea (J701)

The title compound was prepared and was isolated as a light pink solid (35 mg, 34%).

### (Z)-1-(2-Fluoro-4-(3-(4-(trifluoromethyl)phenyl)-1H-1,2,4-triazol-1-yl)phenyl)-3-(3-(5-methyl-2-(3,3,3-trifluoropropoxy)phenyl)-4-oxothiazolidin-2-ylidene)urea (J702)

The title compound was prepared and was isolated as a beige solid (51 mg, 48%).

### (Z)-1-(2-Fluoro-4-(3-(4-(trifluoromethoxy)phenyl)-1H-1,2,4-triazol-1-yl)phenyl)-3-(3-(3-methyl-2-((2,2,2-trifluoroethoxy)methyl)phenyl)-4-oxothiazolidin-2-ylidene)urea (J703)

The title compound was prepared and was isolated as a light pink solid (44 mg, 41%).

### (Z)-1-(2-Fluoro-4-(3-(4-(trifluoromethyl)phenyl)-1H-1,2,4-triazol-1-yl)phenyl)-3-(3-(3-methyl-2-((2,2,2-trifluoroethoxy)methyl)phenyl)-4-oxothiazolidin-2-ylidene)urea (J704)

The title compound was prepared and was isolated as a beige solid (53 mg, 50%).

### (Z)-1-(2-Chloro-4-(3-(4-(trifluoromethoxy)phenyl)-1H-1,2,4-triazol-1-yl)phenyl)-3-(3-(2-isopropyl-5-methylphenyl)-4-oxothiazolidin-2-ylidene)urea (J728)

The title compound was prepared and was isolated as an off-white solid (40 mg, 54%).

### (Z)-1-(2-Chloro-4-(3-(4-(trifluoromethoxy)phenyl)-1H-1,2,4-triazol-1-yl)phenyl)-3-(3-(5-methyl-2-(2,2,2-trifluoro-1-methoxyethyl)phenyl)-4-oxothiazolidin-2-ylidene)urea (J729)

The title compound was prepared and was isolated as a pink solid (29 mg, 35%).

### (Z)-1-(2-Chloro-4-(3-(4-(trifluoromethoxy)phenyl)-1H-1,2,4-triazol-1-yl)phenyl)-3-(3-(2-(methoxymethyl)-5-methylphenyl)-4-oxothiazolidin-2-ylidene)urea (J730)

The title compound was prepared and was isolated as a light orange solid (46 mg, 62%).

### (Z)-1-(2-Chloro-4-(3-(4-(trifluoromethoxy)phenyl)-1H-1,2,4-triazol-1-yl)phenyl)-3-(3-(5-methyl-2-((2,2,2-trifluoroethoxy)methyl)phenyl)-4-oxothiazolidin-2-ylidene)urea (J731)

The title compound was prepared and was isolated as a pink solid (50 mg, 60%).

### (Z)-1-(2-Chloro-4-(3-(4-(trifluoromethoxy)phenyl)-1H-1,2,4-triazol-1-yl)phenyl)-3-(4-oxo-3-(2-((2,2,2-trifluoroethoxy)methyl)phenyl)thiazolidin-2-ylidene)urea (J732)

The title compound was prepared and was isolated as a light pink solid (47 mg, 58%).

### (Z)-1-(2-Chloro-4-(3-(4-(trifluoromethoxy)phenyl)-1H-1,2,4-triazol-1-yl)phenyl)-3-(3-(4,5-dimethyl-2-((2,2,2-trifluoroethoxy)methyl)phenyl)-4-oxothiazolidin-2-ylidene)urea (J733)

The title compound was prepared and was isolated as a tan solid (51 mg, 60%).

### (Z)-1-(2-Chloro-4-(3-(4-(trifluoromethoxy)phenyl)-1H-1,2,4-triazol-1-yl)phenyl)-3-(3-(4-methyl-2-((2,2,2-trifluoroethoxy)methyl)phenyl)-4-oxothiazolidin-2-ylidene)urea (J734)

The title compound was prepared and was isolated as a red solid (511 mg, 61%).

### (Z)-1-(2-chloro-4-(3-(4-(trifluoromethoxy)phenyl)-1H-1,2,4-triazol-1-yl)phenyl)-3-(3-(3-methyl-2-((2,2,2-trifluoroethoxy)methyl)phenyl)-4-oxothiazolidin-2-ylidene)urea (J735)

The title compound was prepared and was isolated as a pink solid (51 mg, 61%).

### (Z)-1-(2-Chloro-4-(3-(4-(trifluoromethoxy)phenyl)-1H-1,2,4-triazol-1-yl)phenyl)-3-(3-(2-((2,2-difluoroethoxy)methyl)-5-methylphenyl)-4-oxothiazolidin-2-ylidene)urea (J736)

The title compound was prepared and was isolated as a light orange solid (46 mg, 60%).

### (Z)-1-(2-Chloro-4-(3-(4-(trifluoromethoxy)phenyl)-1H-1,2,4-triazol-1-yl)phenyl)-3-(3-(5-methyl-2-(3,3,3-trifluoropropoxy)phenyl)-4-oxothiazolidin-2-ylidene)urea (J737)

The title compound was prepared and was isolated as a dark orange solid (43 mg, 52%).

### (Z)-1-(3-(5-Methyl-2-((2,2,2-trifluoroethoxy)methyl)phenyl)-4-oxothiazolidin-2-ylidene)-3-(4-(2-(4-(trifluoromethoxy)phenyl)-2H-tetrazol-5-yl)phenyl)urea (J767)

The title compound was prepared and was isolated as a light pink solid (36 mg, 47%).

### (Z)-1-(3-(2-((2,2-Difluoroethoxy)methyl)-5-methylphenyl)-4-oxothiazolidin-2-ylidene)-3-(4-(2-(4-(trifluoromethoxy)phenyl)-2H-tetrazol-5-yl)phenyl)urea (J768)

The title compound was prepared and was isolated as a light pink solid (33 mg, 43%).

### (Z)-1-(2-Fluoro-4-(5-(4-(trifluoromethoxy)phenyl)-1,2,4-oxadiazol-3-yl)phenyl)-3-(3-(5-methyl-2-((2,2,2-trifluoroethoxy)methyl)phenyl)-4-oxothiazolidin-2-ylidene)urea (J770)

The title compound was prepared and was isolated as a light pink solid (19 mg, 26%).

### (Z)-1-(3-(2-((2,2-Difluoroethoxy)methyl)-5-methylphenyl)-4-oxothiazolidin-2-ylidene)-3-(2-fluoro-4-(5-(4-(trifluoromethoxy)phenyl)-1,2,4-oxadiazol-3-yl)phenyl)urea (J771)

The title compound was prepared and was isolated as a pink solid (42 mg, 57%).

### (Z)-1-(2-Fluoro-4-(5-(4-(trifluoromethoxy)phenyl)-1,2,4-oxadiazol-3-yl)phenyl)-3-(3-(2-(methoxymethyl)-5-methylphenyl)-4-oxothiazolidin-2-ylidene)urea (J772)

The title compound was prepared and was isolated as a light pink solid (17 mg, 25%).

### (Z)-1-(3-(5-(Dimethylamino)-2-(methoxymethyl)phenyl)-4-oxothiazolidin-2-ylidene)-3-(2-fluoro-4-(5-(4-(trifluoromethoxy)phenyl)-1,2,4-oxadiazol-3-yl)phenyl)urea (J773)

The title compound was prepared and was isolated as a beige solid (41 mg, 58%).

### (Z)-1-(2-Chloro-4-(4-(4-(trifluoromethoxy)phenyl)-1H-imidazol-1-yl)phenyl)-3-(3-(5-methyl-2-((2,2,2-trifluoroethoxy)methyl)phenyl)-4-oxothiazolidin-2-ylidene)urea (J786)

The title compound was prepared and was isolated as a pink solid (21 mg, 24%).

### (Z)-1-(2-Chloro-4-(4-(4-(trifluoromethoxy)phenyl)-1H-imidazol-1-yl)phenyl)-3-(3-(2-((2,2-difluoroethoxy)methyl)-5-methylphenyl)-4-oxothiazolidin-2-ylidene)urea (J787)

The title compound was prepared and was isolated as a light pink solid (25 mg, 30%).

### (Z)-1-(2-Chloro-4-(4-(4-(trifluoromethoxy)phenyl)-1H-imidazol-1-yl)phenyl)-3-(3-(2-(methoxymethyl)-5-methylphenyl)-4-oxothiazolidin-2-ylidene)urea (J788)

The title compound was prepared and was isolated as a light pink solid (19 mg, 24%).

### (Z)-1-(2-Chloro-4-(4-(4-(trifluoromethoxy)phenyl)-1H-imidazol-1-yl)phenyl)-3-(3-(5-(dimethylamino)-2-(methoxymethyl)phenyl)-4-oxothiazolidin-2-ylidene)urea (J789)

The title compound was prepared and was isolated as a light pink solid (37 mg, 48%).

### (Z)-1-(2-Chloro-4-(4-(4-(trifluoromethoxy)phenyl)-1H-imidazol-1-yl)phenyl)-3-(3-(5-methyl-2-(3,3,3-trifluoropropoxy)phenyl)-4-oxothiazolidin-2-ylidene)urea (J790)

The title compound was prepared and was isolated as a beige solid (31 mg, 37%).

### (Z)-1-(2-Chloro-4-(4-(4-(trifluoromethoxy)phenyl)-1H-imidazol-1-yl)phenyl)-3-(3-(5-methyl-2-(2,2,2-trifluoro-1-methoxyethyl)phenyl)-4-oxothiazolidin-2-ylidene)urea (J791)

The title compound was prepared and was isolated as a pink solid (32 mg, 37%).

### (Z)-1-(2-Chloro-4-(4-(4-(trifluoromethoxy)phenyl)-1H-imidazol-1-yl)phenyl)-3-(3-(2-isopropyl-5-methylphenyl)-4-oxothiazolidin-2-ylidene)urea (J792)

The title compound was prepared and was isolated as a beige solid (12 mg, 15%).

### (Z)-1-(3-(2-((2-Fluoroethoxy)methyl)-5-methylphenyl)-4-oxothiazolidin-2-ylidene)-3-(2-methyl-4-(1-(4-(perfluoroethoxy)phenyl)-1H-1,2,4-triazol-3-yl)phenyl)urea (J801)

The title compound was prepared and was isolated as a light pink solid (54 mg, 57%).

### (Z)-1-(3-(5-Methyl-2-((2,2,2-trifluoroethoxy)methyl)phenyl)-4-oxothiazolidin-2-ylidene)-3-(4-(5-phenyl-1H-1,2,4-triazol-3-yl)phenyl)urea (J802)

The title compound was prepared and was isolated as a beige solid (54 mg, 59%).

### Example 125: Preparation of (Z)-1-(2-fluoro-4-(1-(4-(trifluoromethoxy)phenyl)-1H-1,2,4-triazol-3-yl)phenyl)-3-(3-(2-(((3-fluorobenzyl)oxy)methyl)-5-methylphenyl)-4-oxothiazolidin-2-ylidene)urea (J568)

A solution of 2-fluoro-4-(1-(4-(trifluoromethoxy)phenyl)-1H-1,2,4-triazol-3-yl)aniline (39.3 mg, 0.116 mmol) in DCM (2 mL) was added dropwise over the course of 10 minutes to a solution of bis(trichloromethyl) carbonate (13.8 mg, 0.046 mmol) and N,N-diisopropylethylamine (60.7 μL, 0.348 mmol) in DCM (3 mL). The resulting reaction mixture was stirred at room temperature for 30 minutes. 3-(2-(((3-Fluorobenzyl)oxy)methyl)-5-methylphenyl)-2-iminothiazolidin-4-one (40 mg, 0.116 mmol) in DCM (2 mL) was added in one portion via syringe. The solvent was removed in vacuo to afford a residue. Purification of the residue by flash chromatography eluting with 0-30% EtOAc-Hexanes provided the title compound as a yellow oil (69 mg, 80%).

The following molecules were prepared in accordance to the procedure in Example 125:

### (Z)-1-(2-Fluoro-4-(1-(4-(trifluoromethoxy)phenyl)-1H-1,2,4-triazol-3-yl)phenyl)-3-(3-(5-methyl-2-(((4-(trifluoromethoxy)benzyl)oxy)methyl)phenyl)-4-oxothiazolidin-2-ylidene)urea (J570)

The title compound was prepared and was isolated as a yellow oil (76 mg, 91%).

### (Z)-1-(3-(2-((Benzyloxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J571)

The title compound was prepared and was isolated as a yellow oil (61 mg, 68%).

### (Z)-1-(3-(2-(((2-Chlorobenzyl)oxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J572)

The title compound was prepared and was isolated as a yellow oil (69 mg, 81%).

### (Z)-1-(3-(2-(((2,4-Difluorobenzyl)oxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J573)

The title compound was prepared and was isolated as a yellow oil (59 mg, 70%).

### (Z)-1-(2-Fluoro-4-(1-(4-(trifluoromethoxy)phenyl)-1H-1,2,4-triazol-3-yl)phenyl)-3-(3-(2-(((4-fluorobenzyl)oxy)methyl)-5-methylphenyl)-4-oxothiazolidin-2-ylidene)urea (J574)

The title compound was prepared and was isolated as a yellow oil (65 mg, 75%).

### (Z)-1-(3-(2-(((4-Fluoro-2-(trifluoromethyl)benzyl)oxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J621)

The title compound was prepared and was isolated as a yellow oil (67 mg, 78%).

### (Z)-1-(2-Fluoro-4-(1-(4-(trifluoromethoxy)phenyl)-1H-1,2,4-triazol-3-yl)phenyl)-3-(3-(5-methyl-2-(((perfluorophenyl)methoxy)methyl)phenyl)-4-oxothiazolidin-2-ylidene)urea (J622)

The title compound was prepared and was isolated as a white solid (52 mg, 80%).

### (Z)-1-(2-Fluoro-4-(1-(4-(trifluoromethoxy)phenyl)-1H-1,2,4-triazol-3-yl)phenyl)-3-(3-(5-methyl-2-((thiophen-3-ylmethoxy)methyl)phenyl)-4-oxothiazolidin-2-ylidene)urea (J623)

The title compound was prepared and was isolated as a yellow oil (75 mg, 70%).

### (Z)-1-(3-(2-((Cyclopropylmethoxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J624)

The title compound was prepared and was isolated as a white solid (41 mg, 67%).

### (Z)-1-(2-Fluoro-4-(1-(4-(trifluoromethoxy)phenyl)-1H-1,2,4-triazol-3-yl)phenyl)-3-(3-(2-(((2-fluorobenzyl)oxy)methyl)-5-methylphenyl)-4-oxothiazolidin-2-ylidene)urea (J635)

The title compound was prepared and was isolated as a yellow oil (41 mg, 42%).

### (Z)-1-(3-(2-((Benzyloxy)methyl)-4-chlorophenyl)-4-oxothiazolidin-2-ylidene)-3-(2-fluoro-4-(1-(4-(trifluoromethoxy)phenyl)-1H-1,2,4-triazol-3-yl)phenyl)urea (J636)

The title compound was prepared and was isolated as a white solid (44 mg, 41%).

### (Z)-1-(3-(2-((Benzo[d]thiazol-2-ylmethoxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J640)

The title compound was prepared and was isolated as a white solid (21 mg, 20.5%).

### (Z)-1-(2-Fluoro-4-(1-(4-(trifluoromethoxy)phenyl)-1H-1,2,4-triazol-3-yl)phenyl)-3-(3-(5-methyl-2-(((1-methyl-3-(trifluoromethyl)-1H-thieno[2,3-c]pyrazol-5-yl)methoxy)methyl)phenyl)-4-oxothiazolidin-2-ylidene)urea (J712)

The title compound was prepared and was isolated as a orange solid (103 mg, 91%).

### (Z)-1-(3-(4-Fluoro-2,5-dimethylphenyl)-4-oxothiazolidin-2-ylidene)-3-(2-fluoro-4-(1-(4-(trifluoromethoxy)phenyl)-1H-1,2,4-triazol-3-yl)phenyl)urea (J714)

The title compound was prepared and was isolated as a yellow solid (134 mg, 33%).

### (Z)-1-(3-([1,1′-Biphenyl]-3-yl)-4-oxothiazolidin-2-ylidene)-3-(2-fluoro-4-(1-(4-(trifluoromethoxy)phenyl)-1H-1,2,4-triazol-3-yl)phenyl)urea (J715)

The title compound was prepared and was isolated as a yellow solid (98 mg, 62%).

### (Z)-1-(3-(2-((Benzyloxy)methyl)-5-chlorophenyl)-4-oxothiazolidin-2-ylidene)-3-(2-fluoro-4-(1-(4-(trifluoromethoxy)phenyl)-1H-1,2,4-triazol-3-yl)phenyl)urea (J719)

The title compound was prepared and was isolated as a yellow solid (94 mg, 86%).

### (Z)-1-(3-([1,1′-Biphenyl]-2-yl)-4-oxothiazolidin-2-ylidene)-3-(2-fluoro-4-(1-(4-(trifluoromethoxy)phenyl)-1H-1,2,4-triazol-3-yl)phenyl)urea (J720)

The title compound was prepared and was isolated as a yellow solid (82 mg, 55%).

### (Z)-1-(3-(2-((Benzyloxy)methyl)-5-bromophenyl)-4-oxothiazolidin-2-ylidene)-3-(2-fluoro-4-(1-(4-(trifluoromethoxy)phenyl)-1H-1,2,4-triazol-3-yl)phenyl)urea (J740)

The title compound was prepared and was isolated as a yellow solid (78 mg, 96%).

### (Z)-1-(3-(2-((Benzyloxy)methyl)-5-(trifluoromethyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J741)

The title compound was prepared and was isolated as a yellow oil (54 mg, 44%).

### (Z)-1-(3-(2-((Benzyloxy)methyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J742)

The title compound was prepared and was isolated as a yellow solid (82 mg, 85%).

### (Z)-1-(3-([1,1′-Biphenyl]-4-yl)-4-oxothiazolidin-2-ylidene)-3-(2-fluoro-4-(1-(4-(trifluoromethoxy)phenyl)-1H-1,2,4-triazol-3-yl)phenyl)urea (J744)

The title compound was prepared and was isolated as a white solid (56 mg, 53%).

### (Z)-1-(3-(2-((Benzo[d]oxazol-2-ylmethoxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J745)

The title compound was prepared and was isolated as a yellow solid (32 mg, 36%).

### (Z)-1-(2-Fluoro-4-(1-(4-(trifluoromethyl)phenyl)-1H-1,2,4-triazol-3-yl)phenyl)-3-(3-(5-methyl-2-(((2-(trifluoromethoxy)benzyl)oxy)methyl)phenyl)-4-oxothiazolidin-2-ylidene)urea (J746)

The title compound was prepared and was isolated as a yellow solid (29 mg, 37%).

### (Z)-1-(3-(2-((Benzyloxy)methyl)-5-methylphenyl)-4-oxothiazolidin-2-ylidene)-3-(2-fluoro-4-(1-(4-(trifluoromethyl)phenyl)-1H-1,2,4-triazol-3-yl)phenyl)urea (J747)

The title compound was prepared and was isolated as a yellow solid (32 mg, 37%).

### (Z)-1-(3-(2-(((3,5-Bis(trifluoromethyl)benzyl)oxy)methyl)-5-methylphenyl)-4-oxothiazolidin-2-ylidene)-3-(2-fluoro-4-(1-(4-(trifluoromethyl)phenyl)-1H-1,2,4-triazol-3-yl)phenyl)urea (J748)

The title compound was prepared and was isolated as a yellow solid (34 mg, 46%).

### (Z)-1-(3-(2-(((2-Chlorobenzyl)oxy)methyl)-5-methylphenyl)-4-oxothiazolidin-2-ylidene)-3-(2-fluoro-4-(1-(4-(trifluoromethyl)phenyl)-1H-1,2,4-triazol-3-yl)phenyl)urea (J749)

The title compound was prepared and was isolated as a yellow oil (29 mg, 35%).

### (Z)-1-(3-(2-(((3,5-Bis(trifluoromethyl)benzyl)oxy)methyl)-5-methylphenyl)-4-oxothiazolidin-2-ylidene)-3-(2-fluoro-4-(1-(4-(trifluoromethoxy)phenyl)-1H-1,2,4-triazol-3-yl)phenyl)urea (J774)

The title compound was prepared and was isolated as a yellow solid (44 mg, 53%).

### (Z)-1-(2-Fluoro-4-(1-(4-(trifluoromethoxy)phenyl)-1H-1,2,4-triazol-3-yl)phenyl)-3-(3-(4-methyl-[1,1′-biphenyl]-2-yl)-4-oxothiazolidin-2-ylidene)urea (J785)

The title compound was prepared and was isolated as a yellow oil (39 mg, 35%).

### (Z)-1-(2-Fluoro-4-(1-(4-(trifluoromethoxy)phenyl)-1H-1,2,4-triazol-3-yl)phenyl)-3-(4-oxo-3-(2,3,5-trimethylphenyl)thiazolidin-2-ylidene)urea (J797)

The title compound was prepared and was isolated as a yellow oil (36 mg, 30%).

### (Z)-1-(2-Fluoro-4-(1-(4-(trifluoromethoxy)phenyl)-1H-1,2,4-triazol-3-yl)phenyl)-3-(3-(2-methoxy-3-methylphenyl)-4-oxothiazolidin-2-ylidene)urea (J798)

The title compound was prepared and was isolated as a yellow oil (27 mg, 23%).

### (Z)-1-(2-Fluoro-4-(1-(4-(trifluoromethoxy)phenyl)-1H-1,2,4-triazol-3-yl)phenyl)-3-(3-(2-methyl-5-(trifluoromethyl)phenyl)-4-oxothiazolidin-2-ylidene)urea (J799)

The title compound was prepared and was isolated as a yellow oil (35 mg, 33%).

### Example 126: Preparation of (Z)-1-(5,5-dichloro-3-(5-methyl-2-((2,2,2-trifluoroethoxy)methyl)phenyl)-4-oxothiazolidin-2-ylidene)-3-(2-fluoro-4-(1-(4-(trifluoromethoxy)phenyl)-1H-1,2,4-triazol-3-yl)phenyl)urea (J458)

To a vial containing (Z)-1-(2-fluoro-4-(1-(4-(trifluoromethoxy)phenyl)-1H-1,2,4-triazol-3-yl)phenyl)-3-(3-(5-methyl-2-((2,2,2-trifluoroethoxy)methyl)phenyl)-4-oxothiazolidin-2-ylidene)urea (J123; 100 mg, 0.147 mmol) and 1-chloropyrrolidine-2,5-dione (43.0 mg, 0.322 mmol) was added wet DMF (5.38 mL) and the reaction mixture was stirred at 40° C. The reaction mixture was monitored and a total of 10 molar equivalents (1.47 mmol) of 1-chloropyrrolidine-2,5-dione were added. The reaction mixture was diluted with MTBE (˜15 mL) and water (˜10 mL). The layers were separated, and the aqueous layer was extracted with DCM (10 mL). The combined organic layers were dried over Na₂SO₄, filtered, and concentrated to ˜3 mL, then loaded onto a silica cartridge. Purification by flash chromatography (silica gel) eluting with 0-100% EtOAc-1:1 hexanes/DCM yielded the title compound as a yellow solid (52 mg, 46%).

### Example 127: Preparation of 3-(2-(((3-fluorobenzyl)oxy)methyl)-5-methylphenyl)-2-iminothiazolidin-4-one (C528)

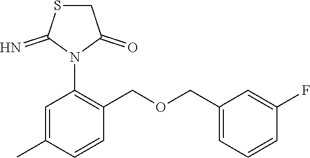 [SMILES: Cc1ccc(COCc2cccc(F)c2)c(N2C(=N)SCC2=O)c1]

2-Chloro-N-(2-(((3-fluorobenzyl)oxy)methyl)-5-methylphenyl)acetamide (C558; 100 mg, 0.311 mmol) and potassium thiocyanate (30.2 mg, 0.311 mmol) were added to a dried 10-mL vial equipped with a Teflon-coated stirbar and capped with a rubber septum. The vial was evacuated and backfilled with N₂ three times. Then, degassed and dry acetone (10 mL) was then added by syringe, and the resulting reaction mixture was refluxed for 12 h. Then, the reaction mixture was cooled to room temperature, and Cs₂CO₃ (253 mg, 0.777 mmol) was added in one portion and the resulting reaction mixture was stirred for 10 min at room temperature. At this point, the reaction turned red. The reaction mixture was filtered over diatomaceous earth, and concentrated in vacuo. Purification of the residue by flash chromatography eluting with 0-60% EtOAc-Hexanes provided the title compound as a yellow oil (88 mg, 78%): ¹H NMR (400 MHz, CDCl₃) δ 7.43 (d, J=7.8 Hz, 1H), 7.33-7.27 (m, 2H), 7.12-7.01 (m, 3H), 7.01-6.91 (m, 1H), 4.51-4.33 (m, 4H), 4.07-3.84 (m, 2H), 2.40 (s, 3H) ppm; ¹³C NMR (101 MHz, CDCl₃) δ 163.67, 161.23, 130.46, 129.80, 129.46, 129.41, 129.37, 122.65, 122.62, 114.10, 113.92, 113.89, 70.91, 68.52, 20.60 ppm; ¹⁹F NMR (376 MHz, CDCl₃) δ −113.20.

The following compounds were prepared according to the procedure described in Example 127:

### 2-Imino-3-(5-methyl-2-(((2-(trifluoromethoxy)benzyl)oxy)methyl)phenyl)thiazolidin-4-one (C529)

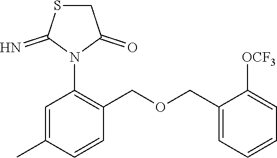 [SMILES: Cc1ccc(COCc2ccccc2OC(F)(F)F)c(N2C(=N)SCC2=O)c1]

The title compound was prepared and was isolated as a yellow oil (102 mg, 87%): ¹H NMR (500 MHz, CDCl₃) δ 7.78 (s, 1H), 7.49-7.27 (m, 4H), 7.18 (d, J=8.0 Hz, 2H), 7.03 (s, 1H), 4.58-4.34 (m, 4H), 4.00 (d, J=16.8 Hz, 1H), 3.87 (d, J=16.6 Hz, 1H), 2.40 (s, 3H); ¹⁹F NMR (471 MHz, CDCl₃) δ −57.90.

### 3-(2-(((2,4-Difluorobenzyl)oxy)methyl)-5-methylphenyl)-2-iminothiazolidin-4-one (C530)

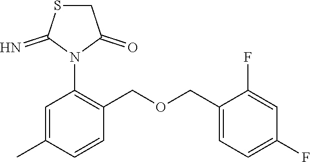 [SMILES: Cc1ccc(COCc2ccc(F)cc2F)c(N2C(=N)SCC2=O)c1]

The title compound was prepared and was isolated as a yellow oil (104 mg, 88%): ¹H NMR (400 MHz, CDCl₃) δ 7.79 (s, 1H), 7.36 (tq, J=24.3, 8.0 Hz, 3H), 7.02 (s, 1H), 6.91-6.73 (m, 2H), 4.44 (s, 4H), 3.98 (q, J=16.9 Hz, 2H), 2.40 (s, 3H); ¹³C NMR (101 MHz, CDCl₃) δ 162.11, 162.00, 161.44, 159.51, 131.16, 131.09, 131.06, 131.00, 129.86, 111.28, 111.08, 103.94, 103.69, 103.44, 70.50, 65.34, 21.06; ¹⁹F NMR (376 MHz, CDCl₃) δ −110.78, −114.37; ¹⁹F NMR (376 MHz, CDCl₃) δ −110.78, −114.37.

### 3-(2-(((4-Fluorobenzyl)oxy)methyl)-5-methylphenyl)-2-iminothiazolidin-4-one (C531)

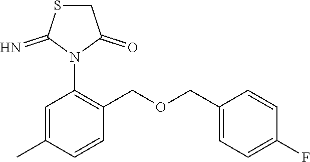 [SMILES: Cc1ccc(COCc2ccc(F)cc2)c(N2C(=N)SCC2=O)c1]

The title compound was prepared and was isolated as a yellow oil (111 mg, 93%): ¹H NMR (400 MHz, CDCl₃) δ 7.42 (d, J=7.8 Hz, 1H), 7.28 (dd, J=8.5, 5.5 Hz, 3H), 7.06-6.96 (m, 3H), 4.54-4.26 (m, 4H), 4.06-3.77 (m, 2H), 2.40 (s, 3H); ¹³C NMR (101 MHz, CDCl₃) δ 161.18, 158.73, 137.38, 131.31, 128.51, 127.89, 127.48, 127.26, 127.18, 112.94, 112.73, 69.10, 66.49, 18.66.

### 3-(2-(((4-Fluoro-2-(trifluoromethyl)benzyl)oxy)methyl)-5-methylphenyl)-2-iminothiazolidin-4-one (C532)

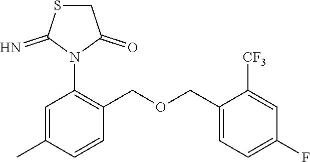 [SMILES: Cc1ccc(COCc2ccc(F)cc2C(F)(F)F)c(N2C(=N)SCC2=O)c1]

The title compound was prepared and was isolated as a yellow oil (66.3 mg, 85%): ¹H NMR (400 MHz, CDCl₃) δ 7.81 (s, 1H), 7.65 (dd, J=8.7, 5.6 Hz, 1H), 7.45 (d, J=7.6 Hz, 1H), 7.33 (dt, J=12.6, 6.3 Hz, 2H), 7.22 (d, J=8.7 Hz, 1H), 7.03 (s, 1H), 4.53 (d, J=51.4 Hz, 4H), 3.99 (q, J=16.9 Hz, 2H), 2.40 (s, 3H); ¹³C NMR (101 MHz, CDCl₃) δ 164.97, 162.93, 153.22, 139.61, 137.89, 132.18, 132.10, 131.94, 129.47, 124.36, 119.02, 118.81, 71.93, 67.53, 27.37, 21.50, 19.92.

### 2-Imino-3-(5-methyl-2-(((perfluorophenyl)methoxy)methyl)phenyl)thiazolidin-4-one (C533)

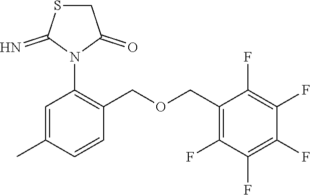 [SMILES: Cc1ccc(COCc2c(F)c(F)c(F)c(F)c2F)c(N2C(=N)SCC2=O)c1]

The title compound was prepared and was isolated as a yellow oil (58.1 mg, 87%): ¹H NMR (500 MHz, CDCl₃) δ 7.81 (s, 1H), 7.44 (d, J=7.8 Hz, 1H), 7.33-7.28 (m, 1H), 7.02 (s, 1H), 4.66-4.31 (m, 4H), 4.07 (s, 2H), 2.40 (s, 3H); ¹⁹F NMR (471 MHz, CDCl₃) δ −142.50, −142.52, −142.55, −142.57, −153.49, −161.77.

### 3-(2-(((2-Fluorobenzyl)oxy)methyl)-5-methylphenyl)-2-iminothiazolidin-4-one (C534)

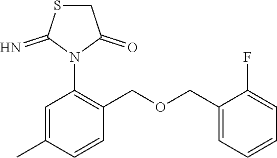 [SMILES: Cc1ccc(COCc2ccccc2F)c(N2C(=N)SCC2=O)c1]

The title compound was prepared and was isolated as a beige oil (65 mg, 78%): ¹H NMR (400 MHz, CDCl₃) δ 7.76 (s, 1H), 7.43 (d, J=7.8 Hz, 1H), 7.31 (qd, J=7.8, 2.3 Hz, 5H), 7.02 (s, 1H), 4.55-4.35 (m, 4H), 4.05-3.80 (m, 2H), 2.39 (s, 3H); ¹³C NMR (101 MHz, CDCl₃) δ 163.12, 160.62, 138.33, 137.96, 130.54, 129.83, 128.39, 127.88, 127.68, 72.66, 72.27, 68.88, 42.54, 21.05; ESIMS m/z 343 ([M−H]⁻).

### 3-(2-((Benzyloxy)methyl)-5-methylphenyl)-2-iminothiazolidin-4-one (C535)

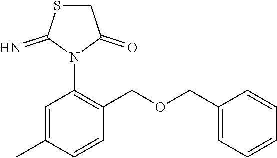 [SMILES: Cc1ccc(COCc2ccccc2)c(N2C(=N)SCC2=O)c1]

The title compound was prepared and was isolated as a beige oil (58 mg, 85%): ¹H NMR (400 MHz, CDCl₃) δ 7.76 (s, 1H), 7.43 (d, J=7.8 Hz, 1H), 7.31 (qd, J=7.8, 2.3 Hz, 6H), 7.02 (s, 1H), 4.56-4.36 (m, 4H), 3.96 (d, J=16.9 Hz, 1H), 3.84 (d, J=16.8 Hz, 1H), 2.39 (s, 3H); ¹³C NMR (101 MHz, CDCl₃) δ 163.71, 158.99, 130.85, 130.25, 124.67, 115.45, 113.73, 67.88, 66.33, 40.42, 22.56.

### 3-(2-(((3,5-Bis(trifluoromethyl)benzyl)oxy)methyl)-5-methylphenyl)-2-iminothiazolidin-4-one (C536)

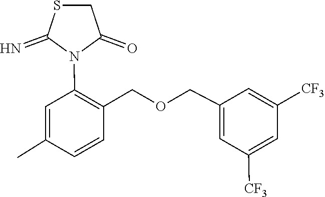 [SMILES: Cc1ccc(COCc2cc(C(F)(F)F)cc(C(F)(F)F)c2)c(N2C(=N)SCC2=O)c1]

The title compound was prepared and was isolated as a yellow oil (89 mg, 80%): ¹H NMR (500 MHz, CDCl₃) δ 7.77 (d, J=11.2 Hz, 3H), 7.40 (s, 1H), 7.30 (d, J=5.7 Hz, 1H), 7.05 (s, 1H), 4.50 (d, J=13.4 Hz, 4H), 4.04 (d, J=17.0 Hz, 1H), 3.94 (d, J=17.0 Hz, 1H), 2.41 (s, 3H); ¹⁹F NMR (471 MHz, CDCl₃) δ −62.96.

### 3-(2-(((2-Chlorobenzyl)oxy)methyl)-5-methylphenyl)-2-iminothiazolidin-4-one (C537)

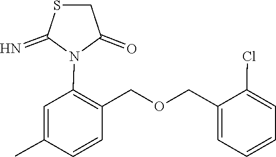 [SMILES: Cc1ccc(COCc2ccccc2Cl)c(N2C(=N)SCC2=O)c1]

The title compound was prepared and was isolated as a yellow oil (91 mg, 81%): ¹H NMR (500 MHz, CDCl₃) δ 7.80 (s, 1H), 7.46 (d, J=8.5 Hz, 2H), 7.36-7.28 (m, 2H), 7.26-7.18 (m, 2H), 7.04 (d, J=12.0 Hz, 1H), 4.52 (d, J=30.4 Hz, 4H), 3.97 (q, J=16.9 Hz, 2H), 2.40 (s, 3H).

### 2-Imino-3-(5-methyl-2-((thiophen-3-ylmethoxy)methyl)phenyl)thiazolidin-4-one (C538)

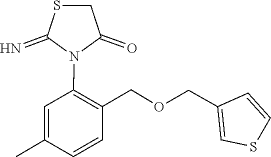 [SMILES: Cc1ccc(COCc2ccsc2)c(N2C(=N)SCC2=O)c1]

The title compound was prepared and was isolated as a yellow oil (90 mg, 80%): ¹H NMR (500 MHz, CDCl₃) δ 7.41 (d, J=7.8 Hz, 1H), 7.34-7.26 (m, 2H), 7.20 (dd, J=3.0, 1.1 Hz, 1H), 7.06 (dd, J=4.9, 1.3 Hz, 1H), 7.04-7.00 (m, 1H), 4.50-4.32 (m, 4H), 3.98 (d, J=16.9 Hz, 1H), 3.88 (d, J=16.9 Hz, 1H), 2.40 (s, 3H); ¹³C NMR (126 MHz, CDCl₃) δ 169.31, 160.88, 138.28, 129.85, 127.46, 125.94, 123.14, 68.33, 67.33, 21.05.

### 3-(2-((Cyclopropylmethoxy)methyl)-5-methylphenyl)-2-iminothiazolidin-4-one (C539)

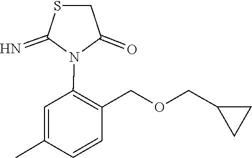 [SMILES: Cc1ccc(COCC2CC2)c(N2C(=N)SCC2=O)c1]

The title compound was prepared and was isolated as a yellow oil (40.5 mg, 71%): ¹H NMR (500 MHz, CDCl₃) δ 7.80 (s, 1H), 7.39 (d, J=7.4 Hz, 1H), 7.27 (d, J=14.0 Hz, 1H), 7.01 (s, 1H), 4.50 (d, J=13.0 Hz, 1H), 4.37 (d, J=13.1 Hz, 1H), 4.09 (q, J=15.9, 15.3 Hz, 2H), 3.18 (d, J=7.2 Hz, 2H), 2.38 (s, 3H), 1.12 (m, 1H), 0.50 (s, 2H), 0.17 (d, J=5.4 Hz, 2H); ¹³C NMR (126 MHz, CDCl₃) δ 172.14, 163.64, 133.14, 131.35, 128.51, 122.17, 105.25, 73.40, 68.85, 38.28, 21.03, 10.48, 2.96, 2.85.

### 3-(2-((Benzo[d]thiazol-2-ylmethoxy)methyl)-5-methylphenyl)-2-iminothiazolidin-4-one (C540)

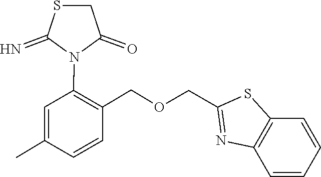 [SMILES: Cc1ccc(COCc2nc3ccccc3s2)c(N2C(=N)SCC2=O)c1]

The title compound was prepared and was isolated as a yellow oil (61.5 mg, 55%): 1H NMR (500 MHz, CDCl₃) δ 8.03-7.96 (m, 1H), 7.93-7.81 (m, 1H), 7.48 (ddt, J=8.5, 7.1, 1.3 Hz, 2H), 7.39 (ddd, J=8.2, 7.2, 1.2 Hz, 1H), 7.34-7.28 (m, 1H), 7.09-6.95 (m, 1H), 4.82 (s, 2H), 4.73-4.50 (m, 2H), 4.11-3.96 (m, 2H), 2.41 (s. 3H); ¹³C NMR (126 MHz, CDCl₃) δ 172.36, 169.30, 153.03, 135.03, 129.91, 126.28, 126.07, 125.12, 123.02, 121.84, 121.79, 70.11, 69.44, 39.71.

### 2-Imino-3-(5-methyl-2-(((1-methyl-3-(trifluoromethyl)-1H-thieno[2,3-c]pyrazol-5-yl)methoxy)methyl)phenyl)thiazolidin-4-one (C541)

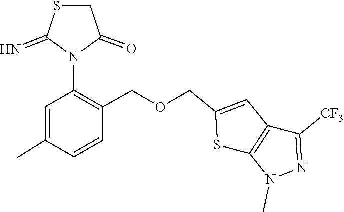 [SMILES: Cc1ccc(COCc2cc3c(C(F)(F)F)nn(C)c3s2)c(N2C(=N)SCC2=O)c1]

The title compound was prepared and was isolated as a yellow oil (103 mg, 52%): 1H NMR (500 MHz, CDCl₃) δ 7.42 (d, J=7.8 Hz, 1H), 7.30 (d, J=7.8 Hz, 1H), 7.03 (s, 1H), 6.90-6.85 (m, 1H), 4.58-4.35 (m, 4H), 4.01 (m, 5H), 2.40 (s, 3H); ¹⁹F NMR (471 MHz, CDCl₃) δ −61.09; ESIMS m/z 455 ([M+H]⁺).

### 3-(2-((Benzo[d]oxazol-2-ylmethoxy)methyl)-5-methylphenyl)-2-iminothiazolidin-4-one (C542)

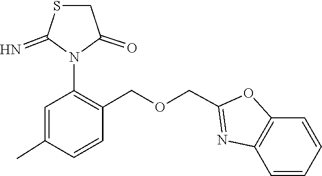 [SMILES: Cc1ccc(COCc2nc3ccccc3o2)c(N2C(=N)SCC2=O)c1]

The title compound was prepared and was isolated as a yellow oil (45 mg, 27%): 1H NMR (400 MHz, CDCl₃) δ 7.73 (ddd, J=7.4, 5.2, 2.9 Hz, 1H), 7.57-7.43 (m, 2H), 7.41-7.26 (m, 4H), 7.06-6.90 (m, 1H), 4.63 (qd, J=7.9, 3.7 Hz, 4H), 4.42-3.95 (m, 2H), 2.39 (s, 3H); ESIMS m/z 368 ([M+H]⁺). 3-(2-((Benzyloxy)methyl)-5-chlorophenyl)-2-iminothiazolidin-4-one (C543)

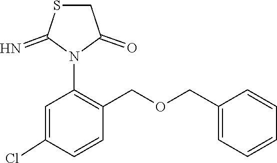 [SMILES: N=C1SCC(=O)N1c1cc(Cl)ccc1COCc1ccccc1]

The title compound was prepared and was isolated as a purple solid (126 mg, 53%): ¹H NMR (500 MHz, CDCl₃) δ 7.80 (s, 1H), 7.46 (d, J=8.5 Hz, 2H), 7.36-7.28 (m, 2H), 7.26-7.18 (m, 2H), 7.04 (d, J=12.0 Hz, 1H), 4.52 (d, J=30.4 Hz, 4H), 3.97 (q, J=16.9 Hz, 2H); ¹³C NMR (101 MHz, CDCl₃) δ 164.14, 160.28, 141.75, 134.24, 130.86, 130.24, 129.56, 128.36, 127.76, 72.11, 67.82.

### 3-(2-((Benzyloxy)methyl)-4-chlorophenyl)-2-iminothiazolidin-4-one (C544)

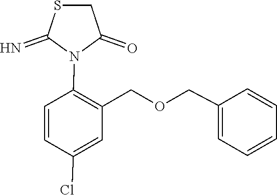 [SMILES: N=C1SCC(=O)N1c1ccc(Cl)cc1COCc1ccccc1]

The title compound was prepared and was isolated as a yellow oil (73 mg, 65%): ¹H NMR (500 MHz, CDCl₃) δ 7.81 (s, 1H), 7.58 (s, 1H), 7.43 (dd, J=8.4, 2.4 Hz, 1H), 7.39-7.28 (m, 5H), 7.14 (d, J=8.4 Hz, 1H), 4.47 (s, 4H), 3.98 (d, J=16.9 Hz, 1H), 3.86 (d, J=16.9 Hz, 1H); ¹³C NMR (126 MHz, CDCl₃) δ 171.05, 169.56, 137.57, 130.63, 129.91, 129.27, 128.49, 127.88, 72.65, 68.34, 34.20.

### 3-(2-((Benzyloxy)methyl)-5-bromophenyl)-2-iminothiazolidin-4-one (C545)

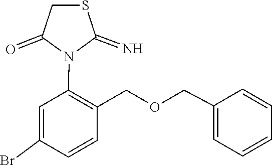 [SMILES: N=C1SCC(=O)N1c1cc(Br)ccc1COCc1ccccc1]

The title compound was prepared and was isolated as a yellow oil (117 mg, 66%): ¹H NMR (400 MHz, CDCl₃) δ 7.82 (s, 1H), 7.60 (d, J=7.2 Hz, 1H), 7.43 (d, J=7.5 Hz, 1H), 7.40-7.28 (m, 6H), 4.43 (d, J=10.3 Hz, 4H), 4.04-3.73 (m, 2H); ¹³C NMR (101 MHz, CDCl₃) δ 170.68, 161.10, 137.75, 136.14, 134.17, 132.91, 132.42, 131.11, 128.45, 127.85, 121.74, 76.72, 72.43, 68.52, 34.35.

### 3-(2-((Benzyloxy)methyl)-5-(trifluoromethyl)phenyl)-2-iminothiazolidin-4-one (C546)

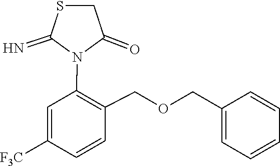 [SMILES: N=C1SCC(=O)N1c1cc(C(F)(F)F)ccc1COCc1ccccc1]

The title compound was prepared and was isolated as a yellow oil (94 mg, 56%): ¹H NMR (400 MHz, CDCl₃) δ 7.81 (d, J=29.5 Hz, 1H), 7.72 (s, 2H), 7.48 (s, 1H), 7.41-7.27 (m, 5H), 4.67-4.31 (m, 4H), 4.06-3.76 (m, 2H); ¹³C NMR (101 MHz, CDCl₃) δ 170.49, 158.93, 141.22, 138.92, 134.41, 129.80, 128.61, 128.03, 127.95, 126.81, 126.73, 124.37, 121.74, 73.85, 68.56, 40.52; ¹⁹F NMR (376 MHz, CDCl₃) δ −62.51.

### 3-(2-((Benzyloxy)methyl)phenyl)-2-iminothiazolidin-4-one (C547)

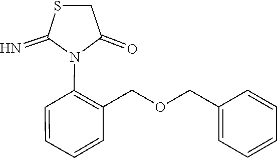 [SMILES: N=C1SCC(=O)N1c1ccccc1COCc1ccccc1]

The title compound was prepared and was isolated as a yellow oil (112 mg, 66%): ¹H NMR (500 MHz, CDCl₃) δ 7.79 (s, 1H), 7.56 (s, 1H), 7.48 (s, 2H), 7.40-7.27 (m, 4H), 7.17 (dd, J=36.2, 6.4 Hz, 1H), 4.67-4.29 (m, 4H), 4.26-3.77 (m, 2H); ¹³C NMR (126 MHz, CDCl₃) δ 172.91, 137.39, 129.38, 128.42, 128.11, 127.93, 127.88, 127.74, 73.26, 72.47, 68.51.

### 2-Imino-3-(2-methyl-5-(trifluoromethyl)phenyl)thiazolidin-4-one (C548)

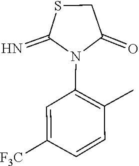 [SMILES: Cc1ccc(C(F)(F)F)cc1N1C(=N)SCC1=O]

The title compound was prepared and was isolated as a yellow oil (188 mg, 41%): ¹H NMR (400 MHz, CDCl₃) δ 7.71 (d, J=8.1 Hz, 1H), 7.43 (d, J=8.0 Hz, 1H), 7.17 (s, 1H), 4.23-3.98 (m, 2H), 2.46 (s, 3H); ¹³C NMR (101 MHz, CDCl₃) δ 170.47, 161.89, 144.56, 131.65, 131.08, 127.75, 127.71, 127.66, 125.93, 125.61, 124.14, 121.42, 33.99, 21.10.

### 2-Imino-3-(4-methyl-[1,1′-biphenyl]-2-yl)thiazolidin-4-one (C549)

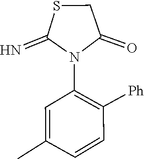 [SMILES: Cc1ccc(-c2ccccc2)c(N2C(=N)SCC2=O)c1]

The title compound was prepared and was isolated as a yellow oil (206 mg, 45%): ¹H NMR (400 MHz, CDCl₃) δ 7.40-7.30 (m, 6H), 7.27 (dd, J=5.9, 1.7 Hz, 2H), 7.07 (d, J=10.9 Hz, 1H), 3.94-3.54 (m, 2H), 2.43 (d, J=3.3 Hz, 3H); ¹³C NMR (101 MHz, CDCl₃) δ 172.71, 170.80, 162.60, 139.43, 139.33, 139.09, 138.54, 138.04, 131.48, 131.41, 131.32, 131.17, 130.85, 129.53, 129.26, 128.57, 128.45, 128.39, 128.15, 128.09, 127.84, 127.73, 73.19, 72.77, 27.33, 27.08, 25.21, 24.46.

### 3-([1,1′-Biphenyl]-2-yl)-2-iminothiazolidin-4-one (C550)

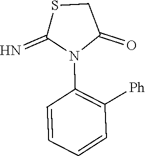 [SMILES: N=C1SCC(=O)N1c1ccccc1-c1ccccc1]

The title compound was prepared and was isolated as a yellow oil (78 mg, 43%): ¹H NMR (500 MHz, CDCl₃) δ 7.56-7.49 (m, 3H), 7.35 (dddd, J=14.1, 11.6, 5.6, 2.3 Hz, 4H), 7.30-7.27 (m, 2H), 3.86 (d, J=16.9 Hz, 1H), 3.66 (d, J=16.9 Hz, 1H); ESIMS m/z 269 ([M+H]⁺).

### 3-([1,1′-Biphenyl-3-yl)-2-iminothiazolidin-4-one (C551)

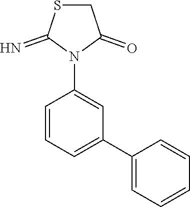 [SMILES: N=C1SCC(=O)N1c1cccc(-c2ccccc2)c1]

The title compound was prepared and was isolated as a yellow oil (104 mg, 43%): ¹H NMR (500 MHz, CDCl₃) δ 7.80 (s, 1H), 7.59-7.45 (m, 3H), 7.37 (q, J=8.6 Hz, 3H), 7.28 (d, J=6.6 Hz, 3H), 3.86 (d, J=16.9 Hz, 1H), 3.66 (d, J=16.9 Hz, 1H); ESIMS m/z 269 ([M+H]⁺).

### 3-([1,1′-Biphenyl]-4-yl)-2-iminothiazolidin-4-one (C552)

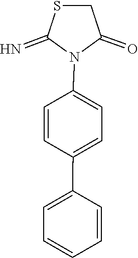 [SMILES: N=C1SCC(=O)N1c1ccc(-c2ccccc2)cc1]

The title compound was prepared and was isolated as a yellow solid (88 mg, 51%): ¹H NMR (500 MHz, CDCl₃) δ 7.94 (s, 1H), 7.72 (d, J=7.7 Hz, 2H), 7.58 (d, J=7.6 Hz, 2H), 7.45 (t, J=7.5 Hz, 2H), 7.40-7.30 (m, 3H), 4.10 (s, 2H); ¹³C NMR (126 MHz, CDCl₃) δ 171.18, 140.13, 128.89, 128.80, 128.38, 127.83, 127.61, 127.34, 126.88, 120.32, 73.45.

### 3-(4-Fluoro-2,5-dimethylphenyl)-2-iminothiazolidin-4-one (C553)

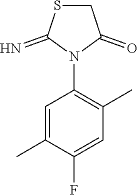 [SMILES: Cc1cc(N2C(=N)SCC2=O)c(C)cc1F]

The title compound was prepared and was isolated as a yellow oil (76 mg, 41%): ¹H NMR (500 MHz, CDCl₃) δ 7.87 (s, 1H), 7.07-6.81 (m, 2H), 4.42-4.04 (m, 2H), 2.26 (s, 3H), 2.12 (d, J=3.0 Hz, 3H); ¹³C NMR (126 MHz, CDCl₃) δ 162.58, 160.59, 137.42, 131.42, 127.24, 124.35, 117.89, 117.70, 73.40, 73.13, 27.65, 27.46, 25.60, 25.07.

### 2-Imino-3-(2,3,5-trimethylphenyl)thiazolidin-4-one (C554)

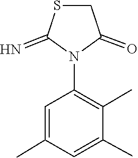 [SMILES: Cc1cc(C)c(C)c(N2C(=N)SCC2=O)c1]

The title compound was prepared and was isolated as a yellow oil (303 mg, 65%): ¹H NMR (400 MHz, CDCl₃) δ 7.09 (s, 1H), 6.81-6.68 (m, 1H), 4.38 (m, 1H), 4.08 (d, J=1.3 Hz, 2H), 2.30 (d, J=5.2 Hz, 9H); ¹³C NMR (101 MHz, CDCl₃) δ 170.86, 160.79, 138.59, 136.70, 132.78, 132.69, 132.22, 131.68, 126.42, 126.35, 126.11, 77.36, 77.05, 76.73, 73.45, 73.17, 58.82, 58.54, 27.72, 27.51, 25.55, 24.92.

### 2-Imino-3-(2-methoxy-3-methylphenyl)thiazolidin-4-one (C555)

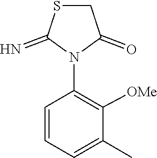 [SMILES: COc1c(C)cccc1N1C(=N)SCC1=O]

The title compound was prepared and was isolated as a yellow oil (349 mg, 71%): ¹H NMR (400 MHz, CDCl₃) δ 7.33 (d, J=7.0 Hz, 1H), 7.18 (t, J=7.7 Hz, 1H), 7.08-7.01 (m, 1H), 4.12 (d, J=3.9 Hz, 2H), 3.74 (s, 3H), 2.37 (s, 3H); ¹³C NMR (101 MHz, CDCl₃) δ 170.98, 161.43, 154.90, 133.43, 133.14, 127.38, 126.79, 124.83, 60.95, 34.01, 16.31.

### 2-Imino-3-(5-methyl-2-(((4-(trifluoromethoxy)benzyl)oxy)methyl)phenyl)thiazolidin-4-one (C556)

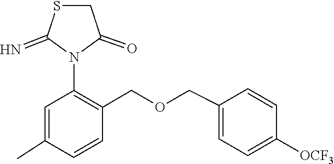 [SMILES: Cc1ccc(COCc2ccc(OC(F)(F)F)cc2)c(N2C(=N)SCC2=O)c1]

The title compound was prepared and was isolated as a yellow oil (92 mg, 83%): ¹H NMR (500 MHz, CDCl₃) δ 7.54-7.44 (m, 2H), 7.30 (tt, J=7.5, 4.1 Hz, 3H), 7.24-7.21 (m, 1H), 7.02 (s, 1H), 4.63-4.40 (m, 4H), 4.01 (d, J=16.9 Hz, 1H), 3.93 (d, J=16.9 Hz, 1H), 2.40 (s, 3H); ¹³C NMR (126 MHz, CDCl₃) δ 171.00, 160.60, 146.79, 139.76, 133.03, 130.97, 130.88, 130.10, 129.78, 129.49, 128.82, 126.91, 121.59, 120.31, 119.54, 69.39, 66.58, 34.03, 21.07; ¹⁹F NMR (471 MHz, CDCl₃) δ −57.17.

### Example 128: Preparation of 2-chloro-N-(5-methyl-2-(((4-(trifluoromethoxy)benzyl)oxy)methyl)phenyl)acetamide (C557)

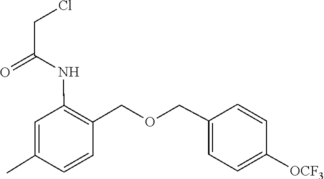 [SMILES: Cc1ccc(COCc2ccc(OC(F)(F)F)cc2)c(NC(=O)CCl)c1]

5-Methyl-2-(((4-(trifluoromethoxy)benzyl)oxy)methyl)aniline (KE-3ad; 250 mg, 0.803 mmol) and sodium bicarbonate (169 mg, 2.01 mmol) were added to a dried 10-mL vial equipped with a Teflon-coated stirbar and capped with a rubber septum. The vial was evacuated and backfilled with nitrogen three times. Degassed and dry acetone (6 mL) was added by syringe, followed by 2-chloroacetyl chloride (77 μL, 0.96 mmol). The resulting mixture was stirred at 23° C. for 1.5 h. The reaction mixture was filtered over diatomaceous earth and concentrated in vacuo. The resulting residue was diluted with saturated sodium bicarbonate (50 mL) and DCM (50 mL). The phases were separated, and the organic layer was collected. The organic layer was dried with magnesium sulfate and concentrated in vacuo to afford a white powder residue. Purification of the residue by flash chromatography eluting with 0-60% EtOAc-hexanes provided the title compound as a white solid (295 mg, 90%): ¹H NMR (400 MHz, CDCl₃) δ 9.58 (s, 1H), 8.01-7.96 (m, 1H), 7.40-7.32 (m, 2H), 7.23-7.16 (m, 2H), 7.08 (d, J=7.6 Hz, 1H), 6.94 (dt, J=7.7, 1.3 Hz, 1H), 4.58 (d, J=13.0 Hz, 4H), 4.17 (s, 2H), 2.37 (s, 3H); ¹³C NMR (101 MHz, CDCl₃) δ 164.30, 139.65, 136.66, 135.99, 129.48, 129.24, 125.45, 123.89, 122.63, 120.99, 71.22, 71.17, 43.09, 21.50; ¹⁹F NMR (376 MHz, CDCl₃) δ −57.76.

The following compounds were prepared according to the procedure described in Example 128:

### 2-Chloro-N-(2-(((3-fluorobenzyl)oxy)methyl)-5-methylphenyl)acetamide (C558)

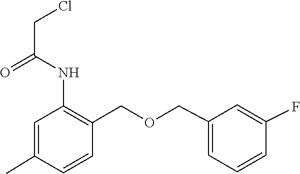 [SMILES: Cc1ccc(COCc2cccc(F)c2)c(NC(=O)CCl)c1]

The title compound was prepared and was isolated as a white solid (103 mg, 97%): ¹H NMR (400 MHz, CDCl₃) δ 9.55 (s, 1H), 8.00-7.95 (m, 1H), 7.44-7.24 (m, 2H), 7.17-7.00 (m, 3H), 6.92 (ddd, J=7.6, 1.7, 0.8 Hz, 1H), 4.65-4.54 (m, 4H), 4.14 (s, 2H), 2.36 (s, 3H); ¹³C NMR (101 MHz, CDCl₃) δ 164.21, 161.86, 159.41, 139.36, 136.54, 130.14, 130.09, 129.69, 129.60, 129.33, 125.23, 124.28, 124.14, 123.99, 123.96, 123.82, 122.45, 115.27, 115.06, 71.22, 65.63, 65.59, 42.89, 21.31.

### 2-Chloro-N-(5-methyl-2-(((2-(trifluoromethoxy)benzyl)oxy)methyl)phenyl)acetamide (C559)

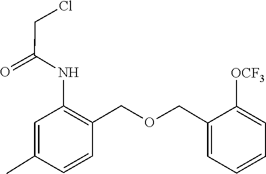 [SMILES: Cc1ccc(COCc2ccccc2OC(F)(F)F)c(NC(=O)CCl)c1]

The title compound was prepared and was isolated as a white solid (311 mg, 95%): ¹H NMR (400 MHz, CDCl₃) δ 9.57 (s, 1H), 8.01-7.96 (m, 1H), 7.52 (dd, J=7.4, 2.0 Hz, 1H), 7.39-7.28 (m, 2H), 7.28-7.21 (m, 1H), 7.11 (d, J=7.6 Hz, 1H), 6.98-6.91 (m, 1H), 4.64 (d, J=7.5 Hz, 4H), 4.16 (s, 2H), 2.38 (s, 3H); ¹³C NMR (101 MHz, CDCl₃) δ 164.32, 146.92, 139.61, 136.66, 130.16, 129.68, 129.48, 129.18, 126.90, 125.46, 123.98, 122.64, 121.81, 120.41, 119.25, 71.57, 66.30, 43.04, 21.50; ¹⁹F NMR (376 MHz, CDCl₃) δ −57.26.

### 2-Chloro-N-(2-(((2,4-difluorobenzyl)oxy)methyl)-5-methylphenyl)acetamide (C560)

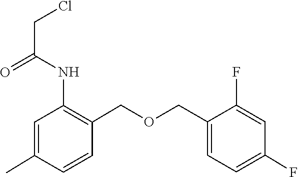 [SMILES: Cc1ccc(COCc2ccc(F)cc2F)c(NC(=O)CCl)c1]

The title compound was prepared and was isolated as a white solid (545 mg, 90%): ¹H NMR (500 MHz, CDCl₃) δ 9.53 (s, 1H), 7.99 (d, J=1.7 Hz, 1H), 7.09 (d, J=7.7 Hz, 1H), 6.95 (dd, J=7.6, 1.8 Hz, 1H), 6.87 (h, J=4.5 Hz, 2H), 6.74 (tt, J=9.0, 2.4 Hz, 1H), 4.61 (s, 2H), 4.54 (s, 2H), 4.19 (s, 2H), 2.38 (s, 3H); ¹³C NMR (101 MHz, CDCl₃) δ 163.63, 147.58, 130.59, 130.49, 126.95, 124.52, 117.76, 60.41, 43.20, 15.88.

### 2-Chloro-N-(2-(((4-fluorobenzyl)oxy)methyl)-5-methylphenyl)acetamide (C561)

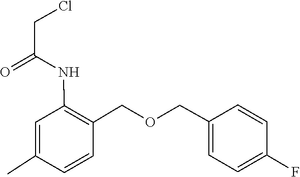 [SMILES: Cc1ccc(COCc2ccc(F)cc2)c(NC(=O)CCl)c1]

The title compound was prepared and was isolated as a white solid (311 mg, 90%): ¹H NMR (500 MHz, CDCl₃) δ 9.59 (s, 1H), 8.01-7.97 (m, 1H), 7.51-7.46 (m, 1H), 7.37 (dd, J=7.4, 1.8 Hz, 1H), 7.31-7.21 (m, 2H), 7.13 (d, J=7.7 Hz, 1H), 6.95 (dt, J=7.5, 1.4 Hz, 1H), 4.68 (d, J=1.2 Hz, 4H), 4.17 (s, 2H), 2.38 (s, 3H); ¹³C NMR (126 MHz, CDCl₃) δ 164.23, 139.46, 136.55, 134.95, 132.83, 129.39, 129.22, 129.01, 128.87, 126.71, 125.33, 123.89, 122.50, 71.51, 69.10, 42.97, 21.38; ¹⁹F NMR (471 MHz, CDCl₃) δ −62.74.

### 2-Chloro-N-(2-(((4-fluoro-2-(trifluoromethyl)benzyl)oxy)methyl)-5-methylphenyl)acetamide (C562)

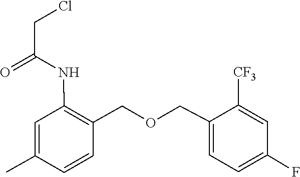 [SMILES: Cc1ccc(COCc2ccc(F)cc2C(F)(F)F)c(NC(=O)CCl)c1]

The title compound was prepared and was isolated as a white solid (295 mg, 90%): ¹H NMR (500 MHz, CDCl₃) δ 9.50 (s, 1H), 7.99 (d, J=1.7 Hz, 1H), 7.66 (dd, J=8.7, 5.4 Hz, 1H), 7.36 (dd, J=9.0, 2.7 Hz, 1H), 7.24 (dt, J=8.3, 4.2 Hz, 1H), 7.11 (d, J=7.6 Hz, 1H), 6.96 (dd, J=7.2, 1.6 Hz, 1H), 4.72 (d, J=1.7 Hz, 2H), 4. 64 (s, 2H), 4.17 (s, 2H), 2.38 (s, 3H); ¹³C NMR (126 MHz, CDCl₃) δ 164.27, 162.49, 160.51, 139.79, 136.57, 131.93, 131.21, 131.14, 129.52, 129.28, 129.22, 125.58, 123.82, 122.70, 122.24, 118.92, 118.75, 113.70, 113.66, 113.50, 113.45, 77.30, 77.04, 76.79, 71.66, 67.59, 67.57, 43.07, 21.53; ¹⁹F NMR (471 MHz, CDCl₃) δ −60.52, −112.74.

### 2-Chloro-N-(5-methyl-2-(((perfluorophenyl)methoxy)methyl)phenyl)acetamide (C563)

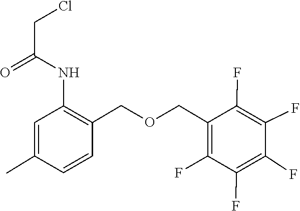 [SMILES: Cc1ccc(COCc2c(F)c(F)c(F)c(F)c2F)c(NC(=O)CCl)c1]

The title compound was prepared and was isolated as a white solid (256 mg, 98%): ¹H NMR (500 MHz, CDCl₃) δ 9.35 (s, 1H), 7.96 (d, J=1.7 Hz, 1H), 7.12 (d, J=7.7 Hz, 1H), 6.95 (dd, J=7.8, 1.7 Hz, 1H), 4.63 (d, J=2.1 Hz, 4H), 4.18 (s, 2H), 2.37 (s, 3H); ¹³C NMR (126 MHz, CDCl₃) δ 164.34, 146.56, 144.60, 142.53, 139.96, 138.44, 136.49, 129.64, 125.55, 123.35, 122.79, 110.63, 110.46, 71.81, 58.72, 43.02, 21.48.

### 2-Chloro-N-(2-(((2-fluorobenzyl)oxy)methyl)-5-methylphenyl)acetamide (C564)

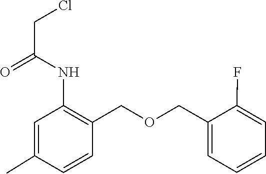 [SMILES: Cc1ccc(COCc2ccccc2F)c(NC(=O)CCl)c1]

The title compound was prepared and was isolated as a white solid (104 mg, 94%): ¹H NMR (400 MHz, CDCl₃) δ 9.56 (s, 1H), 8.01-7.96 (m, 1H), 7.45-7.25 (m, 2H), 7.18-7.01 (m, 3H), 6.93 (ddd, J=7.6, 1.7, 0.8 Hz, 1H), 4.66-4.56 (m, 4H), 4.16 (s, 2H), 2.37 (s, 3H); ¹³C NMR (101 MHz, CDCl₃) δ 164.39, 162.05, 159.59, 139.54, 136.72, 130.32, 130.28, 129.87, 129.79, 129.51, 125.41, 124.46, 124.32, 124.18, 124.14, 124.00, 122.64, 115.45, 115.24, 77.34, 77.02, 76.71, 71.40, 65.81, 65.77, 43.08, 21.49; ESIMS m/z 320 ([M−H]⁻).

### N-(2-((Benzyloxy)methyl)-5-methylphenyl)-2-chloroacetamide (C565)

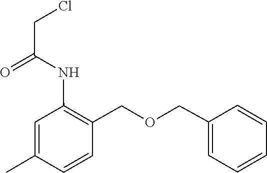 [SMILES: Cc1ccc(COCc2ccccc2)c(NC(=O)CCl)c1]

The title compound was prepared and was isolated as a white solid (128 mg, 91%): ¹H NMR (400 MHz, CDCl₃) δ 9.65 (s, 1H), 7.99 (d, J=1.6 Hz, 1H), 7.41-7.27 (m, 5H), 7.07 (d, J=7.6 Hz, 1H), 6.96-6.89 (m, 1H), 4.58 (d, J=3.4 Hz, 4H), 4.15 (s, 2H), 2.37 (s, 3H); ¹³C NMR (101 MHz, CDCl₃) δ 164.38, 139.45, 137.19, 136.75, 129.45, 128.49, 128.01, 127.98, 125.37, 124.12, 122.55, 72.19, 70.97, 43.09, 21.50; ESIMS m/z 302 ([M−H]⁻).

### N-(2-(((3,5-Bis(trifluoromethyl)benzyl)oxy)methyl)-5-methylphenyl)-2-chloroacetamide (C566)

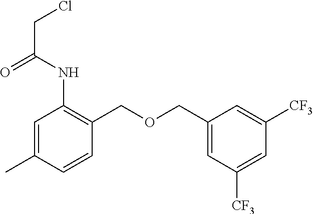 [SMILES: Cc1ccc(COCc2cc(C(F)(F)F)cc(C(F)(F)F)c2)c(NC(=O)CCl)c1]

The title compound was prepared and was isolated as a white solid (287 mg, 90%): ¹H NMR (500 MHz, CDCl₃) δ 9.48 (s, 1H), 7.99 (s, 1H), 7.80 (d, J=11.8 Hz, 3H), 7.11 (d, J=7.6 Hz, 1H), 6.97 (d, J=7.6 Hz, 1H), 4.67 (d, J=11.8 Hz, 4H), 4.18 (s, 2H), 2.38 (s, 3H); ¹³C NMR (126 MHz, CDCl₃) δ 164.22, 140.06, 139.97, 136.56, 132.14, 131.88, 131.61, 131.35, 129.60, 127.42, 126.47, 125.59, 124.30, 123.34, 122.72, 122.13, 121.76, 121.73, 121.70, 119.96, 71.75, 70.42, 43.05, 21.50.

### 2-Chloro-N-(2-(((2-chlorobenzyl)oxy)methyl)-5-methylphenyl)acetamide (C567)

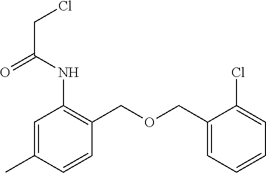 [SMILES: Cc1ccc(COCc2ccccc2Cl)c(NC(=O)CCl)c1]

The title compound was prepared and was isolated as a white solid (326 mg, 96%): ¹H NMR (500 MHz, CDCl₃) δ 9.58 (s, 1H), 8.01-7.97 (m, 1H), 7.50-7.45 (m, 1H), 7.36 (dd, J=7.4, 1.8 Hz, 1H), 7.30-7.21 (m, 2H), 7.12 (d, J=7.7 Hz, 1H), 6.95 (dt, J=7.5, 1.4 Hz, 1H), 4.67 (d, J=1.2 Hz, 4H), 4.16 (s, 2H), 2.38 (s, 3H); ¹³C NMR (126 MHz, CDCl₃) δ 164.38, 139.61, 136.69, 135.10, 132.98, 129.54, 129.37, 129.16, 129.02, 126.86, 125.48, 124.04, 122.65, 77.31, 77.05, 76.80, 71.66, 69.25, 43.12, 21.53.

### 2-Chloro-N-(5-methyl-2-((thiophen-3-ylmethoxy)methyl)phenyl)acetamide (C568)

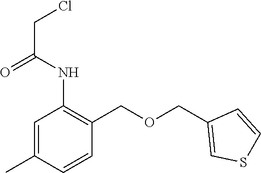 [SMILES: Cc1ccc(COCc2ccsc2)c(NC(=O)CCl)c1]

The title compound was prepared and was isolated as a white solid (280 mg, 95%): ¹H NMR (500 MHz, CDCl₃) δ 9.64 (s, 1H), 7.99 (d, J=1.7 Hz, 1H), 7.32 (dd, J=5.0, 3.0 Hz, 1H), 7.23 (dd, J=3.0, 1.2 Hz, 1H), 7.14-6.95 (m, 2H), 6.95-6.89 (m, 1H), 4.58 (d, J=6.7 Hz, 4H), 4.16 (s, 2H), 2.37 (s, 3H); ¹³C NMR (126 MHz, CDCl₃) δ 164.33, 139.45, 138.23, 136.71, 129.44, 128.01, 127.38, 126.20, 125.85, 125.36, 123.99, 123.49, 122.85, 122.49, 121.49, 70.83, 67.21, 43.09, 21.49.

### 2-Chloro-N-(2-((cyclopropylmethoxy)methyl)-5-methylphenyl)acetamide (C569)

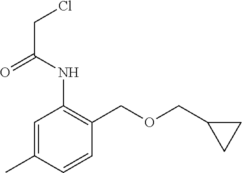 [SMILES: Cc1ccc(COCC2CC2)c(NC(=O)CCl)c1]

The title compound was prepared and was isolated as a white solid (286 mg, 97%): ¹H NMR (500 MHz, CDCl₃) δ 9.75 (s, 1H), 7.99 (d, J=1.5 Hz, 1H), 7.08 (d, J=7.6 Hz, 1H), 6.91 (dd, J=7.7, 1.5 Hz, 1H), 4.56 (s, 2H), 4.20 (s, 2H), 3.35 (d, J=7.0 Hz, 2H), 2.36 (s, 3H), 1.16-1.04 (m, 1H), 0.60-0.50 (m, 2H), 0.20 (dt, J=6.0, 4.5 Hz, 2H); ¹³C NMR (126 MHz, CDCl₃) δ 164.36, 139.24, 136.70, 129.25, 125.27, 124.32, 122.40, 75.11, 71.45, 43.09, 21.48, 10.45, 3.18.

### N-(2-((Benzo[d]thiazol-2-ylmethoxy)methyl)-5-methylphenyl)-2-chloroacetamide (C570)

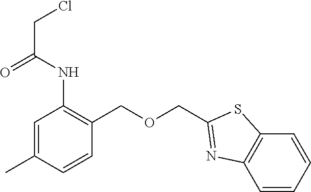 [SMILES: Cc1ccc(COCc2nc3ccccc3s2)c(NC(=O)CCl)c1]

The title compound was prepared and was isolated as a white solid (92 mg, 69%): ¹H NMR (400 MHz, CDCl₃) δ 9.44 (s, 1H), 8.02 (dd, J=8.2, 1.0 Hz, 1H), 7.99-7.94 (m, 1H), 7.94-7.87 (m, 1H), 7.50 (ddd, J=8.2, 7.2, 1.3 Hz, 1H), 7.41 (td, J=7.6, 1.2 Hz, 1H), 7.13 (d, J=7.6 Hz, 1H), 7.00-6.93 (m, 1H), 4.97 (s, 2H), 4.75 (s, 2H), 4.21 (s, 2H), 2.38 (s, 3H); ¹³C NMR (101 MHz, CDCl₃) δ 168.71, 164.47, 152.81, 140.00, 136.53, 134.94, 129.77, 126.29, 125.70, 125.39, 123.52, 123.07, 123.00, 121.85, 71.89, 69.22, 43.15, 21.49.

### 2-Chloro-N-(5-methyl-2-(((1-methyl-3-(trifluoromethyl)-1H-thieno[2,3-c]pyrazol-5-yl)methoxy)methyl)phenyl)acetamide (C571)

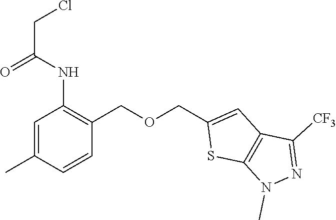 [SMILES: Cc1ccc(COCc2cc3c(C(F)(F)F)nn(C)c3s2)c(NC(=O)CCl)c1]

The title compound was prepared and was isolated as a white solid (251 mg, 98%): ¹H NMR (500 MHz, CDCl₃) δ 9.47 (s, 1H), 7.98 (s, 1H), 7.08 (d, J=7.7 Hz, 1H), 6.95 (d, J=8.7 Hz, 2H), 4.70 (d, J=0.7 Hz, 2H), 4.61 (s, 2H), 4.19 (s, 2H), 4.03 (s, 3H), 2.38 (s, 3H); ¹³C NMR (126 MHz, CDCl₃) δ 164.46, 146.03, 140.84, 139.98, 136.76, 132.97, 132.66, 129.75, 126.43, 125.71, 123.68, 122.90, 122.39, 115.11, 70.72, 67.48, 43.28, 39.03, 21.63.

### N-(2-((Benzo[d]oxazol-2-ylmethoxy)methyl)-5-methylphenyl)-2-chloroacetamide (C572)

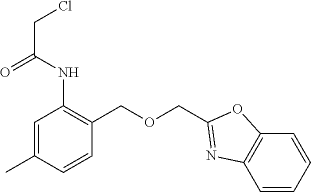 [SMILES: Cc1ccc(COCc2nc3ccccc3o2)c(NC(=O)CCl)c1]

The title compound was prepared and was isolated as a white solid (33.1 mg, 49%): ¹H NMR (500 MHz, CDCl₃) δ 9.47 (s, 1H), 7.98 (s, 1H), 7.75 (dd, J=5.6, 3.4 Hz, 1H), 7.57-7.51 (m, 1H), 7.42-7.33 (m, 2H), 7.13 (d, J=7.6 Hz, 1H), 6.93 (d, J=7.6 Hz, 1H), 4.83 (s, 2H), 4.75 (s, 2H), 4.25 (s, 2H), 2.36 (s, 3H); ¹³C NMR (126 MHz, CDCl₃) δ 164.62, 162.11, 150.90, 140.65, 139.99, 136.70, 129.89, 125.65, 125.55, 124.74, 123.26, 122.98, 120.28, 110.86, 71.92, 64.27, 43.21, 21.49.

### N-(2-((Benzyloxy)methyl)-5-chlorophenyl)-2-chloroacetamide (C573)

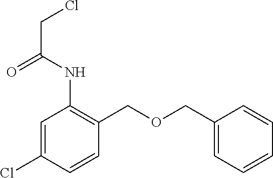 [SMILES: O=C(CCl)Nc1cc(Cl)ccc1COCc1ccccc1]

The title compound was prepared and was isolated as a clear colorless oil (228 mg, 92%): ¹H NMR (500 MHz, CDCl₃) δ 9.77 (s, 1H), 8.28 (s, 1H), 7.41-7.27 (m, 5H), 7.09 (d, J=1.5 Hz, 2H), 4.59 (s, 4H), 4.15 (s, 2H); ¹³C NMR (126 MHz, CDCl₃) δ 164.49, 138.00, 136.78, 135.00, 130.35, 128.57, 128.17, 128.06, 125.10, 124.56, 121.83, 72.46, 70.59, 42.99.

### N-(2-((Benzyloxy)methyl)-4-chlorophenyl)-2-chloroacetamide (C574)

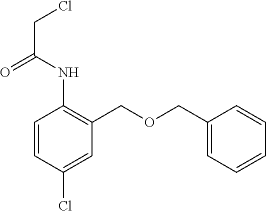 [SMILES: O=C(CCl)Nc1ccc(Cl)cc1COCc1ccccc1]

The title compound was prepared and was isolated as a white solid (393 mg, 95%): ¹H NMR (400 MHz, CDCl₃) δ 9.69 (s, 1H), 8.14 (d, J=8.7 Hz, 1H), 7.41-7.28 (m, 6H), 7.18 (d, J=2.5 Hz, 1H), 4.58 (d, J=10.1 Hz, 4H), 4.14 (s, 2H); ¹³C NMR (101 MHz, CDCl₃) δ 164.46, 136.73, 135.53, 129.63, 129.31, 129.09, 128.72, 128.59, 128.20, 128.05, 123.16, 72.63, 70.62, 43.01.

### N-(2-((Benzyloxy)methyl)-5-bromophenyl)-2-chloroacetamide (C575)

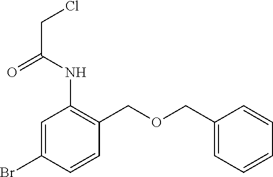 [SMILES: O=C(CCl)Nc1cc(Br)ccc1COCc1ccccc1]

The title compound was prepared and was isolated as a colorless oil (631 mg, 95%): 1H NMR (500 MHz, CDCl₃) δ 9.76 (s, 1H), 8.42 (d, J=1.9 Hz, 1H), 7.40-7.34 (m, 3H), 7.31 (d, J=6.8 Hz, 2H), 7.26-7.22 (m, 1H), 7.03 (d, J=8.1 Hz, 1H), 4.58 (d, J=7.2 Hz, 4H), 4.14 (s, 2H); ¹³C NMR (126 MHz, CDCl₃) δ 164.48, 138.15, 136.77, 130.64, 128.57, 128.18, 128.06, 127.56, 125.62, 124.66, 122.95, 72.47, 70.64, 42.99.

### N-(2-((Benzyloxy)methyl)-5-(trifluoromethyl)phenyl)-2-chloroacetamide (C576)

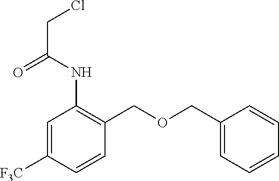 [SMILES: O=C(CCl)Nc1cc(C(F)(F)F)ccc1COCc1ccccc1]

The title compound was prepared and was isolated as a colorless oil (536 mg, 80%): ¹H NMR (500 MHz, CDCl₃) δ 9.88 (s, 1H), 8.55 (s, 1H), 7.41-7.27 (m, 7H), 4.66 (s, 2H), 4.62 (s, 2H), 4.17 (s, 2H); ¹³C NMR (126 MHz, CDCl₃) δ 164.65, 137.56, 136.58, 131.68, 131.42, 130.20, 129.75, 128.61, 128.27, 128.09, 124.82, 122.65, 121.23, 121.20, 118.68, 118.65, 72.75, 70.70, 42.99.

### N-(2-((Benzyloxy)methyl)phenyl)-2-chloroacetamide (C577)

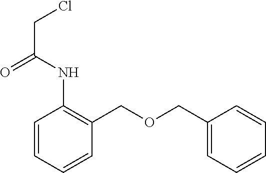 [SMILES: O=C(CCl)Nc1ccccc1COCc1ccccc1]

The title compound was prepared and was isolated as a clear oil (644 mg, 90%): ¹H NMR (500 MHz, CDCl₃) δ 9.71 (s, 1H), 8.17 (d, J=8.1 Hz, 1H), 7.41-7.29 (m, 6H), 7.19 (dd, J=7.5, 1.3 Hz, 1H), 7.12 (td, J=7.5, 1.0 Hz, 1H), 4.61 (d, J=13.5 Hz, 4H), 4.15 (s, 2H); ¹³C NMR (126 MHz, CDCl₃) δ 164.43, 137.09, 136.96, 129.54, 129.31, 128.52, 128.04, 127.01, 124.67, 121.96, 72.40, 71.25, 43.08.

### 2-Chloro-N-(2-methyl-5-(trifluoromethyl)phenyl)acetamide (C578)

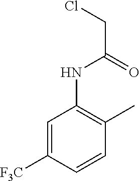 [SMILES: Cc1ccc(C(F)(F)F)cc1NC(=O)CCl]

The title compound was prepared and was isolated as a white solid (689 mg, 95%): ¹H NMR (400 MHz, CDCl₃) δ 8.30 (d, J=13.3 Hz, 1H), 8.26 (s, 1H), 7.41-7.30 (m, 2H), 4.26 (s, 2H), 2.37 (s, 3H); ¹³C NMR (101 MHz, CDCl₃) δ 163.91, 135.16, 132.48, 130.98, 129.66, 129.34, 125.21, 122.26, 122.23, 119.00, 118.96, 43.11, 17.59.

### 2-Chloro-N-(4-methyl-[1,1′-biphenyl]-2-yl)acetamide (C579)

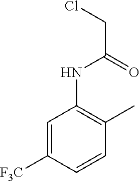 [SMILES: Cc1ccc(C(F)(F)F)cc1NC(=O)CCl]

The title compound was prepared and was isolated as a white solid (731 mg, 98%): ¹H NMR (400 MHz, Chloroform-d) δ 7.71 (d, J=8.1 Hz, 1H), 7.43 (d, J=8.0 Hz, 1H), 7.17 (s, 1H), 4.23-3.98 (m, 2H), 2.46 (s, 3H); ¹³C NMR (101 MHz, CDCl₃) δ 163.59, 138.59, 137.50, 133.66, 129.92, 129.40, 129.08, 128.01, 125.80, 121.21, 43.04, 21.51.

### N-([1,1′-Biphenyl]-2-yl)-2-chloroacetamide (C580)

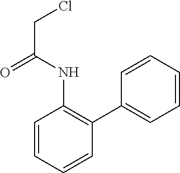 [SMILES: O=C(CCl)Nc1ccccc1-c1ccccc1]

The title compound was prepared and was isolated as a white solid (460 mg, 95%): ¹H NMR (400 MHz, CDCl₃) δ 8.45 (s, 1H), 8.35 (d, J=8.1 Hz, 1H), 7.52-7.47 (m, 2H), 7.46-7.41 (m, 1H), 7.40-7.36 (m, 3H), 7.29 (dd, J=7.6, 1.6 Hz, 1H), 7.22 (td, J=7.5, 1.1 Hz, 1H), 4.07 (s, 2H); ¹³C NMR (101 MHz, CDCl₃) δ 163.63, 137.44, 133.93, 132.67, 130.12, 129.31, 129.14, 128.52, 128.21, 124.96, 120.66, 43.01.

### N-([1,1′-Biphenyl]-3-yl)-2-chloroacetamide (C581)

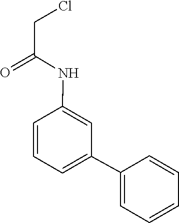 [SMILES: O=C(CCl)Nc1cccc(-c2ccccc2)c1]

The title compound was prepared and was isolated as a white solid (413 mg, 90%): ¹H NMR (500 MHz, CDCl₃) δ 8.30 (s, 1H), 7.78-7.74 (m, 1H), 7.62-7.51 (m, 3H), 7.47-7.32 (m, 5H), 4.21 (s, 2H); ¹³C NMR (101 MHz, CDCl₃) δ 163.98, 142.51, 140.55, 137.25, 129.67, 128.94, 128.76, 127.79, 127.33, 127.24, 124.21, 119.09, 119.02, 43.05.

### N-([1,1′-Biphenyl]-4-yl)-2-chloroacetamide (C582)

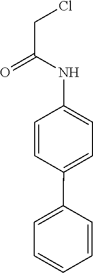 [SMILES: O=C(CCl)Nc1ccc(-c2ccccc2)cc1]

The title compound was prepared and was isolated as a white solid (782 mg, 97%): ¹H NMR (400 MHz, CDCl₃) δ 8.29 (s, 1H), 7.71-7.54 (m, 6H), 7.44 (t, J=7.6 Hz, 2H), 7.34 (t, J=7.3 Hz, 1H), 4.21 (s, 2H); ¹³C NMR (101 MHz, CDCl₃) δ 163.80, 140.29, 138.19, 135.93, 128.84, 127.77, 127.33, 126.92, 120.44, 42.91.

### 2-Chloro-N-(4-fluoro-2,5-dimethylphenyl)acetamide (C583)

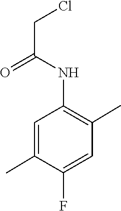

The title compound was prepared and was isolated as a white solid (826 mg, 96%): ¹H NMR (400 MHz, CDCl₃) δ 8.08 (s, 1H), 7.58 (d, J=7.3 Hz, 1H), 6.87 (d, J=9.9 Hz, 1H), 4.23 (s, 2H), 2.24 (s, 6H); ¹³C NMR (101 MHz, CDCl₃) δ 164.09, 160.22, 157.80, 130.17, 130.14, 129.62, 129.54, 126.36, 126.30, 123.30, 123.12, 117.03, 116.80, 43.20, 17.36, 14.44, 14.41.

### 2-Chloro-N-(2,3,5-trimethylphenyl)acetamide (C584)

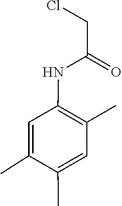

The title compound was prepared and was isolated as a white solid (808 mg, 98%): ¹H NMR (400 MHz, CDCl₃) δ 8.18 (s, 1H), 7.40 (s, 1H), 6.91 (s, 1H), 4.26 (s, 2H), 2.32 (s, 3H), 2.30 (s, 3H), 2.16 (s, 3H); ¹³C NMR (101 MHz, CDCl₃) δ 164.01, 137.34, 135.76, 134.07, 128.94, 125.97, 122.01, 43.16, 20.97, 20.49, 13.22.

### 2-Chloro-N-(2-methoxy-3-methylphenyl)acetamide (C585)

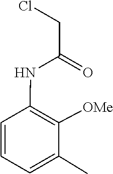

The title compound was prepared and was isolated as a white solid (822 mg, 90%): ¹H NMR (400 MHz, CDCl₃) δ 9.00 (s, 1H), 8.24-8.09 (m, 1H), 7.03 (t, J=7.9 Hz, 1H), 6.98-6.93 (m, 1H), 4.21 (s, 2H), 3.79 (s, 3H), 2.32 (s, 3H); ¹³C NMR (101 MHz, CDCl₃) δ 163.63, 147.58, 130.59, 130.49, 126.95, 124.52, 117.76, 60.41, 43.20, 15.88.

### Example 129: Preparation of 2-(((3-fluorobenzyl)oxy)methyl)-5-methylaniline (C586)

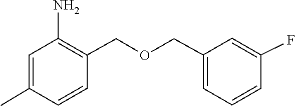 [SMILES: Cc1ccc(COCc2cccc(F)c2)c(N)c1]

1-(((3-Fluorobenzyl)oxy)methyl)-4-methyl-2-nitrobenzene (KE-4a; 400 mg, 1.453 mmol), iron powder (406 mg, 7.27 mmol) and ammonium chloride (38.9 mg, 0.727 mmol) were added to a dried 10-mL vial equipped with a Teflon-coated stirbar and capped with a rubber septum. The flask was evacuated and backfilled with nitrogen three times. Degassed ethanol (3.63 mL) and distilled water (3.63 mL) were added by syringe, and the resulting solution was stirred at 80° C. for 2 h. The reaction mixture was filtered over diatomaceous earth and concentrated in vacuo. The resulting residue was diluted with saturated sodium bicarbonate (50 mL) and DCM (50 mL). The organic layer was separated, dried with magnesium sulfate, and concentrated in vacuo to afford a yellow oil residue (349 mg, 93%). The compound was used without further purification: ¹H NMR (400 MHz, CDCl₃) δ 7.30 (td, J=7.9, 5.8 Hz, 1H), 7.12-7.02 (m, 2H), 7.02-6.91 (m, 2H), 6.57-6.51 (m, 2H), 4.56 (s, 2H), 4.46 (s, 2H), 4.11 (s, 2H), 2.27 (s, 3H); ¹³C NMR (101 MHz, CDCl₃) δ 164.17, 161.73, 146.22, 140.86, 140.79, 139.53, 130.26, 129.96, 129.87, 123.21, 123.18, 118.90, 118.77, 116.53, 114.68, 114.61, 114.46, 114.40, 71.14, 70.43, 21.28.

The following compounds were prepared according to the procedure described in Example 129:

### 5-Methyl-2-(((2-(trifluoromethoxy)benzyl)oxy)methyl)aniline (C587)

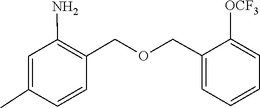 [SMILES: Cc1ccc(COCc2ccccc2OC(F)(F)F)c(N)c1]

The title compound was prepared and was isolated as a yellow oil (361 mg, 94%): ¹H NMR (400 MHz, CDCl₃) δ 7.50 (dd, J=7.3, 2.0 Hz, 1H), 7.38-7.27 (m, 2H), 7.27-7.21 (m, 1H), 6.97 (d, J=7.3 Hz, 1H), 6.54 (d, J=7.7 Hz, 2H), 4.57 (d, J=2.4 Hz, 4H), 4.11 (s, 2H), 2.27 (s, 3H); ¹³C NMR (101 MHz, CDCl₃) δ 146.27, 139.51, 130.85, 130.19, 129.97, 129.02, 126.85, 120.37, 119.05, 118.81, 116.55, 71.62, 65.87, 21.27; ¹⁹F NMR (376 MHz, CDCl₃) δ −57.13.

### 2-(((2,4-Difluorobenzyl)oxy)methyl)-5-methylaniline (C588)

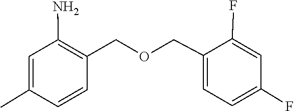 [SMILES: Cc1ccc(COCc2ccc(F)cc2F)c(N)c1]

The title compound was prepared and was isolated as a yellow solid (166 mg, 88%): ¹H NMR (400 MHz, CDCl₃) δ 7.34 (td, J=8.4, 6.4 Hz, 1H), 6.95 (d, J=7.4 Hz, 1H), 6.91-6.75 (m, 2H), 6.58-6.50 (m, 2H), 4.56 (s, 2H), 4.49 (m, 2H), 4.17-4.07 (m, 2H), 2.26 (s, 3H); ¹³C NMR (101 MHz, CDCl₃) δ 162.37, 161.52, 146.29, 139.55, 131.35, 131.29, 131.25, 131.19, 130.23, 121.29, 121.26, 121.14, 121.11, 118.91, 118.80, 116.57, 111.31, 111.28, 111.10, 111.07, 104.08, 103.83, 103.58, 71.39, 64.67, 64.64, 21.27.

### 2-(((4-Fluorobenzyl)oxy)methyl)-5-methylaniline (C589)

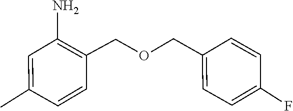 [SMILES: Cc1ccc(COCc2ccc(F)cc2)c(N)c1]

The title compound was prepared and was isolated as a yellow solid (171 mg, 91%): ¹H NMR (400 MHz, CDCl₃) δ 7.34-7.25 (m, 2H), 7.08-6.98 (m, 2H), 6.97-6.90 (m, 1H), 6.57-6.50 (m, 2H), 4.54 (s, 2H), 4.43 (s, 2H), 4.10 (s, 2H), 2.27 (s, 3H); ¹³C NMR (101 MHz, CDCl₃) δ 163.59, 161.15, 146.22, 139.46, 133.91, 133.88, 130.23, 129.74, 129.66, 119.03, 118.76, 116.52, 115.40, 115.18, 70.96, 70.51, 60.42, 21.27.

### 2-(((4-Fluoro-2-(trifluoromethyl)benzyl)oxy)methyl)-5-methylaniline (C590)

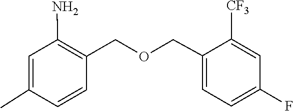 [SMILES: Cc1ccc(COCc2ccc(F)cc2C(F)(F)F)c(N)c1]

The title compound was prepared and was isolated as a yellow oil (350 mg, 91%): ¹H NMR (400 MHz, CDCl₃) δ 7.39-7.31 (m, 2H), 7.22-7.15 (m, 2H), 6.94 (d, J=7.5 Hz, 1H), 6.54 (d, J=7.1 Hz, 1H), 4.56 (s, 2H), 4.46 (s, 2H), 4.10 (s, 2H), 2.27 (s, 3H); ¹³C NMR (101 MHz, CDCl₃) δ 148.67, 146.21, 139.55, 136.94, 130.25, 129.19, 120.96, 118.90, 118.78, 116.54, 71.19, 70.29, 21.27; ¹⁹F NMR (376 MHz, CDCl₃) δ −60.61, −113.14.

### 5-Methyl-2-(((perfluorophenyl)methoxy)methyl)aniline (C591)

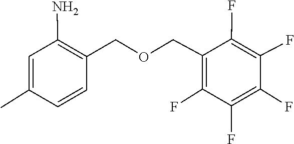 [SMILES: Cc1ccc(COCc2c(F)c(F)c(F)c(F)c2F)c(N)c1]

The title compound was prepared and was isolated as a white solid (234 mg, 81%): ¹H NMR (400 MHz, CDCl₃) δ 6.96 (d, J=7.4 Hz, 1H), 6.59-6.44 (m, 2H), 4.56 (d, J=2.2 Hz, 4H), 4.04 (s, 2H), 2.26 (s, 3H); ¹³C NMR (101 MHz, CDCl₃) δ 146.28, 139.87, 130.29, 118.86, 118.30, 116.61, 111.35, 71.97, 58.19, 21.25.

### 2-(((2-Fluorobenzyl)oxy)methyl)-5-methylaniline (C592)

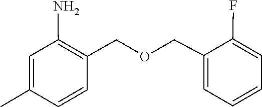 [SMILES: Cc1ccc(COCc2ccccc2F)c(N)c1]

The title compound was prepared and was isolated as a white solid (156 mg, 83%): ¹H NMR (400 MHz, CDCl₃) δ 7.43-7.27 (m, 2H), 7.12 (td, J=7.5, 1.2 Hz, 1H), 7.05 (ddd, J=9.6, 8.1, 1.2 Hz, 1H), 6.96 (d, J=7.4 Hz, 1H), 6.60-6.45 (m, 2H), 4.60-4.53 (m, 4H), 4.12 (s, 2H), 2.26 (s, 3H); ¹³C NMR (101 MHz, CDCl₃) δ 162.22, 159.76, 146.34, 139.46, 130.35, 130.31, 130.23, 129.61, 129.53, 125.25, 125.11, 124.10, 124.07, 119.08, 118.76, 116.55, 115.44, 115.23, 65.22, 65.18, 21.28.

### 2-((Benzyloxy)methyl)-5-methylaniline (C593)

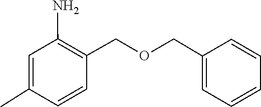 [SMILES: Cc1ccc(COCc2ccccc2)c(N)c1]

The title compound was prepared and was isolated as a clear oil (377 mg, 81%): ¹H NMR (400 MHz, CDCl₃) δ 7.39-7.27 (m, 5H), 6.94 (d, J=7.4 Hz, 1H), 6.57-6.49 (m, 2H), 4.55 (s, 2H), 4.48 (s, 2H), 4.13 (s, 2H), 2.26 (s, 3H); ¹³C NMR (101 MHz, CDCl₃) δ 145.47, 138.55, 137.33, 129.41, 127.63, 127.15, 126.91, 118.41, 117.91, 115.69, 70.48, 70.18, 20.46.

### 2-(((3,5-Bis(trifluoromethyl)benzyl)oxy)methyl)-5-methylaniline (C594)

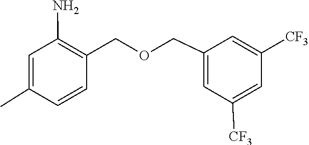 [SMILES: Cc1ccc(COCc2cc(C(F)(F)F)cc(C(F)(F)F)c2)c(N)c1]

The title compound was prepared and was isolated as a yellow oil (346 mg, 89%): ¹H NMR (400 MHz, CDCl₃) δ 7.77 (d, J=9.8 Hz, 3H), 6.95 (d, J=7.4 Hz, 1H), 6.59-6.52 (m, 2H), 4.62 (s, 2H), 4.56 (s, 2H), 4.07 (s, 2H), 2.27 (s, 3H); ¹³C NMR (101 MHz, CDCl₃) δ 146.14, 140.94, 139.93, 131.81, 131.48, 130.38, 127.50, 121.47, 118.89, 118.26, 116.60, 71.70, 69.58, 21.27; ¹⁹F NMR (376 MHz, CDCl₃) δ −62.76.

### 2-(((2-Chlorobenzyl)oxy)methyl)-5-methylaniline (C595)

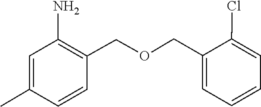 [SMILES: Cc1ccc(COCc2ccccc2Cl)c(N)c1]

The title compound was prepared and was isolated as a yellow oil (340 mg, 90%): ¹H NMR (400 MHz, CDCl₃) δ 7.48-7.41 (m, 1H), 7.39-7.31 (m, 1H), 7.30-7.18 (m, 2H), 6.98 (d, J=7.4 Hz, 1H), 6.58-6.50 (m, 2H), 4.60 (m, 4H), 4.17-4.07 (m, 2H), 2.26 (s, 3H); ¹³C NMR (101 MHz, CDCl₃) δ 146.33, 139.48, 135.81, 133.33, 130.22, 129.52, 129.40, 128.91, 126.82, 119.15, 118.80, 116.56, 71.65, 68.83, 21.28.

### 5-Methyl-2-((thiophen-3-ylmethoxy)methyl)aniline (C596)

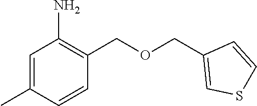 [SMILES: Cc1ccc(COCc2ccsc2)c(N)c1]

The title compound was prepared and was isolated as a yellow oil (134 mg, 68%): ¹H NMR (400 MHz, CDCl₃) δ 7.30 (dd, J=5.0, 3.0 Hz, 1H), 7.23-7.18 (m, 1H), 7.06 (dd, J=5.0, 1.3 Hz, 1H), 6.93 (d, J=7.3 Hz, 1H), 6.53 (d, J=7.6 Hz, 2H), 4.55-4.46 (m, 4H), 2.26 (s, 3H); ¹³C NMR (101 MHz, CDCl₃) δ 146.27, 139.37, 139.25, 130.23, 127.42, 126.05, 123.04, 119.12, 118.72, 116.48, 70.81, 66.35, 21.25.

### 2-((Cyclopropylmethoxy)methyl)-5-methylaniline (C597)

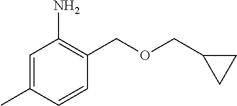

The title compound was prepared and was isolated as a yellow oil (93 mg, 34%): ¹H NMR (400 MHz, CDCl₃) δ 6.92 (d, J=8.0 Hz, 1H), 6.51 (d, J=6.3 Hz, 2H), 4.52 (s, 2H), 4.16 (s, 2H), 3.25 (d, J=6.9 Hz, 2H), 2.25 (s, 3H), 1.14-1.00 (m, 1H), 0.58-0.46 (m, 2H), 0.18 (dt, J=5.9, 4.5 Hz, 2H); ¹³C NMR (101 MHz, CDCl₃) δ 146.27, 139.13, 129.94, 119.57, 118.63, 116.44, 74.10, 71.48, 21.24, 10.63, 2.98.

### 2-((Benzo[d]thiazol-2-ylmethoxy)methyl)-5-methylaniline (C598)

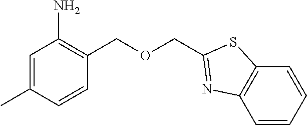 [SMILES: Cc1ccc(COCc2nc3ccccc3s2)c(N)c1]

The title compound was prepared and was isolated as a white solid (257 mg, 90%): ¹H NMR (400 MHz, CDCl₃) δ 8.00 (dt, J=8.1, 0.9 Hz, 1H), 7.89 (dt, J=7.9, 1.0 Hz, 1H), 7.48 (ddd, J=8.2, 7.1, 1.3 Hz, 1H), 7.39 (ddd, J=8.2, 7.1, 1.2 Hz, 1H), 6.99 (d, J=7.9 Hz, 1H), 6.55 (d, J=6.4 Hz, 2H), 4.88 (s, 2H), 4.70 (s, 2H), 4.16 (s, 2H), 2.27 (s, 3H); ¹³C NMR (101 MHz, CDCl₃) δ 169.75, 153.11, 146.27, 139.90, 134.99, 130.57, 126.09, 125.13, 123.06, 121.78, 118.91, 118.20, 116.66, 71.96, 68.57, 21.28.

### 5-Methyl-2-(((1-methyl-3-(trifluoromethyl)-1H-thieno[2,3-c]pyrazol-5-yl)methoxy)methyl)aniline (C599)

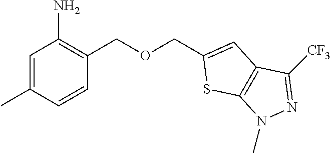 [SMILES: Cc1ccc(COCc2cc3c(C(F)(F)F)nn(C)c3s2)c(N)c1]

The title compound was prepared and was isolated as a white solid (151 mg, 78%): ¹H NMR (400 MHz, CDCl₃) δ 6.94 (d, J=7.3 Hz, 1H), 6.90 (s, 1H), 6.54 (d, J=8.1 Hz, 2H), 4.62-4.58 (m, 2H), 4.57 (s, 2H), 4.08 (s, 2H), 4.02 (s, 3H), 2.27 (s, 3H); ¹³C NMR (101 MHz, CDCl₃) δ 146.22, 145.80, 141.96, 139.75, 132.78, 132.38, 130.40, 126.39, 122.57, 119.91, 118.87, 118.36, 116.60, 114.39, 70.68, 66.43, 38.85, 21.26.

### 2-((Benzo[d]oxazol-2-ylmethoxy)methyl)-5-methylaniline (C600)

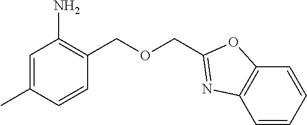 [SMILES: Cc1ccc(COCc2nc3ccccc3o2)c(N)c1]

The title compound was prepared and was isolated as a white solid (110 mg, 83%): ¹H NMR (500 MHz, CDCl₃) δ 7.76-7.70 (m, 1H), 7.56-7.49 (m, 1H), 7.40-7.26 (m, 2H), 7.05-6.93 (m, 1H), 6.53 (d, J=4.9 Hz, 2H), 4.74 (s, 2H), 4.69 (s, 2H), 4.27 (s, 2H), 2.26 (s, 3H); ¹³C NMR (126 MHz, CDCl₃) δ 162.90, 150.89, 146.44, 140.85, 139.96, 130.72, 125.36, 124.52, 120.28, 118.76, 117.91, 116.58, 110.76, 71.97, 63.54, 21.29.

### 2-((Benzyloxy)methyl)-5-chloroaniline (C601)

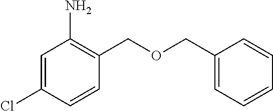 [SMILES: Nc1cc(Cl)ccc1COCc1ccccc1]

The title compound was prepared and was isolated as a yellow oil (300 mg, 80%): ¹H NMR (500 MHz, CDCl₃) δ 7.39-7.25 (m, 5H), 6.96 (d, J=8.5 Hz, 1H), 6.70-6.64 (m, 2H), 4.52 (s, 2H), 4.47 (s, 2H), 4.25 (s, 2H); ¹³C NMR (126 MHz, CDCl₃) δ 147.54, 137.74, 134.79, 131.18, 128.53, 128.01, 127.90, 120.33, 117.67, 115.39, 71.53, 70.55.

### 2-((Benzyloxy)methyl)-4-chloroaniline (C602)

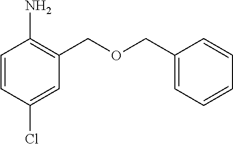 [SMILES: Nc1ccc(Cl)cc1COCc1ccccc1]

The title compound was prepared and was isolated as a yellow oil (254 mg, 90%): ¹H NMR (400 MHz, CDCl₃) δ 7.41-7.26 (m, 5H), 7.13-7.02 (m, 2H), 6.61 (d, J=8.4 Hz, 1H), 4.50 (d, J=10.1 Hz, 4H), 4.16 (s, 2H); ¹³C NMR (101 MHz, CDCl₃) δ 144.92, 137.68, 129.69, 129.01, 128.53, 127.97, 127.91, 123.45, 122.33, 116.84, 71.73, 70.60.

### 2-((Benzyloxy)methyl)-5-bromoaniline (C603)

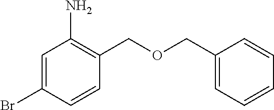 [SMILES: Nc1cc(Br)ccc1COCc1ccccc1]

The title compound was prepared and was isolated as a yellow oil (267 mg, 56%): ¹H NMR (500 MHz, CDCl₃) δ 7.41-7.29 (m, 5H), 6.91 (d, J=8.5 Hz, 1H), 6.82 (d, J=1.9 Hz, 2H), 4.52 (s, 2H), 4.48 (s, 2H), 4.25 (s, 2H); ¹³C NMR (126 MHz, CDCl₃) δ 147.77, 137.74, 131.45, 128.54, 128.02, 127.92, 122.91, 120.79, 120.61, 118.28, 71.54, 70.61.

### 2-((Benzyloxy)methyl)-5-(trifluoromethyl)aniline (C604)

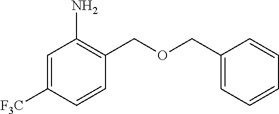 [SMILES: Nc1cc(C(F)(F)F)ccc1COCc1ccccc1]

The title compound was prepared and was isolated as a yellow oil (118 mg, 62%): ¹H NMR (500 MHz, CDCl₃) δ 7.42-7.28 (m, 5H), 7.14 (d, J=7.7 Hz, 1H), 6.94 (d, J=7.8 Hz, 1H), 6.90 (s, 1H), 4.59 (s, 2H), 4.50 (s, 2H), 4.37 (s, 2H); ¹³C NMR (126 MHz, CDCl₃) δ 146.65, 137.56, 131.65, 131.39, 130.32, 128.56, 128.00, 127.99, 125.24, 125.12, 123.07, 114.29, 114.25, 112.07, 112.04, 71.82, 70.65.

### 2-((Benzyloxy)methyl)aniline (C605)

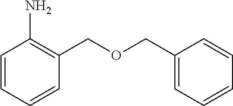 [SMILES: Nc1ccccc1COCc1ccccc1]

The title compound was prepared and was isolated as a yellow oil (111 mg, 57%): ¹H NMR (500 MHz, CDCl₃) δ 7.38-7.27 (m, 5H), 7.14 (td, J=7.7, 1.5 Hz, 1H), 7.06 (dd, J=7.4, 1.2 Hz, 1H), 6.75-6.66 (m, 2H), 4.58 (s, 2H), 4.50 (s, 2H), 4.17 (s, 2H); ¹³C NMR (126 MHz, CDCl₃) δ 146.41, 138.03, 130.21, 129.40, 128.48, 127.98, 127.78, 121.98, 117.89, 115.77, 71.51, 71.26.

### 5-Methyl-2-(((4-(trifluoromethoxy)benzyl)oxy)methyl)aniline (C606)

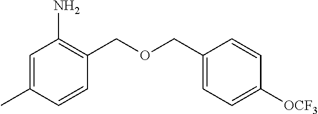 [SMILES: Cc1ccc(COCc2ccc(OC(F)(F)F)cc2)c(N)c1]

The title compound was prepared and was isolated as a white solid (334 mg, 87%): ¹H NMR (400 MHz, CDCl₃) δ 7.63 (dd, J=8.7, 5.5 Hz, 1H), 7.35 (dd, J=9.0, 2.7 Hz, 1H), 7.23 (td, J=8.3, 2.8 Hz, 2H), 6.96 (d, J=7.5 Hz, 1H), 6.55 (d, J=7.1 Hz, 2H), 4.63 (q, J=1.1 Hz, 2H), 4.58 (s, 2H), 4.07 (s, 2H), 2.27 (s, 3H); ¹³C NMR (101 MHz, CDCl₃) δ 162.69, 160.23, 146.14, 139.65, 131.73, 131.65, 130.23, 118.90, 118.85, 118.66, 116.59, 113.36, 71.69, 67.12, 67.09, 21.27; ¹⁹F NMR (376 MHz, CDCl₃) δ −57.92.

### Example 130: Preparation of 3-amino-4-(1-methoxyethyl)benzonitrile (C607)

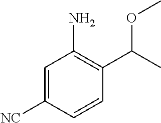

To a solution of 4-(1-methoxyethyl)-3-nitrobenzonitrile (C431; 0.9 g, 4.37 mmol) in THF-EtOH-H₂O (3:2:1 ratio; 25 mL) was added iron powder (1.9 g, 35.0 mmol). Ammonium chloride (2.3 g, 43.68 mmol) was added, and the reaction mixture was stirred at 70° C. for 5 h. The reaction mixture was cooled to room temperature, was filtered through a pad of diatomaceous earth, and washed with EtOAc. The filtrate was concentrated under reduced pressure. The residue was taken up in water and was extracted with EtOAc. The organic layer was washed with brine, dried over anhydrous Na₂SO₄, filtered, and was concentrated under reduced pressure. Purification of the resulting product by column chromatography (silica gel 100-200 mesh) eluting with 10-40% EtOAc in petroleum ether afforded the title compound as a pale brown liquid (0.6 g, 78%): ESIMS m/z 177 ([M+H]⁺).

### Example 131: Preparation of 4-methyl-2-nitro-1-(3-(trifluoromethyl)benzyl)benzene (C608)

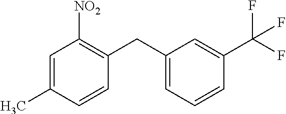 [SMILES: Cc1ccc(Cc2cccc(C(F)(F)F)c2)c([N+](=O)[O-])c1]

4,4,5,5-Tetramethyl-2-(4-methyl-2-nitrophenyl)-1,3,2-dioxaborolane (C654; 0.875 g, 3.33 mmol), 1-(bromomethyl)-3-(trifluoromethyl)benzene (1.192 g, 4.99 mmol), cesium carbonate (3.25 g, 9.98 mmol), and XPhos-Pd-G3 precatalyst (0.056 g, 0.02 equivalents) were combined in dioxane (10 mL) and water (1 mL). The mixture was heated to 85° C. for 12 h. The mixture was cooled, filtered through diatomaceous earth, and the solvents removed. Purification of the residue eluting with a gradient of 0-25% acetone-hexanes provided the title compound as a pale yellow oil (0.600 g, 61%): ¹H NMR (400 MHz, CDCl₃) δ 7.84-7.74 (m, 1H), 7.50-7.46 (m, 1H), 7.44-7.31 (m, 4H), 7.16 (d, J=7.9 Hz, 1H), 4.32 (s, 2H), 2.43 (s, 3H); ¹⁹F NMR (376 MHz, CDCl₃) δ−62.59.

The following compound was prepared in accordance to the procedure in Example 131:

### 1-Benzyl-4-methyl-2-nitrobenzene (C609)

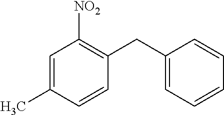 [SMILES: Cc1ccc(Cc2ccccc2)c([N+](=O)[O-])c1]

The title compound was prepared and was isolated as a pale yellow oil (0.555 g, 64%): ¹H NMR (400 MHz, CDCl₃) δ 7.74 (d, J=1.9 Hz, 1H), 7.35-7.16 (m, 4H), 7.17-7.11 (m, 3H), 4.26 (s, 2H), 2.40 (s, 3H); EIMS m/z 227.

### 2-(4-Methyl-2-nitrophenyl)furan (C610)

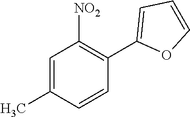

The title compound was prepared and was isolated as a yellow crystalline solid (0.444 g, 57%): ¹H NMR (400 MHz, CDCl₃) δ 7.59 (d, J=8.0 Hz, 1H), 7.51-7.46 (m, 2H), 7.37 (dd, J=8.0, 0.9 Hz, 1H), 6.61 (dd, J=3.4, 0.8 Hz, 1H), 6.48 (dd, J=3.4, 1.8 Hz, 1H), 2.43 (s, 3H); EIMS m/z 203.

### 1-(4-Fluorobenzyl)-4-methyl-2-nitrobenzene (C611)

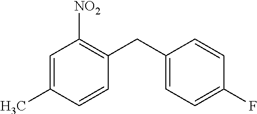 [SMILES: Cc1ccc(Cc2ccc(F)cc2)c([N+](=O)[O-])c1]

The title compound was prepared and was isolated as a yellow oil (323 mg, 85%): ¹H NMR (400 MHz, CDCl₃) δ 7.75 (s, 1H), 7.36-7.30 (m, 1H), 7.14 (d, J=7.9 Hz, 1H), 7.14-7.06 (m, 2H), 7.00-6.91 (m, 2H), 4.22 (s, 2H), 2.41 (s, 3H); ¹⁹F NMR (376 MHz, CDCl₃) δ −116.59.

### 1-(3-Fluorobenzyl)-4-methyl-2-nitrobenzene (C612)

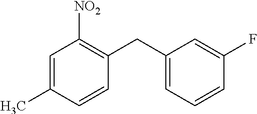 [SMILES: Cc1ccc(Cc2cccc(F)c2)c([N+](=O)[O-])c1]

The title compound was prepared and was isolated as a yellow oil (262 mg, 25%): ¹H NMR (400 MHz, CDCl₃) δ 7.78 (s, 1H), 7.38-7.32 (m, 1H), 7.27-7.19 (m, 1H), 7.16 (d, J=7.8 Hz, 1H), 6.95-6.86 (m, 2H), 6.82 (dt, J=10.0, 2.1 Hz, 1H), 4.26 (s, 2H), 2.42 (s, 3H); ¹⁹F NMR (376 MHz, CDCl₃) δ −113.20.

### 1-(2-Fluorobenzyl)-4-methyl-2-nitrobenzene (C613)

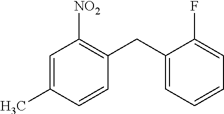 [SMILES: Cc1ccc(Cc2ccccc2F)c([N+](=O)[O-])c1]

The title compound was prepared and was isolated as a yellow oil (400 mg, 48%): ¹H NMR (400 MHz, CDCl₃) δ 7.76 (s, 1H), 7.31 (dd, J=7.9, 1.9 Hz, 1H), 7.26-7.18 (m, 2H), 7.12 (d, J=7.9 Hz, 1H), 7.09-7.02 (m, 2H), 4.29 (s, 2H), 2.40 (s, 3H); ¹⁹F NMR (376 MHz, CDCl₃) 5-117.14.

### 3-(4-Methyl-2-nitrobenzyl)pyridine (C614)

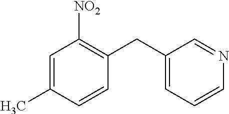 [SMILES: Cc1ccc(Cc2cccnc2)c([N+](=O)[O-])c1]

The title compound was prepared and was isolated as an orange oil (128 mg, 12%): ¹H NMR (400 MHz, CDCl₃) δ 8.50-8.43 (m, 2H), 7.83-7.78 (m, 1H), 7.45 (dt, J=7.8, 2.0 Hz, 1H), 7.37 (dd, J=8.0, 1.9 Hz, 1H), 7.24-7.15 (m, 2H), 4.27 (s, 2H), 2.42 (s, 3H); ¹³C NMR (101 MHz, CDCl₃) δ 149.18, 147.90, 147.00, 137.38, 135.21, 133.62, 133.11, 131.29, 130.46, 124.43, 122.44, 34.55, 19.76.

### Example 132: Preparation of 1-(((3-fluorobenzyl)oxy)methyl)-4-methyl-2-nitrobenzene (C615)

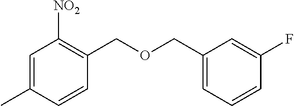 [SMILES: Cc1ccc(COCc2cccc(F)c2)c([N+](=O)[O-])c1]

(4-Methyl-2-nitrophenyl)methanol (C310; 1 g, 5.98 mmol), 1-(bromomethyl)-3-fluorobenzene (2.57 mL, 20.9 mmol) and potassium carbonate (1.24 g, 8.97 mmol) were added to a dried 3-mL vial equipped with a Teflon-coated stirbar and capped with a rubber septum. The vial was evacuated and backfilled with nitrogen three times, and the resulting reaction mixture was stirred at 100° C. for 12 h. The reaction mixture was cooled to room temperature, quenched with distilled water (10 mL), and diluted with DCM (50 mL). The phases were separated, and the organic layer was washed with brine (3×20 mL), dried over magnesium sulfate and concentrated in vacuo to yield a residue. Purification of the residue by flash chromatography (0-30% EtOAc-Hexanes) provided the title compound as a yellow solid (970 mg, 53%): ¹H NMR (400 MHz, CDCl₃) δ 7.89 (dd, J=1.7, 0.9 Hz, 1H), 7.70 (d, J=7.9 Hz, 1H), 7.50-7.43 (m, 1H), 7.33 (tdd, J=8.0, 5.8, 3.9 Hz, 1H), 7.16-7.09 (m, 2H), 7.05-6.97 (m, 1H), 4.92 (s, 2H), 4.64 (s, 2H), 2.44 (s, 3H); ¹³C NMR (101 MHz, CDCl₃) δ 164.35, 161.91, 147.33, 140.65, 140.58, 138.69, 137.57, 134.61, 131.86, 130.41, 130.18, 130.10, 128.82, 125.18, 123.80, 123.10, 123.08, 115.77, 115.56, 115.31, 115.09, 114.90, 114.69, 114.59, 114.37, 77.48, 77.36, 77.16, 76.84, 72.45, 69.16, 69.06, 20.96.

The following compounds were prepared according to the procedure described in Example 132:

### 4-Methyl-2-nitro-1-(((2-(trifluoromethoxy)benzyl)oxy)methyl)benzene (C616)

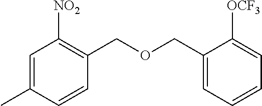 [SMILES: Cc1ccc(COCc2ccccc2OC(F)(F)F)c([N+](=O)[O-])c1]

The title compound was prepared and was isolated as a white solid (1.16 g, 54%): ¹H NMR (400 MHz, CDCl₃) δ 7.92-7.87 (m, 1H), 7.71 (d, J=7.9 Hz, 1H), 7.62-7.53 (m, 1H), 7.46 (dd, J=8.0, 1.8 Hz, 1H), 7.40-7.28 (m, 2H), 7.26 (dt, J=7.5, 1.8 Hz, 1H), 4.96 (s, 2H), 4.72 (s, 2H), 2.44 (s, 3H); ¹³C NMR (101 MHz, CDCl₃) δ 147.61, 147.39, 138.96, 134.93, 132.22, 131.11, 130.05, 129.51, 129.06, 127.43, 127.39, 125.49, 122.31, 120.92, 119.75, 69.75, 67.70, 21.26.

### 1-(((2,4-Difluorobenzyl)oxy)methyl)-4-methyl-2-nitrobenzene (C617)

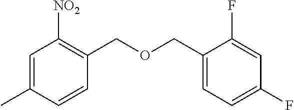 [SMILES: Cc1ccc(COCc2ccc(F)cc2F)c([N+](=O)[O-])c1]

The title compound was prepared and was isolated as a white solid (1.13 g, 61%): ¹H NMR (400 MHz, CDCl₃) δ 7.91-7.85 (m, 1H), 7.67 (d, J=7.9 Hz, 1H), 7.48-7.37 (m, 2H), 6.96-6.77 (m, 2H), 4.92 (s, 2H), 4.67-4.63 (m, 2H), 2.44 (s, 3H); ¹³C NMR (101 MHz, CDCl₃) δ 162.32, 161.45, 147.27, 138.60, 134.42, 131.58, 131.14, 131.09, 131.05, 130.99, 128.74, 125.04, 120.98, 111.44, 111.41, 111.23, 111.20, 104.09, 103.84, 103.58, 69.09, 66.20, 66.17, 20.82.

### 1-(((4-Fluorobenzyl)oxy)methyl)-4-methyl-2-nitrobenzene (C618)

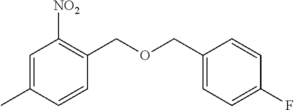 [SMILES: Cc1ccc(COCc2ccc(F)cc2)c([N+](=O)[O-])c1]

The title compound was prepared and was isolated as a yellow solid (1.01 g, 58%): 1H NMR (400 MHz, CDCl₃) δ 7.94-7.85 (m, 1H), 7.68 (d, J=7.9 Hz, 1H), 7.48-7.41 (m, 1H), 7.40-7.30 (m, 2H), 7.10-7.00 (m, 2H), 4.89 (s, 2H), 4.60 (s, 2H), 2.44 (s, 3H); ¹³C NMR (101 MHz, CDCl₃) δ 163.81, 161.36, 147.42, 138.67, 134.53, 133.72, 133.69, 132.35, 131.91, 129.68, 129.60, 128.91, 125.16, 115.61, 115.40, 77.48, 77.16, 76.84, 72.57, 68.99, 20.96.

### 4-Fluoro-1-(((4-methyl-2-nitrobenzyl)oxy)methyl)-2-(trifluoromethyl)benzene (C619)

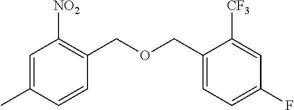 [SMILES: Cc1ccc(COCc2ccc(F)cc2C(F)(F)F)c([N+](=O)[O-])c1]

The title compound was prepared and was isolated as a white solid (1.15 mg, 53%): ¹H NMR (400 MHz, CDCl₃) δ 7.92-7.87 (m, 1H), 7.75-7.64 (m, 2H), 7.49-7.42 (m, 1H), 7.37 (dd, J=9.0, 2.7 Hz, 1H), 7.27 (td, J=8.3, 2.6 Hz, 1H), 4.96 (s, 2H), 4.79 (q, J=1.1 Hz, 2H), 2.45 (s, 3H); ¹³C NMR (101 MHz, CDCl₃) δ 161.99, 159.52, 146.47, 137.96, 133.70, 131.62, 130.60, 130.56, 130.48, 127.95, 124.35, 118.21, 118.00, 112.95, 112.90, 112.70, 112.65, 68.68, 67.78, 67.75, 20.08; ¹⁹F NMR (376 MHz, CDCl₃) δ −60.51, −112.97.

### 1,2,3,4,5-Pentafluoro-6-(((4-methyl-2-nitrobenzyl)oxy)methyl)benzene (C620)

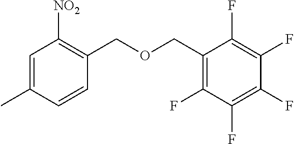 [SMILES: Cc1ccc(COCc2c(F)c(F)c(F)c(F)c2F)c([N+](=O)[O-])c1]

The title compound was prepared and was isolated as a white solid (787 mg, 72%): ¹H NMR (400 MHz, CDCl₃) δ 7.88 (d, J=1.8 Hz, 1H), 7.60 (d, J=7.9 Hz, 1H), 7.44 (dd, J=7.9, 1.8 Hz, 1H), 4.93 (s, 2H), 4.75-4.64 (m, 2H), 2.43 (s, 3H); ¹³C NMR (101 MHz, CDCl₃) δ 147.22, 138.88, 134.46, 130.86, 128.70, 125.11, 69.39, 59.80, 59.60, 20.82; ¹⁹F NMR (376 MHz, CDCl₃) δ −142.59-−142.73 (m), −153.23 (t, J=20.8 Hz), −161.59-−161.79 (m).

### 1-(((2-Fluorobenzyl)oxy)methyl)-4-methyl-2-nitrobenzene (C621)

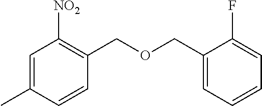 [SMILES: Cc1ccc(COCc2ccccc2F)c([N+](=O)[O-])c1]

The title compound was prepared and was isolated as a yellow oil (1.03 g, 56%): 1H NMR (400 MHz, CDCl₃) δ 7.88 (s, 1H), 7.70 (d, J=7.9 Hz, 1H), 7.46 (t, J=7.6 Hz, 2H), 7.34-7.27 (m, 1H), 7.15 (t, J=7.1 Hz, 1H), 7.06 (t, J=9.1 Hz, 1H), 4.94 (s, 2H), 4.71 (s, 2H), 2.43 (s, 3H), 1.56 (s, 2H); ¹³C NMR (101 MHz, CDCl₃) δ 161.99, 159.54, 147.23, 138.46, 134.43, 131.85, 130.07, 130.02, 129.65, 129.57, 128.72, 125.00, 124.82, 124.19, 124.15, 115.44, 115.23, 69.08, 66.67, 66.64, 20.80; ¹⁹F NMR (376 MHz, CDCl₃) δ −118.73.

### 1-((Benzyloxy)methyl)-4-methyl-2-nitrobenzene (C622)

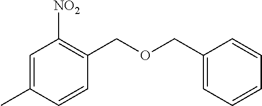 [SMILES: Cc1ccc(COCc2ccccc2)c([N+](=O)[O-])c1]

The title compound was prepared and was isolated as a yellow oil (86 mg, 56%): ¹H NMR (400 MHz, CDCl₃) δ 7.90-7.86 (m, 1H), 7.72 (d, J=8.0 Hz, 1H), 7.48-7.42 (m, 1H), 7.42-7.27 (m, 5H), 4.91 (s, 2H), 4.65 (s, 2H), 2.43 (s, 3H).

### 1-(((3,5-Bis(trifluoromethyl)benzyl)oxy)methyl)-4-methyl-2-nitrobenzene (C623)

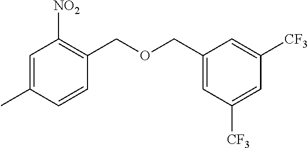 [SMILES: Cc1ccc(COCc2cc(C(F)(F)F)cc(C(F)(F)F)c2)c([N+](=O)[O-])c1]

The title compound was prepared and was isolated as a white solid (1.24 g, 50%): ¹H NMR (400 MHz, CDCl₃) δ 7.93-7.87 (m, 1H), 7.83 (s, 3H), 7.65 (d, J=7.9 Hz, 1H), 7.51-7.44 (m, 1H), 4.98 (s, 2H), 4.75 (s, 2H), 2.45 (s, 3H); ¹³C NMR (101 MHz, CDCl₃) δ 147.34, 140.53, 138.99, 134.51, 132.30, 131.97, 131.64, 130.93, 128.72, 127.34, 127.30, 125.19, 124.64, 121.93, 121.79, 121.75, 121.71, 121.67, 119.22, 71.61, 69.64, 20.85; ¹⁹F NMR (376 MHz, CDCl₃) δ −62.85.

### 1-(((2-Chlorobenzyl)oxy)methyl)-4-methyl-2-nitrobenzene (C624)

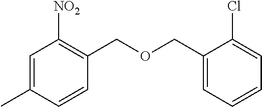 [SMILES: Cc1ccc(COCc2ccccc2Cl)c([N+](=O)[O-])c1]

The title compound was prepared and was isolated as a white solid (1.27 g, 51%): ¹H NMR (400 MHz, CDCl₃) δ 7.94-7.86 (m, 1H), 7.74 (d, J=7.9 Hz, 1H), 7.55 (ddd, J=13.7, 7.4, 1.9 Hz, 1H), 7.46 (dd, J=8.0, 1.6 Hz, 1H), 7.37 (dt, J=7.7, 1.9 Hz, 1H), 7.32-7.26 (m, 2H), 4.99 (s, 2H), 4.74 (s, 2H), 2.44 (s, 3H); ¹³C NMR (101 MHz, CDCl₃) δ 147.08, 138.40, 135.52, 134.41, 132.86, 131.81, 129.77, 129.72, 129.52, 129.29, 129.22, 128.93, 128.90, 128.78, 128.65, 128.58, 126.86, 126.80, 126.75, 124.96, 70.06, 69.22, 20.74.

### 3-(((4-Methyl-2-nitrobenzyl)oxy)methyl)thiophene (C625)

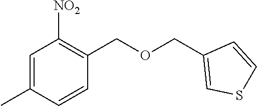 [SMILES: Cc1ccc(COCc2ccsc2)c([N+](=O)[O-])c1]

The title compound was prepared and was isolated as a yellow oil (0.385 g, 44%): ¹H NMR (400 MHz, CDCl₃) δ 7.90-7.84 (m, 1H), 7.68 (d, J=7.9 Hz, 1H), 7.47-7.40 (m, 1H), 7.32 (dd, J=5.0, 2.9 Hz, 1H), 7.27 (s, 1H), 7.11 (dd, J=4.9, 1.3 Hz, 1H), 4.89 (s, 2H), 4.65 (d, J=0.8 Hz, 2H), 2.43 (s, 3H); ¹³C NMR (101 MHz, CDCl₃) δ 147.30, 138.92, 138.43, 134.36, 131.92, 128.87, 128.77, 127.27, 126.15, 124.97, 123.03, 68.72, 68.36, 20.80.

### 1-((Cyclopropylmethoxy)methyl)-4-methyl-2-nitrobenzene (C626)

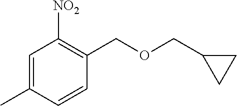 [SMILES: Cc1ccc(COCC2CC2)c([N+](=O)[O-])c1]

The title compound was prepared and was isolated as a yellow oil (0.515 g, 35%): ¹H NMR (400 MHz, CDCl₃) δ 7.90-7.83 (m, 1H), 7.67 (d, J=7.9 Hz, 1H), 7.44 (dd, J=7.7, 1.8 Hz, 1H), 4.85 (s, 2H), 3.40 (d, J=6.9 Hz, 2H), 2.43 (s, 3H), 1.20-1.05 (m, 1H), 0.61-0.48 (m, 2H), 0.29-0.17 (m, 2H); ¹³C NMR (101 MHz, CDCl₃) δ 147.33, 138.27, 134.31, 132.37, 128.73, 124.92, 77.34, 68.95, 20.79, 10.57, 3.01.

### 2-(((4-Methyl-2-nitrobenzyl)oxy)methyl)benzo[d]thiazole (C627)

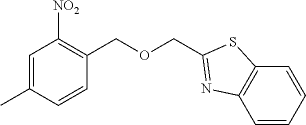 [SMILES: Cc1ccc(COCc2nc3ccccc3s2)c([N+](=O)[O-])c1]

The title compound was prepared and was isolated as a yellow solid (1.13 g, 57%): ¹H NMR (400 MHz, CDCl₃) δ 8.01 (dt, J=8.2, 0.8 Hz, 1H), 7.98-7.87 (m, 2H), 7.75 (d, J=7.9 Hz, 1H), 7.55-7.45 (m, 2H), 7.40 (ddd, J=8.3, 7.2, 1.2 Hz, 1H), 5.07 (d, J=12.1 Hz, 4H), 2.45 (s, 3H); ¹³C NMR (101 MHz, CDCl₃) δ 169.44, 153.25, 147.23, 138.98, 135.16, 134.74, 131.17, 128.78, 126.29, 125.33, 123.26, 121.93, 70.62, 70.09, 20.98.

### 1-Methyl-5-(((4-methyl-2-nitrobenzyl)oxy)methyl)-3-(trifluoromethyl)-1H-thieno[2,3-c]pyrazole (C628)

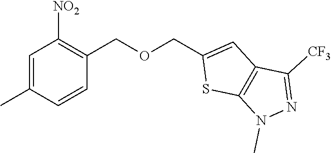 [SMILES: Cc1ccc(COCc2cc3c(C(F)(F)F)nn(C)c3s2)c([N+](=O)[O-])c1]

The title compound was prepared and was isolated as a white solid (679 mg, 40%): ¹H NMR (400 MHz, CDCl₃) δ 7.89 (s, 1H), 7.65 (d, J=7.9 Hz, 1H), 7.46 (d, J=8.0 Hz, 1H), 6.97 (s, 1H), 4.93 (s, 2H), 4.78 (s, 2H), 4.02 (s, 3H), 2.44 (s, 3H); ¹³C NMR (101 MHz, CDCl₃) δ 147.29, 145.83, 141.47, 138.78, 134.43, 131.15, 128.78, 126.36, 125.11, 114.39, 68.72, 68.57, 38.85, 20.82.

### 2-(((4-Methyl-2-nitrobenzyl)oxy)methyl)benzo[d]oxazole (C629)

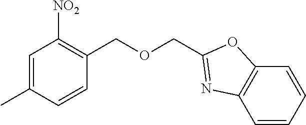 [SMILES: Cc1ccc(COCc2nc3ccccc3o2)c([N+](=O)[O-])c1]

The title compound was prepared and was isolated as a yellow oil (2.17 g, 73%): ¹H NMR (500 MHz, CDCl₃) δ 7.93 (d, J=30.4 Hz, 1H), 7.77-7.70 (m, 2H), 7.58-7.51 (m, 1H), 7.49-7.44 (m, 1H), 7.39-7.31 (m, 2H), 5.08 (s, 2H), 4.90 (s, 2H), 2.43 (s, 3H); ¹³C NMR (101 MHz, CDCl₃) δ 162.17, 154.44, 150.95, 147.15, 140.84, 139.75, 138.81, 134.68, 134.54, 130.90, 128.91, 128.79, 128.47, 125.52, 125.12, 124.57, 120.40, 110.86, 69.94, 66.48, 65.48, 60.39, 21.05, 20.88, 20.81, 14.20.

### 1-((Benzyloxy)methyl)-4-chloro-2-nitrobenzene (C630)

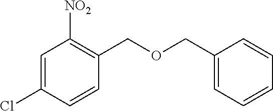 [SMILES: O=[N+]([O-])c1cc(Cl)ccc1COCc1ccccc1]

The title compound was prepared and was isolated as a yellow oil (0.904 g, 58%): ¹H NMR (400 MHz, CDCl₃) δ 8.08 (d, J=2.0 Hz, 1H), 7.84 (d, J=8.4 Hz, 1H), 7.62 (dd, J=8.4, 1.9 Hz, 1H), 7.41-7.34 (m, 5H), 4.92 (s, 2H), 4.67 (s, 2H); ¹³C NMR (101 MHz, CDCl₃) δ 147.43, 137.47, 133.79, 133.73, 130.09, 128.57, 128.42, 128.00, 127.80, 127.75, 124.74, 73.30, 68.39.

### 2-((Benzyloxy)methyl)-4-chloro-1-nitrobenzene (C631)

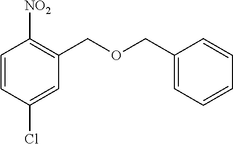 [SMILES: O=[N+]([O-])c1ccc(Cl)cc1COCc1ccccc1]

The title compound was prepared and was isolated as a yellow oil (0.870 g, 57%): ¹H NMR (400 MHz, CDCl₃) δ 8.06 (d, J=8.7 Hz, 1H), 7.90 (dd, J=2.3, 1.1 Hz, 1H), 7.46-7.35 (m, 5H), 7.35-7.28 (m, 1H), 4.94 (d, J=0.9 Hz, 2H), 4.69 (s, 2H); ¹³C NMR (101 MHz, CDCl₃) δ 145.29, 140.82, 137.71, 137.53, 128.87, 128.75, 128.19, 128.11, 127.96, 126.38, 73.52, 68.53.

### 1-((Benzyloxy)methyl)-4-bromo-2-nitrobenzene (C632)

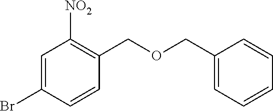 [SMILES: O=[N+]([O-])c1cc(Br)ccc1COCc1ccccc1]

The title compound was prepared and was isolated as a yellow oil (0.804 g, 55%): ¹H NMR (400 MHz, CDCl₃) δ 8.21 (s, 1H), 7.81-7.71 (m, 2H), 7.40-7.29 (m, 5H), 4.89 (s, 2H), 4.66 (s, 2H); ¹³C NMR (101 MHz, CDCl₃) δ 147.52, 137.47, 136.67, 134.32, 130.32, 128.58, 128.42, 128.02, 127.75, 127.58, 121.06, 73.30, 68.44.

### 1-((Benzyloxy)methyl)-2-nitro-4-(trifluoromethyl)benzene (C633)

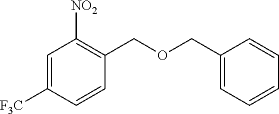 [SMILES: O=[N+]([O-])c1cc(C(F)(F)F)ccc1COCc1ccccc1]

The title compound was prepared and was isolated as a yellow oil (0.770 g, 52%): ¹H NMR (500 MHz, CDCl₃) δ 8.36 (s, 1H), 8.08 (d, J=8.2 Hz, 1H), 7.90 (d, J=7.5 Hz, 1H), 7.39 (d, J=4.4 Hz, 4H), 7.34 (tt, J=8.8, 4.5 Hz, 1H), 5.00 (s, 2H), 4.70 (s, 2H); ¹³C NMR (126 MHz, CDCl₃) δ 146.91, 139.37, 137.30, 130.82, 130.55, 130.12, 130.09, 129.71, 128.62, 128.10, 127.76, 123.97, 122.03, 122.00, 121.80, 73.42, 68.45.

### 1-((Benzyloxy)methyl)-2-nitrobenzene (C634)

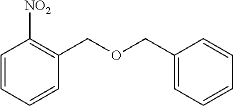 [SMILES: O=[N+]([O-])c1ccccc1COCc1ccccc1]

The title compound was prepared and was isolated as a yellow oil (900 mg, 51%): ¹H NMR (500 MHz, CDCl₃) δ 8.07 (dd, J=8.2, 1.1 Hz, 1H), 7.91-7.84 (m, 1H), 7.65 (td, J=7.7, 1.1 Hz, 1H), 7.46-7.29 (m, 6H), 4.96 (s, 2H), 4.67 (s, 2H); ¹³C NMR (126 MHz, CDCl₃) δ 137.87, 135.27, 133.80, 128.91, 128.73, 128.66, 128.47, 128.08, 128.02, 127.86, 124.81, 73.34, 69.02.

### 4-Methyl-2-nitro-1-(((4-(trifluoromethoxy)benzyl)oxy)methyl)benzene (C635)

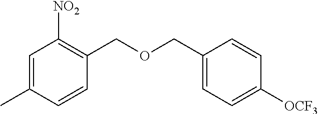 [SMILES: Cc1ccc(COCc2ccc(OC(F)(F)F)cc2)c([N+](=O)[O-])c1]

The title compound was prepared and was isolated as a white solid (1.41 g, 62%): 1H NMR (400 MHz, CDCl₃) δ 7.88 (d, J=1.8 Hz, 1H), 7.68 (d, J=8.0 Hz, 1H), 7.52-7.33 (m, 3H), 7.25-7.16 (m, 2H), 4.92 (s, 2H), 4.64 (s, 2H), 2.44 (s, 3H); ¹³C NMR (101 MHz, CDCl₃) δ 148.92, 147.42, 138.76, 136.73, 134.54, 131.76, 129.13, 129.00, 128.88, 125.19, 121.89, 121.17, 121.05, 119.34, 72.38, 69.23, 20.95; ¹⁹F NMR (376 MHz, CDCl₃) δ −57.86.

### Example 133: Preparation of 1-((2,2-difluoropropoxy)methyl)-4-methyl-2-nitrobenzene (C636)

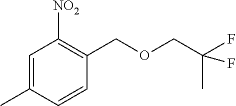 [SMILES: Cc1ccc(COCC(C)(F)F)c([N+](=O)[O-])c1]

To a reaction vial were added 4-methyl-2-nitrobenzyl methanesulfonate (C652; 2.5 g, 10.2 mmol), acetone (34 mL), potassium carbonate (2.82 g, 20.4 mmol), and 2,2-difluoropropan-1-ol (2.48 mL, 30.6 mmol). The reaction mixture was stirred at 40° C. overnight. The reaction mixture was cooled to room temperature and concentrated under reduced pressure. The mixture was diluted with EtOAc and water. The mixture was extracted with EtOAc (2×). The organic extracts were combined, washed with brine, dried over sodium sulfate, filtered over paper, and concentrated under reduced pressure. Purification of the resulting material by column chromatography afforded the title compound as a light yellow oil (1.4 g, 55%): ¹H NMR (400 MHz, CDCl₃) δ 7.89 (dd, J=1.6, 0.9 Hz, 1H), 7.63 (d, J=7.9 Hz, 1H), 7.46 (dd, J=8.0, 1.8 Hz, 1H), 4.96 (s, 2H), 3.72 (t, J=12.0 Hz, 2H), 2.44 (s, 3H), 1.69 (t, J=18.8 Hz, 3H); ¹⁹F NMR (400 MHz, CDCl₃) δ −98.20.

The following compounds were prepared according to the procedure described in Example 133:

### 1-((2-Chloro-2,2-difluoroethoxy)methyl)-4-methyl-2-nitrobenzene (C637)

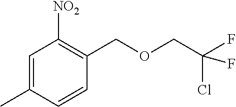 [SMILES: Cc1ccc(COCC(F)(F)Cl)c([N+](=O)[O-])c1]

The title compound was prepared and was isolated as a light yellow oil (800 mg, 70%): ¹H NMR (400 MHz, CDCl₃) δ 7.95-7.89 (m, 1H), 7.67 (d, J=7.9 Hz, 1H), 7.48 (dd, J=8.0, 1.8 Hz, 1H), 5.07 (s, 2H), 4.07 (t, J=11.0 Hz, 2H), 2.45 (s, 4H); ¹⁹F NMR (376 MHz, CDCl₃) δ −61.40.

### Example 134: Preparation of 1-((2,2-difluoroethoxy)methyl)-4-methyl-2-nitrobenzene (C638)

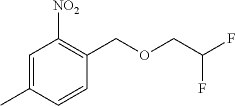 [SMILES: Cc1ccc(COCC(F)F)c([N+](=O)[O-])c1]

To a 250-mL round bottom flask were added THF (45 mL) and sodium hydride (60% suspension in mineral oil; 0.250 g, 6.25 mmol). 2,2-Difluoroethyl trifluoromethanesulfonate (0.984 mL, 7.39 mmol) was added to the suspension, and the reaction mixture was stirred and cooled to 0° C. To the reaction mixture was added a solution of (4-methyl-2-nitrophenyl)methanol (1 g, 5.68 mmol) in THF (45 mL) via an addition funnel. The reaction mixture was stirred at room temperature for 3 h. The reaction mixture was concentrated under reduced pressure. The resulting slurry was dissolved in EtOAc and water. The mixture was extracted was EtOAc twice. The organic extracts were combined, washed with brine, dried over Na₂SO₄, filtered over paper, and concentrated under reduced pressure. Purification of the resulting material by column chromatography afforded the title compound as a yellow oil (1.1 g, 82%): ¹H NMR (400 MHz, CDCl₃) δ 7.95-7.86 (m, 1H), 7.63 (d, J=8.0 Hz, 1H), 7.47 (dt, J=8.0, 1.3 Hz, 1H), 5.95 (tt, J=55.3, 4.0 Hz, 1H), 4.96 (s, 2H), 3.80 (td, J=13.8, 4.0 Hz, 2H), 2.45 (s, 3H); ¹⁹F NMR (376 MHz, CDCl₃) δ −125.10.

### Example 135: Preparation of 1-methyl-3-nitro-2-((2,2,2-trifluoroethoxy)methyl)benzene (C639)

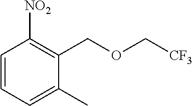 [SMILES: Cc1cccc([N+](=O)[O-])c1COCC(F)(F)F]

To a suspension of NaH (60% suspension in mineral oil; 2.5 g, 59.9 mmol) in THF (150 mL) was added 2,2,2-trifluoroethyl trifluoromethanesulfonate (10.5 g, 44.9 mmol). 2-Methyl-6-nitrophenyl)methanol (C438; 5.0 g, 29.9 mmol) in THF (150 mL) was added dropwise at 0° C., and the reaction mixture was stirred at room temperature for 4 h. The reaction mixture was poured into ice water and was extracted with EtOAc (3×200 mL). The organic layer was washed with brine (50 mL), dried over anhydrous Na₂SO₄, filtered, and was concentrated under reduced pressure. Purification of the resulting product by column chromatography (silica gel 100-200 mesh) eluting with 15-30% EtOAc in petroleum ether afforded the title compound as a pale yellow liquid (5.4 g, 75%): ¹H NMR (300 MHz, DMSO-d₆) δ 7.69 (d, J=7.8 Hz, 1H), 7.58 (d, J=6.9 Hz, 1H), 7.55-7.42 (m, 1H), 4.80 (s, 2H), 4.11 (q, J=9.0 Hz, 2H), 2.45 (s, 3H).

The following compounds were prepared according to the procedure described in Example 135:

### 4-Fluoro-2-nitro-1-((2,2,2-trifluoroethoxy)methyl)benzene (C640)

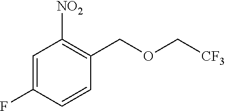 [SMILES: O=[N+]([O-])c1cc(F)ccc1COCC(F)(F)F]

The title compound was prepared and was isolated as a pale yellow liquid (4.8 g, 45%): ¹H NMR (300 MHz, DMSO-d₆) δ 8.02 (dd, J=2.7, 9.0 Hz, 1H), 7.82-7.64 (m, 2H), 4.99 (s, 2H), 4.20 (q, J=9.0 Hz, 2H).

### Example 136: Preparation of 4-methoxy-2-nitro-1-((2,2,2-trifluoroethoxy)methyl)benzene (C641)

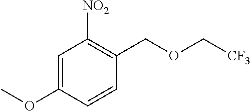 [SMILES: COc1ccc(COCC(F)(F)F)c([N+](=O)[O-])c1]

To a solution of 1-(bromomethyl)-4-methoxy-2-nitrobenzene (C642; 9.0 g, 36.0 mmol) in CH₃CN (100 mL) was added trifluoroethanol (3.0 g, 30.0 mmol). followed by the addition of Cs₂CO₃ (30.0 g, 90 mmol) at 0° C. and the reaction mixture to stirred at room temperature for 5 h. The reaction mixture was poured into water and was extracted with EtOAc (3×300 mL). The organic layer was washed with brine (50 mL), dried over anhydrous Na₂SO₄, filtered and was concentrated under reduced pressure. The crude product was purified by column chromatography (silica gel 100-200 mesh) eluting with 15-30% EtOAc in petroleum ether to afford the title compound as a pale yellow liquid (6.0 g, 61%): ¹H NMR (400 MHz, CDCl₃) δ 7.65 (d, J=8.8 Hz, 1H), 7.61 (d, J=2.8 Hz, 1H), 7.20 (dd, J=2.8, 8.8 Hz, 1H), 4.99 (s, 2H), 3.97 (q, J=8.8 Hz, 2H), 3.89 (s, 3H).

### Example 137: Preparation of 1-(bromomethyl)-4-methoxy-2-nitrobenzene (C642)

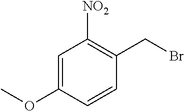

To solution of (4-methoxy-2-nitrophenyl)methanol (C439; 9 g, 49.2 mmol) in DCM (300 mL) was added phosphorus tribromide (PBr₃; 133 g, 491.8 mmol) at 0° C., and the reaction mixture was stirred at room temperature for 16 h. The reaction mixture was quenched with ice water (500 mL), was basified with saturated sodium bicarbonate (pH ˜7), and was extracted with DCM (3×300 mL). The organic layer was washed with water and brine, dried over anhydrous Na₂SO₄, filtered, and concentrated under reduced pressure. The title compound was isolated as a pale yellow liquid which was used in the next step without any purification (9.0 g, 78%): ¹H NMR (300 MHz, DMSO-d₆) δ 7.74-7.63 (m, 1H), 7.59 (d, J=2.7 Hz, 1H), 7.32 (dd, J=2.7, 8.7 Hz, 1H), 4.89 (s, 2H), 3.87 (s, 3H).

### Example 138: Preparation of 4-methyl-2-nitro-1-propoxybenzene (C643)

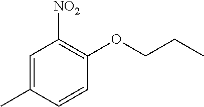

To a solution of 4-methyl-2-nitrophenol (4.0 g, 26.0 mmol) in acetonitrile (50 mL) was added potassium carbonate (7.1 g, 52.0 mmol). 1-Bromopropane (3.5 mL, 39.0 mmol) was added dropwise at 0° C., and the reaction mixture was stirred at 80° C. for 16 h. The reaction mixture was cooled to room temperature, was diluted with water (150 mL), and was extracted with EtOAc (2×200 mL). The organic layer was washed with brine, dried over anhydrous Na₂SO₄, filtered and concentrated under reduced pressure. Purification of the resulting product by silica gel (100-200 mesh) column chromatography eluting with 10-20% EtOAc in petroleum ether afforded the title compound as a pale yellow liquid (4.0 g, 80%): ¹H NMR (300 MHz, DMSO-d₆) δ 7.67 (s, 1H), 7.46-7.43 (m, 1H), 7.23 (d, J=6.0 Hz, 1H), 4.06 (t, J=6.0 Hz, 2H), 2.29 (s, 3H), 1.71-1.68 (m, 2H), 0.67 (t, J=6.0 Hz, 3H); ESIMS m/z 195 ([M]⁺).

### Example 139: Preparation of (3,4-dimethyl-2-nitrophenyl)methanol (C644)

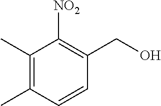

To a solution of methyl 3,4-dimethyl-2-nitrobenzoate (C645; 10.0 g, 47.8 mmol) in THF (200 mL) was added borane dimethyl sulfide complex (BH₃.DMS; 19 mL, 239 mmol) at 0° C., and the reaction mixture was stirred at 70° C. for 16 h. The reaction mixture was cooled to room temperature, was quenched carefully with saturated ammonium chloride (200 mL), and was extracted with EtOAc (2×300 mL). The organic layer was dried over anhydrous Na₂SO₄, filtered, and concentrated under reduced pressure. Purification of the resulting product by silica gel (100-200 mesh) column chromatography eluting with 10-15% EtOAc in petroleum ether afforded the title compound as a pale yellow solid which was used in the next step without any purification (6.5 g, 76%): GC-MS m/z 181 ([M]⁺).

### Example 140: Preparation of methyl 3,4-dimethyl-2-nitrobenzoate (C645)

### Step 1—Preparation of 3,4-dimethyl-2-nitrobenzoic acid (C646)

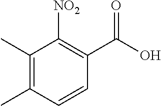

To a solution of 3,4-dimethylbenzoic acid (30.0 g, 200.0 mmol) in H₂SO₄ (220 mL) was added potassium nitrate (KNO₃; 20.2 g, 200.0 mmol) portion wise at 0° C., and the reaction mixture was stirred at 0° C. for 1 h. The reaction mixture was poured into crushed ice and was extracted with DCM (3×500 mL). The organic layer was dried over anhydrous Na₂SO₄, filtered, and concentrated under reduced pressure to afford the title compound as an off-white solid which was used in the next step without any purification (45 g): ESIMS m/z 194 ([M−H]⁻).

### Step 2—Preparation of methyl 3,4-dimethyl-2-nitrobenzoate (C645)

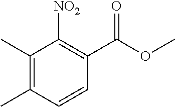

To a solution of 3,4-dimethyl-2-nitrobenzoic acid (C646; 45 g, 230.8 mmol) in acetone (500 mL) was added potassium carbonate (64 g, 461.5 mmol). Dimethyl sulfate (29.1 g, 230.8 mmol) was added at room temperature, and the reaction mixture was stirred at room temperature for 1 h. The reaction mixture was poured into water (200 mL) and was extracted with EtOAc (3×500 mL). The organic layer was dried over anhydrous Na₂SO₄, filtered, and concentrated under reduced pressure. Purification of the resulting product by silica gel (230-400 mesh) column chromatography eluting with 8-10% diethyl ether in petroleum ether afforded the title compound as an off-white solid (11 g, 26% over two steps): ESIMS m/z 210 ([M+H]⁺).

### Example 141: Preparation of 1-(4-methyl-2-nitrophenyl)propan-1-ol (C647)

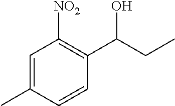

To a solution of 1-iodo-4-methyl-2-nitrobenzene (15 g, 57 mmol) in THF (225 mL) was added phenyl magnesium bromide (1 M in THF; 62.7 mL, 62.7 mmol) at −50° C. dropwise, and the reaction mixture was stirred at −50° C. to −40° C. for 30 minutes. Propanaldehyde (5 mL, 68.4 mmol) was added dropwise at the same temperature (−40° C.), and the reaction mixture was stirred at room temperature for 2 h. The reaction mixture was quenched with saturated ammonium chloride (300 mL). MTBE (500 mL) was added, followed by water (150 mL). The organic layer was washed with brine (200 mL), dried over anhydrous Na₂SO₄, filtered, and was concentrated under reduced pressure. The product was purified by preparative high-performance liquid chromatography. The title compound was prepared and was isolated as a pale yellow liquid (6 g, 48%): ESIMS m/z 178 ([[M+H]-H₂O]⁺).

### Example 142: Preparation of methyl 4-fluoro-2-nitrobenzoate (C648)

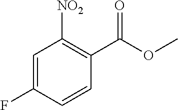

To a solution of 4-fluoro-2-nitrobenzoic acid (25 g, 135 mmol) in DMF (250 mL) was added potassium carbonate (37.2 g, 270 mmol). Iodomethane (57.6 g, 406 mmol) was added at 0° C., and the reaction mixture was stirred at room temperature for 4 h. The reaction mixture was poured into ice water and was extracted with EtOAc (2×200 mL). The organic layer was washed with ice water (100 mL) and brine (100 mL), was dried over anhydrous Na₂SO₄, was filtered and was concentrated under reduced pressure. The title compound was prepared and was isolated as a pale yellow liquid which was used in the next step without any purification (24 g, 89%): ESIMS m/z 200 ([M+H]⁺).

### Example 143: Preparation of methyl 4-(dimethylamino)-2-nitrobenzoate (C649)

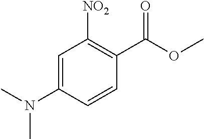

To a stirred solution of methyl 4-fluoro-2-nitrobenzoate (NCG-R1; 10 g, 50.0 mmol) in THF (50 mL) was added dimethyl amine (40% aqueous solution; 50 mL) at room temperature, and the reaction mixture was stirred for 16 h. The reaction mixture was diluted with water (100 mL) and was extracted with EtOAc (2×100 mL). The organic layer was washed with brine (100 mL), was dried over anhydrous Na₂SO₄, was filtered and was concentrated under reduced pressure. The title compound was prepared and was isolated as a yellow solid which was used in the next step without any purification (8.2 g, 73%): ESIMS m/z 225 ([M+H]⁺).

### Example 144: Preparation of 4-(1-hydroxyethyl)-3-nitrobenzonitrile (C650)

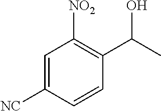

To a solution of 4-iodo-3-nitrobenzonitrile (9 g, 39.7 mmol) in THF (100 mL) was added phenyl magnesium bromide (1 M in THF; 44 mL, 43.6 mmol) at −50° C. drop wise, and the reaction mixture was stirred at −50° C. to −40° C. for 30 minutes. Acetaldehyde (2.8 g, 47.6 mmol) was added drop wise at same temperature (−40° C.), and the reaction mixture was stirred at room temperature for 2 h. The reaction mixture was quenched with saturated ammonium chloride (170 mL). MTBE (130 mL) was added, followed by water (100 mL). The organic layer was washed with brine (200 mL), was dried over anhydrous Na₂SO₄, was filtered and was concentrated under reduced pressure. Purification of the resulting product by column chromatography (silica gel 100-200 mesh) eluting with 15-20% EtOAc in petroleum ether afforded the title compound as a pale yellow (2.8 g, 33%): FT-IR 2238 cm⁻¹ (CN stretching present).

### Example 145: Preparation of (4-(dimethylamino)-2-nitrophenyl)methanol (C651)

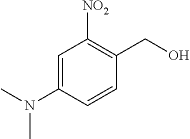

To a solution of methyl 4-(dimethylamino)-2-nitrobenzoate (C649; 10 g, 44.6 mmol) in THF (70 mL) was added diisobutylaluminum hydride (DIBAL-H, 1.0 M in Toluene; 134 mL, 134 mmol) drop wise at −78° C., and the reaction mixture was stirred at room temperature for 16 h. The reaction mixture was quenched with saturated Rochelle salt (potassium sodium tartrate, 100 mL) and was diluted with EtOAc (100 mL). The mixture was stirred at room temperature for 3 h. The mixture was extracted with EtOAc (2×100 mL), was washed with brine (100 mL), was dried over anhydrous Na₂SO₄, was filtered and was concentrated under reduced pressure. Purification of the resulting product by column chromatography (silica gel 100-200 mesh) eluting with 20-40% EtOAc in petroleum ether afforded the title compound as a yellow solid (6 g, 68%): ESIMS m/z 197 ([M+H]⁺).

### Example 146: Preparation of 4-methyl-2-nitrobenzyl methanesulfonate (C652)

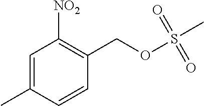

To a 100 mL round bottom flask were added (4-methyl-2-nitrophenyl)methanol (2 g, 12.0 mmol) and DCM (29.9 mL). The reaction mixture was cooled to 0° C., and methanesulfonyl chloride (0.932 mL, 12.0 mmol) and triethylamine (1.67 mL, 12.0 mmol) were added. The reaction mixture was stirred for 2 h. The reaction was quenched with saturated ammonium chloride solution. The reaction mixture was allowed to come to room temperature and was filtered through a phase separator. The filtrate was concentrated under reduced pressure. The title compound was isolated as an orange solid (2.84 g, 92%): ¹H NMR (400 MHz, CDCl₃) δ 7.99 (dd, J=1.7, 0.8 Hz, 1H), 7.61 (d, J=7.9 Hz, 1H), 7.56-7.49 (m, 1H), 5.61 (s, 2H), 3.11 (s, 3H), 2.48 (s, 3H).

### Example 147: Preparation of 1-(2-amino-5-(4,4,5,5-tetramethyl-1,3,2-dioxaborolan-2-yl)phenyl)ethan-1-one (C653)

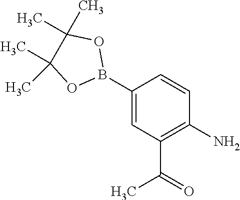 [SMILES: CC(=O)c1cc(B2OC(C)(C)C(C)(C)O2)ccc1N]

1-(2-Amino-5-bromophenyl)ethan-1-one (2.5 g, 11.68 mmol) was combined with bis(pinacolato)diboron (3.26 g, 12.85 mmol), potassium acetate (3.44 g, 35.0 mmol), and [1,1′-bis(diphenylphosphino)ferrocene]dichloropalladium(II) (0.256 g, 0.350 mmol) and dissolved in dioxane (38.9 mL). The mixture was heated to 85° C. for 12 h. The mixture was cooled and filtered through diatomaceous earth. The solvent was removed. Purification of the residue by silica gel chromatography eluting with a gradient of 0-25% acetone-hexanes afforded the title compound as an off-white powder (2.2 g, 72%): ¹H NMR (400 MHz, CDCl₃) δ 8.19 (d, J=1.5 Hz, 1H), 7.67 (dd, J=8.2, 1.5 Hz, 1H), 6.62 (d, J=8.3 Hz, 1H), 6.51 (s, 2H), 2.63 (s, 3H), 1.34 (s, 12H); EIMS m/z 261.

The following compounds were prepared according to the procedure described in Example 147:

### 4,4,5,5-Tetramethyl-2-(4-methyl-2-nitrophenyl)-1,3,2-dioxaborolane (C654)

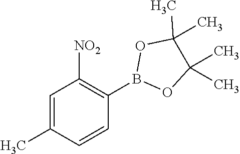

The title compound was prepared and was isolated as a white crystalline solid (4.2 g, 69%): ¹H NMR (400 MHz, CDCl₃) δ 7.99-7.95 (m, 1H), 7.49-7.42 (m, 2H), 2.45 (s, 3H), 1.42 (s, 12H); ESIMS m/z 264 ([M+H]⁺).

The following molecules having a structure according to Formula One and listed in Table 1 and Table 1A may be made according to the aforementioned examples and reported conditions.

TABLE 1

| | |
|---|---|
| H1 | 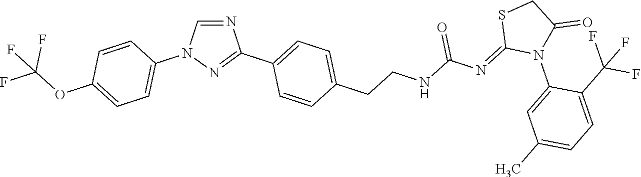 [SMILES: Cc1ccc(C(F)(F)F)c(N2C(=O)CS/C2=N\C(=O)NCCc2ccc(-c3ncn(-c4ccc(OC(F)(F)F)cc4)n3)cc2)c1] |
| H2 | 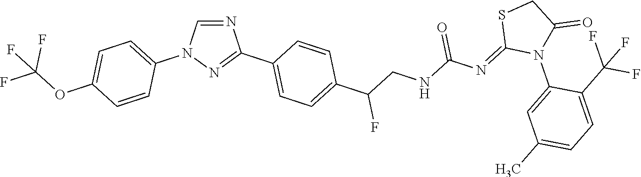 [SMILES: Cc1ccc(C(F)(F)F)c(N2C(=O)CS/C2=N\C(=O)NCC(F)c2ccc(-c3ncn(-c4ccc(OC(F)(F)F)cc4)n3)cc2)c1] |
| H3 | 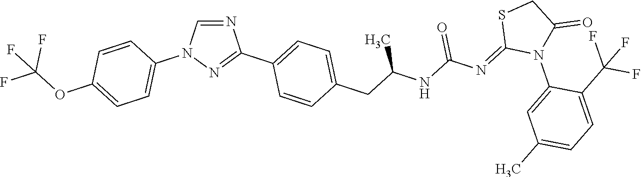 [SMILES: Cc1ccc(C(F)(F)F)c(N2C(=O)CS/C2=N\C(=O)N[C@H](C)Cc2ccc(-c3ncn(-c4ccc(OC(F)(F)F)cc4)n3)cc2)c1] |
| H4 | 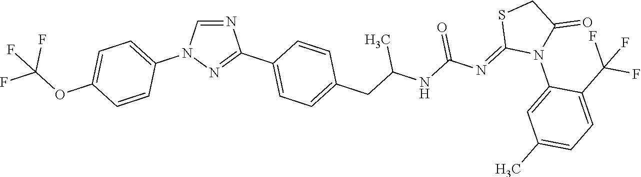 [SMILES: Cc1ccc(C(F)(F)F)c(N2C(=O)CS/C2=N\C(=O)NC(C)Cc2ccc(-c3ncn(-c4ccc(OC(F)(F)F)cc4)n3)cc2)c1] |
| H6 | 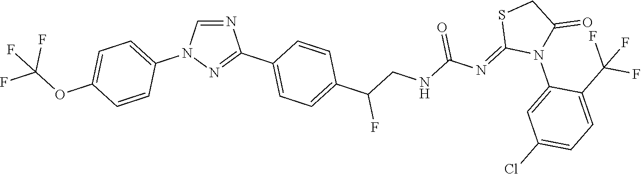 [SMILES: O=C(/N=C1\SCC(=O)N1c1cc(Cl)ccc1C(F)(F)F)NCC(F)c1ccc(-c2ncn(-c3ccc(OC(F)(F)F)cc3)n2)cc1] |
| H7 | 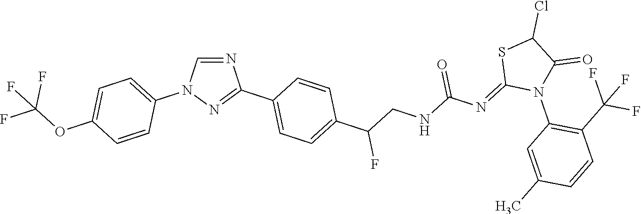 [SMILES: Cc1ccc(C(F)(F)F)c(N2C(=O)C(Cl)S/C2=N\C(=O)NCC(F)c2ccc(-c3ncn(-c4ccc(OC(F)(F)F)cc4)n3)cc2)c1] |
| H8 | 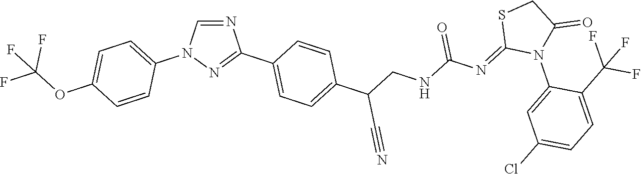 [SMILES: N#CC(CNC(=O)/N=C1\SCC(=O)N1c1cc(Cl)ccc1C(F)(F)F)c1ccc(-c2ncn(-c3ccc(OC(F)(F)F)cc3)n2)cc1] |
| H10 | 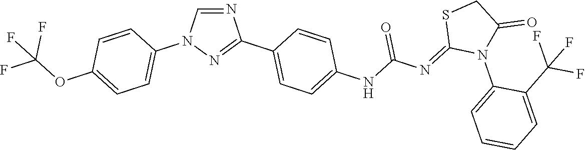 [SMILES: O=C(/N=C1\SCC(=O)N1c1ccccc1C(F)(F)F)Nc1ccc(-c2ncn(-c3ccc(OC(F)(F)F)cc3)n2)cc1] |
| H11 | 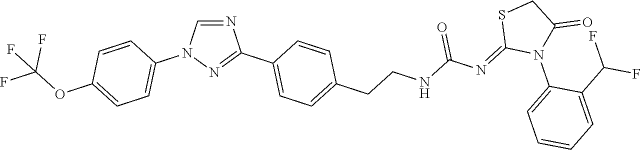 [SMILES: O=C(/N=C1\SCC(=O)N1c1ccccc1C(F)F)NCCc1ccc(-c2ncn(-c3ccc(OC(F)(F)F)cc3)n2)cc1] |
| H12 | 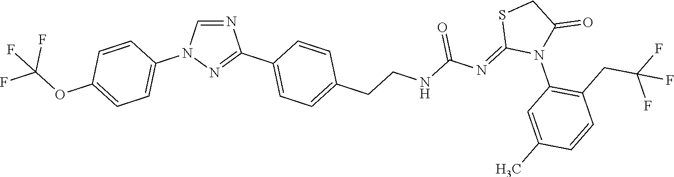 [SMILES: Cc1ccc(CC(F)(F)F)c(N2C(=O)CS/C2=N\C(=O)NCCc2ccc(-c3ncn(-c4ccc(OC(F)(F)F)cc4)n3)cc2)c1] |
| H13 | 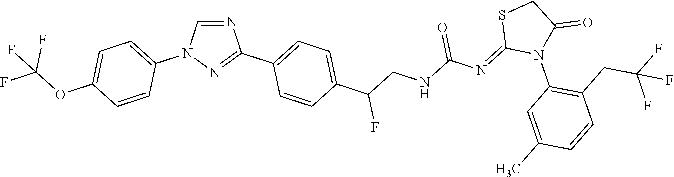 [SMILES: Cc1ccc(CC(F)(F)F)c(N2C(=O)CS/C2=N\C(=O)NCC(F)c2ccc(-c3ncn(-c4ccc(OC(F)(F)F)cc4)n3)cc2)c1] |
| H14 | 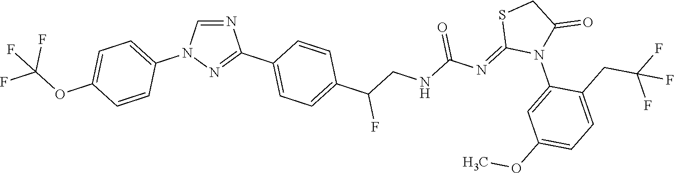 [SMILES: COc1ccc(CC(F)(F)F)c(N2C(=O)CS/C2=N\C(=O)NCC(F)c2ccc(-c3ncn(-c4ccc(OC(F)(F)F)cc4)n3)cc2)c1] |
| H15 | 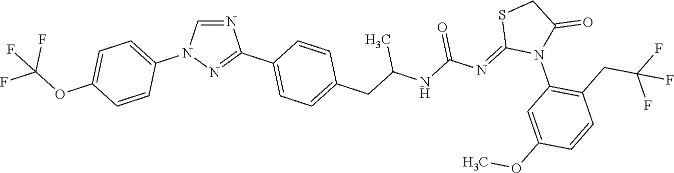 [SMILES: COc1ccc(CC(F)(F)F)c(N2C(=O)CS/C2=N\C(=O)NC(C)Cc2ccc(-c3ncn(-c4ccc(OC(F)(F)F)cc4)n3)cc2)c1] |
| H16 | 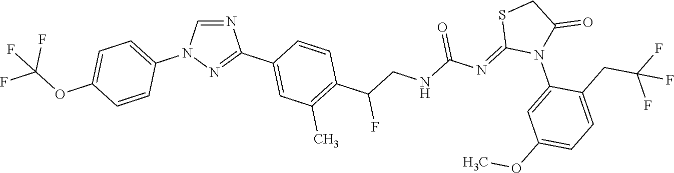 [SMILES: COc1ccc(CC(F)(F)F)c(N2C(=O)CS/C2=N\C(=O)NCC(F)c2ccc(-c3ncn(-c4ccc(OC(F)(F)F)cc4)n3)cc2C)c1] |
| H17 | 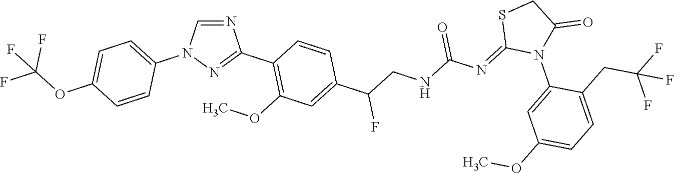 [SMILES: COc1ccc(CC(F)(F)F)c(N2C(=O)CS/C2=N\C(=O)NCC(F)c2ccc(-c3ncn(-c4ccc(OC(F)(F)F)cc4)n3)c(OC)c2)c1] |
| H20 | 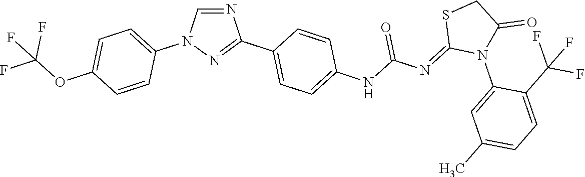 [SMILES: Cc1ccc(C(F)(F)F)c(N2C(=O)CS/C2=N\C(=O)Nc2ccc(-c3ncn(-c4ccc(OC(F)(F)F)cc4)n3)cc2)c1] |
| H21 | 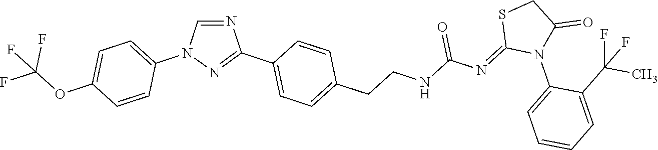 [SMILES: CC(F)(F)c1ccccc1N1C(=O)CS/C1=N\C(=O)NCCc1ccc(-c2ncn(-c3ccc(OC(F)(F)F)cc3)n2)cc1] |
| C1 | 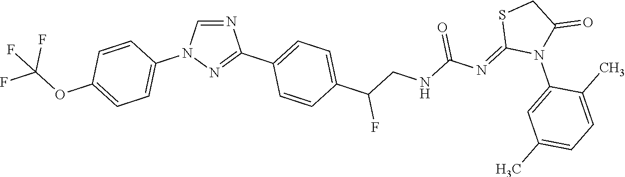 [SMILES: Cc1ccc(C)c(N2C(=O)CS/C2=N\C(=O)NCC(F)c2ccc(-c3ncn(-c4ccc(OC(F)(F)F)cc4)n3)cc2)c1] |
| C2 | 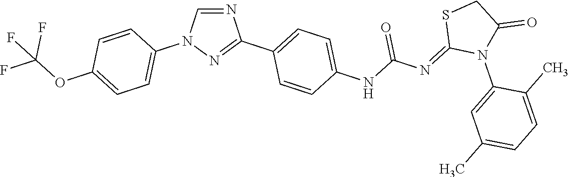 [SMILES: Cc1ccc(C)c(N2C(=O)CS/C2=N\C(=O)Nc2ccc(-c3ncn(-c4ccc(OC(F)(F)F)cc4)n3)cc2)c1] |
| C3 | 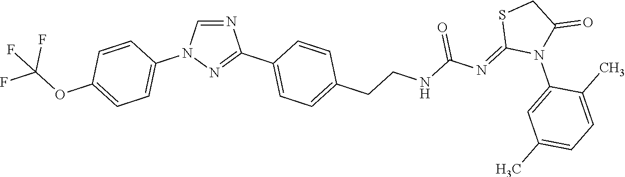 [SMILES: Cc1ccc(C)c(N2C(=O)CS/C2=N\C(=O)NCCc2ccc(-c3ncn(-c4ccc(OC(F)(F)F)cc4)n3)cc2)c1] |

Table 2: Analytical Data for Compounds in Table 1

| | Melting | | |
|---|---|---|---|
| Cmpd. | Point | | |
| No. | (° C.) | MASS SPEC | NMR |
| H1 | | ESIMS m/z 649 | ¹H NMR (400 MHz, CDCl₃) δ 8.55 (s, 1H), 8.12-8.07 (m, |
| | | ([M + H]⁺) | 2H), 7.83-7.75 (m, 2H), 7.66 (d, J = 8.0 Hz, 1H), 7.41- |
| | | | 7.36 (m, 3H), 7.28 (s, 2H), 7.06 (s, 1H), 5.44 (t, J = 6.3 Hz, |
| | | | 1H), 4.00-3.83 (m, 2H), 3.59-3.45 (m, 2H), 2.88 (t, J = |
| | | | 7.1 Hz, 2H), 2.44 (d, J = 1.2 Hz, 3H); ¹⁹F NMR (376 MHz, |
| | | | CDCl₃) δ −58.03, −61.16 |
| H2 | | ESIMS m/z 667 | ¹H NMR (400 MHz, CDCl₃) δ 8.57 (s, 1H), 8.20 (d, J = 8.0 |
| | | ([M + H]⁺) | Hz, 2H), 7.83-7.78 (m, 2H), 7.70 (dd, J = 8.1, 3.4 Hz, 1H), |
| | | | 7.48-7.36 (m, 5H), 7.09 (d, J = 8.0 Hz, 1H), 5.79 (q, J = |
| | | | 7.5 Hz, 1H), 5.61 (ddd, J = 48.4, 8.7, 2.9 Hz, 1H), 4.02- |
| | | | 3.78 (m, 3H), 3.55-3.32 (m, 1H), 2.48 (d, J = 6.4 Hz, 3H); |
| | | | ¹⁹F NMR (376 MHz, CDCl₃) |
| | | | δ −58.03, −61.13, −61.16, −184.31, −184.46 |
| H3 | | ESIMS m/z 663 | ¹H NMR (500 MHz, CDCl₃) δ 8.55 (s, 1H), 8.11-8.05 (m, |
| | | ([M + H]⁺) | 2H), 7.83-7.77 (m, 2H), 7.68 (t, J = 7.6 Hz, 1H), 7.41- |
| | | | 7.37 (m, 3H), 7.28 (d, J = 8.3 Hz, 1H), 7.25 (d, J = 8.2 Hz, |
| | | | 1H), 7.08 (dd, J =10.9, 1.6 Hz, 1H), 5.29 (dd, J = 8.5, 5.7 |
| | | | Hz, 1H), 4.19-4.09 (m, 1H), 3.99-3.82 (m, 2H), 2.97 |
| | | | (ddd, J = 24.0, 13.4, 5.2 Hz, 1H), 2.70 (ddd, J = 28.0, 13.4, |
| | | | 7.8 Hz, 1H), 2.49-2.43 (m, 3H), 1.09 (dd, J = 9.4, 6.7 Hz, |
| | | | 3H); ¹⁹F NMR (471 MHz, CDCl₃) δ −58.03, −61.13, −61.16 |
| H4 | 115-125 | ESIMS m/z 663 | ¹H NMR (400 MHz, CDCl₃) δ 8.55 (s, 1H), 8.08 (dd, J = 8.3, |
| | | ([M + H]⁺) | 2.5 Hz, 2H), 7.83-7.77 (m, 2H), 7.68 (dd, J = 8.1, 5.7 Hz, |
| | | | 1H), 7.39 (dd, J = 9.1, 3.1 Hz, 4H), 7.28-7.24 (m, 1H), |
| | | | 7.11-7.05 (m, 1H), 5.28 (dd, J = 8.2, 4.5 Hz, 1H), 4.13 (q, |
| | | | J = 6.6 Hz, 1H), 4.00-3.83 (m, 2H), 2.97 (ddd, J = 18.7, |
| | | | 13.4, 5.2 Hz, 1H), 2.70 (ddd, J = 21.8, 13.3, 7.8 Hz, 1H), |
| | | | 2.46 (d, J = 7.6 Hz, 3H), 1.09 (t, J = 7.1 Hz, 3H); ¹⁹F NMR |
| | | | (376 MHz, CDCl₃) δ −58.03, −61.13, −61.16 |
| H6 | | ESIMS m/z 685.1 | ¹H NMR (400 MHz, CDCl₃) δ 8.57 (s, 1H), 8.21 (d, J = 8.1 |
| | | ([M − H]⁻) | Hz, 2H), 7.84-7.73 (m, 2H), 7.61 (d, J = 8.8 Hz, 1H), 7.49- |
| | | | 7.36 (m, 4H), 7.32 (d, J = 9.1 Hz, 1H), 5.80 (s, 1H), 5.73- |
| | | | 5.48 (m, 1H), 4.04-3.77 (m, 3H), 3.59-3.33 (m, 2H); |
| | | | ¹⁹F NMR (376 MHz, CDCl₃) δ −58.03, −61.38, −184.39 |
| H7 | | HRMS-ESI (m/z) | ¹H NMR (400 MHz, CDCl₃) δ 8.60 (s, 1H), 8.21 (s, 2H), |
| | | [M + H]⁺ calcd for | 7.91-7.63 (m, 3H), 7.44 (dd, J = 14.7, 8.4 Hz, 5H), 7.16 |
| | | C₂₉H₂₀ClF₇N₆O₃S, | (d, J = 7.9 Hz, 1H), 5.93 (d, J = 25.9 Hz, 1H), 5.85-5.49 |
| | | 701.0967; found, | (m, 2H), 3.86 (d, J = 31.5 Hz, 1H), 3.48 (s, 1H), 2.50 (d, J = |
| | | 701.099 | 5.6 Hz, 3H); ¹⁹F NMR (376 MHz, CDCl₃) |
| | | | δ −58.01, −61.07, −184.42 |
| H8 | | HRMS-ESI (m/z) | ¹H NMR (400 MHz, CDCl₃) δ 8.58 (s, 1H), 8.27-8.15 (m, |
| | | [M + H]⁺ calcd for | 2H), 7.78 (dd, J = 21.7, 8.5 Hz, 2H), 7.61 (d, J = 8.5 Hz, |
| | | C₂₉H₁₈ClF₆N₇O₃S, | 1H), 7.48 (dd, J = 8.3, 3.1 Hz, 2H), 7.40 (d, J = 8.6 Hz, 2H), |
| | | 694.0857; found, | 7.37-7.29 (m, 2H), 5.84 (t, J = 6.6 Hz, 1H), 4.28 (ddd, J = |
| | | 694.0851 | 14.7, 8.9, 5.8 Hz, 1H), 4.05-3.84 (m, 2H), 3.82-3.65 (m, |
| | | | 1H), 3.50 (tdd, J = 14.1, 8.9, 6.0 Hz, 1H); ¹⁹F NMR (376 |
| | | | MHz, CDCl₃) δ −58.02, −61.35 |
| H10 | | ESIMS m/z 607 | ¹H NMR (400 MHz, CDCl₃) δ 8.53 (s, 1H), 8.15-8.09 (m, |
| | | ([M + H]⁺) | 2H), 7.87 (d, J = 7.3 Hz, 1H), 7.82-7.74 (m, 3H), 7.68 (t, J = |
| | | | 7.7 Hz, 1H), 7.60 (d, J = 8.8 Hz, 2H), 7.36 (dd, J = 17.7, |
| | | | 8.1 Hz, 3H), 7.24 (s, 1H), 3.99 (q, J = 18.1 Hz, 2H); ¹⁹F |
| | | | NMR (376 MHz, CDCl₃) δ −58.03, −61.44 |
| H11 | | ESIMS m/z 617.6 | ¹H NMR (400 MHz, CDCl₃) δ 8.53 (s, 1H), 8.11-8.05 (d, |
| | | ([M + H]⁺) | 2H), 7.82-7.75 (d, 2H), 7.73-7.50 (d, 2H), 7.42-7.33 (d, |
| | | | 2H), 7.29-7.23 (m, 2H), 7.21 (d, J = 7.5, 2.0, 1.0 Hz, 1H), |
| | | | 6.57 (t, J = 55.4 Hz, 1H), 5.57 (m, J = 6.1 Hz, 1H), 3.92 (d, |
| | | | J = 4.9 Hz, 2H), 3.58-3.46 (q, 2H), 2.86 (t, J = 7.1 Hz, |
| | | | 2H); ¹³C NMR (101 MHz, CDCl₃) δ 172.04, 168.07, 163.28, |
| | | | 161.88, 161.47, 148.35, 148.33, 141.50, 140.47, 135.55, |
| | | | 131.79, 129.96, 129.52, 129.09, 128.66, 126.79, 126.77, |
| | | | 122.37, 121.16, 41.57, 35.67, 32.95, 17.55 |
| H12 | 185-191 | ESIMS m/z 663 | ¹H NMR (400 MHz, CDCl₃) δ 8.55 (s, 1H), 8.09 (d, J = 8.2 |
| | | ([M + H]⁺) | Hz, 2H), 7.84-7.73 (m, 2H), 7.44-7.33 (m, 3H), 7.27 (d, |
| | | | J = 9.6 Hz, 3H), 7.01-6.94 (m, 1H), 5.48 (t, J = 6.2 Hz, |
| | | | 1H), 3.98-3.83 (m, 2H), 3.58-3.44 (m, 2H), 3.20 (ddd, J = |
| | | | 15.6, 10.4, 5.0 Hz, 2H), 2.88 (t, J = 7.1 Hz, 2H), 2.38 (s, |
| | | | 3H); ¹⁹F NMR (376 MHz, CDCl₃) δ −58.03, −64.42 |
| H13 | 170-181 | ESIMS m/z 681 | ¹H NMR (400 MHz, CDCl₃) δ 8.57 (s, 1H), 8.21 (d, J = 8.0 |
| | | ([M + H]⁺) | Hz, 2H), 7.85-7.77 (m, 2H), 7.45 (d, J = 8.0 Hz, 2H), 7.43- |
| | | | 7.34 (m, 3H), 7.30 (d, J = 8.0 Hz, 1H), 7.07-6.96 (m, |
| | | | 1H), 5.82 (d, J = 7.6 Hz, 1H), 5.62 (d, J = 48.5 Hz, 1H), |
| | | | 4.04-3.87 (m, 1H), 3.48 (q, J = 7.0 Hz, 2H), 3.34-3.15 |
| | | | (m, 2H), 2.41 (d, J = 5.1 Hz, 3H); ¹⁹F NMR (376 MHz, |
| | | | CDCl₃) δ −58.03, −64.45 (d, J = 1.5 Hz), −184.38 (d, J = 27.5 |
| | | | Hz) |
| H14 | | HRMS-ESI (m/z) | ¹H NMR (400 MHz, CDCl₃) δ 8.57 (s, 1H), 8.21 (d, J = 8.0 |
| | | [M + H]⁺ calcd for | Hz, 2H), 7.86-7.74 (m, 2H), 7.45 (d, J = 8.1 Hz, 2H), 7.39 |
| | | C₃₀H₂₃F₇N₆O₄S, | (d, J = 8.6 Hz, 3H), 7.03 (dd, J = 8.6, 2.7 Hz, 1H), 6.71 (dd, |
| | | 697.1462; found, | J = 7.2, 2.6 Hz, 1H), 5.83 (s, 1H), 5.74-5.49 (m, 1H), 4.02- |
| | | 697.1472 | 3.86 (m, 2H), 3.83 (d, J = 5.4 Hz, 3H), 3.47 (dd, J = 15.0, |
| | | | 9.3 Hz, 2H), 3.32-3.05 (m, 2H); ¹³C NMR (126 MHz, |
| | | | CDCl₃) δ 171.82, 168.52, 162.93, 161.75, 160.28, 148.47, |
| | | | 141.61, 138.53, 135.55, 132.89, 130.86, 126.82, 125.71, |
| | | | 122.43, 121.25, 115.92, 114.58, 55.59, 32.96; ¹⁹F NMR |
| | | | (376 MHz, CDCl₃) δ −58.03, −64.74, −184.45 |
| H15 | | HRMS-ESI (m/z) | ¹H NMR (400 MHz, CDCl₃) δ 8.55 (d, J = 1.1 Hz, 1H), 8.09 |
| | | [M + H]⁺ calcd for | (d, J = 7.9 Hz, 2H), 7.86-7.72 (m, 2H), 7.45-7.32 (m, |
| | | C₃₁H₂₆F₆N₆O₄S, | 3H), 7.28 (d, J = 8.0 Hz, 1H), 7.06-6.95 (m, 1H), 6.70 (dd, |
| | | 693.1713; found, | J = 9.1, 2.7 Hz, 1H), 5.40-5.22 (m, 1H), 3.98-3.86 (m, |
| | | 693.1719 | 2H), 3.82 (d, J = 4.0 Hz, 3H), 3.17 (ttd, J = 15.4, 10.7, 5.0 |
| | | | Hz, 2H), 2.96 (ddd, J = 19.4, 13.5, 5.5 Hz, 1H), 2.71 (ddd, J = |
| | | | 26.2, 13.5, 7.7 Hz, 1H), 1.32-1.18 (m, 1H), 1.11 (dd, J = |
| | | | 6.6, 3.6 Hz, 3H), 0.98-0.84 (m, 1H); ¹⁹F NMR (376 MHz, |
| | | | CDCl₃) δ −58.03, −64.67 |
| H16 | | HRMS-ESI (m/z) | ¹H NMR (400 MHz, CDCl₃) δ 8.56 (s, 1H), 8.09-7.98 (m, |
| | | [M + H]⁺ calcd for | 2H), 7.85-7.74 (m, 2H), 7.51-7.34 (m, 4H), 7.03 (dd, J = |
| | | C₃₁H₂₅F₇N₆O₄S, | 8.7, 2.7 Hz, 1H), 6.71 (dd, J = 4.8, 2.6 Hz, 1H), 5.94-5.67 |
| | | 711.1619; found, | (m, 2H), 4.03-3.88 (m, 2H), 3.88-3.75 (m, 4H), 3.48- |
| | | 711.1624 | 3.31 (m, 1H), 3.31-3.09 (m, 2H), 2.43 (d, J = 5.9 Hz, 3H); |
| | | | ¹⁹F NMR (376 MHz, CDCl₃) δ −58.03, −64.75, −185.74 |
| H17 | | HRMS-ESI (m/z) | ¹H NMR (400 MHz, CDCl₃) δ 8.60 (s, 1H), 7.98 (d, J = 8.1 |
| | | [M + H]⁺ calcd for | Hz, 1H), 7.79 (d, J = 9.0 Hz, 2H), 7.39 (dd, J = 8.3, 6.5 Hz, |
| | | C₃₁H₂₅F₇N₆O₅S, | 3H), 7.09-6.95 (m, 2H), 6.71 (dd, J = 7.4, 2.7 Hz, 1H), |
| | | 727.1568; found, | 5.81 (d, J = 7.0 Hz, 1H), 5.74-5.52 (m, 1H), 4.04-3.87 |
| | | 727.1568 | (m, 5H), 3.87-3.74 (m, 3H), 3.49 (dt, J = 17.2, 12.7 Hz, |
| | | | 2H), 3.34-3.04 (m, 3H); ¹⁹F NMR (376 MHz, CDCl₃) |
| | | | δ −58.02, −64.74, −184.96 |
| H20 | | HRMS-ESI (m/z) | ¹H NMR (500 MHz, DMSO-d₆) δ 9.99 (s, 1H), 9.36 (s, 1H), |
| | | [M + H]⁺ calcd for | 8.10-8.03 (m, 1H), 8.03-7.94 (m, 2H), 7.87-7.72 (m, |
| | | C₂₇H₁₈F₆N₆O₃S, | 3H), 7.63-7.59 (m, 2H), 7.56 (d, J = 8.2 Hz, 1H), 7.50 (s, |
| | | 621.1138; found, | 1H), 4.22 (d, J = 18.2 Hz, 1H), 4.09 (d, J = 18.2 Hz, 2H), |
| | | 621.1145 | 2.46 (s, 3H); ¹⁹F NMR (471 MHz, DMSO-d₆) δ −56.96, −59.79 |
| H21 | 90-110 | HRMS-ESI (m/z) | ¹H NMR (500 MHz, CDCl₃) δ 8.55 (s, 1H), 8.08 (d, J = 7.9 |
| | | [M + H]⁺ calcd for | Hz, 2H), 7.83-7.76 (m, 2H), 7.60 (dd, J = 6.8, 2.6 Hz, 1H), |
| | | C₂₉H₂₃F₅N₆O₃S, | 7.56-7.50 (m, 2H), 7.39 (d, J = 8.5 Hz, 2H), 7.30-7.22 |
| | | 630.1473; found, | (m, 4H), 7.17-7.11 (m, 1H), 5.43 (t, J = 6.3 Hz, 1H), 3.89 |
| | | 630.1482 | (q, J = 18.0 Hz, 2H), 3.57-3.44 (m, 2H), 2.93-2.77 (m, |
| | | | 2H), 1.84 (t, J = 18.6 Hz, 3H); ¹³C NMR (126 MHz, CDCl₃) |
| | | | δ 172.52, 168.85, 163.33, 161.66, 148.38, 141.49, 140.55, |
| | | | 135.57, 131.83, 131.10, 130.61, 129.98, 129.11, 128.65, |
| | | | 127.63, 126.77, 122.41, 121.77, 121.25, 121.19, 77.27, |
| | | | 77.22, 77.01, 76.76, 60.40, 41.55, 35.71, 32.94, 26.08, |
| | | | 21.06, 14.20 |
| C1 | | ESIMS m/z 613.2 | ¹H NMR (400 MHz, CDCl₃) δ 8.57 (s, 1H), 8.20 (d, J = 8.0 |
| | | ([M + H]⁺) | Hz, 2H), 7.80 (d, J = 9.0 Hz, 2H), 7.45 (d, J = 8.1 Hz, 2H), |
| | | | 7.42-7.36 (m, 2H), 7.26-7.17 (m, 2H), 6.92 (d, J = 7.9 |
| | | | Hz, 1H), 5.88 (d, J = 3.3 Hz, 1H), 5.62 (dd, J = 48.5, 8.7 Hz, |
| | | | 1H), 3.98-3.89 (m, 2H), 3.84 (dd, J = 19.8, 12.6 Hz, 1H), |
| | | | 3.54-3.35 (m, 1H), 2.37 (d, J = 5.6 Hz, 3H), 2.11 (d, J = |
| | | | 10.1 Hz, 3H); ¹⁹F NMR (400 MHz, CDCls) |
| | | | δ −58.02, −58.03, −184.28, −184.30 |
| C2 | 182-186 | ESIMS m/z 567 | ¹H NMR (400 MHz, DMSO-d₆) δ 9.98 (s, 1H), 9.36 (s, 1H), |
| | | ([M + H]⁺) | 8.10-8.02 (m, 2H), 8.02-7.95 (m, 2H), 7.82-7.74 (m, |
| | | | 2H), 7.67-7.57 (m, 2H), 7.28 (d, J = 7.8 Hz, 1H), 7.20 (dd, |
| | | | J = 8.2, 1.7 Hz, 1H), 7.10 (d, J = 1.8 Hz, 1H), 4.20 (d, J = |
| | | | 17.9 Hz, 1H), 4.09 (d, J = 18.0 Hz, 1H), 2.32 (s, 3H), 2.08 |
| | | | (s, 3H); ¹⁹F NMR (376 MHz, DMSO-d₆) δ −56.96 |
| C3 | | ESIMS m/z 595 | ¹H NMR (400 MHz, CDCl₃) δ 8.55 (s, 1H), 8.11-8.06 (m, |
| | | ([M + H]⁺) | 2H), 7.82-7.75 (m, 2H), 7.41-7.35 (m, 2H), 7.30-7.27 |
| | | | (m, 2H), 7.22-7.13 (m, 2H), 6.91-6.87 (m, 1H), 5.53 (t, J = |
| | | | 6.1 Hz, 1H), 3.91 (d, J = 0.9 Hz, 2H), 3.61-3.46 (m, 2H), |
| | | | 2.88 (t, J = 7.2 Hz, 2H), 2.34 (s, 3H), 2.09 (s, 3H); ¹⁹F NMR |
| | | | (376 MHz, CDCl₃) δ −58.03 |

TABLE 1A

| | | | | | | | | | | | (“Formula Three”) |
|---|---|---|---|---|---|---|---|---|---|---|---|
| 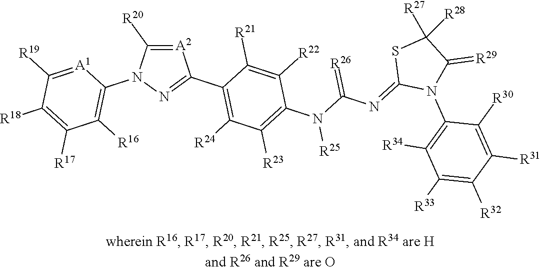 | | | | | | | | | | | |
| Cmpd. | | | | | | | | | | | |
| No. | R¹⁸ | R¹⁹ | A¹ | A² | R²² | R²³ | R²⁴ | R²⁸ | R³⁰ | R³² | R³³ |
| J1 | OCF₃ | H | CH | N | H | CH₃ | H | H | CH(CH₃)₂ | H | CH₃ |
| J2 | OCF₂CF₃ | H | CH | N | H | H | CH₃ | H | CH(CH₃)₂ | H | CH₃ |
| J3 | OCF₃ | H | CH | N | H | OCH₃ | H | H | CH(CH₃)₂ | H | CH₃ |
| J4 | CF₃ | H | CH | N | H | H | CH₃ | H | CH(CH₃)₂ | H | CH₃ |
| J5 | CF₃ | H | CH | N | H | H | CH₃ | H | CH(CH₃)₂ | H | OCH₃ |
| J6 | OCF₃ | H | CH | N | H | OCH₃ | H | H | CH(CH₃)₂ | H | OCH₃ |
| J7 | OCF₂CF₃ | H | CH | N | H | H | CH₃ | H | CH(CH₃)₂ | H | OCH₃ |
| J8 | CF₃ | H | CH | N | H | CH₃ | H | H | CH(CH₃)₂ | H | OCH₃ |
| J9 | CF₃ | H | CH | N | H | CH₃ | H | H | CH(CH₃)₂ | H | CH₃ |
| J10 | CF₃ | H | CH | N | H | OCH₃ | H | H | CH(CH₃)₂ | H | OCH₃ |
| J11 | CF₃ | H | CH | N | H | F | H | H | CH(CH₃)₂ | H | OCH₃ |
| J12 | OCF₂CF₃ | H | CH | N | H | H | CH₃ | H | CH₂CH₂CH₃ | H | CH₃ |
| J13 | CF₃ | H | CH | N | H | OCH₃ | H | H | CH(CH₃)₂ | H | CH₃ |
| J14 | OCF₂CF₃ | H | CH | N | H | H | CH₃ | H | CH₂CH₃ | H | CH₃ |
| J15 | CF₃ | H | CH | N | H | CH₃ | H | H | CH₂CH₂CH₃ | H | CH₃ |
| J16 | CF₃ | H | CH | N | H | CH₃ | H | H | CH₂CH₃ | H | CH₃ |
| J18 | CF₃ | H | CH | N | H | CH₃ | H | H | CH(CH₃)₂ | H | OCH₃ |
| J19 | OCF₃ | H | CH | N | H | F | H | H | CH(CH₃)₂ | H | CH₃ |
| J20 | CF₃ | H | CH | N | H | F | H | H | CH₂CH₂CH₃ | H | CH₃ |
| J21 | CF₃ | H | CH | N | H | F | H | H | CH(CH₃)₂ | H | OCH₃ |
| J22 | OCF₃ | H | CH | N | H | H | CH₃ | H | CH(CH₃)₂ | H | OCH₃ |
| J23 | OCF₃ | H | CH | N | H | H | CH₃ | H | CH(CH₃)₂ | H | CH₃ |
| J24 | OCF₂CF₃ | H | CH | N | H | CH₃ | H | H | CH(CH₃)₂ | H | CH₃ |
| J25 | OCF₂CF₃ | H | CH | N | H | CH₃ | H | H | CH(CH₃)₂ | H | OCH₃ |
| J26 | OCF₂CF₃ | H | CH | N | H | F | H | H | CH(CH₃)₂ | H | CH₃ |
| J27 | OCF₂CF₃ | H | CH | N | H | F | H | H | CH(CH₃)₂ | H | OCH₃ |
| J28 | OCF₂CF₃ | H | CH | N | H | OCH₃ | H | H | CH(CH₃)₂ | H | CH₃ |
| J29 | OCF₂CF₃ | H | CH | N | H | OCH₃ | H | H | CH(CH₃)₂ | H | OCH₃ |
| J30 | OCF₃ | H | CH | N | H | F | H | H | CF₃ | H | CH₃ |
| J31 | OCF₃ | H | CH | N | H | F | H | H | OCH₂CF₃ | H | CH₃ |
| J32 | OCF₃ | H | CH | N | H | F | H | Cl | CH(CH₃)₂ | H | CH₃ |
| J33 | OCF₃ | H | CH | N | H | F | H | Cl | OCH₂CF₃ | H | CH₃ |
| J34 | OCF₃ | H | CH | N | H | F | H | Cl | CF₃ | H | CH₃ |
| J35 | OCF₃ | H | CH | N | H | F | H | H | O(CH₂)₂CF₃ | H | CH₃ |
| J36 | OCF₃ | H | CH | N | H | F | H | Cl | O(CH₂)₂CF₃ | H | CH₃ |
| J37 | OCF₃ | H | CH | N | H | Cl | H | H | CH(CH₃)₂ | H | OCH₃ |
| J38 | OCF₃ | H | CH | N | H | Cl | H | H | CH(CH₃)₂ | H | CH₃ |
| J39 | OCF₃ | H | CH | N | H | Cl | H | H | CF₃ | H | CH₃ |
| J40 | OCF₃ | H | CH | N | H | Cl | H | H | OCH₂CF₃ | H | CH₃ |
| J41 | OCF₃ | H | CH | N | H | F | H | H | CH₂CH₂CH₃ | H | CH₃ |
| J42 | OCF₃ | H | CH | N | H | Cl | H | H | CF₃ | H | Cl |
| J43 | OCF₃ | H | CH | N | H | F | H | H | CH₂CH₂CH₃ | H | OCH₃ |
| J44 | OCF₃ | H | CH | N | H | F | H | H | CH₂CH₂CH₂CH₃ | H | CH₃ |
| J45 | OCF₃ | H | CH | N | H | F | H | H | CH₂CH₂CH₂CH₃ | H | OCH₃ |
| J46 | OCF₃ | H | CH | N | H | F | H | H | CH(CH₃)₂ | H | Cl |
| J47 | OCF₃ | H | CH | N | H | F | H | H | CH₂CH(CH₃)₂ | H | CH₃ |
| J48 | OCF₃ | H | CH | N | H | F | H | H | CH(CH₃)CH₂CH₃ | H | OCH₃ |
| J49 | OCF₃ | H | CH | N | H | F | H | H | CH(CH₃)OCH₃ | H | CH₃ |
| J50 | OCF₃ | H | CH | N | H | F | H | H | CF₃ | H | CF₃ |
| J51 | OCF₃ | H | CH | N | H | F | H | H | OCH(CH₃)CH₂CH₃ | H | CH₃ |
| J52 | OCF₂CF₃ | H | CH | N | H | F | H | H | CH₂CH₂CH₃ | H | CH₃ |
| J53 | OCF₃ | H | CH | N | H | CH₃ | H | H | CH₂CH₂CH₃ | H | CH₃ |
| J54 | OCF₃ | H | CH | N | H | CH₃ | H | H | CH(CH₃)₂ | H | OCH₃ |
| J55 | OCF₃ | H | CH | N | H | CH₃ | H | H | OCH₂CF₃ | H | CH₃ |
| J56 | OCF₃ | H | CH | N | H | CH₃ | H | H | O(CH₂)₂CF₃ | H | CH₃ |
| J57 | OCH₂CF₃ | H | CH | N | H | F | H | H | CH(CH₃)₂ | H | CH₃ |
| J58 | OCH₂CF₃ | H | CH | N | H | F | H | H | CH(CH₃)₂ | H | OCH₃ |
| J59 | OCF₃ | H | CH | N | H | F | H | H | OCH₂CH₃ | H | CH₃ |
| J60 | OCF₃ | H | CH | N | H | CH₃ | H | H | CH(CH₃)OCH₃ | H | CH₃ |
| J61 | CF₃ | H | CH | N | H | F | H | H | CH(CH₃)₂ | H | CH₃ |
| J62 | CF₃ | H | CH | N | H | F | H | H | CH(CH₃)OCH₃ | H | CH₃ |
| J63 | CF₃ | H | CH | N | H | F | H | H | OCH₂CF₃ | H | CH₃ |
| J64 | CF₃ | H | CH | N | H | F | H | H | O(CH₂)₂CF₃ | H | CH₃ |
| J65 | CF₃ | H | CH | N | H | F | H | H | CH(CH₃)CH₂CH₃ | H | CH₃ |
| J66 | OCF₃ | H | CH | N | H | F | H | H | CH₂OCH₃ | H | CH₃ |
| J67 | OCF₃ | H | CH | N | H | CH₃ | H | H | CH₂OCH₃ | H | CH₃ |
| J68 | OCF₃ | H | CH | N | H | CH₃ | H | H | CH(CH₃)₂ | H | N(CH₃)₂ |
| J69 | OCF₃ | H | CH | N | H | F | H | H | CH(CH₃)₂ | H | N(CH₂CH₃)₂ |
| J70 | OCF₃ | H | CH | N | H | CH₂CH₃ | H | H | CH(CH₃)₂ | H | CH₃ |
| J71 | OCF₃ | H | CH | N | H | CH₂CH₃ | H | H | CH(CH₃)₂ | H | OCH₃ |
| J72 | OCF₃ | H | CH | N | H | CH₂CH₃ | H | H | CH₂OCH₃ | H | CH₃ |
| J73 | OCF₃ | H | CH | N | H | CH₂CH₃ | H | H | O(CH₂)₂CF₃ | H | CH₃ |
| J74 | OCF₃ | H | CH | N | H | CH₂CH₃ | H | H | CH(CH₃)OCH₃ | H | CH₃ |
| J75 | OCF₃ | H | CH | N | H | CH₂CH₃ | H | H | OCH₂CF₃ | H | CH₃ |
| J76 | OCF₃ | H | CH | N | H | F | H | H | CH(CH₃)₂ | H | CN |
| J77 | OCF₃ | H | CH | N | H | F | H | H | CH(CH₃)₂ | H | C(═O)CH₃ |
| J78 | OCF₃ | H | CH | N | F | F | H | H | CH(CH₃)₂ | H | CH₃ |
| J79 | OCF₃ | H | CH | N | F | F | H | H | OCH₂CF₃ | H | CH₃ |
| J80 | OCF₃ | H | CH | N | F | F | H | H | O(CH₂)₃CF₃ | H | CH₃ |
| J81 | OCF₃ | H | CH | N | F | F | H | H | CH(CH₃)OCH₃ | H | CH₃ |
| J82 | OCF₃ | H | CH | N | F | F | H | H | CH(CH₃)₂ | H | N(CH₃)₂ |
| J83 | OCF₃ | H | CH | N | H | CH₂CH₃ | H | H | CH(CH₃)₂ | H | N(CH₃)₂ |
| J84 | OCF₃ | H | CH | N | H | F | H | H | H | H | N(CH₃)₂ |
| J85 | OCF₃ | H | CH | N | H | F | H | H | CH(CH₃)₂ | H | NHCH₃ |
| J86 | OCF₃ | H | CH | N | H | F | H | H | CH(CH₃)₂ | H | H |
| J87 | OCF₃ | H | CH | N | H | F | H | H | CH(CH₃)OCH₃ | H | H |
| J88 | OCF₃ | H | CH | N | H | F | H | H | CH(CH₃)—S—CH₃ | H | H |
| J89 | OCF₃ | H | CH | N | H | OCH₃ | H | H | CH(CH₃)₂ | H | N(CH₃)₂ |
| J90 | OCF₃ | H | CH | N | H | OCH₃ | H | H | CH(CH₃)OCH₃ | H | CH₃ |
| J91 | OCF₃ | H | CH | N | H | OCH₃ | H | H | O(CH₂)₂CF₃ | H | CH₃ |
| J92 | OCF₃ | H | CH | N | H | F | H | H | O(CH₂)₂CF₃ | H | OCH₃ |
| J93 | OCF₃ | H | CH | N | H | CH(CH₃)₂ | H | H | O(CH₂)₂CF₃ | H | CH₃ |
| J94 | OCF₃ | H | CH | N | H | CH(CH₃)₂ | H | H | CH(CH₃)OCH₃ | H | CH₃ |
| J95 | OCF₃ | H | CH | N | CH₃ | CH₃ | H | H | CH(CH₃)₂ | H | CH₃ |
| J96 | OCF₃ | H | CH | N | CH₃ | CH₃ | H | H | CH(CH₃)₂ | H | N(CH₃)₂ |
| J97 | OCF₃ | H | CH | N | CH₃ | CH₃ | H | H | CH(CH₃)OCH₃ | H | CH₃ |
| J98 | OCF₃ | H | CH | N | CH₃ | CH₃ | H | H | CH(CH₃)₂ | H | OCH₃ |
| J99 | OCF₃ | H | CH | N | H | CH(CH₃)₂ | H | H | CH(CH₃)₂ | H | CH₃ |
| J100 | OCF₃ | H | CH | N | H | CH(CH₃)₂ | H | H | CH(CH₃)₂ | H | OCH₃ |
| J101 | OCF₃ | H | CH | N | H | CH(CH₃)₂ | H | H | CH(CH₃)₂ | H | N(CH₃)₂ |
| J102 | OCF₃ | H | CH | N | H | CH₃ | H | H | O(CH₂)₂CF₃ | H | N(CH₃)₂ |
| J103 | OCF₃ | H | CH | N | H | F | H | H | O(CH₂)₂CF₃ | H | CN |
| J104 | OCF₃ | H | CH | N | H | F | H | H | CH(CH₃)₂ | H | C(═O)OCH₃ |
| J105 | OCF₃ | H | CH | N | H | F | H | H | O(CH₂)₂CF₃ | H | N(CH₃)₂ |
| J106 | OCF₃ | H | CH | N | H | F | H | H | CH(CH₃)₂ | H | NH(CH₂CH₃) |
| J107 | OCF₃ | H | CH | N | H | F | H | H | CH(CH₃)₂ | H | NH(CH₂CH₂CH₃) |
| J108 | OCF₃ | H | CH | N | H | F | H | H | CH(CH₃)₂ | H | N(CH₂CH₂CH₃)₂ |
| J109 | OCF₃ | H | CH | N | H | OCH₃ | H | H | O(CH₂)₂CF₃ | H | N(CH₃)₂ |
| J110 | OCF₃ | H | CH | N | H | CH₂CH₃ | H | H | O(CH₂)₂CF₃ | H | N(CH₃)₂ |
| J111 | OCF₃ | H | CH | N | H | CH(CH₃)₂ | H | H | O(CH₂)₂CF₃ | H | N(CH₃)₂ |
| J112 | OCF₃ | H | CH | N | H | F | H | H | CH(CH₃)₂ | N(CH₃)₂ | H |
| J113 | OCF₃ | H | CH | N | H | CH₃ | H | H | CH(CH₃)₂ | N(CH₃)₂ | H |
| J114 | OCF₃ | H | CH | N | H | CF₃ | H | H | CH(CH₃)₂ | H | CH₃ |
| J115 | OCF₃ | H | CH | N | H | CF₃ | H | H | OCH₂CF₃ | H | CH₃ |
| J116 | OCF₃ | H | CH | N | H | CF₃ | H | H | CH(CH₃)₂ | H | N(CH₃)₂ |
| J117 | OCF₃ | H | CH | N | H | CF₃ | H | H | CH(CH₃)OCH₃ | H | CH₃ |
| J118 | OCF₃ | H | CH | N | H | CF₃ | H | H | O(CH₂)₂CF₃ | H | N(CH₃)₂ |
| J119 | H | CF₃ | CH | N | H | F | H | H | CH(CH₃)OCH₃ | H | CH₃ |
| J120 | H | OCF₃ | CH | N | H | F | H | H | CH(CH₃)OCH₃ | H | CH₃ |
| J121 | H | OCF₃ | CH | N | H | F | H | H | OCH₂CF₃ | H | CH₃ |
| J122 | H | CF₃ | CH | N | H | F | H | H | OCH₂CF₃ | H | CH₃ |
| J123 | OCF₃ | H | CH | N | H | F | H | H | CH₂OCH₂CF₃ | H | CH₃ |
| J124 | OCF₃ | H | CH | N | H | F | H | H | CH(CH₃)OCH₂CH₃ | H | CH₃ |
| J125 | OCF₃ | H | CH | N | H | F | H | H | O(CH₂)₂CH₃ | H | CH₃ |
| J126 | OCF₃ | H | CH | N | H | CH₃ | H | H | CH(CH₃)₂ | H | NHCH₃ |
| J127 | OCF₃ | H | CH | N | H | CH₃ | H | H | CH(CH₃)₂ | H | NH(CH₂CH₃) |
| J128 | OCF₃ | H | CH | N | H | CH₃ | H | H | CH(CH₃)₂ | H | NH(CH₂CH₂CH₃) |
| J129 | OCF₃ | H | CH | N | H | CH₃ | H | H | CH(CH₃)₂ | H | N(CH₂CH₃)₂ |
| J130 | OCF₃ | H | CH | N | H | CH₃ | H | H | CH(CH₃)₂ | H | N(CH₂CH₂CH₃)₂ |
| J131 | H | CF₃ | CH | N | H | CH₃ | H | H | CH(CH₃)₂ | H | CH₃ |
| J132 | H | CF₃ | CH | N | H | CH₃ | H | H | CH(CH₃)₂ | H | OCH₃ |
| J133 | H | CF₃ | CH | N | H | CH₃ | H | H | CH(CH₃)₂ | H | N(CH₃)₂ |
| J134 | H | CF₃ | CH | N | H | CH₃ | H | H | O(CH₂)₂CF₃ | H | CH₃ |
| J135 | H | OCF₃ | CH | N | H | CH₃ | H | H | CH(CH₃)₂ | H | CH₃ |
| J136 | H | OCF₃ | CH | N | H | CH₃ | H | H | CH(CH₃)₂ | H | OCH₃ |
| J137 | H | OCF₃ | CH | N | H | CH₃ | H | H | CH(CH₃)₂ | H | N(CH₃)₂ |
| J138 | H | OCF₃ | CH | N | H | CH₃ | H | H | O(CH₂)₂CF₃ | H | CH₃ |
| J139 | OCF₃ | H | CH | N | H | CH₃ | H | H | CH₂OCH₂CF₃ | H | CH₃ |
| J140 | H | CF₃ | CH | N | H | F | H | H | CH(CH₃)₂ | H | CH₃ |
| J141 | OCF₃ | H | CH | N | H | CH₃ | H | H | CH(CH₃)OCH₂CH₃ | H | CH₃ |
| J142 | H | CF₃ | CH | N | H | F | H | H | O(CH₂)₂CF₃ | H | CH₃ |
| J143 | H | OCF₃ | CH | N | H | F | H | H | CH(CH₃)₂ | H | CH₃ |
| J144 | OCH₃ | H | CH | N | H | F | H | H | CH(CH₃)₂ | H | CH₃ |
| J145 | OCH₃ | H | CH | N | H | F | H | H | CH(CH₃)₂ | H | OCH₃ |
| J146 | OCH₃ | H | CH | N | H | F | H | H | CH(CH₃)₂ | H | N(CH₃)₂ |
| J147 | OCH₃ | H | CH | N | H | F | H | H | O(CH₂)₂CF₃ | H | CH₃ |
| J148 | CH₃ | H | CH | N | H | F | H | H | CH(CH₃)₂ | H | CH₃ |
| J149 | CH₃ | H | CH | N | H | F | H | H | CH(CH₃)₂ | H | OCH₃ |
| J150 | CH₃ | H | CH | N | H | F | H | H | CH(CH₃)₂ | H | N(CH₃)₂ |
| J151 | CH₃ | H | CH | N | H | F | H | H | O(CH₂)₂CF₃ | H | CH₃ |
| J152 | H | CF₃ | CH | N | H | F | H | H | CH(CH₃)₂ | H | OCH₃ |
| J153 | H | CF₃ | CH | N | H | F | H | H | CH(CH₃)₂ | H | N(CH₃)₂ |
| J154 | H | OCF₃ | CH | N | H | F | H | H | CH(CH₃)₂ | H | OCH₃ |
| J155 | H | OCF₃ | CH | N | H | F | H | H | CH(CH₃)₂ | H | N(CH₃)₂ |
| J156 | H | OCF₃ | CH | N | H | F | H | H | O(CH₂)₂CF₃ | H | CH₃ |
| J157 | OCF₃ | H | CH | N | H | CH₂CH₃ | H | H | CH(CH₃)₂ | H | NHCH₃ |
| J158 | OCF₃ | H | CH | N | H | CH₂CH₃ | H | H | CH(CH₃)₂ | H | NH(CH₂CH₃) |
| J159 | OCF₃ | H | CH | N | H | CH₂CH₃ | H | H | CH(CH₃)₂ | H | NH(CH₂CH₂CH₃) |
| J160 | OCF₃ | H | CH | N | H | CH₂CH₃ | H | H | CH(CH₃)₂ | H | N(CH₂CH₃)₂ |
| J161 | OCF₃ | H | CH | N | H | CH₂CH₃ | H | H | CH(CH₃)₂ | H | N(CH₂CH₂CH₃)₂ |
| J162 | OCF₃ | H | CH | N | H | F | H | H | CH(CH₃)₂ | F | N(CH₃)₂ |
| J163 | OCF₃ | H | CH | N | H | F | H | H | NO₂ | H | CH₃ |
| J164 | OCF₃ | H | CH | N | H | F | H | H | CH(CH₃)₂ | H | CH(CH₃)₂ |
| J165 | OCF₃ | H | CH | N | H | CF₃ | H | H | CH₂OCH₂CF₃ | H | CH₃ |
| J166 | OCF₃ | H | CH | N | H | Cl | H | H | CH(CH₃)₂ | H | N(CH₃)₂ |
| J167 | CF₃ | H | CH | N | H | CH₃ | H | H | CH₂OCH₂CF₃ | H | CH₃ |
| J168 | CF₃ | H | CH | N | H | F | H | H | CH₂OCH₂CF₃ | H | CH₃ |
| J169 | OCF₃ | H | CH | N | H | F | H | H | CH₂CH₂CH₃ | H | OCH₂CH₃ |
| J170 | OCF₃ | H | CH | N | H | OCH₂OCH₃ | H | H | CH(CH₃)₂ | H | CH₃ |
| J171 | OCF₃ | H | CH | N | H | OCH₂OCH₃ | H | H | OCH₂CF₃ | H | CH₃ |
| J172 | OCF₃ | H | CH | N | H | OCH₂OCH₃ | H | H | CH(CH₃)₂ | H | N(CH₃)₂ |
| J173 | OCF₃ | H | CH | N | H | OCH₂OCH₃ | H | H | CH₂OCH₂CF₃ | H | CH₃ |
| J174 | OCF₃ | H | CH | N | H | OCH₂OCH₃ | H | H | CH(CH₃)OCH₂CH₃ | H | CH₃ |
| J175 | OCF₃ | H | CH | N | H | OH | H | H | CH(CH₃)₂ | H | CH₃ |
| J176 | OCF₃ | H | CH | N | H | OH | H | H | OCH₂CF₃ | H | CH₃ |
| J177 | OCF₃ | H | CH | N | H | OH | H | H | CH(CH₃)₂ | H | N(CH₃)₂ |
| J178 | OCF₃ | H | CH | N | H | OH | H | H | CH₂OCH₂CF₃ | H | CH₃ |
| J179 | OCF₃ | H | CH | N | H | OH | H | H | CH(CH₃)OCH₂CH₃ | H | CH₃ |
| J180 | OCF₃ | H | CH | N | H | F | H | H | CH₂OCH₂CH₃ | H | CH₃ |
| J181 | OCF₃ | H | CH | N | H | F | H | H | CH(CF₃)OCH₃ | H | CH₃ |
| J182 | OCF₃ | H | CH | N | H | F | H | H | CF₃ | H | N(CH₃)₂ |
| J183 | CF₃ | H | CH | N | H | F | H | H | CH(CH₃)₂ | F | N(CH₃)₂ |
| J184 | CF₃ | H | CH | N | H | F | H | H | O(CH₂)₂CF₃ | H | N(CH₃)₂ |
| J185 | OCF₃ | H | CH | N | H | F | H | H | CF₃ | H | OCH₃ |
| J186 | SCF₃ | H | CH | N | H | F | H | H | CH₂OCH₂CF₃ | H | CH₃ |
| J187 | SCF₃ | H | CH | N | H | F | H | H | CH(CH₃)OCH₃ | H | CH₃ |
| J188 | OCF₃ | H | CH | N | H | F | H | H | CH(CH₃)₂ | H | N(CH₃)C(═O)CH₃ |
| J189 | OCF₃ | H | CH | N | H | F | H | H | CH(CH₃)₂ | H | N(CH₂CH₃)C(═O)CH₃ |
| J190 | OCF₃ | H | CH | N | H | F | H | H | CF₃ | H | OCH₂CH₃ |
| J191 | OCF₃ | H | CH | N | H | F | H | H | CH(CH₃)₂ | H | OCH₂CH₃ |
| J192 | OCF₃ | H | CH | N | H | F | H | H | OCH₂CH₃ | H | N(CH₃)₂ |
| J193 | OCF₃ | H | CH | N | H | F | H | H | OCH₂CF₃ | H | N(CH₃)₂ |
| J194 | OCF₃ | H | CH | N | H | Cl | H | H | CH₂OCH₂CF₃ | H | CH₃ |
| J195 | SCF₃ | H | CH | N | H | F | H | H | CH₂OCH₂CH₃ | H | CH₃ |
| J196 | OCF₃ | H | CH | N | H | F | H | H | CH₂CH₂CH₃ | H | N(CH₃)₂ |
| J197 | OCF₃ | H | CH | N | H | F | H | H | CH(CH₃)₂ | H | OH |
| J198 | OCF₃ | H | CH | N | H | F | H | H | piperidin-1-yl | H | H |
| | | | | | | | | | Ncyclic(CH₂)₅ | | |
| J199 | OCF₃ | H | CH | N | H | CH₃ | H | H | CH(CH₃)₂ | H | N(CH₃)C(═O)CH₃ |
| J200 | OCF₃ | H | CH | N | H | CH₃ | H | H | CH(CH₃)₂ | H | N(CH₂CH₃)C(═O)CH₃ |
| J201 | OCF₃ | H | CH | N | H | F | H | H | OCH₂CF₃ | H | OCH₃ |
| J202 | OCF₃ | H | CH | N | H | F | H | H | CH(CH₃)₂ | F | OCH₃ |
| J203 | OCF₃ | H | CH | N | H | F | H | H | CH(CH₃)OCH₃ | CH₃ | H |
| J204 | SCF₃ | H | CH | N | H | CH₃ | H | H | CH₂OCH₂CF₃ | H | CH₃ |
| J205 | SCF₃ | H | CH | N | H | CH₃ | H | H | CH₂OCH₂CH₃ | H | CH₃ |
| J206 | OCF₃ | H | CH | N | H | CH₃ | H | H | CH₂OCH₂CH₃ | H | CH₃ |
| J211 | OCF₃ | H | CH | N | H | CHF₂ | H | H | CH(CH₃)₂ | H | CH₃ |
| J212 | OCF₃ | H | CH | N | H | CHF₂ | H | H | OCH₂CF₃ | H | CH₃ |
| J213 | OCF₃ | H | CH | N | H | CHF₂ | H | H | CH(CH₃)₂ | H | N(CH₃)₂ |
| J214 | OCF₃ | H | CH | N | H | CHF₂ | H | H | CH₂OCH₂CH₃ | H | CH₃ |
| J215 | OCF₃ | H | CH | N | H | CHF₂ | H | H | CH₂OCH₂CF₃ | H | CH₃ |
| J216 | OCF₃ | H | CH | N | H | F | H | H | CH₂CH₃ | H | OCH₃ |
| J217 | OCF₃ | H | CH | N | H | Br | H | H | CH(CH₃)₂ | H | CH₃ |
| J218 | OCF₃ | H | CH | N | H | Br | H | H | OCH₂CF₃ | H | CH₃ |
| J219 | OCF₃ | H | CH | N | H | Br | H | H | CH(CH₃)₂ | H | N(CH₃)₂ |
| J220 | OCF₃ | H | CH | N | H | Br | H | H | CH(CH₃)OCH₃ | H | CH₃ |
| J221 | OCF₃ | H | CH | N | H | Br | H | H | CH₂OCH₂CH₃ | H | CH₃ |
| J222 | OCF₃ | H | CH | N | H | Br | H | H | CH₂OCH₂CF₃ | H | CH₃ |
| J223 | SCF₃ | H | CH | N | H | F | H | H | OCH₂CF₃ | H | CH₃ |
| J224 | SCF₃ | H | CH | N | H | F | H | H | OCH₂CH₃ | H | CH₃ |
| J225 | SCF₃ | H | CH | N | H | F | H | H | O(CH₂)3CF₃ | H | CH₃ |
| J226 | SCF₃ | H | CH | N | H | Cl | H | H | CH₂OCH₂CF₃ | H | CH₃ |
| J227 | OCF₃ | H | CH | N | H | CN | H | H | CH(CH₃)₂ | H | CH₃ |
| J228 | OCF₃ | H | CH | N | H | CN | H | H | CH(CH₃)OCH₃ | H | CH₃ |
| J229 | SCF₃ | H | CH | N | H | F | H | H | CH(CF₃)OCH₃ | H | CH₃ |
| J230 | SCF₃ | H | CH | N | H | F | H | H | CH(CH₃)OCH₂CH₃ | H | CH₃ |
| J231 | SCF₃ | H | CH | N | H | Cl | H | H | CH(CH₃)₂ | H | CH₃ |
| J232 | SCF₃ | H | CH | N | H | Cl | H | H | CH(CH₃)OCH₃ | H | CH₃ |
| J233 | SCF₃ | H | CH | N | H | CH₃ | H | H | CH(CH₃)OCH₃ | H | CH₃ |
| J234 | CF₃ | H | CH | N | H | Cl | H | H | CH₂OCH₂CF₃ | H | CH₃ |
| J235 | CF₃ | H | CH | N | H | Cl | H | H | CH₂CH₂CH₃ | H | N(CH₃)₂ |
| J236 | OCF₃ | H | N | N | H | F | H | H | CH(CH₃)OCH₃ | H | CH₃ |
| J237 | OCF₃ | H | N | N | H | F | H | H | CH₂OCH₂CF₃ | H | CH₃ |
| J238 | OCF₃ | H | N | N | H | F | H | H | CH(CH₃)₂ | H | N(CH₃)₂ |
| J239 | OCF₃ | H | N | N | H | F | H | H | CH(CH₃)₂ | H | OCH₃ |
| J240 | OCF₃ | H | N | N | H | CH₃ | H | H | CH(CH₃)OCH₃ | H | CH₃ |
| J241 | OCF₃ | H | N | N | H | CH₃ | H | H | CH₂OCH₂CF₃ | H | CH₃ |
| J242 | OCF₃ | H | CH | N | H | CN | H | H | CH(CH₃)₂ | H | N(CH₃)₂ |
| J243 | OCF₃ | H | CH | N | H | CN | H | H | O(CH₂)₂CF₃ | H | CH₃ |
| J244 | OCF₃ | H | CH | N | H | CN | H | H | CF₃ | H | CH₃ |
| J245 | OCF₃ | H | CH | N | H | CN | H | H | CH₂OCH₂CF₃ | H | CH₃ |
| J246 | OCF₃ | H | CH | N | H | CN | H | H | CH(CH₃)₂ | H | OCH₃ |
| J247 | OCF₃ | H | CH | N | H | CN | H | H | CH₂CH₂CH₃ | H | N(CH₃)₂ |
| J248 | OCF₃ | H | CH | N | H | CN | H | H | CH₂CH₂CH₃ | H | CH₃ |
| J249 | OCF₃ | H | CH | N | H | Cl | H | H | CH₂OCH₂CF₃ | H | CH₃ |
| J250 | OCF₃ | H | N | N | H | Cl | H | H | CH(CH₃)OCH₃ | H | CH₃ |
| J251 | OCF₃ | H | N | N | H | CH₃ | H | H | CH(CH₃)₂ | H | N(CH₃)₂ |
| J252 | OCF₃ | H | N | N | H | CH₃ | H | H | CH(CH₃)₂ | H | OCH₃ |
| J253 | OCF₃ | H | N | N | H | Cl | H | H | CH(CH₃)₂ | H | N(CH₃)₂ |
| J254 | OCF₃ | H | N | N | H | CH₃ | H | H | CH₂CH₂CH₃ | H | N(CH₃)₂ |
| J255 | OCF₃ | H | N | N | H | CH₃ | H | H | O(CH₂)₂CF₃ | H | CH₃ |
| J256 | OCF₃ | H | CH | N | H | F | H | H | CH(CH₃)OCH₃ | H | OCH₃ |
| J257 | OCF₃ | H | N | N | H | F | H | H | O(CH₂)₂CF₃ | H | CH₃ |
| J258 | OCF₃ | H | N | N | H | F | H | H | CH₂CH₂CH₃ | H | N(CH₃)₂ |
| J259 | OCF₃ | H | N | N | H | CN | H | H | CH(CH₃)₂ | H | CH₃ |
| J260 | OCF₃ | H | N | N | H | CF₃ | H | H | CH(CH₃)₂ | H | CH₃ |
| J261 | OCF₃ | H | N | N | H | F | H | H | CH(CH₃)₂ | F | N(CH₃)₂ |
| J262 | OCF₃ | H | N | N | H | F | H | H | CF₃ | H | N(CH₃)₂ |
| J263 | OCF₃ | H | N | N | H | F | H | H | OCH₂CF₃ | H | N(CH₃)₂ |
| J264 | OCF₃ | H | N | N | H | CN | H | H | O(CH₂)₂CF₃ | H | CH₃ |
| J265 | OCF₃ | H | N | N | H | CN | H | H | CH₂OCH₂CF₃ | H | CH₃ |
| J266 | OCF₃ | H | N | N | H | CN | H | H | CH(CH₃)₂ | H | N(CH₃)₂ |
| J267 | OCF₃ | H | N | N | H | CN | H | H | CH(CH₃)OCH₃ | H | CH₃ |
| J268 | OCF₃ | H | N | N | H | CN | H | H | CH(CH₃)₂ | H | OCH₃ |
| J269 | OCF₃ | H | N | N | H | CN | H | H | CH₂CH₂CH₃ | H | N(CH₃)₂ |
| J270 | OCF₃ | H | N | N | H | F | H | H | CH(CH₃)₂ | H | CH₃ |
| J271 | OCF₃ | H | N | N | H | F | H | H | CF₃ | H | CH₃ |
| J272 | OCF₃ | H | N | N | H | F | H | H | CH₂CH₂CH₃ | H | CH₃ |
| J273 | OCF₃ | H | N | N | H | CF₃ | H | H | O(CH₂)₂CF₃ | H | CH₃ |
| J274 | OCF₃ | H | N | N | H | CF₃ | H | H | CH₂OCH₂CF₃ | H | CH₃ |
| J275 | OCF₃ | H | N | N | H | CF₃ | H | H | CH(CH₃)₂ | H | N(CH₃)₂ |
| J276 | OCF₃ | H | N | N | H | CF₃ | H | H | CH(CH₃)OCH₃ | H | CH₃ |
| J277 | OCF₃ | H | N | N | H | CF₃ | H | H | CH(CH₃)₂ | H | OCH₃ |
| J278 | OCF₃ | H | N | N | H | CF₃ | H | H | CH₂CH₂CH₃ | H | N(CH₃)₂ |
| J279 | CF₃ | H | N | N | H | F | H | H | CH(CH₃)₂ | H | CH₃ |
| J280 | CF₃ | H | N | N | H | F | H | H | CH(CH₃)OCH₃ | H | CH₃ |
| J281 | CF₃ | H | N | N | H | F | H | H | CH₂OCH₂CF₃ | H | CH₃ |
| J282 | CF₃ | H | N | N | H | F | H | H | CH₂CH₂CH₃ | H | N(CH₃)₂ |
| J283 | CF₃ | H | N | N | H | F | H | H | CH(CH₃)₂ | H | OCH₃ |
| J284 | CF₃ | H | N | N | H | CH₃ | H | H | CH(CH₃)₂ | H | CH₃ |
| J285 | CF₃ | H | N | N | H | CH₃ | H | H | CH(CH₃)₂ | H | N(CH₃)₂ |
| J286 | CF₃ | H | N | N | H | CH₃ | H | H | CH(CH₃)OCH₃ | H | CH₃ |
| J287 | CN | H | CH | N | H | F | H | H | CH(CH₃)₂ | H | CH₃ |
| J288 | CN | H | CH | N | H | F | H | H | O(CH₂)₂CF₃ | H | CH₃ |
| J289 | CN | H | CH | N | H | F | H | H | CH₂OCH₂CF₃ | H | CH₃ |
| J290 | CN | H | CH | N | H | F | H | H | CH(CH₃)₂ | H | N(CH₃)₂ |
| J291 | CN | H | CH | N | H | F | H | H | CH(CH₃)OCH₃ | H | CH₃ |
| J292 | CF₃ | H | N | N | H | CH₃ | H | H | CH(CH₃)₂ | H | OCH₃ |
| J293 | CF₃ | H | N | N | H | CH₃ | H | H | CH₂CH₂CH₃ | H | CH₃ |
| J294 | CF₃ | H | N | N | H | CH₃ | H | H | CH₂CH₂CH₃ | H | N(CH₃)₂ |
| J295 | CF₃ | H | N | N | H | Cl | H | H | CH(CH₃)₂ | H | CH₃ |
| J296 | CF₃ | H | N | N | H | Cl | H | H | CH(CH₃)₂ | H | N(CH₃)₂ |
| J297 | CF₃ | H | N | N | H | CH₃ | H | H | CH₂OCH₂CF₃ | H | CH₃ |
| J298 | CF₃ | H | N | N | H | F | H | H | CH₂OCH₃ | H | CH₃ |
| J299 | CF₃ | H | N | N | H | F | H | H | CH(CH₃)OCH₃ | CH₃ | H |
| J300 | CF₃ | H | N | N | H | F | H | H | CH₂OCH₂CH₃ | CH₃ | H |
| J301 | CF₃ | H | N | N | H | CF₃ | H | H | CH(CH₃)₂ | H | CH₃ |
| J302 | CF₃ | H | N | N | H | CF₃ | H | H | CH(CH₃)₂ | H | N(CH₃)₂ |
| J303 | CF₃ | H | N | N | H | CF₃ | H | H | CH(CH₃)OCH₃ | H | CH₃ |
| J304 | CF₃ | H | N | N | H | CF₃ | H | H | CH₂OCH₂CF₃ | H | CH₃ |
| J305 | CF₃ | H | N | N | H | CF₃ | H | H | O(CH₂)₂CF₃ | H | CH₃ |
| J306 | CF₃ | H | N | N | H | Cl | H | H | CH(CH₃)OCH₃ | H | CH₃ |
| J307 | CF₃ | H | N | N | H | F | H | H | O(CH₂)₂CF₃ | H | CH₃ |
| J308 | CF₃ | H | N | CH | H | F | H | H | CH₂OCH₂CF₃ | H | CH₃ |
| J309 | CF₃ | H | N | CH | H | F | H | H | CH(CH₃)OCH₃ | H | CH₃ |
| J310 | CF₃ | H | N | CH | H | CH₃ | H | H | CH₂OCH₂CF₃ | H | CH₃ |
| J311 | CF₃ | H | N | CH | H | CH₃ | H | H | CH(CH₃)OCH₃ | H | CH₃ |
| J312 | OCF₃ | H | CH | N | H | F | H | H | CH(CH₃)₂ | H | OCH₃ |
| J313 | OCF₃ | H | CH | N | H | H | H | H | NO₂ | H | CH₃ |
| J314 | OCF₃ | H | CH | N | H | H | H | Cl | CH(CH₃)₂ | H | CH₃ |
| J315 | CF₃ | H | CH | N | H | H | H | H | OCH₂CH₂CF₃ | H | CH₃ |
| J316 | OCF₃ | H | CH | N | H | H | H | H | OCH₂CH₂CF₃ | H | CH₃ |
| J317 | CF₃ | H | CH | N | H | H | H | Cl | OCH₂CH₂CF₃ | H | CH₃ |
| J318 | OCF₃ | H | CH | N | H | F | H | H | CH(CH₃)₂ | H | N(CH₃)₂ |
| J319 | OCF₃ | H | CH | N | H | H | H | H | CH(CH₃)₂ | H | N(CH₃)₂ |
| J320 | OCF₃ | H | CH | N | H | H | H | H | OCH₂CH₂CF₃ | H | OCH₃ |
| J321 | OCF₃ | H | CH | N | H | H | H | H | OCH₂CH₂CF₃ | H | N(CH₃)₂ |
| J322 | OCF₃ | H | CH | N | H | H | H | H | N(CH₃)₂ | H | CH₃ |
| J323 | H | CF₃ | CH | N | H | H | H | H | CH(CH₃)₂ | H | CH₃ |
| J324 | H | CF₃ | CH | N | H | H | H | H | CH(CH₃)₂ | H | OCH₃ |
| J325 | H | CF₃ | CH | N | H | H | H | H | CH(CH₃)₂ | H | N(CH₃)₂ |
| J326 | H | CF₃ | CH | N | H | H | H | H | OCH₂CH₂CF₃ | H | CH₃ |
| J327 | H | OCF₃ | CH | N | H | H | H | H | CH(CH₃)₂ | H | CH₃ |
| J328 | H | OCF₃ | CH | N | H | H | H | H | CH(CH₃)₂ | H | OCH₃ |
| J329 | H | OCF₃ | CH | N | H | H | H | H | CH(CH₃)₂ | H | N(CH₃)₂ |
| J330 | H | OCF₃ | CH | N | H | H | H | H | OCH₂CH₂CF₃ | H | CH₃ |
| J331 | OCF₃ | H | CH | N | H | H | H | H | CH₂OCH₂CF₃ | H | CH₃ |
| J332 | CF₃ | H | CH | N | H | H | H | H | CH₂OCH₂CF₃ | H | CH₃ |
| J333 | SCF₃ | H | CH | N | H | F | H | H | OCH₂CH₂CF₃ | H | CH₃ |
| J334 | SCF₃ | H | CH | N | H | F | H | H | CH(CH₃)₂ | H | N(CH₃)₂ |
| J335 | CF₃ | H | CH | N | H | Cl | H | H | CH(CH₃)OCH₃ | H | CH₃ |
| J336 | CF₃ | H | N | N | H | F | H | H | CH(CH₃)₂ | H | N(CH₃)₂ |
| J337 | CF₃ | H | N | N | H | H | H | H | CH(CH₃)₂ | H | CH₃ |
| J338 | CF₃ | H | N | N | H | H | H | H | CH(CH₃)OCH₃ | H | CH₃ |
| J339 | CF₃ | H | N | N | H | H | H | H | CH(CH₃)₂ | H | N(CH₃)₂ |
| J340 | OCH₂CN | H | CH | N | H | F | H | H | CH(CH₃)OCH₃ | H | CH₃ |
| J341 | CHF₂ | H | N | N | H | F | H | H | CH(CH₃)OCH₃ | H | CH₃ |
| J342 | OCH₂CN | H | CH | N | H | F | H | H | CH(CH₃)₂ | H | N(CH₃)₂ |
| J343 | OCH₂CN | H | CH | N | H | F | H | H | CH₂OCH₂CF₃ | H | CH₃ |
| J344 | OCH₂CN | H | CH | N | H | F | H | H | CH(CH₃)₂ | H | CH₃ |
| J345 | CHF₂ | H | N | N | H | F | H | H | CH₂OCH₃ | H | CH₃ |
| J346 | CHF₂ | H | N | N | H | F | H | H | CH₂OCH₂CF₃ | H | CH₃ |
| J347 | CHF₂ | H | N | N | H | F | H | H | CH(CH₃)₂ | H | N(CH₃)₂ |
| J348 | CF₃ | H | N | CH | H | F | H | H | CH(CH₃)₂ | H | CH₃ |
| J349 | CF₃ | H | N | CH | H | F | H | H | CH(CH₃)₂ | H | N(CH₃)₂ |
| J350 | SCF₃ | H | CH | N | H | H | H | H | CH(CH₃)OCH₃ | H | CH₃ |
| J351 | CHF₂ | H | N | N | H | F | H | H | CH(CH₃)₂ | F | N(CH₃)₂ |
| J352 | CF₃ | H | N | N | H | F | H | H | CH(CH₃)₂ | F | N(CH₃)₂ |
| J353 | CHF₂ | H | N | N | H | F | H | H | CH(OCH₃)CF₃ | H | CH₃ |
| J354 | CHF₂ | H | N | N | H | F | H | H | CH(CH₃)OCH₂CH₃ | H | CH₃ |
| J355 | CHF₂ | H | N | N | H | F | H | H | CH(CH₃)₂ | H | OCH₃ |
| J356 | OCH₂CN | H | CH | N | H | CH₃ | H | H | CH(CH₃)₂ | H | CH₃ |
| J357 | OCH₂CN | H | CH | N | H | CH₃ | H | H | CH(CH₃)₂ | H | N(CH₃)₂ |
| J358 | OCH₂CN | H | CH | N | H | CH₃ | H | H | CH(CH₃)OCH₃ | H | CH₃ |
| J359 | OCH₂CN | H | CH | N | H | CH₃ | H | H | CH₂OCH₂CF₃ | H | CH₃ |
| J360 | OCH₂CN | H | CH | N | H | CH₃ | H | H | OCH₂CH₂CF₃ | H | CH₃ |
| J361 | S(═O)₂CF₃ | H | CH | N | H | F | H | H | CH(CH₃)OCH₃ | H | CH₃ |
| J362 | S(═O)₂CF₃ | H | CH | N | H | CH₃ | H | H | CH(CH₃)OCH₃ | H | CH₃ |
| J363 | S(═O)₂CF₃ | H | CH | N | H | F | H | H | CH₂OCH₂CF₃ | H | CH₃ |
| J364 | S(═O)₂CF₃ | H | CH | N | H | F | H | H | CH(CH₃)₂ | H | N(CH₃)₂ |
| J365 | S(═O)₂CF₃ | H | CH | N | H | CH₃ | H | H | CH₂OCH₂CF₃ | H | CH₃ |
| J366 | S(═O)₂CF₃ | H | CH | N | H | CH₃ | H | H | CH(CH₃)₂ | H | N(CH₃)₂ |
| J367 | SCF₃ | H | CH | N | H | H | H | H | CH₂OCH₂CF₃ | H | CH₃ |
| J368 | SCF₃ | H | CH | N | H | H | H | H | CH(CH₃)₂ | H | N(CH₃)₂ |
| J369 | S(═O)₂CF₃ | H | CH | N | H | F | H | H | OCH₂CH₂CF₃ | H | CH₃ |
| J370 | S(═O)₂CF₃ | H | CH | N | H | F | H | H | CH₂CH₂CH₃ | H | N(CH₃)₂ |
| J371 | S(═O)₂CF₃ | H | CH | N | H | F | H | H | CH(CH₃)₂ | F | N(CH₃)₂ |
| J372 | S(═O)₂CF₃ | H | CH | N | H | F | H | H | OCH₂CH₂CF₃ | H | CN |
| J373 | S(═O)₂CF₃ | H | CH | N | H | CH₃ | H | H | OCH₂CH₂CF₃ | H | CH₃ |
| J374 | S(═O)₂CF₃ | H | CH | N | H | CH₃ | H | H | CH₂CH₂CH₃ | H | N(CH₃)₂ |
| J375 | S(═O)₂CF₃ | H | CH | N | H | CH₃ | H | H | CH(CH₃)₂ | F | N(CH₃)₂ |
| J376 | S(═O)₂CF₃ | H | CH | N | H | CH₃ | H | H | OCH₂CH₂CF₃ | H | CN |
| J377 | CF₃ | H | N | N | H | F | H | H | CH(OCH₃)CF₃ | H | CH₃ |
| J378 | CF₃ | H | N | N | H | F | H | H | CH(CH₃)OCH₂CH₃ | H | CH₃ |
| J379 | S(═O)CF₃ | H | CH | N | H | F | H | H | CH(CH₃)₂ | H | N(CH₃)₂ |
| J380 | S(═O)CF₃ | H | CH | N | H | F | H | H | CH₂OCH₂CF₃ | H | CH₃ |
| J382 | S(═O)CF₃ | H | CH | N | H | F | H | H | OCH₂CH₂CF₃ | H | CH₃ |
| J383 | S(═O)CF₃ | H | CH | N | H | F | H | H | CH₂CH₂CH₃ | H | N(CH₃)₂ |
| J384 | S(═O)CF₃ | H | CH | N | H | F | H | H | CH(CH₃)₂ | F | N(CH₃)₂ |
| J385 | S(═O)CF₃ | H | CH | N | H | F | H | H | OCH₂CH₂CF₃ | H | CN |
| J386 | S(═O)CF₃ | H | CH | N | H | F | H | H | CH(OCH₃)CF₃ | H | CH₃ |
| J387 | SCF₃ | H | CH | N | H | H | H | H | OCH₂CH₂CF₃ | H | CH₃ |
| J388 | SCF₃ | H | CH | N | H | H | H | H | CH₂CH₂CH₃ | H | N(CH₃)₂ |
| J389 | SCF₃ | H | CH | N | H | H | H | H | CH(CH₃)₂ | F | N(CH₃)₂ |
| J392 | SCF₃ | H | CH | N | H | H | H | H | OCH₂CH₂CF₃ | H | CN |
| J395 | OCF₃ | H | CH | N | H | C(═O)CH₃ | H | H | CH(CH₃)₂ | H | CH₃ |
| J396 | OCF₃ | H | CH | N | H | C(═O)CH₃ | H | H | CH(CH₃)₂ | H | N(CH₃)₂ |
| J397 | OCF₃ | H | CH | N | H | C(═O)CH₃ | H | H | CH(CH₃)OCH₃ | H | CH₃ |
| J398 | OCF₃ | H | CH | N | H | C(═O)CH₃ | H | H | CH₂OCH₂CF₃ | H | CH₃ |
| J399 | OCF₃ | H | CH | N | H | C(═O)CH₃ | H | H | OCH₂CH₂CF₃ | H | CH₃ |
| J400 | CF₃ | H | N | N | H | F | H | H | CH₂OCH₂CHF₂ | H | CH₃ |
| J401 | OCF₃ | H | CH | N | H | F | H | H | CH₂OCH₂CHF₂ | H | CH₃ |
| J402 | S(═O)₂CF₃ | H | CH | N | H | CN | H | H | OCH₂CH₂CF₃ | H | CH₃ |
| J411 | S(═O)₂CF₃ | H | CH | N | H | CN | H | H | CH₂OCH₂CF₃ | H | CH₃ |
| J412 | S(═O)₂CF₃ | H | CH | N | H | CN | H | H | OCH₂CH₂CF₃ | H | CN |
| J413 | S(═O)₂CF₃ | H | CH | N | H | CN | H | H | CH(CH₃)₂ | H | CN |
| J414 | S(═O)₂CF₃ | H | CH | N | H | CN | H | H | CH(CH₃)₂ | H | N(CH₃)₂ |
| J415 | CF₃ | H | N | N | H | C(═O)CH₃ | H | H | CH(CH₃)₂ | H | CH₃ |
| J416 | CF₃ | H | N | N | H | C(═O)CH₃ | H | H | CH(CH₃)OCH₃ | H | CH₃ |
| J417 | CF₃ | H | N | N | H | C(═O)CH₃ | H | H | CH(CH₃)₂ | H | N(CH₃)₂ |
| J418 | CF₃ | H | N | N | H | C(═O)CH₃ | H | H | CH₂OCH₂CF₃ | H | CH₃ |
| J419 | CF₃ | H | N | N | H | C(═O)CH₃ | H | H | CH₂CH₂CH₃ | H | N(CH₃)₂ |
| J420 | OCF₃ | H | CH | N | H | CH₃ | H | H | CH₂OCH₂CHF₂ | H | CH₃ |
| J421 | OCF₃ | H | CH | N | H | F | H | H | CH(CH₂CH₃)OCH₃ | H | CH₃ |
| J422 | SF₅ | H | CH | N | H | F | H | H | CH₂OCH₂CF₃ | H | CH₃ |
| J423 | SF₅ | H | CH | N | H | F | H | H | CH(CH₃)OCH₃ | H | CH₃ |
| J425 | CH₂CN | H | CH | N | H | F | H | H | CH₂OCH₂CF₃ | H | CH₃ |
| J436 | OCF₃ | H | CH | N | H | CH₃ | H | H | CH₂OCH₂CF₂Cl | H | CH₃ |
| J437 | OCF₃ | H | CH | N | H | F | H | H | CH₂OCH₂CF₂Cl | H | CH₃ |
| J438 | OCF₃ | H | CH | N | H | F | H | H | CH₂OCH₂CF₃ | CH₃ | H |
| J439 | OCF₃ | H | CH | N | H | F | H | H | CH₂[3-(CF₃)phenyl] | H | CH₃ |
| J440 | OCF₃ | H | CH | N | H | CH₃ | H | H | CH₂[3-(CF₃)phenyl] | H | CH₃ |
| J445 | SF₅ | H | CH | N | H | F | H | H | CH₂OCH₂CH₃ | H | CH₃ |
| J446 | SF₅ | H | CH | N | H | F | H | H | CH₂OCH₂CHF₂ | H | CH₃ |
| J447 | SF₅ | H | CH | N | H | F | H | H | OCH₂CH₂CF₃ | H | CH₃ |
| J448 | OCF₃ | H | CH | N | H | CH₃ | H | H | CH₂OCH₂CF₃ | CH₃ | H |
| J449 | SF₅ | H | CH | N | H | F | H | H | CH₂OCH₂CF₂Cl | H | CH₃ |
| J450 | SF₅ | H | CH | N | H | F | H | H | CH(CH₃)₂ | H | N(CH₃)₂ |
| J451 | CF₃ | H | N | N | H | F | H | H | CH₂OCH₂CF₃ | CH₃ | H |
| J452 | OCF₃ | H | CH | N | H | F | H | H | CH₂OCH₂CCl₃ | H | CH₃ |
| J453 | SF₅ | H | CH | N | H | F | H | H | CH₂OCH₂CCl₃ | H | CH₃ |
| J454 | CF₃ | H | N | N | H | F | H | H | CH₂CH₂CH₃ | H | CH₃ |
| J455 | OCF₃ | H | CH | N | H | F | H | H | CH₂OCH₂CF₃ | CH₃ | CH₃ |
| J456 | OCF₃ | H | CH | N | H | CH₃ | H | H | CH₂phenyl | H | CH₃ |
| J457 | OCF₃ | H | CH | N | H | F | H | H | CH₂phenyl | H | CH₃ |
| J459 | OCF₃ | H | CH | N | H | CH₃ | H | H | CH₂OCH₂CCl₃ | H | CH₃ |
| J460 | OCF₃ | H | CH | N | H | CH₃ | H | H | CH₂OCH₂CF₃ | CH₃ | CH₃ |
| J488 | OCF₃ | H | CH | N | H | CH₃ | H | H | 2-furanyl | H | CH₃ |
| J489 | OCF₃ | H | CH | N | H | F | H | H | 2-furanyl | H | CH₃ |
| J497 | SF₅ | H | CH | N | H | F | H | H | CH₂OCH₂CF₃ | CH₃ | CH₃ |
| J498 | SF₅ | H | CH | N | H | F | H | H | CH₂CH₂CH₃ | H | CH₃ |
| J534 | OCF₃ | H | CH | N | H | CH₃ | H | H | CH₂OCH₂CF₂CH₃ | H | CH₃ |
| J535 | OCF₃ | H | CH | N | H | F | H | H | CH₂OCH₂CF₂CH₃ | H | CH₃ |
| J536 | S(═O)CF₃ | H | CH | N | H | F | H | H | CH(CH₃)OCH₃ | H | CH₃ |
| J539 | SF₅ | H | CH | N | H | F | H | H | CH₂OCH₃ | H | CH₃ |
| J540 | OCF₃ | H | CH | N | H | CH₃ | H | H | CH₂OCH₂CH₂CF₃ | H | CH₃ |
| J541 | SF₅ | H | CH | N | H | F | H | H | OCH₂CH₂CH₃ | H | CH₃ |
| J543 | OCF₃ | H | CH | N | H | F | H | H | CH₂OCH₂CH₂CF₃ | H | CH₃ |
| J544 | SF₅ | H | CH | N | H | F | H | H | CH₂OCH₂CH₂CH₃ | H | CH₃ |
| J545 | OCF₃ | H | CH | N | H | F | H | H | CH₂OCH₂CH₂CH₃ | H | CH₃ |
| J546 | OCF₃ | H | CH | N | H | CH₃ | H | H | CH₂OCH₂CH₂CH₃ | H | CH₃ |
| J547 | CF₃ | H | CH | N | H | F | H | H | CH₂OCH₂CH₂CF₃ | H | CH₃ |
| J548 | SF₅ | H | CH | N | H | CH₃ | H | H | CH₂OCH₂CH₃ | H | CH₃ |
| J549 | SF₅ | H | CH | N | H | CH₃ | H | H | CH₂OCH₂CF₃ | H | CH₃ |
| J550 | CF₃ | H | CH | N | H | F | H | H | CH₂OCH₂CCl₃ | H | CH₃ |
| J551 | OCF₃ | H | CH | N | H | F | H | H | CH₂OCH₂CH₂F | H | CH₃ |
| J552 | SF₅ | H | CH | N | H | F | H | H | CH₂OCH₂CH₂F | H | CH₃ |
| J553 | OCF₃ | H | CH | N | H | F | H | H | CH₂OCH₂CHCl₂ | H | CH₃ |
| J554 | SF₅ | H | CH | N | H | F | H | H | CH₂OCH₂CHCl₂ | H | CH₃ |
| J555 | OCF₃ | H | CH | N | H | CH₃ | H | H | CH₂OCH₂CHCl₂ | H | CH₃ |
| J556 | SF₅ | H | CH | N | H | CH₃ | H | H | CH₂OCH₂CHCl₂ | H | CH₃ |
| J557 | SF₅ | H | CH | N | H | CH₃ | H | H | CH₂OCH₂CH₂F | H | CH₃ |
| J558 | OCF₃ | H | CH | N | H | CH₃ | H | H | CH₂OCH₂CH₂F | H | CH₃ |
| J559 | CF₃ | H | CH | N | H | F | H | H | CH₂OCH₂CHCl₂ | H | CH₃ |
| J560 | OCF₃ | H | CH | N | H | F | H | H | CH₂OCH₂CF₃ | OCH₃ | OCH₃ |
| J561 | SF₅ | H | CH | N | H | F | H | H | CH₂OCH₂CF₃ | OCH₃ | OCH₃ |
| J562 | CF₃ | H | CH | N | H | F | H | H | CH₂phenyl | H | CH₃ |
| J563 | SF₅ | H | CH | N | H | F | H | H | CH₂phenyl | H | CH₃ |
| J564 | OCF₃ | H | CH | N | H | F | H | H | CH₂OCH₂CF₃ | H | OCH₃ |
| J565 | SF₅ | H | CH | N | H | F | H | H | CH₂OCH₂CF₃ | H | OCH₃ |
| J568 | OCF₃ | H | CH | N | H | F | H | H | CH₂OCH₂[3-(F) | H | CH₃ |
| | | | | | | | | | phenyl] | | |
| J569 | SF₅ | H | CH | N | H | CN | H | H | CH₂OCH₂CF₃ | H | CH₃ |
| J570 | OCF₃ | H | CH | N | H | F | H | H | CH₂OCH₂[4-(OCF₃) | H | CH₃ |
| | | | | | | | | | phenyl] | | |
| J571 | OCF₃ | H | CH | N | H | F | H | H | CH₂OCH₂phenyl | H | CH₃ |
| J572 | OCF₃ | H | CH | N | H | F | H | H | CH₂OCH₂[2-(Cl) | H | CH₃ |
| | | | | | | | | | phenyl] | | |
| J573 | OCF₃ | H | CH | N | H | F | H | H | CH₂OCH₂[2,4-di(F) | H | CH₃ |
| | | | | | | | | | phenyl] | | |
| J574 | OCF₃ | H | CH | N | H | F | H | H | CH₂OCH₂[4-(F) | H | CH₃ |
| | | | | | | | | | phenyl] | | |
| J575 | OCF₃ | H | CH | N | H | CH₃ | H | H | CH₂OCH₂CF₃ | H | OCH₃ |
| J577 | SF₅ | H | CH | N | H | CH₃ | H | H | CH₂OCH₂CF₃ | H | OCH₃ |
| J580 | CF₃ | H | CH | N | H | CH₃ | H | H | CH₂OCH₂CF₃ | H | OCH₃ |
| J581 | CF₃ | H | CH | N | H | F | H | H | CH₂OCH₂CF₃ | H | OCH₃ |
| J582 | SF₅ | H | CH | N | H | CN | H | H | CH₂OCH₂CH₃ | H | CH₃ |
| J583 | SF₅ | H | CH | N | H | CN | H | H | CH₂CH₂CH₃ | H | CH₃ |
| J584 | SF₅ | H | CH | N | H | CN | H | H | OCH₂CH₂CF₃ | H | CH₃ |
| J585 | SF₅ | H | CH | N | H | CN | H | H | CH(CH₃)₂ | H | N(CH₃)₂ |
| J586 | SF₅ | H | CH | N | H | CN | H | H | CH(CH₃)OCH₂CH₃ | H | CH₃ |
| J587 | SF₅ | H | CH | N | H | CN | H | H | CH₂OCH₂CF₃ | CH₃ | CH₃ |
| J588 | SF₅ | H | CH | N | H | CN | H | H | CH₂OCH₂CF₃ | OCH₃ | OCH₃ |
| J620 | OCH₂CH₃ | H | CH | N | H | F | H | H | CH₂OCH₂CF₃ | CH₃ | CH₃ |
| J621 | OCF₃ | H | CH | N | H | F | H | H | CH₂OCH₂[4-(F)-2- | H | CH₃ |
| | | | | | | | | | (CF₃)phenyl] | | |
| J622 | OCF₃ | H | CH | N | H | F | H | H | CH₂OCH₂[2,3,4,5,6- | H | CH₃ |
| | | | | | | | | | penta(F)phenyl | | |
| J623 | OCF₃ | H | CH | N | H | F | H | H | CH₂OCH₂[2- | H | CH₃ |
| | | | | | | | | | thiophenyl] | | |
| J624 | OCF₃ | H | CH | N | H | F | H | H | CH₂OCH₂ | H | CH₃ |
| | | | | | | | | | (cyclopropyl) | | |
| J625 | OCH₂CH₃ | H | CH | N | H | F | H | H | CH₂OCH₂CF₃ | H | CH₃ |
| J626 | SF₅ | H | CH | N | H | CF₃ | H | H | CH₂OCH₂CF₃ | H | CH₃ |
| J627 | SF₅ | H | CH | N | H | CF₃ | H | H | CH₂OCH₂CH₃ | H | CH₃ |
| J628 | SF₅ | H | CH | N | H | CF₃ | H | H | CH₂CH₂CH₃ | H | CH₃ |
| J629 | SF₅ | H | CH | N | H | CF₃ | H | H | OCH₂CH₂CF₃ | H | CH₃ |
| J630 | SF₅ | H | CH | N | H | CF₃ | H | H | CH(CH₃)₂ | H | N(CH₃)₂ |
| J631 | SF₅ | H | CH | N | H | CF₃ | H | H | CH(CH₃)OCH₂CH₃ | H | CH₃ |
| J632 | SF₅ | H | CH | N | H | CF₃ | H | H | CH₂OCH₂CF₃ | CH₃ | CH₃ |
| J633 | SF₅ | H | CH | N | H | CF₃ | H | H | CH₂OCH₂CF₃ | OCH₃ | OCH₃ |
| J634 | OCF₃ | H | CH | N | H | F | H | H | CH₂OCH₂CF₃ | H | CH₂CH₃ |
| J635 | OCF₃ | H | CH | N | H | F | H | H | CH₂OCH₂[2-(F) | H | CH₃ |
| | | | | | | | | | phenyl] | | |
| J636 | OCF₃ | H | CH | N | H | F | H | H | CH₂OCH₂(phenyl) | Cl | H |
| J640 | OCF₃ | H | CH | N | H | F | H | H | CH₂OCH₂(2- | H | CH₃ |
| | | | | | | | | | benzthiazolyl) | | |
| J641 | OCF₃ | H | CH | N | H | CH₃ | H | H | CH₂OCH₂CF₃ | H | CH₂CH₃ |
| J642 | CF₃ | H | CH | N | H | F | H | H | CH₂OCH₂CF₃ | H | CH₂CH₃ |
| J643 | OCHF₂ | H | CH | N | H | F | H | H | CH₂OCH₂CF₃ | H | CH₃ |
| J644 | OCHF₂ | H | CH | N | H | F | H | H | CH₂OCH₂CF₃ | H | CH₂CH₃ |
| J653 | OCF₃ | H | CH | N | H | CH₃ | H | H | CH₂OCH₂CF₃ | H | H |
| J659 | OCF₃ | H | CH | N | H | F | H | H | CH₂OCH₂CF₃ | H | H |
| J660 | CF₃ | H | CH | N | H | F | H | H | CH₂OCH₂CF₃ | CH₃ | CH₃ |
| J661 | CF₃ | H | CH | N | H | F | H | H | CH₂OCH₂CF₃ | H | H |
| J662 | OCF₂CF₃ | H | CH | N | H | F | H | H | CH₂OCH₂CF₃ | H | CH₃ |
| J663 | OCF₂CF₃ | H | CH | N | H | F | H | H | CH₂OCH₂CH₃ | H | CH₃ |
| J666 | S(═O)₂CF₃ | H | CH | N | H | F | H | H | CH(OCH₃)CF₃ | H | CH₃ |
| J667 | S(═O)₂CF₃ | H | CH | N | H | F | H | H | CH(CH₃)₂ | H | CH₃ |
| J668 | S(═O)₂CF₃ | H | CH | N | H | F | H | H | CH₂OCH₂CH₂F | H | CH₃ |
| J669 | S(═O)₂CF₃ | H | CH | N | H | F | H | H | CH₂OCH₂CF₃ | H | OCH₃ |
| J670 | S(═O)₂CF₃ | H | CH | N | H | F | H | H | CH₂OCH₂CF₃ | H | F |
| J671 | S(═O)₂CF₃ | H | CH | N | H | F | H | H | CH₂OCH₃ | H | N(CH₃)₂ |
| J672 | S(═O)₂CF₃ | H | CH | N | H | F | H | H | OCH₂CF₃ | H | N(CH₃)₂ |
| J673 | CF₃ | H | CH | N | H | F | H | H | CH₂[4-(F)phenyl] | H | CH₃ |
| J674 | OCF₃ | H | CH | N | H | F | H | H | CH₂[4-(F)phenyl] | H | CH₃ |
| J675 | CF₃ | H | CH | N | H | F | H | H | CH₂[3-(F)phenyl] | H | CH₃ |
| J676 | OCF₃ | H | CH | N | H | F | H | H | CH₂[3-(F)phenyl] | H | CH₃ |
| J681 | OCF₂CF₃ | H | CH | N | H | CH₃ | H | H | CH₂OCH₂CF₃ | H | CH₃ |
| J683 | SF₅ | H | CH | N | H | CN | H | H | CH₂[4-(F)phenyl] | H | CH₃ |
| J684 | CHF₂ | H | CH | N | H | CH₃ | H | H | CH₂OCH₂CF₃ | H | CH₃ |
| J686 | CHF₂ | H | CH | N | H | F | H | H | CH₂OCH₂CF₃ | H | CH₃ |
| J705 | H | OCF₃ | CH | N | H | F | H | H | CH₂OCH₂CF₃ | H | CH₃ |
| J708 | CF₃ | H | CH | N | H | F | H | H | CH₂[2-(F)phenyl] | H | CH₃ |
| J710 | H | CF₃ | CH | N | H | F | H | H | CH₂OCH₂CF₃ | H | CH₃ |
| J711 | H | OCF₃ | CH | N | H | F | H | H | CH₂OCH₂CH₃ | H | CH₃ |
| J712 | OCF₃ | H | CH | N | H | F | H | H | CH₂OCH₂[1-(CH₃)-3- | H | CH₃ |
| | | | | | | | | | (CF₃)thienopyrazolyl] | | |
| J714 | OCF₃ | H | CH | N | H | F | H | H | CH₃ | F | CH₃ |
| J719 | OCF₃ | H | CH | N | H | F | H | H | CH₂OCH₂(phenyl) | H | Cl |
| J720 | OCF₃ | H | CH | N | H | F | H | H | phenyl | H | H |
| J722 | H | OCF₃ | CH | N | H | CH₃ | H | H | CH₂OCH₂CF₃ | H | CH₃ |
| J723 | H | CF₃ | CH | N | H | F | H | H | CH₂OCH₂CH₃ | H | CH₃ |
| J726 | H | OCF₃ | CH | N | H | CH₃ | H | H | CH₂OCH₂CH₃ | H | CH₃ |
| J727 | H | CF₃ | CH | N | H | CH₃ | H | H | CH₂OCH₂CF₃ | H | CH₃ |
| J738 | H | OCF₃ | CH | N | H | F | H | H | CH₂OCH₂CF₃ | CH₃ | CH₃ |
| J740 | OCF₃ | H | CH | N | H | F | H | H | CH₂OCH₂(phenyl) | H | Br |
| J741 | OCF₃ | H | CH | N | H | F | H | H | CH₂OCH₂(phenyl) | H | CF₃ |
| J742 | OCF₃ | H | CH | N | H | F | H | H | CH₂OCH₂(phenyl) | H | H |
| J744 | OCF₃ | H | CH | N | H | F | H | H | H | phenyl | H |
| J745 | OCF₃ | H | CH | N | H | F | H | H | CH₂OCH₂(2- | H | CH₃ |
| | | | | | | | | | benzoxazolyl) | | |
| J746 | CF₃ | H | CH | N | H | F | H | H | CH₂OCH₂[2-(OCF₃) | H | CH₃ |
| | | | | | | | | | phenyl] | | |
| J747 | CF₃ | H | CH | N | H | F | H | H | CH₂OCH₂(phenyl) | H | CH₃ |
| J748 | CF₃ | H | CH | N | H | F | H | H | CH₂OCH₂[3,5-bis | H | CH₃ |
| | | | | | | | | | (CF₃)phenyl] | | |
| J749 | CF₃ | H | CH | N | H | F | H | H | CH₂OCH₂[2-(Cl) | H | CH₃ |
| | | | | | | | | | phenyl] | | |
| J752 | CH₃ | H | CH | N | H | F | H | H | CH₂OCH₂CF₃ | H | CH₃ |
| J753 | CH₃ | H | CH | N | H | F | H | H | CH₂OCH₂CH₃ | H | CH₃ |
| J754 | OCH₃ | H | CH | N | H | F | H | H | CH₂OCH₂CF₃ | H | CH₃ |
| J755 | OCH₃ | H | CH | N | H | F | H | H | CH₂OCH₂CH₃ | H | CH₃ |
| J756 | OCH₃ | H | CH | N | H | F | H | H | CH₂OCH₂CF₃ | CH₃ | CH₃ |
| J757 | OCH₃ | H | CH | N | H | F | H | H | CH₂OCH₂CHF₂ | H | CH₃ |
| J758 | CF₃ | H | CH | N | H | F | H | H | CH₂(pyridin-3-yl) | H | CH₃ |
| J759 | OCF₃ | H | CH | N | H | F | H | H | CH₂(pyridin-3-yl) | H | CH₃ |
| J764 | OCH₂CF₃ | H | CH | N | H | F | H | H | CH₂OCH₂CF₃ | H | CH₃ |
| J766 | OCH₂CF₃ | H | CH | N | H | F | H | H | CH₂OCH₂CH₃ | H | CH₃ |
| J769 | H | H | CH | N | H | F | H | H | CH₂OCH₂CF₃ | H | CH₃ |
| J774 | OCF₃ | H | CH | N | H | F | H | H | CH₂OCH₂[3,5-bis | H | CH₃ |
| | | | | | | | | | (CF₃)phenyl] | | |
| J779 | H | H | CH | N | H | F | H | H | CH₂OCH₂CH₂CF₃ | H | CH₃ |
| J780 | H | H | CH | N | H | CH₃ | H | H | CH₂OCH₂CF₃ | H | CH₃ |
| J781 | H | H | CH | N | H | CH₃ | H | H | CH₂OCH₂CH₂CF₃ | H | CH₃ |
| J782 | F | H | CH | N | H | F | H | H | CH₂OCH₂CF₃ | H | CH₃ |
| J783 | N(CF₃)C(═O) | H | CH | N | H | F | H | H | CH₂CH₂CH₃ | H | CH₃ |
| | OCH₃ | | | | | | | | | | |
| J784 | N(CF₃)C(═O) | H | CH | N | H | CH₃ | H | H | CH₂CH₂CH₃ | H | CH₃ |
| | OCH₃ | | | | | | | | | | |
| J785 | OCF₃ | H | CH | N | H | F | H | H | phenyl | H | CH₃ |
| J793 | CF₂CF₃ | H | CH | N | H | F | H | H | CH₂OCH₂CF₃ | H | CH₃ |
| J794 | CF₂CF₃ | H | CH | N | H | CH₃ | H | H | CH₂OCH₂CF₃ | H | CH₃ |
| J795 | CF₂CF₃ | H | CH | N | H | F | H | H | CH₂OCH₂CH₃ | H | CH₃ |
| J796 | CF₂CF₃ | H | CH | N | H | CH₃ | H | H | CH₂OCH₂CH₃ | H | CH₃ |
| J799 | OCF₃ | H | CH | N | H | F | H | H | CH₃ | H | CF₃ |
| J800 | OCF₃ | H | CH | N | H | H | H | H | NH₂ | H | CH₃ |
| J801 | OCF₂CF₃ | H | CH | N | H | CH₃ | H | H | CH₂OCH₂CH₂F | H | CH₃ |

TABLE 1B

| | | | | | | | | | | (“Formula Four”) |
|---|---|---|---|---|---|---|---|---|---|---|
| 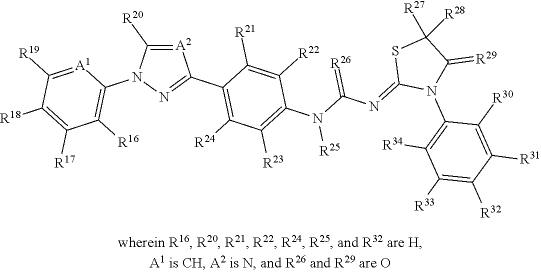 | | | | | | | | | | |
| Cmpd. No. | R¹⁷ | R¹⁸ | R¹⁹ | R²³ | R²⁷ | R²⁸ | R³⁰ | R³¹ | R³³ | R³⁴ |
| J458 | H | OCF₃ | H | F | Cl | Cl | CH₂OCH₂CF₃ | H | CH₃ | H |
| J566 | H | OCF₃ | H | F | H | H | CH₂OCH₂CF₃ | CH₃ | H | H |
| J567 | H | SF₅ | H | F | H | H | CH₂OCH₂CF₃ | CH₃ | H | H |
| J576 | H | OCF₃ | H | CH₃ | H | H | CH₂OCH₂CF₃ | CH₃ | H | H |
| J578 | H | SF₅ | H | CH₃ | H | H | CH₂OCH₂CF₃ | CH₃ | H | H |
| J579 | H | CF₃ | H | CH₃ | H | H | CH₂OCH₂CF₃ | CH₃ | H | H |
| J665 | H | OCF₂CF₃ | H | F | H | H | CH₂OCH₂CF₃ | CH₃ | H | H |
| J677 | H | CF₃ | H | F | H | H | CH₂OCH₂CF₃ | CH₃ | H | H |
| J678 | H | OCF₃ | H | F | H | H | CH₂OCH₂CF₃ | H | CH₃ | CH₃ |
| J679 | H | OCF₃ | H | CH₃ | H | H | CH₂OCH₂CF₃ | H | CH₃ | CH₃ |
| J680 | H | CF₃ | H | F | H | H | CH₂OCH₂CF₃ | H | CH₃ | CH₃ |
| J682 | H | OCF₂CF₃ | H | CH₃ | H | H | CH₂OCH₂CF₃ | CH₃ | H | H |
| J706 | H | H | OCF₃ | F | H | H | CH₂OCH₂CF₃ | CH₃ | H | H |
| J715 | H | OCF₃ | H | F | H | H | H | phenyl | H | H |
| J760 | CF₃ | H | CF₃ | F | H | H | CH₂OCH₂CF₃ | H | CH₃ | H |
| J761 | CF₃ | H | CF₃ | F | H | H | CH₂OCH₂CH₃ | H | CH₃ | H |
| J762 | CF₃ | H | CF₃ | CH₃ | H | H | CH₂OCH₂CF₃ | H | CH₃ | H |
| J763 | CF₃ | H | CF₃ | CH₃ | H | H | CH₂OCH₂CH₃ | H | CH₃ | H |
| J778 | H | OCF₃ | H | F | CH₂OH | CH₂OH | CH₂OCH₂CF₃ | H | CH₃ | H |
| J797 | H | OCF₃ | H | F | H | H | CH₃ | CH₃ | CH₃ | H |
| J798 | H | OCF₃ | H | F | H | H | OCH₃ | CH₃ | H | H |

TABLE 1C

| | | | | | | | | | | (“Formula Five”) |
|---|---|---|---|---|---|---|---|---|---|---|
| 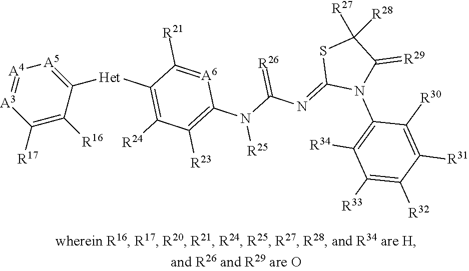 | | | | | | | | | | |
| Cmpd. No. | A³ | A⁴ | A⁵ | Het | A⁶ | R²³ | R³⁰ | R³¹ | R³² | R³³ |
| J381 | CCF₃ | N | N | Het-B | CH | F | CH(CH₃)OCH₃ | H | H | CH₃ |
| J390 | CCF₃ | N | N | Het-B | CH | F | CH₂OCH₂CF₃ | H | H | CH₃ |
| J391 | CCF₃ | N | N | Het-B | CH | F | CH(CH₃)₂ | H | H | N(CH₃)₂ |
| J393 | CCF | N | N | Het-A | CH | F | CH(CH₃)₂ | H | H | N(CH₃)₂ |
| J394 | CCF₃ | N | N | Het-A | CH | F | CH₂OCH₂CF₃ | H | H | CH₃ |
| J403 | COCF₃ | CH | CH | Het-C | CH | H | CH₂OCH₂CF₃ | H | H | CH₃ |
| J404 | COCF₃ | CH | CH | Het-C | CH | H | OCH₂CH₂CF₃ | H | H | CH₃ |
| J405 | COCF₃ | CH | CH | Het-C | CH | H | CH(OCH₃)CF₃ | H | H | CH₃ |
| J406 | COCF₃ | CH | CH | Het-C | CH | H | OCH₂CH₂CF₃ | H | H | CN |
| J407 | COCF₃ | CH | CH | Het-C | CH | H | CH(CH₃)₂ | H | H | CN |
| J408 | COCF₃ | CH | CH | Het-C | CH | H | CH(CH₃)₂ | H | H | N(CH₃)₂ |
| J409 | COCF₃ | CH | CH | Het-C | CH | H | CH₂CH₂CH₃ | H | H | N(CH₃)₂ |
| J410 | COCF₃ | CH | CH | Het-C | CH | H | CH(CH₃)₂ | H | F | N(CH₃)₂ |
| J426 | COCF₃ | CH | CH | Het-D | CH | F | CH(CH₃)OCH₃ | H | H | CH₃ |
| J427 | COCF₃ | CH | CH | Het-E | CH | F | CH₂OCH₂CF₃ | H | H | CH₃ |
| J428 | COCF₃ | CH | CH | Het-E | CH | F | CH(OCH₃)CF₃ | H | H | CH₃ |
| J429 | COCF₃ | CH | CH | Het-F | CH | F | CH(CH₃)OCH₃ | H | H | CH₃ |
| J430 | COCF₃ | CH | CH | Het-F | CH | F | CH₂OCH₂CF₃ | H | H | CH₃ |
| J431 | COCF₃ | CH | CH | Het-F | CH | F | CH(CH₃)₂ | H | H | N(CH₃)₂ |
| J432 | COCF₃ | CH | CH | Het-F | CH | F | CH(CH₃)₂ | H | F | N(CH₃)₂ |
| J433 | COCF₃ | CH | CH | Het-F | CH | F | CH(OCH₃)CF₃ | H | H | CH₃ |
| J434 | COCF₃ | CH | CH | Het-E | CH | F | CH(CH₃)OCH₃ | H | H | CH₃ |
| J435 | COCF₃ | CH | CH | Het-E | CH | F | CH(CH₃)₂ | H | H | N(CH₃)₂ |
| J441 | COCF₃ | CH | CH | Het-C | CH | CH₃ | OCH₂CH₂CF₃ | H | H | CH₃ |
| J442 | COCF₃ | CH | CH | Het-C | CH | CH₃ | CH₂OCH₂CF₃ | H | H | CH₃ |
| J443 | COCF₃ | CH | CH | Het-C | CH | CH₃ | CH(OCH₃)CF₃ | H | H | CH₃ |
| J444 | COCF₃ | CH | CH | Het-C | CH | CH₃ | CH₂OCH₃ | H | H | N(CH₃)₂ |
| J461 | CCF₃ | CH | N | Het-G | CH | F | CH(CH₃)OCH₃ | H | H | CH₃ |
| J462 | COCF₃ | CH | CH | Het-G | CH | CH₃ | CF₃ | H | H | N(CH₃)₂ |
| J463 | COCF₃ | CH | CH | Het-G | CH | H | CH(CH₃)OCH₃ | H | H | CH₃ |
| J465 | COCF₃ | CH | CH | Het-C | CH | CH₃ | OCH₂CH₂CH₂CF₃ | H | H | CH₃ |
| J466 | COCF₃ | CH | CH | Het-C | CH | CH₃ | CH(CH₃)₂ | H | H | CH₃ |
| J467 | COCF₃ | CH | CH | Het-C | CH | CH₃ | CH(CH₃)OCH₃ | H | H | CH₃ |
| J468 | COCF₃ | CH | CH | Het-C | CH | CH₃ | CF₃ | H | H | N(CH₃)₂ |
| J469 | COCF₃ | CH | CH | Het-C | CH | CH₃ | CH(CH₃)₂ | H | H | N(CH₃)₂ |
| J470 | COCF₃ | CH | CH | Het-C | CH | CH₃ | CH(CH₃)₂ | H | F | N(CH₃)₂ |
| J471 | COCF₃ | CH | CH | Het-C | CH | CH₃ | CH₂OCH₂CH₃ | H | H | CH₃ |
| J472 | COCF₃ | CH | CH | Het-C | CH | CH₃ | CH(CH₃)OCH₂CH₃ | H | H | CH₃ |
| J473 | COCF₃ | CH | CH | Het-G | CH | F | CH(CH₃)₂ | H | H | CH₃ |
| J474 | COCF₃ | CH | CH | Het-G | CH | F | CH(CH₃)₂ | H | H | N(CH₃)₂ |
| J476 | COCF₃ | CH | CH | Het-G | CH | F | OCH₂CH₂CF₃ | H | H | CH₃ |
| J477 | COCF₃ | CH | CH | Het-G | CH | CH₃ | CH(CH₃)₂ | H | H | CH₃ |
| J480 | COCF₃ | CH | CH | Het-G | CH | CH₃ | CH(OCH₃)CF₃ | H | H | CH₃ |
| J481 | CCF₃ | CH | N | Het-G | CH | F | CH(CH₃)₂ | H | H | N(CH₃)₂ |
| J482 | CCF₃ | CH | N | Het-G | CH | F | CH(CH₃)₂ | H | H | CH₃ |
| J483 | CCF₃ | CH | N | Het-G | CH | F | CF₃ | H | H | N(CH₃)₂ |
| J484 | CCF₃ | CH | N | Het-G | CH | F | OCH₂CH₂CF₃ | H | H | CH₃ |
| J485 | CCF₃ | CH | N | Het-G | CH | F | CH(OCH₃)CF₃ | H | H | CH₃ |
| J486 | COCF₃ | CH | CH | Het-G | CH | CH₃ | CH(CH₃)OCH₂CH₃ | H | H | CH₃ |
| J487 | CCF₃ | CH | N | Het-G | CH | F | CH₂OCH₂CF₃ | H | H | CH₃ |
| J490 | COCF₃ | CH | CH | Het-H | CH | H | CH₂OCH₂CF₃ | H | H | CH₃ |
| J491 | COCF₃ | CH | CH | Het-H | CH | H | OCH₂CH₂CF₃ | H | H | CH₃ |
| J492 | COCF₃ | CH | CH | Het-H | CH | H | OCH₂CH₂CH₂CF₃ | H | H | CH₃ |
| J493 | COCF₃ | CH | CH | Het-H | CH | H | CH₂OCH₂CH₃ | H | H | CH₃ |
| J494 | COCF₃ | CH | CH | Het-H | CH | H | CH(OCH₃)CF₃ | H | H | CH₃ |
| J495 | COCF₃ | CH | CH | Het-H | CH | H | CF₃ | H | H | N(CH₃)₂ |
| J496 | COCF₃ | CH | CH | Het-H | CH | H | CH₂OCH₃ | H | H | N(CH₃)₂ |
| J499 | COCF₃ | CH | CH | Het-J | CH | F | CH(CH₃)₂ | H | H | CH₃ |
| J500 | COCF₃ | CH | CH | Het-J | CH | F | CH₂OCH₂CF₃ | H | H | CH₃ |
| J501 | COCF₃ | CH | CH | Het-J | CH | F | OCH₂CH₂CF₃ | H | H | CH₃ |
| J502 | COCF₃ | CH | CH | Het-J | CH | F | OCH₂CH₂CH₂CF₃ | H | H | CH₃ |
| J503 | COCF₃ | CH | CH | Het-J | CH | F | CH₂OCH₂CH₃ | H | H | CH₃ |
| J504 | COCF₃ | CH | CH | Het-J | CH | F | CH(CH₃)OCH₃ | H | H | CH₃ |
| J505 | COCF₃ | CH | CH | Het-J | CH | F | CH(OCH₃)CF₃ | H | H | CH₃ |
| J506 | COCF₃ | CH | CH | Het-J | CH | F | CH(CH₃)₂ | H | H | N(CH₃)₂ |
| J507 | COCF₃ | CH | CH | Het-J | CH | F | CH(CH₃)₂ | H | F | N(CH₃)₂ |
| J508 | CCF₃ | CH | N | Het-G | CH | CH₃ | CH(CH₃)OCH₃ | H | H | CH₃ |
| J509 | CCF₃ | CH | N | Het-G | CH | CH₃ | CH(CH₃)₂ | H | H | CH₃ |
| J510 | CCF₃ | CH | N | Het-G | CH | CH₃ | CH(CH₃)₂ | H | H | N(CH₃)₂ |
| J511 | CCF₃ | CH | N | Het-G | CH | CH₃ | CH₂OCH₂CF₃ | H | H | CH₃ |
| J512 | CCF₃ | CH | N | Het-G | CH | CH₃ | OCH₂CH₂CF₃ | H | H | CH₃ |
| J513 | CCF₃ | CH | CH | Het-G | CH | H | CH(CH₃)OCH₃ | H | H | CH₃ |
| J514 | CCF₃ | CH | CH | Het-G | CH | H | CH(CH₃)₂ | H | H | CH₃ |
| J515 | CCF₃ | CH | CH | Het-G | CH | H | CH(CH₃)₂ | H | H | N(CH₃)₂ |
| J516 | CCF₃ | CH | CH | Het-G | CH | H | CH₂OCH₂CF₃ | H | H | CH₃ |
| J517 | CCF₃ | CH | CH | Het-G | CH | H | OCH₂CH₂CF₃ | H | H | CH₃ |
| J518 | CCF₃ | CH | N | Het-G | CH | H | CH(CH₃)OCH₃ | H | H | CH₃ |
| J519 | CCF₃ | CH | N | Het-G | CH | H | CH(CH₃)₂ | H | H | CH₃ |
| J520 | CCF₃ | CH | N | Het-G | CH | H | CH(CH₃)₂ | H | H | N(CH₃)₂ |
| J521 | CCF₃ | CH | N | Het-G | CH | H | CH₂OCH₂CF₃ | H | H | CH₃ |
| J522 | CCF₃ | CH | N | Het-G | CH | H | OCH₂CH₂CF₃ | H | H | CH₃ |
| J523 | CCF₃ | CH | CH | Het-G | CH | CH₃ | CH(CH₃)₂ | H | H | CH₃ |
| J524 | CCF₃ | CH | CH | Het-G | CH | CH₃ | CH(CH₃)₂ | H | H | N(CH₃)₂ |
| J525 | CCF₃ | CH | CH | Het-G | CH | CH₃ | CH(CH₃)OCH₃ | H | H | CH₃ |
| J526 | CCF₃ | CH | CH | Het-G | CH | CH₃ | CH₂OCH₂CF₃ | H | H | CH₃ |
| J527 | CCF₃ | CH | CH | Het-G | CH | CH₃ | OCH₂CH₂CF₃ | H | H | CH₃ |
| J529 | COCF₃ | CH | CH | Het-G | CH | H | CH(CH₃)₂ | H | H | N(CH₃)₂ |
| J532 | CCF₃ | CH | CH | Het-G | CH | F | OCH₂CH₂CF₃ | H | H | CH₃ |
| J533 | COCF₃ | CH | CH | Het-G | CH | CH₃ | CH₂OCH₃ | H | H | N(CH₃)₂ |
| J589 | CSF₅ | CH | CH | Het-G | CH | F | CH(CH₃)₂ | H | H | CH₃ |
| J590 | CSF₅ | CH | CH | Het-G | CH | F | CH₂CH₂CH₃ | H | H | CH₃ |
| J591 | CSF₅ | CH | CH | Het-G | CH | F | CH(CH₃)OCH₃ | H | H | CH₃ |
| J592 | CSF₅ | CH | CH | Het-G | CH | F | CH₂OCH₃ | H | H | CH₃ |
| J593 | CSF₅ | CH | CH | Het-G | CH | F | CH₂OCH₂CH₃ | H | H | CH₃ |
| J594 | CSF₅ | CH | CH | Het-G | CH | F | CH(CH₃)OCH₂CH₃ | H | H | CH₃ |
| J595 | CSF₅ | CH | CH | Het-G | CH | F | OCH₂CH₂CH₃ | H | H | CH₃ |
| J596 | CSF₅ | CH | CH | Het-G | CH | F | OCH₂CF₃ | H | H | CH₃ |
| J597 | CSF₅ | CH | CH | Het-G | CH | F | OCH₂CH₂CF₃ | H | H | CH₃ |
| J598 | CSF₅ | CH | CH | Het-G | CH | F | CH₂OCH₂CF₃ | H | H | CH₃ |
| J599 | CSF₅ | CH | CH | Het-G | CH | F | CH(OCH₃)CF₃ | H | H | CH₃ |
| J600 | CSF₅ | CH | CH | Het-G | CH | F | CF₃ | H | H | N(CH₃)₂ |
| J601 | CSF₅ | CH | CH | Het-G | CH | F | CH₂OCH₃ | H | H | N(CH₃)₂ |
| J602 | CSF₅ | CH | CH | Het-G | CH | F | CH₂CH₂CH₃ | H | H | N(CH₃)₂ |
| J603 | CSF₅ | CH | CH | Het-G | CH | F | CH(CH₃)₂ | H | H | N(CH₃)₂ |
| J604 | CSF₅ | CH | CH | Het-G | CH | F | CH(CH₃)₂ | H | F | N(CH₃)₂ |
| J605 | CSF₅ | CH | CH | Het-G | CH | F | CH(CH₃)₂ | H | H | CN |
| J606 | CSF₅ | CH | CH | Het-G | CH | F | CH(CH₃)OCH₃ | H | H | CN |
| J607 | COCF₃ | CH | CH | Het-F | CH | F | CH(CH₃)₂ | H | H | CH₃ |
| J608 | COCF₃ | CH | CH | Het-F | CH | F | CH₂CH₂CH₃ | H | H | CH₃ |
| J609 | COCF₃ | CH | CH | Het-F | CH | F | CH₂OCH₃ | H | H | CH₃ |
| J610 | COCF₃ | CH | CH | Het-F | CH | F | CH₂OCH₂CH₃ | H | H | CH₃ |
| J611 | COCF₃ | CH | CH | Het-F | CH | F | CF₃ | H | H | N(CH₃)₂ |
| J612 | COCF₃ | CH | CH | Het-F | CH | F | CH₂OCH₃ | H | H | N(CH₃)₂ |
| J613 | CSF₅ | CH | CH | Het-G | CH | CH₃ | CH(CH₃)₂ | H | H | CH₃ |
| J614 | CSF₅ | CH | CH | Het-G | CH | CH₃ | CH(CH₃)OCH₃ | H | H | CH₃ |
| J615 | CSF₅ | CH | CH | Het-G | CH | CH₃ | CH₂OCH₂CH₃ | H | H | CH₃ |
| J616 | CSF₅ | CH | CH | Het-G | CH | CH₃ | CH₂OCH₂CF₃ | H | H | CH₃ |
| J617 | CSF₅ | CH | CH | Het-G | CH | CH₃ | CH(OCH₃)CF₃ | H | H | CH₃ |
| J618 | CSF₅ | CH | CH | Het-G | CH | CH₃ | CH(CH₃)₂ | H | H | N(CH₃)₂ |
| J619 | CSF₅ | CH | CH | Het-G | CH | CH₃ | CH(CH₃)₂ | H | F | N(CH₃)₂ |
| J645 | CS(═O)₂CF₃ | CH | CH | Het-G | CH | F | CH(CH₃)OCH₃ | H | H | CH₃ |
| J646 | CS(═O)₂CF₃ | CH | CH | Het-G | CH | F | CH₂OCH₂CH₃ | H | H | CH₃ |
| J647 | CS(═O)₂CF₃ | CH | CH | Het-G | CH | F | CH₂OCH₂CF₃ | H | H | CH₃ |
| J648 | CS(═O)₂CF₃ | CH | CH | Het-G | CH | F | CH₂OCH₂CHF₂ | H | H | CH₃ |
| J649 | CS(═O)₂CF₃ | CH | CH | Het-G | CH | F | CH(OCH₃)CF₃ | H | H | CH₃ |
| J650 | CS(═O)₂CF₃ | CH | CH | Het-G | CH | F | CH(CH₃)₂ | H | H | N(CH₃)₂ |
| J651 | CS(═O)₂CF₃ | CH | CH | Het-G | CH | F | CH(CH₃)₂ | H | H | CH₃ |
| J652 | CS(═O)₂CF₃ | CH | CH | Het-G | CH | F | CH(CH₃)OCH₃ | H | H | CN |
| J664 | CCF₃ | CH | CH | Het-A | N | CH₃ | CH(CH₃)₂ | H | H | CH₃ |
| J685 | COCF₃ | CH | CH | Het-C | CH | F | CH(CH₃)OCH₃ | H | H | CH₃ |
| J687 | COCF₃ | CH | CH | Het-C | CH | F | CH(OCH₃)CF₃ | H | H | CH₃ |
| J688 | COCF₃ | CH | CH | Het-C | CH | F | OCH₂CF₃ | H | H | N(CH₃)₂ |
| J689 | COCF₃ | CH | CH | Het-C | CH | F | CH₂OCH₂CF₃ | H | H | CH₃ |
| J690 | COCF₃ | CH | CH | Het-C | CH | F | CH₂OCH₂CF₃ | H | H | F |
| J691 | COCF₃ | CH | CH | Het-C | CH | F | CH₂OCH₂CF₃ | H | H | OCH₃ |
| J692 | COCF₃ | CH | CH | Het-C | CH | F | CH₂OCH₂CHF₂ | H | H | CH₃ |
| J693 | COCF₃ | CH | CH | Het-C | CH | F | OCH₂CH₂CF₃ | H | H | CH₃ |
| J694 | CCF₃ | CH | CH | Het-C | CH | F | CH(CH₃)₂ | H | H | CH₃ |
| J695 | CCF₃ | CH | CH | Het-C | CH | F | CH(CH₃)OCH₃ | H | H | CH₃ |
| J696 | CCF₃ | CH | CH | Het-C | CH | F | CH(OCH₃)CF₃ | H | H | CH₃ |
| J697 | CCF₃ | CH | CH | Het-C | CH | F | OCH₂CF₃ | H | H | N(CH₃)₂ |
| J698 | CCF₃ | CH | CH | Het-C | CH | F | CH₂OCH₂CF₃ | H | H | CH₃ |
| J699 | CCF₃ | CH | CH | Het-C | CH | F | CH₂OCH₂CF₃ | H | H | F |
| J700 | CCF₃ | CH | CH | Het-C | CH | F | CH₂OCH₂CF₃ | H | H | OCH₃ |
| J701 | CCF₃ | CH | CH | Het-C | CH | F | CH₂OCH₂CHF₂ | H | H | CH₃ |
| J702 | CCF₃ | CH | CH | Het-C | CH | F | OCH₂CH₂CF₃ | H | H | CH₃ |
| J703 | COCF₃ | CH | CH | Het-C | CH | F | CH₂OCH₂CF₃ | CH₃ | H | H |
| J704 | CCF₃ | CH | CH | Het-C | CH | F | CH₂OCH₂CF₃ | CH₃ | H | H |
| J707 | CCF₃ | CH | CH | Het-A | N | CH₃ | CH₂OCH₂CF₃ | H | H | CH₃ |
| J728 | COCF₃ | CH | CH | Het-C | CH | Cl | CH(CH₃)₂ | H | H | CH₃ |
| J729 | COCF₃ | CH | CH | Het-C | CH | Cl | CH(OCH₃)CF₃ | H | H | CH₃ |
| J730 | COCF₃ | CH | CH | Het-C | CH | Cl | CH₂OCH₃ | H | H | CH₃ |
| J731 | COCF₃ | CH | CH | Het-C | CH | Cl | CH₂OCH₂CF₃ | H | H | CH₃ |
| J732 | COCF₃ | CH | CH | Het-C | CH | Cl | CH₂OCH₂CF₃ | H | H | H |
| J733 | COCF₃ | CH | CH | Het-C | CH | Cl | CH₂OCH₂CF₃ | H | CH₃ | CH₃ |
| J734 | COCF₃ | CH | CH | Het-C | CH | Cl | CH₂OCH₂CF₃ | H | CH₃ | H |
| J735 | COCF₃ | CH | CH | Het-C | CH | Cl | CH₂OCH₂CF₃ | CH₃ | H | H |
| J736 | COCF₃ | CH | CH | Het-C | CH | Cl | CH₂OCH₂CHF₂ | H | H | CH₃ |
| J737 | COCF₃ | CH | CH | Het-C | CH | Cl | OCH₂CH₂CF₃ | H | H | CH₃ |
| J750 | N | COCH₂CF₃ | CH | Het-A | CH | F | CH₂OCH₂CF₃ | H | H | CH₃ |
| J751 | NCH₂CF₃ | C═O | CH | Het-A | CH | F | CH₂OCH₂CF₃ | H | H | CH₃ |
| J765 | COCH₂CF₃ | N | CH | Het-A | CH | F | CH₂OCH₂CF₃ | H | H | CH₃ |
| J767 | COCF₃ | CH | CH | Het-K | CH | H | CH₂OCH₂CF₃ | H | H | CH₃ |
| J768 | COCF₃ | CH | CH | Het-K | CH | H | CH₂OCH₂CHF₂ | H | H | CH₃ |
| J770 | COCF₃ | CH | CH | Het-D | CH | F | CH₂OCH₂CF₃ | H | H | CH₃ |
| J771 | COCF₃ | CH | CH | Het-D | CH | F | CH₂OCH₂CHF₂ | H | H | CH₃ |
| J772 | COCF₃ | CH | CH | Het-D | CH | F | CH₂OCH₃ | H | H | CH₃ |
| J773 | COCF₃ | CH | CH | Het-D | CH | F | CH₂OCH₃ | H | H | N(CH₃)₂ |
| J786 | COCF₃ | CH | CH | Het-L | CH | Cl | CH₂OCH₂CF₃ | H | H | CH₃ |
| J787 | COCF₃ | CH | CH | Het-L | CH | Cl | CH₂OCH₂CHF₂ | H | H | CH₃ |
| J788 | COCF₃ | CH | CH | Het-L | CH | Cl | CH₂OCH₃ | H | H | CH₃ |
| J789 | COCF₃ | CH | CH | Het-L | CH | Cl | CH₂OCH₃ | H | H | N(CH₃)₂ |
| J790 | COCF₃ | CH | CH | Het-L | CH | Cl | OCH₂CH₂CF₃ | H | H | CH₃ |
| J791 | COCF₃ | CH | CH | Het-L | CH | Cl | CH(OCH₃)CF₃ | H | H | CH₃ |
| J792 | COCF₃ | CH | CH | Het-L | CH | Cl | CH(CH₃)₂ | H | H | CH₃ |
| J802 | CH | CH | CH | Het-M | CH | H | CH₂OCH₂CF₃ | H | H | CH₃ |

TABLE 2A
Analytical Data for Compounds in Tables 1A, 1B, and 1C

| | Melting | | |
|---|---|---|---|
| Cmpd. | Point | | |
| No. | (° C.) | MASS SPEC | NMR |
| J1 | | HRMS-ESI (m/z) | ¹H NMR (400 MHz, CDCl₃) δ 8.53 (s, 1H), 8.18 (d, J = 8.5 Hz, |
| | | [M + H]⁺ calcd for | 1H), 8.08-7.93 (m, 2H), 7.79 (d, J = 8.7 Hz, 2H), 7.35 (dd, J = |
| | | C₃₀H₂₇F₃N₆O₃S, | 26.7, 8.3 Hz, 4H), 7.08 (s, 1H), 6.92 (s, 1H), 3.97 (s, 2H), 2.68 |
| | | 609.189; found, | (p, J = 6.8 Hz, 1H), 2.39 (s, 3H), 2.25 (s, 3H), 1.23 (td, J = |
| | | 609.188 | 17.1, 16.7, 7.1 Hz, 6H); ¹⁹F NMR (376 MHz, CDCl₃) δ −58.03 |
| J2 | | ESIMS m/z | ¹H NMR (400 MHz, CDCl₃) δ 8.57 (s, 1H), 8.02 (d, J = 8.5 Hz, |
| | | 659 ([M]⁺) | 1H), 7.80 (d, J = 9.0 Hz, 2H), 7.49 (d, J = 2.2 Hz, 1H), 7.41- |
| | | | 7.36 (m, 4H), 7.33 (d, J = 10.2 Hz, 2H), 6.92 (d, J = 1.8 Hz, |
| | | | 1H), 3.98 (d, J = 3.0 Hz, 2H), 2.73-2.65 (m, 4H), 2.39 (s, 3H), |
| | | | 1.20 (t, J = 6.6 Hz, 6H); ¹⁹F NMR (376 MHz, CDCl₃) δ −85.90, −87.85 |
| J3 | | ESIMS m/z | ¹H NMR (400 MHz, CDCl₃) δ 8.54 (s, 1H), 7.90-7.82 (m, 2H), |
| | | 624 ([M]⁺) | 7.79 (d, J = 9.1 Hz, 2H), 7.66 (d, J = 1.8 Hz, 1H), 7.42-7.34 |
| | | | (m, 4H), 6.94-6.90 (m, 1H), 4.01-3.95 (m, 2H), 3.92 (s, 3H), |
| | | | 2.74-2.62 (m, 1H), 2.39 (s, 3H), 1.73-1.56 (m, 1H), 1.20 |
| | | | (dd, J = 13.7, 6.9 Hz, 6H); ¹⁹F NMR (376 MHz, CDCl₃) δ −58.03 |
| J4 | | ESIMS m/z | ¹H NMR (400 MHz, CDCl₃) δ 8.65 (s, 1H), 8.04 (d, J = 8.5 Hz, |
| | | 592 ([M]⁺) | 1H), 7.93-7.86 (m, 3H), 7.78 (d, J = 8.7 Hz, 3H), 7.46-7.32 |
| | | | (m, 3H), 3.98 (d, J = 3.1 Hz, 2H), 2.70 (s, 3H), 2.39 (s, 3H), |
| | | | 2.37-2.28 (m, 1H), 1.20 (t, J = 6.7 Hz, 6H); ¹⁹F NMR (376 |
| | | | MHz, CDCl₃) δ −62.48 |
| J5 | | ESIMS m/z | ¹H NMR (400 MHz, CDCl₃) δ 8.65 (s, 1H), 8.04 (d, J = 8.5 Hz, |
| | | 608 ([M]⁺) | 1H), 7.92-7.87 (m, 2H), 7.82-7.75 (m, 2H), 7.50 (d, J = 2.2 |
| | | | Hz, 1H), 7.46-7.41 (m, 1H), 7.39 (d, J = 8.8 Hz, 1H), 7.07 (dd, |
| | | | J = 8.7, 2.7 Hz, 1H), 6.63 (d, J = 2.7 Hz, 1H), 3.99 (d, J = 2.7 |
| | | | Hz, 2H), 3.82 (s, 3H), 2.70 (s, 3H), 2.69-2.60 (m, 1H), 1.18 |
| | | | (dd, J = 6.9, 2.9 Hz, 6H); ¹⁹F NMR (376 MHz, CDCl₃) δ −62.48 |
| J6 | | ESIMS m/z | ¹H NMR (400 MHz, CDCl₃) δ 8.54 (s, 1H), 8.47 (d, J = 8.5 Hz, |
| | | 641 ([M]⁺) | 1H), 7.80 (d, J = 9.0 Hz, 2H), 7.66 (d, J = 1.8 Hz, 1H), 7.42- |
| | | | 7.35 (m, 4H), 7.08 (dd, J = 8.7, 2.7 Hz, 1H), 6.63 (d, J = 2.7 Hz, |
| | | | 1H), 3.98 (d, J = 1.6 Hz, 2H), 3.93 (s, 3H), 3.83 (s, 3H), 2.71- |
| | | | 2.58 (m, 1H), 1.18 (dd, J = 8.7, 6.8 Hz, 6H); ¹⁹F NMR (376 |
| | | | MHz, CDCl₃) δ −58.03 |
| J7 | | ESIMS m/z | ¹H NMR (400 MHz, CDCl₃) δ 8.57 (s, 1H), 8.02 (d, J = 8.5 Hz, |
| | | 674 ([M]⁺) | 1H), 7.80 (d, J = 9.0 Hz, 2H), 7.42-7.33 (m, 5H), 7.07 (dd, J = |
| | | | 8.7, 2.7 Hz, 1H), 6.62 (d, J = 2.7 Hz, 1H), 3.98 (d, J = 2.7 Hz, |
| | | | 2H), 3.82 (s, 3H), 2.69 (s, 3H), 2.68-2.59 (m, 1H), 1.22-1.15 |
| | | | (m, 6H); ¹⁹F NMR (376 MHz, CDCl₃) δ −85.90, −87.85 |
| J8 | | HRMS-ESI (m/z) | ¹H NMR (400 MHz, CDCl₃) δ 8.63 (s, 1H), 8.20 (d, J = 8.5 Hz, 1H), 8.05 |
| | | [M + H]⁺ calcd for | (d, J = 8.5 Hz, 1H), 8.00 (s, 1H), 7.91 (d, J = 8.4 Hz, 2H), 7.79 |
| | | C₃₀H₂₇F₃N₆O₃S, | (d, J = 8.5 Hz, 2H), 7.39 (d, J = 8.8 Hz, 1H), 7.13-7.01 (m, 2H), 6.63 |
| | | 609.189; found, | (d, J = 2.7 Hz, 1H), 3.98 (d, J = 0.9 Hz, 2H), 3.83 (s, 3H), 2.65 |
| | | 609.1889 | (p, J = 6.8 Hz, 1H), 2.27 (s, 3H), 1.19 (dd, J = 12.2, 6.8 Hz, 6H); |
| | | | ¹⁹F NMR (376 MHz, CDCl₃) δ −62.25, −62.48, −139.63, −203.03 |
| J9 | | | ¹H NMR (400 MHz, CDCl₃) δ 8.63 (s, 1H), 8.19 (d, J = 8.5 Hz, |
| | | | 1H), 8.05 (d, J = 8.6 Hz, 1H), 8.00 (s, 1H), 7.90 (d, J = 8.4 Hz, |
| | | | 2H), 7.79 (d, J = 8.4 Hz, 2H), 7.42-7.29 (m, 2H), 7.09 (s, 1H), |
| | | | 6.92 (s, 1H), 3.97 (d, J = 1.0 Hz, 2H), 2.69 (p, J = 6.8 Hz, 1H), |
| | | | 2.39 (s, 3H), 2.26 (s, 3H), 1.21 (dd, J = 16.8, 6.8 Hz, 6H); ¹⁹F |
| | | | NMR (376 MHz, CDCl₃) δ −62.25, −62.44 (d, J = 2.7 |
| | | | Hz), −62.48, −62.97, −117.60 |
| J10 | | ESIMS m/z | ¹H NMR (400 MHz, CDCl₃) δ 8.63 (s, 1H), 8.48 (d, J = 8.5 Hz, |
| | | 624 ([M]⁺) | 1H), 7.91 (d, J = 8.3 Hz, 2H), 7.85 (d, J = 4.4 Hz, 1H), 7.79 (d, |
| | | | J = 8.6 Hz, 3H), 7.67 (d, J = 1.8 Hz, 1H), 7.40 (d, J = 8.8 Hz, |
| | | | 1H), 7.08 (dd, J = 8.7, 2.7 Hz, 1H), 6.63 (d, J = 2.7 Hz, 1H), |
| | | | 4.02 (d, J = 18.1 Hz, 2H), 3.94 (s, 3H), 3.83 (s, 3H), 2.71-2.58 |
| | | | (m, 1H), 1.24-1.14 (m, 6H); ¹⁹F NMR (376 MHz, CDCl₃) δ −62.50 |
| J11 | | HRMS-ESI (m/z) | ¹H NMR (400 MHz, CDCl₃) δ 8.63 (s, 1H), 8.49 (t, J = 8.4 Hz, |
| | | [M + H]⁺ calcd for | 1H), 7.98 (dd, J = 8.7, 1.6 Hz, 1H), 7.95-7.85 (m, 2H), 7.80 |
| | | C₂₉H₂₄F₄N₆O₃S, | (d, J = 8.5 Hz, 2H), 7.53 (d, J = 2.9 Hz, 1H), 7.40 (d, J = 8.8 |
| | | 613.1639; found, | Hz, 1H), 7.08 (dd, J = 8.7, 2.7 Hz, 1H), 6.61 (d, J = 2.7 Hz, 1H), |
| | | 613.1661 | 4.17-3.89 (m, 3H), 3.81 (d, J = 17.0 Hz, 3H), 2.62 (h, J = 6.8 |
| | | | Hz, 1H), 1.18 (q, J = 6.1 Hz, 6H); ¹⁹F NMR (376 MHz, CDCl₃) |
| | | | δ −62.52, −130.97 |
| J12 | | ESIMS m/z | ¹H NMR (400 MHz, methanol-d₄) δ 9.13 (s, 1H), 8.01 (d, J = |
| | | 658 ([M]⁺) | 9.1 Hz, 2H), 7.87 (d, J = 8.4 Hz, 1H), 7.59-7.43 (m, 5H), 7.29 |
| | | | (d, J = 7.9 Hz, 1H), 7.27-7.21 (m, 1H), 7.04 (d, J = 1.8 Hz, |
| | | | 1H), 4.09 (d, J = 6.0 Hz, 2H), 2.64 (s, 3H), 2.45-2.38 (m, 2H), |
| | | | 2.37 (s, 3H), 1.66-1.48 (m, 2H), 0.93 (t, J = 7.4 Hz, 3H); ¹⁹F |
| | | | NMR (376 MHz, CDCl₃) δ −85.90, −87.85 |
| J13 | | ESIMS m/z | ¹H NMR (400 MHz, CDCl₃) δ 8.63 (s, 1H), 8.48 (d, J = 8.4 Hz, |
| | | 609 ([M]⁺) | 1H), 7.91 (d, J = 8.4 Hz, 2H), 7.86 (s, 1H), 7.85-7.82 (m, 1H), |
| | | | 7.79 (d, J = 8.6 Hz, 2H), 7.67 (d, J = 1.7 Hz, 1H), 7.39 (d, J = |
| | | | 8.1 Hz, 1H), 6.92 (s, 1H), 3.97 (d, J = 1.6 Hz, 2H), 3.94 (s, 3H), |
| | | | 2.74-2.63 (m, 1H), 2.40 (s, 3H), 2.01 (s, 1H), 1.20 (dd, J = |
| | | | 13.7, 6.8 Hz, 6H); ¹⁹F NMR (376 MHz, CDCl₃) δ −62.49 |
| J14 | | ESIMS m/z | ¹H NMR (400 MHz, CDCl₃) δ 8.57 (s, 1H), 8.07-7.97 (m, 2H), |
| | | 645 ([M]⁺) | 7.80 (d, J = 9.0 Hz, 2H), 7.50 (d, J = 2.2 Hz, 1H), 7.42 (dd, J = |
| | | | 8.5, 2.3 Hz, 1H), 7.38 (d, J = 8.9 Hz, 2H), 7.34-7.28 (m, 2H), |
| | | | 6.94 (s, 1H), 3.97 (d, J = 2.1 Hz, 2H), 2.96 (s, 3H), 2.88 (d, J = |
| | | | 0.6 Hz, 3H), 2.44 (t, J = 7.6 Hz, 2H), 1.19 (t, J = 7.6 Hz, 3H); |
| | | | ¹⁹F NMR (376 MHz, CDCl₃) δ −85.90, −87.85 |
| J15 | | ESIMS m/z | ¹H NMR (400 MHz, CDCl₃) δ 8.63 (s, 1H), 8.20 (d, J = 8.5 Hz, |
| | | 593 ([M]⁺) | 1H), 8.05 (d, J = 8.4 Hz, 1H), 8.00 (s, 1H), 7.90 (d, J = 8.6 Hz, |
| | | | 2H), 7.79 (d, J = 8.5 Hz, 2H), 7.34-7.27 (m, 1H), 7.14-7.08 |
| | | | (m, 1H), 6.99-6.91 (m, 1H), 3.97 (d, J = 1.8 Hz, 2H), 3.93 (s, |
| | | | 1H), 2.39 (s, 5H), 2.27 (s, 3H), 1.60 (q, J = 7.6 Hz, 2H), 0.93 (t, |
| | | | J = 7.3 Hz, 3H); ¹⁹F NMR (376 MHz, CDCl₃) δ −62.49 |
| J16 | 140 | ESIMS m/z | ¹H NMR (400 MHz, CDCl₃) δ 8.63 (s, 1H), 8.20 (d, J = 8.5 Hz, |
| | | 579 ([M + H]⁺) | 1H), 8.04 (d, J = 8.6 Hz, 1H), 8.00 (s, 1H), 7.90 (d, J = 8.4 Hz, |
| | | | 2H), 7.79 (d, J = 8.5 Hz, 2H), 7.31 (t, J = 9.5 Hz, 2H), 7.10 (s, |
| | | | 1H), 6.95 (s, 1H), 3.97 (d, J = 1.3 Hz, 2H), 2.46 (dd, J = 15.3, |
| | | | 7.7 Hz, 2H), 2.41 (d, J = 6.1 Hz, 3H), 2.27 (s, 3H), 1.20 (t, J = |
| | | | 7.6 Hz, 3H); ¹⁹F NMR (376 MHz, CDCl₃) δ −62.48 |
| J18 | | ESIMS m/z | ¹H NMR (400 MHz, CDCl₃) δ 8.63 (s, 1H), 8.20 (d, J = 8.5 Hz, |
| | | 609 ([M − 15]⁺) | 1H), 8.05 (d, J = 9.2 Hz, 1H), 8.00 (s, 1H), 7.91 (d, J = 8.6 Hz, |
| | | | 2H), 7.79 (d, J = 8.6 Hz, 2H), 7.39 (d, J = 8.8 Hz, 1H), 7.12 (s, |
| | | | 1H), 7.07 (dd, J = 8.8, 2.7 Hz, 1H), 6.63 (d, J = 2.7 Hz, 1H), |
| | | | 3.98 (d, J = 1.0 Hz, 2H), 3.83 (s, 3H), 2.72-2.58 (m, 1H), 2.27 |
| | | | (s, 3H), 1.19 (dd, J = 12.2, 6.9 Hz, 6H); ¹⁹F NMR (376 MHz, |
| | | | CDCl₃) δ −62.48 |
| J19 | | HRMS-ESI (m/z) | ¹H NMR (400 MHz, CDCl₃) δ 8.54 (s, 1H), 8.52-8.40 (m, 1H), |
| | | [M + H]⁺ calcd for | 8.03-7.92 (m, 1H), 7.88 (dd, J = 12.0, 1.8 Hz, 1H), 7.84- |
| | | C₂₉H₂₄F₄N₆O₃S, | 7.71 (m, 2H), 7.52 (d, J = 3.1 Hz, 1H), 7.45-7.29 (m, 4H), |
| | | 613.1639; found, | 6.96-6.82 (m, 1H), 3.99 (d, J = 2.3 Hz, 2H), 2.66 (hept, J = |
| | | 613.1642 | 6.9 Hz, 1H), 2.39 (s, 3H), 1.20 (dd, J = 9.2, 6.8 Hz, 6H); ¹⁹F |
| | | | NMR (376 MHz, CDCl₃) δ −58.03, −131.03, −135.37 |
| J20 | | ESIMS m/z | ¹H NMR (400 MHz, CDCl₃) δ 8.63 (s, 1H), 8.50 (t, J = 8.3 Hz, |
| | | 597 ([M − 15]⁺) | 1H), 7.98 (d, J = 8.7 Hz, 1H), 7.90 (d, J = 9.0 Hz, 3H), 7.80 (d, |
| | | | J = 8.5 Hz, 2H), 7.55 (s, 1H), 7.29 (d, J = 7.0 Hz, 2H), 6.93 (s, |
| | | | 1H), 3.99 (d, J = 2.5 Hz, 2H), 2.40 (s, 3H), 2.37 (d, J = 8.0 Hz, |
| | | | 2H), 1.64-1.56 (m, 2H), 0.92 (t, J = 7.3 Hz, 3H); ¹⁹F NMR |
| | | | (376 MHz, CDCl₃) δ −62.51, −130.98 |
| J21 | | ESIMS m/z | ¹H NMR (400 MHz, CDCl₃) δ 8.62 (s, 1H), 8.14 (d, J = 8.7 Hz, |
| | | 678 ([M]⁺) | 2H), 7.94-7.88 (m, 2H), 7.78 (d, J = 8.6 Hz, 3H), 7.62 (d, J = |
| | | | 8.7 Hz, 2H), 7.39 (d, J = 8.3 Hz, 2H), 6.94-6.89 (m, 1H), 3.98 |
| | | | (d, J = 3.1 Hz, 2H), 2.75-2.62 (m, 1H), 2.39 (s, 3H), 1.20 (t, J = |
| | | | 6.8 Hz, 6H); ¹⁹F NMR (376 MHz, CDCl₃) δ −75.62, −182.17 |
| J22 | | ESIMS m/z | ¹H NMR (400 MHz, CDCl₃) δ 8.56 (s, 1H), 8.02 (d, J = 8.5 Hz, |
| | | 624 ([M]⁺) | 1H), 7.79 (d, J = 9.0 Hz, 2H), 7.49 (d, J = 2.3 Hz, 1H), 7.42- |
| | | | 7.35 (m, 4H), 7.32 (s, 1H), 7.07 (dd, J = 8.8, 2.8 Hz, 1H), 6.62 |
| | | | (d, J = 2.7 Hz, 1H), 3.98 (d, J = 2.7 Hz, 2H), 3.82 (s, 3H), 2.69 |
| | | | (s, 3H), 2.68-2.59 (m, 1H), 1.18 (dd, J = 6.8, 2.9 Hz, 6H); ¹⁹F |
| | | | NMR (376 MHz, CDCl₃) δ −58.04 |
| J23 | | ESIMS m/z | ¹H NMR (400 MHz, CDCl₃) δ 8.57 (s, 1H), 8.02 (d, J = 8.4 Hz, |
| | | 608 ([M]⁺) | 1H), 7.79 (d, J = 9.0 Hz, 2H), 7.49 (d, J = 2.2 Hz, 1H), 7.38 (dd, |
| | | | J = 8.3, 4.2 Hz, 4H), 7.35-7.31 (m, 2H), 6.94-6.89 (m, 1H), |
| | | | 3.98 (d, J = 3.0 Hz, 2H), 2.69 (s, 4H), 2.39 (s, 3H), 1.19 (t, J = |
| | | | 6.7 Hz, 6H); ¹⁹F NMR (376 MHz, CDCl₃) δ −58.04 |
| J24 | | ESIMS m/z | ¹H NMR (400 MHz, CDCl₃) δ 8.54 (s, 1H), 8.18 (d, J = 8.5 Hz, |
| | | 659 ([M]⁺) | 1H), 8.03 (dd, J = 8.5, 2.2 Hz, 2H), 7.98 (d, J = 1.7 Hz, 1H), |
| | | | 7.80 (d, J = 9.0 Hz, 2H), 7.39 (d, J = 9.2 Hz, 2H), 7.35-7.29 |
| | | | (m, 1H), 7.14 (s, 1H), 6.92 (s, 1H), 3.97 (d, J = 1.0 Hz, 2H), |
| | | | 2.73-2.63 (m, 1H), 2.39 (s, 3H), 2.25 (s, 3H), 1.30-1.12 (m, |
| | | | 6H); ¹⁹F NMR (376 MHz, CDCl₃) δ −85.90, −87.85 |
| J25 | | ESIMS m/z | ¹H NMR (400 MHz, CDCl₃) δ 8.54 (s, 1H), 8.19 (d, J = 8.5 Hz, |
| | | 675 ([M + H]⁺) | 1H), 8.04 (dd, J = 8.5, 2.1 Hz, 1H), 7.99 (s, 1H), 7.80 (d, J = |
| | | | 9.0 Hz, 2H), 7.39 (dd, J = 8.8, 1.9 Hz, 3H), 7.15 (s, 1H), 7.07 |
| | | | (dd, J = 8.7, 2.7 Hz, 1H), 6.63 (d, J = 2.7 Hz, 1H), 3.98 (d, J = |
| | | | 0.9 Hz, 2H), 3.82 (s, 3H), 2.72-2.56 (m, 1H), 2.26 (s, 3H), |
| | | | 1.19 (dd, J = 12.2, 6.9 Hz, 6H); ¹⁹F NMR (376 MHz, CDCl₃) |
| | | | δ −85.90, −87.85 |
| J26 | | HRMS-ESI (m/z) | ¹H NMR (400 MHz, CDCl₃) δ 8.54 (s, 1H), 8.48 (t, J = 8.3 Hz, |
| | | [M + H]⁺ calcd for | 1H), 8.06-7.93 (m, 1H), 7.88 (dd, J = 12.0, 1.9 Hz, 1H), 7.83- |
| | | C₃₀H₂₄F₆N₆O₃S, | 7.73 (m, 2H), 7.53 (d, J = 3.1 Hz, 1H), 7.36 (ddd, J = 25.2, |
| | | 663.1608; found, | 8.5, 2.0 Hz, 4H), 6.90 (t, J = 1.2 Hz, 1H), 3.99 (d, J = 2.3 Hz, |
| | | 663.1608 | 2H), 2.65 (h, J = 6.9 Hz, 1H), 2.39 (s, 3H), 1.26-1.12 (m, 6H); |
| | | | ¹⁹F NMR (376 MHz, CDCl₃) δ −85.90, −87.85, −131.02 |
| J27 | | HRMS-ESI (m/z) | ¹H NMR (400 MHz, CDCl₃) δ 8.55 (s, 1H), 8.48 (t, J = 8.4 Hz, |
| | | [M + H]⁺ calcd for | 1H), 7.97 (dd, J = 8.4, 1.3 Hz, 1H), 7.89 (dd, J = 11.9, 1.9 Hz, |
| | | C₃₀H₂₄F₆N₆O₄S, | 1H), 7.83-7.71 (m, 2H), 7.56 (d, J = 3.0 Hz, 1H), 7.39 (dd, J = |
| | | 679.1557; found, | 8.9, 2.2 Hz, 3H), 7.08 (dd, J = 8.7, 2.7 Hz, 1H), 6.62 (d, J = 2.7 |
| | | 679.1568 | Hz, 1H), 4.00 (d, J = 2.1 Hz, 2H), 3.83 (s, 3H), 2.63 (hept, J = |
| | | | 6.8 Hz, 1H), 1.18 (t, J = 6.5 Hz, 6H); ¹⁹F NMR (376 MHz, |
| | | | CDCl₃) δ −85.90, −87.85, −130.98 |
| J28 | 150 | ESIMS m/z | ¹H NMR (400 MHz, CDCl₃) δ 8.54 (s, 1H), 8.47 (d, J = 8.5 Hz, |
| | | 674 ([M]⁺) | 1H), 7.81 (d, J = 9.0 Hz, 3H), 7.66 (d, J = 1.8 Hz, 1H), 7.39 (d, |
| | | | J = 8.4 Hz, 4H), 7.33 (d, J = 8.9 Hz, 1H), 6.92 (s, 1H), 3.97 (d, |
| | | | J = 1.7 Hz, 2H), 3.93 (s, 3H), 2.73-2.62 (m, 1H), 2.40 (s, 3H), |
| | | | 1.20 (dd, J = 13.4, 6.8 Hz, 6H); ¹⁹F NMR (376 MHz, CDCl₃) |
| | | | δ −85.89, −87.85 |
| J29 | 138 | ESIMS m/z | ¹H NMR (400 MHz, CDCl₃) δ 8.54 (s, 1H), 8.47 (d, J = 8.5 Hz, |
| | | 691 ([M]⁺) | 1H), 7.87-7.75 (m, 4H), 7.67 (d, J = 1.8 Hz, 1H), 7.40 (dd, J = |
| | | | 8.9, 3.0 Hz, 3H), 7.08 (dd, J = 8.7, 2.7 Hz, 1H), 6.63 (d, J = 2.7 |
| | | | Hz, 1H), 4.02-3.96 (m, 2H), 3.94 (s, 3H), 3.83 (s, 3H), 2.64 |
| | | | (q, J = 6.9 Hz, 1H), 1.19 (dd, J = 8.6, 6.8 Hz, 6H); ¹⁹F NMR |
| | | | (376 MHz, CDCl₃) δ −85.89, −87.85 |
| J30 | | HRMS-ESI (m/z) | ¹H NMR (400 MHz, CDCl₃) δ 8.62 (s, 1H), 8.58-8.37 (m, 1H), |
| | | [M + H]⁺ calcd for | 8.03-7.85 (m, 2H), 7.83-7.68 (m, 3H), 7.47 (d, J = 9.1 Hz, |
| | | C₂₇H₁₇F₇N₆O₃S, | 2H), 7.42-7.28 (m, 2H), 7.12 (s, 1H), 4.07-3.83 (m, 2H), |
| | | 639.1044; found | 2.49 (d, J = 13.4 Hz, 3H); ¹⁹F NMR (376 MHz, CDCl₃) δ −58.03, |
| | | 639.1047 | −61.12, −131.52, −135.36 |
| J31 | | HRMS-ESI (m/z) | ¹H NMR (400 MHz, CDCl₃) δ 8.54 (s, 1H), 8.49 (t, J = 8.3 Hz, |
| | | [M + H]⁺ calcd for | 1H), 7.97 (d, J = 8.8 Hz, 1H), 7.88 (dd, J = 12.1, 1.8 Hz, 1H), |
| | | C₂₈H₁₉F₇N₆O₄S, | 7.84-7.73 (m, 2H), 7.50 (d, J = 3.0 Hz, 1H), 7.44-7.29 (m, |
| | | 669.1149; found, | 2H), 7.14-7.03 (m, 1H), 6.97 (dd, J = 18.0, 8.4 Hz, 1H), 4.35 |
| | | 669.1152 | (dtdd, J = 11.6, 8.1, 6.9, 3.6 Hz, 2H), 4.03-3.85 (m, 2H), 2.75 |
| | | | (s, 1H), 2.40 (s, 3H); ¹⁹F NMR (376 MHz, CDCl₃) |
| | | | δ −58.03, −74.17, −131.57, −135.37 |
| J32 | | HRMS-ESI (m/z) | ¹H NMR (400 MHz, CDCl₃) δ 8.54 (s, 1H), 8.44 (td, J = 8.3, 3.0 |
| | | [M + H]⁺ calcd for | Hz, 1H), 7.98 (dd, J = 8.7, 2.0 Hz, 1H), 7.94-7.85 (m, 1H), |
| | | C₂₉H₂₃ClF₄N₆O₃S, | 7.83-7.73 (m, 2H), 7.62 (d, J = 3.0 Hz, 1H), 7.48-7.32 (m, |
| | | 647.125; found, | 4H), 7.01-6.88 (m, 1H), 5.79 (d, J = 17.4 Hz, 1H), 2.70 (dp, J = |
| | | 647.1241 | 72.2, 6.9 Hz, 1H), 2.40 (d, J = 6.4 Hz, 3H), 1.20 (ddd, J = |
| | | | 13.4, 10.5, 6.9 Hz, 6H); ¹⁹F NMR (376 MHz, CDCl₃) |
| | | | δ −58.02, −130.71 |
| J33 | | HRMS-ESI (m/z) | ¹H NMR (400 MHz, CDCl₃) δ 8.54 (s, 1H), 8.45 (t, J = 8.4 Hz, |
| | | [M + H]⁺ calcd for | 1H), 7.98 (d, J = 8.6 Hz, 1H), 7.90 (dd, J = 12.0, 1.9 Hz, 1H), |
| | | C₂₈H₁₈ClF₇N₆O₄S, | 7.83-7.73 (m, 2H), 7.59 (s, 1H), 7.42-7.29 (m, 3H), 7.11 |
| | | 703.076; found, | (dd, J = 23.1, 2.2 Hz, 1H), 6.99 (dd, J = 8.5, 2.4 Hz, 1H), 5.77 |
| | | 703.0754 | (d, J = 16.9 Hz, 1H), 4.47-4.28 (m, 2H), 2.41 (d, J = 6.4 Hz, |
| | | | 3H); ¹⁹F NMR (376 MHz, CDCl₃) δ −58.03, −73.86, −131.29 |
| J34 | | HRMS-ESI (m/z) | ¹H NMR (400 MHz, CDCl₃) δ 8.55 (s, 1H), 8.44 (td, J = 8.3, 1.5 |
| | | [M + H]⁺ calcd for | Hz, 1H), 8.05-7.94 (m, 1H), 7.89 (dd, J = 12.0, 1.9 Hz, 1H), |
| | | C₂₇H₁₆ClF₇N₆O₃S, | 7.84-7.72 (m, 3H), 7.57 (d, J = 3.2 Hz, 1H), 7.49 (d, J = 8.2 |
| | | 673.0654; found | Hz, 1H), 7.43-7.34 (m, 2H), 7.23-7.09 (m, 1H), 5.80 (d, J = |
| | | 673.0648 | 35.6 Hz, 1H), 2.57-2.43 (m, 3H); ¹⁹F NMR (376 MHz, CDCl₃) |
| | | | δ −58.03, −60.93, −131.17 |
| J35 | | HRMS-ESI (m/z) | ¹H NMR (400 MHz, CDCl₃) δ 8.58-8.45 (m, 2H), 8.03-7.82 |
| | | [M + H]⁺ calcd for | (m, 2H), 7.84-7.70 (m, 2H), 7.51 (d, J = 3.0 Hz, 1H), 7.45- |
| | | C₂₉H₂₁F₇N₆O₄S, | 7.33 (m, 2H), 7.10-6.86 (m, 3H), 4.22 (tq, J = 6.7, 3.8 Hz, |
| | | 683.1306; found, | 2H), 3.97 (dd, J = 32.2, 1.4 Hz, 2H), 2.65-2.46 (m, 2H), 2.36 |
| | | 683.1306 | (d, J = 17.5 Hz, 3H); ¹⁹F NMR (376 MHz, CDCl₃) |
| | | | δ −58.03, −64.61, −131.48 |
| J36 | | HRMS-ESI (m/z) | ¹H NMR (400 MHz, CDCl₃) δ 8.54 (s, 1H), 8.46 (t, J = 8.3 Hz, |
| | | [M + H]⁺ calcd for | 1H), 7.98 (d, J = 8.6 Hz, 1H), 7.90 (dt, J = 12.0, 1.6 Hz, 1H), |
| | | C₂₉H₂₀ClF₇N₆O₄S, | 7.83-7.74 (m, 2H), 7.60 (d, J = 3.4 Hz, 1H), 7.35 (dd, J = |
| | | 717.0916; found, | 30.8, 8.4 Hz, 3H), 7.14-7.03 (m, 1H), 6.98 (dd, J = 8.5, 5.5 |
| | | 717.0918 | Hz, 1H), 5.76 (d, J = 1.2 Hz, 1H), 4.35-4.14 (m, 2H), 2.58 |
| | | | (tddd, J = 24.4, 15.8, 12.7, 8.1 Hz, 2H), 2.39 (d, J = 5.0 Hz, |
| | | | 3H); ¹⁹F NMR (376 MHz, CDCl₃) δ −58.03, −64.70, −131.21 |
| J37 | | HRMS-ESI (m/z) | ¹H NMR (400 MHz, CDCl₃) δ 8.52 (d, J = 15.7 Hz, 2H), 8.19 (d, |
| | | [M + H]⁺ calcd for | J = 1.9 Hz, 1H), 8.09 (d, J = 8.9 Hz, 1H), 7.85-7.69 (m, 2H), |
| | | C₂₉H₂₄ClF₃N₆O₄S, | 7.44-7.33 (m, 2H), 7.06 (ddd, J = 11.5, 8.7, 2.7 Hz, 1H), 6.69- |
| | | 645.1293; found, | 6.50 (m, 1H), 4.14-3.90 (m, 2H), 3.81 (d, J = 10.2 Hz, 3H), |
| | | 645.1295 | 2.64 (p, J = 6.9 Hz, 1H), 1.28-1.12 (m, 6H); ¹⁹F NMR (376 |
| | | | MHz, CDCl₃) δ −58.03 |
| J38 | | HRMS-ESI (m/z) | ¹H NMR (400 MHz, CDCl₃) δ 8.58-8.46 (m, 2H), 8.18 (d, J = |
| | | [M + H]⁺ calcd for | 1.9 Hz, 1H), 8.15-8.03 (m, 1H), 7.83-7.69 (m, 2H), 7.46- |
| | | C₂₉H₂₄ClF₃N₆O₃S, | 7.28 (m, 4H), 6.95-6.78 (m, 1H), 4.16-3.91 (m, 2H), 2.68 (h, |
| | | 629.1344; found, | J = 7.4, 6.9 Hz, 1H), 2.37 (d, J = 12.8 Hz, 3H), 1.28-1.12 (m, |
| | | 629.1345 | 7H); ¹⁹F NMR (376 MHz, CDCl₃) δ −58.02 |
| J39 | | HRMS-ESI (m/z) | ¹H NMR (400 MHz, CDCl₃) δ 8.53 (d, J = 7.6 Hz, 2H), 8.18 (d, |
| | | [M + H]⁺ calcd for | J = 1.9 Hz, 1H), 8.08 (dd, J = 8.7, 2.0 Hz, 1H), 7.85-7.64 (m, |
| | | C₂₇H₁₇ClF₆N₆O₃S, | 3H), 7.54-7.31 (m, 3H), 7.13 (s, 1H), 4.09-3.87 (m, 2H), |
| | | 655.0748; found, | 2.51 (s, 3H), 1.24 (s, 1H); ¹⁹F NMR (376 MHz, CDCl₃) |
| | | 655.0754 | δ −58.03, −61.09 |
| J40 | | HRMS-ESI (m/z) | ¹H NMR (400 MHz, CDCl₃) δ 8.53 (d, J = 7.6 Hz, 2H), 8.18 (d, |
| | | [M + H]⁺ calcd for | J = 1.9 Hz, 1H), 8.08 (dd, J = 8.7, 2.0 Hz, 1H), 7.86-7.65 (m, |
| | | C₂₈H₁₉ClF₆N₆O₄S, | 4H), 7.51-7.33 (m, 3H), 7.13 (s, 1H), 4.07-3.88 (m, 2H), |
| | | 685.0854; found, | 2.51 (s, 3H), 1.24 (s, 2H); ¹⁹F NMR (376 MHz, CDCl₃) |
| | | 685.0859 | δ −58.03, −74.11 |
| J41 | | HRMS-ESI (m/z) | ¹H NMR (400 MHz, CDCl₃) δ 8.54 (s, 1H), 8.52-8.42 (m, 1H), |
| | | [M + H]⁺ calcd for | 8.04-7.84 (m, 2H), 7.84-7.73 (m, 2H), 7.51 (d, J = 3.0 Hz, |
| | | C₂₉H₂₄F₄N₆O₃S, | 1H), 7.39 (d, J = 8.6 Hz, 2H), 7.34-7.28 (m, 2H), 6.93 (s, 1H), |
| | | 613.1639; found, | 3.98 (d, J = 2.5 Hz, 2H), 2.45-2.27 (m, 5H), 1.60 (dt, J = 15.3, |
| | | 613.1646 | 7.5 Hz, 2H), 0.92 (t, J = 7.3 Hz, 3H); ¹⁹F NMR (376 MHz, |
| | | | CDCl₃) δ −58.03, −131.08, −135.37 |
| J42 | | | ¹H NMR (400 MHz, CDCl₃) δ 8.54 (s, 1H), 8.47 (t, J = 8.3 Hz, |
| | | | 1H), 8.04-7.86 (m, 2H), 7.86-7.71 (m, 3H), 7.66 (ddd, J = |
| | | | 8.5, 2.1, 0.9 Hz, 1H), 7.49 (d, J = 3.1 Hz, 1H), 7.44-7.32 (m, |
| | | | 3H), 4.12-3.84 (m, 2H); ¹⁹F NMR (376 MHz, CDCl₃) |
| | | | δ −58.03, −61.34, −131.41 |
| J43 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 8.03-7.85 (m, 1H), 7.84-7.72 (m, 1H), 7.52 (d, J = 3.4 |
| | | C₂₉H₂₄F₄N₆O₄S, | Hz, 1H), 7.43-7.36 (m, 1H), 7.36-7.28 (m, 2H), 7.01 (ddd, J = |
| | | 629.1589; found, | 12.8, 8.7, 2.7 Hz, 1H), 6.62 (dd, J = 25.7, 2.6 Hz, 1H), 4.17- |
| | | 629.1596 | 4.04 (m, 1H), 3.99 (d, J = 2.2 Hz, 1H), 3.82 (d, J = 11.5 Hz, |
| | | | 3H), 2.73 (s, 1H), 2.43-2.21 (m, 2H), 1.57 (dq, J = 14.8, 7.3 |
| | | | Hz, 2H), 1.24 (s, 1H), 0.91 (td, J = 7.3, 2.5 Hz, 3H); ¹⁹F NMR |
| | | | (376 MHz, CDCl₃) δ −58.03, −131.05 |
| J44 | | | ¹H NMR (400 MHz, CDCl₃) δ 8.54 (s, 1H), 8.52-8.42 (m, 1H), |
| | | | 7.97 (d, J = 8.6 Hz, 1H), 7.89 (dd, J = 12.1, 1.9 Hz, 1H), 7.83- |
| | | | 7.72 (m, 2H), 7.51 (s, 1H), 7.46-7.28 (m, 3H), 6.93 (s, 2H), |
| | | | 3.98 (d, J = 1.1 Hz, 3H), 2.39 (d, J = 6.5 Hz, 4H), 1.54 (d, J = |
| | | | 15.5 Hz, 3H), 1.33 (dt, J = 14.7, 7.3 Hz, 2H), 1.24 (s, 2H), 0.88 |
| | | | (t, J = 7.3 Hz, 3H); ¹⁹F NMR (376 MHz, CDCl₃) |
| | | | δ −58.03, −131.09 |
| J45 | | | ¹H NMR (400 MHz, CDCl₃) δ 8.54 (s, 1H), 8.47 (d, J = 8.3 Hz, |
| | | | 1H), 8.05-7.92 (m, 1H), 7.89 (dd, J = 12.0, 1.9 Hz, 1H), 7.81- |
| | | | 7.71 (m, 2H), 7.57-7.46 (m, 1H), 7.45-7.35 (m, 2H), 7.31 |
| | | | (t, J = 8.0 Hz, 1H), 7.02 (dd, J = 8.6, 2.7 Hz, 1H), 6.65 (d, J = |
| | | | 2.7 Hz, 1H), 3.98 (d, J = 0.9 Hz, 2H), 3.81 (d, J = 11.1 Hz, 3H), |
| | | | 2.44-2.31 (m, 2H), 1.52 (d, J = 17.2 Hz, 2H), 1.33 (dt, J = |
| | | | 14.8, 7.4 Hz, 1H), 1.24 (s, 1H), 0.89 (td, J = 7.3, 4.1 Hz, 3H); |
| | | | ¹⁹F NMR (376 MHz, CDCl₃) δ −58.03, −131.06 |
| J46 | | HRMS-ESI (m/z) | ¹H NMR (300 MHz, CDCl₃) δ 8.54 (s, 1H), 8.52-8.42 (m, 1H), |
| | | [M + H]⁺ calcd for | 7.97 (d, J = 8.9 Hz, 1H), 7.90 (dd, J = 12.0, 1.8 Hz, 1H), 7.82- |
| | | C₂₈H₂₁ClF₄N₆O₃S, | 7.73 (m, 3H), 7.51 (d, J = 3.6 Hz, 1H), 7.43-7.34 (m, 3H), |
| | | 633.1093; found, | 7.12 (d, J = 2.1 Hz, 1H), 4.00 (d, J = 1.1 Hz, 2H), 2.67 (p, J = |
| | | 633.1097 | 6.9 Hz, 1H), 1.20 (dd, J = 6.9, 3.0 Hz, 6H); ¹⁹F NMR (376 MHz, |
| | | | CDCl₃) δ −58.03, −131.11, −135.37 |
| J47 | | HRMS-ESI (m/z) | ¹H NMR (400 MHz, CDCl₃) δ 8.54 (s, 1H), 8.49 (t, J = 8.3 Hz, |
| | | [M + H]⁺ calcd for | 1H), 7.97 (dd, J = 8.6, 1.5 Hz, 1H), 7.88 (dd, J = 12.0, 1.9 Hz, |
| | | C₃₀H₂₆F₄N₆O₃S, | 1H), 7.82-7.75 (m, 2H), 7.50 (d, J = 3.0 Hz, 1H), 7.43-7.34 |
| | | 627.1796; found, | (m, 2H), 6.93 (s, 1H), 3.98 (d, J = 1.7 Hz, 2H), 2.40 (s, 3H), |
| | | 627.1796 | 2.32-2.24 (m, 2H), 1.82 (dq, J = 13.7, 6.8 Hz, 1H), 1.25 (d, J = |
| | | | 7.0 Hz, 2H), 0.88 (dd, J = 6.6, 2.7 Hz, 6H); ¹⁹F NMR (376 |
| | | | MHz, CDCl₃) δ −57.93, −58.03, −58.04-−58.12 (m), −131.18 |
| J48 | | | ¹H NMR (400 MHz, CDCl₃) δ 8.54 (s, 1H), 8.47 (td, J = 8.4, 3.0 |
| | | | Hz, 1H), 7.97 (dd, J = 8.7, 1.6 Hz, 1H), 7.88 (dt, J = 12.0, 1.5 |
| | | | Hz, 1H), 7.82-7.75 (m, 2H), 7.48 (dd, J = 21.4, 3.0 Hz, 1H), |
| | | | 7.42-7.35 (m, 2H), 7.33 (d, J = 8.8 Hz, 1H), 7.07 (ddd, J = |
| | | | 8.8, 2.9, 1.1 Hz, 1H), 6.62 (dd, J = 2.7, 1.7 Hz, 1H), 4.02-3.96 |
| | | | (m, 2H), 3.81 (d, J = 16.2 Hz, 3H), 2.34 (dtd, J = 10.4, 6.6, 3.5 |
| | | | Hz, 1H), 1.32-1.21 (m, 2H), 1.16 (dd, J = 12.4, 6.9 Hz, 3H), |
| | | | 0.77 (dt, J = 10.5, 7.4 Hz, 3H); ¹⁹F NMR (376 MHz, CDCl₃) |
| | | | δ −58.03, −131.09, −131.36 |
| J49 | | HRMS-ESI (m/z) | ¹H NMR (400 MHz, CDCl₃) δ 8.54 (s, 1H), 8.47 (td, J = 8.3, 4.8 |
| | | [M + H]⁺ calcd for | Hz, 1H), 7.97 (dd, J = 8.6, 1.5 Hz, 1H), 7.88 (ddd, J = 12.0, 3.5, |
| | | C₂₉H₂₄F₄N₆O₄S, | 1.8 Hz, 1H), 7.82-7.71 (m, 2H), 7.57-7.46 (m, 2H), 7.40 (td, |
| | | 629.1589; found, | J = 10.8, 9.3, 2.0 Hz, 4H), 6.93 (s, 1H), 4.21-4.05 (m, 2H), |
| | | 629.1593 | 4.02-3.94 (m, 2H), 3.23-3.03 (m, 4H), 2.43 (s, 3H), 1.45- |
| | | | 1.30 (m, 4H); ¹⁹F NMR (376 MHz, CDCl₃) |
| | | | δ −58.03, −131.12, −131.56 |
| J50 | | HRMS-ESI (m/z) | ¹H NMR (400 MHz, CDCl₃) δ 8.54 (s, 1H), 8.47 (t, J = 8.4 Hz, |
| | | [M + H]⁺ calcd for | 1H), 8.03 (d, J = 8.3 Hz, 1H), 8.01-7.93 (m, 2H), 7.89 (dd, J = |
| | | C₂₇H₁₄F₁₀N₆O₃S, | 12.0, 1.8 Hz, 1H), 7.82-7.74 (m, 2H), 7.62 (s, 1H), 7.46 (d, J = |
| | | 693.0761; found, | 3.1 Hz, 1H), 7.42-7.34 (m, 2H), 4.09-3.93 (m, 2H); ¹⁹F |
| | | 693.0768 | NMR (376 MHz, CDCl₃) δ −58.03, −61.82, −63.08, −131.47 |
| J51 | | HRMS-ESI (m/z) | ¹H NMR (400 MHz, CDCl₃) δ 8.54 (s, 1H), 8.49 (td, J = 8.4, 2.5 |
| | | [M + H]⁺ calcd for | Hz, 1H), 7.96 (d, J = 8.8 Hz, 1H), 7.88 (dd, J = 12.0, 1.8 Hz, |
| | | C₃₀H₂₆F₄N₆O₄S, | 1H), 7.82-7.71 (m, 2H), 7.52 (s, 1H), 7.38 (d, J = 8.6 Hz, 2H), |
| | | 643.1745; found, | 7.00 (s, 1H), 6.95 (d, J = 8.5 Hz, 1H), 4.33 (qd, J = 6.0, 2.5 Hz, |
| | | 643.1748 | 1H), 3.94 (d, J = 1.5 Hz, 2H), 2.36 (s, 3H), 1.73-1.57 (m, 2H), |
| | | | 1.23 (dd, J = 6.1, 4.9 Hz, 3H), 0.89 (q, J = 7.5 Hz, 3H); ¹⁹F |
| | | | NMR (376 MHz, CDCl₃) δ −58.03, 131.33 (d, J = 10.7 Hz), −131.34 |
| J52 | | HRMS-ESI (m/z) | ¹H NMR (400 MHz, CDCl₃) δ 8.55 (s, 1H), 8.49 (t, J = 8.3 Hz, |
| | | [M + H]⁺ calcd for | 1H), 7.97 (dd, J = 8.5, 1.6 Hz, 1H), 7.89 (dd, J = 12.0, 1.8 Hz, |
| | | C₃₀H₂₄F₆N₆O₃S, | 1H), 7.83-7.74 (m, 2H), 7.53 (d, J = 2.9 Hz, 1H), 7.39 (d, J = |
| | | 663.1608; found, | 8.9 Hz, 2H), 7.35-7.28 (m, 2H), 6.93 (s, 1H), 3.98 (d, J = 2.5 |
| | | 663.1604 | Hz, 2H), 2.39 (d, J = 6.6 Hz, 4H), 2.01 (s, 2H), 1.61 (dd, J = |
| | | | 15.1, 7.6 Hz, 2H), 0.92 (t, J = 7.3 Hz, 3H); ¹⁹F NMR (376 MHz, |
| | | | CDCl₃) δ −85.90, −87.85, −131.06 |
| J53 | | HRMS-ESI (m/z) | ¹H NMR (400 MHz, CDCl₃) δ 8.52 (d, J = 11.8 Hz, 1H), 8.19 (d, |
| | | [M + H]⁺ calcd for | J = 8.5 Hz, 1H), 8.09-7.95 (m, 2H), 7.85-7.72 (m, 2H), 7.38 |
| | | C₃₀H₂₇F₃N₆O₃S, | (d, J = 8.5 Hz, 2H), 7.30 (d, J = 7.9 Hz, 1H), 7.08 (s, 1H), 7.01- |
| | | 609.189; found, | 6.70 (m, 2H), 4.08-3.79 (m, 3H), 2.38 (d, J = 13.3 Hz, 4H), |
| | | 609.1868 | 2.25 (d, J = 5.9 Hz, 3H), 1.61 (p, J = 7.5 Hz, 2H), 0.93 (td, J = |
| | | | 7.5, 3.8 Hz, 3H); ¹⁹F NMR (376 MHz, CDCl₃) δ −58.03 |
| J54 | | HRMS-ESI (m/z) | ¹H NMR (400 MHz, CDCl₃) δ 8.53 (s, 1H), 8.19 (d, J = 8.5 Hz, |
| | | [M + H]⁺ calcd for | 1H), 8.09-7.92 (m, 2H), 7.79 (d, J = 8.7 Hz, 2H), 7.38 (dd, J = |
| | | C₃₀H₂₇F₃N₆O₄S, | 8.8, 4.2 Hz, 3H), 7.16-6.98 (m, 2H), 6.63 (d, J = 2.7 Hz, 1H), |
| | | 625.1839; found, | 3.98 (s, 2H), 3.82 (s, 3H), 2.65 (p, J = 6.9 Hz, 1H), 2.26 (s, 3H), |
| | | 625.181 | 1.19 (dd, J = 12.2, 6.8 Hz, 6H); ¹⁹F NMR (376 MHz, CDCl₃) |
| | | | δ −58.03 |
| J55 | | HRMS-ESI (m/z) | ¹H NMR (400 MHz, CDCl₃) δ 8.53 (d, J = 2.4 Hz, 1H), 8.19 (d, |
| | | [M + H]⁺ calcd for | J = 8.7 Hz, 1H), 8.10-7.93 (m, 2H), 7.79 (dd, J = 9.1, 2.4 Hz, |
| | | C₂₉H₂₂F₆N₆O₄S | 2H), 7.45-7.28 (m, 3H), 7.07 (d, J = 10.4 Hz, 2H), 6.99 (d, J = |
| | | 665.14; found, | 8.4 Hz, 1H), 4.36 (dt, J = 19.4, 9.7 Hz, 2H), 4.07-3.86 (m, |
| | | 665.1425 | 2H), 2.39 (d, J = 2.4 Hz, 3H), 2.27 (s, 3H); ¹⁹F NMR (376 MHz, |
| | | | CDCl₃) δ −58.03, −74.02 |
| J56 | | HRMS-ESI (m/z) | ¹H NMR (400 MHz, CDCl₃) δ 8.53 (s, 1H), 8.20 (d, J = 8.5 Hz, |
| | | [M + H]⁺ calcd for | 1H), 8.09-7.93 (m, 2H), 7.85-7.73 (m, 2H), 7.38 (d, J = 8.6 |
| | | C₃₀H₂₄F₆N₆O₄S | Hz, 2H), 7.28 (s, 1H), 7.13-7.00 (m, 2H), 6.96 (d, J = 8.5 Hz, |
| | | 679.1557; found, | 1H), 4.22 (t, J = 6.3 Hz, 2H), 3.91 (d, J = 1.8 Hz, 2H), 2.55 (qt, |
| | | 679.1572 | J = 10.6, 6.1 Hz, 2H), 2.38 (s, 3H), 2.27 (s, 3H); ¹⁹F NMR (376 |
| | | | MHz, CDCl₃) δ −58.03, −64.59 |
| J57 | | HRMS-ESI (m/z) | ¹H NMR (400 MHz, CDCl₃) δ 8.50-8.42 (m, 2H), 8.03-7.82 |
| | | [M + H]⁺ calcd for | (m, 2H), 7.75-7.60 (m, 2H), 7.51 (d, J = 2.9 Hz, 1H), 7.35 (dt, |
| | | C₃₀H₂₆F₄N₆O₃S, | J = 25.0, 8.1 Hz, 2H), 7.15-7.00 (m, 2H), 6.97-6.82 (m, 1H), |
| | | 627.1796; found, | 4.41 (q, J = 8.0 Hz, 2H), 3.99 (d, J = 2.3 Hz, 2H), 2.66 (p, J = |
| | | 627.1835 | 6.9 Hz, 1H), 2.37 (d, J = 13.4 Hz, 3H), 1.27-1.11 (m, 6H); ¹⁹F |
| | | | NMR (376 MHz, CDCl₃) δ −73.84, −131.14 |
| J58 | | HRMS-ESI (m/z) | ¹H NMR (400 MHz, CDCl₃) δ 8.55-8.37 (m, 2H), 8.03-7.80 |
| | | [M + H]⁺ calcd for | (m, 2H), 7.75-7.61 (m, 2H), 7.52 (d, J = 3.0 Hz, 1H), 7.39 (d, |
| | | C₃₀H₂₆F₄N₆O₄S, | J = 8.8 Hz, 1H), 7.08 (dq, J = 7.6, 3.1, 2.7 Hz, 3H), 6.61 (d, J = |
| | | 643.1745; found, | 2.7 Hz, 1H), 4.42 (q, J = 8.0 Hz, 2H), 3.99 (d, J = 2.0 Hz, 2H), |
| | | 643.1764 | 3.83 (s, 3H), 2.63 (p, J = 6.8 Hz, 1H), 1.18 (t, J = 6.4 Hz, 6H); |
| | | | ¹⁹F NMR (376 MHz, CDCl₃) δ −73.84, −131.14 |
| J59 | | HRMS-ESI (m/z) | ¹H NMR (400 MHz, CDCl₃) δ 8.54 (s, 1H), 8.50 (t, J = 8.4 Hz, |
| | | [M + H]⁺ calcd for | 1H), 7.96 (dd, J = 8.5, 1.5 Hz, 1H), 7.88 (dd, J = 12.0, 1.9 Hz, |
| | | C₂₈H₂₂F₄N₆O₄S, | 1H), 7.83-7.74 (m, 2H), 7.52 (d, J = 3.0 Hz, 1H), 7.38 (d, J = |
| | | 615.1432; found, | 8.6 Hz, 2H), 7.25-7.21 (m, 1H), 7.03-6.92 (m, 2H), 4.06 (q, |
| | | 615.1468 | J = 7.0 Hz, 2H), 4.01-3.86 (m, 2H), 2.37 (s, 3H), 1.31 (t, J = |
| | | | 7.0 Hz, 3H); ¹⁹F NMR (376 MHz, CDCl₃) δ −58.03, −131.31 |
| J60 | | HRMS-ESI (m/z) | ¹H NMR (400 MHz, CDCl₃) δ 8.53 (d, J = 0.6 Hz, 1H), 8.17 (dd, |
| | | [M + H]⁺ calcd for | J = 8.5, 3.2 Hz, 1H), 8.08-7.94 (m, 2H), 7.85-7.73 (m, 2H), |
| | | C₃₀H₂₇F₃N₆O₄S, | 7.54 (dd, J = 8.0, 3.1 Hz, 1H), 7.38 (d, J = 8.5 Hz, 3H), 7.10- |
| | | 625.1839; found, | 6.89 (m, 2H), 4.23-4.06 (m, 2H), 4.04-3.92 (m, 2H), 3.21 (s, |
| | | 625.1871 | 1H), 3.12 (s, 1H), 2.42 (d, J = 2.0 Hz, 3H), 2.22 (d, J = 23.7 Hz, |
| | | | 3H), 1.38 (dd, J = 26.1, 6.4 Hz, 3H); ¹⁹F NMR (376 MHz, |
| | | | CDCl₃) δ −58.03 |
| J61 | | HRMS-ESI (m/z) | ¹H NMR (400 MHz, CDCl₃) δ 8.62 (d, J = 10.2 Hz, 1H), 8.49 (t, |
| | | [M + H]⁺ calcd for | J = 8.3 Hz, 1H), 7.98 (d, J = 8.6 Hz, 1H), 7.94-7.84 (m, 2H), |
| | | C₂₉H₂₄F₄N₆O₂S, | 7.85-7.74 (m, 2H), 7.53 (s, 1H), 7.35 (dt, J = 25.4, 8.8 Hz, |
| | | 597.169; found, | 2H), 6.95-6.79 (m, 1H), 4.20-4.05 (m, 1H), 3.99 (d, J = 2.3 |
| | | 597.1695 | Hz, 2H), 2.66 (p, J = 6.9 Hz, 1H), 2.37 (d, J = 14.1 Hz, 3H), |
| | | | 1.27-1.11 (m, 6H); ¹⁹F NMR (376 MHz, CDCl₃) δ −62.52, −130.98 |
| J62 | | HRMS-ESI (m/z) | ¹H NMR (400 MHz, CDCl₃) δ 8.63 (s, 1H), 8.48 (td, J = 8.4, 4.8 |
| | | [M + H]⁺ calcd for | Hz, 1H), 7.98 (d, J = 8.6 Hz, 1H), 7.90 (d, J = 8.7 Hz, 2H), 7.80 |
| | | C₂₉H₂₄F₄N₆O₃S, | (d, J = 8.5 Hz, 2H), 7.60-7.47 (m, 1H), 7.40 (dd, J = 14.4, 9.3 |
| | | 613.1639; found, | Hz, 1H), 7.27 (m, 1H), 6.93 (s, 1H), 4.23-4.08 (m, 2H), 4.06- |
| | | 613.1659 | 3.92 (m, 2H), 3.15 (d, J = 22.3 Hz, 3H), 2.43 (s, 3H), 1.37 (dd, |
| | | | J = 16.3, 6.4 Hz, 3H); ¹⁹F NMR (376 MHz, CDCl₃) δ −62.52, −131.06 |
| J63 | | HRMS-ESI (m/z) | ¹H NMR (400 MHz, CDCl₃) δ 8.63 (s, 1H), 8.50 (t, J = 8.4 Hz, |
| | | [M + H]⁺ calcd for | 1H), 7.98 (d, J = 8.6 Hz, 1H), 7.94-7.86 (m, 2H), 7.80 (d, J = |
| | | C₂₈H₁₉F₇N₆O₃S, | 8.6 Hz, 2H), 7.50 (d, J = 3.1 Hz, 1H), 7.31 (dd, J = 8.6, 2.0 Hz, |
| | | 653.12; found, | 2H), 7.13-7.04 (m, 1H), 6.99 (d, J = 8.5 Hz, 1H), 4.46-4.25 |
| | | 653.1202 | (m, 2H), 4.05-3.87 (m, 2H), 2.40 (s, 3H); ¹⁹F NMR (376 MHz, |
| | | | CDCl₃) δ −62.52, −74.17, −131.52 |
| J64 | | HRMS-ESI (m/z) | ¹H NMR (400 MHz, CDCl₃) δ 8.63 (s, 1H), 8.51 (t, J = 8.4 Hz, |
| | | [M + H]⁺ calcd for | 1H), 7.98 (d, J = 8.7 Hz, 1H), 7.95-7.85 (m, 2H), 7.80 (d, J = |
| | | C₂₉H₂₁F₇N₆O₃S, | 8.5 Hz, 2H), 7.51 (d, J = 3.0 Hz, 1H), 7.34-7.28 (m, 2H), 7.08- |
| | | 667.1357; found, | 6.91 (m, 2H), 4.22 (tt, J = 6.9, 3.3 Hz, 2H), 3.94 (d, J = 1.7 |
| | | 667.1378 | Hz, 2H), 2.66-2.43 (m, 2H), 2.38 (s, 3H); ¹⁹F NMR (376 MHz, |
| | | | CDCl₃) δ −62.52, −64.61 , −131.44 |
| J65 | | HRMS-ESI (m/z) | ¹H NMR (400 MHz, CDCl₃) δ 8.63 (s, 1H), 8.48 (td, J = 8.3, 2.8 |
| | | [M + H]⁺ calcd for | Hz, 1H), 7.98 (d, J = 8.6 Hz, 1H), 7.94-7.84 (m, 3H), 7.80 (d, |
| | | C₃₀H₂₆F₄N₆O₂S, | J = 8.7 Hz, 2H), 7.57-7.40 (m, 1H), 7.32 (t, J = 1.3 Hz, 3H), |
| | | 611.1847; found, | 6.98-6.81 (m, 1H), 4.07-3.89 (m, 2H), 2.40 (s, 4H), 1.73- |
| | | 611.1872 | 1.56 (m, 1H), 1.17 (dd, J = 16.4, 6.9 Hz, 3H), 0.78 (dt, J = 15.0, |
| | | | 7.4 Hz, 3H); ¹⁹F NMR (376 MHz, CDCl₃) δ −62.52, −131.35 |
| J66 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (dd, J = 8.7, 1.6 Hz, 1H), 7.88 (dd, J = 12.0, 1.9 Hz, |
| | | C₂₈H₂₂F₄N₆O₄S, | 1H), 7.82-7.74 (m, 2H), 7.50 (d, J = 3.0 Hz, 1H), 7.44-7.35 |
| | | 615.1432; found, | (m, 3H), 7.34-7.28 (m, 1H), 7.06-6.95 (m, 1H), 4.43-4.24 |
| | | 615.1454 | (m, 2H), 4.03-3.87 (m, 2H), 3.27 (s, 3H), 2.44 (s, 3H); ¹⁹F |
| | | | NMR (376 MHz, CDCl₃) δ −58.03, −131.21 |
| J67 | | HRMS-ESI (m/z) | ¹H NMR (400 MHz, CDCl₃) δ 8.53 (s, 1H), 8.20 (d, J = 8.6 Hz, |
| | | [M + H]⁺ calcd for | 1H), 8.09-7.94 (m, 2H), 7.83-7.72 (m, 2H), 7.40 (dd, J = |
| | | C₂₉H₂₅F₃N₆O₄S, | 17.1, 8.2 Hz, 3H), 7.31 (d, J = 8.0 Hz, 1H), 7.05 (d, J = 20.0 |
| | | 611.1683; found, | Hz, 2H), 4.46-4.21 (m, 2H), 3.94 (d, J = 3.3 Hz, 2H), 3.29 (s, |
| | | 611.1697 | 3H), 2.43 (s, 3H), 2.26 (s, 3H); ¹⁹F NMR (376 MHz, CDCl₃) δ −58.03 |
| J68 | | | ¹H NMR (400 MHz, CDCl₃) δ 8.53 (s, 1H), 8.18 (d, J = 8.5 Hz, |
| | | | 1H), 8.08-7.96 (m, 2H), 7.85-7.74 (m, 2H), 7.44-7.29 (m, |
| | | | 3H), 7.15 (s, 1H), 6.88 (dd, J = 8.8, 2.8 Hz, 1H), 6.38 (d, J = |
| | | | 2.7 Hz, 1H), 3.97 (s, 2H), 2.96 (s, 6H), 2.61 (p, J = 6.9 Hz, 1H), |
| | | | 2.26 (s, 3H), 1.18 (dd, J = 15.4, 6.8 Hz, 6H); ¹⁹F NMR (376 |
| | | | MHz, CDCl₃) δ −58.03 |
| J69 | | HRMS-ESI (m/z) | ¹H NMR (400 MHz, CDCl₃) δ 8.54 (s, 1H), 8.49 (t, J = 8.3 Hz, |
| | | [M + H]⁺ calcd for | 1H), 7.97 (d, J = 8.7 Hz, 1H), 7.88 (dd, J = 12.0, 1.9 Hz, 1H), |
| | | C₃₂H₃₁F₄N₇O₃S, | 7.83-7.75 (m, 2H), 7.60 (d, J = 3.0 Hz, 1H), 7.39 (d, J = 8.5 |
| | | 670.2218; found, | Hz, 2H), 7.29 (d, J = 8.8 Hz, 1H), 6.82 (dd, J = 8.9, 2.8 Hz, 1H), |
| | | 670.2197 | 6.28 (d, J = 2.8 Hz, 1H), 3.99 (d, J = 2.0 Hz, 2H), 3.35 (hept, J = |
| | | | 7.6 Hz, 4H), 2.56 (p, J = 6.8 Hz, 1H), 1.21-1.14 (m, 12H); |
| | | | ¹⁹F NMR (376 MHz, CDCl₃) δ −58.03, −130.74 |
| J70 | | HRMS-ESI (m/z) | ¹H NMR (400 MHz, CDCl₃) δ 8.53 (s, 1H), 8.16 (d, J = 8.5 Hz, |
| | | [M + H]⁺ calcd for | 1H), 8.10-7.95 (m, 2H), 7.86-7.73 (m, 2H), 7.38 (d, J = 8.3 |
| | | C₃₁H₂₉F₃N₆O₃S, | Hz, 3H), 7.35-7.28 (m, 1H), 7.14 (s, 1H), 6.92 (s, 1H), 3.97 (s, |
| | | 623.2047; found, | 2H), 2.69 (p, J = 6.9 Hz, 1H), 2.58 (q, J = 7.5 Hz, 2H), 2.39 (s, |
| | | 623.202 | 3H), 1.32-1.05 (m, 9H); ¹⁹F NMR (376 MHz, CDCl₃) |
| | | | δ −47.79, −57.81, −58.04, −58.50, −114.64, −175.43, −207.71 |
| J71 | | HRMS-ESI (m/z) | ¹H NMR (400 MHz, CDCl₃) δ 8.53 (s, 1H), 8.17 (d, J = 8.5 Hz, |
| | | [M + H]⁺ calcd for | 1H), 8.08-7.95 (m, 2H), 7.84-7.75 (m, 2H), 7.38 (dd, J = 8.7, |
| | | C₃₁H₂₉F₃N₆O₄S, | 3.5 Hz, 3H), 7.15 (s, 1H), 7.07 (dd, J = 8.8, 2.7 Hz, 1H), 6.63 |
| | | 639.1996; found, | (d, J = 2.7 Hz, 1H), 3.97 (s, 2H), 3.82 (s, 3H), 2.63 (dq, J = |
| | | 639.1977 | 32.1, 7.6, 7.1 Hz, 3H), 1.32-1.03 (m, 8H); ¹⁹F NMR (376 MHz, |
| | | | CDCl₃) δ −58.04, −138.72, −173.65 |
| J72 | | HRMS-ESI (m/z) | ¹H NMR (400 MHz, CDCl₃) δ 8.54 (s, 1H), 8.17 (d, J = 8.5 Hz, |
| | | [M + H]⁺ calcd for | 1H), 8.09-7.92 (m, 2H), 7.88-7.72 (m, 2H), 7.48-7.29 (m, |
| | | C₃₀H₂₇F₃N₆O₄S, | 4H), 7.11 (s, 1H), 7.03 (s, 1H), 4.48-4.22 (m, 3H), 3.94 (d, J = |
| | | 625.1839; found, | 3.1 Hz, 2H), 3.29 (s, 3H), 2.59 (q, J = 7.5 Hz, 2H), 2.43 (s, 3H), |
| | | 625.1819 | 1.25 (t, J = 7.5 Hz, 3H); ¹⁹F NMR (376 MHz, CDCl₃) δ −58.03, −58.03 |
| J73 | | HRMS-ESI (m/z) | ¹H NMR (400 MHz, CDCl₃) δ 8.54 (s, 1H), 8.17 (d, J = 8.5 Hz, |
| | | [M + H]⁺ calcd for | 1H), 8.09-7.96 (m, 2H), 7.84-7.76 (m, 2H), 7.38 (d, J = 8.3 |
| | | C₃₁H₂₆F₆N₆O₄S, | Hz, 2H), 7.29 (s, 1H), 7.11 (s, 1H), 7.05 (s, 1H), 6.97 (d, J = 8.4 |
| | | 693.1713; found, | Hz, 1H), 4.22 (t, J = 6.3 Hz, 2H), 3.91 (d, J = 1.9 Hz, 2H), 2.67- |
| | | 693.1701 | 2.47 (m, 4H), 2.38 (s, 3H), 1.26 (t, J = 7.5 Hz, 3H); ¹⁹F NMR |
| | | | (376 MHz, CDCl₃) δ −58.01, −58.03, −64.60 |
| J74 | | HRMS-ESI (m/z) | ¹H NMR (400 MHz, CDCl₃) δ 8.54 (d, J = 0.7 Hz, 1H), 8.16 (dd, |
| | | [M + H]⁺ calcd for | J = 8.5, 3.3 Hz, 1H), 8.02 (dd, J = 18.3, 6.9 Hz, 2H), 7.86- |
| | | C₃₁H₂₉F₃N₆O₄S, | 7.74 (m, 2H), 7.54 (dd, J = 8.0, 3.4 Hz, 1H), 7.38 (d, J = 8.5 Hz, |
| | | 639.1996; found, | 3H), 7.08 (d, J = 19.3 Hz, 1H), 6.94 (d, J = 4.1 Hz, 1H), 4.29- |
| | | 639.197 | 4.08 (m, 2H), 4.05-3.89 (m, 2H), 3.16 (d, J = 35.6 Hz, 3H), |
| | | | 2.55 (dq, J = 22.3, 7.6 Hz, 2H), 2.43 (s, 3H), 1.38 (dd, J = 25.4, |
| | | | 6.4 Hz, 3H), 1.30-1.11 (m, 3H); ¹⁹F NMR (376 MHz, CDCl₃) |
| | | | δ −10.58, −31.77, −58.03, −179.94, −193.69, −209.87 |
| J75 | | HRMS-ESI (m/z) | ¹H NMR (400 MHz, CDCl₃) δ 8.54 (s, 1H), 8.16 (d, J = 8.5 Hz, |
| | | [M + H]⁺ calcd for | 1H), 8.10-7.96 (m, 2H), 7.85-7.74 (m, 2H), 7.38 (d, J = 8.5 |
| | | C₃₀H₂₄F₆N₆O₄S, | Hz, 2H), 7.30 (d, J = 8.4 Hz, 1H), 7.14-7.05 (m, 2H), 7.00 (d, |
| | | 679.1557; found, | J = 8.5 Hz, 1H), 4.50-4.24 (m, 2H), 4.02-3.85 (m, 2H), 2.60 |
| | | 679.1543 | (q, J = 7.5 Hz, 2H), 2.40 (s, 3H), 1.25 (t, J = 7.6 Hz, 3H); ¹⁹F |
| | | | NMR (376 MHz, CDCl₃) δ −58.03, −74.04 |
| J76 | 232-235 | HRMS-ESI (m/z) | ¹H NMR (400 MHz, CDCl₃) δ 8.55 (s, 1H), 8.46 (t, J = 8.3 Hz, |
| | | [M + H]⁺ calcd for | 1H), 7.98 (d, J = 8.7 Hz, 1H), 7.90 (d, J = 11.6 Hz, 1H), 7.83- |
| | | C₂₉H₂₂F₄N₇O₃S, | 7.74 (m, 3H), 7.62 (d, J = 8.2 Hz, 1H), 7.48 (s, 1H), 7.44 (d, J = |
| | | 624.1435; found, | 1.8 Hz, 1H), 7.42-7.36 (m, 2H), 4.03 (s, 2H), 2.76 (dt, J = |
| | | 624.1440 | 13.8, 7.0 Hz, 1H), 1.32-1.15 (m, 6H); ¹⁹F NMR (376 MHz, |
| | | | CDCl₃) δ −58.02, −131.27 |
| J77 | | HRMS-ESI (m/z) | ¹H NMR (400 MHz, CDCl₃) δ 8.54 (s, 1H), 8.47 (t, J = 8.4 Hz, |
| | | [M + H]⁺ calcd for | 1H), 8.10 (dd, J = 8.3, 1.9 Hz, 1H), 7.97 (d, J = 8.5 Hz, 1H), |
| | | C₃₀H₂₄F₄N₆O₄S, | 7.88 (dd, J = 12.0, 1.8 Hz, 1H), 7.82-7.74 (m, 2H), 7.71 (d, J = |
| | | 641.1589; found, | 1.8 Hz, 1H), 7.61 (d, J = 8.3 Hz, 1H), 7.46 (d, J = 3.1 Hz, 1H), |
| | | 641.1589 | 7.38 (d, J = 8.5 Hz, 2H), 4.03 (s, 2H), 2.76 (p, J = 6.8 Hz, 1H), |
| | | | 2.62 (s, 3H), 1.24 (t, J = 6.7 Hz, 6H; ¹⁹F NMR (376 MHz, |
| | | | CDCl₃) δ −58.02, −131.30 |
| J78 | | HRMS-ESI (m/z) | ¹H NMR (400 MHz, CDCl₃) δ 8.56 (s, 1H), 7.78 (d, J = 8.7 Hz, |
| | | [M + H]⁺ calcd for | 4H), 7.40 (d, J = 8.6 Hz, 2H), 7.32 (s, 1H), 6.89 (d, J = 30.1 Hz, |
| | | C₂₉H₂₃F₅N₆O₃S, | 2H), 3.97 (d, J = 2.2 Hz, 2H), 2.68 (s, 1H), 2.39 (s, 3H), 1.29- |
| | | 631.1545; found, | 1.16 (m, 6H); ¹⁹F NMR (376 MHz, CDCl₃) δ −58.02, −116.90 |
| | | 631.1549 | |
| J79 | | HRMS-ESI (m/z) | ¹H NMR (400 MHz, CDCl₃) δ 8.56 (s, 1H), 7.79 (d, J = 8.5 Hz, |
| | | [M + H]⁺ calcd for | 4H), 7.40 (d, J = 8.5 Hz, 2H), 7.09 (s, 1H), 6.99 (d, J = 7.4 Hz, |
| | | C₂₈H₁₈F₈N₆O₄S, | 1H), 6.80 (s, 1H), 4.37 (s, 2H), 4.03-3.86 (m, 2H), 2.40 (s, |
| | | 687.1055; found, | 3H); ¹⁹F NMR (376 MHz, CDCl₃) δ −58.02, −74.12, −116.96 |
| | | 687.106 | |
| J80 | | HRMS-ESI (m/z) | ¹H NMR (400 MHz, CDCl₃) δ 8.56 (s, 1H), 7.79 (d, J = 8.9 Hz, |
| | | [M + H]⁺ calcd for | 4H), 7.40 (d, J = 8.6 Hz, 2H), 7.04 (s, 1H), 6.95 (s, 1H), 6.82 (s, |
| | | C₃₀H₂₂F₈N₆O₄S, | 1H), 4.11-4.03 (m, 2H), 3.93 (s, 2H), 2.37 (s, 3H), 2.25-2.18 |
| | | 715.1368; found, | (m, 2H), 2.08-1.96 (m, 2H); ¹⁹F NMR (376 MHz, CDCl₃) |
| | | 715.1377 | δ −58.02, −66.31, −116.99 |
| J81 | | HRMS-ESI (m/z) | ¹H NMR (400 MHz, CDCl₃) δ 8.56 (s, 1H), 7.78 (d, J = 8.7 Hz, |
| | | [M + H]⁺ calcd for | 4H), 7.53 (d, J = 8.1 Hz, 1H), 7.40 (d, J = 8.7 Hz, 2H), 6.95 (s, |
| | | C₂₉H₂₃F₅N₆O₄S, | 1H), 6.77 (d, J = 30.6 Hz, 1H), 4.17 (s, 1H), 3.98 (dd, J = 2.8, |
| | | 647.1494; found, | 1.4 Hz, 2H), 3.17 (d, J = 38.1 Hz, 3H), 2.42 (s, 3H), 1.44-1.23 |
| | | 647.1502 | (m, 3H); ¹⁹F NMR (376 MHz, CDCl₃) δ −58.02, −116.95, −117.16 |
| J82 | | HRMS-ESI (m/z) | ¹H NMR (400 MHz, CDCl₃) δ 8.55 (s, 1H), 7.78 (d, J = 8.8 Hz, |
| | | [M + H]⁺ calcd for | 4H), 7.40 (d, J = 8.6 Hz, 2H), 7.32 (s, 1H), 6.90 (s, 2H), 6.38 (s, |
| | | C₃₀H₂₆F₅N₇O₃S, | 1H), 3.97 (d, J = 1.8 Hz, 2H), 2.96 (s, 6H), 2.62 (d, J = 11.1 Hz, |
| | | 660.1811; found, | 1H), 1.24-1.13 (m, 6H); ¹⁹F NMR (376 MHz, CDCl₃) |
| | | 660.1813 | δ −58.02, −116.86 |
| J83 | | HRMS-ESI (m/z) | ¹H NMR (400 MHz, CDCl₃) δ 8.54 (s, 1H), 8.17 (d, J = 8.4 Hz, |
| | | [M + H]⁺ calcd for | 1H), 8.09-7.95 (m, 2H), 7.84-7.74 (m, 2H), 7.35 (dd, J = |
| | | C₃₂H₃₂F₃N₇O₃S, | 21.7, 8.7 Hz, 3H), 7.17 (s, 1H), 6.89 (dd, J = 8.8, 2.7 Hz, 2H), |
| | | 652.2312; found, | 6.38 (d, J = 2.7 Hz, 1H), 3.97 (s, 2H), 2.95 (d, J = 9.1 Hz, 6H), |
| | | 652.2324 | 2.70-2.51 (m, 3H), 1.26-1.10 (m, 9H); ¹⁹F NMR (376 MHz, |
| | | | CDCl₃) δ −58.03 |
| J84 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (d, J = 8.5 Hz, 1H), 7.88 (dd, J = 12.0, 1.9 Hz, 1H), |
| | | C₂₇H₂₁F₄N₇O₃S, | 7.84-7.74 (m, 2H), 7.57 (d, J = 3.0 Hz, 1H), 7.44-7.34 (m, |
| | | 600.1435; found, | 3H), 6.82 (dd, J = 8.4, 2.5 Hz, 1H), 6.62-6.46 (m, 2H), 3.96 |
| | | 600.1444 | (s, 2H), 3.01 (s, 6H); ¹⁹F NMR (376 MHz, CDCl₃) δ −58.02, −130.93 |
| J85 | | HRMS-ESI (m/z) | ¹H NMR (400 MHz, CDCl₃) δ 8.57-8.44 (m, 2H), 8.36 (t, J = |
| | | [M + H]⁺ calcd for | 8.4 Hz, 1H), 8.04-7.90 (m, 2H), 7.84-7.69 (m, 3H), 7.64 (d, |
| | | C₂₉H₂₅F₄N₇O₃S, | J = 8.4 Hz, 1H), 7.58-7.47 (m, 2H), 7.35 (dd, J = 23.5, 8.6 Hz, |
| | | 628.1748; found, | 3H), 7.16 (d, J = 2.2 Hz, 1H), 6.79 (d, J = 4.1 Hz, 1H), 4.02 (d, |
| | | 628.1748 | J = 3.4 Hz, 2H), 3.43 (s, 3H), 2.76 (p, J = 6.9 Hz, 1H), 1.26 (t, J = |
| | | | 7.1 Hz, 3H); ¹⁹F NMR (376 MHz, CDCl₃) δ −58.04, −131.15, −133.11 |
| J86 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 8.01-7.94 (m, 1H), 7.88 (dd, J = 12.0, 1.9 Hz, 1H), 7.82- |
| | | C₂₈H₂₂F₄N₆O₃S, | 7.73 (m, 2H), 7.56-7.45 (m, 3H), 7.42-7.32 (m, 3H), 7.13- |
| | | 599.1483; found, | 7.06 (m, 1H), 4.00 (d, J = 2.0 Hz, 2H), 2.71 (p, J = 6.9 Hz, |
| | | 599.1491 | 1H), 1.22 (t, J = 7.1 Hz, 6H); ¹⁹F NMR (376 MHz, CDCl₃) |
| | | | δ −58.02, −58.03, −131.09 |
| J87 | | HRMS-ESI (m/z) | ¹H NMR (400 MHz, CDCl₃) δ 8.54 (s, 1H), 8.51-8.41 (m, 1H), |
| | | [M + H]⁺ calcd for | 7.97 (d, J = 8.5 Hz, 1H), 7.88 (d, J = 9.8 Hz, 1H), 7.78 (d, J = |
| | | C₂₈H₂₂F₄N₆O₄S, | 8.9 Hz, 1H), 7.71-7.51 (m, 3H), 7.50-7.35 (m, 3H), 7.12 (d, |
| | | 615.1432; found, | J = 8.0 Hz, 1H), 4.20 (t, J = 6.9 Hz, 2H), 4.15-4.07 (m, 1H), |
| | | 615.1434 | 4.01 (d, J = 3.1 Hz, 2H), 3.19 (s, 1H), 3.14 (d, J = 2.7 Hz, 3H), |
| | | | 1.46-1.31 (m, 4H); ¹⁹F NMR (376 MHz, CDCl₃) |
| | | | δ −58.02, −58.03, −131.14, −131.53 |
| J88 | | HRMS-ESI (m/z) | ¹H NMR (400 MHz, CDCl₃) δ 8.54 (s, 1H), 8.46 (q, J = 7.9 Hz, |
| | | [M + H]⁺ calcd for | 1H), 7.97 (d, J = 8.6 Hz, 1H), 7.93-7.70 (m, 4H), 7.63-7.52 |
| | | C₂₈H₂₂F₄N₆O₃S2, | (m, 1H), 7.50-7.34 (m, 4H), 7.15-7.05 (m, 1H), 4.09-3.93 |
| | | 631.1204; found, | (m, 2H), 3.76 (dq, J = 13.8, 6.9 Hz, 1H), 1.89 (s, 1H), 1.82 (s, |
| | | 631.1212 | 2H), 1.54 (d, J = 7.0 Hz, 3H), 1.49 (d, J = 7.0 Hz, 1H); ¹⁹F NMR |
| | | | (376 MHz, CDCl₃) δ −57.98-−58.06 (m), −58.03, −131.21, −131.52 |
| J89 | | HRMS-ESI (m/z) | ¹H NMR (400 MHz, CDCl₃) δ 8.53 (s, 1H), 8.48 (d, J = 8.5 Hz, |
| | | [M + H]⁺ calcd for | 1H), 7.88 (s, 1H), 7.85-7.74 (m, 3H), 7.66 (d, J = 1.8 Hz, 1H), |
| | | C₃₁H₃₀F₃N₇O₄S, | 7.43-7.31 (m, 3H), 6.90 (dd, J = 8.8, 2.8 Hz, 1H), 6.39 (d, J = |
| | | 654.2105; found, | 2.8 Hz, 1H), 4.00-3.89 (m, 5H), 2.97 (s, 6H), 2.61 (p, J = 6.8 |
| | | 654.2111 | Hz, 1H), 1.17 (dd, J = 12.9, 6.8 Hz, 6H); ¹⁹F NMR (376 MHz, |
| | | | CDCl₃) δ −58.03 |
| J90 | | HRMS-ESI (m/z) | ¹H NMR (400 MHz, CDCl₃) δ 8.53 (d, J = 0.7 Hz, 1H), 8.50- |
| | | [M + H]⁺ calcd for | 8.40 (m, 1H), 7.90-7.70 (m, 4H), 7.66 (dd, J = 4.4, 1.7 Hz, |
| | | C₃₀H₂₇F₃N₆O₅S, | 1H), 7.55 (dd, J = 8.0, 1.9 Hz, 1H), 7.38 (d, J = 8.5 Hz, 3H), |
| | | 641.1788; found, | 7.01-6.86 (m, 1H), 4.17 (dq, J = 18.6, 6.4 Hz, 1H), 4.02- |
| | | 641.1788 | 3.95 (m, 2H), 3.91 (d, J = 12.6 Hz, 3H), 3.17 (d, J = 36.3 Hz, |
| | | | 3H), 2.43 (d, J = 2.3 Hz, 3H), 1.37 (dd, J = 25.0, 6.5 Hz, 3H); |
| | | | ¹⁹F NMR (376 MHz, CDCl₃) δ −58.03 |
| J91 | | HRMS-ESI (m/z) | ¹H NMR (400 MHz, CDCl₃) δ 8.53 (s, 1H), 8.49 (d, J = 8.5 Hz, |
| | | [M + H]⁺ calcd for | 1H), 7.88-7.74 (m, 4H), 7.66 (d, J = 1.7 Hz, 1H), 7.43-7.34 |
| | | C₃₀H₂₄F₆N₆O₅S, | (m, 2H), 7.32-7.26 (m, 1H), 7.05 (d, J = 2.2 Hz, 1H), 6.97 (d, |
| | | 695.1506; found, | J = 8.5 Hz, 1H), 4.22 (td, J = 6.4, 1.8 Hz, 2H), 3.92 (d, J = 7.0 |
| | | 695.1508 | Hz, 5H), 2.53 (tdq, J = 10.3, 7.4, 3.5 Hz, 2H), 2.38 (s, 3H); ¹⁹F |
| | | | NMR (376 MHz, CDCl₃) δ −58.03, −64.62 |
| J92 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (dd, J = 8.5, 1.5 Hz, 1H), 7.89 (dd, J = 12.0, 1.8 Hz, |
| | | C₂₉H₂₁F₇N₆O₅S, | 1H), 7.83-7.73 (m, 2H), 7.50 (d, J = 3.1 Hz, 1H), 7.45-7.31 |
| | | 699.1255; found, | (m, 2H), 7.08-6.94 (m, 2H), 6.79 (dd, J = 2.4, 1.0 Hz, 1H), |
| | | 699.1257 | 4.19 (qq, J = 6.4, 3.2, 2.5 Hz, 2H), 3.94 (d, J = 1.7 Hz, 2H), |
| | | | 3.83 (s, 3H), 2.63-2.42 (m, 2H); ¹⁹F NMR (376 MHz, CDCl₃) |
| | | | δ −58.02, −64.59, −131.42 |
| J93 | | HRMS-ESI (m/z) | ¹H NMR (400 MHz, CDCl₃) δ 8.54 (s, 1H), 8.13-7.99 (m, 3H), |
| | | [M + H]⁺ calcd for | 7.85-7.71 (m, 2H), 7.38 (d, J = 8.6 Hz, 2H), 7.19-7.03 (m, |
| | | C₃₂H₂₈F₆N₆O₄S, | 2H), 7.03-6.88 (m, 2H), 4.22 (dt, J = 8.9, 6.4 Hz, 1H), 3.96 |
| | | 707.187; found, | (dd, J = 41.5, 1.6 Hz, 3H), 2.98 (p, J = 6.8 Hz, 1H), 2.66-2.46 |
| | | 707.187 | (m, 2H), 2.36 (d, J = 18.0 Hz, 3H), 1.33-1.19 (m, 6H); ¹⁹F |
| | | | NMR (376 MHz, CDCl₃) δ −58.03, −64.62 |
| J94 | | HRMS-ESI (m/z) | ¹H NMR (400 MHz, CDCl₃) δ 8.54 (s, 1H), 8.13-7.98 (m, 3H), |
| | | [M + H]⁺ calcd for | 7.86-7.73 (m, 2H), 7.54 (dd, J = 8.0, 3.2 Hz, 1H), 7.38 (d, J = |
| | | C₃₂H₃₁F₃N₆O₄S, | 8.4 Hz, 3H), 7.14 (d, J = 18.0 Hz, 1H), 7.03-6.87 (m, 1H), |
| | | 653.2152; found, | 4.18 (dq, J = 18.8, 6.4 Hz, 1H), 3.97 (d, J = 2.7 Hz, 2H), 3.16 |
| | | 653.216 | (d, J = 37.1 Hz, 3H), 2.89 (dp, J = 31.4, 6.8 Hz, 1H), 2.43 (d, J = |
| | | | 1.9 Hz, 3H), 1.38 (dd, J = 27.4, 6.4 Hz, 3H), 1.31-1.14 (m, |
| | | | 6H); ¹⁹F NMR (376 MHz, CDCl₃) δ −58.03 |
| J95 | | HRMS-ESI (m/z) | ¹H NMR (400 MHz, CDCl₃) δ 8.54 (s, 1H), 7.91 (s, 2H), 7.82- |
| | | [M + H]⁺ calcd for | 7.77 (m, 2H), 7.38 (d, J = 8.2 Hz, 3H), 7.32 (d, J = 8.0 Hz, 1H), |
| | | C₃₁H₂₉F₃N₆O₃S, | 6.96 (s, 1H), 6.76 (s, 1H), 3.94 (s, 2H), 2.71 (p, J = 6.9 Hz, 1H), |
| | | 623.2047; found, | 2.40 (s, 3H), 2.30 (s, 6H), 1.26 (d, J = 6.9 Hz, 3H), 1.19 (d, J = |
| | | 623.205 | 6.8 Hz, 3H); ¹⁹F NMR (376 MHz, CDCl₃) δ −58.03 |
| J96 | | HRMS-ESI (m/z) | ¹H NMR (400 MHz, CDCl₃) δ 8.54 (s, 1H), 7.91 (s, 2H), 7.82- |
| | | [M + H]⁺ calcd for | 7.77 (m, 2H), 7.41-7.37 (m, 2H), 7.33 (d, J = 8.7 Hz, 1H), |
| | | C₃₂H₃₂F₃N₇O₃S, | 6.92-6.87 (m, 1H), 6.81 (s, 1H), 6.42 (d, J = 2.8 Hz, 1H), 3.94 |
| | | 652.2312; found, | (s, 2H), 2.97 (s, 6H), 2.63 (p, J = 6.8 Hz, 1H), 2.30 (s, 6H), 1.23 |
| | | 652.2322 | (d, J = 6.8 Hz, 3H), 1.17 (d, J = 6.9 Hz, 3H); ¹⁹F NMR (376 |
| | | | MHz, CDCl₃) δ −58.03 |
| J97 | | HRMS-ESI (m/z) | ¹H NMR (400 MHz, CDCl₃) δ 8.54 (s, 1H), 7.91 (s, 2H), 7.79 (d, |
| | | [M + H]⁺ calcd for | J = 8.9 Hz, 2H), 7.56-7.51 (m, 1H), 7.38 (d, J = 8.2 Hz, 3H), |
| | | C₃₁H₂₉F₃N₆O₄S, | 6.98 (s, 1H), 6.70 (d, J = 23.8 Hz, 1H), 4.18 (dq, J = 20.2, 6.4 |
| | | 639.1996; found, | Hz, 1H), 3.94 (d, J = 4.3 Hz, 2H), 3.18 (d, J = 49.5 Hz, 3H), |
| | | 639.2003 | 2.43 (d, J = 1.6 Hz, 3H), 2.29 (s, 3H), 2.27 (s, 3H), 1.40 (dd, J = |
| | | | 37.6, 6.4 Hz, 3H); ¹⁹F NMR (376 MHz, CDCl₃) δ −58.03 |
| J98 | | HRMS-ESI (m/z) | ¹H NMR (400 MHz, CDCl₃) δ 8.54 (s, 1H), 7.91 (s, 2H), 7.82- |
| | | [M + H]⁺ calcd for | 7.77 (m, 2H), 7.39 (dd, J = 8.6, 4.1 Hz, 3H), 7.07 (dd, J = 8.7, |
| | | C₃₁H₂₉F₃N₆O₄S, | 2.7 Hz, 1H), 6.76 (s, 1H), 6.67 (d, J = 2.7 Hz, 1H), 3.95 (s, 2H), |
| | | 639.1996; found, | 3.83 (s, 3H), 2.68 (p, J = 6.8 Hz, 1H), 2.30 (s, 6H), 1.21 (dd, J = |
| | | 639.2004 | 20.2, 6.8 Hz, 6H); ¹⁹F NMR (376 MHz, CDCl₃) δ −58.03 |
| J99 | | HRMS-ESI (m/z) | ¹H NMR (500 MHz, CDCl₃) δ 8.54 (s, 1H), 8.14-7.99 (m, 3H), |
| | | [M + H]⁺ calcd for | 7.87-7.74 (m, 2H), 7.44-7.29 (m, 4H), 7.20 (s, 1H), 7.00- |
| | | C₃₂H₃₁F₃N₆O₃S, | 6.87 (m, 1H), 3.97 (d, J = 1.0 Hz, 2H), 2.95 (hept, J = 6.8 Hz, |
| | | 637.2203; found, | 1H), 2.70 (hept, J = 6.9 Hz, 1H), 2.37 (d, J = 17.8 Hz, 3H), 1.33- |
| | | 637.2211 | 1.07 (m, 12H); ¹⁹F NMR (471 MHz, CDCl₃) δ −58.03 |
| J100 | | HRMS-ESI (m/z) | ¹H NMR (500 MHz, CDCl₃) δ 8.54 (s, 1H), 8.15-7.98 (m, 3H), |
| | | [M + H]⁺ calcd for | 7.87-7.72 (m, 2H), 7.45-7.33 (m, 2H), 7.21 (s, 1H), 7.16- |
| | | C₃₂H₃₁F₃N₆O₄S, | 7.01 (m, 1H), 6.64 (d, J = 2.7 Hz, 2H), 3.97 (d, J = 1.0 Hz, 2H), |
| | | 653.2152; found, | 3.81 (d, J = 13.9 Hz, 3H), 2.95 (hept, J = 6.9 Hz, 1H), 2.66 (p, J = |
| | | 653.2154 | 6.8 Hz, 1H), 1.38-1.09 (m, 12H); ¹⁹F NMR (471 MHz, |
| | | | CDCl₃) δ −58.03 |
| J101 | | HRMS-ESI (m/z) | ¹H NMR (500 MHz, CDCl₃) δ 8.54 (s, 1H), 8.13-7.97 (m, 2H), |
| | | [M + H]⁺ calcd for | 7.90-7.72 (m, 2H), 7.44-7.29 (m, 2H), 7.00-6.81 (m, 2H), |
| | | C₃₃H₃₄F₃N₇O₃S, | 6.39 (d, J = 2.7 Hz, 2H), 3.97 (d, J = 0.8 Hz, 3H), 2.97 (s, 6H), |
| | | 666.2469; found, | 2.61 (h, J = 6.8 Hz, 2H), 1.33-1.07 (m, 12H); |
| | | 666.2471 | ¹⁹F NMR (471 MHz, CDCl₃) δ −58.03 |
| J102 | | HRMS-ESI (m/z) | ¹H NMR (400 MHz, CDCl₃) δ 8.53 (s, 1H), 8.20 (d, J = 8.5 Hz, |
| | | [M + H]⁺ calcd for | 1H), 8.08-7.95 (m, 2H), 7.87-7.70 (m, 2H), 7.38 (d, J = 8.5 |
| | | C₃₁H₂₇F₆N₇O₄S, | Hz, 2H), 7.10 (s, 1H), 7.00 (d, J = 9.0 Hz, 1H), 6.83 (dd, J = |
| | | 708.1822; found, | 9.1, 3.1 Hz, 1H), 6.58 (d, J = 3.0 Hz, 1H), 4.18 (t, J = 6.2 Hz, |
| | | 708.1827 | 2H), 3.92 (d, J = 2.5 Hz, 2H), 2.95 (s, 6H), 2.52 (ddp, J = 16.5, |
| | | | 10.3, 5.9 Hz, 2H), 2.28 (s, 3H); |
| | | | ¹⁹F NMR (376 MHz, CDCl₃) δ −58.03, −64.57 |
| J103 | | HRMS-ESI (m/z) | ¹H NMR (400 MHz, CDCl₃) δ 8.54 (s, 1H), 8.47 (t, J = 8.4 Hz, |
| | | [M + H]⁺ calcd for | 1H), 7.98 (d, J = 8.7 Hz, 1H), 7.90 (dd, J = 12.1, 1.8 Hz, 1H), |
| | | C₂₉H₁₈F₇N₇O₄S, | 7.86-7.73 (m, 3H), 7.57 (d, J = 2.0 Hz, 1H), 7.47 (d, J = 3.1 |
| | | 694.1102; found, | Hz, 1H), 7.43-7.34 (m, 2H), 7.14 (d, J = 8.7 Hz, 1H), 4.32 (tq, |
| | | 694.1102 | J = 8.5, 3.8 Hz, 2H), 3.96 (s, 2H), 2.72-2.51 (m, 2H); ¹⁹F NMR |
| | | | (376 MHz, CDCl₃) δ −58.02, −64.57, −131.45 |
| J104 | | HRMS-ESI (m/z) | ¹H NMR (400 MHz, CDCl₃) δ 8.54 (s, 1H), 8.47 (t, J = 8.3 Hz, |
| | | [M + H]⁺ calcd for | 1H), 8.17 (dd, J = 8.2, 1.8 Hz, 1H), 7.97 (d, J = 8.5 Hz, 1H), |
| | | C₃₀H₂₄F₄N₆O₅S, | 7.88 (dd, J = 12.0, 1.9 Hz, 1H), 7.78 (dd, J = 8.4, 1.8 Hz, 3H), |
| | | 657.1538; found, | 7.58 (d, J = 8.3 Hz, 1H), 7.46 (d, J = 3.1 Hz, 1H), 7.38 (d, J = |
| | | 657.1534 | 8.5 Hz, 2H), 4.02 (d, J = 1.0 Hz, 2H), 3.93 (s, 3H), 2.76 (p, J = |
| | | | 6.8 Hz, 1H), 1.23 (dd, J = 6.8, 5.6 Hz, 6H); |
| | | | ¹⁹F NMR (376 MHz, CDCl₃) δ −58.02, −131.22 |
| J105 | | HRMS-ESI (m/z) | ¹H NMR (400 MHz, CDCl₃) δ 8.54 (s, 1H), 8.50 (t, J = 8.4 Hz, |
| | | [M + H]⁺ calcd for | 1H), 7.97 (d, J = 8.6 Hz, 1H), 7.88 (dd, J = 12.1, 1.8 Hz, 1H), |
| | | C₃₀H₂₄F₇N₇O₄S, | 7.82-7.74 (m, 2H), 7.54 (d, J = 3.0 Hz, 1H), 7.39 (d, J = 8.5 |
| | | 712.1571; found, | Hz, 2H), 7.01 (d, J = 8.9 Hz, 1H), 6.83 (dd, J = 9.1, 3.0 Hz, 1H), |
| | | 712.1577 | 6.56 (d, J = 3.1 Hz, 1H), 4.17 (tq, J = 6.5, 3.7 Hz, 2H), 3.97- |
| | | | 3.89 (m, 2H), 2.95 (s, 6H), 2.59-2.42 (m, 2H); ¹⁹F NMR (376 |
| | | | MHz, CDCl₃) δ −57.96-−58.04 (m), −58.02, −64.60, −64.60 (d, J = |
| | | | 15.6 Hz), −131.27 |
| J106 | | HRMS-ESI (m/z) | ¹H NMR (400 MHz, CDCl₃) δ 8.54 (s, 1H), 8.49 (t, J = 8.3 Hz, |
| | | [M + H]⁺ calcd for | 1H), 7.97 (d, J = 8.6 Hz, 1H), 7.88 (dd, J = 12.0, 1.8 Hz, 1H), |
| | | C₃₀H₂₇F₄N₇O₃S, | 7.83-7.75 (m, 2H), 7.58 (d, J = 3.1 Hz, 1H), 7.39 (d, J = 8.6 |
| | | 642.1905; found, | Hz, 2H), 7.27 (d, J = 8.7 Hz, 1H), 6.76 (dd, J = 8.5, 2.5 Hz, 1H), |
| | | 642.1912 | 6.27 (d, J = 2.5 Hz, 1H), 3.98 (d, J = 2.9 Hz, 2H), 3.63 (s, 1H), |
| | | | 3.15 (q, J = 7.2 Hz, 2H), 2.57 (p, J = 6.8 Hz, 1H), 1.26 (t, J = |
| | | | 7.1 Hz, 3H), 1.16 (t, J = 6.7 Hz, 6H); |
| | | | ¹⁹F NMR (376 MHz, CDCl₃) δ −58.02, −130.79 |
| J107 | | ESIMS m/z | ¹H NMR (400 MHz, CDCl₃) δ 8.54 (s, 1H), 8.49 (t, J = 8.4 Hz, |
| | | 656 ([M + H]⁺) | 1H), 7.97 (dd, J = 8.9, 1.6 Hz, 1H), 7.88 (dd, J = 12.0, 1.8 Hz, |
| | | | 1H), 7.81-7.75 (m, 2H), 7.58 (s, 1H), 7.39 (d, J = 8.6 Hz, 2H), |
| | | | 7.26 (d, J = 8.5 Hz, 1H), 6.76 (dd, J = 8.6, 2.5 Hz, 1H), 6.27 (d, |
| | | | J = 2.6 Hz, 1H), 3.98 (d, J = 3.0 Hz, 2H), 3.71 (s, 1H), 3.07 (t, J = |
| | | | 7.1 Hz, 2H), 2.56 (p, J = 6.8 Hz, 1H), 1.65 (q, J = 7.3 Hz, 2H), |
| | | | 1.16 (t, J = 6.8 Hz, 6H), 1.00 (t, J = 7.4 Hz, 3H); ¹⁹F NMR (376 |
| | | | MHz, CDCl₃) δ −58.02, −130.77 |
| J108 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (d, J = 8.4 Hz, 1H), 7.88 (d, J = 12.6 Hz, 1H), 7.79 (d, |
| | | C₃₄H₃₅F₄N₇O₃S, | J = 9.0 Hz, 2H), 7.61 (s, 1H), 7.39 (d, J = 8.6 Hz, 2H), 7.29 (s, |
| | | 698.2531; found, | 1H), 6.78 (d, J = 6.1 Hz, 1H), 6.23 (d, J = 2.7 Hz, 1H), 3.99 (s, |
| | | 698.254 | 2H), 3.27-3.15 (m, 4H), 2.62-2.52 (m, 1H), 1.62 (q, J = 7.5 |
| | | | Hz, 4H), 1.16 (t, J = 6.2 Hz, 6H), 0.93 (t, J = 7.4 Hz, 6H); ¹⁹F |
| | | | NMR (376 MHz, CDCl₃) δ −58.02, −130.77 |
| J109 | | HRMS-ESI (m/z) | ¹H NMR (400 MHz, CDCl₃) δ 8.53 (s, 1H), 8.49 (d, J = 8.5 Hz, |
| | | [M + H]⁺ calcd for | 1H), 7.90-7.72 (m, 4H), 7.66 (d, J = 1.7 Hz, 1H), 7.45-7.33 |
| | | C₃₁H₂₇F₆N₇O₅S, | (m, 2H), 7.01 (d, J = 9.1 Hz, 1H), 6.84 (dd, J = 9.1, 3.1 Hz, 1H), |
| | | 724.1771; found, | 6.59 (d, J = 3.1 Hz, 1H), 4.17 (tt, J = 5.8, 2.8 Hz, 2H), 3.98- |
| | | 724.1781 | 3.88 (m, 5H), 2.95 (s, 6H), 2.61-2.39 (m, 2H); ¹⁹F NMR (376 |
| | | | MHz, CDCl₃) δ −58.03, −64.61 |
| J110 | | HRMS-ESI (m/z) | ¹H NMR (400 MHz, CDCl₃) δ 8.54 (s, 1H), 8.17 (d, J = 8.5 Hz, |
| | | [M + H]⁺ calcd for | 1H), 8.08-7.95 (m, 2H), 7.86-7.74 (m, 2H), 7.38 (d, J = 8.5 |
| | | C₃₂H₂₉F₆N₇O₄S, | Hz, 2H), 7.14 (s, 1H), 7.01 (d, J = 9.1 Hz, 1H), 6.83 (dd, J = |
| | | 722.1979; found, | 9.1, 3.1 Hz, 1H), 6.59 (d, J = 3.0 Hz, 1H), 4.18 (t, J = 6.3 Hz, |
| | | 722.1984 | 2H), 3.91 (d, J = 2.8 Hz, 2H), 2.94 (s, 6H), 2.69-2.42 (m, 4H), |
| | | | 1.26 (t, J = 7.5 Hz, 3H); ¹⁹F NMR (376 MHz, CDCl₃) δ −58.03, −64.59 |
| J111 | | HRMS-ESI (m/z) | ¹H NMR (400 MHz, CDCl₃) δ 8.54 (s, 1H), 8.14-8.02 (m, 2H), |
| | | [M + H]⁺ calcd for | 7.87-7.72 (m, 2H), 7.38 (d, J = 8.6 Hz, 2H), 7.17 (s, 1H), 6.98 |
| | | C₃₃H₃₁F₆N₇O₄S, | (dd, J = 24.5, 9.1 Hz, 1H), 6.81 (ddd, J = 20.0, 9.1, 3.1 Hz, 1H), |
| | | 736.2135; found, | 6.55 (dd, J = 40.9, 3.0 Hz, 2H), 4.17 (q, J = 6.6 Hz, 2H), 3.91 |
| | | 736.2142 | (d, J = 2.7 Hz, 2H), 2.95 (s, 6H), 2.59-2.44 (m, 3H), 1.34- |
| | | | 1.22 (m, 6H); ¹⁹F NMR (376 MHz, CDCl₃) δ −58.03, −64.60 |
| J112 | | HRMS-ESI (m/z) | ¹H NMR (500 MHz, CDCl₃) δ 8.54 (s, 1H), 8.49 (t, J = 8.4 Hz, |
| | | [M + H]⁺ calcd for | 1H), 8.01-7.83 (m, 2H), 7.83-7.71 (m, 2H), 7.56 (d, J = 3.0 |
| | | C₃₀H₂₇F₄N₇O₃S, | Hz, 1H), 7.46-7.31 (m, 2H), 6.92 (d, J = 8.7 Hz, 1H), 6.75- |
| | | 642.1905; found, | 6.62 (m, 2H), 4.03-3.87 (m, 2H), 3.04 (s, 5H), 2.63 (p, J = 6.8 |
| | | 642.1912 | Hz, 2H), 1.31-1.12(m, 6H); ¹⁹F NMR (471 MHz, CDCl₃) |
| | | | δ −58.02, −130.90 |
| J113 | | HRMS-ESI (m/z) | ¹H NMR (400 MHz, CDCl₃) δ 8.53 (s, 1H), 8.19 (d, J = 8.6 Hz, |
| | | [M + H]⁺ calcd for | 1H), 8.03 (d, J = 9.0 Hz, 1H), 7.98 (s, 1H), 7.83-7.75 (m, 2H), |
| | | C₃₁H₃₀F₃N₇O₃S, | 7.38 (d, J = 8.5 Hz, 2H), 7.15 (s, 1H), 6.94 (d, J = 8.7 Hz, 1H), |
| | | 638.2156; found, | 6.76-6.61 (m, 2H), 3.95 (s, 2H), 3.04 (s, 6H), 2.66 (p, J = 6.8 |
| | | 638.2162 | Hz, 1H), 2.26 (s, 3H), 1.24 (td, J = 13.4, 13.0, 7.0 Hz, 6H); ¹⁹F |
| | | | NMR (376 MHz, CDCl₃) |
| | | | δ −57.80, −58.03, −58.49, −61.40, −120.91, −152.90 |
| J114 | | HRMS-ESI (m/z) | ¹H NMR (300 MHz, CDCl₃) δ 8.56 (s, 1H), 8.49-8.40 (m, 2H), |
| | | [M + H]⁺ calcd for | 8.35 (d, J = 8.5 Hz, 1H), 7.84-7.77 (m, 2H), 7.54 (s, 1H), 7.39 |
| | | C₃₀H₂₄F₆N₆O₃S, | (dd, J = 8.3, 5.8 Hz, 3H), 7.31 (d, J = 9.0 Hz, 1H), 6.90 (d, J = |
| | | 663.1608; found, | 6.1 Hz, 1H), 4.00 (s, 2H), 2.65 (p, J = 6.9 Hz, 1H), 2.38 (s, 3H), |
| | | 663.1614 | 1.19 (dd, J = 14.8, 6.9 Hz, 6H) |
| J115 | | HRMS-ESI (m/z) | ¹H NMR (300 MHz, CDCl₃) δ 8.57 (s, 1H), 8.49 (d, J = 8.8 Hz, |
| | | [M + H]⁺ calcd for | 1H), 8.41 (s, 1H), 8.34 (d, J = 9.9 Hz, 1H), 7.80 (d, J = 9.0 Hz, |
| | | C₂₉H₁₉F₉N₆O₄S, | 2H), 7.52 (s, 1H), 7.40 (d, J = 8.5 Hz, 2H), 7.30 (d, J = 8.4 Hz, |
| | | 719.1118; found, | 1H), 7.07 (s, 1H), 6.99 (d, J = 8.6 Hz, 1H), 4.36 (q, J = 8.2 Hz, |
| | | 719.1132 | 2H), 3.97 (d, J = 3.0 Hz, 2H), 2.40 (s, 3H) |
| J116 | | HRMS-ESI (m/z) | ¹H NMR (300 MHz, CDCl₃) δ 8.56 (s, 1H), 8.48-8.38 (m, 2H), |
| | | [M + H]⁺ calcd for | 8.34 (d, J = 8.7 Hz, 1H), 7.85-7.77 (m, 2H), 7.59 (d, J = 11.3 |
| | | C₃₁H₂₇F₆N₇O₃S, | Hz, 1H), 7.40 (d, J = 8.5 Hz, 2H), 7.32 (d, J = 8.7 Hz, 1H), 6.88 |
| | | 692.1873; found, | (dd, J = 8.8, 2.8 Hz, 1H), 6.35 (d, J = 2.7 Hz, 1H), 3.99 (s, 2H), |
| | | 692.1884 | 2.96 (s, 6H), 2.57 (q, J = 6.9 Hz, 1H), 1.17 (dd, J = 12.7, 6.8 |
| | | | Hz, 6H) |
| J117 | | HRMS-ESI (m/z) | ¹H NMR (300 MHz, CDCl₃) δ 8.57 (d, J = 0.6 Hz, 1H), 8.46 (d, |
| | | [M + H]⁺ calcd for | J = 8.7 Hz, 1H), 8.41-8.31 (m, 2H), 7.83-7.77 (m, 2H), 7.53 |
| | | C₃₀H₂₄F₆N₆O₄S, | (d, J = 8.3 Hz, 1H), 7.47 (s, 1H), 7.40 (d, J = 7.8 Hz, 3H), 6.91 |
| | | 679.1557; found, | (s, 1H), 4.15 (dd, J = 14.4, 6.7 Hz, 1H), 4.00 (d, J = 1.5 Hz, |
| | | 679.1568 | 2H), 3.14 (d, J = 24.6 Hz, 3H), 2.42 (s, 3H), 1.45-1.31 (m, 3H) |
| J118 | | HRMS-ESI (m/z) | ¹H NMR (300 MHz, CDCl₃) δ 8.57 (s, 1H), 8.47 (d, J = 8.7 Hz, |
| | | [M + H]⁺ calcd for | 1H), 8.41 (s, 1H), 8.35 (d, J = 8.9 Hz, 1H), 7.84-7.78 (m, 2H), |
| | | C₃₁H₂₄F₉N₇O₄S, | 7.55 (s, 1H), 7.40 (d, J = 8.5 Hz, 2H), 7.00 (d, J = 9.1 Hz, 1H), |
| | | 762.154; found, | 6.83 (dd, J = 9.1, 3.1 Hz, 1H), 6.57 (d, J = 3.0 Hz, 1H), 4.21- |
| | | 762.1545 | 4.12 (m, 2H), 3.94 (d, J = 1.7 Hz, 2H), 2.94 (s, 6H), 2.61-2.40 |
| | | | (m, 2H) |
| J119 | 229-232 | HRMS-ESI (m/z) | ¹H NMR (300 MHz, CDCl₃) δ 8.62 (s, 1H), 8.51-8.41 (m, 1H), |
| | | [M + H]⁺ calcd for | 8.07-7.83 (m, 4H), 7.67 (d, J = 5.0 Hz, 2H), 7.54 (dd, J = 8.0, |
| | | C₂₉H₂₅F₄N₆O₃S, | 3.0 Hz, 1H), 7.42 (d, J = 9.4 Hz, 1H), 6.93 (s, 1H), 4.15 (t, J = |
| | | 613.1639; found, | 6.5 Hz, 1H), 4.05-3.94 (m, 2H), 3.15 (d, J = 16.7 Hz, 3H), |
| | | 613.1638 | 2.43 (s, 3H), 1.37 (dd, J = 12.3, 6.4 Hz, 3H); |
| | | | ¹⁹F NMR (471 MHz, CDCl₃) δ −62.83, −131.05, −131.47 |
| J120 | 183-185 | HRMS-ESI (m/z) | ¹H NMR (300 MHz, CDCl₃) δ 8.58 (s, 1H), 8.47 (td, J = 8.4, 3.6 |
| | | [M + H]⁺ calcd for | Hz, 1H), 7.98 (d, J = 8.7 Hz, 1H), 7.89 (dt, J = 12.0, 2.3 Hz, |
| | | C₂₉H₂₅F₄N₆O₄S, | 1H), 7.67 (dd, J = 4.8, 2.3 Hz, 2H), 7.61-7.46 (m, 2H), 7.46- |
| | | 629.1589; found, | 7.33 (m, 2H), 7.32-7.22 (m, 1H), 6.93 (s, 1H), 4.15 (p, J = 6.6 |
| | | 629.1590 | Hz, 1H), 4.03-3.89 (m, 2H), 3.15 (d, J = 16.6 Hz, 3H), 2.43 (s, |
| | | | 3H), 1.37 (dd, J = 12.3, 6.4 Hz, 3H); |
| | | | ¹⁹F NMR (471 MHz, CDCl₃) δ −57.80, −131.12, −131.54. |
| J121 | 208-211 | HRMS-ESI (m/z) | ¹H NMR (300 MHz, CDCl₃) δ 8.58 (s, 1H), 8.50 (t, J = 8.4 Hz, |
| | | [M + H]⁺ calcd for | 1H), 7.98 (d, J = 8.6 Hz, 1H), 7.90 (dd, J = 12.1, 1.8 Hz, 1H), |
| | | C₂₈H₂₀F₇N₆O₄S, | 7.71-7.63 (m, 2H), 7.62-7.47 (m, 2H), 7.31 (d, J = 8.9 Hz, |
| | | 669.1149; found, | 1H), 7.07 (d, J = 2.1 Hz, 1H), 6.99 (d, J = 8.5 Hz, 1H), 4.51- |
| | | 669.1147 | 4.25 (m, 2H), 3.96 (d, J = 3.1 Hz, 2H), 2.40 (s, 3H); ¹⁹F NMR |
| | | | (376 MHz, CDCl₃) δ −131.56, −74.17, −57.80 |
| J122 | 218.5220.5 | HRMS-ESI (m/z) | ¹H NMR (300 MHz, CDCl₃) δ 8.62 (s, 1H), 8.51 (t, J = 8.4 Hz, |
| | | [M + H]⁺ calcd for | 1H), 8.07-7.86 (m, 4H), 7.67 (d, J = 4.8 Hz, 2H), 7.51 (s, 1H), |
| | | C₂₈H₂₀F₇N₆O₃S, | 7.31 (d, J = 9.7 Hz, 1H), 7.07 (s, 1H), 6.99 (d, J = 8.4 Hz, 1H), |
| | | 653.1200; found, | 4.36 (p, J = 8.1 Hz, 2H), 3.96 (d, J = 3.1 Hz, 2H), 2.40 (s, 3H); |
| | | 653.1199 | ¹⁹F NMR (471 MHz, CDCl₃) δ −62.82, −74.17, −131.51 |
| J123 | | HRMS-ESI (m/z) | ¹H NMR (300 MHz, CDCl₃) δ 8.54 (s, 1H), 8.48 (t, J = 8.4 Hz, |
| | | [M + H]⁺ calcd for | 1H), 8.04-7.82 (m, 2H), 7.82-7.71 (m, 2H), 7.54-7.31 (m, |
| | | C₂₉H₂₁F₇N₆O₄S, | 5H), 7.13-6.94 (m, 1H), 4.71-4.44 (m, 2H), 4.07-3.86 (m, |
| | | 683.1306; found, | 2H), 3.71 (qd, J = 8.7, 2.4 Hz, 2H), 2.42 (d, J = 15.4 Hz, 3H); |
| | | 683.1316 | ¹⁹F NMR (376 MHz, CDCl₃) δ −58.02, −73.78, −131.36 |
| J124 | | HRMS-ESI (m/z) | ¹H NMR (300 MHz, CDCl₃) δ 8.54 (s, 1H), 8.47 (td, J = 8.3, 3.7 |
| | | [M + H]⁺ calcd for | Hz, 1H), 8.04-7.83 (m, 2H), 7.82-7.71 (m, 2H), 7.62-7.49 |
| | | C₃₀H₂₆F₄N₆O₄S, | (m, 1H), 7.47-7.32 (m, 4H), 6.91 (d, J = 1.8 Hz, 1H), 4.26 (dd, |
| | | 643.1745; found, | J = 10.9, 6.3 Hz, 1H), 3.99 (d, J = 0.8 Hz, 2H), 3.50-3.12 (m, |
| | | 643.1743 | 2H), 2.42 (s, 3H), 1.37 (dd, J = 13.1, 6.5 Hz, 3H), 1.14 (td, J = |
| | | | 7.0, 4.2 Hz, 3H) |
| J125 | | HRMS-ESI (m/z) | ¹H NMR (300 MHz, CDCl₃) δ 8.53 (d, J = 6.0 Hz, 1H), 8.48 (d, |
| | | [M + H]⁺ calcd for | J = 8.4 Hz, 1H), 8.04-7.83 (m, 2H), 7.83-7.71 (m, 2H), 7.54 |
| | | C₂₉H₂₄F₄N₆O₄S, | (d, J = 3.0 Hz, 1H), 7.46-7.33 (m, 2H), 7.25-7.21 (m, 1H), |
| | | 629.1589; found, | 7.06-6.90 (m, 2H), 4.03-3.86 (m, 4H), 2.37 (s, 3H), 1.73 (p, |
| | | 629.159 | J = 6.9 Hz, 2H), 0.93 (t, J = 7.4 Hz, 3H) |
| J126 | | HRMS-ESI (m/z) | ¹H NMR (300 MHz, CDCl₃) δ 8.54 (s, 1H), 8.20 (d, J = 8.5 Hz, |
| | | [M + H]⁺ calcd for | 1H), 8.03 (d, J = 8.7 Hz, 1H), 7.98 (s, 1H), 7.83-7.75 (m, 2H), |
| | | C₃₀H₂₈F₃N₇O₃S, | 7.38 (d, J = 8.6 Hz, 2H), 7.30 (s, 1H), 7.19 (s, 1H), 6.77 (dd, J = |
| | | 624.1999; found, | 8.6, 2.6 Hz, 1H), 6.31 (d, J = 2.6 Hz, 1H), 3.97 (s, 2H), 2.84 |
| | | 624.2004 | (s, 3H), 2.60 (t, J = 7.0 Hz, 1H), 2.26 (s, 3H), 1.17 (dd, J = |
| | | | 10.3, 6.9 Hz, 6H) |
| J127 | | HRMS-ESI (m/z) | ¹H NMR (300 MHz, CDCl₃) δ 8.54 (s, 1H), 8.20 (d, J = 8.5 Hz, |
| | | [M + H]⁺ calcd for | 1H), 8.07-8.01 (m, 1H), 7.98 (s, 1H), 7.83-7.76 (m, 2H), |
| | | C₃₁H₃₀F₃N₇O₃S, | 7.41-7.35 (m, 2H), 7.26 (d, J = 8.6 Hz, 1H), 7.19 (s, 1H), 6.75 |
| | | 638.2156; found, | (dd, J = 8.6, 2.5 Hz, 1H), 6.30 (d, J = 2.5 Hz, 1H), 3.96 (d, J = |
| | | 638.2164 | 1.0 Hz, 2H), 3.65 (s, 1H), 3.15 (q, J = 7.1 Hz, 2H), 2.59 (p, J = |
| | | | 7.1 Hz, 1H), 2.26 (s, 3H), 1.26 (t, J = 7.1 Hz, 3H), 1.17 (dd, J = |
| | | | 9.4, 6.8 Hz, 6H) |
| J128 | | HRMS-ESI (m/z) | ¹H NMR (300 MHz, CDCl₃) δ 8.54 (s, 1H), 8.20 (d, J = 8.5 Hz, |
| | | [M + H]⁺ calcd for | 1H), 8.03 (d, J = 9.3 Hz, 1H), 7.98 (s, 1H), 7.83-7.76 (m, 2H), |
| | | C₃₂H₃₂F₃N₇O₃S, | 7.38 (d, J = 8.6 Hz, 2H), 7.26 (d, J = 8.6 Hz, 1H), 7.19 (s, 1H), |
| | | 652.2312; found, | 6.75 (dd, J = 8.6, 2.5 Hz, 1H), 6.29 (d, J = 2.5 Hz, 1H), 3.96 (d, |
| | | 652.232 | J = 1.0 Hz, 2H), 3.72 (s, 1H), 3.07 (t, J = 7.1 Hz, 2H), 2.59 (p, J = |
| | | | 6.9 Hz, 1H), 2.26 (s, 3H), 1.66 (dt, J = 14.5, 7.3 Hz, 2H), 1.17 |
| | | | (dd, J = 9.4, 6.8 Hz, 6H), 1.00 (t, J = 7.3 Hz, 3H) |
| J129 | | HRMS-ESI (m/z) | ¹H NMR (300 MHz, CDCl₃) δ 8.54 (s, 1H), 8.19 (d, J = 8.5 Hz, |
| | | [M + H]⁺ calcd for | 1H), 8.04 (d, J = 8.6 Hz, 1H), 7.98 (s, 1H), 7.83-7.76 (m, 2H), |
| | | C₃₃H₃₄F₃N₇O₃S, | 7.43-7.35 (m, 2H), 7.29 (d, J = 8.8 Hz, 1H), 7.21 (s, 1H), 6.81 |
| | | 666.2469; found, | (dd, J = 8.8, 2.8 Hz, 1H), 6.30 (d, J = 2.7 Hz, 1H), 4.00-3.94 |
| | | 666.2481 | (m, 2H), 3.33 (dq, J = 14.7, 7.4 Hz, 4H), 2.59 (p, J = 5.8 Hz, |
| | | | 1H), 2.26 (s, 3H), 1.17 (t, J = 7.0 Hz, 12H) |
| J130 | | HRMS-ESI (m/z) | ¹H NMR (300 MHz, CDCl₃) δ 8.54 (s, 1H), 8.19 (d, J = 8.5 Hz, |
| | | [M + H]⁺ calcd for | 1H), 8.04 (d, J = 8.5 Hz, 1H), 7.99 (s, 1H), 7.83-7.77 (m, 2H), |
| | | C₃₅H₃₈F₃N₇O₃S, | 7.38 (d, J = 8.6 Hz, 2H), 7.29 (s, 1H), 7.20 (s, 1H), 6.77 (dd, J = |
| | | 694.2782; found, | 8.8, 2.8 Hz, 1H), 6.25 (d, J = 2.7 Hz, 1H), 3.97 (s, 2H), 3.20 |
| | | 694.2789 | (hept, J = 7.3 Hz, 4H), 2.58 (p, J = 7.1 Hz, 1H), 2.26 (s, 3H), |
| | | | 1.62 (q, J = 7.7 Hz, 4H), 1.17 (t, J = 7.1 Hz, 6H), 0.97-0.88 |
| | | | (m, 6H) |
| J131 | 208-210 | HRMS-ESI (m/z) | ¹H NMR (500 MHz, CDCl₃) δ 8.61 (s, 1H), 8.19 (d, J = 8.5 Hz, |
| | | [M + H]⁺ calcd for | 1H), 8.08-8.02 (m, 2H), 8.00 (s, 1H), 7.98-7.91 (m, 1H), |
| | | C₃₀H₂₈F₃N₆O₂S, | 7.71-7.62 (m, 2H), 7.38 (d, J = 8.1 Hz, 1H), 7.35-7.29 (m, |
| | | 593.1941; found, | 1H), 7.13 (s, 1H), 6.92 (d, J = 1.8 Hz, 1H), 3.97 (d, J = 1.4 Hz, |
| | | 593.1941 | 2H), 2.69 (p, J = 6.9 Hz, 1H), 2.39 (s, 3H), 2.26 (s, 3H), 1.30- |
| | | | 1.20 (m, 3H), 1.18 (d, J = 6.9 Hz, 3H); |
| | | | ¹⁹F NMR (471 MHz, CDCl₃) δ −62.80 |
| J132 | 219-223 | HRMS-ESI (m/z) | ¹H NMR (500 MHz, CDCl₃) δ 8.61 (s, 1H), 8.20 (d, J = 8.5 Hz, |
| | | [M + H]⁺ calcd for | 1H), 8.06 (dd, J = 7.2, 3.2 Hz, 1H), 8.05 (s, 1H), 8.00 (d, J = |
| | | C₃₀H₂₈F₃N₆O₃S, | 2.1 Hz, 1H), 7.95 (ddd, J = 5.7, 3.9, 2.2 Hz, 1H), 7.70-7.62 |
| | | 609.1890; found, | (m, 2H), 7.39 (d, J = 8.7 Hz, 1H), 7.14 (s, 1H), 7.07 (dd, J = |
| | | 609.1895 | 8.7, 2.7 Hz, 1H), 6.63 (d, J = 2.7 Hz, 1H), 3.98 (d, J = 1.4 Hz, |
| | | | 2H), 3.83 (s, 3H), 2.65 (h, J = 6.8 Hz, 1H), 2.27 (s, 3H), 1.19 |
| | | | (dd, J = 15.8, 6.8 Hz, 6H); |
| | | | ¹⁹F NMR (471 MHz, CDCl₃) δ −62.79 |
| J133 | | HRMS-ESI (m/z) | ¹H NMR (500 MHz, CDCl₃) δ 8.61 (s, 1H), 8.20 (d, J = 8.5 Hz, |
| | | [M + H]⁺ calcd for | 1H), 8.05 (q, J = 3.8, 2.9 Hz, 2H), 8.03-7.98 (m, 1H), 7.98- |
| | | C₃₁H₃₁F₃N₇O₂S, | 7.91 (m, 1H), 7.70-7.62 (m, 2H), 7.33 (d, J = 8.8 Hz, 1H), |
| | | 622.2207; found, | 7.20 (s, 1H), 6.89 (dd, J = 8.8, 2.7 Hz, 1H), 6.38 (d, J = 2.7 Hz, |
| | | 622.2216 | 1H), 3.97 (d, J = 1.1 Hz, 2H), 2.96 (s, 6H), 2.60 (h, J = 6.7 Hz, |
| | | | 1H), 2.27 (s, 3H), 1.20 (d, J = 6.8 Hz, 3H), 1.16 (d, J = 6.9 Hz, |
| | | | 3H); ¹⁹F NMR (471 MHz, CDCl₃) δ −62.79 |
| J134 | 144-150 | HRMS-ESI (m/z) | ¹H NMR (500 MHz, CDCl₃) δ 8.61 (s, 1H), 8.21 (d, J = 8.5 Hz, |
| | | [M + H]⁺ calcd for | 1H), 8.05 (q, J = 4.9, 3.7 Hz, 2H), 8.00 (d, J = 2.1 Hz, 1H), 7.98- |
| | | C₃₀H₂₅F₆N₆O₃S, | 7.91 (m, 1H), 7.70-7.62 (m, 2H), 7.28 (dd, J = 8.4, 2.0 Hz, |
| | | 663.1608; found, | 1H), 7.11 (s, 1H), 7.05 (d, J = 2.2 Hz, 1H), 6.97 (d, J = 8.4 Hz, |
| | | 663.1611 | 1H), 4.23 (td, J = 6.3, 1.7 Hz, 2H), 3.92 (d, J = 2.7 Hz, 2H), |
| | | | 2.55 (tddd, J = 12.4, 8.3, 6.5, 3.4 Hz, 2H), 2.38 (s, 3H), 2.28 (s, |
| | | | 3H); ¹⁹F NMR (471 MHz, CDCl₃) δ −62.79, −64.57 |
| J135 | | HRMS-ESI (m/z) | ¹H NMR (500 MHz, CDCl₃) δ 8.57 (s, 1H), 8.18 (d, J = 8.5 Hz, |
| | | [M + H]⁺ calcd for | 1H), 8.04 (dd, J = 8.4, 2.0 Hz, 1H), 7.99 (d, J = 1.9 Hz, 1H), |
| | | C₃₀H₂₈F₃N₆O₃S, | 7.71-7.65 (m, 2H), 7.55 (t, J = 8.5 Hz, 1H), 7.38 (d, J = 8.1 |
| | | 609.1890; found, | Hz, 1H), 7.35-7.28 (m, 1H), 7.29-7.22 (m, 1H), 7.14 (s, 1H), |
| | | 609.1892 | 6.92 (t, J = 1.2 Hz, 1H), 3.97 (d, J = 1.5 Hz, 2H), 2.68 (h, J = |
| | | | 6.9 Hz, 1H), 2.39 (s, 3H), 2.26 (s, 3H), 1.23 (d, J = 6.8 Hz, 3H), |
| | | | 1.18 (d, J = 6.9 Hz, 3H); |
| | | | ¹⁹F NMR (471 MHz, CDCl₃) δ −57.78 |
| J136 | | HRMS-ESI (m/z) | ¹H NMR (500 MHz, CDCl₃) δ 8.57 (s, 1H), 8.19 (d, J = 8.5 Hz, |
| | | [M + H]⁺ calcd for | 1H), 8.04 (dd, J = 8.5, 2.0 Hz, 1H), 8.02-7.97 (m, 1H), 7.71- |
| | | C₃₀H₂₈F₃N₆O₄S, | 7.65 (m, 2H), 7.55 (t, J = 8.5 Hz, 1H), 7.39 (d, J = 8.7 Hz, 1H), |
| | | 625.1839; found, | 7.26 (s, 1H), 7.14 (s, 1H), 7.07 (dd, J = 8.7, 2.7 Hz, 1H), 6.63 |
| | | 625.1842 | (d, J = 2.7 Hz, 1H), 3.98 (d, J = 1.4 Hz, 2H), 3.82 (s, 3H), 2.65 |
| | | | (p, J = 6.8 Hz, 1H), 2.26 (s, 3H), 1.19 (dd, J = 15.6, 6.8 Hz, |
| | | | 6H); ¹⁹F NMR (471 MHz, CDCl₃) δ −57.78 |
| J137 | | HRMS-ESI (m/z) | ¹H NMR (500 MHz, CDCl₃) δ 8.57 (s, 1H), 8.19 (d, J = 8.4 Hz, |
| | | [M + H]⁺ calcd for | 1H), 8.04 (dd, J = 8.6, 2.0 Hz, 1H), 7.99 (d, J = 2.2 Hz, 1H), |
| | | C₃₁H₃₁F₃N₇O₃S, | 7.71-7.65 (m, 2H), 7.55 (t, J = 8.5 Hz, 1H), 7.33 (d, J = 8.8 |
| | | 638.2156; found, | Hz, 1H), 7.29-7.22 (m, 1H), 7.20 (s, 1H), 6.88 (dd, J = 8.8, |
| | | 638.2163 | 2.7 Hz, 1H), 6.38 (d, J = 2.7 Hz, 1H), 3.97 (d, J = 1.0 Hz, 2H), |
| | | | 2.96 (s, 6H), 2.61 (p, J = 6.8 Hz, 1H), 2.26 (s, 3H), 1.20 (d, J = |
| | | | 6.8 Hz, 3H), 1.16 (d, J = 6.9 Hz, 3H); |
| | | | ¹⁹F NMR (471 MHz, CDCl₃) δ −57.78 |
| J138 | | HRMS-ESI (m/z) | ¹H NMR (500 MHz, CDCl₃) δ 8.57 (s, 1H), 8.20 (d, J = 8.5 Hz, |
| | | [M + H]⁺ calcd for | 1H), 8.07-8.01 (m, 1H), 7.99 (d, J = 1.7 Hz, 1H), 7.72-7.65 |
| | | C₃₀H₂₅F₆N₆O₄S, | (m, 2H), 7.55 (t, J = 8.5 Hz, 1H), 7.31-7.22 (m, 2H), 7.11 (s, |
| | | 679.1557; found, | 1H), 7.05 (d, J = 2.5 Hz, 1H), 6.97 (d, J = 8.5 Hz, 1H), 4.23 (td, |
| | | 679.1562 | J = 6.3, 1.7 Hz, 2H), 3.92 (d, J = 2.7 Hz, 2H), 2.62-2.51 (m, |
| | | | 2H), 2.38 (s, 3H), 2.28 (s, 3H); |
| | | | ¹⁹F NMR (471 MHz, CDCl₃) δ −57.78, −64.58 |
| J139 | | HRMS-ESI (m/z) | ¹H NMR (300 MHz, CDCl₃) δ 8.54 (s, 1H), 8.19 (d, J = 8.5 Hz, |
| | | [M + H]⁺ calcd for | 1H), 8.11-7.93 (m, 2H), 7.86-7.71 (m, 2H), 7.38 (td, J = |
| | | C₃₀H₂₄F₆N₆O₄S, | 13.0, 12.1, 7.8 Hz, 4H), 7.05 (s, 2H), 4.63 (d, J = 12.4 Hz, 1H), |
| | | 679.1557; found, | 4.51 (d, J = 12.5 Hz, 1H), 3.94 (d, J = 1.2 Hz, 2H), 3.73 (q, J = |
| | | 679.1559 | 8.7 Hz, 2H), 2.45 (s, 3H), 2.26 (s, 3H) |
| J140 | 233-238 | HRMS-ESI (m/z) | ¹H NMR (500 MHz, CDCl₃) δ 8.61 (s, 1H), 8.49 (t, J = 8.3 Hz, |
| | | [M + H]⁺ calcd for | 1H), 8.06-8.01 (m, 1H), 8.01-7.87 (m, 3H), 7.71-7.63 (m, |
| | | C₂₉H₂₅F₄N₆O₂S, | 2H), 7.54 (d, J = 2.9 Hz, 1H), 7.39 (d, J = 8.1 Hz, 1H), 7.33 (dd, |
| | | 597.169; found, | J = 8.5, 1.6 Hz, 1H), 6.90 (t, J = 1.2 Hz, 1H), 3.99 (d, J = 3.5 |
| | | 597.1692 | Hz, 2H), 2.67 (h, J = 6.7 Hz, 1H), 2.39 (s, 3H), 1.20 (dd, J = |
| | | | 11.6, 6.8 Hz, 6H); ¹⁹F NMR (471 MHz, CDCl₃) δ −62.82, −130.97 |
| J141 | | HRMS-ESI (m/z) | ¹H NMR (300 MHz, CDCl₃) δ 8.54 (s, 1H), 8.18 (dd, J = 8.5, 3.2 |
| | | [M + H]⁺ calcd for | Hz, 1H), 8.09-7.93 (m, 2H), 7.86-7.70 (m, 2H), 7.64-7.49 |
| | | C₃₁H₂₉F₃N₆O₄S, | (m, 1H), 7.38 (d, J = 8.4 Hz, 3H), 7.11-6.86 (m, 2H), 4.28 (dd, |
| | | 639.1996; found, | J = 14.6, 8.1 Hz, 1H), 3.97 (s, 2H), 3.49-3.12 (m, 2H), 2.42 (s, |
| | | 639.2001 | 3H), 2.23 (d, J = 11.2 Hz, 3H), 1.38 (dd, J = 20.0, 6.4 Hz, 3H), |
| | | | 1.21-1.07 (m, 3H) |
| J142 | 200-202 | HRMS-ESI (m/z) | ¹H NMR (500 MHz, CDCl₃) δ 8.62 (s, 1H), 8.50 (t, J = 8.3 Hz, |
| | | [M + H]⁺ calcd for | 1H), 8.06-8.01 (m, 1H), 8.02-7.94 (m, 1H), 7.98-7.87 (m, |
| | | C₂₉H₂₂F₇N₆O₃S, | 2H), 7.71-7.63 (m, 2H), 7.52 (d, J = 3.0 Hz, 1H), 7.32-7.25 |
| | | 667.1357; found, | (m, 1H), 7.05-7.00 (m, 1H), 6.97 (d, J = 8.4 Hz, 1H), 4.28- |
| | | 667.1361 | 4.16 (m, 2H), 3.93 (d, J = 2.5 Hz, 2H), 2.60-2.49 (m, 2H), |
| | | | 2.38 (s, 3H); ¹⁹F NMR (471 MHz, CDCl₃) δ −62.82, −64.60, −131.42 |
| J143 | 162-169 | HRMS-ESI (m/z) | ¹H NMR (500 MHz, CDCl₃) δ 8.57 (s, 1H), 8.48 (t, J = 8.4 Hz, |
| | | [M + H]⁺ calcd for | 1H), 7.97 (dd, J = 8.7, 1.7 Hz, 1H), 7.89 (dd, J = 11.9, 1.9 Hz, |
| | | C₂₉H₂₅F₄N₆O₃S, | 1H), 7.72-7.64 (m, 2H), 7.60-7.52 (m, 2H), 7.39 (d, J = 8.0 |
| | | 613.1639; found, | Hz, 1H), 7.33 (dd, J = 8.2, 1.8 Hz, 1H), 7.26 (s, 1H), 6.90 (t, J = |
| | | 613.1639 | 1.2 Hz, 1H), 3.99 (d, J = 3.5 Hz, 2H), 2.66 (p, J = 6.8 Hz, 1H), |
| | | | 2.39 (s, 3H), 1.20 (dd, J = 11.6, 6.8 Hz, 6H); ¹⁹F NMR (471 |
| | | | MHz, CDCl₃) δ −57.80, −130.99 |
| J144 | | HRMS-ESI (m/z) | ¹H NMR (500 MHz, CDCl₃) δ 8.45 (d, J = 11.9 Hz, 2H), 7.96 (d, |
| | | [M + H]⁺ calcd for | J = 8.5 Hz, 1H), 7.88 (dd, J = 12.1, 1.9 Hz, 1H), 7.66-7.58 (m, |
| | | C₂₉H₂₈FN₆O₃S, | 2H), 7.53 (d, J = 2.9 Hz, 1H), 7.38 (d, J = 8.1 Hz, 1H), 7.36- |
| | | 559.1922; found, | 7.29 (m, 1H), 7.06-6.98 (m, 2H), 6.92-6.88 (m, 1H), 3.99 (d, |
| | | 559.1922 | J = 3.5 Hz, 2H), 3.87 (s, 3H), 2.65 (h, J = 6.9 Hz, 1H), 2.39 (s, |
| | | | 3H), 1.19 (dd, J = 11.5, 6.9 Hz, 6H); |
| | | | ¹⁹F NMR (471 MHz, CDCl₃) δ −131.19 |
| J145 | 201-206 | HRMS-ESI (m/z) | ¹H NMR (500 MHz, CDCl₃) δ 8.46 (t, J = 8.4 Hz, 1H), 8.44 (s, |
| | | [M + H]⁺ calcd for | 1H), 7.96 (d, J = 8.8 Hz, 1H), 7.88 (dd, J = 12.0, 1.9 Hz, 1H), |
| | | C₂₉H₂₈FN₆O₄S, | 7.66-7.58 (m, 2H), 7.53 (d, J = 3.0 Hz, 1H), 7.39 (d, J = 8.8 |
| | | 575.1871; found, | Hz, 1H), 7.08 (dd, J = 8.7, 2.7 Hz, 1H), 7.08-6.98 (m, 2H), |
| | | 575.1874 | 6.61 (t, J = 2.8 Hz, 1H), 3.99 (d, J = 2.9 Hz, 2H), 3.87 (s, 3H), |
| | | | 3.82 (d, J = 1.8 Hz, 3H), 2.63 (p, J = 6.8 Hz, 1H), 1.18 (t, J = |
| | | | 7.3 Hz, 6H); ¹⁹F NMR (471 MHz, CDCl₃) δ −131.19 |
| J146 | 183-187 | HRMS-ESI (m/z) | ¹H NMR (500 MHz, CDCl₃) δ 8.47 (t, J = 8.3 Hz, 1H), 8.44 (s, |
| | | [M + H]⁺ calcd for | 1H), 7.99-7.93 (m, 1H), 7.88 (dd, J = 12.0, 1.8 Hz, 1H), 7.64- |
| | | C₃₀H₃₁FN₇O₃S, | 7.60 (m, 2H), 7.59 (d, J = 2.9 Hz, 1H), 7.33 (d, J = 8.8 Hz, |
| | | 588.2188; found, | 1H), 7.06-6.98 (m, 2H), 6.89 (dd, J = 8.8, 2.7 Hz, 1H), 6.36 |
| | | 588.2197 | (d, J = 2.7 Hz, 1H), 3.98 (d, J = 2.6 Hz, 2H), 3.87 (s, 3H), 2.96 |
| | | | (d, J = 1.7 Hz, 6H), 2.58 (p, J = 6.9 Hz, 1H), 1.17 (dd, J = 11.4, |
| | | | 6.9 Hz, 6H); ¹⁹F NMR (471 MHz, CDCl₃) δ −130.90 |
| J147 | | HRMS-ESI (m/z) | ¹H NMR (500 MHz, CDCl₃) δ 8.48 (t, J = 8.4 Hz, 1H), 8.44 (s, |
| | | [M + H]⁺ calcd for | 1H), 7.96 (d, J = 8.7 Hz, 1H), 7.88 (dd, J = 12.2, 1.9 Hz, 1H), |
| | | C₂₉H₂₅F₄N₆O₄S, | 7.66-7.58 (m, 2H), 7.51 (d, J = 3.0 Hz, 1H), 7.32-7.25 (m, |
| | | 629.1589; found, | 1H), 7.06-6.99 (m, 3H), 6.97 (d, J = 8.4 Hz, 1H), 4.22 (tq, J = |
| | | 629.1591 | 8.0, 3.7 Hz, 2H), 3.93 (d, J = 2.5 Hz, 2H), 3.87 (s, 3H), 2.54 |
| | | | (dtd, J = 10.2, 6.3, 3.8 Hz, 2H), 2.38 (s, 3H); |
| | | | ¹⁹F NMR (471 MHz, CDCl₃) δ −64.60, −131.65 |
| J148 | 231-233 | HRMS-ESI (m/z) | ¹H NMR (500 MHz, CDCl₃) δ 8.50 (s, 1H), 8.46 (t, J = 8.4 Hz, |
| | | [M + H]⁺ calcd for | 1H), 7.97 (d, J = 9.1 Hz, 1H), 7.89 (dd, J = 12.1, 1.8 Hz, 1H), |
| | | C₂₉H₂₈FN₆O₂S, | 7.64-7.56 (m, 2H), 7.53 (d, J = 3.0 Hz, 1H), 7.38 (d, J = 8.1 |
| | | 543.1973; found, | Hz, 1H), 7.36-7.28 (m, 3H), 6.93-6.88 (m, 1H), 3.99 (d, J = |
| | | 543.1974 | 3.5 Hz, 2H), 2.66 (p, J = 6.9 Hz, 1H), 2.42 (s, 3H), 2.39 (s, 3H), |
| | | | 1.19 (dd, J = 11.6, 6.8 Hz, 6H); |
| | | | ¹⁹F NMR (471 MHz, CDCl₃) δ −131.19 |
| J149 | 247-249 | HRMS-ESI (m/z) | ¹H NMR (500 MHz, CDCl₃) δ 8.50 (s, 1H), 8.46 (t, J = 8.3 Hz, |
| | | [M + H]⁺ calcd for | 1H), 7.97 (dd, J = 9.0, 1.9 Hz, 1H), 7.89 (dd, J = 12.1, 1.8 Hz, |
| | | C₂₉H₂₈FN₆O₃S, | 1H), 7.64-7.56 (m, 2H), 7.54 (d, J = 3.0 Hz, 1H), 7.39 (d, J = |
| | | 559.1922; found, | 8.8 Hz, 1H), 7.35-7.28 (m, 2H), 7.08 (dd, J = 8.8, 2.7 Hz, 1H), |
| | | 559.1925 | 6.61 (d, J = 2.7 Hz, 1H), 3.99 (d, J = 3.0 Hz, 2H), 3.83 (s, 3H), |
| | | | 2.63 (p, J = 6.9 Hz, 1H), 2.42 (s, 3H), 1.22-1.14 (m, 6H); ¹⁹F |
| | | | NMR (471 MHz, CDCl₃) δ −131.18 |
| J150 | | HRMS-ESI (m/z) | ¹H NMR (500 MHz, CDCl₃) δ 8.50 (s, 1H), 8.47 (t, J = 8.4 Hz, |
| | | [M + H]⁺ calcd for | 1H), 8.00-7.94 (m, 1H), 7.88 (dd, J = 12.1, 1.9 Hz, 1H), 7.60 |
| | | C₃₀H₃₁FN₇O₂S, | (dd, J = 8.9, 2.4 Hz, 3H), 7.32 (t, J = 8.3 Hz, 3H), 6.89 (dd, J = |
| | | 572.2238; found, | 8.8, 2.8 Hz, 1H), 6.36 (d, J = 2.7 Hz, 1H), 3.98 (d, J = 2.6 Hz, |
| | | 572.2241 | 2H), 2.97 (s, 6H), 2.58 (p, J = 6.8 Hz, 1H), 2.42 (s, 3H), 1.17 |
| | | | (dd, J = 11.4, 6.9 Hz, 6H); |
| | | | ¹⁹F NMR (471 MHz, CDCl₃) δ −130.90 |
| J151 | 127-131 | HRMS-ESI (m/z) | ¹H NMR (500 MHz, CDCl₃) δ 8.52-8.44 (m, 2H), 7.97 (dd, J = |
| | | [M + H]⁺ calcd for | 8.6, 1.7 Hz, 1H), 7.89 (dd, J = 12.1, 1.9 Hz, 1H), 7.64-7.56 |
| | | C₂₉H₂₅F₄N₆O₃S, | (m, 2H), 7.52 (d, J = 3.0 Hz, 1H), 7.34-7.25 (m, 3H), 7.02 (d, |
| | | 613.1639; found, | J = 2.2 Hz, 1H), 6.96 (d, J = 8.5 Hz, 1H), 4.27-4.17 (m, 2H), |
| | | 613.1646 | 3.93 (d, J = 2.5 Hz, 2H), 2.61-2.47 (m, 2H), 2.42 (s, 3H), 2.38 |
| | | | (s, 3H); ¹⁹F NMR (471 MHz, CDCl₃) δ −64.60, −131.63 |
| J152 | 223-225 | HRMS-ESI (m/z) | ¹H NMR (500 MHz, CDCl₃) δ 8.62 (s, 1H), 8.49 (t, J = 8.3 Hz, |
| | | [M + H]⁺ calcd for | 1H), 8.04 (dd, J = 2.1, 1.2 Hz, 1H), 8.02-7.87 (m, 3H), 7.71- |
| | | C₂₉H₂₅F₄N₆O₃S, | 7.63 (m, 2H), 7.55 (d, J = 2.9 Hz, 1H), 7.40 (d, J = 8.8 Hz, 1H), |
| | | 613.1639; found, | 7.08 (dd, J = 8.7, 2.7 Hz, 1H), 6.61 (d, J = 2.7 Hz, 1H), 4.00 (d, |
| | | 613.1641 | J = 3.0 Hz, 2H), 3.83 (s, 3H), 2.63 (p, J = 6.8 Hz, 1H), 1.19 (dd, |
| | | | J = 7.8, 6.9 Hz, 6H); ¹⁹F NMR (471 MHz, CDCl₃) |
| | | | δ −62.82, −130.96 |
| J153 | | HRMS-ESI (m/z) | ¹H NMR (500 MHz, CDCl₃) δ 8.61 (s, 1H), 8.50 (t, J = 8.3 Hz, |
| | | [M + H]⁺ calcd for | 1H), 8.04 (s, 1H), 8.01-7.87 (m, 3H), 7.71-7.63 (m, 2H), |
| | | C₃₀H₂₈F₄N₇O₂S, | 7.60 (s, 1H), 7.33 (d, J = 8.8 Hz, 1H), 6.89 (dd, J = 8.8, 2.7 Hz, |
| | | 626.1956; found, | 1H), 6.36 (d, J = 2.7 Hz, 1H), 3.99 (d, J = 2.6 Hz, 2H), 2.97 (s, |
| | | 626.1958 | 6H), 2.59 (p, J = 6.9 Hz, 1H), 1.17 (dd, J = 11.6, 6.8 Hz, 6H); |
| | | | ¹⁹F NMR (471 MHz, CDCl₃) δ −62.82, −130.68 |
| J154 | 211-212 | HRMS-ESI (m/z) | ¹H NMR (300 MHz, CDCl₃) δ 8.58 (s, 1H), 8.49 (t, J = 8.4 Hz, |
| | | [M + H]⁺ calcd for | 1H), 7.98 (d, J = 8.7 Hz, 1H), 7.90 (dd, J = 12.0, 1.8 Hz, 1H), |
| | | C₂₉H₂₅F₄N₆O₄S, | 7.72-7.63 (m, 2H), 7.60-7.50 (m, 2H), 7.40 (d, J = 8.8 Hz, |
| | | 629.1589; found, | 1H), 7.29-7.26 (m, 1H), 7.08 (dd, J = 8.8, 2.8 Hz, 1H), 6.62 |
| | | 629.1590 | (d, J = 2.7 Hz, 1H), 4.00 (d, J = 1.2 Hz, 2H), 3.83 (s, 3H), 2.63 |
| | | | (p, J = 6.7 Hz, 1H), 1.19 (dd, J = 6.9, 4.3 Hz, 6H); ¹⁹F NMR |
| | | | (471 MHz, CDCl₃) δ −57.80, −130.99 |
| J155 | | HRMS-ESI (m/z) | ¹H NMR (300 MHz, CDCl₃) δ 8.58 (s, 1H), 8.49 (t, J = 8.3 Hz, |
| | | [M + H]⁺ calcd for | 1H), 7.97 (d, J = 8.6 Hz, 1H), 7.89 (dd, J = 12.0, 1.8 Hz, 1H), |
| | | C₃₀H₂₈F₄N₇O₃S, | 7.74-7.64 (m, 2H), 7.61-7.48 (m, 2H), 7.33 (d, J = 8.8 Hz, |
| | | 642.1905; found, | 1H), 7.28-7.26 (m, 1H), 6.90 (dd, J = 8.8, 2.7 Hz, 1H), 6.36 |
| | | 642.1912 | (d, J = 2.7 Hz, 1H), 3.99 (d, J = 1.0 Hz, 2H), 2.97 (s, 6H), 2.58 |
| | | | (p, J = 6.7 Hz, 1H), 1.17 (t, J = 6.8 Hz, 6H); |
| | | | ¹⁹F NMR (471 MHz, CDCl₃) δ −57.79, −130.70 |
| J156 | | HRMS-ESI (m/z) | ¹H NMR (300 MHz, CDCl₃) δ 8.58 (s, 1H), 8.50 (t, J = 8.4 Hz, |
| | | [M + H]⁺ calcd for | 1H), 7.98 (d, J = 7.8 Hz, 1H), 7.90 (d, J = 12.1 Hz, 1H), 7.67 (s, |
| | | C₂₉H₂₂F₇N₆O₄S, | 2H), 7.62-7.48 (m, 2H), 7.30-7.25(m, 1H), 7.03 (s, 1H), 6.97 |
| | | 683.1306; found, | (d, J = 8.4 Hz, 1H), 4.22 (t, J = 6.1 Hz, 2H), 3.94 (s, 2H), 2.67- |
| | | 683.1303 | 2.43 (m, 2H), 2.39 (s, 3H); |
| | | | ¹⁹F NMR (471 MHz, CDCl₃) δ −57.80, −64.60, −131.45 |
| J157 | | HRMS-ESI (m/z) | ¹H NMR (300 MHz, CDCl₃) δ 8.54 (s, 1H), 8.17 (d, J = 8.5 Hz, |
| | | [M + H]⁺ calcd for | 1H), 8.03 (dd, J = 8.5, 2.0 Hz, 1H), 7.99 (d, J = 2.0 Hz, 1H), |
| | | C₃₁H₃₀F₃N₇O₃S, | 7.83-7.76 (m, 2H), 7.42-7.35 (m, 3H), 7.28 (d, J = 8.7 Hz, |
| | | 638.2156; found, | 1H), 6.75 (dd, J = 8.6, 2.5 Hz, 1H), 6.32 (d, J = 2.5 Hz, 1H), |
| | | 638.2167 | 3.97 (s, 2H), 3.90 (s, 1H), 2.78 (s, 3H), 2.64-2.51 (m, 3H), |
| | | | 1.24-1.14 (m, 9H) |
| J158 | | HRMS-ESI (m/z) | ¹H NMR (300 MHz, CDCl₃) δ 8.54 (s, 1H), 8.18 (d, J = 8.5 Hz, |
| | | [M + H]⁺ calcd for | 1H), 8.07-8.02 (m, 1H), 8.00 (s, 1H), 7.84-7.76 (m, 2H), |
| | | C₃₂H₃₂F₃N₇O₃S, | 7.38 (d, J = 8.5 Hz, 2H), 7.26 (d, J = 9.2 Hz, 2H), 6.76 (dd, J = |
| | | 652.2312; found, | 8.6, 2.5 Hz, 1H), 6.30 (d, J = 2.5 Hz, 1H), 3.96 (s, 2H), 3.64 (s, |
| | | 652.2318 | 1H), 3.15 (q, J = 7.2 Hz, 2H), 2.60 (q, J = 7.6 Hz, 3H), 1.26 (d, |
| | | | J = 1.1 Hz, 6H), 1.22-1.13 (m, 6H) |
| J159 | | HRMS-ESI (m/z) | ¹H NMR (300 MHz, CDCl₃) δ 8.54 (s, 1H), 8.18 (d, J = 8.5 Hz, |
| | | [M + H]⁺ calcd for | 1H), 8.04 (dd, J = 8.4, 2.0 Hz, 1H), 8.00 (d, J = 2.0 Hz, 1H), |
| | | C₃₃H₃₄F₃N₇O₃S, | 7.84-7.76 (m, 2H), 7.38 (dt, J = 7.9, 1.0 Hz, 2H), 7.30-7.22 |
| | | 666.2469; found, | (m, 2H), 6.75 (dd, J = 8.6, 2.5 Hz, 1H), 6.30 (d, J = 2.5 Hz, 1H), |
| | | 666.2475 | 3.96 (d, J = 0.8 Hz, 2H), 3.72 (s, 1H), 3.07 (t, J = 7.1 Hz, 2H), |
| | | | 2.60 (q, J = 7.5 Hz, 3H), 1.64 (q, J = 7.2 Hz, 2H), 1.23 (t, J = |
| | | | 7.6 Hz, 3H), 1.17 (dd, J = 9.3, 6.9 Hz, 6H), 1.00 (t, J = 7.4 Hz, |
| | | | 3H) |
| J160 | | HRMS-ESI (m/z) | ¹H NMR (300 MHz, CDCl₃) δ 8.54 (s, 1H), 8.17 (d, J = 8.5 Hz, |
| | | [M + H]⁺ calcd for | 1H), 8.07-8.02 (m, 1H), 8.00 (d, J = 2.0 Hz, 1H), 7.84-7.76 |
| | | C₃₄H₃₆F₃N₇O₃S, | (m, 2H), 7.41-7.35 (m, 2H), 7.29 (d, J = 8.9 Hz, 2H), 6.82 (dd, |
| | | 680.2625; found, | J = 8.8, 2.8 Hz, 1H), 6.30 (d, J = 2.7 Hz, 1H), 3.97 (d, J = 0.8 |
| | | 680.2627 | Hz, 2H), 3.35 (hept, J = 7.5 Hz, 4H), 2.60 (q, J = 7.7 Hz, 3H), |
| | | | 1.24-1.14 (m, 15H) |
| J161 | | HRMS-ESI (m/z) | ¹H NMR (300 MHz, CDCl₃) δ 8.54 (s, 1H), 8.17 (d, J = 8.5 Hz, |
| | | [M + H]⁺ calcd for | 1H), 8.04 (dd, J = 8.5, 2.0 Hz, 1H), 8.00 (d, J = 1.9 Hz, 1H), |
| | | C₃₆H₄₀F₃N₇O₃S, | 7.84-7.76 (m, 2H), 7.38 (dd, J = 9.1, 1.0 Hz, 2H), 7.30-7.27 |
| | | 708.2938; found, | (m, 2H), 6.77 (dd, J = 8.8, 2.8 Hz, 1H), 6.25 (d, J = 2.7 Hz, 1H), |
| | | 708.2946 | 3.97 (s, 2H), 3.20 (hept, J = 7.5 Hz, 4H), 2.59 (p, J = 7.3 Hz, |
| | | | 3H), 1.63 (s, 4H), 1.29-1.13 (m, 10H), 0.99-0.85 (m, 9H) |
| J162 | | HRMS-ESI (m/z) | ¹H NMR (300 MHz, CDCl₃) δ 8.54 (s, 1H), 8.48 (t, J = 8.3 Hz, |
| | | [M + H]⁺ calcd for | 1H), 8.03-7.84 (m, 2H), 7.84-7.73 (m, 2H), 7.56 (d, J = 3.1 |
| | | C₃₀H₂₆F₅N₇O₃S, | Hz, 1H), 7.43-7.33 (m, 2H), 7.09 (d, J = 14.2 Hz, 1H), 6.53 (d, |
| | | 660.1811; found, | J = 8.4 Hz, 1H), 3.99 (d, J = 1.0 Hz, 2H), 2.87 (d, J = 0.8 Hz, |
| | | 660.1822 | 6H), 2.68-2.47 (m, 1H), 1.17 (dd, J = 6.9, 4.8 Hz, 6H) |
| J163 | | HRMS-ESI (m/z) | ¹H NMR (300 MHz, CDCl₃) δ 8.54 (s, 1H), 8.46 (t, J = 8.4 Hz, |
| | | [M + H]⁺ calcd for | 1H), 8.20 (s, 1H), 7.97 (d, J = 8.6 Hz, 1H), 7.88 (dd, J = 12.0, |
| | | C₂₆H₁₇F₄N₇O₅S, | 1.8 Hz, 1H), 7.82-7.75 (m, 2H), 7.50 (d, J = 8.3 Hz, 1H), 7.43 |
| | | 616.1021; found, | (d, J = 3.0 Hz, 1H), 7.42-7.36 (m, 2H), 7.22 (s, 1H), 4.15- |
| | | 616.1026 | 3.85 (m, 2H), 2.56 (s, 3H) |
| J164 | | HRMS-ESI (m/z) | ¹H NMR (300 MHz, CDCl₃) δ 8.54 (d, J = 0.5 Hz, 1H), 8.53- |
| | | [M + H]⁺ calcd for | 8.42 (m, 1H), 7.97 (d, J = 8.6 Hz, 1H), 7.88 (dd, J = 12.0, 1.8 |
| | | C₃₁H₂₈F₄N₆O₃S, | Hz, 1H), 7.81-7.75 (m, 2H), 7.53 (d, J = 3.1 Hz, 1H), 7.43- |
| | | 641.1952; found, | 7.34 (m, 4H), 6.92 (s, 1H), 4.00 (d, J = 1.0 Hz, 2H), 2.95 (p, J = |
| | | 641.1956 | 6.9 Hz, 1H), 2.66 (p, J = 6.7 Hz, 1H), 1.29 (dd, J = 6.9, 1.2 Hz, |
| | | | 6H), 1.25-1.14 (m, 6H) |
| J165 | | HRMS-ESI (m/z) | ¹H NMR (300 MHz, CDCl₃) δ 8.57 (s, 1H), 8.43 (d, J = 9.7 Hz, |
| | | [M + H]⁺ calcd for | 1H), 8.37 (s, 1H), 7.85-7.76 (m, 2H), 7.49 (s, 1H), 7.40 (d, J = |
| | | C₃₀H₂₁F₉N₆O₄S, | 8.8 Hz, 3H), 7.34 (d, J = 7.0 Hz, 1H), 7.05 (s, 1H), 4.67-4.45 |
| | | 733.1274; found, | (m, 2H), 3.96 (d, J = 1.2 Hz, 2H), 3.77-3.62 (m, 2H), 2.45 (s, |
| | | 733.127 | 3H) |
| J166 | | HRMS-ESI (m/z) | ¹H NMR (300 MHz, CDCl₃) δ 8.55 (s, 1H), 8.51 (d, J = 8.7 Hz, |
| | | [M + H]⁺ calcd for | 1H), 8.19 (d, J = 1.9 Hz, 1H), 8.08 (dd, J = 8.7, 1.9 Hz, 1H), |
| | | C₃₀H₂₇ClF₃N₇O₃S, | 7.83-7.75 (m, 3H), 7.45-7.30 (m, 3H), 6.89 (dd, J = 8.8, 2.8 |
| | | 658.1609; found, | Hz, 1H), 6.36 (d, J = 2.7 Hz, 1H), 3.99 (s, 2H), 2.97 (s, 6H), |
| | | 658.1617 | 2.59 (p, J = 6.8 Hz, 1H), 1.25-1.08 (m, 6H) |
| J167 | | HRMS-ESI (m/z) | ¹H NMR (400 MHz, CDCl₃) δ 8.63 (s, 1H), 8.20 (d, J = 8.5 Hz, |
| | | [M + H]⁺ calcd for | 1H), 8.05 (d, J = 8.8 Hz, 1H), 8.00 (s, 1H), 7.91 (d, J = 8.5 Hz, |
| | | C₃₀H₂₄F₆N₆O₃S, | 2H), 7.79 (d, J = 8.5 Hz, 2H), 7.43 (d, J = 7.7 Hz, 1H), 7.38- |
| | | 663.1608; found, | 7.31 (m, 1H), 7.06 (s, 2H), 4.63 (d, J = 12.4 Hz, 1H), 4.51 (d, J = |
| | | 663.161 | 12.4 Hz, 1H), 3.94 (d, J = 2.1 Hz, 2H), 3.73 (q, J = 8.7 Hz, |
| | | | 2H), 2.45 (s, 3H), 2.26 (s, 3H); |
| | | | ¹⁹F NMR (376 MHz, CDCl₃) δ −62.47, −73.75 |
| J168 | | | ¹H NMR (400 MHz, CDCl₃) δ 8.63 (s, 1H), 8.49 (t, J = 8.4 Hz, |
| | | | 1H), 8.05-7.95 (m, 1H), 7.95-7.86 (m, 3H), 7.80 (d, J = 8.6 |
| | | | Hz, 2H), 7.54-7.31 (m, 3H), 7.04 (s, 1H), 4.63 (d, J = 12.4 Hz, |
| | | | 1H), 4.50 (d, J = 12.4 Hz, 1H), 4.06-3.86 (m, 2H), 3.72 (qd, J = |
| | | | 8.7, 4.2 Hz, 2H), 2.45 (s, 3H); |
| | | | ¹⁹F NMR (376 MHz, CDCl₃) δ −62.50, −73.78, −131.30 |
| J169 | | | ¹H NMR (400 MHz, CDCl₃) δ 8.54 (s, 1H), 8.49 (t, J = 8.4 Hz, |
| | | | 1H), 8.00-7.94 (m, 1H), 7.89 (dd, J = 12.0, 1.9 Hz, 1H), 7.82- |
| | | | 7.75 (m, 2H), 7.54 (d, J = 3.0 Hz, 1H), 7.43-7.36 (m, 2H), |
| | | | 7.30 (d, J = 8.6 Hz, 1H), 7.01 (dd, J = 8.6, 2.6 Hz, 1H), 6.64 (d, |
| | | | J = 2.6 Hz, 1H), 4.11-4.01 (m, 2H), 3.99 (d, J = 2.8 Hz, 2H), |
| | | | 2.39-2.29 (m, 2H), 1.58 (dt, J = 15.3, 7.4 Hz, 2H), 1.42 (t, J = |
| | | | 7.0 Hz, 3H), 0.92 (t, J = 7.4 Hz, 3H); ¹⁹F NMR (376 MHz, |
| | | | CDCl₃) δ −58.02, −131.01 |
| J170 | | HRMS-ESI (m/z) | ¹H NMR (400 MHz, CDCl₃) δ 8.53 (s, 1H), 8.48 (d, J = 8.4 Hz, |
| | | [M + H]⁺ calcd for | 1H), 7.90-7.86 (m, 3H), 7.81-7.77 (m, 2H), 7.42-7.31 (m, |
| | | C₃₁H₂₉F₃N₆O₅S, | 4H), 6.93 (s, 1H), 5.27 (q, J = 6.6 Hz, 2H), 3.98 (d, J = 1.2 Hz, |
| | | 655.1945; found, | 2H), 3.44 (s, 3H), 2.69 (p, J = 6.8 Hz, 1H), 2.39 (s, 3H), 1.20 |
| | | 655.1953 | (dd, J = 12.6, 6.9 Hz, 6H); ¹⁹F NMR (376 MHz, CDCl₃) δ −58.03 |
| J171 | | HRMS-ESI (m/z) | ¹H NMR (400 MHz, CDCl₃) δ 8.53 (s, 1H), 8.49 (d, J = 8.4 Hz, |
| | | [M + H]⁺ calcd for | 1H), 7.91-7.85 (m, 3H), 7.83-7.77 (m, 2H), 7.38 (d, J = 8.6 |
| | | C₃₀H₂₄F₆N₆O₆S, | Hz, 2H), 7.33-7.28 (m, 1H), 7.10 (d, J = 2.2 Hz, 1H), 7.00 (d, |
| | | 711.1455; found, | J = 8.5 Hz, 1H), 5.30-5.25 (m, 2H), 4.45-4.28 (m, 2H), 4.01- |
| | | 711.1464 | 3.90 (m, 2H), 3.46 (s, 3H), 2.40 (s, 3H); |
| | | | ¹⁹F NMR (376 MHz, CDCl₃) δ −58.02, −74.05 |
| J172 | | HRMS-ESI (m/z) | ¹H NMR (400 MHz, CDCl₃) δ 8.53 (s, 1H), 8.49 (d, J = 8.5 Hz, |
| | | [M + H]⁺ calcd for | 1H), 7.93-7.90 (m, 2H), 7.90-7.85 (m, 1H), 7.83-7.76 (m, |
| | | C₃₂H₃₂F₃N₇O₅S, | 2H), 7.40-7.36 (m, 2H), 7.34 (d, J = 8.8 Hz, 1H), 6.90 (dd, J = |
| | | 684.221; found, | 8.8, 2.8 Hz, 1H), 6.39 (d, J = 2.7 Hz, 1H), 5.31-5.24 (m, 2H), |
| | | 684.2219 | 3.97 (d, J = 0.8 Hz, 2H), 3.45 (s, 3H), 2.97 (s, 6H), 2.65-2.56 |
| | | | (m, 1H), 1.17 (dd, J = 11.9, 6.9 Hz, 6H); |
| | | | ¹⁹F NMR (376 MHz, CDCl₃) δ −58.02 |
| J173 | | HRMS-ESI (m/z) | ¹H NMR (400 MHz, CDCl₃) δ 8.53 (s, 1H), 8.49 (d, J = 8.5 Hz, |
| | | [M + H]⁺ calcd for | 1H), 7.92-7.85 (m, 2H), 7.85-7.77 (m, 3H), 7.44 (d, J = 7.8 |
| | | C₃₁H₂₆F₆N₆O₆S, | Hz, 1H), 7.37 (t, J = 9.4 Hz, 3H), 7.06 (d, J = 1.6 Hz, 1H), 5.26 |
| | | 725.1611; found, | (s, 2H), 4.63 (d, J = 12.5 Hz, 1H), 4.51 (d, J = 12.4 Hz, 1H), |
| | | 725.1618 | 3.95 (d, J = 1.8 Hz, 2H), 3.77-3.69 (m, 2H), 3.45 (s, 3H), 2.45 |
| | | | (s, 3H); ¹⁹F NMR (376 MHz, CDCl₃) δ −58.02, −73.77 |
| J174 | | HRMS-ESI (m/z) | ¹H NMR (400 MHz, CDCl₃) δ 8.53 (s, 1H), 8.48 (dd, J = 8.5, 4.7 |
| | | [M + H]⁺ calcd for | Hz, 1H), 7.92-7.85 (m, 2H), 7.81-7.77 (m, 3H), 7.58 (dd, J = |
| | | C₃₂H₃₁F₃N₆O₆S, | 8.0, 3.8 Hz, 1H), 7.38 (d, J = 8.2 Hz, 3H), 6.94 (s, 1H), 5.26 (d, |
| | | 685.2051; found, | J = 10.2 Hz, 2H), 4.35-4.23 (m, 1H), 3.98 (s, 2H), 3.43 (s, |
| | | 685.2051 | 3H), 3.41-3.15 (m, 2H), 2.42 (s, 3H), 1.38 (dd, J = 23.0, 6.4 |
| | | | Hz, 3H), 1.16-1.09 (m, 3H); |
| | | | ¹⁹F NMR (376 MHz, CDCl₃) δ −58.03 |
| J175 | | HRMS-ESI (m/z) | ¹H NMR (500 MHz, CDCl₃) δ 8.53 (s, 1H), 8.37 (s, 1H), 7.82 (d, |
| | | [M + H]⁺ calcd for | J = 1.8 Hz, 1H), 7.81-7.77 (m, 2H), 7.68 (dd, J = 8.2, 1.9 Hz, |
| | | C₂₉H₂₅F₃N₆O₄S, | 1H), 7.49 (s, 1H), 7.38 (dd, J = 8.3, 5.2 Hz, 3H), 7.33 (d, J = |
| | | 611.1683; found, | 8.3 Hz, 1H), 7.19 (d, J = 8.4 Hz, 1H), 6.91 (s, 1H), 4.00 (d, J = |
| | | 611.1681 | 4.3 Hz, 2H), 2.65 (dd, J = 13.8, 6.8 Hz, 1H), 2.39 (s, 3H), 1.20 |
| | | | (dd, J = 13.0, 6.8 Hz, 6H) |
| J176 | | HRMS-ESI (m/z) | ¹H NMR (400 MHz, CDCl₃) δ 8.54 (s, 1H), 8.33 (s, 1H), 7.82 (d, |
| | | [M + H]⁺ calcd for | J = 1.8 Hz, 1H), 7.81-7.75 (m, 2H), 7.68 (dd, J = 8.3, 1.9 Hz, |
| | | C₂₈H₂₀F₆N₆O₅S, | 1H), 7.45 (s, 1H), 7.38 (d, J = 8.6 Hz, 2H), 7.31 (d, J = 8.0 Hz, |
| | | 667.1193; found, | 1H), 7.22 (d, J = 8.3 Hz, 1H), 7.08 (s, 1H), 7.00 (d, J = 2.0 Hz, |
| | | 667.1199 | 1H), 4.44-4.30 (m, 2H), 4.03-3.91 (m, 2H), 2.40 (s, 3H); ¹⁹F |
| | | | NMR (376 MHz, CDCl₃) δ −58.02, −74.08 |
| J177 | | HRMS-ESI (m/z) | ¹H NMR (400 MHz, CDCl₃) δ 8.54 (s, 1H), 7.84-7.75 (m, 2H), |
| | | [M + H]⁺ calcd for | 7.69-7.62 (m, 2H), 7.42-7.36 (m, 3H), 7.33 (d, J = 8.8 Hz, |
| | | C₃₀H₂₈F₃N₇O₄S, | 1H), 6.88 (d, J = 8.7 Hz, 1H), 6.36 (d, J = 2.8 Hz, 1H), 4.00 (d, |
| | | 640.1948; found, | J = 1.4 Hz, 2H), 2.95 (s, 6H), 2.58 (p, J = 6.9 Hz, 1H), 1.17 (dd, |
| | | 640.1947 | J = 10.0, 6.8 Hz, 6H); ¹⁹F NMR (376 MHz, CDCl₃) δ −58.02 |
| J178 | | HRMS-ESI (m/z) | ¹H NMR (400 MHz, CDCl₃) δ 8.54 (s, 1H), 8.22 (s, 1H), 7.83- |
| | | [M + H]⁺ calcd for | 7.76 (m, 2H), 7.68 (dd, J = 8.4, 1.8 Hz, 1H), 7.39 (ddd, J = |
| | | C₂₉H₂₂F₆N₆O₅S, | 21.6, 14.1, 7.6 Hz, 5H), 7.05 (s, 1H), 4.62 (d, J = 12.4 Hz, 1H), |
| | | 681.1349; found, | 4.50 (d, J = 12.5 Hz, 1H), 3.97 (s, 2H), 3.77-3.68 (m, 2H), |
| | | 681.135 | 2.45 (s, 3H); ¹⁹F NMR (376 MHz, CDCl₃) δ −58.02, −73.74 |
| J179 | | ESIMS m/z | ¹H NMR (400 MHz, CDCl₃) δ 8.54 (s, 1H), 8.06 (s, 1H), 7.83- |
| | | 641 ([M + H]⁺) | 7.75 (m, 3H), 7.69 (dt, J = 8.4, 2.3 Hz, 1H), 7.56 (dd, J = 8.0, |
| | | | 4.5 Hz, 1H), 7.47 (d, J = 16.9 Hz, 1H), 7.41-7.35 (m, 3H), |
| | | | 7.31 (d, J = 8.3 Hz, 1H), 6.92 (s, 1H), 4.26 (dq, J = 12.7, 6.4 |
| | | | Hz, 1H), 4.05-3.94 (m, 2H), 3.42-3.18 (m, 2H), 2.42 (s, 3H), |
| | | | 1.38 (dd, J = 15.6, 6.5 Hz, 3H), 1.14 (td, J = 7.0, 4.4 Hz, 3H); |
| | | | ¹⁹F NMR (376 MHz, CDCl₃) δ −58.02 |
| J180 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.96 (dd, J = 8.6, 1.5 Hz, 1H), 7.88 (dd, J = 12.0, 1.9 Hz, |
| | | C₂₉H₂₄F₄N₆O₄S, | 1H), 7.85-7.72 (m, 2H), 7.51 (d, J = 3.1 Hz, 1H), 7.45-7.29 |
| | | 629.1589; found, | (m, 4H), 7.01 (d, J = 1.7 Hz, 1H), 4.46-4.31 (m, 2H), 3.95 (d, |
| | | 629.1596 | J = 0.8 Hz, 2H), 3.41 (q, J = 7.0 Hz, 2H), 2.43 (s, 3H), 1.15 (t, J = |
| | | | 7.0 Hz, 3H); ¹⁹F NMR (376 MHz, CDCl₃) δ −58.02, −131.14 |
| J181 | | HRMS-ESI (m/z) | ¹H NMR (400 MHz, CDCl₃) δ 8.54 (s, 1H), 8.44 (t, J = 8.3 Hz, |
| | | [M + H]⁺ calcd for | 1H), 7.98 (dd, J = 8.6, 1.4 Hz, 1H), 7.88 (dd, J = 12.0, 1.9 Hz, |
| | | C₂₉H₂₁F₇N₆O₄S, | 1H), 7.84-7.72 (m, 2H), 7.68 (d, J = 8.0 Hz, 1H), 7.48-7.34 |
| | | 683.1306; found, | (m, 4H), 7.09-6.94 (m, 1H), 4.48 (q, J = 6.3 Hz, 1H), 4.10- |
| | | 683.1311 | 3.92 (m, 2H), 3.43 (s, 3H), 2.46 (s, 3H); |
| | | | ¹⁹F NMR (376 MHz, CDCl₃) δ −58.02, −76.42, −131.88 |
| J182 | | HRMS-ESI (m/z) | ¹H NMR (400 MHz, CDCl₃) δ 8.54 (s, 1H), 8.50 (t, J = 8.4 Hz, |
| | | [M + H]⁺ calcd for | 1H), 7.97 (dd, J = 8.8, 1.4 Hz, 1H), 7.88 (dd, J = 12.1, 1.9 Hz, |
| | | C₂₈H₂₀F₇N₇O₃S, | 1H), 7.83-7.72 (m, 2H), 7.62 (d, J = 9.0 Hz, 1H), 7.54 (d, J = |
| | | 668.1309; found, | 3.0 Hz, 1H), 7.43-7.30 (m, 2H), 6.86-6.75 (m, 1H), 6.44 (d, |
| | | 668.1314 | J = 2.6 Hz, 1H), 3.97 (q, J = 18.0 Hz, 2H), 3.08 (s, 6H); ¹⁹F |
| | | | NMR (376 MHz, CDCl₃) δ −58.02, −59.67, −131.38 |
| J183 | | HRMS-ESI (m/z) | ¹H NMR (400 MHz, CDCl₃) δ 8.64 (s, 1H), 8.49 (t, J = 8.3 Hz, |
| | | [M + H]⁺ calcd for | 1H), 8.04-7.86 (m, 4H), 7.80 (d, J = 8.5 Hz, 2H), 7.55 (d, J = |
| | | C₃₀H₂₆F₅N₇O₂S, | 3.0 Hz, 1H), 7.09 (d, J = 14.1 Hz, 1H), 6.53 (d, J = 8.3 Hz, 1H), |
| | | 644.1862; found, | 3.99 (d, J = 1.7 Hz, 2H), 2.95-2.80 (m, 6H), 2.67-2.50 (m, |
| | | 644.1872 | 1H), 1.17 (t, J = 6.7 Hz, 6H); ¹⁹F NMR (376 MHz, CDCl₃) |
| | | | δ −62.50, −119.35, −130.97 |
| J184 | | HRMS-ESI (m/z) | ¹H NMR (400 MHz, CDCl₃) δ 8.63 (s, 1H), 8.51 (t, J = 8.4 Hz, |
| | | [M + H]⁺ calcd for | 1H), 7.98 (dd, J = 8.6, 1.5 Hz, 1H), 7.93-7.85 (m, 3H), 7.80 |
| | | C₃₀H₂₄F₇N₇O₃S, | (d, J = 8.5 Hz, 2H), 7.55 (d, J = 3.1 Hz, 1H), 7.01 (d, J = 9.1 |
| | | 696.1622; found, | Hz, 1H), 6.83 (dd, J = 9.1, 3.1 Hz, 1H), 6.56 (d, J = 3.1 Hz, 1H), |
| | | 696.1633 | 4.25-4.10 (m, 2H), 3.94 (d, J = 2.1 Hz, 2H), 2.95 (s, 6H), 2.63- |
| | | | 2.39 (m, 2H); ¹⁹F NMR (376 MHz, CDCl₃) δ −62.50, −64.58, −131.19 |
| J185 | 208-212 | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (dt, J = 8.6, 1.2 Hz, 1H), 7.88 (dd, J = 12.1, 1.9 Hz, |
| | | C₂₇H₁₈F₇N₆O₄S, | 1H), 7.77 (dd, J = 9.0, 6.8 Hz, 3H), 7.52 (d, J = 3.1 Hz, 1H), |
| | | 655.0993; found, | 7.43-7.34 (m, 2H), 7.17-7.10 (m, 1H), 6.81 (d, J = 2.5 Hz, |
| | | 655.1001 | 1H), 4.07-3.80 (m, 2H), 3.92 (s, 3H); ¹⁹F NMR (376 MHz, |
| | | | CDCl₃) δ −58.02, −60.42, −131.42 |
| J186 | | HRMS-ESI (m/z) | ¹H NMR (400 MHz, CDCl₃) δ 8.61 (s, 1H), 8.49 (t, J = 8.4 Hz, |
| | | [M + H]⁺ calcd for | 1H), 7.98 (d, J = 8.6 Hz, 1H), 7.89 (dd, J = 12.0, 1.9 Hz, 1H), |
| | | C₂₉H₂₁F₇N₆O₃S2, | 7.86-7.78 (m, 4H), 7.49 (d, J = 3.1 Hz, 1H), 7.46-7.31 (m, |
| | | 699.1078; found, | 2H), 7.11-6.97 (m, 1H), 4.63 (d, J = 12.4 Hz, 1H), 4.50 (d, J = |
| | | 699.1084 | 12.4 Hz, 1H), 4.04-3.91 (m, 2H), 3.72 (qd, J = 8.7, 3.9 Hz, |
| | | | 2H), 2.45 (s, 3H); ¹⁹F NMR (376 MHz, CDCl₃) |
| | | | δ −42.63, −73.77, −131.29 |
| J187 | | HRMS-ESI (m/z) | ¹H NMR (400 MHz, CDCl₃) δ 8.61 (s, 1H), 8.47 (td, J = 8.3, 4.8 |
| | | [M + H]⁺ calcd for | Hz, 1H), 7.98 (dd, J = 8.6, 1.5 Hz, 1H), 7.95-7.85 (m, 1H), |
| | | C₂₉H₂₄F₄N₆O₃S2, | 7.83 (s, 4H), 7.53 (td, J = 7.1, 6.2, 3.5 Hz, 1H), 7.48-7.33 (m, |
| | | 645.136; found, | 2H), 6.93 (s, 1H), 4.24-4.08 (m, 1H), 4.07-3.90 (m, 2H), |
| | | 645.1365 | 3.15 (d, J = 22.2 Hz, 3H), 2.43 (s, 3H), 1.37 (dd, J = 16.3, 6.5 |
| | | | Hz, 3H); ¹⁹F NMR (376 MHz, CDCl₃) δ −42.62, −131.47 |
| J188 | | HRMS-ESI (m/z) | ¹H NMR (400 MHz, CDCl₃) δ 8.54 (s, 1H), 8.47 (t, J = 8.4 Hz, |
| | | [M + H]⁺ calcd for | 1H), 7.98 (d, J = 8.5 Hz, 1H), 7.89 (dd, J = 12.0, 1.8 Hz, 1H), |
| | | C₃₁H₂₇F₄N₇O₄S, | 7.82-7.75 (m, 2H), 7.57-7.51 (m, 1H), 7.43-7.34 (m, 4H), |
| | | 670.1854; found, | 6.95 (s, 1H), 4.08-3.94 (m, 2H), 3.32 (s, 3H), 2.72 (q, J = 6.8 |
| | | 670.1857 | Hz, 1H), 1.98 (s, 3H), 1.23 (dd, J = 6.8, 4.6 Hz, 6H); ¹⁹F NMR |
| | | | (376 MHz, CDCl₃) δ −58.02, −131.92 |
| J189 | | HRMS-ESI (m/z) | ¹H NMR (400 MHz, CDCl₃) δ 8.54 (s, 1H), 8.46 (t, J = 8.4 Hz, |
| | | [M + H]⁺ calcd for | 1H), 7.98 (d, J = 8.6 Hz, 1H), 7.89 (dd, J = 11.9, 1.9 Hz, 1H), |
| | | C₃₂H₂₉F₄N₇O₄S, | 7.82-7.76 (m, 2H), 7.54 (d, J = 8.4 Hz, 1H), 7.39 (d, J = 8.6 |
| | | 684.2011; found, | Hz, 3H), 7.35-7.31 (m, 1H), 6.92 (d, J = 2.2 Hz, 1H), 4.09- |
| | | 684.2014 | 3.95 (m, 2H), 3.84-3.71 (m, 1H), 2.74 (p, J = 6.8 Hz, 1H), |
| | | | 1.92 (s, 3H), 1.26-1.22 (m, 6H), 1.17 (t, J = 7.1 Hz, 3H); ¹⁹F |
| | | | NMR (376 MHz, CDCl₃) δ −58.02, −132.07 |
| J190 | 208-212 | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (d, J = 8.5 Hz, 1H), 7.89 (d, J = 12.1 Hz, 1H), 7.77 |
| | | C₂₈H₂₀F₇N₆O₄S, | (dd, J = 14.4, 8.9 Hz, 3H), 7.50 (s, 1H), 7.39 (d, J = 8.5 Hz, |
| | | 669.1149; found, | 2H), 7.11 (d, J = 8.8 Hz, 1H), 6.79 (d, J = 2.5 Hz, 1H), 4.13 (q, |
| | | 669.1148 | J = 7.1 Hz, 2H), 3.98 (q, J = 18.0 Hz, 2H), 1.47 (t, J = 6.9 Hz, |
| | | | 3H); ¹⁹F NMR (376 MHz, CDCl₃) δ −58.02, −60.40, −131.49 |
| J191 | 175-185 | HRMS-ESI (m/z) | ¹H NMR (400 MHz, CDCl₃) δ 8.54 (s, 1H), 8.48 (t, J = 8.3 Hz, |
| | | [M + H]⁺ calcd for | 1H), 7.97 (d, J = 8.6 Hz, 1H), 7.89 (dd, J = 12.0, 1.8 Hz, 1H), |
| | | C₃₀H₂₇F₄N₆O₄S, | 7.82-7.74 (m, 2H), 7.56-7.51 (m, 1H), 7.38 (dd, J = 8.7, 2.4 |
| | | 643.1745; found, | Hz, 3H), 7.06 (dd, J = 8.7, 2.7 Hz, 1H), 6.60 (d, J = 2.7 Hz, 1H), |
| | | 643.1747 | 4.12-3.98 (m, 4H), 2.63 (q, J = 7.2 Hz, 1H), 1.22-1.09 (m, |
| | | | 9H); ¹⁹F NMR (376 MHz, CDCl₃) δ −58.02, −131.02 |
| J192 | | HRMS-ESI (m/z) | ¹H NMR (400 MHz, CDCl₃) δ 8.54 (s, 1H), 8.50 (t, J = 8.4 Hz, |
| | | [M + H]⁺ calcd for | 1H), 8.02-7.92 (m, 1H), 7.88 (dd, J = 12.0, 1.9 Hz, 1H), 7.84- |
| | | C₂₉H₂₅F₄N₇O₄S, | 7.72 (m, 2H), 7.57 (d, J = 3.0 Hz, 1H), 7.43-7.34 (m, 2H), |
| | | 644.1698; found, | 7.00 (d, J = 9.1 Hz, 1H), 6.84 (dd, J = 9.1, 3.1 Hz, 1H), 6.56 (d, |
| | | 644.1705 | J = 3.1 Hz, 1H), 4.07-3.87 (m, 4H), 2.93 (s, 6H), 1.28 (t, J = |
| | | | 7.0 Hz, 3H); ¹⁹F NMR (376 MHz, CDCl₃) δ −58.02, −58.02, −131.05 |
| J193 | | HRMS-ESI (m/z) | ¹H NMR (400 MHz, CDCl₃) δ 8.54 (s, 1H), 8.50 (t, J = 8.4 Hz, |
| | | [M + H]⁺ calcd for | 1H), 7.97 (dd, J = 8.6, 1.5 Hz, 1H), 7.88 (dd, J = 12.1, 1.9 Hz, |
| | | C₂₉H₂₂F₇N₇O₄S, | 1H), 7.83-7.72 (m, 2H), 7.55 (d, J = 3.1 Hz, 1H), 7.43-7.34 |
| | | 698.1415; found, | (m, 2H), 7.04 (d, J = 9.2 Hz, 1H), 6.81 (dd, J = 9.1, 3.1 Hz, 1H), |
| | | 698.1422 | 6.54 (d, J = 3.1 Hz, 1H), 4.28 (qq, J = 11.6, 8.2 Hz, 2H), 4.04- |
| | | | 3.88 (m, 2H), 2.97 (s, 6H); ¹⁹F NMR (376 MHz, CDCl₃) |
| | | | δ −58.02, −58.02, −74.37, −74.37, −131.31 |
| J194 | | HRMS-ESI (m/z) | ¹H NMR (400 MHz, CDCl₃) δ 8.53 (d, J = 9.3 Hz, 2H), 8.19 (d, |
| | | [M + H]⁺ calcd for | J = 1.9 Hz, 1H), 8.14-8.02 (m, 1H), 7.84-7.74 (m, 2H), 7.69 |
| | | C₂₉H₂₁ClF₆N₆O₄S, | (s, 1H), 7.46-7.32 (m, 3H), 7.05 (s, 1H), 4.64 (d, J = 12.5 Hz, |
| | | 699.101; found, | 1H), 4.51 (d, J = 12.5 Hz, 1H), 4.02-3.88 (m, 2H), 3.72 (qd, J = |
| | | 699.1013 | 8.8, 3.8 Hz, 2H), 2.45 (s, 3H); |
| | | | ¹⁹F NMR (376 MHz, CDCl₃) δ −58.02, −73.75 |
| J195 | | HRMS-ESI (m/z) | ¹H NMR (400 MHz, CDCl₃) δ 8.61 (s, 1H), 8.50 (t, J = 8.4 Hz, |
| | | [M + H]⁺ calcd for | 1H), 7.97 (d, J = 8.6 Hz, 1H), 7.89 (dd, J = 11.9, 1.8 Hz, 1H), |
| | | C₂₉H₂₄F₄N₆O₃S2, | 7.83 (s, 3H), 7.51 (d, J = 3.0 Hz, 1H), 7.40 (d, J = 7.8 Hz, 1H), |
| | | 645.136; found, | 7.32 (s, 1H), 7.02 (s, 1H), 4.51-4.27 (m, 2H), 3.95 (s, 2H), |
| | | 645.1357 | 3.56 (s, 1H), 3.41 (q, J = 7.0 Hz, 2H), 2.43 (s, 3H), 1.15 (t, J = |
| | | | 7.0 Hz, 3H); ¹⁹F NMR (376 MHz, CDCl₃) δ −42.63, −131.10 |
| J196 | | HRMS-ESI (m/z) | ¹H NMR (400 MHz, CDCl₃) δ 8.54 (s, 1H), 8.50 (t, J = 8.4 Hz, |
| | | [M + H]⁺ calcd for | 1H), 7.97 (dd, J = 8.6, 1.5 Hz, 1H), 7.88 (dd, J = 12.0, 1.8 Hz, |
| | | C₃₀H₂₇F₄N₇O₃S, | 1H), 7.83-7.73 (m, 2H), 7.58 (d, J = 3.0 Hz, 1H), 7.43-7.34 |
| | | 642.1905; found, | (m, 2H), 7.24 (s, 1H), 6.83 (dd, J = 8.7, 2.7 Hz, 1H), 6.40 (d, J = |
| | | 642.191 | 2.7 Hz, 1H), 3.98 (d, J = 1.9 Hz, 2H), 2.97 (s, 6H), 2.36- |
| | | | 2.23 (m, 2H), 0.91 (t, J = 7.4 Hz, 3H); |
| | | | ¹⁹F NMR (376 MHz, CDCl₃) δ −58.02, −58.02, −130.77 |
| J197 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (d, J = 8.6 Hz, 1H), 7.89 (dd, J = 12.0, 1.9 Hz, 1H), |
| | | C₂₈H₂₂F₄N₆O₄S, | 7.83-7.73 (m, 2H), 7.55 (s, 1H), 7.37 (dd, J = 12.3, 8.6 Hz, |
| | | 615.1432; found, | 2H), 7.00 (dd, J = 8.7, 2.7 Hz, 1H), 6.58 (d, J = 2.7 Hz, 1H), |
| | | 615.1431 | 4.97 (s, 1H), 3.99 (d, J = 2.8 Hz, 2H), 2.62 (p, J = 7.0 Hz, 2H), |
| | | | 1.18 (dd, J = 6.9, 4.4 Hz, 6H); ¹⁹F NMR (376 MHz, CDCl₃) |
| | | | δ −58.02, −130.99 |
| J198 | | HRMS-ESI (m/z) | ¹H NMR (400 MHz, CDCl₃) δ 8.65-8.57 (m, 1H), 8.56-8.45 |
| | | [M + H]⁺ calcd for | (m, 2H), 7.97 (d, J = 8.5 Hz, 1H), 7.88 (dd, J = 12.0, 1.8 Hz, |
| | | C₃₀H₂₅F₄N₇O₃S, | 1H), 7.83-7.75 (m, 2H), 7.55-7.43 (m, 2H), 7.43-7.34 (m, |
| | | 640.1748; found, | 2H), 7.32-7.28 (m, 1H), 7.25-7.17 (m, 2H), 3.97 (d, J = 2.4 |
| | | 640.1757 | Hz, 2H), 2.79 (tq, J = 11.2, 5.9, 4.7 Hz, 3H), 1.59 (s, 6H); ¹⁹F |
| | | | NMR (376 MHz, CDCl₃) δ −58.02, −131.18 |
| J199 | | HRMS-ESI (m/z) | ¹H NMR (400 MHz, CDCl₃) δ 8.54 (s, 1H), 8.19 (d, J = 8.5 Hz, |
| | | [M + H]⁺ calcd for | 1H), 8.04 (d, J = 8.6 Hz, 1H), 7.99 (s, 1H), 7.82-7.77 (m, 2H), |
| | | C₃₂H₃₀F₃N₇O₄S, | 7.53 (d, J = 8.6 Hz, 1H), 7.38 (d, J = 8.7 Hz, 2H), 7.34 (d, J = |
| | | 666.2105; found, | 7.8 Hz, 1H), 6.99 (d, J = 8.5 Hz, 2H), 4.07-3.92 (m, 2H), 3.31 |
| | | 666.2107 | (s, 3H), 2.81-2.70 (m, 1H), 2.23 (s, 3H), 1.98 (s, 3H), 1.28- |
| | | | 1.20 (m, 6H); |
| | | | ¹⁹F NMR (376 MHz, CDCl₃) δ −58.02 |
| J200 | | HRMS-ES (m/z) | ¹H NMR (400 MHz, CDCl₃) δ 8.54 (s, 1H), 8.20 (d, J = 8.5 Hz, |
| | | [M + H]⁺ calcd for | 1H), 8.05 (d, J = 8.5 Hz, 1H), 7.99 (s, 1H), 7.82-7.76 (m, 2H), |
| | | C₃₃H₃₂F₃N₇O₄S, | 7.56-7.52 (m, 1H), 7.38 (d, J = 8.6 Hz, 2H), 7.31 (d, J = 8.3 |
| | | 680.2261; found, | Hz, 1H), 6.96 (d, J = 15.5 Hz, 2H), 4.08-3.91 (m, 2H), 3.78 |
| | | 680.2265 | (dt, J = 14.9, 6.8 Hz, 2H), 2.82-2.71 (m, 1H), 2.22 (s, 3H), |
| | | | 1.92 (s, 3H), 1.25 (d, J = 6.8 Hz, 6H), 1.16 (t, J = 7.1 Hz, 3H); |
| | | | ¹⁹F NMR (376 MHz, CDCl₃) δ −58.02 |
| J201 | 232-238 | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (d, J = 8.3 Hz, 1H), 7.89 (dd, J = 12.0, 1.9 Hz, 1H), |
| | | C₂₈H₂₀F₇N₆O₅S, | 7.84-7.73 (m, 2H), 7.51 (s, 1H), 7.39 (d, J = 8.6 Hz, 2H), 7.07- |
| | | 685.1099; found, | 7.02 (m, 2H), 6.83-6.78 (m, 1H), 4.41-4.22 (m, 2H), 4.09- |
| | | 685.1095 | 3.87 (m, 2H), 3.84 (s, 3H); ¹⁹F NMR (376 MHz, CDCl₃) |
| | | | δ −58.03, −74.28, −131.47 |
| J202 | 232-236 | HRMS-ESI (m/z) | ¹H NMR (400 MHz, CDCl₃) δ 8.54 (s, 1H), 8.47 (t, J = 8.3 Hz, |
| | | [M + H]⁺ calcd for | 1H), 7.98 (d, J = 8.4 Hz, 1H), 7.90 (d, J = 12.2 Hz, 1H), 7.79 (d, |
| | | C₂₉H₂₄F₅N₆O₄S, | J = 9.0 Hz, 2H), 7.53 (d, J = 6.7 Hz, 1H), 7.39 (d, J = 8.6 Hz, |
| | | 647.1494; found, | 2H), 7.17 (d, J = 12.3 Hz, 1H), 6.66 (d, J = 7.9 Hz, 1H), 4.00 (d, |
| | | 647.1526 | J = 3.1 Hz, 2H), 3.89 (s, 3H), 2.60 (m, 1H), 1.20-1.11 (m, 6H); |
| | | | ¹⁹F NMR (376 MHz, CDCl₃) δ −58.02, −131.07, −131.53 |
| J203 | | HRMS-ESI (m/z) | ¹H NMR (400 MHz, CDCl₃) δ 8.54 (s, 1H), 8.47 (td, J = 8.3, 4.9 |
| | | [M + H]⁺ calcd for | Hz, 1H), 7.97 (dd, J = 8.6, 1.6 Hz, 1H), 7.88 (ddd, J = 12.0, 3.2, |
| | | C₂₉H₂₅F₄N₆O₄S, | 1.9 Hz, 1H), 7.83-7.72 (m, 2H), 7.55-7.42 (m, 2H), 7.42- |
| | | 629.1589; found, | 7.35 (m, 2H), 7.27 (d, J = 7.9 Hz, 1H), 7.00 (dd, J = 8.0, 1.3 Hz, |
| | | 629.1595 | 1H), 4.15 (dq, J = 8.3, 6.4 Hz, 1H), 3.99 (dd, J = 3.0, 1.5 Hz, |
| | | | 2H), 3.17 (d, J = 19.7 Hz, 3H), 2.51-2.38 (m, 3H), 1.38 (dd, J = |
| | | | 14.2, 6.4 Hz, 3H); |
| | | | ¹⁹F NMR (376 MHz, CDCl₃) δ −58.02, −131.16, −131.53 |
| J204 | | HRMS-ESI (m/z) | ¹H NMR (400 MHz, CDCl₃) δ 8.61 (s, 1H), 8.19 (d, J = 8.5 Hz, |
| | | [M + H]⁺ calcd for | 1H), 8.04 (d, J = 8.9 Hz, 1H), 7.99 (s, 1H), 7.88-7.76 (m, 3H), |
| | | C₃₀H₂₄F₆N₆O₃S2, | 7.39 (dd, J = 34.8, 7.6 Hz, 2H), 7.04 (d, J = 19.8 Hz, 2H), 4.63 |
| | | 695.1328; found, | (d, J = 12.4 Hz, 1H), 4.51 (d, J = 12.4 Hz, 1H), 4.07-3.86 (m, |
| | | 695.133 | 3H), 3.80-3.63 (m, 2H), 2.43 (d, J = 12.8 Hz, 3H), 2.32-2.18 |
| | | | (m, 3H); ¹⁹F NMR (376 MHz, CDCl₃) δ −42.67, −73.75 |
| J205 | | HRMS-ESI (m/z) | ¹H NMR (400 MHz, CDCl₃) δ 8.61 (s, 1H), 8.21 (d, J = 8.5 Hz, |
| | | [M + H]⁺ calcd for | 1H), 8.09-7.94 (m, 2H), 7.91-7.73 (m, 3H), 7.42 (d, J = 7.8 |
| | | C₃₀H₂₇F₃N₆O₃S2, | Hz, 1H), 7.30 (d, J = 8.2 Hz, 2H), 7.06 (d, J = 23.7 Hz, 2H), |
| | | 641.1611; found, | 4.52-4.28 (m, 3H), 3.94 (s, 2H), 3.43 (q, J = 7.0 Hz, 2H), 2.43 |
| | | 641.161 | (s, 3H), 2.27 (s, 3H), 1.17 (t, J = 7.0 Hz, 3H); |
| | | | ¹⁹F NMR (376 MHz, CDCl₃) δ −42.67 |
| J206 | | HRMS-ESI (m/z) | ¹H NMR (400 MHz, CDCl₃) δ 8.54 (s, 1H), 8.20 (d, J = 8.5 Hz, |
| | | [M + H]⁺ calcd for | 1H), 8.08-7.92 (m, 2H), 7.87-7.72 (m, 2H), 7.40 (dd, J = |
| | | C₃₀H₂₇F₃N₆O₄S, | 13.2, 8.1 Hz, 2H), 7.30 (d, J = 7.4 Hz, 2H), 7.11-6.96 (m, 2H), |
| | | 625.1839; found, | 4.38 (p, J = 14.1, 13.3 Hz, 2H), 3.98 (d, J = 36.9 Hz, 2H), 3.40 |
| | | 625.1843 | (dq, J = 26.3, 7.0 Hz, 2H), 2.41 (d, J = 12.7 Hz, 3H), 2.27 (s, |
| | | | 3H), 1.21-1.11 (m, 3H); |
| | | | ¹⁹F NMR (376 MHz, CDCl₃) δ −58.02 |
| J211 | | HRMS-ESI (m/z) | ¹H NMR (400 MHz, CDCl₃) δ 8.56 (s, 1H), 8.35 (d, J = 8.7 Hz, |
| | | [M + H]⁺ calcd for | 1H), 8.29 (d, J = 8.2 Hz, 1H), 8.21 (s, 1H), 7.83-7.76 (m, 2H), |
| | | C₃₀H₂₅F₅N₆O₃S, | 7.66 (s, 1H), 7.38 (dd, J = 8.3, 5.9 Hz, 3H), 7.34-7.29 (m, |
| | | 645.1702; found, | 1H), 6.90 (s, 1H), 6.67 (t, J = 54.7 Hz, 1H), 3.99 (s, 2H), 2.70- |
| | | 645.1703 | 2.61 (m, 1H), 2.38 (s, 3H), 1.20 (dd, J = 20.9, 6.8 Hz, 6H); ¹⁹F |
| | | | NMR (376 MHz, CDCl₃) δ −58.02, −110.68 (d, J = 19.3 Hz) |
| J212 | | ESIMS m/z | ¹H NMR (400 MHz, CDCl₃) δ 8.56 (s, 1H), 8.37 (d, J = 8.7 Hz, |
| | | 701 ([M + H]⁺) | 1H), 8.29 (d, J = 8.6 Hz, 1H), 8.21 (s, 1H), 7.84-7.74 (m, 2H), |
| | | | 7.62 (s, 1H), 7.40 (d, J = 8.5 Hz, 2H), 7.30 (d, J = 9.0 Hz, 1H), |
| | | | 7.07 (s, 1H), 6.99 (d, J = 8.3 Hz, 1H), 6.69 (t, J = 54.8 Hz, 1H), |
| | | | 4.41-4.31 (m, 2H), 4.03-3.86 (m, 2H), 2.40 (s, 3H); ¹⁹F NMR |
| | | | (376 MHz, CDCl₃) δ −58.02, −74.18, −110.86 (d, J = 23.0 Hz). |
| J213 | | HRMS-ESI (m/z) | ¹H NMR (400 MHz, CDCl₃) δ 8.56 (s, 1H), 8.33 (d, J = 8.7 Hz, |
| | | [M + H]⁺ calcd for | 1H), 8.29 (d, J = 8.9 Hz, 1H), 8.22 (s, 1H), 7.83-7.77 (m, 2H), |
| | | C₃₁H₂₈F₅N₇O₃S, | 7.67 (s, 1H), 7.43-7.37 (m, 2H), 7.32 (d, J = 8.8 Hz, 1H), 6.88 |
| | | 674.1967; found, | (dd, J = 8.8, 2.8 Hz, 1H), 6.68 (t, J = 54.8 Hz, 1H), 6.35 (d, J = |
| | | 674.1979 | 2.7 Hz, 1H), 3.99 (s, 2H), 2.96 (s, 6H), 2.59 (p, J = 6.7 Hz, 1H), |
| | | | 1.18 (dd, J = 18.7, 6.8 Hz, 6H); |
| | | | ¹⁹F NMR (376 MHz, CDCl₃) δ −58.02, −110.81 |
| J214 | | HRMS-ESI (m/z) | ¹H NMR (400 MHz, CDCl₃) δ 8.56 (s, 1H), 8.35 (d, J = 8.6 Hz, |
| | | [M + H]⁺ calcd for | 1H), 8.29 (d, J = 8.9 Hz, 1H), 8.22 (s, 1H), 7.83-7.76 (m, 2H), |
| | | C₃₀H₂₅F₅N₆O₄S, | 7.62 (s, 1H), 7.39 (d, J = 8.3 Hz, 3H), 7.29 (d, J = 7.9 Hz, 1H), |
| | | 661.1651; found, | 7.02 (s, 1H), 6.69 (t, J = 54.8 Hz, 1H), 4.47-4.34 (m, 2H), |
| | | 661.1659 | 3.95 (s, 2H), 3.40 (q, J = 7.0 Hz, 2H), 2.43 (s, 3H), 1.15 (t, J = |
| | | | 7.0 Hz, 3H); ¹⁹F NMR (376 MHz, CDCl₃) δ −58.02, −110.86 (d, J = |
| | | | 32.2 Hz) |
| J215 | | HRMS-ESI (m/z) | ¹H NMR (400 MHz, CDCl₃) δ 8.56 (s, 1H), 8.35 (d, J = 8.6 Hz, |
| | | [M + H]⁺ calcd for | 1H), 8.29 (d, J = 8.7 Hz, 1H), 8.21 (s, 1H), 7.83-7.77 (m, 2H), |
| | | C₃₀H₂₂F₈N₆O₄S, | 7.60 (s, 1H), 7.40 (t, J = 6.8 Hz, 3H), 7.34 (d, J = 7.8 Hz, 1H), |
| | | 715.1368; found, | 7.05 (s, 1H), 6.68 (t, J = 54.8 Hz, 1H), 4.68-4.46 (m, 2H), |
| | | 715.1377 | 3.96 (d, J = 2.2 Hz, 2H), 3.71 (qd, J = 8.7, 3.0 Hz, 2H), 2.45 (s, |
| | | | 3H); ¹⁹F NMR (376 MHz, CDCl₃) δ −58.02, −73.79, −110.72 (d, J = |
| | | | 66.1 Hz) |
| J216 | 202-214 | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (d, J = 8.6 Hz, 1H), 7.89 (dd, J = 12.0, 1.8 Hz, 1H), |
| | | C₂₈H₂₃F₄N₆O₄S, | 7.82-7.75 (m, 2H), 7.56 (s, 1H), 7.39 (d, J = 8.6 Hz, 2H), 7.34 |
| | | 615.1432; found, | (d, J = 8.6 Hz, 1H), 7.04 (dd, J = 8.7, 2.7 Hz, 1H), 6.66 (d, J = |
| | | 615.1433 | 2.6 Hz, 1H), 3.99 (d, J = 1.7 Hz, 2H), 3.83 (s, 3H), 2.41 (q, J = |
| | | | 7.5 Hz, 2H), 1.18 (t, J = 7.6 Hz, 3H); |
| | | | ¹⁹F NMR (376 MHz, CDCl₃) δ −58.02, −130.97 |
| J217 | | HRMS-ESI (m/z) | ¹H NMR (400 MHz, CDCl₃) δ 8.54 (s, 1H), 8.47 (d, J = 8.7 Hz, |
| | | [M + H]⁺ calcd for | 1H), 8.36 (d, J = 1.9 Hz, 1H), 8.12 (d, J = 8.9 Hz, 1H), 7.81- |
| | | C₂₉H₂₄BrF₃N₆O₃S, | 7.76 (m, 2H), 7.72 (s, 1H), 7.39 (dd, J = 8.3, 2.5 Hz, 3H), 7.32 |
| | | 673.0839; found, | (d, J = 8.1 Hz, 1H), 6.90 (s, 1H), 4.00 (s, 2H), 2.67 (dt, J = |
| | | 673.0838 | 13.8, 7.0 Hz, 1H), 2.39 (s, 3H), 1.24 (d, J = 6.8 Hz, 3H), 1.18 |
| | | | (d, J = 6.9 Hz, 3H); ¹⁹F NMR (376 MHz, CDCl₃) δ −58.02 |
| J218 | | HRMS-ESI (m/z) | ¹H NMR (400 MHz, CDCl₃) δ 8.54 (s, 1H), 8.51 (d, J = 8.8 Hz, |
| | | [M + H]⁺ calcd for | 1H), 8.36 (d, J = 1.9 Hz, 1H), 8.12 (d, J = 8.7 Hz, 1H), 7.82- |
| | | C28Hi9BrF₆N₆O₄S, | 7.76 (m, 2H), 7.72 (s, 1H), 7.39 (d, J = 8.6 Hz, 3H), 7.30 (d, J = |
| | | 729.0349; found, | 8.6 Hz, 1H), 7.08 (d, J = 2.1 Hz, 1H), 6.99 (d, J = 8.5 Hz, 1H), |
| | | 729.0345 | 4.43-4.31 (m, 1H), 4.02-3.91 (m, 2H), 2.40 (s, 3H); ¹⁹F NMR |
| | | | (376 MHz, CDCl₃) δ −58.02, −74.06 |
| J219 | | HRMS-ESI (m/z) | ¹H NMR (400 MHz, CDCl₃) δ 8.54 (s, 1H), 8.47 (d, J = 8.7 Hz, |
| | | [M + H]⁺ calcd for | 1H), 8.36 (d, J = 1.9 Hz, 1H), 8.12 (d, J = 8.6 Hz, 1H), 7.83- |
| | | Cao Hz/BrFaN/OaS, | 7.77 (m, 2H), 7.75 (s, 1H), 7.41-7.37 (m, 2H), 7.33 (d, J = 8.8 |
| | | 702.1104; found, | Hz, 1H), 6.89 (dd, J = 8.8, 2.8 Hz, 1H), 6.36 (d, J = 2.8 Hz, 1H), |
| | | 702.1109 | 3.99 (s, 2H), 2.96 (s, 6H), 2.61 (dd, J = 14.7, 7.8 Hz, 1H), 0.90- |
| | | | 0.86 (m, 6H); ¹⁹F NMR (376 MHz, CDCl₃) δ −58.02 |
| J220 | | HRMS-ESI (m/z) | ¹H NMR (400 MHz, CDCl₃) δ 8.54 (d, J = 0.8 Hz, 1H), 8.48 (dd, |
| | | [M + H]⁺ calcd for | J = 8.8, 5.5 Hz, 1H), 8.37-8.33 (m, 1H), 8.13 (d, J = 9.2 Hz, |
| | | C₂₉H₂₄BrF₃N₆O₄S, | 1H), 7.79 (d, J = 8.7 Hz, 2H), 7.68 (d, J = 16.8 Hz, 1H), 7.54 (d, |
| | | 689.0788; found, | J = 8.0 Hz, 1H), 7.39 (d, J = 8.6 Hz, 3H), 6.93 (d, J = 4.1 Hz, |
| | | 689.079 | 1H), 4.16 (dt, J = 19.8, 6.4 Hz, 1H), 4.00 (d, J = 1.6 Hz, 2H), |
| | | | 3.17 (d, J = 46.8 Hz, 3H), 2.43 (s, 3H), 1.39 (dd, J = 37.0, 6.3 |
| | | | Hz, 3H); ¹⁹F NMR (376 MHz, CDCl₃) δ −58.02 |
| J221 | | HRMS-ESI (m/z) | ¹H NMR (400 MHz, CDCl₃) δ 8.54 (s, 1H), 8.50 (d, J = 8.7 Hz, |
| | | [M + H]⁺ calcd for | 1H), 8.36 (d, J = 1.9 Hz, 1H), 8.12 (d, J = 8.6 Hz, 1H), 7.81- |
| | | C₂₉H₂₄BrF₃N₆O₄S, | 7.76 (m, 2H), 7.70 (s, 1H), 7.43-7.37 (m, 3H), 7.30 (d, J = 8.0 |
| | | 689.0788; found, | Hz, 1H), 7.03 (s, 1H), 4.47-4.35 (m, 2H), 3.95 (s, 2H), 3.41 |
| | | 689.0793 | (q, J = 7.0 Hz, 2H), 2.43 (s, 3H), 1.15 (t, J = 7.0 Hz, 3H); ¹⁹F |
| | | | NMR (376 MHz, CDCl₃) δ −58.02 |
| J222 | | HRMS-ESI (m/z) | ¹H NMR (400 MHz, CDCl₃) δ 8.54 (s, 1H), 8.50 (d, J = 8.7 Hz, |
| | | [M + H]⁺ calcd for | 1H), 8.36 (d, J = 1.9 Hz, 1H), 8.13 (dd, J = 8.7, 1.9 Hz, 1H), |
| | | C₂₉H₂iBrF₆N₆O₄S, | 7.81-7.76 (m, 2H), 7.68 (s, 1H), 7.44-7.32 (m, 4H), 7.05 (d, |
| | | 743.0505; found, | J = 1.7 Hz, 1H), 4.64 (d, J = 12.5 Hz, 1H), 4.51 (d, J = 12.5 Hz, |
| | | 743.0507 | 1H), 3.96 (d, J = 2.4 Hz, 2H), 3.72 (qd, J = 8.7, 3.2 Hz, 2H), |
| | | | 2.45 (s, 3H); ¹⁹F NMR (376 MHz, CDCl₃) δ −58.04, −73.73 |
| J223 | | HRMS-ESI (m/z) | ¹H NMR (400 MHz, CDCl₃) δ 8.61 (s, 1H), 8.50 (t, J = 8.4 Hz, |
| | | [M + H]⁺ calcd for | 1H), 7.98 (d, J = 8.5 Hz, 1H), 7.89 (dd, J = 12.1, 1.8 Hz, 1H), |
| | | C₂₈H₁₉F₇N₆O₃S2, | 7.83 (d, J = 1.1 Hz, 4H), 7.51 (d, J = 3.0 Hz, 1H), 7.35-7.28 |
| | | 685.0921; found, | (m, 1H), 7.11-7.04 (m, 1H), 6.99 (d, J = 8.5 Hz, 1H), 4.47- |
| | | 685.0924 | 4.26 (m, 2H), 4.04-3.88 (m, 2H), 2.40 (s, 3H); ¹⁹F NMR (376 MHz, |
| | | | CDCl₃) δ −42.63 (d, J = 1.2 Hz), −74.15, −131.51 |
| J224 | | HRMS-ESI (m/z) | ¹H NMR (400 MHz, CDCl₃) δ 8.61 (s, 1H), 8.51 (t, J = 8.4 Hz, |
| | | [M + H]⁺ calcd for | 1H), 8.01-7.94 (m, 1H), 7.89 (dd, J = 12.0, 1.9 Hz, 1H), 7.83 |
| | | C₂₈H₂₂F₄N₆O₃S2, | (d, J = 1.2 Hz, 4H), 7.55 (d, J = 3.0 Hz, 1H), 7.24 (d, J = 2.1 |
| | | 631.1204; found, | Hz, 1H), 7.02-6.90 (m, 2H), 4.06 (q, J = 7.0 Hz, 2H), 4.02- |
| | | 631.1207 | 3.85 (m, 2H), 2.37 (s, 3H), 1.31 (t, J = 7.0 Hz, 3H); ¹⁹F NMR |
| | | | (376 MHz, CDCl₃) δ −42.63, −42.63, −131.21 |
| J225 | | HRMS-ESI (m/z) | ¹H NMR (400 MHz, CDCl₃) δ 8.61 (s, 1H), 8.49 (t, J = 8.4 Hz, |
| | | [M + H]⁺ calcd for | 1H), 8.02-7.94 (m, 1H), 7.93-7.79 (m, 5H), 7.53 (d, J = 3.0 |
| | | C₃₀H₂₃F₇N₆O₃S2, | Hz, 1H), 7.28 (d, J = 1.9 Hz, 1H), 7.06-7.00 (m, 1H), 6.95 (d, |
| | | 713.1234; found, | J = 8.5 Hz, 1H), 4.14-4.00 (m, 2H), 3.95 (s, 2H), 2.38 (s, 3H), |
| | | 713.1237 | 2.26-2.08 (m, 2H), 2.06-1.87 (m, 2H); ¹⁹F NMR (376 MHz, |
| | | | CDCl₃) δ −42.63, −66.31, −66.32, −66.36, −131.35 |
| J226 | | | ¹H NMR (400 MHz, CDCl₃) δ 8.62 (s, 1H), 8.54 (d, J = 8.7 Hz, |
| | | | 1H), 8.19 (d, J = 1.9 Hz, 1H), 8.09 (dd, J = 8.7, 1.9 Hz, 1H), |
| | | | 7.83 (d, J = 1.3 Hz, 4H), 7.69 (s, 1H), 7.42 (d, J = 7.8 Hz, 1H), |
| | | | 7.38-7.29 (m, 1H), 7.05 (s, 1H), 4.64 (d, J = 12.4 Hz, 1H), |
| | | | 4.51 (d, J = 12.5 Hz, 1H), 3.96 (d, J = 2.4 Hz, 2H), 3.72 (qd, J = |
| | | | 8.7, 3.7 Hz, 2H), 2.45 (s, 3H); ¹⁹F NMR (376 MHz, CDCl₃) |
| | | | δ −42.62, −73.75 |
| J227 | | HRMS-ESI (m/z) | ¹H NMR (400 MHz, CDCl₃) δ 8.57 (d, J = 4.8 Hz, 2H), 8.37 (d, |
| | | [M + H]⁺ calcd for | J = 9.0 Hz, 2H), 7.78 (dd, J = 10.6, 3.8 Hz, 3H), 7.39 (dd, J = |
| | | C₃₀H₂₄F₃N₇O₃S, | 8.3, 5.5 Hz, 3H), 7.32 (d, J = 8.1 Hz, 1H), 6.88 (s, 1H), 4.01 (d, |
| | | 620.1686; found, | J = 1.4 Hz, 2H), 2.64 (p, J = 6.8 Hz, 1H), 2.39 (s, 3H), 1.21 (dd, |
| | | 620.1687 | J = 22.3, 6.9 Hz, 6H); |
| | | | ¹⁹F NMR (376 MHz, CDCl₃) δ −58.01 |
| J228 | | HRMS-ESI (m/z) | ¹H NMR (400 MHz, CDCl₃) δ 8.62-8.54 (m, 2H), 8.38 (d, J = |
| | | [M + H]⁺ calcd for | 7.0 Hz, 2H), 7.79 (d, J = 8.6 Hz, 2H), 7.71 (d, J = 21.6 Hz, 1H), |
| | | C₃₀H₂₄F₃N₇O₄S, | 7.53 (d, J = 7.7 Hz, 1H), 7.40 (d, J = 8.3 Hz, 3H), 6.91 (s, 1H), |
| | | 636.1635; found, | 4.15 (dd, J = 14.5, 6.5 Hz, 1H), 4.01 (d, J = 1.3 Hz, 2H), 3.17 |
| | | 636.1636 | (d, J = 40.9 Hz, 3H), 2.45-2.41 (m, 3H), 1.38 (dd, J = 30.8, |
| | | | 6.5 Hz, 3H);¹⁹F NMR (376 MHz, CDCl₃) δ −58.01 |
| J229 | | | ¹H NMR (400 MHz, CDCl₃) δ 8.62 (s, 1H), 8.45 (t, J = 8.3 Hz, |
| | | | 1H), 7.99 (d, J = 8.7 Hz, 1H), 7.89 (dd, J = 12.0, 1.8 Hz, 1H), |
| | | | 7.83 (s, 4H), 7.71-7.63 (m, 1H), 7.44 (d, J = 8.1 Hz, 1H), 7.38 |
| | | | (d, J = 3.1 Hz, 1H), 7.02 (s, 1H), 4.00 (d, J = 8.8 Hz, 2H), 3.43 |
| | | | (s, 3H), 3.39-3.32 (m, 1H), 2.47 (s, 3H); |
| | | | ¹⁹F NMR (376 MHz, CDCl₃) δ −42.62 (d, J = 3.4 |
| | | | Hz), −75.56, −76.42, −76.62, −131.00, −131.84 |
| J230 | | | ¹H NMR (400 MHz, CDCl₃) δ 8.61 (s, 1H), 8.51-8.43 (m, 1H), |
| | | | 7.98 (d, J = 8.6 Hz, 1H), 7.89 (d, J = 12.1 Hz, 1H), 7.83 (s, 4H), |
| | | | 7.59-7.47 (m, 1H), 7.38 (td, J = 18.0, 16.1, 5.6 Hz, 2H), 6.92 |
| | | | (s, 1H), 4.26 (dq, J = 13.6, 6.5 Hz, 1H), 3.99 (d, J = 1.4 Hz, |
| | | | 2H), 3.45-3.13 (m, 2H), 2.40 (d, J = 18.8 Hz, 3H), 1.35 (dd, J = |
| | | | 6.5, 4.0 Hz, 3H), 1.19-1.08 (m, 3H); ¹⁹F NMR (376 MHz, |
| | | | CDCl₃) δ −42.63, −43.18, −132 |
| J231 | | | ¹H NMR (400 MHz, CDCl₃) δ 8.62 (s, 1H), 8.52 (d, J = 8.7 Hz, |
| | | | 1H), 8.19 (d, J = 1.9 Hz, 1H), 8.09 (d, J = 8.7 Hz, 1H), 7.83 (d, |
| | | | J = 1.3 Hz, 3H), 7.73 (s, 1H), 7.42-7.30 (m, 3H), 6.90 (s, 1H), |
| | | | 4.00 (s, 2H), 2.67 (p, J = 6.8 Hz, 1H), 2.39 (s, 3H), 1.29-1.10 |
| | | | (m, 6H); ¹⁹F NMR (376 MHz, CDCl₃) δ −42.62, −89.42, −161.98 |
| J232 | | | ¹H NMR (400 MHz, CDCl₃) δ 8.62 (s, 1H), 8.52 (d, J = 8.7 Hz, |
| | | | 1H), 8.22-8.16 (m, 1H), 8.10 (d, J = 8.7 Hz, 1H), 7.83 (s, 3H), |
| | | | 7.69 (d, J = 20.5 Hz, 1H), 7.54 (d, J = 8.0 Hz, 1H), 7.39 (d, J = |
| | | | 8.1 Hz, 2H), 6.93 (s, 1H), 4.24-4.06 (m, 1H), 4.00 (d, J = 1.4 |
| | | | Hz, 2H), 3.22 (s, 1H), 3.16-3.08 (m, 2H), 2.48-2.32 (m, 3H), |
| | | | 1.47-1.30 (m, 3H); |
| | | | ¹⁹F NMR (376 MHz, CDCl₃) δ −42.62 |
| J233 | | | ¹H NMR (400 MHz, CDCl₃) δ 8.61 (d, J = 0.7 Hz, 1H), 8.18 (dd, |
| | | | J = 8.5, 3.2 Hz, 1H), 8.04 (d, J = 9.1 Hz, 1H), 7.99 (s, 1H), 7.87- |
| | | | 7.78 (m, 4H), 7.54 (dd, J = 8.0, 3.1 Hz, 1H), 7.38 (t, J = 6.3 |
| | | | Hz, 1H), 7.03 (d, J = 22.3 Hz, 1H), 6.94 (s, 1H), 4.17 (dq, J = |
| | | | 17.7, 6.3 Hz, 1H), 4.02-3.93 (m, 2H), 3.16 (d, J = 38.3 Hz, |
| | | | 3H), 2.42 (d, J = 2.0 Hz, 3H), 2.22 (d, J = 23.7 Hz, 3H), 1.38 |
| | | | (dd, J = 26.1, 6.4 Hz, 3H); ¹⁹F NMR (376 MHz, CDCl₃) |
| | | | δ −23.10, −42.67, −43.22, −88.61, −185.13, −213.97 |
| J234 | | HRMS-ESI (m/z) | ¹H NMR (400 MHz, CDCl₃) δ 8.63 (s, 1H), 8.54 (d, J = 8.6 Hz, |
| | | [M + H]⁺ calcd for | 1H), 8.20 (d, J = 1.9 Hz, 1H), 8.10 (d, J = 8.6 Hz, 1H), 7.90 (d, |
| | | C₂₉H₂₁ClF₆N₆O₃S, | J = 8.4 Hz, 2H), 7.80 (d, J = 8.6 Hz, 2H), 7.69 (s, 1H), 7.46- |
| | | 683.1061; found, | 7.32 (m, 2H), 7.05 (s, 1H), 4.64 (d, J = 12.5 Hz, 1H), 4.51 (d, J = |
| | | 683.1053 | 12.4 Hz, 1H), 3.96 (d, J = 2.4 Hz, 2H), 3.72 (qd, J = 8.8, 3.7 |
| | | | Hz, 2H), 2.45 (s, 3H); |
| | | | ¹⁹F NMR (376 MHz, CDCl₃) δ −62.51, −73.75 |
| J235 | | HRMS-ESI (m/z) | ¹H NMR (400 MHz, CDCl₃) δ 8.63 (s, 1H), 8.54 (d, J = 8.6 Hz, |
| | | [M + H]⁺ calcd for | 1H), 8.20 (d, J = 1.9 Hz, 1H), 8.10 (d, J = 9.0 Hz, 1H), 7.90 (d, |
| | | C₃₀H₂₇ClF₃N₇O₂S, | J = 8.5 Hz, 2H), 7.85-7.65 (m, 2H), 7.24 (s, 2H), 6.83 (dd, J = |
| | | 642.166; found, | 8.7, 2.7 Hz, 1H), 6.40 (d, J = 2.7 Hz, 1H), 3.98 (d, J = 1.3 Hz, |
| | | 642.1665 | 2H), 2.97 (s, 6H), 2.32 (td, J = 7.3, 1.9 Hz, 2H), 1.66-1.54 (m, |
| | | | 2H), 0.91 (t, J = 7.3 Hz, 3H); |
| | | | ¹⁹F NMR (376 MHz, CDCl₃) δ −62.51 |
| J236 | | HRMS-ESI (m/z) | ¹H NMR (400 MHz, CDCl₃) δ 9.13 (s, 1H), 8.48 (td, J = 8.3, 4.8 |
| | | [M + H]⁺ calcd for | Hz, 1H), 8.40 (d, J = 2.7 Hz, 1H), 8.02 (dd, J = 22.8, 8.8 Hz, |
| | | C₂₈H₂₃F₄N₇O₄S, | 2H), 7.94-7.83 (m, 1H), 7.83-7.70 (m, 1H), 7.60-7.45 (m, |
| | | 630.1541; found, | 2H), 7.40 (dd, J = 12.2, 7.8 Hz, 2H), 6.93 (s, 1H), 4.27-4.09 |
| | | 630.1543 | (m, 1H), 4.06-3.92 (m, 2H), 3.15 (d, J = 22.3 Hz, 3H), 2.43 (s, |
| | | | 3H), 1.37 (dd, J = 16.5, 6.4 Hz, 3H); |
| | | | ¹⁹F NMR (376 MHz, CDCl₃) δ −58.38, −131.52 |
| J237 | | HRMS-ESI (m/z) | ¹H NMR (400 MHz, CDCl₃) δ 9.13 (s, 1H), 8.49 (t, J = 8.4 Hz, |
| | | [M + H]⁺ calcd for | 1H), 8.40 (d, J = 2.7 Hz, 1H), 8.02 (ddd, J = 23.6, 8.7, 1.1 Hz, |
| | | C₂₈H₂₀F₇N₇O₄S, | 2H), 7.90 (dd, J = 12.0, 1.8 Hz, 1H), 7.84-7.70 (m, 1H), 7.47 |
| | | 684.1258; found, | (d, J = 3.1 Hz, 1H), 7.45-7.30 (m, 2H), 7.12-6.96 (m, 1H), |
| | | 684.1261 | 4.71-4.41 (m, 2H), 4.04-3.86 (m, 2H), 3.72 (qd, J = 8.7, 4.0 |
| | | | Hz, 2H), 2.45 (s, 3H); ¹⁹F NMR (376 MHz, CDCl₃) |
| | | | δ −58.38, −73.78, −131.35 |
| J238 | | HRMS-ESI (m/z) | ¹H NMR (400 MHz, CDCl₃) δ 9.13 (s, 1H), 8.50 (t, J = 8.4 Hz, |
| | | [M + H]⁺ calcd for | 1H), 8.40 (d, J = 2.7 Hz, 1H), 8.05 (d, J = 8.9 Hz, 1H), 7.98 (dd, |
| | | C₂₉H₂₆F₄N₈O₃S, | J = 8.4, 1.6 Hz, 1H), 7.90 (dd, J = 11.9, 1.9 Hz, 1H), 7.83- |
| | | 643.1857; found, | 7.72 (m, 1H), 7.56 (d, J = 3.0 Hz, 1H), 7.33 (d, J = 8.8 Hz, 1H), |
| | | 643.1866 | 6.89 (dd, J = 8.8, 2.8 Hz, 1H), 6.36 (d, J = 2.7 Hz, 1H), 3.99 (d, |
| | | | J = 1.7 Hz, 2H), 2.97 (s, 6H), 2.59 (hept, J = 6.8 Hz, 1H), 1.17 |
| | | | (dd, J = 9.3, 6.8 Hz, 6H); |
| | | | ¹⁹F NMR (376 MHz, CDCl₃) δ −58.38, −130.71 |
| J239 | | HRMS-ESI (m/z) | ¹H NMR (400 MHz, CDCl₃) δ 9.13 (s, 1H), 8.49 (t, J = 8.3 Hz, |
| | | [M + H]⁺ calcd for | 1H), 8.40 (d, J = 2.7 Hz, 1H), 8.05 (dd, J = 8.9, 0.7 Hz, 1H), |
| | | C₂₈H₂₃F₄N₇O₄S, | 7.99 (dd, J = 8.6, 1.6 Hz, 1H), 7.90 (dd, J = 12.0, 1.8 Hz, 1H), |
| | | 630.1541; found, | 7.83-7.73 (m, 1H), 7.58-7.44 (m, 1H), 7.40 (d, J = 8.8 Hz, |
| | | 630.1544 | 1H), 7.08 (dd, J = 8.7, 2.7 Hz, 1H), 6.61 (d, J = 2.7 Hz, 1H), |
| | | | 4.00 (d, J = 2.0 Hz, 2H), 3.83 (s, 3H), 2.62 (h, J = 6.8 Hz, 1H), |
| | | | 1.19 (t, J = 6.5 Hz, 6H); |
| | | | ¹⁹F NMR (376 MHz, CDCl₃) δ −58.38, −130.99 |
| J240 | | HRMS-ESI (m/z) | ¹H NMR (400 MHz, CDCl₃) δ 9.13 (d, J = 0.6 Hz, 1H), 8.40 (d, |
| | | [M + H]⁺ calcd for | J = 2.7 Hz, 1H), 8.19 (dd, J = 8.4, 3.2 Hz, 1H), 8.12-7.95 (m, |
| | | C₂₉H₂₆F₃N₇O₄S, | 3H), 7.88-7.72 (m, 1H), 7.54 (dd, J = 8.0, 3.1 Hz, 1H), 7.46- |
| | | 626.1792; found, | 7.32 (m, 1H), 7.13-6.88 (m, 2H), 4.25-4.07 (m, 1H), 4.06- |
| | | 626.1795 | 3.92 (m, 2H), 3.17 (d, J = 38.7 Hz, 3H), 2.43 (d, J = 1.9 Hz, |
| | | | 3H), 2.23 (d, J = 23.8 Hz, 3H), 1.38 (dd, J = 26.3, 6.4 Hz, |
| | | | 3H); ¹⁹F NMR (376 MHz, CDCl₃) δ −58.39 |
| J241 | | HRMS-ESI (m/z) | ¹H NMR (400 MHz, CDCl₃) δ 9.13 (s, 1H), 8.40 (d, J = 2.7 Hz, |
| | | [M + H]⁺ calcd for | 1H), 8.20 (d, J = 8.5 Hz, 1H), 8.14-7.93 (m, 3H), 7.77 (d, J = |
| | | C₂₉H₂₃F₆N₇O₄S, | 8.7 Hz, 1H), 7.46-7.29 (m, 2H), 7.05 (d, J = 6.2 Hz, 2H), 4.69- |
| | | 680.1509; found, | 4.45 (m, 2H), 3.94 (d, J = 2.1 Hz, 2H), 3.73 (q, J = 8.7 Hz, |
| | | 680.1512 | 2H), 2.45 (s, 3H), 2.27 (s, 3H); |
| | | | ¹⁹F NMR (376 MHz, CDCl₃) δ −58.39, −73.76 |
| J242 | | HRMS-ESI (m/z) | ¹H NMR (400 MHz, CDCl₃) δ 8.62-8.55 (m, 2H), 8.42-8.32 |
| | | [M + H]⁺ calcd for | (m, 2H), 7.83-7.75 (m, 3H), 7.45-7.37 (m, 2H), 7.32 (dd, J = |
| | | C₃₁H₂₇F₃N₈O₃S, | 8.8, 4.7 Hz, 1H), 6.88 (dd, J = 8.8, 2.7 Hz, 1H), 6.33 (d, J = 2.7 |
| | | 649.1952; found, | Hz, 1H), 4.01 (d, J = 1.0 Hz, 2H), 2.97 (s, 6H), 2.61-2.51 (m, |
| | | 649.195 | 1H), 1.18 (dd, J = 21.9, 6.9 Hz, 6H); |
| | | | ¹⁹F NMR (376 MHz, CDCl₃) δ −58.02 |
| J243 | | HRMS-ESI (m/z) | ¹H NMR (400 MHz, CDCl₃) δ 8.62 (d, J = 8.9 Hz, 1H), 8.56 (s, |
| | | [M + H]⁺ calcd for | 1H), 8.40-8.35 (m, 2H), 7.82-7.76 (m, 2H), 7.74 (s, 1H), |
| | | C₃₀H₂₁F₆N₇O₄S, | 7.42-7.37 (m, 2H), 7.29 (s, 1H), 7.02-6.95 (m, 2H), 4.30- |
| | | 690.1353; found, | 4.18 (m, 2H), 3.95 (d, J = 1.2 Hz, 2H), 2.55 (qt, J = 10.5, 6.1 |
| | | 690.1356 | Hz, 2H), 2.38 (s, 3H); ¹⁹F NMR (376 MHz, CDCl₃) δ −58.02, −64.54 |
| J244 | | HRMS-ESI (m/z) | ¹H NMR (400 MHz, CDCl₃) δ 8.62 (d, J = 8.9 Hz, 1H), 8.56 (s, |
| | | [M + H]⁺ calcd for | 1H), 8.40-8.35 (m, 2H), 7.78 (dd, J = 9.2, 7.0 Hz, 2H), 7.73 |
| | | C₂₈H₁₇F₆N₇O₃S, | (d, J = 8.6 Hz, 2H), 7.51-7.45 (m, 1H), 7.42-7.38 (m, 2H), |
| | | 646.1091; found, | 7.12 (s, 1H), 4.09-3.91 (m, 2H), 2.52 (s, 3H); |
| | | 646.1083 | ¹⁹F NMR (376 MHz, CDCl₃) δ −58.02, −61.07 |
| J245 | | HRMS-ESI (m/z) | ¹H NMR (400 MHz, CDCl₃) δ 8.61 (d, J = 9.3 Hz, 1H), 8.56 (s, |
| | | [M + H]⁺ calcd for | 1H), 8.40-8.36 (m, 2H), 7.81-7.76 (m, 2H), 7.71 (s, 1H), |
| | | C₃₀H₂₁F₆N₇O₄S, | 7.42-7.37 (m, 3H), 7.34 (dd, J = 7.4, 1.5 Hz, 1H), 7.05-7.03 |
| | | 690.1353; found, | (m, 1H), 4.63 (d, J = 12.4 Hz, 1H), 4.50 (d, J = 12.5 Hz, 1H), |
| | | 690.1346 | 3.97 (d, J = 3.1 Hz, 2H), 3.77-3.65 (m, 2H), 2.45 (s, 3H); ¹⁹F |
| | | | NMR (376 MHz, CDCl₃) δ −58.01, −73.75 |
| J246 | | HRMS-ESI (m/z) | ¹H NMR (400 MHz, CDCl₃) δ 8.56 (s, 1H), 8.38 (d, J = 9.1 Hz, |
| | | [M + H]⁺ calcd for | 2H), 7.81-7.75 (m, 3H), 7.40 (d, J = 8.7 Hz, 3H), 7.08 (dd, J = |
| | | C₃₀H₂₄F₃N₇O₄S, | 8.7, 2.7 Hz, 1H), 6.60 (d, J = 2.7 Hz, 1H), 4.02 (d, J = 1.1 Hz, |
| | | 636.1635; found, | 2H), 3.83 (s, 3H), 2.66-2.56 (m, 1H), 1.20 (dd, J = 20.2, 6.9 |
| | | 636.1636 | Hz, 6H); ¹⁹F NMR (376 MHz, CDCl₃) δ −58.01 |
| J247 | | HRMS-ESI (m/z) | ¹H NMR (400 MHz, CDCl₃) δ 8.59 (d, J = 8.9 Hz, 1H), 8.56 (s, |
| | | [M + H]⁺ calcd for | 1H), 8.37 (d, J = 10.0 Hz, 2H), 7.83-7.76 (m, 3H), 7.40 (d, J = |
| | | C₃₁H₂₇F₃N₈O₃S, | 8.6 Hz, 2H), 7.24 (d, J = 8.7 Hz, 1H), 6.82 (dd, J = 8.7, 2.7 Hz, |
| | | 649.1952; found, | 1H), 6.37 (d, J = 2.7 Hz, 1H), 4.00 (d, J = 1.5 Hz, 2H), 2.97 (s, |
| | | 649.1954 | 6H), 2.35-2.26 (m, 2H), 1.61-1.51 (m, 2H), 0.94-0.88 (m, |
| | | | 3H); ¹⁹F NMR (376 MHz, CDCl₃) δ −58.01 |
| J248 | | HRMS-ESI (m/z) | ¹H NMR (400 MHz, CDCl₃) δ 8.60 (d, J = 8.8 Hz, 1H), 8.56 (s, |
| | | [M + H]⁺ calcd for | 1H), 8.41-8.35 (m, 2H), 7.78 (dd, J = 7.9, 5.7 Hz, 3H), 7.40 |
| | | C₃₀H₂₄F₃N₇O₃S, | (d, J = 8.5 Hz, 2H), 7.32-7.27 (m, 1H), 6.91 (s, 1H), 4.00 (d, J = |
| | | 620.1686; found, | 2.0 Hz, 2H), 2.41-2.34 (m, 5H), 1.60 (dd, J = 15.5, 7.8 Hz, |
| | | 620.168 | 2H), 0.93 (t, J = 7.3 Hz, 3H); ¹⁹F NMR (376 MHz, CDCl₃) δ −58.01 |
| J249 | | HRMS-ESI (m/z) | ¹H NMR (400 MHz, CDCl₃) δ 9.14 (s, 1H), 8.54 (d, J = 8.7 Hz, |
| | | [M + H]⁺ calcd for | 1H), 8.40 (d, J = 2.8 Hz, 1H), 8.20 (d, J = 1.9 Hz, 1H), 8.08 (dd, |
| | | C₂₈H₂₀ClF₆N₇O₄S, | J = 19.5, 8.5 Hz, 2H), 7.78 (d, J = 8.7 Hz, 1H), 7.69 (s, 1H), |
| | | 700.0963; found, | 7.42 (d, J = 7.8 Hz, 1H), 7.35 (d, J = 7.8 Hz, 1H), 7.05 (d, J = |
| | | 700.0961 | 1.3 Hz, 1H), 4.64 (d, J = 12.5 Hz, 1H), 4.51 (d, J = 12.5 Hz, |
| | | | 1H), 4.05-3.86 (m, 2H), 3.72 (qd, J = 8.7, 3.7 Hz, 2H), 2.45 |
| | | | (s, 3H); ¹⁹F NMR (376 MHz, CDCl₃) δ −58.38, −73.75 |
| J250 | | HRMS-ESI (m/z) | ¹H NMR (400 MHz, CDCl₃) δ 9.14 (d, J = 0.7 Hz, 1H), 8.52 (d, |
| | | [M + H]⁺ calcd for | J = 8.6 Hz, 1H), 8.40 (d, J = 2.8 Hz, 1H), 8.20 (dd, J = 4.9, 1.9 |
| | | C₂₈H₂₃ClF₃N₇O₄S, | Hz, 1H), 8.17-7.99 (m, 2H), 7.84-7.75 (m, 1H), 7.69 (d, J = |
| | | 646.1246; found, | 20.4 Hz, 1H), 7.54 (d, J = 8.0 Hz, 1H), 7.38 (d, J = 8.0 Hz, 1H), |
| | | 646.124 | 6.93 (s, 1H), 4.16 (dq, J = 18.9, 6.4 Hz, 1H), 4.00 (d, J = 1.4 |
| | | | Hz, 2H), 3.17 (d, J = 40.8 Hz, 3H), 2.51-2.33 (m, 3H), 1.38 |
| | | | (dd, J = 30.7, 6.5 Hz, 3H); |
| | | | ¹⁹F NMR (376 MHz, CDCl₃) δ −58.38 |
| J251 | | HRMS-ESI (m/z) | ¹H NMR (400 MHz, CDCl₃) δ 9.13 (s, 1H), 8.40 (d, J = 2.8 Hz, |
| | | [M + H]⁺ calcd for | 1H), 8.20 (d, J = 8.5 Hz, 1H), 8.13-7.94 (m, 2H), 7.77 (ddd, J = |
| | | C₃₀H₂₉F₃N₈O₃S, | 8.9, 2.8, 1.1 Hz, 1H), 7.33 (d, J = 8.8 Hz, 1H), 7.13 (s, 1H), |
| | | 639.2108; found, | 6.89 (dd, J = 8.8, 2.8 Hz, 1H), 6.38 (d, J = 2.7 Hz, 1H), 3.97 (s, |
| | | 639.2106 | 2H), 2.97 (s, 6H), 2.61 (p, J = 6.8 Hz, 2H), 2.27 (s, 3H), 1.18 |
| | | | (dd, J = 15.7, 6.9 Hz, 6H); |
| | | | ¹⁹F NMR (376 MHz, CDCl₃) δ −58.39 |
| J252 | | HRMS-ESI (m/z) | ¹H NMR (400 MHz, CDCl₃) δ 9.13 (s, 1H), 8.40 (d, J = 2.8 Hz, |
| | | [M + H]⁺ calcd for | 1H), 8.20 (d, J = 8.5 Hz, 1H), 8.12-7.96 (m, 3H), 7.86-7.70 |
| | | C₂₉H₂₆F₃N₇O₄S, | (m, 1H), 7.39 (d, J = 8.8 Hz, 1H), 7.13-6.98 (m, 2H), 6.63 (d, |
| | | 626.1792; found, | J = 2.7 Hz, 1H), 3.98 (d, J = 0.9 Hz, 2H), 3.83 (s, 3H), 2.65 (p, |
| | | 626.1792 | J = 6.9 Hz, 2H), 2.27 (s, 3H), 1.19 (dd, J = 12.3, 6.9 Hz, 6H); ¹⁹F |
| | | | NMR (376 MHz, CDCl₃) δ −58.39 |
| J253 | | | ¹H NMR (400 MHz, CDCl₃) δ 9.14 (s, 1H), 8.53 (d, J = 8.7 Hz, |
| | | | 1H), 8.40 (d, J = 2.7 Hz, 1H), 8.21 (d, J = 1.9 Hz, 1H), 8.17- |
| | | | 7.99 (m, 2H), 7.86-7.68 (m, 2H), 7.40 (d, J = 8.8 Hz, 2H), |
| | | | 7.07 (dd, J = 8.8, 2.7 Hz, 2H), 6.62 (d, J = 2.7 Hz, 1H), 4.08- |
| | | | 3.93 (m, 2H), 3.83 (s, 3H), 2.64 (p, J = 6.8 Hz, 2H), 1.19 (dd, J = |
| | | | 16.2, 6.9 Hz, 6H); ¹⁹F NMR (376 MHz, CDCl₃) δ −58.38 |
| J254 | | HRMS-ESI (m/z) | ¹H NMR (400 MHz, CDCl₃) δ 9.13 (s, 1H), 8.40 (d, J = 2.7 Hz, |
| | | [M + H]⁺ calcd for | 1H), 8.21 (d, J = 8.5 Hz, 1H), 8.14-7.93 (m, 3H), 7.83-7.72 |
| | | C₃₀H₂₉F₃N₈O₃S, | (m, 1H), 7.13 (s, 1H), 6.82 (dd, J = 8.6, 2.8 Hz, 1H), 6.42 (d, J = |
| | | 639.2108; found, | 2.7 Hz, 1H), 3.96 (d, J = 1.4 Hz, 2H), 2.97 (s, 6H), 2.40- |
| | | 639.2118 | 2.19 (m, 6H), 1.57 (h, J = 7.4 Hz, 2H), 0.92 (t, J = 7.3 Hz, 3H); |
| | | | ¹⁹F NMR (376 MHz, CDCl₃) δ −58.39 |
| J255 | | HRMS-ESI (m/z) | ¹H NMR (400 MHz, CDCl₃) δ 9.13 (s, 1H), 8.40 (d, J = 2.8 Hz, |
| | | [M + H]⁺ calcd for | 1H), 8.22 (d, J = 8.5 Hz, 1H), 8.14-7.96 (m, 3H), 7.77 (dd, J = |
| | | C₂₉H₂₃F₆N₇O₄S, | 8.6, 2.7 Hz, 1H), 7.13-7.01 (m, 2H), 6.97 (d, J = 8.5 Hz, 1H), |
| | | 680.1509; found, | 4.23 (t, J = 6.3 Hz, 2H), 3.92 (d, J = 1.8 Hz, 2H), 2.55 (dtt, J = |
| | | 680.1516 | 16.0, 10.7, 6.1 Hz, 2H), 2.33 (d, J = 40.6 Hz, 6H); ¹⁹F NMR |
| | | | (376 MHz, CDCl₃) δ −58.39, −64.57 |
| J256 | 115-120 | HRMS-ESI (m/z) | ¹H NMR (400 MHz, CDCl₃) δ 8.54 (s, 1H), 8.47 (td, J = 8.4, 4.5 |
| | | [M + H]⁺ calcd for | Hz, 1H), 7.97 (d, J = 8.7 Hz, 1H), 7.89 (dd, J = 11.4, 2.9 Hz, |
| | | C₂₉H₂₅F₄N₆O₅S, | 1H), 7.82-7.73 (m, 2H), 7.55 (dd, J = 8.8, 3.6 Hz, 1H), 7.52- |
| | | 645.1538; found, | 7.42 (m, 1H), 7.39 (d, J = 8.6 Hz, 2H), 7.14-7.08 (m, 1H), |
| | | 645.1536 | 6.64 (d, J = 2.7 Hz, 1H), 4.20-4.07 (m, 1H), 4.03-3.96 (m, |
| | | | 2H), 3.85 (s, 3H), 3.18-3.10 (m, 3H), 1.37 (dd, J = 12.4, 6.4 |
| | | | Hz, 3H); ¹⁹F NMR (376 MHz, CDCl₃) δ −58.03, −131.11, −131.47 |
| J257 | 198-203 | | ¹H NMR (300 MHz, CDCl₃) δ 9.13 (d, J = 0.7 Hz, 1H), 8.51 (t, J = |
| | | | 8.4 Hz, 1H), 8.40 (d, J = 2.7 Hz, 1H), 8.12-8.02 (m, 1H), |
| | | | 7.99 (d, J = 8.6 Hz, 1H), 7.90 (dd, J = 12.1, 1.8 Hz, 1H), 7.78 |
| | | | (d, J = 8.9 Hz, 1H), 7.52 (s, 1H), 7.32 (d, J = 7.9 Hz, 1H), 7.07- |
| | | | 6.92 (m, 2H), 4.31-4.16 (m, 2H), 3.94 (s, 2H), 2.54 (ddq, J = |
| | | | 16.2, 10.2, 5.8 Hz, 2H), 2.38 (s, 3H) |
| J258 | | | ¹H NMR (400 MHz, CDCl₃) δ 9.13 (s, 1H), 8.51 (t, J = 8.3 Hz, |
| | | | 1H), 8.40 (d, J = 2.7 Hz, 1H), 8.05 (d, J = 8.9 Hz, 1H), 7.98 (dd, |
| | | | J = 8.8, 1.5 Hz, 1H), 7.90 (dd, J = 12.0, 1.9 Hz, 1H), 7.84- |
| | | | 7.68 (m, 1H), 7.56 (d, J = 3.0 Hz, 1H), 7.24 (s, 1H), 6.83 (dd, J = |
| | | | 8.6, 2.7 Hz, 1H), 6.40 (d, J = 2.7 Hz, 1H), 3.98 (d, J = 1.9 Hz, |
| | | | 2H), 2.97 (s, 6H), 2.41-2.18 (m, 2H), 1.64-1.47 (m, 2H), |
| | | | 0.91 (t, J = 7.3 Hz, 3H); ¹⁹F NMR (376 MHz, CDCl₃) δ −58.38, −130.76 |
| J259 | | | ¹H NMR (300 MHz, CDCl₃) δ 9.16 (s, 1H), 8.59 (d, J = 8.8 Hz, |
| | | | 1H), 8.44-8.36 (m, 3H), 8.05 (dd, J = 8.9, 0.6 Hz, 1H), 7.84- |
| | | | 7.75 (m, 2H), 7.39 (d, J = 8.1 Hz, 1H), 7.36-7.30 (m, 1H), |
| | | | 6.90 (d, J = 9.0 Hz, 1H), 4.01 (d, J = 0.9 Hz, 2H), 2.64 (p, J = |
| | | | 6.9 Hz, 1H), 2.39 (s, 3H), 1.21 (dd, J = 16.7, 6.8 Hz, 6H) |
| J260 | | HRMS-ESI (m/z) | ¹H NMR (300 MHz, CDCl₃) δ 9.17 (d, J = 0.4 Hz, 1H), 8.41 (d, |
| | | [M + H]⁺ calcd for | J = 3.0 Hz, 2H), 8.35 (d, J = 9.0 Hz, 1H), 8.17-8.11 (m, 1H), |
| | | C₂₉H₂₃F₆N₇O₃S, | 8.11-8.04 (m, 1H), 7.83-7.77 (m, 1H), 7.55 (s, 1H), 7.38 (d, |
| | | 664.156; found, | J = 8.0 Hz, 1H), 7.31 (d, J = 8.1 Hz, 1H), 6.91-6.86 (m, 1H), |
| | | 664.1557 | 4.13-3.84 (m, 2H), 2.64 (q, J = 7.1 Hz, 1H), 2.38 (s, 3H), 1.24- |
| | | | 1.16 (m, 6H) |
| J261 | | HRMS-ESI (m/z) | ¹H NMR (300 MHz, CDCl₃) δ 9.14 (s, 1H), 8.49 (t, J = 8.4 Hz, |
| | | [M + H]⁺ calcd for | 1H), 8.40 (d, J = 2.7 Hz, 1H), 8.05 (dd, J = 8.9, 0.7 Hz, 1H), |
| | | C₂₉H₂₅F₅N₈O₃S, | 7.99 (d, J = 8.7 Hz, 1H), 7.91 (dd, J = 12.0, 1.8 Hz, 1H), 7.78 |
| | | 661.1763; found, | (d, J = 9.3 Hz, 1H), 7.55 (d, J = 3.0 Hz, 1H), 7.09 (d, J = 14.1 |
| | | 661.1769 | Hz, 1H), 6.53 (d, J = 8.3 Hz, 1H), 3.99 (d, J = 1.0 Hz, 2H), 2.87 |
| | | | (d, J = 0.8 Hz, 6H), 2.57 (q, J = 6.7 Hz, 1H), 1.17 (dd, J = 6.8, |
| | | | 4.9 Hz, 6H) |
| J262 | | HRMS-ESI (m/z) | ¹H NMR (300 MHz, CDCl₃) δ 9.13 (s, 1H), 8.51 (t, J = 8.4 Hz, |
| | | [M + H]⁺ calcd for | 1H), 8.40 (d, J = 2.7 Hz, 1H), 8.11-7.94 (m, 2H), 7.90 (dd, J = |
| | | C₂₇H₁₉F₇N₈O₃S, | 12.1, 1.9 Hz, 1H), 7.85-7.72 (m, 1H), 7.63 (d, J = 9.0 Hz, 1H), |
| | | 669.1262; found, | 7.54 (d, J = 3.1 Hz, 1H), 6.80 (dd, J = 8.8, 2.6 Hz, 1H), 6.44 (d, |
| | | 669.1263 | J = 2.6 Hz, 1H), 4.11-3.81 (m, 2H), 3.08 (s, 6H) |
| J263 | | HRMS-ESI (m/z) | ¹H NMR (300 MHz, CDCl₃) δ 9.13 (s, 1H), 8.51 (t, J = 8.4 Hz, |
| | | [M + H]⁺ calcd for | 1H), 8.40 (d, J = 2.8 Hz, 1H), 8.02 (dd, J = 19.6, 8.8 Hz, 2H), |
| | | C₂₈H₂₁F₇N₈O₄S, | 7.90 (dd, J = 12.0, 1.8 Hz, 1H), 7.78 (d, J = 9.2 Hz, 1H), 7.54 |
| | | 699.1367; found, | (d, J = 3.1 Hz, 1H), 7.04 (d, J = 9.1 Hz, 1H), 6.82 (dd, J = 9.2, |
| | | 699.1377 | 3.0 Hz, 1H), 6.54 (d, J = 3.1 Hz, 1H), 4.40-4.18 (m, 2H), 3.96 |
| | | | (d, J = 3.1 Hz, 2H), 2.97 (s, 6H) |
| J264 | | HRMS-ESI (m/z) | ¹H NMR (300 MHz, CDCl₃) δ 9.16 (s, 1H), 8.64 (d, J = 8.9 Hz, |
| | | [M + H]⁺ calcd for | 1H), 8.43-8.37 (m, 3H), 8.05 (dd, J = 8.9, 0.7 Hz, 1H), 7.82- |
| | | C₂₉H₂₀F₆N₈O₄S, | 7.73 (m, 2H), 7.31-7.27 (m, 1H), 7.04-6.94 (m, 2H), 4.33- |
| | | 691.1305; found, | 4.15 (m, 2H), 3.95 (d, J = 0.7 Hz, 2H), 2.65-2.48 (m, 2H), |
| | | 691.1311 | 2.38 (s, 3H) |
| J265 | | HRMS-ESI (m/z) | ¹H NMR (300 MHz, CDCl₃) δ 9.16 (s, 1H), 8.63 (d, J = 9.3 Hz, |
| | | [M + H]⁺ calcd for | 1H), 8.41 (d, J = 4.2 Hz, 3H), 8.05 (d, J = 8.9 Hz, 1H), 7.86- |
| | | C₂₉H₂₀F₆N₈O₄S, | 7.76 (m, 1H), 7.72 (s, 1H), 7.42-7.31 (m, 2H), 7.04 (s, 1H), |
| | | 691.1305; found, | 4.64 (d, J = 12.5 Hz, 1H), 4.50 (d, J = 12.5 Hz, 1H), 3.97 (d, J = |
| | | 691.1311 | 1.9 Hz, 2H), 3.71 (qd, J = 8.7, 3.3 Hz, 2H), 2.46 (s, 3H) |
| J266 | | HRMS-ESI (m/z) | ¹H NMR (300 MHz, CDCl₃) δ 9.15 (s, 1H), 8.61-8.56 (m, 1H), |
| | | [M + H]⁺ calcd for | 8.44-8.36 (m, 3H), 8.05 (dd, J = 9.0, 0.7 Hz, 1H), 7.85-7.77 |
| | | C₃₀H₂₆F₃N₉O₃S, | (m, 2H), 7.32 (dd, J = 8.8, 3.3 Hz, 1H), 6.91-6.87 (m, 1H), |
| | | 650.1904; found, | 6.34 (d, J = 2.7 Hz, 1H), 4.01 (s, 2H), 2.97 (s, 6H), 2.56 (p, J = |
| | | 650.1916 | 6.8 Hz, 1H), 1.23-1.13 (m, 6H) |
| J267 | | HRMS-ESI (m/z) | ¹H NMR (300 MHz, CDCl₃) δ 9.16 (d, J = 0.6 Hz, 1H), 8.59 (d, |
| | | [M + H]⁺ calcd for | J = 9.5 Hz, 1H), 8.44-8.36 (m, 3H), 8.07-8.02 (m, 1H), 7.80 |
| | | C₂₉H₂₃F₃N₈O₄S, | (d, J = 6.7 Hz, 1H), 7.72 (d, J = 16.4 Hz, 1H), 7.53 (d, J = 8.0 |
| | | 637.1588; found, | Hz, 1H), 7.39 (d, J = 8.3 Hz, 1H), 6.91 (d, J = 1.7 Hz, 1H), 4.15 |
| | | 637.159 | (dd, J = 11.3, 6.4 Hz, 1H), 4.02 (s, 2H), 3.17 (d, J = 30.9 Hz, |
| | | | 3H), 2.43 (s, 3H), 1.38 (dd, J = 23.0, 6.4 Hz, 3H) |
| J268 | | HRMS-ESI (m/z) | ¹H NMR (300 MHz, CDCl₃) δ 9.16 (s, 1H), 8.60 (d, J = 8.8 Hz, |
| | | [M + H]⁺ calcd for | 1H), 8.41 (d, J = 3.5 Hz, 3H), 8.05 (d, J = 8.9 Hz, 1H), 7.84- |
| | | C₂₉H₂₃F₃N₈O₄S, | 7.76 (m, 2H), 7.40 (d, J = 8.7 Hz, 1H), 7.08 (dd, J = 8.8, 2.7 Hz, |
| | | 637.1588; found, | 1H), 6.60 (d, J = 2.7 Hz, 1H), 4.02 (s, 2H), 3.83 (s, 3H), 2.61 |
| | | 637.1591 | (dt, J = 13.6, 6.4 Hz, 1H), 1.20 (dd, J = 15.0, 6.8 Hz, 6H) |
| J269 | | HRMS-ESI (m/z) | ¹H NMR (300 MHz, CDCl₃) δ 9.16 (s, 1H), 8.61 (d, J = 8.7 Hz, |
| | | [M + H]⁺ calcd for | 1H), 8.39 (d, J = 10.4 Hz, 3H), 8.05 (d, J = 8.7 Hz, 1H), 7.80 (d, |
| | | C₃₀H₂₆F₃N₉O₃S, | J = 8.4 Hz, 2H), 7.23 (s, 1H), 6.82 (dd, J = 8.7, 2.7 Hz, 1H), |
| | | 650.1904; found, | 6.37 (d, J = 2.7 Hz, 1H), 4.00 (s, 2H), 2.97 (s, 6H), 2.36-2.27 |
| | | 650.1912 | (m, 2H), 1.58 (s, 2H), 0.92 (t, J = 7.3 Hz, 3H) |
| J270 | | HRMS-ESI (m/z) | ¹H NMR (300 MHz, CDCl₃) δ 9.13 (s, 1H), 8.49 (t, J = 8.4 Hz, |
| | | [M + H]⁺ calcd for | 1H), 8.40 (d, J = 2.7 Hz, 1H), 8.10-7.96 (m, 2H), 7.90 (dd, J = |
| | | C₂₈H₂₃F₄N₇O₃S, | 11.9, 1.8 Hz, 1H), 7.78 (d, J = 10.2 Hz, 1H), 7.53 (d, J = 3.0 |
| | | 614.1592; found, | Hz, 1H), 7.46-7.32 (m, 2H), 6.91 (s, 1H), 4.00 (d, J = 1.4 Hz, |
| | | 614.1595 | 2H), 2.66 (p, J = 7.0 Hz, 1H), 2.39 (s, 3H), 1.20 (t, J = 6.8 Hz, |
| | | | 6H) |
| J271 | | HRMS-ESI (m/z) | ¹H NMR (300 MHz, CDCl₃) δ 9.13 (s, 1H), 8.50 (t, J = 8.4 Hz, |
| | | [M + H]⁺ calcd for | 1H), 8.40 (d, J = 2.7 Hz, 1H), 8.13-7.94 (m, 2H), 7.90 (dd, J = |
| | | C₂₆H₁₆F₇N₇O₃S, | 12.1, 1.8 Hz, 1H), 7.85-7.67 (m, 2H), 7.47 (d, J = 9.1 Hz, 2H), |
| | | 640.0996; found, | 7.12 (s, 1H), 4.12-3.86 (m, 2H), 2.51 (s, 3H) |
| | | 640.1006 | |
| J272 | | HRMS-ESI (m/z) | ¹H NMR (300 MHz, CDCl₃) δ 9.14 (s, 1H), 8.50 (t, J = 8.4 Hz, |
| | | [M + H]⁺ calcd for | 1H), 8.40 (d, J = 2.7 Hz, 1H), 8.10-7.95 (m, 2H), 7.90 (dd, J = |
| | | C₂₈H₂₃F₄N₇O₃S, | 12.0, 1.8 Hz, 1H), 7.85-7.72 (m, 1H), 7.53 (d, J = 3.1 Hz, 1H), |
| | | 614.1592; found, | 7.31 (d, J = 8.1 Hz, 2H), 6.93 (s, 1H), 3.99 (d, J = 1.5 Hz, 2H), |
| | | 614.1597 | 2.42-2.34 (m, 5H), 1.64-1.56 (m, 2H), 0.93 (t, J = 7.3 Hz, |
| | | | 3H) |
| J273 | | HRMS-ESI (m/z) | ¹H NMR (300 MHz, CDCl₃) δ 9.16 (s, 1H), 8.50 (d, J = 8.7 Hz, |
| | | [M + H]⁺ calcd for | 1H), 8.42 (s, 2H), 8.41-8.33 (m, 1H), 8.08 (d, J = 8.9 Hz, 1H), |
| | | C₂₉H₂₀F₉N₇O₄S, | 7.79 (dd, J = 8.9, 2.4 Hz, 1H), 7.54 (s, 1H), 7.30 (s, 1H), 7.03 |
| | | 734.1227; found, | (s, 1H), 6.96 (d, J = 8.4 Hz, 1H), 4.22 (t, J = 6.0 Hz, 2H), 3.94 |
| | | 734.1224 | (d, J = 0.8 Hz, 2H), 2.61-2.47 (m, 2H), 2.38 (s, 3H) |
| J274 | | HRMS-ESI (m/z) | ¹H NMR (300 MHz, CDCl₃) δ 9.16 (s, 1H), 8.52-8.32 (m, 4H), |
| | | [M + H]⁺ calcd for | 8.08 (dd, J = 9.0, 0.6 Hz, 1H), 7.80 (d, J = 9.5 Hz, 1H), 7.50 (s, |
| | | C₂₉H₂₀F₉N₇O₄S, | 1H), 7.45-7.30 (m, 2H), 7.05 (s, 1H), 4.63 (d, J = 12.7 Hz, |
| | | 734.1227; found, | 1H), 4.51 (d, J = 12.6 Hz, 1H), 3.97 (d, J = 1.3 Hz, 2H), 3.78- |
| | | 734.1229 | 3.58 (m, 2H), 2.45 (s, 3H) |
| J275 | | ESIMS m/z | ¹H NMR (300 MHz, CDCl₃) δ 9.16 (s, 1H), 8.44 (d, J = 11.8 Hz, |
| | | 693 ([M + H]⁺) | 3H), 8.36 (d, J = 9.1 Hz, 1H), 8.08 (dd, J = 8.9, 0.6 Hz, 1H), |
| | | | 7.79 (dd, J = 8.8, 1.9 Hz, 1H), 7.58 (s, 1H), 7.32 (d, J = 8.8 Hz, |
| | | | 1H), 6.88 (dd, J = 8.8, 2.7 Hz, 1H), 6.35 (d, J = 2.7 Hz, 1H), |
| | | | 4.00 (s, 2H), 2.96 (s, 6H), 2.62-2.53 (m, 1H), 1.17 (dd, J = |
| | | | 13.1, 6.8 Hz, 6H) |
| J276 | | ESIMS m/z | ¹H NMR (300 MHz, CDCl₃) δ 9.16 (d, J = 0.6 Hz, 1H), 8.53- |
| | | 680 ([M + H]⁺) | 8.31 (m, 4H), 8.08 (d, J = 8.9 Hz, 1H), 7.79 (d, J = 9.3 Hz, 1H), |
| | | | 7.52 (t, J = 10.3 Hz, 2H), 7.38 (d, J = 8.1 Hz, 1H), 6.96-6.88 |
| | | | (m, 1H), 4.23-4.05 (m, 1H), 4.01 (d, J = 1.5 Hz, 2H), 3.21- |
| | | | 3.07 (m, 3H), 2.42 (s, 3H), 1.30 (dd, J = 21.7, 4.8 Hz, 3H) |
| J277 | | ESIMS m/z | ¹H NMR (300 MHz, CDCl₃) δ 9.16 (s, 1H), 8.49-8.33 (m, 4H), |
| | | 680 ([M + H]⁺) | 8.08 (d, J = 8.9 Hz, 1H), 7.80 (d, J = 9.0 Hz, 1H), 7.56 (s, 1H), |
| | | | 7.39 (d, J = 8.8 Hz, 1H), 7.07 (dd, J = 8.8, 2.7 Hz, 1H), 6.61 (d, |
| | | | J = 2.7 Hz, 1H), 4.01 (s, 2H), 3.82 (s, 3H), 2.69-2.57 (m, 1H), |
| | | | 1.19 (dd, J = 11.7, 6.8 Hz, 6H) |
| J278 | | ESIMS m/z | ¹H NMR (300 MHz, CDCl₃) δ 9.16 (s, 1H), 8.61 (d, J = 8.7 Hz, |
| | | 693 ([M + H]⁺) | 1H), 8.39 (d, J = 10.4 Hz, 3H), 8.05 (d, J = 8.7 Hz, 1H), 7.80 (d, |
| | | | J = 8.4 Hz, 2H), 7.23 (s, 1H), 6.82 (dd, J = 8.7, 2.7 Hz, 1H), |
| | | | 6.37 (d, J = 2.7 Hz, 1H), 4.00 (s, 2H), 2.97 (s, 6H), 2.36-2.27 |
| | | | (m, 2H), 1.58 (s, 2H), 0.92 (t, J = 7.3 Hz, 3H) |
| J279 | | HRMS-ESI (m/z) | ¹H NMR (400 MHz, CDCl₃) δ 9.22 (s, 1H), 8.74 (dd, J = 2.3, 1.1 |
| | | [M + H]⁺ calcd for | Hz, 1H), 8.50 (t, J = 8.4 Hz, 1H), 8.20-8.07 (m, 2H), 8.00 (d, J = |
| | | C₂₈H₂₃F₄N₇O₂S, | 8.9 Hz, 1H), 7.91 (dd, J = 12.0, 1.8 Hz, 1H), 7.52 (s, 1H), |
| | | 598.1643; found, | 7.41-7.30 (m, 2H), 6.90 (s, 1H), 3.99 (d, J = 2.3 Hz, 2H), 2.66 |
| | | 598.1644 | (p, J = 6.9 Hz, 1H), 2.39 (s, 3H), 1.26-1.14 (m, 6H); ¹⁹F NMR |
| | | | (376 MHz, CDCl₃) δ −62.10, −130.94 |
| J280 | | HRMS-ESI (m/z) | ¹H NMR (400 MHz, CDCl₃) δ 9.22 (s, 1H), 8.74 (dd, J = 2.2, 1.2 |
| | | [M + H]⁺ calcd for | Hz, 1H), 8.49 (td, J = 8.4, 4.7 Hz, 1H), 8.20-8.06 (m, 1H), |
| | | C₂₈H₂₃F₄N₇O₃S, | 8.00 (d, J = 8.7 Hz, 1H), 7.91 (ddd, J = 12.0, 3.5, 1.8 Hz, 1H), |
| | | 614.1592; found, | 7.53 (dt, J = 9.8, 4.9 Hz, 2H), 7.40 (dd, J = 13.1, 8.5 Hz, 2H), |
| | | 614.16 | 6.93 (s, 1H), 4.24-4.08 (m, 1H), 4.06-3.93 (m, 2H), 3.16 (d, |
| | | | J = 22.5 Hz, 3H), 2.49-2.37 (m, 3H), 1.37 (dd, J = 16.4, 6.5 |
| | | | Hz, 3H); ¹⁹F NMR (376 MHz, CDCl₃) δ −62.10, −131.45 |
| J281 | | HRMS-ESI (m/z) | ¹H NMR (400 MHz, CDCl₃) δ 9.21 (s, 1H), 8.74 (dt, J = 2.0, 0.9 |
| | | [M + H]⁺ calcd for | Hz, 1H), 8.50 (t, J = 8.3 Hz, 1H), 8.23-8.05 (m, 2H), 8.06- |
| | | C₂₈H₂₀F₇N₇O₃S, | 7.95 (m, 1H), 7.91 (dd, J = 12.0, 1.9 Hz, 1H), 7.52-7.30 (m, |
| | | 668.1309; found, | 3H), 7.04 (d, J = 1.5 Hz, 1H), 4.63 (d, J = 12.4 Hz, 1H), 4.50 (d, |
| | | 668.1317 | J = 12.4 Hz, 1H), 4.04-3.85 (m, 2H), 3.72 (qd, J = 8.7, 3.7 Hz, |
| | | | 2H), 2.45 (s, 3H); ¹⁹F NMR (376 MHz, CDCl₃) |
| | | | δ −62.10, −73.78, −131.28 |
| J282 | | HRMS-ESI (m/z) | ¹H NMR (400 MHz, CDCl₃) δ 9.21 (s, 1H), 8.73 (dt, J = 2.0, 1.0 |
| | | [M + H]⁺ calcd for | Hz, 1H), 8.51 (t, J = 8.4 Hz, 1H), 8.19-8.07 (m, 2H), 8.06- |
| | | C₂₉H₂₆F₄N₈O₂S, | 7.95 (m, 1H), 7.91 (dd, J = 11.9, 1.9 Hz, 1H), 7.58 (d, J = 3.0 |
| | | 627.1908; found, | Hz, 1H), 7.23 (s, 1H), 6.83 (dd, J = 8.7, 2.7 Hz, 1H), 6.40 (d, J = |
| | | 627.1925 | 2.7 Hz, 1H), 3.98 (d, J = 1.9 Hz, 2H), 2.97 (s, 6H), 2.38- |
| | | | 2.22 (m, 2H), 1.55 (q, J = 7.4 Hz, 2H), 0.91 (t, J = 7.3 Hz, 3H); |
| | | | ¹⁹F NMR (376 MHz, CDCl₃) δ −62.09, −130.69 |
| J283 | | HRMS-ESI (m/z) | ¹H NMR (400 MHz, CDCl₃) δ 9.22 (s, 1H), 8.74 (dt, J = 2.0, 0.9 |
| | | [M + H]⁺ calcd for | Hz, 1H), 8.50 (t, J = 8.3 Hz, 1H), 8.18-8.07 (m, 2H), 8.00 (d, J = |
| | | C₂₈H₂₃F₄N₇O₃S, | 8.8 Hz, 1H), 7.92 (dd, J = 11.9, 1.9 Hz, 1H), 7.53 (d, J = 3.0 |
| | | 614.1592; found, | Hz, 1H), 7.40 (d, J = 8.9 Hz, 1H), 7.08 (dd, J = 8.7, 2.7 Hz, 1H), |
| | | 614.1605 | 6.61 (d, J = 2.7 Hz, 1H), 4.00 (d, J = 2.0 Hz, 2H), 3.83 (s, 3H), |
| | | | 2.63 (p, J = 6.9 Hz, 1H), 1.19 (t, J = 6.5 Hz, 6H); ¹⁹F NMR (376 |
| | | | MHz, CDCl₃) δ −62.10, −130.93 |
| J284 | | HRMS-ESI (m/z) | ¹H NMR (400 MHz, CDCl₃) δ 9.20 (d, J = 11.9 Hz, 1H), 8.79- |
| | | [M + H]⁺ calcd for | 8.66 (m, 1H), 8.21 (d, J = 8.5 Hz, 1H), 8.17-8.05 (m, 3H), |
| | | C₂₉H₂₆F₃N₇O₂S, | 8.01 (s, 1H), 7.44-7.29 (m, 2H), 7.10 (s, 1H), 6.92 (s, 1H), |
| | | 594.1894; found, | 3.98 (d, J = 0.9 Hz, 2H), 2.69 (p, J = 6.8 Hz, 1H), 2.39 (s, 3H), |
| | | 594.1901 | 2.26 (d, J = 2.7 Hz, 3H), 1.26-1.16 (m, 6H); |
| | | | ¹⁹F NMR (376 MHz, CDCl₃) δ −62.07 |
| J285 | | HRMS-ESI (m/z) | ¹H NMR (400 MHz, CDCl₃) δ 9.21 (s, 1H), 8.73 (s, 1H), 8.21 (d, |
| | | [M + H]⁺ calcd for | J = 8.6 Hz, 1H), 8.13 (d, J = 1.6 Hz, 2H), 8.11-7.93 (m, 2H), |
| | | C₃₀H₂₉F₃N₈O₂S, | 7.33 (d, J = 8.8 Hz, 1H), 7.13 (s, 1H), 6.89 (dd, J = 8.8, 2.8 Hz, |
| | | 623.2159; found, | 1H), 6.38 (d, J = 2.7 Hz, 1H), 3.97 (s, 2H), 2.97 (s, 6H), 2.61 (p, |
| | | 623.2165 | J = 6.9 Hz, 1H), 2.27 (s, 3H), 1.25-1.11 (m, 6H); ¹⁹F NMR |
| | | | (376 MHz, CDCl₃) δ −62.07 |
| J286 | | HRMS-ESI (m/z) | ¹H NMR (400 MHz, CDCl₃) δ 9.21 (d, J = 0.6 Hz, 1H), 8.73 (s, |
| | | [M + H]⁺ calcd for | 1H), 8.20 (dd, J = 8.5, 3.2 Hz, 1H), 8.17-8.10 (m, 2H), 8.10- |
| | | C₂₉H₂₆F₃N₇O₃S, | 7.94 (m, 2H), 7.54 (dd, J = 8.0, 3.2 Hz, 1H), 7.38 (t, J = 6.1 Hz, |
| | | 610.1843; found, | 1H), 7.04 (d, J = 22.3 Hz, 1H), 6.94 (s, 1H), 4.23-4.10 (m, |
| | | 610.1845 | 1H), 4.06-3.90 (m, 2H), 3.17 (d, J = 38.6 Hz, 3H), 2.43 (d, J = |
| | | | 2.0 Hz, 3H), 2.23 (d, J = 23.7 Hz, 3H), 1.38 (dd, J = 26.1, 6.4 |
| | | | Hz, 3H); ¹⁹F NMR (376 MHz, CDCl₃) δ −62.08 |
| J287 | | HRMS-ESI (m/z) | ¹H NMR (400 MHz, CDCl₃) δ 8.65 (s, 1H), 8.49 (t, J = 8.3 Hz, |
| | | [M + H]⁺ calcd for | 1H), 7.97 (dd, J = 8.7, 1.6 Hz, 1H), 7.93-7.89 (m, 3H), 7.86- |
| | | C₂₉H₂₄FN₇O₂S, | 7.80 (m, 2H), 7.54 (d, J = 3.0 Hz, 1H), 7.39 (d, J = 8.0 Hz, 1H), |
| | | 554.1769; found, | 7.33 (dd, J = 8.1, 1.8 Hz, 1H), 6.92-6.88 (m, 1H), 3.99 (d, J = |
| | | 554.1764 | 2.3 Hz, 2H), 2.71-2.63 (m, 1H), 2.17 (d, J = 2.4 Hz, 3H), 1.20 |
| | | | (dd, J = 8.8, 6.8 Hz, 6H); ¹⁹F NMR (376 MHz, CDCl₃) δ −130.84 |
| J288 | | HRMS-ESI (m/z) | ¹H NMR (400 MHz, CDCl₃) δ 8.69 (s, 1H), 8.50 (t, J = 8.4 Hz, |
| | | [M + H]⁺ calcd for | 1H), 7.99-7.89 (m, 4H), 7.88-7.82 (m, 2H), 7.56 (d, J = 3.1 |
| | | C₂₉H₂₁F₄N₇O₃S, | Hz, 1H), 7.32-7.25 (m, 1H), 7.04-7.01 (m, 1H), 6.97 (d, J = |
| | | 624.1435; found, | 8.5 Hz, 1H), 4.22 (tt, J = 7.0, 3.4 Hz, 2H), 3.94 (d, J = 1.2 Hz, |
| | | 624.1433 | 2H), 2.54 (dddd, J = 16.5, 10.5, 4.2, 1.6 Hz, 2H), 2.38 (s, 3H); |
| | | | ¹⁹F NMR (376 MHz, CDCl₃) δ −64.63, −131.25 |
| J289 | | ESIMS m/z | ¹H NMR (400 MHz, CDCl₃) δ 8.65 (s, 1H), 8.49 (t, J = 8.3 Hz, |
| | | 624 ([M + H]⁺) | 1H), 7.97 (dd, J = 8.6, 1.6 Hz, 1H), 7.94-7.90 (m, 2H), 7.90- |
| | | | 7.79 (m, 3H), 7.49 (d, J = 3.1 Hz, 1H), 7.42 (d, J = 7.8 Hz, 1H), |
| | | | 7.37-7.33 (m, 1H), 7.04 (d, J = 1.6 Hz, 1H), 4.63 (d, J = 12.4 |
| | | | Hz, 1H), 4.50 (d, J = 12.4 Hz, 1H), 3.96 (d, J = 2.7 Hz, 2H), |
| | | | 3.72 (qd, J = 8.9, 3.8 Hz, 2H), 2.45 (s, 3H); |
| | | | ¹⁹F NMR (376 MHz, CDCl₃) δ −73.78, −131.20 |
| J290 | | ESIMS m/z | ¹H NMR (400 MHz, CDCl₃) δ 8.65 (s, 1H), 8.50 (t, J = 8.3 Hz, |
| | | 583 ([M + H]⁺) | 1H), 7.99-7.95 (m, 1H), 7.93-7.81 (m, 5H), 7.60 (d, J = 3.0 |
| | | | Hz, 1H), 7.33 (d, J = 8.8 Hz, 1H), 6.89 (dd, J = 8.8, 2.7 Hz, 1H), |
| | | | 6.36 (d, J = 2.7 Hz, 1H), 3.99 (d, J = 1.7 Hz, 2H), 2.97 (s, 6H), |
| | | | 2.58 (p, J = 6.8 Hz, 1H), 1.17 (dd, J = 8.9, 6.8 Hz, 6H); ¹⁹F |
| | | | NMR (376 MHz, CDCl₃) δ −130.54 |
| J291 | | ESIMS m/z | ¹H NMR (400 MHz, CDCl₃) δ 8.65 (s, 1H), 8.52-8.44 (m, 1H), |
| | | 570 ([M + H]⁺) | 7.97 (dd, J = 8.6, 1.6 Hz, 1H), 7.94-7.81 (m, 5H), 7.53 (dd, J = |
| | | | 8.0, 4.2 Hz, 1H), 7.48-7.44 (m, 1H), 7.39 (t, J = 5.5 Hz, 1H), |
| | | | 6.93 (s, 1H), 4.21-4.11 (m, 1H), 4.00 (dd, J = 3.0, 1.9 Hz, |
| | | | 2H), 3.15 (d, J = 22.1 Hz, 3H), 2.43 (d, J = 1.0 Hz, 3H), 1.37 |
| | | | (dd, J = 15.8, 6.4 Hz, 3H); ¹⁹F NMR (376 MHz, CDCl₃) |
| | | | δ −130.92, −131.35 |
| J292 | | HRMS-ESI (m/z) | ¹H NMR (400 MHz, CDCl₃) δ 9.21 (s, 1H), 8.73 (d, J = 2.1 Hz, |
| | | [M + H]⁺ calcd for | 1H), 8.21 (d, J = 8.5 Hz, 1H), 8.13 (d, J = 1.6 Hz, 2H), 8.10- |
| | | C₂₉H₂₆F₃N₇O₃S, | 7.98 (m, 2H), 7.39 (d, J = 8.7 Hz, 1H), 7.14-7.03 (m, 2H), |
| | | 610.1843; found, | 6.63 (d, J = 2.7 Hz, 1H), 3.98 (d, J = 1.0 Hz, 2H), 3.83 (s, 3H), |
| | | 610.1845 | 2.65 (p, J = 6.8 Hz, 1H), 2.27 (s, 3H), 1.26-1.14 (m, 6H); ¹⁹F |
| | | | NMR (376 MHz, CDCl₃) δ −62.07 |
| J293 | | HRMS-ESI (m/z) | ¹H NMR (400 MHz, CDCl₃) δ 9.21 (s, 1H), 8.73 (d, J = 1.9 Hz, |
| | | [M + H]⁺ calcd for | 1H), 8.22 (d, J = 8.5 Hz, 1H), 8.13 (d, J = 1.6 Hz, 2H), 8.10- |
| | | C₂₉H₂₆F₃N₇O₂S, | 7.97 (m, 2H), 7.29 (t, J = 8.1 Hz, 1H), 7.10 (s, 1H), 6.94 (s, 1H), |
| | | 594.1894; found, | 3.97 (d, J = 1.8 Hz, 2H), 2.48-2.32 (m, 5H), 2.27 (s, 3H), 1.69- |
| | | 594.1894 | 1.54 (m, 3H), 0.93 (t, J = 7.3 Hz, 3H); |
| | | | ¹⁹F NMR (376 MHz, CDCl₃) δ −62.07 |
| J294 | | HRMS-ESI (m/z) | ¹H NMR (400 MHz, CDCl₃) δ 9.21 (s, 1H), 8.81-8.66 (m, 1H), |
| | | [M + H]⁺ calcd for | 8.22 (d, J = 8.5 Hz, 1H), 8.13 (d, J = 1.6 Hz, 2H), 8.08-7.96 |
| | | C₃₀H₂₉F₃N₈O₂S, | (m, 2H), 7.14 (s, 1H), 6.82 (dd, J = 8.6, 2.8 Hz, 1H), 6.42 (d, J = |
| | | 623.2159; found, | 2.7 Hz, 1H), 3.96 (d, J = 1.4 Hz, 2H), 2.97 (s, 6H), 2.33 (td, J = |
| | | 623.2169 | 7.5, 5.4 Hz, 2H), 2.28 (s, 3H), 1.57 (q, J = 7.3 Hz, 3H), 0.92 |
| | | | (t, J = 7.3 Hz, 3H); ¹⁹F NMR (376 MHz, CDCl₃) δ −62.07 |
| J295 | | | ¹H NMR (400 MHz, CDCl₃) δ 9.22 (s, 1H), 8.74 (dd, J = 2.2, 1.1 |
| | | | Hz, 1H), 8.53 (d, J = 8.7 Hz, 1H), 8.22 (d, J = 1.9 Hz, 1H), 8.19- |
| | | | 8.06 (m, 3H), 7.74 (s, 1H), 7.44-7.29 (m, 2H), 6.90 (s, 1H), |
| | | | 4.00 (d, J = 0.9 Hz, 2H), 2.67 (p, J = 6.8 Hz, 1H), 2.39 (s, 3H), |
| | | | 1.31-1.09 (m, 6H); ¹⁹F NMR (376 MHz, CDCl₃) δ −62.10 |
| J296 | | | ¹H NMR (400 MHz, CDCl₃) δ 9.22 (s, 1H), 8.74 (t, J = 1.3 Hz, |
| | | | 1H), 8.54 (d, J = 8.6 Hz, 1H), 8.19 (dd, J = 23.7, 1.9 Hz, 1H), |
| | | | 8.15-8.02 (m, 3H), 7.77 (s, 1H), 7.33 (d, J = 8.8 Hz, 1H), 6.89 |
| | | | (dd, J = 8.8, 2.8 Hz, 1H), 6.36 (d, J = 2.7 Hz, 1H), 3.99 (s, 2H), |
| | | | 2.97 (s, 6H), 2.59 (p, J = 6.8 Hz, 1H), 1.30-1.07 (m, 6H); ¹⁹F |
| | | | NMR (376 MHz, CDCl₃) δ −62.10 |
| J297 | | | ¹H NMR (400 MHz, CDCl₃) δ 9.21 (s, 1H), 8.73 (d, J = 2.1 Hz, |
| | | | 1H), 8.21 (d, J = 8.5 Hz, 1H), 8.13 (d, J = 1.5 Hz, 2H), 8.10- |
| | | | 7.95 (m, 2H), 7.49-7.29 (m, 2H), 7.05 (s, 2H), 4.72-4.40 (m, |
| | | | 2H), 3.94 (d, J = 2.1 Hz, 2H), 3.73 (q, J = 8.7 Hz, 2H), 2.45 (s, |
| | | | 3H), 2.27 (s, 3H); ¹⁹F NMR (376 MHz, CDCl₃) δ −62.08, −73.76 |
| J298 | | | ¹H NMR (400 MHz, CDCl₃) δ 9.22 (s, 1H), 8.74 (dt, J = 1.9, 0.9 |
| | | | Hz, 1H), 8.51 (t, J = 8.4 Hz, 1H), 8.20-8.07 (m, 2H), 8.07- |
| | | | 7.96 (m, 1H), 7.91 (dd, J = 12.0, 1.9 Hz, 1H), 7.51 (d, J = 2.7 |
| | | | Hz, 1H), 7.41 (d, J = 7.8 Hz, 1H), 7.38-7.30 (m, 1H), 7.02 (s, |
| | | | 1H), 4.44-4.23 (m, 2H), 3.96 (d, J = 4.0 Hz, 2H), 3.27 (s, 3H), |
| | | | 2.44 (s, 3H); ¹⁹F NMR (376 MHz, CDCl₃) δ −62.10, −131.10 |
| J299 | | | ¹H NMR (400 MHz, CDCl₃) δ 9.22 (s, 1H), 8.74 (dd, J = 2.2, 1.1 |
| | | | Hz, 1H), 8.49 (td, J = 8.4, 4.8 Hz, 1H), 8.20-8.06 (m, 2H), |
| | | | 8.00 (d, J = 8.6 Hz, 1H), 7.95-7.84 (m, 1H), 7.55-7.37 (m, |
| | | | 2H), 7.28 (s, 1H), 7.00 (dd, J = 8.0, 1.2 Hz, 1H), 4.13 (ddd, J = |
| | | | 14.3, 8.6, 6.7 Hz, 1H), 4.00 (dd, J = 3.0, 1.5 Hz, 2H), 3.17 (d, J = |
| | | | 19.8 Hz, 3H), 2.54-2.40 (m, 3H), 1.38 (dd, J = 14.3, 6.4 Hz, |
| | | | 3H); ¹⁹F NMR (376 MHz, CDCl₃) δ −62.10, −131.10 |
| J300 | | | ¹H NMR (400 MHz, CDCl₃) δ 9.21 (s, 1H), 8.74 (dt, J = 2.0, 1.0 |
| | | | Hz, 1H), 8.51 (t, J = 8.4 Hz, 1H), 8.20-8.05 (m, 2H), 7.99 (dd, |
| | | | J = 8.6, 1.5 Hz, 1H), 7.91 (dd, J = 12.0, 1.9 Hz, 1H), 7.51 (d, J = |
| | | | 2.8 Hz, 1H), 7.40 (d, J = 7.8 Hz, 1H), 7.37-7.28 (m, 1H), |
| | | | 7.01 (s, 1H), 4.51-4.27 (m, 2H), 3.95 (d, J = 0.9 Hz, 2H), 3.41 |
| | | | (q, J = 7.0 Hz, 2H), 2.43 (s, 3H), 1.15 (t, J = 7.0 Hz, 3H); ¹⁹F |
| | | | NMR (376 MHz, CDCl₃) δ −62.10, −131.07 |
| J301 | | HRMS-ESI (m/z) | ¹H NMR (400 MHz, CDCl₃) δ 9.24 (s, 1H), 8.76-8.73 (m, 1H), |
| | | [M + H]⁺ calcd for | 8.47 (d, J = 9.0 Hz, 1H), 8.43 (s, 1H), 8.37 (d, J = 8.7 Hz, 1H), |
| | | C₂₉H₂₃F₆N₇O₂S, | 8.17-8.10 (m, 2H), 7.54 (s, 1H), 7.21 (d, J = 7.9 Hz, 1H), 7.06 |
| | | 648.1611; found, | (dd, J = 7.8, 1.8 Hz, 1H), 6.91-6.85 (m, 1H), 4.00 (s, 2H), |
| | | 648.1611 | 3.03 (p, J = 6.9 Hz, 1H), 2.38 (s, 3H), 1.16 (dd, J = 6.9, 5.5 Hz, |
| | | | 6H); ¹⁹F NMR (376 MHz, CDCl₃) δ −60.76, −62.12, −121.08 |
| J302 | | HRMS-ESI (m/z) | ¹H NMR (500 MHz, CDCl₃) δ 9.24 (s, 1H), 8.75 (s, 1H), 8.50- |
| | | [M + H]⁺ calcd for | 8.42 (m, 2H), 8.38 (d, J = 8.9 Hz, 1H), 8.15 (d, J = 2.7 Hz, 2H), |
| | | C₃₀H₂₆F₆N₈O₂S, | 7.58 (s, 1H), 7.32 (d, J = 8.8 Hz, 1H), 6.93-6.86 (m, 1H), 6.35 |
| | | 677.1876; found, | (d, J = 2.7 Hz, 1H), 4.00 (s, 2H), 2.96 (s, 6H), 2.57 (q, J = 6.9 |
| | | 677.1879 | Hz, 1H), 1.17 (dd, J = 21.6, 6.8 Hz, 6H); ¹⁹F NMR (471 MHz, |
| | | | CDCl₃) δ −60.71, −62.10 |
| J303 | | HRMS-ESI (m/z) | ¹H NMR (400 MHz, CDCl₃) δ 9.24 (s, 1H), 8.76-8.73 (m, 1H), |
| | | [M + H]⁺ calcd for | 8.52-8.42 (m, 2H), 8.38 (d, J = 8.8 Hz, 1H), 8.25-8.11 (m, |
| | | C₂₉H₂₃F₆N₇O₃S, | 2H), 7.51 (dd, J = 15.9, 8.2 Hz, 2H), 7.38 (d, J = 8.8 Hz, 1H), |
| | | 664.156; | 6.94-6.89 (m, 1H), 4.21-4.08 (m, 1H), 4.00 (d, J = 2.2 Hz, |
| | | found, 664.1562 | 2H), 3.11 (d, J = 3.3 Hz, 3H), 2.42 (s, 3H), 1.44-1.32 (m, 3H); |
| | | | ¹⁹F NMR (376 MHz, CDCl₃) δ −60.77, −60.92, −62.12 |
| J304 | | HRMS-ESI (m/z) | ¹H NMR (400 MHz, CDCl₃) δ 9.24 (s, 1H), 8.75 (s, 1H), 8.48 (s, |
| | | [M + H]⁺ calcd for | 1H), 8.44 (s, 1H), 8.39 (s, 1H), 8.20-8.11 (m, 2H), 7.49 (s, |
| | | C₂₉H₂₀F₉N₇O₃S, | 1H), 7.40 (d, J = 7.7 Hz, 1H), 7.34 (d, J = 7.8 Hz, 1H), 7.04 (s, |
| | | 718.1277; found, | 1H), 4.67-4.47 (m, 2H), 3.96 (d, J = 2.2 Hz, 2H), 3.70 (dd, J = |
| | | 718.1279 | 8.7, 4.1 Hz, 1H), 2.45 (s, 3H); |
| | | | ¹⁹F NMR (376 MHz, CDCl₃) δ −60.73, −62.12, −73.83 |
| J305 | | HRMS-ESI (m/z) | ¹H NMR (400 MHz, CDCl₃) δ 9.24 (s, 1H), 8.75 (s, 1H), 8.51 (d, |
| | | [M + H]⁺ calcd for | J = 8.8 Hz, 1H), 8.44 (s, 1H), 8.37 (d, J = 8.4 Hz, 1H), 8.18- |
| | | C₂₉H₂₀F₉N₇O₃S, | 8.12 (m, 2H), 7.52 (s, 1H), 7.29 (s, 1H), 7.03 (s, 1H), 6.96 (d, J = |
| | | 718.1277; found, | 8.4 Hz, 1H), 4.22 (s, 2H), 3.94 (d, J = 1.4 Hz, 2H), 2.62- |
| | | 718.1275 | 2.46 (m, 2H), 2.38 (s, 3H); ¹⁹F NMR (376 MHz, CDCl₃) |
| | | | δ −60.73, −62.12, −64.69 |
| J306 | | | ¹H NMR (400 MHz, CDCl₃) δ 9.22 (d, J = 0.6 Hz, 1H), 8.80- |
| | | | 8.69 (m, 1H), 8.53 (d, J = 8.7 Hz, 1H), 8.21 (dd, J = 4.9, 1.9 Hz, |
| | | | 1H), 8.16-8.06 (m, 3H), 7.69 (d, J = 20.3 Hz, 1H), 7.54 (d, J = |
| | | | 8.0 Hz, 1H), 7.38 (d, J = 8.2 Hz, 1H), 6.93 (s, 1H), 4.16 (dq, J = |
| | | | 18.7, 6.4 Hz, 1H), 4.00 (d, J = 1.5 Hz, 2H), 3.17 (d, J = 40.7 |
| | | | Hz, 3H), 2.43 (s, 3H), 1.38 (dd, J = 30.6, 6.4 Hz, 3H); ¹⁹F NMR |
| | | | (376 MHz, CDCl₃) δ −62.10 |
| J307 | | | ¹H NMR (400 MHz, CDCl₃) δ 9.22 (s, 1H), 8.74 (dt, J = 2.0, 0.9 |
| | | | Hz, 1H), 8.52 (t, J = 8.4 Hz, 1H), 8.19-8.05 (m, 2H), 8.00 (dd, |
| | | | J = 8.5, 1.5 Hz, 1H), 7.91 (dd, J = 12.0, 1.9 Hz, 1H), 7.52 (d, J = |
| | | | 2.8 Hz, 1H), 7.34-7.27 (m, 1H), 7.07-7.01 (m, 1H), 6.97 |
| | | | (d, J = 8.4 Hz, 1H), 4.22 (tt, J = 6.9, 3.3 Hz, 2H), 3.94 (d, J = |
| | | | 1.7 Hz, 2H), 2.64-2.46 (m, 2H), 2.38 (s, 3H); ¹⁹F NMR (376 |
| | | | MHz, CDCl₃) δ −62.10, −64.60, −131.38 |
| J308 | | | ¹H NMR (400 MHz, CDCl₃) δ 8.68 (d, J = 2.2 Hz, 1H), 8.61 (d, |
| | | | J = 2.7 Hz, 1H), 8.44 (t, J = 8.5 Hz, 1H), 8.18 (d, J = 8.7 Hz, |
| | | | 1H), 8.04 (dd, J = 8.7, 2.3 Hz, 1H), 7.74-7.59 (m, 2H), 7.48- |
| | | | 7.38 (m, 2H), 7.38-7.29 (m, 1H), 7.04 (s, 1H), 6.77 (d, J = 2.7 |
| | | | Hz, 1H), 4.63 (d, J = 12.4 Hz, 1H), 4.50 (d, J = 12.4 Hz, 1H), |
| | | | 3.96 (d, J = 2.7 Hz, 2H), 3.72 (qd, J = 8.7, 3.9 Hz, 2H), 2.45 (s, |
| | | | 3H); ¹⁹F NMR (376 MHz, CDCl₃) δ −61.93, −73.79, −131.35 |
| J309 | | | ¹H NMR (400 MHz, CDCl₃) δ 8.67 (dt, J = 2.3, 0.9 Hz, 1H), |
| | | | 8.61 (d, J = 2.8 Hz, 1H), 8.42 (td, J = 8.5, 4.8 Hz, 1H), 8.18 (d, |
| | | | J = 9.0 Hz, 1H), 8.04 (dd, J = 8.8, 2.4 Hz, 1H), 7.72-7.60 (m, |
| | | | 2H), 7.53 (dd, J = 8.1, 3.9 Hz, 1H), 7.50-7.34 (m, 2H), 6.93 |
| | | | (s, 1H), 6.77 (dd, J = 2.7, 1.1 Hz, 1H), 4.25-4.08 (m, 1H), |
| | | | 4.08-3.90 (m, 2H), 3.16 (d, J = 22.8 Hz, 3H), 2.52-2.37 (m, |
| | | | 3H), 1.37 (dd, J = 17.1, 6.4 Hz, 3H); |
| | | | ¹⁹F NMR (376 MHz, CDCl₃) δ −61.93, −131.52 |
| J310 | | | ¹H NMR (400 MHz, CDCl₃) δ 8.67 (d, J = 2.1 Hz, 1H), 8.60 (d, |
| | | | J = 2.7 Hz, 1H), 8.29-8.17 (m, 1H), 8.11 (d, J = 9.1 Hz, 1H), |
| | | | 8.03 (dd, J = 8.7, 2.3 Hz, 1H), 7.74 (d, J = 7.2 Hz, 2H), 7.43 (d, |
| | | | J = 7.8 Hz, 1H), 7.34 (d, J = 7.8 Hz, 1H), 7.03 (d, J = 19.2 Hz, |
| | | | 2H), 6.78 (d, J = 2.7 Hz, 1H), 4.63 (d, J = 12.4 Hz, 1H), 4.52 (d, |
| | | | J = 12.4 Hz, 1H), 3.94 (d, J = 2.1 Hz, 2H), 3.73 (q, J = 8.7 Hz, |
| | | | 2H), 2.45 (s, 3H), 2.26 (s, 3H); |
| | | | ¹⁹F NMR (376 MHz, CDCl₃) δ −61.90, −73.76 |
| J311 | | | ¹H NMR (400 MHz, CDCl₃) δ 8.72-8.62 (m, 1H), 8.60 (d, J = |
| | | | 2.7 Hz, 1H), 8.19 (d, J = 8.6 Hz, 1H), 8.11 (dd, J = 9.1, 3.8 Hz, |
| | | | 1H), 8.03 (dd, J = 8.8, 2.4 Hz, 1H), 7.74 (d, J = 6.7 Hz, 2H), |
| | | | 7.54 (dd, J = 8.0, 3.1 Hz, 1H), 7.38 (t, J = 6.3 Hz, 1H), 7.05- |
| | | | 6.88 (m, 2H), 6.78 (dd, J = 2.8, 1.5 Hz, 1H), 4.26-4.09 (m, |
| | | | 1H), 4.04-3.89 (m, 2H), 3.17 (d, J = 39.4 Hz, 3H), 2.43 (d, J = |
| | | | 1.9 Hz, 3H), 2.22 (d, J = 23.5 Hz, 3H), 1.38 (dd, J = 27.3, 6.4 |
| | | | Hz, 3H); ¹⁹F NMR (376 MHz, CDCl₃) δ −61.90 |
| J312 | | ESIMS m/z 629 | ¹H NMR (400 MHz, CDCl₃) δ 8.54 (s, 1H), 8.48 (t, J = 8.4 Hz, |
| | | ([M + H]⁺) | 1H), 7.97 (d, J = 9.3 Hz, 1H), 7.89 (dd, J = 11.9, 1.9 Hz, 1H), |
| | | | 7.83-7.73 (m, 2H), 7.56 (d, J = 3.0 Hz, 1H), 7.45-7.33 (m, |
| | | | 3H), 7.08 (dd, J = 8.7, 2.7 Hz, 1H), 6.61 (d, J = 2.7 Hz, 1H), |
| | | | 4.00 (d, J = 2.1 Hz, 2H), 3.83 (s, 3H), 2.63 (p, J = 6.8 Hz, 1H), |
| | | | 1.23-1.11 (m, 6H); |
| | | | ¹⁹F NMR (376 MHz, CDCl₃) δ −57.67-−58.15 (m), −131.00 |
| J313 | | HRMS-ESI (m/z) | ¹H NMR (300 MHz, CDCl₃) δ 8.54 (d, J = 0.4 Hz, 1H), 8.18 (d, |
| | | [M + H]⁺ calcd for | J = 8.4 Hz, 1H), 8.12 (d, J = 8.7 Hz, 2H), 7.83-7.75 (m, 2H), |
| | | C₂₆H₁₈F₃N₇O₅S, | 7.59 (d, J = 8.7 Hz, 2H), 7.50-7.45 (m, 1H), 7.38 (d, J = 8.6 |
| | | 598.1115; found, | Hz, 2H), 7.25-7.21 (m, 2H), 4.13-3.92 (m, 2H), 2.54 (s, 3H) |
| | | 598.1122 | |
| J314 | | HRMS-ESI (m/z) | ¹H NMR (400 MHz, CDCl₃) δ 8.52 (d, J = 15.7 Hz, 2H), 8.25- |
| | | [M + H]⁺ calcd for | 8.02 (m, 2H), 7.87-7.69 (m, 3H), 7.45-7.29 (m, 4H), 6.90 (t, |
| | | C₂₉H₂₄ClF₃N₆O₃S, | J = 1.2 Hz, 1H), 3.99 (s, 1H), 2.67 (p, J = 6.9 Hz, 1H), 2.39 (s, |
| | | 629.1344; found, | 3H), 1.20 (dd, J = 20.2, 6.9 Hz, 6H); |
| | | 629.1341 | ¹⁹F NMR (376 MHz, CDCl₃) δ −58.03 |
| J315 | | HRMS-ESI (m/z) | ¹H NMR (400 MHz, CDCl₃) δ 8.62 (s, 1H), 8.20-8.08 (m, 2H), |
| | | [M + H]⁺ calcd for | 7.90 (d, J = 8.5 Hz, 2H), 7.79 (d, J = 8.5 Hz, 2H), 7.67-7.55 |
| | | C₂₉H₂₂F₆N₆O₃S, | (m, 2H), 7.33 (d, J = 26.9 Hz, 2H), 7.08-7.01 (m, 1H), 6.96 (d, |
| | | 649.1451; found, | J = 8.4 Hz, 1H), 4.21 (td, J = 6.3, 1.8 Hz, 2H), 3.92 (d, J = 1.9 |
| | | 649.1452 | Hz, 2H), 2.63-2.47 (m, 2H), 2.37 (s, 3H); |
| | | | ¹⁹F NMR (376 MHz, CDCl₃) δ −62.49, −64.59 |
| J316 | | HRMS-ESI (m/z) | ¹H NMR (400 MHz, CDCl₃) δ 8.53 (s, 1H), 8.17-8.07 (m, 2H), |
| | | [M + H]⁺ calcd for | 7.86-7.71 (m, 2H), 7.66-7.57 (m, 2H), 7.44-7.28 (m, 3H), |
| | | C₂₉H₂₂F₆N₆O₄S, | 7.04 (d, J = 2.1 Hz, 1H), 6.96 (d, J = 8.4 Hz, 1H), 4.21 (td, J = |
| | | 665.14; found, | 6.3, 1.8 Hz, 2H), 3.92 (d, J = 1.9 Hz, 2H), 2.54 (dtd, J = 14.7, |
| | | 665.1406 | 6.5, 3.4 Hz, 2H), 2.37 (s, 3H); |
| | | | ¹⁹F NMR (376 MHz, CDCl₃) δ −58.03, −64.60 |
| J317 | | HRMS-ESI (m/z) | ¹H NMR (400 MHz, CDCl₃) δ 8.64 (s, 1H), 8.52 (d, J = 8.7 Hz, |
| | | [M + H]⁺ calcd for | 1H), 8.21 (s, 1H), 8.17-8.04 (m, 1H), 7.90 (d, J = 8.4 Hz, 2H), |
| | | C₂₉H₂₁ClF₆N₆O₃S, | 7.80 (d, J = 8.6 Hz, 2H), 7.31 (d, J = 8.2 Hz, 1H), 7.08 (d, J = |
| | | 683.1061; found, | 19.6 Hz, 2H), 7.04-6.92 (m, 1H), 5.76 (d, J = 1.6 Hz, 1H), |
| | | 683.1044 | 4.34-4.14 (m, 3H), 2.54 (s, 3H), 2.39 (d, J = 5.2 Hz, 2H); |
| | | | ¹⁹F NMR (376 MHz, CDCl₃) δ −62.52, −64.68 |
| J318 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.96 (dd, J = 8.5, 1.6 Hz, 1H), 7.88 (dd, J = 11.9, 1.9 Hz, |
| | | C₃₀H₂₇F₄N₇O₃S, | 1H), 7.83-7.74 (m, 3H), 7.57 (d, J = 2.9 Hz, 1H), 7.44-7.30 |
| | | 642.1905; found, | (m, 2H), 6.87 (ddd, J = 19.0, 8.8, 2.8 Hz, 1H), 6.36 (d, J = 2.7 |
| | | 642.1884 | Hz, 1H), 3.99 (d, J = 1.7 Hz, 2H), 2.95 (d, J = 9.5 Hz, 6H), 2.58 |
| | | | (h, J = 6.7 Hz, 1H), 1.16 (m, 6H); |
| | | | ¹⁹F NMR (376 MHz, CDCl₃) δ −58.03, −130.74 |
| J319 | | ESIMS m/z 624 | ¹H NMR (400 MHz, CDCl₃) δ 8.53 (s, 1H), 8.20-8.07 (m, 2H), |
| | | ([M + H]⁺) | 7.85-7.74 (m, 2H), 7.70-7.58 (m, 2H), 7.44 (s, 1H), 7.42- |
| | | | 7.29 (m, 3H), 6.89 (dd, J = 8.8, 2.8 Hz, 1H), 6.37 (d, J = 2.7 Hz, |
| | | | 1H), 3.98 (d, J = 2.4 Hz, 2H), 2.96 (s, 6H), 2.60 (hept, J = 6.9 |
| | | | Hz, 1H), 1.17 (dd, J = 6.9, 5.6 Hz, 6H); |
| | | | ¹⁹F NMR (376 MHz, CDCl₃) δ −58.03 |
| J320 | | HRMS-ESI (m/z) | ¹H NMR (400 MHz, CDCl₃) δ 8.53 (s, 1H), 8.19-8.08 (m, 2H), |
| | | [M + H]⁺ calcd for | 7.86-7.71 (m, 2H), 7.69-7.54 (m, 2H), 7.44-7.30 (m, 3H), |
| | | C₂₉H₂₂F₆N₆O₅S, | 7.08-6.96 (m, 2H), 6.80 (dd, J = 2.4, 1.0 Hz, 1H), 4.18 (td, J = |
| | | 681.1349; found, | 6.2, 2.1 Hz, 2H), 3.93 (d, J = 2.0 Hz, 2H), 3.82 (s, 3H), 2.63- |
| | | 681.1358 | 2.43 (m, 2H); |
| | | | ¹⁹F NMR (376 MHz, CDCl₃) δ −58.03, −64.58 |
| J321 | | HRMS-ESI (m/z) | ¹H NMR (400 MHz, CDCl₃) δ 8.53 (s, 1H), 8.18-8.06 (m, 2H), |
| | | [M + H]⁺ calcd for | 7.86-7.73 (m, 2H), 7.68-7.59 (m, 2H), 7.46-7.33 (m, 3H), |
| | | C₃₀H₂₅F₆N₇O₄S, | 7.00 (d, J = 9.1 Hz, 1H), 6.83 (dd, J = 9.1, 3.1 Hz, 1H), 6.57 (d, |
| | | 694.1666; found, | J = 3.1 Hz, 1H), 4.25-4.11 (m, 2H), 3.93 (d, J = 2.2 Hz, 2H), |
| | | 694.1679 | 2.94 (s, 6H), 2.51 (qdd, J = 10.5, 5.2, 3.2 Hz, 2H); |
| | | | ¹⁹F NMR (376 MHz, CDCl₃) δ −58.03, −64.58 |
| J322 | | HRMS-ESI (m/z) | ¹H NMR (300 MHz, DMSO-d₆) δ 10.00 (s, 1H), 9.36 (s, 1H), |
| | | [M + H]⁺ calcd for | 8.76 (s, 1H), 8.09-8.03 (m, 2H), 7.99 (d, J = 8.7 Hz, 2H), 7.79 |
| | | C₂₈H₂₄F₃N₇O₃S, | (d, J = 8.8 Hz, 2H), 7.62 (d, J = 8.6 Hz, 4H), 7.44 (d, J = 8.3 |
| | | 596.1686; found, | Hz, 1H), 7.26 (d, J = 8.5 Hz, 1H), 7.06 (s, 1H), 4.16-3.99 (m, |
| | | 596.1697 | 2H), 2.77 (s, 3H), 2.31 (s, 3H) |
| J323 | 244-250 | HRMS-ESI (m/z) | ¹H NMR (500 MHz, CDCl₃) δ 8.61 (s, 1H), 8.18-8.11 (m, 2H), |
| | | [M + H]⁺ calcd for | 8.07-8.02 (m, 1H), 7.98-7.91 (m, 1H), 7.70-7.58 (m, 4H), |
| | | C₂₉H₂₅F₃N₆O₂S, | 7.42-7.36 (m, 2H), 7.33 (dd, J = 7.9, 1.8 Hz, 1H), 6.92 (dd, J = |
| | | 579.1785; found, | 2.0, 0.9 Hz, 1H), 3.98 (d, J = 4.6 Hz, 2H), 2.68 (p, J = 6.9 Hz, |
| | | 579.1788 | 1H), 2.39 (s, 3H), 1.20 (dd, J = 8.7, 6.9 Hz, 6H); |
| | | | ¹⁹F NMR (471 MHz, CDCl₃) δ −62.81 |
| J324 | 244-248 | HRMS-ESI (m/z) | ¹H NMR (500 MHz, CDCl₃) δ 8.61 (s, 1H), 8.18-8.11 (m, 2H), |
| | | [M + H]⁺ calcd for | 8.07-8.02 (m, 1H), 7.94 (m, 1H), 7.70-7.59 (m, 4H), 7.43- |
| | | C₂₉H₂₅F₃N₆O₃S, | 7.36 (m, 2H), 7.08 (dd, J = 8.8, 2.7 Hz, 1H), 6.63 (d, J = 2.7 Hz, |
| | | 595.1734; found, | 1H), 3.99 (d, J = 4.1 Hz, 2H), 3.82 (s, 3H), 2.65 (p, J = 6.8 Hz, |
| | | 595.1736 | 1H), 1.19 (dd, J = 6.8, 3.9 Hz, 6H); |
| | | | ¹⁹F NMR (471 MHz, CDCl₃) δ −62.81 |
| J325 | 182-187 | HRMS-ESI (m/z) | ¹H NMR (500 MHz, CDCl₃) δ 8.61 (s, 1H), 8.18-8.11 (m, 2H), |
| | | [M + H]⁺ calcd for | 8.07-8.02 (m, 1H), 7.94 (ddd, J = 5.8, 3.6, 2.3 Hz, 1H), 7.70- |
| | | C₃₀H₂₈F₃N₇O₂S, | 7.59 (m, 4H), 7.46 (s, 1H), 7.34 (d, J = 8.8 Hz, 1H), 6.90 (dd, J = |
| | | 608.2050; found, | 8.8, 2.7 Hz, 1H), 6.37 (d, J = 2.7 Hz, 1H), 3.98 (d, J = 3.7 Hz, |
| | | 608.2053 | 2H), 2.96 (s, 6H), 2.60 (p, J = 6.8 Hz, 1H), 1.17 (t, J = 7.1 Hz, |
| | | | 6H); |
| | | | ¹⁹F NMR (471 MHz, CDCl₃) δ −62.81 |
| J326 | 127-137 | HRMS-ESI (m/z) | ¹H NMR (500 MHz, CDCl₃) δ 8.61 (s, 1H), 8.18-8.11 (m, 2H), |
| | | [M + H]⁺ calcd for | 8.07-8.02 (m, 1H), 7.98-7.91 (m, 1H), 7.70-7.58 (m, 4H), |
| | | C₂₉H₂₂F₆N₆O₃S, | 7.34 (s, 1H), 7.28 (dd, J = 8.5, 2.0 Hz, 1H), 7.06-7.01 (m, |
| | | 649.1451; found, | 1H), 6.97 (d, J = 8.5 Hz, 1H), 4.22 (tt, J = 6.3, 3.4 Hz, 2H), 3.92 |
| | | 649.1451 | (d, J = 2.7 Hz, 2H), 2.61-2.47 (m, 2H), 2.38 (s, 3H); |
| | | | ¹⁹F NMR (471 MHz, CDCl₃) δ −62.80, −64.59 |
| J327 | 204-208 | HRMS-ESI (m/z) | ¹H NMR (500 MHz, CDCl₃) δ 8.57 (s, 1H), 8.17-8.10 (m, 2H), |
| | | [M + H]⁺ calcd for | 7.71-7.64 (m, 2H), 7.65-7.58 (m, 2H), 7.55 (t, J = 8.5 Hz, |
| | | C₂₉H₂₅F₃N₆O₃S, | 1H), 7.39 (d, J = 8.1 Hz, 2H), 7.33 (dd, J = 8.1, 1.8 Hz, 1H), |
| | | 595.1734; found, | 7.27-7.22 (m, 1H), 6.92 (t, J = 1.3 Hz, 1H), 3.98 (d, J = 4.6 |
| | | 595.1738 | Hz, 2H), 2.68 (p, J = 6.8 Hz, 1H), 2.39 (s, 3H), 1.20 (dd, J = |
| | | | 8.6, 6.9 Hz, 6H); |
| | | | ¹⁹F NMR (471 MHz, CDCl₃) δ −57.79 |
| J328 | 252-254 | HRMS-ESI (m/z) | ¹H NMR (500 MHz, CDCl₃) δ 8.57 (s, 1H), 8.17-8.10 (m, 2H), |
| | | [M + H]⁺ calcd for | 7.67 (ddd, J = 5.3, 2.4, 1.0 Hz, 2H), 7.66-7.58 (m, 2H), 7.55 |
| | | C₂₉H₂₅F₃N₆O₄S, | (t, J = 8.5 Hz, 1H), 7.43-7.37 (m, 2H), 7.27-7.22 (m, 1H), |
| | | 611.1683; found, | 7.07 (dd, J = 8.7, 2.7 Hz, 1H), 6.63 (d, J = 2.7 Hz, 1H), 3.99 (d, |
| | | 611.1685 | J = 4.2 Hz, 2H), 3.82 (s, 3H), 2.65 (p, J = 6.9 Hz, 1H), 1.19 (dd, |
| | | | J = 6.9, 3.9 Hz, 6H); |
| | | | ¹⁹F NMR (471 MHz, CDCl₃) δ −57.79 |
| J329 | 173-178 | HRMS-ESI (m/z) | ¹H NMR (500 MHz, CDCl₃) δ 8.57 (s, 1H), 8.17-8.10 (m, 2H), |
| | | [M + H]⁺ calcd for | 7.71-7.64 (m, 2H), 7.66-7.59 (m, 2H), 7.55 (t, J = 8.5 Hz, |
| | | C₃₀H₂₈F₃N₇O₃S, | 1H), 7.47 (s, 1H), 7.34 (d, J = 8.8 Hz, 1H), 7.25 (dd, J = 2.2, |
| | | 624.1999; found, | 1.1 Hz, 1H), 6.90 (dd, J = 8.9, 2.7 Hz, 1H), 6.37 (d, J = 2.7 Hz, |
| | | 624.2002 | 1H), 3.98 (d, J = 3.7 Hz, 2H), 2.96 (s, 6H), 2.60 (p, J = 6.8 Hz, |
| | | | 1H), 1.17 (t, J = 7.0 Hz, 6H); |
| | | | ¹⁹F NMR (471 MHz, CDCl₃) δ −57.78 |
| J330 | 222-225 | HRMS-ESI (m/z) | ¹H NMR (500 MHz, CDCl₃) δ 8.57 (s, 1H), 8.16-8.10 (m, 2H), |
| | | [M + H]⁺ calcd for | 7.71-7.64 (m, 2H), 7.62 (d, J = 8.7 Hz, 2H), 7.55 (t, J = 8.5 |
| | | C₂₉H₂₂F₆N₆O₄S, | Hz, 1H), 7.33 (s, 1H), 7.29 (d, J = 6.7 Hz, 2H), 7.04 (d, J = 2.2 |
| | | 665.1400; found, | Hz, 1H), 6.97 (d, J = 8.4 Hz, 1H), 4.22 (tt, J = 6.4, 3.4 Hz, 2H), |
| | | 665.1397 | 3.92 (d, J = 2.7 Hz, 2H), 2.54 (qd, J = 10.3, 4.1 Hz, 2H), 2.38 |
| | | | (s, 3H); |
| | | | ¹⁹F NMR (471 MHz, CDCl₃) δ −57.78, −64.59 |
| J331 | | HRMS-ESI (m/z) | ¹H NMR (400 MHz, CDCl₃) δ 8.53 (s, 1H), 8.18-8.07 (m, 2H), |
| | | [M + H]⁺ calcd for | 7.86-7.72 (m, 2H), 7.66-7.55 (m, 2H), 7.47-7.28 (m, 5H), |
| | | C₂₉H₂₂F₆N₆O₄S, | 7.11-7.00 (m, 1H), 4.62 (d, J = 12.3 Hz, 1H), 4.50 (d, J = 12.4 |
| | | 665.14; found, | Hz, 1H), 4.04-3.85 (m, 2H), 3.73 (qd, J = 8.7, 1.8 Hz, 2H), |
| | | 665.1409 | 2.44 (s, 3H); |
| | | | ¹⁹F NMR (376 MHz, CDCl₃) δ −58.02, −73.76 |
| J332 | | HRMS-ESI (m/z) | ¹H NMR (400 MHz, CDCl₃) δ 8.63 (s, 1H), 8.21-8.09 (m, 2H), |
| | | [M + H]⁺ calcd for | 7.90 (d, J = 8.4 Hz, 2H), 7.79 (d, J = 8.5 Hz, 2H), 7.66-7.58 |
| | | C₂₉H₂₂F₆N₆O₃S, | (m, 2H), 7.43 (d, J = 7.8 Hz, 1H), 7.38-7.29 (m, 2H), 7.11- |
| | | 649.1451; found, | 6.96 (m, 1H), 4.62 (d, J = 12.3 Hz, 1H), 4.50 (d, J = 12.4 Hz, |
| | | 649.1455 | 1H), 4.04-3.87 (m, 2H), 3.73 (qd, J = 8.7, 1.7 Hz, 2H), 2.44 |
| | | | (s, 3H); |
| | | | ¹⁹F NMR (376 MHz, CDCl₃) δ −62.48, −73.76 |
| J333 | | HRMS-ESI (m/z) | ¹H NMR (400 MHz, CDCl₃) δ 8.61 (s, 1H), 8.50 (t, J = 8.4 Hz, |
| | | [M + H]⁺ calcd for | 1H), 8.02-7.92 (m, 1H), 7.89 (dd, J = 12.0, 1.8 Hz, 1H), 7.83 |
| | | C₂₉H₂₁F₇N₆O₃S2, | (d, J = 1.2 Hz, 3H), 7.52 (d, J = 3.1 Hz, 1H), 7.33-7.27 (m, |
| | | 699.1078; found, | 1H), 7.07-6.91 (m, 2H), 4.22 (tt, J = 6.9, 3.3 Hz, 2H), 3.94 (d, |
| | | 699.1076 | J = 1.7 Hz, 2H), 2.54 (qdd, J = 10.7, 5.3, 3.2 Hz, 2H), 2.38 (s, |
| | | | 3H); |
| | | | ¹⁹F NMR (376 MHz, CDCl₃) δ −42.63, −64.59, −131.41 |
| J334 | | HRMS-ESI (m/z) | ¹H NMR (400 MHz, CDCl₃) δ 8.61 (s, 1H), 8.50 (t, J = 8.3 Hz, |
| | | [M + H]⁺ calcd for | 1H), 7.97 (d, J = 8.4 Hz, 1H), 7.89 (dd, J = 12.1, 1.8 Hz, 1H), |
| | | C₃₀H₂₇F₄N₇O₂S2, | 7.83 (d, J = 0.9 Hz, 3H), 7.58 (d, J = 3.0 Hz, 1H), 7.33 (d, J = |
| | | 658.1677; found, | 8.8 Hz, 1H), 6.89 (dd, J = 8.8, 2.7 Hz, 1H), 6.36 (d, J = 2.7 Hz, |
| | | 658.1679 | 1H), 3.99 (d, J = 1.7 Hz, 2H), 2.97 (s, 6H), 2.58 (p, J = 6.9 Hz, |
| | | | 2H), 1.17 (dd, J = 9.0, 6.8 Hz, 6H); |
| | | | ¹⁹F NMR (376 MHz, CDCl₃) δ −42.63, −130.66 |
| J335 | | HRMS-ESI (m/z) | ¹H NMR (400 MHz, CDCl₃) δ 8.63 (d, J = 0.7 Hz, 1H), 8.52 (d, |
| | | [M + H]⁺ calcd for | J = 8.6 Hz, 1H), 8.26-8.15 (m, 1H), 8.10 (d, J = 8.9 Hz, 1H), |
| | | C₂₉H₂₄ClF₃N₆O₃S, | 7.90 (d, J = 8.5 Hz, 2H), 7.80 (d, J = 8.5 Hz, 2H), 7.68 (d, J = |
| | | 629.1344; found, | 20.2 Hz, 1H), 7.54 (d, J = 8.0 Hz, 1H), 7.38 (d, J = 8.1 Hz, 1H), |
| | | 629.1339 | 6.93 (s, 1H), 4.16 (dt, J = 18.7, 6.5 Hz, 1H), 4.00 (d, J = 1.4 Hz, |
| | | | 2H), 3.22 (s, 2H), 3.12 (s, 1H), 2.43 (s, 3H), 1.38 (dd, J = 30.6, |
| | | | 6.4 Hz, 3H); |
| | | | ¹⁹F NMR (376 MHz, CDCl₃) δ −62.52 |
| J336 | | HRMS-ESI (m/z) | ¹H NMR (400 MHz, CDCl₃) δ 9.22 (s, 1H), 8.74 (dt, J = 1.9, 0.9 |
| | | [M + H]⁺ calcd for | Hz, 1H), 8.51 (t, J = 8.3 Hz, 1H), 8.19-8.06 (m, 2H), 8.00 (d, J = |
| | | C₂₉H₂₆F₄N₈O₂S, | 8.6 Hz, 1H), 7.91 (dd, J = 12.0, 1.9 Hz, 1H), 7.57 (d, J = 3.0 |
| | | 627.1908; found, | Hz, 1H), 7.33 (d, J = 8.8 Hz, 1H), 6.89 (dd, J = 8.8, 2.8 Hz, 1H), |
| | | 627.1921 | 6.36 (d, J = 2.7 Hz, 1H), 3.99 (d, J = 1.7 Hz, 2H), 2.97 (s, 6H), |
| | | | 2.59 (p, J = 6.8 Hz, 1H), 1.17 (dd, J = 9.3, 6.8 Hz, 6H); |
| | | | ¹⁹F NMR (376 MHz, CDCl₃) δ −62.10, −130.64 |
| J337 | | HRMS-ESI (m/z) | ¹H NMR (400 MHz, CDCl₃) δ 9.21 (s, 1H), 8.73 (dt, J = 2.0, 1.1 |
| | | [M + H]⁺ calcd for | Hz, 1H), 8.23-8.06 (m, 4H), 7.68-7.56 (m, 2H), 7.45-7.31 |
| | | C₂₈H₂₄F₃N₇O₂S, | (m, 3H), 6.92 (d, J = 1.8 Hz, 1H), 3.98 (d, J = 3.1 Hz, 2H), 2.68 |
| | | 580.1737; found, | (p, J = 6.8 Hz, 1H), 2.39 (s, 3H), 1.20 (t, J = 6.9 Hz, 6H); |
| | | 580.1748 | ¹⁹F NMR (376 MHz, CDCl₃) δ −62.08 |
| J338 | | HRMS-ESI (m/z) | ¹H NMR (400 MHz, CDCl₃) δ 9.21 (s, 1H), 8.73 (dt, J = 2.0, 1.0 |
| | | [M + H]⁺ calcd for | Hz, 1H), 8.19-8.08 (m, 4H), 7.58 (ddd, J = 30.4, 8.3, 5.7 Hz, |
| | | C₂₈H₂₄F₃N₇O₃S, | 3H), 7.37 (dd, J = 15.8, 8.7 Hz, 2H), 6.94 (s, 1H), 4.27-4.09 |
| | | 596.1686; found, | (m, 1H), 4.07-3.90 (m, 2H), 3.16 (d, J = 20.3 Hz, 3H), 2.52- |
| | | 596.169 | 2.36 (m, 3H), 1.37 (dd, J = 11.2, 6.4 Hz, 3H); |
| | | | ¹⁹F NMR (376 MHz, CDCl₃) δ −62.08 |
| J339 | | HRMS-ESI (m/z) | ¹H NMR (400 MHz, CDCl₃) δ 9.21 (s, 1H), 8.73 (dt, J = 2.0, 1.0 |
| | | [M + H]⁺ calcd for | Hz, 1H), 8.25-8.07 (m, 4H), 7.71-7.55 (m, 2H), 7.44 (s, 1H), |
| | | C₂₉H₂₇F₃N₈O₂S, | 7.34 (d, J = 8.8 Hz, 1H), 6.90 (dd, J = 8.8, 2.8 Hz, 1H), 6.37 (d, |
| | | 609.2003; found, | J = 2.7 Hz, 1H), 3.98 (d, J = 2.5 Hz, 2H), 2.96 (s, 6H), 2.60 (p, |
| | | 609.2014 | J = 6.9 Hz, 1H), 1.17 (dd, J = 6.8, 5.9 Hz, 6H); |
| | | | ¹⁹F NMR (376 MHz, CDCl₃) δ −62.08 |
| J340 | | | ¹H NMR (400 MHz, CDCl₃) δ 8.49 (s, 1H), 8.45 (dt, J = 8.4, 4.2 |
| | | | Hz, 1H), 7.99-7.95 (m, 1H), 7.88 (ddd, J = 12.0, 3.5, 1.9 Hz, |
| | | | 1H), 7.74-7.69 (m, 2H), 7.53 (dd, J = 8.0, 3.7 Hz, 1H), 7.46 |
| | | | (dd, J = 29.2, 3.0 Hz, 1H), 7.38 (d, J = 7.7 Hz, 1H), 7.16-7.10 |
| | | | (m, 2H), 6.93 (s, 1H), 4.84 (s, 2H), 4.20-4.10 (m, 1H), 3.99 |
| | | | (dd, J = 2.9, 1.8 Hz, 2H), 3.15 (d, J = 22.1 Hz, 3H), 2.43 (s, |
| | | | 3H), 1.37 (dd, J = 16.5, 6.4 Hz, 3H); |
| | | | ¹⁹F NMR (376 MHz, CDCl₃) δ −131.17, −131.61 |
| J341 | | HRMS-ESI (m/z) | ¹H NMR (400 MHz, CDCl₃) δ 9.20 (s, 1H), 8.60 (s, 1H), 8.48 |
| | | [M + H]⁺ calcd for | (td, J = 8.4, 4.8 Hz, 1H), 8.13-8.04 (m, 2H), 8.03-7.83 (m, |
| | | C₂₈H₂₄F₃N₇O₃S, | 2H), 7.53 (dt, J = 9.8, 4.9 Hz, 1H), 7.41 (dd, J = 19.2, 5.5 Hz, |
| | | 596.1686; found, | 2H), 6.92 (d, J = 5.8 Hz, 1H), 6.71 (d, J = 55.7 Hz, 1H), 4.19- |
| | | 596.169 | 4.09 (m, 1H), 4.10-3.88 (m, 2H), 3.16 (d, J = 22.5 Hz, 3H), |
| | | | 2.43 (s, 3H), 1.37 (dd, J = 16.6, 6.4 Hz, 3H); |
| | | | ¹⁹F NMR (376 MHz, CDCl₃) δ −112.03, −131.50 |
| J342 | | HRMS-ESI (m/z) | ¹H NMR (400 MHz, CDCl₃) δ 8.48 (s, 1H), 8.48 (t, J = 8.3 Hz, |
| | | [M + H]⁺ calcd for | 1H), 7.96 (dd, J = 8.6, 1.5 Hz, 1H), 7.88 (dd, J = 12.0, 1.8 Hz, |
| | | C₃₁H₂₉FN₈O₃S, | 1H), 7.75-7.69 (m, 2H), 7.56 (d, J = 3.0 Hz, 1H), 7.33 (d, J = |
| | | 613.214; found, | 8.8 Hz, 1H), 7.16-7.11 (m, 2H), 6.89 (dd, J = 8.8, 2.8 Hz, 1H), |
| | | 613.2151 | 6.36 (d, J = 2.7 Hz, 1H), 4.84 (s, 2H), 3.99 (d, J = 1.7 Hz, 2H), |
| | | | 2.97 (s, 6H), 2.58 (hept, J = 6.7 Hz, 1H), 1.17 (dd, J = 9.1, 6.8 |
| | | | Hz, 6H); |
| | | | ¹⁹F NMR (376 MHz, CDCl₃) δ −130.79 |
| J343 | | HRMS-ESI (m/z) | ¹H NMR (400 MHz, CDCl₃) δ 8.49 (s, 1H), 8.47 (t, J = 8.4 Hz, |
| | | [M + H]⁺ calcd for | 1H), 7.97 (d, J = 8.7 Hz, 1H), 7.88 (dd, J = 12.1, 1.8 Hz, 1H), |
| | | C₃₀H₂₃F₄N₇O₄S, | 7.75-7.69 (m, 2H), 7.47 (d, J = 3.1 Hz, 1H), 7.42 (d, J = 7.8 |
| | | 654.1541; found, | Hz, 1H), 7.35 (d, J = 7.8 Hz, 1H), 7.17-7.10 (m, 2H), 7.04 (s, |
| | | 654.1542 | 1H), 4.84 (s, 2H), 4.63 (d, J = 12.5 Hz, 1H), 4.50 (d, J = 12.4 |
| | | | Hz, 1H), 3.96 (d, J = 2.7 Hz, 2H), 3.71 (qd, J = 8.7, 4.3 Hz, 2H), |
| | | | 2.45 (s, 3H); |
| | | | ¹⁹F NMR (376 MHz, CDCl₃) δ −73.78, −131.46 |
| J344 | | HRMS-ESI (m/z) | ¹H NMR (400 MHz, CDCl₃) δ 8.49 (s, 1H), 8.47 (t, J = 8.4 Hz, |
| | | [M + H]⁺ calcd for | 1H), 7.96 (d, J = 8.6 Hz, 1H), 7.88 (d, J = 11.9 Hz, 1H), 7.72 (d, |
| | | C₃₀H₂₆FN₇O₃S, | J = 9.0 Hz, 2H), 7.52 (s, 1H), 7.39 (d, J = 8.1 Hz, 1H), 7.33 (d, |
| | | 584.1875; found, | J = 8.1 Hz, 1H), 7.14 (d, J = 9.0 Hz, 2H), 6.90 (s, 1H), 4.84 (s, |
| | | 584.1875 | 2H), 3.99 (d, J = 2.3 Hz, 2H), 2.65 (q, J = 6.8 Hz, 1H), 2.39 (s, |
| | | | 3H), 1.20 (dd, J = 9.1, 6.9 Hz, 6H); |
| | | | ¹⁹F NMR (376 MHz, CDCl₃) δ −131.10 |
| J345 | | ESIMS m/z 582 | ¹H NMR (400 MHz, CDCl₃) δ 9.21 (s, 1H), 8.60 (s, 1H), 8.51 (t, |
| | | ([M + H]⁺) | J = 8.4 Hz, 1H), 8.15-8.03 (m, 2H), 8.00 (d, J = 8.7 Hz, 1H), |
| | | | 7.91 (dd, J = 12.0, 1.9 Hz, 1H), 7.51 (d, J = 3.0 Hz, 1H), 7.41 |
| | | | (d, J = 7.8 Hz, 1H), 7.32 (d, J = 7.9 Hz, 1H), 7.02 (s, 1H), 6.78 |
| | | | (t, J = 55.7 Hz, 1H), 4.39 (d, J = 12.6 Hz, 1H), 4.29 (d, J = 12.5 |
| | | | Hz, 1H), 3.96 (d, J = 4.1 Hz, 2H), 3.27 (s, 3H), 2.44 (s, 3H); |
| | | | ¹⁹F NMR (376 MHz, CDCl₃) δ −112.01, −131.16 |
| J346 | | HRMS-ESI (m/z) | ¹H NMR (400 MHz, CDCl₃) δ 9.21 (s, 1H), 8.60 (d, J = 2.0 Hz, |
| | | [M + H]⁺ calcd for | 1H), 8.50 (t, J = 8.3 Hz, 1H), 8.12-8.03 (m, 2H), 8.00 (dd, J = |
| | | C₂₈H₂₁F₆N₇O₃S, | 8.5, 1.5 Hz, 1H), 7.91 (dd, J = 12.0, 1.8 Hz, 1H), 7.49 (d, J = |
| | | 650.1404; found, | 3.0 Hz, 1H), 7.42 (d, J = 7.8 Hz, 1H), 7.35 (dd, J = 7.7, 1.7 Hz, |
| | | 650.1401 | 1H), 7.04 (s, 1H), 6.78 (t, J = 55.7 Hz, 1H), 4.63 (d, J = 12.4 |
| | | | Hz, 1H), 4.50 (d, J = 12.4 Hz, 1H), 3.96 (d, J = 2.7 Hz, 2H), |
| | | | 3.72 (qd, J = 8.7, 4.3 Hz, 2H), 2.43 (d, J = 20.4 Hz, 3H); |
| | | | ¹⁹F NMR (376 MHz, CDCl₃) δ −73.77, −112.02, −131.32 |
| J347 | | HRMS-ESI (m/z) | ¹H NMR (400 MHz, CDCl₃) δ 9.21 (s, 1H), 8.60 (s, 1H), 8.51 (t, |
| | | [M + H]⁺ calcd for | J = 8.3 Hz, 1H), 8.14-8.04 (m, 2H), 8.04-7.96 (m, 1H), 7.91 |
| | | C₂₉H₂₇F₃N₈O₂S, | (dd, J = 11.9, 1.9 Hz, 1H), 7.57 (d, J = 3.0 Hz, 1H), 7.33 (d, J = |
| | | 609.2003; found, | 8.8 Hz, 1H), 6.96-6.84 (m, 1H), 6.71 (d, J = 55.7 Hz, 1H), |
| | | 609.2011 | 6.36 (d, J = 2.7 Hz, 1H), 3.99 (d, J = 1.7 Hz, 2H), 2.97 (s, 6H), |
| | | | 2.59 (p, J = 6.9 Hz, 1H), 1.17 (dd, J = 9.3, 6.8 Hz, 6H); |
| | | | ¹⁹F NMR (376 MHz, CDCl₃) δ −112.01, −130.68 |
| J348 | | HRMS-ESI (m/z) | ¹H NMR (400 MHz, CDCl₃) δ 8.67 (s, 1H), 8.61 (d, J = 2.7 Hz, |
| | | [M + H]⁺ calcd for | 1H), 8.44 (t, J = 8.6 Hz, 1H), 8.18 (d, J = 8.7 Hz, 1H), 8.04 (dd, |
| | | C₂₉H₂₄F₄N₆O₂S, | J = 8.7, 2.3 Hz, 1H), 7.72-7.60 (m, 2H), 7.49 (s, 1H), 7.39 (d, |
| | | 597.169; found, | J = 8.0 Hz, 1H), 7.33 (d, J = 8.1 Hz, 1H), 6.90 (s, 1H), 6.77 (d, |
| | | 597.169 | J = 2.7 Hz, 1H), 3.99 (d, J = 2.4 Hz, 2H), 2.66 (p, J = 6.8 Hz, |
| | | | 1H), 2.39 (s, 3H), 1.20 (dd, J = 9.6, 6.9 Hz, 6H); |
| | | | ¹⁹F NMR (376 MHz, CDCl₃) δ −61.92, −130.99 |
| J349 | | HRMS-ESI (m/z) | ¹H NMR (400 MHz, CDCl₃) δ 8.67 (t, J = 1.7 Hz, 1H), 8.61 (d, J = |
| | | [M + H]⁺ calcd for | 2.7 Hz, 1H), 8.44 (t, J = 8.5 Hz, 1H), 8.18 (d, J = 8.7 Hz, 1H), |
| | | C₃₀H₂₇F₄N₇O₂S, | 8.04 (dd, J = 8.8, 2.4 Hz, 1H), 7.72-7.59 (m, 2H), 7.53 (d, J = |
| | | 626.1956; found, | 2.8 Hz, 1H), 7.33 (d, J = 8.8 Hz, 1H), 6.89 (dd, J = 8.8, 2.7 Hz, |
| | | 626.1967 | 1H), 6.77 (d, J = 2.8 Hz, 1H), 6.36 (d, J = 2.7 Hz, 1H), 3.99 (d, |
| | | | J = 1.7 Hz, 2H), 2.97 (s, 6H), 2.59 (p, J = 6.8 Hz, 1H), 1.17 (dd, |
| | | | J = 9.7, 6.8 Hz, 6H); |
| | | | ¹⁹F NMR (376 MHz, CDCl₃) δ −61.91, −130.70 |
| J350 | | ESIMS m/z 627 | ¹H NMR (400 MHz, CDCl₃) δ 8.61 (s, 1H), 8.13 (d, J = 8.2 Hz, |
| | | ([M + H]⁺) | 2H), 7.86-7.79 (m, 4H), 7.61 (dd, J = 8.7, 6.2 Hz, 2H), 7.57- |
| | | | 7.51 (m, 1H), 7.39 (t, J = 7Λ Hz, 1H), 7.28 (s, 1H), 6.94 (s, 1H), |
| | | | 4.24-4.11 (m, 1H), 4.05-3.93 (m, 2H), 3.16 (d, J = 20.0 Hz, |
| | | | 3H), 2.42 (s, 3H), 1.37 (dd, J = 11.1, 6.4 Hz, 3H); |
| | | | ¹⁹F NMR (376 MHz, CDCl₃) δ −42.66 |
| J351 | | HRMS-ESI (m/z) | ¹H NMR (400 MHz, CDCl₃) δ 9.21 (s, 1H), 8.60 (s, 1H), 8.49 (t, |
| | | [M + H]⁺ calcd for | J = 8.3 Hz, 1H), 8.16-8.04 (m, 2H), 8.00 (d, J = 8.6 Hz, 1H), |
| | | C₂₉H₂₆F₄N₈O₂S, | 7.93 (dd, J = 11.9, 1.8 Hz, 1H), 7.55 (d, J = 3.0 Hz, 1H), 7.09 |
| | | 627.1908; found, | (d, J = 14.1 Hz, 1H), 6.78 (t, J = 55.7 Hz, 1H), 6.53 (d, J = 8.3 |
| | | 627.1917 | Hz, 1H), 3.99 (d, J = 1.7 Hz, 2H), 2.87 (s, 6H), 2.58 (p, J = 7.1 |
| | | | Hz, 1H), 1.17 (t, J = 6.8 Hz, 6H) |
| J352 | | HRMS-ESI (m/z) | ¹H NMR (400 MHz, CDCl₃) δ 9.22 (s, 1H), 8.74 (dd, J = 2.2, 1.1 |
| | | [M + H]⁺ calcd for | Hz, 1H), 8.50 (t, J = 8.3 Hz, 1H), 8.19-8.07 (m, 2H), 8.00 (d, J = |
| | | C₂₉H₂₅F₅N₈O₂S, | 8.6 Hz, 1H), 7.93 (dd, J = 11.9, 1.8 Hz, 1H), 7.55 (d, J = 3.1 |
| | | 645.1814; found, | Hz, 1H), 7.09 (d, J = 14.1 Hz, 1H), 6.53 (d, J = 8.3 Hz, 1H), |
| | | 645.182 | 3.99 (d, J = 1.7 Hz, 2H), 2.87 (s, 6H), 2.58 (p, J = 7.0 Hz, 1H), |
| | | | 1.17 (t, J = 6.7 Hz, 6H); |
| | | | ¹⁹F NMR (376 MHz, CDCl₃) δ −62.08, −119.34, −130.93 |
| J353 | | HRMS-ESI (m/z) | ¹H NMR (400 MHz, CDCl₃) δ 9.21 (s, 1H), 8.60 (s, 1H), 8.46 (t, |
| | | [M + H]⁺ calcd for | J = 8.4 Hz, 1H), 8.11-8.03 (m, 2H), 8.01 (d, J = 8.8 Hz, 1H), |
| | | C₂₈H₂₁F₆N₇O₃S, | 7.91 (dd, J = 12.0, 1.8 Hz, 1H), 7.68 (d, J = 8.0 Hz, 1H), 7.44 |
| | | 650.1404; found, | (d, J = 8.0 Hz, 1H), 7.38 (d, J = 3.2 Hz, 1H), 7.02 (s, 1H), 6.78 |
| | | 650.1402 | (t, J = 55.7 Hz, 1H), 4.48 (q, J = 6.4 Hz, 1H), 4.09-3.90 (m, |
| | | | 2H), 3.43 (s, 3H), 2.47 (s, 3H); |
| | | | ¹⁹F NMR (376 MHz, CDCl₃) δ −76.42, −112.03, −131.86 |
| J354 | | HRMS-ESI (m/z) | ¹H NMR (400 MHz, CDCl₃) δ 9.21 (s, 1H), 8.60 (s, 1H), 8.48 |
| | | [M + H]⁺ calcd for | (td, J = 8.4, 5.0 Hz, 1H), 8.12-8.03 (m, 2H), 8.00 (d, J = 8.7 |
| | | C₂₉H₂₆F₃N₇O₃S, | Hz, 1H), 7.91 (dd, J = 12.0, 1.9 Hz, 1H), 7.63-7.50 (m, 2H), |
| | | 610.1843; found, | 7.46-7.33 (m, 2H), 6.92 (s, 2H), 6.71 (d, J = 55.7 Hz, 1H), |
| | | 610.185 | 4.27 (p, J = 6.7 Hz, 1H), 3.99 (d, J = 1.4 Hz, 2H), 3.47-3.15 |
| | | | (m, 3H), 2.42 (s, 3H), 1.37 (dd, J = 17.7, 6.4 Hz, 3H); |
| | | | ¹⁹F NMR (376 MHz, CDCl₃) δ −112.02, −131.65 |
| J355 | | HRMS-ESI (m/z) | ¹H NMR (400 MHz, CDCl₃) δ 9.21 (s, 1H), 8.60 (s, 1H), 8.50 (t, |
| | | [M + H]⁺ calcd for | J = 8.3 Hz, 1H), 8.12-8.04 (m, 2H), 8.04-7.97 (m, 1H), 7.92 |
| | | C₂₈H₂₄F₃N₇O₃S, | (dd, J = 11.9, 1.8 Hz, 1H), 7.54 (d, J = 3.0 Hz, 1H), 7.40 (d, J = |
| | | 596.1686; found, | 8.8 Hz, 1H), 7.08 (dd, J = 8.7, 2.7 Hz, 1H), 6.78 (t, J = 55.7 Hz, |
| | | 596.1688 | 1H), 6.62 (d, J = 2.7 Hz, 1H), 4.00 (d, J = 2.1 Hz, 2H), 3.83 (s, |
| | | | 3H), 2.63 (p, J = 6.9 Hz, 1H), 1.19 (t, J = 6.4 Hz, 6H); |
| | | | ¹⁹F NMR (376 MHz, CDCl₃) δ −112.02, −130.96 |
| J356 | | HRMS-ESI (m/z) | ¹H NMR (400 MHz, CDCl₃) δ 8.48 (s, 1H), 8.17 (d, J = 8.5 Hz, |
| | | [M + H]⁺ calcd for | 1H), 8.03 (d, J = 8.4 Hz, 1H), 7.98 (s, 1H), 7.76-7.70 (m, 2H), |
| | | C₃₁H₂₉N₇O₃S, | 7.38 (d, J = 8.1 Hz, 1H), 7.32 (d, J = 8.0 Hz, 1H), 7.16-7.11 |
| | | 580.2125; found, | (m, 2H), 7.10 (s, 1H), 6.92 (s, 1H), 4.84 (s, 2H), 3.97 (d, J = 1.0 |
| | | 580.2127 | Hz, 2H), 2.68 (p, J = 6.9 Hz, 1H), 2.39 (s, 3H), 2.25 (s, 3H), |
| | | | 1.20 (dd, J = 16.6, 6.8 Hz, 6H) |
| J357 | | HRMS-ESI (m/z) | ¹H NMR (400 MHz, CDCl₃) δ 8.48 (s, 1H), 8.18 (d, J = 8.5 Hz, |
| | | [M + H]⁺ calcd for | 1H), 8.05-8.01 (m, 1H), 7.98 (s, 1H), 7.76-7.70 (m, 2H), |
| | | C₃₂H₃₂N₈O₃S, | 7.33 (d, J = 8.8 Hz, 1H), 7.17 (s, 1H), 7.16-7.09 (m, 2H), 6.88 |
| | | 609.2391; found, | (dd, J = 8.8, 2.8 Hz, 1H), 6.38 (d, J = 2.7 Hz, 1H), 4.84 (s, 2H), |
| | | 609.24 | 3.97 (s, 2H), 2.96 (s, 6H), 2.65-2.56 (m, 1H), 2.26 (s, 3H), |
| | | | 1.18 (dd, J = 15.4, 6.9 Hz, 6H) |
| J358 | | HRMS-ESI (m/z) | ¹H NMR (400 MHz, CDCl₃) δ 8.48 (s, 1H), 8.17 (dd, J = 8.5, 3.0 |
| | | [M + H]⁺ calcd for | Hz, 1H), 8.03 (d, J = 8.5 Hz, 1H), 7.98 (s, 1H), 7.75-7.70 (m, |
| | | C₃₁H₂₉N₇O₄S, | 2H), 7.54 (dd, J = 8.0, 2.9 Hz, 1H), 7.41-7.35 (m, 1H), 7.16- |
| | | 596.2074; found, | 7.11 (m, 2H), 7.04 (d, J = 22.4 Hz, 1H), 6.94 (d, J = 2.4 Hz, |
| | | 596.2076 | 1H), 4.84 (s, 2H), 4.17 (dq, J = 18.4, 6.4 Hz, 1H), 3.98 (d, J = |
| | | | 2.4 Hz, 2H), 3.16 (d, J = 38.5 Hz, 3H), 2.42 (d, J = 2.1 Hz, 3H), |
| | | | 2.22 (d, J = 23.9 Hz, 3H), 1.38 (dd, J = 26.2, 6.4 Hz, 3H) |
| J359 | | HRMS-ESI (m/z) | ¹H NMR (400 MHz, CDCl₃) δ 8.48 (s, 1H), 8.18 (d, J = 8.5 Hz, |
| | | [M + H]⁺ calcd for | 1H), 8.03 (d, J = 8.5 Hz, 1H), 7.98 (s, 1H), 7.75-7.70 (m, 2H), |
| | | C₃₁H₂₆F₃N₇O₄S, | 7.43 (d, J = 7.8 Hz, 1H), 7.34 (d, J = 7.6 Hz, 1H), 7.16-7.11 |
| | | 650.1792; found, | (m, 2H), 7.08-7.03 (m, 2H), 4.84 (s, 2H), 4.63 (d, J = 12.4 Hz, |
| | | 650.1799 | 1H), 4.51 (d, J = 12.4 Hz, 1H), 3.94 (d, J = 2.1 Hz, 2H), 3.73 (q, |
| | | | J = 8.7 Hz, 2H), 2.45 (s, 3H), 2.26 (s, 3H); |
| | | | ¹⁹F NMR (376 MHz, CDCl₃) δ −73.75 |
| J360 | | ESIMS m/z 650 | ¹H NMR (400 MHz, CDCl₃) δ 8.48 (s, 1H), 8.19 (d, J = 8.8 Hz, |
| | | ([M + H]⁺) | 1H), 8.03 (d, J = 8.3 Hz, 1H), 7.98 (s, 1H), 7.73 (d, J = 8.4 Hz, |
| | | | 2H), 7.31-7.23 (m, 2H), 7.13 (d, J = 8.4 Hz, 2H), 7.06 (d, J = |
| | | | 8.0 Hz, 1H), 6.97 (d, J = 8.4 Hz, 1H), 4.84 (s, 2H), 4.23 (s, 2H), |
| | | | 3.96-3.88 (m, 2H), 2.66-2.47 (m, 2H), 2.38 (s, 3H), 2.27 (s, |
| | | | 3H); |
| | | | ¹⁹F NMR (376 MHz, CDCl₃) δ −64.57 |
| J361 | | ESIMS m/z 677 | ¹H NMR (500 MHz, CDCl₃) δ 8.73 (s, 1H), 8.50 (m, 1H), 8.21 |
| | | ([M + H]⁺) | (d, J = 8.6 Hz, 2H), 8.10 (d, J = 8.6 Hz, 2H), 7.99 (d, J = 8.6 |
| | | | Hz, 1H), 7.92-7.87 (m, 1H), 7.53 (dd, J = 8.1, 5.6 Hz, 1H), |
| | | | 7.46 (d, J = 9.3 Hz, 1H), 7.39 (t, J = 6.6 Hz, 1H), 6.93 (s, 1H), |
| | | | 4.15 (dd, J = 10.2, 6.4 Hz, 1H), 4.03-3.97 (m, 2H), 3.15 (d, J = |
| | | | 27.8 Hz, 3H), 2.43 (s, 3H), 1.37 (dd, J = 19.7, 6.4 Hz, 3H); |
| | | | ¹⁹F NMR (471 MHz, CDCl₃) δ −78.13, −130.82, −131.25 |
| J362 | | ESIMS m/z 673 | ¹H NMR (500 MHz, CDCl₃) δ 8.73 (d, J = 0.8 Hz, 1H), 8.20 (d, |
| | | ([M + H]⁺) | J = 8.3 Hz, 3H), 8.10 (d, J = 8.6 Hz, 2H), 8.06 (d, J = 8.6 Hz, |
| | | | 1H), 8.00 (d, J = 5.1 Hz, 1H), 7.54 (dd, J = 8.1, 4.4 Hz, 1H), |
| | | | 7.38 (t, J = 7.0 Hz, 1H), 7.06 (d, J = 25.5 Hz, 1H), 6.94 (d, J = |
| | | | 4.6 Hz, 1H), 4.22-4.14 (m, 1H), 4.00-3.96 (m, 2H), 3.17 (d, |
| | | | J = 47.8 Hz, 3H), 2.43 (d, J = 2.5 Hz, 3H), 2.23 (d, J = 29.0 Hz, |
| | | | 3H), 1.38 (dd, J = 31.8, 6.4 Hz, 3H); |
| | | | ¹⁹F NMR (471 MHz, CDCl₃) δ −78.17 |
| J363 | | ESIMS m/z 731 | ¹H NMR (500 MHz, CDCl₃) δ 8.73 (s, 1H), 8.51 (t, J = 8.4 Hz, |
| | | ([M + H]⁺) | 1H), 8.21 (d, J = 8.6 Hz, 2H), 8.10 (d, J = 8.9 Hz, 2H), 7.99 (d, |
| | | | J = 8.6 Hz, 1H), 7.90 (dd, J = 12.1, 1.9 Hz, 1H), 7.49 (s, 1H), |
| | | | 7.42 (d, J = 7.8 Hz, 1H), 7.35 (d, J = 7.9 Hz, 1H), 7.04 (s, 1H), |
| | | | 4.63 (d, J = 12.4 Hz, 1H), 4.50 (d, J = 12.4 Hz, 1H), 3.96 (d, J = |
| | | | 4.0 Hz, 2H), 3.71 (qt, J = 8.7, 4.4 Hz, 2H), 2.45 (s, 3H); |
| | | | ¹⁹F NMR (471 MHz, CDCl₃) δ −73.78, −78.14, −131.09 |
| J364 | | ESIMS m/z 690 | ¹H NMR (500 MHz, CDCl₃) δ 8.73 (s, 1H), 8.52 (t, J = 8.3 Hz, |
| | | ([M + H]⁺) | 1H), 8.21 (d, J = 8.6 Hz, 2H), 8.10 (d, J = 8.8 Hz, 2H), 7.99 (d, |
| | | | J = 8.6 Hz, 1H), 7.90 (d, J = 10.8 Hz, 1H), 7.59 (s, 1H), 7.33 (d, |
| | | | J = 8.7 Hz, 1H), 6.90 (dd, J = 8.7, 2.8 Hz, 1H), 6.36 (d, J = 2.7 |
| | | | Hz, 1H), 3.99 (d, J = 2.6 Hz, 2H), 2.97 (s, 6H), 2.58 (p, J = 6.7 |
| | | | Hz, 1H), 1.17 (dd, J = 11.3, 6.8 Hz, 6H); |
| | | | ¹⁹F NMR (471 MHz, CDCl₃) δ −78.14, −130.44 |
| J365 | | ESIMS m/z 727 | ¹H NMR (500 MHz, CDCl₃) δ 8.73 (s, 1H), 8.21 (t, J = 8.6 Hz, |
| | | ([M + H]⁺) | 3H), 8.13-8.08 (m, 2H), 8.05 (d, J = 8.5 Hz, 1H), 8.02-7.98 |
| | | | (m, 1H), 7.43 (d, J = 7.9 Hz, 1H), 7.34 (d, J = 7.9 Hz, 1H), 7.05 |
| | | | (d, J = 4.1 Hz, 2H), 4.63 (d, J = 12.4 Hz, 1H), 4.52 (d, J = 12.4 |
| | | | Hz, 1H), 3.94 (d, J = 3.1 Hz, 2H), 3.73 (q, J = 8.7 Hz, 2H), 2.45 |
| | | | (s, 3H), 2.27 (s, 3H); |
| | | | ¹⁹F NMR (471 MHz, CDCl₃) δ −73.76, −78.17 |
| J366 | | ESIMS m/z 686 | ¹H NMR (500 MHz, CDCl₃) δ 8.73 (s, 1H), 8.25-8.17 (m, 3H), |
| | | ([M + H]⁺) | 8.11 (d, J = 8.9 Hz, 2H), 8.05 (d, J = 8.6 Hz, 1H), 8.00 (s, 1H), |
| | | | 7.33 (d, J = 8.8 Hz, 1H), 7.17 (s, 1H), 6.89 (dd, J = 8.7, 2.7 Hz, |
| | | | 1H), 6.38 (d, J = 2.7 Hz, 1H), 3.97 (s, 2H), 2.97 (s, 6H), 2.65- |
| | | | 2.57 (m, 1H), 2.27 (s, 3H), 1.18 (dd, J = 19.3, 6.8 Hz, 6H); |
| | | | ¹⁹F NMR (471 MHz, CDCl₃) δ −78.17 |
| J367 | | ESIMS m/z 681 | ¹H NMR (500 MHz, CDCl₃) δ 8.61 (s, 1H), 8.13 (d, J = 8.7 Hz, |
| | | ([M + H]⁺) | 2H), 7.86-7.81 (m, 4H), 7.61 (d, J = 8.6 Hz, 2H), 7.43 (d, J = |
| | | | 7.9 Hz, 1H), 7.35 (d, J = 7.8 Hz, 1H), 7.29 (s, 1H), 7.05 (s, 1H), |
| | | | 4.62 (d, J = 12.3 Hz, 1H), 4.50 (d, J = 12.4 Hz, 1H), 3.95 (d, J = |
| | | | 3.4 Hz, 2H), 3.73 (dd, J = 8.8, 2.5 Hz, 1H), 3.70-3.62 (m, 1H), |
| | | | 2.44 (s, 3H); |
| | | | ¹⁹F NMR (471 MHz, CDCl₃) δ −42.66, −73.77 |
| J368 | | ESIMS m/z 640 | ¹H NMR (500 MHz, CDCl₃) δ 8.61 (s, 1H), 8.13 (d, J = 8.6 Hz, |
| | | ([M + H]⁺) | 2H), 7.86-7.79 (m, 4H), 7.62 (d, J = 8.6 Hz, 2H), 7.43 (s, 1H), |
| | | | 7.34 (d, J = 8.9 Hz, 1H), 6.90 (dd, J = 8.8, 2.7 Hz, 1H), 6.37 (d, |
| | | | J = 2.7 Hz, 1H), 3.98 (d, J = 3.7 Hz, 2H), 2.97 (s, 6H), 2.60 (p, |
| | | | J = 6.8 Hz, 1H), 1.17 (t, J = 7.0 Hz, 6H); |
| | | | ¹⁹F NMR (471 MHz, CDCl₃) δ −42.65 |
| J369 | | ESIMS m/z 731 | ¹H NMR (500 MHz, CDCl₃) δ 8.73 (s, 1H), 8.53 (t, J = 8.4 Hz, |
| | | ([M + H]⁺) | 1H), 8.21 (d, J = 8.5 Hz, 2H), 8.10 (d, J = 8.6 Hz, 2H), 7.99 (d, |
| | | | J = 8.8 Hz, 1H), 7.90 (d, J = 11.8 Hz, 1H), 7.52 (s, 1H), 7.29 (d, |
| | | | J = 9.0 Hz, 1H), 7.03 (s, 1H), 6.97 (d, J = 8.5 Hz, 1H), 4.22 (q, |
| | | | J = 5.9 Hz, 2H), 3.94 (d, J = 2.4 Hz, 2H), 2.59-2.48 (m, 2H), |
| | | | 2.39 (s, 3H); |
| | | | ¹⁹F NMR (471 MHz, CDCl₃) δ −64.60, −78.14, −131.20 |
| J370 | | ESIMS m/z 690 | ¹H NMR (500 MHz, CDCl₃) δ 8.73 (s, 1H), 8.52 (t, J = 8.4 Hz, |
| | | ([M + H]⁺) | 1H), 8.21 (d, J = 8.4 Hz, 2H), 8.10 (d, J = 8.5 Hz, 2H), 7.99 (d, |
| | | | J = 8.5 Hz, 1H), 7.90 (d, J = 11.7 Hz, 1H), 7.59 (s, 1H), 7.24 (s, |
| | | | 1H), 6.86-6.82 (m, 1H), 6.40 (s, 1H), 3.98 (d, J = 2.7 Hz, 2H), |
| | | | 2.97 (s, 6H), 2.31 (t, J = 8.1 Hz, 2H), 1.58-1.53 (m, 2H), 0.91 |
| | | | (t, J = 7.1 Hz, 3H); |
| | | | ¹⁹F NMR (471 MHz, CDCl₃) δ −78.14, −130.50 |
| J371 | | ESIMS m/z 708 | ¹H NMR (500 MHz, CDCl₃) δ 8.74 (s, 1H), 8.51 (t, J = 8.2 Hz, |
| | | ([M + H]⁺) | 1H), 8.21 (d, J = 8.4 Hz, 2H), 8.10 (d, J = 8.6 Hz, 2H), 7.99 (d, |
| | | | J = 8.5 Hz, 1H), 7.91 (d, J = 12.1 Hz, 1H), 7.56 (s, 1H), 7.09 (d, |
| | | | J = 14.0 Hz, 1H), 6.53 (d, J = 8.2 Hz, 1H), 3.99 (s, 2H), 2.88 (s, |
| | | | 6H), 2.63-2.55 (m, 1H), 1.17 (t, J = 7.4 Hz, 6H); |
| | | | ¹⁹F NMR (471 MHz, CDCl₃) δ −78.13, −119.33, −130.76 |
| J372 | | ESIMS m/z 742 | ¹H NMR (500 MHz, CDCl₃) δ 8.74 (s, 1H), 8.50 (t, J = 8.3 Hz, |
| | | ([M + H]⁺) | 1H), 8.21 (d, J = 8.6 Hz, 2H), 8.10 (d, J = 8.5 Hz, 2H), 8.00 (d, |
| | | | J = 8.7 Hz, 1H), 7.92 (d, J = 11.8 Hz, 1H), 7.83 (dd, J = 8.8, 2.0 |
| | | | Hz, 1H), 7.57 (d, J = 2.1 Hz, 1H), 7.48 (d, J = 9.1 Hz, 1H), 7.15 |
| | | | (d, J = 8.7 Hz, 1H), 4.43-4.26 (m, 2H), 3.96 (s, 2H), 2.67- |
| | | | 2.55 (m, 2H); |
| | | | ¹⁹F NMR (471 MHz, CDCl₃) δ −64.55, −78.13, −131.19 |
| J373 | | ESIMS m/z 727 | ¹H NMR (500 MHz, CDCl₃) δ 8.73 (s, 1H), 8.22 (dd, J = 17.4, |
| | | ([M + H]⁺) | 8.6 Hz, 3H), 8.11 (d, J = 8.9 Hz, 2H), 8.05 (d, J = 8.6 Hz, 1H), |
| | | | 8.00 (s, 1H), 7.28 (d, J = 7.3 Hz, 1H), 7.10 (s, 1H), 7.05 (s, 1H), |
| | | | 6.97 (d, J = 8.5 Hz, 1H), 4.23 (t, J = 6.3 Hz, 2H), 3.92 (d, J = |
| | | | 2.6 Hz, 2H), 2.55 (dt, J = 11.4, 6.1 Hz, 2H), 2.38 (s, 3H), 2.29 |
| | | | (s, 3H); |
| | | | ¹⁹F NMR (471 MHz, CDCl₃) δ −64.57, −78.17 |
| J374 | | ESIMS m/z 686 | ¹H NMR (500 MHz, CDCl₃) δ 8.73 (s, 1H), 8.22 (dd, J = 14.6, |
| | | ([M + H]⁺) | 8.5 Hz, 3H), 8.11 (d, J = 8.6 Hz, 2H), 8.05 (d, J = 8.7 Hz, 1H), |
| | | | 8.00 (s, 1H), 7.24 (s, 1H), 7.17 (s, 1H), 6.86-6.81 (m, 1H), |
| | | | 6.42 (d, J = 2.6 Hz, 1H), 3.97 (d, J = 2.1 Hz, 2H), 2.97 (s, 6H), |
| | | | 2.33 (q, J = 7.7 Hz, 2H), 2.28 (s, 3H), 1.60-1.53 (m, 2H), 0.92 |
| | | | (t, J = 7.3 Hz, 3H); |
| | | | ¹⁹F NMR (471 MHz, CDCl₃) δ −78.17 |
| J375 | | ESIMS m/z 704 | ¹H NMR (500 MHz, CDCl₃) δ 8.73 (s, 1H), 8.25-8.17 (m, 3H), |
| | | ([M + H]⁺) | 8.08 (dd, J = 25.0, 8.5 Hz, 3H), 8.01 (s, 1H), 7.13 (s, 1H), 7.08 |
| | | | (d, J = 14.1 Hz, 1H), 6.55 (d, J = 8.3 Hz, 1H), 3.98 (s, 2H), 2.87 |
| | | | (s, 6H), 2.63-2.57 (m, 1H), 2.28 (s, 3H), 1.18 (dd, J = 13.5, |
| | | | 6.8 Hz, 6H); |
| | | | ¹⁹F NMR (471 MHz, CDCl₃) δ −78.17, −119.51 |
| J376 | | ESIMS m/z 738 | ¹H NMR (400 MHz, CDCl₃) δ 8.74 (s, 1H), 8.31-8.17 (m, 3H), |
| | | ([M + H]⁺) | 8.11 (d, J = 8.8 Hz, 2H), 8.06 (d, J = 8.5 Hz, 1H), 8.01 (s, 1H), |
| | | | 7.82 (dd, J = 8.7, 2.1 Hz, 1H), 7.59 (d, J = 2.1 Hz, 1H), 7.14 (d, |
| | | | J = 8.7 Hz, 1H), 7.07 (s, 1H), 4.33 (q, J = 6.1 Hz, 2H), 3.94 (s, |
| | | | 2H), 2.62 (dt, J = 10.5, 5.8 Hz, 2H), 2.30 (s, 3H); |
| | | | ¹⁹F NMR (376 MHz, CDCl₃) δ −64.53, −78.17 |
| J377 | | HRMS-ESI (m/z) | ¹H NMR (400 MHz, CDCl₃) δ 9.22 (s, 1H), 8.74 (d, J = 1.7 Hz, |
| | | [M + H]⁺ calcd for | 1H), 8.46 (t, J = 8.3 Hz, 1H), 8.19-8.06 (m, 2H), 8.06-7.96 |
| | | C₂₈H₂₀F₇N₇O₃S, | (m, 1H), 7.91 (dd, J = 11.9, 1.9 Hz, 1H), 7.68 (d, J = 8.0 Hz, |
| | | 668.1309; found, | 1H), 7.41 (dd, J = 23.1, 5.7 Hz, 2H), 7.02 (d, J = 1.6 Hz, 1H), |
| | | 668.1317 | 4.48 (q, J = 6.3 Hz, 1H), 4.08-3.91 (m, 2H), 3.43 (s, 3H), 2.47 |
| | | | (s, 3H); |
| | | | ¹⁹F NMR (376 MHz, CDCl₃) δ −62.09, −76.41, −131.81 |
| J378 | | HRMS-ESI (m/z) | ¹H NMR (400 MHz, CDCl₃) δ 9.22 (s, 1H), 8.78-8.67 (m, 1H), |
| | | [M + H]⁺ calcd for | 8.49 (td, J = 8.4, 4.9 Hz, 1H), 8.18-8.05 (m, 2H), 8.00 (d, J = |
| | | C₂₉H₂₅F₄N₇O₃S, | 8.6 Hz, 1H), 7.91 (dd, J = 12.0, 1.8 Hz, 1H), 7.61-7.48 (m, |
| | | 628.1748; found, | 1H), 7.47-7.33 (m, 2H), 6.92 (d, J = 1.9 Hz, 1H), 4.26 (dq, J = |
| | | 628.1753 | 13.5, 6.5 Hz, 1H), 4.00 (d, J = 1.4 Hz, 2H), 3.45-3.21 (m, 2H), |
| | | | 2.42 (s, 3H), 1.37 (dd, J = 17.6, 6.5 Hz, 3H), 1.14 (td, J = 6.9, |
| | | | 4.6 Hz, 3H); |
| | | | ¹⁹F NMR (376 MHz, CDCl₃) δ −62.08, −131.59 |
| J379 | | ESIMS m/z 674 | ¹H NMR (500 MHz, CDCl₃) δ 8.68 (s, 1H), 8.51 (t, J = 7.9 Hz, |
| | | ([M + H]⁺) | 1H), 8.04 (d, J = 8.5 Hz, 2H), 7.97 (d, J = 8.1 Hz, 3H), 7.90 (d, |
| | | | J = 12.0 Hz, 1H), 7.59 (s, 1H), 7.33 (d, J = 8.9 Hz, 1H), 6.90 (d, |
| | | | J = 8.8 Hz, 1H), 6.36 (s, 1H), 3.99 (s, 2H), 2.97 (s, 6H), 2.62- |
| | | | 2.55 (m, 1H), 1.17 (dd, J = 11.6, 6.9 Hz, 6H); |
| | | | ¹⁹F NMR (471 MHz, CDCl₃) δ −74.21, −130.57 |
| J380 | | ESIMS m/z 715 | 1H NMR (500 MHz, CDCl₃) δ 8.68 (s, 1H), 8.50 (t, J = 8.2 Hz, |
| | | ([M + H]⁺) | 1H), 8.04 (d, J = 8.4 Hz, 2H), 8.01-7.93 (m, 3H), 7.90 (d, J = |
| | | | 12.1 Hz, 1H), 7.49 (s, 1H), 7.38 (s, 2H), 7.04 (s, 1H), 4.63 (d, J = |
| | | | 12.2 Hz, 1H), 4.50 (d, J = 12.4 Hz, 1H), 3.96 (d, J = 4.0 Hz, |
| | | | 2H), 3.74-3.67 (m, 2H), 2.45 (s, 3H); |
| | | | ¹⁹F NMR (471 MHz, CDCl₃) δ −73.78, −74.21, −131.21 |
| J381 | | HRMS-ESI (m/z) | ¹H NMR (400 MHz, CDCl₃) δ 8.85 (d, J = 2.8 Hz, 1H), 8.45 (dd, |
| | | [M + H]⁺ calcd for | J = 11.3, 8.1 Hz, 2H), 7.94 (d, J = 9.2 Hz, 1H), 7.66 (d, J = 8.3 |
| | | C₂₈H₂₃F₄N₇O₃S, | Hz, 2H), 7.62-7.46 (m, 1H), 7.46-7.33 (m, 2H), 6.93 (s, 1H), |
| | | 614.1592; found, | 6.86 (dd, J = 2.9, 1.1 Hz, 1H), 4.25-4.09 (m, 1H), 4.00 (dd, J = |
| | | 614.1596 | 3.0, 1.8 Hz, 2H), 3.16 (d, J = 22.8 Hz, 3H), 2.43 (s, 3H), 1.37 |
| | | | (dd, J = 16.4, 6.4 Hz, 3H) |
| J382 | | ESIMS m/z 715 | ¹H NMR (500 MHz, CDCl₃) δ 8.68 (s, 1H), 8.51 (t, J = 8.3 Hz, |
| | | ([M + H]⁺) | 1H), 8.04 (d, J = 8.4 Hz, 2H), 7.97 (d, J = 8.1 Hz, 3H), 7.90 (d, |
| | | | J = 11.7 Hz, 1H), 7.52 (s, 1H), 7.38 (s, 1H), 7.03 (s, 1H), 6.97 |
| | | | (d, J = 8.5 Hz, 1H), 4.25-4.18 (m, 2H), 3.94 (s, 2H), 2.54 (s, |
| | | | 2H), 2.38 (s, 3H); |
| | | | ¹⁹F NMR (471 MHz, CDCl₃) δ −64.60, −74.21 , −131.32 |
| J383 | | ESIMS m/z 674 | ¹H NMR (500 MHz, CDCl₃) δ 8.68 (s, 1H), 8.51 (d, J = 7.9 Hz, |
| | | ([M + H]⁺) | 1H), 8.04 (d, J = 8.1 Hz, 2H), 7.97 (d, J = 8.0 Hz, 2H), 7.90 (d, |
| | | | J = 12.1 Hz, 1H), 7.86 (s, 1H), 7.59 (s, 1H), 7.38 (s, 1H), 6.83 |
| | | | (d, J = 8.8 Hz, 1H), 6.40 (s, 1H), 3.98 (s, 2H), 2.97 (s, 6H), 2.31 |
| | | | (t, J = 8.0 Hz, 2H), 1.60-1.50 (m, 2H), 0.91 (t, J = 7.3 Hz, 3H); |
| | | | ¹⁹F NMR (471 MHz, CDCl₃) δ −74.21, −130.63 |
| J384 | | ESIMS m/z 692 | ¹H NMR (500 MHz, CDCl₃) δ 8.68 (s, 1H), 8.50 (t, J = 8.1 Hz, |
| | | ([M + H]⁺) | 1H), 8.04 (d, J = 8.3 Hz, 2H), 8.02-7.96 (m, 2H), 7.91 (d, J = |
| | | | 11.7 Hz, 1H), 7.56 (s, 1H), 7.38 (s, 1H), 7.08 (d, J = 14.3 Hz, |
| | | | 1H), 6.53 (d, J = 8.2 Hz, 1H), 3.99 (d, J = 2.4 Hz, 2H), 2.87 (s, |
| | | | 6H), 2.58 (s, 1H), 1.17 (t, J = 7.5 Hz, 6H); |
| | | | ¹⁹F NMR (471 MHz, CDCl₃) δ −74.20, −119.35, −130.88 |
| J385 | | ESIMS m/z 726 | ¹H NMR (500 MHz, CDCl₃) δ 8.68 (s, 1H), 8.49 (t, J = 8.3 Hz, |
| | | ([M + H]⁺) | 1H), 8.04 (d, J = 8.3 Hz, 2H), 8.03-7.94 (m, 3H), 7.92 (d, J = |
| | | | 12.1 Hz, 1H), 7.82 (d, J = 8.8 Hz, 1H), 7.57 (d, J = 2.2 Hz, 1H), |
| | | | 7.48 (s, 1H), 7.14 (d, J = 8.8 Hz, 1H), 4.31 (dd, J = 13.7, 6.5 |
| | | | Hz, 2H), 3.96 (s, 2H), 2.68-2.55 (m, 2H); |
| | | | ¹⁹F NMR (471 MHz, CDCl₃) δ −64.56, −74.20, −131.31 |
| J386 | | ESIMS m/z 715 | ¹H NMR (500 MHz, CDCl₃) δ 8.68 (s, 1H), 8.46 (t, J = 8.1 Hz, |
| | | ([M + H]⁺) | 1H), 8.04 (d, J = 8.6 Hz, 2H), 8.02-7.95 (m, 2H), 7.90 (d, J = |
| | | | 11.8 Hz, 1H), 7.68 (d, J = 8.1 Hz, 1H), 7.44 (d, J = 8.1 Hz, 1H), |
| | | | 7.38 (s, 2H), 7.02 (s, 1H), 4.47 (d, J = 6.5 Hz, 1H), 4.07-3.92 |
| | | | (m, 2H), 3.43 (s, 3H), 2.47 (s, 3H); |
| | | | ¹⁹F NMR (471 MHz, CDCl₃) δ −74.20, −76.39, −131.75 |
| J387 | | ESIMS m/z 681 | ¹H NMR (500 MHz, CDCl₃) δ 8.61 (s, 1H), 8.13 (d, J = 8.4 Hz, |
| | | ([M + H]⁺) | 2H), 7.83 (t, J = 7.5 Hz, 4H), 7.62 (d, J = 8.3 Hz, 2H), 7.30 (d, J = |
| | | | 11.9 Hz, 2H), 7.04 (s, 1H), 6.97 (d, J = 8.5 Hz, 1H), 4.28- |
| | | | 4.18 (m, 2H), 3.93 (s, 2H), 2.61-2.48 (m, 2H), 2.38 (s, 3H); |
| | | | ¹⁹F NMR (471 MHz, CDCl₃) δ −42.67, −64.59 |
| J388 | | ESIMS m/z 640 | ¹H NMR (500 MHz, CDCl₃) δ 8.61 (s, 1H), 8.13 (d, J = 8.3 Hz, |
| | | ([M + H]⁺) | 2H), 7.83 (q, J = 8.6 Hz, 5H), 7.63 (d, J = 8.3 Hz, 2H), 7.43 (s, |
| | | | 1H), 6.84 (s, 1H), 6.42 (s, 1H), 3.97 (d, J = 3.6 Hz, 2H), 2.97 (s, |
| | | | 6H), 2.32 (t, J = 7.9 Hz, 2H), 1.60-1.55 (m, 2H), 0.91 (t, J = |
| | | | 7.3 Hz, 3H); |
| | | | ¹⁹F NMR (471 MHz, CDCl₃) δ −42.67 |
| J389 | | ESIMS m/z 658 | ¹H NMR (500 MHz, CDCl₃) δ 8.61 (s, 1H), 8.14 (d, J = 8.3 Hz, |
| | | ([M + H]⁺) | 2H), 7.84 (t, J = 7.4 Hz, 4H), 7.64 (d, J = 8.3 Hz, 2H), 7.37 (s, |
| | | | 1H), 7.08 (d, J = 14.1 Hz, 1H), 6.54 (d, J = 8.4 Hz, 1H), 3.98 (s, |
| | | | 2H), 2.87 (s, 6H), 2.60 (s, 1H), 1.17 (t, J = 5.9 Hz, 6H); |
| | | | ¹⁹F NMR (471 MHz, CDCl₃) δ −42.66, −119.30 |
| J390 | | HRMS-ESI (m/z) | ¹H NMR (400 MHz, CDCl₃) δ 8.85 (d, J = 2.8 Hz, 1H), 8.55- |
| | | [M + H]⁺ calcd for | 8.37 (m, 2H), 7.94 (d, J = 9.1 Hz, 1H), 7.74-7.59 (m, 2H), |
| | | C₂₈H₂₀F₇N₇O₃S, | 7.52-7.39 (m, 2H), 7.39-7.30 (m, 1H), 7.09-6.99 (m, 1H), |
| | | 668.1309; found, | 6.86 (d, J = 2.8 Hz, 1H), 4.63 (d, J = 12.4 Hz, 1H), 4.50 (d, J = |
| | | 668.131 | 12.4 Hz, 1H), 3.96 (d, J = 2.6 Hz, 2H), 3.72 (qd, J = 8.7, 3.6 |
| | | | Hz, 2H), 2.45 (s, 3H); |
| | | | ¹⁹F NMR (376 MHz, CDCl₃) δ −66.52, −73.78, −131.10 |
| J391 | | HRMS-ESI (m/z) | ¹H NMR (400 MHz, CDCl₃) δ 8.85 (d, J = 2.8 Hz, 1H), 8.46 (dd, |
| | | [M + H]⁺ calcd for | J = 15.9, 8.6 Hz, 2H), 7.94 (d, J = 9.2 Hz, 1H), 7.72-7.62 (m, |
| | | C₂₉H₂₆F₄N₈O₂S, | 2H), 7.55 (d, J = 2.9 Hz, 1H), 7.34 (d, J = 8.8 Hz, 1H), 6.94- |
| | | 627.1908; found, | 6.80 (m, 2H), 6.36 (d, J = 2.7 Hz, 1H), 3.99 (d, J = 1.7 Hz, 2H), |
| | | 627.1917 | 2.97 (s, 6H), 2.59 (p, J = 6.8 Hz, 1H), 1.17 (dd, J = 9.4, 6.8 Hz, |
| | | | 6H); |
| | | | ¹⁹F NMR (376 MHz, CDCl₃) δ −66.52, −130.45 |
| J392 | | ESIMS m/z 692 | ¹H NMR (500 MHz, CDCl₃) δ 8.61 (s, 1H), 8.15 (d, J = 8.4 Hz, |
| | | ([M + H]⁺) | 2H), 7.83 (m, 6H), 7.63 (d, J = 8.4 Hz, 2H), 7.58 (s, 1H), 7.13 |
| | | | (d, J = 8.7 Hz, 1H), 4.32 (s, 2H), 3.94 (s, 2H), 2.61 (s, 2H); |
| | | | ¹⁹F NMR (471 MHz, CDCl₃) δ −42.66, −64.56 |
| J393 | | ESIMS m/z 629 | ¹H NMR (400 MHz, CDCl₃) δ 9.46 (s, 1H), 8.53 (t, J = 8.3 Hz, |
| | | ([M + H]⁺) | 1H), 8.37 (d, J = 9.1 Hz, 1H), 8.03 (dd, J = 18.7, 8.7 Hz, 2H), |
| | | | 7.91 (dd, J = 11.8, 1.8 Hz, 1H), 7.59 (d, J = 3.0 Hz, 1H), 7.34 |
| | | | (d, J = 8.8 Hz, 1H), 6.90 (dd, J = 8.8, 2.7 Hz, 1H), 6.36 (d, J = |
| | | | 2.7 Hz, 1H), 4.00 (d, J = 1.7 Hz, 2H), 2.97 (s, 6H), 2.58 (p, J = |
| | | | 6.9 Hz, 1H), 1.17 (dd, J = 9.1, 6.9 Hz, 6H); |
| | | | ¹⁹F NMR (376 MHz, CDCl₃) δ −66.55, −130.37 |
| J394 | | HRMS-ESI (m/z) | ¹H NMR (400 MHz, CDCl₃) δ 9.46 (s, 1H), 8.52 (t, J = 8.3 Hz, |
| | | [M + H]⁺ calcd for | 1H), 8.37 (d, J = 9.1 Hz, 1H), 8.03 (dd, J = 18.2, 8.9 Hz, 2H), |
| | | C₂₇H₁₉F₇N₈O₃S, | 7.92 (dd, J = 11.9, 1.9 Hz, 1H), 7.50 (d, J = 3.1 Hz, 1H), 7.42 |
| | | 669.1262; found, | (d, J = 7.8 Hz, 1H), 7.38-7.31 (m, 1H), 7.05 (d, J = 1.7 Hz, |
| | | 669.1271 | 1H), 4.63 (d, J = 12.5 Hz, 1H), 4.50 (d, J = 12.4 Hz, 1H), 3.97 |
| | | | (d, J = 2.7 Hz, 2H), 3.72 (qd, J = 8.7, 3.6 Hz, 2H), 2.45 (s, 3H); |
| | | | ¹⁹F NMR (376 MHz, CDCl₃) δ −66.55, −73.78, −131.02 |
| J395 | | HRMS-ESI (m/z) | ¹H NMR (400 MHz, CDCl₃) δ 11.44 (s, 1H), 8.85 (d, J = 8.9 Hz, |
| | | [M + H]⁺ calcd for | 1H), 8.68 (d, J = 2.0 Hz, 1H), 8.57 (s, 1H), 8.34 (d, J = 9.1 Hz, |
| | | C₃₁H₂₇F₃N₆O₄S, | 1H), 7.81 (d, J = 9.0 Hz, 2H), 7.40 (dd, J = 8.2, 5.2 Hz, 3H), |
| | | 637.1839; found, | 7.30 (d, J = 8.2 Hz, 1H), 6.89 (s, 1H), 3.99 (s, 2H), 2.71 (s, 3H), |
| | | 637.1834 | 2.69 (d, J = 7.1 Hz, 1H), 2.38 (s, 3H), 1.22 (dd, J = 38.4, 6.9 |
| | | | Hz, 6H); |
| | | | ¹⁹F NMR (376 MHz, CDCl₃) δ −58.02 |
| J396 | | HRMS-ESI (m/z) | ¹H NMR (400 MHz, CDCl₃) δ 11.43 (s, 1H), 8.85 (d, J = 9.1 Hz, |
| | | [M + H]⁺ calcd for | 1H), 8.67 (d, J = 2.0 Hz, 1H), 8.57 (s, 1H), 8.34 (d, J = 9.0 Hz, |
| | | C₃₂H₃₀F₃N₇O₄S, | 1H), 7.84-7.77 (m, 2H), 7.40 (d, J = 8.5 Hz, 2H), 7.33 (d, J = |
| | | 666.2105; found, | 8.8 Hz, 1H), 6.87 (dd, J = 8.8, 2.7 Hz, 1H), 6.35 (d, J = 2.7 Hz, |
| | | 666.2107 | 1H), 3.99 (s, 2H), 2.95 (s, 6H), 2.71 (s, 3H), 2.60 (dt, J = 13.5, |
| | | | 6.6 Hz, 1H), 1.20 (dd, J = 38.9, 7.0 Hz, 6H); |
| | | | ¹⁹F NMR (376 MHz, CDCl₃) δ −58.01 |
| J397 | | HRMS-ESI (m/z) | ¹H NMR (400 MHz, CDCl₃) δ 11.45 (d, J = 16.6 Hz, 1H), 8.85 |
| | | [M + H]⁺ calcd for | (d, J = 8.9 Hz, 1H), 8.67 (dd, J = 5.1, 2.0 Hz, 1H), 8.57 (d, J = |
| | | C₃₁H₂₇F₃N₆O₅S, | 0.7 Hz, 1H), 8.34 (d, J = 8.9 Hz, 1H), 7.83-7.77 (m, 2H), 7.55 |
| | | 653.1788; found, | (dd, J = 8.0, 6.1 Hz, 1H), 7.44-7.35 (m, 3H), 6.96-6.88 (m, |
| | | 653.1786 | 1H), 4.18 (dq, J = 26.3, 6.4 Hz, 1H), 3.99 (d, J = 2.8 Hz, 2H), |
| | | | 3.17 (d, J = 55.0 Hz, 3H), 2.70 (d, J = 10.6 Hz, 3H), 2.41 (d, J = |
| | | | 1.9 Hz, 3H), 1.40 (dd, J = 50.5, 6.4 Hz, 3H); |
| | | | ¹⁹F NMR (376 MHz, CDCl₃) δ −58.01 |
| J398 | | HRMS-ESI (m/z) | ¹H NMR (400 MHz, CDCl₃) δ 11.44 (s, 1H), 8.84 (d, J = 8.7 Hz, |
| | | [M + H]⁺ calcd for | 1H), 8.68 (d, J = 2.0 Hz, 1H), 8.57 (s, 1H), 8.35 (d, J = 8.9 Hz, |
| | | C₃₁H₂₄F₆N₆O₅S, | 1H), 7.84-7.77 (m, 2H), 7.41 (t, J = 7.9 Hz, 3H), 7.33 (d, J = |
| | | 707.1506; found, | 7.8 Hz, 1H), 7.04 (s, 1H), 4.72-4.47 (m, 2H), 3.96 (d, J = 1.3 |
| | | 707.1508 | Hz, 2H), 3.79-3.62 (m, 2H), 2.71 (s, 3H), 2.44 (s, 3H); |
| | | | ¹⁹F NMR (376 MHz, CDCl₃) δ −58.02, −73.79 |
| J399 | | HRMS-ESI (m/z) | ¹H NMR (400 MHz, CDCl₃) δ 11.42 (s, 1H), 8.85 (d, J = 8.9 Hz, |
| | | [M + H]⁺ calcd for | 1H), 8.68 (d, J = 2.0 Hz, 1H), 8.57 (s, 1H), 8.34 (d, J = 8.9 Hz, |
| | | C₃₁H₂₄F₆N₆O₅S, | 1H), 7.84-7.76 (m, 2H), 7.42-7.38 (m, 2H), 7.28 (d, J = 1.5 |
| | | 707.1506; found, | Hz, 1H), 7.04 (d, J = 2.2 Hz, 1H), 6.98 (d, J = 8.5 Hz, 1H), 4.25 |
| | | 707.1506 | (t, J = 6.4 Hz, 2H), 3.94 (d, J = 0.9 Hz, 2H), 2.72 (s, 3H), 2.53 |
| | | | (qt, J = 10.5, 6.4 Hz, 2H), 2.38 (s, 3H); |
| | | | ¹⁹F NMR (376 MHz, CDCl₃) δ −58.02, −64.62 |
| J400 | | ESIMS m/z 650 | ¹H NMR (400 MHz, CDCl₃) δ 9.22 (s, 1H), 8.74 (d, J = 1.9 Hz, |
| | | ([M + H]⁺) | 1H), 8.50 (t, J = 8.3 Hz, 1H), 8.20-8.07 (m, 2H), 8.07-7.84 |
| | | | (m, 2H), 7.50 (d, J = 3.1 Hz, 1H), 7.47-7.29 (m, 2H), 7.04 (s, |
| | | | 1H), 5.80 (tt, J = 55.4, 4.1 Hz, 1H), 4.67-4.37 (m, 2H), 4.06- |
| | | | 3.87 (m, 2H), 3.57 (td, J = 14.0, 4.0 Hz, 2H), 2.45 (s, 3H); |
| | | | ¹⁹F NMR (376 MHz, CDCl₃) δ −62.08, −124.97, −131.20 |
| J401 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 8.02-7.93 (m, 1H), 7.88 (dd, J = 12.0, 1.9 Hz, 1H), 7.84- |
| | | C₂₉H₂₂F₆N₆O₄S, | 7.74 (m, 2H), 7.48 (d, J = 3.0 Hz, 1H), 7.44-7.31 (m, 4H), |
| | | 665.14; found, | 5.80 (tt, J = 55.4, 4.1 Hz, 1H), 4.64-4.34 (m, 2H), 4.04-3.87 |
| | | 665.1402 | (m, 2H), 3.57 (td, J = 13.9, 4.0 Hz, 2H), 2.45 (s, 3H); |
| | | | ¹⁹F NMR (376 MHz, CDCl₃) δ −58.02, −124.92, −131.30 |
| J402 | | ESIMS m/z 738 | ¹H NMR (400 MHz, CDCl₃) δ 8.76 (s, 1H), 8.66 (d, J = 8.8 Hz, |
| | | ([M + H]⁺) | 1H), 8.39 (d, J = 9.4 Hz, 2H), 8.22 (d, J = 8.6 Hz, 2H), 8.15- |
| | | | 8.08 (m, 2H), 7.74 (s, 1H), 7.30 (s, 1H), 7.01 (s, 1H), 6.97 (d, J = |
| | | | 8.5 Hz, 1H), 4.29-4.18 (m, 2H), 3.95 (d, J = 1.1 Hz, 2H), |
| | | | 2.55 (dt, J = 10.9, 5.8 Hz, 2H), 2.38 (s, 3H); |
| | | | ¹⁹F NMR (376 MHz, CDCl₃) δ −64.52, −78.10 |
| J403 | | ESIMS m/z 665 | ¹H NMR (400 MHz, CDCl₃) δ 8.51 (s, 1H), 8.22 (d, J = 8.8 Hz, |
| | | ([M + H]⁺) | 2H), 7.66 (s, 4H), 7.43 (d, J = 7.8 Hz, 1H), 7.35 (d, J = 7.9 Hz, |
| | | | 1H), 7.30 (d, J = 8.4 Hz, 3H), 7.05 (s, 1H), 4.62 (d, J = 12.3 Hz, |
| | | | 1H), 4.50 (d, J = 12.2 Hz, 1H), 3.96 (d, J = 2.1 Hz, 2H), 3.82- |
| | | | 3.65 (m, 2H), 2.45 (s, 3H); |
| | | | ¹⁹F NMR (376 MHz, CDCl₃) δ −57.70, −73.76 |
| J404 | | ESIMS m/z 665 | ¹H NMR (400 MHz, CDCl₃) δ 8.51 (s, 1H), 8.22 (d, J = 8.9 Hz, |
| | | ([M + H]⁺) | 2H), 7.66 (s, 4H), 7.34-7.27 (m, 4H), 7.04 (s, 1H), 6.97 (d, J = |
| | | | 8.5 Hz, 1H), 4.22 (td, J = 6.3, 2.8 Hz, 2H), 3.94 (d, J = 1.7 Hz, |
| | | | 2H), 2.55 (dt, J = 11.0, 6.1 Hz, 2H), 2.38 (s, 3H); |
| | | | ¹⁹F NMR (376 MHz, CDCl₃) δ −57.70, −64.57 |
| J405 | | ESIMS m/z 665 | ¹H NMR (400 MHz, CDCl₃) δ 8.51 (s, 1H), 8.21 (d, J = 8.8 Hz, |
| | | ([M + H]⁺) | 2H), 7.65 (m, 5H), 7.43 (d, J = 8.1 Hz, 1H), 7.31 (d, J = 8.3 Hz, |
| | | | 2H), 7.21 (s, 1H), 7.02 (s, 1H), 4.45 (t, J = 6.3 Hz, 1H), 4.07- |
| | | | 3.91 (m, 2H), 3.44 (s, 3H), 2.46 (s, 3H); |
| | | | ¹⁹F NMR (376 MHz, CDCl₃) δ −57.70, −76.14 |
| J406 | | ESIMS m/z 676 | ¹H NMR (400 MHz, CDCl₃) δ 8.52 (s, 1H), 8.22 (d, J = 8.8 Hz, |
| | | ([M + H]⁺) | 2H), 7.81 (dd, J = 8.6, 2.1 Hz, 1H), 7.68 (s, 4H), 7.58 (d, J = |
| | | | 2.1 Hz, 1H), 7.33-7.27 (m, 3H), 7.14 (d, J = 8.7 Hz, 1H), 4.32 |
| | | | (dt, J = 6.2, 3.6 Hz, 2H), 3.95 (s, 2H), 2.62 (dq, J = 11.0, 5.8, |
| | | | 4.9 Hz, 2H); |
| | | | ¹⁹F NMR (376 MHz, CDCl₃) δ −57.70, −64.53 |
| J407 | | ESIMS m/z 606 | ¹H NMR (400 MHz, CDCl₃) δ 8.52 (s, 1H), 8.22 (d, J = 8.8 Hz, |
| | | ([M + H]⁺) | 2H), 7.79 (dd, J = 8.2, 1.7 Hz, 1H), 7.68 (s, 4H), 7.62 (d, J = |
| | | | 8.2 Hz, 1H), 7.45 (d, J = 1.7 Hz, 1H), 7.31 (d, J = 8.2 Hz, 3H), |
| | | | 4.03 (d, J = 2.6 Hz, 2H), 2.82-2.71 (m, 1H), 1.23 (dd, J = 6.9, |
| | | | 3.2 Hz, 6H); |
| | | | ¹⁹F NMR (376 MHz, CDCl₃) δ −57.70 |
| J408 | | ESIMS m/z 624 | ¹H NMR (400 MHz, CDCl₃) δ 8.51 (s, 1H), 8.28-8.17 (m, 2H), |
| | | ([M + H]⁺) | 7.67 (d, J = 1.5 Hz, 4H), 7.43 (s, 1H), 7.32 (dd, J = 14.2, 8.6 |
| | | | Hz, 3H), 6.90 (dd, J = 8.8, 2.7 Hz, 1H), 6.37 (d, J = 2.7 Hz, 1H), |
| | | | 3.99 (d, J = 2.6 Hz, 2H), 2.97 (s, 6H), 2.60 (dt, J = 13.5, 6.8 Hz, |
| | | | 1H), 1.18 (dd, J = 6.9, 5.5 Hz, 6H); |
| | | | ¹⁹F NMR (376 MHz, CDCl₃) δ −57.70 |
| J409 | | ESIMS m/z 624 | ¹H NMR (400 MHz, CDCl₃) δ 8.51 (s, 1H), 8.22 (d, J = 8.8 Hz, |
| | | ([M + H]⁺) | 2H), 7.67 (d, J = 2.0 Hz, 4H), 7.42 (s, 1H), 7.31 (d, J = 8.4 Hz, |
| | | | 3H), 6.84 (dd, J = 8.7, 2.7 Hz, 1H), 6.41 (d, J = 2.7 Hz, 1H), |
| | | | 3.99 (d, J = 2.4 Hz, 2H), 2.97 (s, 6H), 2.40-2.24 (m, 2H), 1.63- |
| | | | 1.46 (m, 2H), 0.91 (t, J = 7.3 Hz, 3H); |
| | | | ¹⁹F NMR (376 MHz, CDCl₃) δ −57.71 |
| J410 | | ESIMS m/z 642 | ¹H NMR (400 MHz, CDCl₃) δ 8.52 (s, 1H), 8.22 (d, J = 8.8 Hz, |
| | | ([M + H]⁺) | 2H), 7.68 (d, J = 1.4 Hz, 4H), 7.37 (s, 1H), 7.31 (d, J = 8.3 Hz, |
| | | | 2H), 7.09 (d, J = 14.1 Hz, 1H), 6.53 (d, J = 8.3 Hz, 1H), 3.99 (d, |
| | | | J = 2.3 Hz, 2H), 2.87 (s, 6H), 2.59 (s, 1H), 1.17 (dd, J = 6.9, 3.9 |
| | | | Hz, 6H); |
| | | | ¹⁹F NMR (376 MHz, CDCl₃) δ −57.70, −119.17 |
| J411 | | ESIMS m/z 738 | ¹H NMR (500 MHz, CDCl₃) δ 8.76 (s, 1H), 8.64 (d, J = 9.0 Hz, |
| | | ([M + H]⁺) | 1H), 8.40 (s, 1H), 8.22 (d, J = 8.6 Hz, 3H), 8.11 (d, J = 8.8 Hz, |
| | | | 2H), 7.71 (s, 1H), 7.40 (d, J = 7.9 Hz, 1H), 7.36-7.32 (m, 1H), |
| | | | 7.04 (s, 1H), 4.63 (d, J = 12.5 Hz, 1H), 4.51 (d, J = 12.5 Hz, |
| | | | 1H), 3.97 (d, J = 4.7 Hz, 2H), 3.71 (dt, J = 16.5, 8.7 Hz, 2H), |
| | | | 2.46 (s, 3H); |
| | | | ¹⁹F NMR (471 MHz, CDCl₃) δ −73.75, −78.09 |
| J412 | | ESIMS m/z 749 | ¹H NMR (500 MHz, CDCl₃) δ 8.76 (s, 1H), 8.64 (d, J = 8.8 Hz, |
| | | ([M + H]⁺) | 1H), 8.41 (d, J = 9.8 Hz, 2H), 8.23 (d, J = 8.7 Hz, 2H), 8.14- |
| | | | 8.07 (m, 2H), 7.84 (dd, J = 8.7, 2.1 Hz, 1H), 7.72 (s, 1H), 7.55 |
| | | | (d, J = 2.1 Hz, 1H), 7.16 (d, J = 8.7 Hz, 1H), 4.41-4.26 (m, |
| | | | 2H), 3.98 (s, 2H), 2.62 (dt, J = 10.5, 5.3 Hz, 2H); |
| | | | ¹⁹F NMR (471 MHz, CDCl₃) δ −64.51, −78.09 |
| J413 | | ESIMS m/z 679 | ¹H NMR (500 MHz, CDCl₃) δ 8.76 (s, 1H), 8.62 (d, J = 8.7 Hz, |
| | | ([M + H]⁺) | 1H), 8.41 (d, J = 9.5 Hz, 2H), 8.23 (d, J = 8.7 Hz, 2H), 8.11 (d, |
| | | | J = 8.8 Hz, 2H), 7.82 (dd, J = 8.2, 1.7 Hz, 1H), 7.73 (s, 1H), |
| | | | 7.64 (d, J = 8.3 Hz, 1H), 7.43 (d, J = 1.7 Hz, 1H), 4.05 (s, 2H), |
| | | | 2.79-2.70 (m, 1H), 1.27 (d, J = 6.9 Hz, 3H), 1.22 (d, J = 6.9 |
| | | | Hz, 3H); |
| | | | ¹⁹F NMR (471 MHz, CDCl₃) δ −78.08 |
| J414 | | ESIMS m/z 697 | ¹H NMR (500 MHz, CDCl₃) δ 8.75 (s, 1H), 8.61 (d, J = 8.9 Hz, |
| | | ([M + H]⁺) | 1H), 8.40 (s, 2H), 8.22 (d, J = 8.6 Hz, 2H), 8.10 (d, J = 8.9 Hz, |
| | | | 2H), 7.79 (s, 1H), 7.32 (d, J = 8.8 Hz, 1H), 6.89 (dd, J = 8.8, |
| | | | 2.7 Hz, 1H), 6.33 (d, J = 2.8 Hz, 1H), 4.01 (d, J = 1.4 Hz, 2H), |
| | | | 2.97 (s, 6H), 2.56 (p, J = 6.9 Hz, 1H), 1.21 (d, J = 6.7 Hz, 3H), |
| | | | 1.16 (d, J = 6.8 Hz, 3H); |
| | | | ¹⁹F NMR (471 MHz, CDCl₃) δ −78.09 |
| J415 | | HRMS-ESI (m/z) | ¹H NMR (400 MHz, CDCl₃) δ 11.45 (s, 1H), 9.25 (s, 1H), 8.86 |
| | | [M + H]⁺ calcd for | (d, J = 8.6 Hz, 1H), 8.78-8.68 (m, 2H), 8.36 (d, J = 8.5 Hz, |
| | | C₃₀H₂₆F₃N₇O₃S, | 1H), 8.15 (d, J = 1.9 Hz, 2H), 7.39 (d, J = 8.1 Hz, 1H), 7.30 (d, |
| | | 622.1843; found, | J = 8.3 Hz, 1H), 6.89 (s, 1H), 3.99 (s, 2H), 2.72 (s, 3H), 2.71- |
| | | 622.184 | 2.66 (m, 1H), 2.38 (s, 3H), 1.22 (dd, J = 38.4, 6.9 Hz, 6H); |
| | | | ¹⁹F NMR (376 MHz, CDCl₃) δ −62.10 |
| J416 | | HRMS-ESI (m/z) | ¹H NMR (400 MHz, CDCl₃) δ 11.46 (d, J = 16.4 Hz, 1H), 9.25 |
| | | [M + H]⁺ calcd for | (s, 1H), 8.87 (s, 1H), 8.79-8.64 (m, 1H), 8.37 (d, J = 9.0 Hz, |
| | | C₃₀H₂₆F₃N₇O₄S, | 1H), 8.15 (s, 2H), 7.59-7.49 (m, 1H), 7.37 (d, J = 8.1 Hz, 1H), |
| | | 638.1792; found, | 6.92 (d, J = 4.7 Hz, 1H), 4.18 (dd, J = 25.4, 6.5 Hz, 1H), 3.99 |
| | | 638.1788 | (d, J = 2.9 Hz, 2H), 3.79-3.71 (m, 1H), 3.17 (d, J = 54.1 Hz, |
| | | | 3H), 2.71 (d, J = 10.5 Hz, 3H), 2.41 (s, 3H), 1.40 (dd, J = 49.6, |
| | | | 6.5 Hz, 3H); |
| | | | ¹⁹F NMR (376 MHz, CDCl₃) δ −62.10 |
| J417 | | HRMS-ESI (m/z) | ¹H NMR (400 MHz, CDCl₃) δ 11.44 (s, 1H), 9.25 (s, 1H), 8.86 |
| | | [M + H]⁺ calcd for | (s, 1H), 8.75 (s, 1H), 8.71 (d, J = 2.0 Hz, 1H), 8.36 (s, 1H), 8.15 |
| | | C₃₁H₂₉F₃N₈O₃S, | (d, J = 1.8 Hz, 2H), 7.34 (d, J = 8.8 Hz, 1H), 6.88 (dd, J = 8.9, |
| | | 651.2108; found, | 2.7 Hz, 1H), 6.35 (d, J = 2.8 Hz, 1H), 3.99 (s, 2H), 2.95 (s, 6H), |
| | | 651.2117 | 2.72 (s, 3H), 2.60 (p, J = 6.8 Hz, 1H), 1.20 (dd, J = 38.4, 6.9 |
| | | | Hz, 6H); |
| | | | ¹⁹F NMR (376 MHz, CDCl₃) δ −62.10 |
| J418 | | HRMS-ESI (m/z) | ¹H NMR (400 MHz, CDCl₃) δ 11.45 (s, 1H), 9.25 (s, 1H), 8.85 |
| | | [M + H]⁺ calcd for | (d, J = 8.8 Hz, 1H), 8.75 (s, 1H), 8.71 (d, J = 2.0 Hz, 1H), 8.37 |
| | | C₃₁H₂₉F₃N₈O₃S, | (d, J = 9.2 Hz, 1H), 8.15 (s, 2H), 7.42 (d, J = 7.8 Hz, 1H), 7.34 |
| | | 651.2108; found, | (d, J = 7.8 Hz, 1H), 7.04 (s, 1H), 4.67 (d, J = 12.7 Hz, 1H), 4.53 |
| | | 651.2101 | (d, J = 12.7 Hz, 1H), 3.96 (d, J = 1.4 Hz, 2H), 3.78-3.63 (m, |
| | | | 2H), 2.72 (s, 3H), 2.44 (s, 3H); |
| | | | ¹⁹F NMR (376 MHz, CDCl₃) δ −62.10, −73.79 |
| J419 | | HRMS-ESI (m/z) | ¹H NMR (400 MHz, CDCl₃) δ 11.41 (s, 1H), 9.25 (s, 1H), 8.86 |
| | | [M + H]⁺ calcd for | (s, 1H), 8.75 (s, 1H), 8.71 (d, J = 2.0 Hz, 1H), 8.36 (s, 1H), 8.15 |
| | | C₃₁H₂₉F₃N₈O₃S, | (s, 2H), 7.24 (s, 1H), 6.82 (dd, J = 8.6, 2.7 Hz, 1H), 6.40 (d, J = |
| | | 651.2108; found, | 2.7 Hz, 1H), 3.98 (d, J = 1.1 Hz, 2H), 2.96 (s, 6H), 2.73 (s, 3H), |
| | | 651.2113 | 2.37-2.30 (m, 2H), 1.61-1.50 (m, 2H), 0.90 (t, J = 7.3 Hz, |
| | | | 3H); |
| | | | ¹⁹F NMR (376 MHz, CDCl₃) δ −62.10 |
| J420 | | HRMS-ESI (m/z) | ¹H NMR (400 MHz, CDCl₃) δ 8.53 (s, 1H), 8.18 (d, J = 8.5 Hz, |
| | | [M + H]⁺ calcd for | 1H), 8.09-7.91 (m, 2H), 7.84-7.74 (m, 2H), 7.45-7.30 (m, |
| | | C₃₀H₂₅F₅N₆O₄S, | 4H), 7.05 (s, 2H), 5.81 (tt, J = 55.4, 4.1 Hz, 1H), 4.56 (d, J = |
| | | 661.1651; found, | 12.4 Hz, 1H), 4.45 (d, J = 12.4 Hz, 1H), 4.03-3.85 (m, 2H), |
| | | 661.1659 | 3.58 (td, J = 13.9, 4.1 Hz, 2H), 2.44 (s, 3H), 2.26 (s, 3H); |
| | | | ¹⁹F NMR (376 MHz, CDCl₃) δ −58.03, −124.85 |
| J421 | | ESIMS m/z 643 | ¹H NMR (400 MHz, CDCl₃) δ 8.52 (d, J = 10.2 Hz, 1H), 8.46 (t, |
| | | ([M + H]⁺) | J = 7.3 Hz, 1H), 7.97 (d, J = 8.6 Hz, 1H), 7.92-7.83 (m, 1H), |
| | | | 7.83-7.70 (m, 2H), 7.55-7.44 (m, 1H), 7.38 (d, J = 9.3 Hz, |
| | | | 3H), 6.90 (d, J = 23.4 Hz, 2H), 4.03-3.84 (m, 3H), 3.17 (d, J = |
| | | | 22.7 Hz, 3H), 2.43 (s, 3H), 1.73-1.57 (m, 2H), 0.92 (dt, J = |
| | | | 11.0, 7.3 Hz, 3H); |
| | | | ¹⁹F NMR (376 MHz, CDCl₃) δ −58.03, −131.26, −135.37 |
| J422 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 7.98 (d, J = 8.6 Hz, 1H), 7.94-7.82 (m, 5H), 7.53-7.31 |
| | | C₂₈H₂₁F₉N₆O₃S2, | (m, 3H), 7.09-6.94 (m, 1H), 4.63 (d, J = 12.4 Hz, 1H), 4.50 (d, |
| | | 725.1046; found, | J = 12.4 Hz, 1H), 3.96 (d, J = 2.7 Hz, 2H), 3.71 (qd, J = 8.7, 3.9 |
| | | 725.1052 | Hz, 2H), 2.45 (s, 3H); |
| | | | ¹⁹F NMR (376 MHz, CDCl₃) δ −73.79, −131.25 |
| J423 | | HRMS-ESI (m/z) | ¹H NMR (400 MHz, CDCl₃) δ 8.63 (s, 1H), 8.48 (td, J = 8.3, 4.7 |
| | | [M + H]⁺ calcd for | Hz, 1H), 8.02-7.92 (m, 2H), 7.92-7.80 (m, 3H), 7.53 (dt, J = |
| | | C₂₈H₂₄F₆N₆O₃S2 | 10.8, 5.5 Hz, 2H), 7.45-7.32 (m, 2H), 6.93 (s, 1H), 4.21- |
| | | 671.1328; found, | 4.09 (m, 1H), 4.00 (dd, J = 3.0, 1.9 Hz, 2H), 3.15 (d, J = 22.2 |
| | | 671.1331 | Hz, 3H), 2.43 (s, 3H), 1.37 (dd, J = 16.2, 6.4 Hz, 3H); |
| | | | ¹⁹F NMR (376 MHz, CDCl₃) δ −131.00, −131.44 |
| J425 | | HRMS-ESI (m/z) | ¹H NMR (400 MHz, CDCl₃) δ 8.56 (s, 1H), 8.48 (t, J = 8.4 Hz, |
| | | [M + H]⁺ calcd for | 1H), 7.97 (d, J = 8.6 Hz, 1H), 7.89 (d, J = 12.7 Hz, 1H), 7.78 (d, |
| | | C₃₀H₂₃F₄N₇O₃S, | J = 8.6 Hz, 2H), 7.50 (d, J = 8.4 Hz, 2H), 7.47 (s, 2H), 7.42 (d, |
| | | 638.1592; found, | J = 7.8 Hz, 1H), 7.35 (d, J = 7.8 Hz, 1H), 7.04 (s, 1H), 4.63 (d, |
| | | 638.1592 | J = 12.4 Hz, 1H), 4.50 (d, J = 12.4 Hz, 1H), 3.96 (d, J = 2.8 Hz, |
| | | | 2H), 3.84 (s, 2H), 3.71 (qd, J = 8.6, 3.9 Hz, 2H), 2.45 (s, 3H); |
| | | | ¹⁹F NMR (376 MHz, CDCl₃) δ −73.78, −131.41 |
| J426 | | ESIMS m/z 630 | ¹H NMR (400 MHz, CDCl₃) δ 8.59-8.49 (m, 1H), 8.26 (d, J = |
| | | ([M + H]⁺) | 8.8 Hz, 2H), 7.96 (d, J = 8.7 Hz, 1H), 7.86 (d, J = 11.5 Hz, 1H), |
| | | | 7.58-7.46 (m, 2H), 7.39 (d, J = 8.2 Hz, 3H), 6.93 (s, 1H), 4.21- |
| | | | 4.09 (m, 1H), 4.00 (d, J = 3.7 Hz, 2H), 3.15 (d, J = 21.4 Hz, |
| | | | 3H), 2.43 (s, 3H), 1.37 (dd, J = 15.0, 6.5 Hz, 3H); |
| | | | ¹⁹F NMR (376 MHz, CDCl₃) δ −57.64, −130.45, −130.88 |
| J427 | | ESIMS m/z 684 | ¹H NMR (400 MHz, CDCl₃) δ 8.62 (t, J = 8.3 Hz, 1H), 8.23- |
| | | ([M + H]⁺) | 8.17 (m, 2H), 8.00 (d, J = 8.9 Hz, 1H), 7.90 (d, J = 11.4 Hz, |
| | | | 1H), 7.58 (s, 1H), 7.42 (d, J = 7.8 Hz, 1H), 7.35 (d, J = 8.6 Hz, |
| | | | 3H), 7.04 (s, 1H), 4.62 (d, J = 12.4 Hz, 1H), 4.50 (d, J = 12.4 |
| | | | Hz, 1H), 3.98 (d, J = 2.7 Hz, 2H), 3.77-3.66 (m, 2H), 2.45 (s, |
| | | | 3H); |
| | | | ¹⁹F NMR (376 MHz, CDCl₃) δ −57.68, −73.79, −130.07 |
| J428 | | ESIMS m/z 682 | ¹H NMR (400 MHz, CDCl₃) δ 8.59 (t, J = 8.1 Hz, 1H), 8.20 (d, J = |
| | | ([M − H]⁻) | 8.8 Hz, 2H), 8.01 (d, J = 8.5 Hz, 1H), 7.90 (d, J = 10.8 Hz, |
| | | | 1H), 7.68 (d, J = 8.1 Hz, 1H), 7.50-7.41 (m, 2H), 7.35 (d, J = |
| | | | 8.3 Hz, 2H), 7.02 (s, 1H), 4.49-4.41 (m, 1H), 4.01 (d, J = 9.3 |
| | | | Hz, 2H), 3.43 (s, 3H), 2.47 (s, 3H); |
| | | | ¹⁹F NMR (376 MHz, CDCl₃) δ −57.68, −76.31, −130.62 |
| J429 | | ESIMS m/z 630 | ¹H NMR (500 MHz, CDCl₃) δ 8.59 (s, 1H), 8.18 (dd, J = 8.3, 6.0 |
| | | ([M + H]⁺) | Hz, 2H), 7.87 (dd, J = 24.0, 9.4 Hz, 2H), 7.63-7.51 (m, 2H), |
| | | | 7.43-7.36 (m, 3H), 6.93 (s, 1H), 4.18-4.12 (m, 1H), 4.01 (d, |
| | | | J = 6.2 Hz, 1H), 3.16 (dd, J = 25.6, 5.9 Hz, 3H), 2.44 (d, J = 5.4 |
| | | | Hz, 3H), 1.41-1.32 (m, 3H); |
| | | | ¹⁹F NMR (471 MHz, CDCl₃) δ −57.65, −129.73, −130.15 |
| J430 | | ESIMS m/z 684 | ¹H NMR (500 MHz, CDCl₃) δ 8.60 (t, J = 8.2 Hz, 1H), 8.18 (d, J = |
| | | ([M + H]⁺) | 8.5 Hz, 2H), 7.89 (d, J = 8.7 Hz, 1H), 7.85 (d, J = 11.3 Hz, |
| | | | 1H), 7.55 (s, 1H), 7.38 (dq, J = 14.3, 7.8 Hz, 4H), 7.04 (s, 1H), |
| | | | 4.62 (d, J = 12.4 Hz, 1H), 4.50 (d, J = 12.3 Hz, 1H), 3.97 (d, J = |
| | | | 4.1 Hz, 2H), 3.70 (td, J = 8.8, 4.9 Hz, 2H), 2.45 (s, 3H); |
| | | | ¹⁹F NMR (471 MHz, CDCl₃) δ −57.66, −73.78, −130.00 |
| J431 | | ESIMS m/z 643 | ¹H NMR (500 MHz, CDCl₃) δ 8.61 (t, J = 8.2 Hz, 1H), 8.18 (d, J = |
| | | ([M + H]⁺) | 8.7 Hz, 2H), 7.89 (d, J = 8.7 Hz, 1H), 7.84 (d, J = 11.3 Hz, |
| | | | 1H), 7.67 (s, 1H), 7.38 (d, J = 8.5 Hz, 2H), 7.34 (d, J = 8.8 Hz, |
| | | | 1H), 6.93-6.87 (m, 1H), 6.35 (d, J = 2.7 Hz, 1H), 4.00 (d, J = |
| | | | 2.7 Hz, 2H), 2.97 (s, 6H), 2.57 (p, J = 6.7 Hz, 1H), 1.17 (dd, J = |
| | | | 9.8, 6.9 Hz, 6H); |
| | | | ¹⁹F NMR (471 MHz, CDCl₃) δ −57.66, −129.35 |
| J432 | | ESIMS m/z 661 | ¹H NMR (500 MHz, CDCl₃) δ 8.60 (t, J = 8.2 Hz, 1H), 8.21- |
| | | ([M + H]⁺) | 8.15 (m, 2H), 7.90 (d, J = 8.7 Hz, 1H), 7.85 (d, J = 11.0 Hz, |
| | | | 1H), 7.62 (s, 1H), 7.38 (d, J = 8.5 Hz, 2H), 7.09 (d, J = 14.1 Hz, |
| | | | 1H), 6.52 (d, J = 8.3 Hz, 1H), 4.00 (d, J = 2.6 Hz, 2H), 2.87 (s, |
| | | | 6H), 2.57 (p, J = 7.2 Hz, 1H), 1.17 (t, J = 6.7 Hz, 6H); |
| | | | ¹⁹F NMR (471 MHz, CDCl₃) δ −57.66, −119.18, −129.66 |
| J433 | | ESIMS m/z 684 | ¹H NMR (500 MHz, CDCl₃) δ 8.56 (t, J = 8.3 Hz, 1H), 8.23- |
| | | ([M + H]⁺) | 8.15 (m, 2H), 7.90 (d, J = 8.7 Hz, 1H), 7.85 (d, J = 11.2 Hz, |
| | | | 1H), 7.68 (d, J = 8.0 Hz, 1H), 7.41 (dd, J = 29.3, 8.4 Hz, 4H), |
| | | | 7.02 (s, 1H), 4.46 (q, J = 6.3 Hz, 1H), 4.01 (d, J = 13.2 Hz, 2H), |
| | | | 3.43 (s, 3H), 2.47 (s, 3H); |
| | | | ¹⁹F NMR (471 MHz, CDCl₃) δ −57.66, −76.32, −130.55 |
| J434 | | ESIMS m/z 630 | ¹H NMR (400 MHz, CDCl₃) δ 8.66-8.54 (m, 1H), 8.19 (d, J = |
| | | ([M + H]⁺) | 8.4 Hz, 2H), 8.00 (d, J = 8.7 Hz, 1H), 7.89 (d, J = 11.2 Hz, 1H), |
| | | | 7.61 (d, J = 22.1 Hz, 1H), 7.53 (t, J = 6.8 Hz, 1H), 7.37 (dd, J = |
| | | | 22.4, 7.0 Hz, 3H), 6.93 (s, 1H), 4.15 (t, J = 6.2 Hz, 1H), 4.00 (d, |
| | | | J = 4.6 Hz, 2H), 3.15 (d, J = 20.7 Hz, 3H), 2.43 (s, 3H), 1.37 |
| | | | (dd, J = 13.1, 6.4 Hz, 3H); |
| | | | ¹⁹F NMR (376 MHz, CDCl₃) δ −57.68, −129.75, −130.16 |
| J435 | | ESIMS m/z 643 | ¹H NMR (400 MHz, CDCl₃) δ 8.63 (t, J = 8.2 Hz, 1H), 8.26- |
| | | ([M + H]⁺) | 8.13 (m, 2H), 8.00 (d, J = 8.7 Hz, 1H), 7.89 (d, J = 11.2 Hz, |
| | | | 1H), 7.73 (s, 1H), 7.37-7.32 (m, 3H), 6.91 (dd, J = 6.8, 4.5 |
| | | | Hz, 1H), 6.36 (t, J = 2.2 Hz, 1H), 4.00 (d, J = 1.9 Hz, 2H), 2.97 |
| | | | (d, J = 1.8 Hz, 6H), 2.64-2.52 (m, 1H), 1.20-1.13 (m, 6H); |
| | | | ¹⁹F NMR (376 MHz, CDCl₃) δ −57.67, −129.39 |
| J436 | | HRMS-ESI (m/z) | ¹H NMR (400 MHz, CDCl₃) δ 8.53 (s, 1H), 8.18 (d, J = 8.5 Hz, |
| | | [M + H]⁺ calcd for | 1H), 8.03 (d, J = 8.7 Hz, 1H), 7.98 (s, 1H), 7.86-7.69 (m, 2H), |
| | | C₃₀H₂₄ClF₅N₆O₄S, | 7.44 (d, J = 7.8 Hz, 1H), 7.36 (dd, J = 15.8, 8.2 Hz, 3H), 7.05 |
| | | 695.1261; found, | (s, 2H), 4.65 (d, J = 12.4 Hz, 1H), 4.55 (d, J = 12.4 Hz, 1H), |
| | | 695.1253 | 3.94 (d, J = 3.0 Hz, 2H), 3.83 (t, J = 11.2 Hz, 2H), 2.45 (s, 3H), |
| | | | 2.26 (s, 3H); |
| | | | ¹⁹F NMR (376 MHz, CDCl₃) δ −58.03, −61.23 |
| J437 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (d, J = 8.6 Hz, 1H), 7.88 (dd, J = 12.0, 1.8 Hz, 1H), |
| | | C₂₉H₂₁ClF₆N₆O₄S, | 7.84-7.72 (m, 2H), 7.52-7.31 (m, 5H), 7.04 (s, 1H), 4.65 (d, |
| | | 699.101; found, | J = 12.4 Hz, 1H), 4.54 (d, J = 12.4 Hz, 1H), 3.96 (d, J = 3.8 Hz, |
| | | 699.1 | 2H), 3.81 (td, J = 11.3, 2.6 Hz, 2H), 2.45 (s, 3H); |
| | | | ¹⁹F NMR (376 MHz, CDCl₃) δ −58.03, −61.30, −131.33 |
| J438 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (d, J = 8.4 Hz, 1H), 7.88 (dd, J = 12.0, 1.8 Hz, 1H), |
| | | C₂₉H₂₁F₇N₆O₄S, | 7.83-7.71 (m, 2H), 7.46 (d, J = 3.1 Hz, 1H), 7.36 (dd, J = |
| | | 683.1306; found, | 18.4, 6.9 Hz, 4H), 7.11 (d, J = 8.5 Hz, 1H), 4.62 (d, J = 12.5 |
| | | 683.1292 | Hz, 1H), 4.51 (d, J = 12.4 Hz, 1H), 3.95 (d, J = 1.9 Hz, 2H), |
| | | | 3.74 (qd, J = 8.6, 2.4 Hz, 2H), 2.47 (s, 3H); |
| | | | ¹⁹F NMR (376 MHz, CDCl₃) δ −58.03, −73.79, −131.41 |
| J439 | | HRMS-ESI (m/z) | ¹H NMR (400 MHz, CDCl₃) δ 8.54 (s, 1H), 8.46 (t, J = 8.4 Hz, |
| | | [M + H]⁺ calcd for | 1H), 7.97 (d, J = 8.8 Hz, 1H), 7.90 (dd, J = 12.0, 1.8 Hz, 1H), |
| | | C₃₄H₂₃F₇N₆O₃S, | 7.84-7.77 (m, 2H), 7.46 (d, J = 3.0 Hz, 1H), 7.45-7.31 (m, |
| | | 729.1513; found, | 6H), 7.30-7.27 (m, 2H), 6.96 (d, J = 1.6 Hz, 1H), 3.90 (s, 2H), |
| | | 729.1505 | 3.80 (d, J = 18.1 Hz, 1H), 3.59 (d, J = 18.1 Hz, 1H), 2.42 (s, |
| | | | 3H); |
| | | | ¹⁹F NMR (376 MHz, CDCl₃) δ −58.02, −62.59, −131.25 |
| J440 | | HRMS-ESI (m/z) | ¹H NMR (400 MHz, CDCl₃) δ 8.54 (s, 1H), 8.18 (d, J = 8.5 Hz, |
| | | [M + H]⁺ calcd for | 1H), 8.04 (d, J = 8.5 Hz, 1H), 8.00 (s, 1H), 7.82-7.77 (m, 2H), |
| | | C₃₅H₂₆F₆N₆O₃S, | 7.44 (d, J = 7.8 Hz, 1H), 7.40-7.33 (m, 4H), 7.32-7.22 (m, |
| | | 725.1764; found, | 3H), 7.09 (s, 1H), 6.98 (s, 1H), 3.91 (s, 2H), 3.78 (d, J = 18.3 |
| | | 725.1753 | Hz, 1H), 3.57 (d, J = 18.1 Hz, 1H), 2.42 (s, 3H), 2.28 (s, 3H); |
| | | | ¹⁹F NMR (376 MHz, CDCl₃) δ −58.03, −62.52 |
| J441 | | ESIMS m/z 679 | ¹H NMR (500 MHz, CDCl₃) δ 8.51 (s, 1H), 8.21 (dd, J = 9.0, 7.3 |
| | | ([M + H]⁺) | Hz, 3H), 7.56 (d, J = 2.5 Hz, 1H), 7.53 (dd, J = 8.8, 2.6 Hz, 1H), |
| | | | 7.33-7.27 (m, 3H), 7.07-7.02 (m, 2H), 6.97 (d, J = 8.5 Hz, |
| | | | 1H), 4.23 (td, J = 6.3, 2.7 Hz, 2H), 3.93 (d, J = 2.5 Hz, 2H), |
| | | | 2.56 (dtd, J = 16.7, 10.6, 6.2 Hz, 2H), 2.38 (s, 3H), 2.30 (s, 3H); |
| | | | ¹⁹F NMR (376 MHz, CDCl₃) δ −57.71, −64.57 |
| J442 | | ESIMS m/z 679 | ¹H NMR (500 MHz, CDCl₃) δ 8.51 (s, 1H), 8.23-8.20 (m, 2H), |
| | | ([M + H]⁺) | 8.19 (d, J = 8.7 Hz, 1H), 7.59-7.50 (m, 2H), 7.43 (d, J = 7.8 |
| | | | Hz, 1H), 7.34 (d, J = 7.8 Hz, 1H), 7.31 (d, J = 8.3 Hz, 2H), 7.04 |
| | | | (d, J = 17.9 Hz, 2H), 4.63 (d, J = 12.4 Hz, 1H), 4.52 (d, J = 12.4 |
| | | | Hz, 1H), 3.95 (d, J = 3.1 Hz, 2H), 3.73 (q, J = 8.7 Hz, 2H), 2.45 |
| | | | (s, 3H), 2.28 (s, 3H) |
| J443 | | ESIMS m/z 679 | ¹H NMR (500 MHz, CDCl₃) δ 8.51 (s, 1H), 8.23-8.20 (m, 2H), |
| | | ([M + H]⁺) | 8.18 (d, J = 8.7 Hz, 1H), 7.67 (d, J = 8.1 Hz, 1H), 7.57 (d, J = |
| | | | 2.5 Hz, 1H), 7.56-7.52 (m, 1H), 7.43 (dd, J = 8.1, 1.7 Hz, 1H), |
| | | | 7.33-7.29 (m, 2H), 7.05-7.02 (m, 1H), 6.94 (s, 1H), 4.50 (q, |
| | | | J = 6.2 Hz, 1H), 4.07-3.91 (m, 2H), 3.45 (s, 3H), 2.46 (s, 3H), |
| | | | 2.22 (s, 3H); |
| | | | ¹⁹F NMR (376 MHz, CDCl₃) δ −57.71, −76.32 |
| J444 | | ESIMS m/z 638 | ¹H NMR (500 MHz, CDCl₃) δ 8.51 (s, 1H), 8.25-8.18 (m, 3H), |
| | | ([M − H]⁻) | 7.56 (d, J = 2.6 Hz, 1H), 7.54-7.50 (m, 1H), 7.33 (d, J = 8.6 |
| | | | Hz, 1H), 7.31 (d, J = 8.4 Hz, 2H), 7.10 (s, 1H), 6.80 (dd, J = |
| | | | 8.6, 2.7 Hz, 1H), 6.49 (d, J = 2.6 Hz, 1H), 4.35 (d, J = 12.2 Hz, |
| | | | 1H), 4.21 (d, J = 12.2 Hz, 1H), 4.00-3.88 (m, 2H), 3.24 (s, |
| | | | 3H), 3.01 (s, 6H), 2.29 (s, 3H); |
| | | | ¹⁹F NMR (376 MHz, CDCl₃) δ −57.71 |
| J445 | | HRMS-ESI (m/z) | ¹H NMR (400 MHz, CDCl₃) δ 8.63 (s, 1H), 8.50 (t, J = 8.4 Hz, |
| | | [M + H]⁺ calcd for | 1H), 8.02-7.93 (m, 2H), 7.93-7.80 (m, 4H), 7.50 (s, 1H), |
| | | C₂₈H₂₄F₆N₆O₃S2, | 7.40 (d, J = 7.8 Hz, 1H), 7.30 (d, J = 7.9 Hz, 1H), 7.01 (s, 1H), |
| | | 671.1328; found, | 4.50-4.29 (m, 2H), 3.95 (s, 2H), 3.41 (q, J = 6.9 Hz, 2H), 2.43 |
| | | 671.1314 | (s, 3H), 1.15 (t, J = 7.0 Hz, 3H) |
| J446 | | HRMS-ESI (m/z) | ¹H NMR (400 MHz, CDCl₃) δ 8.63 (s, 1H), 8.49 (t, J = 8.4 Hz, |
| | | [M + H]⁺ calcd for | 1H), 8.03-7.95 (m, 1H), 7.95-7.82 (m, 4H), 7.49 (d, J = 3.1 |
| | | C₂₈H₂₂F₈N₆O₃S2, | Hz, 1H), 7.43-7.29 (m, 3H), 7.03 (d, J = 1.8 Hz, 1H), 5.80 (tt, |
| | | 707.114; found, | J = 55.4, 4.1 Hz, 1H), 4.56 (d, J = 12.4 Hz, 1H), 4.43 (dd, J = |
| | | 707.1132 | 12.3, 5.3 Hz, 1H), 4.04-3.86 (m, 2H), 3.57 (td, J = 14.0, 4.1 |
| | | | Hz, 3H), 2.42 (d, J = 20.0 Hz, 3H); |
| | | | ¹⁹F NMR (376 MHz, CDCl₃) δ −124.91, −124.96, −131.18 |
| J447 | | HRMS-ESI (m/z) | ¹H NMR (400 MHz, CDCl₃) δ 8.63 (s, 1H), 8.51 (t, J = 8.3 Hz, |
| | | [M + H]⁺ calcd for | 1H), 7.98 (d, J = 8.6 Hz, 1H), 7.94-7.82 (m, 4H), 7.51 (d, J = |
| | | C₂₈H₂₁F₉N₆O₃S2, | 3.0 Hz, 1H), 7.09-6.91 (m, 3H), 4.27-4.14 (m, 2H), 3.93 (d, |
| | | 725.1046; found, | J = 1.6 Hz, 2H), 2.62-2.47 (m, 3H), 2.38 (s, 3H); |
| | | 725.1035 | ¹⁹F NMR (376 MHz, CDCl₃) δ −64.49, −131.32, −131.37 |
| J448 | | HRMS-ESI (m/z) | ¹H NMR (400 MHz, CDCl₃) δ 8.53 (s, 1H), 8.18 (d, J = 8.5 Hz, |
| | | [M + H]⁺ calcd for | 1H), 8.09-8.00 (m, 1H), 7.98 (s, 1H), 7.84-7.72 (m, 2H), |
| | | C₃₀H₂₄F₆N₆O₄S, | 7.36 (dd, J = 16.5, 10.0 Hz, 4H), 7.12 (d, J = 7.9 Hz, 1H), 7.04 |
| | | 679.1557; found, | (s, 1H), 4.62 (d, J = 12.5 Hz, 1H), 4.52 (d, J = 12.5 Hz, 1H), |
| | | 679.155 | 3.94 (d, J = 1.6 Hz, 2H), 3.76 (q, J = 8.7 Hz, 2H), 2.46 (s, 3H), |
| | | | 2.25 (s, 3H); |
| | | | ¹⁹F NMR (376 MHz, CDCl₃) δ −58.03, −73.76 |
| J449 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 7.98 (d, J = 8.6 Hz, 1H), 7.95-7.83 (m, 5H), 7.51-7.32 |
| | | C₂₈H₂₁ClF₈N₆O₃S2, | (m, 3H), 7.10-6.96 (m, 1H), 4.65 (d, J = 12.4 Hz, 1H), 4.54 (d, |
| | | 741.075; found, | J = 12.4 Hz, 1H), 3.96 (d, J = 3.7 Hz, 2H), 3.81 (td, J = 11.3, |
| | | 741.0738 | 2.5 Hz, 2H), 2.45 (s, 3H); |
| | | | ¹⁹F NMR (376 MHz, CDCl₃) δ −61.31, −131.20 |
| J450 | | HRMS-ESI (m/z) | ¹H NMR (400 MHz, CDCl₃) δ 8.63 (s, 1H), 8.50 (t, J = 8.3 Hz, |
| | | [M + H]⁺ calcd for | 1H), 7.97 (d, J = 8.6 Hz, 1H), 7.95-7.83 (m, 5H), 7.57 (s, 1H), |
| | | C₂₉H₂₇F₆N₇O₂S2, | 7.33 (d, J = 8.8 Hz, 1H), 6.89 (dd, J = 8.9, 2.7 Hz, 1H), 6.36 (d, |
| | | 684.1645; found, | J = 2.7 Hz, 1H), 3.99 (d, J = 1.7 Hz, 2H), 2.97 (s, 6H), 2.58 (p, |
| | | 684.164 | J = 6.9 Hz, 1H), 1.17 (dd, J = 9.2, 6.8 Hz, 6H) |
| J451 | | HRMS-ESI (m/z) | ¹H NMR (400 MHz, CDCl₃) δ 9.21 (s, 1H), 8.74 (dd, J = 2.2, 1.2 |
| | | [M + H]⁺ calcd for | Hz, 1H), 8.50 (t, J = 8.3 Hz, 1H), 8.19-8.05 (m, 2H), 8.00 (d, J = |
| | | C₂₈H₂₀F₇N₇O₃S, | 8.3 Hz, 1H), 7.91 (dd, J = 12.0, 1.8 Hz, 1H), 7.48 (d, J = 2.9 |
| | | 668.1309; found, | Hz, 1H), 7.34 (d, J = 4.7 Hz, 2H), 7.11 (d, J = 8.5 Hz, 1H), 4.62 |
| | | 668.1304 | (d, J = 12.5 Hz, 1H), 4.51 (d, J = 12.4 Hz, 1H), 3.96 (d, J = 1.9 |
| | | | Hz, 2H), 3.74 (qd, J = 8.7, 2.4 Hz, 2H), 2.47 (s, 3H); |
| | | | ¹⁹F NMR (376 MHz, CDCl₃) δ −62.10, −73.79, −131.31 |
| J452 | | HRMS-ESI (m/z) | ¹H NMR (400 MHz, CDCl₃) δ 8.54 (s, 1H), 8.47 (t, J = 8.4 Hz, |
| | | [M + H]⁺ calcd for | 1H), 8.03-7.93 (m, 1H), 7.88 (dd, J = 12.0, 1.8 Hz, 1H), 7.82- |
| | | C₂₉H₂₁Cl3F₄N₆O₄S, | 7.68 (m, 2H), 7.54-7.43 (m, 2H), 7.44-7.32 (m, 3H), 7.08- |
| | | 731.0419; found, | 6.98 (m, 1H), 4.77 (d, J = 12.5 Hz, 1H), 4.68 (d, J = 12.5 Hz, |
| | | 731.0406 | 1H), 4.06-3.87 (m, 4H), 2.45 (s, 3H); |
| | | | ¹⁹F NMR (376 MHz, CDCl₃) δ −58.02, −131.23 |
| J453 | | HRMS-ESI (m/z) | ¹H NMR (400 MHz, CDCl₃) δ 8.63 (s, 1H), 8.48 (t, J = 8.3 Hz, |
| | | [M + H]⁺ calcd for | 1H), 7.98 (d, J = 8.6 Hz, 1H), 7.95-7.83 (m, 5H), 7.55-7.44 |
| | | C₂₈H₂₁Cl3F₆N₆O₃S2, | (m, 2H), 7.36 (d, J = 7.8 Hz, 1H), 7.04 (d, J = 1.7 Hz, 1H), 4.77 |
| | | 773.0159; found, | (d, J = 12.5 Hz, 1H), 4.68 (d, J = 12.5 Hz, 1H), 4.07-3.87 (m, |
| | | 773.0151 | 4H), 2.45 (s, 3H) |
| J454 | | ESIMS m/z 598 | ¹H NMR (400 MHz, CDCl₃) δ 9.22 (s, 1H), 8.80-8.69 (m, 1H), |
| | | ([M + H]⁺) | 8.51 (t, J = 8.3 Hz, 1H), 8.18-8.07 (m, 2H), 8.07-7.97 (m, |
| | | | 1H), 7.91 (dd, J = 12.0, 1.8 Hz, 1H), 7.54 (s, 1H), 7.36-7.28 |
| | | | (m, 2H), 6.93 (s, 1H), 3.99 (d, J = 2.5 Hz, 2H), 2.39 (m, 5H), |
| | | | 1.65-1.56 (m, 2H), 0.93 (t, J = 7.3 Hz, 3H); |
| | | | ¹⁹F NMR (376 MHz, CDCl₃) δ −62.10, −130.96 |
| J455 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (d, J = 8.6 Hz, 1H), 7.88 (dd, J = 12.1, 1.8 Hz, 1H), |
| | | C₃₀H₂₃F₇N₆O₄S, | 7.82-7.73 (m, 2H), 7.49 (d, J = 3.0 Hz, 1H), 7.43-7.31 (m, |
| | | 697.1462; found, | 2H), 7.28 (s, 1H), 6.99 (s, 1H), 4.60 (d, J = 12.3 Hz, 1H), 4.47 |
| | | 697.1455 | (d, J = 12.4 Hz, 1H), 3.95 (d, J = 2.4 Hz, 2H), 3.71 (qd, J = 8.8, |
| | | | 3.3 Hz, 2H), 2.35 (d, J = 10.0 Hz, 6H); |
| | | | ¹⁹F NMR (376 MHz, CDCl₃) δ −58.02, −73.77, −131.45 |
| J456 | | HRMS-ESI (m/z) | ¹H NMR (400 MHz, CDCl₃) δ 8.55 (s, 1H), 8.21 (d, J = 8.5 Hz, |
| | | [M + H]⁺ calcd for | 1H), 8.05 (d, J = 8.6 Hz, 1H), 8.00 (s, 1H), 7.85-7.77 (m, 2H), |
| | | C₃₄H₂₇F₃N₆O₃S, | 7.39 (d, J = 8.6 Hz, 2H), 7.28 (s, 4H), 7.18 (d, J = 7.2 Hz, 1H), |
| | | 657.189; found, | 7.12 (d, J = 7.5 Hz, 2H), 7.07 (s, 1H), 6.96 (s, 1H), 3.87 (s, 2H), |
| | | 657.1879 | 3.72 (d, J = 18.0 Hz, 1H), 3.49 (d, J = 18.0 Hz, 1H), 2.41 (s, |
| | | | 3H), 2.30 (s, 3H); |
| | | | ¹⁹F NMR (376 MHz, CDCl₃) δ −58.02 |
| J457 | | HRMS-ESI (m/z) | ¹H NMR (400 MHz, CDCl₃) δ 8.55 (s, 1H), 8.48 (t, J = 8.4 Hz, |
| | | [M + H]⁺ calcd for | 1H), 7.99 (s, 1H), 7.91 (dd, J = 12.0, 1.8 Hz, 1H), 7.82-7.76 |
| | | C₃₃H₂₄F₄N₆O₃S, | (m, 2H), 7.48 (d, J = 3.1 Hz, 1H), 7.39 (d, J = 8.5 Hz, 2H), 7.34- |
| | | 661.1639; found, | 7.20 (m, 4H), 7.16 (d, J = 7.2 Hz, 1H), 7.10 (d, J = 7.5 Hz, |
| | | 661.1632 | 2H), 6.94 (s, 1H), 3.86 (s, 2H), 3.74 (d, J = 18.0 Hz, 1H), 3.51 |
| | | | (d, J = 18.0 Hz, 1H), 2.41 (s, 3H); |
| | | | ¹⁹F NMR (376 MHz, CDCl₃) δ −58.01, −131.07 |
| J458 | | HRMS-ESI (m/z) | ¹H NMR (400 MHz, CDCl₃) δ 8.55 (s, 1H), 8.40 (t, J = 8.3 Hz, |
| | | [M + H]⁺ calcd for | 1H), 7.99 (d, J = 8.6 Hz, 1H), 7.91 (dd, J = 11.9, 1.8 Hz, 1H), |
| | | C₂₉H₁₉Cl2F₇N₆O₄S, | 7.83-7.72 (m, 3H), 7.64 (d, J = 3.0 Hz, 1H), 7.51-7.42 (m, |
| | | 751.0527; found, | 1H), 7.42-7.35 (m, 3H), 7.14 (d, J = 1.3 Hz, 1H), 4.66-4.53 |
| | | 751.0508 | (m, 2H), 3.74 (dt, J = 9.0, 8.3 Hz, 2H), 2.47 (s, 3H); |
| | | | ¹⁹F NMR (376 MHz, CDCl₃) δ −58.01, −73.60, −130.80 |
| J459 | | HRMS-ESI (m/z) | ¹H NMR (400 MHz, CDCl₃) δ 8.54 (s, 1H), 8.17 (d, J = 8.5 Hz, |
| | | [M + H]⁺ calcd for | 1H), 8.03 (dd, J = 8.6, 2.0 Hz, 1H), 8.00-7.91 (m, 1H), 7.87- |
| | | C₃₀H₂₄Cl3F₃N₆O₄S, | 7.72 (m, 2H), 7.50 (d, J = 7.8 Hz, 1H), 7.45-7.31 (m, 3H), |
| | | 727.067; found, | 7.12-7.00 (m, 2H), 4.77 (d, J = 12.6 Hz, 1H), 4.70 (d, J = 12.5 |
| | | 727.057 | Hz, 1H), 4.05-3.89 (m, 4H), 2.45 (s, 3H), 2.26 (s, 3H); |
| | | | ¹⁹F NMR (376 MHz, CDCl₃) δ −58.02 |
| J460 | | HRMS-ESI (m/z) | ¹H NMR (400 MHz, CDCl₃) δ 8.54 (s, 1H), 8.18 (d, J = 8.5 Hz, |
| | | [M + H]⁺ calcd for | 1H), 8.03 (dd, J = 8.4, 2.0 Hz, 1H), 7.98 (d, J = 2.0 Hz, 1H), |
| | | C₃₁H₂₆F₆N₆O₄S, | 7.85-7.72 (m, 2H), 7.43-7.33 (m, 2H), 7.29 (s, 1H), 7.03 (d, |
| | | 693.1713; found, | J = 23.0 Hz, 2H), 4.61 (d, J = 12.4 Hz, 1H), 4.48 (d, J = 12.3 |
| | | 693.1708 | Hz, 1H), 3.93 (d, J = 1.7 Hz, 2H), 3.73 (q, J = 8.7 Hz, 2H), 2.34 |
| | | | (d, J = 9.0 Hz, 6H), 2.29 (s, 3H); |
| | | | ¹⁹F NMR (376 MHz, CDCl₃) δ −58.02, −73.74 |
| J461 | | ESIMS m/z 613 | ¹H NMR (400 MHz, CDCl₃) 8.77 (s, 1H), 8.42 (d, J = 1.2 Hz, |
| | | ([M + H]⁺) | 1H), 8.08 (dd, J = 8.8, 2.2 Hz, 1H), 7.93 (d, J = 1.3 Hz, 1H), |
| | | | 7.70-7.62 (m, 1H), 7.59 (d, J = 8.1 Hz, 1H), 7.57-7.44 (m, |
| | | | 4H), 7.40 (d, J = 14.8 Hz, 1H), 6.93 (s, 1H), 4.10-4.06 (m, |
| | | | 1H), 3.99 (dd, J = 2.9, 1.7 Hz, 2H), 3.21-3.08 (m, 3H), 2.40 |
| | | | (d, J = 18.5 Hz, 3H), 1.41-1.33 (m, 3H); |
| | | | ¹⁹F NMR (376 MHz, CDCl₃) δ −62.11, −131.06, −131.45 |
| J462 | | ESIMS m/z 663 | ¹H NMR (400 MHz, CDCl₃) δ 8.04 (d, J = 8.5 Hz, 1H), 7.85 (d, |
| | | ([M + H]⁺) | J = 1.4 Hz, 1H), 7.68 (s, 1H), 7.62 (dd, J = 9.1, 4.6 Hz, 2H), |
| | | | 7.52-7.45 (m, 3H), 7.36 (d, J = 8.5 Hz, 2H), 7.09 (s, 1H), 6.79 |
| | | | (d, J = 9.0 Hz, 1H), 6.46 (d, J = 2.6 Hz, 1H), 4.13-3.78 (m, |
| | | | 2H), 3.07 (s, 6H), 2.25 (s, 3H); |
| | | | ¹⁹F NMR (376 MHz, CDCl₃) δ −58.04, −59.67 |
| J463 | | ESIMS m/z 610 | ¹H NMR (400 MHz, CDCl₃) δ 7.85 (dd, J = 4.6, 1.4 Hz, 2H), |
| | | ([M + H]⁺) | 7.79-7.74 (m, 2H), 7.73 (d, J = 1.4 Hz, 1H), 7.53 (dd, J = 7.6, |
| | | | 5.0 Hz, 2H), 7.54-7.48 (m, 1H), 7.49 (d, J = 2.3 Hz, 2H), 7.46 |
| | | | (d, J = 2.3 Hz, 1H), 7.40-7.33 (m, 2H), 6.94 (s, 1H), 4.17 (q, J = |
| | | | 6.2 Hz, 1H), 3.98 (dd, J = 3.9, 2.5 Hz, 2H), 3.26-3.02 (m, |
| | | | 3H), 2.42 (s, 3H), 1.37 (dd, J = 11.4, 6.5 Hz, 3H); |
| | | | ¹⁹F NMR (376 MHz, CDCl₃) δ −58.03 |
| J465 | | ESIMS m/z 693 | ¹H NMR (400 MHz, CDCl₃) δ 8.51 (s, 1H), 8.21 (dd, J = 8.9, 7.0 |
| | | ([M + H]⁺) | Hz, 3H), 7.56 (s, 1H), 7.53 (d, J = 7.9 Hz, 1H), 7.30 (d, J = 8.4 |
| | | | Hz, 2H), 7.29-7.27 (m, 1H), 7.11 (s, 1H), 7.04 (d, J = 1.9 Hz, |
| | | | 1H), 6.95 (d, J = 8.4 Hz, 1H), 4.11-4.01 (m, 2H), 3.94 (s, 2H), |
| | | | 2.38 (s, 3H), 2.30 (s, 3H), 2.25-2.11 (m, 2H), 2.05-1.94 (m, |
| | | | 2H); |
| | | | ¹⁹F NMR (376 MHz, CDCl₃) δ −57.70, −66.17 |
| J466 | | ESIMS m/z 609 | ¹H NMR (400 MHz, CDCl₃) δ 8.51 (s, 1H), 8.24-8.16 (m, 3H), |
| | | ([M + H]⁺) | 7.58-7.55 (m, 1H), 7.55-7.51 (m, 1H), 7.39 (d, J = 8.0 Hz, |
| | | | 1H), 7.34-7.28 (m, 3H), 7.13 (s, 1H), 6.92 (s, 1H), 3.98 (d, J = |
| | | | 1.2 Hz, 2H), 2.68 (p, J = 6.9 Hz, 1H), 2.39 (s, 3H), 2.28 (s, 3H), |
| | | | 1.21 (dd, J = 15.9, 6.8 Hz, 6H); |
| | | | ¹⁹F NMR (376 MHz, CDCl₃) δ −57.70 |
| J467 | | ESIMS m/z 625 | ¹H NMR (400 MHz, CDCl₃) δ 8.51 (d, J = 1.5 Hz, 1H), 8.24- |
| | | ([M + H]⁺) | 8.20 (m, 2H), 8.18 (dd, J = 8.6, 2.9 Hz, 1H), 7.58-7.51 (m, |
| | | | 3H), 7.38 (t, J = 6.6 Hz, 1H), 7.30 (d, J = 8.3 Hz, 2H), 7.06 (d, J = |
| | | | 15.9 Hz, 1H), 6.94 (s, 1H), 4.17 (dd, J = 15.1, 6.5 Hz, 1H), |
| | | | 4.02-3.94 (m, 2H), 3.17 (d, J = 38.5 Hz, 3H), 2.42 (s, 3H), |
| | | | 2.25 (d, J = 21.9 Hz, 3H), 1.38 (dd, J = 24.0, 6.4 Hz, 3H); |
| | | | ¹⁹F NMR (376 MHz, CDCl₃) δ −57.70 |
| J468 | | ESIMS m/z 664 | ¹H NMR (400 MHz, CDCl₃) δ 8.50 (s, 1H), 8.25-8.16 (m, 3H), |
| | | ([M + H]⁺) | 7.62 (d, J = 8.9 Hz, 1H), 7.55 (d, J = 2.5 Hz, 1H), 7.52 (dd, J = |
| | | | 8.7, 2.6 Hz, 1H), 7.30 (d, J = 8.3 Hz, 2H), 7.14 (s, 1H), 6.79 |
| | | | (dd, J = 9.0, 2.6 Hz, 1H), 6.46 (d, J = 2.6 Hz, 1H), 4.10-3.84 |
| | | | (m, 2H), 3.06 (s, 6H), 2.29 (s, 3H); |
| | | | ¹⁹F NMR (376 MHz, CDCl₃) δ −57.70, −59.63 |
| J469 | | ESIMS m/z 638 | ¹H NMR (400 MHz, CDCl₃) δ 8.51 (s, 1H), 8.26-8.17 (m, 3H), |
| | | ([M + H]⁺) | 7.59-7.51 (m, 2H), 7.37-7.28 (m, 3H), 7.20 (s, 1H), 6.89 |
| | | | (dd, J = 8.8, 2.7 Hz, 1H), 6.37 (d, J = 2.6 Hz, 1H), 3.98 (s, 2H), |
| | | | 2.97 (s, 6H), 2.67-2.55 (m, 1H), 2.28 (s, 3H), 1.18 (dd, J = |
| | | | 15.3, 6.8 Hz, 6H); |
| | | | ¹⁹F NMR (376 MHz, CDCl₃) δ −57.71 |
| J470 | | ESIMS m/z 656 | ¹H NMR (400 MHz, CDCl₃) δ 8.51 (s, 1H), 8.26-8.17 (m, 3H), |
| | | ([M + H]⁺) | 7.59-7.51 (m, 2H), 7.37-7.28 (m, 2H), 7.20 (s, 1H), 6.89 |
| | | | (dd, J = 8.8, 2.7 Hz, 1H), 6.37 (d, J = 2.6 Hz, 1H), 3.98 (s, 2H), |
| | | | 2.97 (s, 6H), 2.67-2.55 (m, 1H), 2.28 (s, 3H), 1.18 (dd, J = |
| | | | 15.3, 6.8 Hz, 6H); |
| | | | ¹⁹F NMR (376 MHz, CDCl₃) δ −57.70, −119.36 |
| J471 | | ESIMS m/z 625 | ¹H NMR (400 MHz, CDCl₃) δ 8.51 (s, 1H), 8.25-8.18 (m, 3H), |
| | | ([M + H]⁺) | 7.56 (s, 1H), 7.55-7.50 (m, 1H), 7.41 (d, J = 7.9 Hz, 1H), 7.30 |
| | | | (d, J = 8.2 Hz, 3H), 7.13 (s, 1H), 7.03 (s, 1H), 4.47-4.33 (m, |
| | | | 2H), 3.94 (s, 2H), 3.43 (q, J = 7.0 Hz, 2H), 2.43 (s, 3H), 2.29 (s, |
| | | | 3H), 1.16 (t, J = 7.0 Hz, 3H); |
| | | | ¹⁹F NMR (376 MHz, CDCl₃) δ −57.70 |
| J472 | | ESIMS m/z 639 | ¹H NMR (400 MHz, CDCl₃) δ 8.51 (s, 1H), 8.23-8.15 (m, 3H), |
| | | ([M + H]⁺) | 7.58-7.53 (m, 3H), 7.40-7.34 (m, 1H), 7.30 (d, J = 8.4 Hz, |
| | | | 2H), 7.07 (s, 1H), 6.93 (d, J = 1.9 Hz, 1H), 4.30 (q, J = 6.4 Hz, |
| | | | 1H), 3.98 (d, J = 1.2 Hz, 2H), 3.50-3.25 (m, 2H), 2.41 (s, 3H), |
| | | | 2.24 (s, 3H), 1.35 (d, J = 6.5 Hz, 3H), 1.15 (t, J = 7.0 Hz, 3H); |
| | | | ¹⁹F NMR (376 MHz, CDCl₃) δ −57.70 |
| J473 | | ESIMS m/z 612 | ¹H NMR (400 MHz, CDCl₃) δ 8.37 (t, J = 8.6 Hz, 1H), 7.84 (d, J = |
| | | ([M + H]⁺) | 1.3 Hz, 1H), 7.67 (ddd, J = 12.0, 8.3, 1.4 Hz, 2H), 7.58-7.51 |
| | | | (m, 2H), 7.52-7.42 (m, 3H), 7.37 (t, J = 7.5 Hz, 2H), 7.32 (d, J = |
| | | | 7.3 Hz, 1H), 6.90 (d, J = 1.7 Hz, 1H), 3.98 (d, J = 2.3 Hz, 2H), |
| | | | 2.66 (p, J = 6.8 Hz, 1H), 2.39 (s, 3H), 1.19 (dd, J = 9.1, 6.9 Hz, |
| | | | 6H); |
| | | | ¹⁹F NMR (376 MHz, CDCl₃) δ −58.04, −130.97 |
| J474 | | ESIMS m/z 641 | ¹H NMR (400 MHz, CDCl₃) δ 8.37 (t, J = 8.5 Hz, 1H), 7.84 (d, J = |
| | | ([M + H]⁺) | 1.4 Hz, 1H), 7.75-7.62 (m, 2H), 7.63-7.51 (m, 2H), 7.52- |
| | | | 7.42 (m, 3H), 7.37 (d, J = 8.5 Hz, 1H), 7.33 (d, J = 8.8 Hz, 1H), |
| | | | 6.89 (dd, J = 8.8, 2.8 Hz, 1H), 6.36 (d, J = 2.8 Hz, 1H), 3.98 (d, |
| | | | J = 1.7 Hz, 2H), 2.96 (s, 6H), 2.58 (p, J = 6.7 Hz, 1H), 1.17 (dd, |
| | | | J = 9.1, 6.9 Hz, 6H); |
| | | | ¹⁹F NMR (376 MHz, CDCl₃) δ −58.04, −130.67 |
| J476 | | ESIMS m/z 682 | ¹H NMR (400 MHz, CDCl₃) δ 8.38 (t, J = 8.4 Hz, 1H), 7.84 (d, J = |
| | | ([M + H]⁺) | 1.4 Hz, 1H), 7.67 (ddd, J = 12.1, 8.3, 1.4 Hz, 1H), 7.61-7.49 |
| | | | (m, 2H), 7.52-7.44 (m, 3H), 7.37 (d, J = 8.7 Hz, 2H), 7.28 (d, |
| | | | J = 7.5 Hz, 1H), 7.04-7.01 (m, 1H), 6.96 (d, J = 8.5 Hz, 1H), |
| | | | 4.21 (tt, J = 6.4, 3.0 Hz, 2H), 3.92 (d, J = 1.6 Hz, 2H), 2.62- |
| | | | 2.45 (m, 2H), 2.38 (s, 3H) |
| J477 | | ESIMS m/z 608 | ¹H NMR (400 MHz, CDCl₃) δ 8.03 (d, J = 8.4 Hz, 1H), 7.85 (d, |
| | | ([M + H]⁺) | J = 1.4 Hz, 1H), 7.68 (t, J = 1.7 Hz, 1H), 7.65-7.59 (m, 1H), |
| | | | 7.51-7.43 (m, 3H), 7.40-7.29 (m, 4H), 7.12 (s, 1H), 6.92 (d, |
| | | | J = 1.7 Hz, 1H), 3.96 (d, J = 1.0 Hz, 2H), 2.68 (dq, J = 13.1, 6.5 |
| | | | Hz, 1H), 2.39 (s, 3H), 2.23 (s, 3H), 1.24-1.16 (m, 6H); |
| | | | ¹⁹F NMR (376 MHz, CDCl₃) δ −58.04 |
| J480 | | ESIMS m/z 678 | ¹H NMR (400 MHz, CDCl₃) δ 8.02 (d, J = 8.4 Hz, 1H), 7.85 (d, |
| | | ([M + H]⁺) | J = 1.3 Hz, 1H), 7.67 (d, J = 7.7 Hz, 2H), 7.62 (d, J = 8.6 Hz, |
| | | | 1H), 7.54-7.44 (m, 3H), 7.41-7.33 (m, 3H), 7.04 (s, 1H), |
| | | | 6.96 (s, 1H), 4.51 (d, J = 6.3 Hz, 1H), 4.05-3.86 (m, 2H), 3.45 |
| | | | (s, 3H), 2.46 (s, 3H), 2.17 (d, J = 3.0 Hz, 3H); |
| | | | ¹⁹F NMR (376 MHz, CDCl₃) δ −58.03, −76.43 |
| J481 | | ESIMS m/z 626 | ¹H NMR (400 MHz, CDCl₃) δ 8.77 (s, 1H), 8.43 (d, J = 1.3 Hz, |
| | | ([M + H]⁺) | 1H), 8.40 (t, J = 8.5 Hz, 1H), 8.08 (dd, J = 8.6, 2.4 Hz, 1H), |
| | | | 7.93 (d, J = 1.3 Hz, 1H), 7.62-7.55 (m, 3H), 7.51 (d, J = 8.6 |
| | | | Hz, 1H), 7.33 (d, J = 8.8 Hz, 1H), 6.89 (dd, J = 8.8, 2.7 Hz, 1H), |
| | | | 6.36 (d, J = 2.7 Hz, 1H), 3.98 (d, J = 1.7 Hz, 2H), 2.96 (s, 6H), |
| | | | 2.58 (p, J = 6.8 Hz, 1H), 1.17 (dd, J = 9.1, 6.8 Hz, 6H); |
| | | | ¹⁹F NMR (376 MHz, CDCl₃) δ −62.08, −130.67 |
| J482 | | ESIMS m/z 597 | ¹H NMR (400 MHz, CDCl₃) δ 8.77 (s, 1H), 8.43 (d, J = 1.3 Hz, |
| | | ([M + H]⁺) | 1H), 8.39 (t, J = 8.6 Hz, 1H), 8.09 (dd, J = 8.7, 2.4 Hz, 1H), |
| | | | 7.93 (d, J = 1.3 Hz, 1H), 7.63-7.57 (m, 2H), 7.51 (d, J = 8.4 |
| | | | Hz, 2H), 7.38 (d, J = 8.0 Hz, 1H), 7.32 (d, J = 7.9 Hz, 1H), 6.90 |
| | | | (s, 1H), 3.98 (d, J = 2.3 Hz, 2H), 2.66 (p, J = 7.0 Hz, 1H), 2.39 |
| | | | (s, 3H), 1.19 (dd, J = 9.2, 6.8 Hz, 6H); |
| | | | ¹⁹F NMR (376 MHz, CDCl₃) δ −62.09, −130.97 |
| J483 | | ESIMS m/z 652 | ¹H NMR (400 MHz, CDCl₃) δ 8.77 (d, J = 2.1 Hz, 1H), 8.44 (d, |
| | | ([M + H]⁺) | J = 1.3 Hz, 1H), 8.41 (t, J = 8.3 Hz, 1H), 8.08 (dd, J = 8.7, 2.4 |
| | | | Hz, 1H), 7.93 (d, J = 1.4 Hz, 1H), 7.65-7.56 (m, 3H), 7.51 (d, |
| | | | J = 8.9 Hz, 2H), 6.80 (dd, J = 9.0, 2.6 Hz, 1H), 6.44 (d, J = 2.6 |
| | | | Hz, 1H), 3.96 (q, J = 18.0 Hz, 2H), 3.07 (s, 6H); |
| | | | ¹⁹F NMR (376 MHz, CDCl₃) δ −59.67, −62.09, −131.34 |
| J484 | | ESIMS m/z 667 | ¹H NMR (400 MHz, CDCl₃) δ 8.77 (d, J = 2.1 Hz, 1H), 8.46- |
| | | ([M + H]⁺) | 8.34 (m, 2H), 8.09 (dd, J = 8.6, 2.4 Hz, 1H), 7.93 (d, J = 1.3 Hz, |
| | | | 1H), 7.66-7.54 (m, 2H), 7.51 (d, J = 8.5 Hz, 1H), 7.47 (d, J = |
| | | | 2.9 Hz, 1H), 7.28 (dd, J = 8.7, 2.0 Hz, 1H), 7.06-7.01 (m, 1H), |
| | | | 6.97 (d, J = 8.4 Hz, 1H), 4.22 (tt, J = 6.6, 3.1 Hz, 2H), 3.93 (d, J = |
| | | | 1.6 Hz, 2H), 2.59-2.47 (m, 2H), 2.38 (s, 3H); |
| | | | ¹⁹F NMR (376 MHz, CDCl₃) δ −62.08, −64.59, −131.43 |
| J485 | | ESIMS m/z 667 | ¹H NMR (400 MHz, CDCl₃) δ 8.77 (d, J = 2.4 Hz, 1H), 8.43 (d, |
| | | ([[M + H]⁺) | J = 1.4 Hz, 1H), 8.35 (t, J = 8.4 Hz, 1H), 8.09 (dd, J = 8.6, 2.4 |
| | | | Hz, 1H), 7.94 (d, J = 1.3 Hz, 1H), 7.68 (d, J = 8.0 Hz, 1H), 7.64- |
| | | | 7.56 (m, 2H), 7.51 (d, J = 8.5 Hz, 1H), 7.51-7.40 (m, 1H), |
| | | | 7.33 (d, J = 3.0 Hz, 1H), 7.02 (s, 1H), 4.48 (q, J = 6.3 Hz, 1H), |
| | | | 3.99 (d, J = 8.6 Hz, 2H), 3.42 (s, 3H), 2.46 (s, 3H); |
| | | | ¹⁹F NMR (376 MHz, CDCl₃) δ −62.09, −76.44, −131.84 |
| J486 | | ESIMS m/z 638 | ¹H NMR (400 MHz, CDCl₃) δ 8.02 (d, J = 8.4 Hz, 1H), 7.85 (d, |
| | | ([M + H]⁺) | J = 1.3 Hz, 1H), 7.68 (s, 1H), 7.67-7.59 (m, 1H), 7.56 (d, J = |
| | | | 8.0 Hz, 1H), 7.51-7.44 (m, 3H), 7.36 (d, J = 8.6 Hz, 3H), 7.02 |
| | | | (s, 1H), 6.93 (s, 1H), 4.33-4.24 (m, 1H), 3.96 (s, 2H), 3.46- |
| | | | 3.24 (m, 2H), 2.41 (s, 3H), 2.19 (s, 3H), 1.34 (d, J = 6.5 Hz, |
| | | | 3H), 1.15 (t, J = 7.0 Hz, 3H) |
| J487 | | ESIMS m/z 667 | ¹H NMR (400 MHz, CDCl₃) δ 8.76 (dt, J = 1.9, 0.9 Hz, 1H), |
| | | ([M + H]⁺) | 8.42 (d, J = 1.3 Hz, 1H), 8.38 (t, J = 8.5 Hz, 1H), 8.08 (dd, J = |
| | | | 8.8, 2.4 Hz, 1H), 7.92 (d, J = 1.3 Hz, 1H), 7.63-7.55 (m, 2H), |
| | | | 7.50 (d, J = 8.5 Hz, 1H), 7.46-7.43 (m, 1H), 7.41 (d, J = 7.9 |
| | | | Hz, 1H), 7.34 (dd, J = 8.0, 1.6 Hz, 1H), 7.06-7.02 (m, 1H), |
| | | | 4.62 (d, J = 12.4 Hz, 1H), 4.49 (d, J = 12.4 Hz, 1H), 3.95 (d, J = |
| | | | 2.6 Hz, 2H), 3.71 (qd, J = 8.7, 4.1 Hz, 2H), 2.44 (s, 3H); |
| | | | ¹⁹F NMR (376 MHz, CDCl₃) δ −62.08, −73.78, −131.30 |
| J488 | | HRMS-ESI (m/z) | ¹H NMR (400 MHz, CDCl₃) δ 8.53 (s, 1H), 8.19 (d, J = 8.5 Hz, |
| | | [M + H]⁺ calcd for | 1H), 8.02 (d, J = 8.6 Hz, 1H), 7.96 (d, J = 2.0 Hz, 1H), 7.82- |
| | | C₃₁H₂₃F₃N₆O₄S, | 7.76 (m, 2H), 7.73 (d, J = 8.1 Hz, 1H), 7.45-7.41 (m, 1H), |
| | | 633.1526; found, | 7.41-7.33 (m, 3H), 7.07 (s, 2H), 6.48 (d, J = 3.4 Hz, 1H), 6.44 |
| | | 633.1514 | (dd, J = 3.4, 1.8 Hz, 1H), 3.94 (s, 2H), 2.45 (s, 3H), 2.24 (s, |
| | | | 3H); |
| | | | ¹⁹F NMR (376 MHz, CDCl₃) δ −58.03 |
| J489 | | HRMS-ESI (m/z) | ¹H NMR (400 MHz, CDCl₃) δ 8.54 (s, 1H), 8.47 (t, J = 8.4 Hz, |
| | | [M + H]⁺ calcd for | 1H), 7.95 (d, J = 8.6 Hz, 1H), 7.87 (dd, J = 12.1, 1.9 Hz, 1H), |
| | | C₃₀H₂₀F₄N₆O₄S, | 7.80-7.76 (m, 2H), 7.74 (d, J = 8.1 Hz, 1H), 7.50 (d, J = 3.0 |
| | | 637.1276; found, | Hz, 1H), 7.42 (dd, J = 1.9, 0.7 Hz, 1H), 7.37 (t, J = 8.2 Hz, 3H), |
| | | 637.1267 | 7.05 (s, 1H), 6.49 (d, J = 3.4 Hz, 1H), 6.43 (dd, J = 3.4, 1.8 Hz, |
| | | | 1H), 3.96 (s, 2H), 2.46 (s, 3H); |
| | | | ¹⁹F NMR (376 MHz, CDCl₃) δ −58.02, −131.20 |
| J490 | | ESIMS m/z 665 | ¹H NMR (400 MHz, CDCl₃) δ 8.13 (s, 1H), 7.87-7.81 (m, 4H), |
| | | ([M + H]⁺) | 7.60 (d, J = 8.6 Hz, 2H), 7.42 (t, J = 8.5 Hz, 3H), 7.35 (d, J = |
| | | | 7.9 Hz, 1H), 7.30 (s, 1H), 7.05 (s, 1H), 4.62 (d, J = 12.3 Hz, |
| | | | 1H), 4.50 (d, J = 12.5 Hz, 1H), 3.95 (d, J = 2.2 Hz, 2H), 3.78- |
| | | | 3.68 (m, 2H), 2.45 (s, 3H); |
| | | | ¹⁹F NMR (376 MHz, CDCl₃) δ −57.97, −73.76 |
| J491 | | ESIMS m/z 665 | ¹H NMR (400 MHz, CDCl₃) δ 8.13 (s, 1H), 7.84 (d, J = 8.9 Hz, |
| | | ([M + H]⁺) | 4H), 7.61 (d, J = 8.7 Hz, 2H), 7.45-7.38 (m, 2H), 7.33 (s, 1H), |
| | | | 7.29 (d, J = 8.9 Hz, 1H), 7.04 (d, J = 2.2 Hz, 1H), 6.97 (d, J = |
| | | | 8.5 Hz, 1H), 4.22 (td, J = 6.3, 2.1 Hz, 2H), 3.93 (d, J = 1.7 Hz, |
| | | | 2H), 2.63-2.46 (m, 2H), 2.38 (s, 3H); |
| | | | ¹⁹F NMR (376 MHz, CDCl₃) δ −57.97, −64.58 |
| J492 | | ESIMS m/z 679 | ¹H NMR (400 MHz, CDCl₃) δ 8.13 (s, 1H), 7.84 (d, J = 8.8 Hz, |
| | | ([M + H]⁺) | 3H), 7.60 (d, J = 8.6 Hz, 2H), 7.41 (d, J = 8.6 Hz, 2H), 7.35 (s, |
| | | | 1H), 7.30-7.28 (m, 1H), 7.04 (d, J = 2.2 Hz, 1H), 6.95 (d, J = |
| | | | 8.4 Hz, 1H), 4.05 (q, J = 5.6 Hz, 2H), 3.94 (s, 2H), 2.38 (s, 3H), |
| | | | 2.18 (td, J = 10.6, 6.4 Hz, 2H), 2.08-1.93 (m, 2H); |
| | | | ¹⁹F NMR (376 MHz, CDCl₃) δ −57.97, −66.22 |
| J493 | | ESIMS m/z 611 | ¹H NMR (400 MHz, CDCl₃) δ 8.13 (s, 1H), 7.84 (d, J = 9.0 Hz, |
| | | ([M + H]⁺) | 4H), 7.60 (d, J = 8.7 Hz, 2H), 7.47-7.37 (m, 4H), 7.31 (d, J = |
| | | | 8.0 Hz, 1H), 7.02 (s, 1H), 4.52-4.29 (m, 2H), 3.95 (s, 2H), |
| | | | 3.51-3.38 (m, 2H), 2.43 (s, 3H), 1.16 (t, J = 7.0 Hz, 3H); |
| | | | ¹⁹F NMR (376 MHz, CDCl₃) δ −57.97 |
| J494 | | ESIMS m/z 665 | ¹H NMR (500 MHz, CDCl₃) δ 8.14 (s, 1H), 7.87-7.81 (m, 4H), |
| | | ([M + H]⁺) | 7.70-7.65 (m, 1H), 7.57 (d, J = 8.7 Hz, 2H), 7.42 (t, J = 9.6 |
| | | | Hz, 3H), 7.23 (s, 1H), 7.03 (s, 1H), 4.46 (q, J = 6.2 Hz, 1H), |
| | | | 4.08-3.90 (m, 2H), 3.43 (s, 3H), 2.46 (s, 3H); |
| | | | ¹⁹F NMR (471 MHz, CDCl₃) δ −57.96, −76.21 |
| J495 | | ESIMS m/z 650 | ¹H NMR (500 MHz, CDCl₃) δ 8.13 (s, 1H), 7.84 (d, J = 8.9 Hz, |
| | | ([M + H]⁺) | 3H), 7.62 (t, J = 9.2 Hz, 4H), 7.41 (d, J = 8.5 Hz, 2H), 7.35 (s, |
| | | | 1H), 6.80 (dd, J = 9.2, 2.7 Hz, 1H), 6.45 (d, J = 2.6 Hz, 1H), |
| | | | 4.06-3.86 (m, 2H), 3.07 (s, 6H); |
| | | | ¹⁹F NMR (471 MHz, CDCl₃) δ −57.96, −59.66 |
| J496 | | ESIMS m/z 624 | ¹H NMR (500 MHz, CDCl₃) δ 8.13 (s, 1H), 7.84 (dd, J = 8.8, 1.1 |
| | | ([M − H]⁻) | Hz, 4H), 7.61 (d, J = 8.6 Hz, 2H), 7.46 (s, 1H), 7.41 (d, J = 8.5 |
| | | | Hz, 2H), 7.34 (d, J = 8.6 Hz, 1H), 6.80 (dd, J = 8.6, 2.7 Hz, 1H), |
| | | | 6.48 (d, J = 2.7 Hz, 1H), 4.33 (d, J = 12.2 Hz, 1H), 4.20 (d, J = |
| | | | 12.1 Hz, 1H), 3.95 (d, J = 8.6 Hz, 2H), 3.24 (s, 3H), 3.00 (s, |
| | | | 6H); |
| | | | ¹⁹F NMR (471 MHz, CDCl₃) δ −57.96 |
| J497 | | HRMS-ESI (m/z) | ¹H NMR (400 MHz, CDCl₃) δ 8.63 (s, 1H), 8.50 (t, J = 8.4 Hz, |
| | | [M + H]⁺ calcd for | 1H), 8.02-7.94 (m, 1H), 7.92 (m, 2H), 7.87 (d, J = 8.7 Hz, |
| | | C₂₉H₂₃F₉N₆O₃S2, | 3H), 7.50 (d, J = 3.0 Hz, 1H), 7.28 (s, 1H), 6.99 (s, 1H), 4.60 (d, |
| | | 739.120; found, | J = 12.4 Hz, 1H), 4.47 (d, J = 12.4 Hz, 1H), 3.95 (d, J = 2.4 Hz, |
| | | 739.1197 | 2H), 3.71 (qd, J = 8.7, 3.1 Hz, 2H), 2.35 (d, J = 10.1 Hz, 6H) |
| J498 | | HRMS-ESI (m/z) | ¹H NMR (400 MHz, CDCl₃) δ 8.63 (s, 1H), 8.50 (t, J = 8.4 Hz, |
| | | [M + H]⁺ calcd for | 1H), 8.03-7.96 (m, 1H), 7.96-7.90 (m, 3H), 7.87 (d, J = 9.0 |
| | | C₂₈H₂₄F₆N₆O₂S2, | Hz, 3H), 7.53 (d, J = 3.0 Hz, 1H), 7.37-7.26 (m, 3H), 6.93 (s, |
| | | 655.1379; found, | 1H), 3.99 (d, J = 2.5 Hz, 2H), 2.39 (d, J = 7.6 Hz, 3H), 1.60 (dt, |
| | | 655.1366 | J = 15.3, 7.5 Hz, 2H), 0.93 (t, J = 7.3 Hz, 3H) |
| J499 | | ESIMS m/z 630 | ¹H NMR (500 MHz, CDCl₃) δ 8.54 (t, J = 8.2 Hz, 1H), 8.07- |
| | | ([M + H]⁺) | 8.01 (m, 2H), 7.83 (dd, J = 11.5, 2.1 Hz, 1H), 7.69 (d, J = 8.6 |
| | | | Hz, 1H), 7.62 (s, 1H), 7.45-7.31 (m, 4H), 6.94-6.86 (m, 1H), |
| | | | 4.00 (d, J = 3.8 Hz, 2H), 2.65 (p, J = 6.8 Hz, 1H), 2.39 (s, 3H), |
| | | | 1.19 (dd, J = 9.9, 6.8 Hz, 6H); |
| | | | ¹⁹F NMR (471 MHz, CDCl₃) δ −57.68, −129.72 |
| J500 | | ESIMS m/z 700 | ¹H NMR (500 MHz, CDCl₃) δ 8.54 (t, J = 8.3 Hz, 1H), 8.08- |
| | | ([M + H]⁺) | 8.02 (m, 2H), 7.84 (dd, J = 11.7, 2.0 Hz, 1H), 7.69 (dd, J = 8.6, |
| | | | 1.9 Hz, 1H), 7.56 (d, J = 3.2 Hz, 1H), 7.42 (d, J = 7.8 Hz, 1H), |
| | | | 7.35 (d, J = 8.4 Hz, 3H), 7.08-7.03 (m, 1H), 4.63 (d, J = 12.4 |
| | | | Hz, 1H), 4.50 (d, J = 12.4 Hz, 1H), 3.97 (d, J = 4.2 Hz, 2H), |
| | | | 3.72 (qt, J = 8.6, 4.3 Hz, 2H), 2.46 (s, 3H); |
| | | | ¹⁹F NMR (471 MHz, CDCl₃) δ −57.68, −73.78, −130.11 |
| J501 | | ESIMS m/z 700 | ¹H NMR (500 MHz, CDCl₃) δ 8.55 (t, J = 8.3 Hz, 1H), 8.08- |
| | | ([M + H]⁺) | 8.00 (m, 2H), 7.84 (dd, J = 11.6, 2.0 Hz, 1H), 7.72-7.66 (m, |
| | | | 1H), 7.59 (d, J = 3.2 Hz, 1H), 7.35 (d, J = 8.3 Hz, 2H), 7.30 (d, |
| | | | J = 8.5 Hz, 1H), 7.05-7.00 (m, 1H), 6.97 (d, J = 8.5 Hz, 1H), |
| | | | 4.22 (tt, J = 10.0, 5.1 Hz, 2H), 3.95 (d, J = 2.3 Hz, 2H), 2.60- |
| | | | 2.48 (m, 2H), 2.38 (s, 3H); |
| | | | ¹⁹F NMR (471 MHz, CDCl₃) δ −57.68, −64.57, −130.22 |
| J502 | | ESIMS m/z 712 | ¹H NMR (500 MHz, CDCl₃) δ 8.54 (t, J = 8.3 Hz, 1H), 8.07- |
| | | ([M − H]⁻) | 8.01 (m, 2H), 7.84 (dd, J = 11.7, 1.9 Hz, 1H), 7.70-7.67 (m, |
| | | | 1H), 7.60 (d, J = 3.1 Hz, 1H), 7.35 (d, J = 8.3 Hz, 2H), 7.31- |
| | | | 7.27 (m, 1H), 7.04-7.00 (m, 1H), 6.95 (d, J = 8.5 Hz, 1H), |
| | | | 4.05 (q, J = 5.4 Hz, 2H), 3.96 (d, J = 1.1 Hz, 2H), 2.38 (s, 3H), |
| | | | 2.25-2.10 (m, 2H), 2.03-1.93 (m, 2H); |
| | | | ¹⁹F NMR (471 MHz, CDCl₃) δ −57.68, −66.30, −130.14 |
| J503 | | ESIMS m/z 646 | ¹H NMR (500 MHz, CDCl₃) δ 8.54 (t, J = 8.3 Hz, 1H), 8.08- |
| | | ([M + H]⁺) | 8.01 (m, 2H), 7.83 (dd, J = 11.6, 2.0 Hz, 1H), 7.73-7.65 (m, |
| | | | 1H), 7.60 (s, 1H), 7.40 (d, J = 7.9 Hz, 1H), 7.33 (dd, J = 18.7, |
| | | | 8.2 Hz, 3H), 7.01 (s, 1H), 4.43 (d, J = 12.6 Hz, 1H), 4.36 (d, J = |
| | | | 12.5 Hz, 1H), 3.96 (d, J = 1.3 Hz, 2H), 3.41 (q, J = 7.0 Hz, 2H), |
| | | | 2.43 (s, 3H), 1.14 (t, J = 7.0 Hz, 3H); |
| | | | ¹⁹F NMR (471 MHz, CDCl₃) δ −57.68, −129.89 |
| J504 | | ESIMS m/z 646 | ¹H NMR (500 MHz, CDCl₃) δ 8.57-8.45 (m, 1H), 8.09-8.01 |
| | | ([M + H]⁺) | (m, 2H), 7.82 (dd, J = 11.6, 2.4 Hz, 1H), 7.69 (d, J = 8.6 Hz, |
| | | | 1H), 7.57-7.50 (m, 2H), 7.37 (dd, J = 17.9, 8.2 Hz, 3H), 6.93 |
| | | | (s, 1H), 4.21-4.08 (m, 1H), 4.01 (dd, J = 4.8, 2.3 Hz, 2H), |
| | | | 3.15 (d, J = 26.2 Hz, 3H), 2.43 (s, 3H), 1.37 (dd, J = 17.9, 6.4 |
| | | | Hz, 3H); |
| | | | ¹⁹F NMR (471 MHz, CDCl₃) δ −57.68, −129.82, −130.23 |
| J505 | | ESIMS m/z 700 | ¹H NMR (500 MHz, CDCl₃) δ 8.50 (t, J = 8.3 Hz, 1H), 8.07- |
| | | ([M + H]⁺) | 8.01 (m, 2H), 7.83 (dd, J = 11.6, 2.0 Hz, 1H), 7.69 (t, J = 8.7 |
| | | | Hz, 2H), 7.44 (d, J = 7.1 Hz, 2H), 7.35 (d, J = 8.3 Hz, 2H), 7.07- |
| | | | 6.98 (m, 1H), 4.46 (q, J = 6.3 Hz, 1H), 4.11-3.92 (m, 2H), |
| | | | 3.42 (s, 3H), 2.47 (s, 3H); |
| | | | ¹⁹F NMR (471 MHz, CDCl₃) δ −57.68, −76.37, −130.63 |
| J506 | | ESIMS m/z 659 | ¹H NMR (500 MHz, CDCl₃) δ 8.55 (t, J = 8.3 Hz, 1H), 8.07- |
| | | ([M + H]⁺) | 8.02 (m, 2H), 7.82 (dd, J = 11.4, 2.0 Hz, 1H), 7.69 (d, J = 6.5 |
| | | | Hz, 2H), 7.34 (dd, J = 8.7, 6.4 Hz, 3H), 6.90 (dd, J = 8.8, 2.7 |
| | | | Hz, 1H), 6.35 (d, J = 2.8 Hz, 1H), 4.00 (d, J = 2.8 Hz, 2H), 2.97 |
| | | | (s, 6H), 2.57 (p, J = 6.8 Hz, 1H), 1.17 (dd, J = 10.2, 6.8 Hz, |
| | | | 6H); |
| | | | ¹⁹F NMR (471 MHz, CDCl₃) δ −57.68, −129.42 |
| J507 | | ESIMS m/z 677 | ¹H NMR (500 MHz, CDCl₃) δ 8.53 (t, J = 8.3 Hz, 1H), 8.07- |
| | | ([M + H]⁺) | 8.03 (m, 2H), 7.83 (dd, J = 11.5, 2.0 Hz, 1H), 7.70 (d, J = 8.7 |
| | | | Hz, 1H), 7.63 (d, J = 3.0 Hz, 1H), 7.35 (d, J = 8.4 Hz, 2H), 7.09 |
| | | | (d, J = 14.0 Hz, 1H), 6.52 (d, J = 8.3 Hz, 1H), 4.00 (d, J = 2.7 |
| | | | Hz, 2H), 2.87 (s, 6H), 2.64-2.53 (m, 1H), 1.16 (t, J = 6.9 Hz, |
| | | | 6H); |
| | | | ¹⁹F NMR (471 MHz, CDCl₃) δ −57.68, −119.15, −129.75 |
| J508 | | | ¹H NMR (400 MHz, CDCl₃) δ 8.76 (s, 1H), 8.43 (d, J = 1.1 Hz, |
| | | | 1H), 8.15-7.99 (m, 2H), 7.91 (s, 1H), 7.70 (s, 1H), 7.66 (d, J = |
| | | | 9.0 Hz, 1H), 7.60-7.31 (m, 3H), 7.04 (d, J = 18.3 Hz, 1H), |
| | | | 6.94 (s, 1H), 4.25-4.07 (m, 1H), 3.97 (d, J = 2.5 Hz, 2H), 3.16 |
| | | | (d, J = 38.6 Hz, 3H), 2.42 (d, J = 1.9 Hz, 3H), 2.20 (d, J = 22.6 |
| | | | Hz, 3H), 1.47-1.31 (m, 3H); |
| | | | ¹⁹F NMR (376 MHz, CDCl₃) δ −62.07 |
| J509 | | ESIMS m/z 593 | ¹H NMR (400 MHz, CDCl₃) δ 8.76 (s, 1H), 8.44 (d, J = 1.3 Hz, |
| | | ([M + H]⁺) | 1H), 8.12-8.02 (m, 2H), 7.91 (d, J = 1.3 Hz, 1H), 7.71 (s, 1H), |
| | | | 7.68-7.59 (m, 1H), 7.50 (d, J = 8.7 Hz, 1H), 7.38 (d, J = 8.1 |
| | | | Hz, 1H), 7.35-7.28 (m, 1H), 7.12 (s, 1H), 6.94-6.88 (m, 1H), |
| | | | 3.97 (s, 2H), 2.68 (p, J = 6.9 Hz, 1H), 2.39 (s, 3H), 2.23 (s, 3H), |
| | | | 1.20 (dd, J = 16.7, 6.8 Hz, 6H); |
| | | | ¹⁹F NMR (376 MHz, CDCl₃) δ −62.07 |
| J510 | | ESIMS m/z 622 | ¹H NMR (400 MHz, CDCl₃) δ 8.76 (s, 1H), 8.44 (d, J = 1.3 Hz, |
| | | ([M + H]⁺) | 1H), 8.12-8.02 (m, 2H), 7.91 (d, J = 1.3 Hz, 1H), 7.71 (s, 1H), |
| | | | 7.70-7.60 (m, 1H), 7.50 (d, J = 8.6 Hz, 1H), 7.33 (d, J = 8.8 |
| | | | Hz, 1H), 7.19 (s, 1H), 6.88 (dd, J = 8.8, 2.8 Hz, 1H), 6.38 (d, J = |
| | | | 2.7 Hz, 1H), 3.96 (s, 2H), 2.96 (s, 6H), 2.61 (p, J = 6.8 Hz, |
| | | | 1H), 2.24 (s, 3H), 1.18 (dd, J = 15.3, 6.8 Hz, 6H); |
| | | | ¹⁹F NMR (376 MHz, CDCl₃) δ −62.06 |
| J511 | | ESIMS m/z 663 | ¹H NMR (400 MHz, CDCl₃) δ 8.76 (s, 1H), 8.44 (d, J = 1.3 Hz, |
| | | ([M + H]⁺) | 1H), 8.06 (t, J = 8.8 Hz, 2H), 7.91 (d, J = 1.4 Hz, 1H), 7.71 (s, |
| | | | 1H), 7.69-7.61 (m, 1H), 7.51 (d, J = 8.7 Hz, 1H), 7.43 (d, J = |
| | | | 7.9 Hz, 1H), 7.34 (d, J = 7.8 Hz, 1H), 7.05 (s, 2H), 4.63 (d, J = |
| | | | 12.5 Hz, 1H), 4.51 (d, J = 12.4 Hz, 1H), 3.93 (d, J = 2.1 Hz, |
| | | | 2H), 3.82-3.66 (m, 2H), 2.44 (s, 3H), 2.23 (s, 3H); |
| | | | ¹⁹F NMR (376 MHz, CDCl₃) δ −62.07, −73.75 |
| J512 | | ESIMS m/z 663 | ¹H NMR (400 MHz, CDCl₃) δ 8.76 (s, 1H), 8.44 (d, J = 1.3 Hz, |
| | | ([M + H]⁺) | 1H), 8.14-8.03 (m, 2H), 7.91 (d, J = 1.4 Hz, 1H), 7.71 (s, 1H), |
| | | | 7.65 (d, J = 8.7 Hz, 1H), 7.51 (d, J = 8.6 Hz, 1H), 7.31-7.26 |
| | | | (m, 1H), 7.11-7.02 (m, 2H), 6.96 (d, J = 8.5 Hz, 1H), 4.22 (t, J = |
| | | | 6.3 Hz, 2H), 3.91 (d, J = 1.8 Hz, 2H), 2.55 (ddt, J = 15.9, |
| | | | 10.4, 4.7 Hz, 2H), 2.38 (s, 3H), 2.25 (s, 3H); |
| | | | ¹⁹F NMR (376 MHz, CDCl₃) δ −62.06, −64.56 |
| J513 | | ESIMS m/z 594 | ¹H NMR (400 MHz, CDCl₃) δ 7.94 (d, J = 1.4 Hz, 1H), 7.77 (dd, |
| | | ([M + H]⁺) | J = 8.5, 1.9 Hz, 4H), 7.59-7.51 (m, 6H), 7.38 (t, J = 6.9 Hz, |
| | | | 1H), 7.29 (d, J = 14.3 Hz, 1H), 6.94 (s, 1H), 4.16 (p, J = 6.2 Hz, |
| | | | 1H), 3.98 (dd, J = 3.8, 2.6 Hz, 2H), 3.15 (d, J = 20.4 Hz, 3H), |
| | | | 2.42 (s, 3H), 1.37 (dd, J = 11.4, 6.4 Hz, 3H); |
| | | | ¹⁹F NMR (376 MHz, CDCl₃) δ −62.46 |
| J514 | | ESIMS m/z 578 | ¹H NMR (400 MHz, CDCl₃) δ 7.94 (d, J = 1.4 Hz, 1H), 7.77 (dd, |
| | | ([M + H]⁺) | J = 8.6, 2.1 Hz, 4H), 7.58-7.53 (m, 5H), 7.38 (t, J = 7.9 Hz, |
| | | | 2H), 7.32 (d, J = 8.0 Hz, 1H), 6.92 (d, J = 1.7 Hz, 1H), 3.97 (d, |
| | | | J = 3.0 Hz, 2H), 2.68 (p, J = 6.8 Hz, 1H), 2.39 (s, 3H), 1.19 (dd, |
| | | | J = 6.8 Hz, 6H); |
| | | | ¹⁹F NMR (376 MHz, CDCl₃) δ −62.46 |
| J515 | | ESIMS m/z 607 | ¹H NMR (400 MHz, CDCl₃) δ 7.94 (d, J = 1.4 Hz, 1H), 7.77 (dd, |
| | | ([M + H]⁺) | J = 8.7, 2.2 Hz, 4H), 7.61-7.51 (m, 5H), 7.43 (s, 1H), 7.33 (d, |
| | | | J = 8.8 Hz, 1H), 6.89 (dd, J = 8.8, 2.8 Hz, 1H), 6.37 (d, J = 2.8 |
| | | | Hz, 1H), 3.97 (d, J = 2.4 Hz, 2H), 2.96 (s, 6H), 2.60 (p, J = 6.9 |
| | | | Hz, 1H), 1.17 (dd, J = 6.8, 5.5 Hz, 6H); |
| | | | ¹⁹F NMR (376 MHz, CDCl₃) δ −62.46 |
| J516 | | ESIMS m/z 648 | ¹H NMR (400 MHz, CDCl₃) δ 7.93 (d, J = 1.4 Hz, 1H), 7.77 (dd, |
| | | ([M + H]⁺) | J = 8.8, 2.0 Hz, 4H), 7.59-7.52 (m, 5H), 7.42 (d, J = 7.8 Hz, |
| | | | 1H), 7.34 (d, J = 7.4 Hz, 1H), 7.28 (s, 1H), 7.05 (d, J = 1.6 Hz, |
| | | | 1H), 4.62 (d, J = 12.3 Hz, 1H), 4.50 (d, J = 12.3 Hz, 1H), 3.94 |
| | | | (d, J = 2.2 Hz, 2H), 3.73 (qd, J = 8.7, 1.9 Hz, 2H), 2.44 (s, 3H); |
| | | | ¹⁹F NMR (376 MHz, CDCl₃) δ −62.46, −73.76 |
| J517 | | ESIMS m/z 648 | ¹H NMR (400 MHz, CDCl₃) δ 7.94 (d, J = 1.4 Hz, 1H), 7.77 (dd, |
| | | ([M + H]⁺) | J = 8.8, 2.4 Hz, 4H), 7.64-7.51 (m, 5H), 7.33-7.26 (m, 2H), |
| | | | 7.04 (d, J = 2.2 Hz, 1H), 6.96 (d, J = 8.5 Hz, 1H), 4.21 (td, J = |
| | | | 6.3, 1.7 Hz, 2H), 3.92 (d, J = 1.8 Hz, 2H), 2.61-2.48 (m, 2H), |
| | | | 2.37 (s, 3H); |
| | | | ¹⁹F NMR (376 MHz, CDCl₃) δ −62.45, −64.58 |
| J518 | | ESIMS m/z 595 | ¹H NMR (400 MHz, CDCl₃) δ 8.76 (d, J = 2.3 Hz, 1H), 8.43 (d, |
| | | ([M + H]⁺) | J = 1.4 Hz, 1H), 8.07 (dd, J = 8.8, 2.4 Hz, 1H), 7.91 (d, J = 1.4 |
| | | | Hz, 1H), 7.79 (dd, J = 8.7, 1.4 Hz, 2H), 7.59-7.47 (m, 4H), |
| | | | 7.38 (t, J = 6.8 Hz, 1H), 7.31 (d, J = 12.6 Hz, 1H), 6.94 (s, 1H), |
| | | | 4.16 (p, J = 6.2 Hz, 1H), 3.98 (dd, J = 3.8, 2.6 Hz, 2H), 3.15 (d, |
| | | | J = 20.3 Hz, 3H), 2.42 (s, 3H), 1.37 (dd, J = 11.4, 6.4 Hz, 3H); |
| | | | ¹⁹F NMR (376 MHz, CDCl₃) δ −62.07 |
| J519 | | ESIMS m/z 579 | ¹H NMR (400 MHz, CDCl₃) δ 8.76 (t, J = 1.5 Hz, 1H), 8.44 (d, J = |
| | | ([M + H]⁺) | 1.3 Hz, 1H), 8.07 (dd, J = 8.7, 2.4 Hz, 1H), 7.91 (d, J = 1.4 |
| | | | Hz, 1H), 7.85-7.70 (m, 2H), 7.58-7.53 (m, 2H), 7.50 (d, J = |
| | | | 8.6 Hz, 1H), 7.41-7.30 (m, 3H), 6.92 (t, J = 1.2 Hz, 1H), 3.97 |
| | | | (d, J = 3.0 Hz, 2H), 2.68 (p, J = 6.8 Hz, 1H), 2.39 (s, 3H), 1.19 |
| | | | (t, J = 6.8 Hz, 6H); |
| | | | ¹⁹F NMR (376 MHz, CDCl₃) δ −62.07 |
| J520 | | ESIMS m/z 608 | ¹H NMR (400 MHz, CDCl₃) δ 8.79-8.73 (m, 1H), 8.44 (d, J = |
| | | ([M + H]⁺) | 1.3 Hz, 1H), 8.07 (dd, J = 8.7, 2.4 Hz, 1H), 7.90 (d, J = 1.3 Hz, |
| | | | 1H), 7.83-7.75 (m, 2H), 7.60-7.53 (m, 2H), 7.50 (d, J = 9.8 |
| | | | Hz, 2H), 7.33 (d, J = 8.8 Hz, 1H), 6.89 (dd, J = 8.8, 2.7 Hz, 1H), |
| | | | 6.37 (d, J = 2.8 Hz, 1H), 3.97 (d, J = 2.4 Hz, 2H), 2.96 (s, 6H), |
| | | | 2.60 (p, J = 6.8 Hz, 1H), 1.17 (dd, J = 6.9, 5.6 Hz, 6H); |
| | | | ¹⁹F NMR (376 MHz, CDCl₃) δ −62.07 |
| J521 | | ESIMS m/z 649 | ¹H NMR (400 MHz, CDCl₃) δ 8.76 (s, 1H), 8.43 (d, J = 1.3 Hz, |
| | | ([M + H]⁺) | 1H), 8.07 (dd, J = 8.4, 2.6 Hz, 1H), 7.91 (d, J = 1.4 Hz, 1H), |
| | | | 7.79 (d, J = 8.5 Hz, 2H), 7.58-7.52 (m, 2H), 7.50 (d, J = 8.5 |
| | | | Hz, 1H), 7.42 (d, J = 7.9 Hz, 1H), 7.37-7.31 (m, 1H), 7.29 (s, |
| | | | 1H), 7.04 (s, 1H), 4.62 (d, J = 12.3 Hz, 1H), 4.50 (d, J = 12.3 |
| | | | Hz, 1H), 3.94 (d, J = 2.2 Hz, 2H), 3.73 (qd, J = 8.6, 2.1 Hz, 2H), |
| | | | 2.44 (s, 3H); |
| | | | ¹⁹F NMR (376 MHz, CDCl₃) δ −62.07, −73.76 |
| J522 | | ESIMS m/z 649 | ¹H NMR (400 MHz, CDCl₃) δ 8.76 (s, 1H), 8.43 (d, J = 1.3 Hz, |
| | | ([M + H]⁺) | 1H), 8.07 (dd, J = 8.4, 2.6 Hz, 1H), 7.91 (d, J = 1.4 Hz, 1H), |
| | | | 7.79 (d, J = 8.5 Hz, 2H), 7.58-7.52 (m, 2H), 7.50 (d, J = 8.5 |
| | | | Hz, 1H), 7.42 (d, J = 7.9 Hz, 1H), 7.37-7.31 (m, 1H), 7.29 (s, |
| | | | 1H), 7.04 (s, 1H), 4.62 (d, J = 12.3 Hz, 1H), 4.50 (d, J = 12.3 |
| | | | Hz, 1H), 3.94 (d, J = 2.2 Hz, 2H), 3.73 (qd, J = 8.6, 2.1 Hz, 2H), |
| | | | 2.44 (s, 3H); |
| | | | ¹⁹F NMR (376 MHz, CDCl₃) δ −62.07, −64.58 |
| J523 | | ESIMS m/z 592 | ¹H NMR (400 MHz, CDCl₃) δ 8.04 (d, J = 8.5 Hz, 1H), 7.94 (d, |
| | | ([M + H]⁺) | J = 1.4 Hz, 1H), 7.77 (d, J = 8.4 Hz, 2H), 7.71-7.62 (m, 2H), |
| | | | 7.59-7.55 (m, 2H), 7.50-7.43 (m, 1H), 7.38 (d, J = 8.0 Hz, |
| | | | 1H), 7.31 (d, J = 7.7 Hz, 1H), 7.08 (s, 1H), 6.92 (s, 1H), 3.96 (d, |
| | | | J = 1.0 Hz, 2H), 2.68 (p, J = 6.7 Hz, 1H), 2.39 (s, 3H), 2.23 (s, |
| | | | 3H), 1.20 (dd, J = 16.7, 6.8 Hz, 6H); |
| | | | ¹⁹F NMR (376 MHz, CDCl₃) δ −62.45 |
| J524 | | ESIMS m/z 621 | ¹H NMR (400 MHz, CDCl₃) δ 8.05 (d, J = 8.5 Hz, 1H), 7.94 (d, |
| | | ([M + H]⁺) | J = 1.4 Hz, 1H), 7.77 (d, J = 8.3 Hz, 2H), 7.69 (s, 1H), 7.63 (d, |
| | | | J = 8.9 Hz, 1H), 7.59-7.55 (m, 3H), 7.33 (d, J = 8.7 Hz, 1H), |
| | | | 7.14 (s, 1H), 6.88 (dd, J = 8.7, 2.8 Hz, 1H), 6.38 (d, J = 2.8 Hz, |
| | | | 1H), 3.96 (s, 2H), 2.96 (s, 6H), 2.60 (q, J = 6.9 Hz, 1H), 2.24 (s, |
| | | | 3H), 1.18 (dd, J = 15.2, 6.8 Hz, 6H); |
| | | | ¹⁹F NMR (376 MHz, CDCl₃) δ −62.45 |
| J525 | | ESIMS m/z 608 | ¹H NMR (400 MHz, CDCl₃) δ 8.03 (dd, J = 8.5, 2.8 Hz, 1H), |
| | | ([M + H]⁺) | 7.94 (t, J = 1.0 Hz, 1H), 7.77 (d, J = 8.3 Hz, 2H), 7.68 (q, J = |
| | | | 2.6, 1.7 Hz, 1H), 7.65-7.60 (m, 1H), 7.58 (s, 1H), 7.56 (s, 1H), |
| | | | 7.54 (dd, J = 8.0, 2.8 Hz, 1H), 7.50-7.43 (m, 1H), 7.43-7.33 |
| | | | (m, 1H), 7.02 (d, J = 19.7 Hz, 1H), 6.94 (s, 1H), 4.24-4.03 (m, |
| | | | 1H), 3.97 (d, J = 2.6 Hz, 2H), 3.16 (d, J = 38.8 Hz, 3H), 2.42 (d, |
| | | | J = 2.0 Hz, 3H), 2.20 (d, J = 22.7 Hz, 3H), 1.38 (dd, J = 26.5, |
| | | | 6.4 Hz, 3H); |
| | | | ¹⁹F NMR (376 MHz, CDCl₃) δ −62.45 |
| J526 | | ESIMS m/z 662 | ¹H NMR (400 MHz, CDCl₃) δ 8.04 (d, J = 8.5 Hz, 1H), 7.94 (s, |
| | | ([M + H]⁺) | 1H), 7.77 (d, J = 7.9 Hz, 2H), 7.69 (s, 1H), 7.62 (d, J = 9.8 Hz, |
| | | | 1H), 7.57 (d, J = 8.4 Hz, 3H), 7.45 (dd, J = 18.8, 8.0 Hz, 1H), |
| | | | 7.34 (d, J = 7.6 Hz, 1H), 7.04 (d, J = 9.2 Hz, 2H), 4.63 (d, J = |
| | | | 12.5 Hz, 1H), 4.51 (d, J = 12.3 Hz, 1H), 3.96-3.91 (m, 2H), |
| | | | 3.73 (q, J = 8.8 Hz, 2H), 2.44 (s, 3H), 2.23 (s, 3H); |
| | | | ¹⁹F NMR (376 MHz, CDCl₃) δ −62.45, −73.75 |
| J527 | | ESIMS m/z 662 | ¹H NMR (400 MHz, CDCl₃) δ 8.06 (d, J = 8.6 Hz, 1H), 7.94 (d, |
| | | ([M + H]⁺) | J = 1.4 Hz, 1H), 7.77 (d, J = 8.4 Hz, 2H), 7.69 (s, 1H), 7.69- |
| | | | 7.62 (m, 1H), 7.62-7.54 (m, 2H), 7.50-7.42 (m, 1H), 7.03 (d, |
| | | | J = 11.8 Hz, 2H), 6.95 (dd, J = 10.7, 8.4 Hz, 2H), 4.21 (dt, J = |
| | | | 12.1, 6.3 Hz, 2H), 3.91 (d, J = 1.8 Hz, 2H), 2.61-2.48 (m, 2H), |
| | | | 2.38 (s, 3H), 2.25 (s, 3H); |
| | | | ¹⁹F NMR (376 MHz, CDCl₃) δ −62.45, −64.57 |
| J529 | | ESIMS m/z 623 | ¹H NMR (400 MHz, CDCl₃) δ 7.85 (dd, J = 4.4, 1.4 Hz, 1H), |
| | | ([M + H]⁺) | 7.76 (d, J = 8.5 Hz, 2H), 7.55 (d, J = 8.5 Hz, 1H), 7.50-7.44 |
| | | | (m, 4H), 7.40-7.33 (m, 4H), 6.89 (dd, J = 8.8, 2.8 Hz, 1H), |
| | | | 6.37 (d, J = 2.8 Hz, 1H), 3.97 (d, J = 2.4 Hz, 2H), 2.96 (s, 6H), |
| | | | 2.59 (q, J = 6.7 Hz, 1H), 1.17 (dd, J = 6.8, 5.4 Hz, 6H); |
| | | | ¹⁹F NMR (376 MHz, CDCl₃) δ −58.04 |
| J532 | | ESIMS m/z 666 | ¹H NMR (400 MHz, CDCl₃) δ 8.39 (t, J = 8.4 Hz, 1H), 7.93 (s, |
| | | ([M + H]⁺) | 1H), 7.78 (d, J = 8.2 Hz, 2H), 7.57 (d, J = 6.4 Hz, 5H), 7.52- |
| | | | 7.44 (m, 1H), 7.32-7.27 (m, 1H), 7.02 (s, 1H), 6.96 (d, J = 8.4 |
| | | | Hz, 1H), 4.22 (td, J = 6.3, 2.7 Hz, 2H), 3.93 (s, 2H), 2.52 (d, J = |
| | | | 14.7 Hz, 2H), 2.38 (s, 3H); |
| | | | ¹⁹F NMR (376 MHz, CDCl₃) δ −62.48, −64.59, −131.38 |
| J533 | | ESIMS m/z 639 | ¹H NMR (400 MHz, CDCl₃) δ 8.05 (d, J = 8.4 Hz, 1H), 7.85 (d, |
| | | ([M + H]⁺) | J = 1.4 Hz, 1H), 7.68 (s, 1H), 7.61 (d, J = 8.6 Hz, 1H), 7.56- |
| | | | 7.46 (m, 3H), 7.35 (dd, J = 11.2, 8.6 Hz, 3H), 7.15 (s, 1H), 6.79 |
| | | | (dd, J = 8.6, 2.7 Hz, 1H), 6.49 (d, J = 2.6 Hz, 1H), 4.34 (d, J = |
| | | | 12.2 Hz, 1H), 4.21 (d, J = 12.2 Hz, 1H), 4.03-3.85 (m, 2H), |
| | | | 3.24 (s, 3H), 3.00 (s, 6H), 2.24 (s, 3H); |
| | | | ¹⁹F NMR (376 MHz, CDCl₃) δ −58.04 |
| J534 | | HRMS-ESI (m/z) | ¹H NMR (400 MHz, CDCl₃) δ 8.54 (s, 1H), 8.19 (d, J = 8.5 Hz, |
| | | [M + H]⁺ calcd for | 1H), 8.08-7.93 (m, 2H), 7.85-7.72 (m, 2H), 7.40 (dd, J = |
| | | C₃₁H₂₇F₅N₆O₄S, | 17.8, 8.2 Hz, 3H), 7.33 (d, J = 7.9 Hz, 1H), 7.13-6.99 (m, 2H), |
| | | 675.1807; found, | 4.56 (d, J = 12.3 Hz, 1H), 4.46 (d, J = 12.3 Hz, 1H), 4.00-3.85 |
| | | 675.1804 | (m, 2H), 3.53 (t, J = 12.3 Hz, 2H), 2.44 (s, 3H), 2.26 (s, 3H), |
| | | | 1.58 (d, J = 11.3 Hz, 3H); |
| | | | ¹⁹F NMR (376 MHz, CDCl₃) δ −58.02, −97.95 |
| J535 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (d, J = 8.8 Hz, 1H), 7.88 (dd, J = 12.0, 1.8 Hz, 1H), |
| | | C₃₀H₂₄F₆N₆O₄S, | 7.83-7.72 (m, 2H), 7.49 (d, J = 3.1 Hz, 1H), 7.46-7.37 (m, |
| | | 679.1557; found, | 3H), 7.33 (d, J = 7.8 Hz, 1H), 7.03 (s, 1H), 4.55 (d, J = 12.2 Hz, |
| | | 679.1553 | 1H), 4.45 (d, J = 12.2 Hz, 1H), 4.04-3.87 (m, 2H), 3.52 (t, J = |
| | | | 12.3 Hz, 2H), 2.44 (s, 3H), 1.57 (d, J = 11.8 Hz, 3H); |
| | | | ¹⁹F NMR (376 MHz, CDCl₃) δ −58.02, −98.04, −131.25 |
| J536 | | ESIMS m/z 661 | ¹H NMR (500 MHz, CDCl₃) δ 8.68 (s, 1H), 8.53-8.45 (m, 1H), |
| | | ([M + H]⁺) | 8.04 (d, J = 8.6 Hz, 2H), 8.01-7.95 (m, 3H), 7.92-7.87 (m, |
| | | | 1H), 7.53 (dd, J = 8.0, 5.2 Hz, 1H), 7.39 (t, J = 6.3 Hz, 2H), |
| | | | 6.93 (s, 1H), 4.15 (dd, J = 10.7, 6.3 Hz, 1H), 4.03-3.96 (m, |
| | | | 2H), 3.15 (d, J = 27.9 Hz, 3H), 2.43 (s, 3H), 1.37 (dd, J = 20.2, |
| | | | 6.5 Hz, 3H); |
| | | | ¹⁹F NMR (471 MHz, CDCl₃) δ −74.20, −130.95, −131.38 |
| J539 | | HRMS-ESI (m/z) | ¹H NMR (400 MHz, CDCl₃) δ 8.63 (s, 1H), 8.50 (t, J = 8.4 Hz, |
| | | [M + H]⁺ calcd for | 1H), 7.98 (d, J = 8.6 Hz, 1H), 7.95-7.81 (m, 5H), 7.51 (s, 1H), |
| | | C₂₇H₂₂F₆N₆O₃S2, | 7.41 (d, J = 7.8 Hz, 1H), 7.32 (d, J = 7.9 Hz, 1H), 7.00 (d, J = |
| | | 657.1172; found, | 12.8 Hz, 1H), 4.39 (d, J = 12.6 Hz, 1H), 4.29 (d, J = 12.6 Hz, |
| | | 657.1162 | 1H), 4.03-3.88 (m, 2H), 3.25 (d, J = 19.3 Hz, 3H), 2.42 (d, J = |
| | | | 14.6 Hz, 3H) |
| J540 | | HRMS-ESI (m/z) | ¹H NMR (400 MHz, CDCl₃) δ 8.54 (s, 1H), 8.18 (d, J = 8.5 Hz, |
| | | [M + H]⁺ calcd for | 1H), 8.09-8.01 (m, 1H), 7.98 (s, 1H), 7.83-7.74 (m, 2H), |
| | | C₃₁H₂₆F₆N₆O₄S, | 7.46-7.29 (m, 4H), 7.06 (d, J = 15.8 Hz, 2H), 4.48 (d, J = 12.7 |
| | | 693.1713; found, | Hz, 1H), 4.36 (d, J = 12.7 Hz, 1H), 3.93 (s, 2H), 3.57 (td, J = |
| | | 693.1709 | 6.7, 1.9 Hz, 2H), 2.44 (s, 3H), 2.40-2.29 (m, 2H), 2.26 (s, 3H); |
| | | | ¹⁹F NMR (376 MHz, CDCl₃) δ −58.02, −64.62 |
| J541 | | HRMS-ESI (m/z) | ¹H NMR (400 MHz, CDCl₃) δ 8.63 (s, 1H), 8.51 (t, J = 8.4 Hz, |
| | | [M + H]⁺ calcd for | 1H), 7.97 (d, J = 8.6 Hz, 1H), 7.95-7.81 (m, 5H), 7.54 (d, J = |
| | | C₂₈H₂₄F₆N₆O₃S2, | 3.1 Hz, 1H), 7.25 (s, 1H), 7.02-6.88 (m, 2H), 4.02-3.86 (m, |
| | | 671.1328; found, | 4H), 2.37 (s, 3H), 1.72 (h, J = 7.1 Hz, 2H), 0.93 (t, J = 7.4 Hz, |
| | | 671.1316 | 3H); |
| | | | ¹⁹F NMR (376 MHz, CDCl₃) δ −131.15 |
| J543 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 8.01-7.92 (m, 1H), 7.89 (dd, J = 12.0, 1.8 Hz, 1H), 7.82- |
| | | C₃₀H₂₃F₇N₆O₄S, | 7.71 (m, 2H), 7.49 (d, J = 3.0 Hz, 1H), 7.40 (t, J = 8.5 Hz, |
| | | 697.1462; found, | 3H), 7.33 (d, J = 7.8 Hz, 1H), 7.02 (d, J = 1.5 Hz, 1H), 4.47 (d, |
| | | 697.1458 | J = 12.7 Hz, 1H), 4.35 (d, J = 12.7 Hz, 1H), 3.95 (s, 2H), 3.62- |
| | | | 3.45 (m, 2H), 2.44 (s, 3H), 2.40-2.26 (m, 2H); |
| | | | ¹⁹F NMR (376 MHz, CDCl₃) δ −58.02, −64.66, −131.26 |
| J544 | | HRMS-ESI (m/z) | ¹H NMR (400 MHz, CDCl₃) δ 8.63 (s, 1H), 8.50 (t, J = 8.3 Hz, |
| | | [M + H]⁺ calcd for | 1H), 8.04-7.94 (m, 1H), 7.94-7.84 (m, 5H), 7.52 (d, J = 3.1 |
| | | C₂₉H₂₆F₆N₆O₃S2, | Hz, 1H), 7.42 (d, J = 7.8 Hz, 1H), 7.31 (d, J = 8.0 Hz, 1H), 7.01 |
| | | 685.1485; found, | (d, J = 1.7 Hz, 1H), 4.48-4.30 (m, 2H), 3.95 (s, 2H), 3.31 (t, J = |
| | | 685.1477 | 6.8 Hz, 2H), 2.43 (s, 3H), 1.59-1.50 (m, 2H), 0.87 (t, J = 7.4 |
| | | | Hz, 3H); |
| | | | ¹⁹F NMR (376 MHz, CDCl₃) δ −131.01, −131.41 |
| J545 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.96 (d, J = 8.9 Hz, 1H), 7.88 (dd, J = 12.0, 1.9 Hz, 1H), |
| | | C₃₀H₂₆F₄N₆O₄S, | 7.84-7.72 (m, 2H), 7.51 (d, J = 3.1 Hz, 1H), 7.40 (dd, J = |
| | | 643.1745; found, | 11.6, 8.1 Hz, 3H), 7.35-7.28 (m, 1H), 7.01 (d, J = 1.7 Hz, 1H), |
| | | 643.1742 | 4.45-4.32 (m, 2H), 3.95 (s, 2H), 3.31 (t, J = 6.8 Hz, 2H), 2.43 |
| | | | (s, 3H), 1.56-1.47 (m, 2H), 0.87 (t, J = 7.4 Hz, 3H); |
| | | | ¹⁹F NMR (376 MHz, CDCl₃) δ −58.02, −131.12 |
| J546 | | HRMS-ESI (m/z) | ¹H NMR (400 MHz, CDCl₃) δ 8.54 (s, 1H), 8.20 (d, J = 8.5 Hz, |
| | | [M + H]⁺ calcd for | 1H), 8.03 (dd, J = 8.7, 2.0 Hz, 1H), 8.01-7.93 (m, 1H), 7.84- |
| | | C₃₁H₂₉F₃N₆O₄S, | 7.73 (m, 2H), 7.49-7.34 (m, 3H), 7.30 (d, J = 7.9 Hz, 1H), |
| | | 639.1996; found, | 7.10 (s, 1H), 7.03 (d, J = 1.6 Hz, 1H), 4.39 (q, J = 12.6 Hz, 2H), |
| | | 639.1997 | 3.93 (s, 2H), 3.33 (t, J = 6.8 Hz, 2H), 2.43 (s, 3H), 2.26 (s, 3H), |
| | | | 1.57-1.51 (m, 2H), 0.88 (t, J = 7.4 Hz, 3H); |
| | | | ¹⁹F NMR (376 MHz, CDCl₃) δ −58.02 |
| J547 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 7.98 (d, J = 8.5 Hz, 1H), 7.94-7.85 (m, 3H), 7.80 (d, J = |
| | | C₃₀H₂₃F₇N₆O₃S, | 8.6 Hz, 2H), 7.51 (d, J = 3.0 Hz, 1H), 7.41 (d, J = 7.8 Hz, 1H), |
| | | 681.1513; found, | 7.36-7.30 (m, 1H), 7.02 (s, 1H), 4.47 (d, J = 12.7 Hz, 1H), |
| | | 681.1511 | 4.35 (d, J = 12.7 Hz, 1H), 3.96 (s, 2H), 3.61-3.45 (m, 2H), |
| | | | 2.44 (s, 3H), 2.39-2.26 (m, 2H); |
| | | | ¹⁹F NMR (376 MHz, CDCl₃) δ −62.50, −64.66, −131.19 |
| J548 | | HRMS-ESI (m/z) | ¹H NMR (400 MHz, CDCl₃) δ 8.63 (s, 1H), 8.22 (d, J = 8.5 Hz, |
| | | [M + H]⁺ calcd for | 1H), 8.04 (d, J = 8.5 Hz, 1H), 7.99 (s, 1H), 7.90 (q, J = 9.2 Hz, |
| | | C₂₉H₂₇F₅N₆O₃S2, | 3H), 7.42 (d, J = 7.8 Hz, 1H), 7.30 (d, J = 7.7 Hz, 2H), 7.06 (d, |
| | | 667.1579; found, | J = 22.6 Hz, 2H), 4.49-4.30 (m, 2H), 3.94 (s, 2H), 3.43 (q, J = |
| | | 667.1574 | 7.0 Hz, 2H), 2.43 (s, 3H), 2.27 (s, 3H), 1.17 (t, J = 7.0 Hz, 3H) |
| J549 | | HRMS-ESI (m/z) | ¹H NMR (400 MHz, CDCl₃) δ 8.63 (s, 1H), 8.20 (d, J = 8.5 Hz, |
| | | [M + H]⁺ calcd for | 1H), 8.04 (d, J = 8.7 Hz, 1H), 7.99 (s, 1H), 7.90 (q, J = 9.2 Hz, |
| | | C₂₉H₂₄F₈N₆O₃S2, | 4H), 7.43 (d, J = 7.8 Hz, 1H), 7.34 (d, J = 7.9 Hz, 1H), 7.05 (s, |
| | | 721.1296; found, | 2H), 4.63 (d, J = 12.4 Hz, 1H), 4.51 (d, J = 12.4 Hz, 1H), 3.95 |
| | | 721.1293 | (d, J = 2.1 Hz, 2H), 3.73 (q, J = 8.7 Hz, 2H), 2.45 (s, 3H), 2.27 |
| | | | (s, 3H) |
| J550 | | HRMS-ESI (m/z) | ¹H NMR (400 MHz, CDCl₃) δ 8.63 (s, 1H), 8.48 (t, J = 8.3 Hz, |
| | | [M + H]⁺ calcd for | 1H), 8.05-7.94 (m, 1H), 7.94-7.85 (m, 3H), 7.80 (d, J = 8.5 |
| | | C₂₉H₂₁Cl3F₄N₆O₃S, | Hz, 2H), 7.49 (d, J = 7.8 Hz, 2H), 7.36 (d, J = 8.1 Hz, 1H), 7.05 |
| | | 715.047; found, | (d, J = 1.7 Hz, 1H), 4.77 (d, J = 12.5 Hz, 1H), 4.68 (d, J = 12.5 |
| | | 715.0461 | Hz, 1H), 4.07-3.86 (m, 4H), 2.46 (s, 3H); |
| | | | ¹⁹F NMR (376 MHz, CDCl₃) δ −62.50, −131.15 |
| J551 | | HRMS-ESI (m/z) | ¹H NMR (400 MHz, CDCl₃) δ 8.54 (s, 1H), 8.49 (t, J = 8.4 Hz, |
| | | [M + H]⁺ calcd for | 1H), 7.97 (d, J = 8.9 Hz, 1H), 7.88 (dd, J = 12.1, 1.9 Hz, 1H), |
| | | C₂₉H₂₃F₅N₆O₄S, | 7.82-7.72 (m, 2H), 7.49 (d, J = 3.0 Hz, 1H), 7.40 (t, J = 8.5 |
| | | 647.1494; found, | Hz, 3H), 7.32 (dd, J = 7.9, 1.7 Hz, 1H), 7.03 (s, 1H), 4.63- |
| | | 647.1487 | 4.51 (m, 2H), 4.49-4.39 (m, 2H), 4.02 (d, J = 18.0 Hz, 1H), |
| | | | 3.92 (d, J = 18.0 Hz, 1H), 3.70-3.49 (m, 2H), 2.44 (s, 3H) |
| J552 | | HRMS-ESI (m/z) | ¹H NMR (400 MHz, CDCl₃) δ 8.63 (s, 1H), 8.50 (t, J = 8.4 Hz, |
| | | [M + H]⁺ calcd for | 1H), 7.97 (d, J = 8.6 Hz, 1H), 7.95-7.81 (m, 4H), 7.51 (d, J = |
| | | C₂₈H₂₃F₇N₆O₃S2, | 3.0 Hz, 1H), 7.41 (d, J = 7.8 Hz, 1H), 7.32 (d, J = 8.0 Hz, 1H), |
| | | 689.1234; found, | 7.03 (d, J = 1.5 Hz, 1H), 4.60-4.51 (m, 2H), 4.48-4.38 (m, |
| | | 689.1228 | 2H), 4.16-4.08 (m, 1H), 4.02 (d, J = 18.0 Hz, 1H), 3.92 (d, J = |
| | | | 18.0 Hz, 1H), 3.71-3.48 (m, 2H), 2.44 (s, 3H) |
| J553 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 8.03-7.92 (m, 1H), 7.88 (dd, J = 12.1, 1.9 Hz, 1H), 7.83- |
| | | C₂₉H₂₂Cl2F₄N₆O₄S, | 7.70 (m, 2H), 7.50 (d, J = 3.0 Hz, 1H), 7.47-7.31 (m, 4H), |
| | | 697.0809; found, | 7.03 (d, J = 1.3 Hz, 1H), 5.66 (t, J = 6.0 Hz, 1H), 4.60 (d, J = |
| | | 697.0802 | 12.5 Hz, 1H), 4.48 (d, J = 12.5 Hz, 1H), 4.08-3.87 (m, 2H), |
| | | | 3.82-3.66 (m, 2H), 2.45 (s, 3H); |
| | | | ¹⁹F NMR (376 MHz, CDCl₃) δ −58.02, −131.18 |
| J554 | | HRMS-ESI (m/z) | ¹H NMR (400 MHz, CDCl₃) δ 8.63 (s, 1H), 8.49 (t, J = 8.4 Hz, |
| | | [M + H]⁺ calcd for | 1H), 8.04-7.95 (m, 1H), 7.95-7.83 (m, 5H), 7.50 (d, J = 3.0 |
| | | C₂₈H₂₂Cl2F₆N₆O₃S2, | Hz, 1H), 7.43 (d, J = 7.8 Hz, 1H), 7.34 (d, J = 7.4 Hz, 1H), 7.03 |
| | | 739.0549; found, | (s, 1H), 5.66 (t, J = 6.0 Hz, 1H), 4.60 (d, J = 12.5 Hz, 1H), 4.48 |
| | | 739.0535 | (d, J = 12.5 Hz, 1H), 4.08-3.89 (m, 2H), 3.82-3.66 (m, 2H), |
| | | | 2.45 (s, 3H) |
| J555 | | HRMS-ESI (m/z) | ¹H NMR (400 MHz, CDCl₃) δ 8.54 (s, 1H), 8.19 (d, J = 8.5 Hz, |
| | | [M + H]⁺ calcd for | 1H), 8.03 (d, J = 8.8 Hz, 1H), 7.98 (s, 1H), 7.85-7.73 (m, 2H), |
| | | C₃₀H₂₅Cl2F₃N₆O₄S, | 7.44 (d, J = 7.8 Hz, 1H), 7.36 (dd, J = 18.9, 8.2 Hz, 3H), 7.12- |
| | | 693.106; found, | 7.01 (m, 2H), 5.68 (t, J = 6.0 Hz, 1H), 4.60 (d, J = 12.6 Hz, 1H), |
| | | 693.1049 | 4.50 (d, J = 12.5 Hz, 1H), 4.04-3.88 (m, 2H), 3.76 (d, J = 5.9 |
| | | | Hz, 2H), 2.44 (s, 3H), 2.27 (s, 3H); |
| | | | ¹⁹F NMR (376 MHz, CDCl₃) δ −58.02 |
| J556 | | HRMS-ESI (m/z) | ¹H NMR (400 MHz, CDCl₃) δ 8.63 (s, 1H), 8.20 (d, J = 8.5 Hz, |
| | | [M + H]⁺ calcd for | 1H), 8.04 (d, J = 9.0 Hz, 1H), 7.99 (s, 1H), 7.90 (q, J = 9.2 Hz, |
| | | C₂₉H₂₅Cl2F₅N₆O₃S2, | 4H), 7.44 (d, J = 7.8 Hz, 1H), 7.34 (d, J = 7.8 Hz, 1H), 7.06 (d, |
| | | 735.08; found, | J = 10.2 Hz, 2H), 5.68 (t, J = 5.9 Hz, 1H), 4.60 (d, J = 12.6 Hz, |
| | | 735.0791 | 1H), 4.50 (d, J = 12.6 Hz, 1H), 4.04-3.88 (m, 2H), 3.76 (d, J = |
| | | | 5.9 Hz, 2H), 2.45 (s, 3H), 2.27 (s, 3H) |
| J557 | | HRMS-ESI (m/z) | ¹H NMR (400 MHz, CDCl₃) δ 8.63 (s, 1H), 8.22 (d, J = 8.5 Hz, |
| | | [M + H]⁺ calcd for | 1H), 8.04 (d, J = 8.6 Hz, 1H), 7.99 (s, 1H), 7.90 (q, J = 9.2 Hz, |
| | | C₂₉H₂₆F₆N₆O₃S2, | 4H), 7.42 (d, J = 7.8 Hz, 1H), 7.31 (d, J = 7.9 Hz, 1H), 7.05 (d, |
| | | 685.1485; found, | J = 10.4 Hz, 2H), 4.63-4.48 (m, 2H), 4.44 (d, J = 12.0 Hz, |
| | | 685.1474 | 2H), 3.95 (q, J = 18.0 Hz, 2H), 3.72-3.51 (m, 2H), 2.44 (s, |
| | | | 3H), 2.27 (s, 3H) |
| J558 | | HRMS-ESI (m/z) | ¹H NMR (400 MHz, CDCl₃) δ 8.53 (s, 1H), 8.20 (d, J = 8.6 Hz, |
| | | [M + H]⁺ calcd for | 1H), 8.03 (d, J = 8.5 Hz, 1H), 7.98 (s, 1H), 7.84-7.73 (m, 2H), |
| | | C₃₀H₂₆F₄N₆O₄S, | 7.40 (dd, J = 16.7, 8.2 Hz, 3H), 7.31 (d, J = 7.9 Hz, 1H), 7.05 |
| | | 643.1745; found, | (d, J = 8.0 Hz, 2H), 4.61-4.50 (m, 2H), 4.44 (d, J = 12.1 Hz, |
| | | 643.1735 | 2H), 3.95 (q, J = 18.0 Hz, 2H), 3.70-3.52 (m, 2H), 2.43 (s, |
| | | | 3H), 2.26 (s, 3H) |
| J559 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 8.04-7.93 (m, 1H), 7.93-7.84 (m, 3H), 7.80 (d, J = 8.6 |
| | | C₂₉H₂₂Cl2F₄N₆O₃S, | Hz, 2H), 7.50 (d, J = 3.0 Hz, 1H), 7.43 (d, J = 7.8 Hz, 1H), 7.38- |
| | | 681.086; found, | 7.31 (m, 1H), 7.03 (d, J = 1.5 Hz, 1H), 5.66 (t, J = 6.0 Hz, |
| | | 681.0848 | 1H), 4.60 (d, J = 12.5 Hz, 1H), 4.48 (d, J = 12.6 Hz, 1H), 4.06- |
| | | | 3.91 (m, 2H), 3.74 (d, J = 5.9 Hz, 2H), 2.45 (s, 3H); |
| | | | ¹⁹F NMR (376 MHz, CDCl₃) δ −62.52, −131.14 |
| J560 | | HRMS-ESI (m/z) | ¹H NMR (400 MHz, CDCl₃) δ 8.54 (s, 1H), 8.48 (t, J = 8.3 Hz, |
| | | [M + H]⁺ calcd for | 1H), 7.97 (d, J = 9.0 Hz, 1H), 7.89 (dd, J = 12.0, 1.8 Hz, 1H), |
| | | C₃₀H₂₃F₇N₆O₆S, | 7.82-7.75 (m, 2H), 7.50 (d, J = 3.0 Hz, 1H), 7.44-7.31 (m, |
| | | 729.1361; found, | 2H), 7.02 (s, 1H), 6.68 (s, 1H), 4.55 (d, J = 12.4 Hz, 1H), 4.47 |
| | | 729.135 | (d, J = 12.4 Hz, 1H), 3.97 (d, J = 4.3 Hz, 5H), 3.91 (s, 3H), 3.75 |
| | | | (qd, J = 8.7, 2.5 Hz, 2H); |
| | | | ¹⁹F NMR (376 MHz, CDCl₃) δ −58.03, −73.74, −131.30 |
| J561 | | HRMS-ESI (m/z) | ¹H NMR (400 MHz, CDCl₃) δ 8.63 (s, 1H), 8.49 (t, J = 8.4 Hz, |
| | | [M + H]⁺ calcd for | 1H), 7.98 (dd, J = 8.6, 1.6 Hz, 1H), 7.95-7.82 (m, 5H), 7.51 |
| | | C₂₉H₂₃F₉N₆O₅S2, | (d, J = 3.1 Hz, 1H), 7.02 (s, 1H), 6.68 (s, 1H), 4.56 (d, J = 12.4 |
| | | 771.11; found, | Hz, 1H), 4.47 (d, J = 12.4 Hz, 1H), 3.97 (d, J = 5.4 Hz, 5H), |
| | | 771.1092 | 3.91 (s, 3H), 3.75 (qd, J = 8.7, 2.4 Hz, 2H) |
| J562 | | ESIMS m/z 645 | ¹H NMR (400 MHz, DMSO-d₆) δ 9.54 (s, 1H), 9.47 (s, 1H), 8.19 |
| | | ([M + H]⁺) | (d, J = 8.4 Hz, 2H), 7.99 (d, J = 8.5 Hz, 2H), 7.96-7.79 (m, |
| | | | 3H), 7.24-7.20 (m, 4H), 7.18-7.10 (m, 4H), 4.05-3.93 (m, |
| | | | 2H), 3.78 (s, 2H), 2.32 (s, 3H); |
| | | | ¹⁹F NMR (376 MHz, DMSO-d₆) δ −60.81, −121.83 |
| J563 | | ESIMS m/z 703 | ¹H NMR (400 MHz, DMSO-d₆) δ 9.55 (s, 1H), 9.48 (s, 1H), 8.23- |
| | | ([M + H]⁺) | 8.13 (m, 4H), 7.96-7.78 (m, 3H), 7.24-7.19 (m, 4H), 7.18- |
| | | | 7.10 (m, 4H), 4.08-3.86 (m, 2H), 3.78 (s, 2H), 2.32 (s, 3H) |
| J564 | | HRMS-ESI (m/z) | ¹H NMR (400 MHz, CDCl₃) δ 8.54 (s, 1H), 8.48 (t, J = 8.4 Hz, |
| | | [M + H]⁺ calcd for | 1H), 7.97 (d, J = 8.6 Hz, 1H), 7.89 (dd, J = 12.0, 1.9 Hz, 1H), |
| | | C₂₉H₂₁F₇N₆O₅S, | 7.83-7.72 (m, 2H), 7.48 (d, J = 3.1 Hz, 1H), 7.46-7.34 (m, |
| | | 699.1255; found, | 2H), 7.05 (td, J = 8.2, 2.5 Hz, 1H), 6.81-6.72 (m, 1H), 4.59 (d, |
| | | 699.125 | J = 12.3 Hz, 1H), 4.47 (d, J = 12.3 Hz, 1H), 3.96 (d, J = 3.1 Hz, |
| | | | 2H), 3.87 (s, 3H), 3.69 (pq, J = 8.6, 4.8, 4.2 Hz, 3H); |
| | | | ¹⁹F NMR (376 MHz, CDCl₃) δ −58.03, −73.73, −131.33 |
| J565 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 8.04-7.96 (m, 1H), 7.97-7.89 (m, 3H), 7.87 (d, J = 9.1 |
| | | C₂₈H₂₁F₉N₆O₄S2, | Hz, 2H), 7.49 (d, J = 3.0 Hz, 1H), 7.43 (d, J = 8.5 Hz, 1H), 7.06 |
| | | 741.0995; found, | (dd, J = 8.5, 2.6 Hz, 1H), 6.76 (d, J = 2.6 Hz, 1H), 4.60 (d, J = |
| | | 741.0989 | 12.3 Hz, 1H), 4.47 (d, J = 12.3 Hz, 1H), 3.96 (d, J = 3.1 Hz, |
| | | | 2H), 3.87 (s, 3H), 3.70 (qd, J = 8.8, 4.8 Hz, 2H) |
| J566 | | HRMS-ESI (m/z) | ¹H NMR (400 MHz, CDCl₃) δ 8.54 (s, 1H), 8.48 (t, J = 8.3 Hz, |
| | | [M + H]⁺ calcd for | 1H), 7.97 (d, J = 8.6 Hz, 1H), 7.88 (dd, J = 12.1, 1.8 Hz, 1H), |
| | | C₂₉H₂₁F₇N₆O₄S, | 7.82-7.72 (m, 2H), 7.47 (d, J = 6.1 Hz, 1H), 7.41 (dd, J = |
| | | 683.1306; found, | 18.1, 8.6 Hz, 4H), 7.05 (d, J = 7.6 Hz, 1H), 4.60 (d, J = 3.2 Hz, |
| | | 683.1294 | 2H), 3.97 (d, J = 1.5 Hz, 2H), 3.81-3.64 (m, 2H), 2.51 (s, 3H); |
| | | | ¹⁹F NMR (376 MHz, CDCl₃) δ −58.03, −73.65, −131.32 |
| J567 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 7.98 (d, J = 8.6 Hz, 1H), 7.95-7.89 (m, 2H), 7.87 (d, J = |
| | | C₂₈H₂₁F₉N₆O₃S2, | 8.9 Hz, 3H), 7.48 (d, J = 11.9 Hz, 1H), 7.41 (dd, J = 14.9, 7.6 |
| | | 725.1046; found, | Hz, 2H), 7.05 (d, J = 7.6 Hz, 1H), 4.60 (d, J = 2.8 Hz, 2H), 3.97 |
| | | 725.1043 | (d, J = 1.5 Hz, 2H), 3.74 (qd, J = 8.9, 6.0 Hz, 2H), 2.49 (d, J = |
| | | | 19.9 Hz, 3H); |
| | | | ¹⁹F NMR (376 MHz, CDCl₃) δ −73.65, −131.19 |
| J568 | | ESIMS m/z 708 | ¹H NMR (500 MHz, CDCl₃) δ 8.54 (s, 1H), 8.46 (t, J = 8.4 Hz, |
| | | ([M − H]⁻) | 1H), 7.96 (dd, J = 8.6, 1.8 Hz, 1H), 7.87 (dd, J = 12.0, 1.8 Hz, |
| | | | 1H), 7.82-7.75 (m, 2H), 7.52 (d, J = 3.0 Hz, 1H), 7.45-7.32 |
| | | | (m, 4H), 7.26-7.20 (m, 1H), 7.09-6.99 (m, 3H), 6.91 (td, J = |
| | | | 8.5, 2.6 Hz, 1H), 4.50 (d, J = 12.4 Hz, 1H), 4.47-4.37 (m, 3H), |
| | | | 3.86 (s, 1H), 3.75 (d, J = 18.0 Hz, 1H), 2.44 (s, 3H); |
| | | | ¹³C NMR (126 MHz, CDCl₃) δ 172.28, 170.86, 163.84, 162.29, |
| | | | 162.27, 161.88, 159.37, 152.95, 151.02, 148.45, 148.43, |
| | | | 141.61, 140.46, 140.40, 139.73, 135.44, 133.72, 132.23, |
| | | | 130.80, 130.18, 129.95, 129.93, 129.88, 129.58, 127.88, |
| | | | 127.80, 126.17, 126.11, 123.11, 122.90, 122.88, 122.87, |
| | | | 122.45, 121.42, 121.21, 120.22, 119.36, 117.31, 114.60, |
| | | | 114.55, 114.44, 114.42, 114.36, 114.19, 113.06, 112.89, |
| | | | 71.40, 71.39, 69.44, 33.02, 21.13, 21.07; |
| | | | ¹⁹F NMR (471 MHz, CDCl₃) δ −58.00, −112.98, −131.12 |
| J569 | | ESIMS m/z 732 | ¹H NMR (400 MHz, CDCl₃) δ 8.65 (s, 1H), 8.62 (d, J = 8.8 Hz, |
| | | ([M + H]⁺) | 1H), 8.45-8.32 (m, 2H), 7.98-7.91 (m, 2H), 7.87 (d, J = 8.9 |
| | | | Hz, 2H), 7.73 (s, 1H), 7.40 (d, J = 7.8 Hz, 1H), 7.37-7.31 (m, |
| | | | 1H), 7.04 (d, J = 1.5 Hz, 1H), 4.63 (d, J = 12.4 Hz, 1H), 4.50 (d, |
| | | | J = 12.4 Hz, 1H), 3.97 (d, J = 3.2 Hz, 2H), 3.72 (qt, J = 8.7, 4.2 |
| | | | Hz, 2H), 2.46 (s, 3H) |
| J570 | | ESIMS m/z 774 | ¹H NMR (500 MHz, CDCl₃) δ 8.57-8.43 (m, 1H), 7.96 (dd, J = |
| | | ([M − H]⁻) | 8.5, 1.8 Hz, 1H), 7.88 (dd, J = 12.0, 1.8 Hz, 1H), 7.85-7.73 |
| | | | (m, 2H), 7.54 (d, J = 3.0 Hz, 1H), 7.44-7.26 (m, 6H), 7.17 (dd, |
| | | | J = 14.7, 8.1 Hz, 2H), 7.03 (t, J = 2.4 Hz, 1H), 4.56-4.32 (m, |
| | | | 4H), 4.00-3.62 (m, 2H), 2.42 (d, J = 23.9 Hz, 3H); |
| | | | ¹³C NMR (126 MHz, CDCl₃) δ 172.28, 170.83, 162.28, 159.38, |
| | | | 152.96, 151.03, 148.65, 148.45, 141.62, 141.36, 139.76, |
| | | | 136.73, 136.53, 135.59, 135.45, 133.71, 132.21, 130.95, |
| | | | 130.82, 130.31, 130.17, 129.89, 129.58, 129.15, 129.00, |
| | | | 127.82, 127.74, 126.24, 126.18, 123.05, 122.92, 122.45, |
| | | | 122.42, 121.46, 121.42, 121.22, 121.08, 120.93, 120.21, |
| | | | 119.37, 116.59, 113.74, 113.58, 113.09, 112.92, 71.35, 69.50, |
| | | | 69.02, 32.98, 29.74, 21.14, 21.08 |
| J571 | | ESIMS m/z 689 | ¹H NMR (400 MHz, CDCl₃) δ 8.54 (s, 1H), 8.47 (t, J = 8.4 Hz, |
| | | ([M − H]⁻) | 1H), 7.97 (d, J = 8.5 Hz, 1H), 7.88 (dd, J = 12.0, 1.8 Hz, 1H), |
| | | | 7.82-7.74 (m, 2H), 7.48 (d, J = 3.0 Hz, 1H), 7.41 (dd, J = |
| | | | 17.2, 8.2 Hz, 3H), 7.35-7.27 (m, 5H), 7.02 (d, J = 1.7 Hz, 1H), |
| | | | 4.49 (d, J = 12.3 Hz, 1H), 4.46-4.38 (m, 3H), 4.12 (q, J = 7.1 |
| | | | Hz, 1H), 3.80 (s, 1H), 3.66 (s, 1H), 2.44 (s, 3H); |
| | | | ¹³C NMR (126 MHz, CDCl₃) δ 172.28, 170.86, 162.28, 162.26, |
| | | | 159.41, 152.93, 151.00, 148.42, 141.58, 139.52, 137.70, |
| | | | 135.42, 133.65, 132.45, 130.72, 130.14, 129.80, 129.51, |
| | | | 128.39, 127.91, 127.87, 127.83, 127.73, 127.71, 127.68, |
| | | | 126.11, 126.04, 122.84, 122.42, 121.39, 121.19, 120.18, |
| | | | 119.34, 117.28, 113.05, 112.88, 72.33, 69.29, 32.94, 21.10, |
| | | | 21.04 |
| J572 | | | ¹H NMR (500 MHz, CDCl₃) δ 8.55 (s, 1H), 8.43 (td, J = 8.3, 3.8 |
| | | | Hz, 1H), 7.95 (dd, J = 8.7, 1.9 Hz, 1H), 7.87 (dd, J = 12.0, 1.9 |
| | | | Hz, 1H), 7.82-7.73 (m, 1H), 7.48 (m, 2H), 7.43-7.27 (m, |
| | | | 4H), 7.25-7.10 (m, 3H), 7.03 (d, J = 1.8 Hz, 1H), 4.63-4.44 |
| | | | (m, 5H), 4.07-3.73 (m, 2H), 2.42 (d, J = 24.9 Hz, 3H); |
| | | | ¹³C NMR (126 MHz, CDCl₃) δ 172.24, 170.89, 162.30, 159.31, |
| | | | 152.93, 151.00, 148.43, 141.61, 139.73, 135.81, 135.62, |
| | | | 135.45, 133.68, 132.84, 132.55, 132.32, 130.97, 130.84, |
| | | | 130.24, 129.80, 129.57, 129.22, 129.13, 128.93, 128.75, |
| | | | 128.69, 128.62, 128.48, 127.90, 127.82, 126.81, 126.10, |
| | | | 126.04, 122.82, 122.46, 121.42, 121.21, 120.17, 119.36, |
| | | | 113.05, 112.88, 69.94, 69.50, 69.32, 33.07, 21.15, 21.08; |
| | | | ¹⁹F NMR (471 MHz, CDCl₃) δ −58.00, −130.98 |
| J573 | | | ¹H NMR (500 MHz, CDCl₃) δ 8.55 (s, 1H), 8.46 (t, J = 8.4 Hz, |
| | | | 1H), 7.97 (dd, J = 8.6, 1.8 Hz, 1H), 7.88 (dd, J = 12.0, 1.9 Hz, |
| | | | 1H), 7.84-7.74 (m, 2H), 7.51 (d, J = 3.0 Hz, 1H), 7.45-7.30 |
| | | | (m, 4H), 7.09-6.99 (m, 1H), 6.85-6.77 (m, 1H), 6.73 (ddd, J = |
| | | | 10.1, 8.9, 2.5 Hz, 1H), 4.56-4.35 (m, 4H), 3.97-3.74 (m, |
| | | | 2H), 2.44 (s, 3H); |
| | | | ¹³C NMR (126 MHz, CDCl₃) δ 196.26, 172.25, 170.83, 163.51, |
| | | | 162.28, 161.69, 161.62, 161.53, 159.71, 159.61, 159.34, |
| | | | 152.94, 151.01, 148.44, 141.61, 139.79, 135.45, 133.71, |
| | | | 132.17, 130.83, 130.81, 130.76, 130.73, 130.68, 130.23, |
| | | | 129.57, 127.85, 127.77, 126.20, 126.13, 122.90, 122.46, |
| | | | 121.42, 121.22, 120.86, 120.18, 119.36, 113.08, 112.90, |
| | | | 111.33, 111.30, 111.13, 103.69, 103.49, 69.66, 65.38, 65.36, |
| | | | 35.54, 21.14; |
| | | | ¹⁹F NMR (471 MHz, CDCl₃) δ −58.01, −110.56, −114.45, −131.18 |
| J574 | | ESIMS m/z 707 | ¹H NMR (500 MHz, CDCl₃) δ 8.47 (t, J = 8.4 Hz, 1H), 7.96 (dd, |
| | | ([M-H]-) | J = 8.6, 1.8 Hz, 1H), 7.87 (dd, J = 12.0, 1.9 Hz, 1H), 7.81- |
| | | | 7.75 (m, 2H), 7.53 (d, J = 3.0 Hz, 1H), 7.44-7.30 (m, 4H), |
| | | | 7.24 (d, J = 5.6 Hz, 1H), 7.02 (d, J = 1.7 Hz, 1H), 7.01-6.94 |
| | | | (m, 2H), 4.53-4.30 (m, 4H), 3.91-3.59 (m, 2H), 2.44 (s, 3H); |
| | | | ¹³C NMR (126 MHz, CDCl₃) δ 172.29, 170.84, 163.30, 162.28, |
| | | | 162.26, 161.34, 159.40, 152.95, 151.02, 148.45, 148.44, |
| | | | 141.61, 139.66, 135.45, 133.70, 133.54, 133.51, 132.32, |
| | | | 130.78, 130.16, 129.57, 129.51, 129.45, 127.86, 127.78, |
| | | | 126.21, 126.14, 123.47, 122.93, 122.90, 122.45, 121.42, |
| | | | 121.22, 120.20, 119.36, 115.36, 115.19, 113.08, 112.92, |
| | | | 71.59, 69.39, 33.00, 21.13, 14.22; |
| | | | ¹⁹F NMR (471 MHz, CDCl₃) δ −58.00, −114.53, −131.11 |
| J575 | | HRMS-ESI (m/z) | ¹H NMR (400 MHz, CDCl₃) δ 8.53 (s, 1H), 8.18 (d, J = 8.5 Hz, |
| | | [M + H]⁺ calcd for | 1H), 8.04 (d, J = 8.5 Hz, 1H), 7.99 (s, 1H), 7.83-7.75 (m, 2H), |
| | | C₃₀H₂₄F₆N₆O₅S, | 7.44 (d, J = 8.5 Hz, 1H), 7.38 (d, J = 8.6 Hz, 2H), 7.10-7.01 |
| | | 695.1506; found, | (m, 2H), 6.78 (d, J = 2.6 Hz, 1H), 4.60 (d, J = 12.3 Hz, 1H), |
| | | 695.1503 | 4.48 (d, J = 12.2 Hz, 1H), 3.94 (d, J = 2.6 Hz, 2H), 3.87 (s, 3H), |
| | | | 3.79-3.66 (m, 2H), 2.26 (s, 3H); |
| | | | ¹⁹F NMR (376 MHz, CDCl₃) δ −58.03, −73.71 |
| J576 | | HRMS-ESI (m/z) | ¹H NMR (400 MHz, CDCl₃) δ 8.53 (s, 1H), 8.17 (d, J = 8.6 Hz, |
| | | [M + H]⁺ calcd for | 1H), 8.03 (d, J = 8.9 Hz, 1H), 7.98 (s, 1H), 7.83-7.73 (m, 2H), |
| | | C₃₀H₂₄F₆N₆O₄S, | 7.44 (t, J = 7.7 Hz, 1H), 7.38 (d, J = 8.8 Hz, 3H), 7.06 (d, J = |
| | | 679.1557; found, | 10.4 Hz, 2H), 4.67-4.51 (m, 2H), 3.95 (d, J = 1.6 Hz, 2H), |
| | | 679.1554 | 3.74 (qd, J = 8.7, 3.4 Hz, 2H), 2.51 (s, 3H), 2.25 (s, 3H); |
| | | | ¹⁹F NMR (376 MHz, CDCl₃) δ −58.03, −73.64 |
| J577 | | HRMS-ESI (m/z) | ¹H NMR (400 MHz, CDCl₃) δ 8.63 (s, 1H), 8.20 (d, J = 8.5 Hz, |
| | | [M + H]⁺ calcd for | 1H), 8.04 (d, J = 8.9 Hz, 1H), 7.99 (s, 1H), 7.90 (q, J = 9.2 Hz, |
| | | C₂₉H₂₄F₈N₆O₄S2, | 4H), 7.44 (d, J = 8.5 Hz, 1H), 7.06 (dd, J = 8.5, 2.4 Hz, 2H), |
| | | 737.1245; found, | 6.84-6.72 (m, 1H), 4.60 (d, J = 12.3 Hz, 1H), 4.54-4.39 (m, |
| | | 737.1244 | 1H), 3.94 (d, J = 2.7 Hz, 2H), 3.85 (d, J = 10.5 Hz, 3H), 3.78- |
| | | | 3.65 (m, 2H), 2.27 (s, 3H) |
| J578 | | HRMS-ESI (m/z) | ¹H NMR (400 MHz, CDCl₃) δ 8.62 (s, 1H), 8.18 (d, J = 8.5 Hz, |
| | | [M + H]⁺ calcd for | 1H), 8.07-8.01 (m, 1H), 7.98 (s, 1H), 7.89 (q, J = 9.2 Hz, 4H), |
| | | C₂₉H₂₄F₈N₆O₃S2, | 7.49-7.33 (m, 2H), 7.06 (d, J = 5.5 Hz, 2H), 4.69-4.49 (m, |
| | | 721.1296; found, | 2H), 3.96 (d, J = 1.6 Hz, 2H), 3.74 (qd, J = 8.8, 3.4 Hz, 2H), |
| | | 721.1296 | 2.51 (s, 3H), 2.25 (s, 3H) |
| J579 | | HRMS-ESI (m/z) | ¹H NMR (400 MHz, CDCl₃) δ 8.63 (s, 1H), 8.18 (d, J = 8.5 Hz, |
| | | [M + H]⁺ calcd for | 1H), 8.05 (d, J = 8.7 Hz, 1H), 7.99 (s, 1H), 7.90 (d, J = 8.4 Hz, |
| | | C₃₀H₂₄F₆N₆O₃S, | 2H), 7.79 (d, J = 8.5 Hz, 2H), 7.49-7.35 (m, 2H), 7.06 (d, J = |
| | | 663.1608; found, | 9.2 Hz, 2H), 4.67-4.52 (m, 2H), 3.96 (d, J = 1.6 Hz, 2H), 3.74 |
| | | 663.1599 | (qd, J = 8.7, 3.4 Hz, 2H), 2.51 (s, 3H), 2.26 (s, 3H); |
| | | | ¹⁹F NMR (376 MHz, CDCl₃) δ −62.49, −73.64 |
| J580 | | HRMS-ESI (m/z) | ¹H NMR (400 MHz, CDCl₃) δ 8.63 (s, 1H), 8.20 (d, J = 8.5 Hz, |
| | | [M + H]⁺ calcd for | 1H), 8.05 (d, J = 9.1 Hz, 1H), 8.00 (s, 1H), 7.90 (d, J = 8.5 Hz, |
| | | C₃₀H₂₄F₆N₆O₄S, | 2H), 7.79 (d, J = 8.5 Hz, 2H), 7.44 (d, J = 8.5 Hz, 1H), 7.11- |
| | | 679.1557; found, | 7.01 (m, 2H), 6.78 (d, J = 2.6 Hz, 1H), 4.60 (d, J = 12.3 Hz, |
| | | 679.155 | 1H), 4.48 (d, J = 12.3 Hz, 1H), 3.94 (d, J = 2.7 Hz, 2H), 3.87 (s, |
| | | | 3H), 3.78-3.63 (m, 2H), 2.27 (s, 3H); |
| | | | ¹⁹F NMR (376 MHz, CDCl₃) δ −62.49, −73.71 |
| J581 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 7.98 (d, J = 8.6 Hz, 1H), 7.94-7.85 (m, 3H), 7.80 (d, J = |
| | | C₂₉H₂₁F₇N₆O₄S, | 8.5 Hz, 2H), 7.49 (d, J = 2.9 Hz, 1H), 7.43 (d, J = 8.5 Hz, 1H), |
| | | 683.1306; found, | 7.06 (dd, J = 8.5, 2.6 Hz, 1H), 6.76 (d, J = 2.6 Hz, 1H), 4.60 (d, |
| | | 683.1304 | J = 12.3 Hz, 1H), 4.47 (d, J = 12.3 Hz, 1H), 3.96 (d, J = 3.1 Hz, |
| | | | 2H), 3.87 (s, 3H), 3.70 (qt, J = 8.3, 4.3 Hz, 2H); |
| | | | ¹⁹F NMR (376 MHz, CDCl₃) δ −62.52, −73.73, −131.27 |
| J582 | 223-227 | ESIMS m/z 678 | ¹H NMR (400 MHz, CDCl₃) δ 8.65 (s, 1H), 8.62 (d, J = 8.8 Hz, |
| | | ([M + H]⁺) | 1H), 8.41-8.34 (m, 2H), 7.98-7.90 (m, 2H), 7.87 (d, J = 8.9 |
| | | | Hz, 2H), 7.76 (s, 1H), 7.38 (d, J = 7.8 Hz, 1H), 7.33-7.27 (m, |
| | | | 1H), 7.01 (d, J = 1.5 Hz, 1H), 4.44 (d, J = 12.6 Hz, 1H), 4.36 (d, |
| | | | J = 12.6 Hz, 1H), 3.96 (d, J = 1.1 Hz, 2H), 3.40 (q, J = 7.0 Hz, |
| | | | 2H), 2.43 (s, 3H), 1.14 (t, J = 7.0 Hz, 3H) |
| J583 | 195-198 | ESIMS m/z 662 | ¹H NMR (400 MHz, CDCl₃) δ 8.65 (s, 1H), 8.61 (d, J = 8.4 Hz, |
| | | ([M + H]⁺) | 1H), 8.42-8.35 (m, 2H), 7.97-7.91 (m, 2H), 7.87 (d, J = 8.8 |
| | | | Hz, 2H), 7.79 (s, 1H), 7.34-7.25 (m, 2H), 6.91 (d, J = 1.7 Hz, |
| | | | 1H), 4.00 (d, J = 2.0 Hz, 2H), 2.42-2.33 (m, 5H), 1.61 (dt, J = |
| | | | 15.5, 7.7 Hz, 2H), 0.93 (t, J = 7.4 Hz, 3H) |
| J584 | | ESIMS m/z 732 | ¹H NMR (400 MHz, CDCl₃) δ 8.67-8.60 (m, 2H), 8.41-8.34 |
| | | ([M + H]⁺) | (m, 2H), 7.98-7.90 (m, 2H), 7.87 (d, J = 8.9 Hz, 2H), 7.76 (s, |
| | | | 1H), 7.28 (dd, J = 8.4, 2.2 Hz, 1H), 7.01 (d, J = 2.2 Hz, 1H), |
| | | | 6.97 (d, J = 8.5 Hz, 1H), 4.31-4.17 (m, 2H), 3.95 (d, J = 1.2 |
| | | | Hz, 2H), 2.62-2.47 (m, 2H), 2.38 (s, 3H) |
| J585 | | ESIMS m/z 691 | ¹H NMR (400 MHz, DMSO-d₆) δ 9.98 (s, 1H), 9.58 (s, 1H), 8.33 |
| | | ([M + H]⁺) | (s, 2H), 8.24-8.12 (m, 4H), 7.72 (s, 1H), 7.27 (s, 1H), 6.82 (s, |
| | | | 1H), 6.60 (s, 1H), 4.22 (d, J = 18.1 Hz, 1H), 4.09 (d, J = 18.0 |
| | | | Hz, 1H), 2.88 (s, 6H), 2.63-2.52 (m, 1H), 1.17 (dd, J = 14.1, |
| | | | 7.0 Hz, 3H), 1.06 (d, J = 6.9 Hz, 3H) |
| J586 | | ESIMS m/z 692 | ¹H NMR (400 MHz, CDCl₃) δ 8.65 (s, 1H), 8.61 (d, J = 9.0 Hz, |
| | | ([M + H]⁺) | 1H), 8.44-8.34 (m, 2H), 7.98-7.91 (m, 2H), 7.87 (d, J = 9.0 |
| | | | Hz, 2H), 7.73 (d, J = 18.9 Hz, 1H), 7.55 (dd, J = 8.0, 2.7 Hz, |
| | | | 1H), 7.37 (dd, J = 8.2, 1.7 Hz, 1H), 6.90 (d, J = 1.9 Hz, 1H), |
| | | | 4.28 (q, J = 6.4 Hz, 1H), 4.00 (d, J = 1.2 Hz, 2H), 3.49-3.37 |
| | | | (m, 1H), 3.34-3.22 (m, 1H), 2.42 (s, 3H), 1.34 (d, J = 6.5 Hz, |
| | | | 3H), 1.18 (t, J = 7.0 Hz, 3H) |
| J587 | | ESIMS m/z 746 | ¹H NMR (400 MHz, CDCl₃) δ 8.66-8.61 (m, 2H), 8.41-8.35 |
| | | ([M + H]⁺) | (m, 2H), 7.98-7.90 (m, 2H), 7.87 (d, J = 8.9 Hz, 2H), 7.73 (s, |
| | | | 1H), 7.26 (s, 1H), 6.98 (s, 1H), 4.60 (d, J = 12.4 Hz, 1H), 4.47 |
| | | | (d, J = 12.4 Hz, 1H), 3.96 (d, J = 2.8 Hz, 2H), 3.71 (qd, J = 8.7, |
| | | | 4.0 Hz, 2H), 2.36 (s, 3H), 2.34 (s, 3H) |
| J588 | | ESIMS m/z 778 | ¹H NMR (400 MHz, CDCl₃) δ 8.66 (s, 1H), 8.61 (d, J = 8.9 Hz, |
| | | ([M + H]⁺) | 1H), 8.42-8.35 (m, 2H), 7.99-7.90 (m, 2H), 7.88 (d, J = 8.8 |
| | | | Hz, 2H), 7.77 (s, 1H), 7.00 (s, 1H), 6.68 (s, 1H), 4.56 (d, J = |
| | | | 12.4 Hz, 1H), 4.48 (d, J = 12.4 Hz, 1H), 4.00-3.95 (m, 5H), |
| | | | 3.92 (s, 3H), 3.74 (td, J = 8.7, 3.1 Hz, 2H) |
| J589 | | ESIMS m/z 654 | ¹H NMR (400 MHz, CDCl₃) δ 8.38 (t, J = 8.5 Hz, 1H), 7.92 (dd, |
| | | ([M + H]⁺) | J = 7.5, 2.4 Hz, 3H), 7.62-7.50 (m, 5H), 7.48 (s, 1H), 7.38 (d, |
| | | | J = 8.1 Hz, 1H), 7.32 (d, J = 7.8 Hz, 1H), 6.90 (s, 1H), 3.98 (d, |
| | | | J = 2.3 Hz, 2H), 2.75-2.59 (m, 1H), 2.39 (s, 3H), 1.19 (dd, J = |
| | | | 9.2, 6.8 Hz, 6H); |
| | | | ¹⁹F NMR (376 MHz, CDCl₃) δ −130.95 |
| J590 | | ESIMS m/z 654 | ¹H NMR (400 MHz, CDCl₃) δ 8.39 (t, J = 8.6 Hz, 1H), 7.99- |
| | | ([M + H]⁺) | 7.83 (m, 3H), 7.69-7.44 (m, 6H), 7.32-7.27 (m, 2H), 6.92 (s, |
| | | | 1H), 3.97 (d, J = 2.4 Hz, 2H), 2.39 (s, 3H), 2.37 (d, J = 8.4 Hz, |
| | | | 2H), 1.65-1.53 (m, 2H), 0.92 (t, J = 7.4 Hz, 3H); |
| | | | ¹⁹F NMR (376 MHz, CDCl₃) δ −130.98 |
| J591 | | ESIMS m/z 670 | ¹H NMR (400 MHz, CDCl₃) δ 8.37 (td, J = 8.6, 5.1 Hz, 1H), |
| | | ([M + H]⁺) | 7.91 (d, J = 9.0 Hz, 3H), 7.61-7.51 (m, 6H), 7.42 (d, J = 26.5 |
| | | | Hz, 2H), 6.93 (s, 1H), 4.19-4.09 (m, 1H), 3.98 (d, J = 3.6 Hz, |
| | | | 2H), 3.15 (d, J = 22.4 Hz, 3H), 2.43 (s, 3H), 1.37 (dd, J = 16.6, |
| | | | 6.4 Hz, 3H); |
| | | | ¹⁹F NMR (376 MHz, CDCl₃) δ −131.04, −131.45 |
| J592 | | ESIMS m/z 656 | ¹H NMR (400 MHz, CDCl₃) δ 8.39 (t, J = 8.5 Hz, 1H), 7.97- |
| | | ([M + H]⁺) | 7.88 (m, 3H), 7.63-7.50 (m, 5H), 7.46 (s, 1H), 7.41 (d, J = 7.8 |
| | | | Hz, 1H), 7.31 (d, J = 7.9 Hz, 1H), 7.01 (s, 1H), 4.38 (d, J = 12.5 |
| | | | Hz, 1H), 4.29 (d, J = 12.6 Hz, 1H), 3.95 (d, J = 4.1 Hz, 2H), |
| | | | 3.27 (s, 3H), 2.43 (s, 3H); |
| | | | ¹⁹F NMR (376 MHz, CDCl₃) δ −131.12 |
| J593 | | ESIMS m/z 670 | ¹H NMR (400 MHz, CDCl₃) δ 8.39 (t, J = 8.5 Hz, 1H), 7.94- |
| | | ([M + H]⁺) | 7.87 (m, 3H), 7.62-7.51 (m, 5H), 7.46 (s, 1H), 7.40 (d, J = 7.7 |
| | | | Hz, 1H), 7.30 (d, J = 7.8 Hz, 1H), 7.01 (s, 1H), 4.52-4.25 (m, |
| | | | 2H), 3.94 (s, 2H), 3.41 (q, J = 7.0 Hz, 2H), 2.43 (s, 3H), 1.15 (t, |
| | | | J = 7.0 Hz, 3H); |
| | | | ¹⁹F NMR (376 MHz, CDCl₃) δ −131.09 |
| J594 | | ESIMS m/z 684 | ¹H NMR (400 MHz, CDCl₃) δ 8.36 (t, J = 8.5 Hz, 1H), 7.95- |
| | | ([M + H]⁺) | 7.87 (m, 3H), 7.60-7.51 (m, 6H), 7.37 (d, J = 5.7 Hz, 2H), |
| | | | 6.91 (s, 1H), 4.28 (q, J = 6.7 Hz, 1H), 3.98 (d, J = 1.3 Hz, 2H), |
| | | | 3.44-3.23 (m, 2H), 2.42 (s, 3H), 1.37 (dd, J = 17.6, 6.5 Hz, |
| | | | 3H), 1.20-1.09 (m, 3H); |
| | | | ¹⁹F NMR (376 MHz, CDCl₃) δ −131.60 |
| J595 | | ESIMS m/z 670 | ¹H NMR (500 MHz, CDCl₃) δ 8.40 (t, J = 8.4 Hz, 1H), 7.96- |
| | | ([M + H]⁺) | 7.89 (m, 3H), 7.63-7.48 (m, 6H), 7.25-7.24 (m, 1H), 7.00 (d, |
| | | | J = 2.2 Hz, 1H), 6.96 (d, J = 8.4 Hz, 1H), 4.01-3.87 (m, 4H), |
| | | | 2.36 (s, 3H), 1.71 (ddd, J = 13.9, 7.5, 6.5 Hz, 2H), 0.93 (t, J = |
| | | | 7.4 Hz, 3H); |
| | | | ¹⁹F NMR (471 MHz, CDCl₃) δ −131.16 |
| J596 | | ESIMS m/z 710 | ¹H NMR (500 MHz, CDCl₃) δ 8.40 (t, J = 8.4 Hz, 1H), 7.99- |
| | | ([M + H]⁺) | 7.86 (m, 3H), 7.58 (dd, J = 12.2, 1.9 Hz, 1H), 7.56-7.51 (m, |
| | | | 4H), 7.49-7.42 (m, 1H), 7.32-7.28 (m, 1H), 7.07 (d, J = 2.2 |
| | | | Hz, 1H), 6.99 (d, J = 8.4 Hz, 1H), 4.45-4.26 (m, 2H), 4.05- |
| | | | 3.88 (m, 2H), 2.40 (s, 3H); |
| | | | ¹⁹F NMR (471 MHz, CDCl₃) δ −74.15, −131.44 |
| J597 | | ESIMS m/z 724 | ¹H NMR (500 MHz, CDCl₃) δ 8.40 (t, J = 8.4 Hz, 1H), 7.99- |
| | | ([M + H]⁺) | 7.86 (m, 3H), 7.58 (dd, J = 12.2, 1.9 Hz, 1H), 7.56-7.51 (m, |
| | | | 4H), 7.49-7.42 (m, 1H), 7.32-7.28 (m, 1H), 7.07 (d, J = 2.2 |
| | | | Hz, 1H), 6.99 (d, J = 8.4 Hz, 1H), 4.45-4.26 (m, 2H), 4.05- |
| | | | 3.88 (m, 2H), 2.40 (s, 3H); |
| | | | ¹⁹F NMR (471 MHz, CDCl₃) δ −64.58, −131.36 |
| J598 | | ESIMS m/z 724 | ¹H NMR (500 MHz, CDCl₃) δ 8.38 (t, J = 8.3 Hz, 1H), 7.96- |
| | | ([M + H]⁺) | 7.88 (m, 3H), 7.65-7.50 (m, 5H), 7.45 (dd, J = 7.2, 2.1 Hz, |
| | | | 1H), 7.41 (d, J = 7.8 Hz, 1H), 7.37-7.33 (m, 1H), 7.04 (d, J = |
| | | | 1.5 Hz, 1H), 4.63 (d, J = 12.4 Hz, 1H), 4.50 (d, J = 12.5 Hz, |
| | | | 1H), 3.95 (d, J = 4.0 Hz, 2H), 3.71 (qd, J = 8.7, 5.7 Hz, 2H), |
| | | | 2.45 (s, 3H); |
| | | | ¹⁹F NMR (471 MHz, CDCl₃) δ −73.77, −131.26 |
| J599 | | ESIMS m/z 724 | ¹H NMR (500 MHz, CDCl₃) δ 8.34 (t, J = 8.5 Hz, 1H), 7.97- |
| | | ([M + H]⁺) | 7.88 (m, 3H), 7.68 (d, J = 8.1 Hz, 1H), 7.60-7.51 (m, 5H), |
| | | | 7.43 (dd, J = 8.2, 1.5 Hz, 1H), 7.33 (d, J = 2.9 Hz, 1H), 7.02 (d, |
| | | | J = 1.6 Hz, 1H), 4.48 (q, J = 6.2 Hz, 1H), 4.10-3.89 (m, 2H), |
| | | | 3.42 (s, 3H), 2.46 (s, 3H); |
| | | | ¹⁹F NMR (471 MHz, CDCl₃) δ −76.44, −131.77 |
| J600 | | ESIMS m/z 709 | ¹H NMR (500 MHz, CDCl₃) δ 8.40 (t, J = 8.4 Hz, 1H), 7.99- |
| | | ([M + H]⁺) | 7.83 (m, 3H), 7.62 (d, J = 9.0 Hz, 1H), 7.58 (dd, J = 12.3, 1.8 |
| | | | Hz, 1H), 7.56-7.52 (m, 4H), 7.50 (d, J = 2.7 Hz, 1H), 6.80 (dd, |
| | | | J = 8.8, 2.6 Hz, 1H), 6.44 (d, J = 2.6 Hz, 1H), 4.11-3.83(m, |
| | | | 2H), 3.07 (s, 6H); |
| | | | ¹⁹F NMR (471 MHz, CDCl₃) δ −59.67, −131.30 |
| J601 | | ESIMS m/z 683 | ¹H NMR (500 MHz, CDCl₃) δ 8.41 (t, J = 8.3 Hz, 1H), 7.94- |
| | | ([M − H]⁻) | 7.88 (m, 3H), 7.62-7.51 (m, 5H), 7.49 (d, J = 2.8 Hz, 1H), |
| | | | 7.32 (d, J = 8.6 Hz, 1H), 6.79 (dd, J = 8.6, 2.7 Hz, 1H), 6.47 (d, |
| | | | J = 2.7 Hz, 1H), 4.34 (d, J = 12.3 Hz, 1H), 4.20 (d, J = 12.2 Hz, |
| | | | 1H), 4.05-3.88 (m, 2H), 3.22 (s, 3H), 3.01 (s, 6H); |
| | | | ¹⁹F NMR (471 MHz, CDCl₃) δ −130.96 |
| J602 | | ESIMS m/z 683 | ¹H NMR (500 MHz, CDCl₃) δ 8.40 (t, J = 8.5 Hz, 1H), 7.98- |
| | | ([M + H]⁺) | 7.87 (m, 3H), 7.65-7.49 (m, 6H), 7.25 (d, J = 8.7 Hz, 1H), |
| | | | 6.83 (dd, J = 8.7, 2.7 Hz, 1H), 6.40 (d, J = 2.7 Hz, 1H), 3.98 (d, |
| | | | J = 2.9 Hz, 2H), 2.97 (s, 6H), 2.30 (dd, J = 8.9, 6.6 Hz, 2H), |
| | | | 1.53 (dd, J = 14.1, 6.5 Hz, 2H), 0.91 (t, J = 7.3 Hz, 3H); |
| | | | ¹⁹F NMR (471 MHz, CDCl₃) δ −130.71 |
| J603 | | ESIMS m/z 683 | ¹H NMR (500 MHz, CDCl₃) δ 8.39 (t, J = 8.5 Hz, 1H), 7.98- |
| | | ([M + H]⁺) | 7.88 (m, 3H), 7.62-7.51 (m, 6H), 7.33 (d, J = 8.8 Hz, 1H), |
| | | | 6.89 (dd, J = 8.8, 2.8 Hz, 1H), 6.36 (d, J = 2.7 Hz, 1H), 3.98 (d, |
| | | | J = 2.5 Hz, 2H), 2.96 (s, 6H), 2.58 (p, J = 6.9 Hz, 1H), 1.17 (dd, |
| | | | J = 11.0, 6.9 Hz, 6H); |
| | | | ¹⁹F NMR (471 MHz, CDCl₃) δ −130.64 |
| J604 | | ESIMS m/z 701 | ¹H NMR (500 MHz, CDCl₃) δ 8.38 (t, J = 8.4 Hz, 1H), 7.96- |
| | | ([M + H]⁺) | 7.87 (m, 3H), 7.64-7.51 (m, 5H), 7.49 (d, J = 2.8 Hz, 1H), |
| | | | 7.08 (d, J = 14.1 Hz, 1H), 6.53 (d, J = 8.3 Hz, 1H), 3.98 (d, J = |
| | | | 2.5 Hz, 2H), 2.87 (s, 6H), 2.58 (dt, J = 13.6, 7.2 Hz, 1H), 1.16 |
| | | | (t, J = 7.2 Hz, 6H); |
| | | | ¹⁹F NMR (471 MHz, CDCl₃) δ −119.37, −130.97 |
| J605 | | ESIMS m/z 665 | ¹H NMR (500 MHz, CDCl₃) δ 8.37 (t, J = 8.5 Hz, 1H), 7.96- |
| | | ([M + H]⁺) | 7.87 (m, 3H), 7.80 (dd, J = 8.2, 1.7 Hz, 1H), 7.62 (d, J = 8.2 Hz, |
| | | | 1H), 7.58 (d, J = 1.7 Hz, 1H), 7.57-7.52 (m, 4H), 7.43 (d, J = |
| | | | 1.7 Hz, 1H), 7.42 (d, J = 3.1 Hz, 1H), 4.02 (d, J = 1.7 Hz, 2H), |
| | | | 2.77 (tt, J = 13.7, 6.9 Hz, 1H), 1.30-1.14 (m, 6H); |
| | | | ¹⁹F NMR (471 MHz, CDCl₃) δ −131.19 |
| J606 | | ESIMS m/z 681 | ¹H NMR (500 MHz, CDCl₃) δ 8.35 (q, J = 8.9 Hz, 1H), 7.97- |
| | | ([M + H]⁺) | 7.88 (m, 3H), 7.86 (d, J = 8.3 Hz, 1H), 7.80 (dd, J = 8.2, 3.1 Hz, |
| | | | 1H), 7.62-7.53 (m, 5H), 7.49-7.43 (m, 2H), 4.30-4.17 (m, |
| | | | 1H), 4.09-4.00 (m, 2H), 3.18 (d, J = 12.3 Hz, 3H), 1.36 (ddd, |
| | | | J = 12.6, 6.5, 1.9 Hz, 3H); |
| | | | ¹⁹F NMR (471 MHz, CDCl₃) δ −131.13, −131.40 |
| J607 | | ESIMS m/z 614 | ¹H NMR (500 MHz, CDCl₃) δ 8.60 (t, J = 8.2 Hz, 1H), 8.21- |
| | | ([M + H]⁺) | 8.14 (m, 2H), 7.89 (d, J = 8.6 Hz, 1H), 7.84 (dd, J = 11.3, 1.9 |
| | | | Hz, 1H), 7.60 (d, J = 3.2 Hz, 1H), 7.41-7.32 (m, 4H), 6.90 (s, |
| | | | 1H), 4.01 (d, J = 3.8 Hz, 2H), 2.65 (p, J = 6.8 Hz, 1H), 2.39 (s, |
| | | | 3H), 1.19 (dd, J = 9.1, 6.8 Hz, 6H); |
| | | | ¹⁹F NMR (471 MHz, CDCl₃) δ −57.65, −129.62 |
| J608 | | ESIMS m/z 614 | ¹H NMR (500 MHz, CDCl₃) δ 8.61 (t, J = 8.2 Hz, 1H), 8.22- |
| | | ([M + H]⁺) | 8.13 (m, 2H), 7.89 (d, J = 8.5 Hz, 1H), 7.85 (dd, J = 11.3, 1.9 |
| | | | Hz, 1H), 7.62 (d, J = 3.1 Hz, 1H), 7.38 (dd, J = 8.3, 5.5 Hz, 2H), |
| | | | 7.34-7.26 (m, 2H), 6.92 (d, J = 1.6 Hz, 1H), 4.00 (d, J = 4.0 |
| | | | Hz, 2H), 2.40 (s, 3H), 2.41-2.32 (m, 2H), 1.60 (dd, J = 15.2, |
| | | | 7.6 Hz, 2H), 0.92 (t, J = 7.3 Hz, 3H); |
| | | | ¹⁹F NMR (471 MHz, CDCl₃) δ −57.65, −129.65 |
| J609 | | ESIMS m/z 616 | ¹H NMR (500 MHz, CDCl₃) δ 8.61 (t, J = 8.3 Hz, 1H), 8.21- |
| | | ([M + H]⁺) | 8.15 (m, 2H), 7.89 (d, J = 8.7 Hz, 1H), 7.87-7.82 (m, 1H), |
| | | | 7.58 (d, J = 3.2 Hz, 1H), 7.40 (dd, J = 13.2, 8.1 Hz, 3H), 7.33 |
| | | | (d, J = 7.7 Hz, 1H), 7.01 (s, 1H), 4.39 (d, J = 12.6 Hz, 1H), 4.29 |
| | | | (d, J = 12.5 Hz, 1H), 4.05-3.91 (m, 2H), 3.27 (s, 3H), 2.44 (s, |
| | | | 3H); |
| | | | ¹⁹F NMR (471 MHz, CDCl₃) δ −57.65, −129.81 |
| J610 | | ESIMS m/z 630 | ¹H NMR (500 MHz, CDCl₃) δ 8.61 (t, J = 8.2 Hz, 1H), 8.23- |
| | | ([M + H]⁺) | 8.13 (m, 2H), 7.89 (d, J = 8.6 Hz, 1H), 7.84 (dd, J = 11.3, 1.9 |
| | | | Hz, 1H), 7.59 (d, J = 3.1 Hz, 1H), 7.39 (t, J = 8.0 Hz, 3H), 7.31 |
| | | | (d, J = 8.0 Hz, 1H), 7.01 (d, J = 1.5 Hz, 1H), 4.43 (d, J = 12.5 |
| | | | Hz, 1H), 4.36 (d, J = 12.5 Hz, 1H), 3.97 (d, J = 1.1 Hz, 2H), |
| | | | 3.41 (q, J = 7.0 Hz, 2H), 2.43 (s, 3H), 1.15 (t, J = 7.0 Hz, 3H); |
| | | | ¹⁹F NMR (471 MHz, CDCl₃) δ −57.65, −129.78 |
| J611 | | ESIMS m/z 669 | ¹H NMR (500 MHz, CDCl₃) δ 8.61 (t, J = 8.2 Hz, 1H), 8.23- |
| | | ([M + H]⁺) | 8.14 (m, 2H), 7.89 (d, J = 8.8 Hz, 1H), 7.85 (dd, J = 11.4, 1.9 |
| | | | Hz, 1H), 7.63 (d, J = 9.0 Hz, 1H), 7.61 (d, J = 3.0 Hz, 1H), 7.41- |
| | | | 7.36 (m, 2H), 6.81 (dd, J = 8.9, 2.5 Hz, 1H), 6.44 (d, J = 2.6 |
| | | | Hz, 1H), 4.12-3.87 (m, 2H), 3.08 (s, 6H); |
| | | | ¹⁹F NMR (471 MHz, CDCl₃) δ −57.65, −59.65, −130.00 |
| J612 | | ESIMS m/z 643 | ¹H NMR (500 MHz, CDCl₃) δ 8.62 (t, J = 8.3 Hz, 1H), 8.23- |
| | | ([M − H]⁻) | 8.13 (m, 2H), 7.89 (d, J = 9.3 Hz, 1H), 7.86-7.82 (m, 1H), |
| | | | 7.63 (d, J = 3.1 Hz, 1H), 7.42-7.36 (m, 2H), 7.32 (d, J = 8.6 |
| | | | Hz, 1H), 6.80 (dd, J = 8.6, 2.6 Hz, 1H), 6.47 (d, J = 2.6 Hz, 1H), |
| | | | 4.34 (d, J = 12.3 Hz, 1H), 4.20 (d, J = 12.2 Hz, 1H), 4.01-3.90 |
| | | | (m, 2H), 3.22 (s, 3H), 3.01 (s, 6H); |
| | | | ¹⁹F NMR (471 MHz, CDCl₃) δ −57.65, −129.64 |
| J613 | | ESIMS m/z 650 | ¹H NMR (500 MHz, CDCl₃) δ 8.05 (d, J = 8.3 Hz, 1H), 7.98- |
| | | ([M + H]⁺) | 7.87 (m, 3H), 7.68 (d, J = 2.4 Hz, 1H), 7.62 (dd, J = 8.3, 2.1 Hz, |
| | | | 1H), 7.58-7.51 (m, 3H), 7.38 (d, J = 8.0 Hz, 1H), 7.34-7.29 |
| | | | (m, 1H), 7.06 (s, 1H), 6.92 (d, J = 1.7 Hz, 1H), 3.97 (d, J = 1.4 |
| | | | Hz, 2H), 2.68 (p, J = 6.8 Hz, 1H), 2.39 (s, 3H), 2.23 (s, 3H), |
| | | | 1.22 (d, J = 6.8 Hz, 3H), 1.18 (d, J = 6.9 Hz, 3H) |
| J614 | | ESIMS m/z 666 | ¹H NMR (500 MHz, CDCl₃) δ 8.04 (dd, J = 8.5, 3.9 Hz, 1H), |
| | | ([M + H]⁺) | 7.97-7.88 (m, 3H), 7.71-7.65 (m, 1H), 7.65-7.60 (m, 1H), |
| | | | 7.57-7.51 (m, 4H), 7.38 (t, J = 6.9 Hz, 1H), 7.00 (d, J = 27.5 |
| | | | Hz, 1H), 6.94 (d, J = 5.1 Hz, 1H), 4.16 (ddd, J = 23.6, 11.8, 5.5 |
| | | | Hz, 1H), 4.04-3.87 (m, 2H), 3.16 (d, J = 48.4 Hz, 3H), 2.42 (d, |
| | | | J = 2.5 Hz, 3H), 2.20 (d, J = 29.0 Hz, 3H), 1.38 (dd, J = 33.2, |
| | | | 6.4 Hz, 3H) |
| J615 | | ESIMS m/z 666 | ¹H NMR (500 MHz, CDCl₃) δ 8.06 (d, J = 8.5 Hz, 1H), 7.97- |
| | | ([M + H]⁺) | 7.88 (m, 3H), 7.68 (d, J = 2.7 Hz, 1H), 7.62 (dd, J = 8.4, 2.1 Hz, |
| | | | 1H), 7.54 (d, J = 7.9 Hz, 3H), 7.41 (d, J = 7.8 Hz, 1H), 7.30 (d, |
| | | | J = 7.8 Hz, 1H), 7.04 (s, 1H), 7.03 (s, 1H), 4.43 (d, J = 12.6 Hz, |
| | | | 1H), 4.37 (d, J = 12.5 Hz, 1H), 3.93 (d, J = 1.0 Hz, 2H), 3.43 (q, |
| | | | J = 7.0 Hz, 2H), 2.43 (s, 3H), 2.24 (s, 3H), 1.16 (t, J = 7.0 Hz, |
| | | | 3H) |
| J616 | | ESIMS m/z 720 | ¹H NMR (500 MHz, CDCl₃) δ 8.05 (d, J = 8.5 Hz, 1H), 7.93 (d, |
| | | ([M + H]⁺) | J = 1.4 Hz, 1H), 7.91 (d, J = 9.1 Hz, 2H), 7.68 (d, J = 2.0 Hz, |
| | | | 1H), 7.62 (dd, J = 8.5, 2.1 Hz, 1H), 7.57-7.51 (m, 3H), 7.43 |
| | | | (d, J = 7.9 Hz, 1H), 7.34 (d, J = 7.5 Hz, 1H), 7.06 (d, J = 1.6 |
| | | | Hz, 1H), 7.00 (s, 1H), 4.63 (d, J = 12.4 Hz, 1H), 4.51 (d, J = |
| | | | 12.4 Hz, 1H), 3.93 (d, J = 3.1 Hz, 2H), 3.73 (qd, J = 8.7, 1.5 |
| | | | Hz, 2H), 2.45 (s, 3H), 2.24 (s, 3H); |
| | | | ¹⁹F NMR (471 MHz, CDCl₃) δ −73.75 |
| J617 | | ESIMS m/z 720 | ¹H NMR (500 MHz, CDCl₃) δ 8.03 (d, J = 8.4 Hz, 1H), 7.93 (d, |
| | | ([M + H]⁺) | J = 1.4 Hz, 1H), 7.91 (d, J = 9.0 Hz, 2H), 7.70-7.65 (m, 2H), |
| | | | 7.63 (dd, J = 8.4, 2.1 Hz, 1H), 7.57-7.52 (m, 3H), 7.45-7.40 |
| | | | (m, 1H), 7.03 (d, J = 1.6 Hz, 1H), 6.92 (s, 1H), 4.50 (q, J = 6.2 |
| | | | Hz, 1H), 4.08-3.88 (m, 2H), 3.45 (s, 3H), 2.46 (d, J = 2.3 Hz, |
| | | | 3H), 2.17 (s, 3H); |
| | | | ¹⁹F NMR (471 MHz, CDCl₃) δ −76.44 |
| J618 | | ESIMS m/z 679 | ¹H NMR (500 MHz, CDCl₃) δ 8.05 (d, J = 8.4 Hz, 1H), 7.93 (d, |
| | | ([M + H]⁺) | J = 1.4 Hz, 1H), 7.91 (d, J = 9.0 Hz, 2H), 7.68 (d, J = 2.1 Hz, |
| | | | 1H), 7.62 (dd, J = 8.4, 2.1 Hz, 1H), 7.58-7.52 (m, 3H), 7.33 |
| | | | (d, J = 8.8 Hz, 1H), 7.11 (s, 1H), 6.88 (dd, J = 8.8, 2.8 Hz, 1H), |
| | | | 6.38 (d, J = 2.7 Hz, 1H), 3.96 (d, J = 1.0 Hz, 2H), 2.96 (s, 6H), |
| | | | 2.61 (p, J = 6.8 Hz, 1H), 2.24 (s, 3H), 1.20 (d, J = 6.8 Hz, 3H), |
| | | | 1.16 (d, J = 6.9 Hz, 3H) |
| J619 | | ESIMS m/z 697 | ¹H NMR (500 MHz, CDCl₃) δ 8.06 (d, J = 8.4 Hz, 1H), 7.94 (d, |
| | | ([M + H]⁺) | J = 1.4 Hz, 1H), 7.91 (d, J = 9.0 Hz, 2H), 7.70-7.68 (m, 1H), |
| | | | 7.63 (dd, J = 8.5, 2.1 Hz, 1H), 7.57-7.52 (m, 3H), 7.10-7.04 |
| | | | (m, 2H), 6.55 (d, J = 8.4 Hz, 1H), 3.96 (d, J = 1.3 Hz, 2H), 2.87 |
| | | | (s, 6H), 2.60 (dt, J = 12.9, 6.6 Hz, 1H), 2.25 (s, 3H), 1.17 (dd, J = |
| | | | 13.0, 6.8 Hz, 6H); |
| | | | ¹⁹F NMR (471 MHz, CDCl₃) δ −119.54 |
| J620 | | HRMS-ESI (m/z) | ¹H NMR (400 MHz, CDCl₃) δ 8.47 (t, J = 8.4 Hz, 1H), 8.43 (s, |
| | | [M + H]⁺ calcd for | 1H), 8.02-7.92 (m, 1H), 7.88 (dd, J = 12.2, 1.8 Hz, 1H), 7.65- |
| | | C₃₁H₂₈F₄N₆O₄S, | 7.55 (m, 2H), 7.49 (d, J = 3.1 Hz, 1H), 7.28 (s, 1H), 7.05- |
| | | 657.1902; found, | 6.96 (m, 3H), 4.60 (d, J = 12.3 Hz, 1H), 4.47 (d, J = 12.4 Hz, |
| | | 657.1904 | 1H), 4.09 (q, J = 7.0 Hz, 2H), 3.95 (d, J = 2.4 Hz, 2H), 3.71 (qd, |
| | | | J = 8.7, 3.8 Hz, 2H), 2.35 (d, J = 10.2 Hz, 6H), 1.45 (t, J = 6.9 |
| | | | Hz, 3H); |
| | | | ¹⁹F NMR (376 MHz, CDCl₃) δ −73.77, −131.63 |
| J621 | | ESIMS m/z 776 | ¹H NMR (500 MHz, CDCl₃) δ 8.55 (s, 1H), 8.43 (t, J = 8.4 Hz, |
| | | ([M − H]⁻) | 1H), 7.96 (dd, J = 8.6, 1.8 Hz, 1H), 7.87 (dd, J = 12.0, 1.8 Hz, |
| | | | 1H), 7.84-7.75 (m, 1H), 7.62 (ddd, J = 24.7, 8.7, 5.5 Hz, 1H), |
| | | | 7.45 (dd, J = 7.7, 3.7 Hz, 2H), 7.41-7.28 (m, 3H), 7.26-7.15 |
| | | | (m, 2H), 7.03 (s, 1H), 4.64-4.37 (m, 5H), 4.07-3.81 (m, 2H), |
| | | | 2.43 (d, J = 24.9 Hz, 3H); |
| | | | ¹³C NMR (126 MHz, CDCl₃) δ 172.18, 170.87, 162.34, 162.25, |
| | | | 160.37, 160.28, 159.25, 152.86, 150.93, 148.42, 141.59, |
| | | | 139.91, 135.43, 133.67, 132.57, 132.36, 131.97, 131.16, |
| | | | 131.10, 130.86, 130.64, 130.58, 130.21, 129.81, 129.58, |
| | | | 129.33, 129.06, 128.81, 127.74, 127.66, 126.20, 126.13, |
| | | | 124.46, 122.87, 122.43, 121.39, 121.20, 120.08, 119.34, |
| | | | 118.85, 118.68, 113.28, 113.23, 113.04, 112.87, 69.96, 67.87, |
| | | | 67.63, 53.45, 33.05, 21.03; |
| | | | ¹⁹F NMR (471 MHz, CDCl₃) δ −58.01, −60.77, −113.28, −131.28 |
| J622 | | ESIMS m/z 779 | ¹H NMR (500 MHz, CDCl₃) δ 8.55 (s, 1H), 8.47 (t, J = 8.4 Hz, |
| | | ([M − H]⁻) | 1H), 8.01-7.84(m, 1H), 7.83-7.74 (m, 1H), 7.53 (d, J = 3.0 |
| | | | Hz, 1H), 7.48-7.28 (m, 4H), 7.02 (s, 1H), 4.55-4.38 (m, 5H), |
| | | | 4.01 (d, J = 56.1 Hz, 2H), 2.39 (s, 3H); |
| | | | ¹³C NMR (126 MHz, CDCl₃) δ 172.19, 170.70, 162.26, 159.28, |
| | | | 151.02, 148.42, 141.59, 139.95, 135.43, 133.55, 131.67, |
| | | | 130.87, 130.06, 129.78, 129.48, 127.77, 127.69, 126.23, |
| | | | 122.91, 122.43, 121.39, 121.20, 120.18, 113.06, 112.89, |
| | | | 69.74, 59.15, 59.00, 33.00, 29.71, 21.12 |
| J623 | | ESIMS m/z 695 | ¹H NMR (500 MHz, CDCl₃) δ 8.54 (s, 1H), 8.48 (t, J = 8.4 Hz, |
| | | ([M − H]⁻) | 1H), 7.96 (dd, J = 8.6, 1.8 Hz, 1H), 7.88 (dd, J = 12.0, 1.9 Hz, |
| | | | 1H), 7.82-7.75 (m, 2H), 7.53 (d, J = 3.0 Hz, 1H), 7.46-7.35 |
| | | | (m, 3H), 7.35-7.27 (m, 1H), 7.24-7.16 (m, 1H), 7.07-6.98 |
| | | | (m, 2H), 4.52-4.34 (m, 4H), 3.89-3.64 (m, 2H), 2.42 (d, J = |
| | | | 23.3 Hz, 3H); |
| | | | ¹³C NMR (126 MHz, CDCl₃) δ 172.34, 170.87, 162.27, 162.25, |
| | | | 159.42, 152.93, 151.00, 148.42, 141.58, 139.56, 138.81, |
| | | | 135.42, 133.71, 132.34, 130.72, 130.17, 129.54, 127.89, |
| | | | 127.81, 127.46, 127.30, 126.13, 126.07, 126.03, 125.94, |
| | | | 123.04, 122.85, 122.42, 121.39, 121.19, 120.20, 119.33, |
| | | | 113.05, 112.88, 69.22, 67.50, 32.96 |
| J624 | | ESIMS m/z 655 | ¹H NMR (500 MHz, CDCl₃) δ 8.54 (s, 1H), 8.48 (t, J = 8.4 Hz, |
| | | ([M + H]⁺) | 1H), 7.96 (dd, J = 8.7, 1.8 Hz, 1H), 7.88 (dd, J = 12.0, 1.9 Hz, |
| | | | 1H), 7.82-7.75 (m, 2H), 7.57 (d, J = 3.0 Hz, 1H), 7.44-7.37 |
| | | | (m, 3H), 7.31 (dd, J = 7.7, 1.8 Hz, 1H), 7.02 (t, J = 1.2 Hz, 1H), |
| | | | 4.52-4.34 (m, 2H), 4.05-3.89 (m, 2H), 3.20 (d, J = 6.9 Hz, |
| | | | 2H), 2.43 (s, 3H), 0.53-0.41 (m, 2H), 0.23-0.09 (m, 2H); |
| | | | ¹³C NMR (126 MHz, CDCl₃) δ 172.27, 170.84, 162.28, 159.46, |
| | | | 152.97, 151.03, 148.44, 141.58, 139.31, 135.43, 133.51, |
| | | | 132.81, 130.69, 129.89, 129.41, 126.16, 122.86, 122.43, |
| | | | 121.20, 120.21, 119.34, 112.91, 75.28, 69.67, 33.09, 21.09, |
| | | | 10.45, 2.91 |
| J625 | | HRMS-ESI (m/z) | ¹H NMR (400 MHz, CDCl₃) δ 8.46 (t, J = 8.3 Hz, 1H), 8.43 (s, |
| | | [M + H]⁺ calcd for | 1H), 8.04-7.92 (m, 1H), 7.92-7.83 (m, 1H), 7.65-7.57 (m, |
| | | C₃₀H₂₆F₄N₆O₄S, | 2H), 7.47 (d, J = 3.0 Hz, 1H), 7.42 (d, J = 7.8 Hz, 1H), 7.34 (d, |
| | | 643.1745; found, | J = 7.7 Hz, 1H), 7.07-6.97 (m, 3H), 4.63 (d, J = 12.4 Hz, 1H), |
| | | 643.1745 | 4.50 (d, J = 12.4 Hz, 1H), 4.09 (q, J = 7.0 Hz, 2H), 3.96 (d, J = |
| | | | 2.8 Hz, 2H), 3.71 (qt, J = 8.0, 4.1 Hz, 2H), 2.45 (s, 3H), 1.45 (t, |
| | | | J = 7.0 Hz, 3H); |
| | | | ¹⁹F NMR (376 MHz, CDCl₃) δ −73.78, −131.56 |
| J626 | | ESIMS m/z 775 | ¹H NMR (400 MHz, CDCl₃) δ 8.66 (s, 1H), 8.51-8.32 (m, 3H), |
| | | ([M + H]⁺) | 7.98-7.86 (m, 4H), 7.50 (s, 1H), 7.43-7.30 (m, 2H), 7.05 (s, |
| | | | 1H), 4.70-4.44 (m, 2H), 3.97 (d, J = 2.2 Hz, 2H), 3.77-3.62 |
| | | | (m, 2H), 2.45 (s, 3H); |
| | | | ¹⁹F NMR (376 MHz, CDCl₃) δ −60.77, −73.87 |
| J627 | | ESIMS m/z 721 | ¹H NMR (400 MHz, CDCl₃) δ 8.66 (s, 1H), 8.52-8.30 (m, 3H), |
| | | ([M + H]⁺) | 8.00-7.83 (m, 4H), 7.52 (s, 1H), 7.39 (d, J = 7.8 Hz, 1H), 7.30 |
| | | | (d, J = 7.8 Hz, 1H), 7.02 (s, 1H), 4.40 (q, J = 12.7 Hz, 2H), 3.96 |
| | | | (s, 2H), 3.39 (q, J = 7.0 Hz, 2H), 2.43 (s, 3H), 1.14 (t, J = 7.0 |
| | | | Hz, 3H) |
| J628 | | ESIMS m/z 705 | ¹H NMR (400 MHz, CDCl₃) δ 8.66 (s, 1H), 8.51-8.31 (m, 3H), |
| | | ([M + H]⁺) | 7.97-7.85 (m, 4H), 7.54 (s, 1H), 7.28 (q, J = 8.0 Hz, 2H), 6.92 |
| | | | (s, 1H), 3.99 (d, J = 1.2 Hz, 2H), 2.48-2.28 (m, 5H), 1.65- |
| | | | 1.53 (m, 2H), 0.91 (t, J = 7.3 Hz, 3H) |
| J629 | | ESIMS m/z 775 | ¹H NMR (400 MHz, CDCl₃) δ 8.66 (d, J = 0.9 Hz, 1H), 8.53- |
| | | ([M + H]⁺) | 8.31 (m, 3H), 8.04-7.77 (m, 4H), 7.53 (s, 1H), 7.28 (d, J = 6.7 |
| | | | Hz, 1H), 7.03 (s, 1H), 6.96 (d, J = 8.5 Hz, 1H), 4.26-4.19 (m, |
| | | | 2H), 3.94 (s, 2H), 2.61-2.46 (m, 2H), 2.37 (s, 3H) |
| J630 | | ESIMS m/z 734 | ¹H NMR (400 MHz, CDCl₃) δ 8.66 (s, 1H), 8.50-8.31 (m, 3H), |
| | | ([M + H]⁺) | 7.97-7.85 (m, 4H), 7.58 (s, 1H), 7.32 (d, J = 8.7 Hz, 1H), 6.88 |
| | | | (dd, J = 8.9, 2.8 Hz, 1H), 6.35 (d, J = 2.7 Hz, 1H), 4.00 (s, 2H), |
| | | | 2.96 (s, 6H), 2.57 (q, J = 6.8 Hz, 1H), 1.17 (dd, J = 17.4, 6.9 |
| | | | Hz, 6H) |
| J631 | | ESIMS m/z 735 | ¹H NMR (400 MHz, CDCl₃) δ 8.66 (s, 1H), 8.53-8.30 (m, 3H), |
| | | ([M + H]⁺) | 7.97-7.85 (m, 4H), 7.60-7.45 (m, 2H), 7.37 (d, J = 8.1 Hz, |
| | | | 1H), 6.90 (s, 1H), 4.26 (dd, J = 22.2, 6.6 Hz, 1H), 4.00 (s, 2H), |
| | | | 3.45-3.13 (m, 2H), 2.42 (s, 3H), 1.34 (d, J = 6.5 Hz, 3H), 1.20- |
| | | | 1.05 (m, 3H) |
| J632 | | ESIMS m/z 789 | ¹H NMR (400 MHz, CDCl₃) δ 8.66 (s, 1H), 8.53-8.30 (m, 3H), |
| | | ([M + H]⁺) | 7.97-7.85 (m, 4H), 7.50 (s, 1H), 7.26 (s, 1H), 6.99 (s, 1H), |
| | | | 4.64-4.44 (m, 2H), 3.95 (s, 2H), 3.70 (dd, J = 8.9, 3.3 Hz, |
| | | | 2H), 2.36 (s, 3H), 2.33 (s, 3H) |
| J633 | | ESIMS m/z 821 | ¹H NMR (400 MHz, CDCl₃) δ 8.66 (s, 1H), 8.54-8.30 (m, 3H), |
| | | ([M + H]⁺) | 7.98-7.85 (m, 4H), 7.53 (s, 1H), 7.01 (s, 1H), 6.69 (s, 1H), |
| | | | 4.62-4.43 (m, 2H), 4.04-3.94 (m, 5H), 3.91 (s, 3H), 3.74 (q, |
| | | | J = 8.8 Hz, 2H) |
| J634 | | HRMS-ESI (m/z) | ¹H NMR (400 MHz, CDCl₃) δ 8.54 (d, J = 1.0 Hz, 1H), 8.48 (t, J = |
| | | [M + H]⁺ calcd for | 8.3 Hz, 1H), 7.97 (d, J = 8.6 Hz, 1H), 7.88 (d, J = 12.0 Hz, |
| | | C₃₀H₂₃F₇N₆O₄S, | 1H), 7.78 (d, J = 8.7 Hz, 2H), 7.46 (d, J = 9.1 Hz, 1H), 7.41- |
| | | 697.1462; found, | 7.32 (m, 4H), 7.05 (s, 1H), 4.63 (d, J = 12.4 Hz, 1H), 4.51 (d, J = |
| | | 697.1462 | 12.4 Hz, 1H), 3.96 (dd, J = 2.3, 1.0 Hz, 2H), 3.72 (qd, J = 8.7, |
| | | | 3.8 Hz, 2H), 2.76 (q, J = 7.6 Hz, 2H), 1.31 (t, J = 7.6 Hz, 3H) |
| | | | ¹⁹F NMR (376 MHz, CDCl₃) δ −58.02, −73.77, −131.38 |
| J635 | | ESIMS m/z 708 | ¹H NMR (500 MHz, CDCl₃) δ 8.54 (s, 1H), 8.45 (t, J = 8.4 Hz, |
| | | ([M − H]⁻) | 1H), 7.96 (dd, J = 8.6, 1.8 Hz, 1H), 7.87 (dd, J = 11.9, 1.9 Hz, |
| | | | 1H), 7.82-7.75 (m, 2H), 7.49 (d, J = 3.0 Hz, 1H), 7.45 (d, J = |
| | | | 7.8 Hz, 1H), 7.41-7.31 (m, 3H), 7.20 (tdd, J = 7.5, 5.3, 1.8 Hz, |
| | | | 1H), 7.16-6.94 (m, 3H), 4.58-4.38 (m, 4H), 4.01-3.72 (m, |
| | | | 2H), 2.41 (d, J = 23.3 Hz, 3H); |
| | | | ¹³C NMR (126 MHz, CDCl₃) δ 172.20, 170.81, 162.34, 161.57, |
| | | | 159.61, 159.35, 148.46, 141.59, 139.62, 135.46, 133.70, |
| | | | 132.35, 130.76, 130.20, 129.78, 129.75, 129.53, 129.44, |
| | | | 129.37, 127.84, 126.08, 124.97, 124.85, 124.10, 124.07, |
| | | | 122.86, 122.42, 121.21, 120.21, 115.25, 115.08, 112.88, |
| | | | 69.60, 65.87, 60.40, 32.98; |
| | | | ¹⁹F NMR (471 MHz, CDCl₃) δ −58.02, −117.68, −132.51 |
| J636 | | ESIMS m/z 709 | ¹H NMR (500 MHz, CDCl₃) δ 8.59-8.43 (m, 1H), 8.01-7.86 |
| | | ([M − H]⁻) | (m, 1H), 7.85-7.75 (m, 2H), 7.66-7.52 (m, 2H), 7.51-7.29 |
| | | | (m, 9H), 7.14 (d, J = 8.2 Hz, 1H), 4.52-4.40 (m, 5H), 3.97- |
| | | | 3.67 (m, 2H); |
| | | | ¹³C NMR (126 MHz, CDCl₃) δ 171.82, 161.79, 160.18, 159.16, |
| | | | 152.23, 147.72, 141.60, 138.92, 137.73, 137.32, 135.92, |
| | | | 135.45, 131.87, 130.59, 130.38, 129.79, 129.23, 128.49, |
| | | | 128.46, 127.92, 127.85, 127.78, 122.42, 121.21, 120.21, |
| | | | 72.77, 68.71, 34.32; |
| | | | ¹⁹F NMR (471 MHz, CDCl₃) δ −58.01, −131.07 |
| J640 | | ESIMS m/z 746 | ¹H NMR (500 MHz, CDCl₃) δ 8.54 (s, 1H), 8.35 (t, J = 8.3 Hz, |
| | | ([M − H]⁻) | 1H), 7.96-7.87 (m, 2H), 7.87-7.81 (m, 2H), 7.81-7.76 (m, |
| | | | 2H), 7.52-7.44 (m, 2H), 7.44-7.29 (m, 5H), 7.07-7.00 (m, |
| | | | 1H), 4.80 (d, J = 1.4 Hz, 2H), 4.61 (s, 2H), 4.02-3.84 (m, 2H), |
| | | | 2.45 (s, 3H); |
| | | | ¹³C NMR (126 MHz, CDCl₃) δ 172.24, 170.90, 169.11, 162.32, |
| | | | 159.27, 152.97, 151.00, 148.45, 141.58, 140.16, 135.47, |
| | | | 134.88, 133.93, 131.54, 130.87, 130.56, 129.69, 127.72, |
| | | | 126.02, 125.08, 122.95, 122.81, 122.42, 121.74, 121.42, |
| | | | 121.22, 120.16, 119.37, 113.03, 112.86, 70.07, 69.32, 59.11, |
| | | | 33.13, 21.14; |
| | | | ¹⁹F NMR (471 MHz, CDCl₃) δ −58.02, −131.03 |
| J641 | | HRMS-ESI (m/z) | ¹H NMR (400 MHz, CDCl₃) δ 8.54 (s, 1H), 8.19 (d, J = 8.5 Hz, |
| | | [M + H]⁺ calcd for | 1H), 8.04 (d, J = 8.4 Hz, 1H), 7.98 (s, 1H), 7.79 (d, J = 8.8 Hz, |
| | | C₃₁H₂₆F₆N₆O₄S, | 2H), 7.45 (d, J = 7.9 Hz, 1H), 7.37 (t, J = 7.2 Hz, 3H), 7.06 (d, J = |
| | | 693.1713; found, | 9.1 Hz, 2H), 4.63 (d, J = 12.4 Hz, 1H), 4.53 (d, J = 12.4 Hz, |
| | | 693.1714 | 1H), 3.95 (d, J = 2.1 Hz, 2H), 3.74 (q, J = 8.7 Hz, 2H), 2.75 (q, |
| | | | J = 7.6 Hz, 2H), 2.25 (s, 3H), 1.30 (t, J = 7.6 Hz, 3H); |
| | | | ¹⁹F NMR (376 MHz, CDCl₃) δ −58.03, −73.75 |
| J642 | | HRMS-ESI (m/z) | ¹H NMR (400 MHz, CDCl₃) δ 8.63 (d, J = 0.9 Hz, 1H), 8.49 (t, J = |
| | | [M + H]⁺ calcd for | 8.3 Hz, 1H), 7.98 (d, J = 8.6 Hz, 1H), 7.89 (d, J = 9.1 Hz, 3H), |
| | | C₃₀H₂₃F₇N₆O₃S, | 7.79 (d, J = 8.5 Hz, 2H), 7.54-7.47 (m, 1H), 7.47-7.32 (m, |
| | | 681.1513; found, | 2H), 7.06 (s, 1H), 4.63 (d, J = 12.4 Hz, 1H), 4.51 (d, J = 12.4 |
| | | 681.1511 | Hz, 1H), 3.96 (d, J = 2.4 Hz, 2H), 3.72 (qd, J = 8.8, 3.5 Hz, 2H), |
| | | | 2.76 (q, J = 7.6 Hz, 2H), 1.30 (t, J = 7.6 Hz, 3H); |
| | | | ¹⁹F NMR (376 MHz, CDCl₃) δ −62.50, −73.77, −131.29 |
| J643 | | HRMS-ESI (m/z) | ¹H NMR (400 MHz, CDCl₃) δ 8.51 (d, J = 7.0 Hz, 1H), 8.47 (d, |
| | | [M + H]⁺ calcd for | J = 8.4 Hz, 1H), 7.97 (d, J = 8.5 Hz, 1H), 7.88 (d, J = 11.9 Hz, |
| | | C₂₉H₂₂F₆N₆O₄S, | 1H), 7.74 (d, J = 8.7 Hz, 2H), 7.47 (s, 1H), 7.42 (d, J = 7.8 Hz, |
| | | 665.14; found, | 1H), 7.35 (d, J = 7.8 Hz, 1H), 7.29 (d, J = 8.7 Hz, 2H), 7.04 (s, |
| | | 665.1397 | 1H), 6.56 (t, J = 73.1 Hz, 1H), 4.63 (d, J = 12.3 Hz, 1H), 4.50 |
| | | | (d, J = 12.4 Hz, 1H), 3.96 (d, J = 2.7 Hz, 2H), 3.71 (qd, J = 8.7, |
| | | | 4.4 Hz, 2H), 2.45 (s, 3H); |
| | | | ¹⁹F NMR (376 MHz, CDCl₃) δ −73.78, −81.34, −131.42 |
| J644 | | HRMS-ESI (m/z) | ¹H NMR (400 MHz, CDCl₃) δ 8.52 (s, 1H), 8.48 (t, J = 8.4 Hz, |
| | | [M + H]⁺ calcd for | 1H), 7.97 (d, J = 8.6 Hz, 1H), 7.88 (d, J = 12.0 Hz, 1H), 7.74 (d, |
| | | C₃₀H₂₄F₆N₆O₄S, | J = 8.7 Hz, 2H), 7.52-7.41 (m, 2H), 7.38 (d, J = 8.0 Hz, 1H), |
| | | 679.1557; found, | 7.29 (d, J = 8.7 Hz, 2H), 7.05 (s, 1H), 6.56 (t, J = 73.1 Hz, 1H), |
| | | 679.156 | 4.63 (d, J = 12.4 Hz, 1H), 4.51 (d, J = 12.4 Hz, 1H), 3.96 (d, J = |
| | | | 2.3 Hz, 2H), 3.72 (qd, J = 8.7, 3.9 Hz, 2H), 2.76 (q, J = 7.6 Hz, |
| | | | 2H), 1.31 (t, J = 7.6 Hz, 3H); |
| | | | ¹⁹F NMR (376 MHz, CDCl₃) δ −73.77, −81.34, −131.42 |
| J645 | | ESIMS m/z 676 | ¹H NMR (500 MHz, CDCl₃) δ 8.43-8.33 (m, 1H), 8.19 (d, J = |
| | | ([M + H]⁺) | 8.6 Hz, 2H), 8.03 (d, J = 1.4 Hz, 1H), 7.82-7.69 (m, 2H), 7.62 |
| | | | (d, J = 1.3 Hz, 1H), 7.60-7.55 (m, 2H), 7.53 (dd, J = 7.9, 4.6 |
| | | | Hz, 1H), 7.42-7.35 (m, 2H), 6.93 (s, 1H), 4.15 (dq, J = 12.6, |
| | | | 6.4 Hz, 1H), 3.99 (dd, J = 3.7, 2.6 Hz, 2H), 3.15 (d, J = 28.0 |
| | | | Hz, 3H), 2.43 (s, 3H), 1.37 (dd, J = 20.3, 6.5 Hz, 3H); |
| | | | ¹⁹F NMR (471 MHz, CDCl₃) δ −78.11, −130.88, −131.28 |
| J646 | | ESIMS m/z 676 | ¹H NMR (500 MHz, CDCl₃) δ 8.41 (t, J = 8.3 Hz, 1H), 8.19 (d, J = |
| | | ([M + H]⁺) | 8.6 Hz, 2H), 8.03 (d, J = 1.4 Hz, 1H), 7.74 (d, J = 8.7 Hz, 2H), |
| | | | 7.62 (d, J = 1.4 Hz, 1H), 7.61-7.54 (m, 2H), 7.46 (d, J = 2.3 |
| | | | Hz, 1H), 7.40 (d, J = 7.8 Hz, 1H), 7.34-7.28 (m, 1H), 7.01 (d, |
| | | | J = 1.5 Hz, 1H), 4.43 (d, J = 12.6 Hz, 1H), 4.36 (d, J = 12.5 Hz, |
| | | | 1H), 3.95 (d, J = 1.3 Hz, 2H), 3.41 (q, J = 7.0 Hz, 2H), 2.43 (s, |
| | | | 3H), 1.15 (t, J = 7.0 Hz, 3H); |
| | | | ¹⁹F NMR (471 MHz, CDCl₃) δ −78.11, −130.93 |
| J647 | | ESIMS m/z 730 | ¹H NMR (500 MHz, CDCl₃) δ 8.40 (t, J = 8.4 Hz, 1H), 8.19 (d, J = |
| | | ([M + H]⁺) | 8.6 Hz, 2H), 8.03 (d, J = 1.4 Hz, 1H), 7.83-7.69 (m, 2H), |
| | | | 7.62 (d, J = 1.4 Hz, 1H), 7.61-7.54 (m, 2H), 7.45-7.39 (m, |
| | | | 2H), 7.39-7.32 (m, 1H), 7.04 (d, J = 1.6 Hz, 1H), 4.62 (d, J = |
| | | | 12.4 Hz, 1H), 4.50 (d, J = 12.4 Hz, 1H), 3.95 (d, J = 4.0 Hz, |
| | | | 2H), 3.71 (qt, J = 8.8, 4.4 Hz, 2H), 2.45 (s, 3H); |
| | | | ¹⁹F NMR (471 MHz, CDCl₃) δ −73.78, −78.11, −131.13 |
| J648 | | ESIMS m/z 712 | ¹H NMR (500 MHz, CDCl₃) δ 8.40 (t, J = 8.4 Hz, 1H), 8.19 (d, J = |
| | | ([M + H]⁺) | 8.6 Hz, 2H), 8.03 (d, J = 1.4 Hz, 1H), 7.83-7.70 (m, 2H), |
| | | | 7.62 (d, J = 1.4 Hz, 1H), 7.62-7.54 (m, 2H), 7.44 (d, J = 3.0 |
| | | | Hz, 1H), 7.40 (d, J = 7.8 Hz, 1H), 7.33 (d, J = 7.6 Hz, 1H), 7.03 |
| | | | (d, J = 1.6 Hz, 1H), 5.80 (tt, J = 55.4, 4.1 Hz, 1H), 4.56 (d, J = |
| | | | 12.4 Hz, 1H), 4.44 (d, J = 12.4 Hz, 1H), 4.10-3.87 (m, 2H), |
| | | | 3.56 (td, J = 14.0, 4.0 Hz, 2H), 2.44 (s, 3H); ¹⁹F NMR (471 MHz, |
| | | | CDCl₃) δ −78.11, −124.94 (d, J = 25.5 Hz), −131.06 |
| J649 | | ESIMS m/z 730 | ¹H NMR (500 MHz, CDCl₃) δ 8.36 (t, J = 8.5 Hz, 1H), 8.19 (d, J = |
| | | ([M + H]⁺) | 8.5 Hz, 2H), 8.03 (d, J = 1.4 Hz, 1H), 7.77-7.72 (m, 2H), |
| | | | 7.68 (d, J = 8.1 Hz, 1H), 7.63 (d, J = 1.4 Hz, 1H), 7.61-7.55 |
| | | | (m, 2H), 7.43 (d, J = 7.8 Hz, 1H), 7.33 (d, J = 3.2 Hz, 1H), 7.02 |
| | | | (s, 1H), 4.48 (q, J = 6.3 Hz, 1H), 4.07-3.90 (m, 2H), 3.43 (s, |
| | | | 3H), 2.46 (s, 3H); |
| | | | ¹⁹F NMR (471 MHz, CDCl₃) δ −76.40, −78.10, −131.65 |
| J650 | | ESIMS m/z 689 | ¹H NMR (500 MHz, CDCl₃) δ 8.41 (t, J = 8.4 Hz, 1H), 8.19 (d, J = |
| | | ([M + H]⁺) | 8.4 Hz, 2H), 8.03 (d, J = 1.3 Hz, 1H), 7.74 (d, J = 8.6 Hz, 2H), |
| | | | 7.62 (d, J = 1.4 Hz, 1H), 7.61-7.53 (m, 3H), 7.33 (d, J = 8.6 |
| | | | Hz, 1H), 6.89 (dd, J = 8.7, 2.7 Hz, 1H), 6.36 (d, J = 2.7 Hz, 1H), |
| | | | 3.98 (d, J = 2.5 Hz, 2H), 2.96 (s, 6H), 2.58 (p, J = 6.8 Hz, 1H), |
| | | | 1.17 (dd, J = 11.2, 6.8 Hz, 6H); |
| | | | ¹⁹F NMR (471 MHz, CDCl₃) δ −78.12, −130.50 |
| J651 | | ESIMS m/z 660 | ¹H NMR (500 MHz, CDCl₃) δ 8.40 (t, J = 8.4 Hz, 1H), 8.19 (d, J = |
| | | ([M + H]⁺) | 8.3 Hz, 2H), 8.03 (d, J = 1.4 Hz, 1H), 7.74 (d, J = 8.4 Hz, 2H), |
| | | | 7.62 (d, J = 1.5 Hz, 1H), 7.57 (t, J = 10.7 Hz, 2H), 7.49 (d, J = |
| | | | 2.8 Hz, 1H), 7.38 (d, J = 8.0 Hz, 1H), 7.32 (d, J = 8.0 Hz, 1H), |
| | | | 6.90 (s, 1H), 3.98 (d, J = 3.4 Hz, 2H), 2.66 (p, J = 6.9 Hz, 1H), |
| | | | 2.39 (s, 3H), 1.19 (dd, J = 11.1, 6.8 Hz, 6H); |
| | | | ¹⁹F NMR (471 MHz, CDCl₃) δ −78.12, −130.78 |
| J652 | | ESIMS m/z 687 | ¹H NMR (500 MHz, CDCl₃) δ 8.37 (q, J = 8.6 Hz, 1H), 8.19 (d, |
| | | ([M + H]⁺) | J = 8.2 Hz, 2H), 8.04 (s, 1H), 7.86 (d, J = 8.1 Hz, 1H), 7.80 (dd, |
| | | | J = 8.2, 3.2 Hz, 1H), 7.77-7.72 (m, 2H), 7.63 (s, 1H), 7.59 (t, J = |
| | | | 11.1 Hz, 2H), 7.46 (d, J = 1.7 Hz, 1H), 7.38 (d, J = 35.8 Hz, |
| | | | 1H), 4.23 (p, J = 6.7 Hz, 1H), 4.02 (d, J = 3.0 Hz, 2H), 3.18 (d, |
| | | | J = 11.8 Hz, 3H), 1.36 (dd, J = 10.9, 6.5 Hz, 3H); |
| | | | ¹⁹F NMR (471 MHz, CDCl₃) δ −78.10, −130.98, −131.26 |
| J653 | | HRMS-ESI (m/z) | ¹H NMR (400 MHz, CDCl₃) δ 8.54 (s, 1H), 8.19 (d, J = 8.4 Hz, |
| | | [M + H]⁺ calcd for | 1H), 8.07-7.93 (m, 2H), 7.79 (d, J = 8.6 Hz, 2H), 7.55 (d, J = |
| | | C₂₉H₂₂F₆N₆O₄S, | 9.4 Hz, 3H), 7.38 (d, J = 8.6 Hz, 2H), 7.03 (s, 1H), 4.73-4.49 |
| | | 665.14; found, | (m, 2H), 3.97 (d, J = 9.3 Hz, 2H), 3.76 (q, J = 8.6 Hz, 2H), 2.23 |
| | | 665.14 | (d, J = 16.8 Hz, 3H); |
| | | | ¹⁹F NMR (376 MHz, CDCl₃) δ −58.03, −73.77 |
| J659 | | HRMS-ESI (m/z) | 1H NMR (400 MHz, CDCl₃) δ 8.54 (s, 1H), 8.48 (t, J = 8.3 Hz, |
| | | [M + H]⁺ calcd for | 1H), 7.97 (d, J = 8.7 Hz, 1H), 7.88 (d, J = 11.9 Hz, 1H), 7.78 (d, |
| | | C₂₈H₁₉F₇N₆O₄S, | J = 8.8 Hz, 2H), 7.59-7.43 (m, 4H), 7.39 (d, J = 8.6 Hz, 2H), |
| | | 669.1149; found, | 7.23 (s, 1H), 4.71-4.51 (m, 2H), 4.01-3.94 (m, 2H), 3.80- |
| | | 669.1154 | 3.68 (m, 2H); |
| | | | ¹⁹F NMR (376 MHz, CDCl₃) δ −58.03, −74, −131.37 |
| J660 | | HRMS-ESI (m/z) | ¹H NMR (400 MHz, CDCl₃) δ 8.63 (d, J = 1.8 Hz, 1H), 8.50 (t, J = |
| | | [M + H]⁺ calcd for | 8.4 Hz, 1H), 7.98 (d, J = 8.7 Hz, 1H), 7.90 (d, J = 9.4 Hz, 3H), |
| | | C₃₀H₂₃F₇N₆O₃S, | 7.80 (d, J = 8.3 Hz, 2H), 7.49 (s, 1H), 7.28 (s, 1H), 6.99 (s, 1H), |
| | | 681.1513; found, | 4.60 (d, J = 12.3 Hz, 1H), 4.47 (d, J = 12.4 Hz, 1H), 3.95 (s, |
| | | 681.1516 | 2H), 3.71 (d, J = 9.1 Hz, 2H), 2.35 (d, J = 10.1 Hz, 6H); |
| | | | ¹⁹F NMR (376 MHz, CDCl₃) δ −62.51, −73.78, −131.40 |
| J661 | | HRMS-ESI (m/z) | ¹H NMR (400 MHz, CDCl₃) δ 8.63 (s, 1H), 8.49 (t, J = 8.3 Hz, |
| | | [M + H]⁺ calcd for | 1H), 7.98 (d, J = 8.6 Hz, 1H), 7.90 (d, J = 9.4 Hz, 3H), 7.80 (d, |
| | | C₂₈H₁₉F₇N₆O₃S, | J = 8.4 Hz, 2H), 7.61-7.50 (m, 4H), 7.46 (s, 1H), 4.67 (d, J = |
| | | 653.1200; found, | 12.5 Hz, 1H), 4.56 (d, J = 12.5 Hz, 1H), 3.98 (s, 2H), 3.75 (q, J = |
| | | 653.1197 | 9.3 Hz, 2H); |
| | | | ¹⁹F NMR (376 MHz, CDCl₃) δ −62.51, −73.79, −131.28 |
| J662 | | HRMS-ESI (m/z) | ¹H NMR (400 MHz, CDCl₃) δ 8.55 (s, 1H), 8.49 (t, J = 8.2 Hz, |
| | | [M + H]⁺ calcd for | 1H), 7.97 (d, J = 8.6 Hz, 1H), 7.88 (d, J = 11.9 Hz, 1H), 7.82- |
| | | C₃₀H₂₁F₉N₆O₄S, | 7.74 (m, 2H), 7.51 (s, 1H), 7.46-7.35 (m, 3H), 7.30 (d, J = 8.0 |
| | | 733.1274; found, | Hz, 1H), 7.01 (s, 1H), 4.48-4.32 (m, 2H), 3.95 (s, 2H), 3.41 |
| | | 733.1274 | (q, J = 6.9 Hz, 2H), 2.43 (s, 3H), 1.15 (t, J = 6.9 Hz, 3H) |
| J663 | | ESIMS m/z 679 | 1H NMR (400 MHz, CDCl₃) δ 8.55 (s, 1H), 8.49 (t, J = 8.3 Hz, |
| | | ([M + H]⁺) | 1H), 7.97 (d, J = 8.6 Hz, 1H), 7.88 (d, J = 12.0 Hz, 1H), 7.79 (d, |
| | | | J = 8.4 Hz, 2H), 7.50 (s, 1H), 7.40 (d, J = 7.9 Hz, 3H), 7.30 (d, |
| | | | J = 7.9 Hz, 1H), 7.01 (s, 1H), 4.48-4.29 (m, 2H), 3.95 (s, 2H), |
| | | | 3.41 (q, J = 7.1 Hz, 2H), 2.43 (s, 3H), 1.15 (t, J = 7.0 Hz, 3H); |
| | | | ¹⁹F NMR (376 MHz, CDCl₃) δ −85.90, −87.86, −131.16 |
| J664 | | ESIMS m/z 594 | ¹H NMR (500 MHz, DMSO-d₆) δ 9.64 (s, 1H), 9.62 (s, 1H), 9.33 |
| | | ([M + H]⁺) | (d, J = 2.0 Hz, 1H), 8.68 (t, J = 1.6 Hz, 1H), 8.23 (d, J = 8.5 Hz, |
| | | | 2H), 8.01 (d, J = 8.6 Hz, 2H), 7.18 (d, J = 7.9 Hz, 1H), 7.10 (d, |
| | | | J = 1.9 Hz, 1H), 7.04-6.99 (m, 1H), 4.16 (s, 2H), 3.13 (p, J = |
| | | | 6.8 Hz, 1H), 2.54 (s, 3H), 2.23 (s, 3H), 1.09 (d, J = 6.8 Hz, 6H); |
| | | | ¹⁹F NMR (471 MHz, DMSO-d₆) δ −60.86 |
| J665 | | HRMS-ESI (m/z) | ¹H NMR (300 MHz, CDCl₃) δ 8.55 (s, 1H), 8.48 (t, J = 8.4 Hz, |
| | | [M + H]⁺ calcd for | 1H), 7.97 (d, J = 8.7 Hz, 1H), 7.93-7.84 (m, 1H), 7.84-7.72 |
| | | C₃₀H₂₁F₉N₆O₄S, | (m, 2H), 7.52-7.44 (m, 1H), 7.44-7.34 (m, 4H), 7.05 (d, J = |
| | | 733.1274; found, | 7.4 Hz, 1H), 4.60 (d, J = 2.1 Hz, 2H), 3.98 (d, J = 0.9 Hz, 2H), |
| | | 733.1276 | 3.74 (qd, J = 8.8, 4.2 Hz, 2H), 2.51 (s, 3H) |
| J666 | | ESIMS m/z 731 | ¹H NMR (500 MHz, CDCl₃) δ 8.73 (s, 1H), 8.47 (t, J = 8.3 Hz, |
| | | ([M + H]⁺) | 1H), 8.21 (d, J = 8.7 Hz, 2H), 8.14-8.06 (m, 2H), 8.04-7.97 |
| | | | (m, 1H), 7.90 (dd, J = 11.8, 1.9 Hz, 1H), 7.68 (d, J = 8.1 Hz, |
| | | | 1H), 7.44 (d, J = 8.0 Hz, 1H), 7.39 (d, J = 3.1 Hz, 1H), 7.02 (d, |
| | | | J = 1.5 Hz, 1H), 4.47 (q, J = 6.3 Hz, 1H), 4.06-3.93 (m, 2H), |
| | | | 3.43 (s, 3H), 2.47 (s, 3H); |
| | | | ¹⁹F NMR (471 MHz, CDCl₃) δ −76.38, −78.13, −131.63 |
| J667 | | ESIMS m/z 661 | ¹H NMR (500 MHz, CDCl₃) δ 8.73 (s, 1H), 8.51 (t, J = 8.3 Hz, |
| | | ([M + H]⁺) | 1H), 8.21 (d, J = 8.7 Hz, 2H), 8.10 (d, J = 8.9 Hz, 2H), 7.99 (d, |
| | | | J = 8.5 Hz, 1H), 7.90 (dd, J = 11.8, 1.9 Hz, 1H), 7.54 (d, J = 3.0 |
| | | | Hz, 1H), 7.39 (d, J = 8.1 Hz, 1H), 7.35-7.29 (m, 1H), 6.90 (d, |
| | | | J = 1.7 Hz, 1H), 3.99 (d, J = 3.5 Hz, 2H), 2.66 (p, J = 6.8 Hz, |
| | | | 1H), 2.39 (s, 3H), 1.20 (dd, J = 11.2, 6.8 Hz, 6H); |
| | | | ¹⁹F NMR (471 MHz, CDCl₃) δ −78.13, −130.74 |
| J668 | | ESIMS m/z 695 | ¹H NMR (500 MHz, CDCl₃) δ 8.73 (s, 1H), 8.52 (t, J = 8.3 Hz, |
| | | ([M + H]⁺) | 1H), 8.21 (d, J = 8.8 Hz, 2H), 8.10 (d, J = 8.9 Hz, 2H), 7.99 (d, |
| | | | J = 8.6 Hz, 1H), 7.90 (dd, J = 11.9, 1.9 Hz, 1H), 7.51 (d, J = 3.0 |
| | | | Hz, 1H), 7.41 (d, J = 7.8 Hz, 1H), 7.32 (d, J = 7.8 Hz, 1H), 7.03 |
| | | | (d, J = 1.6 Hz, 1H), 4.58-4.51 (m, 2H), 4.48-4.40 (m, 2H), |
| | | | 4.02 (d, J = 17.9 Hz, 1H), 3.92 (d, J = 18.0 Hz, 1H), 3.68-3.59 |
| | | | (m, 1H), 3.57 (tq, J = 8.1, 3.8 Hz, 1H), 2.44 (s, 3H); |
| | | | ¹⁹F NMR (471 MHz, CDCl₃) δ −78.13, −131.00 |
| J669 | | ESIMS m/z 747 | ¹H NMR (500 MHz, CDCl₃) δ 8.73 (s, 1H), 8.51 (t, J = 8.3 Hz, |
| | | ([M + H]⁺) | 1H), 8.21 (d, J = 8.7 Hz, 2H), 8.10 (d, J = 8.9 Hz, 2H), 8.06- |
| | | | 7.97 (m, 1H), 7.90 (dd, J = 11.9, 1.8 Hz, 1H), 7.50 (d, J = 3.1 |
| | | | Hz, 1H), 7.43 (d, J = 8.5 Hz, 1H), 7.06 (dd, J = 8.5, 2.6 Hz, 1H), |
| | | | 6.77 (d, J = 2.6 Hz, 1H), 4.59 (d, J = 12.2 Hz, 1H), 4.47 (d, J = |
| | | | 12.3 Hz, 1H), 3.97 (d, J = 4.6 Hz, 2H), 3.87 (s, 3H), 3.76-3.65 |
| | | | (m, 2H); |
| | | | ¹⁹F NMR (471 MHz, CDCl₃) δ −73.73, −78.13, −131.05 |
| J670 | | ESIMS m/z 735 | ¹H NMR (500 MHz, CDCl₃) δ 8.73 (s, 1H), 8.50 (t, J = 8.3 Hz, |
| | | ([M + H]⁺) | 1H), 8.21 (d, J = 8.7 Hz, 2H), 8.10 (d, J = 8.8 Hz, 2H), 8.00 (d, |
| | | | J = 9.0 Hz, 1H), 7.91 (dd, J = 11.8, 1.8 Hz, 1H), 7.53 (dd, J = |
| | | | 8.6, 5.8 Hz, 1H), 7.49 (d, J = 3.1 Hz, 1H), 7.27 (td, J = 8.3, 2.5 |
| | | | Hz, 1H), 7.00 (dd, J = 8.3, 2.6 Hz, 1H), 4.61 (d, J = 12.4 Hz, |
| | | | 1H), 4.52 (d, J = 12.5 Hz, 1H), 3.98 (d, J = 3.0 Hz, 2H), 3.74 |
| | | | (qt, J = 8.1, 4.1 Hz, 2H); |
| | | | ¹⁹F NMR (471 MHz, CDCl₃) δ −73.77, −78.13, −110.39, −131.06 |
| J671 | | ESIMS m/z 692 | ¹H NMR (500 MHz, CDCl₃) δ 8.73 (s, 1H), 8.53 (t, J = 8.3 Hz, |
| | | ([M + H]⁺) | 1H), 8.21 (d, J = 8.7 Hz, 2H), 8.10 (d, J = 8.9 Hz, 2H), 7.98 (d, |
| | | | J = 8.6 Hz, 1H), 7.90 (dd, J = 11.9, 1.8 Hz, 1H), 7.57 (d, J = 3.0 |
| | | | Hz, 1H), 7.32 (d, J = 8.6 Hz, 1H), 6.80 (dd, J = 8.6, 2.7 Hz, 1H), |
| | | | 6.47 (d, J = 2.6 Hz, 1H), 4.34 (d, J = 12.3 Hz, 1H), 4.20 (d, J = |
| | | | 12.2 Hz, 1H), 4.02-3.89 (m, 2H), 3.22 (s, 3H), 3.01 (s, 6H); |
| | | | ¹⁹F NMR (471 MHz, CDCl₃) δ −78.14, −130.75 |
| J672 | | ESIMS m/z 746 | ¹H NMR (500 MHz, CDCl₃) δ 8.73 (s, 1H), 8.53 (t, J = 8.4 Hz, |
| | | ([M + H]⁺) | 1H), 8.21 (d, J = 8.8 Hz, 2H), 8.10 (d, J = 8.8 Hz, 2H), 7.99 (d, |
| | | | J = 8.7 Hz, 1H), 7.90 (dd, J = 11.9, 1.9 Hz, 1H), 7.55 (d, J = 3.0 |
| | | | Hz, 1H), 7.04 (d, J = 9.1 Hz, 1H), 6.82 (dd, J = 9.2, 3.1 Hz, 1H), |
| | | | 6.54 (d, J = 3.1 Hz, 1H), 4.37-4.19 (m, 2H), 4.04-3.87 (m, |
| | | | 2H), 2.97 (s, 6H); |
| | | | ¹⁹F NMR (471 MHz, CDCl₃) δ −74.38, −78.14, −131.07 |
| J673 | | ESIMS m/z 663 | ¹H NMR (400 MHz, CDCl₃) δ 8.64 (s, 1H), 8.49 (t, J = 8.3 Hz, |
| | | ([M + H]⁺) | 1H), 7.99 (d, J = 8.7 Hz, 1H), 7.96-7.87 (m, 3H), 7.80 (d, J = |
| | | | 8.4 Hz, 2H), 7.49 (d, J = 2.9 Hz, 1H), 7.32-7.22 (m, 2H), 7.07 |
| | | | (dd, J = 8.3, 5.4 Hz, 2H), 6.97-6.86 (m, 3H), 3.88-3.75 (m, |
| | | | 3H), 3.59 (d, J = 18.1 Hz, 1H), 2.41 (s, 3H); |
| | | | ¹⁹F NMR (376 MHz, CDCl₃) δ −62.50, −116.24, −131.11 |
| J674 | | ESIMS m/z 679 | ¹H NMR (400 MHz, CDCl₃) δ 8.55 (s, 1H), 8.48 (t, J = 8.4 Hz, |
| | | ([M + H]⁺) | 1H), 7.98 (d, J = 8.6 Hz, 1H), 7.94-7.87 (m, 1H), 7.83-7.76 |
| | | | (m, 2H), 7.48 (d, J = 2.5 Hz, 1H), 7.39 (d, J = 8.6 Hz, 2H), 7.32- |
| | | | 7.22 (m, 2H), 7.07 (dd, J = 8.3, 5.4 Hz, 2H), 6.97-6.88 (m, |
| | | | 3H), 3.79 (d, J = 17.8 Hz, 3H), 3.58 (d, J = 18.1 Hz, 1H), 2.41 |
| | | | (s, 3H); |
| | | | ¹⁹F NMR (376 MHz, CDCl₃) δ −58.02, −116.25, −131.20 |
| J675 | | ESIMS m/z 663 | ¹H NMR (400 MHz, CDCl₃) δ 8.64 (s, 1H), 8.48 (t, J = 8.4 Hz, |
| | | ([M + H]⁺) | 1H), 7.99 (d, J = 8.6 Hz, 1H), 7.96-7.87 (m, 3H), 7.80 (d, J = |
| | | | 8.5 Hz, 2H), 7.48 (s, 1H), 7.29 (d, J = 3.2 Hz, 2H), 7.20 (q, J = |
| | | | 7.4 Hz, 1H), 6.96 (s, 1H), 6.85 (dt, J = 26.3, 8.8 Hz, 3H), 3.81 |
| | | | (d, J = 22.5 Hz, 3H), 3.62 (d, J = 18.1 Hz, 1H), 2.42 (s, 3H); |
| | | | ¹⁹F NMR (376 MHz, CDCl₃) δ −62.50, −112.96, −131.07 |
| J676 | | ESIMS m/z 679 | ¹H NMR (400 MHz, CDCl₃) δ 8.55 (s, 1H), 8.47 (t, J = 8.3 Hz, |
| | | ([M + H]⁺) | 1H), 7.97 (d, J = 8.6 Hz, 1H), 7.90 (dd, J = 12.0, 1.8 Hz, 1H), |
| | | | 7.82-7.76 (m, 2H), 7.47 (d, J = 2.9 Hz, 1H), 7.39 (d, J = 8.6 |
| | | | Hz, 2H), 7.34-7.26 (m, 2H), 7.20 (q, J = 7.4 Hz, 1H), 6.96 (s, |
| | | | 1H), 6.85 (dt, J = 26.5, 8.8 Hz, 3H), 3.87-3.76 (m, 3H), 3.62 |
| | | | (d, J = 18.1 Hz, 1H), 2.42 (s, 3H); |
| | | | ¹⁹F NMR (376 MHz, CDCl₃) δ −58.02, −112.97, −131.16 |
| J677 | | ESIMS m/z 667 | ¹H NMR (400 MHz, CDCl₃) δ 8.63 (s, 1H), 8.49 (t, J = 8.4 Hz, |
| | | ([M + H]⁺) | 1H), 7.98 (d, J = 8.5 Hz, 1H), 7.90 (d, J = 9.4 Hz, 3H), 7.80 (d, |
| | | | J = 8.6 Hz, 2H), 7.43 (td, J = 15.0, 7.5 Hz, 3H), 7.05 (d, J = 7.5 |
| | | | Hz, 1H), 4.60 (d, J = 3.1 Hz, 2H), 3.98 (d, J = 1.5 Hz, 2H), 3.84- |
| | | | 3.62 (m, 2H), 2.51 (s, 3H); |
| | | | ¹⁹F NMR (376 MHz, CDCl₃) δ −62.47, −73.64, −131.28 |
| J678 | | ESIMS m/z 697 | ¹H NMR (400 MHz, CDCl₃) δ 8.54 (s, 1H), 8.49 (t, J = 8.4 Hz, |
| | | ([M + H]⁺) | 1H), 8.02-7.93 (m, 1H), 7.88 (dd, J = 12.0, 1.9 Hz, 1H), 7.82- |
| | | | 7.71 (m, 2H), 7.47 (d, J = 3.0 Hz, 1H), 7.43-7.36 (m, 2H), |
| | | | 7.33 (d, J = 7.7 Hz, 1H), 7.23 (d, J = 7.7 Hz, 1H), 4.61 (d, J = |
| | | | 12.5 Hz, 1H), 4.43 (d, J = 12.5 Hz, 1H), 3.98 (d, J = 2.6 Hz, |
| | | | 2H), 3.76-3.59 (m, 2H), 2.40 (s, 3H), 2.07 (s, 3H); |
| | | | ¹⁹F NMR (376 MHz, CDCl₃) δ −58.02, −73.73, −131.35 |
| J679 | | ESIMS m/z 693 | ¹H NMR (400 MHz, CDCl₃) δ 8.54 (s, 1H), 8.19 (d, J = 8.5 Hz, |
| | | ([M + H]⁺) | 1H), 8.06-7.92 (m, 2H), 7.84-7.72 (m, 2H), 7.38 (d, J = 8.5 |
| | | | Hz, 2H), 7.32 (d, J = 7.8 Hz, 1H), 7.24 (d, J = 7.8 Hz, 1H), 7.03 |
| | | | (s, 1H), 4.62 (d, J = 12.5 Hz, 1H), 4.44 (d, J = 12.5 Hz, 1H), |
| | | | 3.96 (d, J = 1.6 Hz, 2H), 3.69 (qd, J = 8.7, 4.3 Hz, 2H), 2.39 (s, |
| | | | 3H), 2.25 (s, 3H), 2.08 (s, 3H); |
| | | | ¹⁹F NMR (376 MHz, CDCl₃) δ −58.03, −73.71 |
| J680 | | ESIMS m/z 681 | ¹H NMR (400 MHz, CDCl₃) δ 8.63 (s, 1H), 8.50 (t, J = 8.4 Hz, |
| | | ([M + H]⁺) | 1H), 8.02-7.96 (m, 1H), 7.93-7.86 (m, 3H), 7.80 (d, J = 8.6 |
| | | | Hz, 2H), 7.48 (d, J = 3.1 Hz, 1H), 7.33 (d, J = 7.7 Hz, 1H), 7.23 |
| | | | (d, J = 7.8 Hz, 1H), 4.61 (d, J = 12.4 Hz, 1H), 4.43 (d, J = 12.5 |
| | | | Hz, 1H), 3.98 (d, J = 2.6 Hz, 2H), 3.75-3.58 (m, 2H), 2.40 (s, |
| | | | 3H), 2.07 (s, 3H); |
| | | | ¹⁹F NMR (376 MHz, CDCl₃) δ −62.51, −73.73, −131.29 |
| J681 | | ESIMS m/z 727 | ¹H NMR (400 MHz, CDCl₃) δ 8.54 (s, 1H), 8.18 (d, J = 8.5 Hz, |
| | | ([M + H]⁺) | 1H), 8.04 (d, J = 8.6 Hz, 1H), 7.99 (s, 1H), 7.85-7.75 (m, 2H), |
| | | | 7.47-7.32 (m, 4H), 7.05 (d, J = 5.9 Hz, 2H), 4.63 (d, J = 12.4 |
| | | | Hz, 1H), 4.51 (d, J = 12.4 Hz, 1H), 3.94 (d, J = 2.1 Hz, 2H), |
| | | | 3.73 (q, J = 8.7 Hz, 2H), 2.45 (s, 3H), 2.26 (s, 3H) |
| J682 | | ESIMS m/z 727 | ¹H NMR (400 MHz, CDCl₃) δ 8.54 (s, 1H), 8.17 (d, J = 8.5 Hz, |
| | | ([M + H]⁺) | 1H), 8.08-8.01 (m, 1H), 7.98 (s, 1H), 7.86-7.74 (m, 2H), |
| | | | 7.41 (dd, J = 16.9, 8.1 Hz, 4H), 7.10-6.99 (m, 2H), 4.67- |
| | | | 4.52 (m, 2H), 3.95 (d, J = 1.6 Hz, 2H), 3.74 (qd, J = 8.7, 3.6 Hz, |
| | | | 2H), 2.51 (s, 3H), 2.25 (s, 3H); |
| | | | ¹⁹F NMR (376 MHz, CDCl₃) δ −73.63, −85.89, −87.84 |
| J683 | | ESIMS m/z 728 | ¹H NMR (400 MHz, CDCl₃) δ 8.66 (s, 1H), 8.61 (d, J = 8.9 Hz, |
| | | ([M + H]⁺) | 1H), 8.44-8.36 (m, 2H), 7.98-7.91 (m, 2H), 7.88 (d, J = 8.8 |
| | | | Hz, 2H), 7.69 (s, 1H), 7.33-7.22 (m, 2H), 7.07 (t, J = 6.9 Hz, |
| | | | 2H), 6.99-6.90 (m, 3H), 3.86-3.77 (m, 3H), 3.64 (s, 1H), |
| | | | 2.42 (s, 3H); |
| | | | ¹⁹F NMR (376 MHz, CDCl₃) δ −116.34 |
| J684 | | HRMS-ESI (m/z) | ¹H NMR (400 MHz, CDCl₃) δ 8.60 (s, 1H), 8.19 (d, J = 8.4 Hz, |
| | | [M + H]⁺ calcd for | 1H), 8.08-7.95 (m, 2H), 7.87 (d, J = 8.2 Hz, 2H), 7.67 (d, J = |
| | | C₃₀H₂₅F₅N₆O₃S, | 8.2 Hz, 2H), 7.46-7.30 (m, 2H), 7.08-7.01 (m, 2H), 6.71 (t, J = |
| | | 645.1702; found, | 56.3 Hz, 1H), 4.66-4.46 (m, 2H), 3.94 (d, J = 2.1 Hz, 2H), |
| | | 645.1705 | 3.73 (q, J = 8.7 Hz, 2H), 2.45 (s, 3H), 2.26 (d, J = 4.2 Hz, 3H); |
| | | | ¹⁹F NMR (376 MHz, CDCl₃) δ −73.75, −110.98 |
| J685 | | ESIMS m/z 629 | ¹H NMR (500 MHz, CDCl₃) δ 8.56-8.50 (m, 2H), 8.21 (d, J = |
| | | ([M + H]⁺) | 8.5 Hz, 2H), 7.60-7.49 (m, 2H), 7.48 (d, J = 10.2 Hz, 1H), |
| | | | 7.39 (q, J = 5.1 Hz, 2H), 7.31 (d, J = 8.3 Hz, 2H), 6.93 (s, 1H), |
| | | | 4.20-4.08 (m, 1H), 4.00 (dd, J = 4.5, 2.7 Hz, 2H), 3.20-3.10 |
| | | | (m, 3H), 2.43 (d, J = 3.3 Hz, 3H), 1.44-1.31 (m, 3H); |
| | | | ¹⁹F NMR (471 MHz, CDCl₃) δ −57.71, −127.67, −128.08 |
| J686 | | HRMS-ESI (m/z) | ¹H NMR (400 MHz, CDCl₃) δ 8.61 (s, 1H), 8.48 (t, J = 8.4 Hz, |
| | | [M + H]⁺ calcd for | 1H), 8.03-7.94 (m, 1H), 7.93-7.81 (m, 3H), 7.68 (d, J = 8.3 |
| | | C₂₉H₂₂F₆N₆O₃S, | Hz, 2H), 7.48 (d, J = 3.0 Hz, 1H), 7.45-7.31 (m, 2H), 7.04 (s, |
| | | 649.1451; found, | 1H), 6.72 (t, J = 56.3 Hz, 1H), 4.68-4.40 (m, 2H), 3.96 (d, J = |
| | | 649.1442 | 2.8 Hz, 2H), 3.72 (qd, J = 8.7, 4.2 Hz, 2H), 2.45 (s, 3H); |
| | | | ¹⁹F NMR (376 MHz, CDCl₃) δ −73.78, −111.05, −131.37 |
| J687 | | ESIMS m/z 683 | ¹H NMR (500 MHz, CDCl₃) δ 8.53 (s, 1H), 8.50 (t, J = 8.7 Hz, |
| | | ([M + H]⁺) | 1H), 8.27-8.17 (m, 2H), 7.68 (d, J = 8.0 Hz, 1H), 7.56 (dd, J = |
| | | | 11.5, 2.4 Hz, 1H), 7.52-7.45 (m, 1H), 7.44 (d, J = 7.8 Hz, 1H), |
| | | | 7.35-7.29 (m, 3H), 7.02 (s, 1H), 4.47 (q, J = 6.4 Hz, 1H), 4.13- |
| | | | 3.85 (m, 2H), 3.42 (d, J = 6.7 Hz, 3H), 2.46 (d, J = 2.7 Hz, |
| | | | 3H); |
| | | | ¹⁹F NMR (471 MHz, CDCl₃) δ −57.71, −76.33, −128.46 |
| J688 | | ESIMS m/z 698 | ¹H NMR (500 MHz, CDCl₃) δ 8.56 (t, J = 8.6 Hz, 1H), 8.52 (s, |
| | | ([M + H]⁺) | 1H), 8.23-8.19 (m, 2H), 7.56 (dd, J = 11.4, 2.5 Hz, 1H), 7.53- |
| | | | 7.43 (m, 2H), 7.38-7.28 (m, 2H), 7.04 (d, J = 9.1 Hz, 1H), |
| | | | 6.82 (dd, J = 9.1, 3.0 Hz, 1H), 6.54 (d, J = 3.2 Hz, 1H), 4.41- |
| | | | 4.20 (m, 2H), 4.07-3.89 (m, 2H), 2.97 (d, J = 2.9 Hz, 6H); |
| | | | ¹⁹F NMR (471 MHz, CDCl₃) δ −57.71, −74.38, −127.93 |
| J689 | | ESIMS m/z 683 | ¹H NMR (500 MHz, CDCl₃) δ 8.55 (d, J = 8.7 Hz, 1H), 8.52 (s, |
| | | ([M + H]⁺) | 1H), 8.24-8.18 (m, 2H), 7.56 (dd, J = 11.5, 2.4 Hz, 1H), 7.49- |
| | | | 7.46 (m, 1H), 7.44 (d, J = 2.9 Hz, 1H), 7.42 (d, J = 7.8 Hz, |
| | | | 1H), 7.37-7.33 (m, 1H), 7.33-7.29 (m, 2H), 7.04 (d, J = 1.5 |
| | | | Hz, 1H), 4.62 (d, J = 12.4 Hz, 1H), 4.51 (d, J = 12.4 Hz, 1H), |
| | | | 3.96 (dd, J = 4.0, 2.3 Hz, 2H), 3.72 (qd, J = 8.7, 4.5 Hz, 2H), |
| | | | 2.45 (d, J = 3.0 Hz, 3H); |
| | | | ¹⁹F NMR (471 MHz, CDCl₃) δ −57.71, −73.79, −127.95 |
| J690 | | ESIMS m/z 687 | ¹H NMR (500 MHz, CDCl₃) δ 8.57-8.50 (m, 2H), 8.24-8.18 |
| | | ([M + H]⁺) | (m, 2H), 7.58 (dd, J = 11.4, 2.4 Hz, 1H), 7.53 (dd, J = 8.7, 5.7 |
| | | | Hz, 1H), 7.50-7.46 (m, 1H), 7.43 (d, J = 3.1 Hz, 1H), 7.33- |
| | | | 7.29 (m, 2H), 7.29-7.23 (m, 1H), 7.00 (ddd, J = 8.5, 4.2, 2.5 |
| | | | Hz, 1H), 4.64-4.57 (m, 1H), 4.53 (d, J = 12.4 Hz, 1H), 3.98 (t, |
| | | | J = 2.6 Hz, 2H), 3.74 (qt, J = 8.7, 3.7 Hz, 2H); |
| | | | ¹⁹F NMR (471 MHz, CDCl₃) δ −57.71, −73.77, −110.32, −127.90 |
| J691 | | ESIMS m/z 699 | 1H NMR (500 MHz, CDCl₃) δ 8.53 (m, 2H), 8.25-8.18 (m, 2H), |
| | | ([M + H]⁺) | 7.57 (dd, J = 11.4, 2.4 Hz, 1H), 7.50-7.46(m, 1H), 7.46- |
| | | | 7.40 (m, 2H), 7.31 (d, J = 8.3 Hz, 2H), 7.06 (dd, J = 8.5, 2.6 Hz, |
| | | | 1H), 6.78-6.73 (m, 1H), 4.58 (dd, J = 12.5, 5.2 Hz, 1H), 4.47 |
| | | | (dd, J = 12.2, 4.0 Hz, 1H), 3.97 (dd, J = 4.4, 2.3 Hz, 2H), 3.87 |
| | | | (d, J = 2.8 Hz, 3H), 3.77-3.62 (m, 2H); |
| | | | ¹⁹F NMR (471 MHz, CDCl₃) δ −57.71, −73.74, −127.90 |
| J692 | | ESIMS m/z 665 | ¹H NMR (500 MHz, CDCl₃) δ 8.57-8.51 (m, 2H), 8.24-8.18 |
| | | ([M + H]⁺) | (m, 2H), 7.56 (dd, J = 11.5, 2.4 Hz, 1H), 7.50-7.45 (m, 1H), |
| | | | 7.44 (d, J = 2.8 Hz, 1H), 7.40 (d, J = 7.8 Hz, 1H), 7.36-7.32 |
| | | | (m, 1H), 7.31 (d, J = 8.4 Hz, 2H), 7.03 (d, J = 1.6 Hz, 1H), 5.80 |
| | | | (tt, J = 55.4, 4.0 Hz, 1H), 4.56 (d, J = 12.4 Hz, 1H), 4.44 (d, J = |
| | | | 12.4 Hz, 1H), 4.09-3.83 (m, 2H), 3.57 (td, J = 14.0, 4.0 Hz, |
| | | | 2H), 2.44 (d, J = 3.0 Hz, 3H); ¹⁹F NMR (471 MHz, |
| | | | CDCl₃) δ −57.71, −124.95 (d, J = 25.5 Hz), −127.87 |
| J693 | | ESIMS m/z 683 | ¹H NMR (500 MHz, CDCl₃) δ 8.56 (t, J = 8.7 Hz, 1H), 8.52 (s, |
| | | ([M + H]⁺) | 1H), 8.26-8.18 (m, 2H), 7.56 (dd, J = 11.4, 2.4 Hz, 1H), 7.47 |
| | | | (d, J = 8.6 Hz, 2H), 7.30 (t, J = 9.0 Hz, 3H), 7.03 (d, J = 2.2 Hz, |
| | | | 1H), 6.97 (dd, J = 8.4, 3.1 Hz, 1H), 4.22 (tt, J = 9.8, 5.0 Hz, |
| | | | 2H), 3.94 (d, J = 2.4 Hz, 2H), 2.62-2.46 (m, 2H), 2.38 (s, 3H); |
| | | | ¹⁹F NMR (471 MHz, CDCl₃) δ −57.71, −64.60, −128.07 |
| J694 | | ESIMS m/z 597 | ¹H NMR (500 MHz, CDCl₃) δ 8.57-8.53 (m, 2H), 8.30 (d, J = |
| | | ([M + H]⁺) | 8.0 Hz, 2H), 7.73 (d, J = 8.1 Hz, 2H), 7.57 (dd, J = 11.4, 2.4 |
| | | | Hz, 1H), 7.49 (d, J = 10.6 Hz, 2H), 7.39 (d, J = 8.0 Hz, 1H), |
| | | | 7.36-7.31 (m, 1H), 6.90 (s, 1H), 4.00 (d, J = 3.8 Hz, 2H), 2.66 |
| | | | (p, J = 6.8 Hz, 1H), 2.39 (s, 3H), 1.20 (dd, J = 11.1, 6.9 Hz, |
| | | | 6H); |
| | | | ¹⁹F NMR (471 MHz, CDCl₃) δ −62.71, −127.51 |
| J695 | | ESIMS m/z 613 | ¹H NMR (500 MHz, CDCl₃) δ 8.58-8.50 (m, 2H), 8.30 (d, J = |
| | | ([M + H]⁺) | 8.1 Hz, 2H), 7.73 (d, J = 8.2 Hz, 2H), 7.58 (ddd, J = 11.4, 4.0, |
| | | | 2.4 Hz, 1H), 7.53 (dd, J = 8.0, 6.6 Hz, 1H), 7.51-7.45 (m, 1H), |
| | | | 7.42-7.36 (m, 2H), 6.93 (s, 1H), 4.20-4.11 (m, 1H), 4.00 |
| | | | (dd, J = 4.1, 3.2 Hz, 2H), 3.16 (d, J = 27.9 Hz, 3H), 2.43 (s, |
| | | | 3H), 1.37 (dd, J = 18.7, 6.4 Hz, 3H); |
| | | | ¹⁹F NMR (471 MHz, CDCl₃) δ −62.71, −127.59, −128.01 |
| J696 | | ESIMS m/z 667 | ¹H NMR (500 MHz, CDCl₃) δ 8.56 (s, 1H), 8.52 (t, J = 8.6 Hz, |
| | | ([M + H]⁺) | 1H), 8.30 (d, J = 8.1 Hz, 2H), 7.73 (d, J = 8.2 Hz, 2H), 7.68 (d, |
| | | | J = 8.0 Hz, 1H), 7.58 (dd, J = 11.4, 2.4 Hz, 1H), 7.50 (dd, J = |
| | | | 9.0, 2.3 Hz, 1H), 7.44 (dd, J = 8.1, 1.8 Hz, 1H), 7.34 (d, J = 2.9 |
| | | | Hz, 1H), 7.05-6.98 (m, 1H), 4.47 (q, J = 6.4 Hz, 1H), 4.12- |
| | | | 3.92 (m, 2H), 3.47-3.31 (m, 3H), 2.46 (d, J = 2.9 Hz, 3H); |
| | | | ¹⁹F NMR (471 MHz, CDCl₃) δ −62.72, −76.32, −128.39 |
| J697 | | ESIMS m/z 682 | ¹H NMR (500 MHz, CDCl₃) δ 8.61-8.54 (m, 2H), 8.30 (d, J = |
| | | ([M + H]⁺) | 8.0 Hz, 2H), 7.73 (d, J = 8.1 Hz, 2H), 7.58 (dd, J = 11.4, 2.4 |
| | | | Hz, 1H), 7.48 (dd, J = 12.6, 4.4 Hz, 2H), 7.04 (d, J = 9.1 Hz, |
| | | | 1H), 6.82 (dd, J = 9.1, 3.1 Hz, 1H), 6.54 (d, J = 3.1 Hz, 1H), |
| | | | 4.37-4.20 (m, 2H), 3.99-3.91 (m, 2H), 2.97 (s, 6H); |
| | | | ¹⁹F NMR (471 MHz, CDCl₃) δ −62.70, −74.38, −127.85 |
| J698 | | ESIMS m/z 667 | ¹H NMR (500 MHz, CDCl₃) δ 8.58-8.52 (m, 2H), 8.33-8.27 |
| | | ([M + H]⁺) | (m, 2H), 7.73 (d, J = 8.2 Hz, 2H), 7.58 (dd, J = 11.5, 2.4 Hz, |
| | | | 1H), 7.48 (dd, J = 8.9, 2.3 Hz, 1H), 7.44 (d, J = 2.8 Hz, 1H), |
| | | | 7.42 (d, J = 7.8 Hz, 1H), 7.37-7.33 (m, 1H), 7.04 (d, J = 1.5 |
| | | | Hz, 1H), 4.62 (d, J = 12.4 Hz, 1H), 4.51 (d, J = 12.4 Hz, 1H), |
| | | | 4.03-3.90 (m, 2H), 3.72 (qd, J = 8.7, 4.4 Hz, 2H), 2.45 (s, |
| | | | 3H); |
| | | | ¹⁹F NMR (471 MHz, CDCl₃) δ −62.71, −73.79, −127.87 |
| J699 | | ESIMS m/z 671 | ¹H NMR (500 MHz, CDCl₃) δ 8.58-8.51 (m, 2H), 8.30 (d, J = |
| | | ([M + H]⁺) | 8.1 Hz, 2H), 7.73 (d, J = 8.2 Hz, 2H), 7.59 (dd, J = 11.4, 2.4 |
| | | | Hz, 1H), 7.56-7.46 (m, 2H), 7.44 (s, 1H), 7.31-7.23 (m, 1H), |
| | | | 7.00 (dd, J = 8.4, 2.6 Hz, 1H), 4.61 (d, J = 12.4 Hz, 1H), 4.53 |
| | | | (d, J = 12.5 Hz, 1H), 3.98 (d, J = 3.0 Hz, 2H), 3.75 (qd, J = 8.6, |
| | | | 3.7 Hz, 2H); |
| | | | ¹⁹F NMR (471 MHz, CDCl₃) δ −62.71, −73.77, −110.32, −127.82 |
| J700 | | ESIMS m/z 683 | ¹H NMR (500 MHz, CDCl₃) δ 8.60-8.52 (m, 2H), 8.30 (d, J = |
| | | ([M + H]⁺) | 8.1 Hz, 2H), 7.73 (d, J = 8.2 Hz, 2H), 7.58 (dd, J = 11.4, 2.4 |
| | | | Hz, 1H), 7.49 (dd, J = 8.9, 2.3 Hz, 1H), 7.45 (d, J = 2.7 Hz, 1H), |
| | | | 7.43 (d, J = 8.6 Hz, 1H), 7.06 (dd, J = 8.5, 2.7 Hz, 1H), 6.76 (d, |
| | | | J = 2.5 Hz, 1H), 4.59 (d, J = 12.2 Hz, 1H), 4.48 (d, J = 12.3 Hz, |
| | | | 1H), 3.97 (d, J = 4.4 Hz, 2H), 3.87 (s, 3H), 3.70 (qt, J = 8.6, 4.3 |
| | | | Hz, 2H); ¹⁹F NMR (471 MHz, CDCl₃) |
| | | | δ −62.71, −73.74, −127.83 |
| J701 | | ESIMS m/z 649 | ¹H NMR (500 MHz, CDCl₃) δ 8.61-8.51 (m, 2H), 8.34-8.26 |
| | | ([M + H]⁺) | (m, 2H), 7.73 (d, J = 8.1 Hz, 2H), 7.58 (dd, J = 11.4, 2.4 Hz, |
| | | | 1H), 7.51-7.46 (m, 1H), 7.45 (d, J = 2.8 Hz, 1H), 7.40 (d, J = |
| | | | 7.8 Hz, 1H), 7.36-7.32 (m, 1H), 7.03 (s, 1H), 5.80 (tt, J = |
| | | | 55.4, 4.1 Hz, 1H), 4.56 (d, J = 12.4 Hz, 1H), 4.44 (d, J = 12.4 |
| | | | Hz, 1H), 4.04-3.89 (m, 2H), 3.57 (td, J = 14.0, 4.0 Hz, 2H), |
| | | | 2.45 (s, 3H); |
| | | | ¹⁹F NMR (471 MHz, CDCl₃) δ −62.71, −124.95 (d, J = 24.6 Hz), −127.80 |
| J702 | | ESIMS m/z 667 | ¹H NMR (500 MHz, CDCl₃) δ 8.64-8.53 (m, 2H), 8.30 (d, J = |
| | | ([M + H]⁺) | 8.1 Hz, 2H), 7.73 (d, J = 8.2 Hz, 2H), 7.58 (dd, J = 11.3, 2.4 |
| | | | Hz, 1H), 7.52-7.45 (m, 2H), 7.29 (dd, J = 8.5, 2.2 Hz, 1H), |
| | | | 7.03 (d, J = 2.2 Hz, 1H), 6.97 (d, J = 8.4 Hz, 1H), 4.30-4.17 |
| | | | (m, 2H), 3.94 (d, J = 2.4 Hz, 2H), 2.63-2.47 (m, 2H), 2.38 (s, |
| | | | 3H); |
| | | | ¹⁹F NMR (471 MHz, CDCl₃) δ −62.71, −64.59, −128.01 |
| J703 | | ESIMS m/z 683 | ¹H NMR (500 MHz, CDCl₃) δ 8.57-8.51 (m, 2H), 8.24-8.19 |
| | | ([M + H]⁺) | (m, 2H), 7.56 (dd, J = 11.4, 2.4 Hz, 1H), 7.45 (dt, J = 10.7, 8.4 |
| | | | Hz, 3H), 7.40 (d, J = 7.7 Hz, 1H), 7.31 (d, J = 8.4 Hz, 2H), 7.07- |
| | | | 7.02 (m, 1H), 4.67-4.53 (m, 2H), 3.98 (d, J = 2.2 Hz, 2H), |
| | | | 3.83-3.66 (m, 2H), 2.51 (d, J = 3.3 Hz, 3H); |
| | | | ¹⁹F NMR (471 MHz, CDCl₃) δ −57.71, −73.66, −127.88 |
| J704 | | ESIMS m/z 667 | ¹H NMR (500 MHz, CDCl₃) δ 8.58-8.51 (m, 2H), 8.34-8.27 |
| | | ([M + H]⁺) | (m, 2H), 7.73 (d, J = 8.2 Hz, 2H), 7.58 (dd, J = 11.4, 2.4 Hz, |
| | | | 1H), 7.49 (dd, J = 9.0, 2.0 Hz, 1H), 7.47-7.42 (m, 2H), 7.40 |
| | | | (d, J = 7.6 Hz, 1H), 7.10-7.02 (m, 1H), 4.70-4.53 (m, 2H), |
| | | | 3.98 (d, J = 2.3 Hz, 2H), 3.84-3.66 (m, 2H), 2.51 (s, 3H); |
| | | | ¹⁹F NMR (471 MHz, CDCl₃) δ −62.71, −73.66, −127.79 |
| J705 | | HRMS-ESI (m/z) | ¹H NMR (400 MHz, CDCl₃) δ 8.57 (s, 1H), 8.48 (t, J = 8.4 Hz, |
| | | [M + H]⁺ calcd for | 1H), 7.98 (dd, J = 8.6, 1.5 Hz, 1H), 7.89 (dd, J = 12.1, 1.9 Hz, |
| | | C₂₉H₂₁F₇N₆O₄S, | 1H), 7.67 (tdd, J = 3.3, 2.1, 1.0 Hz, 2H), 7.56 (t, J = 8.5 Hz, |
| | | 683.1306; found, | 1H), 7.48 (d, J = 3.1 Hz, 1H), 7.42 (d, J = 7.8 Hz, 1H), 7.39- |
| | | 683.1305 | 7.31 (m, 1H), 7.28 (dt, J = 2.3, 1.1 Hz, 1H), 7.04 (d, J = 1.7 Hz, |
| | | | 1H), 4.63 (d, J = 12.4 Hz, 1H), 4.50 (d, J = 12.4 Hz, 1H), 3.96 |
| | | | (d, J = 2.8 Hz, 2H), 3.71 (qd, J = 8.7, 4.3 Hz, 2H), 2.45 (s, 3H); |
| | | | ¹⁹F NMR (376 MHz, CDCl₃) δ −57.80, −73.78, −131.36 |
| J706 | | ESIMS m/z 683 | ¹H NMR (400 MHz, CDCl₃) δ 8.57 (s, 1H), 8.48 (t, J = 8.4 Hz, |
| | | ([M + H]⁺) | 1H), 7.97 (dd, J = 8.5, 1.4 Hz, 1H), 7.89 (dd, J = 12.0, 1.9 Hz, |
| | | | 1H), 7.67 (dd, J = 4.8, 2.2 Hz, 2H), 7.56 (t, J = 8.5 Hz, 1H), |
| | | | 7.50-7.45 (m, 1H), 7.41 (dd, J = 14.9, 7.6 Hz, 2H), 7.30- |
| | | | 7.27 (m, 1H), 7.07-7.03 (m, 1H), 4.69-4.51 (m, 2H), 3.97 (d, |
| | | | J = 1.6 Hz, 2H), 3.74 (qd, J = 8.8, 6.3 Hz, 2H), 2.51 (s, 3H); |
| | | | ¹⁹F NMR (376 MHz, CDCl₃) δ −57.80, −73.64, −131.30 |
| J707 | | ESIMS m/z 664 | ¹H NMR (400 MHz, CDCl₃) δ 9.67 (s, 2H), 9.33 (s, 1H), 8.68 (s, |
| | | ([M + H]⁺) | 1H), 8.23 (d, J = 8.4 Hz, 2H), 8.01 (d, J = 8.5 Hz, 2H), 7.32 (s, |
| | | | 1H), 7.28 (d, J = 7.8 Hz, 1H), 7.03 (d, J = 7.8 Hz, 1H), 4.62 (s, |
| | | | 2H), 4.13 (s, 2H), 4.05 (d, J = 9.3 Hz, 2H), 2.52 (s, 3H), 2.28 (s, |
| | | | 3H); |
| | | | ¹⁹F NMR (376 MHz, CDCl₃) δ −56.09, −68.01 |
| J708 | | ESIMS m/z 663 | ¹H NMR (400 MHz, CDCl₃) δ 8.64 (s, 1H), 8.46 (t, J = 8.4 Hz, |
| | | ([M + H]⁺) | 1H), 7.98 (d, J = 8.6 Hz, 1H), 7.94-7.87 (m, 3H), 7.80 (d, J = |
| | | | 8.5 Hz, 2H), 7.37-7.20 (m, 3H), 7.10 (q, J = 7.9 Hz, 2H), 7.04- |
| | | | 6.90 (m, 3H), 3.97-3.73 (m, 4H), 2.40 (s, 3H); |
| | | | ¹⁹F NMR (376 MHz, CDCl₃) δ −62.50, −117.64, −131.11 |
| J710 | | HRMS-ESI (m/z) | ¹H NMR (400 MHz, CDCl₃) δ 8.62 (s, 1H), 8.49 (t, J = 8.3 Hz, |
| | | [M + H]⁺ calcd for | 1H), 8.04 (s, 1H), 7.99 (d, J = 8.6 Hz, 1H), 7.96-7.87 (m, 2H), |
| | | C₂₉H₂₁F₇N₆O₃S, | 7.67 (d, J = 5.1 Hz, 2H), 7.48 (d, J = 3.1 Hz, 1H), 7.42 (d, J = |
| | | 667.1357; found, | 7.8 Hz, 1H), 7.35 (d, J = 8.0 Hz, 1H), 7.04 (s, 1H), 4.63 (d, J = |
| | | 667.1354 | 12.4 Hz, 1H), 4.50 (d, J = 12.4 Hz, 1H), 4.06-3.87 (m, 2H), |
| | | | 3.71 (qt, J = 7.8, 4.1 Hz, 2H), 2.45 (s, 3H); |
| | | | ¹⁹F NMR (376 MHz, CDCl₃) δ −62.81, −73.78, −131.33 |
| J711 | | HRMS-ESI (m/z) | ¹H NMR (400 MHz, CDCl₃) δ 8.57 (s, 1H), 8.49 (t, J = 8.4 Hz, |
| | | [M + H]⁺ calcd for | 1H), 7.97 (d, J = 8.7 Hz, 1H), 7.92-7.86 (m, 1H), 7.70-7.64 |
| | | C₂₉H₂₄F₄N₆O₄S, | (m, 2H), 7.56 (t, J = 8.5 Hz, 1H), 7.50 (s, 1H), 7.40 (d, J = 7.8 |
| | | 629.1589; found, | Hz, 1H), 7.30 (d, J = 7.9 Hz, 1H), 7.28 (s, 1H), 7.01 (s, 1H), |
| | | 629.16 | 4.43 (d, J = 12.6 Hz, 1H), 4.36 (d, J = 12.3 Hz, 1H), 3.95 (s, |
| | | | 2H), 3.41 (q, J = 6.9 Hz, 2H), 2.43 (s, 3H), 1.15 (t, J = 6.9 Hz, |
| | | | 3H); |
| | | | ¹⁹F NMR (376 MHz, CDCl₃) δ −57.79, −131.16 |
| J712 | | | ¹H NMR (500 MHz, CDCl₃) δ 8.54 (s, 1H), 8.46 (td, J = 8.3, 4.7 |
| | | | Hz, 1H), 7.96 (d, J = 8.8 Hz, 1H), 7.90-7.84 (m, 1H), 7.80- |
| | | | 7.75 (m, 2H), 7.51 (dd, J = 12.0, 2.6 Hz, 1H), 7.46-7.31 (m, |
| | | | 4H), 7.03 (d, J = 6.0 Hz, 1H), 6.94-6.84 (m, 1H), 4.59-4.39 |
| | | | (m, 4H), 4.05-3.74 (m, 5H), 2.45 (s, 3H); |
| | | | ¹³C NMR (126 MHz, CDCl₃) δ 174.69, 174.09, 172.26, 171.16, |
| | | | 170.85, 169.49, 162.22, 159.38, 159.25, 152.95, 151.01, |
| | | | 148.47, 145.89, 141.62, 141.49, 141.36, 140.20, 140.00, |
| | | | 135.45, 133.87, 133.61, 132.30, 131.75, 131.61, 130.84, |
| | | | 130.42, 129.65, 127.73, 126.30, 126.23, 122.88, 122.42, |
| | | | 122.29, 121.42, 121.20, 120.12, 119.37, 114.45, 114.29, |
| | | | 113.11, 112.94, 73.35, 72.91, 68.84, 67.52, 38.82, 21.12, |
| | | | 21.05; |
| | | | ¹⁹F NMR (471 MHz, CDCl₃) δ −58.00, −61.12, −131.12 |
| J714 | | ESIMS m/z 602 | ¹H NMR (500 MHz, CDCl₃) δ 8.54 (s, 1H), 8.51-8.44 (m, 1H), |
| | | ([M − H]⁻) | 7.98 (t, J = 9.1 Hz, 1H), 7.89 (dd, J = 12.0, 1.8 Hz, 1H), 7.81- |
| | | | 7.75 (m, 2H), 7.57 (dd, J = 13.5, 2.4 Hz, 1H), 7.38 (d, J = 8.7 |
| | | | Hz, 2H), 7.03-6.95 (m, 2H), 4.09 (d, J = 6.0 Hz, 1H), 3.98 (d, |
| | | | J = 1.5 Hz, 1H), 2.30 (s, 3H), 2.25 (s, 3H); |
| | | | ¹³C NMR (126 MHz, CDCl₃) δ 172.15, 171.30, 170.57, 162.63, |
| | | | 162.38, 160.65, 159.49, 153.16, 151.22, 148.59, 141.74, |
| | | | 135.58, 135.48, 131.09, 129.31, 127.92, 127.84, 126.48, |
| | | | 124.30, 124.15, 123.06, 122.55, 121.55, 121.33, 120.39, |
| | | | 119.49, 117.84, 117.65, 113.25, 113.08, 61.97, 33.11, 22.52; |
| | | | ¹⁹FNMR(471 MHz, CDCl₃) δ −57.95, −115.53, −131.38 |
| J715 | | ESIMS m/z 633 | ¹H NMR (500 MHz, CDCl₃) δ 8.53 (s, 1H), 8.48 (m, 1H), 7.96 |
| | | ([M + H]⁺) | (d, J = 8.7 Hz, 1H), 7.87 (dd, J = 12.0, 1.6 Hz, 1H), 7.79-7.75 |
| | | | (m, 2H), 7.73 (d, J = 7.9 Hz, 1H), 7.61 (t, J = 7.9 Hz, 3H), 7.58- |
| | | | 7.53 (m, 1H), 7.50-7.43 (m, 4H), 7.37 (d, J = 8.6 Hz, 4H), |
| | | | 3.99 (s, 2H); |
| | | | ¹³C NMR (126 MHz, CDCl₃) δ 172.20, 170.93, 162.25, 159.37, |
| | | | 153.04, 151.10, 148.45, 142.86, 141.62, 139.77, 135.46, |
| | | | 135.07, 129.87, 128.94, 128.78, 128.14, 127.93, 127.83, |
| | | | 127.74, 127.27, 127.08, 126.62, 126.52, 126.26, 122.90, |
| | | | 122.40, 121.42, 121.19, 120.31, 119.37, 113.12, 112.96, |
| | | | 73.57; |
| | | | ¹⁹F NMR (471 MHz, CDCl₃) δ −58.20, −131.60 |
| J719 | | | ¹H NMR (500 MHz, CDCl₃) δ 8.54 (s, 1H), 8.47 (t, J = 8.3 Hz, |
| | | | 1H), 7.97 (d, J = 8.7 Hz, 1H), 7.89 (dd, J = 12.0, 1.7 Hz, 1H), |
| | | | 7.82-7.76 (m, 2H), 7.49 (dd, J = 10.8, 1.9 Hz, 3H), 7.39 (d, J = |
| | | | 8.6 Hz, 2H), 7.35-7.27 (m, 4H), 7.25-7.20 (m, 1H), 4.54- |
| | | | 4.42 (m, 4H), 3.87-3.61 (m, 2H); |
| | | | ¹³C NMR (126 MHz, CDCl₃) δ 171.71, 170.34, 162.29, 159.15, |
| | | | 153.00, 151.06, 148.48, 141.60, 137.42, 135.47, 134.54, |
| | | | 134.47, 134.44, 131.02, 130.14, 129.38, 128.48, 127.89, |
| | | | 127.77, 126.35, 122.91, 122.43, 121.22, 120.23, 113.12, |
| | | | 112.95, 72.63, 68.79, 32.92; |
| | | | ¹⁹FNMR(471 MHz, CDCl₃) δ −58.02, −131.07 |
| J720 | | | ¹H NMR (500 MHz, CDCl₃) δ 8.51 (s, 1H), 8.45 (q, J = 8.0 Hz, |
| | | | 1H), 8.06-8.01 (m, 1H), 8.01-7.91 (m, 2H), 7.86-7.79 (m, |
| | | | 1H), 7.78-7.73 (m, 2H), 7.65-7.61 (m, 1H), 7.60-7.50 (m, |
| | | | 3H), 7.44-7.30 (m, 5H), 4.35-4.05 (m, 2H); |
| | | | ¹³C NMR (126 MHz, CDCl₃) δ 174.80, 172.27, 170.79, 162.25, |
| | | | 159.38, 152.94, 151.00, 148.43, 141.58, 135.44, 134.54, |
| | | | 131.43, 130.41, 129.30, 128.81, 127.67, 127.60, 126.79, |
| | | | 126.63, 126.26, 126.20, 125.58, 122.86, 122.40, 121.71, |
| | | | 121.42, 121.18, 120.22, 119.36, 113.07, 74.78, 73.38; |
| | | | ¹⁹F NMR (471 MHz, CDCl₃) δ −57.94, −130.96 |
| J722 | | HRMS-ESI (m/z) | ¹H NMR (400 MHz, CDCl₃) δ 8.57 (s, 1H), 8.19 (d, J = 8.5 Hz, |
| | | [M + H]⁺ calcd for | 1H), 8.04 (d, J = 8.6 Hz, 1H), 7.99 (s, 1H), 7.74-7.64 (m, 2H), |
| | | C₃₀H₂₄F₆N₆O₄S, | 7.55 (t, J = 8.5 Hz, 1H), 7.43 (d, J = 7.8 Hz, 1H), 7.34 (d, J = |
| | | 679.1557; found, | 7.7 Hz, 2H), 7.05 (d, J = 5.6 Hz, 2H), 4.63 (d, J = 12.4 Hz, 1H), |
| | | 679.1566 | 4.51 (d, J = 12.4 Hz, 1H), 3.94 (d, J = 2.1 Hz, 2H), 3.73 (q, J = |
| | | | 8.6 Hz, 2H), 2.45 (s, 3H), 2.26 (s, 3H); |
| | | | ¹⁹F NMR (376 MHz, CDCl₃) δ −57.77, −73.75 |
| J723 | | HRMS-ESI (m/z) | ¹H NMR (400 MHz, CDCl₃) δ 8.62 (s, 1H), 8.50 (t, J = 8.4 Hz, |
| | | [M + H]⁺ calcd for | 1H), 8.04 (s, 1H), 8.01-7.86 (m, 3H), 7.73-7.64 (m, 2H), |
| | | C₂₉H₂₄F₄N₆O₃S, | 7.51 (d, J = 3.0 Hz, 1H), 7.40 (d, J = 7.8 Hz, 1H), 7.35-7.27 |
| | | 613.1639; found, | (m, 1H), 7.02 (d, J = 1.6 Hz, 1H), 4.43 (d, J = 12.5 Hz, 1H), |
| | | 613.1648 | 4.36 (d, J = 12.4 Hz, 1H), 3.95 (s, 2H), 3.47-3.34 (m, 2H), |
| | | | 2.43 (s, 3H), 1.20-1.10 (m, 3H) |
| J726 | | HRMS-ESI (m/z) | ¹H NMR (400 MHz, CDCl₃) δ 8.57 (s, 1H), 8.20 (d, J = 8.6 Hz, |
| | | [M + H]⁺ calcd for | 1H), 8.04 (d, J = 8.5 Hz, 1H), 7.99 (s, 1H), 7.74-7.63 (m, 2H), |
| | | C₃₀H₂₇F₃N₆O₄S, | 7.55 (t, J = 8.5 Hz, 1H), 7.41 (d, J = 7.7 Hz, 1H), 7.30 (d, J = |
| | | 625.1839; found, | 7.8 Hz, 1H), 7.07 (s, 1H), 7.03 (s, 1H), 4.43 (d, J = 12.5 Hz, |
| | | 625.1848 | 1H), 4.37 (d, J = 12.6 Hz, 1H), 3.93 (s, 2H), 3.43 (q, J = 7.0 Hz, |
| | | | 2H), 2.43 (s, 3H), 2.27 (s, 3H), 1.46 (s, 1H), 1.22-1.10 (m, |
| | | | 3H); |
| | | | ¹⁹F NMR (376 MHz, CDCl₃) δ −57.77 |
| J727 | | HRMS-ESI (m/z) | ¹H NMR (400 MHz, CDCl₃) δ 8.61 (s, 1H), 8.20 (d, J = 8.5 Hz, |
| | | [M + H]⁺ calcd for | 1H), 8.06 (d, J = 5.9 Hz, 2H), 8.00 (s, 1H), 7.94 (dd, J = 5.4, |
| | | C₃₀H₂₄F₆N₆O₃S, | 2.1 Hz, 1H), 7.72-7.59 (m, 2H), 7.43 (d, J = 7.8 Hz, 1H), 7.34 |
| | | 663.1608; found, | (d, J = 7.8 Hz, 1H), 7.05 (d, J = 5.1 Hz, 2H), 4.63 (d, J = 12.4 |
| | | 663.1617 | Hz, 1H), 4.51 (d, J = 12.4 Hz, 1H), 3.94 (d, J = 2.1 Hz, 2H), |
| | | | 3.73 (q, J = 8.7 Hz, 2H), 2.45 (s, 3H), 2.27 (s, 3H); |
| | | | ¹⁹F NMR (376 MHz, CDCl₃) δ −62.79, −73.75 |
| J728 | | ESIMS m/z 629 | ¹H NMR (500 MHz, CDCl₃) δ 8.57 (d, J = 9.0 Hz, 1H), 8.52 (s, |
| | | ([M + H]⁺) | 1H), 8.24-8.20 (m, 2H), 7.82 (d, J = 2.5 Hz, 1H), 7.67 (s, 1H), |
| | | | 7.60 (dd, J = 9.0, 2.5 Hz, 1H), 7.39 (d, J = 8.1 Hz, 1H), 7.36- |
| | | | 7.29 (m, 3H), 6.90 (d, J = 1.7 Hz, 1H), 4.00 (d, J = 1.5 Hz, 2H), |
| | | | 2.66 (p, J = 6.9 Hz, 1H), 2.39 (s, 3H), 1.23 (d, J = 6.8 Hz, 3H), |
| | | | 1.18 (d, J = 6.8 Hz, 3H); |
| | | | ¹⁹F NMR (471 MHz, CDCl₃) δ −57.70 |
| J729 | | ESIMS m/z 699 | ¹H NMR (500 MHz, CDCl₃) δ 8.58 (d, J = 9.0 Hz, 1H), 8.52 (s, |
| | | ([M + H]⁺) | 1H), 8.24-8.19 (m, 2H), 7.82 (d, J = 2.4 Hz, 1H), 7.68 (d, J = |
| | | | 8.0 Hz, 1H), 7.62 (dd, J = 9.1, 2.5 Hz, 1H), 7.58 (s, 1H), 7.44 |
| | | | (d, J = 7.8 Hz, 1H), 7.33-7.29 (m, 2H), 7.02 (d, J = 1.4 Hz, |
| | | | 1H), 4.50 (q, J = 6.3 Hz, 1H), 4.15-3.89 (m, 2H), 3.45 (s, 3H), |
| | | | 2.47 (s, 3H); |
| | | | ¹⁹F NMR (471 MHz, CDCl₃) δ −57.71, −76.49 |
| J730 | | ESIMS m/z 631 | ¹H NMR (500 MHz, CDCl₃) δ 8.60 (d, J = 9.0 Hz, 1H), 8.52 (s, |
| | | ([M + H]⁺) | 1H), 8.28-8.18 (m, 2H), 7.81 (d, J = 2.5 Hz, 1H), 7.66 (s, 1H), |
| | | | 7.60 (dd, J = 8.9, 2.5 Hz, 1H), 7.41 (d, J = 7.8 Hz, 1H), 7.31 (d, |
| | | | J = 8.3 Hz, 3H), 7.02 (d, J = 1.6 Hz, 1H), 4.40 (d, J = 12.6 Hz, |
| | | | 1H), 4.30 (d, J = 12.5 Hz, 1H), 4.04-3.89 (m, 2H), 3.27 (s, |
| | | | 3H), 2.44 (s, 3H); |
| | | | ¹⁹F NMR (471 MHz, CDCl₃) δ −57.70 |
| J731 | | ESIMS m/z 699 | ¹H NMR (500 MHz, CDCl₃) δ 8.59 (d, J = 9.0 Hz, 1H), 8.52 (s, |
| | | ([M + H]⁺) | 1H), 8.27-8.17 (m, 2H), 7.82 (d, J = 2.5 Hz, 1H), 7.63 (s, 1H), |
| | | | 7.61 (dd, J = 9.0, 2.5 Hz, 1H), 7.42 (d, J = 7.8 Hz, 1H), 7.38- |
| | | | 7.32 (m, 1H), 7.34-7.28 (m, 2H), 7.07-7.03 (m, 1H), 4.63 (d, |
| | | | J = 12.5 Hz, 1H), 4.51 (d, J = 12.5 Hz, 1H), 4.09-3.88 (m, |
| | | | 2H), 3.72 (qd, J = 8.7, 4.1 Hz, 2H), 2.45 (s, 3H); |
| | | | ¹⁹F NMR (471 MHz, CDCl₃) δ −57.70, −73.76 |
| J732 | | ESIMS m/z 685 | ¹H NMR (500 MHz, CDCl₃) δ 8.59 (d, J = 9.0 Hz, 1H), 8.52 (s, |
| | | ([M + H]⁺) | 1H), 8.25-8.18 (m, 2H), 7.81 (d, J = 2.5 Hz, 1H), 7.61 (d, J = |
| | | | 11.8 Hz, 2H), 7.55 (dd, J = 3.0, 1.0 Hz, 3H), 7.35-7.29 (m, |
| | | | 2H), 7.25-7.22 (m, 1H), 4.67 (d, J = 12.6 Hz, 1H), 4.58 (d, J = |
| | | | 12.5 Hz, 1H), 3.98 (d, J = 2.8 Hz, 2H), 3.75 (qd, J = 8.6, 2.1 |
| | | | Hz, 2H); |
| | | | ¹⁹F NMR (471 MHz, CDCl₃) δ −57.70, −73.78 |
| J733 | | ESIMS m/z 713 | ¹H NMR (500 MHz, CDCl₃) δ 8.59 (d, J = 9.0 Hz, 1H), 8.52 (s, |
| | | ([M + H]⁺) | 1H), 8.24-8.19 (m, 2H), 7.82 (d, J = 2.5 Hz, 1H), 7.64 (s, 1H), |
| | | | 7.60 (dd, J = 9.0, 2.5 Hz, 1H), 7.31 (dt, J = 7.8, 1.0 Hz, 2H), |
| | | | 7.28 (s, 1H), 7.00 (s, 1H), 4.61 (d, J = 12.4 Hz, 1H), 4.48 (d, J = |
| | | | 12.4 Hz, 1H), 3.96 (d, J = 2.9 Hz, 2H), 3.71 (qd, J = 8.8, 2.7 |
| | | | Hz, 2H), 2.36 (s, 3H), 2.34 (s, 3H); |
| | | | ¹⁹F NMR (471 MHz, CDCl₃) δ −57.70, −73.75 |
| J734 | | ESIMS m/z 699 | ¹H NMR (500 MHz, CDCl₃) δ 8.59 (d, J = 9.0 Hz, 1H), 8.52 (s, |
| | | ([M + H]⁺) | 1H), 8.27-8.16 (m, 2H), 7.82 (d, J = 2.5 Hz, 1H), 7.63 (s, 1H), |
| | | | 7.60 (dd, J = 9.0, 2.5 Hz, 1H), 7.34 (d, J = 6.1 Hz, 2H), 7.33- |
| | | | 7.29 (m, 2H), 7.12 (d, J = 8.5 Hz, 1H), 4.62 (d, J = 12.5 Hz, |
| | | | 1H), 4.52 (d, J = 12.4 Hz, 1H), 3.96 (d, J = 2.8 Hz, 2H), 3.86- |
| | | | 3.69 (m, 2H), 2.47 (s, 3H); |
| | | | ¹⁹F NMR (471 MHz, CDCl₃) δ −57.70, −73.76 |
| J735 | | ESIMS m/z 699 | ¹H NMR (500 MHz, CDCl₃) δ 8.59 (d, J = 9.0 Hz, 1H), 8.52 (s, |
| | | ([M + H]⁺) | 1H), 8.25-8.19 (m, 2H), 7.81 (d, J = 2.5 Hz, 1H), 7.63 (s, 1H), |
| | | | 7.60 (dd, J = 9.0, 2.5 Hz, 1H), 7.48-7.41 (m, 1H), 7.39 (d, J = |
| | | | 7.7 Hz, 1H), 7.34-7.30 (m, 2H), 7.06 (dd, J = 8.0, 1.5 Hz, 1H), |
| | | | 4.61 (s, 2H), 3.98 (d, J = 2.7 Hz, 2H), 3.74 (qd, J = 8.8, 5.6 Hz, |
| | | | 2H), 2.51 (s, 3H); |
| | | | ¹⁹F NMR (471 MHz, CDCl₃) δ −57.70, −73.62 |
| J736 | | ESIMS m/z 681 | ¹H NMR (500 MHz, CDCl₃) δ 8.59 (d, J = 9.0 Hz, 1H), 8.52 (s, |
| | | ([M + H]⁺) | 1H), 8.23-8.19 (m, 2H), 7.82 (d, J = 2.5 Hz, 1H), 7.64 (s, 1H), |
| | | | 7.60 (dd, J = 9.0, 2.5 Hz, 1H), 7.40 (d, J = 7.9 Hz, 1H), 7.36- |
| | | | 7.30 (m, 3H), 7.04 (d, J = 1.6 Hz, 1H), 5.80 (tt, J = 55.3, 4.0 Hz, |
| | | | 1H), 4.57 (d, J = 12.5 Hz, 1H), 4.45 (d, J = 12.4 Hz, 1H), 4.05- |
| | | | 3.87 (m, 2H), 3.57 (td, J = 14.0, 4.1 Hz, 2H), 2.45 (s, 3H); |
| | | | ¹⁹F NMR (471 MHz, CDCl₃) δ −57.70, −124.91 (d, J = 25.9 Hz) |
| J737 | | ESIMS m/z 699 | ¹H NMR (500 MHz, CDCl₃) δ 8.61 (d, J = 9.0 Hz, 1H), 8.52 (s, |
| | | ([M + H]⁺) | 1H), 8.28-8.16 (m, 2H), 7.81 (d, J = 2.5 Hz, 1H), 7.66 (s, 1H), |
| | | | 7.60 (dd, J = 9.0, 2.5 Hz, 1H), 7.33-7.29 (m, 2H), 7.29-7.27 |
| | | | (m, 1H), 7.03 (d, J = 2.2 Hz, 1H), 6.97 (d, J = 8.4 Hz, 1H), 4.23 |
| | | | (td, J = 6.3, 3.2 Hz, 2H), 3.94 (d, J = 2.4 Hz, 2H), 2.54 (dtd, J = |
| | | | 16.3, 10.5, 6.1 Hz, 2H), 2.38 (s, 3H); |
| | | | ¹⁹F NMR (471 MHz, CDCl₃) δ −57.70, −64.57 |
| J738 | | HRMS-ESI (m/z) | ¹H NMR (400 MHz, CDCl₃) δ 8.57 (s, 1H), 8.49 (t, J = 8.4 Hz, |
| | | [M + H]⁺ calcd for | 1H), 7.98 (d, J = 8.6 Hz, 1H), 7.89 (dd, J = 12.1, 1.8 Hz, 1H), |
| | | C₃₀H₂₃F₇N₆O₄S, | 7.72-7.62 (m, 2H), 7.56 (t, J = 8.5 Hz, 1H), 7.49 (d, J = 3.0 |
| | | 697.1462; found, | Hz, 1H), 7.28 (s, 1H), 6.99 (s, 1H), 4.60 (d, J = 12.3 Hz, 1H), |
| | | 697.1467 | 4.47 (d, J = 12.3 Hz, 1H), 3.95 (d, J = 2.5 Hz, 2H), 3.71 (qd, J = |
| | | | 8.7, 4.2 Hz, 3H), 2.35 (d, J = 10.1 Hz, 6H); |
| | | | ¹⁹F NMR (376 MHz, CDCl₃) δ −57.79, −73.77, −131.45 |
| J740 | | | ¹H NMR (400 MHz, CDCl₃) δ 8.54 (s, 1H), 8.47 (t, J = 8.3 Hz, |
| | | | 1H), 7.97 (d, J = 8.6 Hz, 1H), 7.89 (dd, J = 12.0, 1.6 Hz, 1H), |
| | | | 7.82-7.74 (m, 2H), 7.65 (dd, J = 8.2, 1.9 Hz, 1H), 7.49 (d, J = |
| | | | 2.7 Hz, 1H), 7.44 (d, J = 8.3 Hz, 1H), 7.42-7.36 (m, 3H), 7.34- |
| | | | 7.27 (m, 4H), 4.58-4.33 (m, 4H), 3.83 (d, J = 18.1 Hz, 1H), |
| | | | 3.64 (d, J = 18.1 Hz, 1H); |
| | | | ¹³C NMR (126 MHz, CDCl₃) δ 171.70, 171.16, 170.32, 162.29, |
| | | | 159.13, 153.00, 151.06, 148.45, 141.61, 137.40, 135.46, |
| | | | 135.01, 134.67, 133.06, 132.16, 131.25, 128.47, 127.89, |
| | | | 127.84, 127.76, 127.70, 126.34, 126.27, 122.88, 122.42, |
| | | | 122.04, 121.42, 121.20, 120.23, 119.37, 113.12, 112.95, |
| | | | 72.63, 68.81, 32.91; |
| | | | ¹⁹F NMR (376 MHz, CDCl₃) δ −58.47, −130.99 |
| J741 | | | ¹H NMR (400 MHz, CDCl₃) δ 8.54 (s, 1H), 8.46 (t, J = 8.3 Hz, |
| | | | 1H), 7.97 (d, J = 8.6 Hz, 1H), 7.89 (dd, J = 12.0, 1.6 Hz, 1H), |
| | | | 7.82-7.69 (m, 4H), 7.51-7.43 (m, 2H), 7.38 (d, J = 8.6 Hz, |
| | | | 2H), 7.35-7.28 (m, 5H), 4.60-4.43 (m, 4H), 3.84 (s, 1H), |
| | | | 3.69 (s, 1H); |
| | | | ¹³C NMR (126 MHz, CDCl₃) δ 171.67, 171.16, 170.28, 162.28, |
| | | | 159.04, 153.00, 151.06, 148.46, 141.61, 140.10, 137.23, |
| | | | 135.45, 133.84, 131.63, 131.36, 130.31, 128.51, 127.99, |
| | | | 127.79, 127.72, 127.64, 126.80, 126.40, 126.34, 124.46, |
| | | | 122.91, 122.42, 122.29, 121.42, 121.21, 120.23, 119.37, |
| | | | 113.12, 112.95, 72.90, 68.77, 60.40; |
| | | | ¹⁹F NMR (376 MHz, CDCl₃) δ −58.02, −61.45, −130.97 |
| J742 | | | ¹H NMR (400 MHz, CDCl₃) δ 8.54 (s, 1H), 8.47 (t, J = 8.4 Hz, |
| | | | 1H), 7.97 (d, J = 9.0 Hz, 1H), 7.88 (dd, J = 12.0, 1.6 Hz, 1H), |
| | | | 7.82-7.74 (m, 2H), 7.52 (dddd, J = 20.8, 15.7, 10.5, 2.6 Hz, |
| | | | 4H), 7.38 (d, J = 8.6 Hz, 2H), 7.34-7.28 (m, 4H), 7.24-7.18 |
| | | | (m, 1H), 4.55-4.41 (m, 4H), 3.84 (dd, J = 18.0, 4.0 Hz, 1H), |
| | | | 3.65 (d, J = 18.0 Hz, 1H); |
| | | | ¹³C NMR (126 MHz, CDCl₃) δ 172.23, 170.90, 162.42, 159.50, |
| | | | 153.09, 151.15, 148.58, 141.72, 137.77, 135.82, 135.59, |
| | | | 133.88, 130.24, 130.04, 129.50, 129.33, 129.22, 128.55, |
| | | | 128.23, 128.05, 128.00, 127.90, 126.33, 126.27, 123.01, |
| | | | 122.54, 121.55, 121.33, 120.35, 119.49, 113.21, 113.04, |
| | | | 72.68, 69.57, 33.07; |
| | | | ¹⁹F NMR (376 MHz, CDCl₃) δ −58.02, −131.11 |
| J744 | | | ¹H NMR (400 MHz, Acetone-d₆) δ 9.60 (s, 1H), 9.41 (s, 1H), |
| | | | 8.07 (d, J = 8.6 Hz, 2H), 7.95-7.78 (m, 4H), 7.73 (s, 2H), 7.62 |
| | | | (d, J = 8.7 Hz, 2H), 7.50 (s, 3H), 7.42 (d, J = 5.9 Hz, 2H), 4.92 |
| | | | (d, J = 8.8 Hz, 1H), 4.10 (s, 1H); |
| | | | ¹⁹F NMR (376 MHz, Acetone-d₆) δ −58.33, −116.38 |
| J745 | | | ¹H NMR (400 MHz, CDCl₃) δ 8.54 (d, J = 0.8 Hz, 1H), 8.42 (tt, |
| | | | J = 13.2, 7.0 Hz, 1H), 7.94 (d, J = 8.2 Hz, 1H), 7.86 (d, J = 12.0 |
| | | | Hz, 1H), 7.78 (d, J = 8.9 Hz, 2H), 7.68 (dd, J = 6.0, 2.9 Hz, 1H), |
| | | | 7.57 (d, J = 15.2 Hz, 1H), 7.49 (td, J = 8.0, 4.3 Hz, 2H), 7.39 (d, |
| | | | J = 8.8 Hz, 2H), 7.36-7.28 (m, 3H), 7.08-6.90 (m, 1H), 4.70- |
| | | | 4.55 (m, 4H), 4.17-3.89 (m, 2H), 2.43 (d, J = 5.9 Hz, 3H); |
| | | | ¹³C NMR (126 MHz, CDCl₃) δ 174.62, 172.42, 171.24, 169.91, |
| | | | 162.31, 162.25, 159.30, 150.81, 148.46, 141.58, 140.76, |
| | | | 140.14, 135.46, 131.44, 130.84, 130.64, 129.64, 125.44, |
| | | | 125.41, 124.51, 122.83, 122.42, 121.21, 120.23, 112.87, |
| | | | 110.81, 73.37, 72.97, 69.77, 69.45, 64.04, 57.68, 33.16, 27.54, |
| | | | 25.20; |
| | | | ¹⁹F NMR (376 MHz, CDCl₃) δ −56.42, −130.46 |
| J746 | | | ¹H NMR (400 MHz, CDCl₃) δ 8.63 (s, 1H), 8.45 (t, J = 8.3 Hz, |
| | | | 1H), 7.97 (d, J = 8.6 Hz, 1H), 7.89 (t, J = 6.8 Hz, 2H), 7.80 (d, J = |
| | | | 8.6 Hz, 2H), 7.46 (d, J = 7.6 Hz, 3H), 7.32 (dd, J = 19.2, 7.4 |
| | | | Hz, 1H), 7.25-7.20 (m, 2H), 7.16 (s, 1H), 7.02 (s, 1H), 4.52 |
| | | | (dd, J = 7.2, 4.7 Hz, 4H), 4.03-3.73 (m, 2H), 2.45 (s, 2H); |
| | | | ¹³C NMR (126 MHz, CDCl₃) δ 172.16, 170.87, 159.32, 152.93, |
| | | | 146.63, 141.65, 139.66, 139.46, 133.60, 132.26, 130.80, |
| | | | 130.70, 130.09, 129.52, 129.09, 128.76, 128.01, 127.16, |
| | | | 127.13, 126.89, 125.92, 122.92, 120.26, 120.19, 119.56, |
| | | | 113.11, 112.94, 69.81, 66.45, 33.00, 21.11; |
| | | | ¹⁹F NMR (376 MHz, CDCl₃) δ −57.24, −62.27, −129.93 |
| J747 | | | ¹H NMR (400 MHz, CDCl₃) δ 8.63 (s, 1H), 8.49 (t, J = 8.4 Hz, |
| | | | 1H), 7.98 (d, J = 8.7 Hz, 1H), 7.94-7.86 (m, 3H), 7.79 (d, J = |
| | | | 8.6 Hz, 2H), 7.48 (d, J = 2.4 Hz, 1H), 7.43 (d, J = 7.8 Hz, 1H), |
| | | | 7.36-7.27 (m, 6H), 7.02 (s, 1H), 4.53-4.39 (m, 4H), 3.87- |
| | | | 3.57 (m, 2H), 2.42 (d, J = 19.2 Hz, 3H); |
| | | | ¹³C NMR (126 MHz, CDCl₃) δ 172.24, 170.88, 162.52, 159.43, |
| | | | 152.95, 151.01, 141.66, 139.49, 137.75, 133.68, 132.49, |
| | | | 130.71, 130.12, 129.86, 129.52, 128.40, 128.06, 127.98, |
| | | | 127.73, 127.15, 127.12, 125.98, 124.69, 122.98, 120.22, |
| | | | 119.56, 113.14, 112.97, 72.34, 69.31, 32.94, 24.87; |
| | | | ¹⁹F NMR (376 MHz, CDCl₃) δ −62.52, −131.03 |
| J748 | | | ¹H NMR (400 MHz, CDCl₃) δ 8.72-8.40 (m, 2H), 7.89 (d, J = |
| | | | 8.6 Hz, 4H), 7.79 (t, J = 6.5 Hz, 5H), 7.74 (s, 1H), 7.42 (d, J = |
| | | | 7.8 Hz, 1H), 7.05 (s, 1H), 6.85 (t, J = 8.6 Hz, 1H), 4.50 (d, J = |
| | | | 11.2 Hz, 2H), 4.04-3.68 (m, 4H), 2.43 (d, J = 20.2 Hz, 3H); |
| | | | ¹³C NMR (126 MHz, CDCl₃) δ 172.21, 170.83, 163.17, 159.22, |
| | | | 152.33, 150.44, 141.66, 141.41, 140.60, 140.14, 139.58, |
| | | | 136.29, 136.18, 133.88, 131.74, 130.87, 130.22, 129.85, |
| | | | 129.74, 129.58, 127.13, 127.10, 127.07, 124.75, 123.15, |
| | | | 123.13, 122.98, 121.45, 120.98, 120.92, 120.17, 119.56, |
| | | | 119.41, 116.55, 116.53, 113.81, 113.65, 113.12, 112.95, |
| | | | 70.35, 70.07, 32.99 |
| J749 | | | ¹H NMR (400 MHz, CDCl₃) δ 8.64 (s, 1H), 8.44 (t, J = 8.4 Hz, |
| | | | 1H), 7.96 (d, J = 9.1 Hz, 1H), 7.93-7.85 (m, 3H), 7.80 (d, J = |
| | | | 8.5 Hz, 2H), 7.47 (d, J = 7.8 Hz, 1H), 7.42 (d, J = 8.4 Hz, 2H), |
| | | | 7.34 (d, J = 7.9 Hz, 1H), 7.23-7.17 (m, 1H), 7.12 (t, J = 6.9 |
| | | | Hz, 1H), 7.03 (s, 1H), 5.30 (s, 1H), 4.57-4.47 (m, 4H), 3.86 |
| | | | (d, J = 15.7 Hz, 1H), 2.45 (s, 3H); |
| | | | ¹³C NMR (126 MHz, CDCl₃) δ 172.18, 171.11, 161.48, 158.98, |
| | | | 141.65, 140.14, 135.60, 134.12, 132.56, 132.34, 130.81, |
| | | | 130.20, 129.56, 129.12, 128.59, 128.49, 127.16, 126.79, |
| | | | 120.18, 119.56, 113.11, 69.93, 69.32, 33.04, 23.37; |
| | | | ¹⁹F NMR (376 MHz, CDCl₃) δ −62.27, −131.02 |
| J750 | | ESIMS m/z 698 | ¹H NMR (400 MHz, CDCl₃) δ 8.67 (s, 1H), 8.49 (t, J = 8.4 Hz, |
| | | ([M + H]⁺) | 1H), 8.27 (d, J = 5.7 Hz, 1H), 7.97 (d, J = 8.6 Hz, 1H), 7.89 (dd, |
| | | | J = 11.9, 1.9 Hz, 1H), 7.48 (d, J = 3.1 Hz, 1H), 7.45-7.37 (m, |
| | | | 2H), 7.37-7.32 (m, 1H), 7.29 (d, J = 1.8 Hz, 1H), 7.04 (d, J = |
| | | | 1.8 Hz, 1H), 4.83 (q, J = 8.5 Hz, 2H), 4.63 (d, J = 12.4 Hz, 1H), |
| | | | 4.50 (d, J = 12.4 Hz, 1H), 3.96 (d, J = 2.8 Hz, 2H), 3.71 (qd, J = |
| | | | 8.7, 4.2 Hz, 2H), 2.45 (s, 3H); |
| | | | ¹⁹F NMR (376 MHz, CDCl₃) δ −73.78, −73.82, −131.22 |
| J751 | | ESIMS m/z 699 | ¹H NMR (400 MHz, CDCl₃) δ 8.61 (s, 1H), 8.49 (t, J = 8.3 Hz, |
| | | ([M + H]⁺) | 1H), 7.95 (d, J = 8.6 Hz, 1H), 7.86 (dd, J = 11.9, 1.8 Hz, 1H), |
| | | | 7.49 (d, J = 3.1 Hz, 1H), 7.43 (dd, J = 14.7, 7.7 Hz, 2H), 7.35 |
| | | | (d, J = 7.9 Hz, 1H), 7.04 (d, J = 1.6 Hz, 1H), 6.95 (d, J = 2.4 |
| | | | Hz, 1H), 6.84 (dd, J = 7.6, 2.5 Hz, 1H), 4.71-4.58 (m, 3H), |
| | | | 4.50 (d, J = 12.4 Hz, 1H), 3.96 (d, J = 2.7 Hz, 2H), 3.71 (qd, J = |
| | | | 8.7, 3.9 Hz, 2H), 2.45 (s, 3H); |
| | | | ¹⁹F NMR (376 MHz, CDCl₃) δ −70.60, −73.78, −131.10 |
| J752 | | HRMS-ESI (m/z) | ¹H NMR (400 MHz, CDCl₃) δ 8.50 (s, 1H), 8.46 (t, J = 8.4 Hz, |
| | | [M + H]⁺ calcd for | 1H), 7.97 (d, J = 8.6 Hz, 1H), 7.89 (dd, J = 12.1, 1.8 Hz, 1H), |
| | | C₂₉H₂₄F₄N₆O₃S, | 7.63-7.57 (m, 2H), 7.46 (d, J = 3.1 Hz, 1H), 7.41 (d, J = 7.8 |
| | | 613.1639 found, | Hz, 1H), 7.36-7.28 (m, 3H), 7.06-7.01 (m, 1H), 4.63 (d, J = |
| | | 613.165 | 12.5 Hz, 1H), 4.50 (d, J = 12.4 Hz, 1H), 3.95 (d, J = 2.8 Hz, |
| | | | 2H), 3.71 (qd, J = 8.7, 4.4 Hz, 2H), 2.43 (d, J = 10.3 Hz, 7H); |
| | | | ¹⁹F NMR (376 MHz, CDCl₃) δ −73.78, −131.56 |
| J753 | | HRMS-ESI (m/z) | ¹H NMR (400 MHz, CDCl₃) δ 8.50 (s, 1H), 8.46 (d, J = 8.3 Hz, |
| | | [M + H]⁺ calcd for | 1H), 8.01-7.93 (m, 1H), 7.89 (dd, J = 12.1, 1.8 Hz, 1H), 7.63- |
| | | C₂₉H₂₇FN₆O₃S, | 7.57 (m, 2H), 7.48 (d, J = 2.6 Hz, 1H), 7.40 (d, J = 7.7 Hz, |
| | | 559.1922; found, | 1H), 7.30 (dd, J = 8.1, 4.4 Hz, 3H), 7.01 (d, J = 1.5 Hz, 1H), |
| | | 559.1929 | 4.43 (d, J = 12.6 Hz, 1H), 4.35 (d, J = 12.5 Hz, 1H), 3.99-3.88 |
| | | | (m, 2H), 3.41 (q, J = 7.0 Hz, 2H), 2.42 (d, J = 3.1 Hz, 6H), 1.20- |
| | | | 1.08 (m, 3H); |
| | | | ¹⁹F NMR (376 MHz, CDCl₃) δ −131.37 |
| J754 | | ESIMS m/z 629 | ¹H NMR (400 MHz, CDCl₃) δ 8.47 (d, J = 8.4 Hz, 1H), 8.44 (s, |
| | | ([M + H]⁺) | 1H), 7.96 (d, J = 8.6 Hz, 1H), 7.88 (dd, J = 12.2, 1.8 Hz, 1H), |
| | | | 7.65-7.58 (m, 2H), 7.45 (s, 1H), 7.41 (d, J = 7.9 Hz, 1H), 7.34 |
| | | | (d, J = 7.8 Hz, 1H), 7.04 (d, J = 2.8 Hz, 2H), 7.01 (d, J = 2.3 |
| | | | Hz, 1H), 4.63 (d, J = 12.4 Hz, 1H), 4.50 (d, J = 12.4 Hz, 1H), |
| | | | 3.95 (d, J = 2.8 Hz, 2H), 3.87 (s, 3H), 3.71 (qd, J = 8.8, 4.4 Hz, |
| | | | 2H), 2.45 (s, 3H); |
| | | | ¹⁹F NMR (376 MHz, CDCl₃) δ −73.78, −131.58 |
| J755 | | ESIMS m/z 575 | ¹H NMR (400 MHz, CDCl₃) δ 8.47 (t, J = 8.4 Hz, 1H), 8.43 (s, |
| | | ([M + H]⁺) | 1H), 7.96 (d, J = 8.6 Hz, 1H), 7.88 (dd, J = 12.2, 1.9 Hz, 1H), |
| | | | 7.66-7.58 (m, 2H), 7.48 (s, 1H), 7.40 (d, J = 7.8 Hz, 1H), 7.30 |
| | | | (d, J = 8.0 Hz, 1H), 7.05-6.99 (m, 3H), 4.43 (d, J = 12.7 Hz, |
| | | | 1H), 4.35 (d, J = 12.6 Hz, 1H), 3.99-3.91 (m, 2H), 3.87 (s, |
| | | | 3H), 3.41 (q, J = 7.0 Hz, 2H), 2.43 (s, 3H), 1.15 (t, J = 7.0 Hz, |
| | | | 3H); |
| | | | ¹⁹F NMR (376 MHz, CDCl₃) δ −131.38 |
| J756 | | ESIMS m/z 643 | ¹H NMR (400 MHz, CDCl₃) δ 8.47 (t, J = 8.4 Hz, 1H), 8.44 (s, |
| | | ([M + H]⁺) | 1H), 7.96 (d, J = 8.6 Hz, 1H), 7.88 (dd, J = 12.1, 1.9 Hz, 1H), |
| | | | 7.65-7.58 (m, 2H), 7.47 (s, 1H), 7.28 (s, 1H), 7.05-6.96 (m, |
| | | | 3H), 4.60 (d, J = 12.4 Hz, 1H), 4.47 (d, J = 12.4 Hz, 1H), 3.94 |
| | | | (d, J = 2.5 Hz, 2H), 3.87 (s, 3H), 3.71 (qd, J = 8.7, 3.4 Hz, 2H), |
| | | | 2.35 (d, J = 10.1 Hz, 6H); |
| | | | ¹⁹F NMR (376 MHz, CDCl₃) δ −73.78, −131.66 |
| J757 | | ESIMS m/z 611 | ¹H NMR (400 MHz, CDCl₃) δ 8.47 (d, J = 8.3 Hz, 1H), 8.44 (s, |
| | | ([M + H]⁺) | 1H), 7.96 (d, J = 8.6 Hz, 1H), 7.88 (dd, J = 12.1, 1.8 Hz, 1H), |
| | | | 7.65-7.58 (m, 2H), 7.46 (s, 1H), 7.40 (d, J = 7.8 Hz, 1H), 7.33 |
| | | | (d, J = 7.8 Hz, 1H), 7.02 (d, J = 8.9 Hz, 3H), 5.80 (tt, J = 55.3, |
| | | | 4.0 Hz, 1H), 4.56 (d, J = 12.4 Hz, 1H), 4.44 (d, J = 12.3 Hz, |
| | | | 1H), 4.02-3.90 (m, 2H), 3.87 (s, 3H), 3.56 (td, J = 13.9, 4.1 |
| | | | Hz, 2H), 2.44 (s, 3H); |
| | | | ¹⁹F NMR (376 MHz, CDCl₃) δ −124.91, −131.52 |
| J758 | | ESIMS m/z 646 | ¹H NMR (400 MHz, CDCl₃) δ 8.64 (s, 1H), 8.52-8.38 (m, 3H), |
| | | ([M + H]⁺) | 7.99 (d, J = 8.6 Hz, 1H), 7.96-7.86 (m, 3H), 7.80 (d, J = 8.5 |
| | | | Hz, 2H), 7.47 (d, J = 3.0 Hz, 1H), 7.38 (d, J = 7.9 Hz, 1H), 7.33- |
| | | | 7.26 (m, 2H), 7.16 (dd, J = 7.8, 4.8 Hz, 1H), 6.99-6.93 (m, |
| | | | 1H), 3.89-3.59 (m, 4H), 2.42 (s, 3H); |
| | | | ¹⁹F NMR (376 MHz, CDCl₃) δ −62.52, −131.08 |
| J759 | | ESIMS m/z 662 | ¹H NMR (400 MHz, CDCl₃) δ 8.55 (s, 1H), 8.51-8.42 (m, 2H), |
| | | ([M + H]⁺) | 8.43 (dd, J = 4.7, 1.7 Hz, 1H), 7.98 (d, J = 8.6 Hz, 1H), 7.91 |
| | | | (dd, J = 12.0, 1.9 Hz, 1H), 7.83-7.75 (m, 2H), 7.46 (d, J = 3.0 |
| | | | Hz, 1H), 7.42-7.35 (m, 3H), 7.34-7.25 (m, 2H), 7.16 (dd, J = |
| | | | 7.8, 4.7 Hz, 1H), 6.96 (s, 1H), 3.89-3.62 (m, 4H), 2.42 (s, 3H); |
| | | | ¹⁹F NMR (376 MHz, CDCl₃) δ −58.02, −131.14 |
| J760 | | HRMS-ESI (m/z) | ¹H NMR (400 MHz, CDCl₃) δ 8.69 (s, 1H), 8.51 (t, J = 8.4 Hz, |
| | | [M + H]⁺ calcd for | 1H), 8.23 (d, J = 1.6 Hz, 2H), 8.06-7.95 (m, 1H), 7.95-7.84 |
| | | C₃₀H₂₀F₁₀N₆O₃S, | (m, 2H), 7.48 (d, J = 3.0 Hz, 1H), 7.42 (d, J = 7.8 Hz, 1H), 7.37- |
| | | 735.1231; found, | 7.29 (m, 1H), 7.04 (s, 1H), 4.63 (d, J = 12.4 Hz, 1H), 4.50 (d, |
| | | 735.1239 | J = 12.4 Hz, 1H), 3.96 (d, J = 2.8 Hz, 2H), 3.72 (qd, J = 8.7, 4.0 |
| | | | Hz, 2H), 2.45 (s, 3H); |
| | | | ¹⁹F NMR (376 MHz, CDCl₃) δ −62.99, −73.79, −131.17 |
| J761 | | HRMS-ESI (m/z) | ¹H NMR (400 MHz, CDCl₃) δ 8.69 (s, 1H), 8.51 (t, J = 8.4 Hz, |
| | | [M + H]⁺ calcd for | 1H), 8.23 (d, J = 1.4 Hz, 2H), 8.04-7.95 (m, 1H), 7.92 (d, J = |
| | | C₃₀H₂₃F₇N₆O₃S, | 11.5 Hz, 2H), 7.51 (d, J = 3.0 Hz, 1H), 7.40 (d, J = 7.8 Hz, 1H), |
| | | 681.1513; found, | 7.34-7.28 (m, 1H), 7.01 (d, J = 1.6 Hz, 1H), 4.43 (d, J = 12.6 |
| | | 681.1521 | Hz, 1H), 4.36 (d, J = 12.5 Hz, 1H), 3.95 (d, J = 0.9 Hz, 2H), |
| | | | 3.41 (q, J = 7.0 Hz, 2H), 2.43 (s, 3H), 1.15 (t, J = 7.0 Hz, 3H); |
| | | | ¹⁹F NMR (376 MHz, CDCl₃) δ −62.99, −130.96 |
| J762 | | HRMS-ESI (m/z) | ¹H NMR (400 MHz, CDCl₃) δ 8.69 (s, 1H), 8.26-8.18 (m, 3H), |
| | | [M + H]⁺ calcd for | 8.06 (d, J = 8.7 Hz, 1H), 8.01 (s, 1H), 7.89 (s, 1H), 7.43 (d, J = |
| | | C₃₁H₂₃F₉N₆O₃S, | 7.8 Hz, 1H), 7.34 (d, J = 7.7 Hz, 1H), 7.06 (s, 2H), 4.63 (d, J = |
| | | 731.1481; found, | 12.4 Hz, 1H), 4.51 (d, J = 12.4 Hz, 1H), 3.95 (d, J = 2.1 Hz, |
| | | 731.149 | 2H), 3.73 (q, J = 8.7 Hz, 2H), 2.45 (s, 3H), 2.28 (s, 3H); |
| | | | ¹⁹F NMR (376 MHz, CDCl₃) δ −62.96, −73.76 |
| J763 | | HRMS-ESI (m/z) | ¹H NMR (400 MHz, CDCl₃) δ 8.69 (s, 1H), 8.23 (d, J = 7.3 Hz, |
| | | [M + H]⁺ calcd for | 3H), 8.05 (d, J = 8.6 Hz, 1H), 8.01 (s, 1H), 7.89 (s, 1H), 7.41 (d, |
| | | C₃₁H₂₆F₆N₆O₃S, | J = 7.8 Hz, 1H), 7.30 (d, J = 7.8 Hz, 1H), 7.09 (s, 1H), 7.03 (s, |
| | | 677.1764; found, | 1H), 4.47-4.33 (m, 2H), 3.94 (s, 2H), 3.43 (q, J = 7.0 Hz, 2H), |
| | | 677.1771 | 2.43 (s, 3H), 2.28 (s, 3H), 1.17 (t, J = 7.0 Hz, 3H); |
| | | | ¹⁹F NMR (376 MHz, CDCl₃) δ −62.96 |
| J764 | | HRMS-ESI (m/z) | ¹H NMR (400 MHz, CDCl₃) δ 8.47 (s, 2H), 7.96 (d, J = 8.7 Hz, |
| | | [M + H]⁺ calcd for | 1H), 7.88 (dd, J = 12.1, 1.8 Hz, 1H), 7.72-7.65 (m, 2H), 7.46 |
| | | C₃₀H₂₃F₇N₆O₄S, | (d, J = 3.0 Hz, 1H), 7.42 (d, J = 7.8 Hz, 1H), 7.38-7.31 (m, |
| | | 697.1462; found, | 1H), 7.12-7.05 (m, 2H), 7.04 (s, 1H), 4.63 (d, J = 12.4 Hz, |
| | | 697.1465 | 1H), 4.50 (d, J = 12.4 Hz, 1H), 4.42 (q, J = 8.0 Hz, 2H), 3.96 (d, |
| | | | J = 2.8 Hz, 2H), 3.71 (qd, J = 8.7, 4.4 Hz, 2H), 2.45 (s, 3H) |
| J765 | | ESIMS m/z 699 | ¹H NMR (400 MHz, CDCl₃) δ 8.54-8.44 (m, 3H), 8.03 (dd, J = |
| | | ([M + H]⁺) | 8.8, 2.7 Hz, 1H), 7.99-7.93 (m, 1H), 7.87 (dd, J = 12.1, 1.9 |
| | | | Hz, 1H), 7.48 (d, J = 3.0 Hz, 1H), 7.42 (d, J = 7.8 Hz, 1H), 7.35 |
| | | | (d, J = 7.9 Hz, 1H), 7.08-7.01 (m, 2H), 4.82 (q, J = 8.4 Hz, |
| | | | 2H), 4.63 (d, J = 12.4 Hz, 1H), 4.50 (d, J = 12.4 Hz, 1H), 3.96 |
| | | | (d, J = 2.7 Hz, 2H), 3.71 (qd, J = 8.7, 4.0 Hz, 2H), 2.45 (s, 3H); |
| | | | ¹⁹F NMR (376 MHz, CDCl₃) δ −73.76, −73.78, −131.31 |
| J766 | | HRMS-ESI (m/z) | ¹H NMR (400 MHz, CDCl₃) δ 8.52-8.44 (m, 2H), 7.96 (d, J = |
| | | [M + H]⁺ calcd for | 8.6 Hz, 1H), 7.88 (dd, J = 12.0, 1.8 Hz, 1H), 7.73-7.64 (m, |
| | | C₃₀H₂₆F₄N₆O₄S, | 2H), 7.49 (d, J = 3.1 Hz, 1H), 7.40 (d, J = 7.8 Hz, 1H), 7.30 (d, |
| | | 643.1745; found, | J = 7.4 Hz, 1H), 7.13-7.04 (m, 2H), 7.01 (s, 1H), 4.47-4.32 |
| | | 643.1749 | (m, 4H), 4.04-3.82 (m, 2H), 3.41 (q, J = 7.0 Hz, 2H), 2.43 (s, |
| | | | 3H), 1.20-1.10 (m, 3H); |
| | | | ¹⁹F NMR (376 MHz, CDCl₃) δ −73.83, −131.27 |
| J767 | | ESIMS m/z 666 | ¹H NMR (400 MHz, CDCl₃) δ 8.29-8.22 (m, 2H), 8.18 (d, J = |
| | | ([M + H]⁺) | 8.6 Hz, 2H), 7.66 (d, J = 8.6 Hz, 2H), 7.43 (d, J = 7.9 Hz, 3H), |
| | | | 7.35 (d, J = 6.9 Hz, 2H), 7.05 (s, 1H), 4.62 (d, J = 12.4 Hz, 1H), |
| | | | 4.51 (d, J = 12.3 Hz, 1H), 3.96 (d, J = 2.2 Hz, 2H), 3.81-3.65 |
| | | | (m, 2H), 2.45 (s, 3H); |
| | | | ¹⁹F NMR (376 MHz, CDCl₃) δ −57.91, −73.76 |
| J768 | | ESIMS m/z 648 | ¹H NMR (400 MHz, CDCl₃) δ 8.33-8.21 (m, 2H), 8.18 (d, J = |
| | | ([M + H]⁺) | 8.6 Hz, 2H), 7.66 (d, J = 8.7 Hz, 2H), 7.42 (dd, J = 8.4, 3.7 Hz, |
| | | | 3H), 7.34 (d, J = 9.2 Hz, 2H), 7.05 (s, 1H), 6.01-5.63 (m, 1H), |
| | | | 4.56 (d, J = 12.3 Hz, 1H), 4.45 (d, J = 12.3 Hz, 1H), 4.07-3.85 |
| | | | (m, 2H), 3.58 (td, J = 14.0, 4.1 Hz, 2H), 2.44 (s, 3H); |
| | | | ¹⁹F NMR (376 MHz, CDCl₃) δ −57.91, −124.89 (d, J = 12.5 Hz) |
| J769 | | HRMS-ESI (m/z) | ¹H NMR (400 MHz, CDCl₃) δ 8.55 (s, 1H), 8.47 (t, J = 8.4 Hz, |
| | | [M + H]⁺ calcd for | 1H), 7.98 (d, J = 8.6 Hz, 1H), 7.90 (dd, J = 12.1, 1.8 Hz, 1H), |
| | | C₂₈H₂₂F₄N₆O₃S, | 7.76-7.70 (m, 2H), 7.53 (t, J = 7.9 Hz, 2H), 7.47 (d, J = 3.1 |
| | | 599.1483; found, | Hz, 1H), 7.45-7.38 (m, 2H), 7.35 (d, J = 7.9 Hz, 1H), 7.04 (d, |
| | | 599.149 | J = 1.6 Hz, 1H), 4.63 (d, J = 12.4 Hz, 1H), 4.50 (d, J = 12.5 Hz, |
| | | | 1H), 3.96 (d, J = 2.8 Hz, 2H), 3.71 (qd, J = 8.7, 4.4 Hz, 2H), |
| | | | 2.45 (s, 3H); |
| | | | ¹⁹F NMR (376 MHz, CDCl₃) δ −73.78, −131.51 |
| J770 | | ESIMS m/z 684 | ¹H NMR (400 MHz, CDCl₃) δ 8.55 (t, J = 8.3 Hz, 1H), 8.26 (d, J = |
| | | ([M + H]⁺) | 8.9 Hz, 2H), 7.96 (d, J = 8.6 Hz, 1H), 7.86 (d, J = 11.6 Hz, |
| | | | 1H), 7.53 (d, J = 5.7 Hz, 1H), 7.39 (q, J = 9.8, 8.9 Hz, 4H), 7.04 |
| | | | (s, 1H), 4.62 (d, J = 12.4 Hz, 1H), 4.50 (d, J = 12.4 Hz, 1H), |
| | | | 3.97 (d, J = 2.8 Hz, 2H), 3.71 (qd, J = 8.7, 3.9 Hz, 2H), 2.45 (s, |
| | | | 3H); |
| | | | ¹⁹F NMR (376 MHz, CDCl₃) δ −57.61, −73.78, −130.71 |
| J771 | | ESIMS m/z 666 | ¹H NMR (400 MHz, CDCl₃) δ 8.55 (t, J = 8.3 Hz, 1H), 8.26 (d, J = |
| | | ([M + H]⁺) | 8.8 Hz, 2H), 7.95 (d, J = 8.6 Hz, 1H), 7.91-7.82 (m, 1H), |
| | | | 7.53 (d, J = 8.5 Hz, 1H), 7.47-7.37 (m, 3H), 7.34 (d, J = 7.8 |
| | | | Hz, 1H), 7.03 (s, 1H), 5.80 (tt, J = 55.0, 4.1 Hz, 1H), 4.56 (d, J = |
| | | | 12.4 Hz, 1H), 4.44 (d, J = 12.4 Hz, 1H), 4.07-3.87 (m, 2H), |
| | | | 3.57 (td, J = 13.9, 4.0 Hz, 2H), 2.45 (s, 3H); |
| | | | ¹⁹F NMR (376 MHz, CDCl₃) δ −57.61, −124.96 (d, J = 20.8 |
| | | | Hz), −130.64 |
| J772 | | ESIMS m/z 616 | ¹H NMR (400 MHz, CDCl₃) δ 8.55 (t, J = 8.3 Hz, 1H), 8.26 (d, J = |
| | | ([M + H]⁺) | 8.9 Hz, 2H), 7.95 (d, J = 8.7 Hz, 1H), 7.86 (d, J = 12.3 Hz, |
| | | | 1H), 7.57 (s, 1H), 7.40 (t, J = 7.3 Hz, 3H), 7.32 (d, J = 8.0 Hz, |
| | | | 1H), 7.00 (d, J = 7.1 Hz, 1H), 4.39 (d, J = 12.5 Hz, 1H), 4.29 (d, |
| | | | J = 12.5 Hz, 1H), 3.97 (d, J = 4.2 Hz, 2H), 3.27 (s, 3H), 2.44 (s, |
| | | | 3H); |
| | | | ¹⁹F NMR (376 MHz, CDCl₃) δ −57.61, −130.53 |
| J773 | | ESIMS m/z 643 | ¹H NMR (400 MHz, CDCl₃) δ 8.56 (t, J = 8.4 Hz, 1H), 8.26 (d, J = |
| | | ([M − H]⁻) | 8.8 Hz, 2H), 7.95 (d, J = 8.6 Hz, 1H), 7.86 (d, J = 11.2 Hz, |
| | | | 1H), 7.62 (s, 1H), 7.39 (d, J = 8.4 Hz, 2H), 7.32 (d, J = 8.5 Hz, |
| | | | 1H), 6.80 (dd, J = 8.6, 2.6 Hz, 1H), 6.47 (d, J = 2.7 Hz, 1H), |
| | | | 4.34 (d, J = 12.2 Hz, 1H), 4.20 (d, J = 12.3 Hz, 1H), 4.04-3.89 |
| | | | (m, 2H), 3.22 (s, 3H), 3.01 (s, 6H); |
| | | | ¹⁹F NMR (376 MHz, CDCl₃) δ −57.61, −130.35 |
| J774 | | | ¹H NMR (400 MHz, CDCl₃) δ 8.51 (s, 1H), 7.91-7.70 (m, 6H), |
| | | | 7.44-7.34 (m, 3H), 7.31 (s, 1H), 7.05 (s, 1H), 6.94-6.72 (m, |
| | | | 1H), 4.67-4.41 (m, 4H), 4.07-3.89 (m, 3H), 2.41 (s, 3H); |
| | | | ¹³CNMR(101 MHz, CDCl₃) δ 170.74, 168.51, 157.80, 145.09, |
| | | | 139.13, 133.17, 126.52, 126.15, 125.69, 124.29, 122.38, |
| | | | 121.75, 119.89, 116.02, 77.22, 59.49, 23.17 |
| J778 | | ESIMS m/z 743 | ¹H NMR (400 MHz, CDCl₃) δ 8.55 (s, 1H), 8.48 (t, J = 8.4 Hz, |
| | | ([M + H]⁺) | 1H), 7.98 (d, J = 8.7 Hz, 1H), 7.89 (dd, J = 12.1, 1.8 Hz, 1H), |
| | | | 7.82-7.74 (m, 2H), 7.50 (s, 1H), 7.45-7.33 (m, 4H), 7.03 (s, |
| | | | 1H), 4.69-4.43 (m, 2H), 4.24-4.08 (m, 2H), 4.04-3.89 (m, |
| | | | 2H), 3.67 (qd, J = 8.7, 4.6 Hz, 2H), 2.71 (t, J = 6.9 Hz, 1H), |
| | | | 2.45 (s, 3H), 2.38 (dd, J = 8.8, 4.4 Hz, 1H) |
| J779 | | HRMS-ESI (m/z) | ¹H NMR (400 MHz, CDCl₃) δ 8.55 (s, 1H), 8.47 (t, J = 8.4 Hz, |
| | | [M + H]⁺ calcd for | 1H), 7.98 (d, J = 8.6 Hz, 1H), 7.90 (dd, J = 12.1, 1.8 Hz, 1H), |
| | | C₂₉H₂₄F₄N₆O₃S, | 7.75-7.70 (m, 2H), 7.53 (t, J = 7.8 Hz, 2H), 7.48 (s, 1H), 7.41 |
| | | 613.1639; found, | (dd, J = 7.7, 4.8 Hz, 2H), 7.33 (d, J = 7.7 Hz, 1H), 7.02 (s, 1H), |
| | | 613.1651 | 4.47 (d, J = 12.7 Hz, 1H), 4.35 (d, J = 12.6 Hz, 1H), 3.95 (s, |
| | | | 2H), 3.55 (t, J = 6.7 Hz, 2H), 2.44 (s, 3H), 2.41-2.26 (m, 2H); |
| | | | ¹⁹F NMR (376 MHz, CDCl₃) δ −64.67, −131.42 |
| J780 | | HRMS-ESI (m/z) | ¹H NMR (400 MHz, CDCl₃) δ 8.54 (s, 1H), 8.17 (d, J = 8.5 Hz, |
| | | [M + H]⁺ calcd for | 1H), 8.05 (dd, J = 8.5, 2.0 Hz, 1H), 8.00 (d, J = 2.0 Hz, 1H), |
| | | C₂₉H₂₅F₃N₆O₃S, | 7.77-7.71 (m, 2H), 7.51 (td, J = 7.0, 6.2, 4.5 Hz, 2H), 7.45- |
| | | 595.1734; found, | 7.37 (m, 2H), 7.33 (d, J = 7.8 Hz, 1H), 7.08-7.01 (m, 2H), |
| | | 595.1749 | 4.63 (d, J = 12.4 Hz, 1H), 4.51 (d, J = 12.4 Hz, 1H), 3.94 (d, J = |
| | | | 2.1 Hz, 2H), 3.84-3.67 (m, 2H), 2.44 (s, 3H), 2.25 (d, J = 3.7 |
| | | | Hz, 3H); |
| | | | ¹⁹F NMR (376 MHz, CDCl₃) δ −73.75 |
| J781 | | HRMS-ESI (m/z) | ¹H NMR (400 MHz, CDCl₃) δ 8.55 (s, 1H), 8.18 (d, J = 8.5 Hz, |
| | | [M + H]⁺ calcd for | 1H), 8.05 (d, J = 8.5 Hz, 1H), 8.00 (s, 1H), 7.78-7.70 (m, 2H), |
| | | C₃₀H₂₇F₃N₆O₃S, | 7.52 (dd, J = 8.5, 7.2 Hz, 2H), 7.45-7.37 (m, 2H), 7.32 (d, J = |
| | | 609.189; found, | 7.8 Hz, 1H), 7.07-7.02 (m, 2H), 4.48 (d, J = 12.7 Hz, 1H), |
| | | 609.1897 | 4.36 (d, J = 12.6 Hz, 1H), 3.93 (s, 2H), 3.57 (td, J = 6.6, 1.7 Hz, |
| | | | 2H), 2.44 (s, 3H), 2.41-2.31 (m, 2H), 2.26 (s, 3H); |
| | | | ¹⁹F NMR (376 MHz, CDCl₃) δ −64.62 |
| J782 | | HRMS-ESI (m/z) | ¹H NMR (400 MHz, CDCl₃) δ 8.49 (s, 1H), 8.46 (d, J = 8.3 Hz, |
| | | [M + H]⁺ calcd for | 1H), 8.00-7.93 (m, 1H), 7.88 (dd, J = 12.1, 1.9 Hz, 1H), 7.74- |
| | | C₂₈H₂₁F₅N₆O₃S, | 7.66 (m, 2H), 7.47 (d, J = 3.0 Hz, 1H), 7.42 (d, J = 7.8 Hz, |
| | | 617.1389; found, | 1H), 7.37-7.32 (m, 1H), 7.25-7.19 (m, 2H), 7.04 (d, J = 1.6 |
| | | 617.1398 | Hz, 1H), 4.63 (d, J = 12.4 Hz, 1H), 4.50 (d, J = 12.4 Hz, 1H), |
| | | | 3.96 (d, J = 2.7 Hz, 2H), 3.71 (qd, J = 8.7, 4.3 Hz, 2H), 2.45 (s, |
| | | | 3H); |
| | | | ¹⁹F NMR (376 MHz, CDCl₃) δ −73.78, −112.90, −131.43 |
| J783 | | HRMS-ESI (m/z) | ¹H NMR (400 MHz, CDCl₃) δ 8.58 (s, 1H), 8.49 (t, J = 8.4 Hz, |
| | | [M + H]⁺ calcd for | 1H), 7.98 (d, J = 8.4 Hz, 1H), 7.93-7.86 (m, 1H), 7.86-7.78 |
| | | C₃₁H₂₇F₄N₇O₄S, | (m, 2H), 7.43 (d, J = 8.7 Hz, 2H), 7.31 (d, J = 7.9 Hz, 2H), 6.93 |
| | | 670.1854; found, | (s, 1H), 3.99 (d, J = 2.5 Hz, 2H), 3.81 (s, 3H), 2.46-2.29 (m, |
| | | 670.1853 | 3H), 1.59 (q, J = 7.5 Hz, 2H), 1.48 (s, 3H), 0.93 (t, J = 7.3 Hz, |
| | | | 3H); |
| | | | ¹⁹F NMR (376 MHz, CDCl₃) δ −54.60, −131.04 |
| J784 | | | ¹H NMR (400 MHz, CDCl₃) δ 8.58 (s, 1H), 8.20 (d, J = 8.5 Hz, |
| | | | 1H), 8.04 (d, J = 8.6 Hz, 1H), 7.99 (s, 1H), 7.83 (dd, J = 8.9, |
| | | | 2.3 Hz, 2H), 7.46-7.39 (m, 2H), 7.30 (d, J = 7.9 Hz, 2H), 7.09 |
| | | | (s, 1H), 6.94 (s, 1H), 3.97 (d, J = 1.8 Hz, 2H), 3.81 (d, J = 0.8 |
| | | | Hz, 4H), 2.39 (s, 4H), 2.26 (d, J = 6.1 Hz, 3H), 1.61 (p, J = 7.5 |
| | | | Hz, 2H), 0.93 (t, J = 7.3 Hz, 3H); |
| | | | ¹⁹F NMR (376 MHz, CDCl₃) δ −54.63 |
| J785 | | | ¹H NMR (500 MHz, CDCl₃) δ 8.40 (d, J = 3.0 Hz, 1H), 8.32 (q, |
| | | | J = 9.3, 8.3 Hz, 1H), 7.82 (t, J = 10.0 Hz, 1H), 7.76 (ddd, J = |
| | | | 12.0, 7.3, 1.7 Hz, 1H), 7.66-7.60 (m, 2H), 7.45 (dd, J = 15.3, |
| | | | 2.6 Hz, 1H), 7.25-7.15 (m, 7H), 7.11-7.08 (m, 2H), 6.95 (s, |
| | | | 1H), 3.66-3.57 (m, 1H), 3.41 (d, J = 18.0 Hz, 1H), 2.33 (s, |
| | | | 3H); |
| | | | ¹⁹F NMR (471 MHz, CDCl₃) δ −57.86, −130.92 |
| J786 | | ESIMS m/z 698 | ¹H NMR (400 MHz, CDCl₃) δ 8.56 (d, J = 8.9 Hz, 1H), 7.87- |
| | | ([M + H]⁺) | 7.79 (m, 4H), 7.61 (s, 1H), 7.49 (d, J = 1.4 Hz, 1H), 7.47-7.40 |
| | | | (m, 2H), 7.36 (dd, J = 7.1, 4.7 Hz, 2H), 7.24 (s, 1H), 7.05 (s, |
| | | | 1H), 4.63 (d, J = 12.4 Hz, 1H), 4.51 (d, J = 12.5 Hz, 1H), 3.97 |
| | | | (d, J = 2.2 Hz, 2H), 3.78-3.61 (m, 2H), 2.45 (s, 3H); |
| | | | ¹⁹F NMR (376 MHz, CDCl₃) δ −57.81, −73.75 |
| J787 | | ESIMS m/z 680 | ¹H NMR (400 MHz, CDCl₃) δ 8.56 (d, J = 8.9 Hz, 1H), 7.87- |
| | | ([M + H]⁺) | 7.80 (m, 4H), 7.62 (s, 1H), 7.49 (d, J = 1.4 Hz, 1H), 7.44 (dd, J = |
| | | | 4.1, 1.9 Hz, 1H), 7.40 (d, J = 7.8 Hz, 1H), 7.37-7.31 (m, |
| | | | 2H), 7.24 (s, 1H), 7.04 (s, 1H), 5.96-5.62 (m, 1H), 4.57 (d, J = |
| | | | 12.4 Hz, 1H), 4.45 (d, J = 12.3 Hz, 1H), 3.97 (d, J = 5.4 Hz, |
| | | | 2H), 3.57 (td, J = 13.9, 4.0 Hz, 2H), 2.45 (s, 3H); |
| | | | ¹⁹F NMR (376 MHz, CDCl₃) δ −57.81, −124.92 (d, J = 16.3 Hz) |
| J788 | | ESIMS m/z 630 | ¹H NMR (400 MHz, CDCl₃) δ 8.57 (d, J = 8.9 Hz, 1H), 7.88- |
| | | ([M + H]⁺) | 7.80 (m, 4H), 7.64 (s, 1H), 7.49 (d, J = 1.4 Hz, 1H), 7.46-7.38 |
| | | | (m, 2H), 7.33 (dd, J = 13.1, 7.7 Hz, 2H), 7.24 (s, 1H), 7.02 (s, |
| | | | 1H), 4.40 (d, J = 12.5 Hz, 1H), 4.30 (d, J = 12.6 Hz, 1H), 3.97 |
| | | | (d, J = 3.5 Hz, 2H), 3.27 (d, J = 2.8 Hz, 3H), 2.44 (s, 3H); |
| | | | ¹⁹F NMR (376 MHz, CDCl₃) δ −57.81 |
| J789 | | ESIMS m/z 659 | ¹H NMR (400 MHz, CDCl₃) δ 8.57 (d, J = 8.9 Hz, 1H), 7.86- |
| | | ([M + H]⁺) | 7.79 (m, 4H), 7.67 (s, 1H), 7.49 (s, 1H), 7.44 (d, J = 2.9 Hz, |
| | | | 1H), 7.36 (d, J = 2.5 Hz, 1H), 7.32 (d, J = 8.7 Hz, 1H), 7.24 (s, |
| | | | 1H), 6.79 (dd, J = 8.6, 2.6 Hz, 1H), 6.48 (d, J = 2.7 Hz, 1H), |
| | | | 4.35 (d, J = 12.2 Hz, 1H), 4.21 (d, J = 12.3 Hz, 1H), 3.96 (d, J = |
| | | | 5.6 Hz, 2H), 3.22 (s, 3H), 3.01 (s, 6H); |
| | | | ¹⁹F NMR (376 MHz, CDCl₃) δ −57.81 |
| J790 | | ESIMS m/z 698 | ¹H NMR (400 MHz, CDCl₃) δ 8.58 (d, J = 8.9 Hz, 1H), 7.88- |
| | | ([M + H]⁺) | 7.79 (m, 4H), 7.65 (s, 1H), 7.50 (d, J = 1.4 Hz, 1H), 7.44 (dd, J = |
| | | | 3.6, 1.9 Hz, 1H), 7.38-7.33 (m, 1H), 7.29 (d, J = 7.9 Hz, |
| | | | 1H), 7.24 (s, 1H), 7.04 (d, J = 2.0 Hz, 1H), 6.97 (d, J = 8.5 Hz, |
| | | | 1H), 4.26-4.19 (m, 2H), 4.06-3.88 (m, 2H), 2.55 (dtd, J = |
| | | | 16.4, 10.5, 6.1 Hz, 2H), 2.38 (s, 3H); |
| | | | ¹⁹F NMR (376 MHz, CDCl₃) δ −57.81, −64.56 |
| J791 | | ESIMS m/z 698 | ¹H NMR (400 MHz, CDCl₃) δ 8.55 (d, J = 8.9 Hz, 1H), 7.88- |
| | | ([M + H]⁺) | 7.80 (m, 4H), 7.68 (d, J = 8.0 Hz, 1H), 7.56 (s, 1H), 7.49 (d, J = |
| | | | 1.4 Hz, 1H), 7.44 (d, J = 2.5 Hz, 2H), 7.36 (d, J = 2.5 Hz, 1H), |
| | | | 7.24 (s, 1H), 7.02 (s, 1H), 4.49 (t, J = 6.1 Hz, 1H), 4.01 (d, J = |
| | | | 6.0 Hz, 2H), 3.45 (s, 3H), 2.46 (s, 3H); |
| | | | ¹⁹F NMR (376 MHz, CDCl₃) δ −57.81, −76.50 |
| J792 | | ESIMS m/z 628 | ¹H NMR (400 MHz, CDCl₃) δ 8.54 (d, J = 8.9 Hz, 1H), 7.87- |
| | | ([M + H]⁺) | 7.80 (m, 4H), 7.65 (s, 1H), 7.49 (s, 1H), 7.44 (dd, J = 4.4, 1.9 |
| | | | Hz, 1H), 7.40-7.31 (m, 3H), 7.24 (s, 1H), 6.90 (s, 1H), 4.00 (s, |
| | | | 2H), 2.72-2.59 (m, 1H), 2.39 (s, 3H), 1.20 (dd, J = 18.6, 6.8 |
| | | | Hz, 6H); |
| | | | ¹⁹F NMR (376 MHz, CDCl₃) δ −57.81 |
| J793 | | ESIMS m/z 717 | ¹H NMR (400 MHz, CDCl₃) δ 8.64 (s, 1H), 8.49 (t, J = 8.4 Hz, |
| | | ([M + H]⁺) | 1H), 7.98 (d, J = 8.7 Hz, 1H), 7.95-7.86 (m, 3H), 7.77 (d, J = |
| | | | 8.5 Hz, 2H), 7.49 (d, J = 3.1 Hz, 1H), 7.44-7.28 (m, 3H), 7.04 |
| | | | (s, 1H), 4.66-4.46 (m, 2H), 3.96 (d, J = 2.8 Hz, 2H), 3.72 (qq, |
| | | | J = 8.2, 3.7 Hz, 3H), 2.45 (s, 3H); |
| | | | ¹⁹F NMR (376 MHz, CDCl₃) δ −73.78, −84.69, −114.89, −131.29 |
| J794 | | ESIMS m/z 713 | ¹H NMR (400 MHz, CDCl₃) δ 8.64 (s, 1H), 8.20 (d, J = 8.5 Hz, |
| | | ([M + H]⁺) | 1H), 8.09-7.96 (m, 2H), 7.92 (d, J = 8.5 Hz, 2H), 7.77 (d, J = |
| | | | 8.5 Hz, 2H), 7.46-7.32 (m, 2H), 7.06 (s, 2H), 4.68-4.46 (m, |
| | | | 2H), 3.94 (d, J = 2.1 Hz, 2H), 3.73 (q, J = 8.7 Hz, 2H), 2.45 (s, |
| | | | 3H), 2.27 (s, 3H) |
| J795 | | ESIMS m/z 663 | ¹H NMR (400 MHz, CDCl₃) δ 8.64 (s, 1H), 8.50 (t, J = 8.4 Hz, |
| | | ([M + H]⁺) | 1H), 7.98 (d, J = 8.6 Hz, 1H), 7.91 (t, J = 8.7 Hz, 3H), 7.77 (d, J = |
| | | | 8.6 Hz, 2H), 7.40 (d, J = 7.8 Hz, 1H), 7.31 (d, J = 7.8 Hz, 1H), |
| | | | 7.02 (s, 1H), 4.48-4.32 (m, 2H), 3.96 (s, 2H), 3.41 (q, J = 7.0 |
| | | | Hz, 2H), 2.43 (s, 3H), 1.15 (t, J = 7.0 Hz, 3H); |
| | | | ¹⁹F NMR (376 MHz, CDCl₃) δ −84.69, −114.88, −131.07 |
| J796 | | ESIMS m/z 659 | ¹H NMR (400 MHz, CDCl₃) δ 8.64 (s, 1H), 8.22 (d, J = 8.6 Hz, |
| | | ([M + H]⁺) | 1H), 8.09-7.96 (m, 2H), 7.92 (d, J = 8.5 Hz, 2H), 7.76 (d, J = |
| | | | 8.5 Hz, 2H), 7.41 (t, J = 6.5 Hz, 1H), 7.30 (d, J = 8.2 Hz, 1H), |
| | | | 7.10 (s, 1H), 7.02 (d, J = 11.4 Hz, 1H), 4.49-4.28 (m, 2H), |
| | | | 3.94 (s, 2H), 3.46-3.33 (m, 2H), 2.43 (s, 3H), 2.27 (s, 3H), |
| | | | 1.16 (t, J = 7.0 Hz, 3H); |
| | | | ¹⁹F NMR (376 MHz, CDCl₃) δ −84.70, −114.87 |
| J797 | | | ¹H NMR (400 MHz, CDCl₃) δ 8.59-8.42 (m, 2H), 7.97 (d, J = |
| | | | 8.7 Hz, 1H), 7.88 (dd, J = 12.0, 1.8 Hz, 1H), 7.82-7.74 (m, |
| | | | 2H), 7.67-7.59 (m, 1H), 7.38 (d, J = 8.5 Hz, 2H), 7.14 (s, 1H), |
| | | | 6.80 (s, 1H), 3.99 (d, J = 1.6 Hz, 2H), 2.35 (d, J = 4.7 Hz, 6H), |
| | | | 2.01 (s, 3H); |
| | | | ¹³CNMR(101 MHz, CDCl₃) δ 171.43, 169.82, 161.44, 158.73, |
| | | | 152.42, 150.00, 147.60, 140.76, 137.50, 135.55, 134.61, |
| | | | 132.83, 131.66, 130.22, 127.07, 126.97, 125.38, 125.11, |
| | | | 122.07, 121.59, 120.37, 119.44, 118.26, 112.28, 112.07, |
| | | | 32.20, 20.01, 19.54, 13.00; |
| | | | ¹⁹F NMR (376 MHz, CDCl₃) δ −56.12, −130.91 |
| J798 | | | ¹H NMR (400 MHz, CDCl₃) δ 8.54 (s, 1H), 8.48 (t, J = 8.4 Hz, |
| | | | 1H), 7.96 (d, J = 9.0 Hz, 1H), 7.88 (dd, J = 12.0, 1.7 Hz, 1H), |
| | | | 7.81-7.74 (m, 2H), 7.59 (d, J = 2.5 Hz, 1H), 7.37 (t, J = 8.9 |
| | | | Hz, 3H), 7.19 (t, J = 7.7 Hz, 1H), 7.07-7.00 (m, 1H), 4.05- |
| | | | 3.93 (m, 2H), 3.73 (s, 3H), 2.40 (s, 3H); |
| | | | ¹³CNMR(101 MHz, CDCl₃) δ 171.40, 170.40, 161.53, 158.67, |
| | | | 153.63, 152.47, 150.05, 147.68, 140.84, 134.69, 132.54, |
| | | | 132.10, 127.38, 127.15, 127.05, 126.24, 125.45, 125.37, |
| | | | 123.79, 122.15, 121.67, 120.91, 120.45, 119.47, 118.35, |
| | | | 112.35, 112.13, 60.21, 32.27, 15.64; |
| | | | ¹⁹F NMR (376 MHz, CDCl₃) δ −57.96, −131.06 |
| J799 | | | ¹H NMR (400 MHz, CDCl₃) δ 8.54 (s, 1H), 8.48 (t, J = 8.4 Hz, |
| | | | 1H), 7.97 (d, J = 8.3 Hz, 1H), 7.88 (dd, J = 12.0, 1.6 Hz, 1H), |
| | | | 7.81-7.76 (m, 2H), 7.76-7.72 (m, 1H), 7.51 (d, J = 2.4 Hz, |
| | | | 1H), 7.47 (d, J = 8.1 Hz, 1H), 7.38 (d, J = 8.5 Hz, 2H), 7.12 (s, |
| | | | 1H), 3.98 (q, J = 18.1 Hz, 2H), 2.51 (s, 3H); |
| | | | ¹³C NMR (101 MHz, CDCl₃) δ 171.61, 170.25, 162.04, 158.88, |
| | | | 152.99, 150.57, 148.23, 144.24, 141.38, 135.22, 132.17, |
| | | | 131.05, 130.81, 127.49, 126.06, 125.98, 125.39, 125.08, |
| | | | 122.69, 122.21, 121.45, 120.99, 119.99, 112.87, 112.66, |
| | | | 32.77, 21.12; |
| | | | ¹⁹F NMR (376 MHz, CDCl₃) δ −57.79, −61.17, −131.52 |
| J800 | | ESIMS m/z 568 | ¹H NMR (300 MHz, DMSO-d₆) δ 9.96 (s, 1H), 9.36 (s, 1H), 8.45 |
| | | ([M + H]⁺) | (d, J = 1.8 Hz, 1H), 8.26 (s, 1H), 8.06 (d, J = 9.0 Hz, 2H), 7.99 |
| | | | (d, J = 8.8 Hz, 2H), 7.79 (d, J = 8.7 Hz, 2H), 7.62 (d, J = 8.5 |
| | | | Hz, 2H), 7.16-7.06 (m, 2H), 6.90 (s, 1H), 3.95 (s, 2H), 2.24 (s, |
| | | | 3H) |
| J801 | | ESIMS m/z 693 | ¹H NMR (400 MHz, CDCl₃) δ 8.54 (s, 1H), 8.20 (d, J = 8.5 Hz, |
| | | ([M + H]⁺) | 1H), 8.03 (d, J = 8.6 Hz, 1H), 7.98 (s, 1H), 7.85-7.77 (m, 2H), |
| | | | 7.42-7.37 (m, 3H), 7.31 (d, J = 7.6 Hz, 1H), 7.11 (s, 1H), 7.04 |
| | | | (s, 1H), 4.62-4.50 (m, 2H), 4.44 (d, J = 12.4 Hz, 2H), 3.99 (d, |
| | | | J = 18.1 Hz, 1H), 3.90 (d, J = 18.0 Hz, 1H), 3.74-3.52 (m, |
| | | | 2H), 2.43 (s, 3H), 2.26 (s, 3H); |
| | | | ¹⁹F NMR (376 MHz, CDCl₃) δ −85.88, −87.85 |
| J802 | | ESIMS m/z 581 | ¹H NMR (400 MHz, CDCl₃) δ 8.08-8.02 (m, 2H), 8.00 (d, J = |
| | | ([M + H]⁺) | 8.6 Hz, 2H), 7.61 (d, J = 8.7 Hz, 2H), 7.54-7.44 (m, 2H), 7.42 |
| | | | (d, J = 7.8 Hz, 1H), 7.39-7.30 (m, 2H), 7.04 (s, 1H), 4.62 (d, J = |
| | | | 12.3 Hz, 1H), 4.50 (d, J = 12.3 Hz, 1H), 3.96 (d, J = 2.1 Hz, |
| | | | 2H), 3.73 (qd, J = 8.6, 1.7 Hz, 1H), 2.43 (s, 3H); |
| | | | ¹⁹F NMR (376 MHz, CDCl₃) δ −73.75 |

### Example A: Bioassays on Beet Armyworm (Spodoptera exigua) (“BAW”) and Cabbage Looper (Trichoplusia ni) (“CL”)

BAW has few effective parasites, diseases, or predators to lower its population. BAW infests many weeds, trees, grasses, legumes, and field crops. In various places, it is of economic concern upon asparagus, cotton, corn, soybeans, tobacco, alfalfa, sugar beets, peppers, tomatoes, potatoes, onions, peas, sunflowers, and citrus, among other plants. CL is a member of the moth family Noctuidae. It is found throughout the world. It attacks cabbage, cauliflower, broccoli, Brussel sprouts, tomatoes, cucumbers, potatoes, kale, turnips, mustard, peppers, eggplant, watermelons, melons, squash, cantaloupe, peas, beans, collards, lettuce, spinach, celery, parsley, beets, peas, alfalfa, soybeans, and cotton. This species is very destructive to plants due to its voracious consumption of leaves. In the case of cabbage, however, they feed not only on the wrapper leaves, but also may bore into the developing head. The larvae consume three times their weight in plant material daily. The feeding sites are marked by large accumulations of sticky, wet fecal material.

Consequently, because of the above factors, control of these pests is important. Furthermore, molecules that control these pests (BAW and CL), which are known as chewing pests, are useful in controlling other pests that chew on plants.

Certain molecules disclosed in this document were tested against BAW and CL using procedures described in the following examples. In the reporting of the results, the “BAW & CL Rating Table” was used (See Table Section).

### Bioassays on BAW

Bioassays on BAW were conducted using a 128-well diet tray assay. one to five second instar BAW larvae were placed in each well (3 mL) of the diet tray that had been previously filled with 1 mL of artificial diet to which 50 μg/cm² of the test compound (dissolved in 50 μL of 90:10 acetone-water mixture) had been applied (to each of eight wells) and then allowed to dry. Trays were covered with a clear self-adhesive cover and held at 25° C., 14:10 light-dark cycle for five to seven days. Percent mortality was recorded for the larvae in each well; activity in the eight wells was then averaged. The results are indicated in the table entitled “Table ABC: Biological Results” (See Table Section).

### Bioassays on CL

Bioassays on CL were conducted using a 128-well diet tray assay. one to five second instar CL larvae were placed in each well (3 mL) of the diet tray that had been previously filled with 1 mL of artificial diet to which 50 μg/cm² of the test compound (dissolved in 50 μL of 90:10 acetone-water mixture) had been applied (to each of eight wells) and then allowed to dry. Trays were covered with a clear self-adhesive cover and held at 25° C., 14:10 light-dark cycle for five to seven days. Percent mortality was recorded for the larvae in each well; activity in the eight wells was then averaged. The results are indicated in the table entitled “Table ABC: Biological Results” (See Table Section).

### Example B: Bioassays on Green Peach Aphid (“GPA”) (Myzus Persicae)

GPA is the most significant aphid pest of peach trees, causing decreased growth, shriveling of the leaves, and the death of various tissues. It is also hazardous because it acts as a vector for the transport of plant viruses, such as potato virus Y and potato leafroll virus to members of the nightshade/potato family Solanaceae, and various mosaic viruses to many other food crops. GPA attacks such plants as broccoli, burdock, cabbage, carrot, cauliflower, daikon, eggplant, green beans, lettuce, macadamia, papaya, peppers, sweet potatoes, tomatoes, watercress, and zucchini, among other plants. GPA also attacks many ornamental crops such as carnation, chrysanthemum, flowering white cabbage, poinsettia, and roses. GPA has developed resistance to many pesticides. Consequently, because of the above factors control of this pest is important. Furthermore, molecules that control this pest (GPA), which is known as a sucking pest, are useful in controlling other pests that suck on plants.

Certain molecules disclosed in this document were tested against GPA using procedures described in the following example. In the reporting of the results, the “GPA Rating Table” was used (See Table Section).

Cabbage seedlings grown in 3-inch pots, with 2-3 small (3-5 cm) true leaves, were used as test substrate. The seedlings were infested with 20-50 GPA (wingless adult and nymph stages) one day prior to chemical application. Four pots with individual seedlings were used for each treatment. Test compounds (2 mg) were dissolved in 2 mL of acetone/methanol (1:1) solvent, forming stock solutions of 1000 ppm test compound. The stock solutions were diluted 5× with 0.025% Tween 20 in water to obtain the solution at 200 ppm test compound. A hand-held aspirator-type sprayer was used for spraying a solution to both sides of cabbage leaves until runoff. Reference plants (solvent check) were sprayed with the diluent only containing 20% by volume of acetone/methanol (1:1) solvent. Treated plants were held in a holding room for three days at approximately 25° C. and ambient relative humidity (RH) prior to grading. Evaluation was conducted by counting the number of live aphids per plant under a microscope. Percent Control was measured by using Abbott's correction formula (W. S. Abbott, “A Method of Computing the Effectiveness of an Insecticide” J. Econ. Entomol. 18 (1925), pp. 265-267) as follows.

Corrected % Control=100*(X−Y)/X
where
X=No. of live aphids on solvent check plants and
Y=No. of live aphids on treated plants

The results are indicated in the table entitled “Table ABC: Biological Results” (See Table Section).

### Example C: Bioassays on Yellow Fever Mosquito “YFM” (Aedes aegypti)

YFM prefers to feed on humans during the daytime and is most frequently found in or near human habitations. YFM is a vector for transmitting several diseases. It is a mosquito that can spread the dengue fever and yellow fever viruses. Yellow fever is the second most dangerous mosquito-borne disease after malaria. Yellow fever is an acute viral hemorrhagic disease, and up to 50% of severely affected persons without treatment will die from yellow fever. There are an estimated 200,000 cases of yellow fever, causing 30,000 deaths, worldwide each year. Dengue fever is a nasty, viral disease; it is sometimes called “breakbone fever” or “break-heart fever” because of the intense pain it can produce. Dengue fever kills about 20,000 people annually. Consequently, because of the above factors control of this pest is important. Furthermore, molecules that control this pest (YFM), which is known as a sucking pest, are useful in controlling other pests that cause human and animal suffering.

Certain molecules disclosed in this document were tested against YFM using procedures described in the following paragraph. In the reporting of the results, the “YFM Rating Table” was used (See Table Section).

Master plates containing 400 μg of a molecule dissolved in 100 μL of dimethyl sulfoxide (DMSO) (equivalent to a 4000 ppm solution) are used. A master plate of assembled molecules contains 15 μL per well. To this plate, 135 μL of a 90:10 water:acetone mixture is added to each well. A robot (Biomek® NXP Laboratory Automation Workstation) is programmed to dispense 15 μL aspirations from the master plate into an empty 96-well shallow plate (“daughter” plate). There are 6 reps (“daughter” plates) created per master. The created daughter plates are then immediately infested with YFM larvae.

The day before plates are to be treated, mosquito eggs are placed in Millipore water containing liver powder to begin hatching (4 g. into 400 mL). After the daughter plates are created using the robot, they are infested with 220 μL of the liver powder/larval mosquito mixture (about 1 day-old larvae). After plates are infested with mosquito larvae, a non-evaporative lid is used to cover the plate to reduce drying. Plates are held at room temperature for 3 days prior to grading. After 3 days, each well is observed and scored based on mortality.

### Table Section

BAW & CL Rating Table

| % Control (or Mortality) | Rating |
|---|---|
| 50-100 | A |
| More than 0-Less than 50 | B |
| Not Tested | C |
| No activity noticed in this bioassay | D |

GPA & YFM Rating Table

| % Control (or Mortality) | Rating |
|---|---|
| 80-100 | A |
| More than 0-Less than 80 | B |
| Not Tested | C |
| No activity noticed in this bioassay | D |

TABLE ABC
Biological Results

Species

| Cmpd ID | BAW | CL | GPA | YFM |
|---|---|---|---|---|
| H1 | A | A | B | A |
| H2 | A | A | A | C |
| H3 | A | A | A | A |
| H4 | A | A | C | C |
| H6 | A | A | C | C |
| H7 | A | A | B | C |
| H8 | A | A | C | C |
| H10 | A | A | C | C |
| H11 | A | A | B | A |
| H12 | A | A | C | C |
| H13 | A | A | C | C |
| H14 | A | A | B | C |
| H15 | A | A | C | C |
| H16 | A | A | C | C |
| H17 | A | A | C | C |
| H20 | A | A | B | C |
| H21 | A | A | B | A |
| C1 | A | A | C | C |
| C2 | A | A | B | C |
| C3 | A | A | C | C |
| J1 | A | A | B | C |
| J2 | A | A | C | C |
| J3 | C | C | C | C |
| J4 | A | A | C | C |
| J5 | A | A | C | C |
| J6 | A | A | C | C |
| J7 | A | A | C | C |
| J8 | A | A | C | C |
| J9 | C | C | C | C |
| J10 | A | A | C | C |
| J11 | A | A | C | C |
| J12 | A | A | C | C |
| J13 | A | A | C | C |
| J14 | A | A | C | C |
| J15 | A | A | C | C |
| J16 | C | C | C | C |
| J18 | A | A | C | C |
| J19 | A | A | B | C |
| J20 | C | C | C | C |
| J21 | A | A | C | C |
| J22 | A | A | C | C |
| J23 | A | A | C | C |
| J24 | A | A | C | C |
| J25 | A | A | C | C |
| J26 | A | A | C | C |
| J27 | A | A | C | C |
| J28 | A | A | C | C |
| J29 | A | A | C | C |
| J30 | A | A | C | C |
| J31 | A | A | C | C |
| J32 | A | A | B | C |
| J33 | C | C | C | C |
| J34 | A | A | C | C |
| J35 | A | A | B | C |
| J36 | A | A | C | C |
| J37 | A | A | D | C |
| J38 | A | A | C | C |
| J39 | A | A | C | C |
| J40 | A | A | C | C |
| J41 | C | C | B | C |
| J42 | A | A | C | C |
| J43 | A | A | C | C |
| J44 | A | A | C | C |
| J45 | A | A | C | C |
| J46 | A | A | C | C |
| J47 | A | A | C | C |
| J48 | A | A | C | C |
| J49 | A | A | C | C |
| J50 | A | A | C | C |
| J51 | A | A | C | C |
| J52 | C | C | C | C |
| J53 | A | A | C | C |
| J54 | A | A | C | C |
| J55 | A | A | C | C |
| J56 | A | A | C | C |
| J57 | A | A | C | C |
| J58 | A | A | C | C |
| J59 | A | A | C | C |
| J60 | A | A | C | C |
| J61 | A | A | C | C |
| J62 | A | A | C | C |
| J63 | A | A | C | C |
| J64 | A | A | C | C |
| J65 | A | A | C | C |
| J66 | A | A | B | C |
| J67 | A | A | C | C |
| J68 | A | A | D | C |
| J69 | A | A | C | C |
| J70 | A | A | B | C |
| J71 | A | A | B | C |
| J72 | A | A | B | C |
| J73 | A | A | B | C |
| J74 | A | A | B | C |
| J75 | A | A | B | C |
| J76 | C | C | B | C |
| J77 | A | A | B | C |
| J78 | A | A | C | C |
| J79 | A | A | C | C |
| J80 | A | A | C | C |
| J81 | A | A | C | C |
| J82 | A | A | C | C |
| J83 | A | A | C | C |
| J84 | A | A | C | C |
| J85 | A | A | C | C |
| J86 | A | A | B | C |
| J87 | A | A | B | C |
| J88 | C | C | C | C |
| J89 | A | A | C | C |
| J90 | A | A | C | C |
| J91 | A | A | C | C |
| J92 | A | A | C | C |
| J93 | A | A | C | C |
| J94 | A | A | C | C |
| J95 | A | A | C | C |
| J96 | A | A | C | C |
| J97 | A | A | C | C |
| J98 | A | A | C | C |
| J99 | A | A | C | C |
| J100 | A | A | C | C |
| J101 | A | A | C | C |
| J102 | A | A | C | C |
| J103 | A | A | B | C |
| J104 | A | A | B | C |
| J105 | A | A | B | C |
| J106 | A | A | C | C |
| J107 | A | A | C | C |
| J108 | A | A | C | C |
| J109 | A | A | C | C |
| J110 | A | A | C | C |
| J111 | A | A | C | C |
| J112 | A | A | C | C |
| J113 | A | A | B | C |
| J114 | A | A | C | C |
| J115 | A | A | C | C |
| J116 | A | A | B | C |
| J117 | A | A | C | C |
| J118 | A | A | C | C |
| J119 | A | A | B | C |
| J120 | A | A | B | C |
| J121 | B | A | C | C |
| J122 | A | A | B | C |
| J123 | A | A | B | C |
| J124 | A | A | C | C |
| J125 | A | A | C | C |
| J126 | A | A | B | C |
| J127 | A | A | B | C |
| J128 | A | A | C | C |
| J129 | A | A | C | C |
| J130 | A | A | C | C |
| J131 | A | A | D | C |
| J132 | A | A | B | C |
| J133 | A | A | B | C |
| J134 | A | A | B | C |
| J135 | A | A | B | C |
| J136 | A | A | B | C |
| J137 | A | A | B | C |
| J138 | A | A | B | C |
| J139 | A | A | B | C |
| J140 | A | A | C | C |
| J141 | A | A | B | C |
| J142 | A | A | B | C |
| J143 | A | A | B | C |
| J144 | A | A | C | C |
| J145 | A | A | B | C |
| J146 | A | A | B | C |
| J147 | A | A | D | C |
| J148 | A | A | B | C |
| J149 | A | A | B | C |
| J150 | A | A | C | C |
| J151 | A | A | B | C |
| J152 | A | A | B | C |
| J153 | A | A | B | C |
| J154 | A | A | B | C |
| J155 | A | A | D | C |
| J156 | C | C | C | C |
| J157 | A | A | C | C |
| J158 | A | A | C | C |
| J159 | A | A | C | C |
| J160 | A | A | C | C |
| J161 | A | B | C | C |
| J162 | A | A | D | C |
| J163 | A | A | C | C |
| J164 | A | A | D | C |
| J165 | A | A | D | C |
| J166 | A | A | C | C |
| J167 | A | A | D | C |
| J168 | A | A | B | C |
| J169 | A | A | B | C |
| J170 | A | A | C | C |
| J171 | A | A | C | C |
| J172 | A | A | B | C |
| J173 | A | A | D | C |
| J174 | A | A | C | C |
| J175 | A | A | C | C |
| J176 | B | D | C | C |
| J177 | C | C | C | C |
| J178 | A | A | D | C |
| J179 | A | A | C | C |
| J180 | A | A | B | C |
| J181 | A | A | B | C |
| J182 | A | A | C | C |
| J183 | A | A | B | C |
| J184 | A | A | C | C |
| J185 | C | C | C | C |
| J186 | A | A | C | C |
| J187 | A | A | C | C |
| J188 | A | A | D | C |
| J189 | A | A | B | C |
| J190 | C | C | C | C |
| J191 | C | C | B | C |
| J192 | A | A | B | C |
| J193 | A | A | B | C |
| J194 | A | A | B | C |
| J195 | A | A | C | C |
| J196 | A | A | B | C |
| J197 | C | C | C | C |
| J198 | A | A | D | C |
| J199 | A | A | C | C |
| J200 | A | A | C | C |
| J201 | C | C | C | C |
| J202 | C | C | C | C |
| J203 | C | C | C | C |
| J204 | A | A | C | C |
| J205 | A | A | C | C |
| J206 | A | A | C | C |
| J211 | A | A | C | C |
| J212 | A | A | C | C |
| J213 | A | A | C | C |
| J214 | A | A | C | C |
| J215 | A | A | C | C |
| J216 | C | C | C | C |
| J217 | A | A | C | C |
| J218 | A | A | C | C |
| J219 | A | A | C | C |
| J220 | A | A | C | C |
| J221 | A | A | C | C |
| J222 | A | A | C | C |
| J223 | A | A | B | C |
| J224 | A | A | B | C |
| J225 | A | A | B | C |
| J226 | A | A | C | C |
| J227 | A | A | D | C |
| J228 | A | A | C | C |
| J229 | A | A | B | C |
| J230 | A | A | B | C |
| J231 | A | A | B | C |
| J232 | A | A | B | C |
| J233 | A | A | D | C |
| J234 | C | C | C | C |
| J235 | C | C | D | C |
| J236 | A | A | C | C |
| J237 | A | A | C | C |
| J238 | A | A | C | C |
| J239 | A | A | C | C |
| J240 | A | A | C | C |
| J241 | A | A | C | C |
| J242 | A | A | D | C |
| J243 | A | A | C | C |
| J244 | A | A | C | C |
| J245 | A | A | C | C |
| J246 | A | A | C | C |
| J247 | A | A | C | C |
| J248 | A | A | C | C |
| J249 | A | A | C | C |
| J250 | A | A | C | C |
| J251 | A | A | C | C |
| J252 | A | A | C | C |
| J253 | A | A | C | C |
| J254 | A | A | C | C |
| J255 | A | A | C | C |
| J256 | C | C | C | C |
| J257 | A | A | C | C |
| J258 | A | A | C | C |
| J259 | A | A | C | C |
| J260 | A | A | C | C |
| J261 | A | A | C | C |
| J262 | A | A | C | C |
| J263 | A | A | C | C |
| J264 | A | A | C | C |
| J265 | A | A | C | C |
| J266 | A | A | C | C |
| J267 | A | A | C | C |
| J268 | A | A | C | C |
| J269 | A | A | C | C |
| J270 | A | A | C | C |
| J271 | A | A | C | C |
| J272 | A | A | C | C |
| J273 | A | A | C | C |
| J274 | A | A | C | C |
| J275 | A | A | C | C |
| J276 | A | A | C | C |
| J277 | A | A | C | C |
| J278 | A | A | C | C |
| J279 | A | A | C | C |
| J280 | A | A | C | C |
| J281 | A | A | C | C |
| J282 | A | A | C | C |
| J283 | A | A | C | C |
| J284 | A | A | C | C |
| J285 | A | A | C | C |
| J286 | A | A | C | C |
| J287 | A | A | B | C |
| J288 | A | A | B | C |
| J289 | A | A | B | C |
| J290 | A | A | D | C |
| J291 | A | A | B | C |
| J292 | A | A | C | C |
| J293 | A | A | C | C |
| J294 | A | A | C | C |
| J295 | A | A | C | C |
| J296 | A | A | C | C |
| J297 | A | A | C | C |
| J298 | A | A | C | C |
| J299 | A | A | C | C |
| J300 | A | A | C | C |
| J301 | A | A | C | C |
| J302 | A | A | C | C |
| J303 | A | A | C | C |
| J304 | A | A | C | C |
| J305 | A | A | C | C |
| J306 | A | A | C | C |
| J307 | A | A | C | C |
| J308 | A | A | C | C |
| J309 | A | A | C | C |
| J310 | A | A | C | C |
| J311 | A | A | C | C |
| J312 | A | A | B | C |
| J313 | A | A | C | B |
| J314 | A | A | B | C |
| J315 | A | A | B | C |
| J316 | A | A | B | C |
| J317 | A | A | C | C |
| J318 | A | A | B | C |
| J319 | A | A | B | C |
| J320 | A | A | C | C |
| J321 | A | A | C | C |
| J322 | A | A | C | C |
| J323 | A | A | C | C |
| J324 | A | A | B | C |
| J325 | A | A | B | C |
| J326 | A | A | B | C |
| J327 | A | A | B | C |
| J328 | A | A | B | C |
| J329 | A | A | C | C |
| J330 | C | C | C | C |
| J331 | A | A | D | C |
| J332 | A | A | C | C |
| J333 | A | A | C | C |
| J334 | A | A | C | C |
| J335 | C | C | C | C |
| J336 | A | A | C | C |
| J337 | A | A | C | C |
| J338 | A | A | C | C |
| J339 | A | A | C | C |
| J340 | A | A | D | C |
| J341 | A | A | C | C |
| J342 | A | A | C | C |
| J343 | A | A | C | C |
| J344 | A | A | B | C |
| J345 | A | A | C | C |
| J346 | A | A | C | C |
| J347 | A | A | C | C |
| J348 | A | A | C | C |
| J349 | A | A | C | C |
| J350 | A | A | B | C |
| J351 | A | A | C | C |
| J352 | A | A | C | C |
| J353 | A | A | C | C |
| J354 | A | A | C | C |
| J355 | A | A | C | C |
| J356 | C | C | C | C |
| J357 | A | A | C | C |
| J358 | A | A | C | C |
| J359 | A | A | C | C |
| J360 | A | A | C | C |
| J361 | A | A | D | C |
| J362 | A | A | B | C |
| J363 | A | A | B | C |
| J364 | A | A | B | C |
| J365 | A | A | B | C |
| J366 | A | A | B | C |
| J367 | A | A | D | C |
| J368 | A | A | B | C |
| J369 | A | A | C | C |
| J370 | A | A | C | C |
| J371 | A | A | C | C |
| J372 | A | A | C | C |
| J373 | A | A | C | C |
| J374 | A | A | C | C |
| J375 | A | A | C | C |
| J376 | A | A | C | C |
| J377 | A | A | C | C |
| J378 | A | A | C | C |
| J379 | A | A | C | C |
| J380 | A | A | C | C |
| J381 | A | A | C | C |
| J382 | A | A | C | C |
| J383 | A | A | C | C |
| J384 | A | A | C | C |
| J385 | A | A | C | C |
| J386 | A | A | C | C |
| J387 | A | A | C | C |
| J388 | A | A | C | C |
| J389 | A | A | C | C |
| J390 | A | A | C | C |
| J391 | A | A | C | C |
| J392 | A | A | C | C |
| J393 | A | A | C | C |
| J394 | A | A | C | C |
| J395 | A | A | C | C |
| J396 | A | A | C | C |
| J397 | A | A | C | C |
| J398 | A | A | C | C |
| J399 | A | A | C | C |
| J400 | A | A | C | C |
| J401 | A | A | C | C |
| J402 | A | A | C | C |
| J403 | A | A | C | C |
| J404 | A | A | C | C |
| J405 | A | A | C | C |
| J406 | A | A | C | C |
| J407 | A | A | C | C |
| J408 | A | A | C | C |
| J409 | A | A | C | C |
| J410 | A | A | C | C |
| J411 | C | C | C | C |
| J412 | C | C | C | C |
| J413 | A | A | C | C |
| J414 | A | A | C | C |
| J415 | D | D | C | C |
| J416 | D | A | C | C |
| J417 | C | C | C | C |
| J418 | B | D | C | C |
| J419 | C | C | C | C |
| J420 | A | A | C | C |
| J421 | A | A | C | C |
| J422 | A | A | C | C |
| J423 | A | A | C | C |
| J425 | A | A | C | C |
| J426 | A | A | C | C |
| J427 | A | A | C | C |
| J428 | A | A | C | C |
| J429 | A | A | C | C |
| J430 | A | A | C | C |
| J431 | A | A | C | C |
| J432 | A | A | C | C |
| J433 | A | A | C | C |
| J434 | A | A | C | C |
| J435 | A | A | C | C |
| J436 | A | A | C | C |
| J437 | A | A | C | C |
| J438 | A | A | C | C |
| J439 | C | C | C | C |
| J440 | A | B | C | C |
| J441 | A | A | C | C |
| J442 | A | A | C | C |
| J443 | A | A | C | C |
| J444 | A | A | C | C |
| J445 | A | A | C | C |
| J446 | A | A | C | C |
| J447 | A | A | C | C |
| J448 | A | A | C | C |
| J449 | A | A | C | C |
| J450 | A | A | C | C |
| J451 | A | A | C | C |
| J452 | A | A | C | C |
| J453 | A | A | C | C |
| J454 | A | A | C | C |
| J455 | A | A | C | C |
| J456 | A | A | C | C |
| J457 | A | A | C | C |
| J458 | A | A | C | C |
| J459 | A | A | C | C |
| J460 | A | A | C | C |
| J461 | A | A | C | C |
| J462 | C | C | C | C |
| J463 | A | A | C | C |
| J465 | A | A | C | C |
| J466 | A | A | C | C |
| J467 | A | A | C | C |
| J468 | A | A | C | C |
| J469 | A | A | C | C |
| J470 | C | C | C | C |
| J471 | C | C | C | C |
| J472 | C | C | C | C |
| J473 | A | A | C | C |
| J474 | A | A | C | C |
| J476 | A | A | C | C |
| J477 | A | A | C | C |
| J480 | C | C | C | C |
| J481 | A | A | C | C |
| J482 | A | A | C | C |
| J483 | A | A | C | C |
| J484 | A | A | C | C |
| J485 | A | A | C | C |
| J486 | A | A | C | C |
| J487 | A | A | C | C |
| J488 | A | A | C | C |
| J489 | A | A | C | C |
| J490 | C | C | C | C |
| J491 | A | D | C | C |
| J492 | D | D | C | C |
| J493 | A | A | C | C |
| J494 | A | A | C | C |
| J495 | B | D | C | C |
| J496 | A | A | C | C |
| J497 | A | A | C | C |
| J498 | A | A | C | C |
| J499 | A | A | C | C |
| J500 | A | A | C | C |
| J501 | A | D | C | C |
| J502 | D | D | C | C |
| J503 | D | A | C | C |
| J504 | B | A | C | C |
| J505 | D | A | C | C |
| J506 | A | A | C | C |
| J507 | A | A | C | C |
| J508 | A | A | C | C |
| J509 | A | A | C | C |
| J510 | A | A | B | A |
| J511 | A | A | C | C |
| J512 | A | A | C | C |
| J513 | A | A | C | C |
| J514 | A | A | C | C |
| J515 | A | A | C | C |
| J516 | A | A | C | C |
| J517 | A | A | C | C |
| J518 | A | A | C | C |
| J519 | A | A | C | C |
| J520 | A | A | C | C |
| J521 | A | A | C | C |
| J522 | A | A | C | C |
| J523 | A | A | C | C |
| J524 | A | A | C | C |
| J525 | A | A | C | C |
| J526 | A | A | C | C |
| J527 | A | A | C | C |
| J529 | A | A | C | C |
| J532 | C | C | C | C |
| J533 | C | C | C | C |
| J534 | A | A | C | C |
| J535 | A | A | C | C |
| J536 | C | C | C | C |
| J541 | A | A | C | C |
| J543 | A | A | C | C |
| J544 | A | A | C | C |
| J546 | A | A | C | C |
| J547 | A | A | C | C |
| J548 | A | A | C | C |
| J549 | A | A | C | C |
| J550 | A | A | C | C |
| J551 | A | A | C | C |
| J552 | A | A | C | C |
| J553 | A | A | C | C |
| J554 | A | A | C | C |
| J555 | A | A | C | C |
| J556 | A | A | C | C |
| J557 | A | A | C | C |
| J558 | A | A | C | C |
| J559 | A | A | C | C |
| J560 | A | A | C | C |
| J561 | A | A | C | C |
| J562 | A | A | C | C |
| J563 | A | A | C | C |
| J564 | A | A | C | C |
| J565 | A | A | C | C |
| J566 | A | A | C | C |
| J567 | A | A | C | C |
| J568 | A | A | D | C |
| J569 | A | A | C | C |
| J570 | A | A | B | C |
| J571 | A | A | C | C |
| J572 | A | A | D | A |
| J573 | C | C | C | C |
| J574 | A | A | B | C |
| J575 | A | A | C | C |
| J576 | A | A | C | C |
| J577 | A | A | C | C |
| J578 | A | A | C | C |
| J579 | A | A | C | C |
| J580 | A | A | C | C |
| J581 | A | A | C | C |
| J582 | A | A | C | C |
| J583 | A | A | C | C |
| J584 | A | A | C | C |
| J585 | A | A | C | C |
| J586 | A | A | C | C |
| J587 | A | A | C | C |
| J588 | A | A | C | C |
| J589 | A | A | C | C |
| J590 | A | A | C | C |
| J591 | A | A | C | C |
| J592 | A | A | C | C |
| J593 | C | C | C | C |
| J594 | A | A | C | C |
| J595 | A | A | C | C |
| J596 | A | A | C | C |
| J597 | A | A | C | C |
| J598 | A | A | C | C |
| J599 | A | A | C | C |
| J600 | A | A | C | C |
| J601 | A | A | C | C |
| J602 | A | A | C | C |
| J603 | A | A | C | C |
| J604 | A | A | C | C |
| J605 | A | A | C | C |
| J606 | A | A | C | C |
| J607 | A | A | C | C |
| J608 | A | A | C | C |
| J609 | A | A | C | C |
| J610 | A | A | C | C |
| J611 | A | A | C | C |
| J612 | A | A | C | C |
| J613 | A | A | C | C |
| J614 | A | A | C | C |
| J615 | A | A | C | C |
| J616 | A | A | C | C |
| J617 | A | A | C | C |
| J618 | A | A | C | C |
| J619 | A | A | C | C |
| J620 | A | A | C | C |
| J621 | A | A | C | C |
| J622 | A | A | C | C |
| J623 | A | A | C | C |
| J624 | C | C | C | C |
| J625 | A | A | C | C |
| J626 | A | A | C | C |
| J627 | A | A | C | C |
| J628 | A | A | C | C |
| J629 | A | A | C | C |
| J630 | A | A | C | C |
| J631 | A | A | C | C |
| J632 | A | A | C | C |
| J633 | A | A | C | C |
| J634 | A | A | C | C |
| J635 | A | A | D | C |
| J636 | A | B | D | C |
| J640 | A | A | C | C |
| J641 | A | A | C | C |
| J642 | A | A | C | C |
| J646 | A | A | B | C |
| J647 | A | A | B | C |
| J648 | A | A | B | C |
| J649 | A | A | B | C |
| J650 | A | A | B | C |
| J651 | A | A | D | C |
| J652 | A | A | B | C |
| J653 | A | A | B | A |
| J659 | A | A | B | C |
| J660 | A | A | C | C |
| J661 | A | A | C | C |
| J662 | A | A | C | C |
| J663 | A | A | B | C |
| J664 | A | A | C | C |
| J670 | A | A | C | C |
| J671 | A | A | C | C |
| J672 | A | A | C | C |
| J673 | A | A | C | A |
| J674 | A | A | C | C |
| J675 | A | A | D | A |
| J676 | A | A | C | C |
| J677 | A | A | C | C |
| J678 | A | A | C | C |
| J679 | A | A | C | C |
| J680 | A | A | C | C |
| J681 | A | A | C | C |
| J682 | A | A | C | C |
| J683 | A | A | C | C |
| J684 | A | A | B | C |
| J685 | A | A | C | C |
| J686 | A | A | B | C |
| J687 | A | A | C | C |
| J688 | A | A | C | C |
| J689 | A | A | C | C |
| J690 | A | A | C | C |
| J691 | A | A | C | C |
| J692 | A | A | C | C |
| J693 | A | A | C | C |
| J694 | A | A | C | C |
| J695 | A | A | C | C |
| J696 | A | A | C | C |
| J697 | A | A | C | C |
| J698 | A | A | C | C |
| J699 | A | A | C | C |
| J700 | A | A | C | C |
| J701 | A | A | C | C |
| J702 | A | A | C | C |
| J703 | A | A | C | C |
| J704 | A | A | C | C |
| J705 | A | A | C | C |
| J706 | A | A | C | C |
| J707 | D | D | C | C |
| J708 | A | A | C | A |
| J710 | A | A | C | C |
| J711 | A | A | C | C |
| J712 | A | A | D | A |
| J714 | A | A | D | A |
| J715 | B | D | D | D |
| J719 | A | A | B | A |
| J720 | A | A | D | A |
| J722 | A | A | C | C |
| J723 | A | A | C | C |
| J726 | A | A | C | C |
| J727 | A | A | C | C |
| J728 | A | A | C | C |
| J729 | A | A | C | C |
| J730 | A | A | C | C |
| J732 | A | A | C | C |
| J733 | A | A | C | C |
| J737 | A | A | C | C |
| J738 | A | A | C | C |
| J741 | D | D | D | A |
| J742 | A | A | B | A |
| J746 | A | D | D | A |
| J747 | A | A | B | A |
| J748 | D | D | D | A |
| J749 | C | C | D | C |
| J750 | A | A | C | A |
| J751 | A | A | B | A |
| J752 | A | A | C | C |
| J753 | A | A | C | C |
| J754 | A | A | C | C |
| J755 | A | A | C | C |
| J756 | A | A | C | C |
| J757 | A | A | C | C |
| J758 | A | A | C | C |
| J759 | C | C | C | C |
| J760 | A | B | C | C |
| J761 | A | A | C | C |
| J763 | A | A | C | C |
| J764 | A | A | C | C |
| J765 | A | A | C | C |
| J766 | A | A | C | C |
| J767 | A | A | C | C |
| J768 | A | A | C | C |
| J769 | A | A | C | C |
| J770 | A | A | C | C |
| J771 | A | A | C | C |
| J772 | A | A | C | C |
| J773 | A | A | C | C |
| J774 | C | C | D | D |
| J778 | A | A | C | C |
| J779 | A | A | C | C |
| J780 | A | A | C | C |
| J781 | A | A | C | C |
| J782 | A | A | C | C |
| J783 | A | A | C | C |
| J784 | A | A | C | C |
| J785 | C | C | D | C |
| J786 | A | A | C | C |
| J787 | A | A | C | C |
| J788 | A | A | C | C |
| J789 | A | A | C | C |
| J790 | A | A | C | C |
| J791 | A | A | C | C |
| J792 | A | A | C | C |
| J793 | A | A | C | C |
| J794 | A | A | C | C |
| J795 | A | A | C | C |
| J796 | A | A | C | C |
| J797 | A | A | C | C |
| J798 | A | A | C | C |
| J799 | A | A | C | C |
| J800 | C | C | C | C |
| J801 | A | A | C | C |
| J802 | A | A | C | C |

### Example D: Bioassays on Cabbage Looper (Trichoplusia ni) (“CL”) and Diamondback Moth (Plutella xylostella) (“DBM”)

Certain molecules disclosed in this document were tested against CL and DBM (diamondback moth, Plutella xylostella) using procedures described above for BAW and CL (Example A). The results are reported as in the “CL & DBM Rating Table” at concentrations of 0.0005 and 0.00005 ppm, respectively, and are given Table D.

CL & DBM Rating Table

| % Control (or Mortality) | Rating |
|---|---|
| 51-100 | A |
| 16-50 | B |
| 0-15 | C |

TABLE D
Comparative Biological Results

| Cmpd No | CL | DBM |
|---|---|---|
| C2 | C | C |
| H20 | B | A |
| C3 | C | C |
| H1 | B | B |
| C1 | C | C |
| H2 | A | A |

Compounds of Formula One according to the subject invention are highly efficacious against a variety of pest as can been shown in the comparative data to be superior with previously disclosed compounds. For example, Compound H1 of the subject invention (≥80% control of a range of pests at 0.005 ppm) is superior to Compound N1 (≥80% control of a range of pests at 0.5 ppm) and Compound P49 (and P79) disclosed in U.S. Pat. No. 9,029,560.

| | |
|---|---|
| H1 | 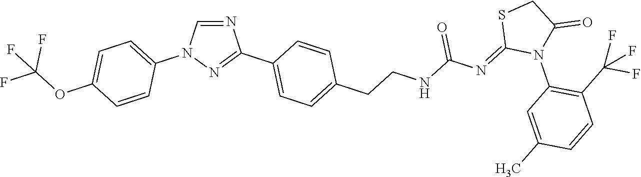 |
| N1 | 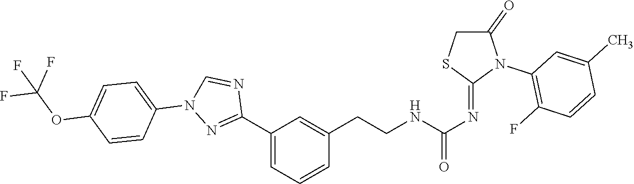 |
| P49 | 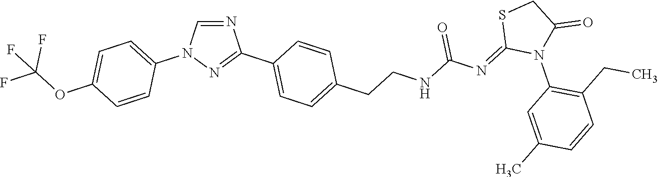 |

In one aspect, provided herein are compound, including for example compounds of Formula One, Formula One-A, and/or Formula Two, wherein R³ is a substituted phenyl having a C₁-C₈ haloalkyl substitution, and said substituted phenyl may have optionally additional one or more substituents independently selected from F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ haloalkyl)O(C₁-C₈ alkyl), (C₁-C₈ haloalkyl)O(C₁-C₈ haloalkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1).

In another aspect, provided herein are compounds, including for example compounds of Formula Three, Formula Four, and/or Formula Five, wherein at least one of R³⁰, R³¹, R³², R³³, and/or R³⁴ must be a C₁-C₈ haloalkyl substitution, and the rest of R³⁰, R³¹, R³², R³³, and/or R³⁴ is independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ haloalkyl)O(C₁-C₈ alkyl), (C₁-C₈ haloalkyl)O(C₁-C₈ haloalkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1).

### Agriculturally Acceptable Acid Addition Salts, Salt Derivatives, Solvates, Ester Derivatives, Polymorphs, Isotopes, and Radionuclides

Molecules of Formula One may be formulated into agriculturally acceptable acid addition salts. By way of a non-limiting example, an amine function can form salts with hydrochloric, hydrobromic, sulfuric, phosphoric, acetic, benzoic, citric, malonic, salicylic, malic, fumaric, oxalic, succinic, tartaric, lactic, gluconic, ascorbic, maleic, aspartic, benzenesulfonic, methanesulfonic, ethanesulfonic, hydroxyl-methanesulfonic, and hydroxyethanesulfonic acids. Additionally, by way of a non-limiting example, an acid function can form salts including those derived from alkali or alkaline earth metals and those derived from ammonia and amines. Examples of preferred cations include sodium, potassium, and magnesium.

Molecules of Formula One may be formulated into salt derivatives. By way of a non-limiting example, a salt derivative can be prepared by contacting a free base with a sufficient amount of the desired acid to produce a salt. A free base may be regenerated by treating the salt with a suitable dilute aqueous base solution such as dilute aqueous sodium hydroxide, potassium carbonate, ammonia, and sodium bicarbonate. As an example, in many cases, a pesticide, such as 2,4-D, is made more water-soluble by converting it to its dimethylamine salt.

Molecules of Formula One may be formulated into stable complexes with a solvent, such that the complex remains intact after the non-complexed solvent is removed. These complexes are often referred to as “solvates.” However, it is particularly desirable to form stable hydrates with water as the solvent.

Molecules of Formula One may be made into ester derivatives. These ester derivatives can then be applied in the same manner as the molecules disclosed in this document is applied.

Molecules of Formula One may be made as various crystal polymorphs. Polymorphism is important in the development of agrochemicals since different crystal polymorphs or structures of the same molecule can have vastly different physical properties and biological performances.

Molecules of Formula One may be made with different isotopes. Of particular importance are molecules having ²H (also known as deuterium) in place of ¹H.

Molecules of Formula One may be made with different radionuclides. Of particular importance are molecules having ¹⁴C.

### Stereoisomers

Molecules of Formula One may exist as one or more stereoisomers. Thus, certain molecules can be produced as racemic mixtures. It will be appreciated by those skilled in the art that one stereoisomer may be more active than the other stereoisomers. Individual stereoisomers may be obtained by known selective synthetic procedures, by conventional synthetic procedures using resolved starting materials, or by conventional resolution procedures. Certain molecules disclosed in this document can exist as two or more isomers. The various isomers include geometric isomers, diastereomers, and enantiomers. Thus, the molecules disclosed in this document include geometric isomers, racemic mixtures, individual stereoisomers, and optically active mixtures. It will be appreciated by those skilled in the art that one isomer may be more active than the others. The structures disclosed in the present disclosure are drawn in only one geometric form for clarity, but are intended to represent all geometric forms of the molecule.

### Combinations

In another embodiment, molecules of Formula One may be used in combination (such as, in a compositional mixture, or a simultaneous or sequential application) with one or more compounds each having a mode of action that is the same as, similar to, or different from, the mode of action (“MoA”) of the molecules of Formula One. Modes of action include, for example the following: Acetylcholinesterase (AChE) inhibitors; GABA-gated chloride channel antagonists; Sodium channel modulators; Nicotinic acetylcholine (nAChR) agonists; Nicotinic acetylcholine receptor (nAChR) allosteric activators; Chloride channel activators; Juvenile hormone mimics; Miscellaneous non-specific (multi-site) inhibitors; Selective homopteran feeding blockers; Mite growth inhibitors; Microbial disruptors of insect midgut membranes; Inhibitors of mitochondrial ATP synthase; Uncouplers of oxidative phosphorylation via disruption of the proton gradient; Nicotinic acetylcholine receptor (nAChR) channel blockers; Inhibitors of chitin biosynthesis, type 0; Inhibitors of chitin biosynthesis, type 1; Moulting disruptor, Dipteran; Ecdysone receptor agonists; Octopamine receptor agonists; Mitochondrial complex III electron transport inhibitors; Mitochondrial complex I electron transport inhibitors; Voltage-dependent sodium channel blockers; Inhibitors of acetyl CoA carboxylase; Mitochondrial complex IV electron transport inhibitors; Mitochondrial complex II electron transport inhibitors; and Ryanodine receptor modulators.

In another embodiment, molecules of Formula One may be used in combination (such as, in a compositional mixture, or a simultaneous or sequential application) with one or more compounds having acaricidal, algicidal, avicidal, bactericidal, fungicidal, herbicidal, insecticidal, molluscicidal, nematicidal, rodenticidal, and/or virucidal properties.

In another embodiment, the molecules of Formula One may be used in combination (such as, in a compositional mixture, or a simultaneous or sequential application) with one or more compounds that are antifeedants, bird repellents, chemosterilants, herbicide safeners, insect attractants, insect repellents, mammal repellents, mating disrupters, plant activators, plant growth regulators, and/or synergists.

In another embodiment, the molecules of Formula One may be used in combination (such as, in a compositional mixture, or a simultaneous or sequential application) with one or more of the following compounds —(3-ethoxypropyl)mercury bromide, 1,2-dichloropropane, 1,3-dichloropropene, 1-methylcyclopropene, 1-naphthol, 2-(octylthio)ethanol, 2,3,5-tri-iodobenzoic acid, 2,3,6-TBA, 2,3,6-TBA-dimethylammonium, 2,3,6-TBA-lithium, 2,3,6-TBA-potassium, 2,3,6-TBA-sodium, 2,4,5-T, 2,4,5-T-2-butoxypropyl, 2,4,5-T-2-ethylhexyl, 2,4,5-T-3-butoxypropyl, 2,4,5-TB, 2,4,5-T-butometyl, 2,4,5-T-butotyl, 2,4,5-T-butyl, 2,4,5-T-isobutyl, 2,4,5-T-isoctyl, 2,4,5-T-isopropyl, 2,4,5-T-methyl, 2,4,5-T-pentyl, 2,4,5-T-sodium, 2,4,5-T-triethylammonium, 2,4,5-T-trolamine, 2,4-D, 2,4-D-2-butoxypropyl, 2,4-D-2-ethylhexyl, 2,4-D-3-butoxypropyl, 2,4-D-ammonium, 2,4-DB, 2,4-DB-butyl, 2,4-DB-dimethylammonium, 2,4-DB-isoctyl, 2,4-DB-potassium, 2,4-DB-sodium, 2,4-D-butotyl, 2,4-D-butyl, 2,4-D-diethylammonium, 2,4-D-dimethylammonium, 2,4-D-diolamine, 2,4-D-dodecylammonium, 2,4-DEB, 2,4-DEP, 2,4-D-ethyl, 2,4-D-heptylammonium, 2,4-D-isobutyl, 2,4-D-isoctyl, 2,4-D-isopropyl, 2,4-D-isopropylammonium, 2,4-D-lithium, 2,4-D-meptyl, 2,4-D-methyl, 2,4-D-octyl, 2,4-D-pentyl, 2,4-D-potassium, 2,4-D-propyl, 2,4-D-sodium, 2,4-D-tefuryl, 2,4-D-tetradecylammonium, 2,4-D-triethylammonium, 2,4-D-tris(2-hydroxypropyl)ammonium, 2,4-D-trolamine, 2iP, 2-methoxyethylmercury chloride, 2-phenylphenol, 3,4-DA, 3,4-DB, 3,4-DP, 4-aminopyridine, 4-CPA, 4-CPA-diolamine, 4-CPA-potassium, 4-CPA-sodium, 4-CPB, 4-CPP, 4-hydroxyphenethyl alcohol, 8-hydroxyquinoline sulfate, 8-phenylmercurioxyquinoline, abamectin, abscisic acid, ACC, acephate, acequinocyl, acetamiprid, acethion, acetochlor, acetophos, acetoprole, acibenzolar, acibenzolar-S-methyl, acifluorfen, acifluorfen-methyl, acifluorfen-sodium, aclonifen, acrep, acrinathrin, acrolein, acrylonitrile, acynonapyr, acypetacs, acypetacs-copper, acypetacs-zinc, afidopyropen, afoxolaner, alachlor, alanycarb, albendazole, aldicarb, aldimorph, aldoxycarb, aldrin, allethrin, allicin, allidochlor, allosamidin, alloxydim, alloxydim-sodium, allyl alcohol, allyxycarb, alorac, alpha-cypermethrin, alpha-endosulfan, ametoctradin, ametridione, ametryn, amibuzin, amicarbazone, amicarthiazol, amidithion, amidoflumet, amidosulfuron, aminocarb, aminocyclopyrachlor, aminocyclopyrachlor-methyl, aminocyclopyrachlor-potassium, aminopyralid, aminopyralid-potassium, aminopyralid-tris(2-hydroxypropyl)ammonium, aminopyrifen, amiprofos-methyl, amiprophos, amisulbrom, amiton, amiton oxalate, amitraz, amitrole, ammonium sulfamate, ammonium α-naphthaleneacetate, amobam, ampropylfos, anabasine, anabasine sulfate, ancymidol, anilazine, anilofos, anisuron, anthraquinone, antu, apholate, aramite, arsenous oxide, asomate, aspirin, asulam, asulam-potassium, asulam-sodium, athidathion, atraton, atrazine, aureofungin, aviglycine, aviglycine hydrochloride, azaconazole, azadirachtin, azafenidin, azamethiphos, azimsulfuron, azinphos-ethyl, azinphos-methyl, aziprotryne, azithiram, azobenzene, azocyclotin, azothoate, azoxystrobin, bachmedesh, barban, barium hexafluorosilicate, barium polysulfide, barthrin, BCPC, beflubutamid, beflubutamid-M, benalaxyl, benalaxyl-M, benazolin, benazolin-dimethylammonium, benazolin-ethyl, benazolin-potassium, bencarbazone, benclothiaz, bendiocarb, benfluralin, benfuracarb, benfuresate, benodanil, benomyl, benoxacor, benoxafos, benquinox, bensulfuron, bensulfuron-methyl, bensulide, bensultap, bentaluron, bentazone, bentazone-sodium, benthiavalicarb, benthiavalicarb-isopropyl, benthiazole, bentranil, benzadox, benzadox-ammonium, benzalkonium chloride, benzamacril, benzamacril-isobutyl, benzamorf, benzfendizone, benzipram, benzobicyclon, benzofenap, benzofluor, benzohydroxamic acid, benzovindiflupyr, benzoximate, benzoylprop, benzoylprop-ethyl, benzpyrimoxan, benzthiazuron, benzyl benzoate, benzyladenine, berberine, berberine chloride, beta-cyfluthrin, beta-cypermethrin, bethoxazin, bicyclopyrone, bifenazate, bifenox, bifenthrin, bifujunzhi, bilanafos, bilanafos-sodium, binapacryl, bingqingxiao, bioallethrin, bioethanomethrin, biopermethrin, bioresmethrin, biphenyl, bisazir, bismerthiazol, bispyribac, bispyribac-sodium, bistrifluron, bitertanol, bithionol, bixafen, bixlozone, blasticidin-S, borax, Bordeaux mixture, boric acid, boscalid, brassinolide, brassinolide-ethyl, brevicomin, brodifacoum, brofenvalerate, broflanilide, brofluthrinate, bromacil, bromacil-lithium, bromacil-sodium, alpha-bromadiolone, bromadiolone, bromethalin, bromethrin, bromfenvinfos, bromoacetamide, bromobonil, bromobutide, bromocyclen, bromo-DDT, bromofenoxim, bromophos, bromophos-ethyl, bromopropylate, bromothalonil, bromoxynil, bromoxynil butyrate, bromoxynil heptanoate, bromoxynil octanoate, bromoxynil-potassium, brompyrazon, bromuconazole, bronopol, bucarpolate, bufencarb, buminafos, bupirimate, buprofezin, Burgundy mixture, busulfan, butacarb, butachlor, butafenacil, butamifos, butathiofos, butenachlor, butethrin, buthidazole, buthiobate, buthiuron, butocarboxim, butonate, butopyronoxyl, butoxycarboxim, butralin, butroxydim, buturon, butylamine, butylate, cacodylic acid, cadusafos, cafenstrole, calcium arsenate, calcium chlorate, calcium cyanamide, calcium polysulfide, calvinphos, cambendichlor, camphechlor, camphor, captafol, captan, carbamorph, carbanolate, carbaryl, carbasulam, carbendazim, carbendazim benzenesulfonate, carbendazim sulfite, carbetamide, carbofuran, carbon disulfide, carbon tetrachloride, carbophenothion, carbosulfan, carboxazole, carboxide, carboxin, carfentrazone, carfentrazone-ethyl, carpropamid, cartap, cartap hydrochloride, carvacrol, carvone, CDEA, cellocidin, CEPC, ceralure, Cheshunt mixture, chinomethionat, chitosan, chlobenthiazone, chlomethoxyfen, chloralose, chloramben, chloramben-ammonium, chloramben-diolamine, chloramben-methyl, chloramben-methylammonium, chloramben-sodium, chloramine phosphorus, chloramphenicol, chloraniformethan, chloranil, chloranocryl, chlorantraniliprole, chlorazifop, chlorazifop-propargyl, chlorazine, chlorbenside, chlorbenzuron, chlorbicyclen, chlorbromuron, chlorbufam, chlordane, chlordecone, chlordimeform, chlordimeform hydrochloride, chlorempenthrin, chlorethoxyfos, chloreturon, chlorfenac, chlorfenac-ammonium, chlorfenac-sodium, chlorfenapyr, chlorfenazole, chlorfenethol, chlorfenprop, chlorfenson, chlorfensulphide, chlorfenvinphos, chlorfluazuron, chlorflurazole, chlorfluren, chlorfluren-methyl, chlorflurenol, chlorflurenol-methyl, chloridazon, chlorimuron, chlorimuron-ethyl, chlormephos, chlormequat, chlormequat chloride, chlornidine, chlornitrofen, chlorobenzilate, chlorodinitronaphthalenes, chloroform, chloromebuform, chloromethiuron, chloroneb, chlorophacinone, chlorophacinone-sodium, chloropicrin, chloropon, chloroprallethrin, chloropropylate, chlorothalonil, chlorotoluron, chloroxuron, chloroxynil, chlorphonium, chlorphonium chloride, chlorphoxim, chlorprazophos, chlorprocarb, chlorpropham, chlorpyrifos, chlorpyrifos-methyl, chlorquinox, chlorsulfuron, chlorthal, chlorthal-dimethyl, chlorthal-monomethyl, chlorthiamid, chlorthiophos, chlozolinate, choline chloride, chromafenozide, cinerin I, cinerin II, cinerins, cinidon-ethyl, cinmethylin, cinosulfuron, ciobutide, cisanilide, cismethrin, clacyfos, clethodim, climbazole, cliodinate, clodinafop, clodinafop-propargyl, cloethocarb, clofencet, clofencet-potassium, clofentezine, clofibric acid, clofop, clofop-isobutyl, clomazone, clomeprop, cloprop, cloproxydim, clopyralid, clopyralid-methyl, clopyralid-olamine, clopyralid-potassium, clopyralid-tris(2-hydroxypropyl)ammonium, cloquintocet, cloquintocet-mexyl, cloransulam, cloransulam-methyl, closantel, clothianidin, clotrimazole, cloxyfonac, cloxyfonac-sodium, CMA, codlelure, colophonate, copper acetate, copper acetoarsenite, copper arsenate, copper carbonate, basic, copper hydroxide, copper naphthenate, copper oleate, copper oxychloride, copper silicate, copper sulfate, copper zinc chromate, coumachlor, coumafuryl, coumaphos, coumatetralyl, coumithoate, coumoxystrobin, coumoxystrobin, CPMC, CPMF, CPPC, credazine, cresol, crimidine, crotamiton, crotoxyphos, crufomate, cryolite, cue-lure, cufraneb, cumyluron, cuprobam, cuprous oxide, curcumenol, cyanamide, cyanatryn, cyanazine, cyanofenphos, cyanophos, cyanthoate, cyantraniliprole, cyazofamid, cybutryne, cyclafuramid, cyclanilide, cyclaniliprole, cyclethrin, cycloate, cyclobutrifluram, cycloheximide, cycloprate, cycloprothrin, cyclopyranil, cyclopyrimorate, cyclosulfamuron, cycloxaprid, cycloxydim, cycluron, cyenopyrafen, cyetpyrafen, cyflufenamid, cyflumetofen, cyfluthrin, cyhalodiamide, cyhalofop, cyhalofop-butyl, cyhalothrin, cyhexatin, cymiazole, cymiazole hydrochloride, cymoxanil, cyometrinil, cypendazole, cypermethrin, cyperquat, cyperquat chloride, cyphenothrin, cyprazine, cyprazole, cyproconazole, cyprodinil, cyproflanilide, cyprofuram, cypromid, cyprosulfamide, cyromazine, cythioate, daimuron, dalapon, dalapon-calcium, dalapon-magnesium, dalapon-sodium, daminozide, dayoutong, dazomet, dazomet-sodium, DBCP, d-camphor, DCIP, DCPTA, DDT, debacarb, decafentin, decarbofuran, dehydroacetic acid, delachlor, deltamethrin, demephion, demephion-O, demephion-S, demeton, demeton-methyl, demeton-O, demeton-O-methyl, demeton-S, demeton-S-methyl, demeton-S-methylsulphon, desmedipham, desmetryn, d-fanshiluquebingjuzhi, diafenthiuron, dialifos, di-allate, diamidafos, diatomaceous earth, diazinon, dibutyl phthalate, dibutyl succinate, dicamba, dicamba-diglycolamine, dicamba-dimethylammonium, dicamba-diolamine, dicamba-isopropylammonium, dicamba-methyl, dicamba-olamine, dicamba-potassium, dicamba-sodium, dicamba-trolamine, dicapthon, dichlobenil, dichlobentiazox, dichlofenthion, dichlofluanid, dichlone, dichloralurea, dichlorbenzuron, dichlorflurenol, dichlorflurenol-methyl, dichlormate, dichlormid, dichlorophen, dichlorprop, dichlorprop-2-ethylhexyl, dichlorprop-butotyl, dichlorprop-dimethylammonium, dichlorprop-ethylammonium, dichlorprop-isoctyl, dichlorprop-methyl, dichlorprop-P, dichlorprop-P-2-ethylhexyl, dichlorprop-P-dimethylammonium, dichlorprop-potassium, dichlorprop-P-potassium, dichlorprop-P-sodium, dichlorprop-sodium, dichlorvos, dichlozoline, diclobutrazol, diclocymet, diclofop, diclofop-methyl, diclomezine, diclomezine-sodium, dicloran, dicloromezotiaz, diclosulam, dicofol, dicoumarol, dicresyl, dicrotophos, dicyclanil, dicyclonon, dieldrin, dienochlor, diethamquat, diethamquat dichloride, diethatyl, diethatyl-ethyl, diethofencarb, dietholate, diethyl pyrocarbonate, diethyltoluamide, difenacoum, difenoconazole, difenopenten, difenopenten-ethyl, difenoxuron, difenzoquat, difenzoquat metilsulfate, difethialone, diflovidazin, diflubenzuron, diflufenican, diflufenzopyr, diflufenzopyr-sodium, diflumetorim, dikegulac, dikegulac-sodium, dilor, dimatif, dimefluthrin, dimefox, dimefuron, dimepiperate, dimetachlone, dimetan, dimethacarb, dimethachlor, dimethametryn, dimethenamid, dimethenamid-P, dimethipin, dimethirimol, dimethoate, dimethomorph, dimethrin, dimethyl carbate, dimethyl phthalate, dimethylvinphos, dimetilan, dimexano, dimidazon, dimoxystrobin, dimpropyridaz, dinex, dinex-diclexine, dingjunezuo, diniconazole, diniconazole-M, dinitramine, dinobuton, dinocap, dinocap-4, dinocap-6, dinocton, dinofenate, dinopenton, dinoprop, dinosam, dinoseb, dinoseb acetate, dinoseb-ammonium, dinoseb-diolamine, dinoseb-sodium, dinoseb-trolamine, dinosulfon, dinotefuran, dinoterb, dinoterb acetate, dinoterbon, diofenolan, dioxabenzofos, dioxacarb, dioxathion, diphacinone, diphacinone-sodium, diphenamid, diphenyl sulfone, diphenylamine, dipropalin, dipropetryn, dipymetitrone, dipyrithione, diquat, diquat dibromide, disparlure, disul, disulfiram, disulfoton, disul-sodium, ditalimfos, dithianon, dithicrofos, dithioether, dithiopyr, diuron, d-limonene, DMPA, DNOC, DNOC-ammonium, DNOC-potassium, DNOC-sodium, dodemorph, dodemorph acetate, dodemorph benzoate, dodicin, dodicin hydrochloride, dodicin-sodium, dodine, dofenapyn, dominicalure, doramectin, drazoxolon, DSMA, dufulin, EBEP, EBP, ecdysterone, edifenphos, eglinazine, eglinazine-ethyl, emamectin, emamectin benzoate, EMPC, empenthrin, endosulfan, endothal, endothal-diammonium, endothal-dipotassium, endothal-disodium, endothion, endrin, enestroburin, enoxastrobin, EPN, epocholeone, epofenonane, epoxiconazole, eprinomectin, epronaz, epsilon-metofluthrin, epsilon-momfluorothrin, EPTC, erbon, ergocalciferol, erlujixiancaoan, esdepallethrine, esfenvalerate, esprocarb, etacelasil, etaconazole, etaphos, etem, ethaboxam, ethachlor, ethalfluralin, ethametsulfuron, ethametsulfuron-methyl, ethaprochlor, ethephon, ethidimuron, ethiofencarb, ethiolate, ethion, ethiozin, ethiprole, ethirimol, ethoate-methyl, ethofumesate, ethohexadiol, ethoprophos, ethoxyfen, ethoxyfen-ethyl, ethoxyquin, ethoxysulfuron, ethychlozate, ethyl formate, ethyl α-naphthaleneacetate, ethyl-DDD, ethylene, ethylene dibromide, ethylene dichloride, ethylene oxide, ethylicin, ethylmercury 2,3-dihydroxypropyl mercaptide, dimesulfazet, epyrifenacil, ethylmercury acetate, ethylmercury bromide, ethylmercury chloride, ethylmercury phosphate, etinofen, etnipromid, etobenzanid, etofenprox, etoxazole, etridiazole, etrimfos, eugenol, EXD, famoxadone, famphur, fenamidone, fenaminosulf, fenaminstrobin, fenamiphos, fenapanil, fenarimol, fenasulam, fenazaflor, fenazaquin, fenbuconazole, fenbutatin oxide, fenchlorazole, fenchlorazole-ethyl, fenchlorphos, fenclorim, fenethacarb, fenfluthrin, fenfuram, fenhexamid, fenitropan, fenitrothion, fenjuntong, fenobucarb, fenoprop, fenoprop-3-butoxypropyl, fenoprop-butometyl, fenoprop-butotyl, fenoprop-butyl, fenoprop-isoctyl, fenoprop-methyl, fenoprop-potassium, fenothiocarb, fenoxacrim, fenoxanil, fenoxaprop, fenoxaprop-ethyl, fenoxaprop-P, fenoxaprop-P-ethyl, fenoxasulfone, fenoxycarb, fenpiclonil, fenpicoxamid, fenpirithrin, fenpropathrin, fenpropidin, fenpropimorph, fenpyrazamine, fenpyroximate, fenquinotrione, fenridazon, fenridazon-potassium, fenridazon-propyl, fenson, fensulfothion, fenteracol, fenthiaprop, fenthiaprop-ethyl, fenthion, fenthion-ethyl, fentin, fentin acetate, fentin chloride, fentin hydroxide, fentrazamide, fentrifanil, fenuron, fenuron TCA, fenvalerate, ferbam, ferimzone, ferrous sulfate, fipronil, flamprop, flamprop-isopropyl, flamprop-M, flamprop-methyl, flamprop-M-isopropyl, flamprop-M-methyl, flazasulfuron, flocoumafen, flometoquin, flonicamid, florasulam, florpyrauxifen, florylpicoxamid, fluacrypyrim, fluazaindolizine, fluazifop, fluazifop-butyl, fluazifop-methyl, fluazifop-P, fluazifop-P-butyl, fluazinam, fluazolate, fluazuron, flubendiamide, flubeneteram, flubenzimine, flucarbazone, flucarbazone-sodium, flucetosulfuron, fluchloralin, flucofuron, flucycloxuron, flucythrinate, fludioxonil, fluenetil, fluensulfone, flufenacet, flufenerim, flufenican, flufenoxuron, flufenoxystrobin, flufenprox, flufenpyr, flufenpyr-ethyl, flufiprole, fluhexafon, fluindapyr, flumethrin, flumetover, flumetralin, flumetsulam, flumezin, flumiclorac, flumiclorac-pentyl, flumioxazin, flumipropyn, flumorph, fluometuron, fluopicolide, fluopimomide, fluopyram, fluorbenside, fluoridamid, fluoroacetamide, fluorodifen, fluoroglycofen, fluoroglycofen-ethyl, fluoroimide, fluoromidine, fluoronitrofen, fluothiuron, fluotrimazole, fluoxapiprolin, fluoxastrobin, flupentiofenox, flupoxam, flupropacil, flupropadine, flupropanate, flupropanate-sodium, flupyradifurone, flupyrimin, flupyrsulfuron, flupyrsulfuron-methyl, flupyrsulfuron-methyl-sodium, fluquinconazole, fluralaner, flurazole, flurenol, flurenol-butyl, flurenol-methyl, fluridone, flurochloridone, fluroxypyr, fluroxypyr-butometyl, fluroxypyr-meptyl, flurprimidol, flursulamid, flurtamone, flusilazole, flusulfamide, fluthiacet, fluthiacet-methyl, flutianil, flutolanil, flutriafol, fluvalinate, fluxametamide, fluxapyroxad, fluxofenim, folpet, fomesafen, fomesafen-sodium, fonofos, foramsulfuron, forchlorfenuron, formaldehyde, formetanate, formetanate hydrochloride, formothion, formparanate, formparanate hydrochloride, fosamine, fosamine-ammonium, fosetyl, fosetyl-aluminium, fosmethilan, fospirate, fosthiazate, fosthietan, frontalin, fuberidazole, fucaojing, fucaomi, funaihecaoling, fuphenthiourea, furalane, furalaxyl, furamethrin, furametpyr, furathiocarb, furcarbanil, furconazole, furconazole-cis, furethrin, furfural, furilazole, furmecyclox, furophanate, furyloxyfen, gamma-cyhalothrin, gamma-HCH, genit, gibberellic acid, gibberellins, gliftor, glufosinate, glufosinate-ammonium, glufosinate-P, glufosinate-P-ammonium, glufosinate-P-sodium, glyodin, glyoxime, glyphosate, glyphosate-diammonium, glyphosate-dimethylammonium, glyphosate-isopropylammonium, glyphosate-monoammonium, glyphosate-potassium, glyphosate-sesquisodium, glyphosate-trimesium, glyphosine, gossyplure, grandlure, griseofulvin, guazatine, guazatine acetates, halacrinate, halauxifen, halauxifen-methyl, halfenprox, halofenozide, halosafen, halosulfuron, halosulfuron-methyl, haloxydine, haloxyfop, haloxyfop-etotyl, haloxyfop-methyl, haloxyfop-P, haloxyfop-P-etotyl, haloxyfop-P-methyl, haloxyfop-sodium, HCH, hemel, hempa, HEOD, heptachlor, heptafluthrin, heptenophos, heptopargil, herbimycin, heterophos, hexachloroacetone, hexachlorobenzene, hexachlorobutadiene, hexachlorophene, hexaconazole, hexaflumuron, hexaflurate, hexalure, hexamide, hexazinone, hexylthiofos, hexythiazox, HHDN, holosulf, huancaiwo, huangcaoling, huanjunzuo, hydramethylnon, hydrargaphen, hydrated lime, hydrogen cyanide, hydroprene, hymexazol, hyquincarb, IAA, IBA, icaridin, imazalil, imazalil nitrate, imazalil sulfate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazaquin-methyl, imazaquin-sodium, imazethapyr, imazethapyr-ammonium, imazosulfuron, imibenconazole, imicyafos, imidacloprid, imidaclothiz, iminoctadine, iminoctadine triacetate, iminoctadine trialbesilate, imiprothrin, inabenfide, indanofan, indaziflam, indoxacarb, inezin, inpyrfluxam, iodobonil, iodocarb, iodomethane, iodosulfuron, iodosulfuron-methyl, iodosulfuron-methyl-sodium, iofensulfuron, iofensulfuron-sodium, ioxynil, ioxynil octanoate, ioxynil-lithium, ioxynil-sodium, ipazine, ipconazole, ipfencarbazone, ipfentrifluconazole, iprobenfos, iprodione, iprovalicarb, iprymidam, ipsdienol, ipsenol, IPSP, isamidofos, isazofos, isobenzan, isocarbamid, isocarbophos, isocil, isocycloseram, isodrin, isofenphos, isofenphos-methyl, isofetamid, isoflucypram, ipflufenoquin, isolan, isomethiozin, isonoruron, isopolinate, isoprocarb, isopropalin, isoprothiolane, isoproturon, isopyrazam, isopyrimol, isothioate, isotianil, isouron, isovaledione, isoxaben, isoxachlortole, isoxadifen, isoxadifen-ethyl, isoxaflutole, isoxapyrifop, isoxathion, ivermectin, izopamfos, japonilure, japothrins, jasmolin I, jasmolin II, jasmonic acid, jiahuangchongzong, jiajizengxiaolin, jiaxiangjunzhi, jiecaowan, jiecaoxi, jodfenphos, juvenile hormone I, juvenile hormone II, juvenile hormone III, kadethrin, kappa-bifenthrin, kappa-tefluthrin, karbutilate, karetazan, karetazan-potassium, kasugamycin, kasugamycin hydrochloride, kejunlin, kelevan, ketospiradox, ketospiradox-potassium, kinetin, kinoprene, kresoxim-methyl, kuicaoxi, lactofen, lambda-cyhalothrin, lancotrione, latilure, lead arsenate, lenacil, lepimectin, leptophos, lindane, lineatin, linuron, lirimfos, litlure, looplure, lotilaner, lufenuron, Ivdingjunzhi, Ivxiancaolin, lythidathion, MAA, malathion, maleic hydrazide, malonoben, maltodextrin, MAMA, mancopper, mancozeb, mandestrobin, mandipropamid, maneb, matrine, mazidox, MCPA, MCPA-2-ethylhexyl, MCPA-butotyl, MCPA-butyl, MCPA-dimethylammonium, MCPA-diolamine, MCPA-ethyl, MCPA-isobutyl, MCPA-isoctyl, MCPA-isopropyl, MCPA-methyl, MCPA-olamine, MCPA-potassium, MCPA-sodium, MCPA-thioethyl, MCPA-trolamine, MCPB, MCPB-ethyl, MCPB-methyl, MCPB-sodium, mebenil, mecarbam, mecarbinzid, mecarphon, mecoprop, mecoprop-2-ethylhexyl, mecoprop-dimethylammonium, mecoprop-diolamine, mecoprop-ethadyl, mecoprop-isoctyl, mecoprop-methyl, mecoprop-P, mecoprop-P-2-ethylhexyl, mecoprop-P-dimethylammonium, mecoprop-P-isobutyl, mecoprop-potassium, mecoprop-P-potassium, mecoprop-sodium, mecoprop-trolamine, medimeform, medinoterb, medinoterb acetate, medlure, mefenacet, mefenpyr, mefenpyr-diethyl, mefentrifluconazole, mefluidide, mefluidide-diolamine, mefluidide-potassium, megatomoic acid, menazon, mepanipyrim, meperfluthrin, mephenate, mephosfolan, mepiquat, mepiquat chloride, mepiquat pentaborate, mepronil, meptyldinocap, mercuric chloride, mercuric oxide, mercurous chloride, merphos, mesoprazine, mesosulfuron, mesosulfuron-methyl, mesotrione, mesulfen, mesulfenfos, metcamifen, metaflumizone, metalaxyl, metalaxyl-M, metaldehyde, metam, metam-ammonium, metamifop, metamitron, metam-potassium, metam-sodium, metazachlor, metazosulfuron, metazoxolon, metconazole, metepa, metflurazon, methabenzthiazuron, methacrifos, methalpropalin, methamidophos, methasulfocarb, methazole, methfuroxam, methidathion, methiobencarb, methiocarb, methiopyrisulfuron, methiotepa, methiozolin, methiuron, methocrotophos, methometon, methomyl, methoprene, methoprotryne, methoquin-butyl, methothrin, methoxychlor, methoxyfenozide, methoxyphenone, methyl apholate, methyl bromide, methyl eugenol, methyl iodide, methyl isothiocyanate, methylacetophos, methylchloroform, methyldymron, methylene chloride, methylmercury benzoate, methylmercury dicyandiamide, methylmercury pentachlorophenoxide, methylneodecanamide, metiram, metobenzuron, metobromuron, metofluthrin, metolachlor, metolcarb, metominostrobin, metosulam, metoxadiazone, metoxuron, metrafenone, metribuzin, metsulfovax, metsulfuron, metsulfuron-methyl, metyltetrapole, mevinphos, mexacarbate, mieshuan, milbemectin, milbemycin oxime, milneb, mipafox, mirex, MNAF, moguchun, molinate, molosultap, momfluorothrin, monalide, monisouron, monochloroacetic acid, monocrotophos, monolinuron, monosulfuron, monosulfuron-ester, monuron, monuron TCA, morfamquat, morfamquat dichloride, moroxydine, moroxydine hydrochloride, morphothion, morzid, moxidectin, MSMA, muscalure, myclobutanil, myclozolin, N-(ethylmercury)-p-toluenesulphonanilide, nabam, naftalofos, naled, naphthalene, naphthaleneacetamide, naphthalic anhydride, naphthoxyacetic acids, naproanilide, napropamide, napropamide-M, naptalam, naptalam-sodium, natamycin, neburon, niclosamide, niclosamide-olamine, nicofluprole, nicosulfuron, nicotine, nifluridide, nipyraclofen, nitenpyram, nithiazine, nitralin, nitrapyrin, nitrilacarb, nitrofen, nitrofluorfen, nitrostyrene, nitrothal-isopropyl, norbormide, norflurazon, nornicotine, noruron, novaluron, noviflumuron, nuarimol, OCH, octachlorodipropyl ether, octhilinone, ofurace, omethoate, orbencarb, orfralure, ortho-dichlorobenzene, orthosulfamuron, oryctalure, orysastrobin, oryzalin, osthol, ostramone, oxabetrinil, oxadiargyl, oxadiazon, oxadixyl, oxamate, oxamyl, oxapyrazon, oxapyrazon-dimolamine, oxapyrazon-sodium, oxasulfuron, oxathiapiprolin, oxaziclomefone, oxazosulfyl, oxine-copper, oxolinic acid, oxpoconazole, oxpoconazole fumarate, oxycarboxin, oxydemeton-methyl, oxydeprofos, oxydisulfoton, oxyfluorfen, oxymatrine, oxytetracycline, oxytetracycline hydrochloride, paclobutrazol, paichongding, para-dichlorobenzene, parafluron, paraquat, paraquat dichloride, paraquat dimetilsulfate, parathion, parathion-methyl, parinol, pebulate, pefurazoate, pelargonic acid, penconazole, pencycuron, pendimethalin, penflufen, penfluron, penoxsulam, pentachlorophenol, pentachlorophenyl laurate, pentanochlor, penthiopyrad, pentmethrin, pentoxazone, perfluidone, permethrin, pethoxamid, phenamacril, phenazine oxide, phenisopham, phenkapton, phenmedipham, phenmedipham-ethyl, phenobenzuron, phenothrin, phenproxide, phenthoate, phenylmercuriurea, phenylmercury acetate, phenylmercury chloride, phenylmercury derivative of pyrocatechol, phenylmercury nitrate, phenylmercury salicylate, phorate, phosacetim, phosalone, phosdiphen, phosfolan, phosfolan-methyl, phosglycin, phosmet, phosnichlor, phosphamidon, phosphine, phosphocarb, phosphorus, phostin, phoxim, phoxim-methyl, phthalide, picarbutrazox, picloram, picloram-2-ethylhexyl, picloram-isoctyl, picloram-methyl, picloram-olamine, picloram-potassium, picloram-triethylammonium, picloram-tris(2-hydroxypropyl)ammonium, picolinafen, picoxystrobin, pindone, pindone-sodium, pinoxaden, piperalin, piperonyl butoxide, piperonyl cyclonene, piperophos, piproctanyl, piproctanyl bromide, piprotal, pirimetaphos, pirimicarb, pirimioxyphos, pirimiphos-ethyl, pirimiphos-methyl, plifenate, polycarbamate, polyoxins, polyoxorim, polyoxorim-zinc, polythialan, potassium arsenite, potassium azide, potassium cyanate, potassium gibberellate, potassium naphthenate, potassium polysulfide, potassium thiocyanate, potassium α-naphthaleneacetate, pp′-DDT, prallethrin, precocene I, precocene II, precocene III, pretilachlor, primidophos, primisulfuron, primisulfuron-methyl, probenazole, prochloraz, prochloraz-manganese, proclonol, procyazine, procymidone, prodiamine, profenofos, profluazol, profluralin, profluthrin, profoxydim, proglinazine, proglinazine-ethyl, prohexadione, prohexadione-calcium, prohydrojasmon, promacyl, promecarb, prometon, prometryn, promurit, pronitridine, propachlor, propamidine, propamidine dihydrochloride, propamocarb, propamocarb hydrochloride, propanil, propaphos, propaquizafop, propargite, proparthrin, propazine, propetamphos, propham, propiconazole, propineb, propisochlor, propoxur, propoxycarbazone, propoxycarbazone-sodium, propyl isome, propyrisulfuron, propyzamide, proquinazid, prosuler, prosulfalin, prosulfocarb, prosulfuron, prothidathion, prothiocarb, prothiocarb hydrochloride, prothioconazole, prothiofos, prothoate, protrifenbute, proxan, proxan-sodium, prynachlor, pydanon, pydiflumetofen, pyflubumide, pymetrozine, pyracarbolid, pyraclofos, pyraclonil, pyraclostrobin, pyraflufen, pyraflufen-ethyl, pyrafluprole, pyramat, pyrametostrobin, pyraoxystrobin, pyrapropoyne, pyrasulfotole, pyraziflumid, pyrazolynate, pyrazophos, pyrazosulfuron, pyrazosulfuron-ethyl, pyrazothion, pyrazoxyfen, pyresmethrin, pyrethrin I, pyrethrin II, pyrethrins, pyribambenz-isopropyl, pyribambenz-propyl, pyribencarb, pyribenzoxim, pyributicarb, pyriclor, pyridaben, pyridachlometyl, pyridafol, pyridalyl, pyridaphenthion, pyridate, pyridinitril, pyrifenox, pyrifluquinazon, pyriftalid, pyrimethanil, pyrimidifen, pyriminobac, pyriminobac-methyl, pyriminostrobin, pyrimisulfan, pyrimitate, pyrinuron, pyriofenone, pyriprole, pyripropanol, pyriproxyfen, pyrisoxazole, pyrithiobac, pyrithiobac-sodium, pyrolan, pyroquilon, pyroxasulfone, pyroxsulam, pyroxychlor, pyroxyfur, quassia, quinacetol, quinacetol sulfate, quinalphos, quinalphos-methyl, quinazamid, quinclorac, quinconazole, quinmerac, quinoclamine, quinofumelin, quinonamid, quinothion, quinoxyfen, quintiofos, quintozene, quizalofop, quizalofop-ethyl, quizalofop-P, quizalofop-P-ethyl, quizalofop-P-tefuryl, quwenzhi, quyingding, rabenzazole, rafoxanide, rebemide, rescalure, resmethrin, rhodethanil, rhodojaponin-III, ribavirin, rimsulfuron, rotenone, ryania, saflufenacil, saijunmao, saisentong, salicylanilide, sanguinarine, santonin, sarolaner, schradan, scilliroside, sebuthylazine, secbumeton, sedaxane, selamectin, semiamitraz, semiamitraz chloride, sesamex, sesamolin, sethoxydim, shuangjiaancaolin, siduron, siglure, silafluofen, silatrane, silica gel, silthiofam, simazine, simeconazole, simeton, simetryn, sintofen, SMA, S-metolachlor, sodium arsenite, sodium azide, sodium chlorate, sodium fluoride, sodium fluoroacetate, sodium hexafluorosilicate, sodium naphthenate, sodium orthophenylphenoxide, sodium pentachlorophenoxide, sodium polysulfide, sodium thiocyanate, sodium α-naphthaleneacetate, sophamide, spinetoram, spinosad, spirodiclofen, spiromesifen, spiropidion, spirotetramat, spiroxamine, streptomycin, streptomycin sesquisulfate, strychnine, sulcatol, sulcofuron, sulcofuron-sodium, sulcotrione, sulfallate, sulfentrazone, sulfiram, sulfluramid, sulfometuron, sulfometuron-methyl, sulfosulfuron, sulfotep, sulfoxaflor, sulfoxide, sulfoxime, sulfur, sulfuric acid, sulfuryl fluoride, sulglycapin, sulprofos, sultropen, swep, tau-fluvalinate, tavron, tazimcarb, TCA, TCA-ammonium, TCA-calcium, TCA-ethadyl, TCA-magnesium, TCA-sodium, TDE, tebuconazole, tebufenozide, tebufenpyrad, tebufloquin, tebupirimfos, tebutam, tebuthiuron, tecloftalam, tecnazene, tecoram, teflubenzuron, tefluthrin, tefuryltrione, tembotrione, temephos, tepa, TEPP, tepraloxydim, terallethrin, terbacil, terbucarb, terbuchlor, terbufos, terbumeton, terbuthylazine, terbutryn, tetcyclacis, tetflupyrolimet, tetrachlorantraniliprole, tetrachloroethane, tetrachlorvinphos, tetraconazole, tetradifon, tetrafluron, tetramethrin, tetramethylfluthrin, tetramine, tetranactin, tetraniliprole, tetrasul, thallium sulfate, thenylchlor, theta-cypermethrin, thiabendazole, thiacloprid, thiadifluor, thiamethoxam, thiapronil, thiazafluron, thiazopyr, thicrofos, thicyofen, thidiazimin, thidiazuron, thiencarbazone, thiencarbazone-methyl, thifensulfuron, thifensulfuron-methyl, thifluzamide, thiobencarb, thiocarboxime, thiochlorfenphim, thiocyclam, thiocyclam hydrochloride, thiocyclam oxalate, thiodiazole-copper, thiodicarb, thiofanox, thiofluoximate, thiohempa, thiomersal, thiometon, thionazin, thiophanate, thiophanate-methyl, thioquinox, thiosemicarbazide, thiosultap, thiosultap-diammonium, thiosultap-disodium, thiosultap-monosodium, thiotepa, thiram, thuringiensin, tiadinil, tiafenacil, tiaojiean, tiocarbazil, tioclorim, tioxazafen, tioxymid, tirpate, tolclofos-methyl, tolfenpyrad, tolprocarb, tolpyralate, tolylfluanid, tolylmercury acetate, topramezone, tralkoxydim, tralocythrin, tralomethrin, tralopyril, transfluthrin, transpermethrin, tretamine, triacontanol, triadimefon, triadimenol, triafamone, tri-allate, triamiphos, triapenthenol, triarathene, triarimol, triasulfuron, triazamate, triazbutil, triaziflam, triazophos, triazoxide, tribenuron, tribenuron-methyl, tribufos, tributyltin oxide, tricamba, trichlamide, trichlorfon, trichlormetaphos-3, trichloronat, triclopyr, triclopyr-butotyl, triclopyr-ethyl, triclopyricarb, triclopyr-triethylammonium, tricyclazole, tridemorph, tridiphane, trietazine, trifenmorph, trifenofos, trifloxystrobin, trifloxysulfuron, trifloxysulfuron-sodium, trifludimoxazin, triflumezopyrim, triflumizole, triflumuron, trifluralin, triflusulfuron, triflusulfuron-methyl, trifop, trifop-methyl, trifopsime, triforine, trihydroxytriazine, trimedlure, trimethacarb, trimeturon, trinexapac, trinexapac-ethyl, triprene, tripropindan, triptolide, tritac, triticonazole, tritosulfuron, trunc-call, tyclopyrazoflor, uniconazole, uniconazole-P, urbacide, uredepa, valerate, validamycin, valifenalate, valone, vamidothion, vangard, vaniliprole, vernolate, vinclozolin, warfarin, warfarin-potassium, warfarin-sodium, xiaochongliulin, xinjunan, xiwojunan, XMC, xylachlor, xylenols, xylylcarb, yishijing, zarilamid, zeatin, zengxiaoan, zeta-cypermethrin, zinc naphthenate, zinc phosphide, zinc thiazole, zineb, ziram, zolaprofos, zoxamide, zuomihuanglong, α-chlorohydrin, α-ecdysone, α-multistriatin, and α-naphthaleneacetic acid. For more information consult “THE PESTICIDE MANUAL” 15th Edition, edited by C D S Tomlin, copyright 2009 by British Crop Production Council, or its prior, or more recent editions.

In another embodiment, molecules of Formula One may also be used in combination (such as in a compositional mixture, or a simultaneous or sequential application) with one or more biopesticides. The term “biopesticide” is used for microbial biological pest control agents that are applied in a similar manner to chemical pesticides. Commonly these are bacterial, but there are also examples of fungal control agents, including Trichoderma spp. and Ampelomyces quisqualis (a control agent for grape powdery mildew). Bacillus subtilis are used to control plant pathogens. Weeds and rodents have also been controlled with microbial agents. One well-known insecticide example is Bacillus thuringiensis, a bacterial disease of Lepidoptera, Coleoptera, and Diptera. Because it has little effect on other organisms, it is considered more environmentally friendly than synthetic pesticides. Biological insecticides include products based on: entomopathogenic fungi (e.g. Metarhizium anisopliae); entomopathogenic nematodes (e.g. Steinernema feltiae); and entomopathogenic viruses (e.g. Cydia pomonella granulovirus).

Other examples of entomopathogenic organisms include, but are not limited to, baculoviruses, bacteria and other prokaryotic organisms, fungi, protozoa and Microsproridia. Biologically derived insecticides include, but not limited to, rotenone, veratridine, as well as microbial toxins; insect tolerant or resistant plant varieties; and organisms modified by recombinant DNA technology to either produce insecticides or to convey an insect resistant property to the genetically modified organism. In one embodiment, the molecules of Formula One may be used with one or more biopesticides in the area of seed treatments and soil amendments. The Manual of Biocontrol Agents gives a review of the available biological insecticide (and other biology-based control) products. Copping L. G. (ed.) (2004). The Manual of Biocontrol Agents (formerly the Biopesticide Manual) 3rd Edition. British Crop Production Council (BCPC), Farnham, Surrey UK.

In another embodiment, the above possible combinations may be used in a wide variety of weight ratios. For example, a two component mixture, the weight ratio of a molecule of Formula One to another compound, can be from about 100:1 to about 1:100; in another example the weight ratio can be about 50:1 to about 1:50; in another example the weight ratio can be about 20:1 to about 1:20; in another example the weight ratio can be about 10:1 to about 1:10; in another example the weight ratio can be about 5:1 to 1:5; in another example the weight ratio can be about 3:1 to about 1:3; and in a final example the weight ratio can be about 1:1. However, preferably, weight ratios less than about 10:1 to about 1:10 are preferred. It is also preferred sometimes to use a three or four component mixture comprising one or more molecules of Formula One and one or more other compounds from the above possible combinations.

### Formulations

A pesticide is rarely suitable for application in its pure form. It is usually necessary to add other substances so that the pesticide can be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph no 2, 5th Edition by CropLife International (2002).

Pesticides are applied most often as aqueous suspensions or emulsions prepared from concentrated formulations of such pesticides. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The concentration of the pesticide is usually from about 10% to about 90% by weight. The carrier is usually selected from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates of pesticides comprise a convenient concentration of a pesticide, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums may also be added, to increase the density and viscosity of the aqueous carrier. It is often most effective to grind and mix the pesticide at the same time by preparing the aqueous mixture and homogenizing it in an implement such as a sand mill, ball mill, or piston-type homogenizer.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. They can be applied as a seed dressing or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait they also consume the pesticide. Baits may take the form of granules, gels, flowable powders, liquids, or solids. They can be used in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings or in special chambers.

Pesticides can be microencapsulated by suspending the pesticide particles or droplets in plastic polymers of various types. By altering the chemistry of the polymer or by changing factors in the processing, microcapsules can be formed of various sizes, solubility, wall thicknesses, and degrees of penetrability. These factors govern the speed with which the active ingredient within is released, which in turn, affects the residual performance, speed of action, and odor of the product.

Oil solution concentrates are made by dissolving pesticide in a solvent that will hold the pesticide in solution. Oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer comprising: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007, having patent application Ser. No. 11/495,228. For ease of use, this embodiment will be referred to as “OIWE”.

For further information consult “Insect Pest Management” 2nd Edition by D. Dent, copyright CAB International (2000). Additionally, for more detailed information consult “Handbook of Pest Control—The Behavior, Life History, and Control of Household Pests” by Arnold Mallis, 9th Edition, copyright 2004 by GIE Media Inc.

### Other Formulation Components

Generally, when the molecules disclosed in Formula One are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alkyl ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules and water-dispersible granules.

Organic solvents are used mainly in the formulation of emulsifiable concentrates, oil-in-water emulsions, suspoemulsions, and ultra-low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of pesticides when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas.

Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenam; alginates; methyl cellulose; sodium carboxymethyl cellulose (SCMC); hydroxyethyl cellulose (HEC). Other types of anti-settling agents are based on modified starches, polyacrylates, polyvinyl alcohol and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g. plant and animal sources. Specific examples are: vegetable oils, seed oils, and esters thereof, also alkoxylated alkyl polyglucosides.

For further information, see “Chemistry and Technology of Agrochemical Formulations” edited by D. A. Knowles, copyright 1998 by Kluwer Academic Publishers. Also see “Insecticides in Agriculture and Environment—Retrospects and Prospects” by A. S. Perry, I. Yamamoto, I. Ishaaya, and R. Perry, copyright 1998 by Springer-Verlag.

### Pests

In general, the molecules of Formula One may be used to control pests e.g. ants, aphids, beetles, bristletails, cockroaches, crickets, earwigs, fleas, flies, grasshoppers, leafhoppers, lice, locusts, mites, moths, nematodes, scales, symphylans, termites, thrips, ticks, wasps, and whiteflies.

In another embodiment, the molecules of Formula One may be used to control pests in the Phyla Nematoda and/or Arthropoda.

In another embodiment, the molecules of Formula One may be used to control pests in the Subphyla Chelicerata, Myriapoda, and/or Hexapoda.

In another embodiment, the molecules of Formula One may be used to control pests in the Classes of Arachnida, Symphyla, and/or Insecta.

In another embodiment, the molecules of Formula One may be used to control pests of the Order Anoplura. A non-exhaustive list of particular genera includes, but is not limited to, Haematopinus spp., Hoplopleura spp., Linognathus spp., Pediculus spp., and Polyplax spp. A non-exhaustive list of particular species includes, but is not limited to, Haematopinus asini, Haematopinus suis, Linognathus setosus, Linognathus ovillus, Pediculus humanus capitis, Pediculus humanus humanus, and Pthirus pubis.

In another embodiment, the molecules of Formula One may be used to control pests in the Order Coleoptera. A non-exhaustive list of particular genera includes, but is not limited to, Acanthoscelides spp., Agriotes spp., Anthonomus spp., Apion spp., Apogonia spp., Aulacophora spp., Bruchus spp., Cerosterna spp., Cerotoma spp., Ceutorhynchus spp., Chaetocnema spp., Colaspis spp., Ctenicera spp., Curculio spp., Cyclocephala spp., Diabrotica spp., Hypera spp., Ips spp., Lyctus spp., Megascelis spp., Meligethes spp., Otiorhynchus spp., Pantomorus spp., Phyllophaga spp., Phyllotreta spp., Rhizotrogus spp., Rhynchites spp., Rhynchophorus spp., Scolytus spp., Sphenophorus spp., Sitophilus spp., and Tribolium spp. A non-exhaustive list of particular species includes, but is not limited to, Acanthoscelides obtectus, Agrilus planipennis, Anoplophora glabripennis, Anthonomus grandis, Ataenius spretulus, Atomaria linearis, Bothynoderes punctiventris, Bruchus pisorum, Callosobruchus maculatus, Carpophilus hemipterus, Cassida vittata, Cerotoma trifurcata, Ceutorhynchus assimilis, Ceutorhynchus napi, Conoderus scalaris, Conoderus stigmosus, Conotrachelus nenuphar, Cotinis nitida, Crioceris asparagi, Cryptolestes ferrugineus, Cryptolestes pusillus, Cryptolestes turcicus, Cylindrocopturus adspersus, Deporaus marginatus, Dermestes lardarius, Dermestes maculatus, Epilachna varivestis, Faustinus cubae, Hylobius pales, Hypera postica, Hypothenemus hampei, Lasioderma serricorne, Leptinotarsa decemlineata, Liogenys fuscus, Liogenys suturalis, Lissorhoptrus oryzophilus, Maecolaspis joliveti, Melanotus communis, Meligethes aeneus, Melolontha melolontha, Oberea brevis, Oberea linearis, Oryctes rhinoceros, Oryzaephilus mercator, Oryzaephilus surinamensis, Oulema melanopus, Oulema oryzae, Phyllophaga cuyabana, Popillia japonica, Prostephanus truncatus, Rhyzopertha dominica, Sitona lineatus, Sitophilus granarius, Sitophilus oryzae, Sitophilus zeamais, Stegobium paniceum, Tribolium castaneum, Tribolium confusum, Trogoderma variabile, and Zabrus tenebrioides.

In another embodiment, the molecules of Formula One may be used to control pests of the Order Dermaptera.

In another embodiment, the molecules of Formula One may be used to control pests of the Order Blattaria. A non-exhaustive list of particular species includes, but is not limited to, Blattella germanica, Blatta orientalis, Parcoblatta pennsylvanica, Periplaneta americana, Periplaneta australasiae, Periplaneta brunnea, Periplaneta fuliginosa, Pycnoscelus surinamensis, and Supella longipalpa.

In another embodiment, the molecules of Formula One may be used to control pests of the Order Diptera. A non-exhaustive list of particular genera includes, but is not limited to, Aedes spp., Agromyza spp., Anastrepha spp., Anopheles spp., Bactrocera spp., Ceratitis spp., Chrysops spp., Cochliomyia spp., Contarinia spp., Culex spp., Dasineura spp., Delia spp., Drosophila spp., Fannia spp., Hylemyia spp., Liriomyza spp., Musca spp., Phorbia spp., Tabanus spp., and Tipula spp. A non-exhaustive list of particular species includes, but is not limited to, Agromyza frontella, Anastrepha suspensa, Anastrepha ludens, Anastrepha obliqa, Bactrocera cucurbitae, Bactrocera dorsalis, Bactrocera invadens, Bactrocera zonata, Ceratitis capitata, Dasineura brassicae, Delia platura, Fannia canicularis, Fannia scalaris, Gasterophilus intestinalis, Gracillia perseae, Haematobia irritans, Hypoderma lineatum, Liriomyza brassicae, Melophagus ovinus, Musca autumnalis, Musca domestica, Oestrus ovis, Oscinella frit, Pegomya betae, Psila rosae, Rhagoletis cerasi, Rhagoletis pomonella, Rhagoletis mendax, Sitodiplosis mosellana, and Stomoxys calcitrans.

In another embodiment, the molecules of Formula One may be used to control pests of the Order Hemiptera. A non-exhaustive list of particular genera includes, but is not limited to, Adelges spp., Aulacaspis spp., Aphrophora spp., Aphis spp., Bemisia spp., Ceroplastes spp., Chionaspis spp., Chrysomphalus spp., Coccus spp., Empoasca spp., Lepidosaphes spp., Lagynotomus spp., Lygus spp., Macrosiphum spp., Nephotettix spp., Nezara spp., Philaenus spp., Phytocoris spp., Piezodorus spp., Planococcus spp., Pseudococcus spp., Rhopalosiphum spp., Saissetia spp., Therioaphis spp., Toumeyella spp., Toxoptera spp., Trialeurodes spp., Triatoma spp. and Unaspis spp. A non-exhaustive list of particular species includes, but is not limited to, Acrosternum hilare, Acyrthosiphon pisum, Aleyrodes proletella, Aleurodicus dispersus, Aleurothrixus floccosus, Amrasca biguttula biguttula, Aonidiella aurantii, Aphis gossypii, Aphis glycines, Aphis pomi, Aulacorthum solani, Bemisia argentifolii, Bemisia tabaci, Blissus leucopterus, Brachycorynella asparagi, Brevennia rehi, Brevicoryne brassicae, Calocoris norvegicus, Ceroplastes rubens, Cimex hemipterus, Cimex lectularius, Dagbertus fasciatus, Dichelops furcatus, Diuraphis noxia, Diaphorina citri, Dysaphis plantaginea, Dysdercus suturellus, Edessa meditabunda, Eriosoma lanigerum, Eurygaster maura, Euschistus heros, Euschistus servus, Helopeltis antonii, Helopeltis theivora, Icerya purchasi, Idioscopus nitidulus, Laodelphax striatellus, Leptocorisa oratorius, Leptocorisa varicornis, Lygus hesperus, Maconellicoccus hirsutus, Macrosiphum euphorbiae, Macrosiphum granarium, Macrosiphum rosae, Macrosteles quadrilineatus, Mahanarva frimbiolata, Metopolophium dirhodum, Mictis longicornis, Myzus persicae, Nephotettix cinctipes, Neurocolpus longirostris, Nezara viridula, Nilaparvata lugens, Parlatoria pergandii, Parlatoria ziziphi, Peregrinus maidis, Phylloxera vitifoliae, Physokermes piceae, Phytocoris californicus, Phytocoris relativus, Piezodorus guildinii, Poecilocapsus lineatus, Psallus vaccinicola, Pseudacysta perseae, Pseudococcus brevipes, Quadraspidiotus perniciosus, Rhopalosiphum maidis, Rhopalosiphum padi, Saissetia oleae, Scaptocoris castanea, Schizaphis graminum, Sitobion avenae, Sogatella furcifera, Trialeurodes vaporariorum, Trialeurodes abutiloneus, Unaspis yanonensis, and Zulia entrerriana.

In another embodiment, the molecules of Formula One may be used to control pests of the Order Hymenoptera. A non-exhaustive list of particular genera includes, but is not limited to, Acromyrmex spp., Atta spp., Camponotus spp., Diprion spp., Formica spp., Monomorium spp., Neodiprion spp., Pogonomyrmex spp., Polistes spp., Solenopsis spp., Vespula spp., and Xylocopa spp. A non-exhaustive list of particular species includes, but is not limited to, Athalia rosae, Atta texana, Iridomyrmex humilis, Monomorium minimum, Monomorium pharaonis, Solenopsis invicta, Solenopsis geminata, Solenopsis molesta, Solenopsis richtery, Solenopsis xyloni, and Tapinoma sessile.

In another embodiment, the molecules of Formula One may be used to control pests of the Order Isoptera. A non-exhaustive list of particular genera includes, but is not limited to, Coptotermes spp., Cornitermes spp., Cryptotermes spp., Heterotermes spp., Kalotermes spp., Incisitermes spp., Macrotermes spp., Marginitermes spp., Microcerotermes spp., Procornitermes spp., Reticulitermes spp., Schedorhinotermes spp., and Zootermopsis spp. A non-exhaustive list of particular species includes, but is not limited to, Coptotermes curvignathus, Coptotermes frenchi, Coptotermes formosanus, Heterotermes aureus, Microtermes obesi, Reticulitermes banyulensis, Reticulitermes grassei, Reticulitermes flavipes, Reticulitermes hageni, Reticulitermes hesperus, Reticulitermes santonensis, Reticulitermes speratus, Reticulitermes tibialis, and Reticulitermes virginicus.

In another embodiment, the molecules of Formula One may be used to control pests of the Order Lepidoptera. A non-exhaustive list of particular genera includes, but is not limited to, Adoxophyes spp., Agrotis spp., Argyrotaenia spp., Cacoecia spp., Caloptilia spp., Chilo spp., Chrysodeixis spp., Colias spp., Crambus spp., Diaphania spp., Diatraea spp., Earias spp., Ephestia spp., Epimecis spp., Feltia spp., Gortyna spp., Helicoverpa spp., Heliothis spp., Indarbela spp., Lithocolletis spp., Loxagrotis spp., Malacosoma spp., Peridroma spp., Phyllonorycter spp., Pseudaletia spp., Sesamia spp., Spodoptera spp., Synanthedon spp., and Yponomeuta spp. A non-exhaustive list of particular species includes, but is not limited to, Achaea janata, Adoxophyes orana, Agrotis ipsilon, Alabama argillacea, Amorbia cuneana, Amyelois transitella, Anacamptodes defectaria, Anarsia lineatella, Anomis sabulifera, Anticarsia gemmatalis, Archips argyrospila, Archips rosana, Argyrotaenia citrana, Autographa gamma, Bonagota cranaodes, Borbo cinnara, Bucculatrix thurberiella, Capua reticulana, Carposina niponensis, Chlumetia transversa, Choristoneura rosaceana, Cnaphalocrocis medinalis, Conopomorpha cramerella, Cossus cossus, Cydia caryana, Cydia funebrana, Cydia molesta, Cydia nigricana, Cydia pomonella, Darna diducta, Diatraea saccharalis, Diatraea grandiosella, Earias insulana, Earias vittella, Ecdytolopha aurantianum, Elasmopalpus lignosellus, Ephestia cautella, Ephestia elutella, Ephestia kuehniella, Epinotia aporema, Epiphyas postvittana, Erionota thrax, Eupoecilia ambiguella, Euxoa auxiliaris, Grapholita molesta, Hedylepta indicata, Helicoverpa armigera, Helicoverpa zea, Heliothis virescens, Hellula undalis, Keiferia lycopersicella, Leucinodes orbonalis, Leucoptera coffeella, Leucoptera malifoliella, Lobesia botrana, Loxagrotis albicosta, Lymantria dispar, Lyonetia clerkella, Mahasena corbetti, Mamestra brassicae, Maruca testulalis, Metisa plana, Mythimna unipuncta, Neoleucinodes elegantalis, Nymphula depunctalis, Operophtera brumata, Ostrinia nubilalis, Oxydia vesulia, Pandemis cerasana, Pandemis heparana, Papilio demodocus, Pectinophora gossypiella, Peridroma saucia, Perileucoptera coffeella, Phthorimaea operculella, Phyllocnistis citrella, Pieris rapae, Plathypena scabra, Plodia interpunctella, Plutella xylostella, Polychrosis viteana, Prays endocarpa, Prays oleae, Pseudaletia unipuncta, Pseudoplusia includens, Rachiplusia nu, Scirpophaga incertulas, Sesamia inferens, Sesamia nonagrioides, Setora nitens, Sitotroga cerealella, Sparganothis pilleriana, Spodoptera exigua, Spodoptera frugiperda, Spodoptera eridania, Thecla basilides, Tineola bisselliella, Trichoplusia ni, Tuta absoluta, Zeuzera coffeae, and Zeuzera pyrina.

In another embodiment, the molecules of Formula One may be used to control pests of the Order Mallophaga. A non-exhaustive list of particular genera includes, but is not limited to, Anaticola spp., Bovicola spp., Chelopistes spp., Goniodes spp., Menacanthus spp., and Trichodectes spp. A non-exhaustive list of particular species includes, but is not limited to, Bovicola bovis, Bovicola caprae, Bovicola ovis, Chelopistes meleagridis, Goniodes dissimilis, Goniodes gigas, Menacanthus stramineus, Menopon gallinae, and Trichodectes canis.

In another embodiment, the molecules of Formula One may be used to control pests of the Order Orthoptera. A non-exhaustive list of particular genera includes, but is not limited to, Melanoplus spp., and Pterophylla spp. A non-exhaustive list of particular species includes, but is not limited to, Anabrus simplex, Gryllotalpa africana, Gryllotalpa australis, Gryllotalpa brachyptera, Gryllotalpa hexadactyla, Locusta migratoria, Microcentrum retinerve, Schistocerca gregaria, and Scudderia furcata.

In another embodiment, the molecules of Formula One may be used to control pests of the Order Siphonaptera. A non-exhaustive list of particular species includes, but is not limited to, Ceratophyllus gallinae, Ceratophyllus niger, Ctenocephalides canis, Ctenocephalides felis, and Pulex irritans.

In another embodiment, the molecules of Formula One may be used to control pests of the Order Thysanoptera. A non-exhaustive list of particular genera includes, but is not limited to, Caliothrips spp., Frankliniella spp., Scirtothrips spp., and Thrips spp. A non-exhaustive list of particular sp. includes, but is not limited to, Frankliniella fusca, Frankliniella occidentalis, Frankliniella schultzei, Frankliniella williamsi, Heliothrips haemorrhoidalis, Rhipiphorothrips cruentatus, Scirtothrips citri, Scirtothrips dorsalis, and Taeniothrips rhopalantennalis, Thrips hawaiiensis, Thrips nigropilosus, Thrips orientalis, Thrips tabaci.

In another embodiment, the molecules of Formula One may be used to control pests of the Order Thysanura. A non-exhaustive list of particular genera includes, but is not limited to, Lepisma spp. and Thermobia spp.

In another embodiment, the molecules of Formula One may be used to control pests of the Order Acarina. A non-exhaustive list of particular genera includes, but is not limited to, Acarus spp., Aculops spp., Boophilus spp., Demodex spp., Dermacentor spp., Epitrimerus spp., Eriophyes spp., Ixodes spp., Oligonychus spp., Panonychus spp., Rhizoglyphus spp., and Tetranychus spp. A non-exhaustive list of particular species includes, but is not limited to, Acarapis woodi, Acarus siro, Aceria mangiferae, Aculops lycopersici, Aculus pelekassi, Aculus schlechtendali, Amblyomma americanum, Brevipalpus obovatus, Brevipalpus phoenicis, Dermacentor variabilis, Dermatophagoides pteronyssinus, Eotetranychus carpini, Notoedres cati, Oligonychus coffeae, Oligonychus ilicis, Panonychus citri, Panonychus ulmi, Phyllocoptruta oleivora, Polyphagotarsonemus latus, Rhipicephalus sanguineus, Sarcoptes scabiei, Tegolophus perseaflorae, Tetranychus urticae, and Varroa destructor.

In another embodiment, the molecules of Formula One may be used to control pest of the Order Symphyla. A non-exhaustive list of particular sp. includes, but is not limited to, Scutigerella immaculata.

In another embodiment, the molecules of Formula One may be used to control pests of the Phylum Nematoda. A non-exhaustive list of particular genera includes, but is not limited to, Aphelenchoides spp., Belonolaimus spp., Criconemella spp., Ditylenchus spp., Heterodera spp., Hirschmanniella spp., Hoplolaimus spp., Meloidogyne spp., Pratylenchus spp., and Radopholus spp. A non-exhaustive list of particular sp. includes, but is not limited to, Dirofilaria immitis, Heterodera zeae, Meloidogyne incognita, Meloidogyne javanica, Onchocerca volvulus, Radopholus similis, and Rotylenchulus reniformis.

Neonicotinoid-resistant insect is known in the art (see for example WO 2012/141754 A2). Compounds of the subject invention disclosed herein have the advantage of being superior or at least equal to insecticide activity against such neonicotinoid-resistant insect as compared to previously disclosed compounds. In some embodiments, the neonicotinoid-resistant insect has resistance to at least one of the insecticide selected from the group consisting of acetamiprid, clothianidin, dinotefuran, flupyradifurone (BYI 02960), imidacloprid, imidaclothiz, nitenpyram, thiacloprid, thiamethoxam, and combinations thereof. In other embodiments, the combination between the compounds of the subject invention and a second pesticide can be used for controlling such neonicotinoid-resistant insect. In further embodiments, the second pesticide is selected from the group consisting of acetamiprid, clothianidin, dinotefuran, flupyradifurone (BYI 02960), imidacloprid, imidaclothiz, nitenpyram, thiacloprid, and thiamethoxam.

### APPLICATIONS

Controlling pests of Phyla Nematoda, Arthropoda, and/or Mollusca generally means that pest populations, pest activity, or both, are reduced in an locus. This can come about when:

(a) pest populations are repulsed from a locus;

(b) pests are incapacitated in, or around, a locus; or

(c) pests are exterminated in, or around, a locus.

Of course, a combination of these results can occur. Generally, pest populations, activity, or both are desirably reduced more than fifty percent, preferably more than 90 percent, and most preferably more than 98 percent. Generally, the locus is not in, or on, a human; consequently, the locus is generally a non-human locus.

In another embodiment, the locus to which a molecule of Formula One is applied can be any locus that is inhabited, or that may become inhabited, or that may be traversed, by a pest of Phyla Nematoda, Arthropoda, and/or Mollusca. For example, the locus can be:

(a) where crops, trees, fruits, cereals, fodder species, vines, turf, and/or ornamental plants, are growing;

(b) where domesticated animals are residing;

(c) the interior or exterior surfaces of buildings (such as places where grains are stored);

(d) the materials of construction used in buildings (such as impregnated wood); and

(e) the soil around buildings.

Particular crop areas to use a molecule of Formula One include areas where apples, corn, sunflowers, cotton, soybeans, canola, wheat, rice, sorghum, barley, oats, potatoes, oranges, alfalfa, lettuce, strawberries, tomatoes, peppers, crucifers, pears, tobacco, almonds, sugar beets, beans and other valuable crops are growing or the seeds thereof are going to be planted. It is also advantageous to use ammonium sulfate with a molecule of Formula One when growing various plants.

In another embodiment, molecules of Formula One are generally used in amounts from about 0.0001 grams per hectare to about 5000 grams per hectare to provide control. In another embodiment, it is preferred that molecules of Formula One are used in amounts from about 0.001 grams per hectare to about 500 grams per hectare. In another embodiment, it is more preferred that molecules of Formula One are used in amounts from about 0.01 gram per hectare to about 50 grams per hectare.

The molecules of Formula One may be used in mixtures, applied simultaneously or sequentially, alone or with other compounds to enhance plant vigor (e.g. to grow a better root system, to better withstand stressful growing conditions). Such other compounds are, for example, compounds that modulate plant ethylene receptors, most notably 1-methylcyclopropene (also known as 1-MCP). Furthermore, such molecules may be used during times when pest activity is low, such as before the plants that are growing begin to produce valuable agricultural commodities. Such times include the early planting season when pest pressure is usually low.

The molecules of Formula One can be applied to the foliar and fruiting portions of plants to control pests. The molecules will either come in direct contact with the pest, or the pest will consume the pesticide when eating leaf, fruit mass, or extracting sap, that contains the pesticide. The molecules of Formula One can also be applied to the soil, and when applied in this manner, root and stem feeding pests can be controlled. The roots can absorb a molecule taking it up into the foliar portions of the plant to control above ground chewing and sap feeding pests.

Generally, with baits, the baits are placed in the ground where, for example, termites can come into contact with, and/or be attracted to, the bait. Baits can also be applied to a surface of a building, (horizontal, vertical, or slant surface) where, for example, ants, termites, cockroaches, and flies, can come into contact with, and/or be attracted to, the bait. Baits can comprise a molecule of Formula One.

The molecules of Formula One can be encapsulated inside, or placed on the surface of a capsule. The size of the capsules can range from nanometer size (about 100-900 nanometers in diameter) to micrometer size (about 10-900 microns in diameter).

Because of the unique ability of the eggs of some pests to resist certain pesticides, repeated applications of the molecules of Formula One may be desirable to control newly emerged larvae.

Systemic movement of pesticides in plants may be utilized to control pests on one portion of the plant by applying (for example by spraying an area) the molecules of Formula One to a different portion of the plant. For example, control of foliar-feeding insects can be achieved by drip irrigation or furrow application, by treating the soil with for example pre- or post-planting soil drench, or by treating the seeds of a plant before planting.

Seed treatment can be applied to all types of seeds, including those from which plants genetically modified to express specialized traits will germinate. Representative examples include those expressing proteins toxic to invertebrate pests, such as Bacillus thuringiensis or other insecticidal toxins, those expressing herbicide resistance, such as “Roundup Ready” seed, or those with “stacked” foreign genes expressing insecticidal toxins, herbicide resistance, nutrition-enhancement, drought resistance, or any other beneficial traits. Furthermore, such seed treatments with the molecules of Formula One may further enhance the ability of a plant to better withstand stressful growing conditions. This results in a healthier, more vigorous plant, which can lead to higher yields at harvest time. Generally, about 1 gram of the molecules of Formula One to about 500 grams per 100,000 seeds is expected to provide good benefits, amounts from about 10 grams to about 100 grams per 100,000 seeds is expected to provide better benefits, and amounts from about 25 grams to about 75 grams per 100,000 seeds is expected to provide even better benefits.

It should be readily apparent that the molecules of Formula One may be used on, in, or around plants genetically modified to express specialized traits, such as Bacillus thuringiensis or other insecticidal toxins, or those expressing herbicide resistance, or those with “stacked” foreign genes expressing insecticidal toxins, herbicide resistance, nutrition-enhancement, or any other beneficial traits.

The molecules of Formula One may be used for controlling endoparasites and ectoparasites in the veterinary medicine sector or in the field of non-human animal keeping. The molecules of Formula One are applied, such as by oral administration in the form of, for example, tablets, capsules, drinks, granules, by dermal application in the form of, for example, dipping, spraying, pouring on, spotting on, and dusting, and by parenteral administration in the form of, for example, an injection.

The molecules of Formula One may also be employed advantageously in livestock keeping, for example, cattle, sheep, pigs, chickens, and geese. They may also be employed advantageously in pets such as, horses, dogs, and cats. Particular pests to control would be fleas and ticks that are bothersome to such animals. Suitable formulations are administered orally to the animals with the drinking water or feed. The dosages and formulations that are suitable depend on the species.

The molecules of Formula One may also be used for controlling parasitic worms, especially of the intestine, in the animals listed above.

The molecules of Formula One may also be employed in therapeutic methods for human health care. Such methods include, but are limited to, oral administration in the form of, for example, tablets, capsules, drinks, granules, and by dermal application.

Pests around the world have been migrating to new environments (for such pest) and thereafter becoming a new invasive species in such new environment. The molecules of Formula One may also be used on such new invasive species to control them in such new environment.

The molecules of Formula One may also be used in an area where plants, such as crops, are growing (e.g. pre-planting, planting, pre-harvesting) and where there are low levels (even no actual presence) of pests that can commercially damage such plants. The use of such molecules in such area is to benefit the plants being grown in the area. Such benefits, may include, but are not limited to, improving the health of a plant, improving the yield of a plant (e.g. increased biomass and/or increased content of valuable ingredients), improving the vigor of a plant (e.g. improved plant growth and/or greener leaves), improving the quality of a plant (e.g. improved content or composition of certain ingredients), and improving the tolerance to abiotic and/or biotic stress of the plant.

Before a pesticide can be used or sold commercially, such pesticide undergoes lengthy evaluation processes by various governmental authorities (local, regional, state, national, and international). Voluminous data requirements are specified by regulatory authorities and must be addressed through data generation and submission by the product registrant or by a third party on the product registrant's behalf, often using a computer with a connection to the World Wide Web. These governmental authorities then review such data and if a determination of safety is concluded, provide the potential user or seller with product registration approval. Thereafter, in that locality where the product registration is granted and supported, such user or seller may use or sell such pesticide.

A molecule according to Formula One can be tested to determine its efficacy against pests. Furthermore, mode of action studies can be conducted to determine if said molecule has a different mode of action than other pesticides. Thereafter, such acquired data can be disseminated, such as by the internet, to third parties.

The headings in this document are for convenience only and must not be used to interpret any portion hereof.

We claim:

1. A compound having a structure of the following formula (“Formula One”) 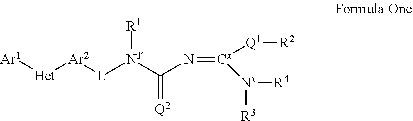
wherein:
(A) Ar¹ is selected from
(1) furanyl, phenyl, pyridazinyl, pyridyl, pyridinonyl, pyrimidinyl, thienyl, or
(2) substituted furanyl, substituted phenyl, substituted pyridazinyl, substituted pyridyl, substituted pyridinonyl, substituted pyrimidinyl, or substituted thienyl,
wherein said substituted furanyl, substituted phenyl, substituted pyridazinyl, substituted pyridyl, substituted pyridazinyl, substituted pyridinonyl, substituted pyrimidinyl, and substituted thienyl have one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)O(C₁-C₈ haloalkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)O(C₁-C₈ haloalkyl), (C₁-C₈ alkyl)S(C₁-C₈ alkyl), (C₁-C₈ alkyl)S(═O)(C₁-C₈ alkyl), C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)OC₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
(B) Het is a 5- or 6-membered, saturated or unsaturated, heterocyclic ring, containing one or more heteroatoms independently selected from nitrogen, sulfur, or oxygen, where said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O) C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, or S(═O)₂NR^{x}R^{y},
wherein each alkyl, haloalkyl, cycloalkyl, halocycloalkyl, alkoxy, haloalkoxy, alkenyl, cycloalkenyl, haloalkenyl, alkynyl, phenyl, and phenoxy substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)C(═O)NR^{x}R^{y}, (C₁-C₈ alkyl)NR^{x}R^{y}, C(═O)C(═O)(C₁-C₈ alkyl), C(═O)O(C₁-C₈ alkyl), C(═O)C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, or S(═O)₂NR^{x}R^{y};
(C) Ar² is selected from
(1) furanyl, phenyl, pyridazinyl, pyridyl, pyrimidinyl, thienyl, or
(2) substituted furanyl, substituted phenyl, substituted pyridazinyl, substituted pyridyl, substituted pyrimidinyl, or substituted thienyl,
wherein said substituted furanyl, substituted phenyl, substituted pyridazinyl, substituted pyridyl, substituted pyrimidinyl, and substituted thienyl, have one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)O(C₁-C₈ haloalkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) substituent may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)O(C₁-C₈ haloalkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
(D) R¹ is selected from H, CN, OH, SH, NO₂, C(═O)H, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1),
wherein each alkyl, haloalkyl, cycloalkyl, halocycloalkyl, alkoxy, haloalkoxy, alkenyl, cycloalkenyl, haloalkenyl, alkynyl, phenyl, phenoxy, and (Het-1) may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, and (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
(E) R² is selected from (J), H, C₁-C₈ alkyl, C₃-C₈ cycloalkyl, C₂-C₈ alkenyl, C₂-C₈ alkynyl, C(═O)(C₁-C₈ alkyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), C₁-C₈ alkyl)S(═O)₂(C₁-C₈ alkyl), (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈ alkyl)-C(═O)N(R^{x})(C₁-C₈ alkyl)-(Het-1), (C₁-C₈ alkyl)-C(═O)(Het-1), (C₁-C₈ alkyl)-C(═O)N(R^{x})(C₁-C₈ alkyl)N(R^{y})C(═O)OH, (C₁-C₈ alkyl)-C(═O)N(R^{x})(C₁-C₈ alkyl)N(R^{x})(R^{y}), (C₁-C₈ alkyl)-C(═O)N(R^{x})(C₁-C₈ alkyl)N(R^{y})C(═O)O—(C₁-C₈ alkyl), (C₁-C₈ alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(Het-1), (C₁-C₈ alkyl)S(═O)(Het-1), (C₁-C₈ alkyl)S(═O)₂(Het-1), or (C₁-C₈ alkyl)-O-(Het-1),
wherein each alkyl, cycloalkyl, phenyl, and (Het-1) are optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)OH, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
(F) R³ is selected from substituted C₃-C₈ cycloalkyl, substituted phenyl, substituted (C₁-C₈ alkyl)phenyl, substituted (C₁-C₈ alkyl)-O-phenyl, substituted (C₂-C₈ alkenyl)-O-phenyl, substituted (Het-1), substituted (C₁-C₈ alkyl)-(Het-1), or substituted (C₁-C₈ alkyl)-O-(Het-1),
wherein said substituted C₃-C₈ cycloalkyl, substituted phenyl, substituted (C₁-C₈ alkyl)phenyl, substituted (C₁-C₈ alkyl)-O-phenyl, substituted (C₂-C₈ alkenyl)-O-phenyl, substituted (Het-1), substituted (C₁-C₈ alkyl)-(Het-1), or substituted (C₁-C₈ alkyl)-O-(Het-1) has a substituent selected from NR^{x}C(═O)(C₁-C₈ alkyl), OH, SH, S(C₁-C₈ alkyl), S(C₃-C₈ cycloalkyl), S(C₁-C₈ haloalkyl), S(C₃-C₈ halocycloalkyl), C₁-C₈ haloalkyl, (C₁-C₈ alkyl)O(C₁-C₈ alkyl), (C₁-C₈ alkyl)O(C₁-C₈ alkyl), (C₃-C₈ cycloalkyl), unsubstituted (C₁-C₈ alkyl)O(C₁-C₈ alkyl)phenyl, (C₁-C₈ alkyl)O(C₁-C₈ alkyl)phenyl substituted with one or more of H, F, Cl, (C₁-C₈ haloalkyl), and (C₁-C₈ haloalkoxy), (C₁-C₈ alkyl)O(C₁-C₈ alkyl)benzothiazolyl, (C₁-C₈ alkyl)O(C₁-C₈ alkyl)benzoxazolyl, (C₁-C₈ alkyl)O(C₁-C₈ alkyl)thiophenyl, (C₁-C₈ alkyl)O(C₁-C₈ alkyl)thienopyrazolyl (C₁-C₈ alkyl)O(C₁-C₈ haloalkyl), (C₁-C₈ haloalkyl)O(C₁-C₈ alkyl), (C₁-C₈ haloalkyl)O(C₁-C₈ haloalkyl), (C₁-C₈ alkyl)S(C₁-C₈ alkyl), (C₁-C₈ alkyl)S(C₁-C₈ haloalkyl), (C₁-C₈ alkyl)S(═O)(C₁-C₈ alkyl), (C₁-C₈ alkyl)S(═O)(C₁-C₈ haloalkyl), (C₁-C₈ alkyl)S(═O)₂(C₁-C₈ alkyl), (C₁-C₈ alkyl)S(═O)₂(C₁-C₈ haloalkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), unsubstituted (C₁-C₈ alkyl)phenyl, (C₁-C₈ alkyl)pyridyl, (C₁-C₈ alkyl)phenyl substituted with one or more of F and (C₁-C₈)haloalkyl, or (C₁-C₈ alkyl)-O-phenyl; and
said substituted C₃-C₈ cycloalkyl, substituted phenyl, substituted (C₁-C₈ alkyl)phenyl, substituted (C₁-C₈ alkyl)-O-phenyl, substituted (C₂-C₈ alkenyl)-O-phenyl, substituted (Het-1), substituted (C₁-C₈ alkyl)-(Het-1), or substituted (C₁-C₈ alkyl)-O-(Het-1) may be optionally substituted with one or more substituents independently selected from H, F, Cl, Br, I, CN, OH, SH, SF₅, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ haloalkyl)O(C₁-C₈ alkyl), (C₁-C₈ haloalkyl)O(C₁-C₈ haloalkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, phenoxy, Si(C₁-C₈ alkyl)₃, SNR^{x}R^{y}, S(═O)NR^{x}R^{y}, S(═O)₂NR^{x}R^{y}, or (Het-1);
(G) R⁴ is selected from (J), H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-O—C(═O)NR^{x}R^{y}, (C₁-C₈ alkyl)-C(═O)N(R^{x})(C₁-C₈ alkyl)-(Het-1), (C₁-C₈ alkyl)-C(═O)(Het-1), (C₁-C₈ alkyl)-C(═O)N(R^{x})(C₁-C₈ alkyl)N(R^{y})C(═O)OH, (C₁-C₈ alkyl)-C(═O)N(R^{x})(C₁-C₈ alkyl)N(R^{x})(R^{y}), (C₁-C₈ alkyl)-C(═O)N(R^{x})(C₁-C₈ alkyl)N(R^{y})C(═O)O—(C₁-C₈ alkyl), (C₁-C₈ alkyl)-C(═O)N(R^{x})(C₁-C₈ alkyl)(N(R^{y})C(═O)O—(C₁-C₈ alkyl)C(═O)OH, (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-OC(═O)—(C₁-C₈ alkyl)N(R^{x})C(═O)O—(C₁-C₈ alkyl), (C₁-C₈ alkyl)-NR^{x}R^{y}, (C₁-C₈ alkyl)-S-(Het-1), (C₁-C₈ alkyl)S(═O)(Het-1), (C₁-C₈ alkyl)S(═O)₂(Het-1), or (C₁-C₈ alkyl)-O-(Het-1),
wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1);
(H) each of Q¹ and Q² is independently selected from O or S;
(I) R^{x} and R^{y} are independently selected from H, OH, SH, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, (C₁-C₈ alkyl)phenyl, (C₁-C₈ alkyl)-O-phenyl, C(═O)(Het-1), (Het-1), (C₁-C₈ alkyl)-(Het-1), (C₁-C₈ alkyl)-C(═O)—(C₁-C₈ alkyl), (C₁-C₈ alkyl)-OC(═O)—(C₁-C₈ alkyl), (C₁-C₈ alkyl)-O—C(═O)O—(C₁-C₈ alkyl), (C₁-C₈ alkyl)-C(═O)(Het-1), (C₁-C₈ alkyl)-C(═O)(Het-1)C(═O)O—(C₁-C₈ alkyl), (C₁-C₈ alkyl)-OC(═O)O—(C₁-C₈ alkyl), (C₁-C₈ alkyl)-OC(═O)—(C₁-C₈ alkyl), (C₁-C₈ alkyl)-OC(═O)—(C₃-C₈ cycloalkyl), (C₁-C₈ alkyl)-OC(═O)-(Het-1), (C₁-C₈ alkyl)-S-(Het-1), (C₁-C₈ alkyl)S(═O)(Het-1), (C₁-C₈ alkyl)S(═O)₂(Het-1), or (C₁-C₈ alkyl)-O-(Het-1),
wherein each alkyl, haloalkyl, cycloalkyl, halocycloalkyl, cycloalkoxy, halocycloalkoxy, alkoxy, haloalkoxy, alkenyl, cycloalkenyl, haloalkenyl, alkynyl, phenyl, and (Het-1), are optionally substituted with one or more substituents independently selected from H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)OH, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, halophenyl, phenoxy, and (Het-1),
or R^{x} and R^{y} together can optionally form a 5- to 7-membered saturated or unsaturated cyclic group which may contain one or more heteroatoms selected from nitrogen, sulfur, and oxygen, and where said cyclic group may be substituted with H, F, Cl, Br, I, CN, OH, SH, NO₂, oxo, thioxo, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, and (Het-1);
(J) R² and R⁴ may be a 1- to 4-membered saturated or unsaturated, hydrocarbyl link, which may contain one or more heteroatoms selected from nitrogen, sulfur, and oxygen, and together with C^{x}(Q¹)(N^{x}) forms a 4- to 7-membered cyclic structure, wherein said hydrocarbyl link may optionally be substituted with one or more substituents independently selected from R⁵, R⁶, and R⁷, wherein each R⁵, R⁶, and R⁷ is selected from H, F, Cl, Br, I, CN, OH, SH, NO₂, oxo, thioxo, NR^{x}R^{y}, C₁-C₈ alkyl, C₁-C₈ alkyl substituted with at least one OH, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, substituted phenyl, phenoxy, or (Het-1);
(K) (Het-1) is a 5- or 6-membered, saturated or unsaturated, heterocyclic ring, containing one or more heteroatoms independently selected from nitrogen, sulfur or oxygen, wherein said heterocyclic ring may also be substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy,
wherein each alkyl, cycloalkyl, alkoxy, alkenyl, alkynyl, phenyl, and phenoxy may be optionally substituted with one or more substituents independently selected from H, F, Cl, Br, I, CN, OH, SH, NO₂, oxo, thioxo, NR^{x}R^{y}, C₁-C₈ alkyl, C₁-C₈ haloalkyl, C₃-C₈ cycloalkyl, C₃-C₈ halocycloalkyl, C₃-C₈ cycloalkoxy, C₃-C₈ halocycloalkoxy, C₁-C₈ alkoxy, C₁-C₈ haloalkoxy, C₂-C₈ alkenyl, C₃-C₈ cycloalkenyl, C₂-C₈ haloalkenyl, C₂-C₈ alkynyl, S(C₁-C₈ alkyl), S(C₃-C₈ cycloalkyl), S(C₁-C₈ haloalkyl), S(C₃-C₈ halocycloalkyl), S(═O)(C₁-C₈ alkyl), S(═O)(C₁-C₈ haloalkyl), OSO₂(C₁-C₈ alkyl), S(═O)₂(C₁-C₈ alkyl), S(═O)₂(C₁-C₈ haloalkyl), OSO₂(C₁-C₈ alkyl), OSO₂(C₁-C₈ haloalkyl), C(═O)H, C(═O)NR^{x}R^{y}, (C₁-C₈ alkyl)NR^{x}R^{y}, C(═O)(C₁-C₈ alkyl), C(═O)O(C₁-C₈ alkyl), C(═O)(C₁-C₈ haloalkyl), C(═O)O(C₁-C₈ haloalkyl), C(═O)(C₃-C₈ cycloalkyl), C(═O)O(C₃-C₈ cycloalkyl), C(═O)(C₂-C₈ alkenyl), C(═O)O(C₂-C₈ alkenyl), (C₁-C₈ alkyl)O(C₁-C₈ alkyl), (C₁-C₈ alkyl)S(C₁-C₈ alkyl), (C₁-C₈ alkyl)S(═O)(C₁-C₈ alkyl), (C₁-C₈ alkyl)S(═O)₂(C₁-C₈ alkyl), (C₁-C₈ alkyl)OC(═O)(C₁-C₈ alkyl), (C₁-C₈ alkyl)OC(═O)O(C₁-C₈ alkyl), C(═O)(C₁-C₈ alkyl)C(═O)O(C₁-C₈ alkyl), (C₁-C₈ alkyl)C(═O)O(C₁-C₈ alkyl), (C₁-C₈ alkyl)C(═O)(C₁-C₈ alkyl), (C₁-C₈ alkyl)phenyl, (C₁-C₈ alkyl)-O-phenyl, phenyl, and phenoxy; and
(L) L is a linker selected from
(1) a bond,
(2) a saturated or unsaturated, substituted or unsubstituted, linear (C₁-C₄)hydrocarbyl linker, or
(3) a saturated or unsaturated, substituted or unsubstituted, cyclic (C₃-C₈)hydrocarbyl group linker,
wherein each of said linkers connects Ar² to N^{Y} and
wherein said substituted linear (C₁-C₄)hydrocarbyl linker and substituted cyclic (C₃-C₈)hydrocarbyl linker has one or more substituents independently selected from R⁸, R⁹, R¹⁰, R¹¹, and R¹², wherein each R⁸, R⁹, R¹⁰, R¹¹, and R¹², is selected from H, F, Cl, Br, I, CN, OH, SH, NO₂, oxo, thioxo, NR^{x}R^{y}, C₁-C₈ alkyl, C₁-C₈ haloalkyl, C₁-C₈ alkoxy, C₁-C₈ haloalkoxy, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₃-C₈ cycloalkenyl, C₃-C₈ halocycloalkenyl, (C₁-C₈ alkyl)O(C₁-C₈ alkyl), (C₁-C₈ alkyl)O(C₁-C₈ haloalkyl), S(C₁-C₈ alkyl), S(C₃-C₈ cycloalkyl), S(C₁-C₈ haloalkyl), S(C₃-C₈ halocycloalkyl), phenyl, or phenoxy.

2. The compound of claim 1, wherein:
(A) Ar¹ is a substituted phenyl, a substituted pyridyl, a substituted pyridazinyl, a substituted pyridinonyl, or a substituted pyrimidinyl, wherein said substituted phenyl, substituted pyridyl, substituted pyridazinyl, substituted pyridinonyl, and substituted pyrimidinyl have one or more substituents independently selected from H, CN, SF₅, C₁-C₆ alkyl, C₁-C₆ alkoxy, S(C₁-C₆ haloalkyl), C₁-C₆ haloalkyl and C₁-C₆ haloalkoxy;
(B) Het is selected from one of the following Het-A through Het-M 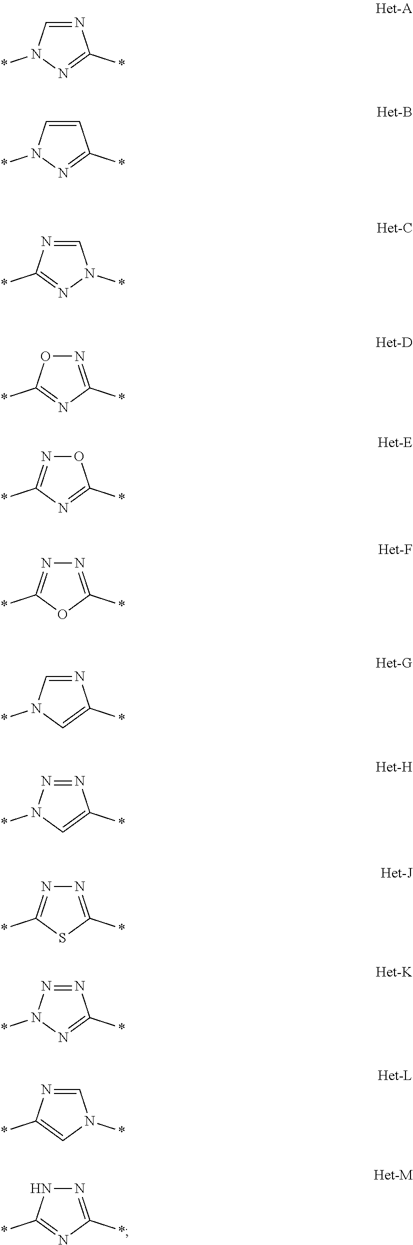
(C) Ar² is phenyl, pyridyl, pyrimidinyl, substituted phenyl, substituted pyridyl, or substituted pyrimidinyl, wherein said substituted phenyl, substituted pyridyl, and substituted pyrimidinyl have one or more substituents independently selected from H, F, Cl, Br, I, CN, NO₂, NR^{x}R^{y}, OH, C₁-C₆ alkoxy, (C₁-C₆ alkyl)O(C₁-C₆ alkyl), C₁-C₆ haloalkyl, and C₁-C₆ alkyl;
(D) R¹ is H, C₁-C₆ alkyl, C₃-C₆ cycloalkyl, or C₂-C₆ alkenyl, wherein said alkyl, cycloalkyl, or alkenyl is optionally substituted with a C₁-C₆ alkoxy, C₁-C₆ haloalkoxy, C₃-C₆ cycloalkyl, or C₃-C₆ halocycloalkyl;
(E) R² is (J), H, C₁-C₆ alkyl, or C₁-C₆ haloalkyl;
(F) R³ is a substituted phenyl, wherein said substituted phenyl has a substituent selected from N(CH₃)C(═O)CH₃, N(CH₂CH₃)C(═O)CH₃, OH, SH, S(C₁-C₆ alkyl), S(C₁-C₆ haloalkyl), C₁-C₆ haloalkyl, (C₁-C₆ alkyl)S(C₁-C₆ alkyl), (C₁-C₆ alkyl)S(C₁-C₆ haloalkyl), (C₁-C₆ alkyl)O(C₁-C₆ haloalkyl), (C₁-C₈ haloalkyl)O(C₁-C₈ alkyl), or (C₁-C₆ alkyl)O(C₁-C₆ alkyl); and
said substituted phenyl is optionally substituted with one or more substituents independently selected from H, F, Cl, Br, I, CN, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ cycloalkyl, C₁-C₆ halocycloalkyl, C₁-C₆ alkoxy, C₁-C₆ haloalkoxy, phenyl, C(═O)(C₁-C₆ alkyl), C(═O)O(C₁-C₆ alkyl), (C₁-C₈ alkyl)S(C₁-C₈ alkyl), or (C₁-C₈ alkyl)O(C₁-C₈ alkyl);
(G) R⁴ is selected from (J), H, C₁-C₆ alkyl, or C₁-C₆ haloalkyl;
(H) Q¹ is S and Q² is O;
(I) R^{x} and R^{y} are independently selected from H, C(═O)(C₁-C₆ alkyl), C(═O)O(C₁-C₆ alkyl), C₁-C₆ alkyl, C₁-C₆ haloalkyl, C₃-C₆ cycloalkyl, C₃-C₆ halocycloalkyl, C₂-C₆ alkenyl, C₃-C₆ cycloalkenyl, C₂-C₆ haloalkenyl, C₂-C₆ alkynyl, and phenyl;
(J) R² and R⁴ may be a 1- to 4-membered saturated or unsaturated, hydrocarbyl link, which may contain one or more heteroatoms selected from nitrogen, sulfur, and oxygen, and together with C^{x}(Q¹)(N^{x}) forms a cyclic structure, wherein said hydrocarbyl link may optionally be substituted with one or more substituents independently selected from R⁵, R⁶, and R⁷, wherein each R⁵, R⁶, and R⁷ is selected from H, F, Cl, Br, I, CN, NO₂, NR^{x}R^{y}, OH, SH, S(C₁-C₆ alkyl), S(C₁-C₆ haloalkyl), C₁-C₆ alkyl, C₁-C₆ alkyl substituted with OH, C₁-C₆ haloalkyl, C₁-C₆ alkoxy, C₁-C₆ haloalkoxy, C₃-C₆ cycloalkyl, C₃-C₆ halocycloalkyl, phenyl, and oxo; and
(L) L is a linker selected from
(1) a bond,
(2) a saturated or unsaturated, substituted or unsubstituted, linear (C₁-C₄)hydrocarbyl linker, or
(3) a saturated or unsaturated, substituted or unsubstituted, cyclic (C₃-C₈)hydrocarbyl group linker,
wherein each of said linkers connects Ar² to N^{Y} and
wherein said substituted linear (C₁-C₄)hydrocarbyl linker and substituted cyclic (C₃-C₈)hydrocarbyl linker has one or more substituents independently selected from R⁸, R⁹, R¹⁰, R¹¹, and R¹², wherein each R⁸, R⁹, R¹⁰, R¹¹, and R¹², is selected from H, F, Cl, Br, I, CN, oxo, thioxo, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ alkoxy, C₁-C₆ haloalkoxy, C₂-C₆ alkenyl, C₂-C₆ haloalkenyl, C₂-C₆ alkynyl, C₃-C₆ cycloalkyl, C₃-C₆ halocycloalkyl, C₃-C₆ cycloalkenyl, C₃-C₆ halocycloalkenyl, or phenyl.

3. The compound of claim 1, having a structure of Formula One-A or Formula Two:

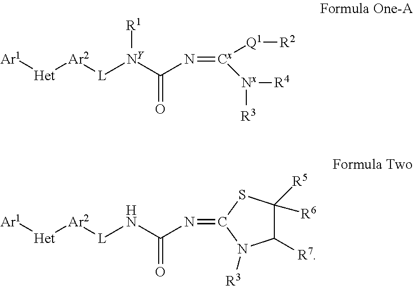

4. The compound of claim 1, having a structure selected from compounds listed in Table 1, Table 1A, Table 1B, and/or Table 1C.

5. The compound of claim 1 or 3, wherein
(A) Ar¹ is a substituted phenyl, a substituted pyridyl, or a substituted pyrimidinyl, wherein said substituted phenyl, substituted pyridyl, and substituted pyrimidinyl have one or more substituents independently selected from H, CN, C₁-C₆ alkyl, C₁-C₆ alkoxy, S(C₁-C₆ haloalkyl), C₁-C₆ haloalkyl and C₁-C₆ haloalkoxy;
(B) Het is triazolyl or pyrazolyl;
(C) Ar² is phenyl, pyridyl, pyrimidinyl, substituted phenyl, substituted pyridyl, or substituted pyrimidinyl, wherein said substituted phenyl, substituted pyridyl, and substituted pyrimidinyl have one or more substituents independently selected from H, F, Cl, Br, I, CN, NO₂, NR^{x}R^{y}, OH, C₁-C₆ alkoxy, (C₁-C₆ alkyl)O(C₁-C₆ alkyl), C₁-C₆ haloalkyl, and C₁-C₆ alkyl;
(D) R¹ is H, C₁-C₆ alkyl, C₃-C₆ cycloalkyl, or C₂-C₆ alkenyl, wherein said alkyl, cycloalkyl, or alkenyl is optionally substituted with a C₁-C₆ alkoxy, C₁-C₆ haloalkoxy, C₃-C₆ cycloalkyl, or C₃-C₆ halocycloalkyl;
(E) R² is (J), H, C₁-C₆ alkyl, or C₁-C₆ haloalkyl;
(F) R³ is a substituted phenyl, wherein said substituted phenyl has a substituent selected from N(CH₃)C(═O)CH₃, N(CH₂CH₃)C(═O)CH₃, OH, SH, S(C₁-C₆ alkyl), S(C₁-C₆ haloalkyl), C₁-C₆ haloalkyl, (C₁-C₆ alkyl)S(C₁-C₆ alkyl), (C₁-C₆ alkyl)S(C₁-C₆ haloalkyl), (C₁-C₆ alkyl)O(C₁-C₆ haloalkyl), (C₁-C₈ haloalkyl)O(C₁-C₈ alkyl), or (C₁-C₆ alkyl)O(C₁-C₆ alkyl); and
said substituted phenyl is optionally substituted with one or more substituents independently selected from H, F, Cl, Br, I, CN, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ cycloalkyl, C₁-C₆ halocycloalkyl, C₁-C₆ alkoxy, C₁-C₆ haloalkoxy, phenyl, C(═O)(C₁-C₆ alkyl), C(═O)O(C₁-C₆ alkyl), (C₁-C₈ alkyl)S(C₁-C₈ alkyl), or (C₁-C₈ alkyl)O(C₁-C₈ alkyl);
(G) R⁴ is selected from (J), H, C₁-C₆ alkyl, or C₁-C₆ haloalkyl;
(H) Q¹ is S and Q² is O;
(I) R^{x} and R^{y} are independently selected from H, C(═O)(C₁-C₆ alkyl), C₁-C₆ alkyl, C₁-C₆ haloalkyl, C₃-C₆ cycloalkyl, C₃-C₆ halocycloalkyl, C₂-C₆ alkenyl, C₃-C₆ cycloalkenyl, C₂-C₆ haloalkenyl, C₂-C₆ alkynyl, and phenyl;
(J) R² and R⁴ may be a 1- to 4-membered saturated or unsaturated, hydrocarbyl link, which may contain one or more heteroatoms selected from nitrogen, sulfur, and oxygen, and together with C^{x}(Q¹)(N^{x}) forms a cyclic structure, wherein said hydrocarbyl link may optionally be substituted with one or more substituents independently selected from R⁵, R⁶, and R⁷, wherein each R⁵, R⁶, and R⁷ is selected from H, F, Cl, Br, I, CN, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ alkoxy, C₁-C₆ haloalkoxy, C₃-C₆ cycloalkyl, C₃-C₆ halocycloalkyl, phenyl, and oxo; and
(L) L is a linker selected from
(1) a bond,
(2) a saturated or unsaturated, substituted or unsubstituted, linear (C₁-C₄)hydrocarbyl linker, or
(3) a saturated or unsaturated, substituted or unsubstituted, cyclic (C₃-C₈)hydrocarbyl group linker,
wherein each of said linkers connects Ar² to N^{Y} and
wherein said substituted linear (C₁-C₄)hydrocarbyl linker and substituted cyclic (C₃-C₈)hydrocarbyl linker has one or more substituents independently selected from R⁸, R⁹, R¹⁰, R¹¹, and R¹², wherein each R⁸, R⁹, R¹⁰, R¹¹, and R¹², is selected from H, F, Cl, Br, I, CN, oxo, thioxo, NO₂, NR^{x}R^{y}, OH, SH, S(C₁-C₆ alkyl), S(C₁-C₆ haloalkyl), C₁-C₆ alkyl, C₁-C₆ haloalkyl, C₁-C₆ alkoxy, C₁-C₆ haloalkoxy, C₂-C₆ alkenyl, C₂-C₆ haloalkenyl, C₂-C₆ alkynyl, C₃-C₆ cycloalkyl, C₃-C₆ halocycloalkyl, C₃-C₆ cycloalkenyl, C₃-C₆ halocycloalkenyl, or phenyl.

6. The compound of claim 3, having the structure of Formula Two and selected from compounds listed in Table 1 and the group consisting of H1, H2, H3, H4, H6, H7, H8, H10, H11, H12, H13, H14, H15, H16, H17, H20, and H21.

7. The compound of claim 1, having the structure of Formula Three, Formula Four, and/or Formula Five and selected from compounds listed in Table 1, 1A, 1B, and/or 1C and the group consisting of J30, J34, J39, J42, J49, J50, J60, J62, J66, J67, J72, J74, J81, J87, J88, J90, J94, J97, J117, J119, J120, J123, J124, J139, J141, J165, J167, J168, J173, J174, J178, J179, J180, J181, J182, J185, J186, J187, J188, J189, J190, J194, J195, J197, J199, J200, J203, J204, J205, J206, J214, J215, J220, J221, J222, J226, J228, J229, J230, J232, J233, J234, J236, J237, J240, J241, J244, J245, J249, J250, J256, J262, J265, J267, J271, J274, J276, J280, J281, J286, J289, J291, J297, J298, J299, J300, J303, J304, J306, J308, J309, J310, J311, J312, J314, J315, J16, J318, J319, J324, J325, J326, J327, J328, J344, J350, J362, J363, J364, J365, J366, J368, J510, J539, J570, J574, J645, J646, J647, J648, J649, J650, J652, J653, J654, J655, J656, J657, J659, J663, J684, J686, J716, J719, J742, J745, J747, J751, J778, J779, 780, J781, J782, J783, J784, J785, J786, J787, J788, J789, J790, J791, J792, J793, J794, J795, J796, J797, J798, and J799.

7. A compound selected from structures listed in Table 3.

9. A process comprising applying the compound of claim 1 to a locus to control a pest, in an amount sufficient to control such pest.

10. The process of claim 9, wherein said pest is beet armyworm (BAW), cabbage looper (CL), or yellow fever mosquito (YFM).

11. The compound of claim 1, comprising at least one ²H, at least one ¹⁴C, and/or at least one ¹⁹F.

12. A composition comprising the compound of claim 1 and at least one other compound having insecticidal, herbicidal, acaricidal, nematicidal, or fungicidal activity.

13. A composition comprising the compound of claim 1 and a seed.

14. A process comprising applying the compound of claim 1 to a genetically modified plant, or genetically-modified seed, which has been genetically modified to express one or more specialized traits.

15. A process comprising: orally administering; or topically applying; the compound of claim 1, to a non-human animal, to control endoparasites and/or ectoparasites.

16. A method for treating or protecting an non-human animal from infestation or infection by invertebrate pests which prises bringing the animal in contact with a pesticidally effective amount of at least one compound of Formula One according to claim 1, a stereoisomer thereof, or a veterinarily acceptable salt thereof.
